(19) United States
(12) Patent Application Publication
(10) Pub. No.: US 2026/0015358 A1
(43) Pub. Date: Jan. 15, 2026

(54) BICYCLIC DERIVATIVES, COMPOSITIONS AND USES THEREOF

(71) Applicant: Danatlas Pharmaceuticals Co., Ltd., Beijing (CN)

(72) Inventors: Wenlai ZHOU, Beijing (CN); Jincong ZHUO, Beijing (CN); Jingxi ZHANG, Beijing (CN)

(21) Appl. No.: 19/336,543

(22) Filed: Sep. 23, 2025

Related U.S. Application Data
(63) Continuation of application No. PCT/CN2024/083204, filed on Mar. 22, 2024.

(30) Foreign Application Priority Data
Mar. 23, 2023 (WO) PCT/CN2023/083493
May 16, 2023 (WO) PCT/CN2023/094609
Jul. 7, 2023 (WO) PCT/CN2023/106428
Jul. 27, 2023 (WO) PCT/CN2023/109596
Jan. 10, 2024 (WO) PCT/CN2024/071631
Feb. 7, 2024 (WO) PCT/CN2024/076713
Feb. 29, 2024 (WO) PCT/CN2024/079195

(51) Int. Cl. C07D 487/04; A61K 31/519; A61K 31/53; A61K 31/5377; A61K 31/553; A61P 35/00; C07B 59/00; C07D 519/00

(52) U.S. Cl. CPC C07D 487/04; A61K 31/519; A61K 31/53; A61K 31/5377; A61K 31/553; A61P 35/00; C07B 59/002; C07D 519/00

(57) ABSTRACT

The disclosure relates to tricyclic heterocyclic derivatives as shown in Formula (I), to pharmaceutical compositions comprising them, to a process for their preparation, and their use as therapeutic agents.

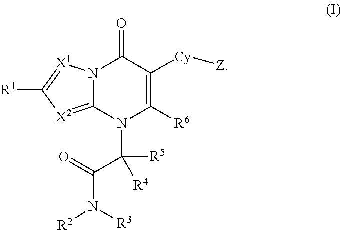

### CROSS REFERENCES TO RELATED APPLICATIONS

The present application is a continuation application of Internation Patent Application No. PCT/CN2024/083204, filed on Mar. 22, 2024, which claims the benefit of the priority of International Application No. PCT/CN2023/083493, filed Mar. 23, 2023, International Application No. PCT/CN2023/094609, filed May 16, 2023, International Application No. PCT/CN2023/106428, filed Jul. 7, 2023, International Application No. PCT/CN2023/109596, filed Jul. 27, 2023, International Application No. PCT/CN2024/071631, filed Jan. 10, 2024, International Application No. PCT/CN2024/076713, filed Feb. 7, 2024, International Application No. PCT/CN2024/079195, filed Feb. 29, 2024, each of which is hereby incorporated in its entirety.

### TECHNICAL FIELD

The present disclosure relates to bicyclic derivatives as WRN inhibitors. The present disclosure also relates to methods for preparing the bicyclic derivatives, pharmaceutical compositions, and their uses in the treatment of WRN-mediated diseases, e.g., cancers and other diseases.

### BACKGROUND

Numerous microsatellites are distributed prevalently in the genome of eukaryote. When DNA polymerase initiates replication at microsatellite sequences, base addition or deletion happens due to DNA polymerase slippage during replication. If DNA mismatch repair (MMR) is intact, the replication error is repaired and microsatellite-stable (MSS) is maintained. However, in the cell of mismatch repair deficiency (dMMR), these replication errors will be accumulated and finally lead to microsatellite instability-high (MSI-H) (Yuji Eso, 2019). dMMR/MSI-H is ubiquitous in many cancers (Bonneville R, 2017), such as colorectal, gastric, endometrial and adrenocortical cancers, etc.

Through large-scale CRISPR/Cas9 knockout in 517 cell lines and RNA interference (RNAi) silencing screens in 389 cell lines, Chan, E. M. et al. has identified the Werner Syndrome RecQ helicase (WRN) as being selectively required in vitro and in vivo, for the survival of cell lines with defective mismatch repair that has become MSI-H, yet dispensable in microsatellite stable (MSS) models (Chan, E. M., 2019). WRN is synthetic lethal with MSI-H cancers (Chan, E. M., 2019; Kategaya, L., 2019). Depletion of WRN leads to anti-proliferative effects and results in activation of multiple DNA damage signaling markers, induction of cell cycle arrest and apoptosis in MMR cancer models but not cancer cells with an intact MMR pathway.

Recently, the mechanism of WRN dependence in MSI-H cancers has been elucidated. WRN provides a DNA repair and maintenance function that is essential for MSI-H cell survival. In MSI-H cells, there is an increase in non-canonical secondary DNA structures creating a requirement for WRN for their resolution (van Wietmarschen N, 2020). In the absence of WRN (or upon WRN helicase inhibition) in MSI-H cancers, the replication machinery runs into the unresolved structures eventually leading to an increase in double-stranded breaks and cell death.

Therefore, inhibiting the WRN helicase is an attractive strategy for the treatment of dMMR/MSI-H cancers. There is a need to provide small molecule inhibitors for inhibiting the WRN helicase which is useful for treating cancer.

### SUMMARY

The present disclosure relates to, inter alia, compounds of Formula (I),

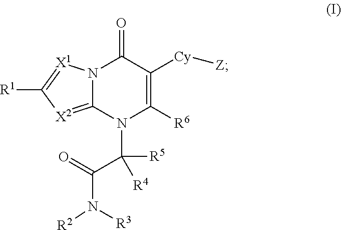

or a pharmaceutically acceptable salt, stereoisomer, solvate, tautomer, isotopic variant, prodrug, N-oxide or deuterated compound thereof; wherein the variables are as defined below.

In another aspect, provided herein is a pharmaceutical composition comprising a compound of formula (I), or pharmaceutically acceptable salt, stereoisomer, solvate, tautomer, isotopic variant, prodrug, N-oxide or deuterated compound thereof and at least one pharmaceutically acceptable carrier.

In another aspect, provided herein is a method of inhibiting WRN comprising:
contacting WRN with a compound of formula (I), or pharmaceutically acceptable salt, stereoisomer, solvate, tautomer, isotopic variant, prodrug, N-oxide or deuterated compound thereof.

In another aspect, provided herein is a method of treating cancers and other diseases comprising administering to a subject a therapeutically effective amount of a compound of formula (I), or pharmaceutically acceptable salt, stereoisomer, solvate, tautomer, isotopic variant, prodrug, N-oxide or deuterated compound thereof.

The details of one or more embodiments are set forth in the description below. Other features, objects, and advantages will be apparent from the description and from the claims.

### DETAILED DESCRIPTION

The present disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Before the present invention is further described, it is to be understood that the invention is not limited to the particular embodiments set forth herein, and it is also to be understood that the terminology used herein is for the purpose of describing particular embodiments only, and is not intended to be limiting.

The present disclosure provides, inter alia, a compound of formula (I) or a pharmaceutically acceptable salt, stereoisomer, solvate, tautomer, isotopic variant, prodrug, N-oxide or deuterated compound thereof, wherein:

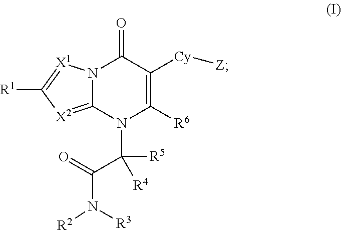

X¹ and X² are same or different, each is independently N or CR⁷;
Z is H, ring A, —C(O)R, —C(═NR¹⁰)R¹¹, —C(═NR¹⁰)NR¹¹BR^{11C}, —C(O)NR¹¹BR^{11C}, —C(O)OR^{11A}, —NR^{11C}C(O)R¹¹, —S(O)₂NR¹¹BR^{11C}, —NR^{11C}S(O)₂R¹¹, —S(O)₂NR^{11C}C(O)R¹¹, —C(O)NR^{11C}S(O)₂R¹¹, —C(O)NR^{11C}S(O)(═NR¹⁰)R¹¹, —S(O)R¹¹, —S(O)₂R¹¹, —S(O)(═NR¹⁰)R¹¹, —NR^{11C}C(O)NR¹¹BR^{11C}, —NR^{11C}C(O)OR^{11A}, or —C(O)C(O)NR¹¹BR^{11C};
ring A is C₃-C₁₀ cycloalkyl, 4-10 membered heterocycloalkyl, C₆-C₁₀ aryl or 5-10 membered heteroaryl; wherein, ring A is optionally substituted by 1, 2, 3, 4, 5 or 6 substituents R⁸;
R is C₁-C₈ alkyl, C₂-C₈ alkenyl, C₂-C₈ alkynyl, C₃-C₁₀ cycloalkyl, 4-10 membered heterocycloalkyl, C₆-C₁₀ aryl or 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl or 5-10 membered heteroaryl is optionally substituted by 1, 2, 3, 4, 5 or 6 substituents R⁹;
Cy is C₅-C₁₀ cycloalkylene, 5-14 membered heterocycloalkylene, C₆-C₁₀ arylene, or 5-10 membered heteroarylene; wherein, the Cy is optionally substituted by 1, 2, 3, 4 or 5 substituents independently selected from R¹²;
R¹ is halo, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, OR^{A}, SR^{A}, or NR^{C}R^{D}; wherein, the C₁-C₆ alkyl, C₂-C₆ alkenyl, C₂-C₆ alkynyl, C₃-C₁₀cycloalkyl, 4-10 membered heterocycloalkyl, C₆-C₁₀ aryl, or 5-10 membered heteroaryl is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b};
R² is C₁-C₆ alkyl, C₃-C₁₀ cycloalkyl, 4-10 membered heterocycloalkyl, C₆-C₁₀ aryl or 5-10 membered heteroaryl; wherein, the C₁-C₆ alkyl, C₃-C₁₀ cycloalkyl, 4-10 membered heterocycloalkyl, C₆-C₁₀ aryl or 5-10 membered heteroaryl is optionally substituted by 1, 2, 3, 4, or 5 substituents independently selected from R^{2A};
each R^{2A} is independently D, halo, CN, NO₂, N₃, SF₅, oxo, C₁-C₆ alkyl, C₂-C₆ alkenyl, C₂-C₆ alkynyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, OH, N₃, oxo, C₁-C₆ alkyl, C₁-C₆ haloalkyl, OC₁-C₆ alkyl, OC₁-C₆ haloalkyl, OC₁-C₆ alkyl-OH, C₃-C₅ cycloalkyl, or 4-5 membered heterocycloalkyl;
R³ is independently H, D, C(O)R^{B}, C₁-C₆ alkyl, C₁-C₆ haloalkyl, C₃-C₆ cycloalkyl or 4-6 membered heterocycloalkyl;
or R² and R³ together with the atom to which they are attached form 4-10 membered heterocycloalkyl or 5-10 membered heteroaryl; wherein, the 4-10 membered heterocycloalkyl or 5-10 membered heteroaryl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, OC₁-C₄ haloalkyl, C₃-C₅ cycloalkyl, or 4-5 membered heterocycloalkyl;
R⁴ and R⁵ are each independently H, D, halo, C₁-C₃ alkyl, C₁-C₃ haloalkyl, C₁-C₃ alkyl-OH, C₁-C₃ alkyl-CN, —C₁-C₃ alkyl-O—C₁-C₃ alkyl, or —C₁-C₃ alkyl-O—C₁-C₃ haloalkyl;
or R⁴ and R⁵ together with the atom to which they are attached form C₃-C₅ cycloalkyl or 4-5 membered heterocycloalkyl; wherein, the C₃-C₅ cycloalkyl or 4-5 membered heterocycloalkyl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl;
R⁶ is H, D, OR^{A}, NR^{C}R^{D}, SR^{A}, C(O)R^{B}, C₁-C₄ alkyl, C₂-C₄ alkenyl, C₂-C₄ alkynyl, C₃-C₅ cycloalkyl or 4-5 membered heterocycloalkyl; wherein, the C₁-C₄ alkyl, C₂-C₄ alkenyl, C₂-C₄ alkynyl, C₃-C₅ cycloalkyl or 4-5 membered heterocycloalkyl is optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl;
or R⁵ and R⁶ together with the atoms to which they are attached form a 5-7 membered heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, C₃-C₅cycloalkyl or 4-5 membered heterocycloalkyl;
R⁷ is independently H, D, halo, CN, OH, NH₂, C₁-C₃ alkyl, C₁-C₃ haloalkyl, OC₁-C₃ alkyl, OC₁-C₃ haloalkyl, C₃-C₅ cycloalkyl or 4-5 membered heterocycloalkyl;
each R⁸ is independently H, D, halo, CN, NO₂, N₃, oxo, C₁-C₆ alkyl, C₂-C₆ alkenyl, C₂-C₆ alkynyl, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl;

each R⁹ is independently H, D, halo, CN, NO₂, N₃, oxo, C₁-C₆ alkyl, C₂-C₆ alkenyl, C₂-C₆ alkynyl, C₁-C₄ alkyl-O—C₁-C₄ alkyl-Si(C₁-C₄ alkyl)₃, —NR^{C}R^{D}, OR^{A}, SR^{A}, C(O)R^{B}, C(O)OR^{A}. OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl;
each R¹⁰ is independently H, D, CN, OH, OMe, or C₁-C₄ alkyl optionally substituted by 1, 2, 3, 4 or 5 substituents independently selected from D, halo, CN, —OH, —O—C₁-C₄ alkyl, —OC₁-C₄ haloalkyl, NH₂, —NH(C₁-C₄ alkyl), or —N(C₁-C₄ alkyl)₂;
each R¹¹ is independently C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³;
or R¹⁰ and R¹¹ together with the atoms to which they are attached form 5-6 membered heteroaryl, 5-6 membered partially unsaturated heterocycloalkyl; wherein, the 5-6 membered heteroaryl or 5-6 membered partially unsaturated heterocycloalkyl is optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, or SC₁-C₄ alkyl;
each R^{11A} is independently H, D, C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³;
each R^{11B} is independently H, D, OR^{A}, C(O)R^{B}, C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³;
each R^{11C} is independently H, D, C₁-C₄ alkyl, C₂-C₄ alkenyl, C₂-C₄ alkynyl, C₃-C₇cycloalkyl, or 4-7 membered heterocycloalkyl; wherein, the C₁-C₄ alkyl, C₂-C₄ alkenyl, C₂-C₄ alkynyl, C₃-C₇ cycloalkyl or 4-7 membered heterocycloalkyl is optionally substituted by 1, 2, 3, 4, 5 or 6 substituents independently selected from D, halo, OH, oxo, CN, NO₂, N₃, C₁-C₄ alkyl, C₁-C₄ haloalkyl, OC₁-C₄ alkyl, or OC₁-C₄ haloalkyl;
or R^{11B} and R^{11C} together with the atom to which they are attached form a 4-10 membered heterocycloalkyl optionally substituted with 1, 2, or 3 substituents independently selected from D, halo, OH, NH₂, oxo, CN, C₁-C₄ alkyl, C₁-C₄ haloalkyl, OC₁-C₄ alkyl, OC₁-C₄ haloalkyl, NHC₁-C₄ alky, N(C₁-C₄ alkyl)₂, C₃-C₅ cycloalkyl or 4-5 membered heterocycloalkyl;
each R¹² is independently H, D, halo, CN, oxo, NO₂, C₁-C₆ alkyl, C₂-C₆ alkenyl, C₂-C₆ alkynyl, C₃-C₆ cycloalkyl, 4-6 membered heterocycloalkyl, NR^{C}R^{D}, OR^{A}, SR^{A}, C(O)R^{B}, S(O)R^{B}, S(O)₂R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, NR^{C}S(O)₂R^{B}, or NR^{C}S(O)₂NR^{C}R^{D}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)Rh;
wherein, two R¹² together with the same ring carbon atom to which they are attached form oxo, C₃-C₄ cycloalkyl, 4 membered heterocycloalkyl having 1 heteroatom selected from Si, N, O or S; wherein, the C₃-C₄ cycloalkyl, 4 membered heterocycloalkyl is optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl;
wherein, two R¹² together with the atoms to which they are attached form C₃-C₆ cycloalkyl, 4-6 membered heterocycloalkyl, phenylene or 5-6 membered heteroarylene; wherein, the C₃-C₆ cycloalkyl, 4-6 membered heterocycloalkyl, phenylene or 5-6 membered heteroarylene is optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl;
each R¹³ is independently H, D, halo, CN, NO₂, N₃, oxo, SF₅, C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl, C₀-C₆ alkyl-5-10 membered heteroaryl, C₁-C₄ alkyl-O—C₁-C₄ alkyl-Si(C₁-C₄ alkyl)₃, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}), NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, or OP(O)OR^{E}OR^{F}; wherein, the C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl, C₀-C₆ alkyl-5-10 membered heteroaryl is optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d};
Cy¹ is C₃-C₁₀ cycloalkyl, 4-10 membered heterocycloalkyl, C₆-C₁₀ aryl or 5-10 membered heteroaryl; wherein, the C₃-C₁₀ cycloalkyl, 4-10 membered heterocycloalkyl, C₆-C₁₀ aryl or 5-10 membered heteroaryl is optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d};
each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₄ alkenyl, C₂-C₄ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, C₁-C₄ alkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{C})NR^{c}R^{d}, NR^{c}C(═NR^{C})NR^{c}R^{d}, NR^{c}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or NR^{c}S(O)(═NR^{b})R^{b};
each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R^{B1};
each R^{B1} is independently D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f});
R^{C} and R^{D} are each independently H, D, OR^{a}, C(O)R^{b}, C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f});
or R^{C} and R^{D} together with the N atom to which they are attached form a 4-10 membered heterocycloalkyl optionally substituted with 1, 2, or 3 substituents independently selected from D, OH, oxo, CN, —NH₂, —NH(C₁-C₄ alkyl), —N(C₁-C₄ alkyl)₂, halo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, or OC₂-C₄ alkyl-O—C₁-C₄ haloalkyl;
R^{a} and R^{a1} are each independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy;
R^{b} and R^{b1} are each independently H, D, C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₆-C₁₀ aryl, C₃-C₁₀ cycloalkyl, 5-10 membered heteroaryl, or 4-10 membered heterocycloalkyl;
R^{c} and R^{d} are each independently H, D, C₁-C₄ alkyl, C₁-C₄ haloalkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₀-C₁₀ aryl-C₃-C₁₀ cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀ cycloalkyl, 4-10 membered heteroaryl-4-10 membered heterocycloalkyl, 4-10 membered heteroaryl-C₆-C₁₀ aryl, or bi(4-10 membered heteroaryl); wherein the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₀-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀ cycloalkyl, 4-10 membered heteroaryl-4-10 membered heterocycloalkyl, 4-10 membered heteroaryl-C₆-C₁₀ aryl, or bi(4-10 membered heteroaryl) is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—;
or R^{e} and R^{d} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
R^{E} and R^{e} are each independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl;
R^{F} and R^{f} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, or 4-10 membered heterocycloalkyl;
when

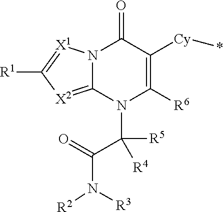

is

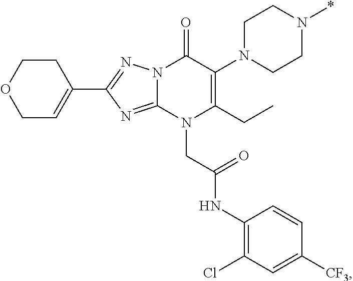 [SMILES: *N1CCN(c2c(CC)n(CC(=O)Nc3ccc(C(F)(F)F)cc3Cl)c3nc(C4=CCOCC4)nn3c2=O)CC1]

### and Z is not

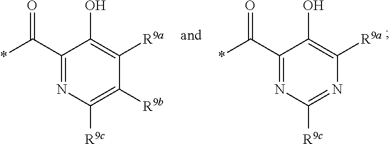

wherein, R^{9a} is H, F, Cl, or CH₃, OCF₃; R^{9b} is H, Cl, or CH₃; R^{9c} is H, or CH₃.

In some embodiments, X¹ is N and X² is N.

In some embodiments, X¹ is N and X² is CR⁷. In some embodiments, X¹ is N and X² is CH or CF.

In some embodiments, X¹ is CR⁷ and X² is N.

In some embodiments, X¹ is CR⁷ and X² is CR⁷.

In some embodiments, R⁷ is H, D, halo, CN, OH, NH₂, C₁-C₃ alkyl, C₁-C₃ haloalkyl, OC₁-C₃ alkyl, OC₁-C₃ haloalkyl, C₃-C₅ cycloalkyl or 4-5 membered heterocycloalkyl.

In some embodiments, R⁷ is H, D, halo, CN, C₁-C₃ alkyl, C₁-C₃ haloalkyl, OC₁-C₃ alkyl, OC₁-C₃ haloalkyl.

In some embodiments, R⁷ is H. In some embodiments, R⁷ is D.

In some embodiments, R⁷ is halo. In some embodiments, R⁷ is F, Cl, Br, or I. In some embodiments, R⁷ is F. In some embodiments, R⁷ is C₁.

In some embodiments, R⁷ is CN.

In some embodiments, R⁷ is C₁-C₃ alkyl. In some embodiments, R⁷ is CH₃, CH₂CH₃, CH₂CH₂CH₃, or CH(CH₃)₂. In some embodiments, R⁷ is CH₃.

In some embodiments, R⁷ is C₁-C₃ haloalkyl. In some embodiments, R⁷ is CH₂F, CHF₂, CF₃, CH₂CH₂F, CH₂CHF₂, CH₂CF₃, CF₂CH₃, CF₂CF₃, CF₂CH₂CH₃. In some embodiments, R⁷ is CH₂F, CHF₂, CF₃. In some embodiments, R⁷ is CH₂F. In some embodiments, R⁷ is CHF₂. In some embodiments, R⁷ is CF₃.

In some embodiments, R⁷ is OC₁-C₃ alkyl. In some embodiments, R⁷ is OCH₃. In some embodiments, R⁷ is OC₁-C₃ haloalkyl. In some embodiments, R⁷ is OCF₃.

In some embodiments, the moiety

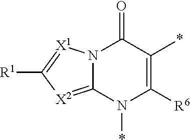

has the structure of

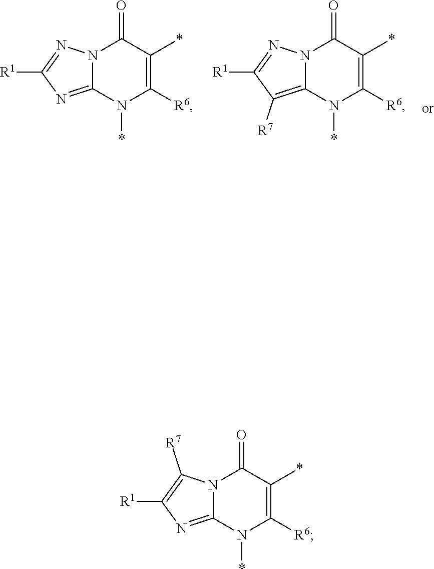

wherein R¹, R⁶, and R⁷ are as defined herein.

In some embodiments, the moiety

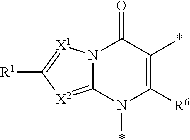

has the structure of

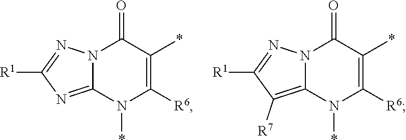

wherein R¹, R⁶, and R⁷ are as defined herein.

In some embodiments, the moiety

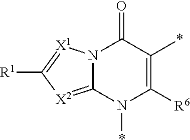

has the structure of

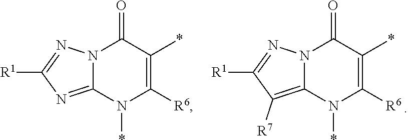

In some embodiments, the moiety

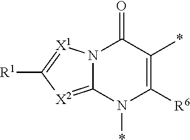

has the structure of

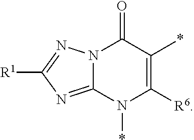

In some embodiments, the moiety

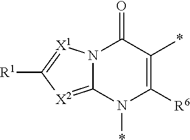

has the structure of

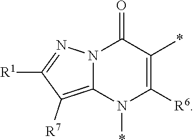

In some embodiments, the moiety

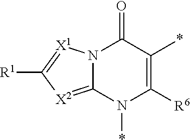

has the structure of

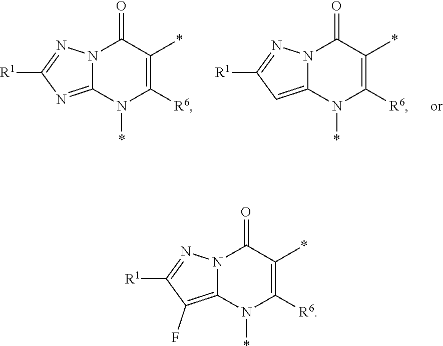

In some embodiments, R¹ is halo, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, OR^{A}, SR^{A}, or NR^{C}R^{D}; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is halo. In some embodiments, R¹ is F, Cl, Br, or I.

In some embodiments, R¹ is C₁-C₆ alkyl optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}. In some embodiments, R¹ is CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN, or

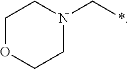

In some embodiments, R¹ is C₂-C₆ alkenyl optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is C₂-C₆ alkynyl optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is C₃-C₁₀ cycloalkyl optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is cyclopropyl, cyclobutyl, or cyclopentyl; each is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}. In some embodiments, R¹ is cyclopropyl, cyclobutyl, cyclopentyl.

In some embodiments, R¹ is 4-10 membered heterocycloalkyl (such as saturated 4-10 membered heterocycloalkyl or partially unsaturated 4-10 membered heterocycloalkyl) optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is azetidinyl, piperazinyl, morpholinyl, 3,6-dihydro-2H-pyranyl, 1,4-oxazepanyl, 2-oxa-6-azaspiro[3.3]heptanyl, 2-oxa-7-azaspiro[3.5]nonanyl, 2-oxa-8-azaspiro[4.5]decanyl, hexahydro-1H-furo[3,4-c]pyrrolyl, 2,3-dihydro-1H-pyrrolo[3,4-c]pyridinyl, 3,4-dihydro-2H-pyrano[2,3-b]pyridinyl, 2,3-dihydro-1H-pyrrolo[3,2-b]pyridinyl, 1,2,3,4,5,6-hexahydropyrrolo[3,4-c]pyrrolyl, 1,4,5,6-tetrahydropyrrolo[3,4-c]pyrazolyl, 5,6-dihydro-4H-pyrrolo[3,4-d]thiazolyl, 5,6,7,8-tetrahydroimidazo[1,2-a]pyrazinyl, hexahydro-3H-oxazolo[3,4-a]pyrazin-3-onyl, 5,6,7,8-tetrahydroimidazo[1,5-a]pyrazinyl, or 5,6,7,8-tetrahydro-[1,2,4]triazolo[4,3-a]pyrazinyl; each is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is

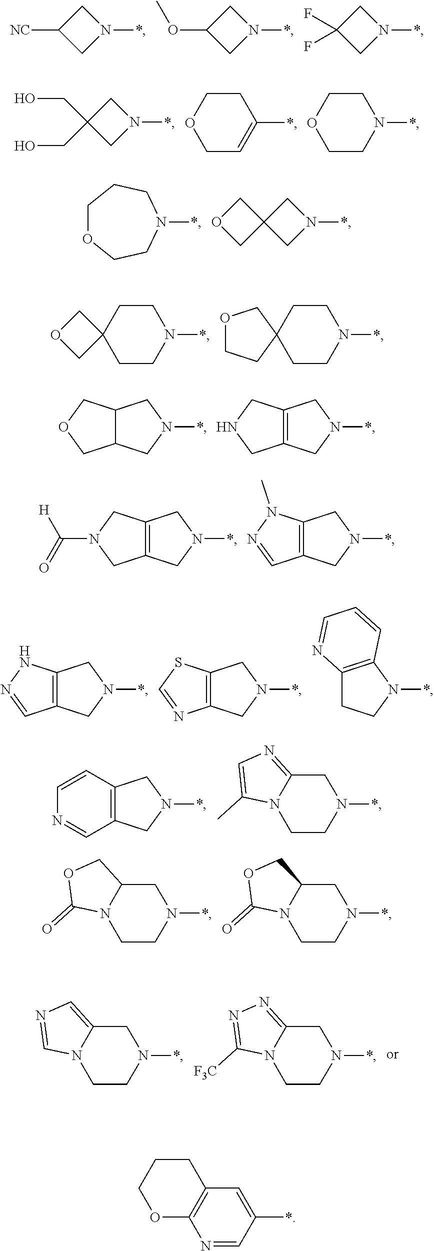

In some embodiments, R¹ is

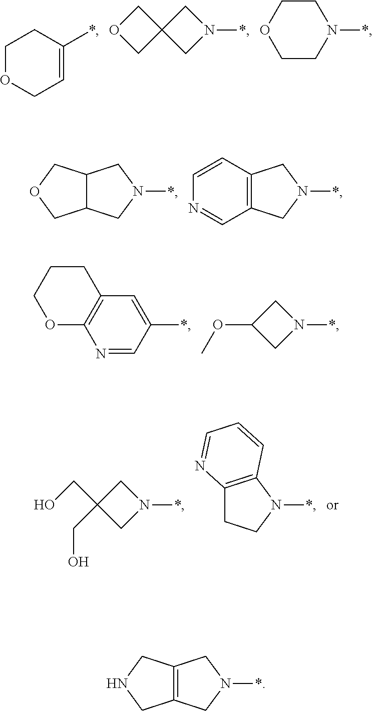

In some embodiments, R¹ is

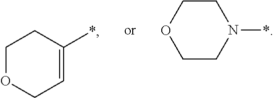

In some embodiments, R¹ is C₆-C₁₀ aryl (such as phenyl, naphthalenyl) optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is 5-10 membered heteroaryl optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

In some embodiments, R¹ is OR^{A}. In some embodiments, R¹ is OCH₃, OCH₂CH₃, OCH₂CH₂OCH₃, or OCH₂CH₂OCH₂CH₃.

In some embodiments, R¹ is SR^{A}. In some embodiments, R¹ is SCH₃, or SCH₂CH₃.

In some embodiments, R¹ is NR^{C}R^{D}.

In some embodiments, R² is C₁-C₆ alkyl, C₃-C₁₀ cycloalkyl, 4-10 membered heterocycloalkyl, C₆-C₁₀ aryl, or 5-10 membered heteroaryl; wherein, the C₁-C₆ alkyl, C₃-C₁₀ cycloalkyl, 4-10 membered heterocycloalkyl, C₆-C₁₀ aryl, or 5-10 membered heteroaryl is optionally substituted by 1, 2, 3, 4, or 5 substituents independently selected from R^{2A}.

In some embodiments, R² is C₁-C₆ alkyl optionally substituted by 1, 2, 3, 4, or 5 substituents independently selected from R^{2A}.

In some embodiments, R² is C₃-C₁₀ cycloalkyl optionally substituted by 1, 2, 3, 4, or 5 substituents independently selected from R^{2A}.

In some embodiments, R² is 4-10 membered heterocycloalkyl optionally substituted by 1, 2, 3, 4, or 5 substituents independently selected from R^{2A}.

In some embodiments, R² is C₆-C₁₀ aryl or 5-10 membered heteroaryl; each is optionally substituted by 1, 2, 3, 4, or 5 substituents independently selected from R^{2A}.

In some embodiments, R² is C₆-C₁₀ aryl optionally substituted by 1, 2, 3, 4, or 5 substituents independently selected from R^{2A}.

In some embodiments, R² is phenyl, naphthalenyl; each ring is optionally substituted by 1, 2, 3, 4, or 5 substituents independently selected from R^{2A}.

In some embodiments, R² is phenyl optionally substituted by 1, 2, or 3 substituents independently selected from R^{2A}.

In some embodiments, R² is

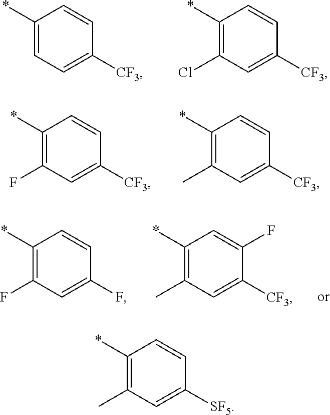

In some embodiments, R² is 5-10 membered heteroaryl optionally substituted by 1, 2, 3, 4, or 5 substituents independently selected from R^{2A}.

In some embodiments, R² is pyridinyl, thiophenyl, furanyl, 1H-imidazolyl, thiazolyl, 1H-pyrazolyl, isothiazolyl, isoxazolyl, or 1,3,4-thiadiazolyl; each is optionally substituted by 1, 2, 3, 4, or 5 substituents independently selected from R^{2A}.

In some embodiments, R² is

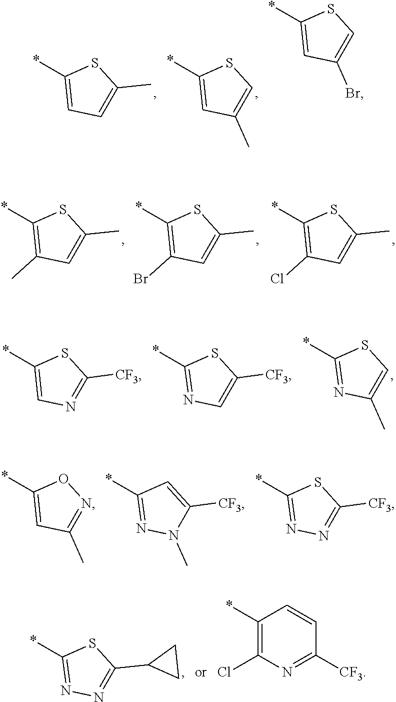

In some embodiments, each R^{2A} is independently D, halo, CN, NO₂, N₃, SF₅, oxo, C₁-C₆ alkyl, C₂-C₆ alkenyl, C₂-C₆ alkynyl, C₃-C₆ cycloalkyl, 4-6 membered heterocycloalkyl, OR^{a}, SR^{a}, C(O)R^{b}, C(O)OR^{a}, OC(O)R^{b}, C(O)NR^{c}R^{d}, NR^{c}C(O)R^{b}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, OH, N₃, oxo, OMe, OCF₃, OC₁-C₆ alkyl-OH, C₃-C₅ cycloalkyl, or 4-5 membered heterocycloalkyl.

In some embodiments, each R^{2A} is independently D, halo, CN, NO₂, N₃, SF₅, oxo, OR^{a}, C₁-C₆ alkyl, C₁-C₆ haloalkyl, C₃-C₆ cycloalkyl.

In some embodiments, each R^{2A} is independently D, halo, SF₅, C₁-C₆ alkyl, C₁-C₆ haloalkyl, C₃-C₆ cycloalkyl.

In some embodiments, each R^{2A} is independently D, F, Cl, Br, SF₅, —CH₃, —CF₃, cyclopropyl. In some embodiments, R³ is independently H, D, C(O)R^{B}, C₁-C₆ alkyl, C₁-C₆ haloalkyl, C₃-C₆ cycloalkyl or 4-6 membered heterocycloalkyl.

In some embodiments, R³ is independently H, D, C(O)R^{B}, C₁-C₆ alkyl, C₁-C₆ haloalkyl, C₃-C₆ cycloalkyl.

In some embodiments, R³ is H. In some embodiments, R³ is D.

In some embodiments, R³ is C(O)R^{B}, and R^{B} is C₁-C₆ alkyl. In some embodiments, R³ is C(O)CH₃, or C(O)CH₂CH₃. In some embodiments, R³ is C₁-C₆ alkyl. In some embodiments, R³ is CH₃, CH₂CH₃, CH₂CH₂CH₃, CH(CH₃)₂.

In some embodiments, R³ is C₁-C₆ haloalkyl. In some embodiments, R³ is CH₂F, CHF₂, CF₃, CH₂CH₂F, CH₂CHF₂, CH₂CF₃, CF₂CH₃, CF₂CF₃, CF₂CH₂CH₃.

In some embodiments, R³ is C₃-C₆ cycloalkyl. In some embodiments, R³ is cyclopropyl.

In some embodiments, R³ is 4-6 membered heterocycloalkyl.

In some embodiments, R² and R³ together with the atom to which they are attached form 4-10 membered heterocycloalkyl or 5-10 membered heteroaryl; wherein, the 4-10 membered heterocycloalkyl or 5-10 membered heteroaryl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl, C₃-C₅ cycloalkyl, or 4-5 membered heterocycloalkyl.

In some embodiments, R² and R³ together with the atom to which they are attached form 4-10 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl, C₃-C₅ cycloalkyl, or 4-5 membered heterocycloalkyl.

In some embodiments, R² and R³ together with the atom to which they are attached form 5-10 membered heteroaryl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl, C₃-C₅ cycloalkyl, or 4-5 membered heterocycloalkyl.

In some embodiments, R⁴ is H, D, halo, C₁-C₃ alkyl, C₁-C₃ haloalkyl, C₁-C₃ alkyl-OH, C₁-C₃ alkyl-CN, —C₁-C₃ alkyl-O—C₁-C₃ alkyl, or —C₁-C₃ alkyl-O—C₁-C₃ haloalkyl.

In some embodiments, R⁴ is H or D.

In some embodiments, R⁴ is H. In some embodiments, R⁴ is D. In some embodiments, R⁴ is halo (such as F, Cl, Br, I).

In some embodiments, R⁴ is C₁-C₃ alkyl. In some embodiments, R⁴ is CH₃, CH₂CH₃, CH₂CH₂CH₃, or CH(CH₃)₂. In some embodiments, R⁴ is CH₃.

In some embodiments, R⁴ is C₁-C₃ haloalkyl. In some embodiments, R⁴ is CH₂F, CHF₂, CF₃, CH₂CH₂F, CH₂CHF₂, CH₂CF₃, CF₂CH₃, CF₂CF₃, or CF₂CH₂CH₃. In some embodiments, R⁴ is CF₃. In some embodiments, R⁴ is C₁-C₃ alkyl-OH. In some embodiments, R⁴ is CH₂OH, CH₂CH₂OH, CH(OH)CH₃, CH₂CH₂CH₂OH, or CH(OH)CH₂CH₂OH.

In some embodiments, R⁴ is C₁-C₃ alkyl-CN. In some embodiments, R⁴ is CH₂CN, CH₂CH₂CN, or CH₂CH₂CH₂CN.

In some embodiments, R⁴ is —C₁-C₃ alkyl-O—C₁-C₃ alkyl. In some embodiments, R⁴ is CH₂OCH₃, CH₂CH₂OCH₃ or CH₂CH₂OCH₂CH₃.

In some embodiments, R⁴ is —C₁-C₃ alkyl-O—C₁-C₃ haloalkyl. In some embodiments, R⁴ is CH₂OCF₃, CH₂CH₂OCF₃ or CH₂CH₂OCH₂CF₃.

In some embodiments, R⁵ is H, D, C₁-C₃ alkyl, C₁-C₃ haloalkyl, C₁-C₃ alkyl-OH, C₁-C₃ alkyl-CN, —C₁-C₃ alkyl-O—C₁-C₃ alkyl, or —C₁-C₃ alkyl-O—C₁-C₃ haloalkyl.

In some embodiments, R⁵ is H or D.

In some embodiments, R⁵ is H. In some embodiments, R⁵ is D.

In some embodiments, R⁵ is C₁-C₃ alkyl. In some embodiments, R⁵ is CH₃, CH₂CH₃, CH₂CH₂CH₃, or CH(CH₃)₂. In some embodiments, R⁵ is CH₃.

In some embodiments, R⁵ is C₁-C₃ haloalkyl. In some embodiments, R⁵ is CH₂F, CHF₂, CF₃, CH₂CH₂F, CH₂CHF₂, CH₂CF₃, CF₂CH₃, CF₂CF₃, or CF₂CH₂CH₃. In some embodiments, R⁵ is CF₃. In some embodiments, R⁵ is C₁-C₃ alkyl-OH. In some embodiments, R⁵ is CH₂OH, CH₂CH₂OH, CH(OH)CH₃, CH₂CH₂CH₂OH, or CH(OH)CH₂CH₂OH.

In some embodiments, R⁵ is C₁-C₃ alkyl-CN. In some embodiments, R⁵ is CH₂CN, CH₂CH₂CN, or CH₂CH₂CH₂CN.

In some embodiments, R⁵ is —C₁-C₃ alkyl-O—C₁-C₃ alkyl. In some embodiments, R⁵ is CH₂CH₂OCH₃ or CH₂CH₂OCH₂CH₃.

In some embodiments, R⁵ is —C₁-C₃ alkyl-O—C₁-C₃ haloalkyl. In some embodiments, R⁵ is CH₂CH₂OCF₃ or CH₂CH₂OCH₂CF₃.

In some embodiments, R⁴ and R⁵ together with the atom to which they are attached form C₃-C₅ cycloalkyl or 4-5 membered heterocycloalkyl; wherein, the C₃-C₅ cycloalkyl or 4-5 membered heterocycloalkyl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl.

In some embodiments, R⁴ and R⁵ together with the atom to which they are attached form C₃-C₅ cycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl.

In some embodiments, R⁴ and R⁵ together with the atom to which they are attached form 4-5 membered heterocycloalkyl optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl.

In some embodiments, R⁶ is H, D, OR^{A}, NR^{C}R^{D}, SR^{A}, C(O)R^{B}, C₁-C₄ alkyl, C₂-C₄ alkenyl, C₂-C₄ alkynyl, C₃-C₅ cycloalkyl or 4-5 membered heterocycloalkyl; wherein, the C₁-C₄ alkyl, C₂-C₄ alkenyl, C₂-C₄ alkynyl, C₃-C₅ cycloalkyl or 4-5 membered heterocycloalkyl is optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl. In some embodiments, R⁶ is H, D, OR^{A}, or C₁-C₄ alkyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl.

In some embodiments, R⁶ is H. In some embodiments, R⁶ is D.

In some embodiments, R⁶ is OR^{A}. In some embodiments, R⁶ is OH, OCH₃, OCH₂CH₃.

In some embodiments, R⁶ is OH, the moiety

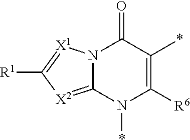

has tautomers selected from

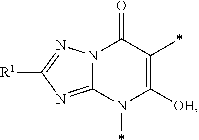

and/or

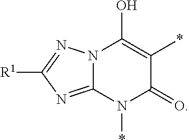

In some embodiments, R⁶ is NR^{C}R^{D}. In some embodiments, R⁶ is SR^{A}. In some embodiments, R⁶ is C(O)R^{B}.

In some embodiments, R⁶ is C₁-C₄ alkyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl. In some embodiments, R⁶ is CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN. In some embodiments, R⁶ is CH₃ or CH₂CH₃. In some embodiments, R⁶ is CH₂CH₃.

In some embodiments, R⁶ is C₂-C₄ alkenyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl.

In some embodiments, R⁶ is C₂-C₄ alkynyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl.

In some embodiments, R⁶ is C₃-C₅ cycloalkyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl.

In some embodiments, R⁶ is 4-5 membered heterocycloalkyl optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl, C₃-C₄ cycloalkyl or 4-membered heterocycloalkyl.

In some embodiments, R⁵ and R⁶ together with the atoms to which they are attached form a 5-7 membered heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, C₃-C₅ cycloalkyl or 4-5 membered heterocycloalkyl.

In some embodiments, Cy is C₅-C₁₀ cycloalkylene, 5-14 membered heterocycloalkylene, C₆-C₁₀ arylene, or 5-10 membered heteroarylene; wherein, the Cy is optionally substituted by 1, 2, 3, 4 or 5 substituents independently selected from R¹².

In some embodiments, Cy is C₅-C₁₀ cycloalkylene, 5-14 membered heterocycloalkylene; wherein, the Cy is optionally substituted by 1, 2, 3, 4 or 5 substituents independently selected from R¹².

In some embodiments, Cy is 5-14 membered heterocycloalkylene (such as saturated 5-14 membered heterocycloalkylene or partially unsaturated 5-14 membered heterocycloalkylene) optionally substituted by 1, 2, 3, 4 or 5 substituents independently selected from R¹². In some embodiments, Cy is 5-14 membered saturated heterocycloalkylene optionally substituted by 1, 2, 3, 4 or 5 substituents independently selected from R¹². In some embodiments, Cy is 5-14 membered partially unsaturated heterocycloalkylene optionally substituted by 1, 2, 3, 4 or 5 substituents independently selected from R¹².

In some embodiments, Cy is piperazinylene, piperidinylene, piperazin-2-onylene, azetidinylene, 2,6-diazaspiro[3.3]heptanylene, 2,7-diazaspiro[4.4]nonanylene, octahydropyrrolo[3,4-c]pyrrolylene, octahydro-2H-pyrazino[1,2-a]pyrazinylene, octahydro-4H-pyrazino[1,2-a]pyrazin-4-onylene; each is optionally substituted by 1, 2, 3, 4 or 5 substituents independently selected from R¹².

In some embodiments, Cy is

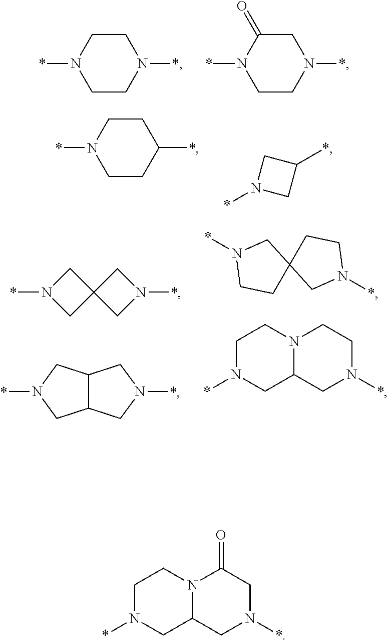

In some embodiments, Cy is

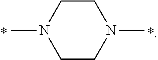

In some embodiments, Cy is C₆-C₁₀ arylene optionally substituted by 1, 2, 3, 4 or 5 substituents independently selected from R¹². In some embodiments, Cy is phenylene or naphthalenylene, each ring optionally substituted by 1, 2, 3, 4 or 5 substituents independently selected from R¹².

In some embodiments, Cy is 5-10 membered heteroarylene optionally substituted by 1, 2, 3, 4 or 5 substituents independently selected from R¹².

In some embodiments, each R¹² is independently H, D, halo, CN, oxo, NO₂, C₁-C₆ alkyl, C₂-C₆ alkenyl, C₂-C₆ alkynyl, C₃-C₆ cycloalkyl, 4-6 membered heterocycloalkyl, NR^{C}R^{D}, OR^{A}, SR^{A}, C(O)R^{B}, S(O)R^{B}, S(O)₂R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, NR^{C}S(O)₂R^{B}, or NR^{C}S(O)₂NR^{C}R^{D}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently H, D, halo, CN, oxo, C₁-C₆ alkyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently H, D, halo, CN, oxo, CH₃, CF₃, CH₂CH₃ or CH(CH₃)₂.

In some embodiments, each R¹² is independently H, D, halo, CN, oxo, NO₂. In some embodiments, each R¹² is independently H. In some embodiments, each R¹² is independently D. In some embodiments, each R¹² is independently halo (such as F, Cl, Br or I). In some embodiments, each R¹² is independently CN. In some embodiments, each R¹² is independently oxo. In some embodiments, each R¹² is independently NO₂.

In some embodiments, each R¹² is independently C₁-C₆ alkyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently C₂-C₆ alkenyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently C₂-C₆ alkynyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently C₃-C₆ cycloalkyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently 4-6 membered heterocycloalkyl optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

In some embodiments, each R¹² is independently NR^{C}R^{D}. In some embodiments, each R¹² is independently OR^{A}. In some embodiments, each R¹² is independently SR^{A}. In some embodiments, each R¹² is independently S(O)R^{B}. In some embodiments, each R¹² is independently S(O)₂R^{B}. In some embodiments, each R¹² is independently C(O)NR^{C}R^{D}. In some embodiments, each R¹² is independently NR^{C}C(O)R^{B}. In some embodiments, each R¹² is independently OC(O)NR^{C}R^{D}. In some embodiments, each R¹² is independently OC(O)OR^{A}. In some embodiments, each R¹² is independently NR^{C}C(O)NR^{C}R^{D}. In some embodiments, each R¹² is independently selected from NR^{c}C(O)OR^{A}. In some embodiments, each R¹² is independently NR^{C}S(O)₂R^{B}. In some embodiments, each R¹² is independently NR^{C}S(O)₂NR^{C}R^{D}.

In some embodiments, two R¹² together with the same ring carbon atom to which they are attached form oxo.

In some embodiments, two R¹² together with the same ring carbon atom to which they are attached form C₃-C₄ cycloalkyl optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, two R¹² together with the same ring carbon atom to which they are attached form 4 membered heterocycloalkyl having 1 heteroatom selected from Si, N, O or S optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, two R¹² together with the atoms to which they are attached form C₃-C₆ cycloalkyl optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, two R¹² together with the atoms to which they are attached form 4-6 membered heterocycloalkyl having 2 heteroatoms selected from Si, N, O or S optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, two adjacent R¹² together with the atoms to which they are attached form phenylene optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, two adjacent R¹² together with the atoms to which they are attached form 5-6 membered heteroarylene having 1, 2 or 3 heteroatoms selected from N, O or S optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl.

In some embodiments, Z is H, ring A, —C(O)R, —C(═NR¹⁰)R¹¹, —C(═NR¹⁰)NR^{11B}R^{11C}, —C(O)NR¹¹BR^{11C}, —C(O)OR^{11A}, —NR^{11C}C(O)R¹¹, —S(O)₂NR^{11B}R^{11C}, —NR¹ICS(O)₂R¹¹, S(O)₂NR^{11C}C(O)R¹¹, —C(O)NR^{11C}S(O)₂R¹¹, —C(O)NR^{11C}S(O)(═NR¹⁰)R¹¹, —S(O)R¹¹, —S(O)₂R¹¹, —S(O)(═NR¹⁰)R¹¹, —NR^{11C}C(O)NR^{11B}R^{11C}, —NR^{11C}C(O)OR^{11A}, or —C(O)C(O)NR¹¹BR^{11C}. In some embodiments, Z is —C(O)R, —C(═NR¹⁰)NR¹¹BR^{11C}, —C(O)NR¹¹BR^{11C}, —C(O)OR^{11A}, —NR^{11C}C(O)R¹¹, —S(O)₂NR¹¹BR^{11C}, —S(O)₂NR^{11C}C(O)R¹¹, —C(O)NR^{11C}S(O)₂R¹¹, —C(O)NR^{11C}S(O)(═NR¹⁰)R¹¹, —S(O)₂R¹¹, —S(O)(═NR¹⁰)R¹¹, or —C(O)C(O)NR^{11B}R^{11C}.

In some embodiments, Z is —C(O)R, —S(O)₂NR^{11C}C(O)R¹¹, —C(O)NR^{11C}S(O)₂R¹¹, —C(O)NR^{11C}S(O)(═NR¹⁰)R¹¹, or —C(O)C(O)NR¹¹BR^{11C}.

In some embodiments, Z is —C(O)R, or —C(O)C(O)NR^{11B}R^{11C}.

In some embodiments, Z is H.

In some embodiments, Z is ring A optionally substituted by 1, 2, 3, 4, 5 or 6 substituents R⁸.

In some embodiments, Z is —C(O)R. In some embodiments, Z is

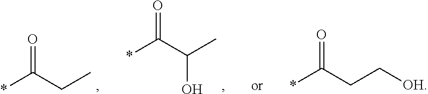

In some embodiments, Z is —C(═NR¹⁰)R¹¹. In some embodiments, Z is —C(═NR¹⁰)R¹¹, and R¹⁰ is OH, R¹¹ is C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂—C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³. In some embodiments, Z is —C(O)NR^{11C}S(O)₂R¹¹, and R¹⁰ is OH, R¹¹ is 5-10 membered heteroaryl optionally substituted by 1, 2, 3, 4, 5 or 6 substituents independently selected from R¹³. In some embodiments, Z is

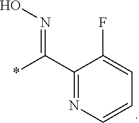 [SMILES: */C(=N\O)c1ncccc1F]

In some embodiments, Z is —C(═NR¹⁰)NR¹¹BR^{11C}. In some embodiments, Z is

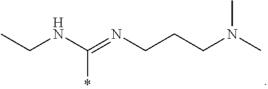 [SMILES: */C(=N\CCCN(C)C)NCC]

In some embodiments, Z is —C(O)NR¹¹BR^{11C}. In some embodiments, Z is —C(O)NHCH₂CH₃, —C(O)N(CH₃)₂,

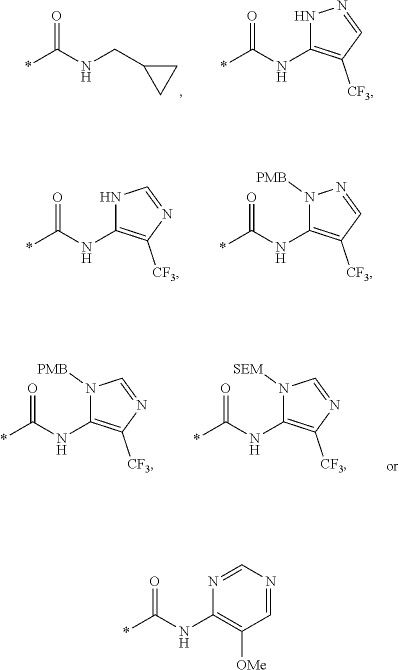

In some embodiments, Z is —C(O)OR^{11A}. In some embodiments, Z is

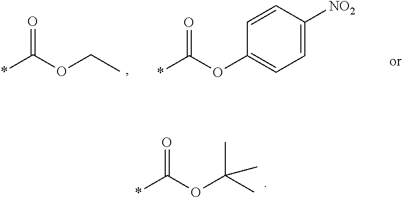

In some embodiments, Z is —NR^{11C}C(O)R¹¹. In some embodiments, Z is

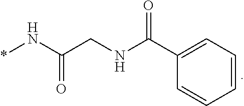 [SMILES: *NC(=O)CNC(=O)c1ccccc1]

In some embodiments, Z is —S(O)₂NR¹¹BR^{11C}. In some embodiments, Z is S(O)₂NH₂, or Z is S(O)₂NHEt.

In some embodiments, Z is —NR^{11C}S(O)₂R¹¹.

In some embodiments, Z is —S(O)₂NR^{11C}C(O)R¹¹. In some embodiments, Z is

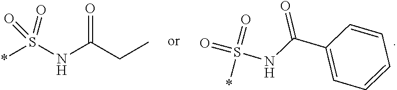

In some embodiments, Z is —C(O)NR^{11C}S(O)₂R¹¹. In some embodiments, Z is —C(O)NR^{11C}S(O)₂R¹¹, and R^{11C} is H, R¹¹ is C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, each is optionally substituted by 1, 2, 3, 4, 5 or 6 substituents independently selected from R¹³. In some embodiments, Z is —C(O)NR^{11C}S(O)₂R¹¹, and R^{11C} is H, R¹¹ is C₁-C₈ alkyl, C₃-C₁₀ cycloalkyl, 5-10 membered heteroaryl; wherein, each is optionally substituted by 1, 2, 3, 4, 5 or 6 substituents independently selected from R¹³. In some embodiments, Z is

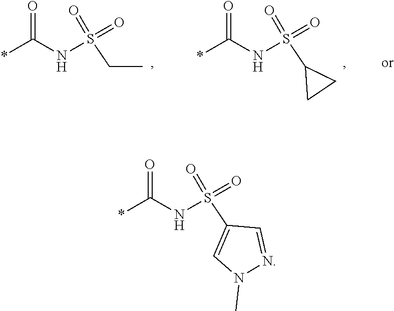

In some embodiments, Z is —C(O)NR^{11C}S(O)(═NR¹⁰)R¹¹.

In some embodiments, Z is —S(O)R¹¹.

In some embodiments, Z is —S(O)₂R¹¹. In some embodiments, Z is —S(O)₂CH₂CH₃.

In some embodiments, Z is —S(O)(═NR¹⁰)R¹¹.

In some embodiments, Z is —NR^{11C}C(O)NR^{11B}R^{11C}.

In some embodiments, Z is —NR^{11C}C(O)OR^{11A}.

In some embodiments, Z is —C(O)C(O)NR^{11B}R^{11C}.

In some embodiments, the compounds of Formula (I) are represented by compounds of Formula (IIa), (IIb), (IIc), (IId), (IIe), (IIf), (IIg), (IIh), (IIi), (IIj), or (IIk):

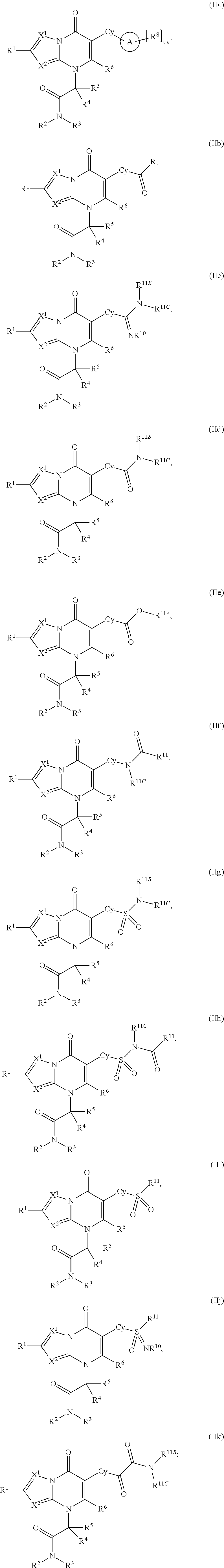

a pharmaceutically acceptable salt, stereoisomer, solvate, tautomer, isotopic variant, prodrug, N-oxide or deuterated compound thereof;
wherein, Cy, ring A, X¹, X², R, R¹, R², R³, R⁴, R⁵, R⁶, R⁸, R¹⁰, R¹¹, R^{11A}, R^{11B} and R^{11C} are defined with respect to Formula (I).

In some embodiments, ring A is C₃-C₁₀ cycloalkyl, 4-10 membered heterocycloalkyl, C₆-C₁₀ aryl or 5-10 membered heteroaryl; wherein, ring A is optionally substituted by 1, 2, 3, 4, 5 or 6 substituents R⁸.

In some embodiments, ring A is C₃-C₁₀ cycloalkyl. In some embodiments, ring A is 4-10 membered heterocycloalkyl. In some embodiments, ring A is C₆-C₁₀ aryl. In some embodiments, ring A is phenyl or naphthalenyl. In some embodiments, ring A is 5-10 membered heteroaryl.

In some embodiments, ring A is

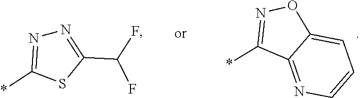 [SMILES: *c1nnc(C(F)F)s1 ; *c1noc2cccnc12]

In some embodiments, each R⁸ is independently H, D, halo, CN, NO₂, N₃, oxo, C₁-C₆ alkyl, C₂-C₆ alkenyl, C₂-C₆ alkynyl, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{c}C(O)NR^{C}R^{D}, NR^{c}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁸ is independently H, D, halo, CN, NO₂, N₃, oxo, C₁-C₆ alkyl, C₂-C₆ alkenyl, C₂-C₆ alkynyl, NR^{C}R^{D}, OR^{A}, SR^{A}, C(O)R^{B}, C(O)OR^{A}, C(O)NR^{C}R^{D}, NR^{C}C(O)R^{B}, NR^{C}C(O)NR^{C}R^{D}, NR^{C}C(O)OR^{A}, S(O)NR^{C}R^{D}, S(O)₂NR^{C}R^{D}, NR^{C}S(O)₂R^{B}, NR^{C}S(O)₂NR^{C}R^{D}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁸ is independently H, D, halo, CN, C₁-C₆ alkyl, C₂-C₆ alkenyl, C₂-C₆ alkynyl, NR^{C}R^{D}, OR^{A}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁸ is independently H, D, halo, CN, NR^{C}R^{D}, OR^{A}, or C₁-C₆ alkyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁸ is independently H, D, halo, CN. In some embodiments, each R⁸ is independently H. In some embodiments, each R⁸ is independently D. In some embodiments, each R⁸ is independently halo (such as F, Cl, Br or I). In some embodiments, each R⁸ is independently CN.

In some embodiments, each R⁸ is independently C₁-C₆ alkyl optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl. In some embodiments, each R⁸ is independently CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN. In some embodiments, R⁸ is CHF₂. In some embodiments, R⁸ is CH₃. In some embodiments, each R⁸ is independently NR^{C}R^{D}. In some embodiments, each R⁸ is independently NH₂, NHCH₃, N(CH₃)₂, NHCH₂CH₃, N(CH₂CH₃)₂, NHCH₂CH₂CH₃, N(CH₂CH₂CH₃)₂, NHCH(CH₃)₂, NHCH₂CH₂OH, N(CH₃)CH₂CH₂OH, NHCH₂CH₂OCH₃, N(CH₃)CH₂CH₂OCH₃.

In some embodiments, each R⁸ is independently OR^{A}. In some embodiments, each R⁸ is independently OH, OCH₃, OCH₂CH₃, OCH₂CH₂CH₃, OCH(CH₃)₂, OCH₂F, OCHF₂, OCF₃, OCH₂CH₂F, OCH₂CHF₂, OCH₂CF₃, OCF₂CF₃, OCH₂CH₂OH, or OCH₂CH₂OCH₃.

In some embodiments, the compounds of Formula (I) are represented by compounds of Formula (IIb):

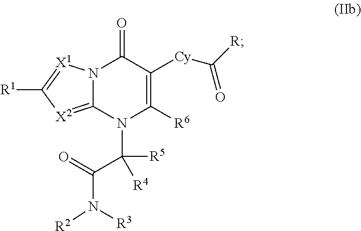

a pharmaceutically acceptable salt, stereoisomer, solvate, tautomer, isotopic variant, prodrug, N-oxide or deuterated compound thereof;
wherein, Cy, X¹, X², R, R¹, R², R³, R⁴, R⁵, and R⁶ are defined with respect to Formula (I). In some embodiments, R is C₁-C₈ alkyl, C₂-C₈ alkenyl, C₂-C₈ alkynyl, C₃-C₁₀ cycloalkyl, 4-10 membered heterocycloalkyl, C₆-C₁₀ aryl or 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents R⁹.

In some embodiments, R is C₁-C₈ alkyl, C₂-C₈ alkenyl, C₂-C₈ alkynyl, C₃-C₁₀ cycloalkyl, 4-10 membered heterocycloalkyl, or C₆-C₁₀ aryl; wherein, the C₁-C₈ alkyl, C₂-C₈ alkenyl, C₂-C₈ alkynyl, C₃-C₁₀ cycloalkyl, 4-10 membered heterocycloalkyl, or C₆-C₁₀ aryl is optionally substituted by 1, 2, 3, 4, 5 or 6 substituents R⁹.

In some embodiments, R is C₁-C₈ alkyl, C₂-C₈ alkenyl, or C₂-C₈ alkynyl; wherein, the C₁-C₈ alkyl, C₂-C₈ alkenyl, or C₂-C₈ alkynyl is optionally substituted by 1, 2, 3, 4, 5 or 6 substituents R⁹.

In some embodiments, R is C₁-C₈ alkyl optionally substituted by 1, 2, 3, 4, 5 or 6 substituents R⁹.

In some embodiments, R is C₂-C₈ alkenyl optionally substituted by 1, 2, 3, 4, 5 or 6 substituents R⁹. In some embodiments, R is

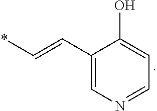 [SMILES: */C=C/c1cnccc1O]

In some embodiments, R is C₂-C₈ alkynyl optionally substituted by 1, 2, 3, 4, 5 or 6 substituents R⁹.

In some embodiments, R is C₃-C₁₀ cycloalkyl optionally substituted by 1, 2, 3, 4, 5 or 6 substituents R⁹.

In some embodiments, R is 4-10 membered heterocycloalkyl optionally substituted by 1, 2, 3, 4, 5 or 6 substituents R⁹.

In some embodiments, R is C₆-C₁₀ aryl optionally substituted by 1, 2, 3, 4, 5 or 6 substituents R⁹. In some embodiments, R is 5-10 membered heteroaryl optionally substituted by 1, 2, 3, 4, 5 or 6 substituents R⁹.

In some embodiments, the compounds of Formula (IIb) are represented by compounds of Formula (III):

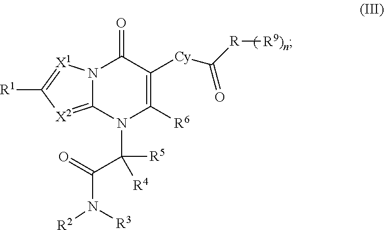

a pharmaceutically acceptable salt, stereoisomer, solvate, tautomer, isotopic variant, prodrug, N-oxide or deuterated compound thereof;
R is C₁-C₈ alkylene, C₂-C₈ alkenylene, C₂-C₈ alkynylene;
n is 1, 2, 3, 4, 5 or 6;
wherein, Cy, X¹, X², R¹, R², R³, R⁴, R⁵, R⁶ and R⁹ are defined with respect to Formula (I).

In some embodiments, the compounds of Formula (III) are represented by compounds of Formula (IIIa) or (IIIb):

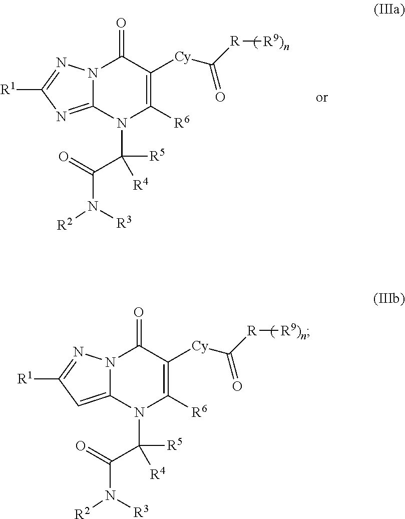

a pharmaceutically acceptable salt, stereoisomer, solvate, tautomer, isotopic variant, prodrug, N-oxide or deuterated compound thereof;
R is C₁-C₈ alkylene, C₂-C₈ alkenylene, or C₂-C₈ alkynylene;
n is 1, 2, 3, 4, 5 or 6;
wherein, Cy, R¹, R², R³, R⁴, R⁵, R⁶ and R⁹ are defined with respect to Formula (I).

In some embodiments, R is C₁-C₈ alkylene. In some embodiments, R is straight-chained or branched C₁-C₈ alkylene. In some embodiments, R is C₁-C₆ alkylene. In some embodiments, R is C₁-C₃ alkylene. In some embodiments, R is —CH₂—, —CH₂CH₂—, —CH(CH₃)—, —C(CH₃)₂—, —CF₂—. In some embodiments, R is —CH₂—.

In some embodiments, R is C₂-C₈ alkenylene. In some embodiments, R is C₂-C₆ alkenylene. In some embodiments, R is C₂-C₄ alkenylene. In some embodiments, ethenylene.

In some embodiments, R is C₂-C₈ alkynylene.

In some embodiments, each R⁹ is independently H, D, halo, CN, NO₂, N₃, oxo, —NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹.

In some embodiments, one of R⁹ is —NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; and the remaining R⁹ group is H, D, halo, or CN.

In some embodiments, one of R⁹ is —NR^{C}R^{D}, OR^{A}, NR^{C}C(O)R^{B}, NR^{C}C(O)NR^{C}R^{D}, NR^{C}C(O)OR^{A}, S(O)₂NR^{C}R^{D}, NR^{C}S(O)R^{B}, NR^{C}S(O)₂NR^{C}R^{D}, or Cy¹; and the remaining R⁹ group is H, D, halo, or CN.

In some embodiments, the compounds of Formula (III) are represented by compounds of Formula (IVa), (IVb), (IVc), (IVd), (IVe), (IVf) or (IVg):

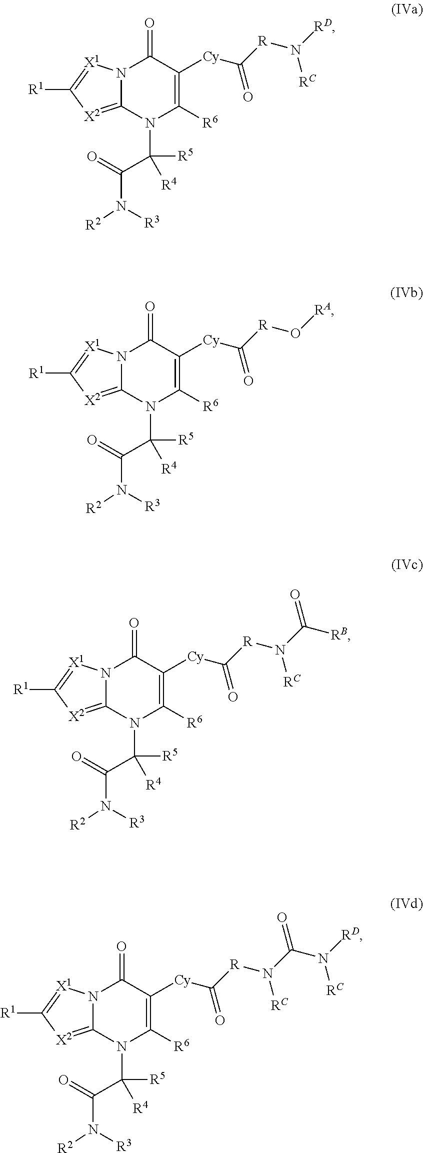

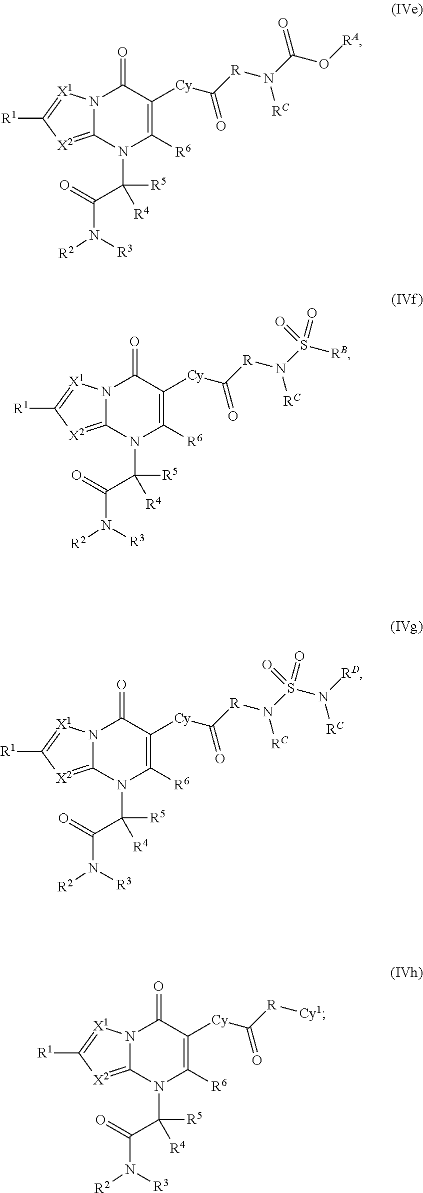

a pharmaceutically acceptable salt, stereoisomer, solvate, tautomer, isotopic variant, prodrug, N-oxide or deuterated compound thereof;
R is C₁-C₈ alkylene, C₂-C₈ alkenylene, or C₂-C₈ alkynylene; each is optionally substituted with 1, 2, 3, or 4 substituents independently selected from R⁹;
wherein, Cy, Cy¹, X¹, X², R¹, R², R³, R⁴, R⁵, R⁶, R⁹, R^{A}, R^{B}, R^{C} and R^{D} are defined with respect to Formula (I).

In some embodiments, R is straight-chained or branched C₁-C₃ alkylene optionally substituted with 1, 2, 3, or 4 R⁹, and each R⁹ is independently H, D, halo, CN.

In some embodiments, R is —CH₂—, —CD₂-, —CHF—, —CH₂CH₂—, —CH(CH₃)—, —C(CH₃)₂—, —CF₂—. In some embodiments, R is —CH₂—. In some embodiments, R is —CD₂-. In some embodiments, R is —CH(CH₃)—. In some embodiments, R is —CF₂—.

In some embodiments, R is C₂-C₃ alkenylene optionally substituted with 1, 2, 3, or 4 substituents independently selected from R⁹; and each R⁹ is independently H, D, halo, or CN. In some embodiments, R is C₂-C₃ alkenylene optionally substituted with 1, 2, 3, or 4 substituents independently selected from R⁹; and each R⁹ is independently H, D, halo, or CN. In some embodiments, R is ethenylene.

In some embodiments, R^{A} is H, D, C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, C₁-C₄ alkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{c})NR^{c}R^{d}, NR^{d}C(═NR^{c})NR^{c}R^{d}, NR^{d}C(═NR^{e})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or NR^{c}S(O)(═NR^{b})R^{b}.

In some embodiments, R^{A} is H, D, CH₃, CH₂CH₃, CH₂CH₂CH₃, C(CH₃)₃, cyclopropyl, cyclobutyl, azetidinyl, pyrrolidinyl, phenyl, pyridinyl, pyrimidinyl.

In some embodiments, R^{B} is C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R^{B1}.

In some embodiments, R^{B} is C₁-C₆ alkyl optionally substituted with 1, 2, 3, 4 or 5 substituents independently selected from R^{B1}.

In some embodiments, R^{B} is CH₃, CH₂CH₃, CH₂CH₂CH₃, CH(CH₃)₂, C(CH₃)₃, CH₂F, CHF₂, CF₃, CH₂OCH₃.

In some embodiments, R^{B} is C₃-C₁₀ cycloalkyl substituted with 1, 2, 3, 4 or 5 substituents independently selected from R^{B1}.

In some embodiments, R^{B} is cyclopropyl, cyclobutyl, cyclopentyl; each is optionally substituted with 1, 2, 3, 4 or 5 substituents independently selected from R^{B1}.

In some embodiments, R^{B} is 4-10 membered heterocycloalkyl substituted with 1, 2, 3, 4 or 5 substituents independently selected from R^{B1}.

In some embodiments, R^{B} is saturated 4-10 membered heterocycloalkyl or partially unsaturated 4-10 membered heterocycloalkyl substituted with 1, 2, 3, 4 or 5 substituents independently selected from R^{B1}.

In some embodiments, R^{B} is azetidinyl, oxetanyl, pyrrolidinyl, tetrahydrofuranyl, piperidinyl, morpholinyl, 2,3-dihydro-1H-pyrrolo[3,4-c]pyridinyl, indolinyl, 2H-pyran-2-onyl; each is optionally substituted with 1, 2, 3, 4 or 5 substituents independently selected from R^{B1}.

In some embodiments, R^{B} is

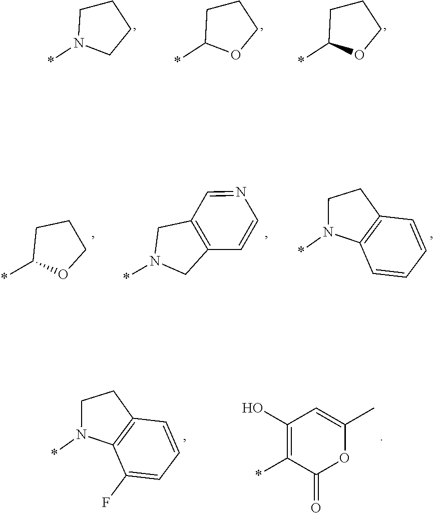

In some embodiments, R^{B} is C₆-C₁₀ aryl optionally substituted with 1, 2, 3, 4 or 5 substituents independently selected from R^{B1}.

In some embodiments, R^{B} is phenyl, naphthalenyl; each is optionally substituted with 1, 2, 3, 4 or 5 substituents independently selected from R^{B1}.

In some embodiments. R^{B} is phenyl optionally substituted with 1, 2, 3, 4 or 5 substituents independently selected from R^{B1}.

In some embodiments. R^{B} is

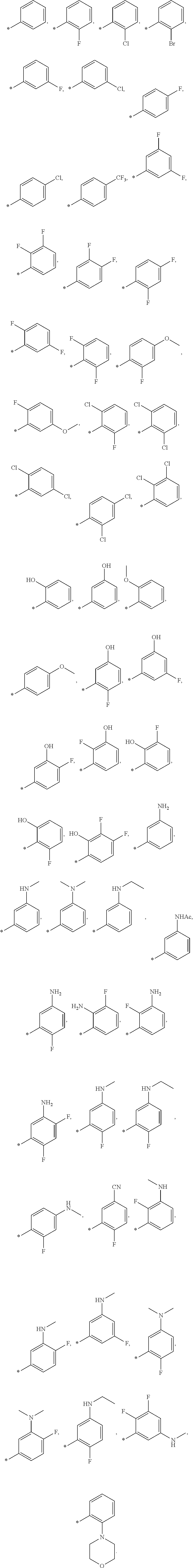

In some embodiments, R^{B} is 5-10 membered heteroaryl optionally substituted with 1, 2, 3, 4 or 5 substituents independently selected from R^{B1}.

In some embodiments, R^{B} is pyrrolyl, furanyl, thiophenyl, imidazolyl, oxazolyl, isoxazolyl, isothiazolyl, thiazolyl, tetrazolyl, pyrazolyl, 1,2,4-triazolyl, 1,2,3-triazolyl, thiadiazolyl, oxadiazolyl, pyridinyl, pyrimidinyl, pyrazinyl, pyridazinyl, indolyl, isoindolyl, indolizinyl, benzofuranyl, isobenzofuranyl, 1,2-dihydro-3H-pyrazol-3-onyl, 1,3-dihydro-2H-imidazol-2-onyl, oxazol-2 (3H)-onyl, benzo[b]thiophenyl, benzo[c]thiophenyl, indazolyl, benzo[d]imidazolyl, pyrrolo[1,2-b]pyridazinyl, pyrrolo[3,2-b]pyridinyl, pyrrolo[3,2-c]pyridinyl, pyrrolo[2,3-c]pyridinyl, pyrrolo[2,3-b]pyridinyl, pyrrolo[3,4-b]pyridinyl, pyrrolo[3,4-c]pyridinyl, benzo[d]isoxazolyl, benzo[d]oxazolyl, furo[3,2-b]pyridinyl, furo[3,2-c]pyridinyl, furo[2,3-c]pyridinyl, furo[2,3-b]pyridinyl, benzo[c]isoxazolyl, furo[3,4-b]pyridinyl, furo[3,4-c]pyridinyl, benzo[d]isothiazolyl, benzo[d]thiazolyl, thieno[3,2-b]pyridinyl, thieno[3,4-c]pyridinyl, benzo[d][1,2,3]triazolyl, pyrazolo[4,3-b]pyridinyl, pyrazolo[4,3-c]pyridinyl, pyrazolo[3,4-c]pyridinyl, pyrazolo[3,4-b]pyridinyl, imidazo[4,5-b]pyridinyl, imidazo[4,5-c]pyridinyl, imidazo[4,5-c]pyridinyl, imidazo[4,5-b]pyridinyl, pyrrolo[3,2-c]pyridazinyl, pyrrolo[3,2-d]pyrimidinyl, pyrrolo[2,3-b]pyrazinyl, pyrrolo[2,3-d]pyridazinyl, pyrrolo[2,3-d]pyrimidinyl, pyrrolo[2,3-c]pyridazinyl, pyrrolo[3,4-c]pyridazinyl, pyrrolo[3,4-d]pyrimidinyl, pyrrolo[3,4-b]pyrazinyl, pyrrolo[3,4-d]pyridazinyl, pyrrolo[3,4-d]pyrimidinyl, 6H-pyrrolo[3,4-c]pyridazinyl, thiazolo[5,4-b]pyridinyl, thiazolo[4,5-b]pyrazinyl, thiazolo[5,4-d]pyrimidinyl, thiazolo[4,5-d]pyrimidinyl, imidazo[2,1-b][1,3,4]thiadiazolyl, imidazo[5,1-b][1,3,4]thiadiazolyl, [1,2,4]triazolo[3,4-b][1,3,4]thiadiazolyl, [1,2,4]triazolo[5,1-b][1,3,4]thiadiazolyl, [1,2,4]triazolo[1,5-b][1,2,4]thiadiazolyl, imidazo[1,2-b][1,2,4]thiadiazolyl, thiazolo[5,4-d]thiazolyl, thiazolo[4,5-d]thiazolyl, thiazolo[3,2-b][1,2,4]triazolyl, isothiazolo[2,3-b][1,2,4]triazolyl, 2H-pyrazolo[4,3-d]thiazolyl, 4H-imidazo[4,5-d]thiazolyl, 2H-pyrazolo[3,4-d]thiazolyl, benzo[c][1,2,5]oxadiazolyl; each is optionally substituted with 1, 2, 3, 4 or 5 substituents independently selected from R^{B1}.

In some embodiments, R^{B} is pyrrolyl, furanyl, thiophenyl, imidazolyl, pyrazolyl, isoxazolyl, isothiazolyl, oxazolyl, thiazolyl, pyridinyl, pyrimidinyl, 1,2-dihydro-3H-pyrazol-3-onyl, pyrrolo[1,2-b]pyridazinyl, pyrrolo[2,3-b]pyridinyl, benzo[d]isoxazolyl, benzo[c][1,2,5]oxadiazolyl; each is optionally substituted with 1, 2, 3, 4 or 5 substituents independently selected from R^{B1}.

In some embodiments, R^{B} is

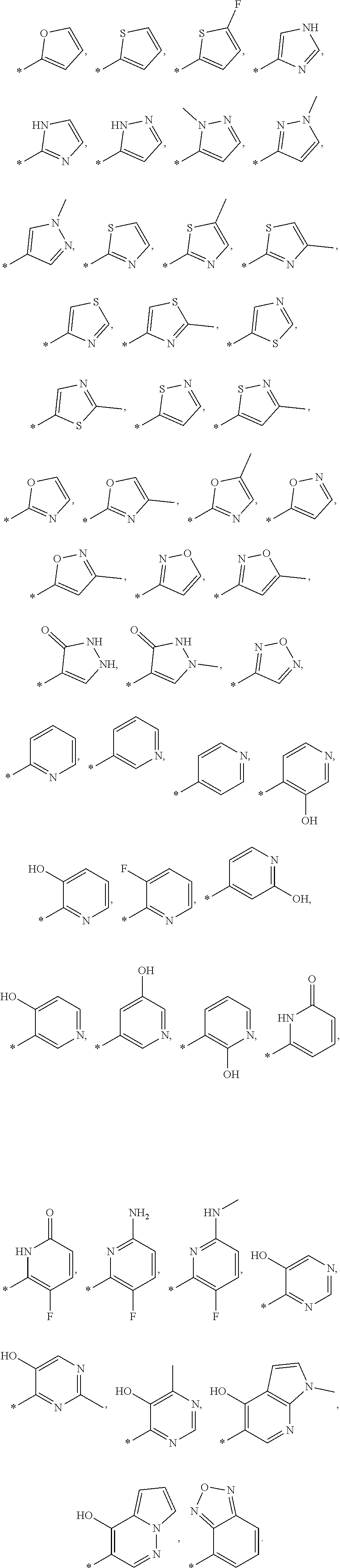

In some embodiments, R^{B} is C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; each is optionally substituted with 1, 2, 3, 4 or 5 substituents independently selected from R^{B1}.

In some embodiments, R^{B} is C₆-C₁₀ aryl-C₁-C₂ alkyl, 5-10 membered heteroaryl-C₁-C₂ alkyl, C₃-C₁₀ cycloalkyl-C₁-C₂ alkyl, or 4-10 membered heterocycloalkyl-C₁-C₂ alkyl; each is optionally substituted with 1, 2, 3, 4 or 5 substituents independently selected from R^{B1}.

In some embodiments, R^{B} is cyclopropylmethyl, cyclobutylmethyl, (oxetan-3-yl)methyl, (tetrahydrofuran-2-yl)methyl, (pyrrolidin-1-yl)methyl, benzyl, (thiazol-4-yl)methyl; each is optionally substituted with 1, 2, 3, 4 or 5 substituents independently selected from R^{B1}. In some embodiments, R^{B} is

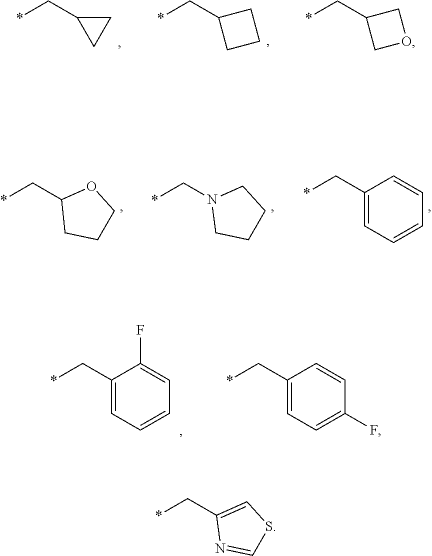

In some embodiments, R^{C} is H, D, C₁-C₆ alkyl optionally substituted with 1, 2, or 3 substituents independently selected from D, OH, CN, halo, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}.

In some embodiments, R^{C} is H, D, CH₃, CH₂CH₃, CD₃. In some embodiments, R^{C} is H. In some embodiments, R^{C} is D. In some embodiments, R^{C} is CH₃. In some embodiments, R^{C} is CH₂CH₃. In some embodiments, R^{C} is CD₃.

In some embodiments, R^{D} is H, D, C(O)R^{b}, C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, R^{D} is H. In some embodiments, R^{D} is D.

In some embodiments, R^{D} is C(O)R^{b}. In some embodiments, R^{D} is

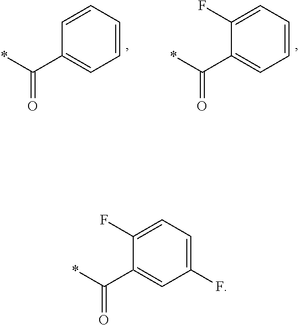 [SMILES: *C(=O)c1ccccc1 ; *C(=O)c1ccccc1F ; *C(=O)c1cc(F)ccc1F]

In some embodiments, R^{D} is C₁-C₆ alkyl optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}). In some embodiments, R^{D} is CH₃, CH₂CH₃, CH₂CH₂CH₃, CH(CH₃)₂, CH₂CH₂OH.

In some embodiments, R^{D} is C₃-C₁₀ cycloalkyl (such as saturated C₃-C₁₀ cycloalkyl or partially unsaturated C₃-C₁₀ cycloalkyl), 4-10 membered heterocycloalkyl (saturated 4-10 membered heterocycloalkyl or partially unsaturated 4-10 membered heterocycloalkyl); each is substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, R^{D} is cyclopropyl, cyclobutyl, cyclopentyl, azetidinyl, oxetanyl, pyrrolidinyl, tetrahydrofuranyl, piperidinyl, morpholinyl, quinazolin-4(1H)-onyl; each is 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{C}S(O)₂R^{b}, S(O)₂NR^{c}R^{d}, NR^{c}S(O)₂NR^{c}R^{d}, or B(OR^{e})(OR^{f}).

In some embodiments, R^{D} is C₆-C₁₀ aryl, 5-10 membered heteroaryl; each is 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{C}S(O)₂R^{b}, S(O)₂NR^{c}R^{d}, NR^{C}S(O)₂NR^{c}R^{d}, or B(OR^{e})(OR^{f}).

In some embodiments, R^{D} is phenyl, naphthalenyl, pyrrolyl, furanyl, thiophenyl, imidazolyl, oxazolyl, isoxazolyl, thiazolyl, pyrazolyl, thiadiazolyl, oxadiazolyl, pyridinyl, pyrimidinyl, pyrazinyl, pyridazinyl, indolyl, isoindolyl, indolizinyl, benzofuranyl, isobenzofuranyl, benzo[b]thiophenyl, benzo[c]thiophenyl, indazolyl, benzo[d]imidazolyl, pyrrolo[1,2-b]pyridazinyl, pyrrolo[3,2-b]pyridinyl, pyrrolo[2,3-b]pyridinyl, benzo[d]isoxazolyl, benzo[d]oxazolyl, furo[3,2-b]pyridinyl, pyrido[3,2-d]pyrimidinyl, 7H-pyrrolo[2,3-d]pyrimidinyl; each is optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR^{f}).

In some embodiments, R^{D} is

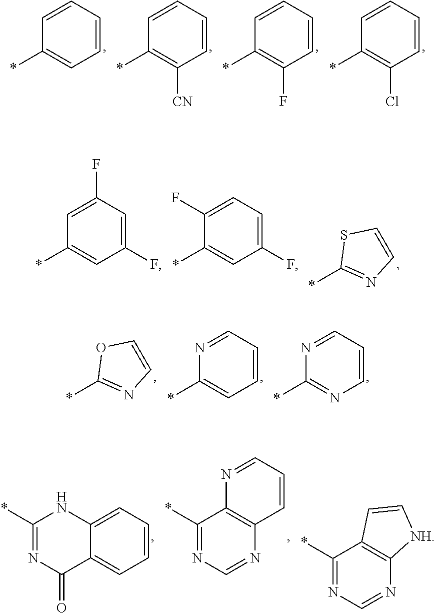

In some embodiments, R^{D} is C₆-C₁₀ aryl-C₁-C₂ alkyl, 5-10 membered heteroaryl-C₁-C₂ alkyl, C₃-C₁₀ cycloalkyl-C₁-C₂ alkyl, or 4-10 membered heterocycloalkyl-C₁-C₂ alkyl; each is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR).

In some embodiments, R^{D} is cyclopropylmethyl, cyclobutylmethyl, (oxetan-3-yl)methyl, (tetrahydrofuran-2-yl)methyl, (pyrrolidin-1-yl)methyl, benzyl, (thiazol-4-yl)methyl; each is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, R^{D} is

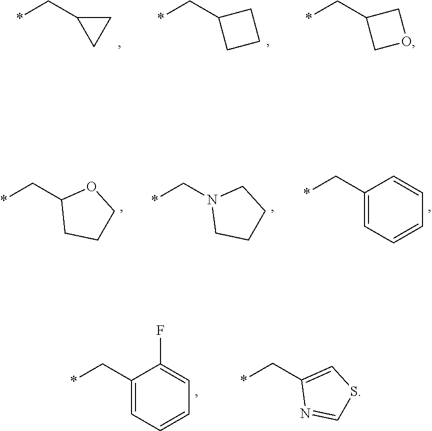

In some embodiments, Cy¹ is C₃-C₁₀ cycloalkyl, 4-10 membered heterocycloalkyl, C₆-C₁₀ aryl or 5-10 membered heteroaryl; wherein, the C₃-C₁₀ cycloalkyl, 4-10 membered heterocycloalkyl, C₆-C₁₀ aryl or 5-10 membered heteroaryl is optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is C₃-C₁₀ cycloalkyl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is 4-10 membered heterocycloalkyl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is

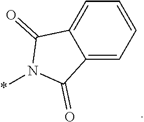 [SMILES: *N1C(=O)c2ccccc2C1=O]

In some embodiments, Cy¹ is C₆-C₁₀ aryl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is 5-10 membered heteroaryl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, the compounds of Formula (I) are represented by compounds of Formula (IIk):

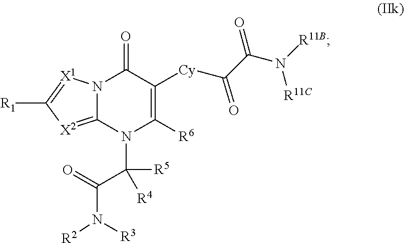

a pharmaceutically acceptable salt, stereoisomer, solvate, tautomer, isotopic variant, prodrug, N-oxide or deuterated compound thereof;
wherein, Cy, X¹, X², R¹, R², R³, R⁴, R⁵, R⁶, R^{11B} and R^{11C} are defined with respect to Formula (I).

In some embodiments, R^{11B} is H, D, OR^{A}, C(O)R^{B}, C₁-C₈ alkyl, C₂-C₈ alkenyl, C₂-C₈ alkynyl, C₃-C₁₀cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³.

In some embodiments, R^{11B} is H, D, OR^{A}, C₁-C₈ alkyl, C₃-C₁₀ cycloalkyl, 4-10 membered heterocycloalkyl, C₆-C₁₀ aryl, 5-10 membered heteroaryl; wherein, the C₁-C₈ alkyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³.

In some embodiments, R^{11B} is H, D, methyl, CH₂CH₃, CH(CH₃)₂, CH₂CF₃, OCH₃, OCH₂CH₃,

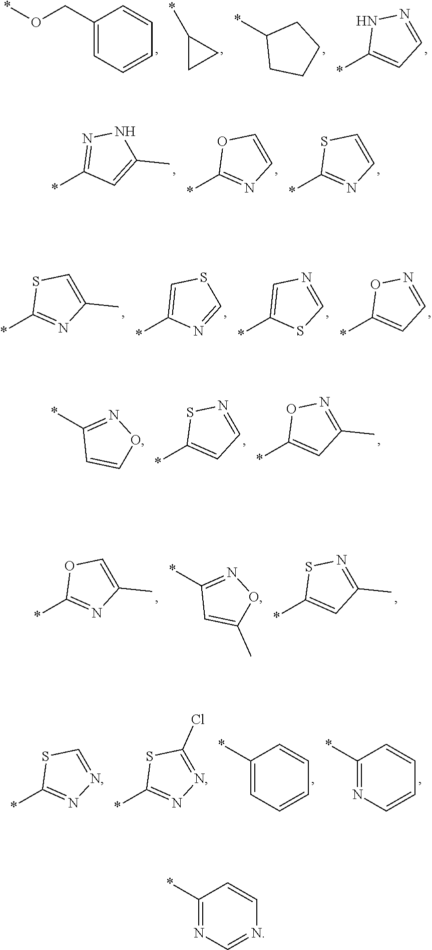

In some embodiments, R^{11C} is H, D, C₁-C₄ alkyl, C₂-C₄ alkenyl, C₂-C₄ alkynyl, C₃-C₇cycloalkyl, or 4-7 membered heterocycloalkyl; wherein, the C₁-C₄ alkyl, C₂-C₄ alkenyl, C₂-C₄ alkynyl, C₃-C₇ cycloalkyl or 4-7 membered heterocycloalkyl is optionally substituted by 1, 2, 3, 4, 5 or 6 substituents independently selected from D, halo, OH, oxo, CN, NO₂, N₃, C₁-C₄ alkyl, C₁-C₄ haloalkyl, OC₁-C₄ alkyl, or OC₁-C₄ haloalkyl;

In some embodiments, R^{11C} is H, D, C₁-C₄ alkyl optionally substituted by 1, 2, 3, 4, 5 or 6 substituents independently selected from D, halo, OH, oxo, CN, NO₂, N₃, C₁-C₄ alkyl, C₁-C₄ haloalkyl, OC₁-C₄ alkyl, or OC₁-C₄ haloalkyl.

In some embodiments, R^{11C} is H, D, methyl, CH₂CH₃.

In some embodiments, R^{11B} and R^{11C} together with the atom to which they are attached form a 4-10 membered heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, C₃-C₅ cycloalkyl or 4-5 membered heterocycloalkyl

In some embodiments, R^{11B} and R^{11C} together with the atoms to which they are attached form

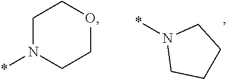 [SMILES: *N1CCOCC1 ; *N1CCCC1 ; ","]

In some embodiments, the compounds of Formula (IIb) are represented by compounds of Formula (V):

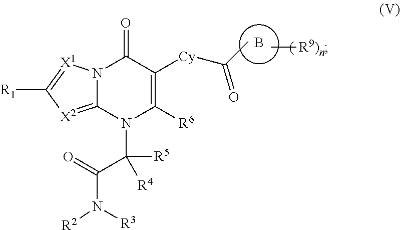

a pharmaceutically acceptable salt, stereoisomer, solvate, tautomer, isotopic variant, prodrug, N-oxide or deuterated compound thereof;
ring B is C₃-C₁₀ cycloalkyl, 4-10 membered heterocycloalkyl, C₆-C₁₀ aryl or 5-10 membered heteroaryl;
n is 1, 2, 3, 4, 5 or 6;
wherein, Cy, X¹, X², R¹, R², R³, R⁴, R⁵, R⁶ and R⁹ are defined with respect to Formula (I);
when

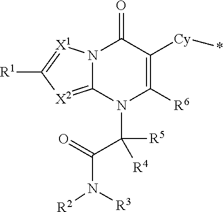

is

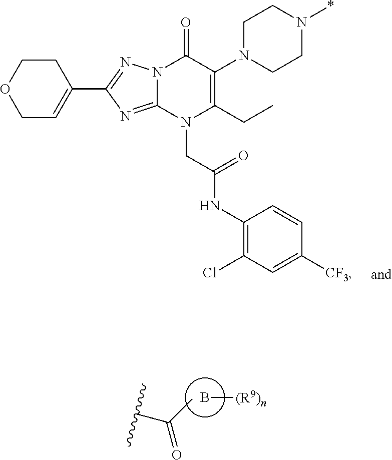

is not

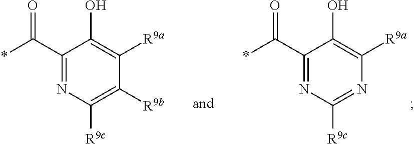

wherein, R^{9a} is H, F, Cl, or CH₃, OCF₃; R^{9b} is H, Cl, or CH₃; R^{9c} is H, or CH₃.

In some embodiments, ring B is C₃-C₁₀ cycloalkyl. In some embodiments, ring B is cyclopropyl, cyclobutyl, cyclopentyl.

In some embodiments, ring B is 4-10 membered heterocycloalkyl. In some embodiments, ring B is azetidinyl, oxetanyl, thietanyl, pyrrolidinyl, tetrahydrofuranyl, tetrahydrothiophenyl, piperidinyl, piperazinyl, morpholinyl, 2H-pyran-2-onyl, pyrrolidin-2-onyl.

In some embodiments, ring B is C₆-C₁₀ aryl. In some embodiments, ring B is phenyl, naphthalenyl.

In some embodiments, ring B is 5-membered heteroaryl. In some embodiments, ring B is 6-membered heteroaryl. In some embodiments, ring B is 7-membered heteroaryl. In some embodiments, ring B is 8-membered heteroaryl. In some embodiments, ring B is 9-membered heteroaryl. In some embodiments, ring B is 10-membered heteroaryl.

In some embodiments, ring B is pyrrolyl, furanyl, thiophenyl, imidazolyl, oxazolyl, thiazolyl, tetrazolyl, pyrazolyl, 1,2,4-triazolyl, 1,2,3-triazolyl, thiadiazolyl, oxadiazolyl, pyridinyl, pyrimidinyl, pyrazinyl, pyridazinyl, indolyl, isoindolyl, indazolyl, indolizinyl, purinyl, benzofuranyl, isobenzofuranyl, benzo[b]thiophenyl, benzo[c]thiophenyl, indazolyl, benzo[d]imidazolyl, pyrrolo[3,2-b]pyridinyl, 1H-pyrrolo[3,2-c]pyridinyl, 1H-pyrrolo[2,3-c]pyridinyl, 1H-pyrrolo[2,3-b]pyridinyl, pyrrolo[3,4-b]pyridinyl, pyrrolo[3,4-c]pyridinyl, benzo[d]isoxazolyl, benzo[d]oxazolyl, furo[3,2-b]pyridinyl, furo[3,2-c]pyridinyl, furo[2,3-c]pyridinyl, furo[2,3-b]pyridinyl, benzo[c]isoxazolyl, furo[3,4-b]pyridinyl, furo[3,4-c]pyridinyl, benzo[d]isothiazolyl, benzo[d]thiazolyl, thieno[2,3-b]pyridinyl, thieno[3,2-b]pyridinyl, thieno[3,4-c]pyridinyl, benzo[d][1,2,3]triazolyl, pyrrolo[1,2-c]pyrimidinyl, pyrazolo[1,5-c]pyrimidinyl, pyrazolo[4,3-b]pyridinyl, pyrazolo[4,3-c]pyridinyl, pyrazolo[3,4-c]pyridinyl, 1H-pyrazolo[3,4-b]pyridinyl, pyrrolo[1,2-b]pyridazinyl, pyrazolo[1,5-a]pyridinyl, pyrrolo[1,2-a]pyrimidinyl, imidazo[1,5-b]pyridazinyl, thieno[3,2-b]pyridin-5(4H)-onyl, 1,6-naphthyridinyl, 1,5-naphthyridinyl, thieno[3,2-b]pyridinyl, quinolinyl, isoquinolinyl, thieno[2,3-b]pyridin-6 (7H)-onyl, thiazolo[4,5-c]pyridinyl, 1H-pyrrolo[2,3-b]pyridinyl, 1H-pyrrolo[3,2-b]pyridinyl, 1H-pyrazolo[3,4-d]pyrimidinyl, thieno[3,2-c]pyridinyl, 5H-pyrrolo[3,2-d]pyrimidinyl, 1H-pyrazolo[4,3-d]pyrimidinyl, 7H-pyrrolo[2,3-d]pyrimidinyl, [1,2,4]triazolo[1,5-a]pyrimidinyl, imidazo[1,2-b]pyridazinyl, thieno[2,3-b]pyridinyl, thiazolo[4,5-b]pyridinyl, pyrimidin-2(1H)-onyl, 1,2,4-triazin-5(2H)-onyl, pyrazin-2(1H)-onyl, pyridazin-3(2H)-onyl, pyrimidine-2,4(1H,3H)-dionyl, 1,2,4-triazine-3,5(2H,4H)-dionyl, 1,2-dihydro-3H-pyrazol-3-onyl, pyridazin-4(1H)-onyl, pyridin-2(1H)-onyl, imidazo[1,2-c]pyrimidinyl, thieno[2,3-c]pyridinyl, 3H-imidazo[4,5-b]pyridinyl, 3H-imidazo[4,5-c]pyridinyl, imidazo[1,2-a]pyrazinyl, imidazo[4,5-b]pyridinyl, pyrrolo[3,2-c]pyridazinyl, pyrrolo[3,2-d]pyrimidinyl, pyrrolo[2,3-b]pyrazinyl, pyrrolo[2,3-d]pyridazinyl, pyrrolo[2,3-d]pyrimidinyl, pyrrolo[2,3-c]pyridazinyl, pyrrolo[3,4-c]pyridazinyl, pyrrolo[3,4-d]pyrimidinyl, pyrrolo[3,4-b]pyrazinyl, pyrrolo[3,4-d]pyridazinyl, pyrrolo[3,4-d]pyrimidinyl, 6H-pyrrolo[3,4-c]pyridazinyl, thiazolo[5,4-b]pyridinyl.

In some embodiments, each R⁹ is independently H, D, halo, CN, NO₂, N₃, oxo, C₁-C₆ alkyl, C₂-C₆ alkenyl, C₂-C₆ alkynyl, —NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁹ is independently H, D, halo, CN, NO₂, N₃, oxo, C₁-C₆ alkyl, C₂-C₆ alkenyl, C₂-C₆ alkynyl, —NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, S(O)R^{B}, S(O)NR^{C}R^{D}, S(O)₂R^{B}, S(O)₂NR^{C}R^{D}, NR^{C}S(O)₂R^{B}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁹ is independently H, D, halo, CN, NO₂, N₃, oxo. In some embodiments, each R⁹ is independently H. In some embodiments, each R⁹ is independently D. In some embodiments, each R⁹ is independently halo (such as F, Cl, Br or I). In some embodiments, each R⁹ is independently CN. In some embodiments, each R⁹ is independently NO₂. In some embodiments, each R⁹ is independently N₃. In some embodiments, each R⁹ is independently oxo.

In some embodiments, each R⁹ is independently C₁-C₆ alkyl optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl. In some embodiments, each R⁹ is independently CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN.

In some embodiments, each R⁹ is independently C₂-C₆ alkenyl optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁹ is independently C₂-C₆ alkynyl optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁹ is independently C₁-C₄ alkyl-O—C₁-C₄ alkyl-Si(C₁-C₄ alkyl)₃. In some embodiments, each R⁹ is independently-NR^{C}R^{D}. In some embodiments, each R⁹ is independently NH₂, NHCH₃, N(CH₃)₂, NHCH₂CH₃, N(CH₂CH₃)₂, NHCH₂CH₂CH₃, N(CH₂CH₂CH₃)₂, NHCH(CH₃)₂, NHCH₂CH₂OH, N(CH₃)CH₂CH₂OH, NHCH₂CH₂OCH₃, N(CH₃)CH₂CH₂OCH₃.

In some embodiments, each R⁹ is independently OR^{A}. In some embodiments, each R⁹ is independently OH, OCH₃, OCH₂CH₃, OCH₂CH₂CH₃, OCH(CH₃)₂, OCH₂F, OCHF₂, OCF₃, OCH₂CH₂F, OCH₂CHF₂, OCH₂CF₃, OCF₂CF₃, OCH₂CH₂OH, OCH₂CH₂OCH₃.

In some embodiments, each R⁹ is independently SR^{A}.

In some embodiments, each R⁹ is independently C(O)R^{B}. In some embodiments, each R⁹ is independently C(O)OR^{A}. In some embodiments, each R⁹ is independently OC(O)R^{B}. In some embodiments, each R⁹ is independently C(O)NR^{C}R^{D}. In some embodiments, each R⁹ is independently NR^{C}C(O)R^{B}. In some embodiments, each R⁹ is independently OC(O)NR^{C}R^{D}. In some embodiments, each R⁹ is independently OC(O)OR^{A}.

In some embodiments, each R⁹ is independently NR^{c}C(O)NR^{C}R^{D}. In some embodiments, each R⁹ is independently NR^{C}C(O)OR^{A}. In some embodiments, each R⁹ is independently C(═NR^{C})NR^{C}R^{D}. In some embodiments, each R⁹ is independently NR^{D}C(═NR^{C})NR^{C}R^{D}. In some embodiments, each R⁹ is independently NR^{D}C(═NR^{C})R^{B}.

In some embodiments, each R⁹ is independently S(O)R^{B}. In some embodiments, each R⁹ is independently S(O)NR^{C}R^{D}. In some embodiments, each R⁹ is independently S(O)₂R^{B}. In some embodiments, each R⁹ is independently S(O)₂NR^{C}R^{D}. In some embodiments, each R⁹ is independently NR^{C}S(O)₂R^{B}. In some embodiments, each R⁹ is independently S(O)(═NR^{B})R^{B}. In some embodiments, each R⁹ is independently NR^{C}S(O)₂NR^{C}R^{D}. In some embodiments, each R⁹ is independently NR^{C}S(O)(═NR^{B})R^{B}.

In some embodiments, each R⁹ is independently B(OR^{E})(OR^{F}). In some embodiments, each R⁹ is independently P(O)R^{E}R^{F}. In some embodiments, each R⁹ is independently P(O)OR^{E}OR^{F}. In some embodiments, each R⁹ is independently OP(O)OR^{E}OR^{F}.

In some embodiments, each R⁹ is independently Cy¹, and Cy¹ is C₆-C₁₀ aryl or 5-10 membered heteroaryl; wherein, each is optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}. In some embodiments, each R⁹ is independently 2-fluoro-phenyl, or 5-fluoro-2-methoxy-4-pyridinyl.

In some embodiments, each R⁹ is independently H, D, halo, CN, oxo, C₁-C₆ alkyl, —NR^{C}R^{D}, OR^{A}, C(O)OR^{A}, or Cy¹; wherein, the C₁-C₆ alkyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl.

In some embodiments, each R⁹ is independently H, D, halo, CN, oxo, CH₃, CHF₂, CF₃, —NH₂, OH, OCH₃, COOH, COOCH₂CH₃, 2-fluoro-phenyl, or 5-fluoro-2-methoxy-4-pyridinyl.

In some embodiments, Cy¹ is C₃-C₁₀ cycloalkyl, 4-10 membered heterocycloalkyl, C₆-C₁₀ aryl or 5-10 membered heteroaryl; wherein, the C₃-C₁₀ cycloalkyl, 4-10 membered heterocycloalkyl, C₆-C₁₀ aryl or 5-10 membered heteroaryl is optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is C₃-C₁₀ cycloalkyl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is 4-10 membered heterocycloalkyl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{D}, NR^{c}S(O)₂R^{b}, or S(O)₂NR^{c}R^{d}.

In some embodiments, Cy¹ is C₆-C₁₀ aryl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, Cy¹ is 5-10 membered heteroaryl optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, the moiety

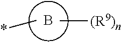

has the structure of

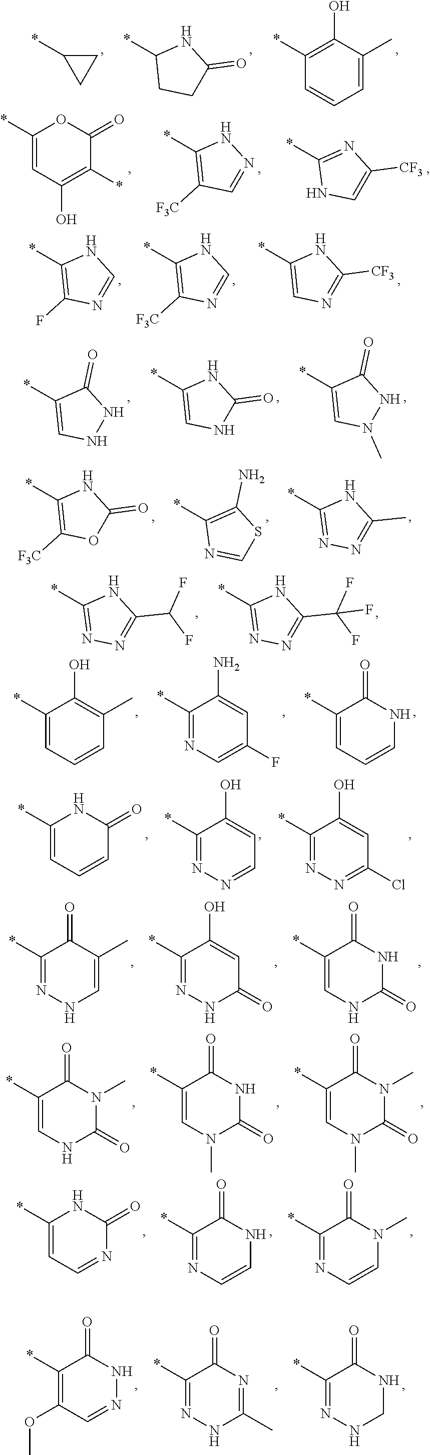

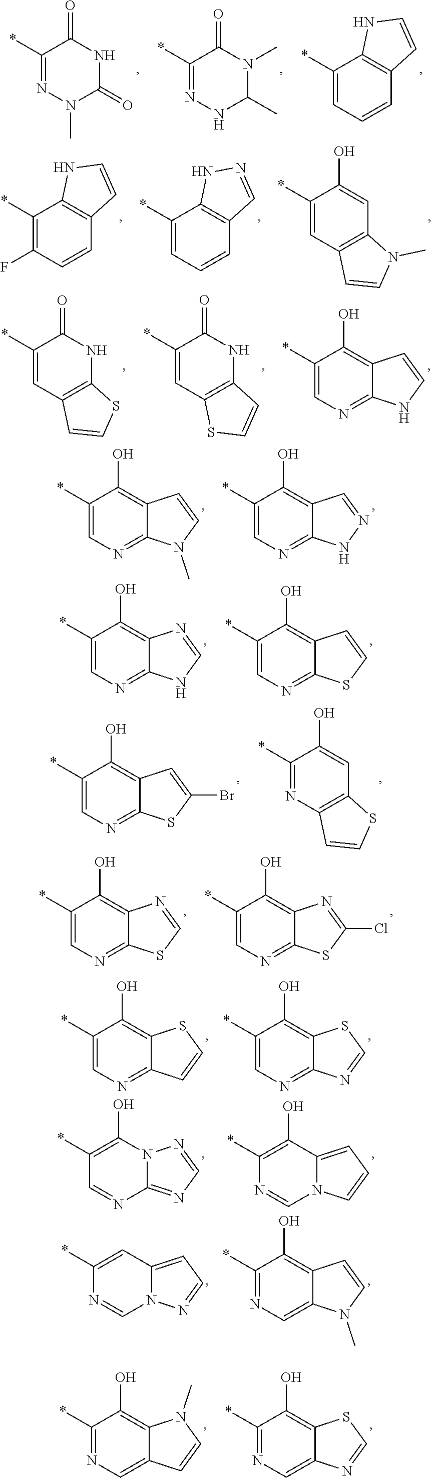

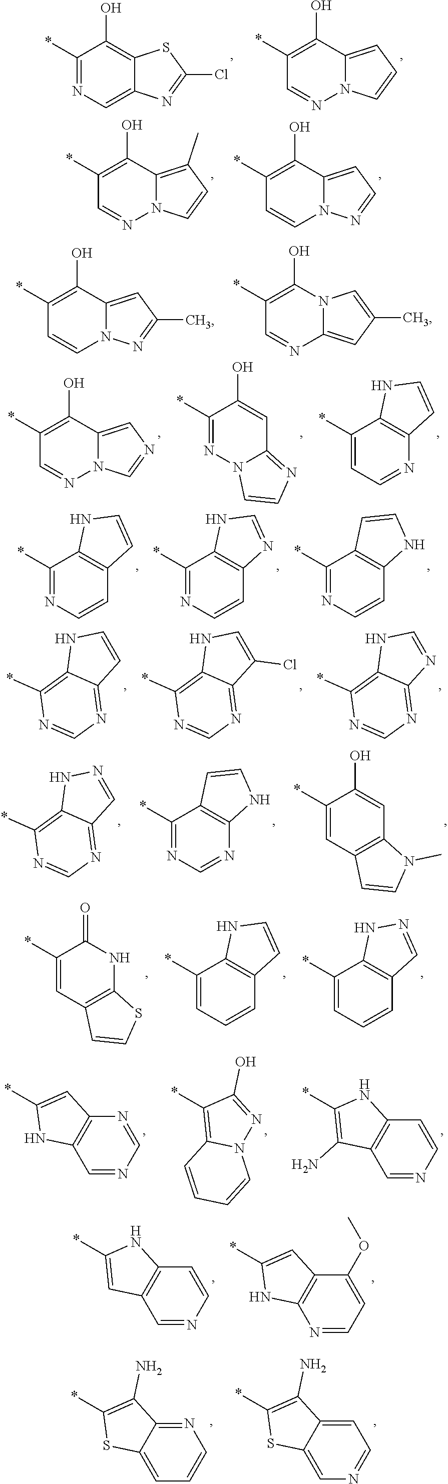

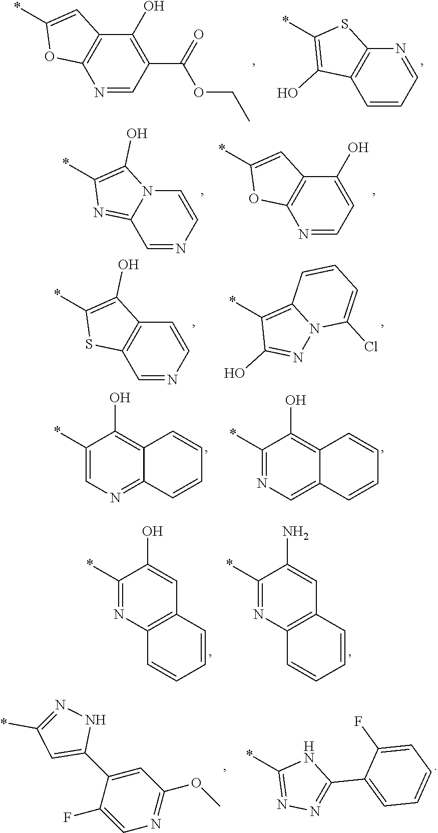

In some embodiments, each R¹⁰ is independently H, D, CN, OH, OMe, or C₁-C₄ alkyl optionally substituted by 1, 2, 3, 4 or 5 substituents independently selected from D, halo, CN, —OH, —O—C₁-C₄ alkyl, —OC₁-C₄ haloalkyl, NH₂, —NH(C₁-C₄ alkyl), or —N(C₁-C₄ alkyl)₂.

In some embodiments, each R¹⁰ is independently H, D, CN, OH, OMe. In some embodiments, R¹⁰ is independently H. In some embodiments, R¹⁰ is independently D. In some embodiments, R¹⁰ is independently CN. In some embodiments, R¹⁰ is independently OH. In some embodiments, R¹⁰ is independently OMe.

In some embodiments, R¹⁰ is independently C₁-C₄ alkyl optionally substituted by 1, 2, 3, 4 or 5 substituents independently selected from D, halo, CN, —OH, —O—C₁-C₄ alkyl, —OC₁-C₄ haloalkyl, NH₂, —NH(C₁-C₄ alkyl), or —N(C₁-C₄ alkyl)₂. In some embodiments, R¹⁰ is independently CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN.

In some embodiments, each R¹¹ is independently C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³.

In some embodiments, each R¹¹ is independently C₁-C₈ alkyl optionally substituted by 1, 2, 3, 4, 5 or 6 substituents independently selected from R¹³.

In some embodiments, each R¹¹ is independently C₂-C₈ alkenyl optionally substituted by 1, 2, 3, 4, 5 or 6 substituents independently selected from R¹³.

In some embodiments, each R¹¹ is independently C₂-C₈ alkynyl optionally substituted by 1, 2, 3, 4, 5 or 6 substituents independently selected from R¹³.

In some embodiments, each R¹¹ is independently C₃-C₁₀ cycloalkyl optionally substituted by 1, 2, 3, 4, 5 or 6 substituents independently selected from R¹³.

In some embodiments, each R¹¹ is independently 4-10 membered heterocycloalkyl optionally substituted by 1, 2, 3, 4, 5 or 6 substituents independently selected from R¹³.

In some embodiments, each R¹¹ is independently C₆-C₁₀ aryl optionally substituted by 1, 2, 3, 4, 5 or 6 substituents independently selected from R¹³.

In some embodiments, each R¹¹ is independently 5-10 membered heteroaryl optionally substituted by 1, 2, 3, 4, 5 or 6 substituents independently selected from R¹³.

In some embodiments, R¹⁰ and R¹¹ together with the atoms to which they are attached form 5-6 membered heteroaryl, 5-6 membered partially unsaturated heterocycloalkyl; wherein, the 5-6 membered heteroaryl or 5-6 membered partially unsaturated heterocycloalkyl is optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, or SC₁-C₄ alkyl.

In some embodiments, R¹⁰ and R¹¹ together with the atoms to which they are attached form 5-6 membered heteroaryl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, or SC₁-C₄ alkyl.

In some embodiments, R¹⁰ and R¹¹ together with the atoms to which they are attached form 5-6 membered partially unsaturated heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, or SC₁-C₄ alkyl.

In some embodiments, each RIA is independently H, D, C₁-C₈ alkyl, C₂-C₈ alkenyl, C₂-C₈ alkynyl, C₃-C₁₀cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³.

In some embodiments, each R^{11A} is independently H. In some embodiments, each R¹¹ is independently D.

In some embodiments, each R^{11A} is independently C₁-C₈ alkyl optionally substituted by 1, 2, 3, 4, 5 or 6 substituents independently selected from R¹³.

In some embodiments, each R^{11A} is independently C₂-C₈ alkenyl optionally substituted by 1, 2, 3, 4, 5 or 6 substituents independently selected from R¹³.

In some embodiments, each R^{11A} is independently C₂-C₈ alkynyl optionally substituted by 1, 2, 3, 4, 5 or 6 substituents independently selected from R¹³.

In some embodiments, each RIA is independently C₃-C₁₀ cycloalkyl optionally substituted by 1, 2, 3, 4, 5 or 6 substituents independently selected from R¹³.

In some embodiments, each R^{11A} is independently 4-10 membered heterocycloalkyl optionally substituted by 1, 2, 3, 4, 5 or 6 substituents independently selected from R¹³.

In some embodiments, each R^{11A} is independently C₆-C₁₀ aryl optionally substituted by 1, 2, 3, 4, 5 or 6 substituents independently selected from R¹³.

In some embodiments, each R^{11A} is independently 5-10 membered heteroaryl optionally substituted by 1, 2, 3, 4, 5 or 6 substituents independently selected from R¹³.

In some embodiments, each R¹³ is independently H, D, halo, CN, NO₂, N₃, oxo, SF₅, C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl, C₀-C₆ alkyl-5-10 membered heteroaryl, C₁-C₄ alkyl-O—C₁-C₄ alkyl-Si(C₁-C₄ alkyl)₃, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{c}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, or OP(O)OR^{E}OR^{F}; wherein, the C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl or C₀-C₆ alkyl-5-10 membered heteroaryl is optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently H, D, halo, CN, NO₂, N₃, oxo, SF₅. In some embodiments, each R¹³ is independently H. In some embodiments, each R¹³ is independently D. In some embodiments, each R¹³ is independently halo (such as F, Cl, Br or I). In some embodiments, each R¹³ is independently CN. In some embodiments, each R¹³ is independently NO₂. In some embodiments, each R¹³ is independently N₃. In some embodiments, each R¹³ is independently oxo. In some embodiments, each R¹³ is independently SF₅.

In some embodiments, each R¹³ is independently C₁-C₆ alkyl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently C₂-C₆ alkenyl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently C₂-C₆ alkynyl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently C₀-C₆ alkyl-C₃-C₆ cycloalkyl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{C}S(O)₂R^{b}, or S(O)₂NR^{c}R^{d}.

In some embodiments, each R¹³ is independently C₀-C₆ alkyl-4-6 membered heterocycloalkyl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently C₀-C₆ alkyl-C₆-C₁₀ aryl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d}.

In some embodiments, each R¹³ is independently C₀-C₆ alkyl-5-10 membered heteroaryl optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{C}S(O)₂R^{b}, or S(O)₂NR^{c}R^{d}.

In some embodiments, each R¹³ is independently C₁-C₄ alkyl-O—C₁-C₄ alkyl-Si(C₁-C₄ alkyl)₃.

In some embodiments, each R¹³ is independently NR^{C}R^{D}. In some embodiments, each R¹³ is independently OR^{A}. In some embodiments, each R¹³ is independently SR^{A}.

In some embodiments, each R¹³ is independently C(O)R^{B}. In some embodiments, each R¹³ is independently C(O)OR^{A}. In some embodiments, each R¹³ is independently OC(O)R^{B}. In some embodiments, each R¹³ is independently C(O)NR^{C}R^{D}. In some embodiments, each R¹³ is independently NR^{C}C(O)R^{B}. In some embodiments, each R¹³ is independently OC(O)NR^{C}R^{D}. In some embodiments, each R¹³ is independently OC(O)OR^{A}.

In some embodiments, each R¹³ is independently NR^{c}C(O)NR^{C}R^{D}. In some embodiments, each R¹³ is independently NR^{c}C(O)OR^{A}. In some embodiments, each R¹³ is independently C(═NR^{C})NR^{C}R^{D}. In some embodiments, each R¹³ is independently NR^{D}C(═NR^{C})NR^{C}R^{D}. In some embodiments, each R¹³ is independently NR^{D}C(═NR^{C})R^{B}.

In some embodiments, each R¹³ is independently S(O)R^{B}. In some embodiments, each R¹³ is independently S(O)NR^{C}R^{D}. In some embodiments, each R¹³ is independently S(O)₂R^{B}. In some embodiments, each R¹³ is independently S(O)₂NR^{C}R^{D}. In some embodiments, each R¹³ is independently NR^{C}S(O)₂R^{B}. In some embodiments, each R¹³ is independently S(O)(═NR^{B})R^{B}. In some embodiments, each R¹³ is independently NR^{C}S(O)₂NR^{C}R^{D}. In some embodiments, each R¹³ is independently NR^{C}S(O)(═NR^{B})R^{B}.

In some embodiments, each R¹³ is independently B(OR^{E})(OR^{F}). In some embodiments, each R¹³ is independently P(O)R^{E}R^{F}. In some embodiments, each R¹³ is independently P(O)OR^{E}OR^{F}. In some embodiments, each R¹³ is independently OP(O)OR^{E}OR^{F}.

In some embodiments, R^{A} is independently H, D, C₁-C₆ alkyl, C₂-C₆ alkenyl, C₂-C₆ alkynyl, C₃-C₁₀cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, C₁-C₄ alkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{c}OR^{d}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or NR^{c}S(O)(═NR^{b})R^{b}.

In some embodiments, R^{A} is independently H, D. In some embodiments, R^{A} is independently H.

In some embodiments, R^{A} is independently D.

In some embodiments, R^{A} is independently C₁-C₆ alkyl, C₂-C₆ alkenyl or C₂-C₆ alkynyl, wherein the C₁-C₆ alkyl, C₂-C₆ alkenyl or C₂-C₆ alkynyl is optionally substituted with 1, 2, 3, 4 or 5 substituents independently selected from D, OH, CN, halo, C₁-C₄ alkyl, C₃-C₆ cycloalkyl, 4-6 membered heterocycloalkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{c})NR^{c}R^{d}, NR^{d}C(═NR^{c})NR^{c}R^{d}, NR^{d}C(═NR^{C})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, NR^{C}S(O)(═NR^{b})R^{b}.

In other embodiments, R^{A} is independently C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, C₁-C₄ alkyl, C₃-C₆ cycloalkyl, 4-6 membered heterocycloalkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{c})NR^{c}R^{d}, NR^{d}C(═NR^{c})NR^{c}R^{d}, NR^{d}C(═NR^{C})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, NR^{C}S(O)(═NR^{b})R^{b}.

In some embodiments, each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, each R^{B} is independently H, D. In some embodiments, each R^{B} is independently H. In some embodiments, each R^{B} is independently D.

In some embodiments, each R^{B} is independently C₁-C₆ alkyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR^{f}).

In some embodiments, each R^{B} is independently C₂-C₆ alkynyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR^{f}).

In some embodiments, each R^{B} is independently C₂-C₆ alkenyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, each R^{B} is independently C₃-C₁₀ cycloalkyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{C}S(O)₂R^{b}, S(O)₂NR^{c}R^{d}, NR^{C}S(O)₂NR^{c}R^{d}, or B(OR^{e})(OR^{f}).

In some embodiments, each R^{B} is independently C₃-C₁₀ cycloalkyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, C₁-C₄ alkyl-O—C₁-C₄ alkyl, C₁-C₄ alkyl-O—C₁-C₄ haloalkyl, OC₂-C₄ alkylOH, OC₂-C₄ alkyl-O—C₁-C₄ alkyl, OC₂-C₄ alkyl-O—C₁-C₄ haloalkyl.

In some embodiments, each R^{B} is independently cyclopropyl optionally substituted with 1, 2, 3, 4 or 5 substituents independently selected from D, OH, CN, halo, C₁-C₄ alkyl, C₁-C₄ haloalkyl, C₁-C₄ cyanoalkyl, OC₁-C₄ alkyl, OC₁-C₄ haloalkyl, C₁-C₄ alkyl-O—C₁-C₄ alkyl, C₁-C₄ alkyl-O—C₁-C₄ haloalkyl.

In some embodiments, each R^{B} is independently cyclobutyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, C₁-C₄ alkyl-O—C₁-C₄ alkyl, C₁-C₄ alkyl-O—C₁-C₄ haloalkyl, OC₂-C₄ alkylOH, OC₂-C₄ alkyl-O—C₁-C₄ alkyl, OC₂-C₄ alkyl-O—C₁-C₄ haloalkyl.

In some embodiments, each R^{B} is independently cyclopentyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, C₁-C₄ alkyl-O—C₁-C₄ alkyl, C₁-C₄ alkyl-O—C₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR^{f}).

In some embodiments, each R^{B} is independently cyclohexyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, C₁-C₄ alkyl-O—C₁-C₄ alkyl, C₁-C₄ alkyl-O—C₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In other embodiments, each R^{B} is independently 4-10 membered heterocycloalkyl optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, each R^{B} is independently azetidinyl, pyrrolidinyl, piperidinyl or azepanyl; each ring is optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄alkyl-O—C₁-C₄ alkyl, OC₂-C₄ alkyl-O—C₁-C₄ haloalkyl, C₁-C₄ alkyl-O—C₁-C₄alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In other embodiments, each R^{B} is independently C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or B(OR^{e})(OR^{f}).

In some embodiments, R^{C} and R^{D} are each independently H, D, C₁-C₆ alkyl, C₂-C₆ alkenyl, C₂-C₆ alkynyl, C₃-C₇cycloalkyl, 4-7 membered heterocycloalkyl, phenyl, 5-6 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, phenyl, 5-6 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, each R^{C} is independently H, D. In some embodiments, each R^{C} is independently H. In some embodiments, each R^{C} is independently D.

In some embodiments, each R^{C} is independently C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In some embodiments, each R^{D} is independently H, D. In some embodiments, each R^{D} is independently H. In some embodiments, each R^{D} is independently D.

In some embodiments, each R^{D} is independently C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{c}S(O)₂R^{b}, S(O)₂NR^{c}R^{d}, NR^{C}S(O)₂NR^{c}R^{d}, or B(OR^{e})(OR^{f}).

In other embodiments, R^{C} and R^{D} together with the N atom to which they are attached form 4-7 membered heterocycloalkyl optionally substituted with 1, 2, or 3 substituents independently selected from D, OH, oxo, CN, —NH₂, —NH(C₁-C₄ alkyl), —N(C₁-C₄ alkyl)₂, halo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, or OC₂-C₄ alkyl-O—C₁-C₄ haloalkyl.

In some embodiments, each R^{F} is independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄alkyl.

In some embodiments, each R^{E} is independently H, or D. In some embodiments, each R^{E} is independently H. In some embodiments, each R^{E} is independently D.

In some embodiments, each R^{E} is independently C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄alkyl.

In some embodiments, each R^{F} is independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl.

In some embodiments, each R^{a} is independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy.

In some embodiments, each R^{a} is independently H, or D. In some embodiments, each R^{a} is independently H. In some embodiments, each R^{a} is independently D.

In some embodiments, each R^{a} is independently C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy.

In some embodiments, each R^{b} is independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₆-C₁₀ aryl, C₃-C₁₀ cycloalkyl, 5-10 membered heteroaryl, or 4-10 membered heterocycloalkyl.

In some embodiments, each R^{b} is independently H, D. In some embodiments, each R^{b} is independently H. In some embodiments, each R^{b} is independently D.

In some embodiments, each R^{b} is independently C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₆-C₁₀ aryl, C₃-C₁₀ cycloalkyl, 5-10 membered heteroaryl, or 4-10 membered heterocycloalkyl.

In some embodiments, each R^{e} is independently H, D, C₁-C₄ alkyl, C₁-C₄ haloalkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl); wherein the C₁-4 alkyl, C₂-4 alkenyl, C₂-4 alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl) is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁—C₄ cyanoalkyl, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—.

In some embodiments, each R^{c} is independently H, D. In some embodiments, each R^{c} is independently H. In some embodiments, each R^{c} is independently D.

In some embodiments, each R^{c} is independently C₁-C₄ alkyl, C₁-C₄ haloalkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀cycloalkyl, C₀-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl); wherein the C₁-4 alkyl, C₂-4 alkenyl, C₂₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl) is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—.

In some embodiments, each R^{d} is independently H, D. In some embodiments, each R^{d} is independently H. In some embodiments, each R^{d} is independently D.

In some embodiments, each R^{d} is independently C₁-C₄ alkyl, C₁-C₄ haloalkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₀-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl); wherein the C₁₄ alkyl, C₂-4 alkenyl, C₂-4 alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀cycloalkyl, 4-10 membered heteroaryl-4-10 membered heterocycloalkyl, 4-10 membered heteroaryl-C₆-C₁₀ aryl, or bi(4-10 membered heteroaryl) is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—.

In some embodiments, R^{c} and R^{d} together with the N atom to which they are attached form 4-7 membered heterocycloalkyl (such as 4-membered heterocycloalkyl, 5-membered heterocycloalkyl, 6-membered heterocycloalkyl, 7-membered heterocycloalkyl) optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy.

In some embodiments, each R^{e} is independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl.

In some embodiments, each R^{e} is independently H, D. In some embodiments, each R^{c} is independently H. In some embodiments, each R^{e} is independently D.

In some embodiments, each R^{e} is independently C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl.

In some embodiments, each R^{c} is independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl.

In some embodiments, each R^{f} is independently H. In some embodiments, each R^{f} is independently D. In some embodiments, each R^{f} is independently C₁-C₄ alkyl. In some embodiments, each R^{f} is independently C₂-C₄ alkenyl. In some embodiments, each R^{f} is independently C₂-C₄ alkynyl. In some embodiments, each R^{f} is independently C₆-C₁₀ aryl. In some embodiments, each R^{f} is independently 5-10 membered heteroaryl. In some embodiments, each R^{f} is independently C₃-C₁₀ cycloalkyl. In some embodiments, each R^{f} is independently 4-10 membered heterocycloalkyl.

In some embodiments, each R^{a1} is independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy.

In some embodiments, each R^{a1} is independently H, D. In some embodiments, each R^{a1} is independently H. In some embodiments, each R^{a1} is independently D.

In some embodiments, each R^{a1} is independently C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy.

In some embodiments, each R^{b1} is independently H, D, C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₀-C₁₀ aryl, C₃-C₁₀ cycloalkyl, 5-10 membered heteroaryl, or 4-10 membered heterocycloalkyl.

In some embodiments, each R^{b1} is independently H, D. In some embodiments, each R^{b1} is independently H. In some embodiments, each R^{b1} is independently D.

In some embodiments, each R^{b1} is independently C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₀-C₁₀ aryl-C₁—C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₆-C₁₀ aryl, C₃-C₁₀ cycloalkyl, 5-10 membered heteroaryl, or 4-10 membered heterocycloalkyl.

Stereoisomers of the compounds of Formula I, and the pharmaceutical salts and solvates thereof, are also contemplated, described, and encompassed herein. Methods of using compounds of Formula I are described, as well as pharmaceutical compositions including the compounds of Formula I.

In some embodiments, the compound of Formula (I) is:

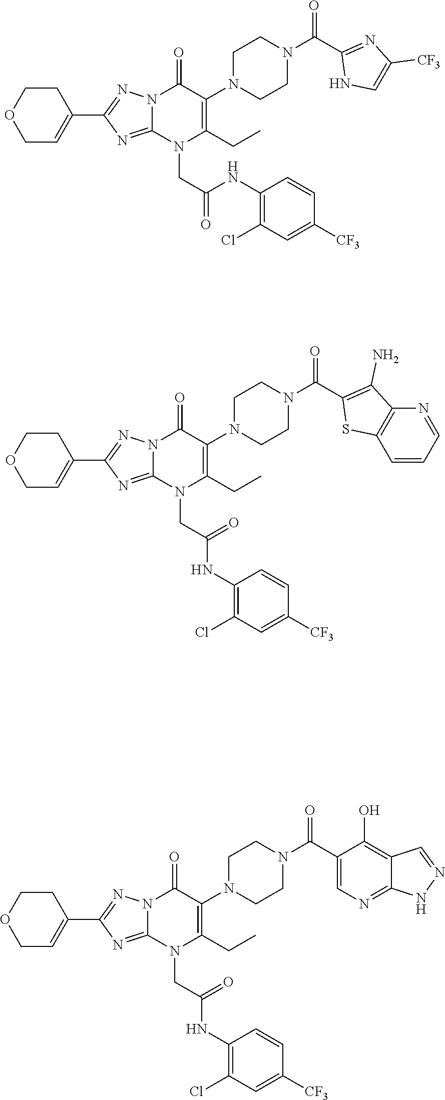 [SMILES: CCc1c(N2CCN(C(=O)c3nc(C(F)(F)F)c[nH]3)CC2)c(=O)n2nc(C3=CCOCC3)nc2n1CC(=O)Nc1ccc(C(F)(F)F)cc1Cl ; CCc1c(N2CCN(C(=O)c3sc4cccnc4c3N)CC2)c(=O)n2nc(C3=CCOCC3)nc2n1CC(=O)Nc1ccc(C(F)(F)F)cc1Cl ; CCc1c(N2CCN(C(=O)c3cnc4[nH]ncc4c3O)CC2)c(=O)n2nc(C3=CCOCC3)nc2n1CC(=O)Nc1ccc(C(F)(F)F)cc1Cl]

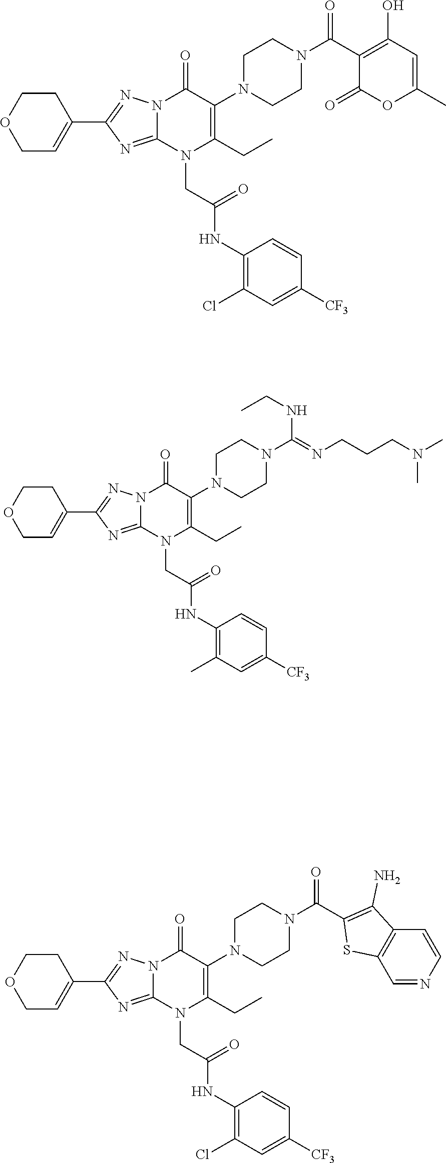 [SMILES: CCc1c(N2CCN(C(=O)c3c(O)cc(C)oc3=O)CC2)c(=O)n2nc(C3=CCOCC3)nc2n1CC(=O)Nc1ccc(C(F)(F)F)cc1Cl ; CCN/C(=N\CCCN(C)C)N1CCN(c2c(CC)n(CC(=O)Nc3ccc(C(F)(F)F)cc3C)c3nc(C4=CCOCC4)nn3c2=O)CC1 ; CCc1c(N2CCN(C(=O)c3sc4cnccc4c3N)CC2)c(=O)n2nc(C3=CCOCC3)nc2n1CC(=O)Nc1ccc(C(F)(F)F)cc1Cl]

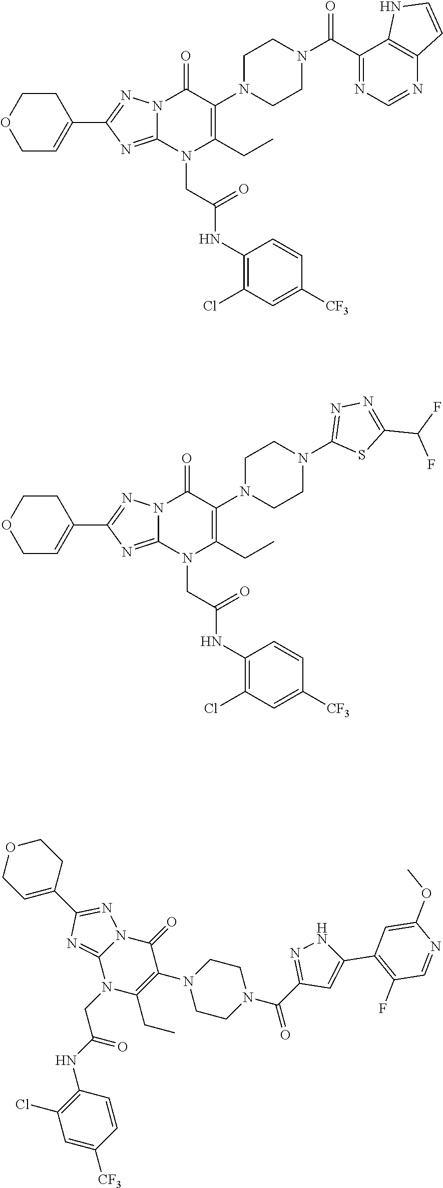 [SMILES: CCc1c(N2CCN(C(=O)c3ncnc4cc[nH]c34)CC2)c(=O)n2nc(C3=CCOCC3)nc2n1CC(=O)Nc1ccc(C(F)(F)F)cc1Cl ; CCc1c(N2CCN(c3nnc(C(F)F)s3)CC2)c(=O)n2nc(C3=CCOCC3)nc2n1CC(=O)Nc1ccc(C(F)(F)F)cc1Cl ; CCc1c(N2CCN(C(=O)c3cc(-c4cc(OC)ncc4F)[nH]n3)CC2)c(=O)n2nc(C3=CCOCC3)nc2n1CC(=O)Nc1ccc(C(F)(F)F)cc1Cl]

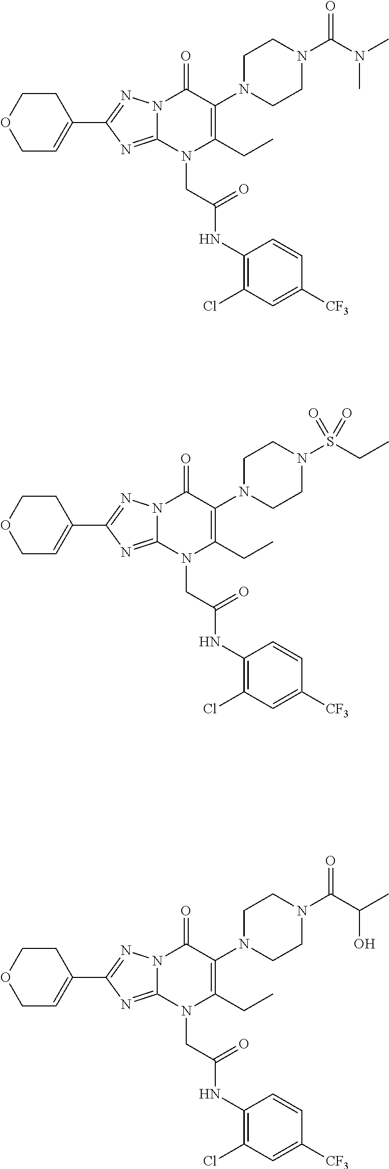 [SMILES: CCc1c(N2CCN(C(=O)N(C)C)CC2)c(=O)n2nc(C3=CCOCC3)nc2n1CC(=O)Nc1ccc(C(F)(F)F)cc1Cl ; CCc1c(N2CCN(S(=O)(=O)CC)CC2)c(=O)n2nc(C3=CCOCC3)nc2n1CC(=O)Nc1ccc(C(F)(F)F)cc1Cl ; CCc1c(N2CCN(C(=O)C(C)O)CC2)c(=O)n2nc(C3=CCOCC3)nc2n1CC(=O)Nc1ccc(C(F)(F)F)cc1Cl]

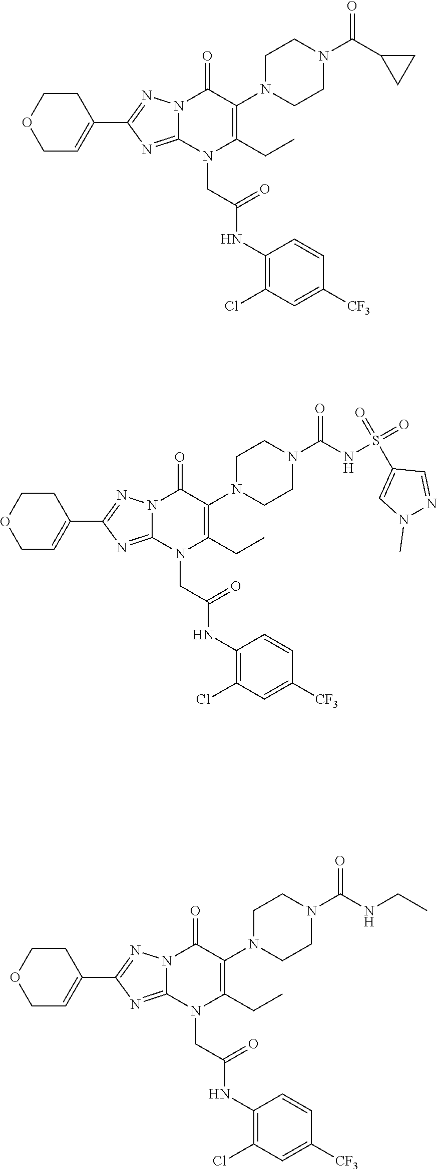 [SMILES: CCc1c(N2CCN(C(=O)C3CC3)CC2)c(=O)n2nc(C3=CCOCC3)nc2n1CC(=O)Nc1ccc(C(F)(F)F)cc1Cl ; CCc1c(N2CCN(C(=O)NS(=O)(=O)c3cnn(C)c3)CC2)c(=O)n2nc(C3=CCOCC3)nc2n1CC(=O)Nc1ccc(C(F)(F)F)cc1Cl ; CCNC(=O)N1CCN(c2c(CC)n(CC(=O)Nc3ccc(C(F)(F)F)cc3Cl)c3nc(C4=CCOCC4)nn3c2=O)CC1]

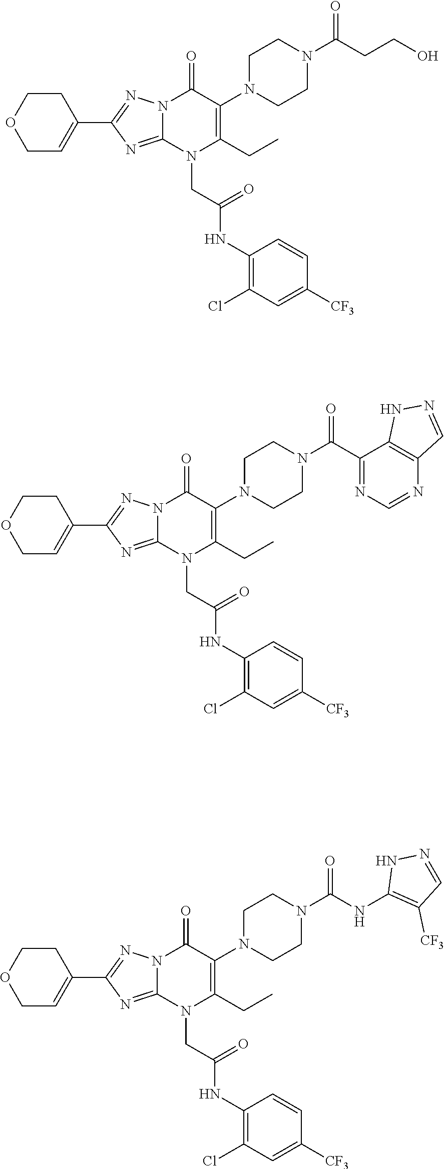 [SMILES: CCc1c(N2CCN(C(=O)CCO)CC2)c(=O)n2nc(C3=CCOCC3)nc2n1CC(=O)Nc1ccc(C(F)(F)F)cc1Cl ; CCc1c(N2CCN(C(=O)c3ncnc4cn[nH]c34)CC2)c(=O)n2nc(C3=CCOCC3)nc2n1CC(=O)Nc1ccc(C(F)(F)F)cc1Cl ; CCc1c(N2CCN(C(=O)Nc3[nH]ncc3C(F)(F)F)CC2)c(=O)n2nc(C3=CCOCC3)nc2n1CC(=O)Nc1ccc(C(F)(F)F)cc1Cl]

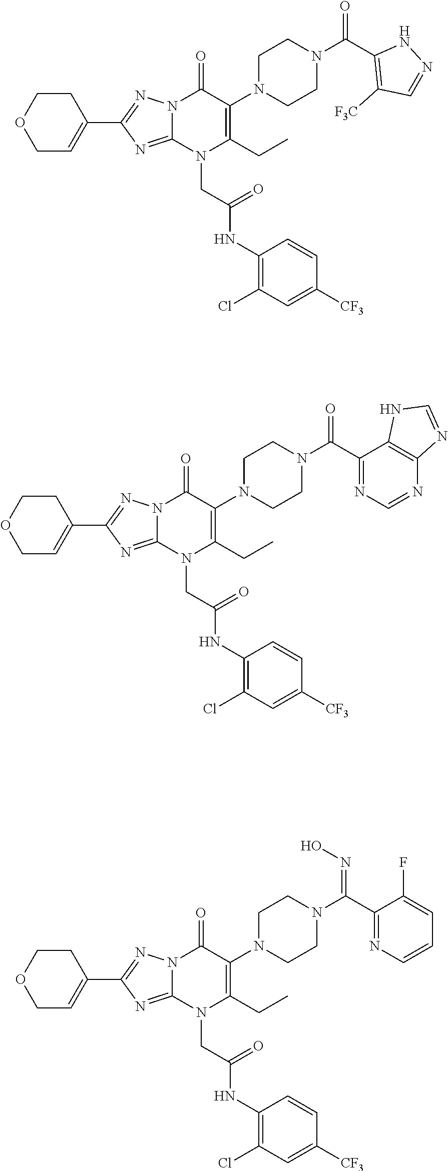 [SMILES: CCc1c(N2CCN(C(=O)c3[nH]ncc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3ncnc4nc[nH]c34)CC2)c(=O)n2nc(C3=CCOCC3)nc2n1CC(=O)Nc1ccc(C(F)(F)F)cc1Cl ; CCc1c(N2CCN(/C(=N\O)c3ncccc3F)CC2)c(=O)n2nc(C3=CCOCC3)nc2n1CC(=O)Nc1ccc(C(F)(F)F)cc1Cl]

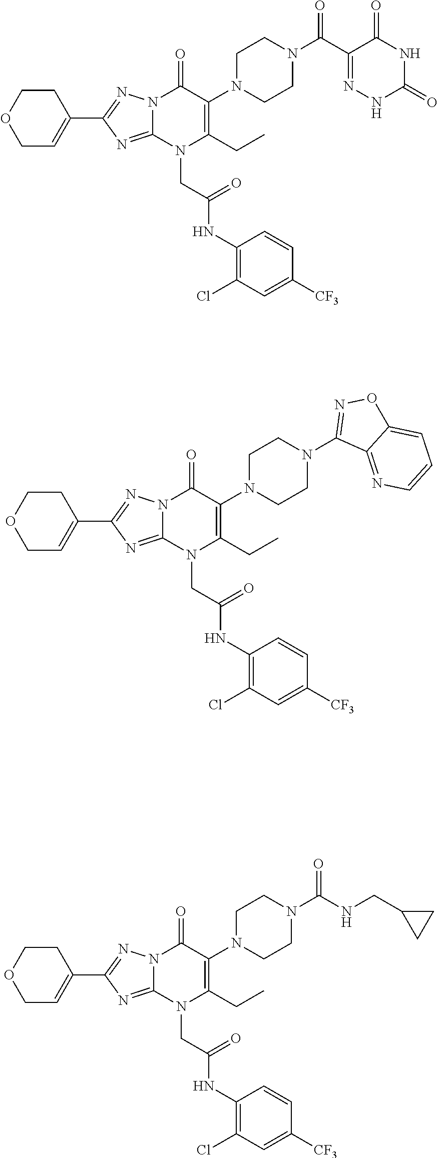 [SMILES: CCc1c(N2CCN(C(=O)c3n[nH]c(=O)[nH]c3=O)CC2)c(=O)n2nc(C3=CCOCC3)nc2n1CC(=O)Nc1ccc(C(F)(F)F)cc1Cl ; CCc1c(N2CCN(c3noc4cccnc34)CC2)c(=O)n2nc(C3=CCOCC3)nc2n1CC(=O)Nc1ccc(C(F)(F)F)cc1Cl ; CCc1c(N2CCN(C(=O)NCC3CC3)CC2)c(=O)n2nc(C3=CCOCC3)nc2n1CC(=O)Nc1ccc(C(F)(F)F)cc1Cl]

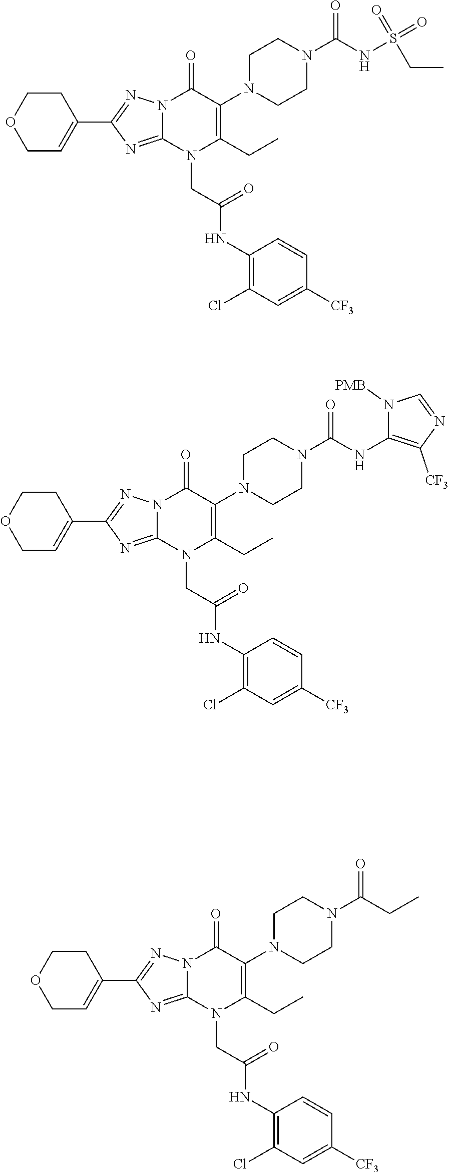

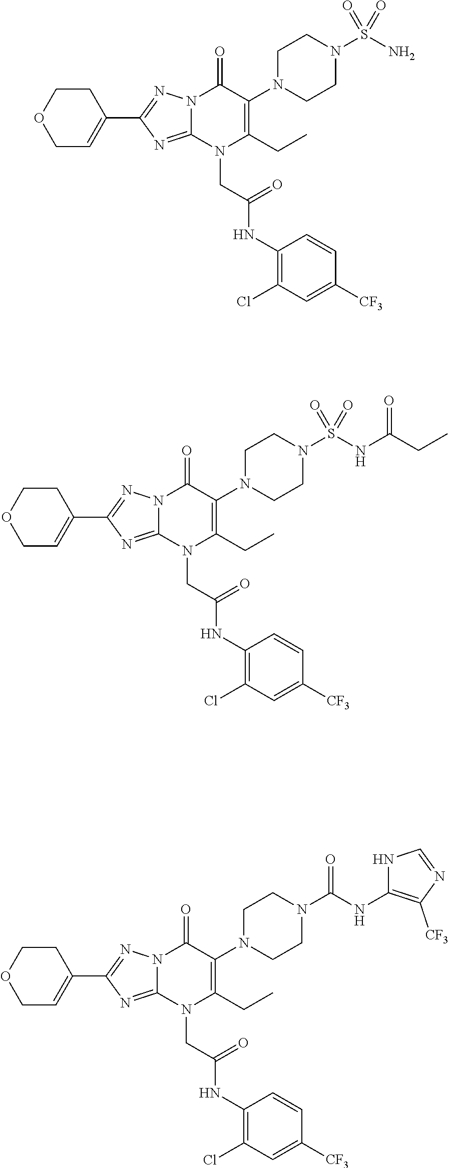 [SMILES: CCc1c(N2CCN(S(N)(=O)=O)CC2)c(=O)n2nc(C3=CCOCC3)nc2n1CC(=O)Nc1ccc(C(F)(F)F)cc1Cl ; CCC(=O)NS(=O)(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)Nc3[nH]cnc3C(F)(F)F)CC2)c(=O)n2nc(C3=CCOCC3)nc2n1CC(=O)Nc1ccc(C(F)(F)F)cc1Cl]

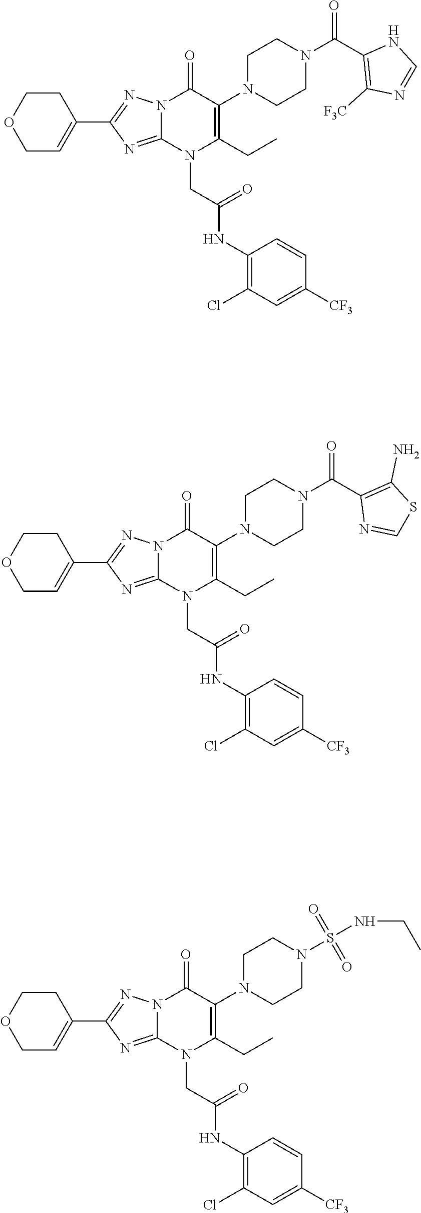 [SMILES: CCc1c(N2CCN(C(=O)c3[nH]cnc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3ncsc3N)CC2)c(=O)n2nc(C3=CCOCC3)nc2n1CC(=O)Nc1ccc(C(F)(F)F)cc1Cl ; CCNS(=O)(=O)N1CCN(c2c(CC)n(CC(=O)Nc3ccc(C(F)(F)F)cc3Cl)c3nc(C4=CCOCC4)nn3c2=O)CC1]

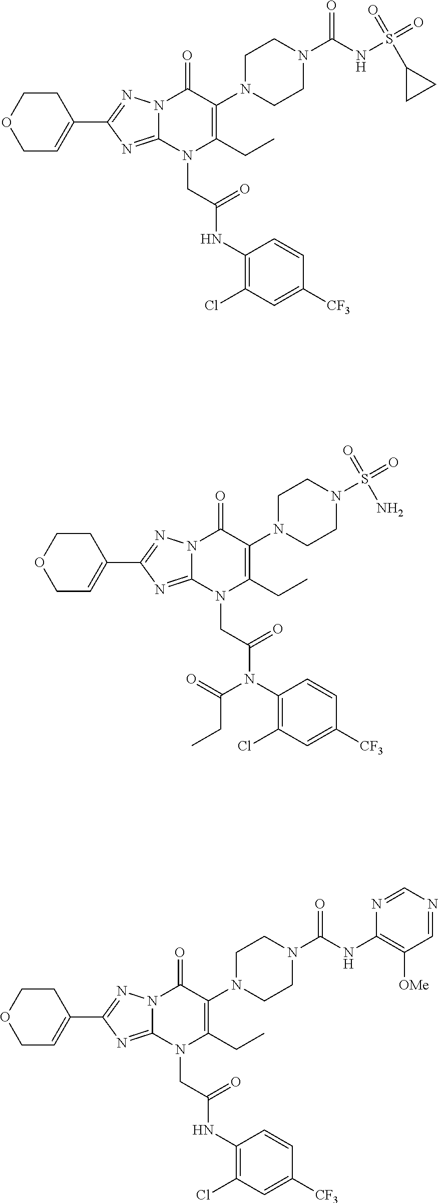 [SMILES: CCc1c(N2CCN(C(=O)NS(=O)(=O)C3CC3)CC2)c(=O)n2nc(C3=CCOCC3)nc2n1CC(=O)Nc1ccc(C(F)(F)F)cc1Cl ; CCC(=O)N(C(=O)Cn1c(CC)c(N2CCN(S(N)(=O)=O)CC2)c(=O)n2nc(C3=CCOCC3)nc12)c1ccc(C(F)(F)F)cc1Cl ; CCc1c(N2CCN(C(=O)Nc3ncncc3OC)CC2)c(=O)n2nc(C3=CCOCC3)nc2n1CC(=O)Nc1ccc(C(F)(F)F)cc1Cl]

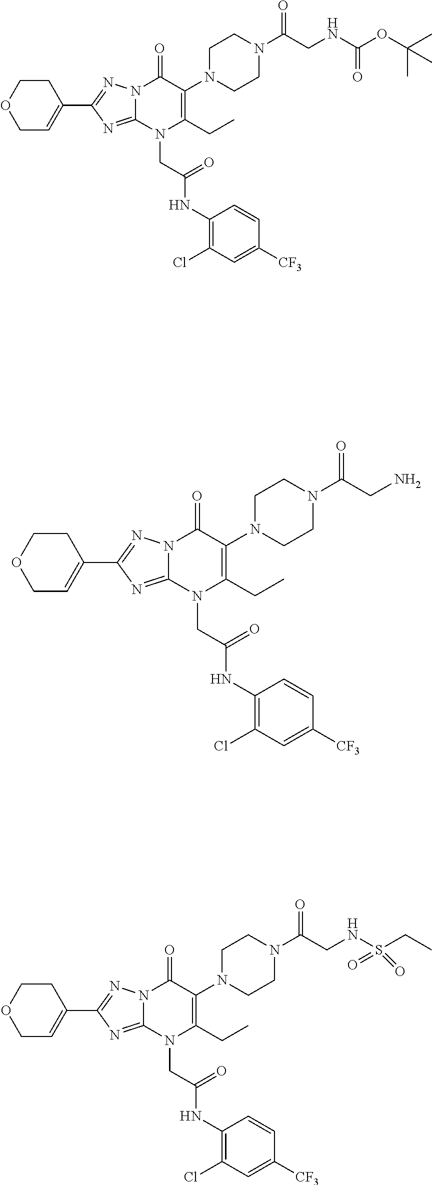 [SMILES: CCc1c(N2CCN(C(=O)CNC(=O)OC(C)(C)C)CC2)c(=O)n2nc(C3=CCOCC3)nc2n1CC(=O)Nc1ccc(C(F)(F)F)cc1Cl ; CCc1c(N2CCN(C(=O)CN)CC2)c(=O)n2nc(C3=CCOCC3)nc2n1CC(=O)Nc1ccc(C(F)(F)F)cc1Cl ; CCc1c(N2CCN(C(=O)CNS(=O)(=O)CC)CC2)c(=O)n2nc(C3=CCOCC3)nc2n1CC(=O)Nc1ccc(C(F)(F)F)cc1Cl]

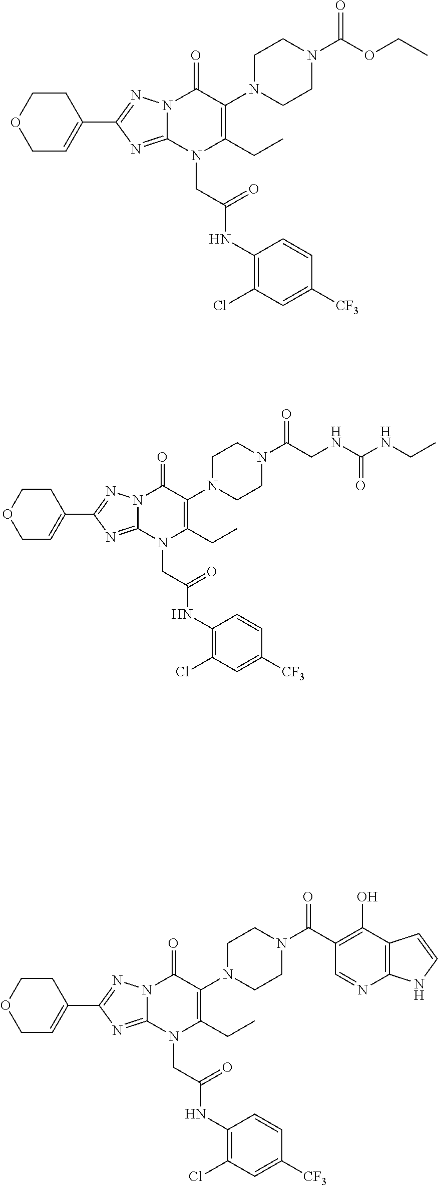 [SMILES: CCOC(=O)N1CCN(c2c(CC)n(CC(=O)Nc3ccc(C(F)(F)F)cc3Cl)c3nc(C4=CCOCC4)nn3c2=O)CC1 ; CCNC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)c3cnc4[nH]ccc4c3O)CC2)c(=O)n2nc(C3=CCOCC3)nc2n1CC(=O)Nc1ccc(C(F)(F)F)cc1Cl]

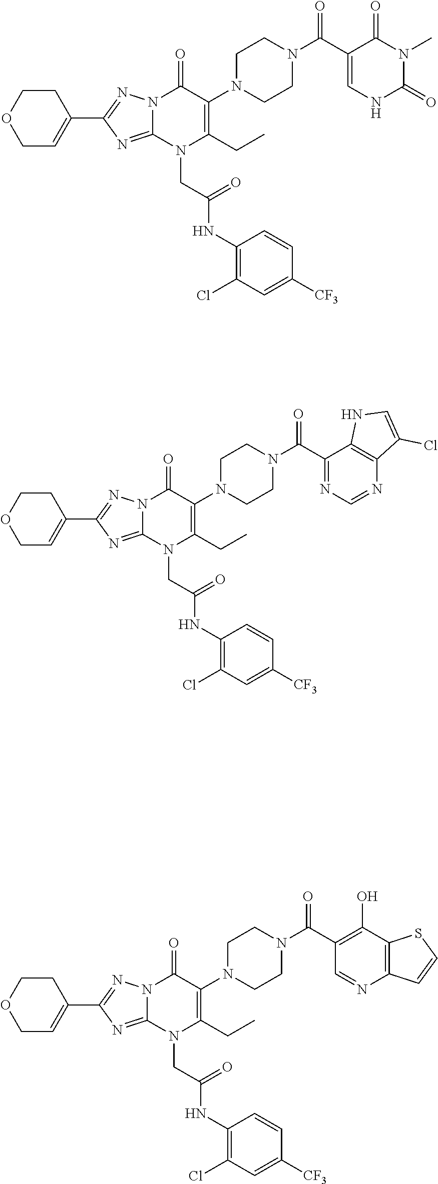 [SMILES: CCc1c(N2CCN(C(=O)c3c[nH]c(=O)n(C)c3=O)CC2)c(=O)n2nc(C3=CCOCC3)nc2n1CC(=O)Nc1ccc(C(F)(F)F)cc1Cl ; CCc1c(N2CCN(C(=O)c3ncnc4c(Cl)c[nH]c34)CC2)c(=O)n2nc(C3=CCOCC3)nc2n1CC(=O)Nc1ccc(C(F)(F)F)cc1Cl ; CCc1c(N2CCN(C(=O)c3cnc4ccsc4c3O)CC2)c(=O)n2nc(C3=CCOCC3)nc2n1CC(=O)Nc1ccc(C(F)(F)F)cc1Cl]

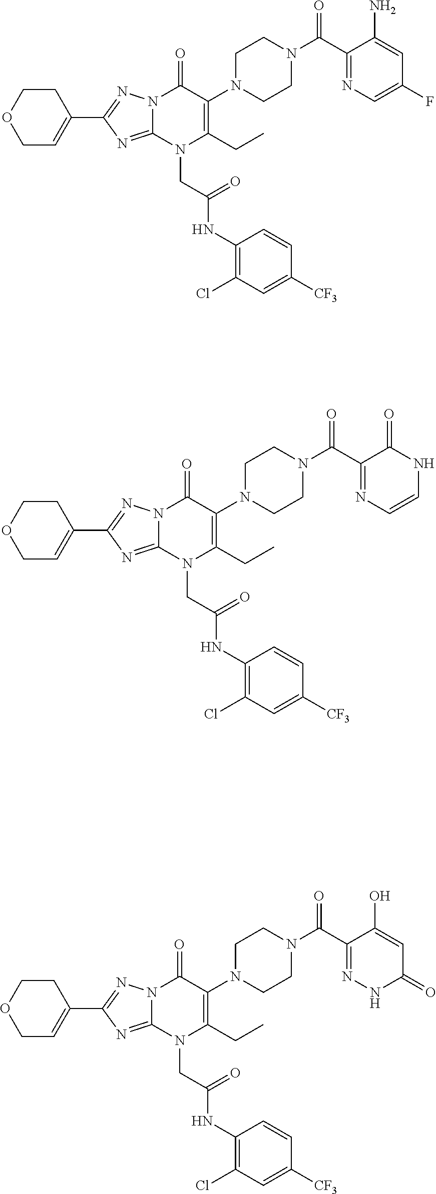 [SMILES: CCc1c(N2CCN(C(=O)c3ncc(F)cc3N)CC2)c(=O)n2nc(C3=CCOCC3)nc2n1CC(=O)Nc1ccc(C(F)(F)F)cc1Cl ; CCc1c(N2CCN(C(=O)c3ncc[nH]c3=O)CC2)c(=O)n2nc(C3=CCOCC3)nc2n1CC(=O)Nc1ccc(C(F)(F)F)cc1Cl ; CCc1c(N2CCN(C(=O)c3n[nH]c(=O)cc3O)CC2)c(=O)n2nc(C3=CCOCC3)nc2n1CC(=O)Nc1ccc(C(F)(F)F)cc1Cl]

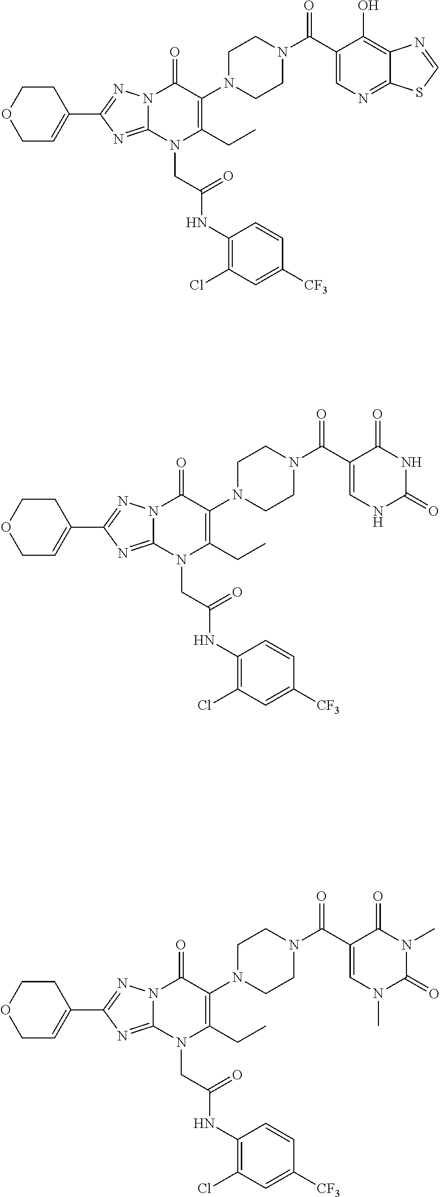 [SMILES: CCc1c(N2CCN(C(=O)c3cnc4scnc4c3O)CC2)c(=O)n2nc(C3=CCOCC3)nc2n1CC(=O)Nc1ccc(C(F)(F)F)cc1Cl ; CCc1c(N2CCN(C(=O)c3c[nH]c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cn(C)c(=O)n(C)c3=O)CC2)c(=O)n2nc(C3=CCOCC3)nc2n1CC(=O)Nc1ccc(C(F)(F)F)cc1Cl]

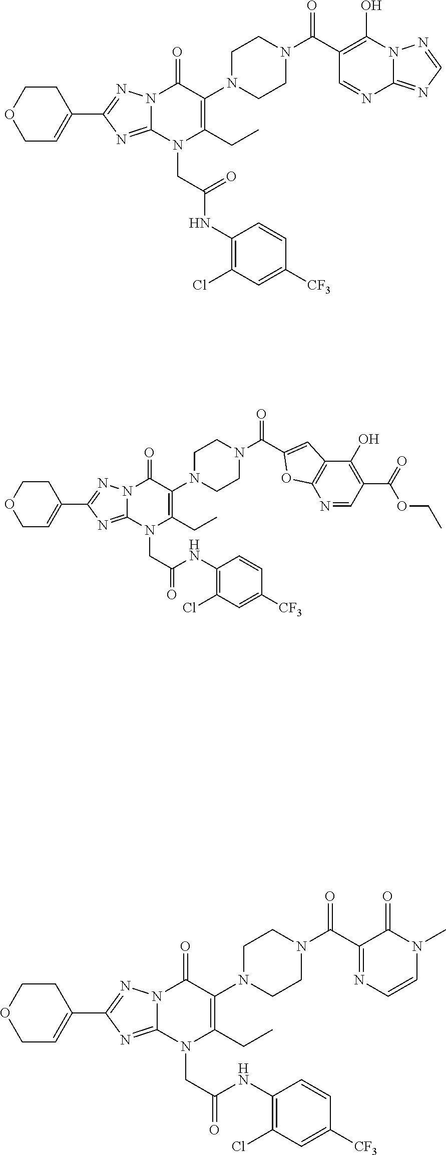 [SMILES: CCc1c(N2CCN(C(=O)c3cnc4ncnn4c3O)CC2)c(=O)n2nc(C3=CCOCC3)nc2n1CC(=O)Nc1ccc(C(F)(F)F)cc1Cl ; CCOC(=O)c1cnc2oc(C(=O)N3CCN(c4c(CC)n(CC(=O)Nc5ccc(C(F)(F)F)cc5Cl)c5nc(C6=CCOCC6)nn5c4=O)CC3)cc2c1O ; CCc1c(N2CCN(C(=O)c3nccn(C)c3=O)CC2)c(=O)n2nc(C3=CCOCC3)nc2n1CC(=O)Nc1ccc(C(F)(F)F)cc1Cl]

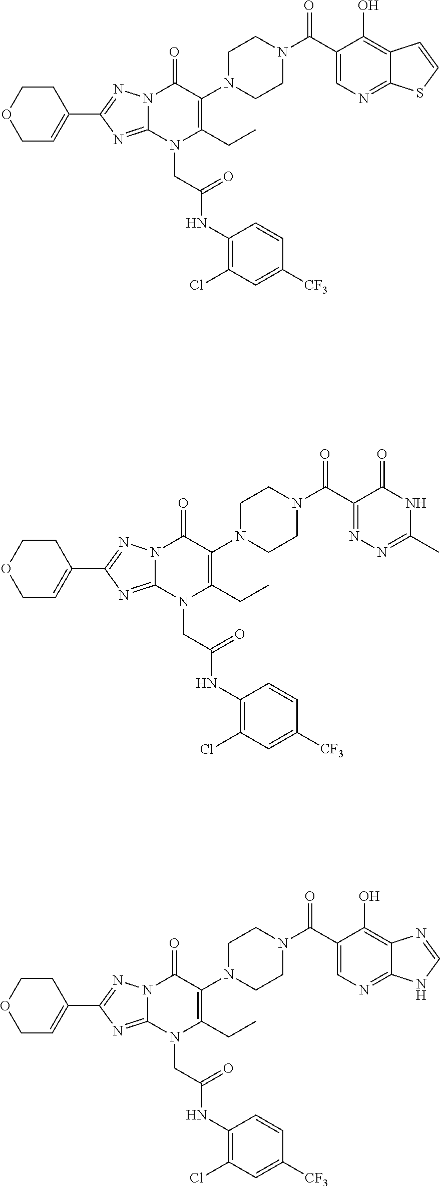 [SMILES: CCc1c(N2CCN(C(=O)c3cnc4sccc4c3O)CC2)c(=O)n2nc(C3=CCOCC3)nc2n1CC(=O)Nc1ccc(C(F)(F)F)cc1Cl ; CCc1c(N2CCN(C(=O)c3nnc(C)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cnc4[nH]cnc4c3O)CC2)c(=O)n2nc(C3=CCOCC3)nc2n1CC(=O)Nc1ccc(C(F)(F)F)cc1Cl]

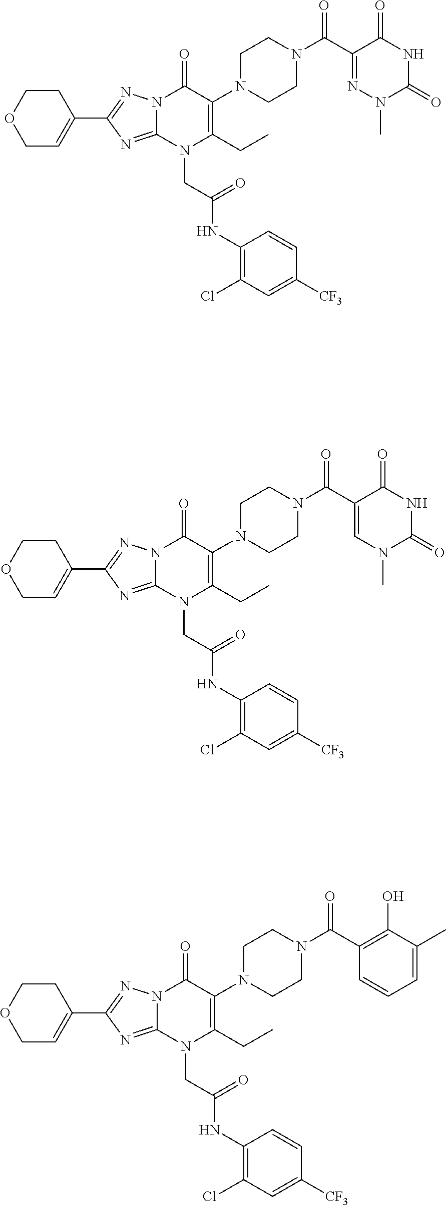 [SMILES: CCc1c(N2CCN(C(=O)c3nn(C)c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cn(C)c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cccc(C)c3O)CC2)c(=O)n2nc(C3=CCOCC3)nc2n1CC(=O)Nc1ccc(C(F)(F)F)cc1Cl]

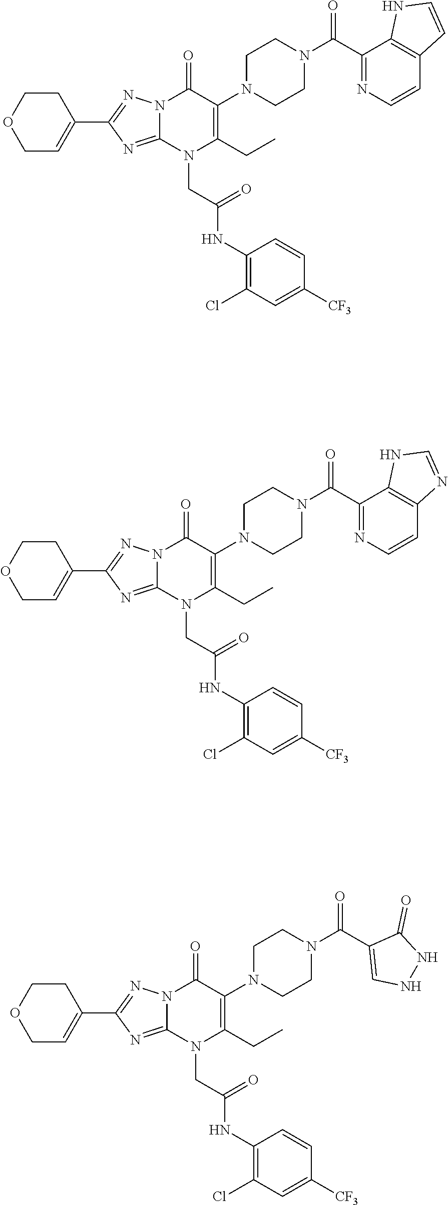 [SMILES: CCc1c(N2CCN(C(=O)c3nccc4cc[nH]c34)CC2)c(=O)n2nc(C3=CCOCC3)nc2n1CC(=O)Nc1ccc(C(F)(F)F)cc1Cl ; CCc1c(N2CCN(C(=O)c3nccc4nc[nH]c34)CC2)c(=O)n2nc(C3=CCOCC3)nc2n1CC(=O)Nc1ccc(C(F)(F)F)cc1Cl ; CCc1c(N2CCN(C(=O)c3c[nH][nH]c3=O)CC2)c(=O)n2nc(C3=CCOCC3)nc2n1CC(=O)Nc1ccc(C(F)(F)F)cc1Cl]

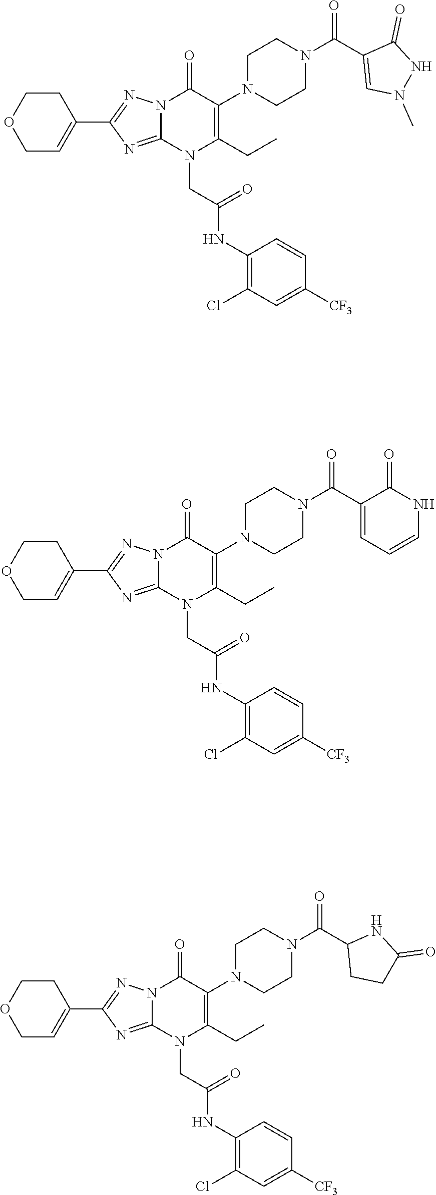 [SMILES: CCc1c(N2CCN(C(=O)c3cn(C)[nH]c3=O)CC2)c(=O)n2nc(C3=CCOCC3)nc2n1CC(=O)Nc1ccc(C(F)(F)F)cc1Cl ; CCc1c(N2CCN(C(=O)c3ccc[nH]c3=O)CC2)c(=O)n2nc(C3=CCOCC3)nc2n1CC(=O)Nc1ccc(C(F)(F)F)cc1Cl ; CCc1c(N2CCN(C(=O)C3CCC(=O)N3)CC2)c(=O)n2nc(C3=CCOCC3)nc2n1CC(=O)Nc1ccc(C(F)(F)F)cc1Cl]

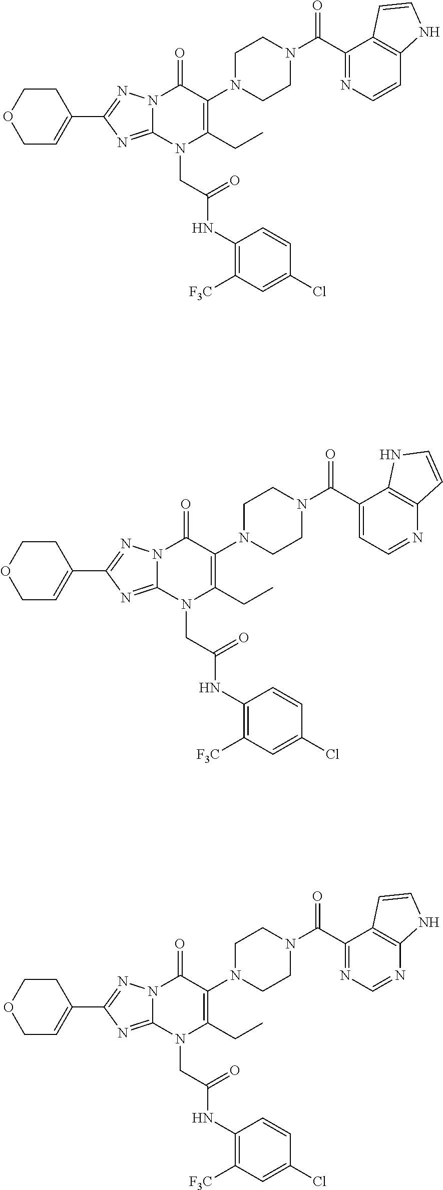 [SMILES: CCc1c(N2CCN(C(=O)c3nccc4[nH]ccc34)CC2)c(=O)n2nc(C3=CCOCC3)nc2n1CC(=O)Nc1ccc(Cl)cc1C(F)(F)F ; CCc1c(N2CCN(C(=O)c3ccnc4cc[nH]c34)CC2)c(=O)n2nc(C3=CCOCC3)nc2n1CC(=O)Nc1ccc(Cl)cc1C(F)(F)F ; CCc1c(N2CCN(C(=O)c3ncnc4[nH]ccc34)CC2)c(=O)n2nc(C3=CCOCC3)nc2n1CC(=O)Nc1ccc(Cl)cc1C(F)(F)F]

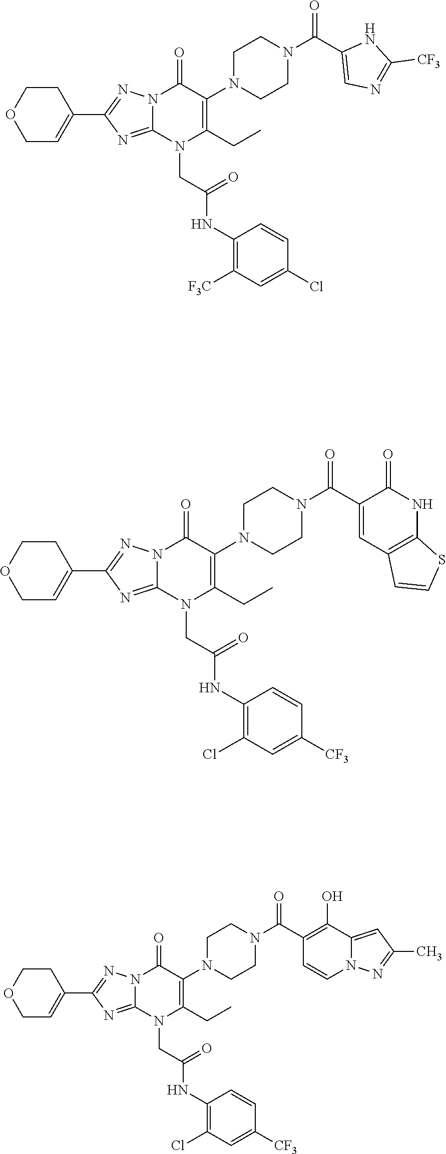 [SMILES: CCc1c(N2CCN(C(=O)c3cnc(C(F)(F)F)[nH]3)CC2)c(=O)n2nc(C3=CCOCC3)nc2n1CC(=O)Nc1ccc(Cl)cc1C(F)(F)F ; CCc1c(N2CCN(C(=O)c3cc4ccsc4[nH]c3=O)CC2)c(=O)n2nc(C3=CCOCC3)nc2n1CC(=O)Nc1ccc(C(F)(F)F)cc1Cl ; CCc1c(N2CCN(C(=O)c3ccn4nc(C)cc4c3O)CC2)c(=O)n2nc(C3=CCOCC3)nc2n1CC(=O)Nc1ccc(C(F)(F)F)cc1Cl]

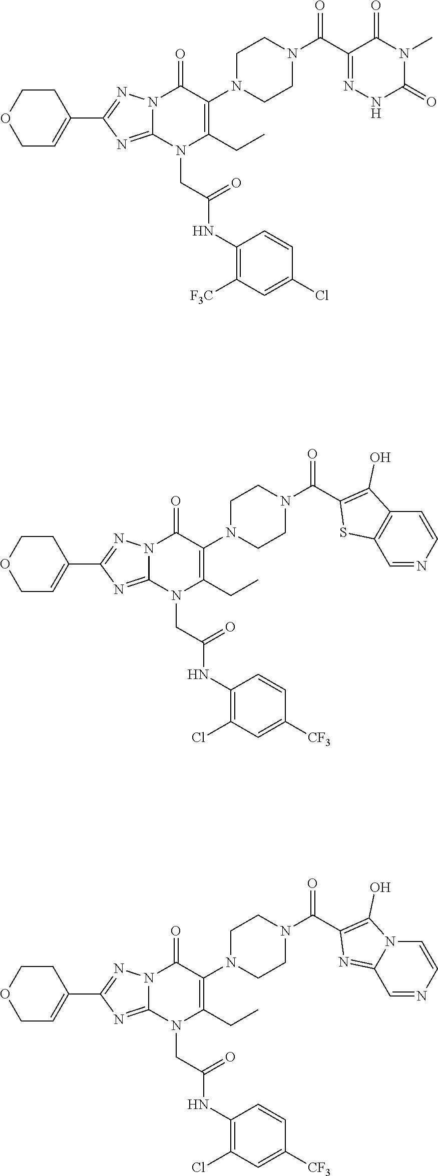 [SMILES: CCc1c(N2CCN(C(=O)c3n[nH]c(=O)n(C)c3=O)CC2)c(=O)n2nc(C3=CCOCC3)nc2n1CC(=O)Nc1ccc(Cl)cc1C(F)(F)F ; CCc1c(N2CCN(C(=O)c3sc4cnccc4c3O)CC2)c(=O)n2nc(C3=CCOCC3)nc2n1CC(=O)Nc1ccc(C(F)(F)F)cc1Cl ; CCc1c(N2CCN(C(=O)c3nc4cnccn4c3O)CC2)c(=O)n2nc(C3=CCOCC3)nc2n1CC(=O)Nc1ccc(C(F)(F)F)cc1Cl]

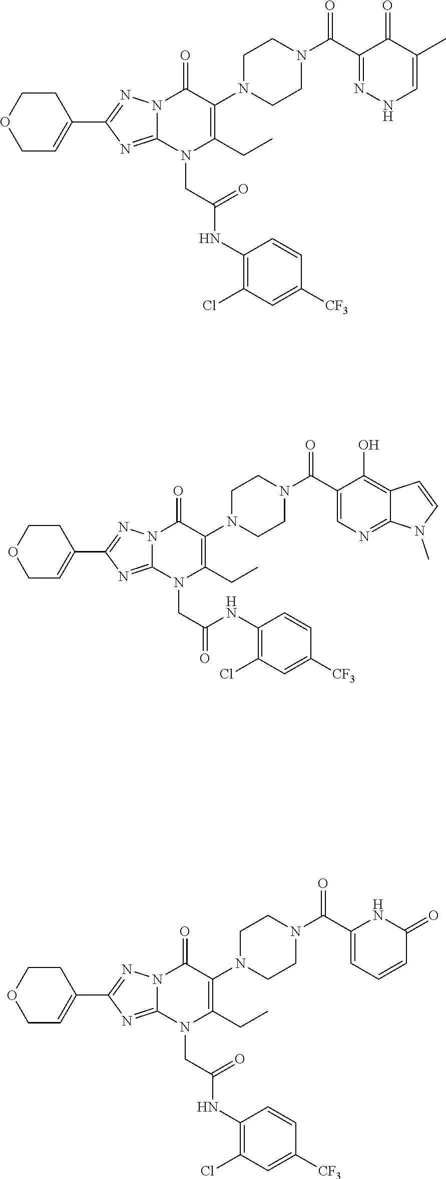 [SMILES: CCc1c(N2CCN(C(=O)c3n[nH]cc(C)c3=O)CC2)c(=O)n2nc(C3=CCOCC3)nc2n1CC(=O)Nc1ccc(C(F)(F)F)cc1Cl ; CCc1c(N2CCN(C(=O)c3cnc4c(ccn4C)c3O)CC2)c(=O)n2nc(C3=CCOCC3)nc2n1CC(=O)Nc1ccc(C(F)(F)F)cc1Cl ; CCc1c(N2CCN(C(=O)c3cccc(=O)[nH]3)CC2)c(=O)n2nc(C3=CCOCC3)nc2n1CC(=O)Nc1ccc(C(F)(F)F)cc1Cl]

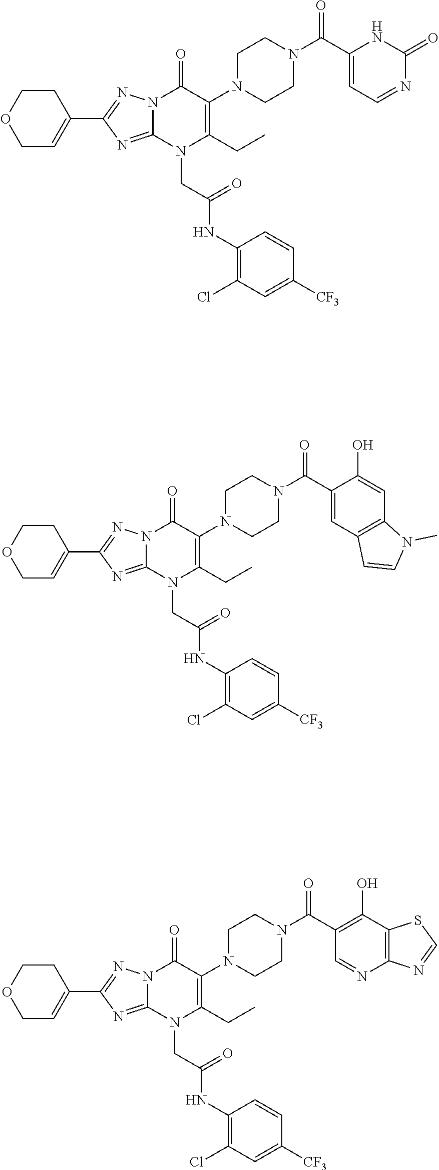 [SMILES: CCc1c(N2CCN(C(=O)c3ccnc(=O)[nH]3)CC2)c(=O)n2nc(C3=CCOCC3)nc2n1CC(=O)Nc1ccc(C(F)(F)F)cc1Cl ; CCc1c(N2CCN(C(=O)c3cc4ccn(C)c4cc3O)CC2)c(=O)n2nc(C3=CCOCC3)nc2n1CC(=O)Nc1ccc(C(F)(F)F)cc1Cl ; CCc1c(N2CCN(C(=O)c3cnc4ncsc4c3O)CC2)c(=O)n2nc(C3=CCOCC3)nc2n1CC(=O)Nc1ccc(C(F)(F)F)cc1Cl]

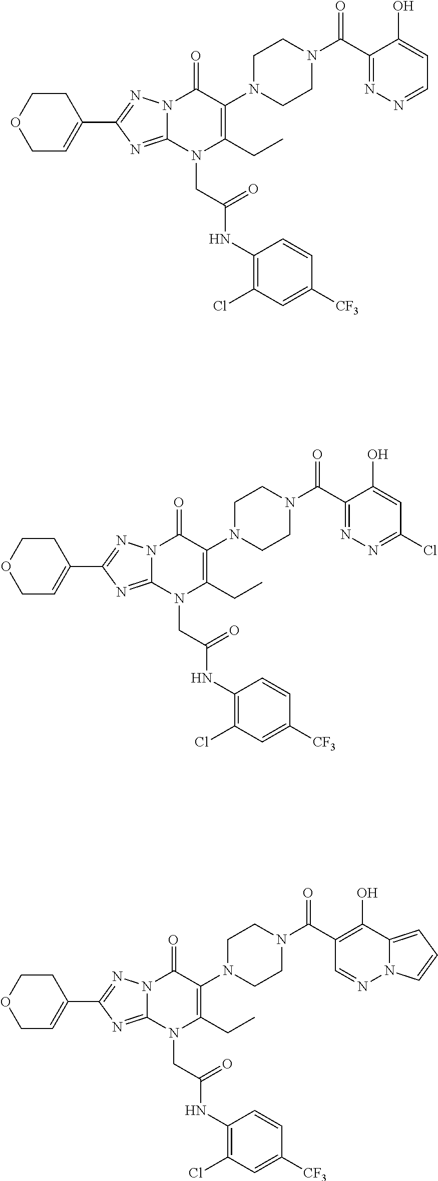 [SMILES: CCc1c(N2CCN(C(=O)c3nnccc3O)CC2)c(=O)n2nc(C3=CCOCC3)nc2n1CC(=O)Nc1ccc(C(F)(F)F)cc1Cl ; CCc1c(N2CCN(C(=O)c3nnc(Cl)cc3O)CC2)c(=O)n2nc(C3=CCOCC3)nc2n1CC(=O)Nc1ccc(C(F)(F)F)cc1Cl ; CCc1c(N2CCN(C(=O)c3cnn4cccc4c3O)CC2)c(=O)n2nc(C3=CCOCC3)nc2n1CC(=O)Nc1ccc(C(F)(F)F)cc1Cl]

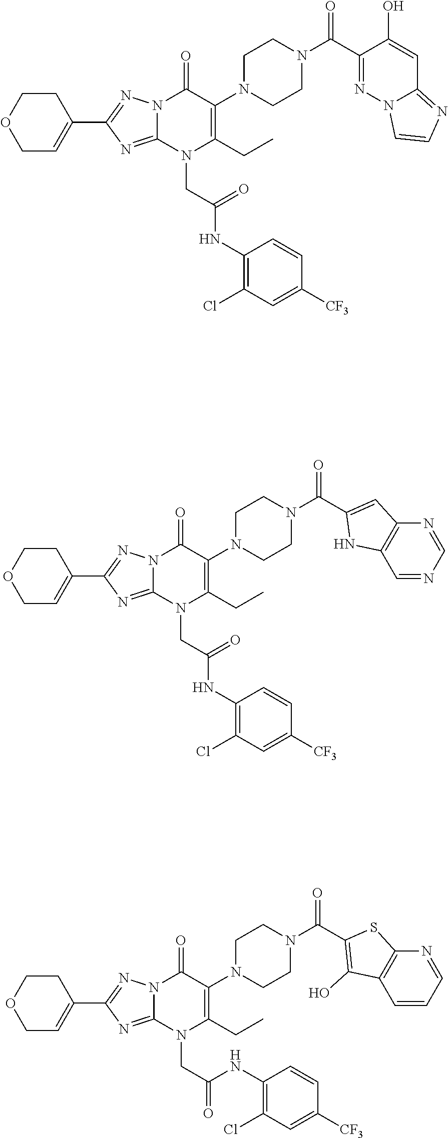 [SMILES: CCc1c(N2CCN(C(=O)c3nn4ccnc4cc3O)CC2)c(=O)n2nc(C3=CCOCC3)nc2n1CC(=O)Nc1ccc(C(F)(F)F)cc1Cl ; CCc1c(N2CCN(C(=O)c3cc4ncncc4[nH]3)CC2)c(=O)n2nc(C3=CCOCC3)nc2n1CC(=O)Nc1ccc(C(F)(F)F)cc1Cl ; CCc1c(N2CCN(C(=O)c3sc4ncccc4c3O)CC2)c(=O)n2nc(C3=CCOCC3)nc2n1CC(=O)Nc1ccc(C(F)(F)F)cc1Cl]

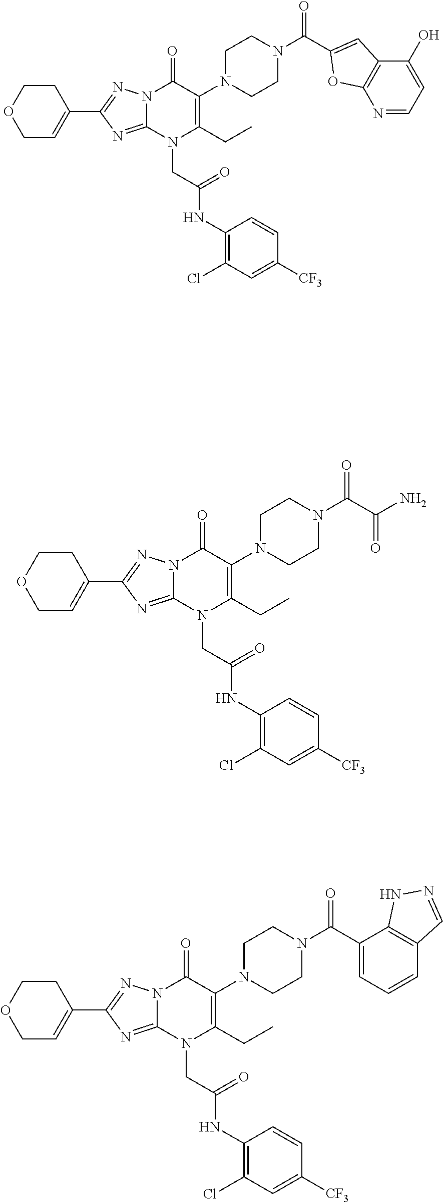 [SMILES: CCc1c(N2CCN(C(=O)c3cc4c(O)ccnc4o3)CC2)c(=O)n2nc(C3=CCOCC3)nc2n1CC(=O)Nc1ccc(C(F)(F)F)cc1Cl ; CCc1c(N2CCN(C(=O)C(N)=O)CC2)c(=O)n2nc(C3=CCOCC3)nc2n1CC(=O)Nc1ccc(C(F)(F)F)cc1Cl ; CCc1c(N2CCN(C(=O)c3cccc4cn[nH]c34)CC2)c(=O)n2nc(C3=CCOCC3)nc2n1CC(=O)Nc1ccc(C(F)(F)F)cc1Cl]

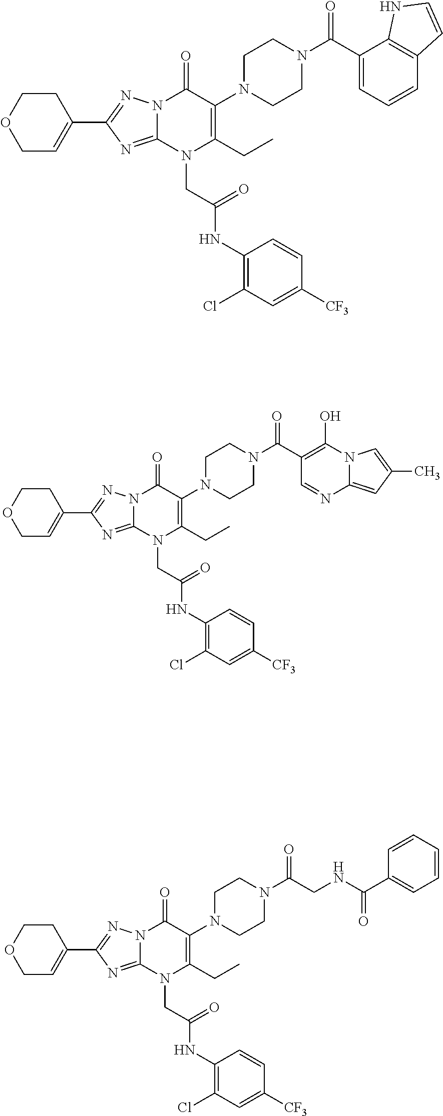 [SMILES: CCc1c(N2CCN(C(=O)c3cccc4cc[nH]c34)CC2)c(=O)n2nc(C3=CCOCC3)nc2n1CC(=O)Nc1ccc(C(F)(F)F)cc1Cl ; CCc1c(N2CCN(C(=O)c3cnc4cc(C)cn4c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3)CC2)c(=O)n2nc(C3=CCOCC3)nc2n1CC(=O)Nc1ccc(C(F)(F)F)cc1Cl]

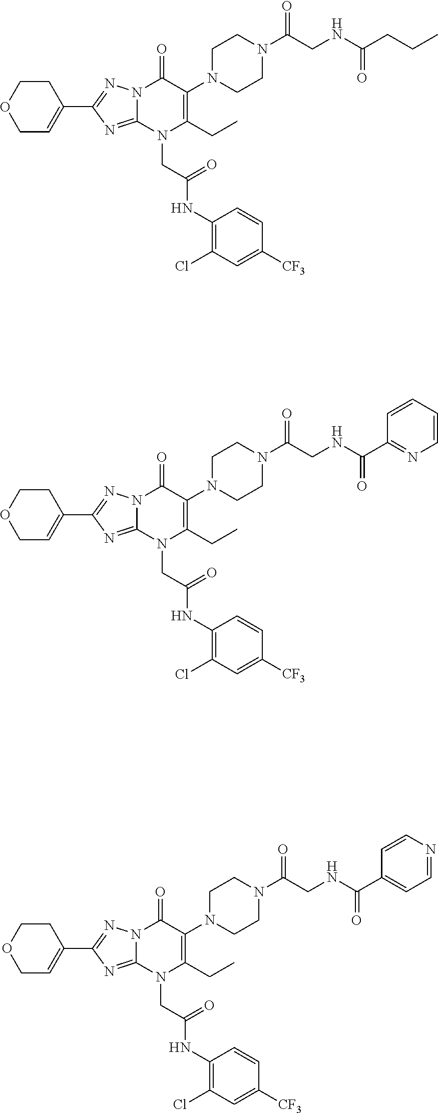 [SMILES: CCCC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)CNC(=O)c3ccccn3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccncc3)CC2)c(=O)n2nc(C3=CCOCC3)nc2n1CC(=O)Nc1ccc(C(F)(F)F)cc1Cl]

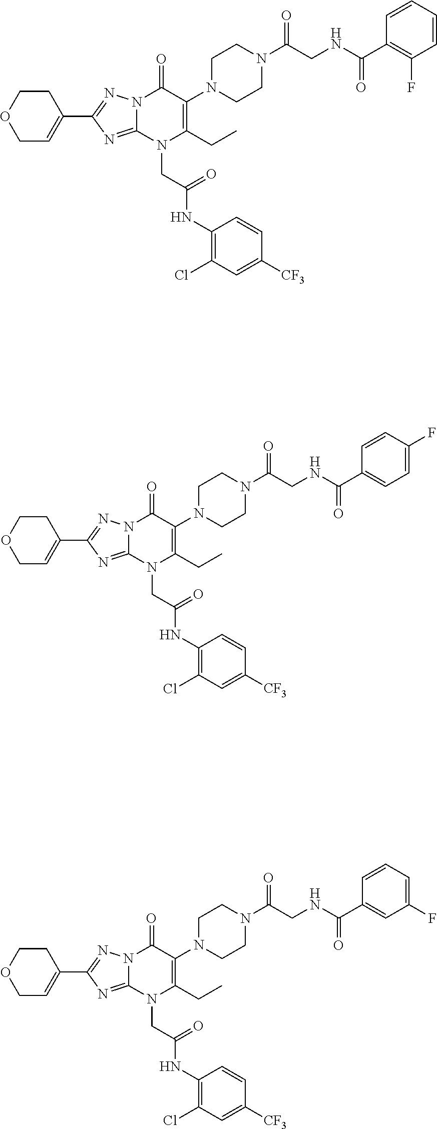 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)cc3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(F)c3)CC2)c(=O)n2nc(C3=CCOCC3)nc2n1CC(=O)Nc1ccc(C(F)(F)F)cc1Cl]

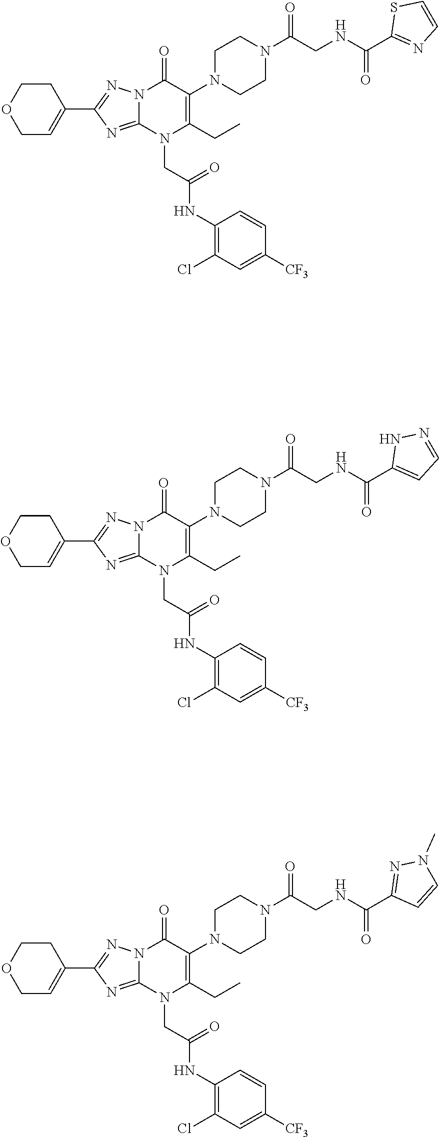 [SMILES: CCc1c(N2CCN(C(=O)CNC(=O)c3nccs3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccn[nH]3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccn(C)n3)CC2)c(=O)n2nc(C3=CCOCC3)nc2n1CC(=O)Nc1ccc(C(F)(F)F)cc1Cl]

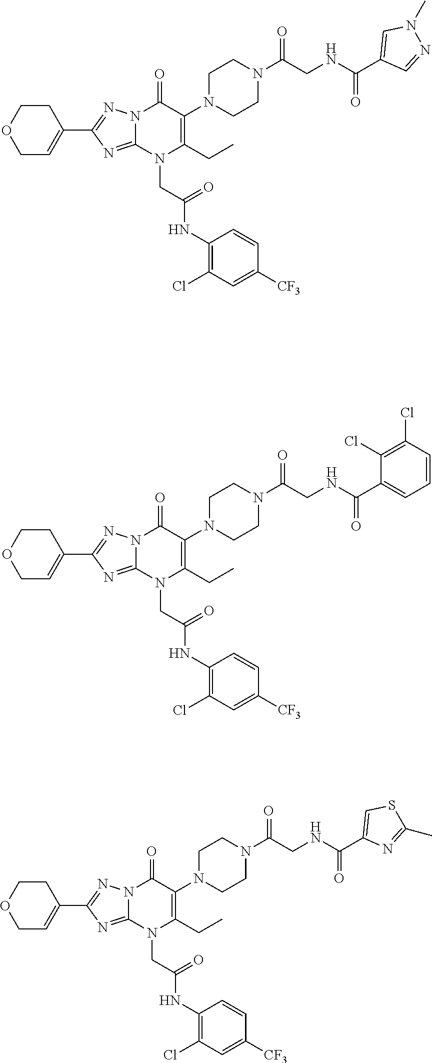 [SMILES: CCc1c(N2CCN(C(=O)CNC(=O)c3cnn(C)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(Cl)c3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3csc(C)n3)CC2)c(=O)n2nc(C3=CCOCC3)nc2n1CC(=O)Nc1ccc(C(F)(F)F)cc1Cl]

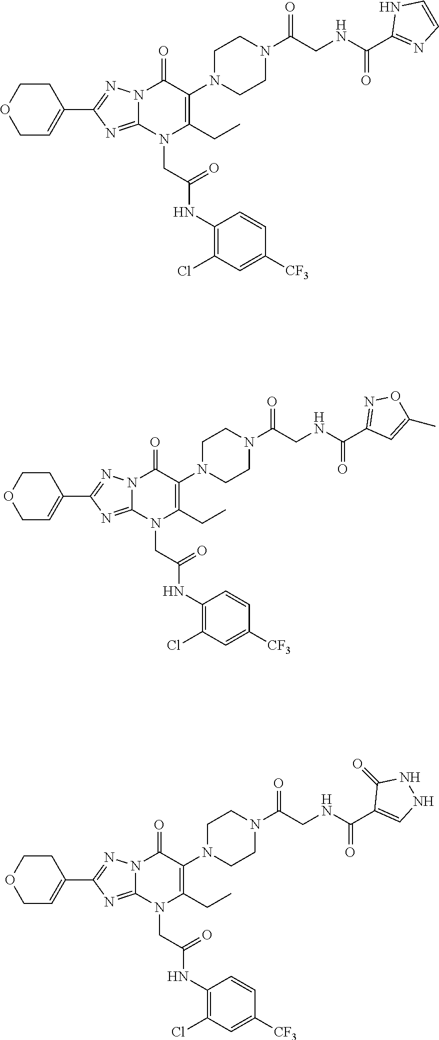 [SMILES: CCc1c(N2CCN(C(=O)CNC(=O)c3ncc[nH]3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C)on3)CC2)c(=O)n2nc(C3=CCOCC3)nc2n1CC(=O)Nc1ccc(C(F)(F)F)cc1Cl ; CCc1c(N2CCN(C(=O)CNC(=O)c3c[nH][nH]c3=O)CC2)c(=O)n2nc(C3=CCOCC3)nc2n1CC(=O)Nc1ccc(C(F)(F)F)cc1Cl]

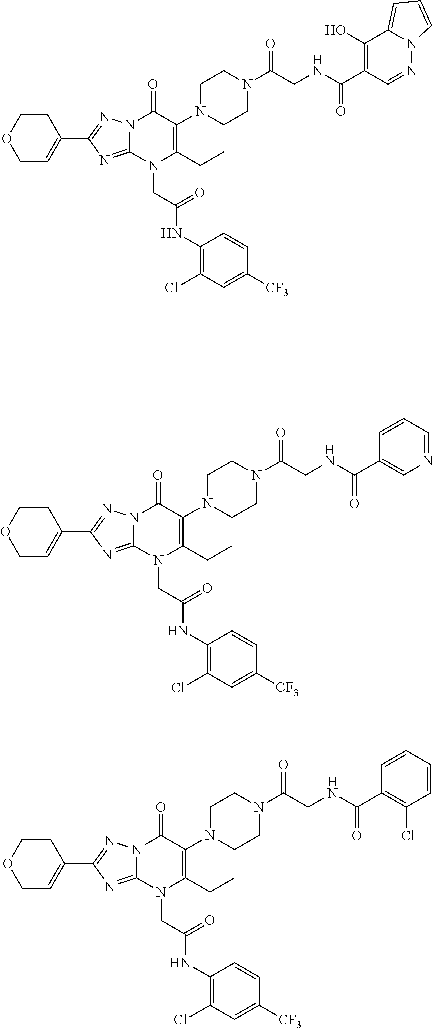 [SMILES: CCc1c(N2CCN(C(=O)CNC(=O)c3cnn4cccc4c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cccnc3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3Cl)CC2)c(=O)n2nc(C3=CCOCC3)nc2n1CC(=O)Nc1ccc(C(F)(F)F)cc1Cl]

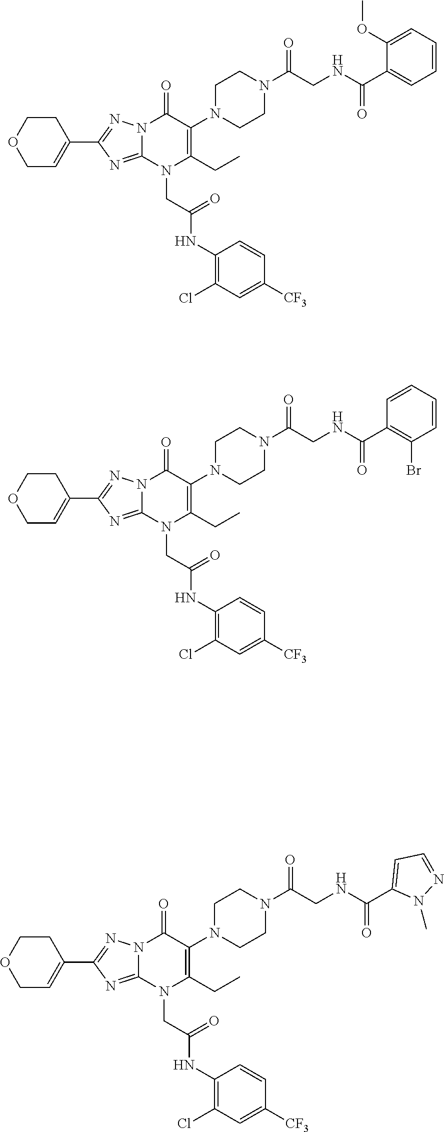 [SMILES: CCc1c(N2CCN(C(=O)CNC(=O)c3ccccc3OC)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3Br)CC2)c(=O)n2nc(C3=CCOCC3)nc2n1CC(=O)Nc1ccc(C(F)(F)F)cc1Cl ; CCc1c(N2CCN(C(=O)CNC(=O)c3ccnn3C)CC2)c(=O)n2nc(C3=CCOCC3)nc2n1CC(=O)Nc1ccc(C(F)(F)F)cc1Cl]

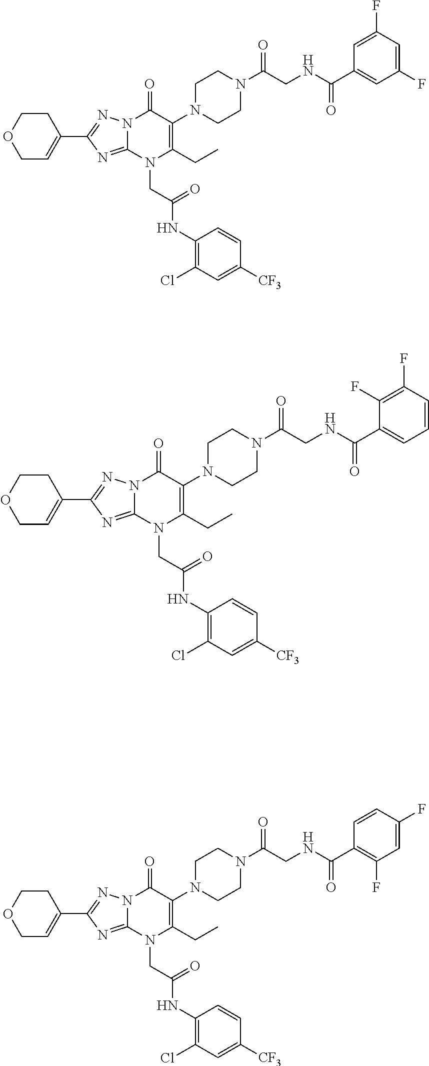 [SMILES: CCc1c(N2CCN(C(=O)CNC(=O)c3cc(F)cc(F)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(F)c3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)cc3F)CC2)c(=O)n2nc(C3=CCOCC3)nc2n1CC(=O)Nc1ccc(C(F)(F)F)cc1Cl]

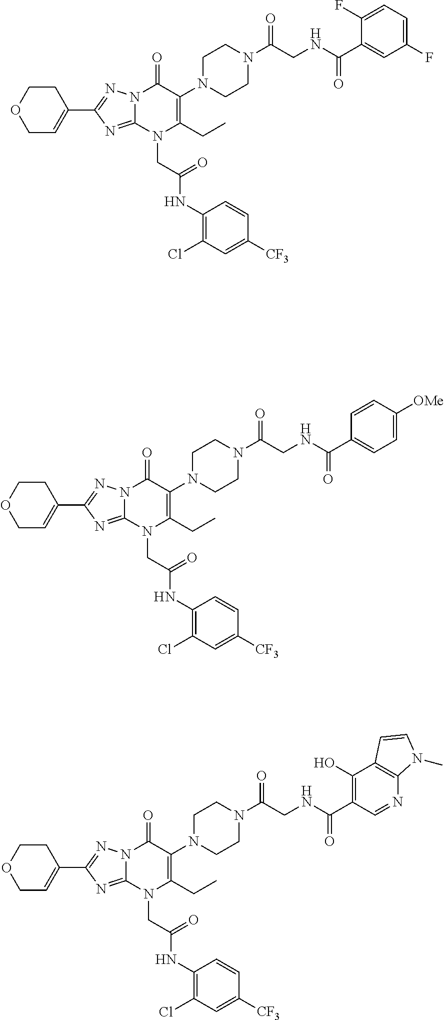 [SMILES: CCc1c(N2CCN(C(=O)CNC(=O)c3cc(F)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(OC)cc3)CC2)c(=O)n2nc(C3=CCOCC3)nc2n1CC(=O)Nc1ccc(C(F)(F)F)cc1Cl ; CCc1c(N2CCN(C(=O)CNC(=O)c3cnc4c(ccn4C)c3O)CC2)c(=O)n2nc(C3=CCOCC3)nc2n1CC(=O)Nc1ccc(C(F)(F)F)cc1Cl]

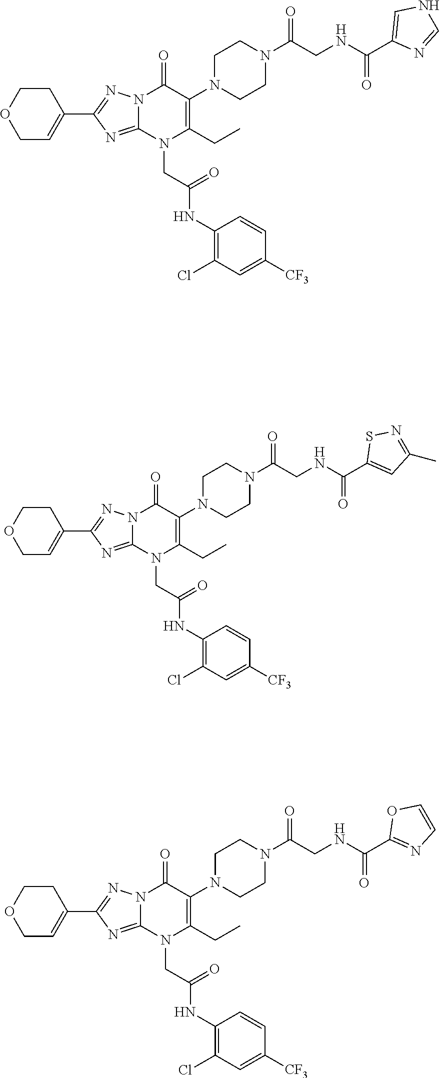 [SMILES: CCc1c(N2CCN(C(=O)CNC(=O)c3c[nH]cn3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C)ns3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncco3)CC2)c(=O)n2nc(C3=CCOCC3)nc2n1CC(=O)Nc1ccc(C(F)(F)F)cc1Cl]

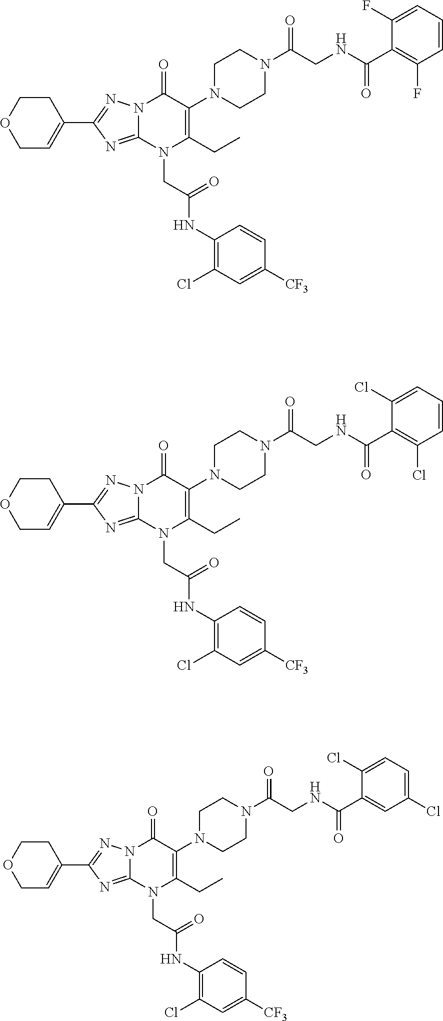 [SMILES: CCc1c(N2CCN(C(=O)CNC(=O)c3c(F)cccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3c(Cl)cccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(Cl)ccc3Cl)CC2)c(=O)n2nc(C3=CCOCC3)nc2n1CC(=O)Nc1ccc(C(F)(F)F)cc1Cl]

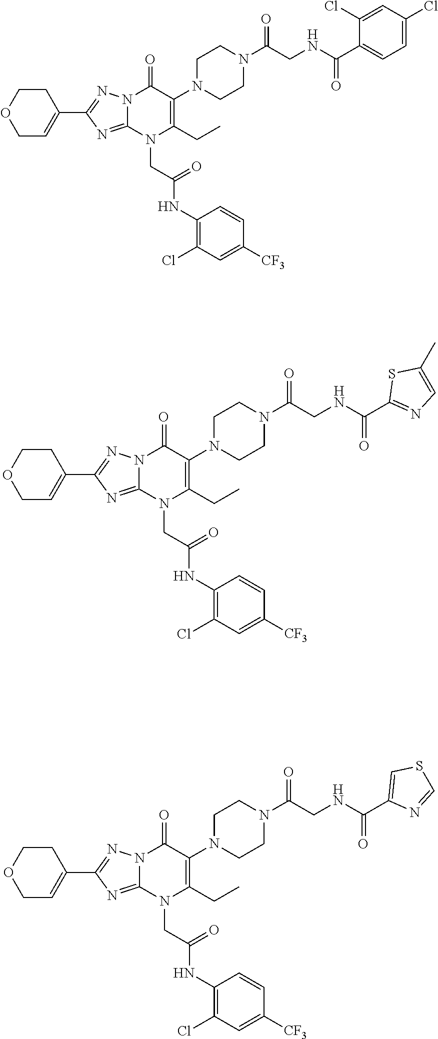 [SMILES: CCc1c(N2CCN(C(=O)CNC(=O)c3ccc(Cl)cc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3ncc(C)s3)CC2)c(=O)n2nc(C3=CCOCC3)nc2n1CC(=O)Nc1ccc(C(F)(F)F)cc1Cl ; CCc1c(N2CCN(C(=O)CNC(=O)c3cscn3)CC2)c(=O)n2nc(C3=CCOCC3)nc2n1CC(=O)Nc1ccc(C(F)(F)F)cc1Cl]

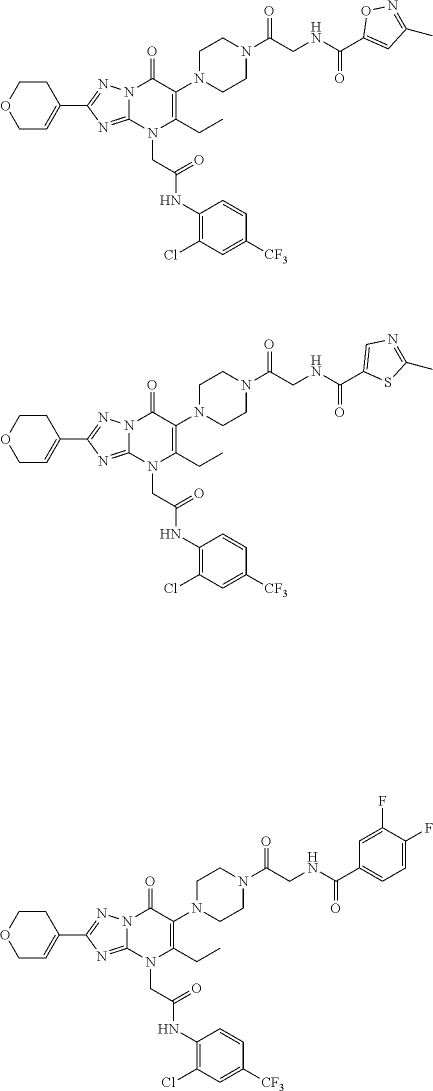 [SMILES: CCc1c(N2CCN(C(=O)CNC(=O)c3cc(C)no3)CC2)c(=O)n2nc(C3=CCOCC3)nc2n1CC(=O)Nc1ccc(C(F)(F)F)cc1Cl ; CCc1c(N2CCN(C(=O)CNC(=O)c3cnc(C)s3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)c(F)c3)CC2)c(=O)n2nc(C3=CCOCC3)nc2n1CC(=O)Nc1ccc(C(F)(F)F)cc1Cl]

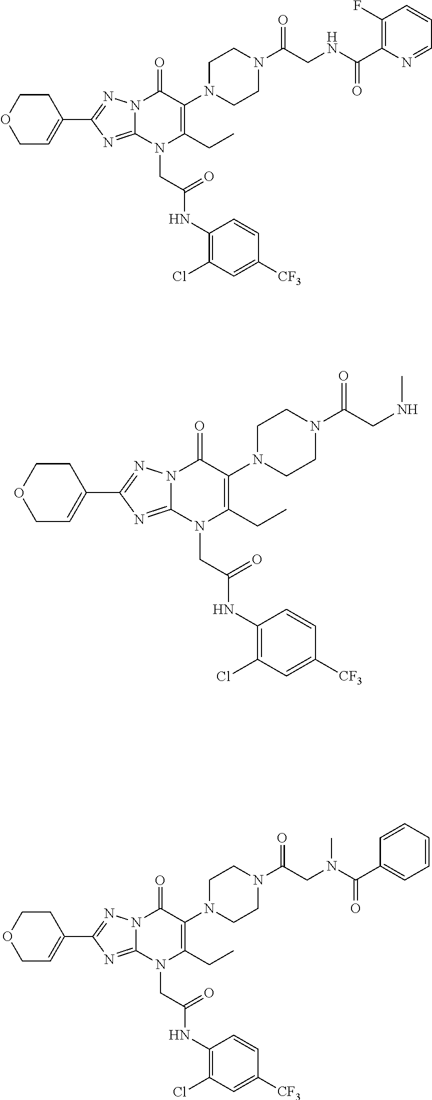 [SMILES: CCc1c(N2CCN(C(=O)CNC(=O)c3ncccc3F)CC2)c(=O)n2nc(C3=CCOCC3)nc2n1CC(=O)Nc1ccc(C(F)(F)F)cc1Cl ; CCc1c(N2CCN(C(=O)CNC)CC2)c(=O)n2nc(C3=CCOCC3)nc2n1CC(=O)Nc1ccc(C(F)(F)F)cc1Cl ; CCc1c(N2CCN(C(=O)CN(C)C(=O)c3ccccc3)CC2)c(=O)n2nc(C3=CCOCC3)nc2n1CC(=O)Nc1ccc(C(F)(F)F)cc1Cl]

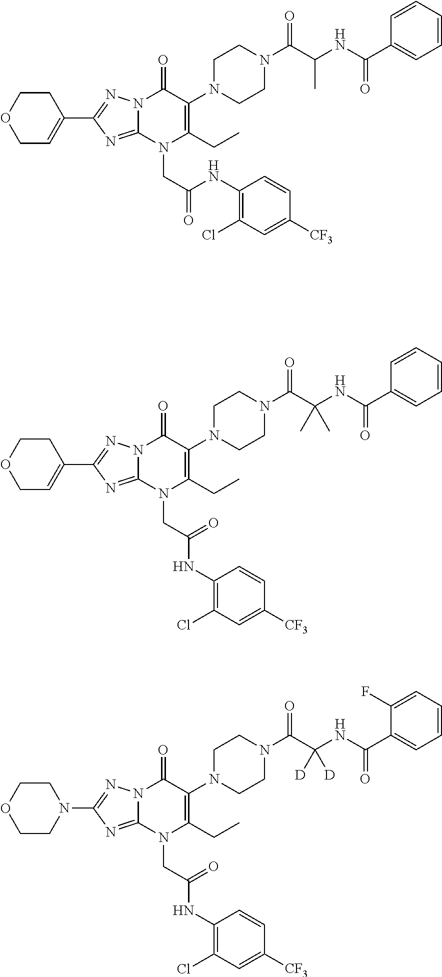 [SMILES: CCc1c(N2CCN(C(=O)C(C)NC(=O)c3ccccc3)CC2)c(=O)n2nc(C3=CCOCC3)nc2n1CC(=O)Nc1ccc(C(F)(F)F)cc1Cl ; CCc1c(N2CCN(C(=O)C(C)(C)NC(=O)c3ccccc3)CC2)c(=O)n2nc(C3=CCOCC3)nc2n1CC(=O)Nc1ccc(C(F)(F)F)cc1Cl ; [2H]C([2H])(NC(=O)c1ccccc1F)C(=O)N1CCN(c2c(CC)n(CC(=O)Nc3ccc(C(F)(F)F)cc3Cl)c3nc(N4CCOCC4)nn3c2=O)CC1]

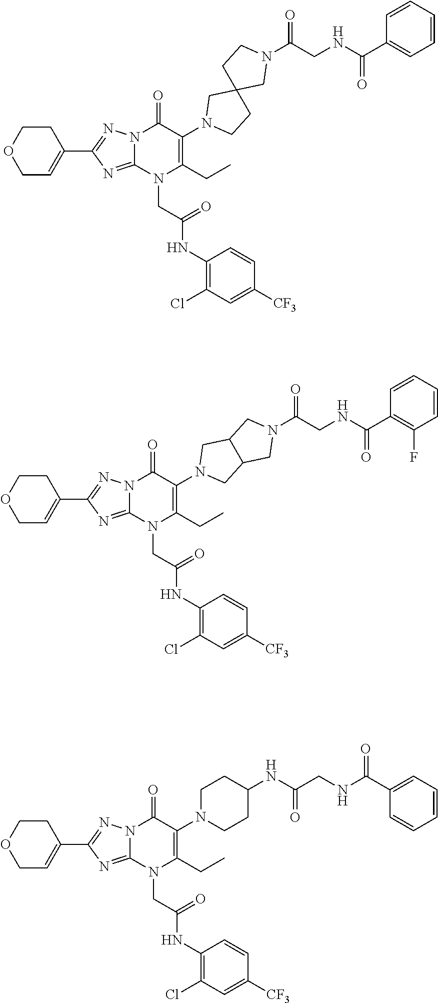 [SMILES: CCc1c(N2CCC3(CCN(C(=O)CNC(=O)c4ccccc4)C3)C2)c(=O)n2nc(C3=CCOCC3)nc2n1CC(=O)Nc1ccc(C(F)(F)F)cc1Cl ; CCc1c(N2CC3CN(C(=O)CNC(=O)c4ccccc4F)CC3C2)c(=O)n2nc(C3=CCOCC3)nc2n1CC(=O)Nc1ccc(C(F)(F)F)cc1Cl ; CCc1c(N2CCC(NC(=O)CNC(=O)c3ccccc3)CC2)c(=O)n2nc(C3=CCOCC3)nc2n1CC(=O)Nc1ccc(C(F)(F)F)cc1Cl]

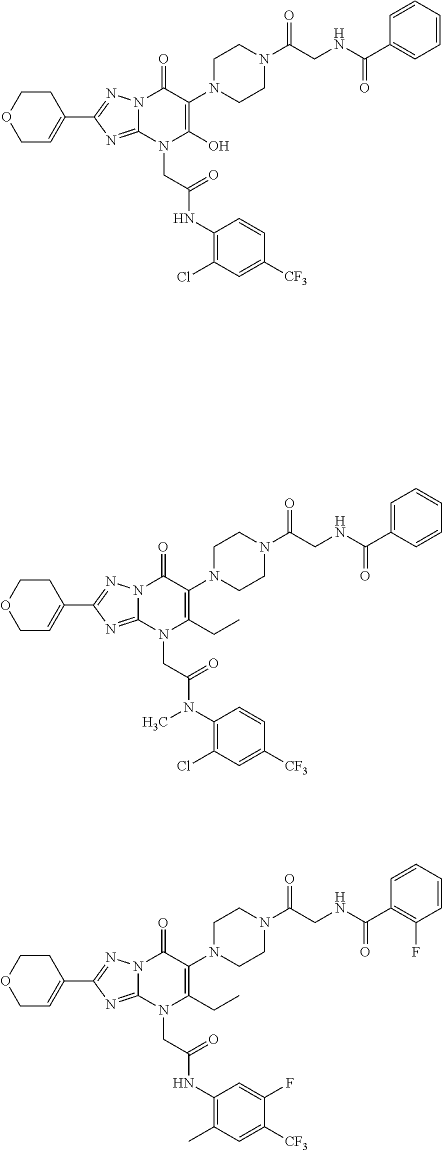 [SMILES: O=C(Cn1c(O)c(N2CCN(C(=O)CNC(=O)c3ccccc3)CC2)c(=O)n2nc(C3=CCOCC3)nc12)Nc1ccc(C(F)(F)F)cc1Cl ; CCc1c(N2CCN(C(=O)CNC(=O)c3ccccc3)CC2)c(=O)n2nc(C3=CCOCC3)nc2n1CC(=O)N(C)c1ccc(C(F)(F)F)cc1Cl ; CCc1c(N2CCN(C(=O)CNC(=O)c3ccccc3F)CC2)c(=O)n2nc(C3=CCOCC3)nc2n1CC(=O)Nc1cc(F)c(C(F)(F)F)cc1C]

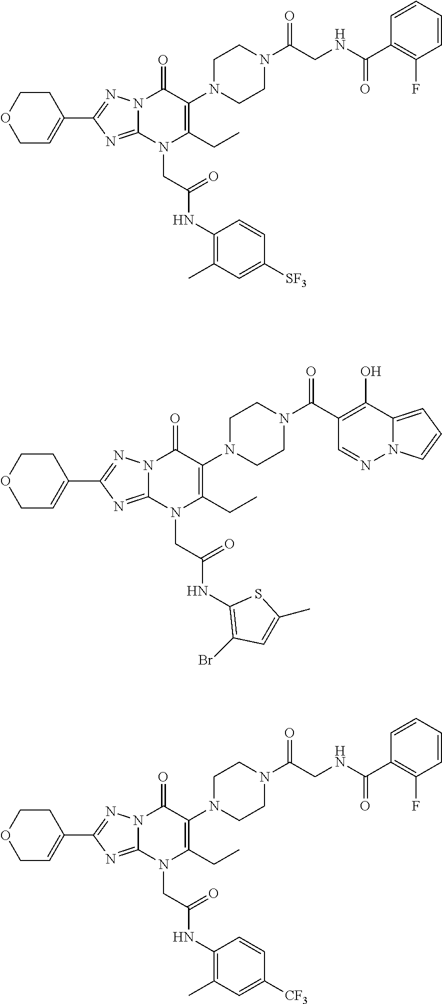

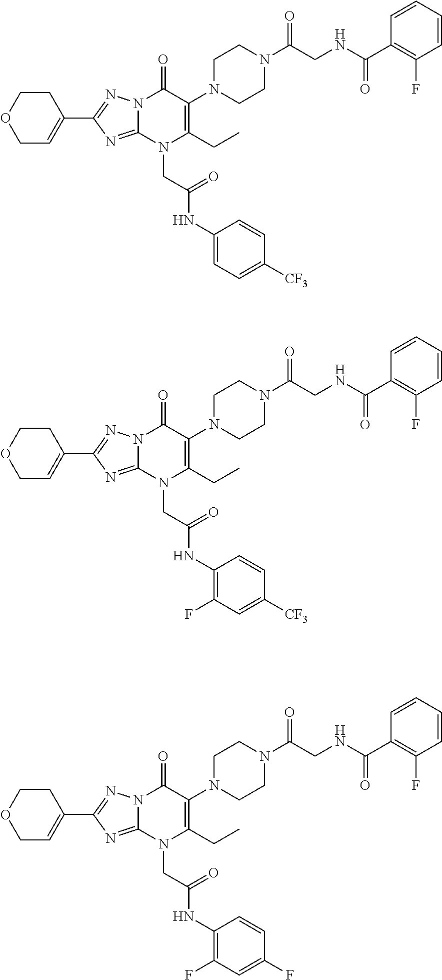 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1 ; CCc1c(N2CCN(C(=O)CNC(=O)c3ccccc3F)CC2)c(=O)n2nc(C3=CCOCC3)nc2n1CC(=O)Nc1ccc(C(F)(F)F)cc1F ; CCc1c(N2CCN(C(=O)CNC(=O)c3ccccc3F)CC2)c(=O)n2nc(C3=CCOCC3)nc2n1CC(=O)Nc1ccc(F)cc1F]

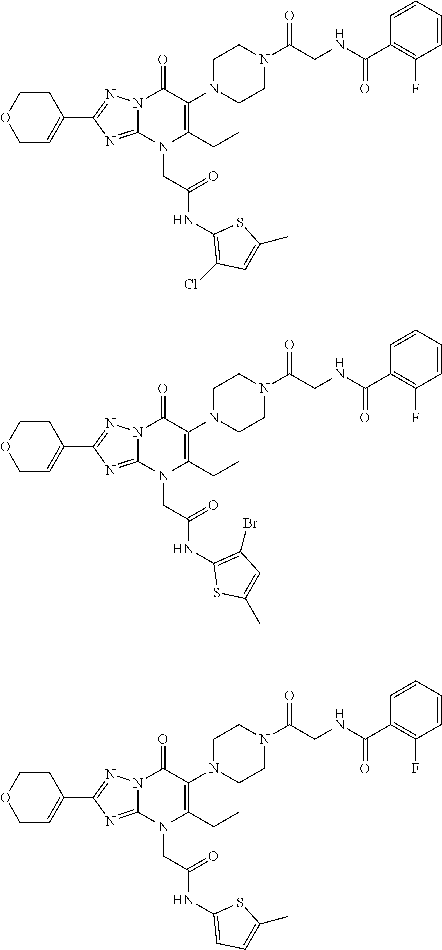 [SMILES: CCc1c(N2CCN(C(=O)CNC(=O)c3ccccc3F)CC2)c(=O)n2nc(C3=CCOCC3)nc2n1CC(=O)Nc1sc(C)cc1Cl ; CCc1c(N2CCN(C(=O)CNC(=O)c3ccccc3F)CC2)c(=O)n2nc(C3=CCOCC3)nc2n1CC(=O)Nc1sc(C)cc1Br ; CCc1c(N2CCN(C(=O)CNC(=O)c3ccccc3F)CC2)c(=O)n2nc(C3=CCOCC3)nc2n1CC(=O)Nc1ccc(C)s1]

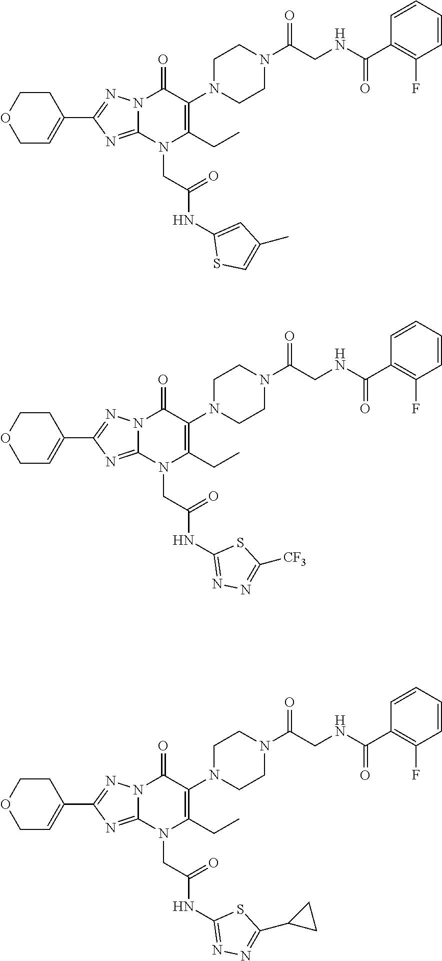 [SMILES: CCc1c(N2CCN(C(=O)CNC(=O)c3ccccc3F)CC2)c(=O)n2nc(C3=CCOCC3)nc2n1CC(=O)Nc1cc(C)cs1 ; CCc1c(N2CCN(C(=O)CNC(=O)c3ccccc3F)CC2)c(=O)n2nc(C3=CCOCC3)nc2n1CC(=O)Nc1nnc(C(F)(F)F)s1 ; CCc1c(N2CCN(C(=O)CNC(=O)c3ccccc3F)CC2)c(=O)n2nc(C3=CCOCC3)nc2n1CC(=O)Nc1nnc(C2CC2)s1]

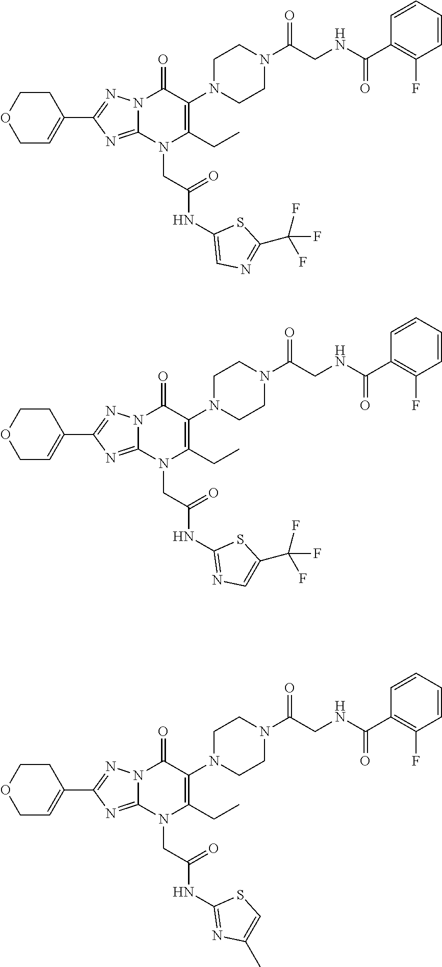 [SMILES: CCc1c(N2CCN(C(=O)CNC(=O)c3ccccc3F)CC2)c(=O)n2nc(C3=CCOCC3)nc2n1CC(=O)Nc1cnc(C(F)(F)F)s1 ; CCc1c(N2CCN(C(=O)CNC(=O)c3ccccc3F)CC2)c(=O)n2nc(C3=CCOCC3)nc2n1CC(=O)Nc1ncc(C(F)(F)F)s1 ; CCc1c(N2CCN(C(=O)CNC(=O)c3ccccc3F)CC2)c(=O)n2nc(C3=CCOCC3)nc2n1CC(=O)Nc1nc(C)cs1]

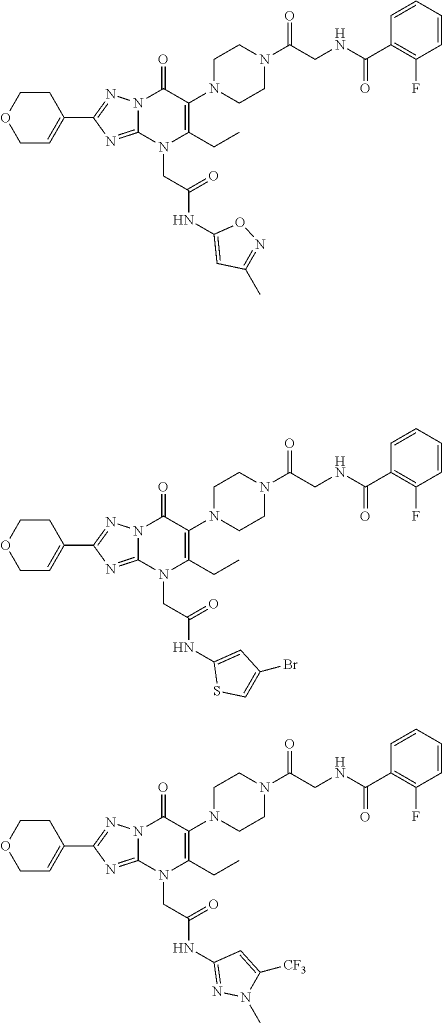 [SMILES: CCc1c(N2CCN(C(=O)CNC(=O)c3ccccc3F)CC2)c(=O)n2nc(C3=CCOCC3)nc2n1CC(=O)Nc1cc(C)no1 ; CCc1c(N2CCN(C(=O)CNC(=O)c3ccccc3F)CC2)c(=O)n2nc(C3=CCOCC3)nc2n1CC(=O)Nc1cc(Br)cs1 ; CCc1c(N2CCN(C(=O)CNC(=O)c3ccccc3F)CC2)c(=O)n2nc(C3=CCOCC3)nc2n1CC(=O)Nc1cc(C(F)(F)F)n(C)n1]

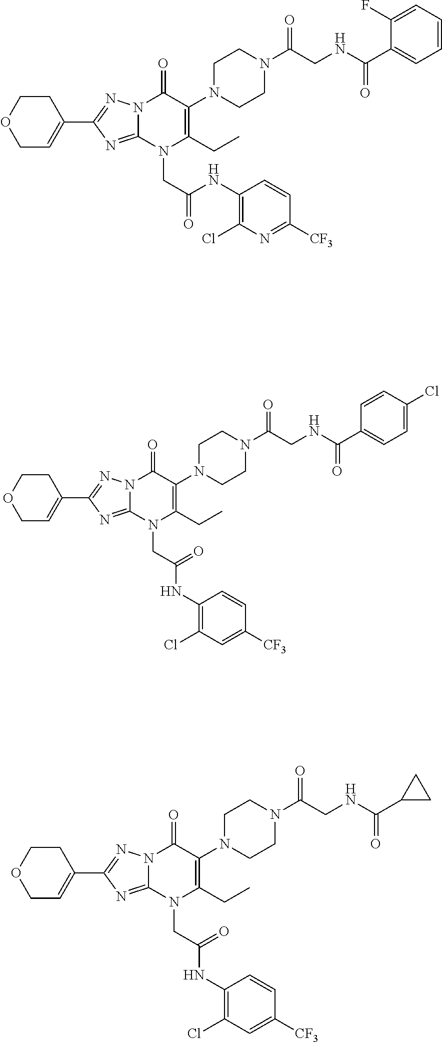 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)nc1Cl ; CCc1c(N2CCN(C(=O)CNC(=O)c3ccc(Cl)cc3)CC2)c(=O)n2nc(C3=CCOCC3)nc2n1CC(=O)Nc1ccc(C(F)(F)F)cc1Cl ; CCc1c(N2CCN(C(=O)CNC(=O)C3CC3)CC2)c(=O)n2nc(C3=CCOCC3)nc2n1CC(=O)Nc1ccc(C(F)(F)F)cc1Cl]

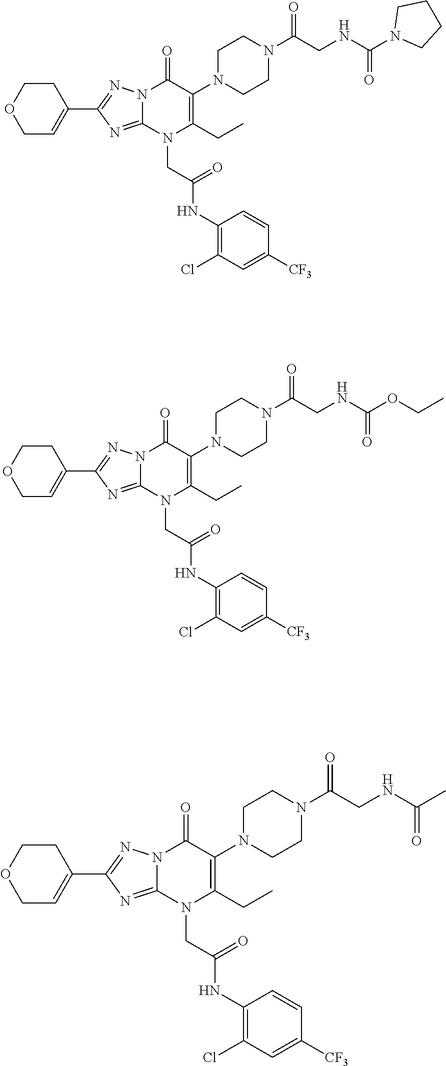 [SMILES: CCc1c(N2CCN(C(=O)CNC(=O)N3CCCC3)CC2)c(=O)n2nc(C3=CCOCC3)nc2n1CC(=O)Nc1ccc(C(F)(F)F)cc1Cl ; CCOC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)CNC(C)=O)CC2)c(=O)n2nc(C3=CCOCC3)nc2n1CC(=O)Nc1ccc(C(F)(F)F)cc1Cl]

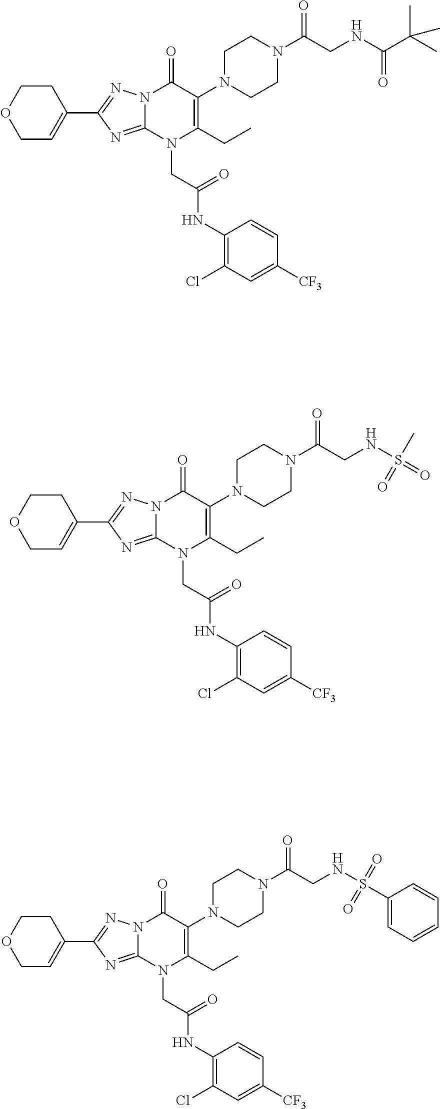 [SMILES: CCc1c(N2CCN(C(=O)CNC(=O)C(C)(C)C)CC2)c(=O)n2nc(C3=CCOCC3)nc2n1CC(=O)Nc1ccc(C(F)(F)F)cc1Cl ; CCc1c(N2CCN(C(=O)CNS(C)(=O)=O)CC2)c(=O)n2nc(C3=CCOCC3)nc2n1CC(=O)Nc1ccc(C(F)(F)F)cc1Cl ; CCc1c(N2CCN(C(=O)CNS(=O)(=O)c3ccccc3)CC2)c(=O)n2nc(C3=CCOCC3)nc2n1CC(=O)Nc1ccc(C(F)(F)F)cc1Cl]

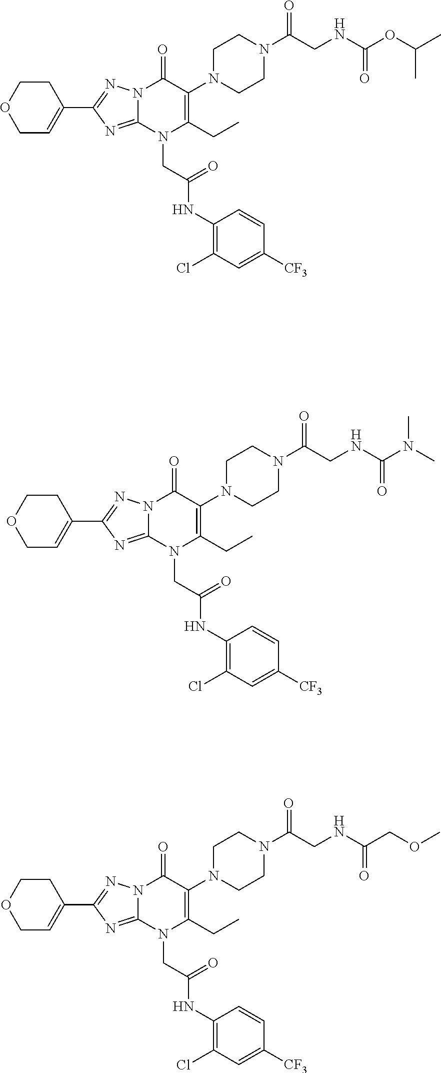 [SMILES: CCc1c(N2CCN(C(=O)CNC(=O)OC(C)C)CC2)c(=O)n2nc(C3=CCOCC3)nc2n1CC(=O)Nc1ccc(C(F)(F)F)cc1Cl ; CCc1c(N2CCN(C(=O)CNC(=O)N(C)C)CC2)c(=O)n2nc(C3=CCOCC3)nc2n1CC(=O)Nc1ccc(C(F)(F)F)cc1Cl ; CCc1c(N2CCN(C(=O)CNC(=O)COC)CC2)c(=O)n2nc(C3=CCOCC3)nc2n1CC(=O)Nc1ccc(C(F)(F)F)cc1Cl]

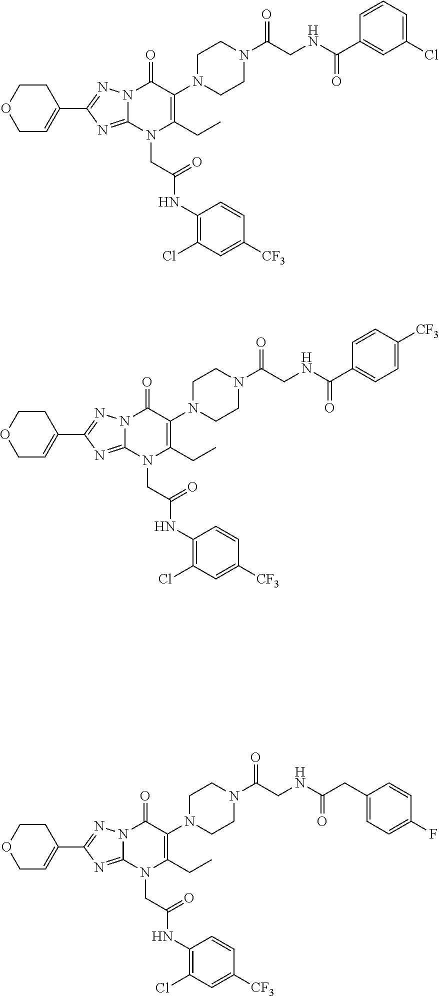 [SMILES: CCc1c(N2CCN(C(=O)CNC(=O)c3cccc(Cl)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(C(F)(F)F)cc3)CC2)c(=O)n2nc(C3=CCOCC3)nc2n1CC(=O)Nc1ccc(C(F)(F)F)cc1Cl ; CCc1c(N2CCN(C(=O)CNC(=O)Cc3ccc(F)cc3)CC2)c(=O)n2nc(C3=CCOCC3)nc2n1CC(=O)Nc1ccc(C(F)(F)F)cc1Cl]

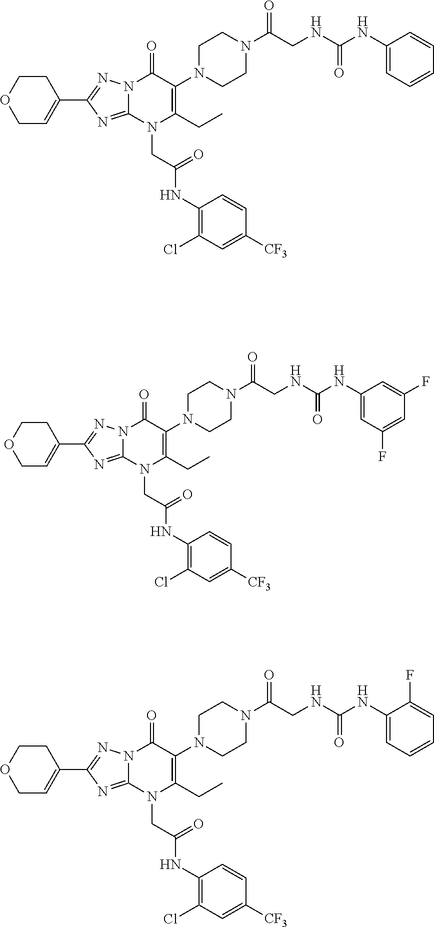 [SMILES: CCc1c(N2CCN(C(=O)CNC(=O)Nc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3cc(F)cc(F)c3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3ccccc3F)CC2)c(=O)n2nc(C3=CCOCC3)nc2n1CC(=O)Nc1ccc(C(F)(F)F)cc1Cl]

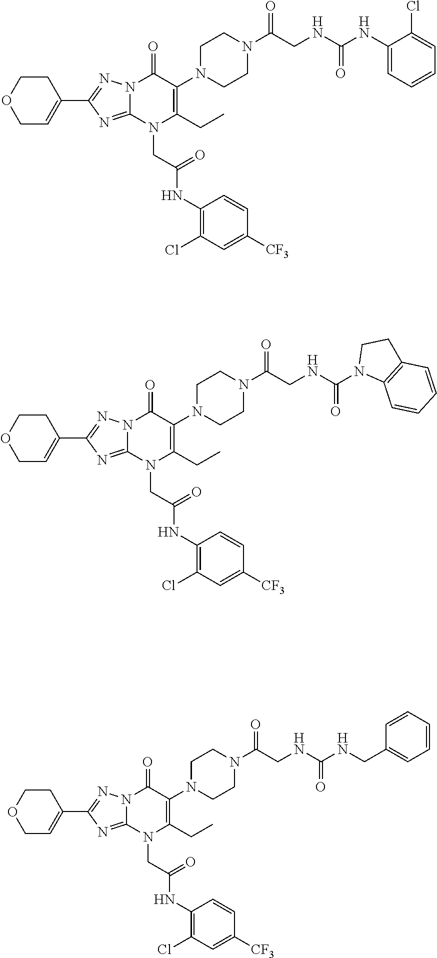 [SMILES: CCc1c(N2CCN(C(=O)CNC(=O)Nc3ccccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)N3CCc4ccccc43)CC2)c(=O)n2nc(C3=CCOCC3)nc2n1CC(=O)Nc1ccc(C(F)(F)F)cc1Cl ; CCc1c(N2CCN(C(=O)CNC(=O)NCc3ccccc3)CC2)c(=O)n2nc(C3=CCOCC3)nc2n1CC(=O)Nc1ccc(C(F)(F)F)cc1Cl]

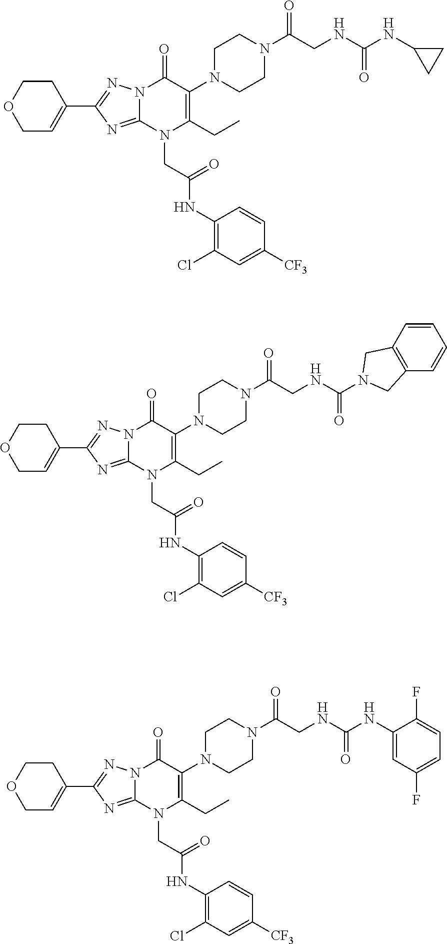 [SMILES: CCc1c(N2CCN(C(=O)CNC(=O)NC3CC3)CC2)c(=O)n2nc(C3=CCOCC3)nc2n1CC(=O)Nc1ccc(C(F)(F)F)cc1Cl ; CCc1c(N2CCN(C(=O)CNC(=O)N3Cc4ccccc4C3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3cc(F)ccc3F)CC2)c(=O)n2nc(C3=CCOCC3)nc2n1CC(=O)Nc1ccc(C(F)(F)F)cc1Cl]

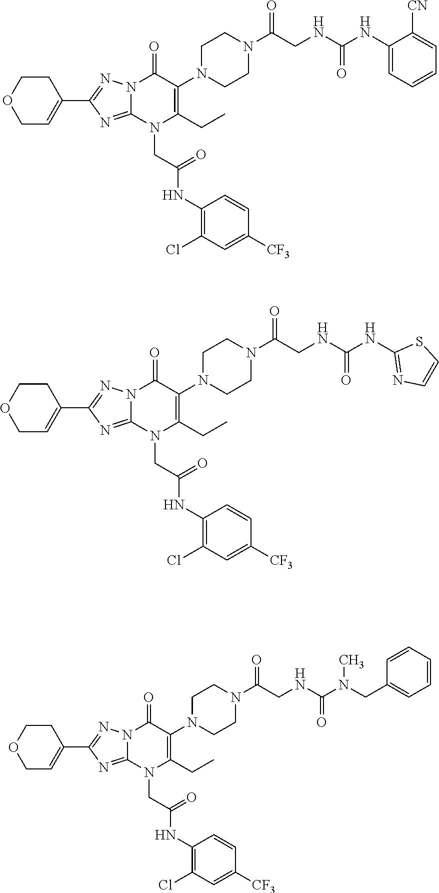 [SMILES: CCc1c(N2CCN(C(=O)CNC(=O)Nc3ccccc3C#N)CC2)c(=O)n2nc(C3=CCOCC3)nc2n1CC(=O)Nc1ccc(C(F)(F)F)cc1Cl ; CCc1c(N2CCN(C(=O)CNC(=O)Nc3nccs3)CC2)c(=O)n2nc(C3=CCOCC3)nc2n1CC(=O)Nc1ccc(C(F)(F)F)cc1Cl ; CCc1c(N2CCN(C(=O)CNC(=O)N(C)Cc3ccccc3)CC2)c(=O)n2nc(C3=CCOCC3)nc2n1CC(=O)Nc1ccc(C(F)(F)F)cc1Cl]

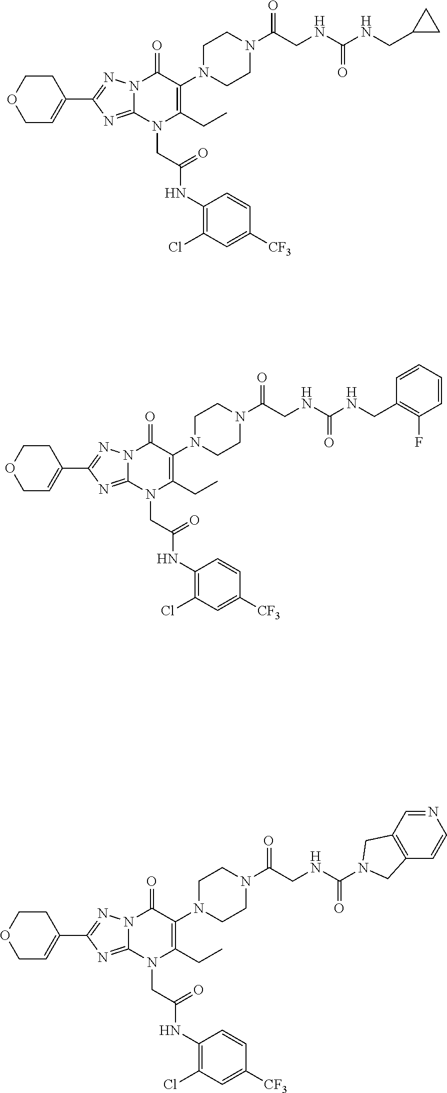 [SMILES: CCc1c(N2CCN(C(=O)CNC(=O)NCC3CC3)CC2)c(=O)n2nc(C3=CCOCC3)nc2n1CC(=O)Nc1ccc(C(F)(F)F)cc1Cl ; CCc1c(N2CCN(C(=O)CNC(=O)NCc3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)N3Cc4ccncc4C3)CC2)c(=O)n2nc(C3=CCOCC3)nc2n1CC(=O)Nc1ccc(C(F)(F)F)cc1Cl]

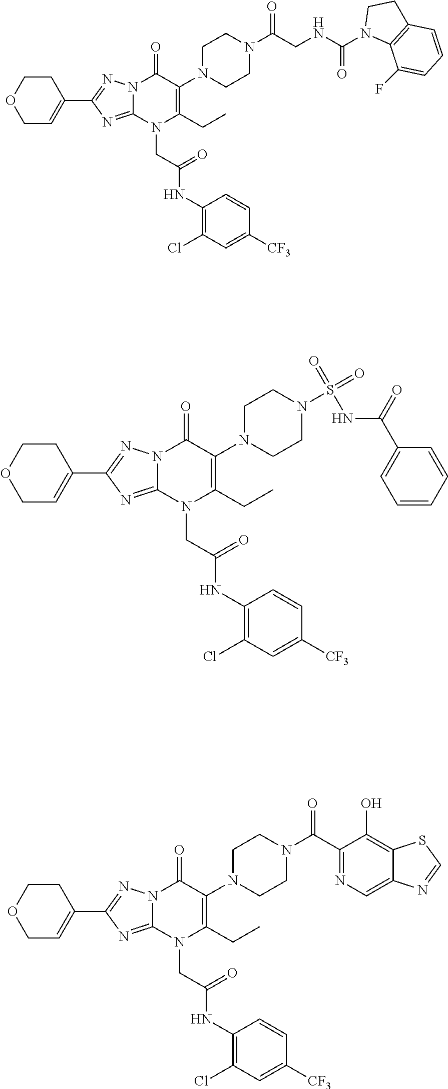 [SMILES: CCc1c(N2CCN(C(=O)CNC(=O)N3CCc4cccc(F)c43)CC2)c(=O)n2nc(C3=CCOCC3)nc2n1CC(=O)Nc1ccc(C(F)(F)F)cc1Cl ; CCc1c(N2CCN(S(=O)(=O)NC(=O)c3ccccc3)CC2)c(=O)n2nc(C3=CCOCC3)nc2n1CC(=O)Nc1ccc(C(F)(F)F)cc1Cl ; CCc1c(N2CCN(C(=O)c3ncc4ncsc4c3O)CC2)c(=O)n2nc(C3=CCOCC3)nc2n1CC(=O)Nc1ccc(C(F)(F)F)cc1Cl]

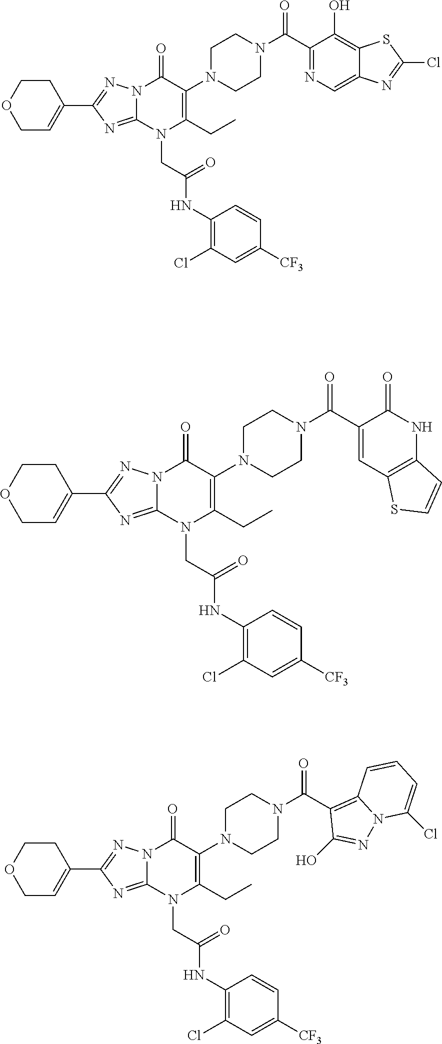 [SMILES: CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1Cl ; CCc1c(N2CCN(C(=O)c3cc4sccc4[nH]c3=O)CC2)c(=O)n2nc(C3=CCOCC3)nc2n1CC(=O)Nc1ccc(C(F)(F)F)cc1Cl ; CCc1c(N2CCN(C(=O)c3c(O)nn4c(Cl)cccc34)CC2)c(=O)n2nc(C3=CCOCC3)nc2n1CC(=O)Nc1ccc(C(F)(F)F)cc1Cl]

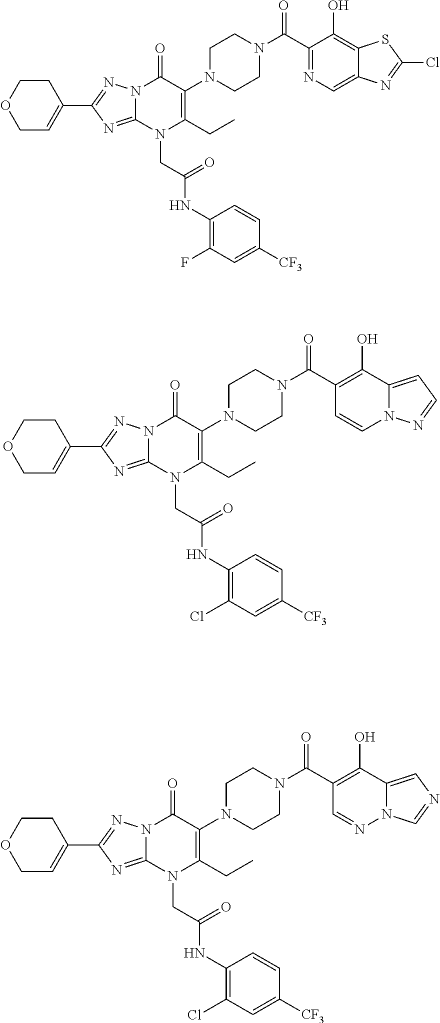 [SMILES: CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1F ; CCc1c(N2CCN(C(=O)c3ccn4nccc4c3O)CC2)c(=O)n2nc(C3=CCOCC3)nc2n1CC(=O)Nc1ccc(C(F)(F)F)cc1Cl ; CCc1c(N2CCN(C(=O)c3cnn4cncc4c3O)CC2)c(=O)n2nc(C3=CCOCC3)nc2n1CC(=O)Nc1ccc(C(F)(F)F)cc1Cl]

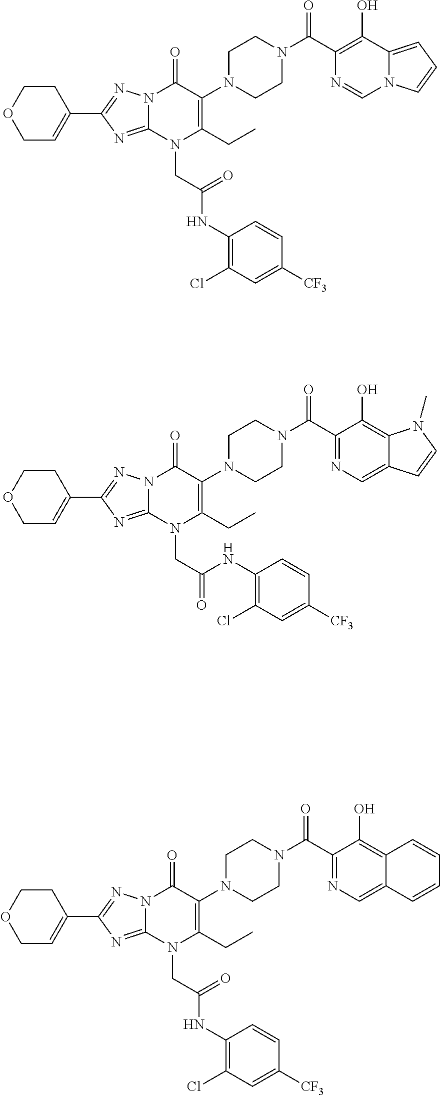 [SMILES: CCc1c(N2CCN(C(=O)c3ncn4cccc4c3O)CC2)c(=O)n2nc(C3=CCOCC3)nc2n1CC(=O)Nc1ccc(C(F)(F)F)cc1Cl ; CCc1c(N2CCN(C(=O)c3ncc4ccn(C)c4c3O)CC2)c(=O)n2nc(C3=CCOCC3)nc2n1CC(=O)Nc1ccc(C(F)(F)F)cc1Cl ; CCc1c(N2CCN(C(=O)c3ncc4ccccc4c3O)CC2)c(=O)n2nc(C3=CCOCC3)nc2n1CC(=O)Nc1ccc(C(F)(F)F)cc1Cl]

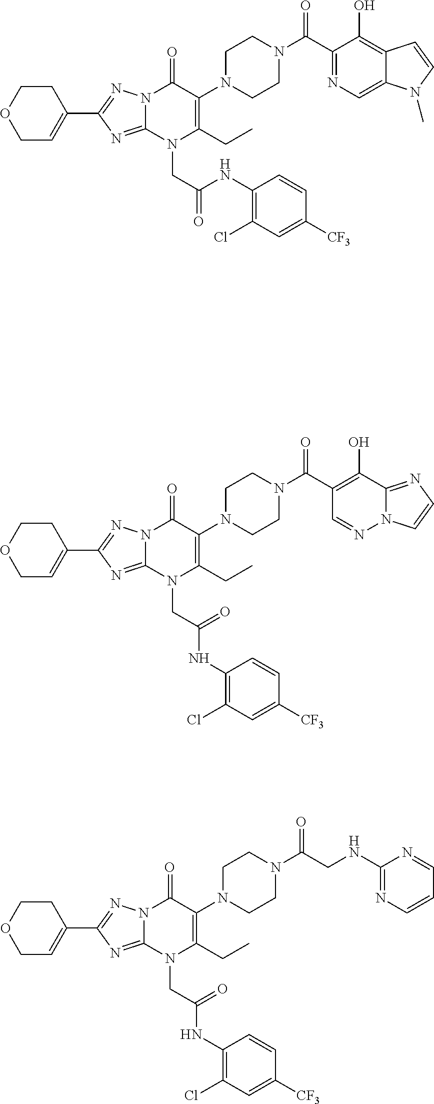 [SMILES: CCc1c(N2CCN(C(=O)c3ncc4c(ccn4C)c3O)CC2)c(=O)n2nc(C3=CCOCC3)nc2n1CC(=O)Nc1ccc(C(F)(F)F)cc1Cl ; CCc1c(N2CCN(C(=O)c3cnn4ccnc4c3O)CC2)c(=O)n2nc(C3=CCOCC3)nc2n1CC(=O)Nc1ccc(C(F)(F)F)cc1Cl ; CCc1c(N2CCN(C(=O)CNc3ncccn3)CC2)c(=O)n2nc(C3=CCOCC3)nc2n1CC(=O)Nc1ccc(C(F)(F)F)cc1Cl]

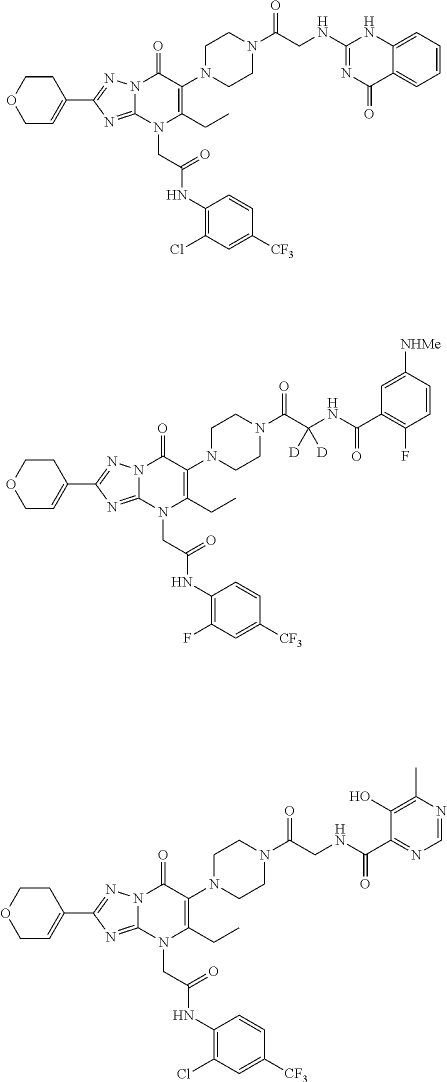 [SMILES: CCc1c(N2CCN(C(=O)CNc3nc(=O)c4ccccc4[nH]3)CC2)c(=O)n2nc(C3=CCOCC3)nc2n1CC(=O)Nc1ccc(C(F)(F)F)cc1Cl ; [2H]C([2H])(NC(=O)c1cc(NC)ccc1F)C(=O)N1CCN(c2c(CC)n(CC(=O)Nc3ccc(C(F)(F)F)cc3F)c3nc(C4=CCOCC4)nn3c2=O)CC1 ; CCc1c(N2CCN(C(=O)CNC(=O)c3ncnc(C)c3O)CC2)c(=O)n2nc(C3=CCOCC3)nc2n1CC(=O)Nc1ccc(C(F)(F)F)cc1Cl]

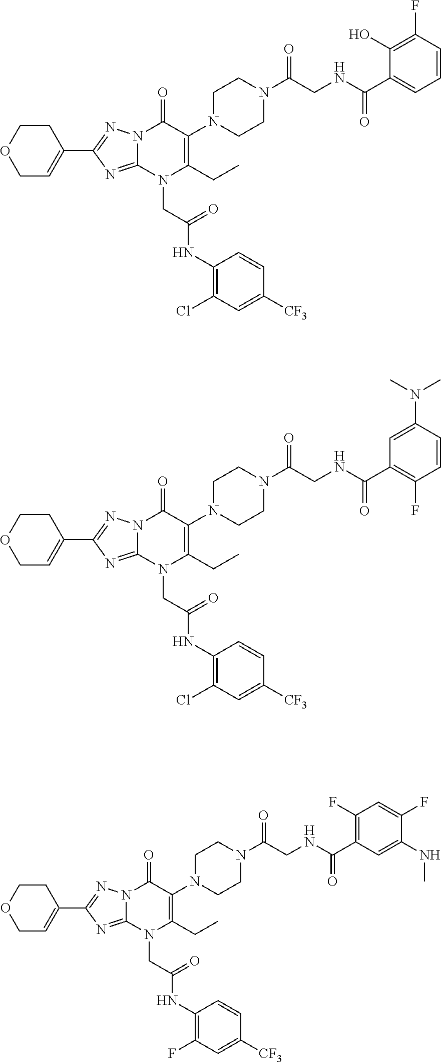 [SMILES: CCc1c(N2CCN(C(=O)CNC(=O)c3cccc(F)c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(N(C)C)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(NC)c(F)cc3F)CC2)c(=O)n2nc(C3=CCOCC3)nc2n1CC(=O)Nc1ccc(C(F)(F)F)cc1F]

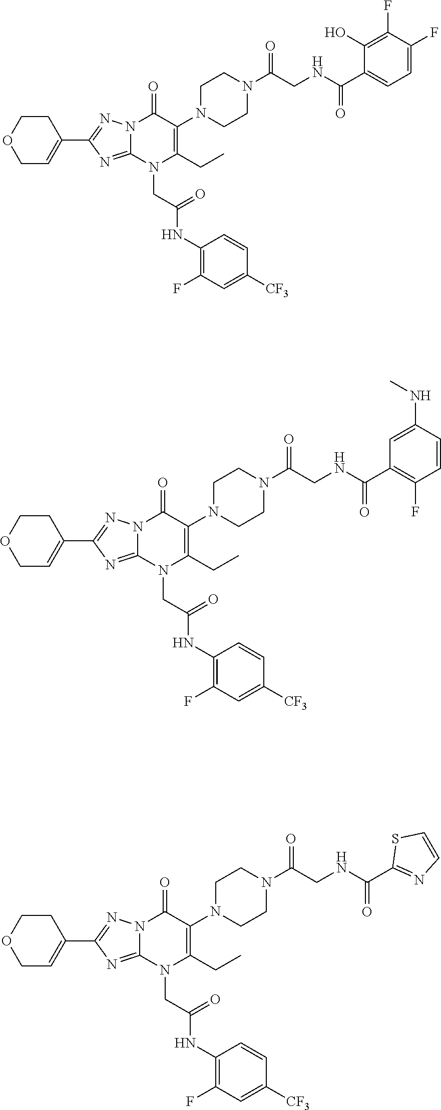 [SMILES: CCc1c(N2CCN(C(=O)CNC(=O)c3ccc(F)c(F)c3O)CC2)c(=O)n2nc(C3=CCOCC3)nc2n1CC(=O)Nc1ccc(C(F)(F)F)cc1F ; CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1F ; CCc1c(N2CCN(C(=O)CNC(=O)c3nccs3)CC2)c(=O)n2nc(C3=CCOCC3)nc2n1CC(=O)Nc1ccc(C(F)(F)F)cc1F]

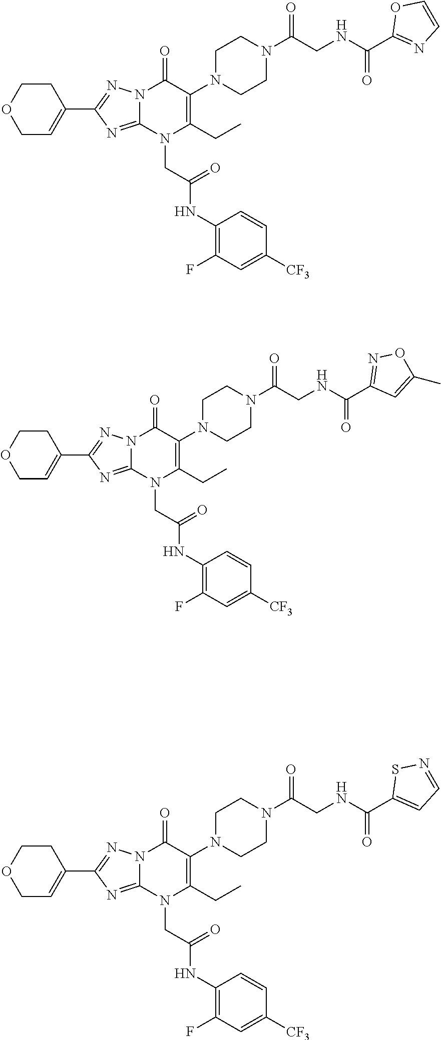 [SMILES: CCc1c(N2CCN(C(=O)CNC(=O)c3ncco3)CC2)c(=O)n2nc(C3=CCOCC3)nc2n1CC(=O)Nc1ccc(C(F)(F)F)cc1F ; CCc1c(N2CCN(C(=O)CNC(=O)c3cc(C)on3)CC2)c(=O)n2nc(C3=CCOCC3)nc2n1CC(=O)Nc1ccc(C(F)(F)F)cc1F ; CCc1c(N2CCN(C(=O)CNC(=O)c3ccns3)CC2)c(=O)n2nc(C3=CCOCC3)nc2n1CC(=O)Nc1ccc(C(F)(F)F)cc1F]

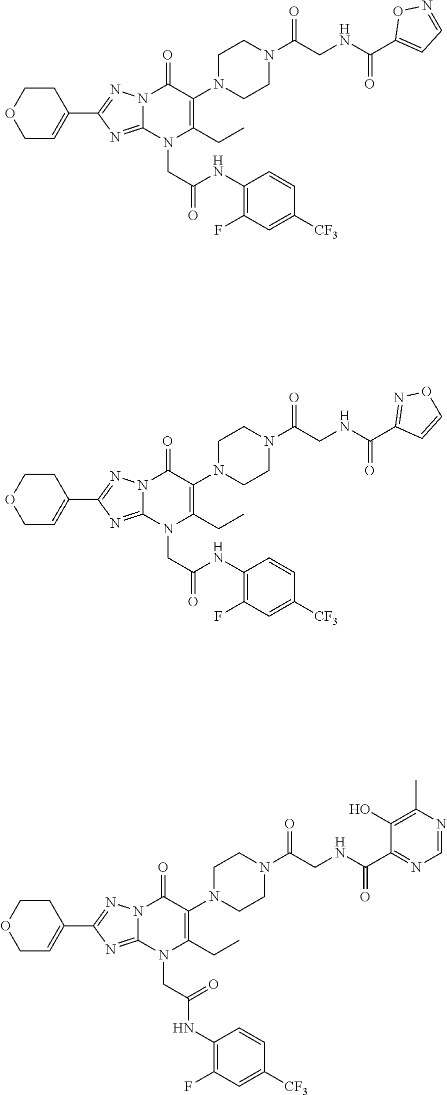 [SMILES: CCc1c(N2CCN(C(=O)CNC(=O)c3ccno3)CC2)c(=O)n2nc(C3=CCOCC3)nc2n1CC(=O)Nc1ccc(C(F)(F)F)cc1F ; CCc1c(N2CCN(C(=O)CNC(=O)c3ccon3)CC2)c(=O)n2nc(C3=CCOCC3)nc2n1CC(=O)Nc1ccc(C(F)(F)F)cc1F ; CCc1c(N2CCN(C(=O)CNC(=O)c3ncnc(C)c3O)CC2)c(=O)n2nc(C3=CCOCC3)nc2n1CC(=O)Nc1ccc(C(F)(F)F)cc1F]

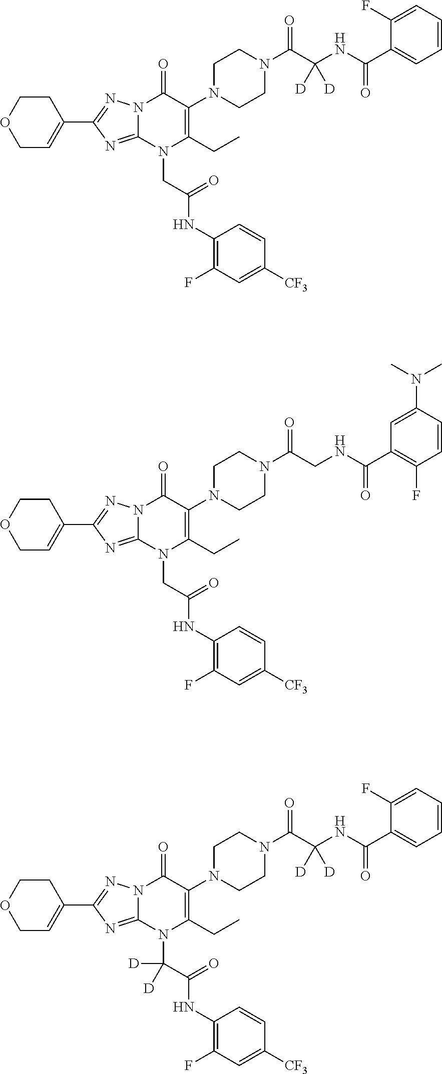 [SMILES: [2H]C([2H])(NC(=O)c1ccccc1F)C(=O)N1CCN(c2c(CC)n(CC(=O)Nc3ccc(C(F)(F)F)cc3F)c3nc(C4=CCOCC4)nn3c2=O)CC1 ; CCc1c(N2CCN(C(=O)CNC(=O)c3cc(N(C)C)ccc3F)CC2)c(=O)n2nc(C3=CCOCC3)nc2n1CC(=O)Nc1ccc(C(F)(F)F)cc1F ; [2H]C([2H])(NC(=O)c1ccccc1F)C(=O)N1CCN(c2c(CC)n(C([2H])([2H])C(=O)Nc3ccc(C(F)(F)F)cc3F)c3nc(C4=CCOCC4)nn3c2=O)CC1]

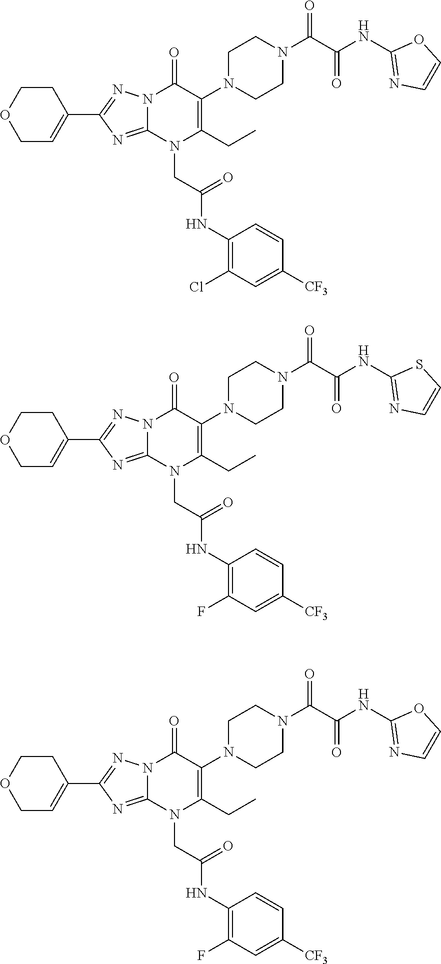 [SMILES: CCc1c(N2CCN(C(=O)C(=O)Nc3ncco3)CC2)c(=O)n2nc(C3=CCOCC3)nc2n1CC(=O)Nc1ccc(C(F)(F)F)cc1Cl ; CCc1c(N2CCN(C(=O)C(=O)Nc3nccs3)CC2)c(=O)n2nc(C3=CCOCC3)nc2n1CC(=O)Nc1ccc(C(F)(F)F)cc1F ; CCc1c(N2CCN(C(=O)C(=O)Nc3ncco3)CC2)c(=O)n2nc(C3=CCOCC3)nc2n1CC(=O)Nc1ccc(C(F)(F)F)cc1F]

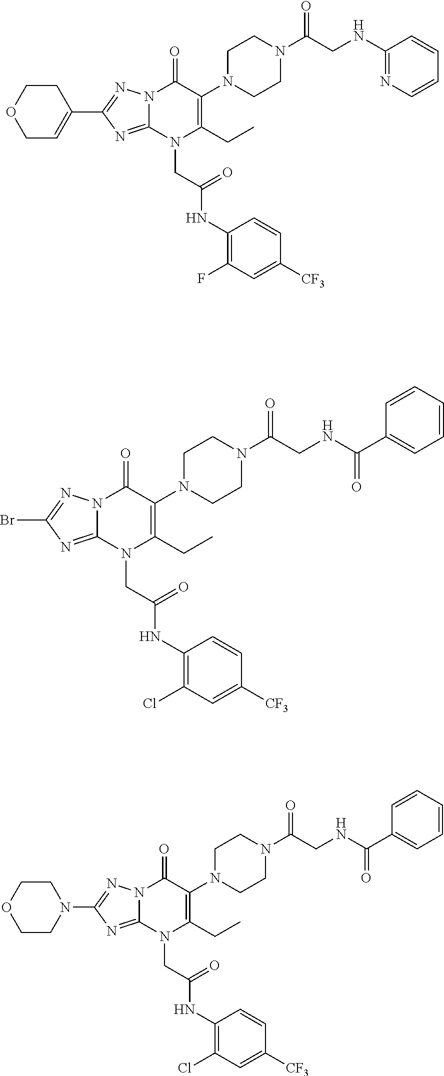 [SMILES: CCc1c(N2CCN(C(=O)CNc3ccccn3)CC2)c(=O)n2nc(C3=CCOCC3)nc2n1CC(=O)Nc1ccc(C(F)(F)F)cc1F ; CCc1c(N2CCN(C(=O)CNC(=O)c3ccccc3)CC2)c(=O)n2nc(Br)nc2n1CC(=O)Nc1ccc(C(F)(F)F)cc1Cl ; CCc1c(N2CCN(C(=O)CNC(=O)c3ccccc3)CC2)c(=O)n2nc(N3CCOCC3)nc2n1CC(=O)Nc1ccc(C(F)(F)F)cc1Cl]

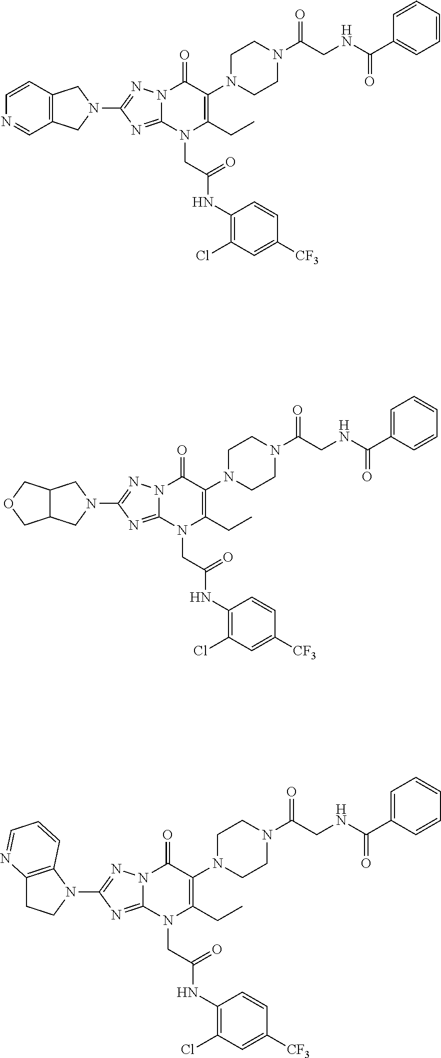 [SMILES: CCc1c(N2CCN(C(=O)CNC(=O)c3ccccc3)CC2)c(=O)n2nc(N3Cc4ccncc4C3)nc2n1CC(=O)Nc1ccc(C(F)(F)F)cc1Cl ; CCc1c(N2CCN(C(=O)CNC(=O)c3ccccc3)CC2)c(=O)n2nc(N3CC4COCC4C3)nc2n1CC(=O)Nc1ccc(C(F)(F)F)cc1Cl ; CCc1c(N2CCN(C(=O)CNC(=O)c3ccccc3)CC2)c(=O)n2nc(N3CCc4ncccc43)nc2n1CC(=O)Nc1ccc(C(F)(F)F)cc1Cl]

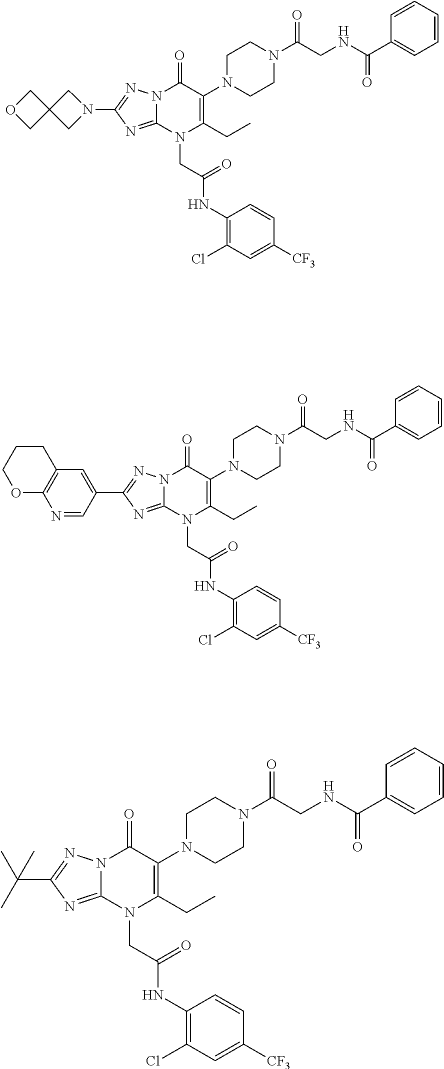 [SMILES: CCc1c(N2CCN(C(=O)CNC(=O)c3ccccc3)CC2)c(=O)n2nc(N3CC4(COC4)C3)nc2n1CC(=O)Nc1ccc(C(F)(F)F)cc1Cl ; CCc1c(N2CCN(C(=O)CNC(=O)c3ccccc3)CC2)c(=O)n2nc(-c3cnc4c(c3)CCCO4)nc2n1CC(=O)Nc1ccc(C(F)(F)F)cc1Cl ; CCc1c(N2CCN(C(=O)CNC(=O)c3ccccc3)CC2)c(=O)n2nc(C(C)(C)C)nc2n1CC(=O)Nc1ccc(C(F)(F)F)cc1Cl]

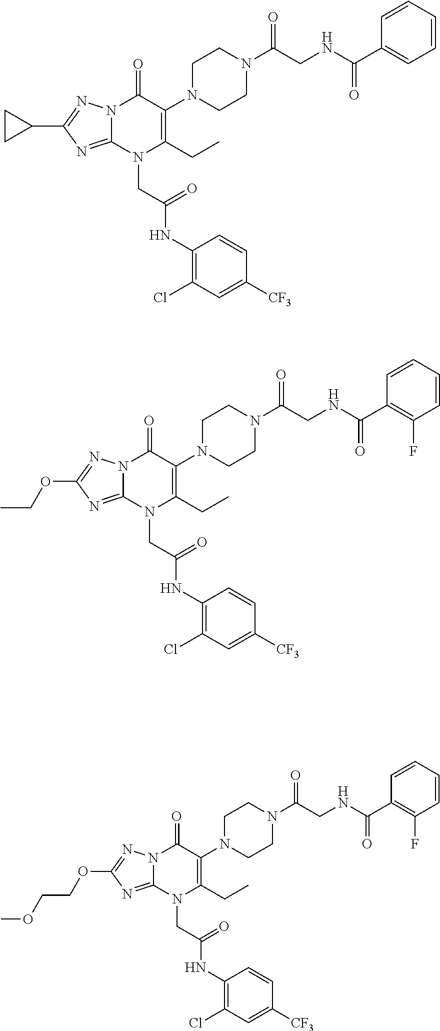 [SMILES: CCc1c(N2CCN(C(=O)CNC(=O)c3ccccc3)CC2)c(=O)n2nc(C3CC3)nc2n1CC(=O)Nc1ccc(C(F)(F)F)cc1Cl ; CCOc1nc2n(CC(=O)Nc3ccc(C(F)(F)F)cc3Cl)c(CC)c(N3CCN(C(=O)CNC(=O)c4ccccc4F)CC3)c(=O)n2n1 ; CCc1c(N2CCN(C(=O)CNC(=O)c3ccccc3F)CC2)c(=O)n2nc(OCCOC)nc2n1CC(=O)Nc1ccc(C(F)(F)F)cc1Cl]

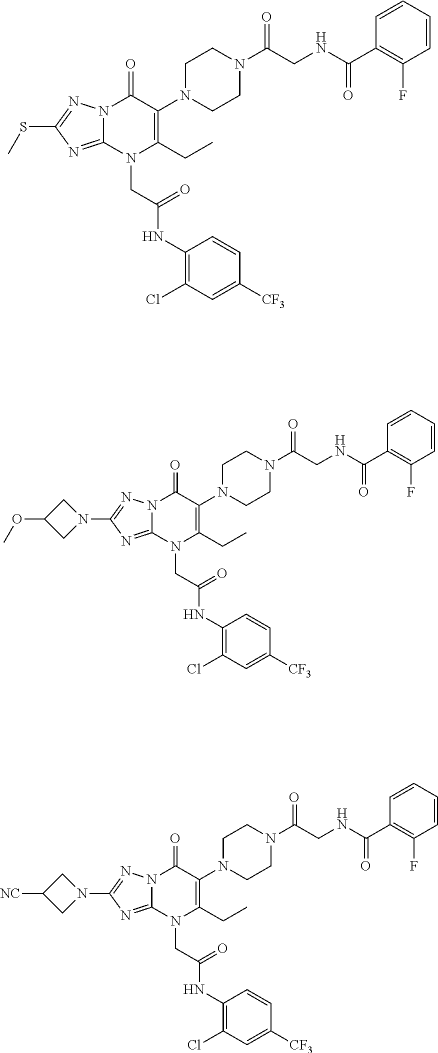 [SMILES: CCc1c(N2CCN(C(=O)CNC(=O)c3ccccc3F)CC2)c(=O)n2nc(SC)nc2n1CC(=O)Nc1ccc(C(F)(F)F)cc1Cl ; CCc1c(N2CCN(C(=O)CNC(=O)c3ccccc3F)CC2)c(=O)n2nc(N3CC(OC)C3)nc2n1CC(=O)Nc1ccc(C(F)(F)F)cc1Cl ; CCc1c(N2CCN(C(=O)CNC(=O)c3ccccc3F)CC2)c(=O)n2nc(N3CC(C#N)C3)nc2n1CC(=O)Nc1ccc(C(F)(F)F)cc1Cl]

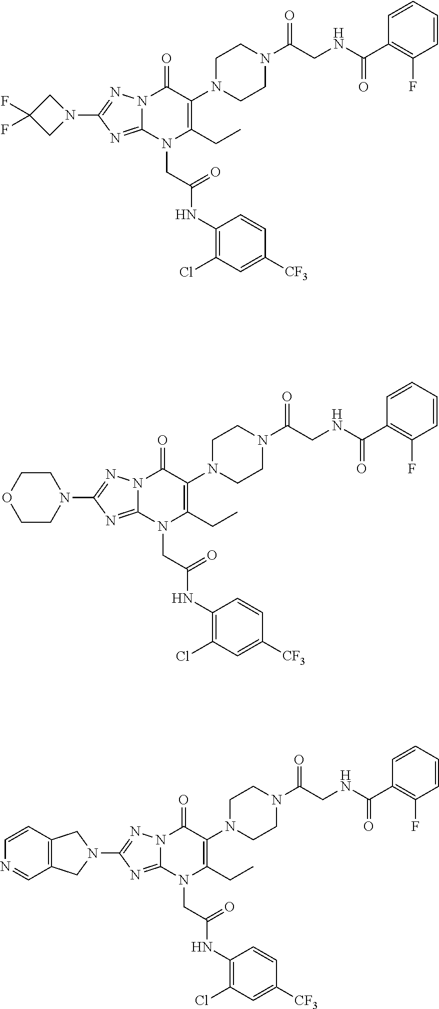 [SMILES: CCc1c(N2CCN(C(=O)CNC(=O)c3ccccc3F)CC2)c(=O)n2nc(N3CC(F)(F)C3)nc2n1CC(=O)Nc1ccc(C(F)(F)F)cc1Cl ; CCc1c(N2CCN(C(=O)CNC(=O)c3ccccc3F)CC2)c(=O)n2nc(N3CCOCC3)nc2n1CC(=O)Nc1ccc(C(F)(F)F)cc1Cl ; CCc1c(N2CCN(C(=O)CNC(=O)c3ccccc3F)CC2)c(=O)n2nc(N3Cc4ccncc4C3)nc2n1CC(=O)Nc1ccc(C(F)(F)F)cc1Cl]

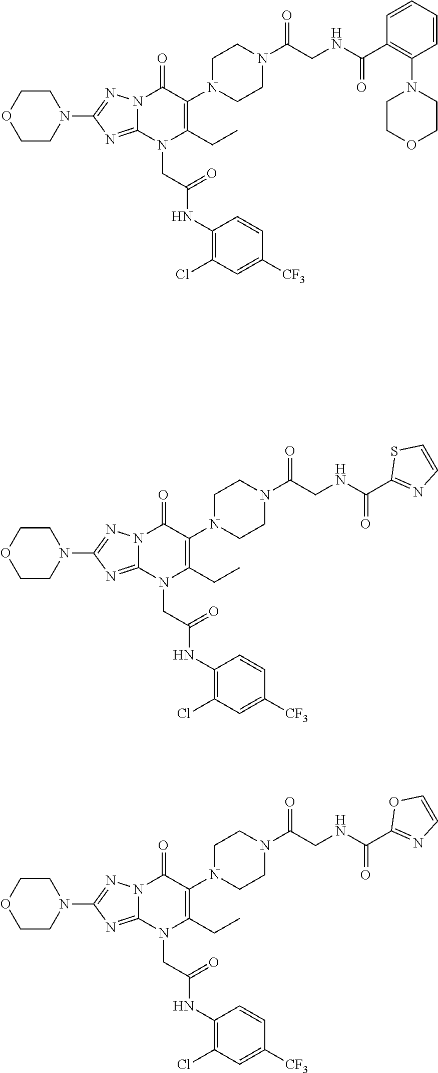 [SMILES: CCc1c(N2CCN(C(=O)CNC(=O)c3ccccc3N3CCOCC3)CC2)c(=O)n2nc(N3CCOCC3)nc2n1CC(=O)Nc1ccc(C(F)(F)F)cc1Cl ; CCc1c(N2CCN(C(=O)CNC(=O)c3nccs3)CC2)c(=O)n2nc(N3CCOCC3)nc2n1CC(=O)Nc1ccc(C(F)(F)F)cc1Cl ; CCc1c(N2CCN(C(=O)CNC(=O)c3ncco3)CC2)c(=O)n2nc(N3CCOCC3)nc2n1CC(=O)Nc1ccc(C(F)(F)F)cc1Cl]

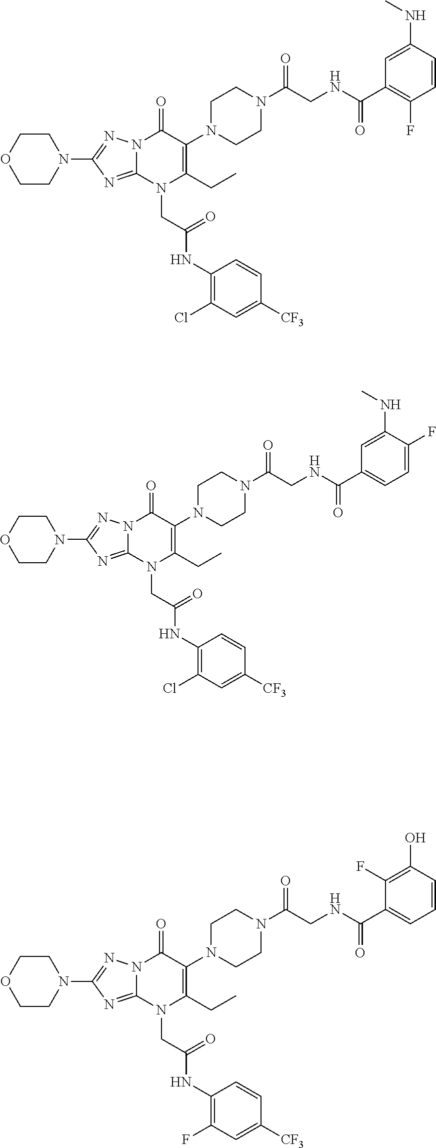 [SMILES: CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1Cl ; CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1Cl ; CCc1c(N2CCN(C(=O)CNC(=O)c3cccc(O)c3F)CC2)c(=O)n2nc(N3CCOCC3)nc2n1CC(=O)Nc1ccc(C(F)(F)F)cc1F]

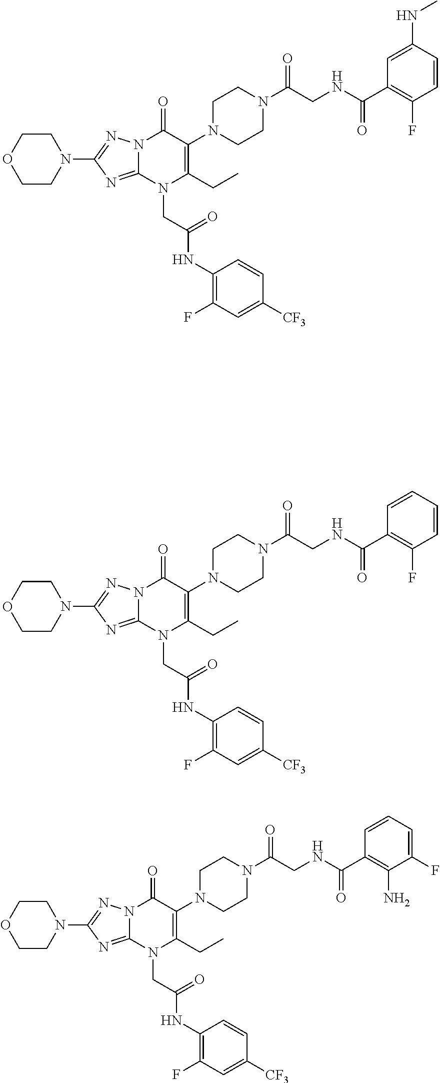 [SMILES: CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1F ; CCc1c(N2CCN(C(=O)CNC(=O)c3ccccc3F)CC2)c(=O)n2nc(N3CCOCC3)nc2n1CC(=O)Nc1ccc(C(F)(F)F)cc1F ; CCc1c(N2CCN(C(=O)CNC(=O)c3cccc(F)c3N)CC2)c(=O)n2nc(N3CCOCC3)nc2n1CC(=O)Nc1ccc(C(F)(F)F)cc1F]

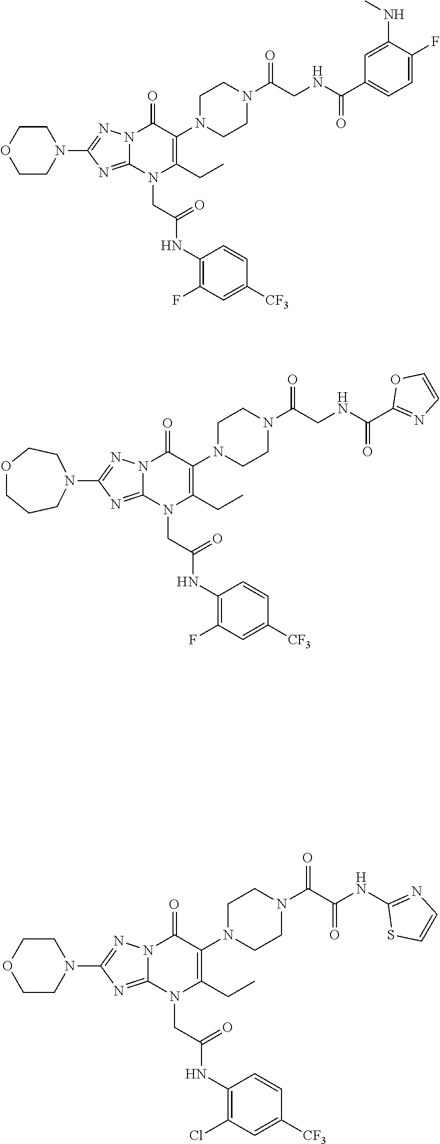 [SMILES: CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1F ; CCc1c(N2CCN(C(=O)CNC(=O)c3ncco3)CC2)c(=O)n2nc(N3CCCOCC3)nc2n1CC(=O)Nc1ccc(C(F)(F)F)cc1F ; CCc1c(N2CCN(C(=O)C(=O)Nc3nccs3)CC2)c(=O)n2nc(N3CCOCC3)nc2n1CC(=O)Nc1ccc(C(F)(F)F)cc1Cl]

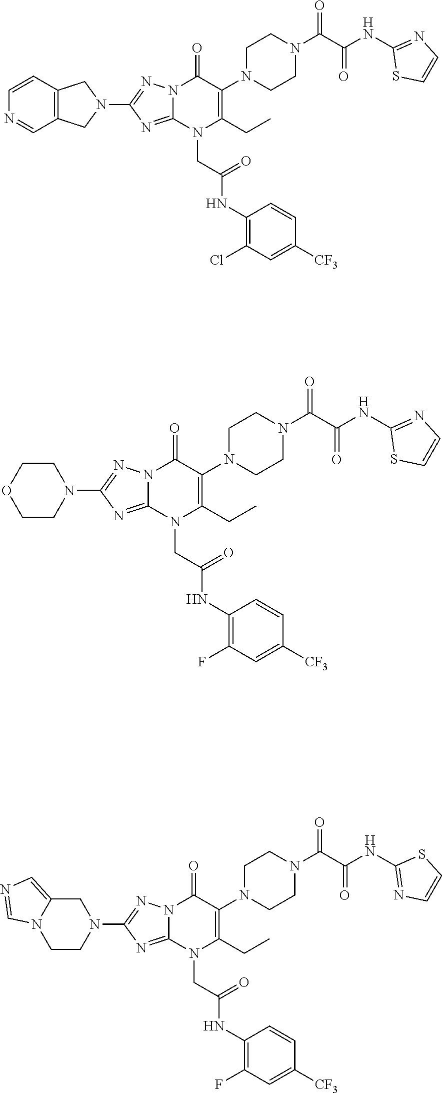 [SMILES: CCc1c(N2CCN(C(=O)C(=O)Nc3nccs3)CC2)c(=O)n2nc(N3Cc4ccncc4C3)nc2n1CC(=O)Nc1ccc(C(F)(F)F)cc1Cl ; CCc1c(N2CCN(C(=O)C(=O)Nc3nccs3)CC2)c(=O)n2nc(N3CCOCC3)nc2n1CC(=O)Nc1ccc(C(F)(F)F)cc1F ; CCc1c(N2CCN(C(=O)C(=O)Nc3nccs3)CC2)c(=O)n2nc(N3CCn4cncc4C3)nc2n1CC(=O)Nc1ccc(C(F)(F)F)cc1F]

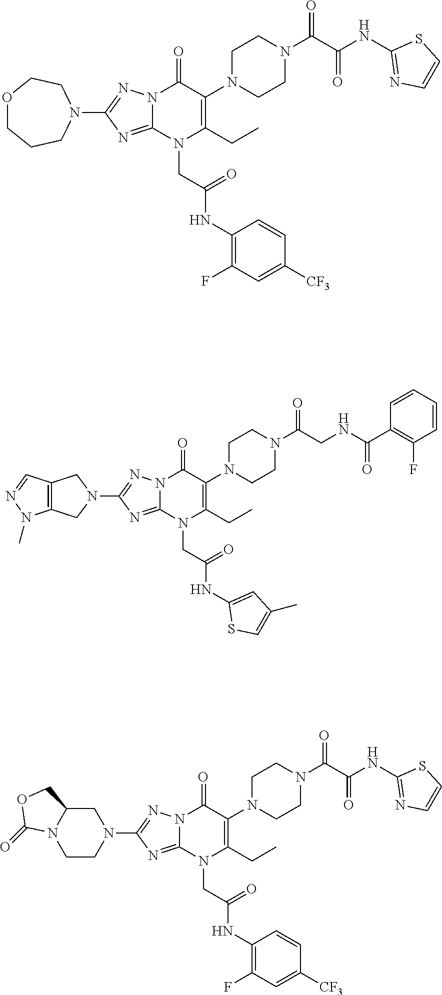 [SMILES: CCc1c(N2CCN(C(=O)C(=O)Nc3nccs3)CC2)c(=O)n2nc(N3CCCOCC3)nc2n1CC(=O)Nc1ccc(C(F)(F)F)cc1F ; CCc1c(N2CCN(C(=O)CNC(=O)c3ccccc3F)CC2)c(=O)n2nc(N3Cc4cnn(C)c4C3)nc2n1CC(=O)Nc1cc(C)cs1 ; CCc1c(N2CCN(C(=O)C(=O)Nc3nccs3)CC2)c(=O)n2nc(N3CCN4C(=O)OC[C@H]4C3)nc2n1CC(=O)Nc1ccc(C(F)(F)F)cc1F]

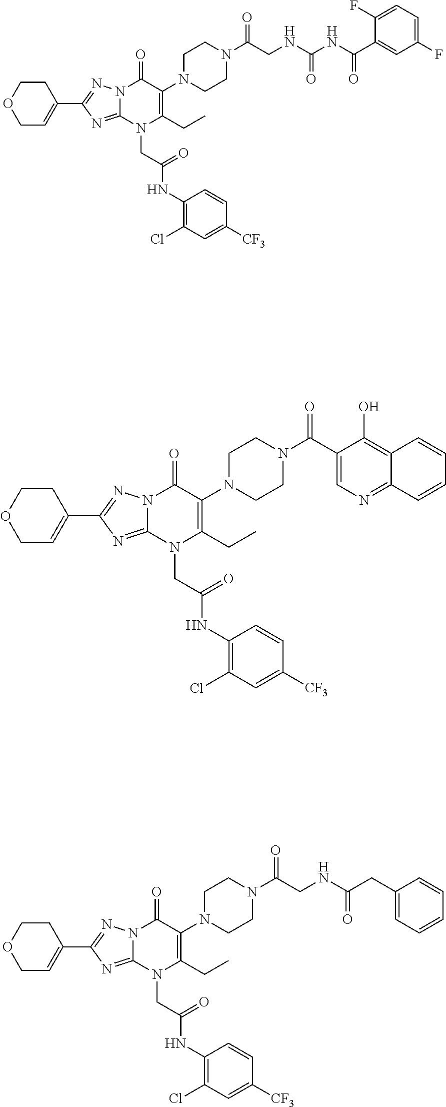 [SMILES: CCc1c(N2CCN(C(=O)CNC(=O)NC(=O)c3cc(F)ccc3F)CC2)c(=O)n2nc(C3=CCOCC3)nc2n1CC(=O)Nc1ccc(C(F)(F)F)cc1Cl ; CCc1c(N2CCN(C(=O)c3cnc4ccccc4c3O)CC2)c(=O)n2nc(C3=CCOCC3)nc2n1CC(=O)Nc1ccc(C(F)(F)F)cc1Cl ; CCc1c(N2CCN(C(=O)CNC(=O)Cc3ccccc3)CC2)c(=O)n2nc(C3=CCOCC3)nc2n1CC(=O)Nc1ccc(C(F)(F)F)cc1Cl]

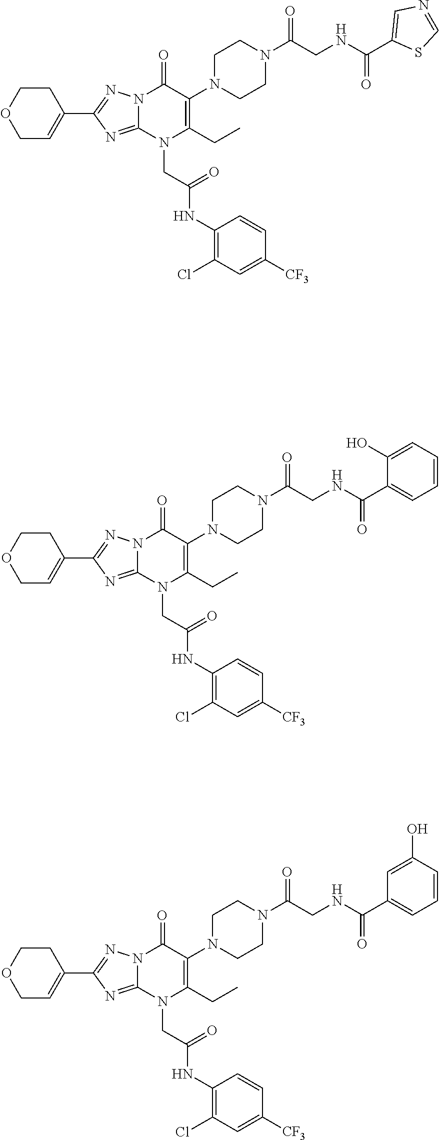 [SMILES: CCc1c(N2CCN(C(=O)CNC(=O)c3cncs3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(O)c3)CC2)c(=O)n2nc(C3=CCOCC3)nc2n1CC(=O)Nc1ccc(C(F)(F)F)cc1Cl]

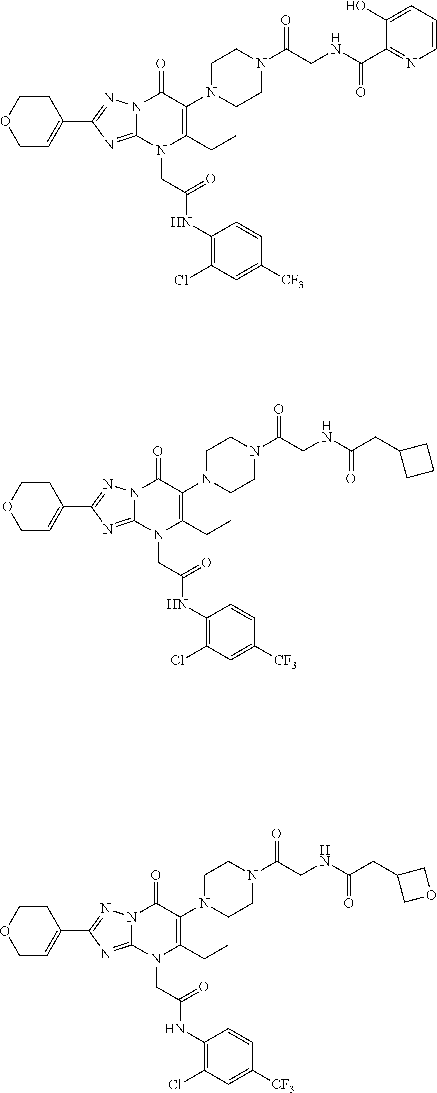 [SMILES: CCc1c(N2CCN(C(=O)CNC(=O)c3ncccc3O)CC2)c(=O)n2nc(C3=CCOCC3)nc2n1CC(=O)Nc1ccc(C(F)(F)F)cc1Cl ; CCc1c(N2CCN(C(=O)CNC(=O)CC3CCC3)CC2)c(=O)n2nc(C3=CCOCC3)nc2n1CC(=O)Nc1ccc(C(F)(F)F)cc1Cl ; CCc1c(N2CCN(C(=O)CNC(=O)CC3COC3)CC2)c(=O)n2nc(C3=CCOCC3)nc2n1CC(=O)Nc1ccc(C(F)(F)F)cc1Cl]

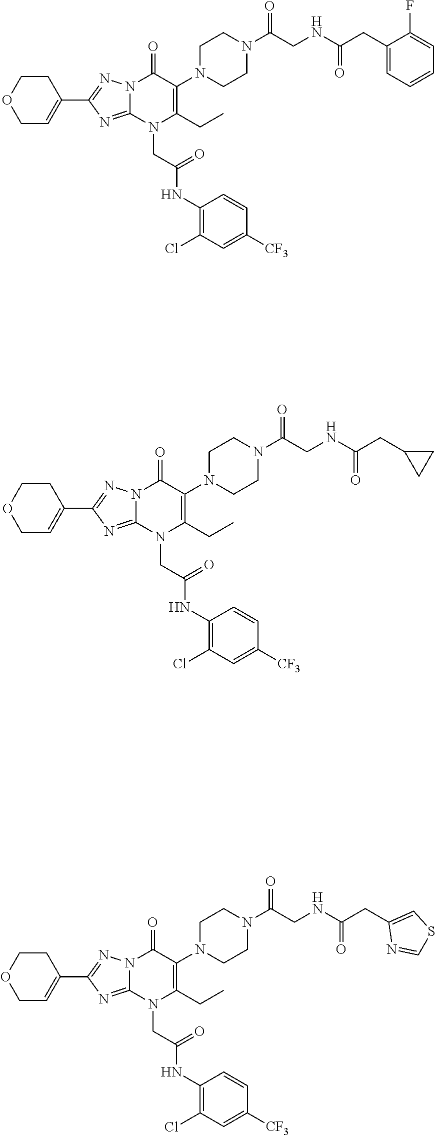 [SMILES: CCc1c(N2CCN(C(=O)CNC(=O)Cc3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)CC3CC3)CC2)c(=O)n2nc(C3=CCOCC3)nc2n1CC(=O)Nc1ccc(C(F)(F)F)cc1Cl ; CCc1c(N2CCN(C(=O)CNC(=O)Cc3cscn3)CC2)c(=O)n2nc(C3=CCOCC3)nc2n1CC(=O)Nc1ccc(C(F)(F)F)cc1Cl]

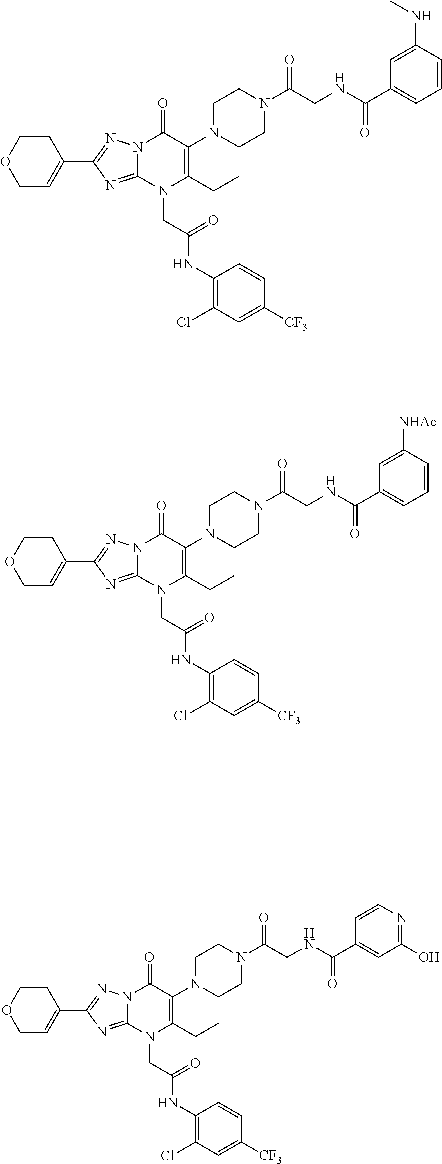 [SMILES: CCc1c(N2CCN(C(=O)CNC(=O)c3cccc(NC)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC(C)=O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccnc(O)c3)CC2)c(=O)n2nc(C3=CCOCC3)nc2n1CC(=O)Nc1ccc(C(F)(F)F)cc1Cl]

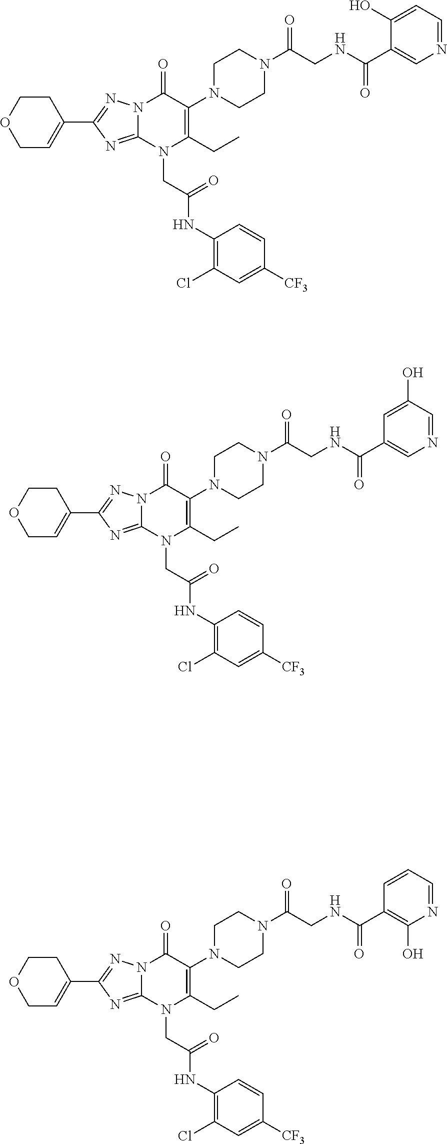 [SMILES: CCc1c(N2CCN(C(=O)CNC(=O)c3cnccc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cncc(O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccnc3O)CC2)c(=O)n2nc(C3=CCOCC3)nc2n1CC(=O)Nc1ccc(C(F)(F)F)cc1Cl]

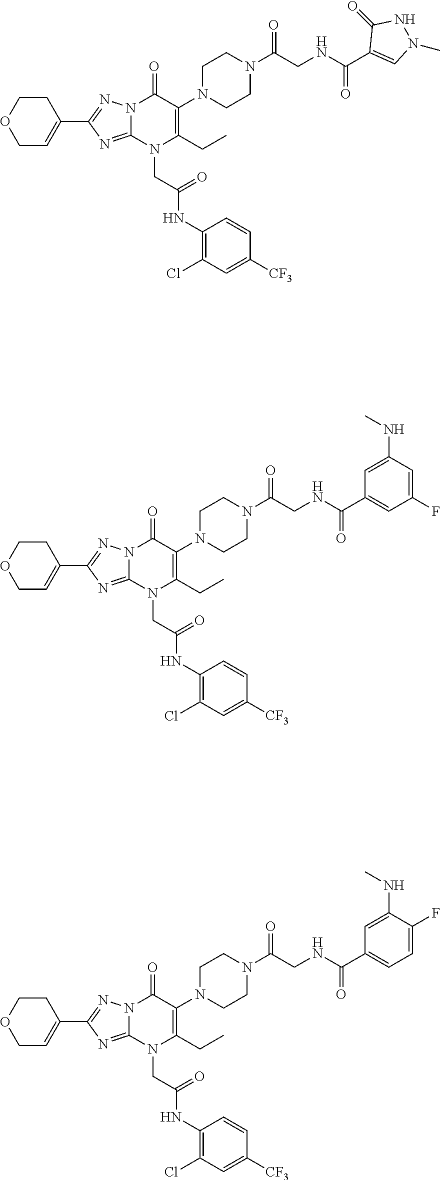 [SMILES: CCc1c(N2CCN(C(=O)CNC(=O)c3cn(C)[nH]c3=O)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(F)cc(NC)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)c(NC)c3)CC2)c(=O)n2nc(C3=CCOCC3)nc2n1CC(=O)Nc1ccc(C(F)(F)F)cc1Cl]

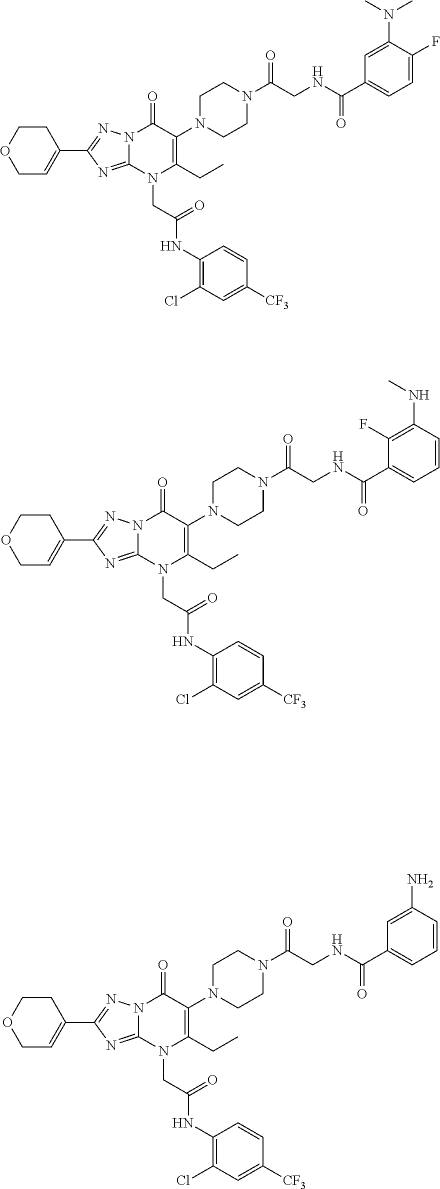 [SMILES: CCc1c(N2CCN(C(=O)CNC(=O)c3ccc(F)c(N(C)C)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC)c3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(N)c3)CC2)c(=O)n2nc(C3=CCOCC3)nc2n1CC(=O)Nc1ccc(C(F)(F)F)cc1Cl]

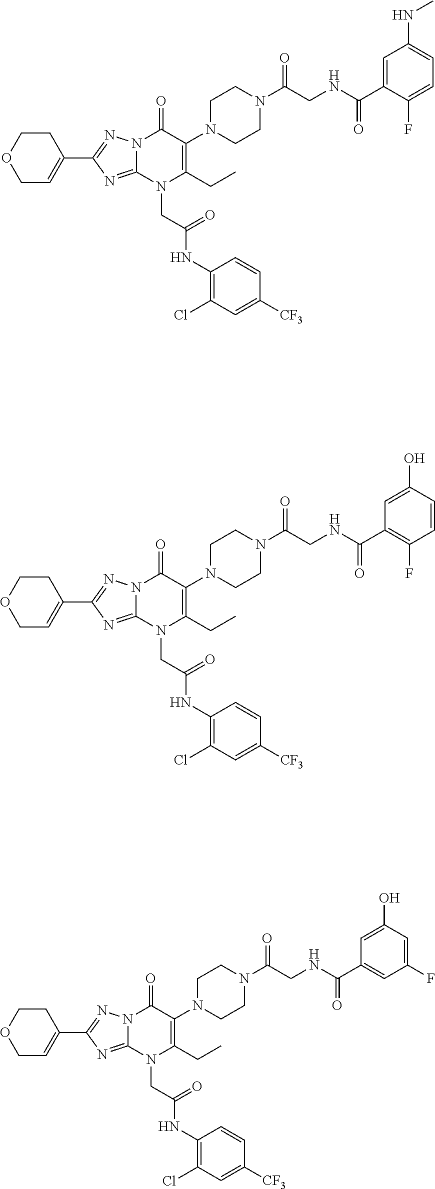 [SMILES: CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(O)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(O)cc(F)c3)CC2)c(=O)n2nc(C3=CCOCC3)nc2n1CC(=O)Nc1ccc(C(F)(F)F)cc1Cl]

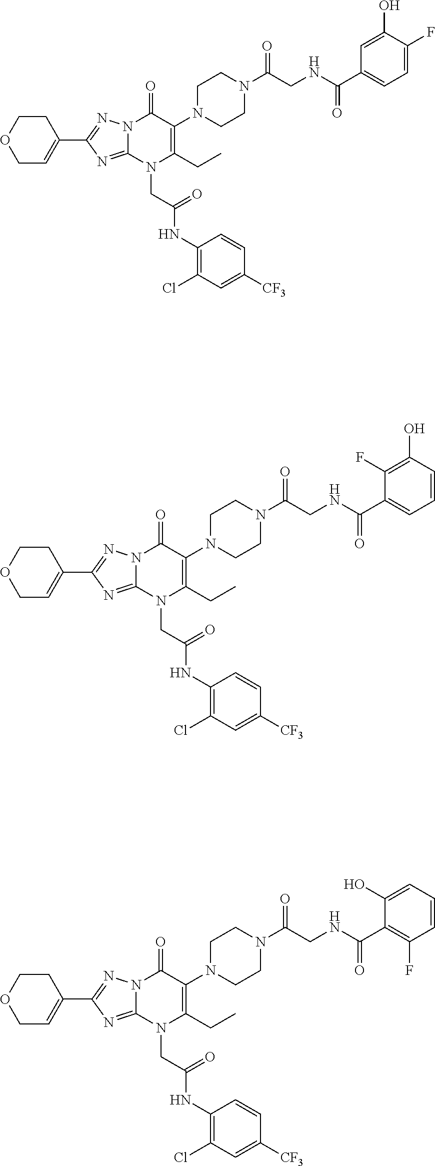 [SMILES: CCc1c(N2CCN(C(=O)CNC(=O)c3ccc(F)c(O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(O)c3F)CC2)c(=O)n2nc(C3=CCOCC3)nc2n1CC(=O)Nc1ccc(C(F)(F)F)cc1Cl ; CCc1c(N2CCN(C(=O)CNC(=O)c3c(O)cccc3F)CC2)c(=O)n2nc(C3=CCOCC3)nc2n1CC(=O)Nc1ccc(C(F)(F)F)cc1Cl]

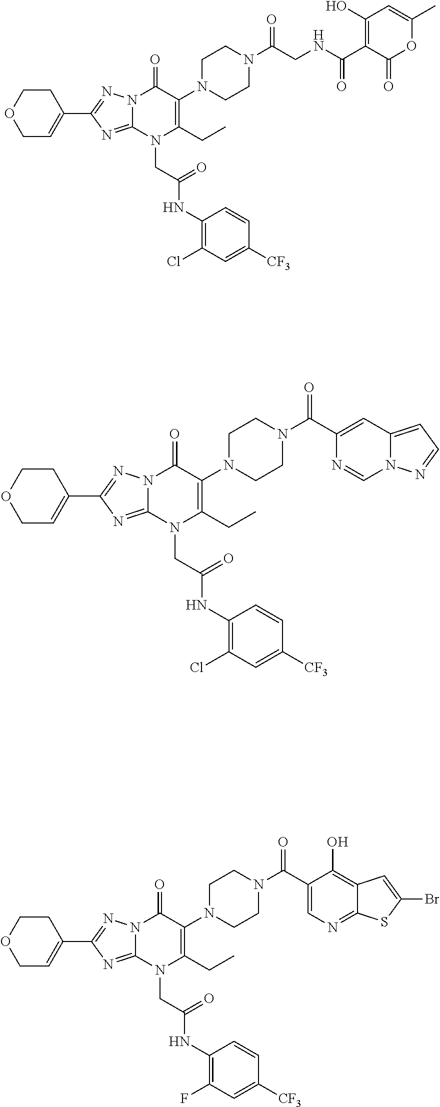 [SMILES: CCc1c(N2CCN(C(=O)CNC(=O)c3c(O)cc(C)oc3=O)CC2)c(=O)n2nc(C3=CCOCC3)nc2n1CC(=O)Nc1ccc(C(F)(F)F)cc1Cl ; CCc1c(N2CCN(C(=O)c3cc4ccnn4cn3)CC2)c(=O)n2nc(C3=CCOCC3)nc2n1CC(=O)Nc1ccc(C(F)(F)F)cc1Cl ; CCc1c(N2CCN(C(=O)c3cnc4sc(Br)cc4c3O)CC2)c(=O)n2nc(C3=CCOCC3)nc2n1CC(=O)Nc1ccc(C(F)(F)F)cc1F]

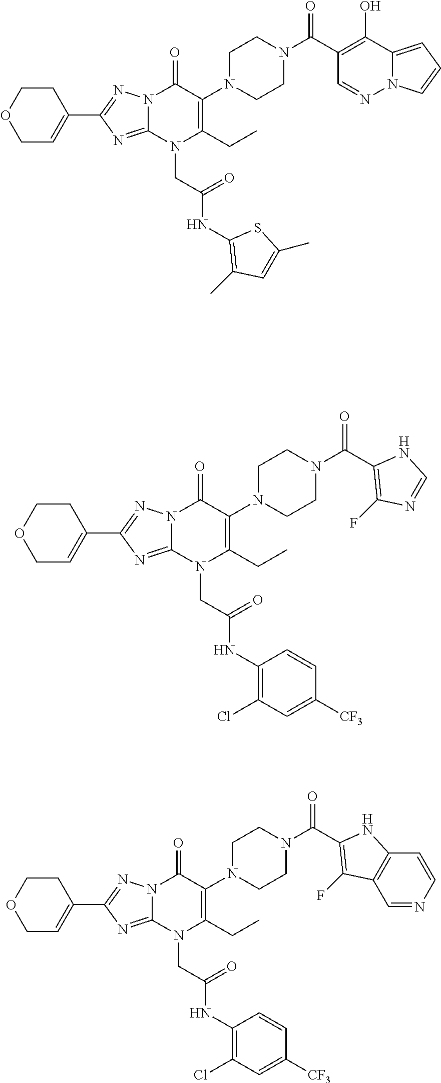 [SMILES: CCc1c(N2CCN(C(=O)c3cnn4cccc4c3O)CC2)c(=O)n2nc(C3=CCOCC3)nc2n1CC(=O)Nc1sc(C)cc1C ; CCc1c(N2CCN(C(=O)c3[nH]cnc3F)CC2)c(=O)n2nc(C3=CCOCC3)nc2n1CC(=O)Nc1ccc(C(F)(F)F)cc1Cl ; CCc1c(N2CCN(C(=O)c3[nH]c4ccncc4c3F)CC2)c(=O)n2nc(C3=CCOCC3)nc2n1CC(=O)Nc1ccc(C(F)(F)F)cc1Cl]

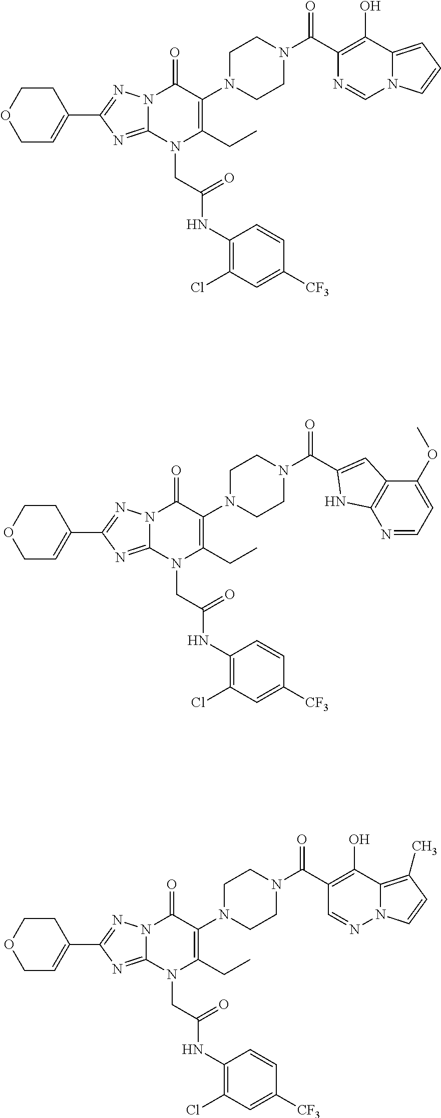 [SMILES: CCc1c(N2CCN(C(=O)c3ncn4cccc4c3O)CC2)c(=O)n2nc(C3=CCOCC3)nc2n1CC(=O)Nc1ccc(C(F)(F)F)cc1Cl ; CCc1c(N2CCN(C(=O)c3cc4c(OC)ccnc4[nH]3)CC2)c(=O)n2nc(C3=CCOCC3)nc2n1CC(=O)Nc1ccc(C(F)(F)F)cc1Cl ; CCc1c(N2CCN(C(=O)c3cnn4ccc(C)c4c3O)CC2)c(=O)n2nc(C3=CCOCC3)nc2n1CC(=O)Nc1ccc(C(F)(F)F)cc1Cl]

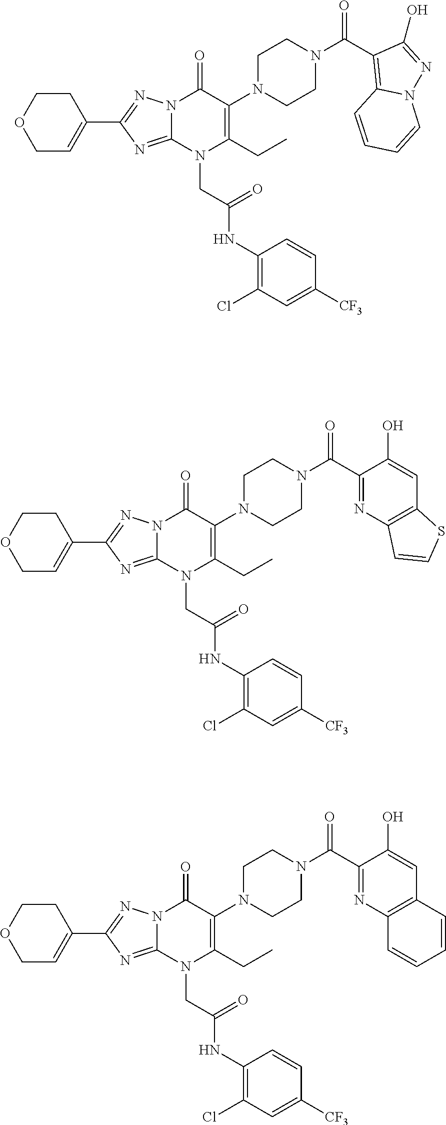 [SMILES: CCc1c(N2CCN(C(=O)c3c(O)nn4ccccc34)CC2)c(=O)n2nc(C3=CCOCC3)nc2n1CC(=O)Nc1ccc(C(F)(F)F)cc1Cl ; CCc1c(N2CCN(C(=O)c3nc4ccsc4cc3O)CC2)c(=O)n2nc(C3=CCOCC3)nc2n1CC(=O)Nc1ccc(C(F)(F)F)cc1Cl ; CCc1c(N2CCN(C(=O)c3nc4ccccc4cc3O)CC2)c(=O)n2nc(C3=CCOCC3)nc2n1CC(=O)Nc1ccc(C(F)(F)F)cc1Cl]

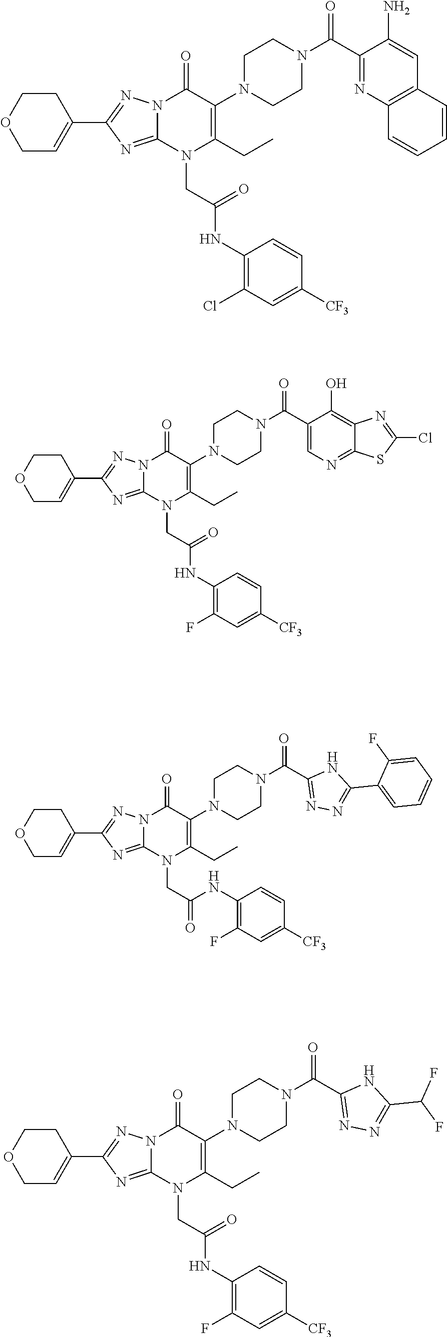 [SMILES: CCc1c(N2CCN(C(=O)c3nc4ccccc4cc3N)CC2)c(=O)n2nc(C3=CCOCC3)nc2n1CC(=O)Nc1ccc(C(F)(F)F)cc1Cl ; CCc1c(N2CCN(C(=O)c3cnc4sc(Cl)nc4c3O)CC2)c(=O)n2nc(C3=CCOCC3)nc2n1CC(=O)Nc1ccc(C(F)(F)F)cc1F ; CCc1c(N2CCN(C(=O)c3nnc(-c4ccccc4F)[nH]3)CC2)c(=O)n2nc(C3=CCOCC3)nc2n1CC(=O)Nc1ccc(C(F)(F)F)cc1F ; CCc1c(N2CCN(C(=O)c3nnc(C(F)F)[nH]3)CC2)c(=O)n2nc(C3=CCOCC3)nc2n1CC(=O)Nc1ccc(C(F)(F)F)cc1F]

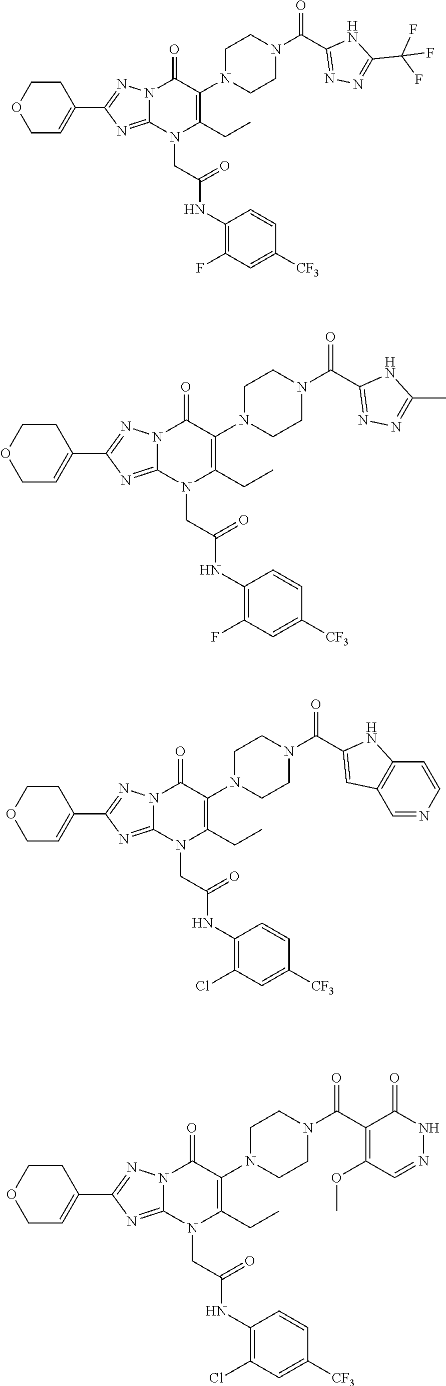 [SMILES: CCc1c(N2CCN(C(=O)c3nnc(C(F)(F)F)[nH]3)CC2)c(=O)n2nc(C3=CCOCC3)nc2n1CC(=O)Nc1ccc(C(F)(F)F)cc1F ; CCc1c(N2CCN(C(=O)c3nnc(C)[nH]3)CC2)c(=O)n2nc(C3=CCOCC3)nc2n1CC(=O)Nc1ccc(C(F)(F)F)cc1F ; CCc1c(N2CCN(C(=O)c3cc4cnccc4[nH]3)CC2)c(=O)n2nc(C3=CCOCC3)nc2n1CC(=O)Nc1ccc(C(F)(F)F)cc1Cl ; CCc1c(N2CCN(C(=O)c3c(OC)cn[nH]c3=O)CC2)c(=O)n2nc(C3=CCOCC3)nc2n1CC(=O)Nc1ccc(C(F)(F)F)cc1Cl]

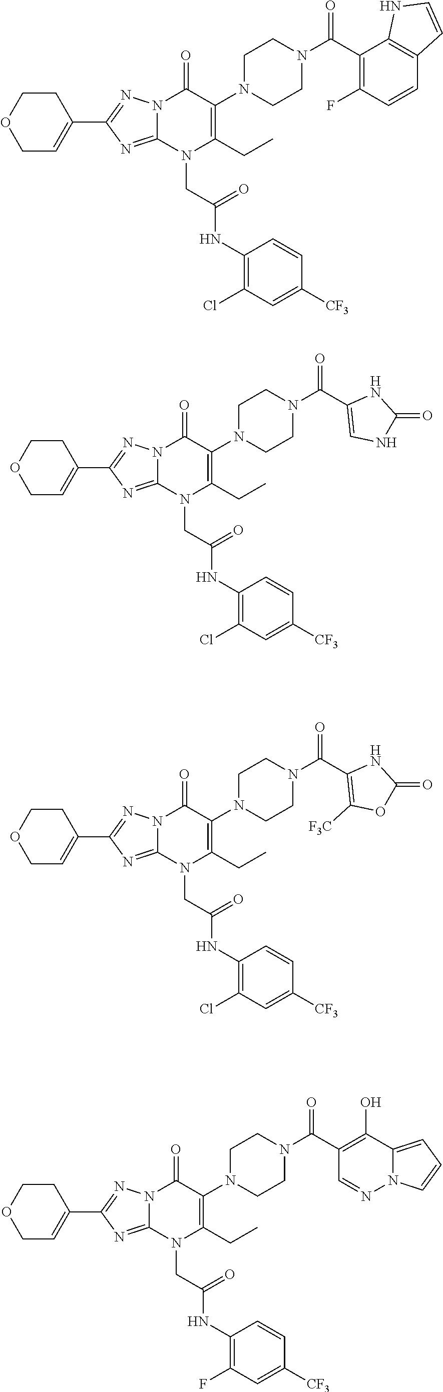 [SMILES: CCc1c(N2CCN(C(=O)c3c(F)ccc4cc[nH]c34)CC2)c(=O)n2nc(C3=CCOCC3)nc2n1CC(=O)Nc1ccc(C(F)(F)F)cc1Cl ; CCc1c(N2CCN(C(=O)c3c[nH]c(=O)[nH]3)CC2)c(=O)n2nc(C3=CCOCC3)nc2n1CC(=O)Nc1ccc(C(F)(F)F)cc1Cl ; CCc1c(N2CCN(C(=O)c3[nH]c(=O)oc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3cnn4cccc4c3O)CC2)c(=O)n2nc(C3=CCOCC3)nc2n1CC(=O)Nc1ccc(C(F)(F)F)cc1F]

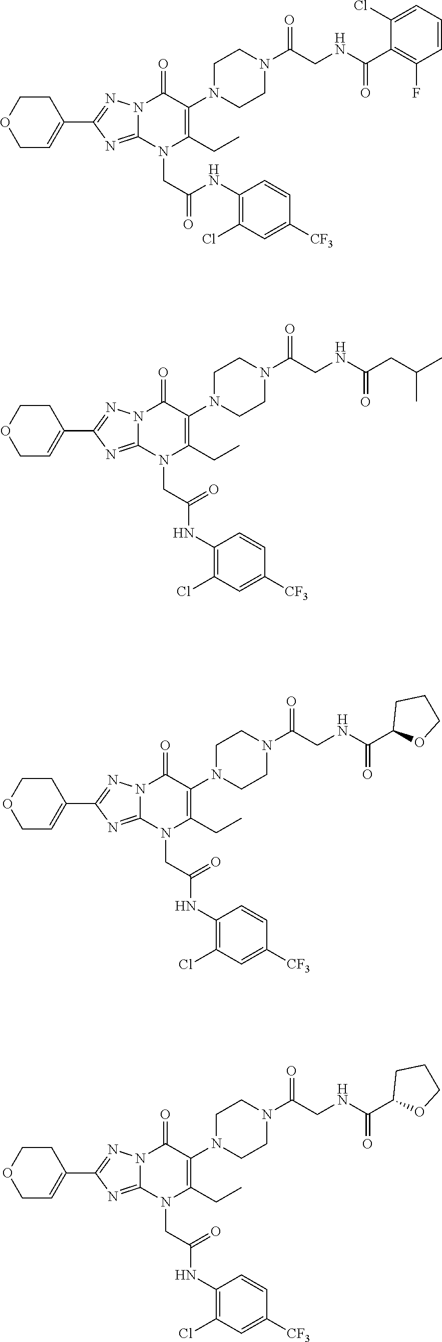 [SMILES: CCc1c(N2CCN(C(=O)CNC(=O)c3c(F)cccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)CC(C)C)CC2)c(=O)n2nc(C3=CCOCC3)nc2n1CC(=O)Nc1ccc(C(F)(F)F)cc1Cl ; CCc1c(N2CCN(C(=O)CNC(=O)[C@H]3CCCO3)CC2)c(=O)n2nc(C3=CCOCC3)nc2n1CC(=O)Nc1ccc(C(F)(F)F)cc1Cl ; CCc1c(N2CCN(C(=O)CNC(=O)[C@@H]3CCCO3)CC2)c(=O)n2nc(C3=CCOCC3)nc2n1CC(=O)Nc1ccc(C(F)(F)F)cc1Cl]

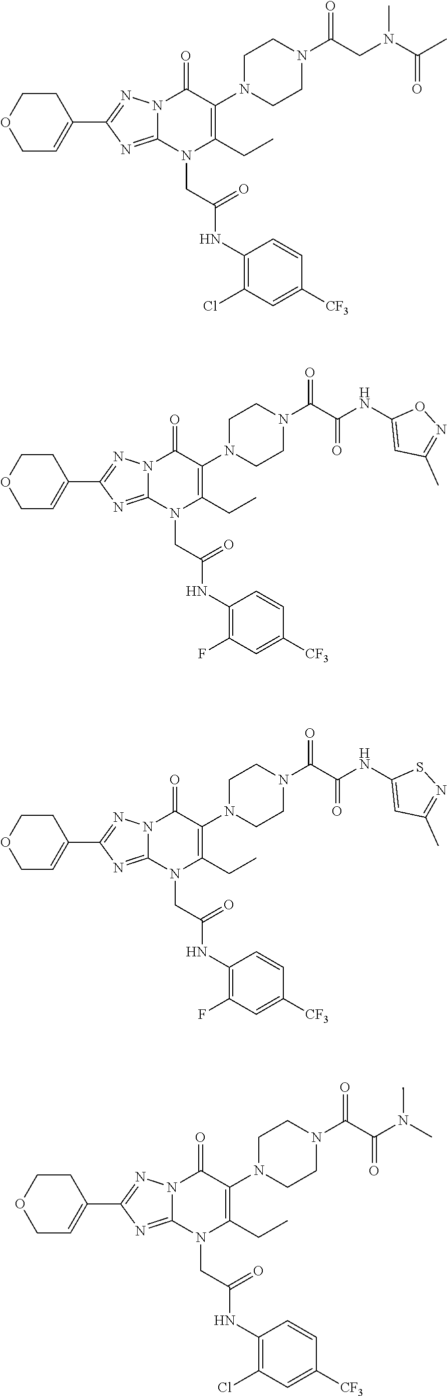 [SMILES: CCc1c(N2CCN(C(=O)CN(C)C(C)=O)CC2)c(=O)n2nc(C3=CCOCC3)nc2n1CC(=O)Nc1ccc(C(F)(F)F)cc1Cl ; CCc1c(N2CCN(C(=O)C(=O)Nc3cc(C)no3)CC2)c(=O)n2nc(C3=CCOCC3)nc2n1CC(=O)Nc1ccc(C(F)(F)F)cc1F ; CCc1c(N2CCN(C(=O)C(=O)Nc3cc(C)ns3)CC2)c(=O)n2nc(C3=CCOCC3)nc2n1CC(=O)Nc1ccc(C(F)(F)F)cc1F ; CCc1c(N2CCN(C(=O)C(=O)N(C)C)CC2)c(=O)n2nc(C3=CCOCC3)nc2n1CC(=O)Nc1ccc(C(F)(F)F)cc1Cl]

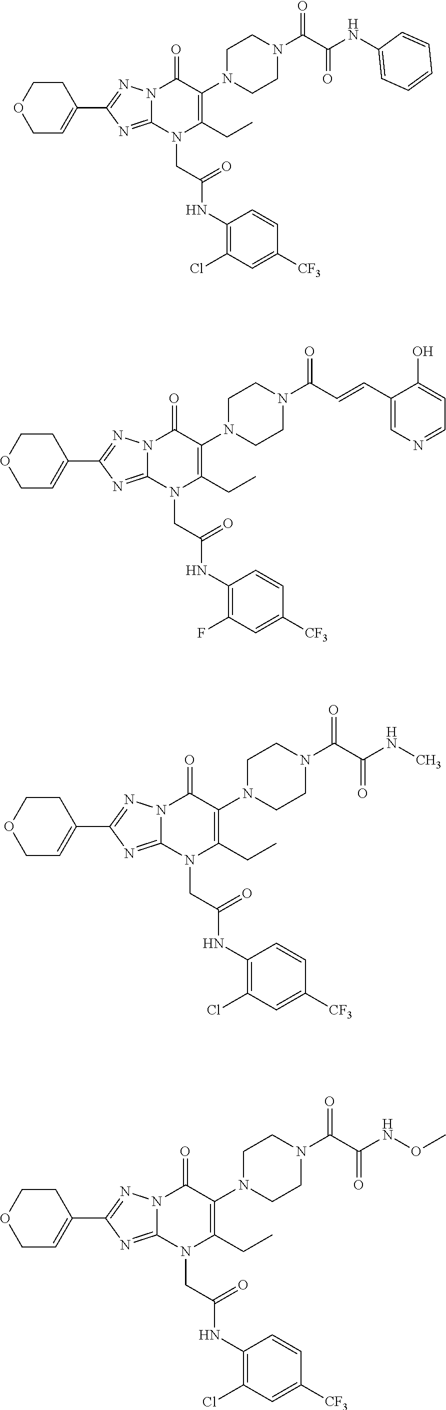 [SMILES: CCc1c(N2CCN(C(=O)C(=O)Nc3ccccc3)CC2)c(=O)n2nc(C3=CCOCC3)nc2n1CC(=O)Nc1ccc(C(F)(F)F)cc1Cl ; CCc1c(N2CCN(C(=O)/C=C/c3cnccc3O)CC2)c(=O)n2nc(C3=CCOCC3)nc2n1CC(=O)Nc1ccc(C(F)(F)F)cc1F ; CCc1c(N2CCN(C(=O)C(=O)NC)CC2)c(=O)n2nc(C3=CCOCC3)nc2n1CC(=O)Nc1ccc(C(F)(F)F)cc1Cl ; CCc1c(N2CCN(C(=O)C(=O)NOC)CC2)c(=O)n2nc(C3=CCOCC3)nc2n1CC(=O)Nc1ccc(C(F)(F)F)cc1Cl]

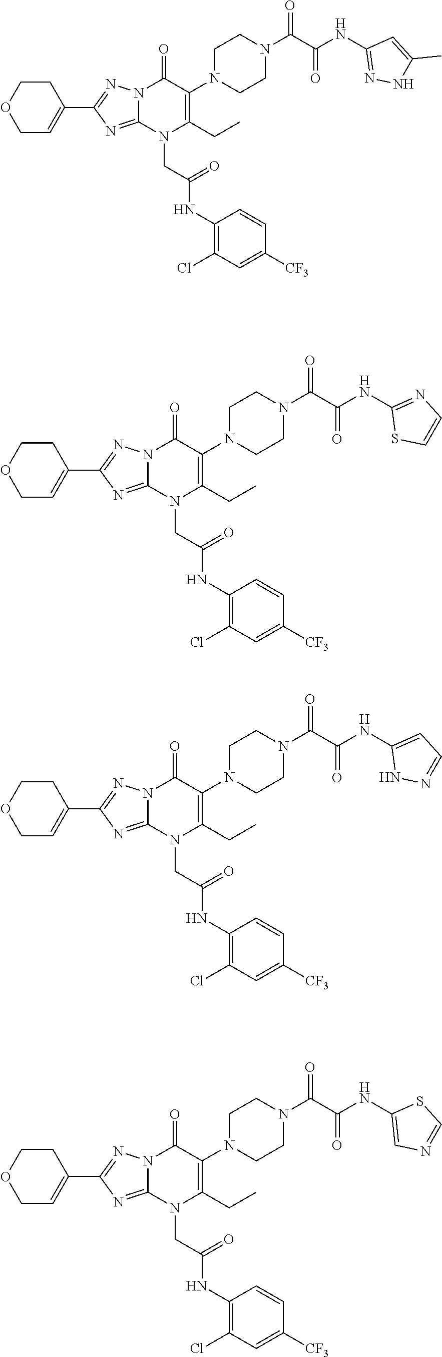 [SMILES: CCc1c(N2CCN(C(=O)C(=O)Nc3cc(C)[nH]n3)CC2)c(=O)n2nc(C3=CCOCC3)nc2n1CC(=O)Nc1ccc(C(F)(F)F)cc1Cl ; CCc1c(N2CCN(C(=O)C(=O)Nc3nccs3)CC2)c(=O)n2nc(C3=CCOCC3)nc2n1CC(=O)Nc1ccc(C(F)(F)F)cc1Cl ; CCc1c(N2CCN(C(=O)C(=O)Nc3ccn[nH]3)CC2)c(=O)n2nc(C3=CCOCC3)nc2n1CC(=O)Nc1ccc(C(F)(F)F)cc1Cl ; CCc1c(N2CCN(C(=O)C(=O)Nc3cncs3)CC2)c(=O)n2nc(C3=CCOCC3)nc2n1CC(=O)Nc1ccc(C(F)(F)F)cc1Cl]

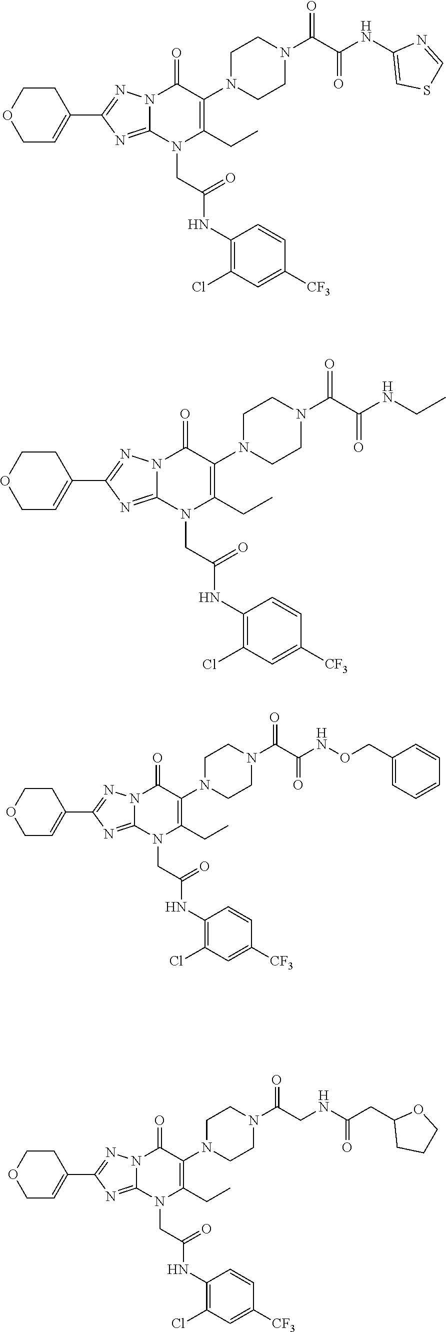 [SMILES: CCc1c(N2CCN(C(=O)C(=O)Nc3cscn3)CC2)c(=O)n2nc(C3=CCOCC3)nc2n1CC(=O)Nc1ccc(C(F)(F)F)cc1Cl ; CCNC(=O)C(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)C(=O)NOCc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)CC3CCCO3)CC2)c(=O)n2nc(C3=CCOCC3)nc2n1CC(=O)Nc1ccc(C(F)(F)F)cc1Cl]

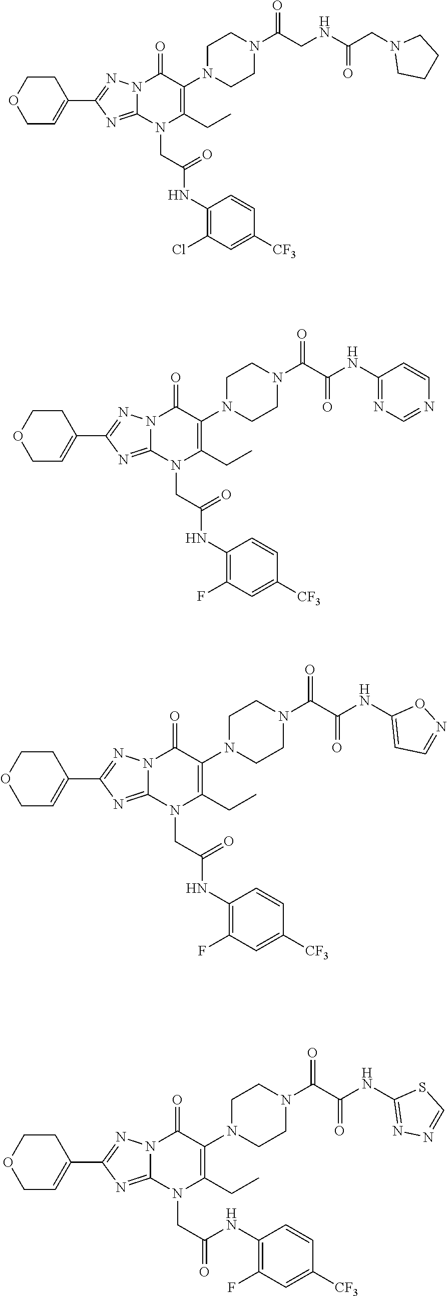 [SMILES: CCc1c(N2CCN(C(=O)CNC(=O)CN3CCCC3)CC2)c(=O)n2nc(C3=CCOCC3)nc2n1CC(=O)Nc1ccc(C(F)(F)F)cc1Cl ; CCc1c(N2CCN(C(=O)C(=O)Nc3ccncn3)CC2)c(=O)n2nc(C3=CCOCC3)nc2n1CC(=O)Nc1ccc(C(F)(F)F)cc1F ; CCc1c(N2CCN(C(=O)C(=O)Nc3ccno3)CC2)c(=O)n2nc(C3=CCOCC3)nc2n1CC(=O)Nc1ccc(C(F)(F)F)cc1F ; CCc1c(N2CCN(C(=O)C(=O)Nc3nncs3)CC2)c(=O)n2nc(C3=CCOCC3)nc2n1CC(=O)Nc1ccc(C(F)(F)F)cc1F]

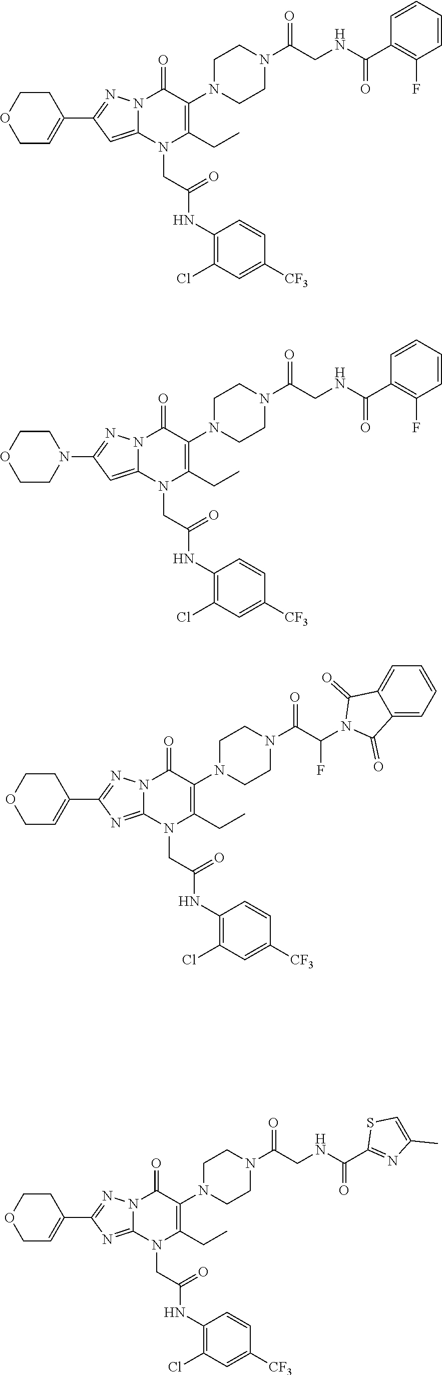 [SMILES: CCc1c(N2CCN(C(=O)CNC(=O)c3ccccc3F)CC2)c(=O)n2nc(C3=CCOCC3)cc2n1CC(=O)Nc1ccc(C(F)(F)F)cc1Cl ; CCc1c(N2CCN(C(=O)CNC(=O)c3ccccc3F)CC2)c(=O)n2nc(N3CCOCC3)cc2n1CC(=O)Nc1ccc(C(F)(F)F)cc1Cl ; CCc1c(N2CCN(C(=O)C(F)N3C(=O)c4ccccc4C3=O)CC2)c(=O)n2nc(C3=CCOCC3)nc2n1CC(=O)Nc1ccc(C(F)(F)F)cc1Cl ; CCc1c(N2CCN(C(=O)CNC(=O)c3nc(C)cs3)CC2)c(=O)n2nc(C3=CCOCC3)nc2n1CC(=O)Nc1ccc(C(F)(F)F)cc1Cl]

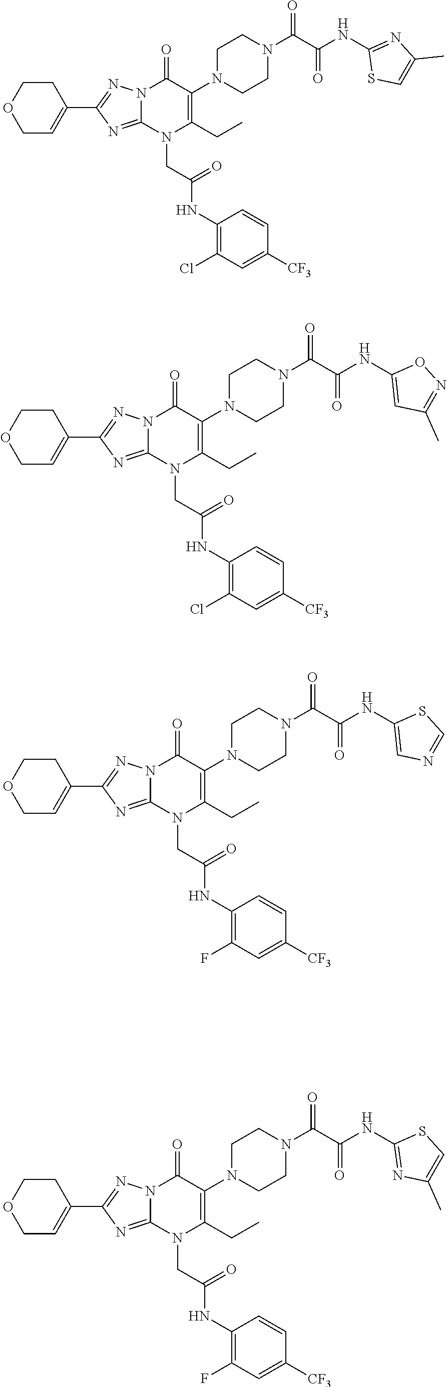 [SMILES: CCc1c(N2CCN(C(=O)C(=O)Nc3nc(C)cs3)CC2)c(=O)n2nc(C3=CCOCC3)nc2n1CC(=O)Nc1ccc(C(F)(F)F)cc1Cl ; CCc1c(N2CCN(C(=O)C(=O)Nc3cc(C)no3)CC2)c(=O)n2nc(C3=CCOCC3)nc2n1CC(=O)Nc1ccc(C(F)(F)F)cc1Cl ; CCc1c(N2CCN(C(=O)C(=O)Nc3cncs3)CC2)c(=O)n2nc(C3=CCOCC3)nc2n1CC(=O)Nc1ccc(C(F)(F)F)cc1F ; CCc1c(N2CCN(C(=O)C(=O)Nc3nc(C)cs3)CC2)c(=O)n2nc(C3=CCOCC3)nc2n1CC(=O)Nc1ccc(C(F)(F)F)cc1F]

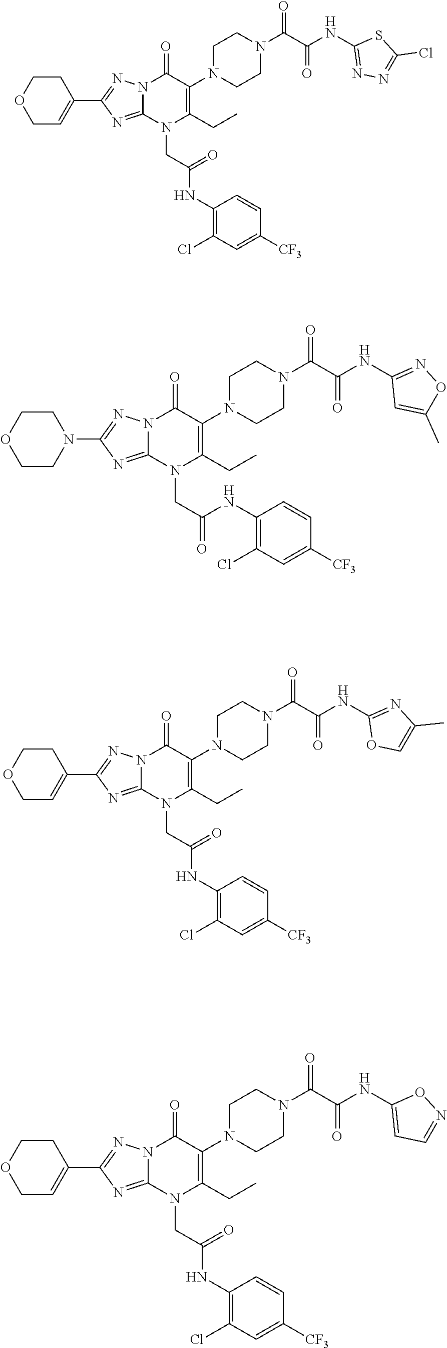 [SMILES: CCc1c(N2CCN(C(=O)C(=O)Nc3nnc(Cl)s3)CC2)c(=O)n2nc(C3=CCOCC3)nc2n1CC(=O)Nc1ccc(C(F)(F)F)cc1Cl ; CCc1c(N2CCN(C(=O)C(=O)Nc3cc(C)on3)CC2)c(=O)n2nc(N3CCOCC3)nc2n1CC(=O)Nc1ccc(C(F)(F)F)cc1Cl ; CCc1c(N2CCN(C(=O)C(=O)Nc3nc(C)co3)CC2)c(=O)n2nc(C3=CCOCC3)nc2n1CC(=O)Nc1ccc(C(F)(F)F)cc1Cl ; CCc1c(N2CCN(C(=O)C(=O)Nc3ccno3)CC2)c(=O)n2nc(C3=CCOCC3)nc2n1CC(=O)Nc1ccc(C(F)(F)F)cc1Cl]

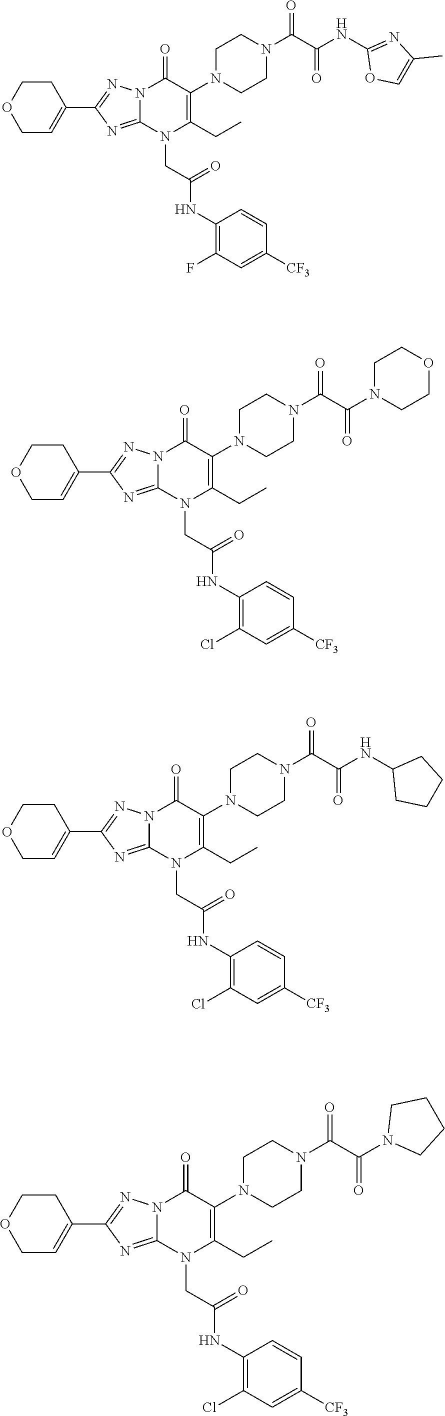 [SMILES: CCc1c(N2CCN(C(=O)C(=O)Nc3nc(C)co3)CC2)c(=O)n2nc(C3=CCOCC3)nc2n1CC(=O)Nc1ccc(C(F)(F)F)cc1F ; CCc1c(N2CCN(C(=O)C(=O)N3CCOCC3)CC2)c(=O)n2nc(C3=CCOCC3)nc2n1CC(=O)Nc1ccc(C(F)(F)F)cc1Cl ; CCc1c(N2CCN(C(=O)C(=O)NC3CCCC3)CC2)c(=O)n2nc(C3=CCOCC3)nc2n1CC(=O)Nc1ccc(C(F)(F)F)cc1Cl ; CCc1c(N2CCN(C(=O)C(=O)N3CCCC3)CC2)c(=O)n2nc(C3=CCOCC3)nc2n1CC(=O)Nc1ccc(C(F)(F)F)cc1Cl]

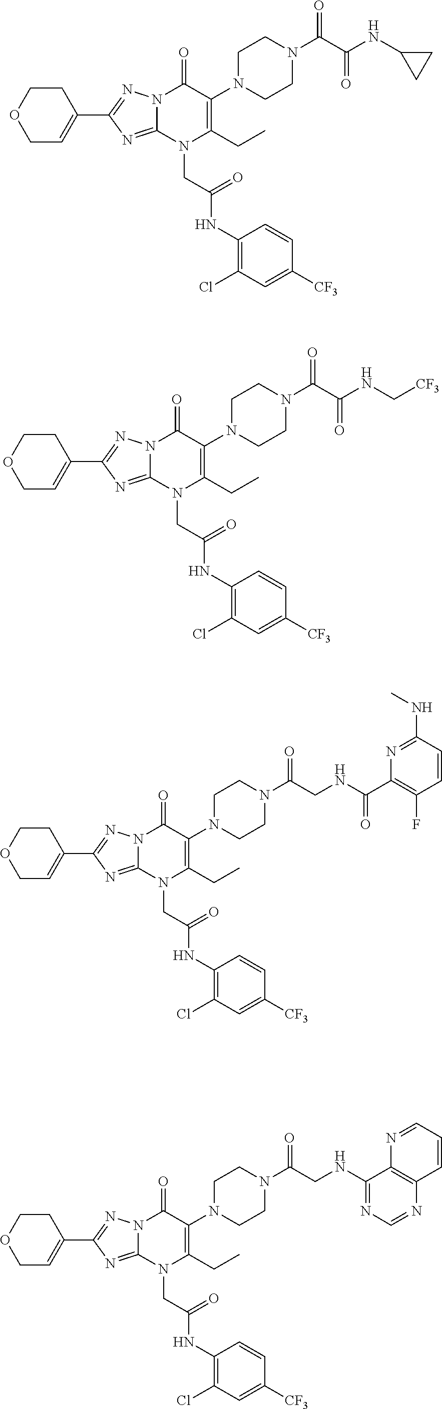 [SMILES: CCc1c(N2CCN(C(=O)C(=O)NC3CC3)CC2)c(=O)n2nc(C3=CCOCC3)nc2n1CC(=O)Nc1ccc(C(F)(F)F)cc1Cl ; CCc1c(N2CCN(C(=O)C(=O)NCC(F)(F)F)CC2)c(=O)n2nc(C3=CCOCC3)nc2n1CC(=O)Nc1ccc(C(F)(F)F)cc1Cl ; CCc1c(N2CCN(C(=O)CNC(=O)c3nc(NC)ccc3F)CC2)c(=O)n2nc(C3=CCOCC3)nc2n1CC(=O)Nc1ccc(C(F)(F)F)cc1Cl ; CCc1c(N2CCN(C(=O)CNc3ncnc4cccnc34)CC2)c(=O)n2nc(C3=CCOCC3)nc2n1CC(=O)Nc1ccc(C(F)(F)F)cc1Cl]

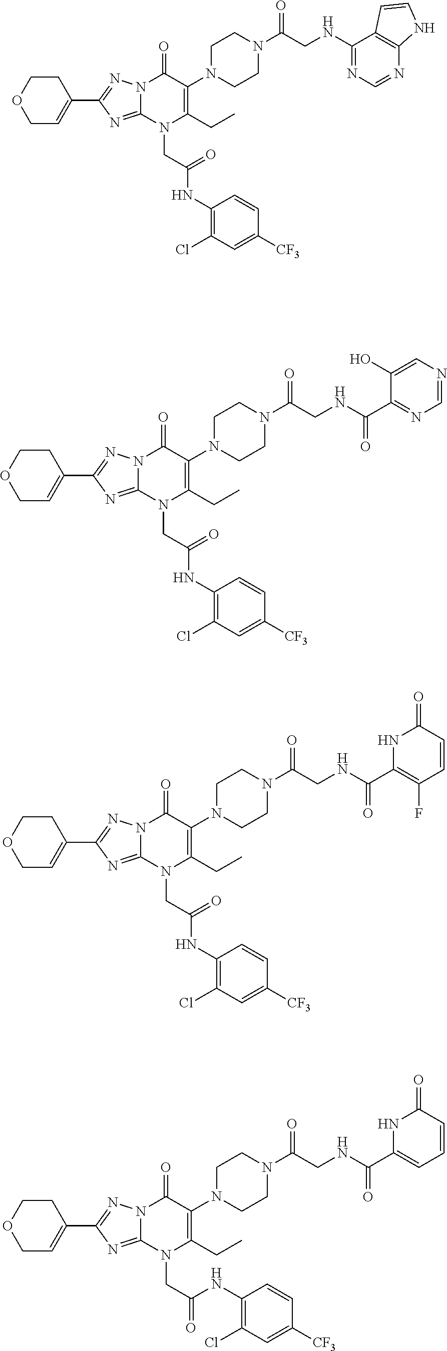 [SMILES: CCc1c(N2CCN(C(=O)CNc3ncnc4[nH]ccc34)CC2)c(=O)n2nc(C3=CCOCC3)nc2n1CC(=O)Nc1ccc(C(F)(F)F)cc1Cl ; CCc1c(N2CCN(C(=O)CNC(=O)c3ncncc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3[nH]c(=O)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(=O)[nH]3)CC2)c(=O)n2nc(C3=CCOCC3)nc2n1CC(=O)Nc1ccc(C(F)(F)F)cc1Cl]

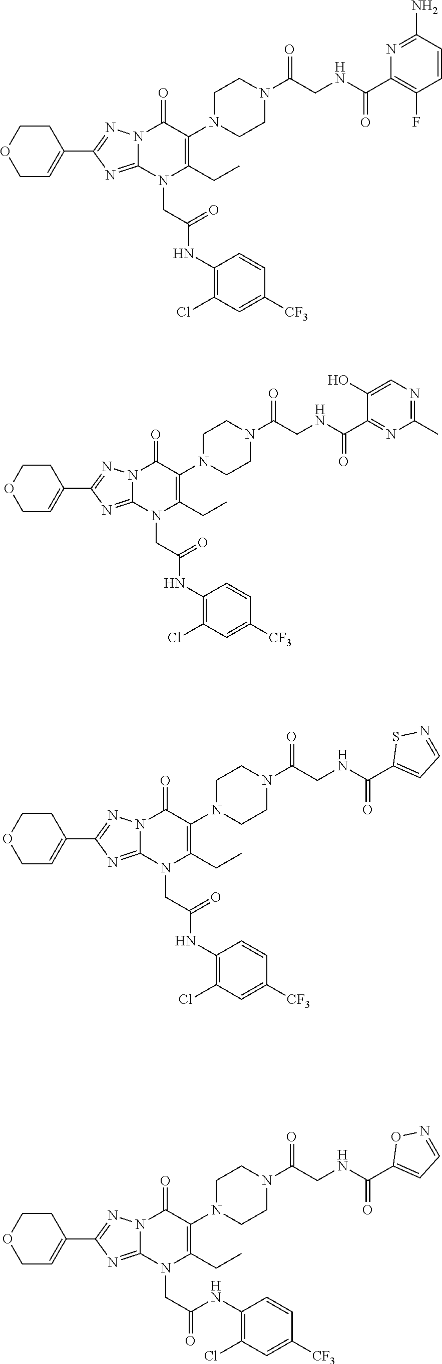 [SMILES: CCc1c(N2CCN(C(=O)CNC(=O)c3nc(N)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3nc(C)ncc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3ccns3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccno3)CC2)c(=O)n2nc(C3=CCOCC3)nc2n1CC(=O)Nc1ccc(C(F)(F)F)cc1Cl]

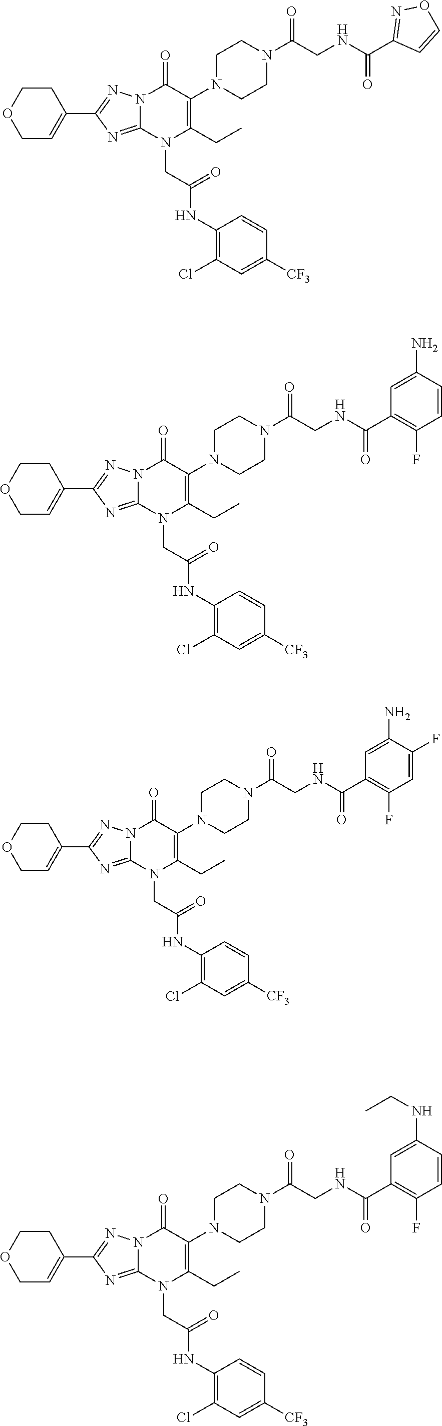 [SMILES: CCc1c(N2CCN(C(=O)CNC(=O)c3ccon3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(N)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(N)c(F)cc3F)CC2)c(=O)n2nc(C3=CCOCC3)nc2n1CC(=O)Nc1ccc(C(F)(F)F)cc1Cl ; CCNc1ccc(F)c(C(=O)NCC(=O)N2CCN(c3c(CC)n(CC(=O)Nc4ccc(C(F)(F)F)cc4Cl)c4nc(C5=CCOCC5)nn4c3=O)CC2)c1]

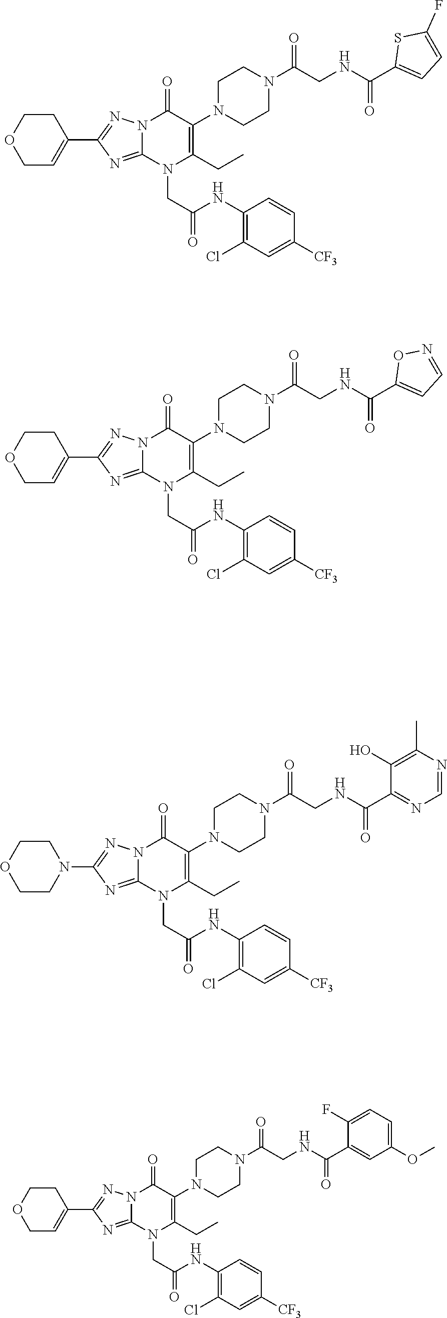 [SMILES: CCc1c(N2CCN(C(=O)CNC(=O)c3ccc(F)s3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccno3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncnc(C)c3O)CC2)c(=O)n2nc(N3CCOCC3)nc2n1CC(=O)Nc1ccc(C(F)(F)F)cc1Cl ; CCc1c(N2CCN(C(=O)CNC(=O)c3cc(OC)ccc3F)CC2)c(=O)n2nc(C3=CCOCC3)nc2n1CC(=O)Nc1ccc(C(F)(F)F)cc1Cl]

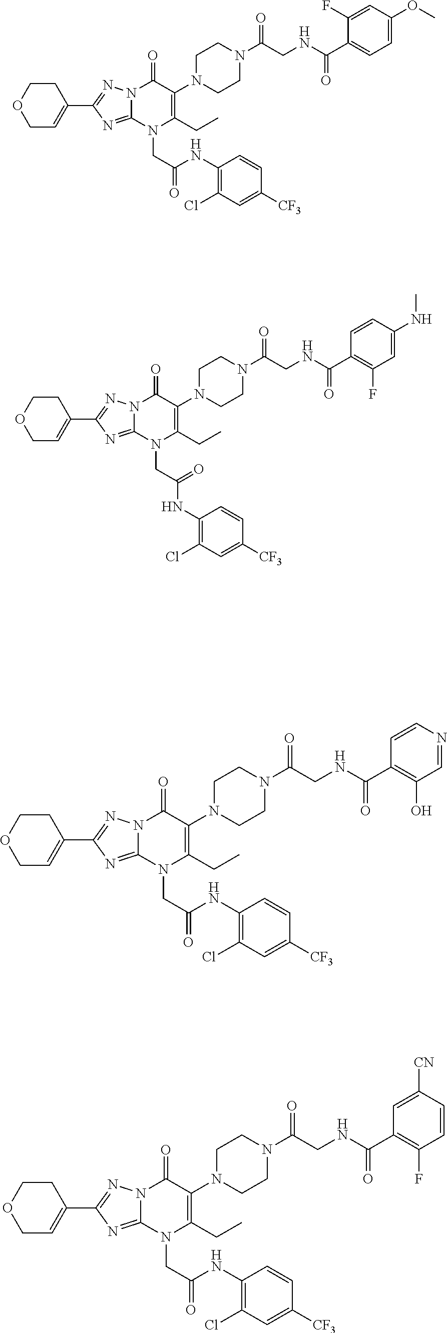 [SMILES: CCc1c(N2CCN(C(=O)CNC(=O)c3ccc(OC)cc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(NC)cc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccncc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C#N)ccc3F)CC2)c(=O)n2nc(C3=CCOCC3)nc2n1CC(=O)Nc1ccc(C(F)(F)F)cc1Cl]

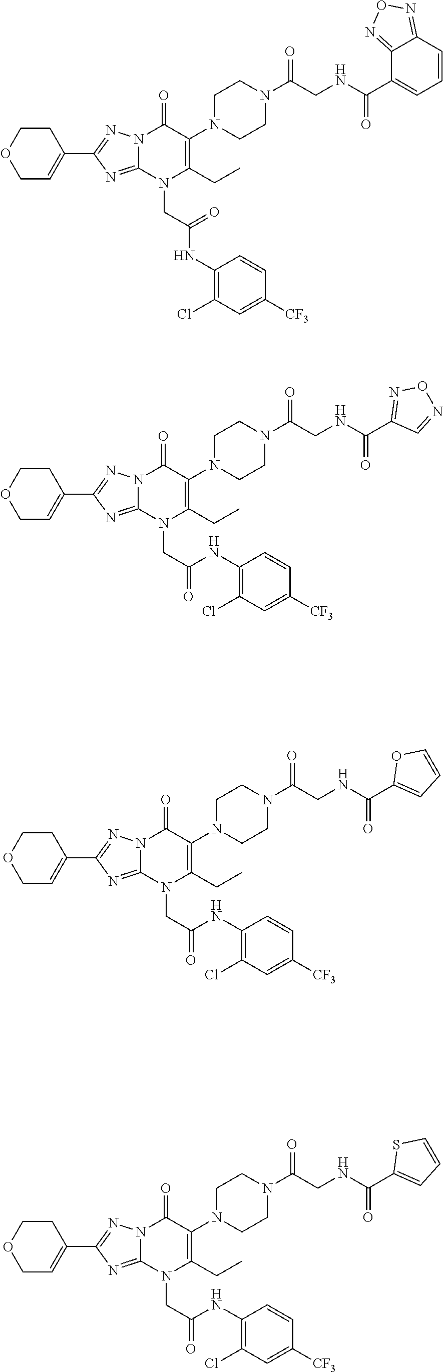 [SMILES: CCc1c(N2CCN(C(=O)CNC(=O)c3cccc4nonc34)CC2)c(=O)n2nc(C3=CCOCC3)nc2n1CC(=O)Nc1ccc(C(F)(F)F)cc1Cl ; CCc1c(N2CCN(C(=O)CNC(=O)c3cnon3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccco3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccs3)CC2)c(=O)n2nc(C3=CCOCC3)nc2n1CC(=O)Nc1ccc(C(F)(F)F)cc1Cl]

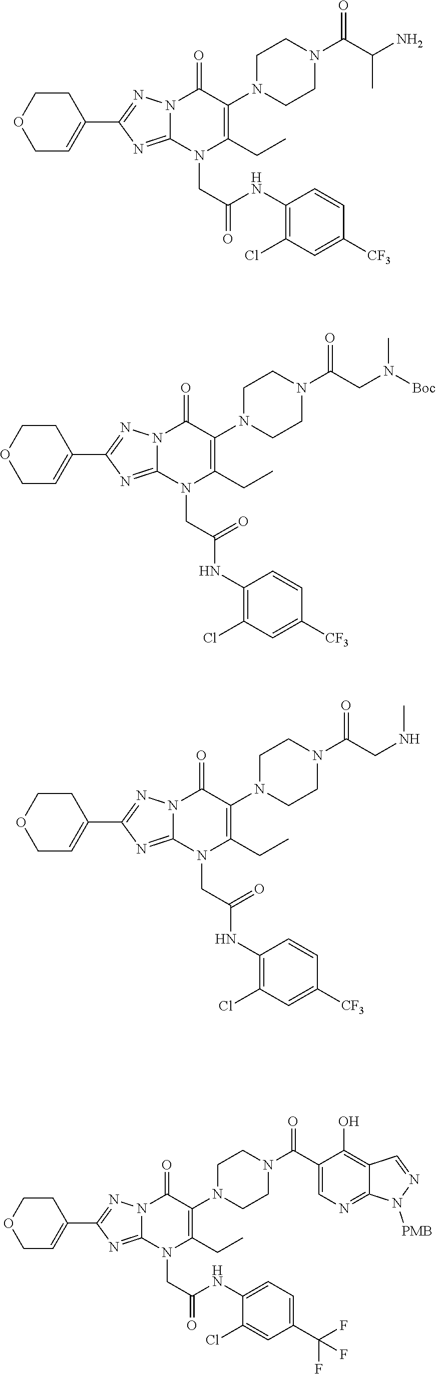

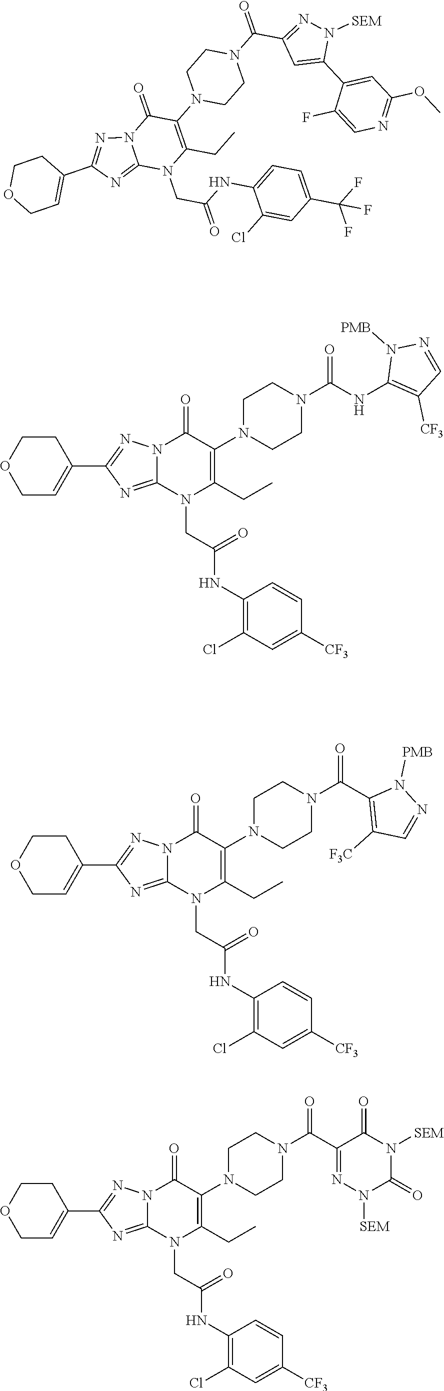

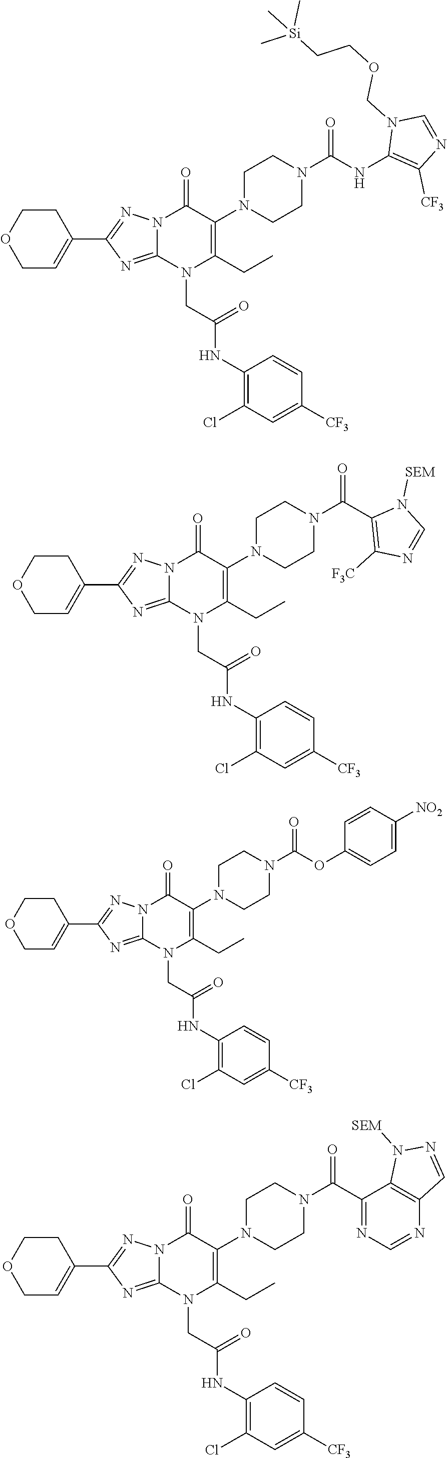 [SMILES: CCc1c(N2CCN(C(=O)Nc3c(C(F)(F)F)ncn3COCC[Si](C)(C)C)CC2)c(=O)n2nc(C3=CCOCC3)nc2n1CC(=O)Nc1ccc(C(F)(F)F)cc1Cl ; CCc1c(N2CCN(C(=O)c3c(C(F)(F)F)ncn3COCC[Si](C)(C)C)CC2)c(=O)n2nc(C3=CCOCC3)nc2n1CC(=O)Nc1ccc(C(F)(F)F)cc1Cl ; CCc1c(N2CCN(C(=O)Oc3ccc([N+](=O)[O-])cc3)CC2)c(=O)n2nc(C3=CCOCC3)nc2n1CC(=O)Nc1ccc(C(F)(F)F)cc1Cl ; CCc1c(N2CCN(C(=O)c3ncnc4cnn(COCC[Si](C)(C)C)c34)CC2)c(=O)n2nc(C3=CCOCC3)nc2n1CC(=O)Nc1ccc(C(F)(F)F)cc1Cl]

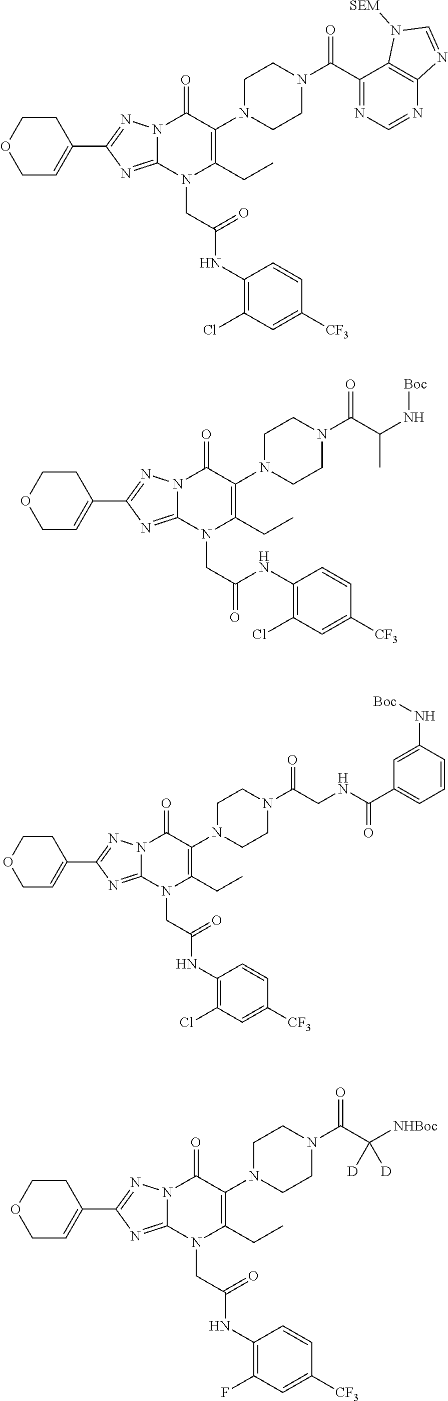 [SMILES: CCc1c(N2CCN(C(=O)c3ncnc4ncn(COCC[Si](C)(C)C)c34)CC2)c(=O)n2nc(C3=CCOCC3)nc2n1CC(=O)Nc1ccc(C(F)(F)F)cc1Cl ; CCc1c(N2CCN(C(=O)C(C)NC(=O)OC(C)(C)C)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC(=O)OC(C)(C)C)c3)CC2)c(=O)n2nc(C3=CCOCC3)nc2n1CC(=O)Nc1ccc(C(F)(F)F)cc1Cl ; [2H]C([2H])(NC(=O)OC(C)(C)C)C(=O)N1CCN(c2c(CC)n(CC(=O)Nc3ccc(C(F)(F)F)cc3F)c3nc(C4=CCOCC4)nn3c2=O)CC1]

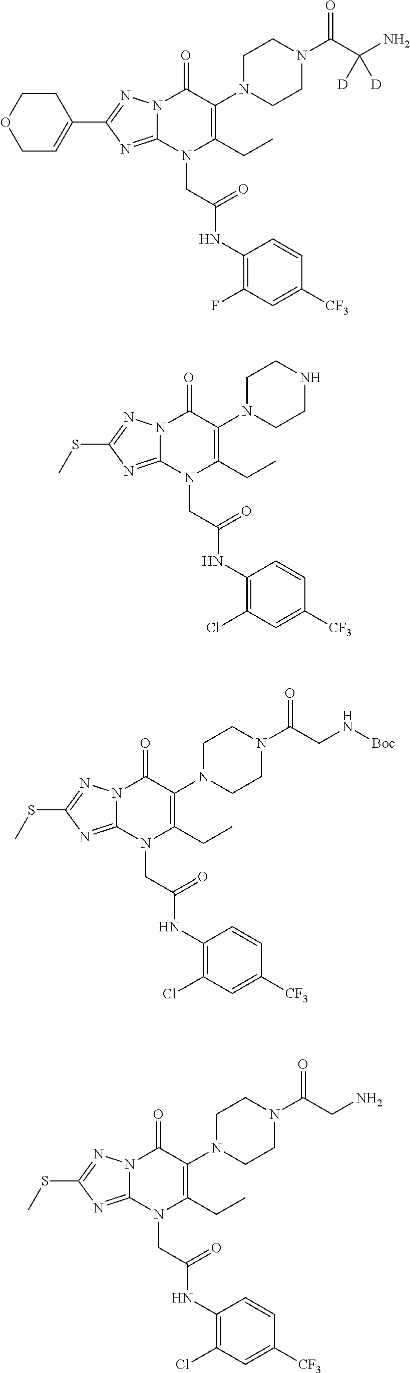 [SMILES: [2H]C([2H])(N)C(=O)N1CCN(c2c(CC)n(CC(=O)Nc3ccc(C(F)(F)F)cc3F)c3nc(C4=CCOCC4)nn3c2=O)CC1 ; CCc1c(N2CCNCC2)c(=O)n2nc(SC)nc2n1CC(=O)Nc1ccc(C(F)(F)F)cc1Cl ; CCc1c(N2CCN(C(=O)CNC(=O)OC(C)(C)C)CC2)c(=O)n2nc(SC)nc2n1CC(=O)Nc1ccc(C(F)(F)F)cc1Cl ; CCc1c(N2CCN(C(=O)CN)CC2)c(=O)n2nc(SC)nc2n1CC(=O)Nc1ccc(C(F)(F)F)cc1Cl]

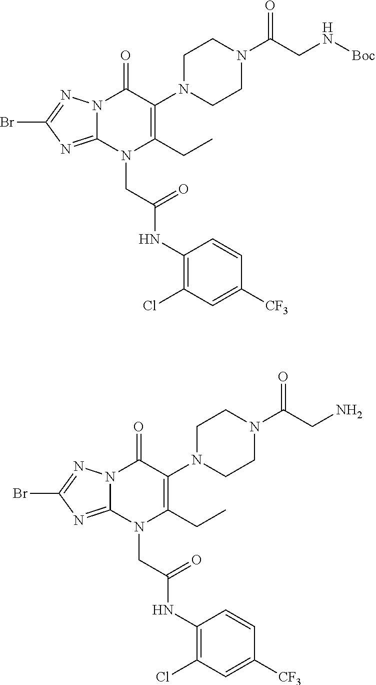 [SMILES: CCc1c(N2CCN(C(=O)CNC(=O)OC(C)(C)C)CC2)c(=O)n2nc(Br)nc2n1CC(=O)Nc1ccc(C(F)(F)F)cc1Cl ; CCc1c(N2CCN(C(=O)CN)CC2)c(=O)n2nc(Br)nc2n1CC(=O)Nc1ccc(C(F)(F)F)cc1Cl]

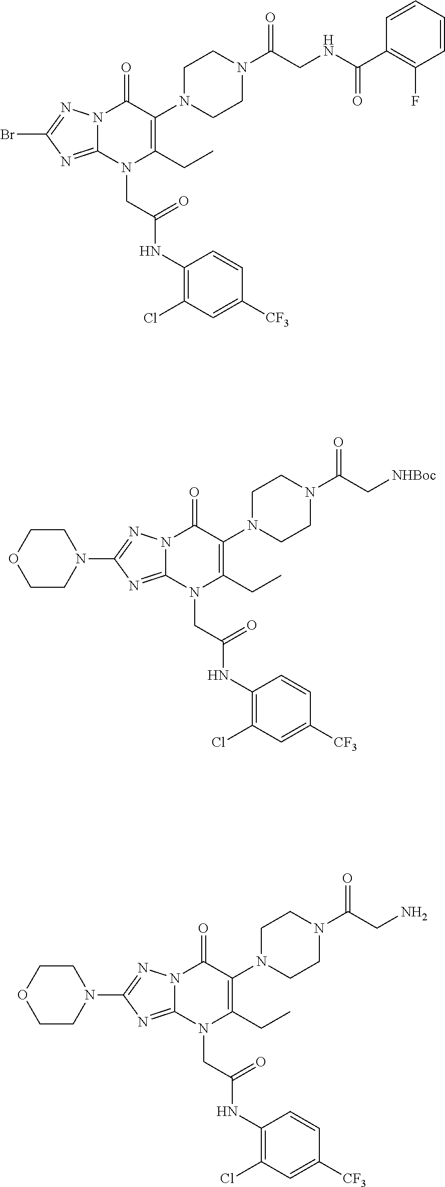 [SMILES: CCc1c(N2CCN(C(=O)CNC(=O)c3ccccc3F)CC2)c(=O)n2nc(Br)nc2n1CC(=O)Nc1ccc(C(F)(F)F)cc1Cl ; CCc1c(N2CCN(C(=O)CNC(=O)OC(C)(C)C)CC2)c(=O)n2nc(N3CCOCC3)nc2n1CC(=O)Nc1ccc(C(F)(F)F)cc1Cl ; CCc1c(N2CCN(C(=O)CN)CC2)c(=O)n2nc(N3CCOCC3)nc2n1CC(=O)Nc1ccc(C(F)(F)F)cc1Cl]

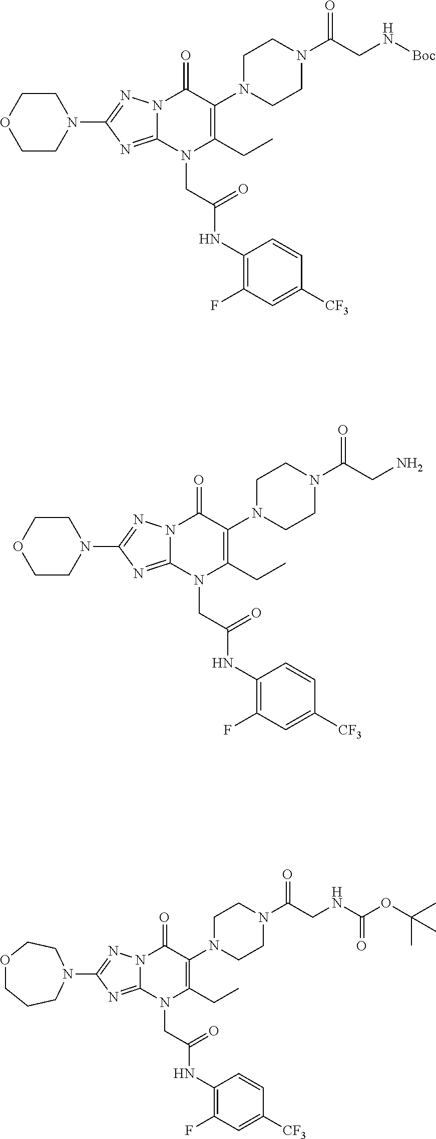 [SMILES: CCc1c(N2CCN(C(=O)CNC(=O)OC(C)(C)C)CC2)c(=O)n2nc(N3CCOCC3)nc2n1CC(=O)Nc1ccc(C(F)(F)F)cc1F ; CCc1c(N2CCN(C(=O)CN)CC2)c(=O)n2nc(N3CCOCC3)nc2n1CC(=O)Nc1ccc(C(F)(F)F)cc1F ; CCc1c(N2CCN(C(=O)CNC(=O)OC(C)(C)C)CC2)c(=O)n2nc(N3CCCOCC3)nc2n1CC(=O)Nc1ccc(C(F)(F)F)cc1F]

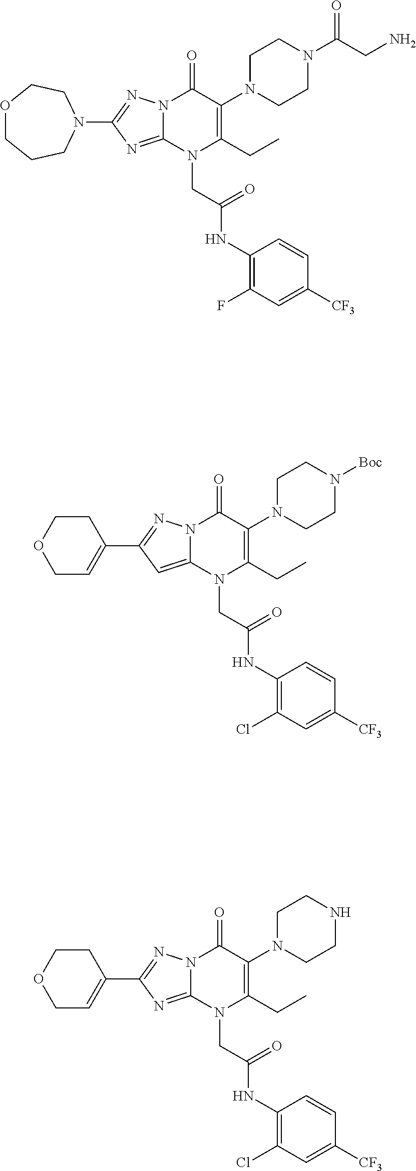 [SMILES: CCc1c(N2CCN(C(=O)CN)CC2)c(=O)n2nc(N3CCCOCC3)nc2n1CC(=O)Nc1ccc(C(F)(F)F)cc1F ; CCc1c(N2CCN(C(=O)OC(C)(C)C)CC2)c(=O)n2nc(C3=CCOCC3)cc2n1CC(=O)Nc1ccc(C(F)(F)F)cc1Cl ; CCc1c(N2CCNCC2)c(=O)n2nc(C3=CCOCC3)nc2n1CC(=O)Nc1ccc(C(F)(F)F)cc1Cl]

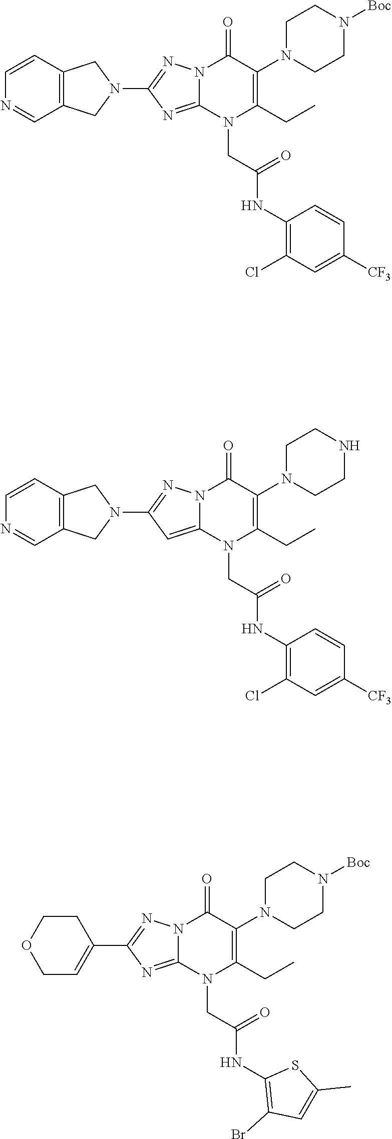 [SMILES: CCc1c(N2CCN(C(=O)OC(C)(C)C)CC2)c(=O)n2nc(N3Cc4ccncc4C3)nc2n1CC(=O)Nc1ccc(C(F)(F)F)cc1Cl ; CCc1c(N2CCNCC2)c(=O)n2nc(N3Cc4ccncc4C3)cc2n1CC(=O)Nc1ccc(C(F)(F)F)cc1Cl ; CCc1c(N2CCN(C(=O)OC(C)(C)C)CC2)c(=O)n2nc(C3=CCOCC3)nc2n1CC(=O)Nc1sc(C)cc1Br]

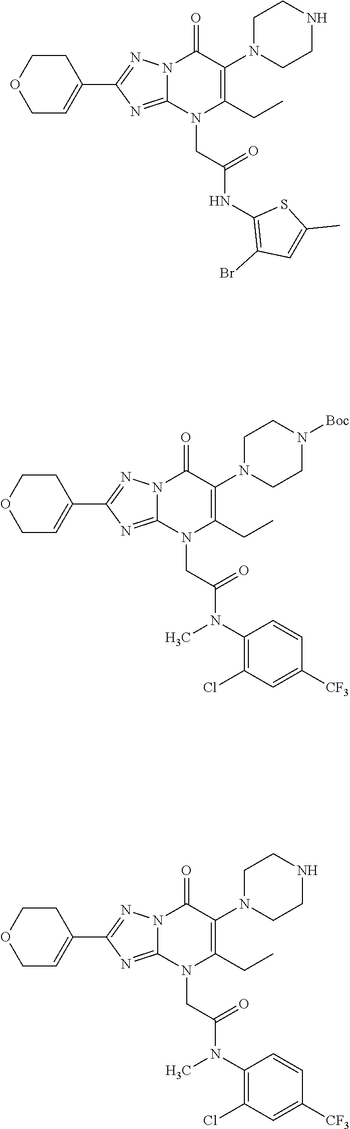 [SMILES: CCc1c(N2CCNCC2)c(=O)n2nc(C3=CCOCC3)nc2n1CC(=O)Nc1sc(C)cc1Br ; CCc1c(N2CCN(C(=O)OC(C)(C)C)CC2)c(=O)n2nc(C3=CCOCC3)nc2n1CC(=O)N(C)c1ccc(C(F)(F)F)cc1Cl ; CCc1c(N2CCNCC2)c(=O)n2nc(C3=CCOCC3)nc2n1CC(=O)N(C)c1ccc(C(F)(F)F)cc1Cl]

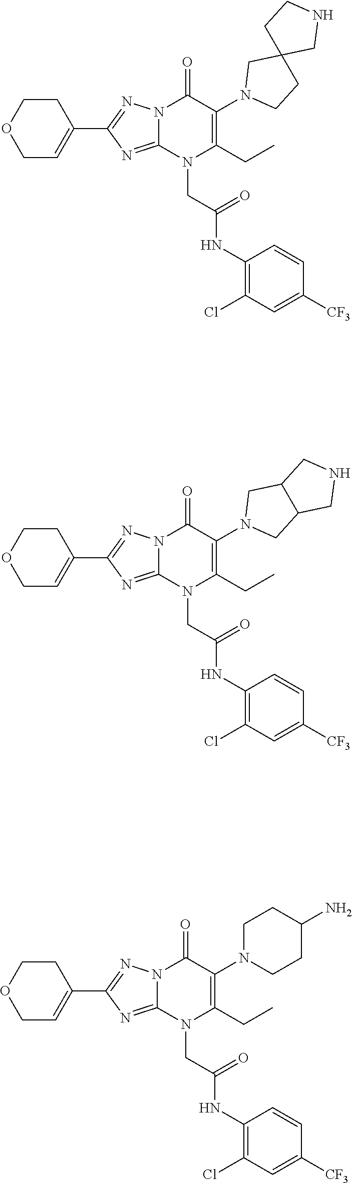 [SMILES: CCc1c(N2CCC3(CCNC3)C2)c(=O)n2nc(C3=CCOCC3)nc2n1CC(=O)Nc1ccc(C(F)(F)F)cc1Cl ; CCc1c(N2CC3CNCC3C2)c(=O)n2nc(C3=CCOCC3)nc2n1CC(=O)Nc1ccc(C(F)(F)F)cc1Cl ; CCc1c(N2CCC(N)CC2)c(=O)n2nc(C3=CCOCC3)nc2n1CC(=O)Nc1ccc(C(F)(F)F)cc1Cl]

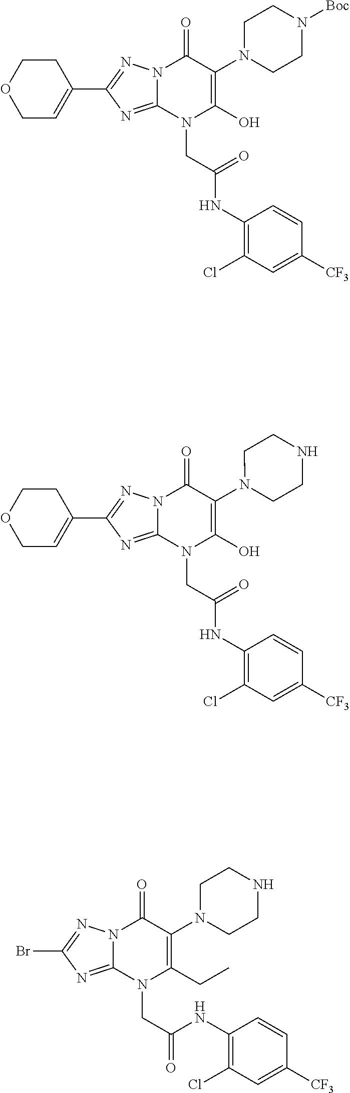 [SMILES: CC(C)(C)OC(=O)N1CCN(c2c(O)n(CC(=O)Nc3ccc(C(F)(F)F)cc3Cl)c3nc(C4=CCOCC4)nn3c2=O)CC1 ; O=C(Cn1c(O)c(N2CCNCC2)c(=O)n2nc(C3=CCOCC3)nc12)Nc1ccc(C(F)(F)F)cc1Cl ; CCc1c(N2CCNCC2)c(=O)n2nc(Br)nc2n1CC(=O)Nc1ccc(C(F)(F)F)cc1Cl]

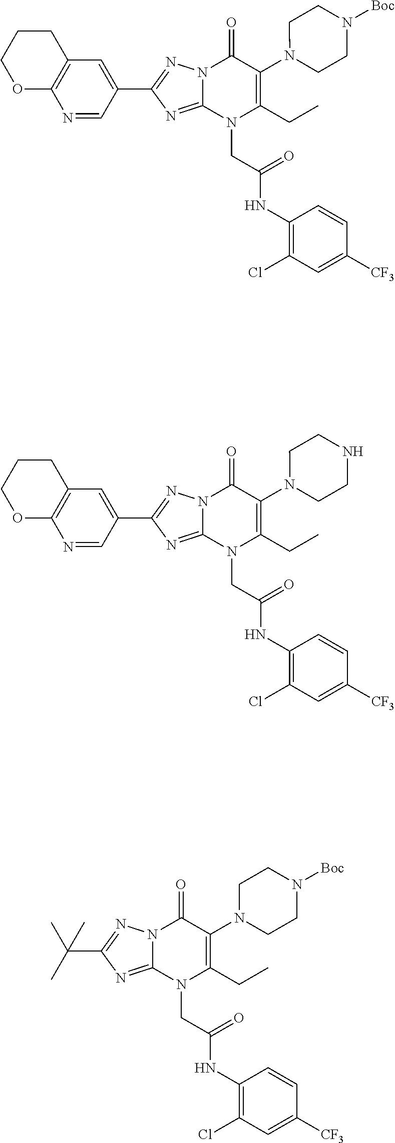 [SMILES: CCc1c(N2CCN(C(=O)OC(C)(C)C)CC2)c(=O)n2nc(-c3cnc4c(c3)CCCO4)nc2n1CC(=O)Nc1ccc(C(F)(F)F)cc1Cl ; CCc1c(N2CCNCC2)c(=O)n2nc(-c3cnc4c(c3)CCCO4)nc2n1CC(=O)Nc1ccc(C(F)(F)F)cc1Cl ; CCc1c(N2CCN(C(=O)OC(C)(C)C)CC2)c(=O)n2nc(C(C)(C)C)nc2n1CC(=O)Nc1ccc(C(F)(F)F)cc1Cl]

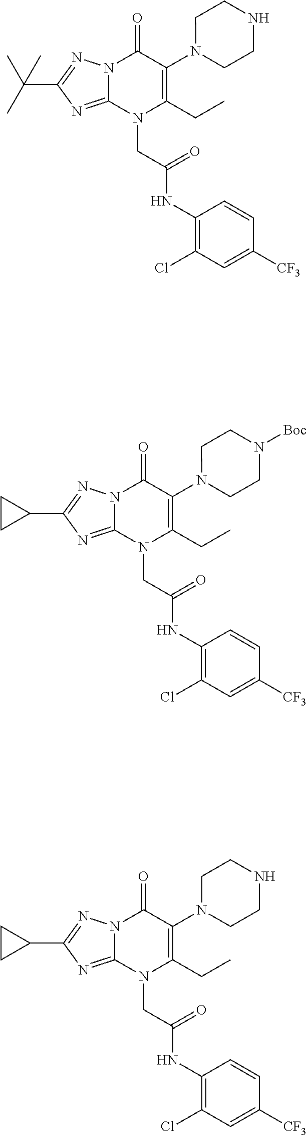 [SMILES: CCc1c(N2CCNCC2)c(=O)n2nc(C(C)(C)C)nc2n1CC(=O)Nc1ccc(C(F)(F)F)cc1Cl ; CCc1c(N2CCN(C(=O)OC(C)(C)C)CC2)c(=O)n2nc(C3CC3)nc2n1CC(=O)Nc1ccc(C(F)(F)F)cc1Cl ; CCc1c(N2CCNCC2)c(=O)n2nc(C3CC3)nc2n1CC(=O)Nc1ccc(C(F)(F)F)cc1Cl]

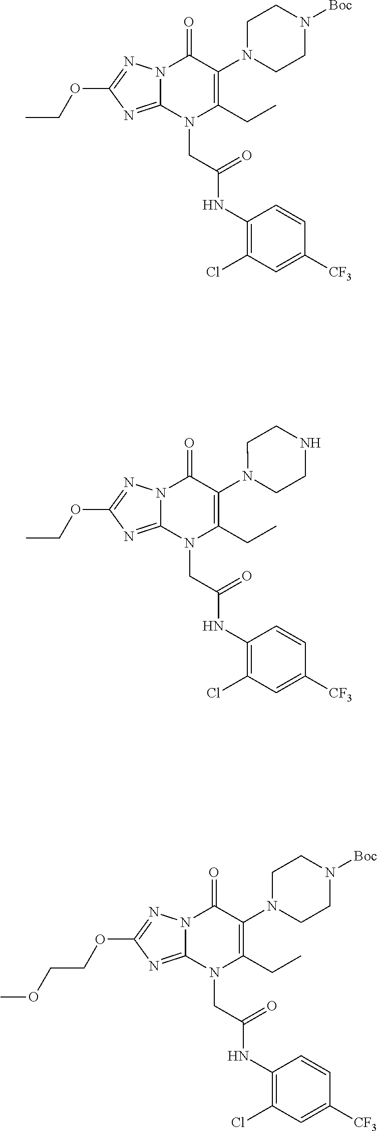 [SMILES: CCOc1nc2n(CC(=O)Nc3ccc(C(F)(F)F)cc3Cl)c(CC)c(N3CCN(C(=O)OC(C)(C)C)CC3)c(=O)n2n1 ; CCOc1nc2n(CC(=O)Nc3ccc(C(F)(F)F)cc3Cl)c(CC)c(N3CCNCC3)c(=O)n2n1 ; CCc1c(N2CCN(C(=O)OC(C)(C)C)CC2)c(=O)n2nc(OCCOC)nc2n1CC(=O)Nc1ccc(C(F)(F)F)cc1Cl]

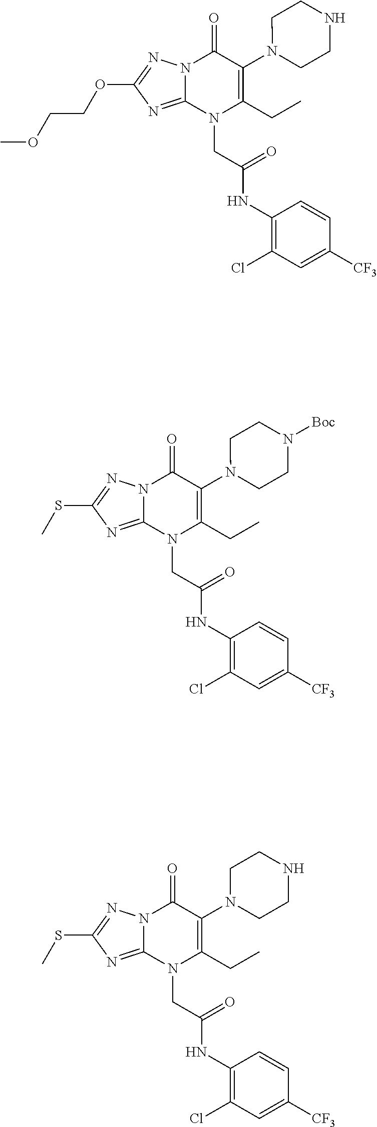 [SMILES: CCc1c(N2CCNCC2)c(=O)n2nc(OCCOC)nc2n1CC(=O)Nc1ccc(C(F)(F)F)cc1Cl ; CCc1c(N2CCN(C(=O)OC(C)(C)C)CC2)c(=O)n2nc(SC)nc2n1CC(=O)Nc1ccc(C(F)(F)F)cc1Cl ; CCc1c(N2CCNCC2)c(=O)n2nc(SC)nc2n1CC(=O)Nc1ccc(C(F)(F)F)cc1Cl]

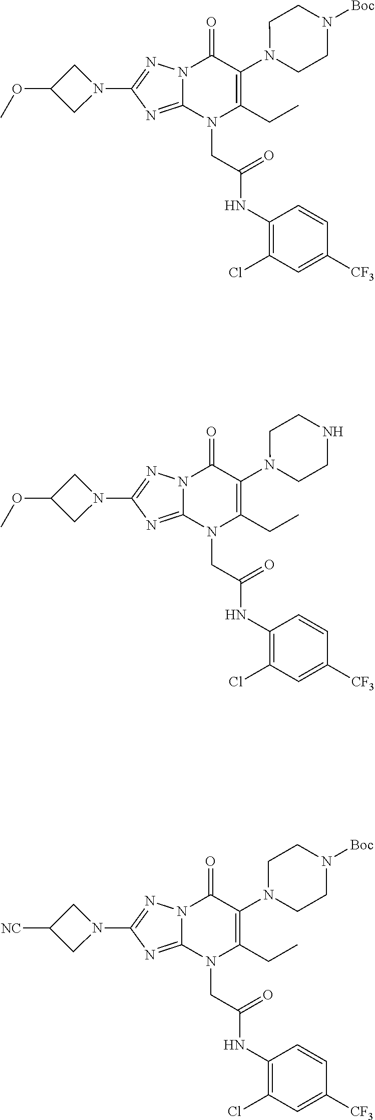 [SMILES: CCc1c(N2CCN(C(=O)OC(C)(C)C)CC2)c(=O)n2nc(N3CC(OC)C3)nc2n1CC(=O)Nc1ccc(C(F)(F)F)cc1Cl ; CCc1c(N2CCNCC2)c(=O)n2nc(N3CC(OC)C3)nc2n1CC(=O)Nc1ccc(C(F)(F)F)cc1Cl ; CCc1c(N2CCN(C(=O)OC(C)(C)C)CC2)c(=O)n2nc(N3CC(C#N)C3)nc2n1CC(=O)Nc1ccc(C(F)(F)F)cc1Cl]

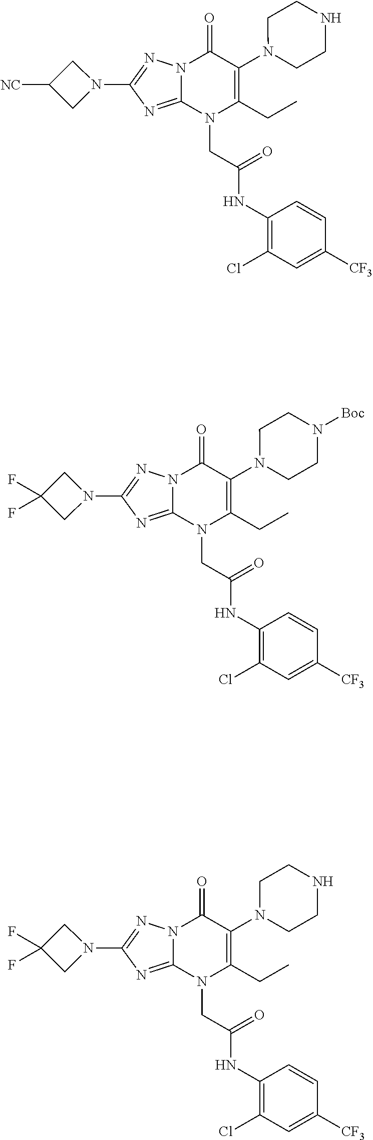 [SMILES: CCc1c(N2CCNCC2)c(=O)n2nc(N3CC(C#N)C3)nc2n1CC(=O)Nc1ccc(C(F)(F)F)cc1Cl ; CCc1c(N2CCN(C(=O)OC(C)(C)C)CC2)c(=O)n2nc(N3CC(F)(F)C3)nc2n1CC(=O)Nc1ccc(C(F)(F)F)cc1Cl ; CCc1c(N2CCNCC2)c(=O)n2nc(N3CC(F)(F)C3)nc2n1CC(=O)Nc1ccc(C(F)(F)F)cc1Cl]

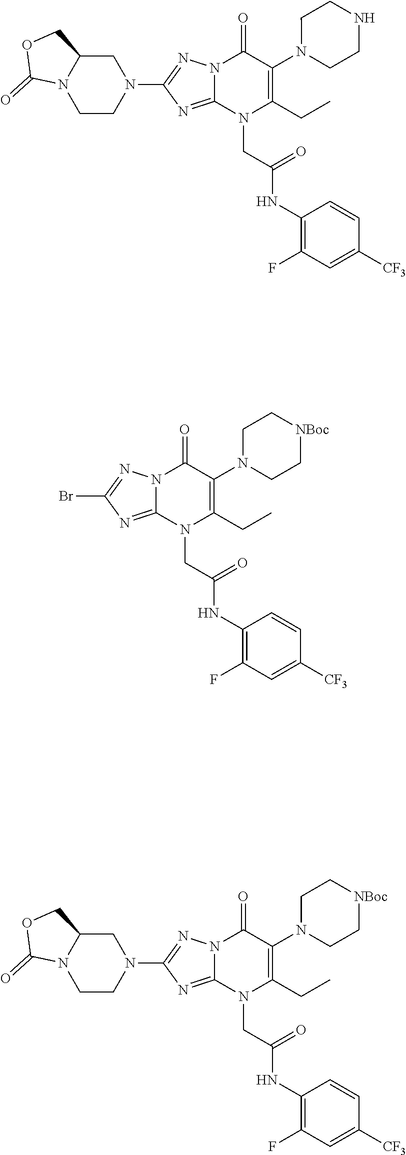 [SMILES: CCc1c(N2CCNCC2)c(=O)n2nc(N3CCN4C(=O)OC[C@H]4C3)nc2n1CC(=O)Nc1ccc(C(F)(F)F)cc1F ; CCc1c(N2CCN(C(=O)OC(C)(C)C)CC2)c(=O)n2nc(Br)nc2n1CC(=O)Nc1ccc(C(F)(F)F)cc1F ; CCc1c(N2CCN(C(=O)OC(C)(C)C)CC2)c(=O)n2nc(N3CCN4C(=O)OC[C@H]4C3)nc2n1CC(=O)Nc1ccc(C(F)(F)F)cc1F]

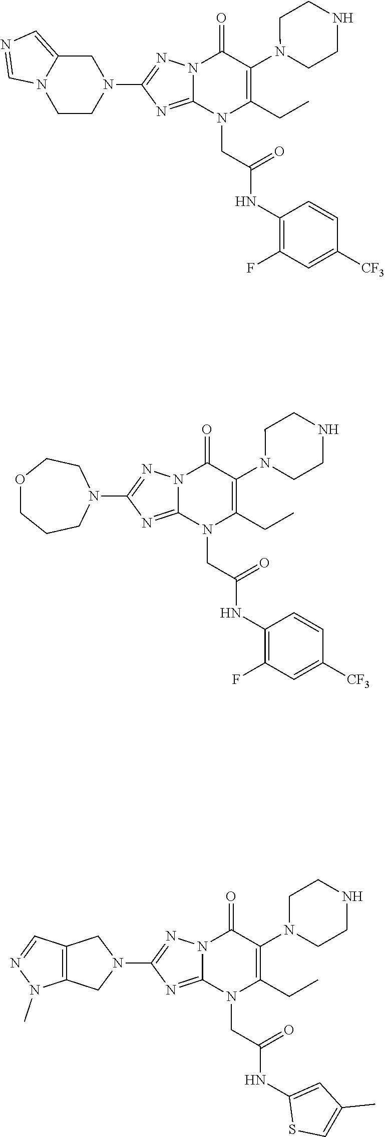 [SMILES: CCc1c(N2CCNCC2)c(=O)n2nc(N3CCn4cncc4C3)nc2n1CC(=O)Nc1ccc(C(F)(F)F)cc1F ; CCc1c(N2CCNCC2)c(=O)n2nc(N3CCCOCC3)nc2n1CC(=O)Nc1ccc(C(F)(F)F)cc1F ; CCc1c(N2CCNCC2)c(=O)n2nc(N3Cc4cnn(C)c4C3)nc2n1CC(=O)Nc1cc(C)cs1]

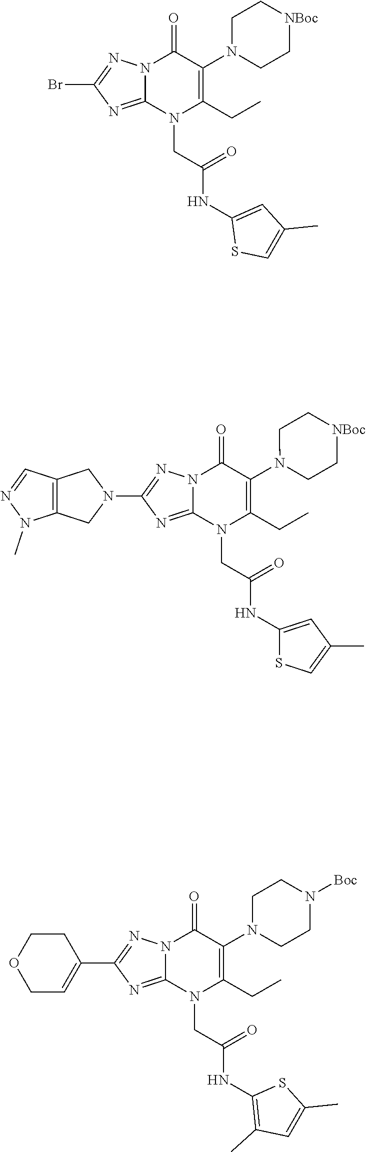 [SMILES: CCc1c(N2CCN(C(=O)OC(C)(C)C)CC2)c(=O)n2nc(Br)nc2n1CC(=O)Nc1cc(C)cs1 ; CCc1c(N2CCN(C(=O)OC(C)(C)C)CC2)c(=O)n2nc(N3Cc4cnn(C)c4C3)nc2n1CC(=O)Nc1cc(C)cs1 ; CCc1c(N2CCN(C(=O)OC(C)(C)C)CC2)c(=O)n2nc(C3=CCOCC3)nc2n1CC(=O)Nc1sc(C)cc1C]

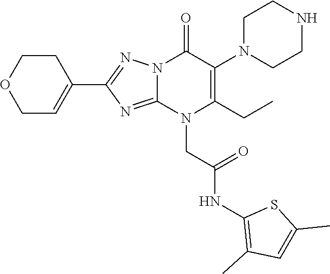 [SMILES: CCc1c(N2CCNCC2)c(=O)n2nc(C3=CCOCC3)nc2n1CC(=O)Nc1sc(C)cc1C]

or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

The present disclosure further provides compounds described herein, or a pharmaceutically acceptable salt thereof, for use in any of the methods described herein. The present disclosure further provides uses of a compound described herein, or a pharmaceutically acceptable salt thereof, for the preparation of a medicament for use in any of the methods described herein.

The present disclosure further provides pharmaceutical compositions comprising a compound described herein, or a pharmaceutically acceptable salt thereof, and a pharmaceutically acceptable carrier.

The invention provides a method of inhibiting WRN in a cell expressing WRN, the method comprising contacting the cell with the compound disclosed herein.

In some embodiments, the cell is associated with microsatellite instability-high (MSI-H) or mismatch repair deficient (dMMR). In some embodiments, the cell is in a subject.

The invention provides a method of treating a subject in need thereof comprising administering to the subject the compound disclosed herein, or a pharmaceutically acceptable salt thereof, or the pharmaceutical composition disclosed herein.

In some embodiments, the subject is suffering from, and is in need of a treatment for, a disease or condition having the symptom of dMMR/MSI-H. In some embodiments, the disease or condition is a cancer. In some embodiments, the cancer is a cancer with microsatellite instability-high (MSI-H) or mismatch repair deficient (dMMR).

A method of inhibiting WRN in a subject, wherein the method comprises administering to the subject a therapeutically effective amount of the present invention, or a pharmaceutically acceptable salt thereof, or the pharmaceutical composition of the present invention.

A method of treating a disease which can be treated by WRN inhibition in a subject, comprising administering to the subject a therapeutically effective amount of the compound of the present invention, or a pharmaceutically acceptable salt thereof, or the pharmaceutical composition of the present invention.

In some embodiments, the disease is cancer.

In some embodiments, the cancer is characterized as MSI-H or dMMR.

In some embodiments, the cancer characterized as MSI-H or dMMR is colorectal, gastric, prostate, endometrial, adrenocortical, uterine, cervical, esophageal, breast, kidney and ovarian cancer.

In some embodiments, the cancer characterized as MSI-H or dMMR is colorectal, gastric and endometrial cancer.

In some embodiments, the cancer characterized as microsatellite instability-high (MSI-H) or mismatch repair deficient (dMMR) is prostate cancer, uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

Use of the compound of the present invention or a pharmaceutically acceptable salt thereof or the pharmaceutical composition of the present invention, in the manufacture of a medicament for the treatment of cancer.

In some embodiments, the cancer is characterized as microsatellite instability-high (MSI-H) or mismatch repair deficient (dMMR).

Routs of administration for the compounds in the present disclosure include, but not limited to oral, injection, topical and inhalation.

### Definitions

Unless other indicated, the following terms are intended to have the meaning set forth below. Other terms are defined elsewhere throughout the specification.

As used herein, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology such as “solely”, “only” and the like in connection with the recitation of claim elements, or use of a “negative” limitation.

At various places in the present specification, variables defining divalent linking groups are described. It is specifically intended that each linking substituent include both the forward and backward forms of the linking substituent. For example, —NR(CR′R″)— includes both —NR(CR′R″)— and —(CR′R″)NR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cn-Cm” indicates a range which includes the endpoints, wherein n and m are integers and indicate the number of carbons. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

As used herein, unless otherwise indicated, the term “alkyl”, by itself or as part of another substituent, is meant to refer to a saturated hydrocarbon group which is straight-chained or branched. An alkyl group can contain from 1 to about 20, from 2 to about 20, from 1 to about 10, from 1 to about 8, from 1 to about 6, from 1 to about 4, or from 1 to about 3 carbon atoms. Similarly, C₁-8, as in C₁-8 alkyl is defined to identify the group as having 1, 2, 3, 4, 5, 6, 7 or 8 carbon atoms in a linear or branched arrangement. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

As used herein, unless otherwise indicated, “alkenyl” refers to an alkyl group having one or more double carbon-carbon bonds. Example alkenyl groups include, but are not limited to, ethenyl, propenyl, and the like.

As used herein, unless otherwise indicated, “alkynyl” refers to an alkyl group having one or more triple carbon-carbon bonds. Example alkynyl groups include, but are not limited to, ethynyl, propynyl, and the like.

As used herein, unless otherwise indicated, “haloalkyl” refers to an alkyl group having one or more halogen substituents. Example haloalkyl groups include, but are not limited to, CF₃, C₂F₅, CHF₂, CH₂F, CCl₃, CHCl₂, C₂Cl₅, and the like.

As used herein, unless otherwise indicated, “aryl” refers to an unsubstituted or substituted monocyclic or polycyclic (e.g., having 2, 3 or 4 fused rings) aromatic hydrocarbons. In some embodiments, aryl groups have from 6 to about 20 carbon atoms. In some embodiments, aryl groups have from 6 to about 14 carbon atoms. In some embodiments, aryl groups have from 6 to about 10 carbon atoms. Example aryl groups include, but are not limited to, phenyl, naphthyl, anthracenyl, phenanthrenyl, indanyl, indenyl, and the like.

As used herein, unless otherwise indicated, “cycloalkyl” refers to an unsubstituted or substituted non-aromatic carbocycles including cyclized alkyl, alkenyl, and alkynyl groups. Cycloalkyl groups can include mono- or polycyclic (e.g., having 2, 3 or 4 fused rings) ring systems, including fused rings, spirocyclic rings, and bridged rings (e.g., a bridged bicycloalkyl group). In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Cycloalkyl groups can be optionally substituted by oxo or sulfido (e.g., —C(O)— or —C(S)—). Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. In some embodiments, the cycloalkyl is a C₃-C₇ monocyclic cycloalkyl. In some embodiments, the cycloalkyl is a C₄-C₁₀ spirocycle or bridged cycloalkyl. Example cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. In some embodiments, cycloalkyl are cyclic-containing, non-aromatic hydrocarbon groups having from 3 to 12 carbon atoms (“C₃-C₁₂”), preferably from 3 to 6 carbon atoms (“C₃-C₆”). Examples of cycloalkyl groups include, for example, cyclopropyl (C₃:3-membered), cyclobutyl (C₄:4-membered), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “spirocycloalkyl” when used alone or as part of a substituent group refers to a non-aromatic hydrocarbon group containing two cycloalkyl rings, and wherein the two cycloalkyl rings share a single carbon atom in common.

As used herein, unless otherwise indicated, a “heteroaryl” group refers to an unsubstituted or substituted aromatic heterocycle having at least one heteroatom ring member such as boron, sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, unless otherwise indicated, “heterocycloalkyl” refers to an unsubstituted or substituted monocyclic (saturated or partially unsaturated ring) or polycyclic heterocycles having at least one non-aromatic ring (saturated or partially unsaturated ring), wherein one or more of the ring-forming carbon atoms of the heterocycloalkyl is replaced by a heteroatom selected from N, O, S and B, and wherein the ring-forming carbon atoms and heteroatoms of the heterocycloalkyl group can be optionally substituted by one or more oxo or sulfido (e.g., C(O), S(O), C(S), or S(O)₂, etc.).

Heterocycloalkyl groups include monocyclic and polycyclic (e.g., having 2 fused rings) systems. Included in heterocycloalkyl are monocyclic and polycyclic 3-10, 4-10, 3-7, 4-7, and 5-6 membered heterocycloalkyl groups. Heterocycloalkyl groups can also include spirocycles and bridged rings (e.g., a 5-10 membered bridged biheterocycloalkyl ring having one or more of the ring-forming carbon atoms replaced by a heteroatom independently selected from N, O, S and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members.

Example heterocycloalkyl groups include, but are not limited to, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, diazabicyclo[2.2.2]octanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, oxa-azaspiro[3.5]nonanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, octahydropyrrolo[3,4-c]pyrrolyl and the like.

In some embodiments, heterocycloalkyl refers to any three to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

In some embodiments, the term “spiroheterocycloalkyl” when used alone or as part of a substituent group refers to a non-aromatic group containing two rings, at least one of which is a heterocycloalkyl ring, and wherein the two rings share a single carbon atom in common.

As used herein, unless otherwise indicated, “arylcycloalkyl” refers to cycloalkyl group substituted by an aryl group.

As used herein, unless otherwise indicated, “arylheterocycloalkyl” refers to a heterocycloalkyl group substituted by an aryl group.

As used herein, unless otherwise indicated, “arylheteroaryl” refers to a heteroaryl group substituted by an aryl group.

As used herein, unless otherwise indicated, “biaryl” refers to an aryl group substituted by another aryl group.

As used herein, unless otherwise indicated, “heteroarylcycloalkyl” refers to a cycloalkyl group substituted by a heteroaryl group.

As used herein, unless otherwise indicated, “heteroarylheterocycloalkyl” refers to a heterocycloalkyl group substituted by a heteroaryl group.

As used herein, unless otherwise indicated, “heteroarylaryl” refers to an aryl group substituted by a heteroaryl group.

As used herein, unless otherwise indicated, “biheteroaryl” refers to a heteroaryl group substituted by another heteroaryl group.

As used herein, “halo” or “halogen” includes fluoro, chloro, bromo, and iodo.

As used herein, unless otherwise indicated, “alkoxy” refers to an —O-alkyl group. Example alkoxy groups include methoxy, ethoxy, propoxy (e.g., n-propoxy and isopropoxy), t-butoxy, and the like.

As used herein, unless otherwise indicated, “hydroxylalkyl” refers to an alkyl group substituted by OH.

As used herein, unless otherwise indicated, “cyanoalkyl” refers to an alkyl group substituted by CN.

As used herein, unless otherwise indicated, “alkoxyalkyl” refers to an alkyl group substituted by an alkoxy group.

As used herein, unless otherwise indicated, “alkoxyalkoxy” refers to an alkoxy group substituted by alkoxy.

As used herein, unless otherwise indicated, “haloalkoxy” refers to an —O-(haloalkyl) group.

As used herein, unless otherwise indicated, “arylalkyl” refers to alkyl substituted by aryl and “cycloalkylalkyl” refers to alkyl substituted by cycloalkyl. An example arylalkyl group is benzyl.

As used herein, unless otherwise indicated, “heteroarylalkyl” refers to alkyl substituted by heteroaryl and “heterocycloalkylalkyl” refers to alkyl substituted by heterocycloalkyl.

As used herein, unless otherwise indicated, “oxo” refers to an oxygen substituent that is connected by a double bond (i.e., ═O).

As used herein, unless otherwise indicated, the phrase “optionally substituted” means unsubstituted or substituted.

As used herein, unless otherwise indicated, the term “substituted” refers to a group in which one or more hydrogen atoms are each independently replaced with the same or different substituent(s). Typical substituents include, but are not limited to, H, D, halogen, CN, NO₂, SF₅, C₁-C₆ alkyl, C₂-C₆ alkenyl, C₂-C₆ alkynyl, C₃-C₆ cycloalkyl, 4-6 membered heterocycloalkyl, NR^{C}R^{D}, OR^{A}, SR^{A}, NHOR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, or OP(O)OR^{E}OR^{F}; wherein, C₁-C₆ alkyl, C₂-C₆ alkenyl, C₂-C₆ alkynyl, C₃-C₆ cycloalkyl, 4-6 membered heterocycloalkyl is optionally substituted by 1-6 substituents independently selected from D, halogen, CN, NO₂, SF₅, OH, oxo, C₁-C₆ alkyl, —O—C₁-C₆ alkyl, C₁-C₆ alkyl-OH, C₁-C₆ alkyl-CN, —OC₁-C₆ haloalkyl, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}.

The compounds described herein can be asymmetric (e.g., having one or more stereocenters). All stereoisomers, such as enantiomers and diastereomers, are intended unless otherwise indicated.

Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some cases, the compounds of the present disclosure may exist as rotational isomers. Descriptions of a compound of the invention that do not indicate a particular rotational isomer are intended to encompass any individual rotational isomers, as well as mixtures of rotational isomers in any proportion. Depiction of a particular rotational isomer is meant to refer to the depicted rotational isomer, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., entaniomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (2H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

As used herein, the term “inhibit”, “inhibition” or “inhibiting” refers to the reduction or suppression of a given condition, symptom, or disorder, or disease, or a significant decrease in the baseline activity of a biological activity or process.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. ‘WRN inhibitor’ or ‘WRN helicase inhibitor’ as used herein means a compound that inhibits Werner Syndrome RecQ DNA helicase (WRN).

The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

‘Disease or condition mediated by WRN’ includes a disease or condition, such as cancer, which is treated by WRN inhibition. In particular this can include cancers characterized as microsatellite instability-high (MSI-H) or mismatch repair deficient (dMMR).

‘Microsatellite unstable cancer’, microsatellite instability-high cancer’, ‘microsatellite high cancer’ and ‘MSI-high cancer’ ‘MSIhi’ and ‘MSI-H’ when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR.

The terms “synthetic lethality,” and “synthetic lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

### Pharmaceutical Compositions

Also provided are pharmaceutical compositions comprising compounds of Formula I, or a pharmaceutically acceptable salt, stereoisomer, solvate, tautomer, isotopic variant, prodrug, N-oxide or deuterated compound thereof, and a pharmaceutically acceptable carrier.

The compositions may be in a form suitable for oral use (for example as tablets, lozenges, hard or soft capsules, aqueous or oily suspensions, emulsions, dispersible powders or granules, syrups or elixirs), for injection use (for example as aqueous or oil suspensions, or emulsions, with sesame oil, corn oil, cottonseed oil, or peanut oil, as well as elixirs, mannitol, dextrose, or a sterile aqueous solution, and similar pharmaceutical vehicles), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions may be obtained by conventional procedures using conventional pharmaceutical excipients, well known in the art. Thus, compositions intended for oral use may contain, for example, one or more coloring, sweetening, flavoring and/or preservative agents.

An effective amount of a compound of Formula (I) or a pharmaceutically salt thereof for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.1 mg to 1000 mg of Formula (I) or a pharmaceutically salt thereof with an appropriate and convenient amount of excipients which may vary from about 5 to about 98 percent by weight of the total composition.

The size of the dose for therapeutic or prophylactic purposes of a compound of the Formula (I) will naturally vary according to the nature and severity of the conditions, the age and sex of the animal or patient and the route of administration, according to well-known principles of medicine.

Described below are non-limiting exemplary pharmaceutical compositions and methods for preparing the same.

### Methods of Administration

The compounds of Formula (I) or a pharmaceutically salt thereof or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC₅₀).

In some embodiments, the subject methods utilize a WRN inhibitor with an IC₅₀ value of about or less than a predetermined value, as ascertained in an in vitro assay. In some embodiments, the WRN inhibitor inhibits WRN with an IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 UM or less, 1.7 UM or less, 1.8 UM or less, 1.9 μM or less, 2 μM or less, 5 μM or less, 10 μM or less, 15 μM or less, 20 μM or less, 25 UM or less, 30 μM or less, 40 UM or less, 50 μM, 60 μM, 70 μM, 80μ, 90 μM, 100 μM, 200 μM, 300 μM, 400 μM, or 500 μM, or less, (or a number in the range defined by and including any two numbers above).

WRN is a synthetic lethal target in the cancers with microsatellite instability-high (MSI-H) or mismatch repair deficient (dMMR). The subject methods are useful for treating disease conditions associated with microsatellite instability-high (MSI-H) or mismatch repair deficient (dMMR).

A cancer that has “defective mismatch repair” (dMMR) or “dMMR character” includes cancer types associated with documented MLH1, PMS2, MSH2, MSH3, MSH4, MSH5, MSH6, MLH3, PMS1, and EXO1 mutations or epigenetic silencing, microsatellite fragile sites, or other gene inactivation mechanisms. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

In other embodiments, said method is for treating a disease or cancer selected from the cancers with poor chemotherapy response or chemotherapy resistance.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other methods, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith el ah, March's Advanced Organic Chemistry: Reactions, Mechanisms, and Structure, 8th Ed. (Wiley, 2019); Peturssion et al, “Protecting Groups in Carbohydrate Chemistry,” J Chem. Educ., 1997, 74(11), 1297; and Wuts et al., Protective Groups in Organic Synthesis, 5th Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature”, “room temperature”, and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### General Synthetic Procedures

A series of intermediates of formula I-SM-A for preparations of formula I can be prepared by the method outlined in Scheme 1. Condensations of b-keto esters 1-1 where R^{W} is C₁-C₆ alkyl (such as Me or Et) with bromo-azole amine 1-2 where X¹ and X² is independently N or CR⁷ in the presence of an acid such as H₃PO₄ can afford bicyclic compounds 1-3. Suzuki coupling of the bicyclic compounds 1-3 with R¹W¹ 1-4 where W¹ is boronic acid or boronic ester group can afford the corresponding bicyclic compounds 1-5 under standard Suzuki conditions (e.g., in the presence of a palladium catalyst, such as [1,1′-bis(diphenylphosphino)ferrocene]dichloropalladium (II) and a base, such as K₃PO₄). Alternatively, the R¹ substituted bicyclic compounds 1-5 where R¹ is a secondary or tertiary amino group can be obtained by Buchwald coupling with amine derivative R¹—W¹ (primary amine or secondary amine) under Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, Cs₂CO₃, or K₂CO₃. Alkylation of the NH in the bicyclic compounds 1-5 to provide the intermediates of formula I-SM-A can be achieved by reactions with a-chloroacetamides 1-6 under basic conditions using a base such as TEA or Hunig's base in a suitable solvent such as DMF. The a-chloroacetamides 1-6 can be conveniently obtained by reactions of suitable amine derivatives 1-8 with a-chloroacetic acids 1-7 under amide coupling conditions (e.g., in the presence of an amide coupling reagent such as BOP, PyBOP, HATU, HBTU or EDCI, and a base such as TEA, Hunig's base or pyridine) or with its chloride 1-9 in the presence of a base such as TEA or Hunig's base.

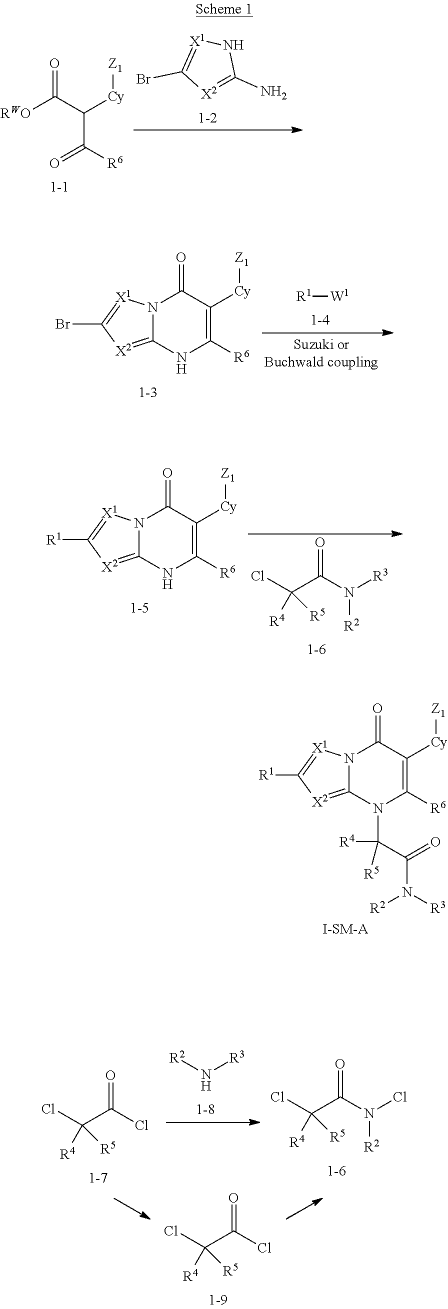

A series of intermediates of formula 2-5 for preparations of the intermediates I-SM-A can be prepared by the method outlined in Scheme 2. Condensations of the b-keto esters 2-1 where R^{W} is C₁-C₆ alkyl (such as Me or Et) with azole amine derivatives 2-2 where X¹ and X² is independently N or CR⁷ in the presence of an acid such as H₃PO₄ can afford the bicyclic intermediates 2-3. Negishi reactions of the bicyclic intermediates 2-3 with R¹-M (where M is Sn(Me)₃, Sn(Bu)₃, ZnCl, ZnBr or ZnI) can afford the bicyclic intermediates 2-5 under standard Negishi conditions (e.g., in the presence of a palladium catalyst, such as tetrakis(triphenylphosphine) palladium (0) or [1,1′-bis(diphenylphosphino)-ferrocene]dichloropalladium (II)), or standard Stille conditions (e.g., in the presence of a palladium(0) catalyst, such as tetrakis(triphenyl-phosphine) palladium (0)).

Alternatively, Suzuki coupling of bromo-azole amine derivatives 2-6 where X¹ and X² is independently N or CR⁷ with boronic acid or boronic esters R¹—W¹ 2-7 where W¹ is boronic acid or boronic ester groups can yield the corresponding azole amine derivatives 2-8 under standard Suzuki conditions (e.g., in the presence of a palladium catalyst, such as [1,1′-bis(diphenylphosphino) ferrocene]dichloropalladium (II) and a base, such as K₃PO₄). Condensations of the azole amine derivatives 2-8 with the b-keto esters 2-1 where R is alkyl (e.g., Me or Et) in the presence of an acid such as H₃PO₄ can afford the bicyclic intermediates 2-5.

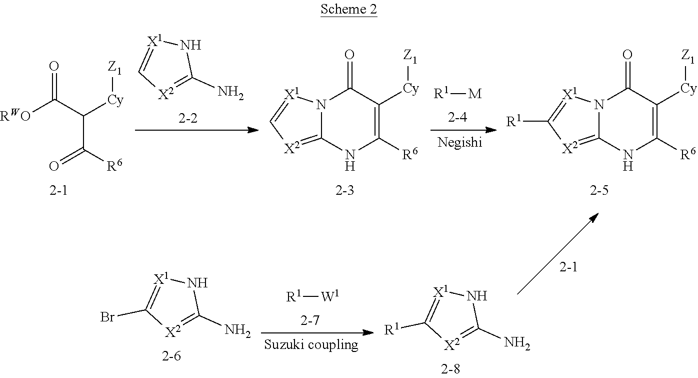

A series of b-keto ester intermediates of formula 3-4 for preparations of the intermediates I-SM-A can be prepared by the method outlined in Scheme 3. Claisen condensations of ester derivatives 3-1 where R^{W} is C₁-C₆ alkyl (such as Me or Et) with appropriate esters 3-3 where R^{6A} is C₁-C₆ alkyl (such as Me or Et) or acyl chlorides 3-2 to the b-keto esters 3-4 can be achieved under basic conditions such as NaOMe, NaOEt, LiHMDS, NaHMDS or LDA in a suitable solvent such as anhydrous THF or ether. Alternatively, the ester derivatives 3-1 can be transformed into silicane enols 3-5 which can react with acyl chlorides 3-2 to product the b-keto esters 3-4.

On the other way, the b-keto esters 3-4 can be prepared by arylations of aryl halides Z¹-Cy-W 1-7 (where Z¹ is a substituent, Cy is aryl or heteroaryl and W is halo (e.g., Cl, Br or I) or pseudohalogen (e.g., OTf or OMs)) with activated methylene b-keto esters 3-6 under copper-catalyzed Ullmann-type coupling conditions (e.g., in the presence of catalysis of CuI/L-proline or sarcosine in DMSO with a base such as Cs₂CO₃) or under Pd-catalyzed arylation conditions (e.g., in the presence of a Pd-catalysis such as Pd(OAc)₂ and 2-di-tert-butylphosphino-2′-methylbiphenyl with a base such as K₃PO₄ in toluene.

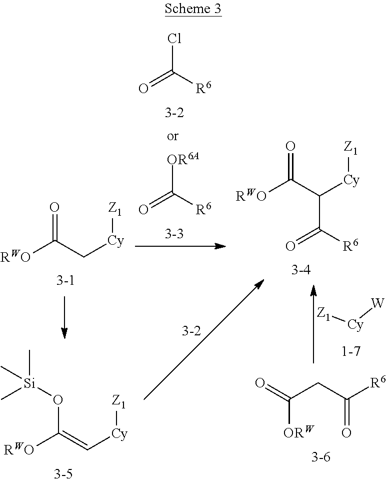

A series of intermediates of formula 4-5 for preparations of the intermediates I-SM-B can be prepared by the method outlined in Scheme 4. Cyclization of the b-keto esters 4-1 where R^{W} is C₁-C₆ alkyl (such as Me or Et) with hydrazinecarboximidamide hydrochloride 4-2 in the presence of a base such as sodium ethoxide in a suitable solvent such as ethanol can provide the cyclized products 4-3. Reactions of 4-3 with acyl chlorides 4-4, carboxylate esters 4-6 or carbodithioate esters 4-7 in a suitable solvent such as 1,4-dioxane, DMAC or DMF at enhanced temperature can yield the bicyclic compounds 4-5. Alternatively, reaction of 4-3 with suitable aldehydes R¹CHO 4-8 in the presence of FeCl₃ in DMF, DMAC or NMP can also yield the bicyclic compounds 4-5.

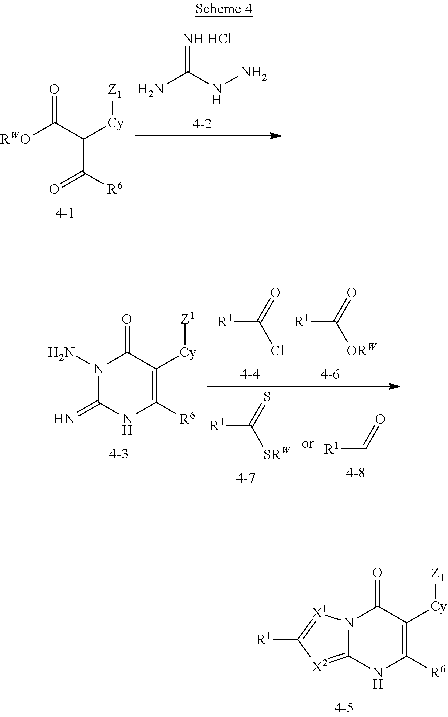

A series of intermediates of formula I-SM-B for preparations of formula I can be prepared by the method outlined in Scheme 5. Halogenation of the 3-keto esters 5-1 where R^{W} is C₁-C₆ alkyl (such as Me or Et) using a halogenation reagent such as SO₂Cl₂, NCS, Bromine, NBS or NIS in a suitable solvent such as dichloromethane can provide halogenated b-keto esters 5-2 (where W is Cl, Br or I) which can be conveniently transformed into the corresponding b-keto ester derivatives 5-4 by reaction with appropriate amines 5-3 (where Cy is a cyclic diamine with one N-protected group such as N-Boc, or N-Cbz) under basic conditions in the presence of a base such as TEA, Hunig's base or pyridine. The bicyclic compounds 5-6 can be obtained by condensation of 5-4 with triazole amines 5-5 where W² is halogen (e.g., Cl, Br, or I) or pseudohalogen (e.g., SMe or S(O)₂Me) under acidic conditions in the presence of an acid such as H₃PO₄. Suzuki coupling of the bicyclic compounds 5-6 with R¹W¹ 5-7 where W¹ is boronic acid or boronic ester groups can afford the corresponding bicyclic compounds 5-8 under standard Suzuki conditions (e.g., in the presence of a palladium catalyst, such as [1,1′-bis(diphenylphosphino) ferrocene]dichloropalladium (II) and a base, such as K₃PO₄). Alternatively, the R¹ substituted bicyclic compounds 5-8 where R¹ is a secondary or tertiary amino group can be obtained by replacement with amine derivative R¹—W¹ 5-7 (primary amine or secondary amine) under enhanced temperature or Buchwald coupling with the amine derivative R¹—W¹ 5-7 under Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, Cs₂CO₃, or K₂CO₃). Alkylation of the NH in the bicyclic compounds 5-8 to provide the intermediates of formula 5-10 can be achieved by reactions with a-halo-acetamides 5-9 where W³ is halogen (e.g., Cl, or Br) under basic conditions using a base such as TEA or Hunig's base in a suitable solvent such as DMF. Removal of the protecting group in 5-10 to provide the deprotected advanced intermediates I-SM-B can be achieved under acid conditions such as TFA, or HCl in dioxane when NP is N-Boc group or under hydrogenation in the presence of a Pd-catalyst such as Pd/C or Pd(OH)₂/C in a suitable solvent such as MeOH, EtOAc or THF when NP is N-Cbz.

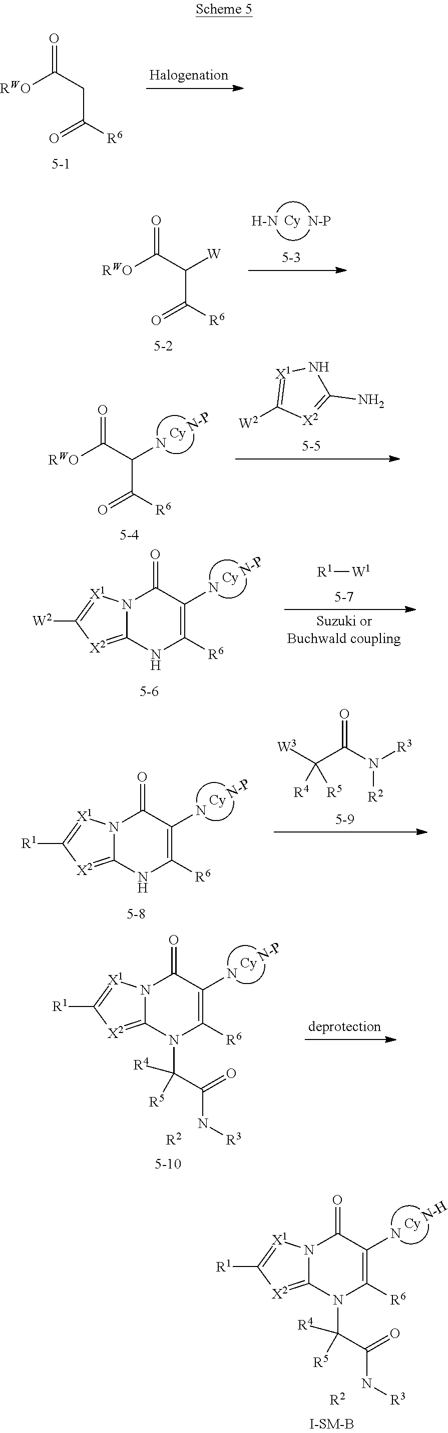

A series of intermediates of formula I-SM-A and I-SM-B for preparations of formula I can be prepared by the method outlined in Scheme 6. The bicyclic compounds 6-3 can be obtained by cyclization of the b-keto esters 6-1 where R^{W} is C₁-C₆ alkyl (such as Me or Et) with aminotriazoles 6-2 where X¹ and X² is independently N or aminopyrazoles 6-2 where X is CR⁷ under acidic conditions using an acid such as H₃PO₄ or PPA Alkylation of the NH in the bicyclic compounds 6-3 can be achieved by reactions of compounds 6-3 with a-haloacetamides 6-4 where W³ is halogen (e.g., Cl, or Br) under basic conditions using a base such as TEA or Hunig's base in a suitable solvent such as DMF to provide the intermediates 6-5 which can be halogenated by using a suitable halogenation reagent such as NCS, NBS or NIS in a suitable solvent such as acetonitrile to provide compounds 6-6 (W is Cl, Br or I). Suzuki coupling of the bicyclic compounds 6-6 with Z¹-Cy-W¹ 6-7 where W¹ is boronic acid or boronic ester groups can afford the corresponding intermediates I-SM-A under standard Suzuki conditions (e.g., in the presence of a palladium catalyst, such as [1,1′-bis(diphenylphosphino) ferrocene]dichloropalladium (II) and a base, such as K₃PO₄). Alternatively, treatment of the bicyclic compounds 6-6 with the diamines 6-8 under Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, Cs₂CO₃, or K₂CO₃) can provide compounds 6-9. Removal of the protecting group in 6-9 to provide the deprotected advanced intermediates I-SM-B can be achieved under acid conditions such as TFA, or HCl in dioxane when NP is N-Boc group or under hydrogenation conditions in the presence of a Pd-catalyst such as Pd/C or Pd(OH) 2/C in a suitable solvent such as MeOH, EtOAc or THF when NP is N-Cbz.

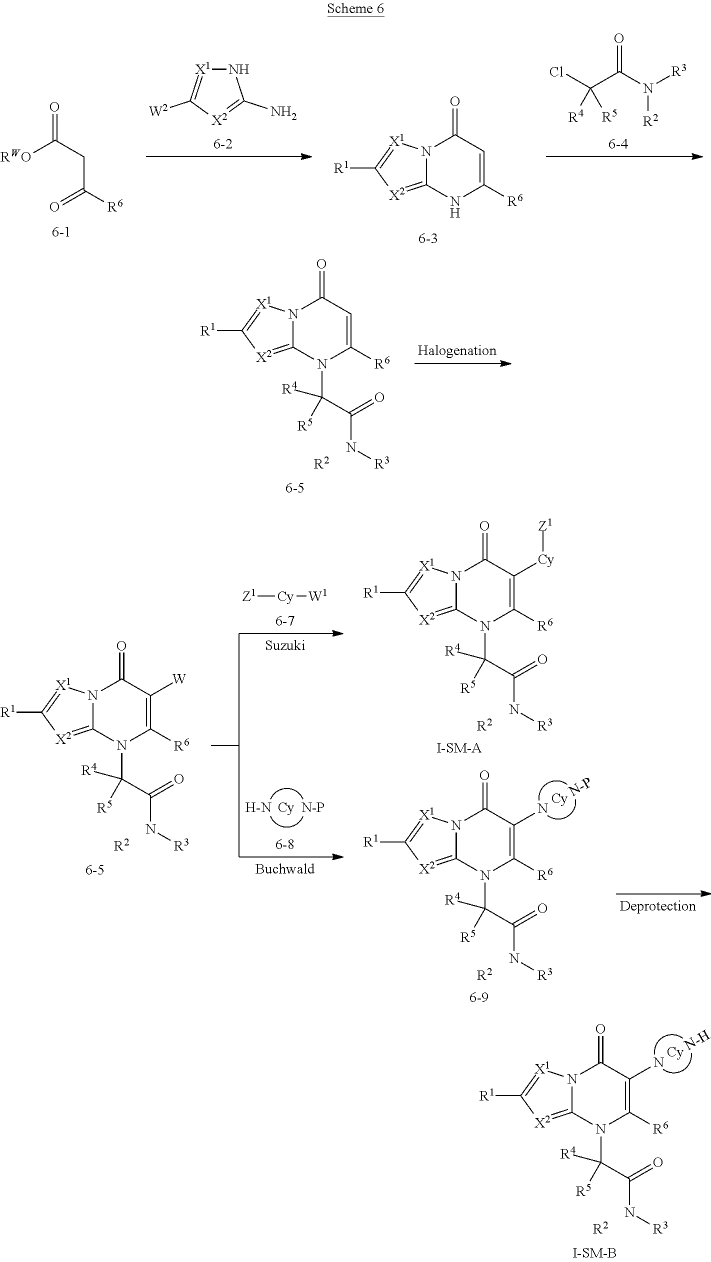

A series of compounds of formula VI, IX and X can be prepared by the method outlined in Scheme 7. When Z¹ group in compounds I-SM-A is a carbonate ester group, it can be saponified to carboxylic acids 7-1 under basic conditions in the presence of a base such as LiOH, NaOH or KOH. Coupling of the carboxylic acids 7-1 with amines R¹¹NH₂ 7-2 under standard amide coupling conditions (e.g., in the presence of a coupling reagent, such as BOP, PyBOP, HATU or HBTU, and a base, such as Et₃N or Hunig's base) can provide compounds formula VI. Similarly, with sulfonamides 7-3 can provide formula IX, and with sulfinamide 7-5 can provide compounds 7-6. Alternatively, treatment of the carboxylic acids 7-1 with the chlorine reagent such as oxalyl dichloride, thionyl chloride, POCl₃ or TCFH can produce the corresponding acid chlorides 7-4 which is subsequently coupled with the appropriate amines 7-2 to yield the corresponding compounds of formula VI, with sulfonamides 7-3 to formula IX, with sulfinamide 7-5 to compounds 7-6, and with sulfonimidamide 7-10 to the corresponding sulfonimidamides of formula X.

Sulfonamide 7-3 can be converted to sulfonamide 7-8 by treatment with TBSCl in the presence of a base such as triethylamine or Hunig's base. Sulfonimidoyl chloride 7-9 can be prepared from the sulfonamide 7-8 by treatment with Ph₃PCl₂ or SOCl₂ and Et₃N or Hunig's base. Reactions of the sulfonimidoyl chloride 7-9 with the amines R¹⁰NH₂ in the presence of a base such as triethylamine, Hunig's base or pyridine can afford sulfonimidamides 7-10.

Transformation of the sulfonides of formula IX into compounds of formula X (R¹⁰=H) can be achieved by treatment with sodium azide in the presence of an acid such as sulfuric acid or HCl. Alternatively, oxidation of the sulfinamides 7-6 with oxidative reagent such as PhI(OAc)₂ together with ammonium carbamate can provide sulfonimidamides of formula X (R¹⁰=H). In the other hand, the sulfonimidoyl chloride 7-7 can be prepared from the sulfonamide of formula IX by treatment with Ph₃PCl₂ or SOCl₂ and Et₃N or Hunig's base, or from the sulfinamides 7-6 by treatment with tert-butyl hypochlorite in tetrachloride. Reactions of the sulfonimidoyl chloride 7-7 with the amines R¹⁰NH₂ in the presence of a base such as triethylamine, Hunig's base or pyridine can afford sulfonimidamides of formula X. Alternatively, the sulfinamides 7-6 can be directly transformed into the sulfonimidamides of formula X by reaction with amines R¹⁰NH₂ in the presence of an oxidation reagent such as PhI(OAc)₂.

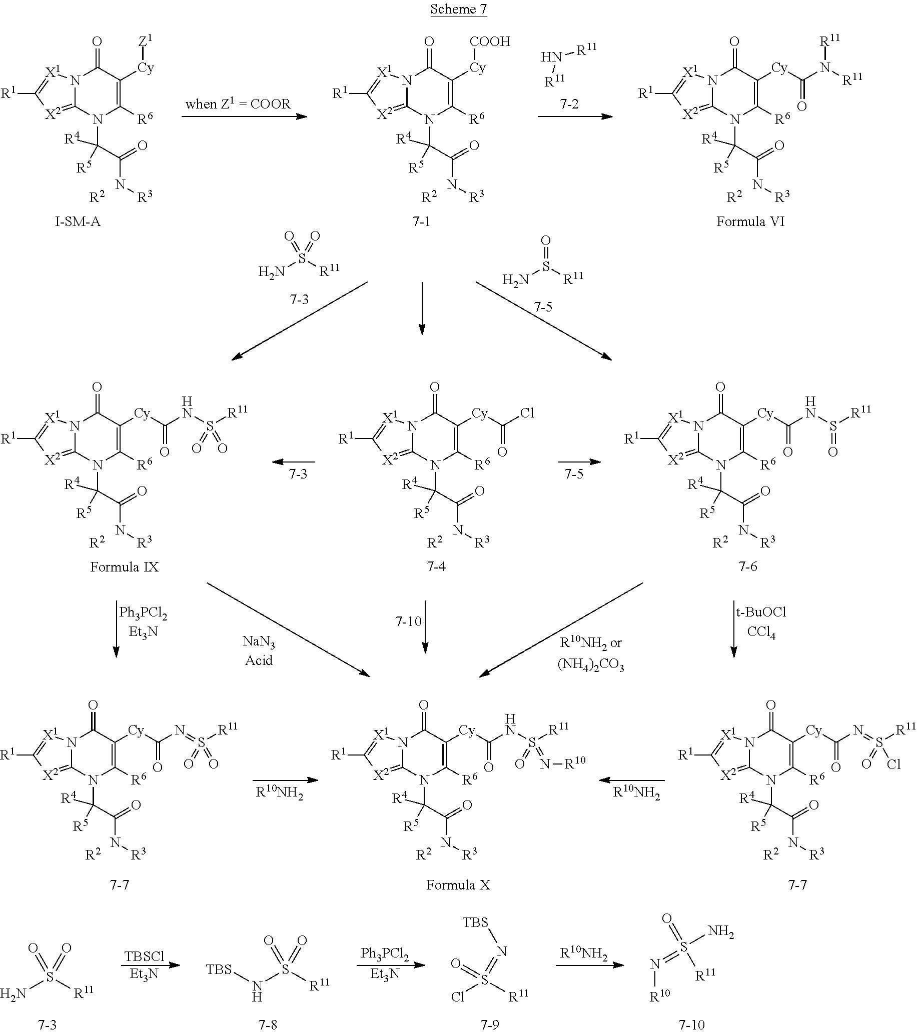

A series of compounds of formula VIIA, VIIB and VIII can be prepared by the method outlined in Scheme 8. When Z¹ group in compounds I-SM-A is a protected sulfonamide group, it can be deprotected to provide compounds 8-1 under acid conditions such as TFA or by hydrogenation in the presence of a Pd catalyst such as Pd/C or Pd(OH)₂/C. Reductive amination of the sulfonamides 8-1 with aldehydes R^{11A}CHO 8-4 can provide compounds of formula VIIA under standard reductive amination conditions (e.g., with a suitable reductive reagent such as NaBH(OAc)₃, NaBH₃CN or NaBH₄ in an appropriate solvent such as DCM, or DCE). Alternatively, alkylations of compounds 8-1 with R¹¹W 8-5 where W is halogen (e.g., Cl, Br or I) under basic conditions (e.g., in the presence of NaH, LiHMDS, NaHMDS etc.) or Mitsunobu reactions of compounds 8-1 with R¹¹W 8-5 where W is OH under Mitsunobo conditions (e.g., DEAD or DIAD with Ph₃P or Bu₃P) in a suitable solvent such as THF, diethyl ether DCM or toluene. Acylation of sulfonamides 8-1 with acyl chlorides 8-2 can provide compounds 8-3 which can be transformed into compounds of formula VIII by alkylation with halide R^{11A}CH₂Br or reductive amination with aldehyde R^{11A}CHO 8-4.

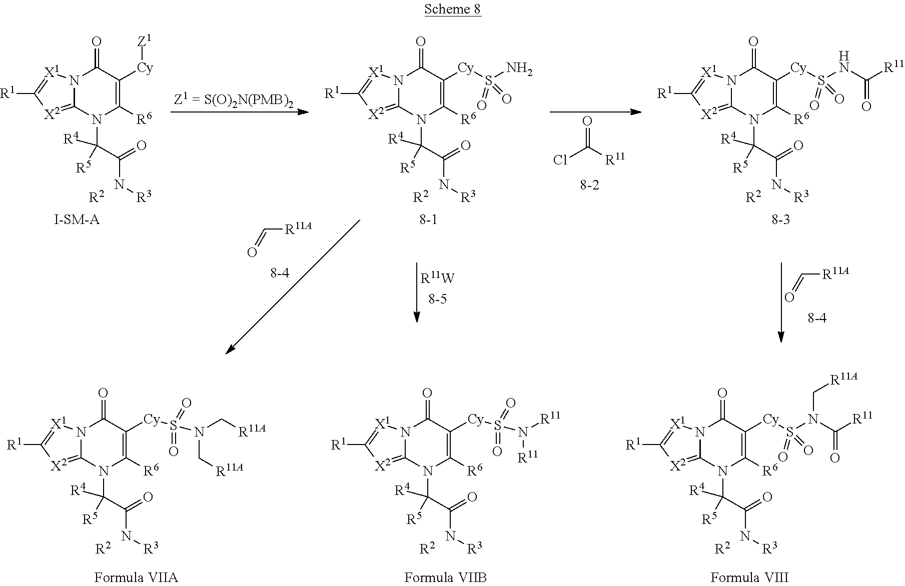

A series of compounds of formula II can be prepared by the method outlined in Scheme 9. When Z¹ group in compounds I-SM-A is an halogen such as Cl, Br or I, it can be directly transformed into compounds of formula II by Suzuki coupling with appropriate boronic acids or boronic esters 9-1 under standard Suzuki conditions (e.g., in the presence of a palladium catalyst, such as [1,1′-bis(diphenylphosphino) ferrocene]dichloropalladium (II) and a base, such as K₃PO₄). In the case of Z¹ is OMe or OBn group, it can be converted into the corresponding OH derivatives 9-2 which can be further transformed into the compounds 9-3 with a pseudohalogen (e.g., OTf or OMs) by treatment with Tf₂O or MsCl in the presence of a base such as Et₃N, Hunig's base or Pyridine. Suzuki coupling of the compounds 9-3 with appropriate boronic acids or boronic esters 9-1 under standard Suzuki conditions can afford the compounds of formula II.

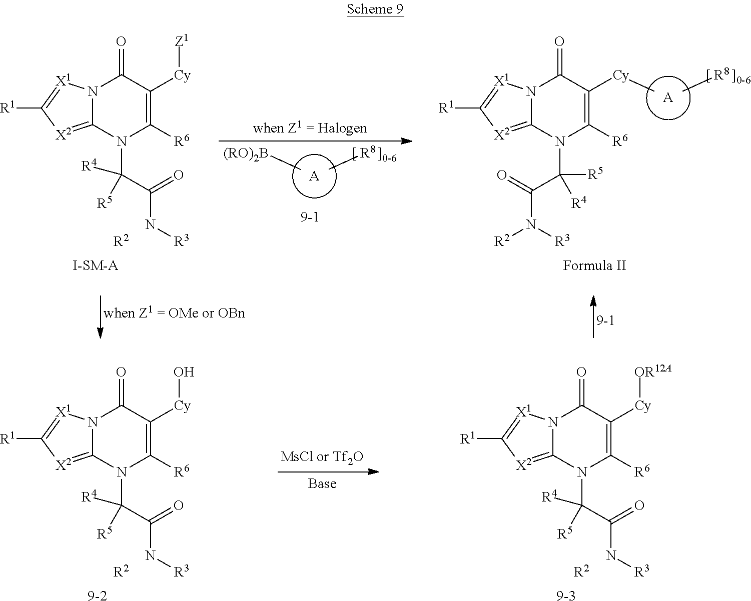

A series of compounds of formula II-B, III-B, VI-B and VII-B can be prepared by the method outlined in Scheme 10. Bicyclic derivatives II-B can be prepared by N-alkylation of I-SM-B with suitable compounds 10-1 where W¹ is halogen (e.g., Cl, Br, or I) or pseudohalogen (e.g., OTf or OMs) under alkylation conditions (e.g., in the presence of a base, such as Hunig's base, NaH, t-BuOK, t-BuONa, Cs₂CO₃, or K₂CO₃). Alternatively, bicyclic derivatives II-B can be obtained by coupling I-SM-B with compounds 10-1 where ring A is aryl, heteroaryl or alkene and W¹ is a halogen (e.g., Br, or I) or pseudohalogen (e.g., OTf) under standard Buchwald-Hartwig amination conditions (e.g., in the presence of a palladium catalyst, such as XPhos Pd G3, and a base, such as Cs₂CO₃ or K₃PO₄).

Compounds III-B can be obtained by amide formation reactions of I-SM-B and acids 10-2 where W² is OH under standard amide coupling conditions (e.g., in the presence of a coupling reagent, such as BOP, PyBOP, HATU or HBTU, and a base, such as Et₃N or Hunig's base). Alternatively, compounds III-B can be prepared using acid chlorides 10-2 (where W² is Cl) in a suitable solvent such dichloromethane in the presence of a base such as TEA or Hunig's base. Reactions of I-SM-B with isocyanates R¹¹N═C═O 10-3 can provide VI-B and with sulfamoyl chlorides 10-4 in the presence of a base such as TEA can yield VII-B.

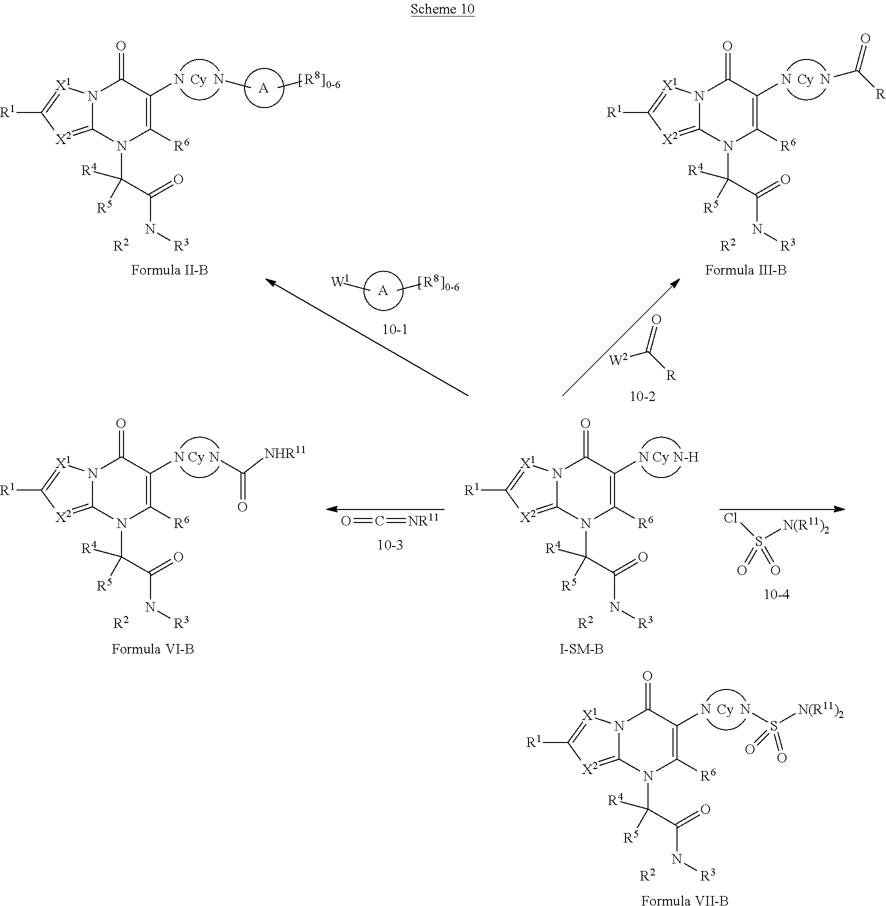

A series of the carbamoylsulfonamide derivatives of formula IX—B can be prepared by the method outlined in Scheme 11. Primary sulfonamides 11-1 can react with carbonochloridate 11-2 where W¹ is aryl or alkyl group (e.g., ethyl or phenyl) in the presence of a base such as Et₃N or Hunig's base to yield the intermediates 11-3 which can react with I-SM-B in the presence of a base such as DMAP or TEA to provide the desired carbamoylsulfonamide derivatives 11-4 which can be transformed into compounds of formula IX—B by alkylation with halide R^{11A}CH₂Br or reductive amination with aldehyde R^{11A}CHO 11-5.

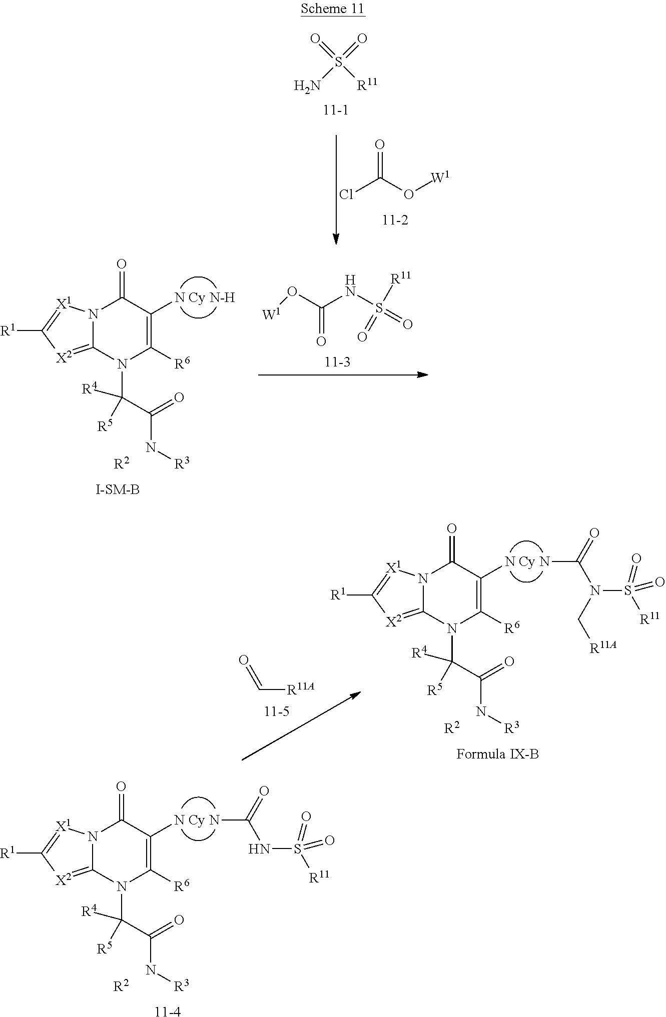

A series of the amidine compounds of formula IV-B can prepared by the method outlined in Scheme 12. Amides 12-3 can be prepared from the acids (W is OH) or acid chloride (W is Cl) and the primary amines R¹⁰—NH₂ 12-2 under standard amide coupling conditions (e.g., in the presence of a coupling reagent, such as BOP, PyBOP, HATU or HBTU, and a base, such as Et₃N or Hunig's base). Treatment of the amides 12-3 with oxalyl chloride or POCl₃ in a suitable solvent such as dichloromethane in the presence of a base such as 2,6-lutidine can provide the corresponding chlorides 12-4. Reactions of the intermediate I-SM-B with the chlorides 12-4 in the presence of a base such as Et₃N or Hunig's base can provide the amidine compounds of formula IV-B.

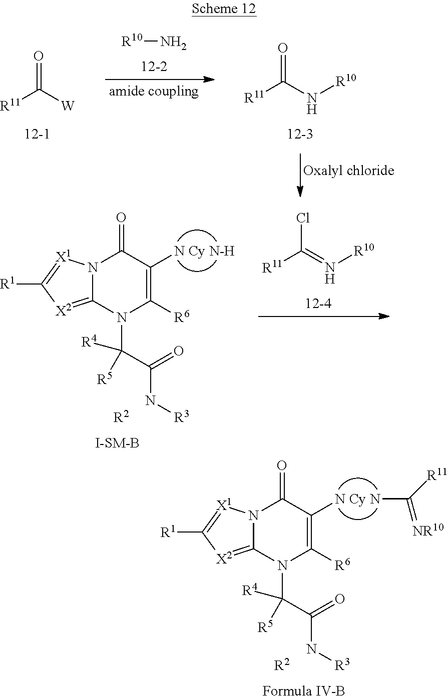

A series of acylsulfonimidamide derivatives of formula X—B can be prepared by the method outlined in Scheme 13. Reaction of the sulfonyl chlorides 13-1 with amines 13-2 in a suitable solvent such as dichloromethane in the presence of a base such as Hunig's base can yield sulfonamides 13-3 which can be transformed into sulfonimidamides 13-4 by treatment with (Ph)₃PCl₂ in a suitable dry solvent such as dichloromethane in the presence of a base such as TEA followed by addition of ammonia (NH₃) (g). Acylation of the sulfonimidamides 13-4 with ethyl chloroformate 13-5 can yield compounds 13-6. The desired acylsulfonimidamide derivatives of formula X—B can be prepared by reacting the advanced intermediates I-SM-B with compounds 13-6 in a suitable solvent in the presence of a base such as TEA or Hunig's base.

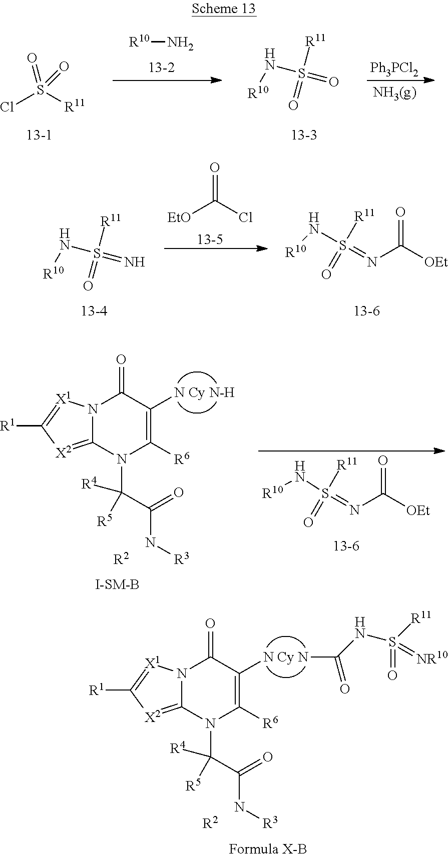

A series of carboximidamide derivatives of formula V-B can be prepared by the method outlined in Scheme 14. Conversions of the amines R¹⁰—NH₂ 14-1 to isothiocyanates 14-2 can be achieved by reactions with (Me₄N)SCF₃ in a suitable solvent such as dichloromethane in the presence of a base such as TEA. Formation of the thioureas 14-4 can be carried out by treating the isothocyanates 14-2 with amines R¹¹—NH₂ 14-3 with or without a solvent. The carbodiimide derivatives 14-5 can be synthesized by treating the thiourea 14-4 with iodine in the presence of triphenylphosphine in a suitable solvent such as dichloromethane. Reactions of the advanced intermediates I-SM-B with carbodiimides 14-5 with in a suitable solvent such as DMF can provide the desired carboximidamide derivatives V-B.

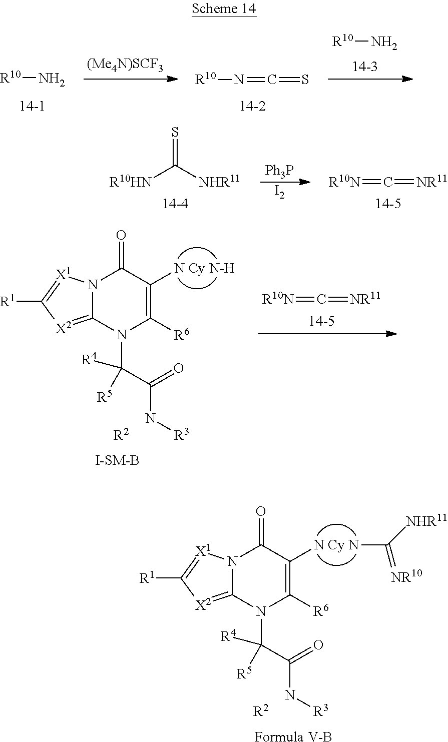

A series of compounds of formula VIII-B can be prepared by the method outlined in Scheme 15. Reactions of sulfurisocyanatidic chloride 15-1 with 4-nitrophenol 15-2 can provide 4-nitrophenyl sulfamate 15-3 which can be coupled with the advanced intermediates I-SM-B to yield the sulfone-urea derivatives 15-4. The sulfone-ureas 15-4 can be transformed into compounds of formula VIII-B by amide coupling with acids R¹¹COOH 15-5 under amide coupling conditions (e.g., in the presence of a coupling reagent, such as EDCI and a base, such as Et₃N or Hunig's base) or by acylation with acid chlorides R¹¹C(O)Cl 15-6 in the presence of a base such as TEA or Hunig's base. Alternatively, treatment of sulfurisocyanatidic chloride 15-1 with the acids R¹¹COOH 15-5 in a suitable solvent such as toluene can provide acylsulfamoyl chloride derivatives 15-7 which can couple with the advanced intermediates I-SM-B to afford the compounds of formula VIII-B in the presence of a base such as TEA or Hunig's base.

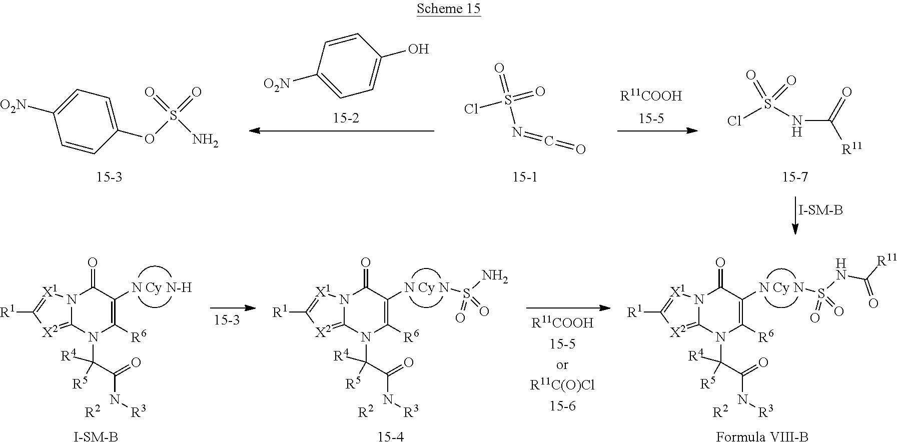

### Abbreviations

| | |
|---|---|
| (Me₄N)SCF₃ | Tetramethylammonium Trifluoromethanethiolate |
| AcNHOH | Acetohydroxamic acid |
| AcOH | Acetic acid |
| ATP | Adenosine triphosphate |
| BnOH | Benzyl alcohol |
| Boc | t-Butyloxy carbonyl |
| ADP | Adenosine diphosphate |
| aq. | Aqueous |
| BOP | (Benzotriazol-1-yloxy)tris(dimethylamino)phosphonium |
| | hexafluorophosphate |
| BPO | Benzoyl peroxide |
| BrettPhos Pd G3 | [(2-Di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| brine | Saturated solution of sodium chloride |
| BSA | Bovine albumin |
| Bu₃P | Tributylphosphine |
| Cbz | Benzoxycarbonyl |
| DCE | 1,2-Dichloroethylene |
| Cs₂CO₃ | Cesium carbonate |
| DCM | Dichloromethane |
| DEAD | Diethyl azodicarboxylate |
| DIAD | Diisopropyl Azodicarboxylate |
| DIEA | N,N-diisopropylethylamine |
| DIPEA | N,N-Diisopropylethylamine |
| DMF-DMA | N,N-dimethyl formamide dimethyl acctel |
| DMAC | Dimethylacetamide |
| DMAP | 4-Dimethylaminopyridine |
| DME | Dimethyl ether |
| DMF | N,N-dimethylformamide |
| DMSO | Dimethylsulfoxide |
| DPPA | Diphenylphosphoryl azide |
| EDCI | N-(3-Dimethylaminopropyl)-N′-ethylcarbodiimide hydrochloride |
| Et | Ethyl |
| Et₂O | Ethyl ether |
| Et₃N/TEA | Triethylamine |
| EtOAc | Ethyl acetate |
| EtOH | Ethanol |
| ¹H NMR | Hydrogen-1 nuclear magnetic resonance spectroscopy |
| FA | Formic acid |
| HATU | 2-(7-Azabenzotriazol-1-yl)-N,N,N′,N′-tetramethyluronium |
| | hexafluorophosphate |
| HBTU | O-(Benzotriazol-1-yl)-N,N,N′,N′-tetramethyluronium |
| | hexafluorophosphate |
| HEPES | N-2-Hydroxyethylpiperazine-N-ethane-sulphonicacid |
| HOBt | 1-Hydroxybenzotriazol |
| HNMe₂ | Dimethylamine |
| KOAc | Potassium Acetate |
| KOH | Potassium hydroxide |
| LCMS | Liquid chromatography-mass spectrometry |
| LDA | Lithium diisopropylamide |
| LiHMDS | Lithium Hexamethyldisilazide |
| m-CPBA | m-Chloroperbenzoic Acid |
| Me | Methyl |
| MeCN/ACN | Acetonitrile |
| MeOH | Methanol |
| MeI | Methyl Iodide |
| MeNH₂ | Methylamine |
| MsCl | Methanesulfonyl chloride |
| Na₂CO₃ | Sodium carbonate |
| Na₂SO₄ | Sodium sulfate |
| NaBH₄ | Sodium borohydride |
| NaHMDS | Sodium bis(trimethylsilyl)amide |
| NaOEt | Sodium ethoxide |
| NaOMe | Sodium methoxide |
| NaSEt | Sodium ethanethiolate |
| NBS | N-Bromosuccinimide |
| nBu₄NOH | Tetrabutylammonium hydroxide |
| NCS | N-chlorosuccinimide |
| NIS | N-Iodosuccinimide |
| NMI | 1-Methylimidazole |
| NMP | N-Methylpyrrolidone |
| NMI | 1-Methylimidazole |
| Pd(dppf)Cl₂ | [1,1′-Bis(diphenylphosphino)ferrocene]dichloropalladium(II) |
| Pd(dppf)Cl₂.DCM | 1,1′-Bis(diphenylphosphino)ferrocene-palladium(II)dichloride |
| | dichloromethane complex |
| Pd(OAc)₂ | Palladium (II) Acetate |
| Pd₂(dba)₃ | Bis(dibenzylideneacetone)palladium(0) |
| PdCl₂(PPh₃)₂ | Bis(triphenylphosphine) Palladium (II) Chloride |
| PE | Petroleum ether |
| Ph₃P | Triphenylphosphine |
| Ph₃PCl₂ | Dichlorotriphenylphosphorane |
| PhI(OAc)₂ | (Diacetoxyiodo)benzene |
| PMBCl | 4-Methoxybenzylchloride |
| PPA | Polyphosphoric acid |
| prep-HPLC | Preparative High-Performance Liquid Chromatography |
| Prep-TLC | Preparative Thin Layer chromatography |
| PyBOP | (Benzotriazol-1-yloxy)tripyrrolidinophosphonium hexafluorophosphate |
| r.t. | Room temperature |
| RuPhos Pd G3 | (2-Dicyclohexylphosphino-2′,6′-diisopropoxy-1,1′-biphenyl)[2-(2′- |
| | amino-1,1′-biphenyl)]palladium(II) methanesulfonate |
| SEMCl | 2-Chloromethyl 2-(trimethylsilyl)ethyl ether |
| T₃P | 1-Propanephosphonic anhydride |
| TBAF | Tetrabutylammonium fluoride |
| TBSCl | tert-Butyldimethylsilyl chlorid |
| t-BuOK | Potassium tert-butoxide |
| t-BuONa | Sodium tert-butoxide |
| t-BuXphos Pd G3 | [(2-Di-tert-butylphosphino-2′,4′,6′-triisopropyl-1,1′-biphenyl)-2-(2′- |
| | amino-1,1′-biphenyl)] palladium(II) methanesulfonate |
| TCFH | Chloro-N,N,N′,N′-tetramethylformamidinium hexafluorophosphate |
| TEA | Triethylamine |
| Tf₂O | Trifluoromethanesulfonic anhydride |
| TFA | Trifluoroacetic acid |
| THF | Tetrahydrofuran |
| TMSCN | Trimethylsilyl cyanide |
| Xant-Phos | 4,5-Bis(diphenylphosphino)-9,9-dimethylxanthene |
| XantPhos Pd G3 | [(4,5-Bis(diphenylphosphino)-9,9-dimethylxanthene)-2-(2′-amino-1,1′- |
| | biphenyl)]palladium(II) methanesulfonate |
| Xphos | 2-Dicyclohexylphosphino-2′,4′,6′-triisopropylbiphenyl |
| XPhos Pd G3 | Methanesulfonato(2-dicyclohexylphosphino-2′,4′,6′-tri-i-propyl-1,1′- |
| | biphenyl)(2′-amino-1,1′-biphenyl-2-yl)palladium(II) |

1M or 1N = 1 mol/L, 2M or 2N = 2 mol/L etc.

### EXAMPLES

### Example 1: N-(2-Chloro-4-(trifluoromethyl)phenyl)-2-(2-(3,6-dihydro-2H-pyran-4-yl)-5-ethyl-7-oxo-6-(4-(4-(trifluoromethyl)-1H-imidazole-2-carbonyl)piperazin-1-yl)-[1,2,4]triazolo[1,5-a]pyrimidin-4(7H)-yl)acetamide

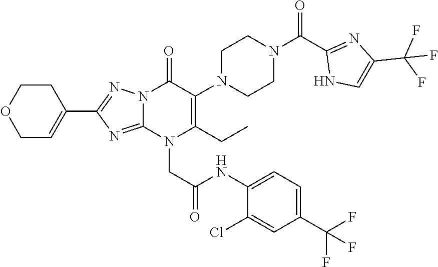 [SMILES: CCc1c(N2CCN(C(=O)c3nc(C(F)(F)F)c[nH]3)CC2)c(=O)n2nc(C3=CCOCC3)nc2n1CC(=O)Nc1ccc(C(F)(F)F)cc1Cl]

Method A: A mixture of 4-(trifluoromethyl)-1H-imidazole-2-carboxylic acid (15.91 mg, 0.088 mmol), HATU (41.83 mg, 0.11 mmol) and DIEA (23.26 mg, 0.18 mmol), INT B1 (50 mg, 0.088 mmol) in dry DMF (2 mL) was stirred at 20° C. for 1 h. under N₂. Then the reaction mixture was filtered and concentrated under reduced pressure. The residue was purified by Prep-HPLC on a C18 column eluting with MeOH/H₂O (50%-95%, with 0.1% TFA) to afford the title compound (18 mg, 28% yield) as a white solid. ¹H NMR (400 MHZ, DMSO-d₆) δ ppm 13.60 (s, 1H), 10.37 (s, 1H), 8.03 (dd, J=25.7, 8.5 Hz, 1H), 7.95 (dd, J=8.7, 2.9 Hz, 2H), 7.75-7.69 (m, 1H), 6.85-6.57 (m, 1H), 5.43-5.14 (m, 3H), 4.61-4.46 (m, 1H), 4.26 (d, J=8.9, 2.9 Hz, 2H), 3.84-3.75 (m, 2H), 3.46 (ddt, J=34.7, 24.5, 11.2 Hz, 4H), 3.01 (p, J=6.4 Hz, 3H), 2.88-2.63 (m, 3H), 1.19 (dt, J=11.2, 7.4 Hz, 3H). LCMS calc. for C₃₀H₂₉ClF₆N₉O₄ [M+H]⁺: m/z=728.2; Found: 727.7.

### Example 2:2-(6-(4-(3-Aminothieno[3,2-b]pyridine-2-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

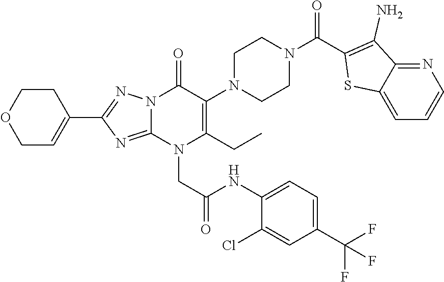 [SMILES: CCc1c(N2CCN(C(=O)c3sc4cccnc4c3N)CC2)c(=O)n2nc(C3=CCOCC3)nc2n1CC(=O)Nc1ccc(C(F)(F)F)cc1Cl]

This compound was prepared by procedures analogous to those described for method A using INT B1 and 3-aminothieno[3,2-b]pyridine-2-carboxylic acid to afford the title product as a white solid. ¹H NMR (400 MHZ, DMSO-d₆) δ ppm 10.36 (s, 1H), 8.66 (dd, J=4.5, 1.4 Hz, 1H), 8.38 (dd, J=8.2, 1.4 Hz, 1H), 8.06 (d, J=8.5 Hz, 1H), 7.97 (d, J=1.5 Hz, 1H), 7.72 (d, J=8.5 Hz, 1H), 7.48 (dd, J=8.2, 4.5 Hz, 1H), 6.83 (s, 1H), 6.17 (s, 2H), 5.33 (s, 2H), 4.26 (d, J=10.7 Hz, 4H), 3.80 (t, J=5.4 Hz, 2H), 3.49 (t, J=10.5 Hz, 2H), 3.25 (d, J=11.5 Hz, 4H), 3.01 (d, J=7.5 Hz, 2H), 2.77 (d, J=11.3 Hz, 2H), 1.21 (t, J=7.5 Hz, 3H). LCMS calc. for C₃₃H₃₂ClF₃N₉O₄S [M+H]⁺: m/z=742.2; Found: 741.8.

### Example 3: N-(2-Chloro-4-(trifluoromethyl)phenyl)-2-(2-(3,6-dihydro-2H-pyran-4-yl)-5-ethyl-6-(4-(4-hydroxy-1H-pyrazolo[3,4-b]pyridine-5-carbonyl)piperazin-1-yl)-7-oxo-[1,2,4]triazolo[1,5-a]pyrimidin-4(7H)-yl)acetamide

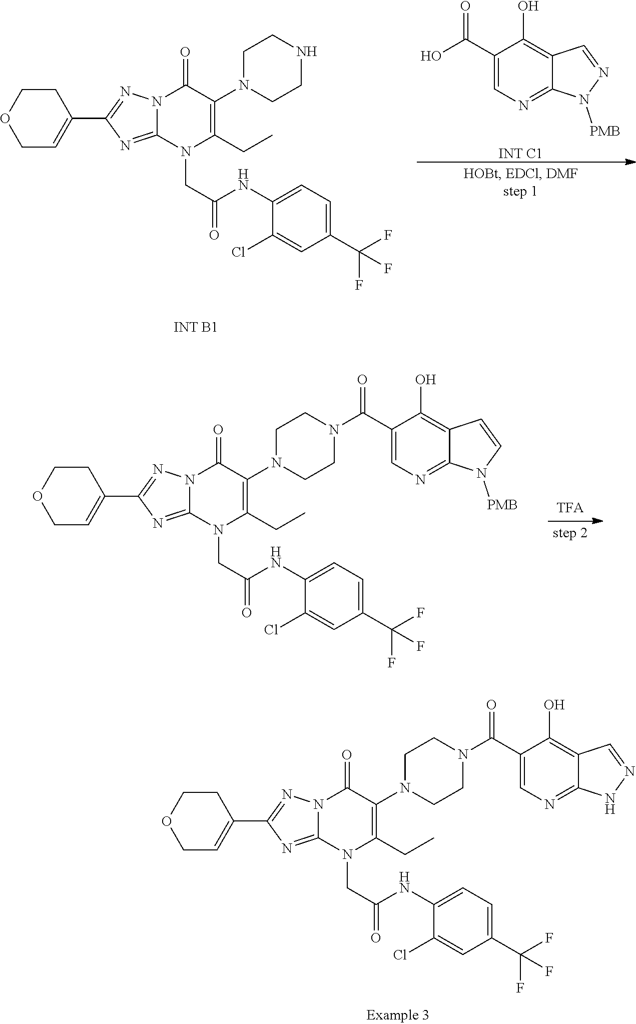

### Step 1: N-(2-chloro-4-(trifluoromethyl)phenyl)-2-(2-(3,6-dihydro-2H-pyran-4-yl)-5-ethyl-6-(4-(4-hydroxy-1-(4-methoxybenzyl)-1H-pyrazolo[3,4-b]pyridine-5-carbonyl)piperazin-1-yl)-7-oxo-[1,2,4]triazolo[1,5-a]pyrimidin-4(7H)-yl)acetamide

To a mixture of INT C1 (30 mg, 0.10 mmol), INT B1 (68 mg, 0.20 mmol), and HOBt (16 mg, 0.12 mmol) in DMF (2 mL) was added EDCI (27 mg, 0.14 mmol) at 0° C. under N₂ atmosphere. The resulting mixture was stirred at r.t. for 16 h., and purified by Prep-HPLC eluting with MeCN/H₂O (45%-60%, with 0.1% NH₄HCO₃) to give the title compound (34 mg, 40% yield) as a white solid. LCMS calc. for C₄₀H₃₉ClF₃N₁₀O₆ [M+H]⁺: m/z=847.3; Found: 847.3.

### Step 2: N-(2-chloro-4-(trifluoromethyl)phenyl)-2-(2-(3,6-dihydro-2H-pyran-4-yl)-5-ethyl-6-(4-(4-hydroxy-1H-pyrazolo[3,4-b]pyridine-5-carbonyl)piperazin-1-yl)-7-oxo-[1,2,4]triazolo[1,5-a]pyrimidin-4(7H)-yl)acetamide

A solution of N-(2-chloro-4-(trifluoromethyl)phenyl)-2-(2-(3,6-dihydro-2H-pyran-4-yl)-5-ethyl-6-(4-(4-hydroxy-1-(4-methoxybenzyl)-1H-pyrazolo[3,4-b]pyridine-5-carbonyl)piperazin-1-yl)-7-oxo-[1,2,4]triazolo[1,5-a]pyrimidin-4(7H)-yl)acetamide (30 mg, 0.035 mmol) in TFA (2 mL) under N₂ was stirred at 80° C. for 2 h. The mixture was concentrated under reduced pressure and purified by Prep-HPLC on a C18 column eluting with MeCN/H₂O (45%-60%, with 0.1% NH₄HCO₃) to give the title compound (10.3 mg, 40% yield) as a white solid. ¹H NMR (400 MHZ, CDCl₃) δ ppm 8.25 (s, 1H), 8.15 (d, J=8.6 Hz, 1H), 7.97 (s, 1H), 7.70 (s, 2H), 7.51 (d, J=8.4 Hz, 1H), 6.73 (d, J=140.8 Hz, 1H), 5.26 (d, J=54.2 Hz, 2H), 4.31 (s, 2H), 3.88 (s, 2H), 3.67 (s, 4H), 3.06 (d, J=7.3 Hz, 2H), 2.63 (s, 4H), 1.28 (t, J=7.1 Hz, 3H). LCMS calc. for C₃₂H₃₁ClF₃N₁₀O₅ [M+H]⁺: m/z=727.2; Found: 727.2.

### Example 4: N-(2-Chloro-4-(trifluoromethyl)phenyl)-2-(2-(3,6-dihydro-2H-pyran-4-yl)-5-ethyl-6-(4-(4-hydroxy-6-methyl-2-oxo-2H-pyran-3-carbonyl)piperazin-1-yl)-7-oxo-[1,2,4]triazolo[1,5-a]pyrimidin-4(7H)-yl)acetamide

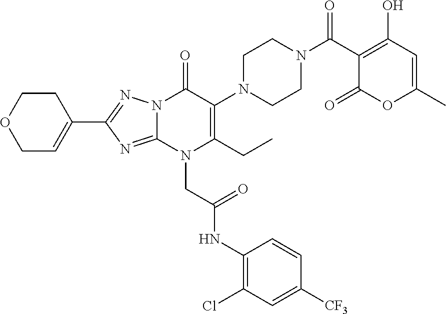 [SMILES: CCc1c(N2CCN(C(=O)c3c(O)cc(C)oc3=O)CC2)c(=O)n2nc(C3=CCOCC3)nc2n1CC(=O)Nc1ccc(C(F)(F)F)cc1Cl]

To a solution of INT C2 (20 mg, 0.12 mmol), INT B1 (80 mg, 0.14 mmol) and HATU (68 mg, 0.18 mmol) in DMF (2 mL) was added DIPEA (46 mg, 0.35 mmol) at 0° C. under N₂. The resulting mixture was stirred at r.t. for 2 h. The mixture was concentrated. The residue was purified by Prep-HPLC on a C18 column eluting with MeCN/H₂O (45%-60% with 0.1% NH₄HCO₃) to afford the title compound (10.14 g, 12.0% yield) as a white solid. ¹H NMR (400 MHZ, DMSO-d₆) δ ppm 12.18 (s, 1H), 10.36 (s, 1H), 8.06 (d, J=8.4 Hz, 1H), 7.96 (d, J=1.7 Hz, 1H), 7.72 (s, 1H), 7.09 (s, 1H), 6.83 (s, 1H), 5.75 (s, 1H), 5.31 (s, 2H), 4.25 (d, J=2.5 Hz, 2H), 3.80 (t, J=5.4 Hz, 2H), 3.41 (s, 5H), 2.98 (d, J=7.6 Hz, 4H), 2.69-2.62 (m, 2H), 2.09 (s, 3H), 1.19 (t, J=7.5 Hz, 3H). LCMS calc. for C₃₂H₃₂ClF₃N₇O₇ [M+H]⁺: m/z=718.2; Found: 718.2.

### Example 5: 2-(2-(3,6-Dihydro-2H-pyran-4-yl)-6-(4-(N′-(3-(dimethylamino) propyl)-N-ethylcarbamimidoyl)piperazin-1-yl)-5-ethyl-7-oxo-[1,2,4]triazolo[1,5-a]pyrimidin-4(7H)-yl)-N-(2-methyl-4-(trifluoromethyl)phenyl)acetamide

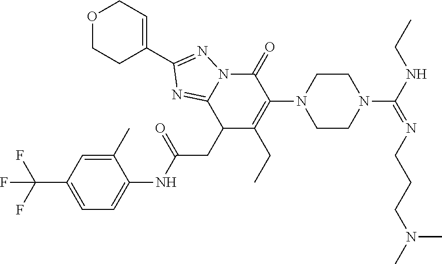 [SMILES: CCN/C(=N/CCCN(C)C)N1CCN(C2=C(CC)C(CC(=O)Nc3ccc(C(F)(F)F)cc3C)c3nc(C4=CCOCC4)nn3C2=O)CC1]

A mixture of INT B2 (170 mg, 0.31 mmol), EDCI (118.8 mg, 0.62 mmol), HOBt (83.77 mg, 0.62 mmol) and DIEA (200 mg, 1.55 mmol) in DMF (2 mL) was stirred at r.t. overnight. The crude product was purified by Prep-HPLC on a C18 column eluting with MeCN/H₂O (45%-60%, with 0.1% NH₄HCO₃) to afford the title compound (10 mg, 4.6% yield) as a white solid. ¹H NMR (400 MHz, MeOH-d₄) δ ppm 7.63 (d, J=8.3, 1H), 7.54 (s, 1H), 7.46 (d, J=8.6, 1H), 6.96 (s, 1H), 5.31 (s, 1H), 4.33 (d, J=2.5, 2H), 3.90 (t, J=5.4, 2H), 3.83-3.64 (m, 4H), 3.44-3.32 (m, 6H), 3.12 (dt, J=14.9, 4.5, 2H), 2.89 (d, J=11.7, 2H), 2.64 (s, 2H), 2.43 (t, J=6.5, 2H), 2.37 (s, 3H), 2.28 (s, 6H), 1.91-1.76 (m, 2H), 1.42-1.21 (m, 6H). LCMS calc. for C₃₄H₄₈F₃N₁₀O₃ [M+H]⁺: m/z=701.4; Found: 701.3.

### Example 6: 2-(6-(4-(3-Aminothieno[2,3-c]pyridine-2-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

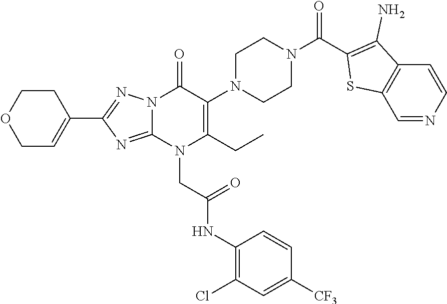 [SMILES: CCc1c(N2CCN(C(=O)c3sc4cnccc4c3N)CC2)c(=O)n2nc(C3=CCOCC3)nc2n1CC(=O)Nc1ccc(C(F)(F)F)cc1Cl]

This compound was prepared by procedures analogous to those described for Example 4 using INT B1 and INT C3 to afford the title product as a white solid. ¹H NMR (400 MHZ, DMSO-d₆) δ ppm 10.37 (s, 1H), 9.12 (s, 1H), 8.49 (d, J=5.5 Hz, 1H), 8.07 (d, J=8.5 Hz, 1H), 8.00 (d, J=5.5 Hz, 1H), 7.87 (s, 1H), 7.63 (s, 1H), 6.83 (s, 1H), 6.41 (s, 2H), 5.23 (s, 2H), 4.25 (d, J=2.7 Hz, 4H), 3.79 (d, J=5.4 Hz, 2H), 3.49 (s, 2H), 3.27 (d, J=13.7 Hz, 4H), 3.00 (d, J=6.9 Hz, 2H), 2.76 (d, J=11.4 Hz, 2H), 1.21 (t, J=7.5 Hz, 3H). LCMS calc. for C₃₃H₃₂ClF₃N₉O₄S [M+H]⁺: m/z=742.2; Found: 741.8.

### Example 7: 2-(6-(4-(5H-Pyrrolo[3,2-d]pyrimidine-4-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

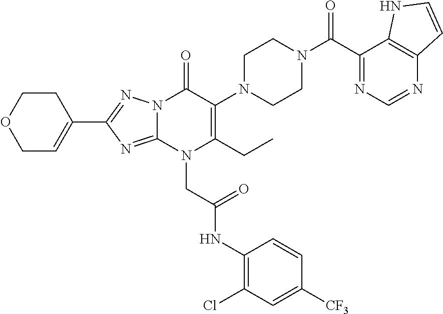 [SMILES: CCc1c(N2CCN(C(=O)c3ncnc4cc[nH]c34)CC2)c(=O)n2nc(C3=CCOCC3)nc2n1CC(=O)Nc1ccc(C(F)(F)F)cc1Cl]

This compound was prepared by procedures analogous to those described for method A using INT B1 and 5H-pyrrolo[3,2-d]pyrimidine-4-carboxylic acid to afford the title product as a white solid. ¹H NMR (400 MHZ, DMSO-d₆)₈ ppm 12.11 (s, 1H), 10.35 (s, 1H), 8.87 (s, 1H), 8.06 (d, J=8.5 Hz, 1H), 7.97-7.93 (m, 2H), 7.71 (d, J=8.4 Hz, 1H), 6.83 (s, 1H), 6.70 (d, J=3.2 Hz, 1H), 5.32 (s, 2H), 4.65 (d, J=12.5 Hz, 1H), 4.25 (d, J=2.9 Hz, 2H), 4.01 (d, J=12.5 Hz, 1H), 3.80 (t, J=5.6 Hz, 2H), 3.54 (d, J=33.8 Hz, 3H), 3.20-2.73 (m, 5H), 2.69-2.65 (m, 1H), 1.20 (t, J=7.4 Hz, 3H). LCMS calc. for C₃₂H₃₁ClF₃N₁₀O₄ [M+H]⁺: m/z=711.2; Found: 711.2.

### Example 8: N-(2-Chloro-4-(trifluoromethyl)phenyl)-2-(6-(4-(5-(difluoromethyl)-1,3,4-thiadiazol-2-yl)piperazin-1-yl)-2-(3,6-dihydro-2H-pyran-4-yl)-5-ethyl-7-oxo-[1,2,4]triazolo[1,5-a]pyrimidin-4(7H)-yl)acetamide

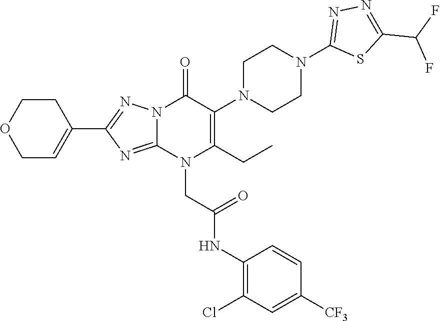 [SMILES: CCc1c(N2CCN(c3nnc(C(F)F)s3)CC2)c(=O)n2nc(C3=CCOCC3)nc2n1CC(=O)Nc1ccc(C(F)(F)F)cc1Cl]

A mixture of INT B1 (40 mg, 0.07 mmol), 2-bromo-5-(difluoromethyl)-1,3,4-thiadiazole (61 mg, 0.28 mmol) and Cs₂CO₃ (69 mg, 0.21 mmol) in DMF (1 mL) was stirred at 60° C. for 1 h. After completion of the reaction, the mixture was purified by Prep-HPLC on a C18 column eluting with MeCN/H₂O (45%-60%, with 0.05% FA) to afford the title compound (9.48 mg, 19% yield) as a white solid. ¹H NMR (400 MHZ, DMSO-d₆) δ ppm 10.37 (s, 1H), 8.06 (d, J=8.5 Hz, 1H), 7.97 (d, J=1.6 Hz, 1H), 7.72 (dd, J=8.6, 1.6 Hz, 1H), 7.36 (t, J=53.3 Hz, 1H), 6.84 (s, 1H), 5.33 (s, 2H), 4.26 (d, J=2.5 Hz, 2H), 3.94 (d, J=12.1 Hz, 2H), 3.80 (t, J=5.4 Hz, 2H), 3.64 (dd, J=11.8, 9.2 Hz, 2H), 3.43 (dd, J=12.1, 9.5 Hz, 2H), 3.00 (q, J=7.3 Hz, 2H), 2.82 (d, J=11.2 Hz, 2H), 2.51 (s, 2H), 1.26-1.16 (m, 3H). LCMS calc. for C₂₈H₂₈ClF₅N₉O₃S [M+H]⁺: m/z=700.1; Found: 700.1. Example 9: N-(2-Chloro-4-(trifluoromethyl)phenyl)-2-(2-(3,6-dihydro-2H-pyran-4-yl)-5-ethyl-6-(4-(5-(5-fluoro-2-methoxypyridin-4-yl)-1H-pyrazole-3-carbonyl)piperazin-1-yl)-7-oxo-[1,2,4]triazolo[1,5-a]pyrimidin-4(7H)-yl)acetamide

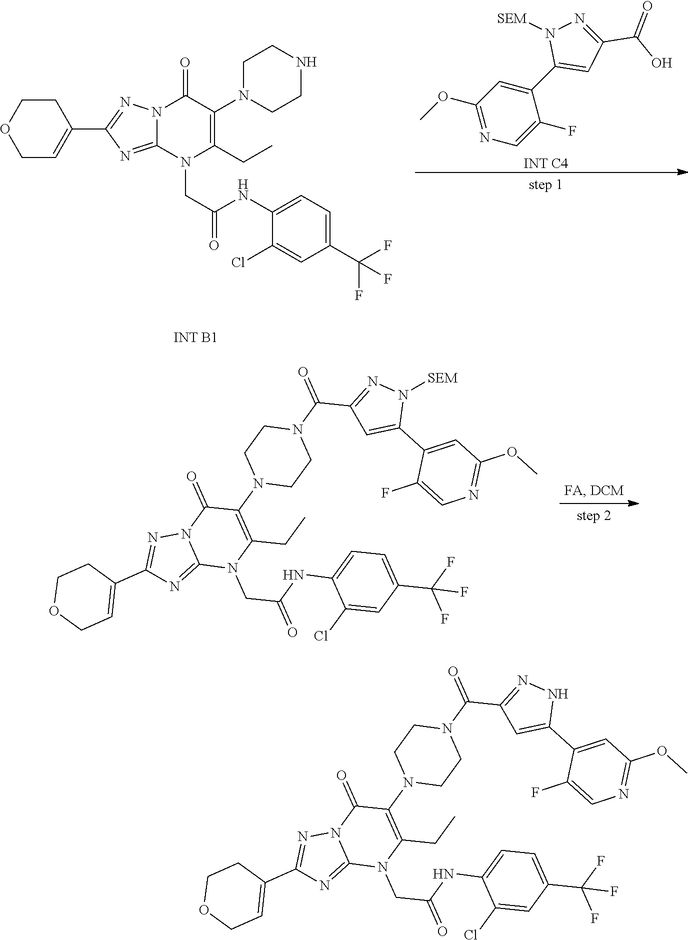

### Step 1: N-(2-chloro-4-(trifluoromethyl)phenyl)-2-(2-(3,6-dihydro-2H-pyran-4-yl)-5-ethyl-6-(4-(5-(5-fluoro-2-methoxypyridin-4-yl)-1-((2-(trimethylsilyl) ethoxy)methyl)-1H-pyrazole-3-carbonyl)piperazin-1-yl)-7-oxo-[1,2,4]triazolo[1,5-a]pyrimidin-4(7H)-yl)acetamide

This compound was prepared by procedures analogous to those described for method A using INT B1 and INT C4 to afford the title product as a white solid. LCMS calc. for C₄₁H₄₇ClF₄N₁₀O₆SiNa [M+Na]⁺: m/z=937.3; Found: 936.9.

### Step 2: N-(2-chloro-4-(trifluoromethyl)phenyl)-2-(2-(3,6-dihydro-2H-pyran-4-yl)-5-ethyl-6-(4-(5-(5-fluoro-2-methoxypyridin-4-yl)-1H-pyrazole-3-carbonyl)piperazin-1-yl)-7-oxo-[1,2,4]triazolo[1,5-a]pyrimidin-4(7H)-yl)acetamide

A mixture of N-(2-chloro-4-(trifluoromethyl)phenyl)-2-(2-(3,6-dihydro-2H-pyran-4-yl)-5-ethyl-6-(4-(5-(5-fluoro-2-methoxypyridin-4-yl)-1-((2-(trimethylsilyl) ethoxy)methyl)-1H-pyrazole-3-carbonyl)piperazin-1-yl)-7-oxo-[1,2,4]triazolo[1,5-a]pyrimidin-4(7H)-yl)acetamide (20 mg, 0.02 mmol) and FA (0.2 mL) in DCM (0.5 mL) was stirred at r.t. overnight. The mixture was concentrated under reduced pressure. The residue was purified by Prep-HPLC on a C18 column eluting with MeCN/H₂O (45-60%, with 0.1% FA) to afford the title compound (7.3 mg, 47% yield) as a white solid. ¹H NMR (400 MHZ, DMSO-d₆)₈ ppm 14.03 (s, 1H), 10.40 (s, 1H), 8.29 (s, 1H), 8.06 (d, J=8.6 Hz, 1H), 7.97 (s, 1H), 7.72 (d, J=8.7 Hz, 1H), 7.33 (d, J=5.1 Hz, 1H), 7.11 (s, 1H), 6.83 (s, 1H), 5.32 (s, 2H), 4.54 (s, 2H), 4.25 (s, 2H), 3.84 (d, J=30.1 Hz, 6H), 3.48 (d, J=10.8 Hz, 3H), 3.09-2.93 (m, 3H), 2.79 (s, 2H), 1.21 (t, J=7.6 Hz, 4H). LCMS calc. for C₃₅H₃₄ClF₄N₁₀O₅ [M+H]⁺: m/z=785.2; Found: 785.0.

### Example 10: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N,N-dimethylpiperazine-1-carboxamide

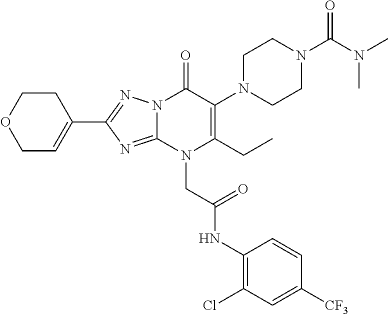 [SMILES: CCc1c(N2CCN(C(=O)N(C)C)CC2)c(=O)n2nc(C3=CCOCC3)nc2n1CC(=O)Nc1ccc(C(F)(F)F)cc1Cl]

To a mixture of INT B1 (15 mg, 0.027 mmol) and triethylamine (0.011 mL, 0.08 mmol) in DCM (2 mL) was added a solution of dimethylcarbamic chloride (3.4 mg, 0.03 mmol) in DCM (1 mL) at 0° C. The resulting mixture was stirred at r.t. for 2 h., quenched with H₂O (5 mL) and extracted with DCM (5 mL×3). The combined organic layers were washed with aq. NH₄Cl (10 mL) and brine (10 mL), dried over anhydrous Na₂SO₄, filtered and concentrated. The residue was purified by Prep-HPLC on a C18 column eluting with MeCN/H₂O (15-55%, with 0.1% FA) to afford the title compound (9.4 mg, 56% yield) as a white solid. ¹H NMR (400 MHZ, DMSO-d₆)₈ ppm 10.36 (s, 1H), 8.06 (d, J=8.9 Hz, 1H), 7.96 (s, 1H), 7.71 (d, J=9.7 Hz, 1H), 6.83 (s, 1H), 5.31 (s, 2H), 4.26 (s, 2H), 3.80 (t, J=5.5 Hz, 2H), 3.50 (dd, J=24.2, 12.9 Hz, 4H), 2.99-2.86 (m, 4H), 2.77 (s, 6H), 2.69-2.57 (m, 4H), 1.18 (t, J=7.5 Hz, 3H). LCMS calc. for C₂₈H₃₃ClF₃N₈O₄ [M+H]⁺: m/z=637.2; Found: 637.3.

### Example 11: N-(2-Chloro-4-(trifluoromethyl)phenyl)-2-(2-(3,6-dihydro-2H-pyran-4-yl)-5-ethyl-6-(4-(ethylsulfonyl)piperazin-1-yl)-7-oxo-[1,2,4]triazolo[1,5-a]pyrimidin-4(7H)-yl)acetamide

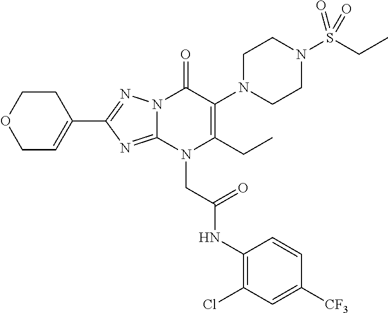 [SMILES: CCc1c(N2CCN(S(=O)(=O)CC)CC2)c(=O)n2nc(C3=CCOCC3)nc2n1CC(=O)Nc1ccc(C(F)(F)F)cc1Cl]

To a mixture of INT B1 (15 mg, 0.027 mmol) and triethylamine (0.011 mL, 0.08 mmol) in DCM (2 mL) were added a solution of ethanesulfonyl chloride (4.1 mg, 0.03 mmol) in DCM (1 mL) at 0° C. The resulting mixture was stirred at 0° C. for 2 h., quenched with H₂O (5 mL) and extracted with DCM (5 mL×3). The combined organic layers were washed with saturated aq. NH₄Cl (10 mL) and brine (10 mL), dried over anhydrous Na₂SO₄, filtered and concentrated. The residue was purified by Prep-HPLC on a C18 column eluting with MeCN/H₂O (15-55%, with 0.1% FA) to afford the title compound (9.7 mg, 56% yield) as a white solid. ¹H NMR (400 MHZ, DMSO-d₆) δ ppm 8.06 (d, J=8.3 Hz, 1H), 7.96 (s, 1H), 7.72 (d, J=10.0 Hz, 1H), 6.83 (s, 1H), 5.31 (s, 2H), 4.26 (d, J=2.1 Hz, 2H), 3.81 (t, J=5.1 Hz, 2H), 3.63-3.47 (m, 4H), 3.12 (q, J=7.2 Hz, 4H), 3.02-2.93 (m, 4H), 2.75 (d, J=11.9 Hz, 2H), 1.26 (d, J=7.3 Hz, 3H), 1.17 (t, J=7.5 Hz, 3H). LCMS calc. for C₂₇H₃₂ClF₃N₇O₅S [M+H]⁺: m/z=658.2; Found: 658.0.

### Example 12: N-(2-Chloro-4-(trifluoromethyl)phenyl)-2-(2-(3,6-dihydro-2H-pyran-4-yl)-5-ethyl-6-(4-(2-hydroxypropanoyl)piperazin-1-yl)-7-oxo-[1,2,4]triazolo[1,5-a]pyrimidin-4(7H)-yl)acetamide

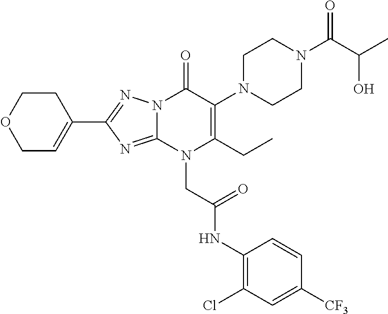 [SMILES: CCc1c(N2CCN(C(=O)C(C)O)CC2)c(=O)n2nc(C3=CCOCC3)nc2n1CC(=O)Nc1ccc(C(F)(F)F)cc1Cl]

To a solution of INT B1 (15 mg, 0.03 mmol) in DCM (4 mL) was added triethylamine (2.2 mg, 0.02 mmol). The mixture was stirred at r.t. for 0.5 h. To the above mixture was added 2-hydroxypropanoic acid (3.1 mg, 0.035 mmol), 1-hydroxybenzotriazole (4.4 mg, 0.03 mmol) and 1-ethyl-3-(3-dimethylaminopropyl) carbodiimide hydrochloride (8.1 mg, 0.04 mmol). The resulting mixture was stirred at r.t. for 2 h., washed with saturated NaHCO₃ aq. (5 mL), water (5 mL) and brine (5 mL). The organic layer was dried over anhydrous Na₂SO₄, filtered and concentrated under reduced pressure. The residue was purified by Prep-HPLC on a C18 column eluting with MeCN/H₂O (15-55%, with 0.1% FA) to afford the title compound (9.5 mg, 56% yield) as a white solid. ¹H NMR (400 MHz, DMSO-d₆) δ ppm 10.37 (s, 1H), 8.06 (d, J=8.7 Hz, 1H), 7.96 (s, 1H), 7.72 (d, J=8.3 Hz, 1H), 6.83 (s, 1H), 5.31 (s, 2H), 4.94 (dd, J=17.9, 7.1 Hz, 1H), 4.47 (s, 2H), 4.40 (s, 1H), 4.26 (s, 2H), 4.05 (s, 2H), 3.80 (t, J=5.4 Hz, 2H), 2.99 (d, J=7.1 Hz, 2H), 2.77 (s, 2H), 2.74-2.65 (m, 4H), 1.22 (d, J=12.0 Hz, 3H), 1.18 (d, J=7.5 Hz, 3H). LCMS calc. for C₂₈H₃₂ClF₃N₇₀₅ [M+H]⁺: m/z=638.2; Found: 638.2.

### Example 13: N-(2-Chloro-4-(trifluoromethyl)phenyl)-2-(6-(4-(cyclopropanecarbonyl)piperazin-1-yl)-2-(3,6-dihydro-2H-pyran-4-yl)-5-ethyl-7-oxo-[1,2,4]triazolo[1,5-a]pyrimidin-4(7H)-yl)acetamide

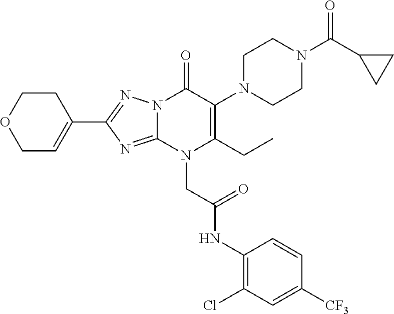 [SMILES: CCc1c(N2CCN(C(=O)C3CC3)CC2)c(=O)n2nc(C3=CCOCC3)nc2n1CC(=O)Nc1ccc(C(F)(F)F)cc1Cl]

To a solution of INT B1 (15.0 mg, 0.03 mmol), cyclopropanecarboxylic acid (3.4 mg, 0.04 mmol) and catalytic amount of DMAP (0.3 mg, 0.003 mmol) in DCM (4 mL) was added 1-ethyl-3-(3-dimethylaminopropyl) carbodiimide hydrochloride (7.5 mg, 0.04 mmol). The mixture was stirred at r.t. for 2 h. and concentrated. The residue was purified by Prep-HPLC on a C18 column eluting with MeCN/H₂O (15-55%, with 0.1% FA) to afford the title compound (9.3 mg, 55%) as a white solid. ¹H NMR (400 MHZ, DMSO-d₆)₈ ppm 10.36 (s, 1H), 8.06 (d, J=8.5 Hz, 1H), 7.97 (s, 1H), 7.72 (d, J=8.6 Hz, 1H), 6.83 (s, 1H), 5.32 (s, 2H), 4.30 (t, J=26.8 Hz, 5H), 3.80 (t, J=5.4 Hz, 2H), 3.42 (d, J=48.9 Hz, 2H), 2.99 (d, J=7.0 Hz, 2H), 2.70 (d, J=22.4 Hz, 4H), 2.03 (dd, J=12.6, 6.4 Hz, 1H), 1.21 (dd, J=15.3, 7.9 Hz, 4H), 0.74 (s, 4H). LCMS calc. for C₂₉H₃₂ClF₃N₇O₄ [M+H]⁺: m/z=634.2; Found: 634.3.

### Example 14: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-((1-methyl-1H-pyrazol-4-yl) sulfonyl)piperazine-1-carboxamide

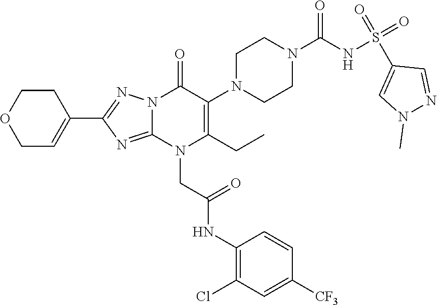 [SMILES: CCc1c(N2CCN(C(=O)NS(=O)(=O)c3cnn(C)c3)CC2)c(=O)n2nc(C3=CCOCC3)nc2n1CC(=O)Nc1ccc(C(F)(F)F)cc1Cl]

To a stirring solution of 1-methyl-1H-pyrazole-4-sulfonamide (100 mg, 0.62 mmol, INT C5) in DCM (4 mL) was added triphosgene (96 mg, 0.32 mmol) and TEA (250 mg, 2.48 mmol) at 0° C. under N₂ atmosphere. The resulting mixture was stirred for 0.5 h. at 0° C. Then INT B1 (20 mg, 0.02 mmol) in DCM (1 mL) was added at 0° C. The resulting mixture was stirred for 3 h. at 0° C. and concentrated under reduced pressure. The residue was purified by Prep-HPLC on a C18 column eluting with MeCN/H₂O (45-60%, with 0.1% NH₄HCO₃) to afford the title compound (10 mg, 2% yield) as a white solid. ¹H NMR (400 MHZ, DMSO-d₆)₈ ppm 10.35 (s, 1H), 8.06 (d, J=8.6 Hz, 1H), 7.97 (s, 1H), 7.72 (d, J=7.0 Hz, 1H), 7.61 (s, 1H), 7.08 (s, 1H), 6.82 (s, 1H), 5.30 (s, 2H), 4.25 (d, J=2.2 Hz, 2H), 4.10 (s, 2H), 3.81 (dd, J=13.1, 7.5 Hz, 6H), 2.96 (d, J=7.7 Hz, 3H), 2.76 (s, 2H), 2.56 (s, 5H), 1.18 (d, J=7.4 Hz, 3H). LCMS calc. for C₃₀H₃₃ClF₃N₁₀O₆S [M+H]⁺: m/z=753.2; Found: 753.4. Example 15:4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-ethylpiperazine-1-carboxamide

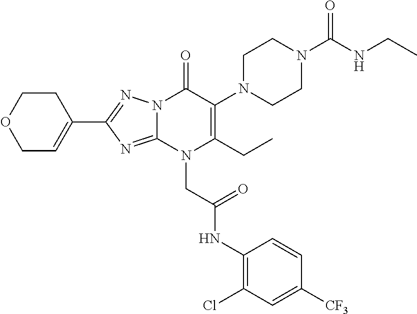 [SMILES: CCNC(=O)N1CCN(c2c(CC)n(CC(=O)Nc3ccc(C(F)(F)F)cc3Cl)c3nc(C4=CCOCC4)nn3c2=O)CC1]

To a stirred solution of isocyanatoethane (4.00 mg, 0.05 mmol) in DCM (1 mL) was added TEA (7.00 mg, 0.07 mmol) and INT B1 (10 mg, 0.02 mmol) in DCM (1 mL) at 0° C. under N₂ atmosphere.

The resulting mixture was stirred for additional 1 h. at 0° C. and then concentrated. The residue was purified by Prep-HPLC on a C18 column eluting with MeCN/H₂O (30-50%, with 0.1% NH₄HCO₃) to afford the title compound (12 mg, 70% yield) as a white solid. ¹H NMR (400 MHZ, DMSO-d₆) ¿ ppm 10.35 (s, 1H), 8.06 (d, J=8.5 Hz, 1H), 7.97 (s, 1H), 7.72 (d, J=8.6 Hz, 1H), 6.83 (s, 1H), 6.50 (t, J=5.4 Hz, 1H), 5.31 (s, 2H), 4.25 (d, J=2.5 Hz, 2H), 3.95 (d, J=12.0 Hz, 2H), 3.80 (t, J=5.4 Hz, 2H), 3.47-3.34 (m, 4H), 3.07 (dd, J=7.0, 5.6 Hz, 2H), 2.97 (d, J=7.4 Hz, 2H), 2.82 (t, J=11.3 Hz, 2H), 2.62 (d, J=11.1 Hz, 2H), 1.18 (t, J=7.5 Hz, 3H), 1.03 (t, J=7.1 Hz, 3H). LCMS calc. for C₂₈H₃₃ClF₃N₈O₄ [M+H]⁺: m/z=637.2; Found: 637.1.

### Example 16: N-(2-Chloro-4-(trifluoromethyl)phenyl)-2-(2-(3,6-dihydro-2H-pyran-4-yl)-5-ethyl-6-(4-(3-hydroxypropanoyl)piperazin-1-yl)-7-oxo-[1,2,4]triazolo[1,5-a]pyrimidin-4(7H)-yl)acetamide

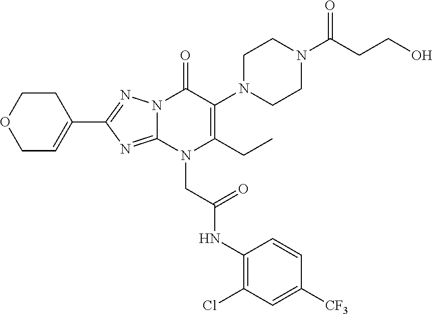 [SMILES: CCc1c(N2CCN(C(=O)CCO)CC2)c(=O)n2nc(C3=CCOCC3)nc2n1CC(=O)Nc1ccc(C(F)(F)F)cc1Cl]

To a stirred solution of INT B1 (15 mg, 0.03 mmol) and 3-hydroxypropanoic acid (3 mg, 0.03 mmol) in THF (3 mL) was added HATU (15 mg, 0.04 mmol) and TEA (9.8 mg, 0.08 mmol) at r.t.

The resulting mixture was stirred at r.t. for 12 h., and concentrated under reduced pressure. The residue was purified by Prep-HPLC on a C18 column eluting with MeCN/H₂O (30-50%, with 0.1% NH₄HCO₃) to afford the title compound (10.5 mg, 61% yield) as a white solid. ¹H NMR (400 MHZ, DMSO-d₆)₈ ppm 10.35 (s, 1H), 8.06 (d, J=8.6 Hz, 1H), 7.97 (d, J=1.5 Hz, 1H), 7.76-7.69 (m, 1H), 6.83 (s, 1H), 5.32 (s, 2H), 4.52 (t, J=5.4 Hz, 1H), 4.43 (d, J=11.7 Hz, 1H), 4.25 (d, J=2.4 Hz, 2H), 3.93 (d, J=12.8 Hz, 1H), 3.80 (t, J=5.4 Hz, 2H), 3.66 (dd, J=12.1, 6.5 Hz, 2H), 3.45 (t, J=10.6 Hz, 3H), 3.19 (t, J=11.5 Hz, 1H), 3.05-2.92 (m, 2H), 2.71 (t, J=10.8 Hz, 3H), 2.55 (d, J=6.9 Hz, 2H), 1.19 (t, J=7.5 Hz, 3H). LCMS calc. for C₂₈H₃₂ClF₃N₇₀₅ [M+H]⁺: m/z=638.2; Found: 638.3.

### Example 17: 2-(6-(4-(1H-Pyrazolo[4,3-d]pyrimidine-7-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

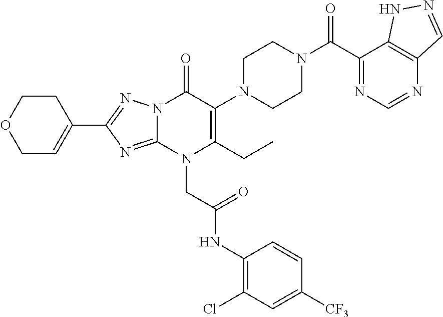 [SMILES: CCc1c(N2CCN(C(=O)c3ncnc4cn[nH]c34)CC2)c(=O)n2nc(C3=CCOCC3)nc2n1CC(=O)Nc1ccc(C(F)(F)F)cc1Cl]

### Step 1: N-(2-chloro-4-(trifluoromethyl)phenyl)-2-(2-(3,6-dihydro-2H-pyran-4-yl)-5-ethyl-7-oxo-6-(4-(1-((2-(trimethylsilyl) ethoxy)methyl)-1H-pyrazolo[4,3-d]pyrimidine-7-carbonyl)piperazin-1-yl)-[1,2,4]triazolo[1,5-a]pyrimidin-4(7H)-yl)acetamide

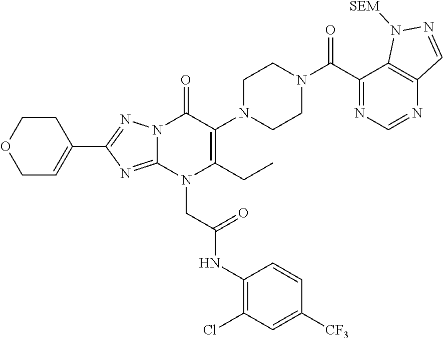 [SMILES: CCc1c(N2CCN(C(=O)c3ncnc4cnn(COCC[Si](C)(C)C)c34)CC2)c(=O)n2nc(C3=CCOCC3)nc2n1CC(=O)Nc1ccc(C(F)(F)F)cc1Cl]

This compound was prepared by procedures analogous to those described for method A using 1-((2-(trimethylsilyl) ethoxy)methyl)-1H-pyrazolo[4,3-d]pyrimidine-7-carboxylic acid and INT B1 to give the title product as a yellow oil. LCMS calc. for C₃₇H₄₂ClF₃N₁₁O₅Si [M−H]⁻: m/z=840.3; Found: 840.0.

### Step 2: 2-(6-(4-(1H-pyrazolo[4,3-d]pyrimidine-7-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

A solution of N-(2-chloro-4-(trifluoromethyl)phenyl)-2-(2-(3,6-dihydro-2H-pyran-4-yl)-5-ethyl-7-oxo-6-(4-(1-((2-(trimethylsilyl) ethoxy)methyl)-1H-pyrazolo[4,3-d]pyrimidine-7-carbonyl)piperazin-1-yl)-[1,2,4]triazolo[1,5-a]pyrimidin-4(7H)-yl)acetamide (40 mg, 0.05 mmol) in 4 M HCl/dioxane (2 mL) was stirred at r.t. overnight. The mixture was concentrated under reduced pressure. The residue was purified by Prep-HPLC on a C18 column eluting with MeCN/H₂O (15-60%, with 0.1% FA) to give the title compound (18.8 mg, 55%) as a white solid. ¹H NMR (400 MHZ, DMSO-d₆) δ ppm 14.16 (s, 1H), 10.43 (s, 1H), 9.10 (s, 1H), 8.55 (s, 1H), 8.05 (d, J=8.4 Hz, 1H), 7.96 (s, 1H), 7.71 (d, J=8.4 Hz, 1H), 6.83 (s, 1H), 5.32 (s, 2H), 4.65 (d, J=12.0 Hz, 1H), 4.25 (s, 2H), 4.06 (d, J=11.8 Hz, 1H), 3.80 (s, 2H), 3.54 (dd, J=26.4, 12.3 Hz, 4H), 3.19-2.86 (m, 5H), 2.68 (d, J=10.4 Hz, 1H), 1.20 (t, J=7.2 Hz, 3H). LCMS calc. for C₃₁H₃₀ClF₃N₁₁₀₄ [M+H]⁺: m/z=712.2; Found: 712.2.

### Example 18: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(4-(trifluoromethyl)-1H-pyrazol-5-yl)piperazine-1-carboxamide and Example 19: N-(2-Chloro-4-(trifluoromethyl)phenyl)-2-(2-(3,6-dihydro-2H-pyran-4-yl)-5-ethyl-7-oxo-6-(4-(4-(trifluoromethyl)-1H-pyrazole-5-carbonyl)piperazin-1-yl)-[1,2,4]triazolo[1,5-a]pyrimidin-4(7H)-yl)acetamide

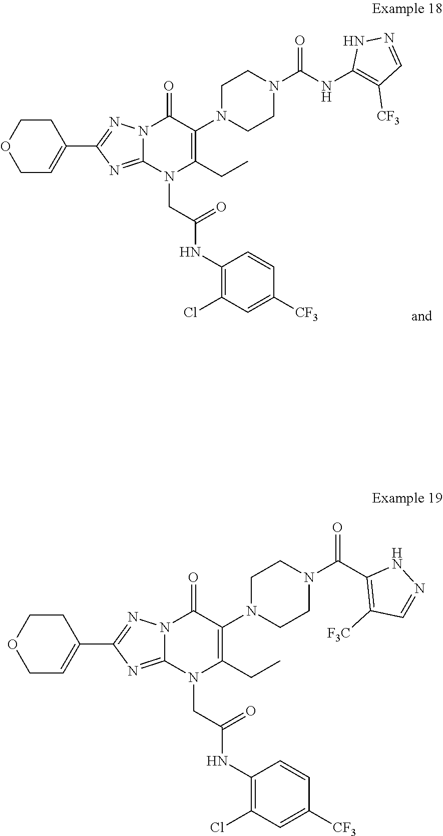 [SMILES: CCc1c(N2CCN(C(=O)Nc3[nH]ncc3C(F)(F)F)CC2)c(=O)n2nc(C3=CCOCC3)nc2n1CC(=O)Nc1ccc(C(F)(F)F)cc1Cl ; "and" ; CCc1c(N2CCN(C(=O)c3[nH]ncc3C(F)(F)F)CC2)c(=O)n2nc(C3=CCOCC3)nc2n1CC(=O)Nc1ccc(C(F)(F)F)cc1Cl]

### Step 1:4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(1-(4-methoxybenzyl)-4-(trifluoromethyl)-1H-pyrazol-5-yl)piperazine-1-carboxamide (Compound A) and N-(2-chloro-4-(trifluoromethyl)phenyl)-2-(2-(3,6-dihydro-2H-pyran-4-yl)-5-ethyl-6-(4-(1-(4-methoxybenzyl)-4-(trifluoromethyl)-1H-pyrazole-5-carbonyl)piperazin-1-yl)-7-oxo-[1,2,4]triazolo[1,5-a]pyrimidin-4(7H)-yl)acetamide (Compound B)

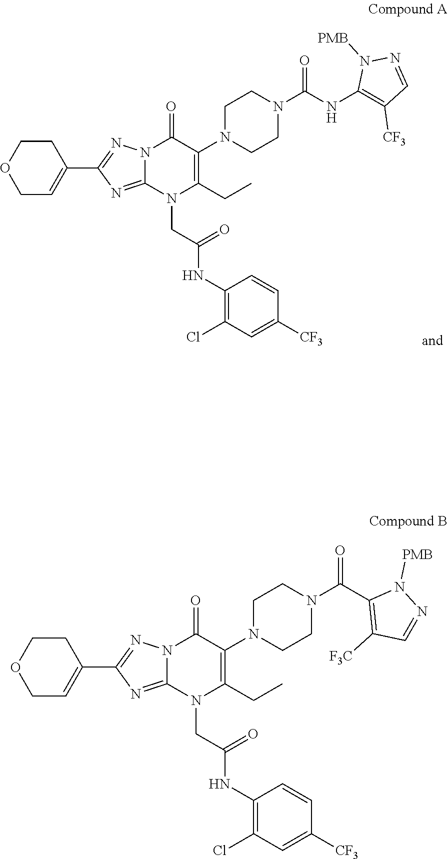

To a solution of 1-(4-methoxybenzyl)-4-(trifluoromethyl)-1H-pyrazole-5-carboxylic acid (50 mg, 0.17 mmol) was added TEA (24 mg, 0.24 mmol) and DPPA (66 mg, 0.24 mmol). The mixture was stirred at r.t. for 30 min., and then INT B1 (45 mg, 0.08 mmol) was added. The mixture was stirred at 90° C. for 3 h. and diluted with water (30 mL) and extracted with EtOAc (10 mL×3). The combined organic layers were dried over anhydrous Na₂SO₄ and concentrated under reduced pressure.

The residue was purified by flash chromatography on a silica gel column eluting with MeOH/DCM (5%) to afford the compound A (the earlier eluted fraction, 10 mg, 6.8% yield) as a white solid and compound B (the latter eluted fraction, 20 mg, 13.7% yield) as a white solid. Compound A: LCMS calc. for C₃₈H₃₈ClF₆N₁₀O₅ [M+H]⁺: m/z=863.3; Found: 862.7. Compound B: LCMS calc. for C₃₈H₃₇ClF₆N₉O₅ [M+H]⁺: m/z=848.2; Found: 847.7.

### Step 2: 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(4-(trifluoromethyl)-1H-pyrazol-5-yl)piperazine-1-carboxamide (Example 18)

A mixture of compound A (10 mg, 0.012 mmol) in TFA (1 mL) was stirred at 70° C. for 1 h. and evaporated under reduced pressure. The residue was purified by Prep-HPLC on a C18 column eluting with MeCN/H₂O (30%-50%) to afford the title compound (2 mg, 27% yield) as a white solid. ¹H NMR (400 MHZ, CD₃OD) δ ppm 8.17 (d, J=8.6 Hz, 1H), 8.03 (s, 1H), 7.82 (d, J=1.5 Hz, 1H), 7.65-7.59 (m, 1H), 6.95 (s, 1H), 5.39 (s, 2H), 4.32 (d, J=2.7 Hz, 2H), 4.15 (d, J=12.7 Hz, 2H), 3.89 (t, J=5.4 Hz, 2H), 3.74-3.64 (m, 2H), 3.22-3.06 (m, 4H), 2.82 (d, J=11.3 Hz, 2H), 2.63 (s, 2H), 1.32 (t, J=7.5 Hz, 3H). LCMS calc. for C₃₀H₃₀ClF₆N₁₀O₄ [M+H]⁺: m/z=743.2; Found: 742.8.

### Step 3: N-(2-chloro-4-(trifluoromethyl)phenyl)-2-(2-(3,6-dihydro-2H-pyran-4-yl)-5-ethyl-7-oxo-6-(4-(4-(trifluoromethyl)-1H-pyrazole-5-carbonyl)piperazin-1-yl)-[1,2,4]triazolo[1,5-a]pyrimidin-4 (7H)-yl)acetamide (Example 19)

This compound was prepared as a white solid by procedures analogous to those described for Example 18 using compound B to replace compound A. ¹H NMR (400 MHZ, DMSO-d₆) δ ppm 8.45 (s, 1H), 8.05 (d, J=8.5 Hz, 1H), 7.95 (s, 1H), 7.76-7.66 (m, 1H), 6.83 (s, 1H), 5.33 (s, 2H), 4.53 (d, J=12.0 Hz, 1H), 4.32-4.18 (m, 2H), 3.80 (t, J=5.3 Hz, 2H), 3.69 (d, J=11.9 Hz, 1H), 3.43 (d, J=11.5 Hz, 3H), 3.29-3.18 (m, 2H), 2.98 (t, J=9.6 Hz, 3H), 2.73 (dd, J=53.2, 10.6 Hz, 2H), 1.18 (t, J=7.4 Hz, 3H). LCMS calc. for C₃₀H₂₉ClF₆N₉O₄ [M+H]⁺: m/z=728.2; Found: 727.8.

### Example 20: 2-(6-(4-(7H-Purine-6-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

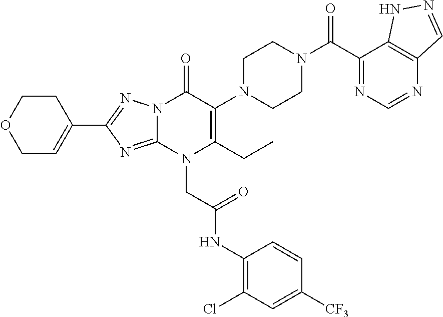 [SMILES: CCc1c(N2CCN(C(=O)c3ncnc4cn[nH]c34)CC2)c(=O)n2nc(C3=CCOCC3)nc2n1CC(=O)Nc1ccc(C(F)(F)F)cc1Cl]

### Step 1: N-(2-chloro-4-(trifluoromethyl)phenyl)-2-(2-(3,6-dihydro-2H-pyran-4-yl)-5-ethyl-7-oxo-6-(4-(7-((2-(trimethylsilyl) ethoxy)methyl)-7H-purine-6-carbonyl)piperazin-1-yl)-[1,2,4]triazolo[1,5-a]pyrimidin-4(7H)-yl)acetamide

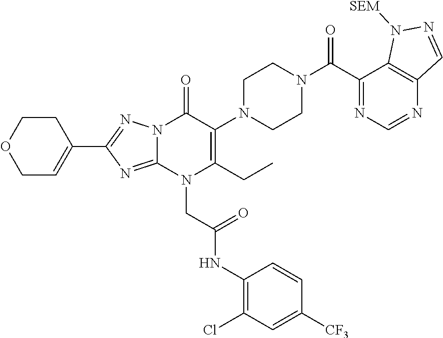 [SMILES: CCc1c(N2CCN(C(=O)c3ncnc4cnn(COCC[Si](C)(C)C)c34)CC2)c(=O)n2nc(C3=CCOCC3)nc2n1CC(=O)Nc1ccc(C(F)(F)F)cc1Cl]

To a solution of 7-((2-(trimethylsilyl) ethoxy)methyl)-7H-purine-6-carboxylic acid (30 mg, 0.06 mmol, INT C21), N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide (48 mg, 0.03 mmol) and HOBt (24 mg, 0.06 mmol) in DMF (1 mL) was added EDCI (15 mg, 0.06 mmol) at 0° C. The resulting mixture was stirred for 2 h. at r.t., and diluted with H₂O (2 mL). The mixture was filtered to afford the title compound (25 mg, 49% yield) as a yellow solid. LCMS calc. for C₃₇H₄₄ClF₃N₁₁O₅Si [M+H]⁺: m/z=842.2; Found: 842.2.

### Step 2: 2-(6-(4-(7H-purine-6-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoro methyl)phenyl)acetamide

A solution of N-(2-chloro-4-(trifluoromethyl)phenyl)-2-(2-(3,6-dihydro-2H-pyran-4-yl)-5-ethyl-7-oxo-6-(4-(7-((2-(trimethylsilyl) ethoxy)methyl)-7H-purine-6-carbonyl)piperazin-1-yl)-[1,2,4]triazolo[1,5-a]pyrimidin-4(7H)-yl)acetamide (20 mg, 0.02 mmol) in 4M HCl/dioxane (1 mL) was stirred at r.t. for 2 h., and concentrated under reduced pressure. The residue was purified by Prep-HPLC on a C18 column eluting with MeCN/H₂O (30-50%, with 0.1% NH₄HCO₃) to yield the title compound (17 mg, 81% yield) as a white solid. ¹H NMR (400 MHZ, DMSO-d₆) δ ppm 10.37 (s, 1H), 8.98 (s, 1H), 8.71 (s, 1H), 8.05 (d, J=8.4 Hz, 1H), 7.96 (s, 1H), 7.71 (d, J=8.8 Hz, 1H), 6.83 (s, 1H), 5.32 (s, 2H), 4.62 (d, J=12.0 Hz, 1H), 4.25 (d, J=2.4 Hz, 2H), 3.82 (s, 1H), 3.80 (d, J=5.4 Hz, 2H), 3.28 (d, J=10.0 Hz, 2H), 3.14-3.06 (m, 2H), 3.00 (d, J=8.1 Hz, 2H), 2.87 (d, J=11.7 Hz, 1H), 2.62 (d, J=11.0 Hz, 2H), 1.22 (d, J=11.1 Hz, 2H), 1.18 (d, J=7.6 Hz, 3H). LCMS calc. for C₃₁H₃₀ClF₃N₁₁₀₄ [M+H]⁺: m/z=712.2; Found: 712.3.

### Example 21: (Z)—N-(2-Chloro-4-(trifluoromethyl)phenyl)-2-(2-(3,6-dihydro-2H-pyran-4-yl)-5-ethyl-6-(4-((3-fluoropyridin-2-yl) (hydroxyimino) methyl)piperazin-1-yl)-7-oxo-[1,2,4]triazolo[1,5-a]pyrimidin-4(7H)-yl)acetamide

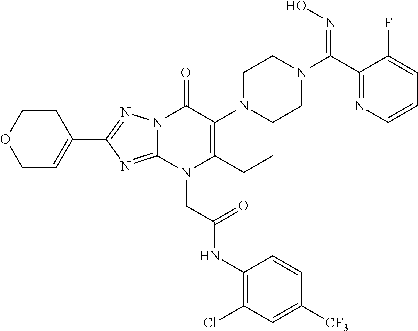 [SMILES: CCc1c(N2CCN(/C(=N\O)c3ncccc3F)CC2)c(=O)n2nc(C3=CCOCC3)nc2n1CC(=O)Nc1ccc(C(F)(F)F)cc1Cl]

To a solution of INT B1 (15 mg, 0.03 mmol) in DCM (2 mL) were added INT C11 (40 mg, 0.23 mmol) and TEA (15 mg, 0.15 mmol). The mixture was stirred at r.t. overnight, and concentrated under reduced pressure. The residue was purified by Prep-HPLC on a C18 column eluting with MeCN/H₂O (15%-55%, with 0.1% FA) to afford the title compound (6 mg, 32% yield) as a white solid. ¹H NMR (400 MHZ, DMSO-d₆) δ ppm 10.35 (s, 1H), 9.50 (s, 1H), 8.52 (dt, J=4.8, 1.6 Hz, 1H), 8.05 (d, J=8.6 Hz, 1H), 7.96 (d, J=2.0 Hz, 1H), 7.84-7.75 (m, 1H), 7.71 (dd, J=8.9, 2.1 Hz, 1H), 7.54 (dt, J=8.8, 4.5 Hz, 1H), 6.85-6.79 (m, 1H), 5.30 (s, 2H), 4.25 (d, J=2.9 Hz, 2H), 3.80 (t, J=5.5 Hz, 2H), 3.55 (dd, J=13.2, 10.1 Hz, 3H), 3.28 (s, 4H), 3.01-2.91 (m, 2H), 2.84 (dd, J=13.4, 10.5 Hz, 2H), 2.63 (d, J=11.0 Hz, 2H), 1.17 (t, J=7.5 Hz, 3H). LCMS calc. for C₃₁H₃₁ClF₄N₉O₄ [M+H]⁺: m/z=704.2; Found: 703.8.

### Example 22: N-(2-Chloro-4-(trifluoromethyl)phenyl)-2-(2-(3,6-dihydro-2H-pyran-4-yl)-6-(4-(3,5-dioxo-2,3,4,5-tetrahydro-1,2,4-triazine-6-carbonyl)piperazin-1-yl)-5-ethyl-7-oxo-[1,2,4]triazolo[1,5-a]pyrimidin-4(7H)-yl)acetamide

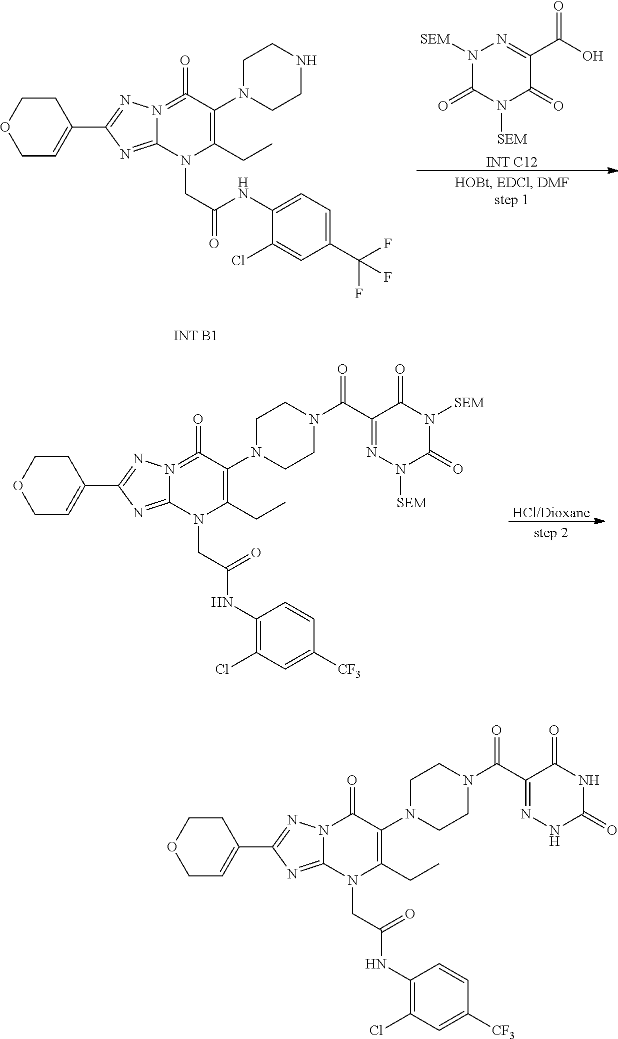

### Step 1: N-(2-chloro-4-(trifluoromethyl)phenyl)-2-(2-(3,6-dihydro-2H-pyran-4-yl)-6-(4-(3,5-dioxo-2,4-bis((2-(trimethylsilyl) ethoxy)methyl)-2,3,4,5-tetrahydro-1,2,4-triazine-6-carbonyl)piperazin-1-yl)-5-ethyl-7-oxo-[1,2,4]triazolo[1,5-a]pyrimidin-4(7H)-yl)acetamide

This compound was prepared by procedures analogous to those described for method A using INT B1 and INT C12 to afford the title compound as a white solid.

### Step 2: N-(2-chloro-4-(trifluoromethyl)phenyl)-2-(2-(3,6-dihydro-2H-pyran-4-yl)-6-(4-(3,5-dioxo-2,3,4,5-tetrahydro-1,2,4-triazine-6-carbonyl)piperazin-1-yl)-5-ethyl-7-oxo-[1,2,4]triazolo[1,5-a]pyrimidin-4(7H)-yl)acetamide

A mixture of N-(2-chloro-4-(trifluoromethyl)phenyl)-2-(2-(3,6-dihydro-2H-pyran-4-yl)-6-(4-(3,5-dioxo-2,4-bis((2-(trimethylsilyl) ethoxy)methyl)-2,3,4,5-tetrahydro-1,2,4-triazine-6-carbonyl)piperazin-1-yl)-5-ethyl-7-oxo-[1,2,4]triazolo[1,5-a]pyrimidin-4(7H)-yl)acetamide (20 mg, 0.02 mmol) in 2.5 M HCl/dioxane (1 mL) was stirred at r.t. overnight. The reaction mixture was concentrated. The residue was purified by Prep-HPLC on a C18 column eluting with MeCN/H₂O (15-52%, with 0.1% FA) to afford the title compound (5 mg, 34% yield) as a white solid. ¹H NMR (400 MHz, DMSO-d₆) δ ppm 12.42 (d, J=56.0 Hz, 1H), 10.37 (s, 1H), 8.06 (d, J=8.6 Hz, 1H), 7.96 (d, J=2.1 Hz, 1H), 7.72 (dd, J=8.7, 2.2 Hz, 1H), 6.86-6.81 (m, 1H), 6.81-6.49 (m, 1H), 5.32 (s, 2H), 5.25-5.07 (m, 2H), 4.42 (d, J=12.5 Hz, 1H), 4.25 (q, J=2.9 Hz, 2H), 3.90-3.74 (m, 3H), 3.48-3.39 (m, 2H), 3.26-3.18 (m, 1H), 3.05-2.91 (m, 3H), 2.79 (d, J=11.4 Hz, 1H), 2.70-2.61 (m, 1H), 1.19 (t, J=7.6 Hz, 3H). LCMS calc. for C₂₉H₂₉ClF₃N₁₀O₀₆ [M+H]⁺: m/z=705.2; Found: 704.9.

### Example 23: N-(2-Chloro-4-(trifluoromethyl)phenyl)-2-(2-(3,6-dihydro-2H-pyran-4-yl)-5-ethyl-6-(4-(isoxazolo[4,5-b]pyridin-3-yl)piperazin-1-yl)-7-oxo-[1,2,4]triazolo[1,5-a]pyrimidin-4 (7H)-yl)acetamide

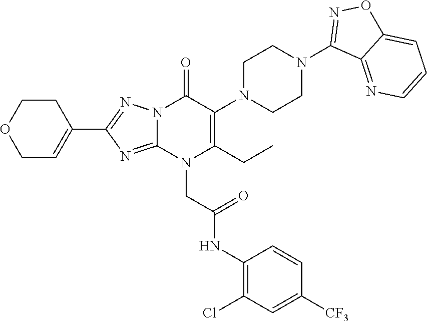 [SMILES: CCc1c(N2CCN(c3noc4cccnc34)CC2)c(=O)n2nc(C3=CCOCC3)nc2n1CC(=O)Nc1ccc(C(F)(F)F)cc1Cl]

To a solution of (Z)—N-(2-chloro-4-(trifluoromethyl)phenyl)-2-(2-(3,6-dihydro-2H-pyran-4-yl)-5-ethyl-6-(4-((3-fluoropyridin-2-yl) (hydroxyimino) methyl)piperazin-1-yl)-7-oxo-[1,2,4]triazolo[1,5-a]pyrimidin-4(7H)-yl)acetamide (Example 21) (20 mg, 0.03 mmol) in DMF (2 mL) was added t-BuOK (10 mg, 0.09 mmol). The mixture was stirred at 50° C. overnight and concentrated. The resulting residue was purified by Prep-HPLC on a C18 column eluting with MeCN/H₂O (20-55%, with 0.1% FA) to afford the title compound (5 mg, 26% yield) as a white solid. ¹H NMR (400 MHZ, DMSO-d₆) δ ppm 10.37 (s, 1H), 8.64 (dd, J=4.5, 1.2 Hz, 1H), 8.11 (dd, J=8.6, 1.3 Hz, 1H), 8.07 (d, J=8.6 Hz, 1H), 7.96 (d, J=2.1 Hz, 1H), 7.72 (dd, J=8.8, 2.1 Hz, 1H), 7.64 (dd, J=8.6, 4.4 Hz, 1H), 6.86-6.81 (m, 1H), 5.33 (s, 2H), 4.60 (d, J=12.0 Hz, 2H), 4.25 (q, J=2.8 Hz, 2H), 3.80 (t, J=5.5 Hz, 2H), 3.77-3.67 (m, 2H), 3.29-3.20 (m, 2H), 3.03 (q, J=7.4 Hz, 2H), 2.82 (d, J=11.2 Hz, 2H), 1.22 (t, J=7.4 Hz, 3H). LCMS calc. for C₃₁H₃₀ClF₃N₉O₄ [M+H]⁺: m/z=684.2; Found: 684.2.

### Example 24: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(cyclopropylmethyl)piperazine-1-carboxamide

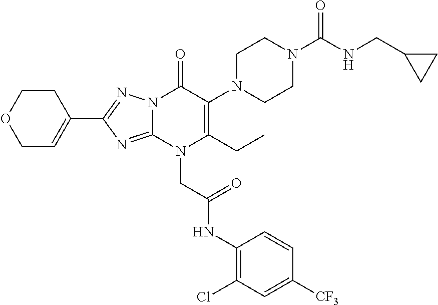 [SMILES: CCc1c(N2CCN(C(=O)NCC3CC3)CC2)c(=O)n2nc(C3=CCOCC3)nc2n1CC(=O)Nc1ccc(C(F)(F)F)cc1Cl]

To a stirred solution of cyclopropylmethanamine (150 mg, 2.11 mmol) and triphosgene (312 mg, 1.05 mmol) in DCM (3 mL) was added TEA (855 mg, 8.47 mmol) at 0° C. under N₂ atmosphere. The resulting mixture was stirred at 0° C. for 0.5 h., and then INT B1 (15 mg, 0.03 mmol) in DCM (1 mL) was added. The mixture was stirred for additional 2 h. at 0° C. and concentrated under reduced pressure. The residue was purified by Prep-HPLC on a C18 column eluting with MeCN/H₂O (30-50%, with 0.1% NH₄HCO₃) to afford the title compound (5.4 mg, 30% yield) as a white solid. ¹H NMR (400 MHz, DMSO-d₆) δ ppm 10.36 (s, 1H), 8.06 (d, J=8.5 Hz, 1H), 7.97 (d, J=1.5 Hz, 1H), 7.77-7.69 (m, 1H), 6.83 (s, 1H), 6.59 (t, J=5.6 Hz, 1H), 5.31 (s, 2H), 4.25 (d, J=2.5 Hz, 2H), 3.97 (d, J=12.2 Hz, 2H), 3.80 (t, J=5.4 Hz, 2H), 3.41 (t, J=10.4 Hz, 2H), 2.97 (ddd, J=18.5, 11.3, 6.5 Hz, 5H), 2.83 (t, J=11.1 Hz, 2H), 2.68-2.60 (m, 2H), 1.18 (t, J=7.5 Hz, 3H), 0.95 (d, J=6.8 Hz, 2H), 0.38 (dd, J=8.1, 1.6 Hz, 2H), 0.16 (dd, J=4.8, 1.3 Hz, 2H). LCMS calc. for C₃₀H₃₅ClF₃N₈O₄ [M+H]⁺: m/z=663.2; Found: 663.3.

### Example 25: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(ethylsulfonyl)piperazine-1-carboxamide

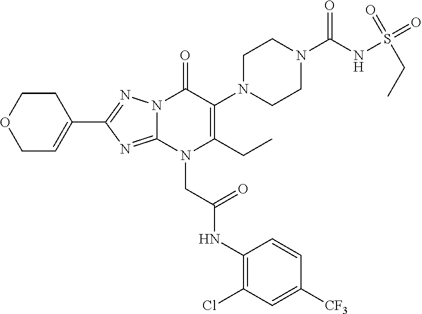 [SMILES: CCc1c(N2CCN(C(=O)NS(=O)(=O)CC)CC2)c(=O)n2nc(C3=CCOCC3)nc2n1CC(=O)Nc1ccc(C(F)(F)F)cc1Cl]

A mixture of INT B1 (50 mg, 0.09 mmol), ethanesulfonamide (14.4 mg, 0.13 mmol), triphosgene (15.67 mg, 0.05 mmol) in DCM (3 mL) was stirred at −78° C. for 10 min., and then TEA (140 mg, 0.40 mmol) was added. The mixture was stirred at r.t. for 2 h., quenched with H₂O (1 mL). The organic layer was separated and concentrated. The residue was purified by Prep-HPLC on a C18 column eluting with MeCN/H₂O (30-60% with 10 mM NH₄HCO₃) to afford the title compound (20 mg, 33%) as a white solid. ¹H NMR (400 MHZ, DMSO-d₆)₈ ppm 10.43 (d, J=60.2 Hz, 2H), 8.06 (d, J=8.6 Hz, 1H), 7.97 (d, J=2.1 Hz, 1H), 7.72 (dd, J=8.8, 2.1 Hz, 1H), 6.83 (s, 1H), 5.31 (s, 2H), 4.25 (d, J=2.9 Hz, 2H), 4.04 (d, J=12.6 Hz, 2H), 3.81 (d, J=5.8 Hz, 2H), 3.41 (d, J=11.2 Hz, 4H), 3.01-2.88 (m, 4H), 2.67 (d, J=10.5 Hz, 4H), 2.00 (q, J=7.0, 6.4 Hz, 1H), 1.19 (dd, J=17.3, 7.7 Hz, 6H). LCMS calc. for C₂₈H₃₃ClF₃N₈O₆S [M+H]⁺: m/z=701.2; Found: 700.8.

### Example 26: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(1-(4-methoxybenzyl)-4-(trifluoromethyl)-1H-imidazol-5-yl)piperazine-1-carboxamide

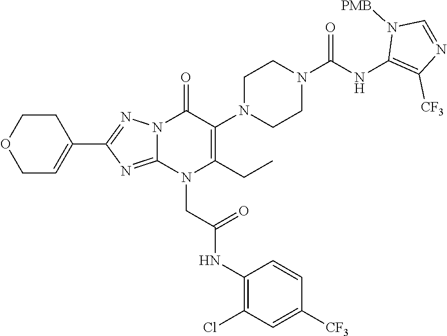

To a solution of INT C14 (50 mg, 0.17 mmol) in 1, 4-dioxane (2 mL) was added TEA (24 mg, 0.24 mmol) and DPPA (66 mg, 0.24 mmol). The mixture was stirred at r.t. for 30 min. Then INT B1 (45 mg, 0.08 mmol) was added and the mixture was stirred at 90° C. for 3 h., diluted with water (30 mL) and extracted with EtOAc (10 mL×3). The combined organic layers were dried over anhydrous Na₂SO₄, filtered and evaporated under reduced pressure. The residue was purified by flash chromatography on a silica gel column eluting with MeOH/DCM (5%) to afford the title compound (30 mg, 43% yield) as a white solid. ¹H NMR (400 MHZ, DMSO-d₆) δ ppm 10.37 (s, 1H), 8.51 (s, 1H), 8.06 (d, J=8.5 Hz, 1H), 7.97 (s, 1H), 7.84 (s, 1H), 7.73 (d, J=7.0 Hz, 1H), 7.27 (d, J=8.7 Hz, 2H), 6.95 (d, J=8.7 Hz, 2H), 6.84 (s, 1H), 5.33 (s, 2H), 4.98 (s, 2H), 4.29-4.22 (m, 2H), 4.07 (d, J=12.2 Hz, 2H), 3.81 (t, J=5.4 Hz, 2H), 3.74 (s, 3H), 3.50 (t, J=10.7 Hz, 2H), 3.29 (s, 2H), 3.01 (t, J=9.4 Hz, 4H), 2.71 (d, J=10.8 Hz, 2H), 1.20 (t, J=7.5 Hz, 3H). LCMS calc. for C₃₈H₃₈ClF₆N₁₀O₅ [M+H]⁺: m/z=863.3; Found: 863.8.

### Example 27: N-(2-Chloro-4-(trifluoromethyl)phenyl)-2-(2-(3,6-dihydro-2H-pyran-4-yl)-5-ethyl-7-oxo-6-(4-propionylpiperazin-1-yl)-[1,2,4]triazolo[1,5-a]pyrimidin-4(7H)-yl)acetamide

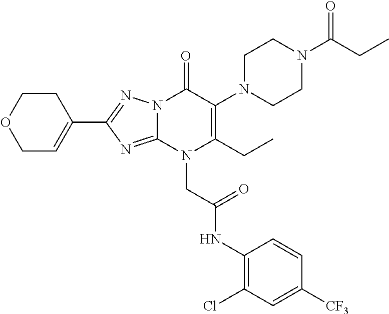 [SMILES: CCC(=O)N1CCN(c2c(CC)n(CC(=O)Nc3ccc(C(F)(F)F)cc3Cl)c3nc(C4=CCOCC4)nn3c2=O)CC1]

This compound was prepared by procedures analogous to those described for Example 13 using INT B1 and propionic acid to afford the title product as a white solid. ¹H NMR (400 MHZ, DMSO-d₆) δ ppm 10.37 (s, 1H), 8.06 (d, J=8.6 Hz, 1H), 7.96 (s, 1H), 7.72 (d, J=8.6 Hz, 1H), 6.83 (s, 1H), 5.32 (s, 2H), 4.42 (d, J=12.4 Hz, 1H), 4.25 (s, 2H), 3.88 (d, J=12.7 Hz, 1H), 3.80 (t, J=5.7 Hz, 2H), 3.52-3.37 (m, 4H), 3.20 (d, J=12.9 Hz, 2H), 2.98 (q, J=8.0, 7.5 Hz, 2H), 2.69 (d, J=11.5 Hz, 3H), 2.36 (dt, J=15.9, 7.9 Hz, 3H), 1.18 (t, J=7.6 Hz, 3H), 1.02 (t, J=7.3 Hz, 3H). LCMS calc. for C₂₈H₃₂ClF₃N₇O₄ [M+H]⁺: m/z=622.0; Found: 622.1.

### Example 28: N-(2-Chloro-4-(trifluoromethyl)phenyl)-2-(2-(3,6-dihydro-2H-pyran-4-yl)-5-ethyl-7-oxo-6-(4-sulfamoylpiperazin-1-yl)-[1,2,4]triazolo[1,5-a]pyrimidin-4(7H)-yl)acetamide

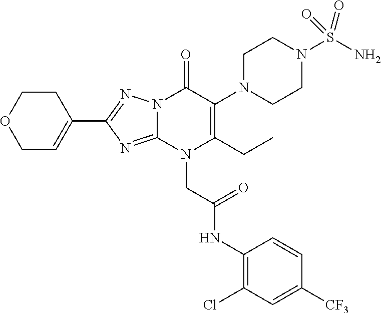 [SMILES: CCc1c(N2CCN(S(N)(=O)=O)CC2)c(=O)n2nc(C3=CCOCC3)nc2n1CC(=O)Nc1ccc(C(F)(F)F)cc1Cl]

To a solution of INT B1 (25 mg, 0.04 mmol) and 4-nitrophenyl sulfamate (14.4 mg, 0.07 mmol) in MeCN (3 mL) was added DIPEA (17.1 mg, 0.13 mmol). The mixture was stirred at r.t. overnight, and concentrated under reduced pressure. The residue was purified by Prep-HPLC on a C18 column eluting with MeCN/H₂O (30-50%, with 0.1% FA) to afford the title compound (5 mg, 31% yield) as a white solid. ¹H NMR (400 MHZ, DMSO-d₆) δ ppm 10.36 (s, 1H), 8.06 (d, J=8.6 Hz, 1H), 7.96 (s, 1H), 7.71 (d, J=8.5 Hz, 1H), 6.84 (s, 3H), 5.31 (s, 2H), 4.25 (s, 2H), 3.82 (d, J=13.3 Hz, 2H), 3.57 (t, J=10.7 Hz, 2H), 3.43 (d, J=10.1 Hz, 2H), 3.29 (s, 2H), 2.94 (d, J=7.4 Hz, 2H), 2.73 (dd, J=27.1, 11.7 Hz, 4H), 1.17 (t, J=7.2 Hz, 3H). LCMS calc. for C₂₅H₂₉ClF₃N₈O₅ [M+H]⁺: m/z=645.2; Found: 645.0.

### Example 29: N-((4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl) sulfonyl) propionamide

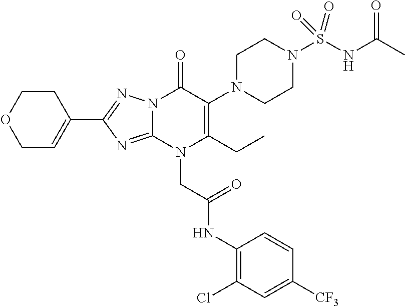 [SMILES: CCc1c(N2CCN(S(=O)(=O)NC(C)=O)CC2)c(=O)n2nc(C3=CCOCC3)nc2n1CC(=O)Nc1ccc(C(F)(F)F)cc1Cl]

### Step 1: Propionylsulfamoyl Chloride

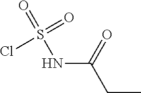

To sulfurisocyanatidic chloride (1.0 g, 7.1 mmol) was added n-propionic acid (0.5 g, 7.1 mmol) slowly dropwise in an ice-water bath. After addition was completed, toluene (10 mL) was added to the mixture. The resulting mixture was stirred for 1 h., and concentrated under reduced pressure to provide the title compound (1.1 g, crude) as a white solid which was directly used in the next step without further purification.

### Step 2: N-((4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl) sulfonyl) propionamide

To a mixture of INT B1 (20 mg, 0.44 mmol) and propionylsulfamoyl chloride (40 mg, 0.66 mmol) in MeCN (2 mL) was added TEA (10 mg, 1.2 mmol) under N₂ atmosphere. The mixture was stirred for 10 min., and concentrated. The residue was purified by Prep-HPLC on a C18 column eluting with MeCN/H₂O (30-40%, with 0.1% FA) to afford the title compound (15 mg, 48% yield) as a white solid. ¹H NMR (400 MHZ, DMSO-d₆) δ ppm 11.40 (s, 1H), 10.37 (s, 1H), 8.06 (d, J=8.6 Hz, 1H), 7.97 (s, 1H), 7.72 (d, J=8.7 Hz, 1H), 6.84 (s, 1H), 5.33 (s, 2H), 4.26 (s, 2H), 3.81 (t, J=5.7 Hz, 2H), 3.65 (d, J=11.7 Hz, 2H), 3.53 (t, J=11.6 Hz, 3H), 3.18 (s, 1H), 3.08-2.86 (m, 5H), 2.75 (d, J=11.3 Hz, 2H), 2.30 (q, J=7.5 Hz, 2H), 2.04 (q, J=7.6 Hz, 1H), 1.17 (t, J=7.5 Hz, 3H), 1.04 (t, J=7.4 Hz, 3H). LCMS calc. for C₂₈H₃₃ClF₃N₈O₆S [M+H]⁺: m/z=701.2; Found: 701.2. Example 30:4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(4-(trifluoromethyl)-1H-imidazol-5-yl)piperazine-1-carboxamide and

### Example 31: N-(2-Chloro-4-(trifluoromethyl)phenyl)-2-(2-(3,6-dihydro-2H-pyran-4-yl)-5-ethyl-7-oxo-6-(4-(4-(trifluoromethyl)-1H-imidazole-5-carbonyl)piperazin-1-yl)-[1,2,4]triazolo[1,5-a]pyrimidin-4(7H)-yl)acetamide

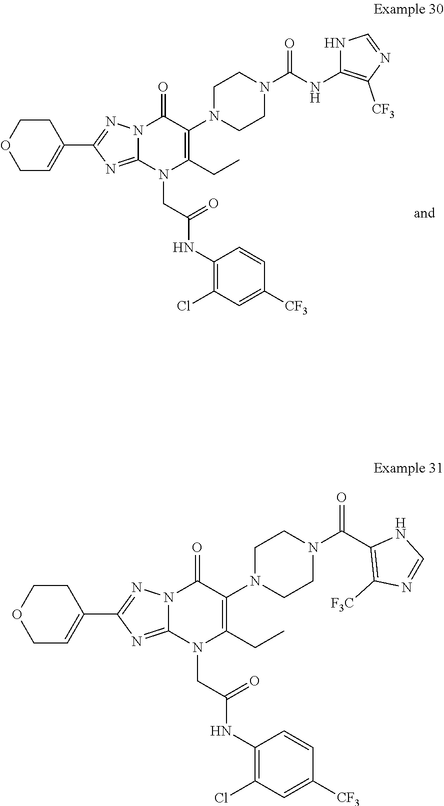 [SMILES: CCc1c(N2CCN(C(=O)Nc3[nH]cnc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3[nH]cnc3C(F)(F)F)CC2)c(=O)n2nc(C3=CCOCC3)nc2n1CC(=O)Nc1ccc(C(F)(F)F)cc1Cl]

### Step 1:4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(4-(trifluoromethyl)-1-((2-(trimethylsilyl) ethoxy)methyl)-1H-imidazol-5-yl)piperazine-1-carboxamide (Compound A) and N-(2-chloro-4-(trifluoromethyl)phenyl)-2-(2-(3,6-dihydro-2H-pyran-4-yl)-5-ethyl-7-oxo-6-(4-(4-(trifluoromethyl)-1-((2-(trimethylsilyl) ethoxy)methyl)-1H-imidazole-5-carbonyl)piperazin-1-yl)-[1,2,4]triazolo[1,5-a]pyrimidin-4(7H)-yl)acetamide (Compound B)

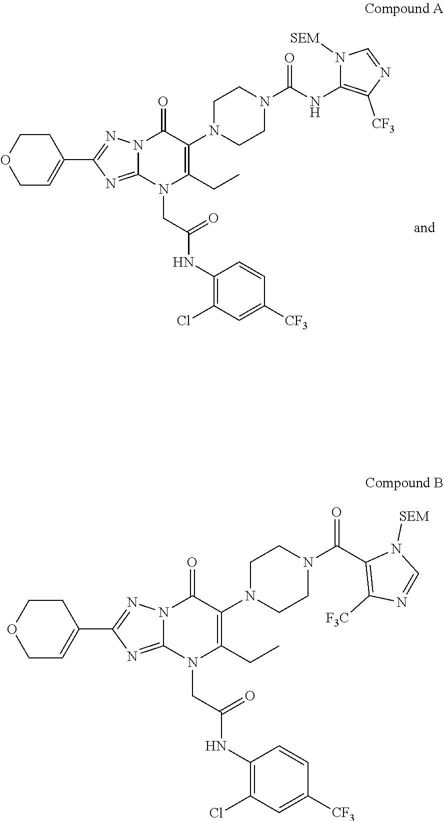 [SMILES: CCc1c(N2CCN(C(=O)Nc3c(C(F)(F)F)ncn3COCC[Si](C)(C)C)CC2)c(=O)n2nc(C3=CCOCC3)nc2n1CC(=O)Nc1ccc(C(F)(F)F)cc1Cl ; CCc1c(N2CCN(C(=O)c3c(C(F)(F)F)ncn3COCC[Si](C)(C)C)CC2)c(=O)n2nc(C3=CCOCC3)nc2n1CC(=O)Nc1ccc(C(F)(F)F)cc1Cl]

To a solution of INT C15 (50 mg, 0.16 mmol) in 1,4-dioxane (2 mL) was added TEA (25 mg, 0.24 mmol) and DPPA (66 mg, 0.24 mmol). The mixture was stirred at r.t. for 30 min. Then to the mixture was added INT B1 (50 mg, 0.08 mmol) and stirred at 90° C. for 3 h. The reaction mixture was diluted with water (30 mL) and extracted with EtOAc (10 mL×3). The combined organic layers were dried over anhydrous Na₂SO₄, filtered and concentrated. The residue was purified by flash chromatography on a silica gel column eluting with MeOH/DCM (5%) to afford the title compound A (the earlier eluted fraction, 25 mg, 36% yield) as a white solid and compound B (the latter eluted fraction, 10 mg, 15% yield) as a white solid. Compound A: LCMS calc. for C₃₆H₄₄ClF₆N₁₀O₅Si [M+H]⁺: m/z=873.3; Found: 873.0. Compound B: LCMS calc. for C₃₆H₄₃ClF₆N₉O₅Si [M+H]⁺: m/z=858.3; Found: 858.0.

### Step 2: 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(4-(trifluoromethyl)-1H-imidazol-5-yl)piperazine-1-carboxamide (Example 30)

A mixture of Compound A (25 mg, 0.03 mmol) in 4 M HCl/dioxane (2 mL) was stirred at r.t. for 2 h. The reaction mixture was evaporated under reduced pressure and the residue was purified by Prep-HPLC on a C18 column eluting with MeCN/H₂O (50-80%, with 0.1% FA) to afford the title compound (4 mg, 19% yield) as a white solid. ¹H NMR (400 MHz, DMSO-d₆) δ ppm 12.71 (s, 1H), 10.37 (s, 1H), 8.59 (s, 1H), 8.06 (d, J=8.5 Hz, 1H), 7.97 (d, J=1.7 Hz, 1H), 7.72 (dd, J=8.7, 1.6 Hz, 1H), 7.62 (s, 1H), 6.83 (s, 1H), 5.33 (s, 2H), 4.29-4.24 (m, 2H), 4.09 (d, J=12.2 Hz, 2H), 3.80 (t, J=5.4 Hz, 2H), 3.47 (t, J=10.5 Hz, 2H), 3.29 (s, 2H), 3.00 (t, J=9.2 Hz, 4H), 2.70 (d, J=10.9 Hz, 2H), 1.19 (t, J=7.5 Hz, 3H). LCMS calc. for C₃₀H₃₀ClF₆N₁₀O₄ [M+H]⁺: m/z=743.2; Found: 742.7.

### Step 3: N-(2-chloro-4-(trifluoromethyl)phenyl)-2-(2-(3,6-dihydro-2H-pyran-4-yl)-5-ethyl-7-oxo-6-(4-(4-(trifluoromethyl)-1H-imidazole-5-carbonyl)piperazin-1-yl)-[1,2,4]triazolo[1,5-a]pyrimidin-4 (7H)-yl)acetamide (Example 31)

This compound was prepared by procedures analogous to those described for Example 30 using Compound B to replace Compound A to afford the title compound as a white solid. ¹H NMR (400 MHz, DMSO-d₆) δ ppm 13.36 (s, 1H), 10.36 (s, 1H), 8.05 (d, J=8.4 Hz, 1H), 7.96 (d, J=1.7 Hz, 1H), 7.90 (s, 1H), 7.74-7.69 (m, 1H), 6.83 (s, 1H), 5.31 (s, 2H), 4.51 (s, 1H), 4.29-4.21 (m, 2H), 3.80 (t, J=5.4 Hz, 2H), 3.46 (d, J=9.9 Hz, 3H), 3.30 (s, 3H), 2.98 (d, J=7.4 Hz, 3H), 2.83-2.64 (m, 2H), 1.18 (t, J=7.4 Hz, 3H). LCMS calc. for C₃₀H₃₀ClF₆N₁₀O₄ [M+H]⁺: m/z=743.2; Found: 742.7.

### Example 32: 2-(6-(4-(5-Aminothiazole-4-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

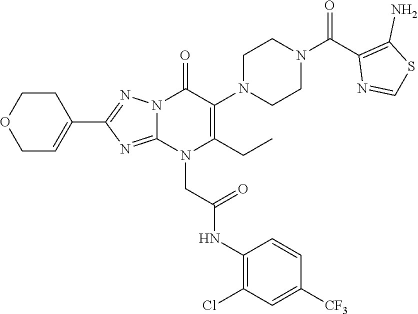 [SMILES: CCc1c(N2CCN(C(=O)c3ncsc3N)CC2)c(=O)n2nc(C3=CCOCC3)nc2n1CC(=O)Nc1ccc(C(F)(F)F)cc1Cl]

This compound was prepared by procedures analogous to those described for method A using INT B1 and 5-aminothiazole-4-carboxylic acid to afford the title compound as a white solid. ¹H NMR (400 MHZ, DMSO-d₆) δ ppm 10.37 (s, 1H), 8.06 (dd, J=8.7, 0.9 Hz, 1H), 8.01 (s, 1H), 7.98-7.96 (m, 1H), 7.74-7.69 (m, 1H), 7.29 (s, 2H), 6.83 (dd, J=3.1, 1.6 Hz, 1H), 5.32 (s, 2H), 4.25 (d, J=3.0 Hz, 2H), 3.82-3.78 (m, 2H), 3.53-3.45 (m, 2H), 3.00 (q, J=7.5 Hz, 4H), 2.72 (d, J=10.9 Hz, 4H), 1.23 (d, J=4.3 Hz, 2H), 1.22-1.16 (m, 3H); LCMS calc. for C₂₉H₃₀ClF₃N₉O₄S [M+H]⁺: m/z=692.2; Found: 692.2.

### Example 33: N-(2-Chloro-4-(trifluoromethyl)phenyl)-2-(2-(3,6-dihydro-2H-pyran-4-yl)-5-ethyl-6-(4-(N-ethylsulfamoyl)piperazin-1-yl)-7-oxo-[1,2,4]triazolo[1,5-a]pyrimidin-4(7H)-yl)acetamide

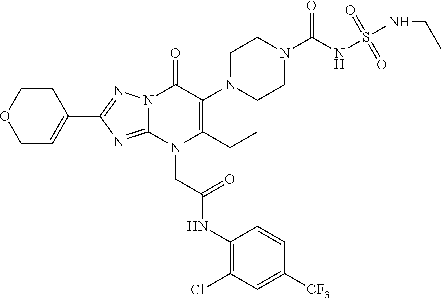 [SMILES: CCNS(=O)(=O)NC(=O)N1CCN(c2c(CC)n(CC(=O)Nc3ccc(C(F)(F)F)cc3Cl)c3nc(C4=CCOCC4)nn3c2=O)CC1]

This compound was prepared by procedures analogous to those described for Example 29 Step 2 using ethylsulfamoyl chloride to replace propionylsulfamoyl chloride to afford the title compound as a white solid. ¹H NMR (400 MHZ, DMSO-d₆)₈ ppm 10.36 (s, 1H), 8.06 (d, J=8.5 Hz, 1H), 7.97 (d, J=1.6 Hz, 1H), 7.72 (dd, J=8.7, 1.8 Hz, 1H), 7.27 (t, J=5.7 Hz, 1H), 6.83 (s, 1H), 5.32 (s, 2H), 4.25 (d, J=2.6 Hz, 2H), 3.81 (t, J=5.4 Hz, 2H), 3.51 (dd, J=24.7, 12.3 Hz, 4H), 3.02-2.89 (m, 4H), 2.79 (dd, J=24.8, 12.4 Hz, 4H), 2.51 (s, 2H), 1.18 (t, J=7.5 Hz, 3H), 1.10 (t, J=7.2 Hz, 3H). LCMS calc. for C₂₇H₃₃ClF₃N₈O₅S [M+H]⁺: m/z=673.2; Found: 673.2.

### Example 34: 4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(cyclopropylsulfonyl)piperazine-1-carboxamide

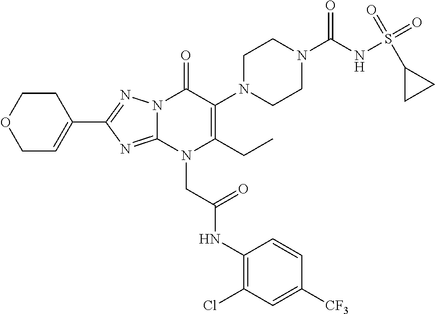 [SMILES: CCc1c(N2CCN(C(=O)NS(=O)(=O)C3CC3)CC2)c(=O)n2nc(C3=CCOCC3)nc2n1CC(=O)Nc1ccc(C(F)(F)F)cc1Cl]

### Step 1: ethyl (cyclopropylsulfonyl)carbamate

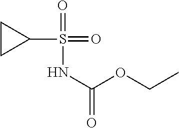

To a solution of cyclopropanesulfonamide (500 mg, 4.13 mmol) in acetone (10 mL) was added K₂CO₃ (1.71 g, 12.4 mmol) and then added ethyl carbonochloridate (669 mg, 6.2 mmol) at 0° C. The mixture was stirred at r.t. overnight, diluted with water (20 mL), extracted with EtOAc (10 mL×3).

The combined organic layers were dried over anhydrous Na₂SO₄, filtered and concentrated under reduced pressure. The residue was purified by flash chromatography on a silica gel column eluting with EtOAc/PE (0-35%) to afford the title compound (200 mg, 25% yield) as a colorless oil. LCMS calc. for C₆H₁₂NO₄S [M+H]⁺: m/z=194.0; Found: 194.0.

### Step 2: 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-N-(cyclopropylsulfonyl)piperazine-1-carboxamide

A mixture of INT B1 (20 mg, 0.035 mmol), ethy(cyclopropylsulfonyl)carbamate (27 mg, 0.14 mmol), TEA (10.7 mg, 0.11 mmol) in DME (3 mL) was stirred at 120° C. overnight. The mixture was concentrated and the residue was purified by Prep-HPLC on a C18 column eluting with MeCN/H₂O (10-95%, with 0.1% NH₄HCO₃) to afford the title compound (7 mg, 28%) as a white solid. ¹H NMR (400 MHZ, DMSO-d₆) δ ppm 10.53 (s, 1H), 10.36 (s, 1H), 8.06 (d, J=8.5 Hz, 1H), 7.97 (d, J=1.6 Hz, 1H), 7.78-7.66 (m, 1H), 6.83 (s, 1H), 5.32 (s, 2H), 4.25 (d, J=2.6 Hz, 2H), 4.01 (d, J=12.0 Hz, 2H), 3.80 (t, J=5.4 Hz, 2H), 3.42 (t, J=10.5 Hz, 2H), 3.32 (s, 2H), 3.12 (t, J=6.2 Hz, 1H), 2.96 (t, J=7.4 Hz, 4H), 2.68 (d, J=11.4 Hz, 2H), 1.18 (t, J=7.5 Hz, 3H), 1.06 (d, J=4.2 Hz, 4H). LCMS calc. for C₂₉H₃₃ClF₃N₈O₆S [M+H]⁺: m/z=713.2; Found: 713.2.

### Example 35: N-(2-Chloro-4-(trifluoromethyl)phenyl)-N-(2-(2-(3,6-dihydro-2H-pyran-4-yl)-5-ethyl-7-oxo-6-(4-sulfamoylpiperazin-1-yl)-[1,2,4]triazolo[1,5-a]pyrimidin-4(7H)-yl)acetyl)propionamide

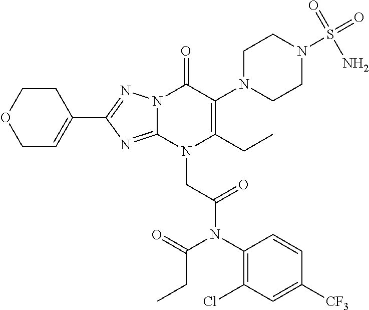 [SMILES: CCC(=O)N(C(=O)Cn1c(CC)c(N2CCN(S(N)(=O)=O)CC2)c(=O)n2nc(C3=CCOCC3)nc12)c1ccc(C(F)(F)F)cc1Cl]

To a solution of N-(2-chloro-4-(trifluoromethyl)phenyl)-2-(2-(3,6-dihydro-2H-pyran-4-yl)-5-ethyl-7-oxo-6-(4-sulfamoylpiperazin-1-yl)-[1,2,4]triazolo[1,5-a]pyrimidin-4(7H)-yl)acetamide (20 mg, 0.031 mmol, Example 28) and propionyl chloride (4.0 mg, 0.04 mmol) in DCM (3 mL) was added TEA (6.3 mg, 0.062 mmol). The mixture was stirred at r.t. overnight and concentrated under reduced pressure. The residue was purified by Prep-HPLC on a C18 column eluting with MeCN/H₂O (15-50%, with 0.1% FA) to give title compound (4 mg, 18.4% yield) as a white solid. ¹H NMR (400 MHZ, DMSO-d₆) δ ppm 8.17 (d, J=1.5 Hz, 1H), 7.97˜7.84 (m, 2H), 6.84 (s, 2H), 5.33 (t, J=4.6 Hz, 1H), 4.33˜4.22 (m, 2H), 3.81 (p, J=6.1 Hz, 2H), 3.60˜3.49 (m, 2H), 3.42 (d, J=7.7 Hz, 2H), 2.95 (s, 1H), 2.82 (s, 1H), 2.77˜2.63 (m, 4H), 2.39˜2.30 (m, 1H), 2.18˜2.09 (m, 1H), 2.08˜1.85 (m, 2H), 1.16 (t, J=7.5 Hz, 3H), 1.02 (t, J=7.1 Hz, 3H). LCMS calc. for C₂₈H₃₃ClF₃N₈O₆S [M+H]⁺: m/z=701.2; Found: 701.2.

### Example 36: 4-(8-(2-((2-Chloro-4-(trifluoromethyl)phenyl)amino)-2-oxoethyl)-2-(3,6-dihydro-2H-pyran-4-yl)-7-ethyl-5-oxo-5,8-dihydroimidazo[1,2-a]pyrimidin-6-yl)-N-(5-methoxypyrimidin-4-yl)piperazine-1-carboxamide

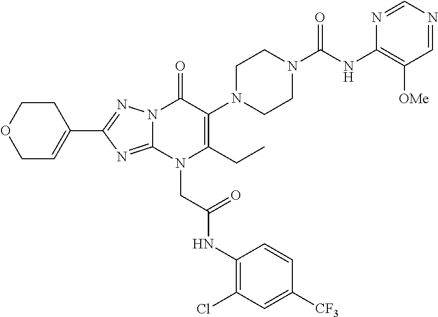 [SMILES: CCc1c(N2CCN(C(=O)Nc3ncncc3OC)CC2)c(=O)n2nc(C3=CCOCC3)nc2n1CC(=O)Nc1ccc(C(F)(F)F)cc1Cl]

### Step 1:4-nitrophenyl 4-(8-(2-((2-chloro-4-(trifluoromethyl)phenyl)amino)-2-oxoethyl)-2-(3,6-dihydro-2H-pyran-4-yl)-7-ethyl-5-oxo-5,8-dihydroimidazo[1,2-a]pyrimidin-6-yl)piperazine-1-carboxylate

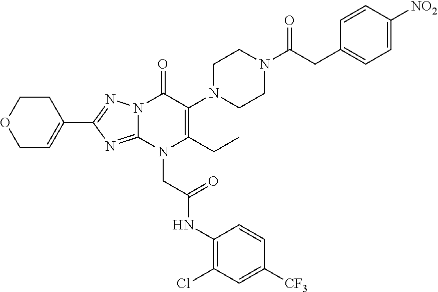 [SMILES: CCc1c(N2CCN(C(=O)Cc3ccc([N+](=O)[O-])cc3)CC2)c(=O)n2nc(C3=CCOCC3)nc2n1CC(=O)Nc1ccc(C(F)(F)F)cc1Cl]

To a solution of INT B1 (100 mg, 0.18 mmol) in THF (2 mL) was added TEA (0.05 mL, 0.36 mmol) and 4-nitrophenyl carbonochloridate (71 mg, 0.36 mmol) dropwise in an ice-water bath under nitrogen atmosphere. The mixture was stirred at r.t for 30 min. The reaction mixture was concentrated under reduced pressure. The residue was purified by flash chromatography on a C18 column eluting with MeCN/H₂O (55-85%, with 0.05% NH₄HCO₃) to give the title compound (65 mg, 49% yield) as a yellow solid.

### Step 2: 4-(8-(2-((2-chloro-4-(trifluoromethyl)phenyl)amino)-2-oxoethyl)-2-(3,6-dihydro-2H-pyran-4-yl)-7-ethyl-5-oxo-5,8-dihydroimidazo[1,2-a]pyrimidin-6-yl)-N-(5-methoxypyrimidin-4-yl)piperazine-1-carboxamide

To a solution of 5-methoxypyrimidin-4-amine (9 mg, 0.07 mol) in DMF (2 mL) was added NaH (60% dispersion in mineral oil, 6 mg, 0.14 mmol) in an ice-water bath. The mixture was stirred for 30 min. Then 4-nitrophenyl 4-(8-(2-((2-chloro-4-(trifluoromethyl)phenyl)amino)-2-oxoethyl)-2-(3,6-dihydro-2H-pyran-4-yl)-7-ethyl-5-oxo-5,8-dihydroimidazo[1,2-a]pyrimidin-6-yl)piperazine-1-carboxylate (65 mg, 0.09 mmol) was added. The mixture was stirred at r.t. for 1 h., and concentrated. The residue was purified by flash chromatography on a C18 column eluting with MeCN/H₂O (5%-95%, with 0.1% FA) to obtain the title compound (6.37 mg, 12% yield) as a white solid. ¹H NMR (400 MHz, DMSO-d₆) δ ppm 10.38 (s, 1H), 9.05 (s, 1H), 8.39 (s, 1H), 8.27 (s, 1H), 8.06 (d, J=8.5 Hz, 1H), 7.97 (s, 1H), 7.72 (d, J=7.2 Hz, 1H), 6.84 (s, 1H), 5.32 (s, 2H), 4.26 (d, J=2.3 Hz, 2H), 4.06 (d, J=12.6 Hz, 2H), 3.90 (s, 3H), 3.81 (t, J=5.3 Hz, 2H), 3.56-3.45 (m, 5H), 3.02 (dd, J=26.3, 9.6 Hz, 4H), 2.68 (d, J=10.0 Hz, 2H), 1.20 (t, J=7.4 Hz, 3H). LCMS calc. for C₃₂H₃₃ClF₃N₉O₅ [M+H]⁺: m/z=717.2; Found: 717.3.

### Example 37: tert-Butyl (2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)carbamate

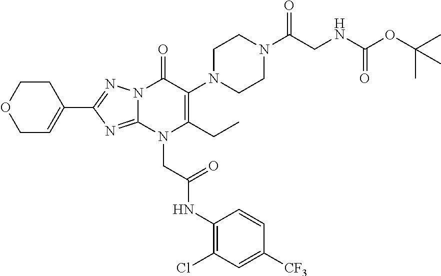 [SMILES: CCc1c(N2CCN(C(=O)CNC(=O)OC(C)(C)C)CC2)c(=O)n2nc(C3=CCOCC3)nc2n1CC(=O)Nc1ccc(C(F)(F)F)cc1Cl]

To a solution of INT B1 (10.0 mg, 0.02 mmol) in DCM (2 mL) was added (tert-butoxycarbonyl)glycine (2.6 mg, 0.02 mmol), HOBt (3.4 mg, 0.02 mmol), DMAP (1.8 mg, 0.02 mmol) and EDCI (3.4 mg, 0.02 mmol) at 0° C. The mixture was stirred at 0° C. for 2 h. The reaction mixture was concentrated under reduced pressure. The residue was purified by Prep-HPLC on a C₁₈ column eluting with MeCN/H₂O (60-75% with 0.05% NH₄HCO₃) to afford the title compound (3.53 mg, 33% yield) as a white solid. ¹H NMR (400 MHZ, DMSO-d₆) δ ppm 10.30 (s, 1H), 8.06 (d, J=8.5 Hz, 1H), 7.97 (s, 1H), 7.72 (d, J=8.6 Hz, 1H), 6.83 (s, 1H), 6.77 (t, J=5.9 Hz, 1H), 5.32 (s, 2H), 4.36 (d, J=11.3 Hz, 1H), 4.25 (d, J=2.1 Hz, 2H), 3.95-3.76 (m, 5H), 3.52-3.34 (m, 3H), 3.19 (d, J=10.9 Hz, 1H), 2.98 (d, J=7.3 Hz, 2H), 2.77 (d, J=11.1 Hz, 1H), 2.69 (d, J=12.9 Hz, 2H), 1.40 (s, 9H), 1.19 (t, J=7.5 Hz, 4H). LCMS calc. for C₃₂H₃₉ClF₃N₈₀₆ [M+H]⁺: m/z=723.3; Found: [M+H−100]⁺: 623.2.

### Example 38: N-(2-Chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide

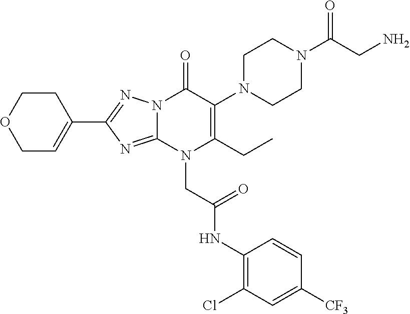 [SMILES: CCc1c(N2CCN(C(=O)CN)CC2)c(=O)n2nc(C3=CCOCC3)nc2n1CC(=O)Nc1ccc(C(F)(F)F)cc1Cl]

A mixture of tert-butyl (2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)carbamate (15 mg, 0.02 mmol, Example 37) in 4 M HCl/1,4-dioxane (5 mL) was stirred at r.t. for 1 h. The reaction mixture was concentrated under reduced pressure. The residue was purified by Prep-HPLC on a C18 column eluting with MeCN/H₂O (55-70%, with 0.05% NH₄HCO₃) to afford the title compound (9.6 mg, 74% yield) as a white solid. ¹H NMR (400 MHZ, DMSO-d₆) δ ppm 8.06 (d, J=8.5 Hz, 1H), 7.96 (s, 1H), 7.71 (d, J=7.3 Hz, 1H), 6.83 (s, 1H), 5.31 (s, 2H), 4.41 (d, J=11.7 Hz, 1H), 4.25 (d, J=2.4 Hz, 2H), 3.90-3.70 (m, 4H), 3.45 (s, 3H), 3.17 (d, J=12.3 Hz, 2H), 2.98 (d, J=7.3 Hz, 2H), 2.78 (d, J=14.5 Hz, 4H), 1.20 (dd, J=17.3, 10.0 Hz, 5H). LCMS calc. for C₂₇H₃₁ClF₃N₈O₄ [M+H]⁺: m/z=623.2; Found: 623.2.

### Example 39: N-(2-Chloro-4-(trifluoromethyl)phenyl)-2-(2-(3,6-dihydro-2H-pyran-4-yl)-5-ethyl-6-(4-((ethylsulfonyl)glycyl)piperazin-1-yl)-7-oxo-[1,2,4]triazolo[1,5-a]pyrimidin-4(7H)-yl)acetamide

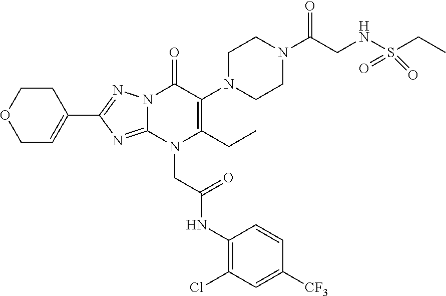 [SMILES: CCc1c(N2CCN(C(=O)CNS(=O)(=O)CC)CC2)c(=O)n2nc(C3=CCOCC3)nc2n1CC(=O)Nc1ccc(C(F)(F)F)cc1Cl]

To a solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (25 mg, 0.04 mmol, Example 38) in DCM (2 mL) was added TEA (16.2 mg, 0.16 mmol) and ethanesulfonyl chloride (5.1 mg, 0.04 mmol) at 0° C. The resulting mixture was stirred at 0° C. for 2 h. and concentrated. The residue was purified by Prep-HPLC on a C18 column eluting with MeCN/H₂O (60-75%, with 0.05% NH₄HCO₃) to afford the title compound (14.7 mg, 51% yield) as a white solid. ¹H NMR (400 MHZ, DMSO-d₆) δ ppm 10.36 (s, 1H), 8.06 (d, J=8.5 Hz, 1H), 7.97 (s, 1H), 7.79-7.66 (m, 1H), 7.13 (t, J=5.4 Hz, 1H), 6.83 (s, 1H), 5.32 (s, 2H), 4.38 (d, J=12.2 Hz, 1H), 4.25 (d, J=2.3 Hz, 2H), 4.07-3.99 (m, 1H), 3.85 (ddd, J=17.5, 13.8, 5.4 Hz, 4H), 3.38 (d, J=11.6 Hz, 3H), 3.03 (dt, J=22.8, 7.4 Hz, 4H), 2.79 (t, J=12.4 Hz, 4H), 1.24 (t, J=7.3 Hz, 4H), 1.19 (t, J=7.5 Hz, 3H). LCMS calc. for C₂₉H₃₅ClF₃N₈O₆S [M+H]⁺: m/z=715.2; Found: 715.0.

### Example 40: Ethyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

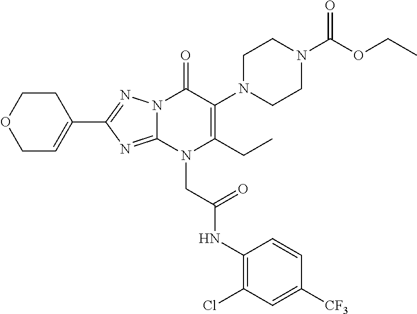 [SMILES: CCOC(=O)N1CCN(c2c(CC)n(CC(=O)Nc3ccc(C(F)(F)F)cc3Cl)c3nc(C4=CCOCC4)nn3c2=O)CC1]

To a solution of INT B1 (20.0 mg, 0.04 mmol) in DCM (2 mL) was added TEA (5.4 mg, 0.11 mmol) and ethyl carbonochloridate (15 mg, 0.14 mmol) at 0° C. The mixture was stirred at 0° C. for 2 h. and concentrated. The residue was purified by Prep-HPLC on a C18 column eluting with MeCN/H₂O (60-75%, with 0.05% NH₄HCO₃) to afford the title compound (8.1 mg, 36% yield) as a white solid. ¹H NMR (400 MHz, DMSO-d₆) δ ppm 10.38 (s, 1H), 8.06 (d, J=8.6 Hz, 1H), 7.97 (d, J=1.6 Hz, 1H), 7.72 (d, J=8.8 Hz, 1H), 6.83 (s, 1H), 5.32 (s, 2H), 4.25 (d, J=2.5 Hz, 2H), 4.08 (q, J=7.1 Hz, 2H), 3.98 (d, J=11.9 Hz, 2H), 3.80 (t, J=5.4 Hz, 2H), 3.45-3.36 (m, 3H), 2.97 (d, J=7.4 Hz, 4H), 2.67 (d, J=10.8 Hz, 2H), 1.28-1.12 (m, 7H). LCMS calc. for C₂₈H₃₂ClF₃N₇₀₅ [M+H]⁺: m/z=638.2; Found: 638.2.

### Example 41: N-(2-Chloro-4-(trifluoromethyl)phenyl)-2-(2-(3,6-dihydro-2H-pyran-4-yl)-5-ethyl-6-(4-((ethylcarbamoyl)glycyl)piperazin-1-yl)-7-oxo-[1,2,4]triazolo[1,5-a]pyrimidin-4(7H)-yl)acetamide

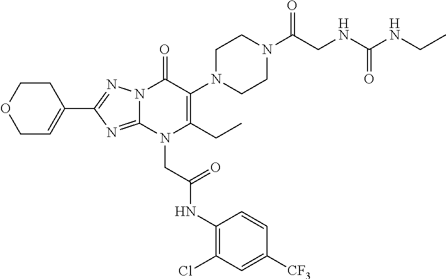 [SMILES: CCNC(=O)NCC(=O)N1CCN(c2c(CC)n(CC(=O)Nc3ccc(C(F)(F)F)cc3Cl)c3nc(C4=CCOCC4)nn3c2=O)CC1]

To a stirred solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.08 mmol, Example 38) in DCM (2 mL) was added isocyanatoethane (10 mg, 0.14 mmol) at −78° C., the mixture was stirred at −78° C. for 1 h. And the reaction mixture was concentrated under reduced pressure. The residue was purified by Prep-HPLC on a C18 column eluting with MeCN/H₂O (45-70%, with 0.05% FA) to afford the title compound (25 mg, 45% yield) as a white solid. ¹H NMR (400 MHZ, DMSO-d₆)₈ ppm 10.36 (s, 1H), 8.06 (d, J=8.6 Hz, 1H), 7.97 (d, J=2.1 Hz, 1H), 7.72 (dd, J=8.9, 2.1 Hz, 1H), 6.86-6.80 (m, 1H), 6.23 (t, J=5.6 Hz, 1H), 5.94 (t, J=5.0 Hz, 1H), 5.32 (s, 2H), 4.38 (d, J=12.3 Hz, 1H), 4.25 (q, J=2.8 Hz, 2H), 3.96 (d, J=4.9 Hz, 1H), 3.90 (d, J=5.1 Hz, 1H), 3.80 (t, J=5.5 Hz, 3H), 3.45 (t, J=11.9 Hz, 2H), 3.36 (d, J=3.4 Hz, 2H), 3.19 (t, J=12.0 Hz, 2H), 3.04-2.96 (m, 4H), 2.81-2.76 (m, 1H), 2.70 (d, J=11.4 Hz, 2H), 1.19 (t, J=7.5 Hz, 3H), 0.99 (t, J=7.2 Hz, 3H). LCMS calc. for C₃₀H₃₆ClF₃N₉O₅ [M+H]⁺: m/z=694.2; Found: 694.2. Example 42: N-(2-Chloro-4-(trifluoromethyl)phenyl)-2-(2-(3,6-dihydro-2H-pyran-4-yl)-5-ethyl-6-(4-(4-hydroxy-1H-pyrrolo[2,3-b]pyridine-5-carbonyl)piperazin-1-yl)-7-oxo-[1,2,4]triazolo[1,5-a]pyrimidin-4(7H)-yl)acetamide

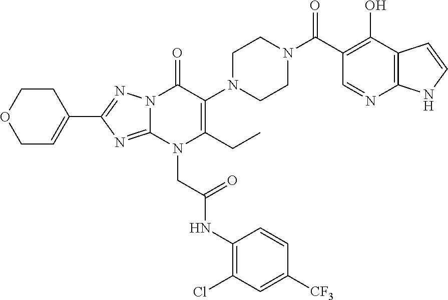 [SMILES: CCc1c(N2CCN(C(=O)c3cnc4[nH]ccc4c3O)CC2)c(=O)n2nc(C3=CCOCC3)nc2n1CC(=O)Nc1ccc(C(F)(F)F)cc1Cl]

Method B: To a solution of INT B1 (15.0 mg, 0.03 mmol) and INT C6 (5.67 mg, 0.03 mmol) in MeCN (4 mL) was added HATU (15 mg, 0.04 mmol) and K₂CO₃ (11.3 mg, 0.08 mmol). The mixture was stirred at r.t. for 2 h. And the reaction mixture was concentrated under reduced pressure. The residue was adjusted to pH to 6-7 with HCl aq. (1 N), and purified by Prep-HPLC on a C18 column eluting with MeCN/H₂O (15-55%, with 0.1% FA) to afford the title compound (8.61 mg, 45% yield) as a white solid. ¹H NMR (400 MHZ, DMSO-d₆) δ ppm 11.61 (s, 1H), 10.93 (s, 1H), 10.36 (s, 1H), 8.06 (d, J=8.3 Hz, 1H), 7.96 (d, J=6.1 Hz, 2H), 7.72 (d, J=8.3 Hz, 1H), 7.27 (s, 1H), 6.83 (s, 1H), 6.69 (s, 1H), 5.32 (s, 2H), 4.26 (s, 2H), 3.81 (d, J=5.2 Hz, 2H), 3.49 (s, 4H), 3.15-3.09 (m, 2H), 2.99 (d, J=7.1 Hz, 2H), 2.72 (s, 3H), 1.18 (dd, J=13.3, 7.0 Hz, 4H). LCMS calc. for C₃₃H₃₂ClF₃N₉O₅ [M+H]⁺: m/z=726.2; Found: 726.2.

The following compounds listed in Table 1 were prepared by using an appropriate acid and INT

B1 as the methods analogous to those described for method B.

TABLE 1
Preparations of Examples (Ex)

| Ex | Acid | Structure |
|---|---|---|
| 43 | 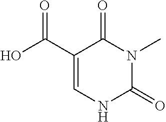 | 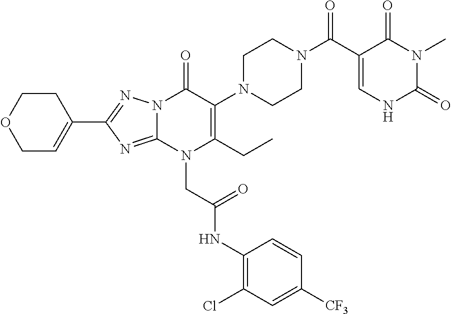 [SMILES: CCc1c(N2CCN(C(=O)c3c[nH]c(=O)n(C)c3=O)CC2)c(=O)n2nc(C3=CCOCC3)nc2n1CC(=O)Nc1ccc(C(F)(F)F)cc1Cl] |
| 44 | INT C13 | 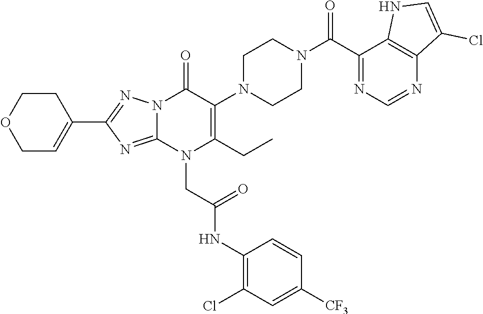 [SMILES: CCc1c(N2CCN(C(=O)c3ncnc4c(Cl)c[nH]c34)CC2)c(=O)n2nc(C3=CCOCC3)nc2n1CC(=O)Nc1ccc(C(F)(F)F)cc1Cl] |
| 45 | 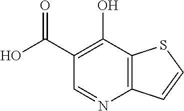 [SMILES: O=C(O)c1cnc2ccsc2c1O] | 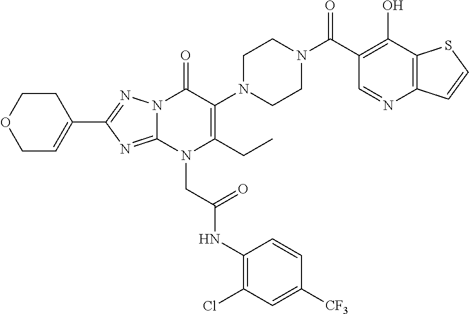 [SMILES: CCc1c(N2CCN(C(=O)c3cnc4ccsc4c3O)CC2)c(=O)n2nc(C3=CCOCC3)nc2n1CC(=O)Nc1ccc(C(F)(F)F)cc1Cl] |
| 46 | 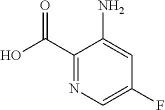 [SMILES: Nc1cc(F)cnc1C(=O)O] | 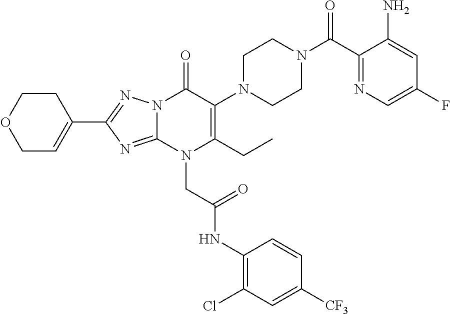 [SMILES: CCc1c(N2CCN(C(=O)c3ncc(F)cc3N)CC2)c(=O)n2nc(C3=CCOCC3)nc2n1CC(=O)Nc1ccc(C(F)(F)F)cc1Cl] |
| 47 | 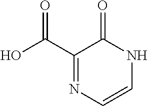 | 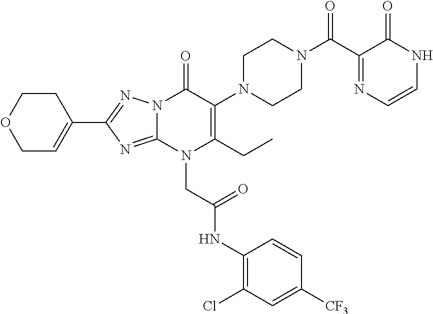 [SMILES: CCc1c(N2CCN(C(=O)c3ncc[nH]c3=O)CC2)c(=O)n2nc(C3=CCOCC3)nc2n1CC(=O)Nc1ccc(C(F)(F)F)cc1Cl] |
| 48 | 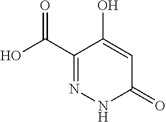 [SMILES: O=C(O)c1n[nH]c(=O)cc1O] | 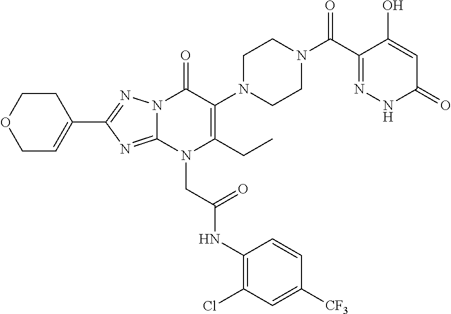 [SMILES: CCc1c(N2CCN(C(=O)c3n[nH]c(=O)cc3O)CC2)c(=O)n2nc(C3=CCOCC3)nc2n1CC(=O)Nc1ccc(C(F)(F)F)cc1Cl] |
| 49 | INT C17 | 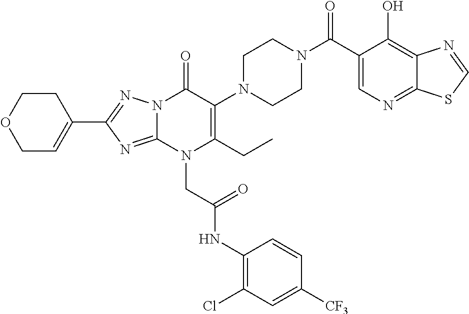 [SMILES: CCc1c(N2CCN(C(=O)c3cnc4scnc4c3O)CC2)c(=O)n2nc(C3=CCOCC3)nc2n1CC(=O)Nc1ccc(C(F)(F)F)cc1Cl] |
| 50 | 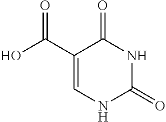 [SMILES: O=C(O)c1c[nH]c(=O)[nH]c1=O] | 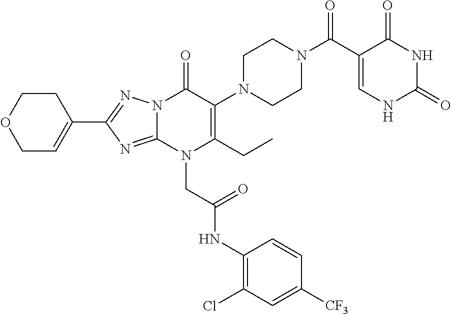 [SMILES: CCc1c(N2CCN(C(=O)c3c[nH]c(=O)[nH]c3=O)CC2)c(=O)n2nc(C3=CCOCC3)nc2n1CC(=O)Nc1ccc(C(F)(F)F)cc1Cl] |
| 51 | 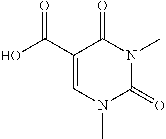 | 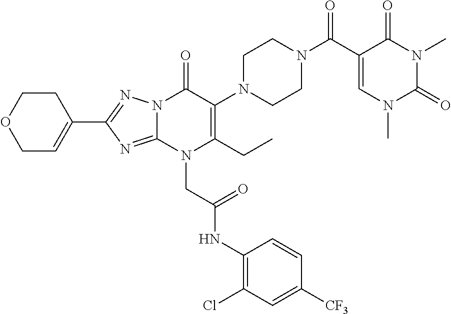 [SMILES: CCc1c(N2CCN(C(=O)c3cn(C)c(=O)n(C)c3=O)CC2)c(=O)n2nc(C3=CCOCC3)nc2n1CC(=O)Nc1ccc(C(F)(F)F)cc1Cl] |
| 52 | INT C7 | 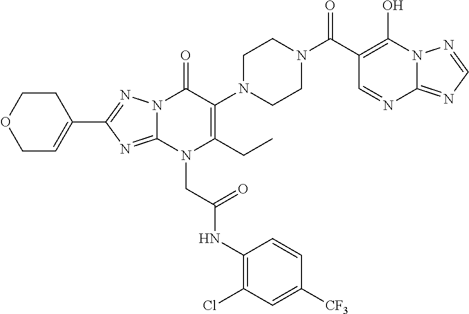 [SMILES: CCc1c(N2CCN(C(=O)c3cnc4ncnn4c3O)CC2)c(=O)n2nc(C3=CCOCC3)nc2n1CC(=O)Nc1ccc(C(F)(F)F)cc1Cl] |
| 53 | 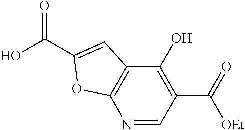 [SMILES: CCOC(=O)c1cnc2oc(C(=O)O)cc2c1O] | 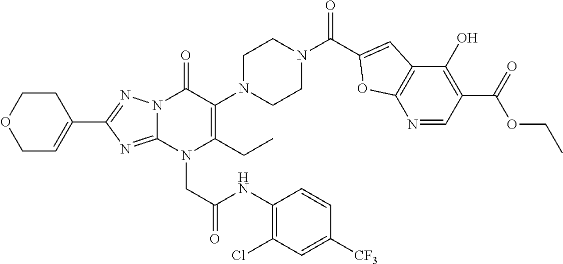 [SMILES: CCOC(=O)c1cnc2oc(C(=O)N3CCN(c4c(CC)n(CC(=O)Nc5ccc(C(F)(F)F)cc5Cl)c5nc(C6=CCOCC6)nn5c4=O)CC3)cc2c1O] |
| 54 | INT C20 | 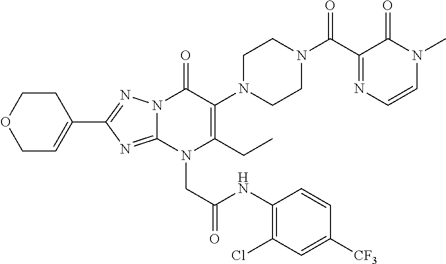 [SMILES: CCc1c(N2CCN(C(=O)c3nccn(C)c3=O)CC2)c(=O)n2nc(C3=CCOCC3)nc2n1CC(=O)Nc1ccc(C(F)(F)F)cc1Cl] |
| 55 | INT C8 | 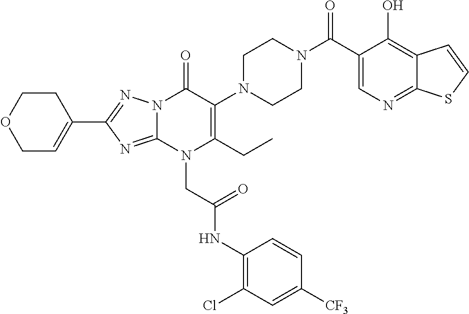 [SMILES: CCc1c(N2CCN(C(=O)c3cnc4sccc4c3O)CC2)c(=O)n2nc(C3=CCOCC3)nc2n1CC(=O)Nc1ccc(C(F)(F)F)cc1Cl] |
| 56 | INT C10 | 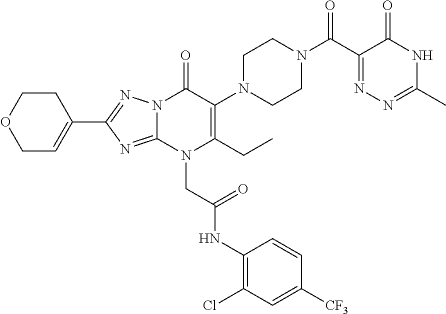 [SMILES: CCc1c(N2CCN(C(=O)c3nnc(C)[nH]c3=O)CC2)c(=O)n2nc(C3=CCOCC3)nc2n1CC(=O)Nc1ccc(C(F)(F)F)cc1Cl] |
| 57 | INT C22 | 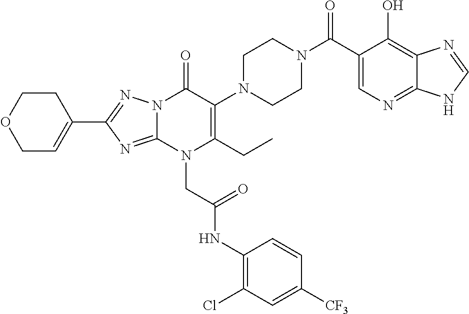 [SMILES: CCc1c(N2CCN(C(=O)c3cnc4[nH]cnc4c3O)CC2)c(=O)n2nc(C3=CCOCC3)nc2n1CC(=O)Nc1ccc(C(F)(F)F)cc1Cl] |
| 58 | INT C23 | 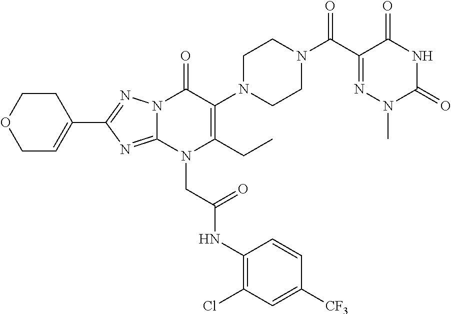 [SMILES: CCc1c(N2CCN(C(=O)c3nn(C)c(=O)[nH]c3=O)CC2)c(=O)n2nc(C3=CCOCC3)nc2n1CC(=O)Nc1ccc(C(F)(F)F)cc1Cl] |
| 59 | INT C24 | 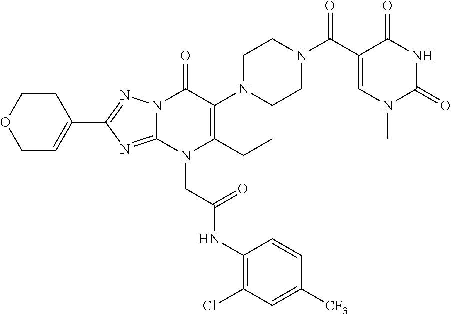 [SMILES: CCc1c(N2CCN(C(=O)c3cn(C)c(=O)[nH]c3=O)CC2)c(=O)n2nc(C3=CCOCC3)nc2n1CC(=O)Nc1ccc(C(F)(F)F)cc1Cl] |
| 60 | 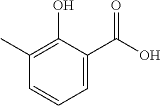 | 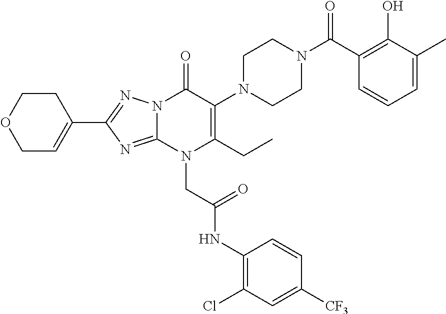 [SMILES: CCc1c(N2CCN(C(=O)c3cccc(C)c3O)CC2)c(=O)n2nc(C3=CCOCC3)nc2n1CC(=O)Nc1ccc(C(F)(F)F)cc1Cl] |
| 61 | 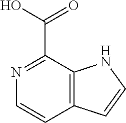 [SMILES: O=C(O)c1nccc2cc[nH]c12] | 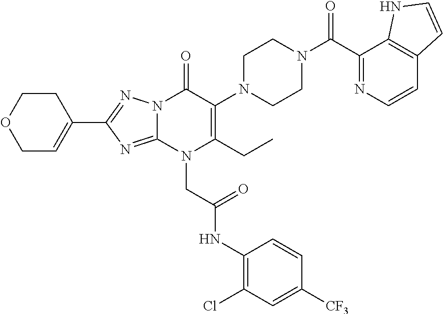 [SMILES: CCc1c(N2CCN(C(=O)c3nccc4cc[nH]c34)CC2)c(=O)n2nc(C3=CCOCC3)nc2n1CC(=O)Nc1ccc(C(F)(F)F)cc1Cl] |
| 62 | 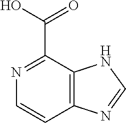 [SMILES: O=C(O)c1nccc2nc[nH]c12] | 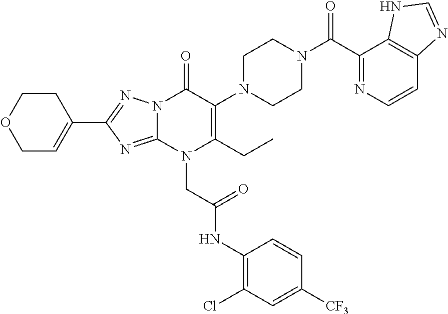 [SMILES: CCc1c(N2CCN(C(=O)c3nccc4nc[nH]c34)CC2)c(=O)n2nc(C3=CCOCC3)nc2n1CC(=O)Nc1ccc(C(F)(F)F)cc1Cl] |
| 63 | INT C25 | 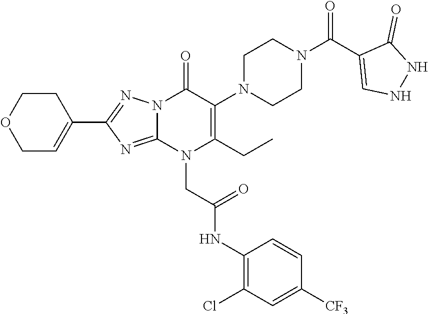 [SMILES: CCc1c(N2CCN(C(=O)c3c[nH][nH]c3=O)CC2)c(=O)n2nc(C3=CCOCC3)nc2n1CC(=O)Nc1ccc(C(F)(F)F)cc1Cl] |
| 64 | INT C26 | 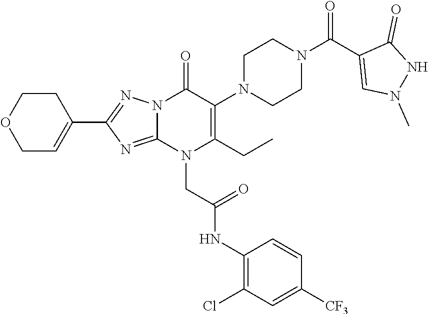 [SMILES: CCc1c(N2CCN(C(=O)c3cn(C)[nH]c3=O)CC2)c(=O)n2nc(C3=CCOCC3)nc2n1CC(=O)Nc1ccc(C(F)(F)F)cc1Cl] |
| 65 | 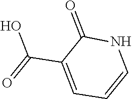 | 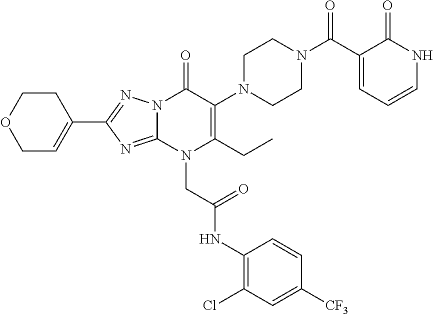 [SMILES: CCc1c(N2CCN(C(=O)c3ccc[nH]c3=O)CC2)c(=O)n2nc(C3=CCOCC3)nc2n1CC(=O)Nc1ccc(C(F)(F)F)cc1Cl] |
| 66 | 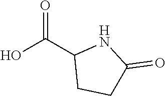 | 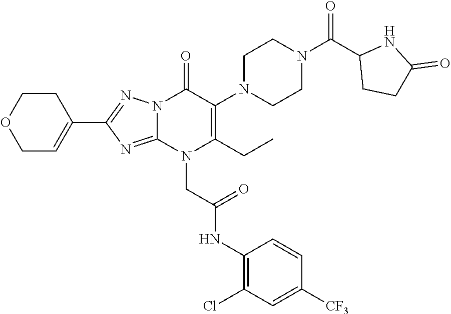 [SMILES: CCc1c(N2CCN(C(=O)C3CCC(=O)N3)CC2)c(=O)n2nc(C3=CCOCC3)nc2n1CC(=O)Nc1ccc(C(F)(F)F)cc1Cl] |
| 67 | 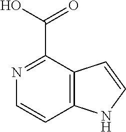 [SMILES: O=C(O)c1nccc2[nH]ccc12] | 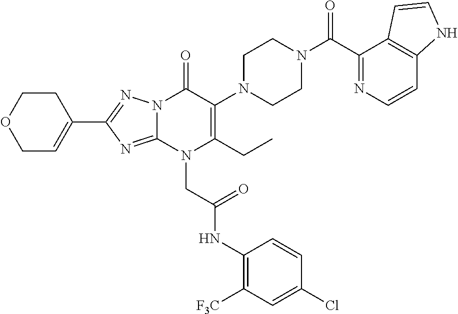 [SMILES: CCc1c(N2CCN(C(=O)c3nccc4[nH]ccc34)CC2)c(=O)n2nc(C3=CCOCC3)nc2n1CC(=O)Nc1ccc(Cl)cc1C(F)(F)F] |
| 68 | 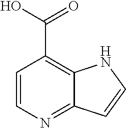 [SMILES: O=C(O)c1ccnc2cc[nH]c12] | 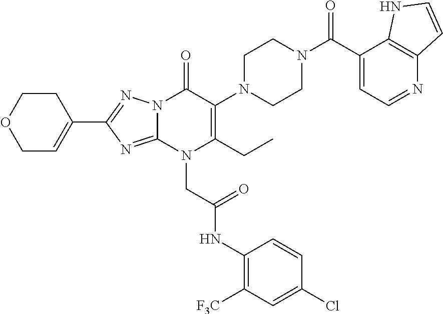 [SMILES: CCc1c(N2CCN(C(=O)c3ccnc4cc[nH]c34)CC2)c(=O)n2nc(C3=CCOCC3)nc2n1CC(=O)Nc1ccc(Cl)cc1C(F)(F)F] |
| 69 | 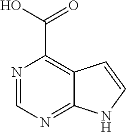 [SMILES: O=C(O)c1ncnc2[nH]ccc12] | 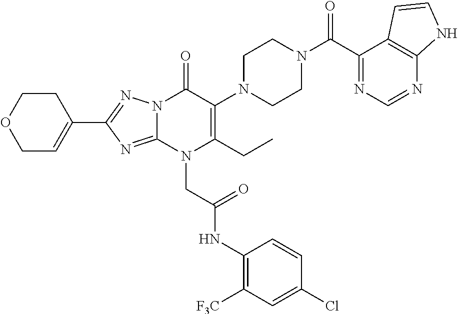 [SMILES: CCc1c(N2CCN(C(=O)c3ncnc4[nH]ccc34)CC2)c(=O)n2nc(C3=CCOCC3)nc2n1CC(=O)Nc1ccc(Cl)cc1C(F)(F)F] |
| 70 | 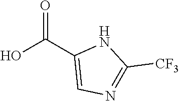 [SMILES: O=C(O)c1cnc(C(F)(F)F)[nH]1] | 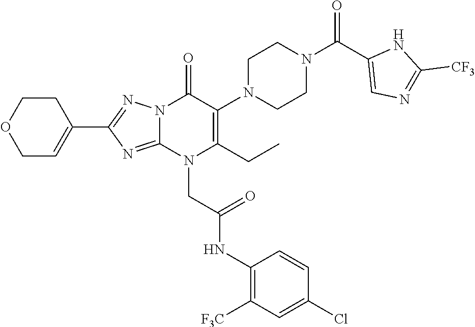 [SMILES: CCc1c(N2CCN(C(=O)c3cnc(C(F)(F)F)[nH]3)CC2)c(=O)n2nc(C3=CCOCC3)nc2n1CC(=O)Nc1ccc(Cl)cc1C(F)(F)F] |
| 71 | 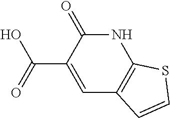 [SMILES: O=C(O)c1cc2ccsc2[nH]c1=O] | 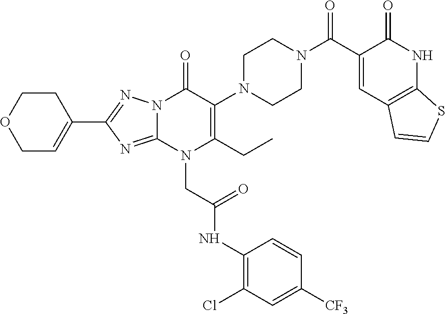 [SMILES: CCc1c(N2CCN(C(=O)c3cc4ccsc4[nH]c3=O)CC2)c(=O)n2nc(C3=CCOCC3)nc2n1CC(=O)Nc1ccc(C(F)(F)F)cc1Cl] |
| 72 | INT C38 | 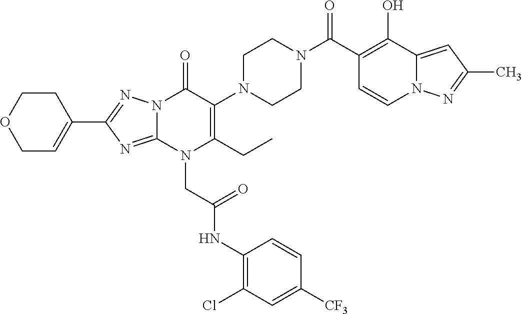 [SMILES: CCc1c(N2CCN(C(=O)c3ccn4nc(C)cc4c3O)CC2)c(=O)n2nc(C3=CCOCC3)nc2n1CC(=O)Nc1ccc(C(F)(F)F)cc1Cl] |
| 73 | 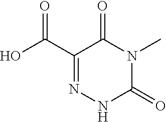 | 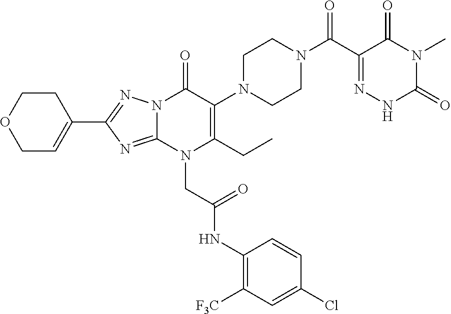 [SMILES: CCc1c(N2CCN(C(=O)c3n[nH]c(=O)n(C)c3=O)CC2)c(=O)n2nc(C3=CCOCC3)nc2n1CC(=O)Nc1ccc(Cl)cc1C(F)(F)F] |
| 74 | INT C35 | 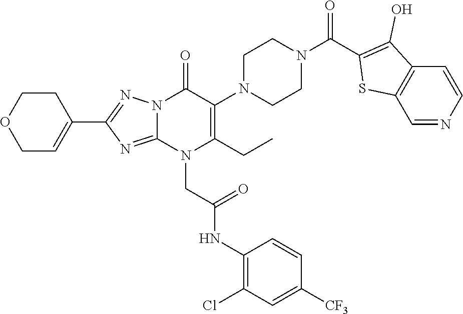 [SMILES: CCc1c(N2CCN(C(=O)c3sc4cnccc4c3O)CC2)c(=O)n2nc(C3=CCOCC3)nc2n1CC(=O)Nc1ccc(C(F)(F)F)cc1Cl] |
| 75 | INT C36 | 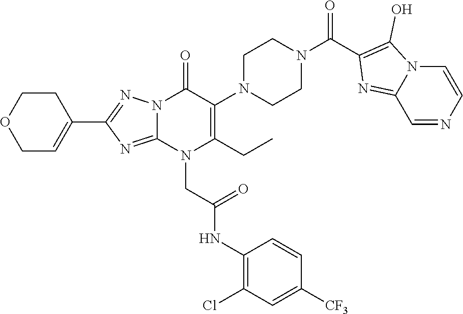 [SMILES: CCc1c(N2CCN(C(=O)c3nc4cnccn4c3O)CC2)c(=O)n2nc(C3=CCOCC3)nc2n1CC(=O)Nc1ccc(C(F)(F)F)cc1Cl] |
| 76 | INT C37 | 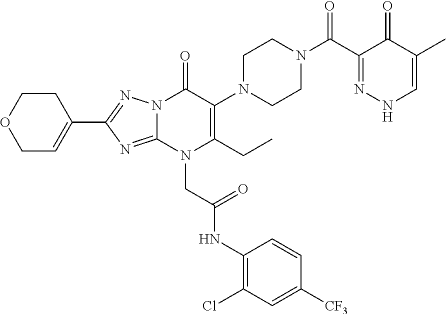 [SMILES: CCc1c(N2CCN(C(=O)c3n[nH]cc(C)c3=O)CC2)c(=O)n2nc(C3=CCOCC3)nc2n1CC(=O)Nc1ccc(C(F)(F)F)cc1Cl] |
| 77 | INT C27 | 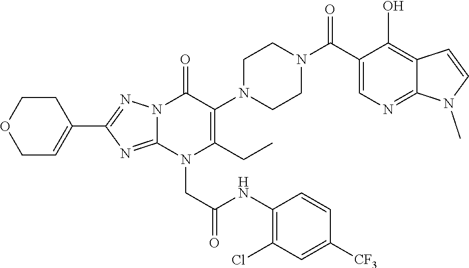 [SMILES: CCc1c(N2CCN(C(=O)c3cnc4c(ccn4C)c3O)CC2)c(=O)n2nc(C3=CCOCC3)nc2n1CC(=O)Nc1ccc(C(F)(F)F)cc1Cl] |
| 78 | 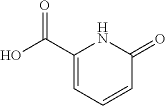 | 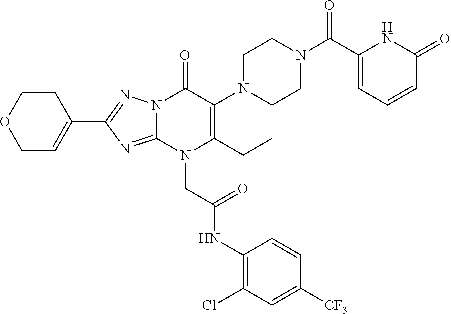 [SMILES: CCc1c(N2CCN(C(=O)c3cccc(=O)[nH]3)CC2)c(=O)n2nc(C3=CCOCC3)nc2n1CC(=O)Nc1ccc(C(F)(F)F)cc1Cl] |
| 79 | 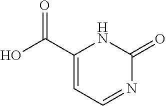 | 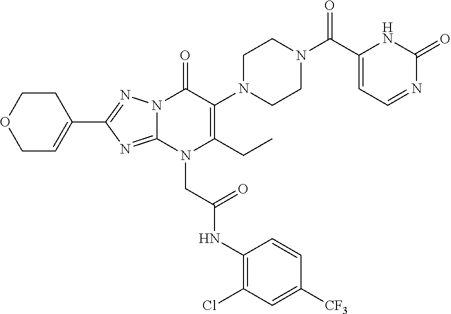 [SMILES: CCc1c(N2CCN(C(=O)c3ccnc(=O)[nH]3)CC2)c(=O)n2nc(C3=CCOCC3)nc2n1CC(=O)Nc1ccc(C(F)(F)F)cc1Cl] |
| 80 | INT C39 | 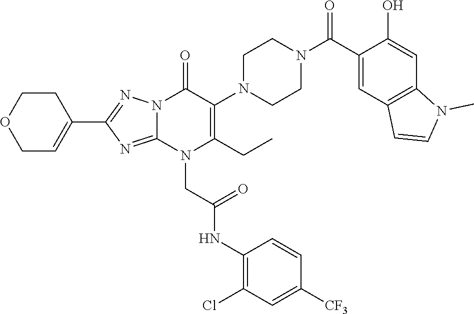 [SMILES: CCc1c(N2CCN(C(=O)c3cc4ccn(C)c4cc3O)CC2)c(=O)n2nc(C3=CCOCC3)nc2n1CC(=O)Nc1ccc(C(F)(F)F)cc1Cl] |
| 81 | INT C28 | 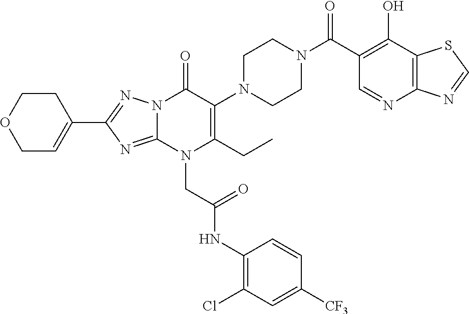 [SMILES: CCc1c(N2CCN(C(=O)c3cnc4ncsc4c3O)CC2)c(=O)n2nc(C3=CCOCC3)nc2n1CC(=O)Nc1ccc(C(F)(F)F)cc1Cl] |
| 82 | INT C29 | 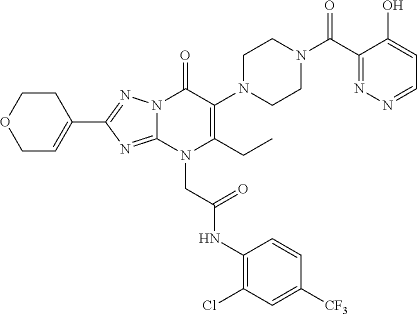 [SMILES: CCc1c(N2CCN(C(=O)c3nnccc3O)CC2)c(=O)n2nc(C3=CCOCC3)nc2n1CC(=O)Nc1ccc(C(F)(F)F)cc1Cl] |
| 83 | INT C30 | 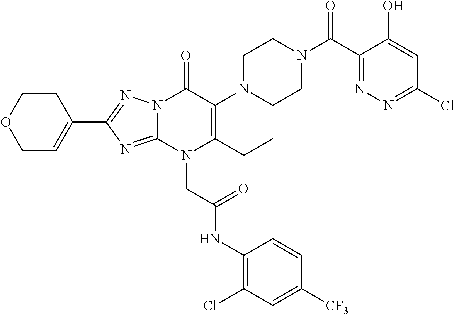 [SMILES: CCc1c(N2CCN(C(=O)c3nnc(Cl)cc3O)CC2)c(=O)n2nc(C3=CCOCC3)nc2n1CC(=O)Nc1ccc(C(F)(F)F)cc1Cl] |
| 84 | INT C31 | 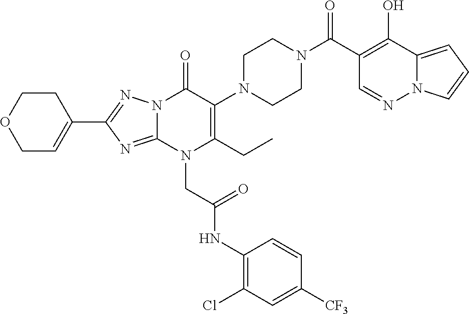 [SMILES: CCc1c(N2CCN(C(=O)c3cnn4cccc4c3O)CC2)c(=O)n2nc(C3=CCOCC3)nc2n1CC(=O)Nc1ccc(C(F)(F)F)cc1Cl] |
| 85 | INT C32 | 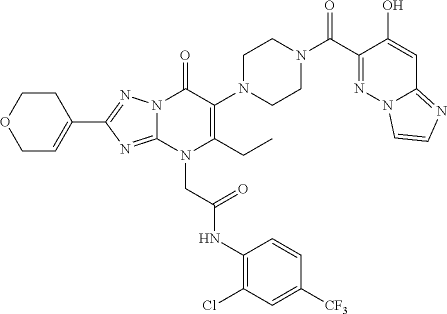 [SMILES: CCc1c(N2CCN(C(=O)c3nn4ccnc4cc3O)CC2)c(=O)n2nc(C3=CCOCC3)nc2n1CC(=O)Nc1ccc(C(F)(F)F)cc1Cl] |
| 86 | 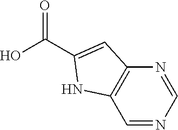 [SMILES: O=C(O)c1cc2ncncc2[nH]1] | 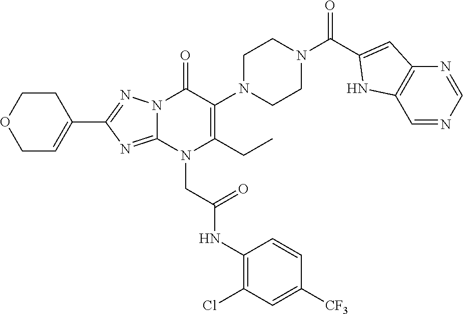 [SMILES: CCc1c(N2CCN(C(=O)c3cc4ncncc4[nH]3)CC2)c(=O)n2nc(C3=CCOCC3)nc2n1CC(=O)Nc1ccc(C(F)(F)F)cc1Cl] |
| 87 | INT C33 | 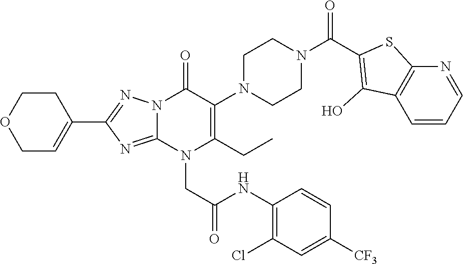 [SMILES: CCc1c(N2CCN(C(=O)c3sc4ncccc4c3O)CC2)c(=O)n2nc(C3=CCOCC3)nc2n1CC(=O)Nc1ccc(C(F)(F)F)cc1Cl] |
| 88 | INT C34 | 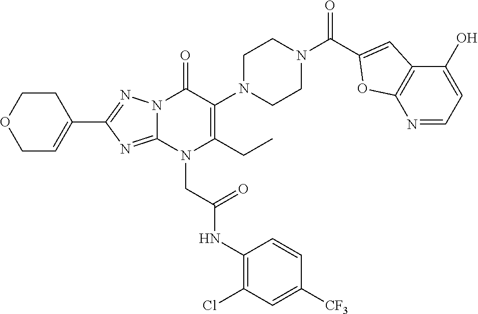 [SMILES: CCc1c(N2CCN(C(=O)c3cc4c(O)ccnc4o3)CC2)c(=O)n2nc(C3=CCOCC3)nc2n1CC(=O)Nc1ccc(C(F)(F)F)cc1Cl] |
| 89 | 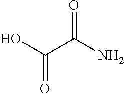 | 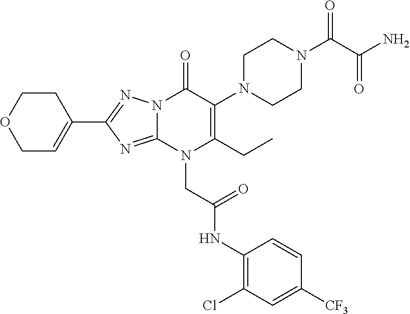 [SMILES: CCc1c(N2CCN(C(=O)C(N)=O)CC2)c(=O)n2nc(C3=CCOCC3)nc2n1CC(=O)Nc1ccc(C(F)(F)F)cc1Cl] |
| 90 | 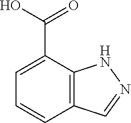 [SMILES: O=C(O)c1cccc2cn[nH]c12] | 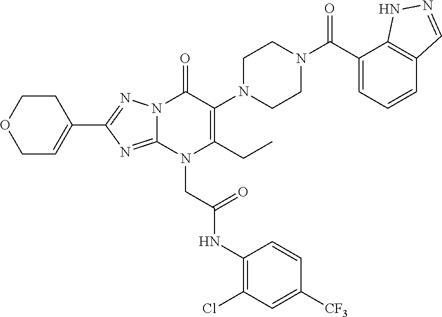 [SMILES: CCc1c(N2CCN(C(=O)c3cccc4cn[nH]c34)CC2)c(=O)n2nc(C3=CCOCC3)nc2n1CC(=O)Nc1ccc(C(F)(F)F)cc1Cl] |
| 91 | 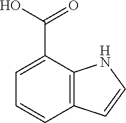 [SMILES: O=C(O)c1cccc2cc[nH]c12] | 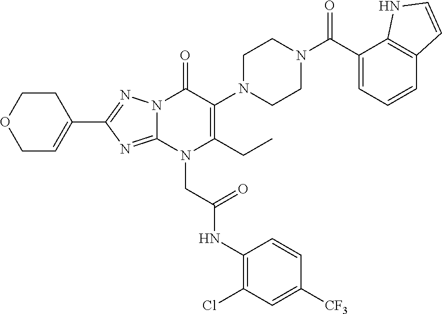 [SMILES: CCc1c(N2CCN(C(=O)c3cccc4cc[nH]c34)CC2)c(=O)n2nc(C3=CCOCC3)nc2n1CC(=O)Nc1ccc(C(F)(F)F)cc1Cl] |
| 92 | INT C43 | 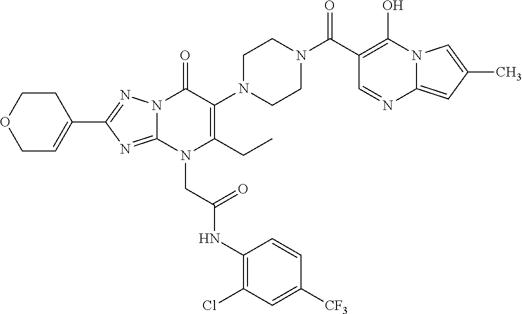 [SMILES: CCc1c(N2CCN(C(=O)c3cnc4cc(C)cn4c3O)CC2)c(=O)n2nc(C3=CCOCC3)nc2n1CC(=O)Nc1ccc(C(F)(F)F)cc1Cl] |

Preparations of Examples (Ex)

| | | LCMS |
|---|---|---|
| | | Cacl./ |
| Ex | Name | Found |
| 43 | N-(2-Chloro-4- | [M + H]⁺: |
| | (trifluoromethyl)phenyl)-2-(2-(3,6- | 718.2/ |
| | dihydro-2H-pyran-4-yl)-5-ethyl-6-(4- | 718.2. |
| | (3-methyl-2,4-dioxo-1,2,3,4- | |
| | tetrahydropyrimidine-5- | |
| | carbonyl)piperazin-1-yl)-7-oxo- | |
| | [1,2,4]triazolo[1,5-a]pyrimidin- | |
| | 4(7H)-yl)acetamide | |
| 44 | N-(2-Chloro-4- | [M + H]⁺: |
| | (trifluoromethyl)phenyl)-2-(6-(4-(7- | 745.1/ |
| | chloro-5H-pyrrolo[3,2-d]pyrimidine- | 745.0. |
| | 4-carbonyl)piperazin-1-yl)-2-(3,6- | |
| | dihydro-2H-pyran-4-yl)-5-ethyl-7- | |
| | oxo-[1,2,4]triazolo[1,5-a]pyrimidin- | |
| | 4(7H)-yl)acetamide | |
| 45 | N-(2-Chloro-4- | [M + H]⁺: |
| | (trifluoromethyl)phenyl)-2-(2-(3,6- | 743.2/ |
| | dihydro-2H-pyran-4-yl)-5-ethyl-6-(4- | 743.0. |
| | (7-hydroxythieno[3,2-b]pyridine-6- | |
| | carbonyl)piperazin-1-yl)-7-oxo- | |
| | [1,2,4]triazolo[1,5-a]pyrimidin- | |
| | 4(7H)-yl)acetamide | |
| 46 | 2-(6-(4-(3-Amino-5- | [M + H]⁺: |
| | fluoropicolinoyl)piperazin-1-yl)-2- | 704.2/ |
| | (3,6-dihydro-2H-pyran-4-yl)-5-ethyl- | 704.0. |
| | 7-oxo-[1,2,4]triazolo[1,5- | |
| | a]pyrimidin-4(7H)-yl)-N-(2-chloro-4- | |
| | (trifluoromethyl)phenyl)acetamide | |
| 47 | N-(2-Chloro-4- | [M + H]⁺: |
| | (trifluoromethyl)phenyl)-2-(2-(3,6- | 688.2/ |
| | dihydro-2H-pyran-4-yl)-5-ethyl-7- | 688.0 |
| | oxo-6-(4-(3-oxo-3,4- | |
| | dihydropyrazine-2- | |
| | carbonyl)piperazin-1-yl)- | |
| | [1,2,4]triazolo[1,5-a]pyrimidin- | |
| | 4(7H)-yl)acetamide | |
| 48 | N-(2-Chloro-4- | [M + H]⁺: |
| | (trifluoromethyl)phenyl)-2-(2-(3,6- | 704.2/ |
| | dihydro-2H-pyran-4-yl)-5-ethyl-6-(4- | 704.0 |
| | (4-hydroxy-6-oxo-1,6- | |
| | dihydropyridazine-3- | |
| | carbonyl)piperazin-1-yl)-7-oxo- | |
| | [1,2,4]triazolo[1,5-a]pyrimidin- | |
| | 4(7H)-yl)acetamide | |
| 49 | N-(2-Chloro-4- | [M + H]⁺: |
| | (trifluoromethyl)phenyl)-2-(2-(3,6- | 744.2/ |
| | dihydro-2H-pyran-4-yl)-5-ethyl-6-(4- | 744.0. |
| | (7-hydroxythiazolo[5,4-b]pyridine-6- | |
| | carbonyl)piperazin-1-yl)-7-oxo- | |
| | [1,2,4]triazolo[1,5-a]pyrimidin- | |
| | 4(7H)-yl)acetamide | |
| 50 | N-(2-Chloro-4- | [M + H]⁺: |
| | (trifluoromethyl)phenyl)-2-(2-(3,6- | 704.2/ |
| | dihydro-2H-pyran-4-yl)-6-(4-(2,4- | 704.4. |
| | dioxo-1,2,3,4-tetrahydropyrimidine- | |
| | 5-carbonyl)piperazin-1-yl)-5-ethyl-7- | |
| | oxo-[1,2,4]triazolo[1,5-a]pyrimidin- | |
| | 4(7H)-yl)acetamide | |
| 51 | N-(2-Chloro-4- | [M + H]⁺: |
| | (trifluoromethyl)phenyl)-2-(2-(3,6- | 732.2/ |
| | dihydro-2H-pyran-4-yl)-6-(4-(1,3- | 732.2. |
| | dimethyl-2,4-dioxo-1,2,3,4- | |
| | tetrahydropyrimidine-5- | |
| | carbonyl)piperazin-1-yl)-5-ethyl-7- | |
| | oxo-[1,2,4]triazolo[1,5-a]pyrimidin- | |
| | 4(7H)-yl)acetamide | |
| 52 | N-(2-Chloro-4- | [M + H]⁺: |
| | (trifluoromethyl)phenyl)-2-(2-(3,6- | 728.2/ |
| | dihydro-2H-pyran-4-yl)-5-ethyl-6-(4- | 728.0. |
| | (7-hydroxy-[1,2,4]triazolo[1,5- | |
| | a]pyrimidine-6-carbonyl)piperazin-1 - | |
| | yl)-7-oxo-[1,2,4]triazolo[1,5- | |
| | a]pyrimidin-4(7H)-yl)acetamide | |
| 53 | Ethyl 2-(4-(4-(2-((2-chloro-4- | [M + H]⁺: |
| | (trifluoromethyl)phenyl)amino)-2- | 799.2/ |
| | oxoethyl)-2-(3,6-dihydro-2H-pyran- | 799.2. |
| | 4-yl)-5-ethyl-7-oxo-4,7-dihydro- | |
| | [1,2,4]triazolo[1,5-a]pyrimidin-6- | |
| | yl)piperazine-1-carbonyl)-4- | |
| | hydroxyfuro[2,3-b]pyridine-5- | |
| | carboxylate | |
| 54 | N-(2-Chloro-4- | [M + H]⁺: |
| | (trifluoromethyl)phenyl)-2-(2-(3,6- | 702.2/ |
| | dihydro-2H-pyran-4-yl)-5-ethyl-6-(4- | 702.2. |
| | (4-methyl-3-oxo-3,4- | |
| | dihydropyrazine-2- | |
| | carbonyl)piperazin-1-yl)-7-oxo- | |
| | [1,2,4]triazolo[1,5-a]pyrimidin- | |
| | 4(7H)-yl)acetamide | |
| 55 | N-(2-Chloro-4- | [M + H]⁺: |
| | (trifluoromethyl)phenyl)-2-(2-(3,6- | 743.2/ |
| | dihydro-2H-pyran-4-yl)-5-ethyl-6-(4- | 743.2. |
| | (4-hydroxythieno[2,3-b]pyridine-5- | |
| | carbonyl)piperazin-1-yl)-7-oxo- | |
| | [1,2,4]triazolo[1,5-a]pyrimidin- | |
| | 4(7H)-yl)acetamide | |
| 56 | N-(2-Chloro-4- | [M + H]⁺: |
| | (trifluoromethyl)phenyl)-2-(2-(3,6- | 703.2/ |
| | dihydro-2H-pyran-4-yl)-5-ethyl-6-(4- | 702.8. |
| | (3-methyl-5-oxo-4,5-dihydro-1,2,4- | |
| | triazine-6-carbonyl)piperazin-1-yl)-7- | |
| | oxo-[1,2,4]triazolo[1,5-a]pyrimidin- | |
| | 4(7H)-yl)acetamide | |
| 57 | N-(2-Chloro-4- | [M + H]⁺: |
| | (trifluoromethyl)phenyl)-2-(2-(3,6- | 727.2/ |
| | dihydro-2H-pyran-4-yl)-5-ethyl-6-(4- | 727.2 |
| | (7-hydroxy-3H-imidazo[4,5- | |
| | b]pyridine-6-carbonyl)piperazin-1- | |
| | yl)-7-oxo-[1,2,4]triazolo[1,5- | |
| | a]pyrimidin-4(7H)-yl)acetamide | |
| 58 | N-(2-Chloro-4- | [M + H]⁺: |
| | (trifluoromethyl)phenyl)-2-(2-(3,6- | 719.2/ |
| | dihydro-2H-pyran-4-yl)-5-ethyl-6-(4- | 718.8 |
| | (2-methyl-3,5-dioxo-2,3,4,5- | |
| | tetrahydro-1,2,4-triazine-6- | |
| | carbonyl)piperazin-1-yl)-7-oxo- | |
| | [1,2,4]triazolo[1,5-a]pyrimidin- | |
| | 4(7H)-yl)acetamide | |
| 59 | N-(2-Chloro-4- | [M + H]⁺: |
| | (trifluoromethyl)phenyl)-2-(2-(3,6- | 718.2/ |
| | dihydro-2H-pyran-4-yl)-5-ethyl-6-(4- | 718.2 |
| | (1-methyl-2,4-dioxo-1,2,3,4- | |
| | tetrahydropyrimidine-5- | |
| | carbonyl)piperazin-1-y1)-7-oxo- | |
| | [1,2,4]triazolo[1,5-a]pyrimidin- | |
| | 4(7H)-yl)acetamide | |
| 60 | N-(2-Chloro-4- | [M + H]⁺: |
| | (trifluoromethyl)phenyl)-2-(2-(3,6- | 700.2/ |
| | dihydro-2H-pyran-4-yl)-5-ethyl-6-(4- | 699.8 |
| | (2-hydroxy-3- | |
| | methylbenzoyl)piperazin-1-yl)-7- | |
| | oxo-[1,2,4]triazolo[1,5-a]pyrimidin- | |
| | 4(7H)-yl)acetamide | |
| 61 | 2-(6-(4-(1H-Pyrrolo[2,3-c]pyridine- | [M + H]⁺: |
| | 7-carbonyl)piperazin-1-yl)-2-(3,6- | 710.2/ |
| | dihydro-2H-pyran-4-yl)-5-ethyl-7- | 709.8 |
| | oxo-[1,2,4]triazolo[1,5-a]pyrimidin- | |
| | 4(7H)-yl)-N-(2-chloro-4- | |
| | (trifluoromethyl)phenyl)acetamide | |
| 62 | 2-(6-(4-(3H-Imidazo[4,5-c]pyridine- | [M + H]⁺: |
| | 4-carbonyl)piperazin-1-yl)-2-(3,6- | 711.2/ |
| | dihydro-2H-pyran-4-yl)-5-ethyl-7- | 710.8 |
| | oxo-[1,2,4]triazolo[1,5-a]pyrimidin- | |
| | 4(7H)-yl)-N-(2-chloro-4- | |
| | (trifluoromethyl)phenyl)acetamide | |
| 63 | N-(2-Chloro-4- | [M + H]⁺: |
| | (trifluoromethyl)phenyl)-2-(2-(3,6- | 676.2/ |
| | dihydro-2H-pyran-4-yl)-5-ethyl-7- | 675.8 |
| | oxo-6-(4-(3-oxo-2,3-dihydro-1H- | |
| | pyrazole-4-carbonyl)piperazin-1-yl)- | |
| | [1,2,4]triazolo[1,5-a]pyrimidin- | |
| | 4(7H)-yl)acetamide | |
| 64 | N-(2-Chloro-4- | [M + H]⁺: |
| | (trifluoromethyl)phenyl)-2-(2-(3,6- | 690.2/ |
| | dihydro-2H-pyran-4-yl)-5-ethyl-6-(4- | 689.8 |
| | (1-methyl-3-oxo-2,3-dihydro-1H- | |
| | pyrazole-4-carbonyl)piperazin-1-yl)- | |
| | 7-oxo-[1,2,4]triazolo[1,5- | |
| | a]pyrimidin-4(7H)-yl)acetamide | |
| 65 | N-(2-Chloro-4- | [M + H]⁺: |
| | (trifluoromethyl)phenyl)-2-(2-(3,6- | 687.2/ |
| | dihydro-2H-pyran-4-yl)-5-ethyl-7- | 686.8 |
| | oxo-6-(4-(2-oxo-1,2-dihydropyridine- | |
| | 3-carbonyl)piperazin-1-yl)- | |
| | [1,2,4]triazolo[1,5-a]pyrimidin- | |
| | 4(7H)-yl)acetamide | |
| 66 | N-(2-Chloro-4- | [M + H]⁺: |
| | (trifluoromethyl)phenyl)-2-(2-(3,6- | 677.2/ |
| | dihydro-2H-pyran-4-yl)-5-ethyl-7- | 676.9 |
| | oxo-6-(4-(5-oxopyrrolidine-2- | |
| | carbonyl)piperazin-1-yl)- | |
| | [1,2,4]triazolo[1,5-a]pyrimidin- | |
| | 4(7H)-yl)acetamide | |
| 67 | 2-(6-(4-(1H-Pyrrolo[3,2-c]pyridine- | [M + H]⁺: |
| | 4-carbonyl)piperazin-1-yl)-2-(3,6- | 710.2/ |
| | dihydro-2H-pyran-4-yl)-5-ethyl-7- | 710.4 |
| | oxo-[1,2,4]triazolo[1,5-a]pyrimidin- | |
| | 4(7H)-yl)-N-(4-chloro-2- | |
| | (trifluoromethyl)phenyl)acetamide | |
| 68 | 2-(6-(4-(1H-Pyrrolo[3,2-b]pyridine- | [M + H]⁺: |
| | 7-carbonyl)piperazin-1-yl)-2-(3,6- | 710.2/ |
| | dihydro-2H-pyran-4-yl)-5-ethyl-7- | 710.3 |
| | oxo-[1,2,4]triazolo[1,5-a]pyrimidin- | |
| | 4(7H)-yl)-N-(4-chloro-2- | |
| | (trifluoromethyl)phenyl)acetamide | |
| 69 | 2-(6-(4-(7H-Pyrrolo[2,3- | [M + H]⁺: |
| | d]pyrimidine-4-carbonyl)piperazin-1- | 711.2/ |
| | yl)-2-(3,6-dihydro-2H-pyran-4-yl)-5- | 711.3 |
| | ethyl-7-oxo-[1,2,4]triazolo[1,5- | |
| | a]pyrimidin-4(7H)-yl)-N-(4-chloro-2- | |
| | (trifluoromethyl)phenyl)acetamide | |
| 70 | N-(4-Chloro-2- | [M + H]⁺: |
| | (trifluoromethyl)phenyl)-2-(2-(3,6- | 728.2/ |
| | dihydro-2H-pyran-4-yl)-5-ethyl-7- | 728.4 |
| | oxo-6-(4-(2-(trifluoromethyl)-1H- | |
| | imidazole-5-carbonyl)piperazin-1- | |
| | yl)-[1,2,4]triazolo[1,5-a]pyrimidin- | |
| | 4(7H)-yl)acetamide | |
| 71 | N-(2-Chloro-4- | [M + H]⁺: |
| | (trifluoromethyl)phenyl)-2-(2-(3,6- | 743.2/ |
| | dihydro-2H-pyran-4-yl)-5-ethyl-7- | 743.2 |
| | oxo-6-(4-(6-oxo-6,7- | |
| | dihydrothieno[2,3-b]pyridine-5- | |
| | carbonyl)piperazin-1-yl)- | |
| | [1,2,4]triazolo[1,5-a]pyrimidin- | |
| | 4(7H)-yl)acetamide | |
| 72 | N-(2-Chloro-4- | [M + H]⁺: |
| | (trifluoromethyl)phenyl)-2-(2-(3,6- | 740.2/ |
| | dihydro-2H-pyran-4-yl)-5-ethyl-6-(4- | 740.0 |
| | (4-hydroxy-2-methylpyrazolo[1,5- | |
| | a]pyridine-5-carbonyl)piperazin-1- | |
| | yl)-7-oxo-[1,2,4]triazolo[1,5- | |
| | a]pyrimidin-4(7H)-yl)acetamide | |
| 73 | N-(4-Chloro-2- | [M + H]⁺: |
| | (trifluoromethyl)phenyl)-2-(2-(3,6- | 719.2/ |
| | dihydro-2H-pyran-4-yl)-5-ethyl-6-(4- | 719.0 |
| | (4-methyl-3,5-dioxo-2,3,4,5- | |
| | tetrahydro-1,2,4-triazine-6- | |
| | carbonyl)piperazin-1-yl)-7-oxo- | |
| | [1,2,4]triazolo[1,5-a]pyrimidin- | |
| | 4(7H)-yl)acetamide | |
| 74 | N-(2-Chloro-4- | [M + H]⁺: |
| | (trifluoromethyl)phenyl)-2-(2-(3,6- | 743.2/ |
| | dihydro-2H-pyran-4-yl)-5-ethyl-6-(4- | 743.4 |
| | (3-hydroxythieno[2,3-c]pyridine-2- | |
| | carbonyl)piperazin-1-y1)-7-oxo- | |
| | [1,2,4]triazolo[1,5-a]pyrimidin- | |
| | 4(7H)-yl)acetamide | |
| 75 | N-(2-Chloro-4- | [M + H]⁺: |
| | (trifluoromethyl)phenyl)-2-(2-(3,6- | 727.2/ |
| | dihydro-2H-pyran-4-yl)-5-ethyl-6-(4- | 727.2 |
| | (3-hydroxyimidazo[1,2-a]pyrazine-2- | |
| | carbonyl)piperazin-1-yl)-7-oxo- | |
| | [1,2,4]triazolo[1,5-a]pyrimidin- | |
| | 4(7H)-yl)acetamide | |
| 76 | N-(2-Chloro-4- | [M + H]⁺: |
| | (trifluoromethyl)phenyl)-2-(2-(3,6- | 702.2./ |
| | dihydro-2H-pyran-4-yl)-5-ethyl-6-(4- | 702.2 |
| | (5-methyl-4-oxo-1,4- | |
| | dihydropyridazine-3- | |
| | carbonyl)piperazin-1-yl)-7-oxo- | |
| | [1,2,4]triazolo[1,5-a]pyrimidin- | |
| | 4(7H)-yl)acetamide | |
| 77 | N-(2-Chloro-4- | [M + H]⁺: |
| | (trifluoromethyl)phenyl)-2-(2-(3,6- | 740.2/ |
| | dihydro-2H-pyran-4-yl)-5-ethyl-6-(4- | 740.0 |
| | (4-hydroxy-1-methyl-1H-pyrrolo[2,3- | |
| | b]pyridine-5-carbonyl)piperazin-1- | |
| | yl)-7-oxo-[1,2,4]triazolo[1,5- | |
| | a]pyrimidin-4(7H)-yl)acetamide | |
| 78 | N-(2-Chloro-4- | [M + H]⁺: |
| | (trifluoromethyl)phenyl)-2-(2-(3,6- | 687.2/ |
| | dihydro-2H-pyran-4-yl)-5-ethyl-7- | 687.0 |
| | oxo-6-(4-(6-oxo-1,6-dihydropyridine- | |
| | 2-carbonyl)piperazin-1-yl)- | |
| | [1,2,4]triazolo[1,5-a]pyrimidin- | |
| | 4(7H)-yl)acetamide | |
| 79 | N-(2-Chloro-4- | [M + H]⁺: |
| | (trifluoromethyl)phenyl)-2-(2-(3,6- | 688.2/ |
| | dihydro-2H-pyran-4-yl)-5-ethyl-7- | 688.0 |
| | oxo-6-(4-(2-oxo-2,3- | |
| | dihydropyrimidine-4- | |
| | carbonyl)piperazin-1-yl)- | |
| | [1,2,4]triazolo[1,5-a]pyrimidin- | |
| | 4(7H)-yl)acetamide | |
| 80 | N-(2-Chloro-4- | [M + H]⁺: |
| | (trifluoromethyl)phenyl)-2-(2-(3,6- | 739.2/ |
| | dihydro-2H-pyran-4-yl)-5-ethyl-6-(4- | 739.2 |
| | (6-hydroxy-1-methyl-1H-indole-5- | |
| | carbonyl)piperazin-1-yl)-7-oxo- | |
| | [1,2,4]triazolo[1,5-a]pyrimidin- | |
| | 4(7H)-yl)acetamide | |
| 81 | N-(2-Chloro-4- | [M + H]⁺: |
| | (trifluoromethyl)phenyl)-2-(2-(3,6- | 744.2/ |
| | dihydro-2H-pyran-4-yl)-5-ethyl-6-(4- | 744.1 |
| | (7-hydroxythiazolo[4,5-b]pyridine-6- | |
| | carbonyl)piperazin-1-yl)-7-oxo- | |
| | [1,2,4]triazolo[1,5-a]pyrimidin- | |
| | 4(7H)-yl)acetamide | |
| 82 | N-(2-Chloro-4- | [M + H]⁺: |
| | (trifluoromethyl)phenyl)-2-(2-(3,6- | 688.2/ |
| | dihydro-2H-pyran-4-yl)-5-ethyl-6-(4- | 688.0 |
| | (4-hydroxypyridazine-3- | |
| | carbonyl)piperazin-1-yl)-7-oxo- | |
| | [1,2,4]triazolo[1,5-a]pyrimidin- | |
| | 4(7H)-yl)acetamide | |
| 83 | N-(2-Chloro-4- | [M + H]⁺: |
| | (trifluoromethyl)phenyl)-2-(6-(4-(6- | 722.1/ |
| | chloro-4-hydroxypyridazine-3- | 722.0 |
| | carbonyl)piperazin-1-yl)-2-(3,6- | |
| | dihydro-2H-pyran-4-yl)-5-ethyl-7- | |
| | oxo-[1,2,4]triazolo[1,5-a]pyrimidin- | |
| | 4(7H)-yl)acetamide | |
| 84 | N-(2-Chloro-4- | [M + H]⁺: |
| | (trifluoromethyl)phenyl)-2-(2-(3,6- | 726.2/ |
| | dihydro-2H-pyran-4-yl)-5-ethyl-6-(4- | 726.0 |
| | (4-hydroxypyrrolo[1,2-b]pyridazine- | |
| | 3-carbonyl)piperazin-1-yl)-7-oxo- | |
| | [1,2,4]triazolo[1,5-a]pyrimidin- | |
| | 4(7H)-yl)acetamide | |
| 85 | N-(2-Chloro-4- | [M + H]⁺: |
| | (trifluoromethyl)phenyl)-2-(2-(3,6- | 727.2/ |
| | dihydro-2H-pyran-4-yl)-5-ethyl-6-(4- | 727.1 |
| | (7-hydroxyimidazo[1,2-b]pyridazine- | |
| | 6-carbonyl)piperazin-1-yl)-7-oxo- | |
| | [1,2,4]triazolo[1,5-a]pyrimidin- | |
| | 4(7H)-yl)acetamide | |
| 86 | 2-(6-(4-(5H-Pyrrolo[3,2- | [M + H]⁺: |
| | d]pyrimidine-6-carbonyl)piperazin-1- | 711.2/ |
| | yl)-2-(3,6-dihydro-2H-pyran-4-yl)-5- | 711.0 |
| | ethyl-7-oxo-[1,2,4]triazolo[1,5- | |
| | a]pyrimidin-4(7H)-yl)-N-(2-chloro-4- | |
| | (trifluoromethyl)phenyl)acetamide | |
| 87 | N-(2-Chloro-4- | [M + H]⁺: |
| | (trifluoromethyl)phenyl)-2-(2-(3,6- | 743.2/ |
| | dihydro-2H-pyran-4-yl)-5-ethyl-6-(4- | 743.3 |
| | (3-hydroxythieno[2,3-b]pyridine-2- | |
| | carbonyl)piperazin-1-yl)-7-oxo- | |
| | [1,2,4]triazolo[1,5-a]pyrimidin- | |
| | 4(7H)-yl)acetamide | |
| 88 | N-(2-Chloro-4- | [M + H]⁺: |
| | (trifluoromethyl)phenyl)-2-(2-(3,6- | 727.2/ |
| | dihydro-2H-pyran-4-yl)-5-ethyl-6-(4- | 727.3 |
| | (4-hydroxyfuro[2,3-b]pyridine-2- | |
| | carbonyl)piperazin-1-yl)-7-oxo- | |
| | [1,2,4]triazolo[1,5-a]pyrimidin- | |
| | 4(7H)-yl)acetamide | |
| 89 | 2-(6-(4-(2-Amino-2- | [M + H]⁺: |
| | oxoacetyl)piperazin-1-yl)-2-(3,6- | 637.2/ |
| | dihydro-2H-pyran-4-yl)-5-ethyl-7- | 637.0 |
| | oxo-[1,2,4]triazolo[1,5-a]pyrimidin- | |
| | 4(7H)-yl)-N-(2-chloro-4- | |
| | (trifluoromethyl)phenyl)acetamide | |
| 90 | 2-(6-(4-(1H-Indazole-7- | [M + H]⁺: |
| | carbonyl)piperazin-1-yl)-2-(3,6- | 710.2/ |
| | dihydro-2H-pyran-4-yl)-5-ethyl-7- | 710.2 |
| | oxo-[1,2,4]triazolo[1,5-a]pyrimidin- | |
| | 4(7H)-yl)-N-(2-chloro-4- | |
| | (trifluoromethyl)phenyl)acetamide | |
| 91 | 2-(6-(4-(1H-Indole-7- | [M + H]⁺: |
| | carbonyl)piperazin-1-yl)-2-(3,6- | 709.2/ |
| | dihydro-2H-pyran-4-y1)-5-ethyl-7- | 709.0 |
| | oxo-[1,2,4]triazolo[1,5-a]pyrimidin- | |
| | 4(7H)-yl)-N-(2-chloro-4- | |
| | (trifluoromethyl)phenyl)acetamide | |
| 92 | N-(2-Chloro-4- | [M + H]⁺: |
| | (trifluoromethyl)phenyl)-2-(2-(3,6- | 740.2/ |
| | dihydro-2H-pyran-4-yl)-5-ethyl-6-(4- | 740.5 |
| | (4-hydroxy-7-methylpyrrolo[1,2- | |
| | a]pyrimidine-3-carbonyl)piperazin-1 - | |
| | yl)-7-oxo-[1,2,4]triazolo[1,5- | |
| | a]pyrimidin-4(7H)-yl)acetamide | |

TABLE 2
¹H NMR of Examples (Ex)

| Ex | ¹H NMR (400 MHz, DMSO-d₆) δ ppm |
|---|---|
| 43 | 10.78 (d, J = 297.2 Hz, 2H), 8.06 (dd, J = 8.6, 0.9 Hz, 1H), 7.97-7.96 (m, 1H), 7.72 (d, J = 5.0 Hz, 2H), |
| | 6.83 (t, J = 1.5 Hz, 1H), 5.32 (s, 2H), 4.44 (s, 1H), 4.25 (q, J = 2.8 Hz, 2H), 3.80 (t, J = 5.4 Hz, 2H), 3.60 |
| | (s, 1H), 3.47-3.40 (m, 2H), 3.29 (s, 2H), 3.14 (s, 3H), 2.98 (d, J = 7.6 Hz, 2H), 2.94-2.54 (m, 4H), |
| | 1.19 (t, J = 7.5 Hz, 3H). |
| 44 | 12.48 (s, 1H), 10.37 (s, 1H), 8.95 (s, 1H), 8.16 (s, 1H), 8.05 (d, J = 8.6 Hz, 1H), 7.96 (s, 1H), 7.72 (d, J = |
| | 7.2 Hz, 1H), 6.83 (s, 1H), 5.32 (s, 2H), 4.63 (d, J = 12.2 Hz, 1H), 4.28-4.22 (m, 2H), 3.91 (d, J = 12.2 |
| | Hz, 1H), 3.80 (t, J = 5.4 Hz, 2H), 3.53 (dt, J = 36.6, 10.8 Hz, 4H), 3.16-2.94 (m, 4H), 2.88 (d, J = 10.8 |
| | Hz, 1H), 2.66 (d, J = 10.2 Hz, 1H), 1.20 (t, J = 7.4 Hz, 3H). |
| 45 | 10.38 (s, 1H), 8.09 (s, 1H), 8.06 (d, J = 8.6 Hz, 1H), 8.01 (d, J = 5.4 Hz, 1H), 7.96 (d, J = 2.0 Hz, 1H), |
| | 7.71 (dd, J = 8.7, 2.1 Hz, 1H), 7.27 (d, J = 5.3 Hz, 1H), 6.83 (s, 1H), 5.31 (s,2H), 4.53 (d, J = 12.5 Hz, |
| | 2H), 4.25 (q, J = 2.8 Hz, 2H), 3.80 (t, J = 5.5 Hz, 2H), 3.58-3.50 (m, 2H), 3.06-2.85 (m, 4H), 2.77 (d, |
| | J = 11.0 Hz, 2H), 2.69-2.58 (m, 2H), 1.19 (t, J = 7.4 Hz, 3H). |
| 46 | 10.36 (s, 1H), 8.06 (d, J = 8.5 Hz, 1H), 7.96 (d, J = 2.0 Hz, 1H), 7.75 (d, J = 2.5 Hz, 1H), 7.71 (dd, J = |
| | 8.9, 2.1 Hz, 1H), 6.96 (dd, J = 11.4, 2.5 Hz, 1H), 6.83 (t, J = 1.5 Hz, 1H), 5.91 (s, 2H), 5.31 (s, 2H), 4.54 |
| | (d, J = 12.6 Hz, 1H), 4.30-4.18 (m, 2H), 3.80 (t, J = 5.4 Hz, 3H), 3.47 (d, J = 11.5 Hz, 2H), 3.25 (s, |
| | 3H), 2.99 (d, J = 8.6 Hz, 3H), 2.80 (d, J = 11.4 Hz, 1H), 2.65 (t, J = 7.9 Hz, 1H), 1.19 (t, J = 7.4 Hz, 3H). |
| 47 | 8.69 (s, 2H), 8.05 (d, J = 8.5 Hz, 1H), 7.96 (d, J = 2.0 Hz, 1H), 7.71 (d, J = 8.7 Hz, 1H), 7.54 (s, 1H), |
| | 7.34 (s, 1H), 6.83 (s, 1H), 5.31 (s, 2H), 4.46 (d, J = 14.3 Hz, 1H), 4.25 (s, 2H), 3.80 (t, J = 5.3 Hz, 2H), |
| | 3.50-3.39 (m, 3H), 3.27 (s, 2H), 3.23 (s, 1H), 3.04-2.88 (m, 3H), 2.78 (d, J = 14.7 Hz, 1H), 2.63 |
| | (d, J = 10.5 Hz, 1H), 1.18 (t, J = 7.4 Hz, 3H). |
| 48 | 12.47 (s, 1H), 10.36 (s, 1H), 8.05 (d, J = 8.5 Hz, 1H), 7.96 (d, J = 2.1 Hz, 1H), 7.71 (dd, J = 8.8, 2.1 Hz, |
| | 1H), 6.83 (t, J = 1.6 Hz, 1H), 5.85 (s, 1H), 5.32 (d, J = 4.0 Hz, 2H), 4.45 (d, J = 12.4 Hz, 1H), 4.25 (q, J = |
| | 2.9 Hz, 2H), 3.80 (t, J = 5.5 Hz, 2H), 3.53-3.38 (m, 4H), 3.28-3.20 (m, 2H), 2.97 (tt, J = 14.0, 8.8 |
| | Hz, 3H), 2.78 (d, J = 11.1 Hz, 1H), 2.69-2.64 (m, 1H), 2.00 (q, J = 7.0, |
| | 6.6 Hz, 1H), 1.18 (t, J = 7.5 Hz, 3H). |
| 49 | 10.36 (s, 1H), 9.24 (s, 1H), 8.26 (s, 1H), 8.05 (d, J = 8.6 Hz, 1H), 7.98-7.94 (m, 1H), 7.71 (dd, J = 9.1, |
| | 2.1 Hz, 1H), 6.86-6.80 (m, 1H), 5.31 (s, 2H), 4.56 (s, 1H), 4.25 (d, J = 3.0 Hz, 2H), 3.80 (t, J = 5.5 Hz, |
| | 2H), 3.46 (s, 3H), 2.99 (d, J = 7.5 Hz, 2H), 2.84-2.60 (m, 3H), 1.19 (t, J = 7.5 Hz, 3H). |
| 50 | 11.07 (s, 1H), 10.52 (s, 1H), 8.06 (d, J = 8.5 Hz, 1H), 7.97 (d, J = 1.5 Hz, 1H), 7.72 (dd, J = 8.7, 1.6 Hz, |
| | 1H), 7.68 (s, 1H), 6.83 (s, 1H), 5.32 (s, 2H), 4.43 (s, 1H), 4.29-4.21 (m, 2H), 3.80 (t, J = 5.4 Hz, 2H), |
| | 3.58 (s, 1H), 3.47-3.39 (m, 3H), 3.20 (s, 2H), 2.98 (d, J = 7.4 Hz, 2H), 2.87 (s, 1H), 2.67 (s, 2H), 1.19 |
| | (t, J = 7.5 Hz, 3H). |
| 51 | 9.33 (s, 1H), 8.06 (d, J = 10.7 Hz, 2H), 7.95 (s, 1H), 7.71 (dd, J = 8.8, 2.1 Hz, 1H), 6.85-6.80 (m, 1H), |
| | 5.31 (s, 2H), 4.46 (d, J = 12.8 Hz, 1H), 4.31-4.20 (m, 2H), 3.80 (t, J = 5.4 Hz, 2H), 3.61 (d, J = 13.1 |
| | Hz, 1H), 3.43 (t, J = 10.8 Hz, 2H), 3.35 (s, 3H), 3.20 (s, 1H), 3.18 (s, 3H), 2.99 (q, J = 7.4 Hz, 2H), 2.93- |
| | 2.84 (m, 1H), 2.75 (d, J = 10.8 Hz, 1H), 2.63 (d, J = 10.3 Hz, 1H), 1.19 (t, J = 7.5 Hz, 3H). |
| 52 | 8.87 (d, J = 3.5 Hz, 1H), 8.71 (d, J = 8.4 Hz, 1H), 8.42 (s, 1H), 8.07 (d, J = 8.6 Hz, 1H), 7.97 (s, 1H), |
| | 7.72 (d, J = 7.7 Hz, 1H), 7.66 (dd, J = 8.4, 4.5 Hz, 1H), 6.85 (s, 1H), 5.34 (s, 2H), 4.36 (d, J = 12.1 Hz, |
| | 1H), 4.27 (s, 2H), 3.96 (d, J = 12.9 Hz, 1H), 3.82 (t, J = 5.4 Hz, 2H), 3.68 (s, 2H), 3.03 (d, J = 7.6 Hz, |
| | 3H), 2.94-2.79 (m, 3H), 2.08-1.92 (m, 2H), 1.22 (d, J = 7.7 Hz, 3H). |
| 53 | 11.99 (s, 1H), 10.37 (s, 1H), 8.69 (s, 1H), 8.07 (d, J = 8.3 Hz, 1H), 7.97 (s, 1H), 7.72 (d, J = 6.8 Hz, 1H), |
| | 7.57 (s, 1H), 6.83 (s, 1H), 5.33 (s, 2H), 4.37 (dd, J = 14.2, 7.1 Hz, 4H), 4.26 (s, 2H), 3.80 (t, J = 6.4 Hz, |
| | 2H), 3.50 (d, J = 9.5 Hz, 2H), 3.30-3.25 (m, 2H), 3.02 (d, J = 7.8 Hz, 3H), 2.87-2.66 (m, 3H), 1.35 (t, |
| | J = 7.1 Hz, 3H), 1.22 (t, J = 7.5 Hz, 3H). |
| 54 | 10.33 (s, 1H), 8.05 (d, J = 8.6 Hz, 1H), 7.96 (d, J = 2.0 Hz, 1H), 7.78 (d, J = 4.3 Hz, 1H), 7.71 (dd, J = |
| | 9.0, 2.1 Hz, 1H), 7.36 (d, J = 4.2 Hz, 1H), 6.83 (p, J = 1.5 Hz, 1H), 5.31 (s, 2H), 4.46 (d, J = 12.5 Hz, |
| | 1H), 4.25 (q, J = 2.8 Hz, 2H), 3.80 (t, J = 5.4 Hz, 2H), 3.49 (s, 3H), 3.47-3.38 (m, 3H), 3.30 (s, 1H), |
| | 3.23 (t, J = 11.7 Hz, 2H), 2.97 (dt, J = 13.6, 7.7 Hz, 3H), 2.79 (d, J = 11.3 Hz, 1H), 2.63 (d, J = 11.0 Hz, |
| | 1H), 1.18 (t, J = 7.4 Hz, 3H). |
| 55 | 10.35 (s, 1H), 8.09-8.04 (m, 2H), 7.97 (d, J = 8.1 Hz, 2H), 7.71 (d, J = 7.1 Hz, 1H), 7.26 (d, J = 5.5 Hz, |
| | 1H), 6.83 (s, 1H), 6.54 (s, 1H), 5.31 (s, 2H), 4.53 (d, J = 10.6 Hz, 1H), 4.25 (s, 2H), 3.80 (t, J = 5.4 Hz, |
| | 2H), 3.51 (d, J = 41.5 Hz, 4H), 2.96 (d, J = 36.8 Hz, 4H), 2.78 (s, 2H), 1.20 (t, J = 7.4 Hz, 4H). |
| 56 | 10.36 (s, 1H), 8.06 (d, J = 8.6 Hz, 1H), 7.96 (d, J = 2.1 Hz, 1H), 7.72 (dd, J = 8.7, 2.1 Hz, 1H), 6.83 (dd, |
| | J = 3.3, 1.7 Hz, 1H), 5.32 (s, 2H), 4.44 (d, J = 12.4 Hz, 1H), 4.25 (q, J = 2.9 Hz, 2H), 3.80 (t, J = 5.5 Hz, |
| | 2H), 3.54-3.39 (m, 4H), 3.23 (t, J = 12.0 Hz, 2H), 3.09-2.89 (m, 4H), 2.79 (d, J = 11.2 Hz, 1H), |
| | 2.67-2.61 (m, 1H), 2.29 (s, 3H), 1.18 (t, J = 7.5 Hz, 3H). |
| 57 | 10.36 (s, 1H), 8.11-8.02 (m, 2H), 7.96 (s, 1H), 7.79 (s, 1H), 7.71 (d, J = 8.0 Hz, 1H), 7.20 (s, 1H), 6.83 |
| | (s, 1H), 6.59 (d, J = 49.8 Hz, 1H), 5.31 (s, 3H), 4.51 (s, 1H), 4.25 (s, 2H), 3.80 (s, 2H), 2.98 (s, 4H), 2.75 |
| | (s, 3H), 2.04-1.95 (m, 3H), 1.12 (s, 3H). |
| 58 | 12.31 (s, 1H), 10.36 (s, 1H), 8.06 (d, J = 8.5 Hz, 1H), 7.97 (s, 1H), 7.72 (d, J = 8.5 Hz, 1H), 6.83 (s, 1H), |
| | 5.32 (s, 2H), 4.41 (d, J = 11.6 Hz, 1H), 4.25 (s, 2H), 3.90-3.74 (m, 3H), 3.42 (s, 6H), 3.21 (t, J = 11.4 |
| | Hz, 2H), 2.97 (d, J = 13.4 Hz, 3H), 2.79 (d, J = 10.3 Hz, 1H), 2.65 (d, J = 10.9 Hz, 1H), 1.19 (t, J = 7.2 |
| | Hz, 3H). |
| 59 | 10.37 (s, 1H), 8.06 (d, J = 8.6 Hz, 1H), 8.02 (s, 1H), 7.97 (d, J = 2.1 Hz, 1H), 7.72 (dd, J = 9.1, 2.1 Hz, |
| | 1H), 6.83 (t, J = 1.6 Hz, 1H), 5.32 (s, 2H), 4.44 (d, J = 12.4 Hz, 1H), 4.25 (q, J = 2.9 Hz, 2H), 3.80 (t, J = |
| | 5.5 Hz, 2H), 3.61 (d, J = 12.9 Hz, 1H), 3.45 (s, 2H), 3.28 (s, 3H), 3.28-3.08 (m, 2H), 2.99 (d, J = 7.5 |
| | Hz, 2H), 2.95-2.56 (m, 4H), 1.20 (d, J = 7.3 Hz, 3H). |
| 60 | 10.41 (s, 1H), 9.04 (s, 1H), 8.07-8.02 (m, 1H), 7.98-7.94 (m, 1H), 7.74-7.69 (m, 1H), 7.15 (ddd, J = |
| | 7.4, 1.8, 0.9 Hz, 1H), 7.03-6.99 (m, 1H), 6.85-6.79 (m, 2H), 5.32 (s, 2H), 4.25 (d, J = 2.9 Hz, 2H), |
| | 4.16-3.85 (m, 1H), 3.80 (t, J = 5.4 Hz, 2H), 3.50-3.41 (m, 2H), 3.30 (s, 1H), 3.09 (s, 2H), 2.98 (q, J = |
| | 7.4 Hz, 2H), 2.71 (d, J = 11.1 Hz, 2H), 2.20 (s, 3H), 1.19 (t, J = 7.4 Hz, 3H). |
| 61 | 11.63 (s, 1H), 10.37 (s, 1H), 8.13 (d, J = 5.4 Hz, 1H), 8.06 (d, J = 8.5 Hz, 1H), 7.97 (d, J = 2.1 Hz, 1H), |
| | 7.72 (dd, J = 8.8, 2.1 Hz, 1H), 7.65 (d, J = 5.4 Hz, 1H), 7.59 (t, J = 2.8 Hz, 1H), 6.83 (td, J = 3.0, 1.5 Hz, |
| | 1H), 6.58 (dd, J = 3.0, 1.7 Hz, 1H), 5.32 (s, 2H), 4.68 (d, J = 12.4 Hz, 1H), 4.29-4.19 (m, 3H), 3.80 (t, J = |
| | 5.4 Hz, 2H), 3.62-3.48 (m, 2H), 3.16-3.05 (m, 1H), 3.01 (q, J = 7.8 Hz, 2H), 2.86 (d, J = 11.2 Hz, |
| | 1H), 2.70-2.64 (m, 1H), 1.20 (t, J = 7.5 Hz, 3H). |
| 62 | 10.37 (s, 1H), 8.44 (s, 1H), 8.33 (d, J = 5.5 Hz, 1H), 8.05 (d, J = 8.5 Hz, 1H), 7.96 (d, J = 2.1 Hz, 1H), |
| | 7.71 (dd, J = 8.9, 2.2 Hz, 2H), 6.87-6.80 (m, 1H), 5.32 (s, 2H), 4.66 (d, J = 12.4 Hz, 1H), 4.25 (q, J = |
| | 2.8 Hz, 2H), 3.80 (t, J = 5.4 Hz, 2H), 3.56 (t, J = 11.2 Hz, 3H), 3.19-3.03 (m, 2H), 3.03-2.77 (m, 4H), |
| | 2.70-2.52 (m, 2H), 1.21-1.17 (m, 3H). |
| 63 | 12.19 (s, 1H), 10.36 (s, 1H), 8.06 (d, J = 8.6 Hz, 1H), 7.93 (d, J = 26.1 Hz, 2H), 7.72 (d, J = 8.7 Hz, 1H), |
| | 6.83 (s, 1H), 5.32 (s, 2H), 4.25 (d, J = 2.6 Hz, 4H), 3.80 (t, J = 5.4 Hz, 2H), 3.46 (t, J = 10.3 Hz, 4H), |
| | 3.00 (d, J = 7.0 Hz, 4H), 2.73 (d, J = 10.3 Hz, 2H), 2.67 (s, 1H), 1.20 (t, J = 7.4 Hz, 3H). |
| 64 | 10.45 (s, 1H), 8.37 (s, 1H), 8.04 (d, J = 8.6 Hz, 1H), 7.96 (d, J = 2.1 Hz, 1H), 7.86 (s, 1H), 7.71 (dd, J = |
| | 9.0, 2.1 Hz, 1H), 6.83 (dq, J = 3.2, 1.6 Hz, 1H), 5.33 (s, 2H), 4.25 (d, J = 2.9 Hz, 4H), 3.80 (t, J = 5.4 Hz, |
| | 2H), 3.63 (s, 4H), 3.52-3.44 (m, 6H), 3.11 (s, 3H), 3.00 (d, J = 7.5 Hz, 2H), 2.73 (d, J = 11.0 Hz, 2H), |
| | 1.20 (t, J = 7.4 Hz, 3H). |
| 65 | 10.42 (s, 1H), 8.05 (d, J = 8.6 Hz, 1H), 7.96 (d, J = 2.2 Hz, 1H), 7.71 (dd, J = 8.7, 2.1 Hz, 1H), 7.53 (dd, |
| | J = 6.8, 2.1 Hz, 1H), 7.48 (dd, J = 6.5, 2.2 Hz, 1H), 6.85-6.79 (m, 1H), 6.26 (t, J = 6.6 Hz, 1H), 5.32 (s, |
| | 2H), 4.47 (d, J = 12.5 Hz, 1H), 4.25 (d, J = 2.9 Hz, 2H), 3.80 (t, J = 5.4 Hz, 2H), 3.52-3.38 (m, 4H), |
| | 3.23 (d, J = 13.2 Hz, 2H), 2.98 (q, J = 7.4 Hz, 2H), 2.88 (d, J = 3.1 Hz, 1H), 2.76 (d, J = 11.2 Hz, 1H), |
| | 2.64 (d, J = 10.1 Hz, 1H), 1.18 (t, J = 7.4 Hz, 3H). |
| 66 | 10.33 (s, 1H), 8.05 (d, J = 8.5 Hz, 1H), 7.96 (d, J = 2.1 Hz, 1H), 7.75-7.69 (m, 2H), 6.83 (p, J = 1.5 Hz, |
| | 1H), 5.32 (s, 2H), 4.59 (dt, J = 30.1, 4.9 Hz, 1H), 4.38 (d, J = 12.5 Hz, 1H), 4.25 (q, J = 2.8 Hz, 2H), |
| | 3.92 (d, J = 13.0 Hz, 1H), 3.80 (t, J = 5.4 Hz, 2H), 3.50 (t, J = 11.8 Hz, 4H), 3.22 (t, J = 12.9 Hz, 4H), |
| | 3.02-2.94 (m, 2H), 2.73 (d, J = 14.8 Hz, 2H), 2.09 (t, J = 8.0 Hz, 2H), 1.19 (t, J = 7.5 Hz, 3H). |
| 67 | 11.75 (s, 1H), 10.43 (s, 1H), 8.18 (d, J = 5.6 Hz, 1H), 8.05 (d, J = 8.6 Hz, 1H), 7.96 (s, 1H), 7.71 (d, J = |
| | 8.6 Hz, 1H), 7.55 (s, 1H), 7.47 (d, J = 5.6 Hz, 1H), 6.83 (s, 1H), 6.55 (s, 1H), 5.32 (s, 2H), 4.64 (d, J 12.1 |
| | Hz, 1H), 4.26 (s, 2H), 3.81 (t, J = 5.1 Hz, 2H), 3.64-3.54 (m, 4H), 3.20 (s, 2H), 3.04 (s, 1H), 3.02- |
| | 2.97 (m, 2H), 2.84 (d, J = 10.7 Hz, 1H), 2.59 (d, J = 11.1 Hz, 1H), 1.19 (t, J = 7.3 Hz, 3H). |
| 68 | 11.56 (s, 1H), 10.39 (s, 1H), 8.40 (d, J = 4.7 Hz, 1H), 8.04 (d, J = 8.6 Hz, 1H), 7.96 (d, J = 1.5 Hz, 1H), |
| | 7.81-7.61 (m, 2H), 7.14-7.09 (m, 1H), 6.83 (s, 1H), 6.64 (dd, J = 3.1, 1.8 Hz, 1H), 5.31 (s, 2H), 4.62 |
| | (s, 1H), 4.34-4.17 (m, 2H), 3.80 (t, J = 5.5 Hz, 2H), 2.99 (d, J = 7.5 Hz, 2H), 2.84 (s, 2H), 2.63 (s, 1H), |
| | 1.19 (t, J = 7.4 Hz, 2H). |
| 69 | 12.36 (s, 1H), 10.32 (s, 1H), 8.81 (s, 1H), 8.05 (d, J = 8.5 Hz, 1H), 7.96 (s, 1H), 7.75-7.64 (m, 2H), |
| | 6.83 (s, 1H), 6.58 (d, J = 3.4 Hz, 1H), 5.31 (s, 2H), 4.61 (d, J = 12.1 Hz, 1H), 4.25 (s, 2H), 3.80 (t, J = |
| | 5.1 Hz, 2H), 3.65-3.52 (m, 2H), 3.50-3.37 (m, 2H), 3.12-3.05 (m, 1H), 2.98 (d, J = 6.2 Hz, 2H), |
| | 2.86 (d, J = 10.5 Hz, 1H), 2.61 (d, J = 10.2 Hz, 1H), 1.18 (t, J = 7.3 Hz, 3H). |
| 70 | 10.40 (s, 1H), 8.24 (s, 1H), 8.05 (d, J = 8.6 Hz, 1H), 7.96 (s, 1H), 7.79 (s, 1H), 7.72 (d, J = 8.6 Hz, 1H), |
| | 6.83 (s, 1H), 5.33 (s, 2H), 4.76 (d, J = 170.6 Hz, 3H), 4.28-4.21 (m, 2H), 3.79 (t, J = 5.3 Hz, 2H), 3.46 |
| | (s, 2H), 3.16 (s, 2H), 3.03-2.97 (m, 2H), 2.89 (s, 1H), 2.74 (d, J = 10.3 Hz, 2H), 1.20 (t, J = 7.5 Hz, |
| | 3H). |
| 71 | 10.35 (s, 1H), 8.06 (d, J = 8.3 Hz, 2H), 7.96 (d, J = 1.6 Hz, 1H), 7.71 (d, J = 6.9 Hz, 1H), 7.31 (d, J = |
| | 46.2 Hz, 2H), 6.83 (s, 1H), 5.31 (s, 2H), 4.53 (d, J = 13.4 Hz, 1H), 4.25 (d, J = 2.5 Hz, 2H), 3.80 (t, J = |
| | 5.4 Hz, 2H), 3.45 (d, J = 10.7 Hz, 2H), 3.27-3.20 (m, 2H), 3.03-2.85 (m, 3H), 2.81 (s, 1H), 2.62 (d, |
| | J = 9.7 Hz, 1H), 2.54-2.52 (m, 2H), 1.19 (t, J = 7.5 Hz, 3H). |
| 72 | 10.53 (s, 1H), 10.36 (s, 1H), 8.13 (d, J = 6.9 Hz, 1H), 8.05 (d, J = 8.6 Hz, 1H), 7.96 (d, J = 1.6 Hz, 1H), |
| | 7.71 (d, J = 7.1 Hz, 1H), 6.83 (s, 1H), 6.65 (s, 1H), 6.59 (d, J = 7.0 Hz, 1H), 5.31 (s, 2H), 4.30-4.23 (m, |
| | 2H), 4.07 (s, 2H), 3.80 (t, J = 5.4 Hz, 2H), 3.54-3.43 (m, 2H), 3.29 (s, 2H), 3.12 (s, 2H), 2.99 (d, J = |
| | 7.5 Hz, 2H), 2.72 (d, J = 10.5 Hz, 2H), 2.37 (s, 3H), 1.19 (t, J = 7.5 Hz, 3H). |
| 73 | 12.55 (s, 1H), 10.39 (s, 1H), 8.05 (d, J = 8.4 Hz, 1H), 7.96 (d, J = 1.6 Hz, 1H), 7.72 (dd, J = 8.7, 1.6 Hz, |
| | 1H), 6.83 (s, 1H), 5.32 (s, 2H), 4.43 (d, J = 12.3 Hz, 1H), 4.30-4.21 (m, 2H), 3.80 (t, J = 5.5 Hz, 3H), |
| | 3.48-3.39 (m, 3H), 3.21 (d, J = 10.2 Hz, 2H), 3.14 (s, 3H), 3.06-2.91 (m, 3H), 2.79 (d, J = 11.2 Hz, |
| | 1H), 2.65 (d, J = 11.1 Hz, 1H), 1.19 (t, J = 7.5 Hz, 3H). |
| 74 | 11.07 (s, 1H), 10.37 (s, 1H), 9.19 (s, 1H), 8.52 (d, J = 5.5 Hz, 1H), 8.07 (d, J = 8.6 Hz, 1H), 7.98 (s, 1H), |
| | 7.89 (d, J = 5.5 Hz, 1H), 7.72 (d, J = 8.6 Hz, 1H), 6.84 (s, 1H), 5.33 (s, 2H), 4.26 (d, J = 2.5 Hz, 4H), |
| | 3.81 (t, J = 5.4 Hz, 2H), 3.53 (t, J = 10.4 Hz, 2H), 3.36 (s, 2H), 3.27-3.18 (m, 2H), 3.02 (dd, J = 14.7, |
| | 7.3 Hz, 2H), 2.78 (d, J = 11.2 Hz, 2H), 1.21 (t, J = 7.4 Hz, 3H). |
| 75 | 10.37 (s, 1H), 8.58 (s, 1H), 8.06 (d, J = 8.4 Hz, 1H), 7.97 (s, 1H), 7.72 (d, J = 7.0 Hz, 1H), 6.83 (s, 1H), |
| | 5.33 (s, 2H), 4.49 (s, 1H), 4.25 (d, J = 2.4 Hz, 2H), 3.80 (t, J = 5.4 Hz, 2H), 3.52 (t, J = 10.4 Hz, 3H), |
| | 3.35 (s, 5H), 3.02 (d, J = 7.7 Hz, 3H), 2.76 (s, 2H), 1.21 (dd, J = 12.9, 5.3 Hz, 3H). |
| 76 | 13.30 (s, 1H), 10.36 (s, 1H), 8.33-8.19 (m, 1H), 8.06 (d, J = 8.6 Hz, 1H), 7.96 (d, J = 1.6 Hz, 1H), 7.71 |
| | (dd, J = 8.6, 1.6 Hz, 1H), 6.83 (s, 1H), 5.31 (s, 2H), 4.47 (d, J = 12.5 Hz, 1H), 4.26 (dd, J = 9.4, 2.7 Hz, |
| | 2H), 3.80 (t, J = 5.4 Hz, 2H), 3.49-3.38 (m, 2H), 3.30 (s, 4H), 3.26-3.18 (m, 1H), 3.03-2.89 (m, 3H), |
| | 2.79 (d, J = 10.8 Hz, 1H), 2.63 (d, J = 10.8 Hz, 1H), 1.93 (s, 3H), 1.18 (t, J = 7.5 Hz, 3H). |
| 77 | 11.08 (s, 1H), 10.36 (s, 1H), 8.05 (d, J = 8.5 Hz, 1H), 7.97 (d, J = 7.3 Hz, 2H), 7.71 (d, J = 8.6 Hz, 1H), |
| | 7.30 (s, 1H), 6.83 (s, 1H), 6.68 (s, 1H), 5.31 (s, 2H), 4.25 (s, 2H), 3.84-3.74 (m, 6H), 3.53-3.42 (m, |
| | 4H), 3.11 (s, 2H), 2.99 (d, J = 7.1 Hz, 3H), 2.70 (d, J = 9.5 Hz, 2H), 1.19 (t, J = 7.4 Hz, 3H). |
| 78 | 11.96 (s, 1H), 10.36 (s, 1H), 8.06 (d, J = 8.6 Hz, 1H), 7.97 (d, J = 1.6 Hz, 1H), 7.72 (dd, J = 8.6, 1.5 Hz, |
| | 1H), 7.56 (s, 1H), 6.83 (s, 1H), 6.52 (s, 1H), 5.32 (s, 2H), 4.44 (s, 1H), 4.25 (d, J = 2.5 Hz, 2H), 3.80 (t, J = |
| | 5.4 Hz, 2H), 3.50 (dd, J = 36.1, 25.4 Hz, 3H), 3.32-3.22 (m, 4H), 2.99 (d, J = 7.3 Hz, 3H), 2.70 (dd, |
| | J = 23.6, 21.8 Hz, 2H), 1.19 (t, J = 7.5 Hz, 3H). |
| 79 | 10.65-9.96 (m, 1H), 8.06 (d, J = 8.6 Hz, 2H), 7.96 (s, 1H), 7.72 (d, J = 8.8 Hz, 1H), 6.83 (s, 1H), 6.49 |
| | (d, J = 34.9 Hz, 1H), 5.31 (s, 2H), 4.43 (d, J = 12.6 Hz, 1H), 4.25 (d, J = 2.3 Hz, 2H), 3.80 (t, J = 5.3 Hz, |
| | 2H), 3.69 (d, J = 11.9 Hz, 1H), 3.45 (t, J = 10.7 Hz, 2H), 3.27 (s, 3H), 2.97 (t, J = 10.6 Hz, 3H), 2.81 (d, |
| | J = 10.7 Hz, 1H), 2.68 (d, J = 9.9 Hz, 1H), 1.19 (t, J = 7.5 Hz, 3H). |
| 80 | 10.35 (s, 1H), 9.49 (s, 1H), 8.05 (d, J = 8.4 Hz, 1H), 7.96 (s, 1H), 7.71 (d, J = 8.5 Hz, 1H), 7.33 (s, 1H), |
| | 7.16 (d, J = 3.1 Hz, 1H), 6.82 (s, 1H), 6.78 (s, 1H), 6.33 (d, J = 3.1 Hz, 1H), 5.31 (s, 2H), 4.25 (s, 2H), |
| | 3.80 (t, J = 5.4 Hz, 2H), 3.67 (s, 3H), 3.45 (t, J = 10.2 Hz, 2H), 3.07 (s, 4H), 2.98 (d, J = 7.6 Hz, 3H), |
| | 1.24 (s, 3H), 1.18 (t, J = 7.5 Hz, 3H). |
| 81 | 13.44 (s, 1H), 10.36 (s, 1H), 9.42 (s, 1H), 8.08-8.04 (m, 2H), 7.96 (d, J = 1.4 Hz, 1H), 7.71 (dd, J = 8.7, |
| | 1.6 Hz, 1H), 6.83 (s, 1H), 5.31 (s, 2H), 4.25 (d, J = 2.5 Hz, 2H), 3.80 (t, J = 5.4 Hz, 2H), 3.50 (d, J = |
| | 36.6 Hz, 8H), 2.99 (d, J = 7.8 Hz, 4H), 1.19 (t, J = 7.5 Hz, 3H). |
| 82 | 10.57 (s, 1H), 8.29 (s, 1H), 8.03 (d, J = 8.6 Hz, 1H), 7.98-7.94 (m, 1H), 7.74-7.70 (m, 1H), 7.47 (s, |
| | 1H), 6.84 (s, 1H), 5.35 (s, 2H), 4.52 (s, 1H), 4.30-4.19 (m, 2H), 3.80 (t, J = 5.3 Hz, 2H), 3.54 (d, J = |
| | 7.0 Hz, 3H), 3.00 (d, J = 6.7 Hz, 3H), 2.83 (d, J = 9.7 Hz, 1H), 2.53 (d, J = 1.9 Hz, 4H), 1.20 (t, J = 7.4 |
| | Hz, 3H). |
| 83 | 10.35 (s, 1H), 8.14 (s, 1H), 8.06 (d, J = 8.5 Hz, 1H), 7.96 (s, 1H), 7.72 (d, J = 8.8 Hz, 1H), 6.83 (s, 1H), |
| | 6.15 (s, 1H), 5.31 (s, 2H), 4.50 (d, J = 13.6 Hz, 1H), 4.25 (s, 2H), 3.80 (t, J = 5.4 Hz, 2H), 3.45 (d, J = |
| | 10.3 Hz, 2H), 2.98 (d, J = 7.7 Hz, 2H), 2.88 (d, J = 9.4 Hz, 2H), 2.76 (d, J = 10.9 Hz, 2H), 2.58 (s, 2H), |
| | 1.19 (t, J = 7.4 Hz, 3H). |
| 84 | 11.89 (s, 1H), 10.36 (s, 1H), 8.06 (dd, J = 8.6, 6.1 Hz, 1H), 7.96 (d, J = 2.8 Hz, 2H), 7.83-7.78 (m, 1H), |
| | 7.72 (dd, J = 8.8, 2.2 Hz, 1H), 6.93 (dd, J = 4.4, 1.7 Hz, 1H), 6.83 (dd, J = 3.5, 1.9 Hz, 1H), 6.76 (dd, J = |
| | 4.4, 2.6 Hz, 1H), 5.32 (d, J = 2.1 Hz, 2H), 4.25 (d, J = 2.8 Hz, 2H), 4.09 (d, J = 12.4 Hz, 2H), 3.80 (t, J = |
| | 5.4 Hz, 2H), 3.50 (dt, J = 11.6, 5.9 Hz, 2H), 3.19-3.10 (m, 2H), 3.00 (q, J = 7.4 Hz, 2H), 2.73 (d, J = |
| | 11.0 Hz, 2H), 2.67 (q, J = 1.9 Hz, 1H), 2.33 (p, J = 1.9 Hz, 1H), 1.20 (t, J = 7.5 Hz, 3H). |
| 85 | 11.23 (s, 1H), 10.36 (s, 1H), 8.05 (d, J = 8.5 Hz, 2H), 7.96 (d, J = 1.6 Hz, 1H), 7.71 (dd, J = 8.6, 1.4 Hz, |
| | 1H), 7.55 (s, 1H), 7.23 (s, 1H), 6.83 (s, 1H), 5.31 (s, 2H), 4.25 (d, J = 1.7 Hz, 2H), 3.80 (t, J = 5.3 Hz, |
| | 2H), 3.53-3.39 (m, 4H), 3.26 (d, J = 12.4 Hz, 2H), 2.99 (td, J = 14.0, 7.5 Hz, 4H), 2.82 (d, J = 11.4 Hz, |
| | 1H), 2.63 (d, J = 10.2 Hz, 1H), 1.18 (t, J = 7.5 Hz, 3H). |
| 86 | 12.47 (s, 1H), 10.37 (s, 1H), 8.96 (s, 1H), 8.90 (s, 1H), 8.06 (d, J = 8.8 Hz, 1H), 7.97 (s, 1H), 7.72 (d, J = |
| | 8.9 Hz, 1H), 6.95 (s, 1H), 6.83 (s, 1H), 5.95-5.81 (m, 1H), 5.33 (s, 2H), 4.26 (s, 3H), 3.80 (t, J = 5.6 |
| | Hz, 2H), 3.52 (s, 6H), 3.02 (d, J = 7.8 Hz, 4H), 1.22 (d, J = 7.3 Hz, 4H). |
| 87 | 11.25 (s, 1H), 10.37 (s, 1H), 8.64 (dd, J = 4.6, 1.6 Hz, 1H), 8.32 (dd, J = 8.1, 1.5 Hz, 1H), 8.06 (d, J = |
| | 8.5 Hz, 1H), 7.97 (d, J = 1.6 Hz, 1H), 7.72 (dd, J = 8.6, 1.6 Hz, 1H), 7.48 (dd, J = 8.1, 4.6 Hz, 1H), 6.83 |
| | (s, 1H), 5.33 (s, 2H), 4.25 (d, J = 2.5 Hz, 4H), 3.80 (t, J = 5.4 Hz, 2H), 3.53 (dd, J = 11.7, 9.2 Hz, 2H), |
| | 3.23 (d, J = 11.6 Hz, 4H), 3.01 (d, J = 7.3 Hz, 2H), 2.78 (d, J = 11.3 Hz, 2H), 1.21 (t, J = 7.5 Hz, 3H). |
| 88 | 10.39 (s, 1H), 8.14 (d, J = 5.6 Hz, 1H), 8.07 (d, J = 8.5 Hz, 1H), 7.98 (d, J = 1.6 Hz, 1H), 7.73 (dd, J = |
| | 8.7, 1.7 Hz, 1H), 7.45 (s, 1H), 6.84 (s, 1H), 6.77 (d, J = 5.6 Hz, 1H), 5.33 (s, 2H), 4.39 (s, 2H), 4.26 (d, |
| | J = 2.5 Hz, 2H), 3.80 (t, J = 5.4 Hz, 2H), 3.52 (t, J = 10.5 Hz, 2H), 3.39 (s, 2H), 3.35-3.32 (m, 2H), 3.02 |
| | (dd, J = 14.3, 7.0 Hz, 2H), 2.81 (d, J = 9.5 Hz, 2H), 1.22 (t, J = 7.5 Hz, 3H). |
| 89 | 10.36 (s, 1H), 8.15 (d, J = 2.5 Hz, 1H), 8.09-8.04 (m, 1H), 7.97 (d, J = 2.2 Hz, 1H), 7.76-7.66 (m, 2H), |
| | 6.83 (p, J = 1.4 Hz, 1H), 5.32 (d, J = 2.1 Hz, 2H), 4.31 (d, J = 12.7 Hz, 1H), 4.25 (q, J = 2.9 Hz, 2H), |
| | 3.85-3.77 (m, 3H), 3.50-3.41 (m, 2H), 3.29-3.23 (m, 1H), 2.99 (dt, J = 7.7, 4.7 Hz, 2H), 2.86 (td, J = |
| | 12.3, 3.3 Hz, 1H), 2.75 (t, J = 10.1 Hz, 2H), 2.52 (d, J = 1.8 Hz, 2H), 1.19 (t, J = 7.5 Hz, 3H). |
| 90 | 13.27 (s, 1H), 10.37 (s, 1H), 8.16 (s, 1H), 8.05 (d, J = 8.6 Hz, 1H), 7.96 (d, J = 2.1 Hz, 1H), 7.90-7.84 |
| | (m, 1H), 7.71 (dd, J = 8.8, 2.2 Hz, 1H), 7.42 (d, J = 7.0 Hz, 1H), 7.23-7.17 (m, 1H), 6.82 (dd, J = 3.3, |
| | 1.7 Hz, 1H), 5.32 (s, 2H), 4.25 (d, J = 2.9 Hz, 2H), 3.80 (t, J = 5.4 Hz, 2H), 3.28 (s, 2H), 2.99 (d, J = 7.5 |
| | Hz, 2H), 2.70 (d, J = 25.2 Hz, 2H), 1.20 (t, J = 7.4 Hz, 3H). |
| 91 | 11.12 (d, J = 2.3 Hz, 1H), 8.05 (d, J = 8.5 Hz, 1H), 7.95 (d, J = 2.1 Hz, 1H), 7.71 (dd, J = 8.8, 2.2 Hz, |
| | 1H), 7.64 (d, J = 7.7 Hz, 1H), 7.37 (t, J = 2.8 Hz, 1H), 7.14 (dd, J = 7.2, 1.2 Hz, 1H), 7.06 (t, J = 7.5 Hz, |
| | 1H), 6.82 (td, J = 3.0, 1.5 Hz, 1H), 6.50 (dd, J = 3.1, 1.8 Hz, 1H), 5.31 (s, 2H), 4.25 (d, J = 2.9 Hz, 2H), |
| | 3.80 (s, 2H), 3.53-3.37 (m, 4H), 2.99 (q, J = 7.5 Hz, 2H), 2.73 (s, 2H), 1.20 (t, J = 7.4 Hz, 3H). |
| 92 | 12.47 (s, 1H), 10.36 (s, 1H), 8.06 (d, J = 8.4 Hz, 1H), 8.00-7.95 (m, 2H), 7.74-7.69 (m, 1H), 7.13 (s, |
| | 1H), 6.83 (s, 1H), 5.89 (d, J = 1.6 Hz, 1H), 5.32 (s, 2H), 4.78-3.89 (m, 4H), 3.80 (t, J = 5.2 Hz, 2H), |
| | 3.65-3.36 (m, 4H), 3.15-2.84 (m, 4H), 2.76-2.64 (m, 2H), 2.16 (s, 3H), 1.20 (t, J = 7.6 Hz, 3H). |

### Example 93: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzamide

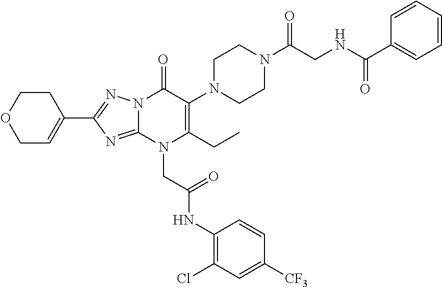 [SMILES: CCc1c(N2CCN(C(=O)CNC(=O)c3ccccc3)CC2)c(=O)n2nc(C3=CCOCC3)nc2n1CC(=O)Nc1ccc(C(F)(F)F)cc1Cl]

Method C: A mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (10 mg, 0.02 mmol, Example 38), benzoic acid (2.34 mg, 0.02 mmol), HATU (8.51 mg, 0.02 mmol) and K₂CO₃ (6.62 mg, 0.05 mmol) in DMF (3 mL) was stirred at r.t. overnight. The reaction mixture was filtered. The filtrate was concentrated under reduced pressure. The residue was purified by Prep-HPLC on a C18 column eluting with MeCN/H₂O (30-50%, with 0.1% FA) to afford the title compound (5.65 mg, 48% yield) as a white solid. ¹H NMR (400 MHZ, DMSO-d₆)₈ ppm 10.36 (s, 1H), 8.60 (t, J=5.7 Hz, 1H), 8.06 (d, J=8.4 Hz, 1H), 7.97 (s, 1H), 7.92-7.86 (m, 2H), 7.72 (d, J=8.9 Hz, 1H), 7.55 (t, J=7.3 Hz, 1H), 7.49 (t, J=7.3 Hz, 2H), 6.84 (s, 1H), 5.32 (s, 2H), 4.39 (d, J=11.6 Hz, 1H), 4.26 (d, J=2.5 Hz, 2H), 4.20 (d, J=5.8 Hz, 2H), 3.97 (d, J=11.8 Hz, 1H), 3.81 (t, J=5.4 Hz, 2H), 3.50 (d, J=10.8 Hz, 2H), 3.26 (s, 1H), 3.00 (d, J=7.4 Hz, 2H), 2.85-2.67 (m, 5H), 1.20 (t, J=7.5 Hz, 3H). LCMS calc. for C₃₄H₃₅ClF₃N₈O₅ [M+H]⁺: m/z=727.2; Found: 727.4.

The following compounds listed in Table 3 were prepared by using an appropriate acid and Example 38 as the methods analogous to those described for Method C.

TABLE 3
Preparations of Examples (Ex)

| Ex | Acid | Structure |
|---|---|---|
| 94 | 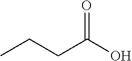 | 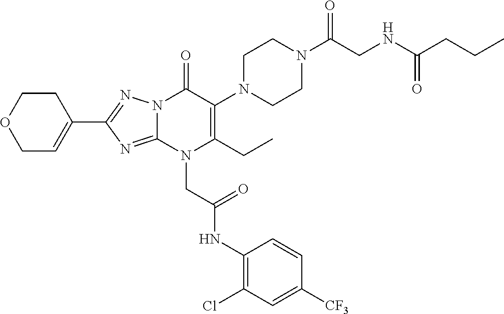 [SMILES: CCCC(=O)NCC(=O)N1CCN(c2c(CC)n(CC(=O)Nc3ccc(C(F)(F)F)cc3Cl)c3nc(C4=CCOCC4)nn3c2=O)CC1] |
| 95 | 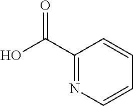 | 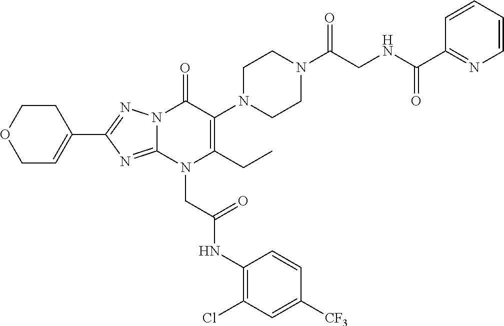 [SMILES: CCc1c(N2CCN(C(=O)CNC(=O)c3ccccn3)CC2)c(=O)n2nc(C3=CCOCC3)nc2n1CC(=O)Nc1ccc(C(F)(F)F)cc1Cl] |
| 96 | 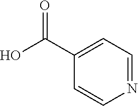 | 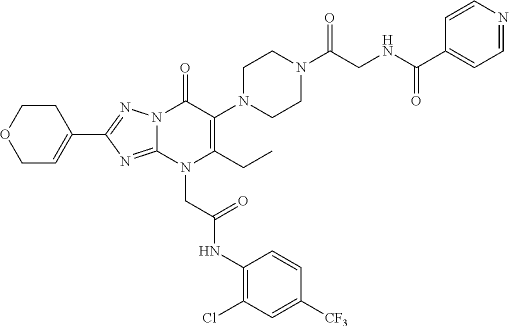 [SMILES: CCc1c(N2CCN(C(=O)CNC(=O)c3ccncc3)CC2)c(=O)n2nc(C3=CCOCC3)nc2n1CC(=O)Nc1ccc(C(F)(F)F)cc1Cl] |
| 97 | 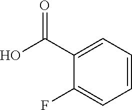 | 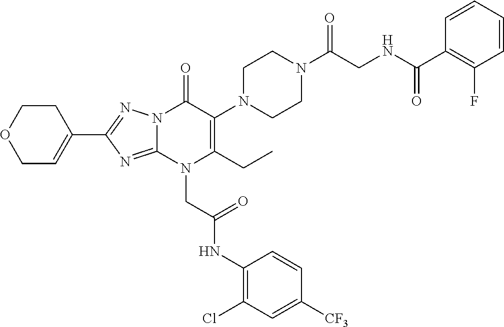 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1Cl] |
| 98 | 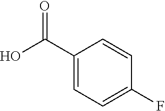 | 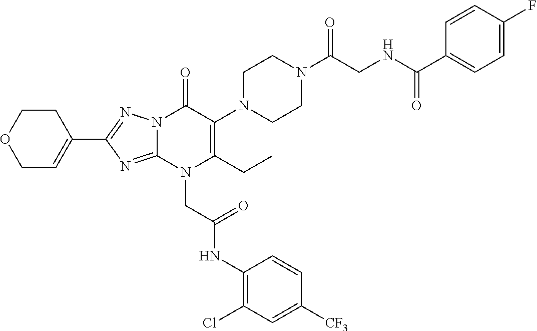 [SMILES: CCc1c(N2CCN(C(=O)CNC(=O)c3ccc(F)cc3)CC2)c(=O)n2nc(C3=CCOCC3)nc2n1CC(=O)Nc1ccc(C(F)(F)F)cc1Cl] |
| 99 | 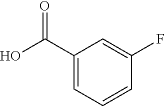 | 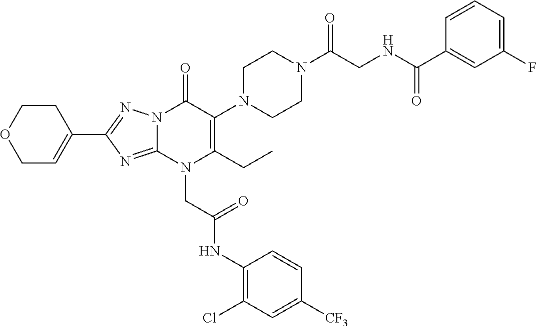 [SMILES: CCc1c(N2CCN(C(=O)CNC(=O)c3cccc(F)c3)CC2)c(=O)n2nc(C3=CCOCC3)nc2n1CC(=O)Nc1ccc(C(F)(F)F)cc1Cl] |
| 100 | 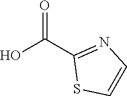 | 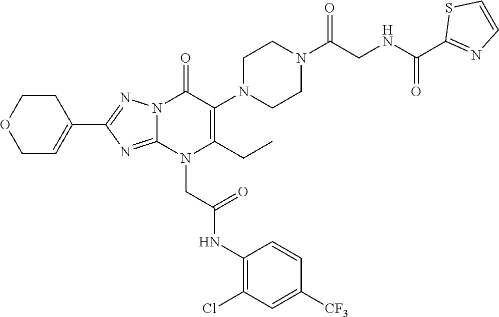 [SMILES: CCc1c(N2CCN(C(=O)CNC(=O)c3nccs3)CC2)c(=O)n2nc(C3=CCOCC3)nc2n1CC(=O)Nc1ccc(C(F)(F)F)cc1Cl] |
| 101 | 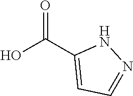 | 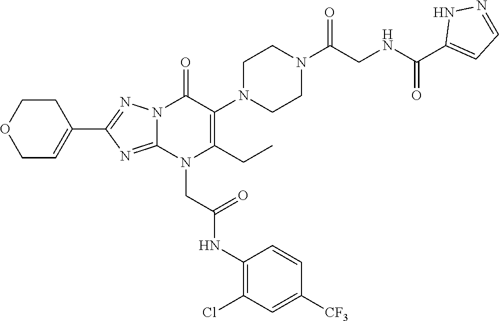 [SMILES: CCc1c(N2CCN(C(=O)CNC(=O)c3ccn[nH]3)CC2)c(=O)n2nc(C3=CCOCC3)nc2n1CC(=O)Nc1ccc(C(F)(F)F)cc1Cl] |
| 102 | 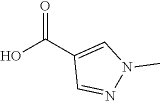 | 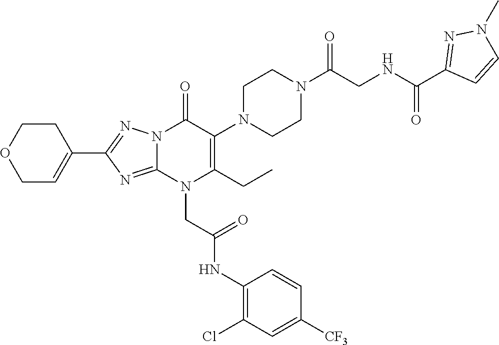 [SMILES: CCc1c(N2CCN(C(=O)CNC(=O)c3ccn(C)n3)CC2)c(=O)n2nc(C3=CCOCC3)nc2n1CC(=O)Nc1ccc(C(F)(F)F)cc1Cl] |
| 103 | 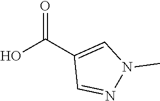 | 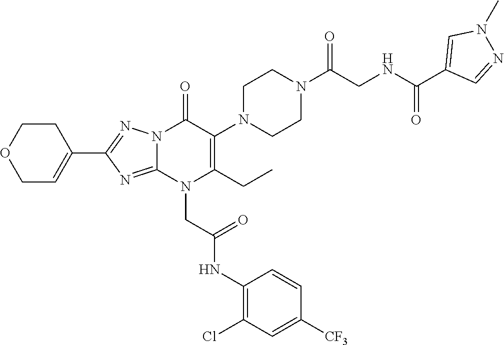 [SMILES: CCc1c(N2CCN(C(=O)CNC(=O)c3cnn(C)c3)CC2)c(=O)n2nc(C3=CCOCC3)nc2n1CC(=O)Nc1ccc(C(F)(F)F)cc1Cl] |
| 104 | 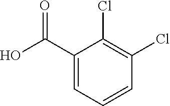 [SMILES: O=C(O)c1cccc(Cl)c1Cl] | 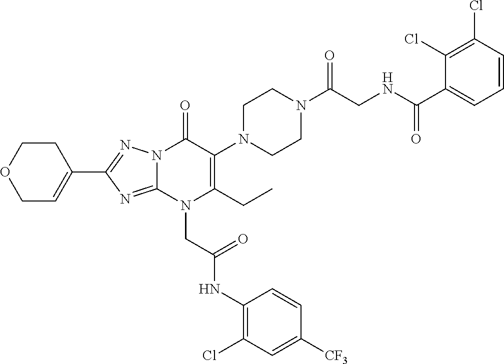 [SMILES: CCc1c(N2CCN(C(=O)CNC(=O)c3cccc(Cl)c3Cl)CC2)c(=O)n2nc(C3=CCOCC3)nc2n1CC(=O)Nc1ccc(C(F)(F)F)cc1Cl] |
| 105 | 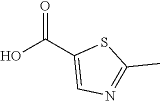 | 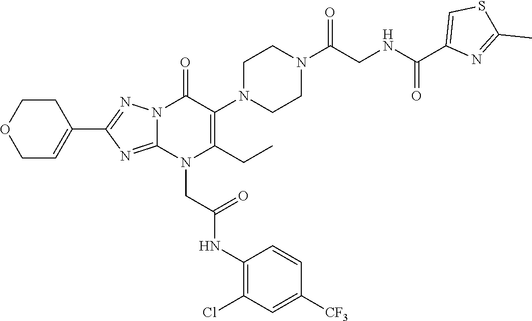 [SMILES: CCc1c(N2CCN(C(=O)CNC(=O)c3csc(C)n3)CC2)c(=O)n2nc(C3=CCOCC3)nc2n1CC(=O)Nc1ccc(C(F)(F)F)cc1Cl] |
| 106 | 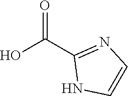 | 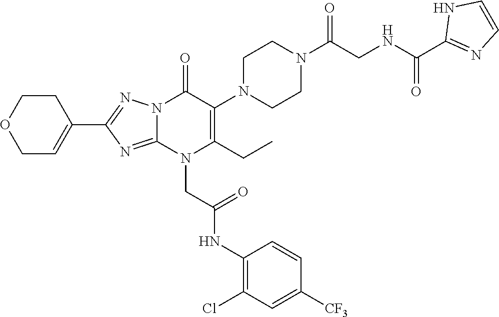 [SMILES: CCc1c(N2CCN(C(=O)CNC(=O)c3ncc[nH]3)CC2)c(=O)n2nc(C3=CCOCC3)nc2n1CC(=O)Nc1ccc(C(F)(F)F)cc1Cl] |
| 107 | 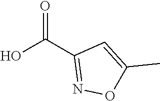 | 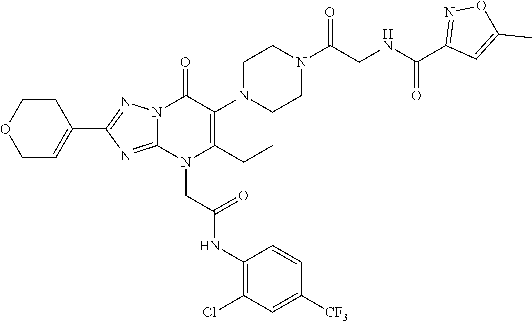 [SMILES: CCc1c(N2CCN(C(=O)CNC(=O)c3cc(C)on3)CC2)c(=O)n2nc(C3=CCOCC3)nc2n1CC(=O)Nc1ccc(C(F)(F)F)cc1Cl] |
| 108 | INT C25 | 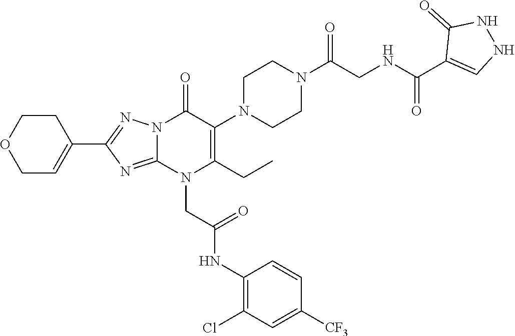 [SMILES: CCc1c(N2CCN(C(=O)CNC(=O)c3c[nH][nH]c3=O)CC2)c(=O)n2nc(C3=CCOCC3)nc2n1CC(=O)Nc1ccc(C(F)(F)F)cc1Cl] |
| 109 | 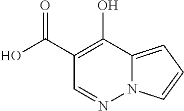 [SMILES: O=C(O)c1cnn2cccc2c1O] | 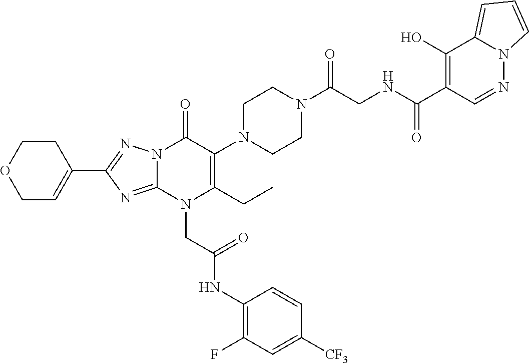 [SMILES: CCc1c(N2CCN(C(=O)CNC(=O)c3cnn4cccc4c3O)CC2)c(=O)n2nc(C3=CCOCC3)nc2n1CC(=O)Nc1ccc(C(F)(F)F)cc1F] |
| 110 | 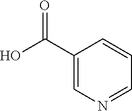 | 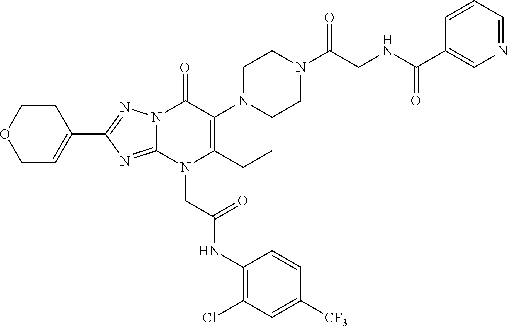 [SMILES: CCc1c(N2CCN(C(=O)CNC(=O)c3cccnc3)CC2)c(=O)n2nc(C3=CCOCC3)nc2n1CC(=O)Nc1ccc(C(F)(F)F)cc1Cl] |
| 111 | 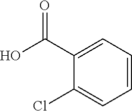 | 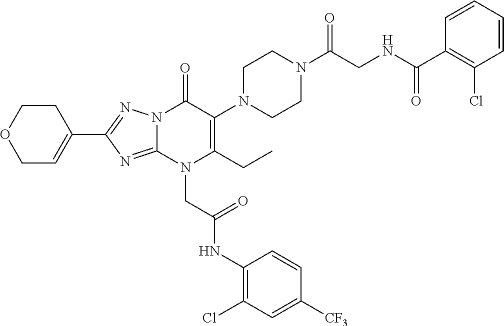 [SMILES: CCc1c(N2CCN(C(=O)CNC(=O)c3ccccc3Cl)CC2)c(=O)n2nc(C3=CCOCC3)nc2n1CC(=O)Nc1ccc(C(F)(F)F)cc1Cl] |
| 112 | 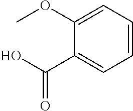 | 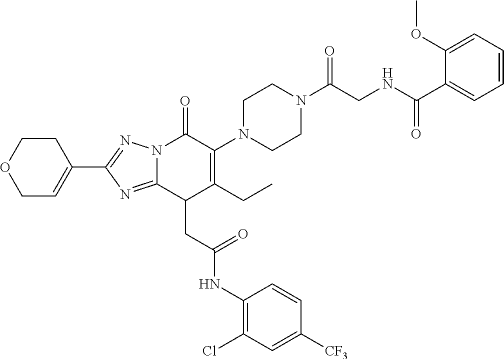 [SMILES: CCC1=C(N2CCN(C(=O)CNC(=O)c3ccccc3OC)CC2)C(=O)n2nc(C3=CCOCC3)nc2C1CC(=O)Nc1ccc(C(F)(F)F)cc1Cl] |
| 113 | 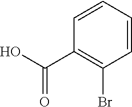 | 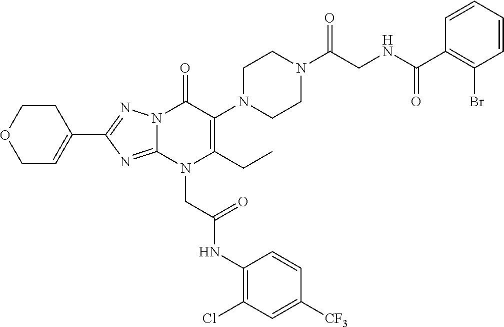 [SMILES: CCc1c(N2CCN(C(=O)CNC(=O)c3ccccc3Br)CC2)c(=O)n2nc(C3=CCOCC3)nc2n1CC(=O)Nc1ccc(C(F)(F)F)cc1Cl] |
| 114 | 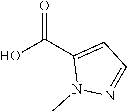 | 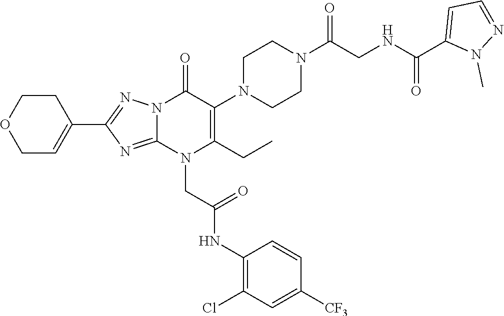 [SMILES: CCc1c(N2CCN(C(=O)CNC(=O)c3ccnn3C)CC2)c(=O)n2nc(C3=CCOCC3)nc2n1CC(=O)Nc1ccc(C(F)(F)F)cc1Cl] |
| 115 | 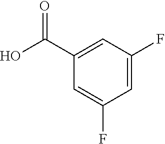 [SMILES: O=C(O)c1cc(F)cc(F)c1] | 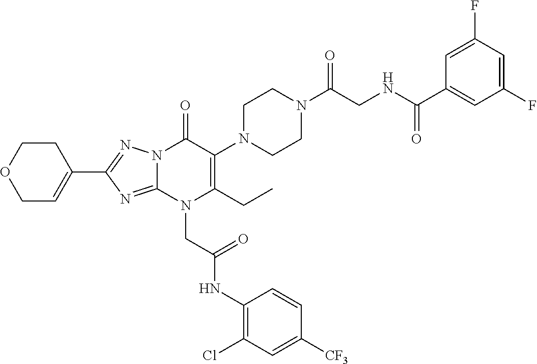 [SMILES: CCc1c(N2CCN(C(=O)CNC(=O)c3cc(F)cc(F)c3)CC2)c(=O)n2nc(C3=CCOCC3)nc2n1CC(=O)Nc1ccc(C(F)(F)F)cc1Cl] |
| 116 | 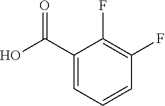 [SMILES: O=C(O)c1cccc(F)c1F] | 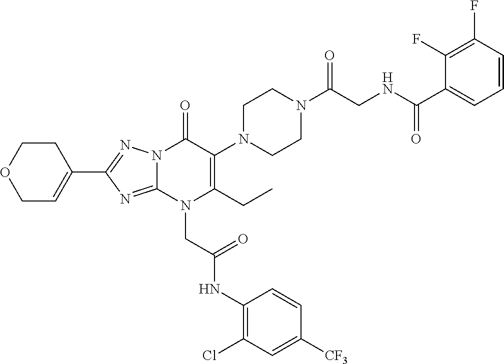 [SMILES: CCc1c(N2CCN(C(=O)CNC(=O)c3cccc(F)c3F)CC2)c(=O)n2nc(C3=CCOCC3)nc2n1CC(=O)Nc1ccc(C(F)(F)F)cc1Cl] |
| 117 | 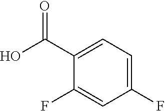 [SMILES: O=C(O)c1ccc(F)cc1F] | 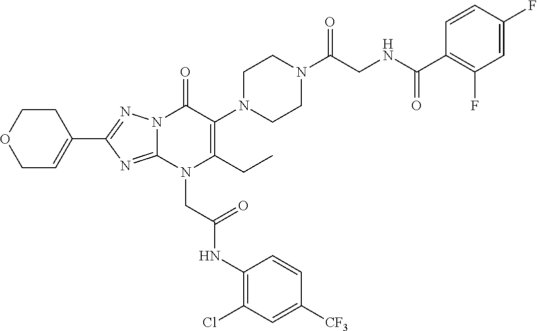 [SMILES: CCc1c(N2CCN(C(=O)CNC(=O)c3ccc(F)cc3F)CC2)c(=O)n2nc(C3=CCOCC3)nc2n1CC(=O)Nc1ccc(C(F)(F)F)cc1Cl] |
| 118 | 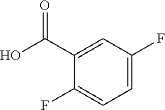 [SMILES: O=C(O)c1cc(F)ccc1F] | 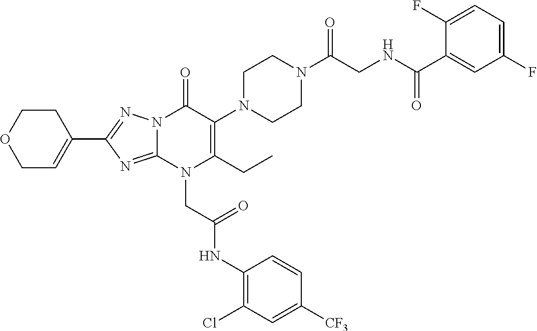 [SMILES: CCc1c(N2CCN(C(=O)CNC(=O)c3cc(F)ccc3F)CC2)c(=O)n2nc(C3=CCOCC3)nc2n1CC(=O)Nc1ccc(C(F)(F)F)cc1Cl] |
| 119 | 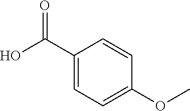 | 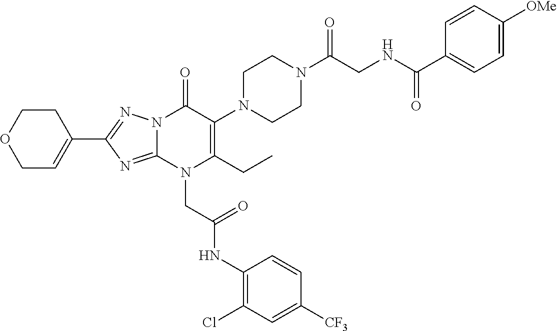 [SMILES: CCc1c(N2CCN(C(=O)CNC(=O)c3ccc(OC)cc3)CC2)c(=O)n2nc(C3=CCOCC3)nc2n1CC(=O)Nc1ccc(C(F)(F)F)cc1Cl] |
| 120 | INT C27 | 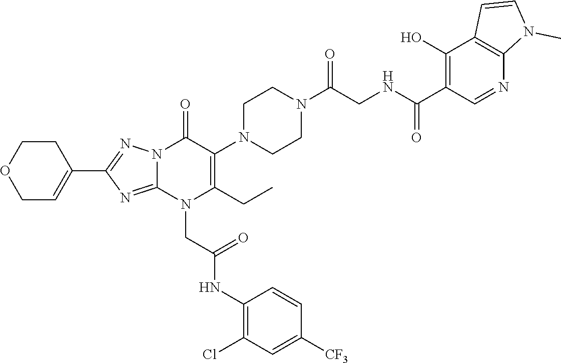 [SMILES: CCc1c(N2CCN(C(=O)CNC(=O)c3cnc4c(ccn4C)c3O)CC2)c(=O)n2nc(C3=CCOCC3)nc2n1CC(=O)Nc1ccc(C(F)(F)F)cc1Cl] |
| 121 | 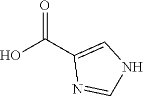 | 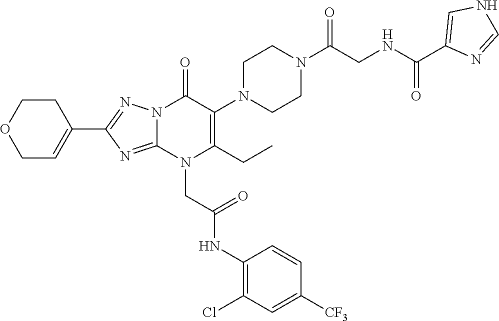 [SMILES: CCc1c(N2CCN(C(=O)CNC(=O)c3c[nH]cn3)CC2)c(=O)n2nc(C3=CCOCC3)nc2n1CC(=O)Nc1ccc(C(F)(F)F)cc1Cl] |
| 122 | 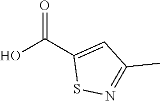 | 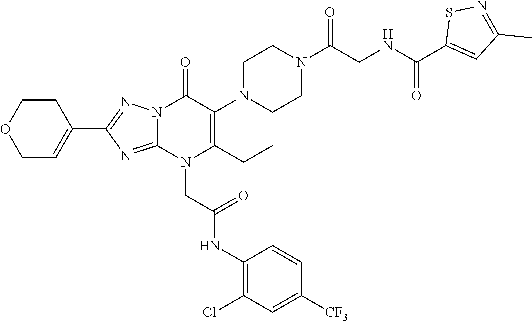 [SMILES: CCc1c(N2CCN(C(=O)CNC(=O)c3cc(C)ns3)CC2)c(=O)n2nc(C3=CCOCC3)nc2n1CC(=O)Nc1ccc(C(F)(F)F)cc1Cl] |
| 123 | 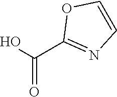 | 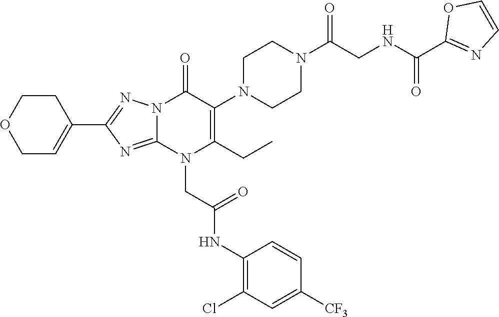 [SMILES: CCc1c(N2CCN(C(=O)CNC(=O)c3ncco3)CC2)c(=O)n2nc(C3=CCOCC3)nc2n1CC(=O)Nc1ccc(C(F)(F)F)cc1Cl] |
| 124 | 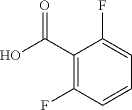 [SMILES: O=C(O)c1c(F)cccc1F] | 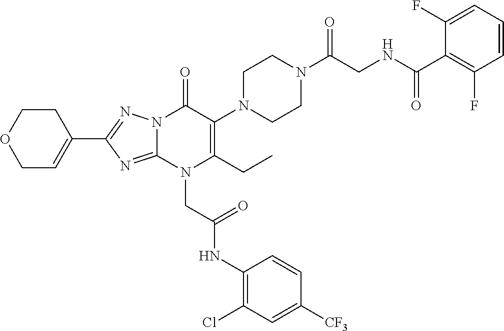 [SMILES: CCc1c(N2CCN(C(=O)CNC(=O)c3c(F)cccc3F)CC2)c(=O)n2nc(C3=CCOCC3)nc2n1CC(=O)Nc1ccc(C(F)(F)F)cc1Cl] |
| 125 | 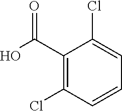 [SMILES: O=C(O)c1c(Cl)cccc1Cl] | 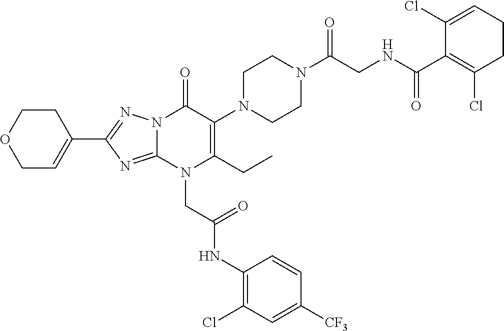 [SMILES: CCc1c(N2CCN(C(=O)CNC(=O)C3=C(Cl)CCC=C3Cl)CC2)c(=O)n2nc(C3=CCOCC3)nc2n1CC(=O)Nc1ccc(C(F)(F)F)cc1Cl] |
| 126 | 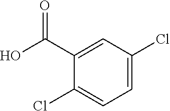 [SMILES: O=C(O)c1cc(Cl)ccc1Cl] | 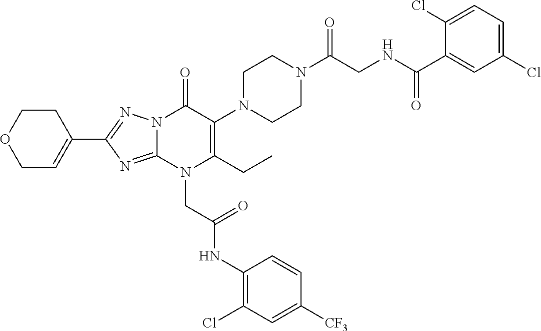 [SMILES: CCc1c(N2CCN(C(=O)CNC(=O)c3cc(Cl)ccc3Cl)CC2)c(=O)n2nc(C3=CCOCC3)nc2n1CC(=O)Nc1ccc(C(F)(F)F)cc1Cl] |
| 127 | 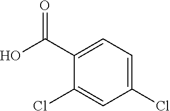 [SMILES: O=C(O)c1ccc(Cl)cc1Cl] | 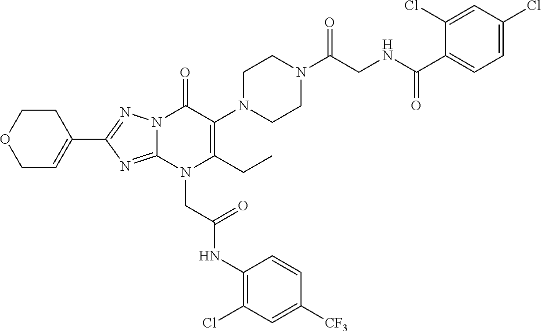 [SMILES: CCc1c(N2CCN(C(=O)CNC(=O)c3ccc(Cl)cc3Cl)CC2)c(=O)n2nc(C3=CCOCC3)nc2n1CC(=O)Nc1ccc(C(F)(F)F)cc1Cl] |
| 128 | 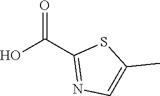 | 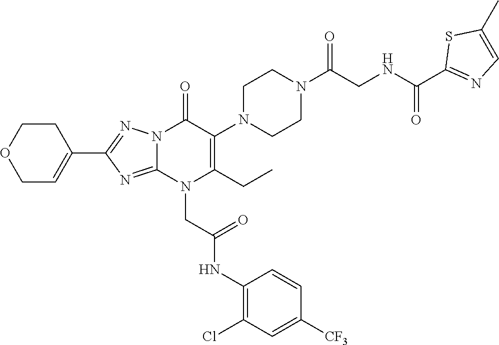 [SMILES: CCc1c(N2CCN(C(=O)CNC(=O)c3ncc(C)s3)CC2)c(=O)n2nc(C3=CCOCC3)nc2n1CC(=O)Nc1ccc(C(F)(F)F)cc1Cl] |
| 129 | 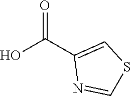 | 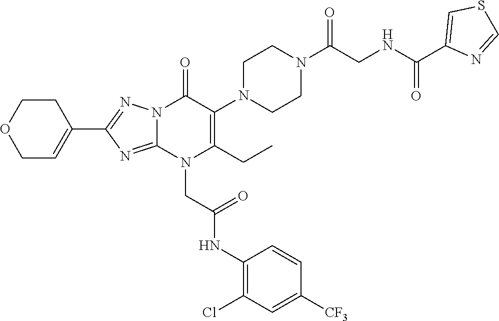 [SMILES: CCc1c(N2CCN(C(=O)CNC(=O)c3cscn3)CC2)c(=O)n2nc(C3=CCOCC3)nc2n1CC(=O)Nc1ccc(C(F)(F)F)cc1Cl] |
| 130 | 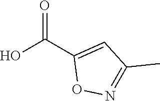 | 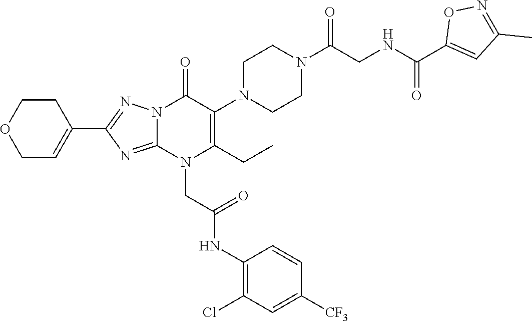 [SMILES: CCc1c(N2CCN(C(=O)CNC(=O)c3cc(C)no3)CC2)c(=O)n2nc(C3=CCOCC3)nc2n1CC(=O)Nc1ccc(C(F)(F)F)cc1Cl] |
| 131 | 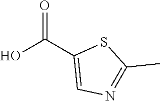 | 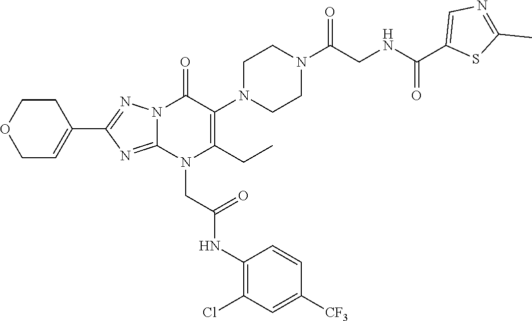 [SMILES: CCc1c(N2CCN(C(=O)CNC(=O)c3cnc(C)s3)CC2)c(=O)n2nc(C3=CCOCC3)nc2n1CC(=O)Nc1ccc(C(F)(F)F)cc1Cl] |
| 132 | 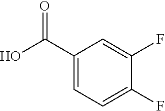 [SMILES: O=C(O)c1ccc(F)c(F)c1] | 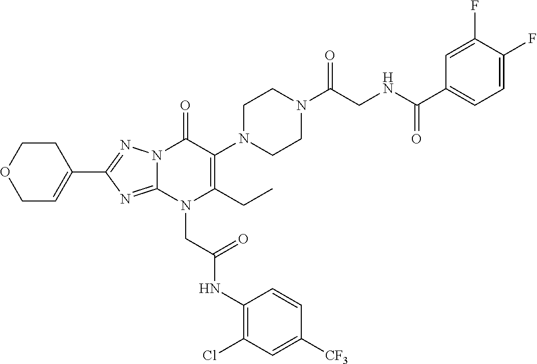 [SMILES: CCc1c(N2CCN(C(=O)CNC(=O)c3ccc(F)c(F)c3)CC2)c(=O)n2nc(C3=CCOCC3)nc2n1CC(=O)Nc1ccc(C(F)(F)F)cc1Cl] |

Preparations of Examples (Ex)

| | | LCMS: |
|---|---|---|
| | | Cacl./ |
| Ex | Name | Found |
| 94 | N-(2-(4-(4-(2-((2-Chloro-4- | [M + H]⁺: |
| | (trifluoromethyl)phenyl)amino)-2- | 693.2/ |
| | oxoethyl)-2-(3,6-dihydro-2H-pyran- | 693.2 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)butyramide | |
| 95 | N-(2-(4-(4-(2-((2-Chloro-4- | [M + H]⁺: |
| | (trifluoromethyl)phenyl)amino)-2- | 728.2/ |
| | oxoethyl)-2-(3,6-dihydro-2H-pyran- | 728.2 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)picolinamide | |
| 96 | N-(2-(4-(4-(2-((2-Chloro-4- | [M + H]⁺: |
| | (trifluoromethyl)phenyl)amino)-2- | 728.2/ |
| | oxoethyl)-2-(3,6-dihydro-2H-pyran- | 728.2 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)isonicotinamide | |
| 97 | N-(2-(4-(4-(2-((2-Chloro-4- | [M + H]⁺: |
| | (trifluoromethyl)phenyl)amino)-2- | 745.2/ |
| | oxoethyl)-2-(3,6-dihydro-2H-pyran- | 745.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2- | |
| | fluorobenzamide | |
| 98 | N-(2-(4-(4-(2-((2-Chloro-4- | [M + H]⁺: |
| | (trifluoromethyl)phenyl)amino)-2- | 745.2/ |
| | oxoethyl)-2-(3,6-dihydro-2H-pyran- | 745.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-4- | |
| | fluorobenzamide | |
| 99 | N-(2-(4-(4-(2-((2-Chloro-4- | [M + H]⁺: |
| | (trifluoromethyl)phenyl)amino)-2- | 745.2/ |
| | oxoethyl)-2-(3,6-dihydro-2H-pyran- | 745.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3- | |
| | fluorobenzamide | |
| 100 | N-(2-(4-(4-(2-((2-Chloro-4- | [M + H]⁺: |
| | (trifluoromethyl)phenyl)amino)-2- | 734.2/ |
| | oxoethyl)-2-(3,6-dihydro-2H-pyran- | 734.1 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)thiazole-2-carboxamide | |
| 101 | N-(2-(4-(4-(2-((2-Chloro-4 | [M + H]⁺: |
| | (trifluoromethyl)phenyl)amino)-2- | 717.2/ |
| | oxoethyl)-2-(3,6-dihydro-2H-pyran- | 717.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1H- | |
| | pyrazole-5-carboxamide | |
| 102 | N-(2-(4-(4-(2-((2-Chloro-4- | [M + H]⁺: |
| | (trifluoromethyl)phenyl)amino)-2- | 731.2/ |
| | oxoethyl)-2-(3,6-dihydro-2H-pyran- | 731.0 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1- | |
| | methyl-1H-pyrazole-3-carboxamide | |
| 103 | N-(2-(4-(4-(2-((2-Chloro-4- | [M + H]⁺: |
| | (trifluoromethyl)phenyl)amino)-2- | 731.2/ |
| | oxoethyl)-2-(3,6-dihydro-2H-pyran- | 731.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1- | |
| | methyl-1H-pyrazole-4-carboxamide | |
| 104 | 2,3-Dichloro-N-(2-(4-(4-(2-((2- | [M + H]⁺: |
| | chloro-4- | 795.2/ |
| | (trifluoromethyl)phenyl)amino)-2- | 795.1 |
| | oxoethyl)-2-(3,6-dihydro-2H-pyran- | |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 105 | N-(2-(4-(4-(2-((2-Chloro-4- | [M + H]⁺: |
| | (trifluoromethyl)phenyl)amino)-2- | 748.2/ |
| | oxoethyl)-2-(3,6-dihydro-2H-pyran- | 748.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2- | |
| | methylthiazole-4-carboxamide | |
| 106 | N-(2-(4-(4-(2-((2-Chloro-4- | [M + H]⁺: |
| | (trifluoromethyl)phenyl)amino)-2- | 717.2/ |
| | oxoethyl)-2-(3,6-dihydro-2H-pyran- | 717.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1H- | |
| | imidazole-2-carboxamide | |
| 107 | N-(2-(4-(4-(2-((2-Chloro-4- | [M + H]⁺: |
| | (trifluoromethyl)phenyl)amino)-2- | 732.2/ |
| | oxoethyl)-2-(3,6-dihydro-2H-pyran- | 732.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-5- | |
| | methylisoxazole-3-carboxamide | |
| 108 | N-(2-(4-(4-(2-((2-Chloro-4- | [M + H]⁺: |
| | (trifluoromethyl)phenyl)amino)-2- | 733.2/ |
| | oxoethyl)-2-(3,6-dihydro-2H-pyran- | 733.5 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3-oxo- | |
| | 2,3-dihydro-1H-pyrazole-4- | |
| | carboxamide | |
| 109 | N-(2-(4-(4-(2-((2-Chloro-4- | [M + H]⁺: |
| | (trifluoromethyl)phenyl)amino)-2- | 783.2/ |
| | oxoethyl)-2-(3,6-dihydro-2H-pyran- | 783.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-4- | |
| | hydroxypyrrolo[1,2-b]pyridazine-3- | |
| | carboxamide | |
| 110 | N-(2-(4-(4-(2-((2-Chloro-4- | [M + H]⁺: |
| | (trifluoromethyl)phenyl)amino)-2- | 728.2/ |
| | oxoethyl)-2-(3,6-dihydro-2H-pyran- | 728.4 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)nicotinamide | |
| 111 | 2-Chloro-N-(2-(4-(4-(2-((2-chloro-4- | [M + H]⁺: |
| | (trifluoromethyl)phenyl)amino)-2- | 761.2/ |
| | oxoethyl)-2-(3,6-dihydro-2H-pyran- | 761.3 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 112 | N-(2-(4-(4-(2-((2-Chloro-4- | [M + H]⁺: |
| | (trifluoromethyl)phenyl)amino)-2- | 757.2/ |
| | oxoethyl)-2-(3,6-dihydro-2H-pyran- | 757.6 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2- | |
| | methoxybenzamide | |
| 113 | 2-Bromo-N-(2-(4-(4-(2-((2-chloro-4- | [M + H]⁺: |
| | (trifluoromethyl)phenyl)amino)-2- | 805.1/ |
| | oxoethyl)-2-(3,6-dihydro-2H-pyran- | 805.2 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 114 | N-(2-(4-(4-(2-((2-Chloro-4- | [M + H]⁺: |
| | (trifluoromethyl)phenyl)amino)-2- | 731.2/ |
| | oxoethyl)-2-(3,6-dihydro-2H-pyran- | 731.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1- | |
| | methyl-1H-pyrazole-5-carboxamide | |
| 115 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3,5- | |
| | difluorobenzamide | |
| 116 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2,3- | |
| | difluorobenzamide | |
| 117 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.5 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2,4- | |
| | difluorobenzamide | |
| 118 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.1 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2,5- | |
| | difluorobenzamide | |
| 119 | N-(2-(4-(4-(2-((2-Chloro-4- | [M + H]⁺: |
| | (trifluoromethyl)phenyl)amino)-2- | 757.2/ |
| | oxoethyl)-2-(3,6-dihydro-2H-pyran- | 757.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-4- | |
| | methoxybenzamide | |
| 120 | N-(2-(4-(4-(2-((2-Chloro-4- | [M + H]⁺: |
| | (trifluoromethyl)phenyl)amino)-2- | 797.2/ |
| | oxoethyl)-2-(3,6-dihydro-2H-pyran- | 797.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-4- | |
| | hydroxy-1-methyl-1H-pyrrolo[2,3- | |
| | b]pyridine-5-carboxamide | |
| 121 | N-(2-(4-(4-(2-((2-Chloro-4- | [M + H]⁺: |
| | (trifluoromethyl)phenyl)amino)-2- | 717.2/ |
| | oxoethyl)-2-(3,6-dihydro-2H-pyran- | 717.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-1H- | |
| | imidazole-4-carboxamide | |
| 122 | N-(2-(4-(4-(2-((2-Chloro-4- | [M + H]⁺: |
| | (trifluoromethyl)phenyl)amino)-2- | 748.2/ |
| | oxoethyl)-2-(3,6-dihydro-2H-pyran- | 748.1 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3- | |
| | methylisothiazole-5-carboxamide | |
| 123 | N-(2-(4-(4-(2-((2-Chloro-4- | [M + H]⁺: |
| | (trifluoromethyl)phenyl)amino)-2- | 718.2/ |
| | oxoethyl)-2-(3,6-dihydro-2H-pyran- | 718.2 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)oxazole-2-carboxamide | |
| 124 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.1 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2,6- | |
| | difluorobenzamide | |
| 125 | 2,6-Dichloro-N-(2-(4-(4-(2-((2- | [M + H]⁺: |
| | chloro-4- | 795.2/ |
| | (trifluoromethyl)phenyl)amino)-2- | 795.1 |
| | oxoethyl)-2-(3,6-dihydro-2H-pyran- | |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 126 | 2,5-Dichloro-N-(2-(4-(4-(2-((2- | [M + H]⁺: |
| | chloro-4- | 795.2/ |
| | (trifluoromethyl)phenyl)amino)-2- | 795.1 |
| | oxoethyl)-2-(3,6-dihydro-2H-pyran- | |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 127 | 2,4-Dichloro-N-(2-(4-(4-(2-((2- | [M + H]⁺: |
| | chloro-4- | 795.2/ |
| | (trifluoromethyl)phenyl)amino)-2- | 795.1 |
| | oxoethyl)-2-(3,6-dihydro-2H-pyran- | |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)benzamide | |
| 128 | N-(2-(4-(4-(2-((2-Chloro-4- | [M + H]⁺: |
| | (trifluoromethyl)phenyl)amino)-2- | 748.2/ |
| | oxoethyl)-2-(3,6-dihydro-2H-pyran- | 748.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-5- | |
| | methylthiazole-2-carboxamide | |
| 129 | N-(2-(4-(4-(2-((2-Chloro-4- | [M + H]⁺: |
| | (trifluoromethyl)phenyl)amino)-2- | 734.2/ |
| | oxoethyl)-2-(3,6-dihydro-2H-pyran- | 734.0 |
| | 4-yl)-5-ethyl-7-oxo-4,7-dihydro- | |
| | [1,2,4]triazolo[1,5-a]pyrimidin-6- | |
| | yl)piperazin-1-yl)-2- | |
| | oxoethyl)thiazole-4-carboxamide | |
| 130 | N-(2-(4-(4-(2-((2-Chloro-4- | [M + H]⁺: |
| | (trifluoromethyl)phenyl)amino)-2- | 732.2/ |
| | oxoethyl)-2-(3,6-dihydro-2H-pyran- | 732.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3- | |
| | methylisoxazole-5-carboxamide | |
| 131 | N-(2-(4-(4-(2-((2-Chloro-4- | [M + H]⁺: |
| | (trifluoromethyl)phenyl)amino)-2- | 748.2/ |
| | oxoethyl)-2-(3,6-dihydro-2H-pyran- | 748.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-2- | |
| | methylthiazole-5-carboxamide | |
| 132 | N-(2-(4-(4-(2-((2-Chloro-4- | [M + H]⁺: |
| | (trifluoromethyl)phenyl)amino)-2- | 763.2/ |
| | oxoethyl)-2-(3,6-dihydro-2H-pyran- | 763.2 |
| | 4-yl)-5-ethyl-7-oxo-4,7-dihydro- | |
| | [1,2,4]triazolo[1,5-a]pyrimidin-6- | |
| | yl)piperazin-1-yl)-2-oxoethyl)-3,4- | |
| | difluorobenzamide | |

TABLE 4
¹H NMR of Examples (Ex)

| Ex | ¹H NMR (400 MHz, DMSO-d₆) δ ppm |
|---|---|
| 94 | 10.36 (s, 1H), 8.11-8.02 (m, 1H), 8.02-7.88 (m, 2H), 7.72 (dd, J = 8.7, 2.2 Hz, 1H), 6.86-6.80 (m, |
| | 1H), 5.32 (s, 2H), 4.37 (d, J = 12.5 Hz, 1H), 4.25 (q, J = 2.9 Hz, 2H), 3.99 (dd, J = 13.1, 5.3 Hz, 2H), |
| | 3.89-3.76 (m, 3H), 3.46 (t, J = 11.5 Hz, 2H), 3.20 (t, J = 12.0 Hz, 2H), 2.98 (d, J = 7.5 Hz, 2H), 2.82- |
| | 2.76 (m, 1H), 2.70 (d, J = 11.2 Hz, 3H), 2.13 (t, J = 7.3 Hz, 2H), 1.54 (p, J = 7.3 Hz, 2H), 1.23-1.14 |
| | (m, 3H), 0.88 (t, J = 7.4 Hz, 3H). |
| 95 | 10.34 (s, 1H), 8.85 (t, J = 5.2 Hz, 1H), 8.69 (dt, J = 4.8, 1.4 Hz, 1H), 8.07 (dt, J = 7.7, 1.2 Hz, 2H), 8.02 |
| | (td, J = 7.6, 1.7 Hz, 1H), 7.96 (d, J = 2.0 Hz, 1H), 7.72 (dd, J = 8.9, 2.1 Hz, 1H), 7.64 (ddd, J = 7.4, 4.8, |
| | 1.5 Hz, 1H), 6.83 (p, J = 1.4 Hz, 1H), 5.32 (s, 2H), 4.45-4.29 (m, 2H), 4.25 (q, J = 2.8 Hz, 2H), 4.20 (d, |
| | J = 16.6 Hz, 1H), 3.91 (d, J = 12.7 Hz, 1H), 3.80 (t, J = 5.5 Hz, 2H), 3.56-3.40 (m, 2H), 3.24 (d, J = 11.9 |
| | Hz, 1H), 3.00 (q, J = 7.3 Hz, 2H), 2.83 (t, J = 12.1 Hz, 1H), 2.74 (d, J = 11.3 Hz, 2H), 2.52 (d, |
| | J = 1.8 Hz, 2H), 1.20 (t, J = 7.4 Hz, 3H). |
| 96 | 10.36 (s, 1H), 8.94 (t, J = 5.8 Hz, 1H), 8.78-8.71 (m, 2H), 8.07 (dd, J = 8.6, 6.1 Hz, 1H), 7.97 (d, |
| | J = 2.0 Hz, 1H), 7.82-7.77 (m, 2H), 7.74 (d, J = 2.1 Hz, 1H), 6.85-6.80 (m, 1H), 5.33 (d, J = 2.1 Hz, 2H), |
| | 4.39 (d, J = 12.4 Hz, 1H), 4.24 (dt, J = 10.8, 4.0 Hz, 4H), 3.96 (d, J = 12.7 Hz, 1H), 3.80 (t, J = 5.6 Hz, |
| | 2H), 3.50 (d, J = 11.3 Hz, 2H), 3.25 (d, J = 12.1 Hz, 1H), 3.03-2.95 (m, 2H), 2.81 (s, 1H), 2.73 (d, |
| | J = 11.2 Hz, 2H), 2.52 (d, J = 1.8 Hz, 2H), 1.20 (t, J = 7.4 Hz, 3H). |
| 97 | 10.36 (s, 1H), 8.32 (d, J = 5.1 Hz, 1H), 8.07 (dd, J = 8.6, 6.1 Hz, 1H), 7.97 (d, J = 2.1 Hz, 1H), 7.79- |
| | 7.70 (m, 2H), 7.57 (tdd, J = 7.4, 6.2, 1.9 Hz, 1H), 7.36-7.29 (m, 2H), 6.83 (tt, J = 2.9, 1.4 Hz, 1H), 5.33 |
| | (d, J = 2.2 Hz, 2H), 4.40 (d, J = 12.6 Hz, 1H), 4.29-4.16 (m, 4H), 3.91 (d, J = 12.7 Hz, 1H), 3.80 (t, J = 5.4 |
| | Hz, 2H), 3.56-3.39 (m, 2H), 3.25 (t, J = 12.2 Hz, 1H), 3.00 (q, J = 7.7, 7.1 Hz, 2H), 2.81 (q, J = 8.4, |
| | 5.1 Hz, 1H), 2.73 (d, J = 11.2 Hz, 2H), 2.52 (d, J = 1.9 Hz, 2H), 1.20 (t, J = 7.5 Hz, 3H). |
| 98 | 10.36 (s, 1H), 8.65 (t, J = 5.7 Hz, 1H), 8.07 (dd, J = 8.6, 6.0 Hz, 1H), 7.96 (td, J = 5.6, 2.8 Hz, 3H), 7.72 |
| | (dd, J = 8.8, 2.1 Hz, 1H), 7.36-7.28 (m, 2H), 6.83 (p, J = 1.5 Hz, 1H), 5.33 (d, J = 2.1 Hz, 2H), 4.39 (d, |
| | J = 12.4 Hz, 1H), 4.25 (q, J = 2.9 Hz, 2H), 4.19 (d, J = 5.7 Hz, 2H), 3.96 (d, J = 12.8 Hz, 1H), 3.80 (t, |
| | J = 5.4 Hz, 2H), 3.56-3.37 (m, 2H), 3.24 (d, J = 11.9 Hz, 1H), 3.00 (q, J = 7.4 Hz, 2H), 2.82 (d, J = 12.5 |
| | Hz, 1H), 2.73 (d, J = 11.2 Hz, 2H), 2.52 (d, J = 1.9 Hz, 2H), 1.20 (t, J = 7.5 Hz, 3H). |
| 99 | 10.37 (s, 1H), 8.75 (t, J = 5.7 Hz, 1H), 8.07 (dd, J = 8.6, 6.1 Hz, 1H), 7.97 (d, J = 2.1 Hz, 1H), 7.74 (ddd, |
| | J = 11.0, 8.3, 1.8 Hz, 2H), 7.68 (ddd, J = 10.0, 2.7, 1.5 Hz, 1H), 7.55 (td, J = 8.0, 5.9 Hz, 1H), 7.45- |
| | 7.37 (m, 1H), 6.83 (t, J = 1.6 Hz, 1H), 5.33 (d, J = 2.1 Hz, 2H), 4.38 (d, J = 12.3 Hz, 1H), 4.23 (dq, J = 19.3, |
| | 2.6 Hz, 4H), 3.96 (d, J = 12.7 Hz, 1H), 3.80 (t, J = 5.4 Hz, 2H), 3.50 (d, J = 11.1 Hz, 1H), 3.38 (d, |
| | J = 3.1 Hz, 2H), 3.31-3.21 (m, 2H), 3.00 (d, J = 7.6 Hz, 2H), 2.80 (s, 1H), 2.73 (d, J = 11.2 Hz, 2H), |
| | 1.20 (t, J = 7.5 Hz, 3H). |
| 100 | 10.39 (s, 1H), 8.69 (t, J = 5.6 Hz, 1H), 8.11-8.03 (m, 3H), 7.98 (d, J = 2.0 Hz, 1H), 7.73 (dd, J = 8.6, |
| | 2.1 Hz, 1H), 6.84 (p, J = 1.5 Hz, 1H), 5.33 (s, 2H), 4.39 (d, J = 12.5 Hz, 1H), 4.29 (d, J = 6.0 Hz, 1H), |
| | 4.26 (d, J = 2.7 Hz, 2H), 4.20 (d, J = 4.3 Hz, 1H), 3.91 (d, J = 12.6 Hz, 1H), 3.80 (t, J = 5.6 Hz, 2H), |
| | 3.51 (t, J = 11.3 Hz, 2H), 3.42 (s, 1H), 3.23 (d, J = 12.2 Hz, 2H), 3.00 (q, J = 7.3 Hz, 2H), 2.85-2.79 |
| | (m, 1H), 2.73 (d, J = 11.3 Hz, 2H), 1.20 (t, J = 7.4 Hz, 3H). |
| 101 | 13.28 (s, 1H), 10.38 (s, 1H), 8.07 (d, J = 8.6 Hz, 2H), 7.97 (d, J = 2.0 Hz, 1H), 7.84 (s, 1H), 7.73 (dd, J = 8.8, |
| | 2.1 Hz, 1H), 6.84 (dq, J = 3.2, 1.6 Hz, 1H), 6.66 (s, 1H), 5.33 (s, 2H), 4.40 (d, J = 12.4 Hz, 1H), 4.27- |
| | 4.24 (m, 2H), 4.18 (dd, J = 16.5, 3.9 Hz, 2H), 3.91 (d, J = 12.7 Hz, 1H), 3.80 (t, J = 5.6 Hz, 2H), 3.56- |
| | 3.44 (m, 2H), 3.41 (s, 1H), 3.22 (d, J = 11.7 Hz, 2H), 2.99 (d, J = 7.6 Hz, 2H), 2.83 (d, J = 11.0 Hz, 1H), |
| | 2.73 (d, J = 11.2 Hz, 2H). |
| 102 | 10.36 (s, 1H), 8.07 (dd, J = 8.5, 6.0 Hz, 1H), 7.97 (d, J = 2.0 Hz, 1H), 7.95 (d, J = 5.3 Hz, 1H), 7.79 (d, |
| | J = 2.3 Hz, 1H), 7.72 (dd, J = 8.7, 2.1 Hz, 1H), 6.83 (dq, J = 2.9, 1.4 Hz, 1H), 6.64 (d, J = 2.2 Hz, 1H), |
| | 5.33 (d, J = 2.1 Hz, 2H), 4.39 (d, J = 12.5 Hz, 1H), 4.26 (q, J = 2.9 Hz, 2H), 4.21-4.05 (m, 2H), 3.92 (s, |
| | 3H), 3.84-3.76 (m, 2H), 3.50 (t, J = 11.5 Hz, 1H), 3.41 (d, J = 9.6 Hz, 2H), 3.23 (t, J = 12.1 Hz, 1H), |
| | 2.99 (q, J = 7.3 Hz, 2H), 2.83 (d, J = 10.5 Hz, 1H), 2.72 (d, J = 11.3 Hz, 2H), 1.20 (t, J = 7.5 Hz, 3H). |
| 103 | 10.37 (s, 1H), 8.20 (t, J = 5.7 Hz, 1H), 8.16 (s, 1H), 8.07 (d, J = 8.7 Hz, 1H), 7.97 (d, J = 2.1 Hz, 1H), |
| | 7.87 (s, 1H), 7.72 (dd, J = 8.8, 2.1 Hz, 1H), 6.83 (tt, J = 2.9, 1.5 Hz, 1H), 5.33 (s, 2H), 4.38 (d, J = 12.3 |
| | Hz, 1H), 4.26 (q, J = 2.9 Hz, 2H), 4.13 (d, J = 5.7 Hz, 2H), 3.94 (d, J = 12.8 Hz, 1H), 3.86 (s, 3H), 3.83- |
| | 3.78 (m, 2H), 3.49 (t, J = 11.6 Hz, 1H), 3.22 (d, J = 12.1 Hz, 2H), 2.99 (q, J = 7.3 Hz, 2H), 2.80 (d, |
| | J = 9.4 Hz, 1H), 2.72 (d, J = 11.3 Hz, 2H), 1.19 (t, J = 7.4 Hz, 3H). |
| 104 | 10.36 (s, 1H), 8.68 (t, J = 5.8 Hz, 1H), 8.06 (d, J = 8.5 Hz, 1H), 7.97 (s, 1H), 7.72 (dd, J = 5.5,4.0 Hz, |
| | 2H), 7.47-7.43 (m, 2H), 6.83 (s, 1H), 5.33 (s, 2H), 4.40 (d, J = 12.1 Hz, 1H), 4.25 (d, J = 2.5 Hz, 2H), |
| | 4.19 (dd, J = 18.5, 5.6 Hz, 2H), 3.93 (d, J = 11.9 Hz, 1H), 3.80 (t, J = 5.4 Hz, 2H), 3.52 (t, J = 10.5 Hz, |
| | 1H), 3.40 (d, J = 10.9 Hz, 1H), 3.24 (d, J = 11.8 Hz, 2H), 3.05-2.95 (m, 2H), 2.76 (dt, J = 28.8, 15.0 Hz, |
| | 4H), 1.20 (t, J = 7.5 Hz, 3H). |
| 105 | 10.36 (s, 1H), 8.28 (t, J = 5.2 Hz, 1H), 8.13 (s, 1H), 8.06 (d, J = 8.5 Hz, 1H), 7.97 (s, 1H), 7.73 (d, |
| | J = 8.6 Hz, 1H), 6.83 (s, 1H), 5.32 (s, 2H), 4.39 (d, J = 11.5 Hz, 1H), 4.26 (d, J = 2.6 Hz, 3H), 4.17 (d, |
| | J = 5.2 Hz, 1H), 3.89 (d, J = 11.8 Hz, 1H), 3.80 (t, J = 5.5 Hz, 2H), 3.51 (t, J = 10.8 Hz, 2H), 3.24 (t, J = 11.9 |
| | Hz, 2H), 2.99 (d, J = 7.5 Hz, 2H), 2.82 (t, J = 9.9 Hz, 2H), 2.73 (s, 5H), 1.20 (t, J = 7.5 Hz, 3H). |
| 106 | 13.07 (s, 1H), 10.38 (s, 1H), 8.26 (t, J = 5.4 Hz, 1H), 8.07 (d, J = 8.5 Hz, 1H), 7.98 (s, 1H), 7.73 (d, |
| | J = 7.1 Hz, 1H), 7.29 (s, 1H), 7.07 (s, 1H), 6.84 (s, 1H), 5.33 (s, 2H), 4.40 (d, J = 11.7 Hz, 1H), 4.25 (t, |
| | J = 5.9 Hz, 3H), 4.18-4.09 (m, 1H), 3.90 (d, J = 12.7 Hz, 1H), 3.80 (t, J = 5.5 Hz, 2H), 3.48 (dd, J = 35.3, |
| | 24.0 Hz, 3H), 3.23 (d, J = 11.9 Hz, 1H), 3.00 (d, J = 7.6 Hz, 2H), 2.91-2.59 (m, 4H), 1.20 (t, J = 7.5 |
| | Hz, 3H). |
| 107 | 10.36 (s, 1H), 8.56 (t, J = 5.6 Hz, 1H), 8.06 (d, J = 8.5 Hz, 1H), 7.97 (d, J = 1.3 Hz, 1H), 7.76-7.69 (m, |
| | 1H), 6.84 (s, 1H), 6.57 (d, J = 0.8 Hz, 1H), 5.32 (s, 2H), 4.38 (d, J = 11.6 Hz, 1H), 4.23 (dd, J = 22.7, 3.8 |
| | Hz, 3H), 4.14 (t, J = 8.3 Hz, 1H), 3.91 (d, J = 13.2 Hz, 1H), 3.81 (t, J = 5.4 Hz, 2H), 3.51 (t, J = 11.3 Hz, |
| | 1H), 3.41 (s, 2H), 3.30-3.16 (m, 2H), 2.99 (d, J = 7.4 Hz, 2H), 2.81 (t, J = 11.2 Hz, 1H), 2.72 (d, |
| | J = 11.0 Hz, 2H), 2.48 (s, 3H), 1.20 (t, J = 7.5 Hz, 3H). |
| 108 | 12.15 (s, 1H), 11.00 (s, 1H), 10.37 (s, 1H), 8.06 (d, J = 8.5 Hz, 1H), 7.97 (s, 1H), 7.85 (s, 1H), 7.73 (d, |
| | J = 7.4 Hz, 1H), 6.83 (s, 1H), 5.32 (s, 2H), 4.39 (d, J = 12.8 Hz, 1H), 4.25 (s, 3H), 4.19 (s, 2H), 3.83-3.79 |
| | (m, 4H), 3.23 (s, 3H), 2.99 (d, J = 7.2 Hz, 2H), 2.76 (dd, J = 26.5, 11.0 Hz, 4H), 1.20 (t, J = 7.5 Hz, 3H). |
| 109 | 10.37 (s, 1H), 8.43 (s, 1H), 8.07 (d, J = 8.5 Hz, 1H), 7.97 (d, J = 2.1 Hz, 1H), 7.71 (dt, J = 16.0, 8.0 Hz, |
| | 2H), 6.83 (s, 1H), 6.75 (s, 1H), 6.64 (s, 1H), 5.33 (s, 2H), 4.40 (d, J = 12.4 Hz, 1H), 4.25 (q, J = 3.2 Hz, |
| | 4H), 3.93 (d, J = 12.7 Hz, 1H), 3.80 (t, J = 5.5 Hz, 2H), 3.57-3.38 (m, 3H), 3.27-3.16 (m, 2H), 3.00 |
| | (d, J = 7.7 Hz, 2H), 2.83 (d, J = 11.9 Hz, 1H), 2.73 (d, J = 11.1 Hz, 2H), 2.42-2.50 (m, 2H), 1.20 (t, |
| | J = 7.4 Hz, 3H). |
| 110 | 10.37 (s, 1H), 9.06 (d, J = 1.5 Hz, 1H), 8.87 (t, J = 5.7 Hz, 1H), 8.74 (dd, J = 4.8, 1.4 Hz, 1H), 8.27- |
| | 8.23 (m, 1H), 8.07 (d, J = 8.5 Hz, 1H), 7.97 (d, J = 1.5 Hz, 1H), 7.73 (dd, J = 8.7, 1.7 Hz, 1H), 7.55 (dd, |
| | J = 7.9, 4.8 Hz, 1H), 6.84 (s, 1H), 5.33 (s, 2H), 4.39 (d, J = 11.8 Hz, 1H), 4.27-4.22 (m, 4H), 3.97 (d, |
| | J = 12.4 Hz, 1H), 3.81 (t, J = 5.5 Hz, 2H), 3.57-3.47 (m, 2H), 3.27 (t, J = 11.2 Hz, 2H), 3.00 (dd, J = 14.4, |
| | 7.0 Hz, 2H), 2.81 (dd, J = 14.7, 7.9 Hz, 2H), 2.73 (d, J = 11.0 Hz, 2H), 1.20 (t, J = 7.5 Hz, 3H). |
| 111 | 10.36 (s, 1H), 8.53 (t, J = 5.6 Hz, 1H), 8.06 (d, J = 8.6 Hz, 1H), 7.97 (s, 1H), 7.73 (d, J = 8.8 Hz, 1H), |
| | 7.54-7.39 (m, 4H), 6.84 (s, 1H), 5.33 (s, 2H), 4.40 (d, J = 12.1 Hz, 1H), 4.26 (s, 2H), 4.18 (dd, J = 14.9, |
| | 5.5 Hz, 2H), 3.94 (d, J = 11.9 Hz, 1H), 3.80 (t, J = 5.3 Hz, 2H), 3.52 (t, J = 11.2 Hz, 1H), 3.41 (d, J = 10.2 |
| | Hz, 2H), 3.24 (d, J = 13.0 Hz, 1H), 3.00 (d, J = 6.9 Hz, 2H), 2.78 (dd, J = 37.8, 12.0 Hz, 4H), 1.20 |
| | (t, J = 7.5 Hz, 3H). |
| 112 | 10.34 (s, 1H), 8.74 (t, J = 3.9 Hz, 1H), 8.07 (d, J = 8.7 Hz, 1H), 7.98-7.93 (m, 2H), 7.73 (d, J = 8.7 Hz, |
| | 1H), 7.53 (t, J = 7.7 Hz, 1H), 7.20 (d, J = 8.3 Hz, 1H), 7.08 (t, J = 7.4 Hz, 1H), 6.84 (s, 1H), 5.33 (s, 2H), |
| | 4.43 (d, J = 12.3 Hz, 1H), 4.34-4.19 (m, 4H), 3.96 (s, 3H), 3.87 (d, J = 9.7 Hz, 1H), 3.80 (t, J = 5.3 Hz, |
| | 2H), 3.50 (d, J = 15.2 Hz, 1H), 3.44-3.38 (m, 2H), 3.24 (d, J = 11.8 Hz, 1H), 3.00 (d, J = 7.0 Hz, 2H), |
| | 2.88-2.69 (m, 4H), 1.20 (t, J = 7.4 Hz, 3H). |
| 113 | 8.49 (dd, J = 26.2, 20.7 Hz, 2H), 8.06 (d, J = 8.4 Hz, 1H), 7.97 (s, 1H), 7.72 (d, J = 7.4 Hz, 1H), 7.66 (d, |
| | J = 7.8, 1H), 7.51-7.43 (m, 2H), 7.41-7.34 (m, 1H), 6.83 (s, 1H), 5.32 (s, 2H), 4.40 (d, J = 13.0 Hz, 1H), |
| | 4.25 (d, J = 2.4 Hz, 2H), 4.17 (dd, J = 14.5, 5.8 Hz, 2H), 3.94 (d, J = 11.4 Hz, 1H), 3.80 (t, J = 5.5 Hz, |
| | 2H), 3.52 (t, J = 12.0 Hz, 1H), 3.39 (s, 1H), 3.24 (d, J = 14.6 Hz, 2H), 3.00 (d, J = 7.6 Hz, 2H), 2.86-2.65 |
| | (m, 4H), 1.20 (t, J = 7.5 Hz, 3H). |
| 114 | 10.36 (s, 1H), 8.61 (t, J = 5.6 Hz, 1H), 8.06 (d, J = 8.3 Hz, 1H), 7.97 (s, 1H), 7.73 (d, J = 8.6 Hz, 1H), |
| | 7.48 (d, J = 1.9 Hz, 1H), 6.91 (d, J = 2.0 Hz, 1H), 6.83 (s, 1H), 5.32 (s, 2H), 4.39 (d, J = 7.9 Hz, 1H), |
| | 4.26 (s, 2H), 4.16 (s, 2H), 4.06 (s, 3H), 3.95 (d, J = 12.0 Hz, 1H), 3.81 (t, J = 5.5 Hz, 2H), 3.56-3.39 |
| | (m, 2H), 3.24 (d, J = 10.2 Hz, 2H), 3.00 (d, J = 7.2 Hz, 2H), 2.88-2.68 (m, 4H), 1.20 (t, J = 7.5 Hz, |
| | 3H). |
| 115 | 10.36 (s, 1H), 8.85 (t, J = 5.8 Hz, 1H), 8.06 (d, J = 8.5 Hz, 1H), 7.97 (s, 1H), 7.72 (d, J = 7.0 Hz, 1H), |
| | 7.65-7.56 (m, 2H), 7.53-7.45 (m, 1H), 6.83 (s, 1H), 5.26 (d, J = 52.4 Hz, 2H), 4.38 (d, J = 11.4 Hz, |
| | 1H), 4.30-4.15 (m, 4H), 3.96 (d, J = 11.8 Hz, 1H), 3.81 (t, J = 5.5 Hz, 2H), 3.52 (s, 1H), 3.39 (s, 2H), |
| | 3.26 (s, 1H), 3.00 (d, J = 7.5 Hz, 2H), 2.73 (dd, J = 31.6, 20.5 Hz, 4H), 1.20 (t, J = 7.5 Hz, 3H). |
| 116 | 10.36 (s, 1H), 8.52 (d, J = 2.6 Hz, 1H), 8.06 (d, J = 8.4 Hz, 1H), 7.97 (d, J = 1.6 Hz, 1H), 7.72 (dd, J = 8.7, |
| | 1.6 Hz, 1H), 7.60 (ddd, J = 12.3, 8.9, 7.3 Hz, 1H), 7.50 (dd, J = 7.8, 6.2 Hz, 1H), 7.35-7.29 (m, |
| | 1H), 6.83 (s, 1H), 5.32 (s, 2H), 4.40 (d, J = 12.4 Hz, 1H), 4.23 (dd, J = 20.7, 4.2 Hz, 4H), 3.92 (d, J = 12.2 |
| | Hz, 1H), 3.80 (t, J = 5.4 Hz, 2H), 3.46 (d, J = 48.1 Hz, 2H), 3.24 (d, J = 10.6 Hz, 4H), 3.00 (d, J = 7.6 |
| | Hz, 2H), 2.83-2.71 (m, 2H), 1.20 (t, J = 7.5 Hz, 3H). |
| 117 | 10.37 (s, 1H), 8.33 (dd, J = 9.4, 4.2 Hz, 1H), 8.06 (d, J = 9.2 Hz, 1H), 7.97 (s, 1H), 7.82 (dd, J = 15.4, |
| | 8.3 Hz, 1H), 7.72 (d, J = 7.1 Hz, 1H), 7.39 (t, J = 10.5 Hz, 1H), 7.22 (t, J = 8.1 Hz, 1H), 6.83 (s, 1H), |
| | 5.32 (s, 2H), 4.39 (d, J = 12.5 Hz, 1H), 4.25 (s, 2H), 4.24-4.17 (m, 2H), 3.91 (d, J = 12.6 Hz, 1H), 3.80 |
| | (t, J = 5.6 Hz, 2H), 3.57-3.44 (m, 4H), 3.00 (d, J = 8.1 Hz, 2H), 2.76-2.66 (m, 4H), 1.20 (t, J = 7.5 |
| | Hz, 3H). |
| 118 | 10.36 (s, 1H), 8.49-8.40 (m, 1H), 8.06 (d, J = 8.4 Hz, 1H), 7.96 (s, 1H), 7.72 (d, J = 8.5 Hz, 1H), 7.50 |
| | (ddd, J = 8.6, 5.6, 2.9 Hz, 1H), 7.42 (ddq, J = 14.0, 9.3, 4.8 Hz, 2H), 6.83 (s, 1H), 5.32 (s, 2H), 4.40 (d, |
| | J = 11.7 Hz, 1H), 4.25 (s, 3H), 4.21 (d, J = 5.7 Hz, 1H), 3.91 (d, J = 12.5 Hz, 1H), 3.80 (t, J = 5.4 Hz, 2H), |
| | 3.50 (d, J = 10.0 Hz, 1H), 3.39 (s, 2H), 3.25 (s, 2H), 3.00 (d, J = 7.6 Hz, 2H), 2.82 (t, J = 10.9 Hz, 1H), |
| | 2.73 (d, J = 11.5 Hz, 2H), 1.20 (t, J = 7.4 Hz, 3H). |
| 119 | 10.36 (s, 1H), 8.45 (t, J = 5.6 Hz, 1H), 8.07 (d, J = 8.6 Hz, 1H), 7.97 (d, J = 1.5 Hz, 1H), 7.87 (d, J = 8.9 |
| | Hz, 2H), 7.72 (dd, J = 8.7, 1.7 Hz, 1H), 7.02 (d, J = 8.9 Hz, 2H), 6.84 (s, 1H), 5.33 (s, 2H), 4.39 (d, |
| | J = 11.3 Hz, 1H), 4.26 (d, J = 2.4 Hz, 2H), 4.17 (d, J = 5.6 Hz, 2H), 3.97 (d, J = 12.6 Hz, 1H), 3.82 (s, 3H), |
| | 3.80 (d, J = 5.5 Hz, 2H), 3.57-3.46 (m, 1H), 3.40 (d, J = 11.7 Hz, 2H), 3.29-3.20 (m, 1H), 3.00 (d, |
| | J = 7.5 Hz, 2H), 2.87-2.69 (m, 4H), 1.20 (t, J = 7.5 Hz, 3H). |
| 120 | 10.37 (s, 1H), 8.49 (s, 1H), 8.06 (t, J = 6.6 Hz, 1H), 7.97 (s, 1H), 7.72 (d, J = 7.1 Hz, 1H), 7.08 (s, 1H), |
| | 6.83 (s, 1H), 6.51 (d, J = 9.7 Hz, 1H), 5.33 (s, 2H), 4.41 (d, J = 12.9 Hz, 1H), 4.26 (d, J = 9.0 Hz, 4H), |
| | 3.92 (d, J = 12.2 Hz, 1H), 3.79 (dd, J = 15.6, 10.1 Hz, 6H), 3.50 (d, J = 10.5 Hz, 2H), 3.24 (d, J = 11.6 |
| | Hz, 2H), 3.00 (d, J = 7.5 Hz, 3H), 2.82 (d, J = 14.3 Hz, 1H), 2.73 (d, J = 10.8 Hz, 2H), 1.20 (t, J = 7.5 |
| | Hz, 3H). |
| 121 | 12.50 (s, 1H), 10.37 (s, 1H), 8.06 (d, J = 8.5 Hz, 1H), 7.94 (m, 2H), 7.72 (m, 2H), 7.64 (s, 1H), 6.83 (s, |
| | 1H), 5.32 (s, 2H), 4.40 (d, J = 12.3 Hz, 1H), 4.29-4.08 (m, 4H), 3.88 (d, J = 12.1 Hz, 1H), 3.80 (t, |
| | J = 5.4 Hz, 2H), 3.47 (m, 4H), 3.23 (m, 2H), 2.99 (d, J = 7.4 Hz, 2H), 2.72 (d, J = 10.6 Hz, 2H), 1.20 (t, |
| | J = 7.5 Hz, 3H). |
| 122 | 10.37 (s, 1H), 8.99 (t, J = 5.9 Hz, 1H), 8.06 (d, J = 8.5 Hz, 1H), 7.98 (d, J = 1.6 Hz, 1H), 7.75-7.71 (m, |
| | 2H), 6.83 (s, 1H), 5.33 (s, 2H), 4.38 (d, J = 12.5 Hz, 1H), 4.28-4.17 (m, 4H), 3.93 (d, J = 12.2 Hz, 1H), |
| | 3.81 (t, J = 5.4 Hz, 2H), 3.55-3.34 (m, 3H), 3.30-3.20 (m, 2H), 2.99 (d, J = 7.3 Hz, 2H), 2.80 (t, J = 11.3 |
| | Hz, 1H), 2.72 (d, J = 11.2 Hz, 2H), 2.48 (s, 3H), 1.20 (t, J = 7.5 Hz, 3H). |
| 123 | 10.37 (s, 1H), 8.75 (t, J = 5.7 Hz, 1H), 8.35 (s, 1H), 8.06 (d, J = 8.6 Hz, 1H), 7.97 (s, 1H), 7.72 (d, |
| | J = 8.7 Hz, 1H), 7.49 (s, 1H), 6.84 (s, 1H), 5.32 (s, 2H), 4.38 (d, J = 10.3 Hz, 2H), 4.25 (s, 2H), 4.21 (d, |
| | J = 5.4 Hz, 1H), 4.16 (d, J = 6.6 Hz, 2H), 3.90 (s, 1H), 3.81 (s, 2H), 3.57 (s, 3H), 2.99 (d, J = 7.2 Hz, 2H), |
| | 2.83 (d, J = 8.5 Hz, 1H), 2.73 (d, J = 10.5 Hz, 2H), 1.20 (t, J = 7.5 Hz, 3H). |
| 124 | 10.34 (s, 1H), 8.87 (t, J = 5.4 Hz, 1H), 8.07 (d, J = 8.5 Hz, 1H), 7.97 (s, 1H), 7.76-7.69 (m, 1H), 7.56- |
| | 7.49 (m, 1H), 7.17 (t, J = 8.0 Hz, 2H), 6.84 (s, 1H), 5.33(s, 2H), 4.40 (d, J = 12.3 Hz, 1H), 4.24(dd, J = 26.0, |
| | 8.3 Hz, 4H), 3.89 (s, 1H), 3.81 (t, J = 5.4 Hz, 2H), 3.59-3.33 (m, 4H), 3.24(s, 2H), 3.00(d, J = 7.4 Hz, |
| | 2H), 2.77 (d, J = 25.1 Hz, 2H), 1.20(t, J = 7.5 Hz, 3H). |
| 125 | 10.36 (s, 1H), 8.87 (t, J = 5.4 Hz, 1H), 8.06 (d, J = 8.6 Hz, 1H), 7.97 (s, 1H), 7.72 (dd, J = 8.9, 1.7 Hz, |
| | 1H), 7.51 (d, J = 1.8 Hz, 1H), 7.49 (s, 1H), 7.43 (dd, J = 9.2, 6.7 Hz, 1H), 6.83 (s, 1H), 5.32 (s, 2H), 4.40 |
| | (d, J = 13.7 Hz, 1H), 4.26 (s, 3H), 4.17 (dd, J = 17.4, 5.5 Hz, 1H), 3.93 (d, J = 12.1 Hz, 1H), 3.80 (t, |
| | J = 5.4 Hz, 2H), 3.53 (t, J = 11.6 Hz, 1H), 3.45-3.37 (m, 3H), 3.27-3.19 (m, 1H), 2.99 (dd, J = 14.7, 7.4 |
| | Hz, 2H), 2.81 (d, J = 12.9 Hz, 1H), 2.72 (d, J = 10.6 Hz, 2H), 1.20 (t, J = 7.5 Hz, 3H). |
| 126 | 10.36 (s, 1H), 8.70 (t, J = 5.8 Hz, 1H), 8.06 (d, J = 8.5 Hz, 1H), 7.97 (s, 1H), 7.73 (d, J = 8.5 Hz, 1H), |
| | 7.55 (s, 3H), 6.83 (s, 1H), 5.32 (s, 2H), 4.40 (d, J = 12.1 Hz, 1H), 4.26 (s, 2H), 4.19 (dd, J = 17.0, 5.7 Hz, |
| | 2H), 3.92 (d, J = 13.3 Hz, 1H), 3.80 (t, J = 5.4 Hz, 2H), 3.52 (t, J = 11.7 Hz, 1H), 3.41 (s, 1H), 3.25 (s, |
| | 2H), 3.00 (d, J = 7.3 Hz, 2H), 2.76 (dd, J = 28.9, 13.0 Hz, 4H), 1.21 (d, J = 7.2 Hz, 3H). |
| 127 | 10.35 (s, 1H), 8.62 (t, J = 5.7 Hz, 1H), 8.06 (d, J = 8.5 Hz, 1H), 7.97 (d, J = 1.5 Hz, 1H), 7.75-7.68 (m, |
| | 2H), 7.53 (d, J = 1.0 Hz, 2H), 6.83 (s, 1H), 5.33 (s, 2H), 4.40 (d, J = 12.1 Hz, 1H), 4.26 (d, J = 2.4 Hz, |
| | 2H), 4.19 (dd, J = 15.9, 5.7 Hz, 2H), 3.92 (d, J = 12.3 Hz, 1H), 3.80 (t, J = 5.5 Hz, 2H), 3.57-3.32 (m, |
| | 4H), 3.25 (s, 2H), 3.00 (d, J = 7.3 Hz, 2H), 2.77 (d, J = 26.5 Hz, 2H), 1.20 (t, J = 7.5 Hz, 3H). |
| 128 | 10.37 (s, 1H), 8.55 (t, J = 5.3 Hz, 1H), 8.06 (d, J = 8.6 Hz, 1H), 7.97 (s, 1H), 7.73 (d, J = 9.5 Hz, 2H), |
| | 6.84 (s, 1H), 5.33 (s, 2H), 4.38 (d, J = 12.0 Hz, 1H), 4.24 (d, J = 12.1 Hz, 3H), 4.17 (d, J = 5.5 Hz, 1H), |
| | 3.90 (d, J = 12.2 Hz, 1H), 3.81 (t, J = 5.2 Hz, 2H), 3.55-3.46 (m, 2H), 3.23 (d, J = 11.1 Hz, 2H), 2.99 |
| | (d, J = 7.1 Hz, 2H), 2.83 (d, J = 11.2 Hz, 2H), 2.73 (d, J = 10.8 Hz, 2H), 2.53 (s, 3H), 1.20 (t, J = 7.4 Hz, |
| | 3H). |
| 129 | 10.37 (s, 1H), 9.22 (d, J = 1.6 Hz, 1H), 8.42 (s, 1H), 8.36 (d, J = 1.6 Hz, 1H), 8.07 (d, J = 8.6 Hz, 1H), |
| | 7.97 (s, 1H), 7.72 (d, J = 8.9 Hz, 1H), 6.84 (s, 1H), 5.32 (s, 2H), 4.40 (d, J = 11.6 Hz, 1H), 4.25 (s, 3H), |
| | 4.20 (d, J = 4.8 Hz, 1H), 3.91 (d, J = 12.0 Hz, 1H), 3.80 (t, J = 5.0 Hz, 2H), 3.51 (t, J = 11.1 Hz, 1H), |
| | 3.41 (d, J = 11.1 Hz, 1H), 3.23 (d, J = 12.4 Hz, 2H), 3.00 (d, J = 6.7 Hz, 2H), 2.82 (t, J = 10.1 Hz, 2H), |
| | 2.73 (d, J = 10.4 Hz, 2H), 1.20 (t, J = 7.4 Hz, 3H). |
| 130 | 10.36 (s, 1H), 8.91 (t, J = 5.8 Hz, 1H), 8.06 (d, J = 8.5 Hz, 1H), 7.97 (d, J = 1.4 Hz, 1H), 7.72 (dd, |
| | J = 8.7, 1.6 Hz, 1H), 6.97 (s, 1H), 6.83 (s, 1H), 5.32 (s, 2H), 4.37 (d, J = 12.3 Hz, 1H), 4.25 (d, J = 2.3 Hz, |
| | 2H), 4.18 (dd, J = 12.6, 5.7 Hz, 2H), 3.92 (d, J = 12.5 Hz, 1H), 3.80 (t, J = 5.4 Hz, 2H), 3.51 (t, J = 10.7 |
| | Hz, 1H), 3.37 (s, 1H), 3.23 (d, J = 11.5 Hz, 2H), 2.99 (d, J = 7.6 Hz, 2H), 2.85-2.68 (m, 4H), 2.31(s, 3H), |
| | 1.19 (t, J = 7.5 Hz, 3H). |
| 131 | 10.35 (s, 1H), 8.76 (t, J = 5.7 Hz, 1H), 8.26 (s, 1H), 8.06 (d, J = 8.6 Hz, 1H) 7.97 (s, 1H), 7.72 (d, J = 8.6 |
| | Hz, 1H), 6.83 (s, 1H), 5.32 (s, 2H), 4.38 (d, J = 12.0 Hz, 1H), 4.26 (s, 2H), 4.17 (t, J = 5.3 Hz, 2H), 3.93 |
| | (d, J = 12.3 Hz, 1H), 3.80 (t, J = 5.3 Hz, 2H), 3.50 (t, J = 11.8 Hz, 1H), 3.39 (d, J = 9.4 Hz, 1H), 3.23 (d, |
| | J = 10.1 Hz, 2H), 2.99 (d, J = 7.2 Hz, 2H), 2.75 (dd, J = 24.3, 11.5 Hz, 4H), 2.68 (s, 3H), 1.19 (t, J = 7.5 |
| | Hz, 3H). |
| 132 | 10.38 (s, 1H), 8.78 (t, J = 5.6 Hz, 1H), 8.07 (d, J = 8.6 Hz, 1H), 7.98 (s, 1H), 7.96-7.90 (m, 1H), 7.79 |
| | (s, 1H), 7.73 (d, J = 7.0 Hz, 1H), 7.60 (dd, J = 10.4, 8.4 Hz, 1H), 6.84 (s, 1H), 5.33 (s, 2H), 4.38 (d, |
| | J = 12.3 Hz, 1H), 4.23 (dd, J = 20.4, 2.9 Hz, 4H), 3.96 (d, J = 12.7 Hz, 1H), 3.81 (t, J = 5.5 Hz, 2H), 3.51 (t, |
| | J = 10.2 Hz, 1H), 3.39 (d, J = 11.6 Hz, 1H), 3.24 (d, J = 11.7 Hz, 2H), 3.00 (d, J = 7.5 Hz, 2H), 2.73 (dd, |
| | J = 31.4, 20.1 Hz, 4H), 1.20 (t, J = 7.5 Hz, 3H). |

### Example 133: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoropicolinamide

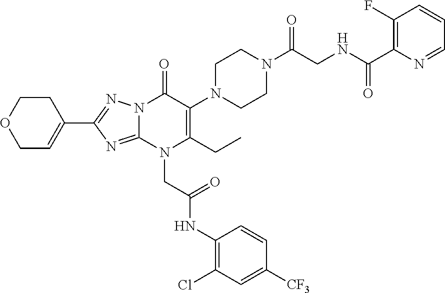 [SMILES: CCc1c(N2CCN(C(=O)CNC(=O)c3ncccc3F)CC2)c(=O)n2nc(C3=CCOCC3)nc2n1CC(=O)Nc1ccc(C(F)(F)F)cc1Cl]

To a mixture of 3-fluoropicolinic acid (14 mg, 0.1 mmol), N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (31 mg, 0.05 mmol, Example 38) and pyridine (40 mg, 0.5 mmol) in DCM (2 mL) was added POCl₃ (45 mg, 0.3 mmol), and then stirred at r.t. for 2 h. The mixture was diluted with MeOH (3 mL) and concentrated under reduced pressure. The residue was purified by flash chromatography on a C18 column eluting with MeCN/H₂O (45-65% with 0.1% NH₄HCO₃) to afford the title compound (21.1 mg) as a white solid. LCMS calc. for C₃₃H₃₁ClF₄N₉O₅ [M−H]⁻: m/z=744.2; Found: 744.3.

### Example 134: N-(2-Chloro-4-(trifluoromethyl)phenyl)-2-(2-(3,6-dihydro-2H-pyran-4-yl)-5-ethyl-6-(4-(methylglycyl)piperazin-1-yl)-7-oxo-[1,2,4]triazolo[1,5-a]pyrimidin-4(7H)-yl)acetamide

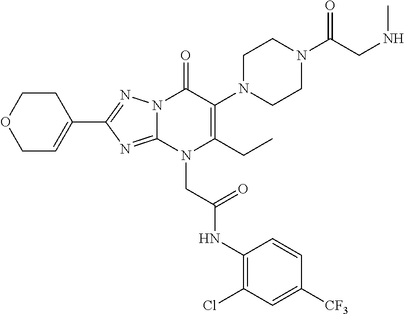 [SMILES: CCc1c(N2CCN(C(=O)CNC)CC2)c(=O)n2nc(C3=CCOCC3)nc2n1CC(=O)Nc1ccc(C(F)(F)F)cc1Cl]

### Step 1: tert-butyl (2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)(methyl)carbamate

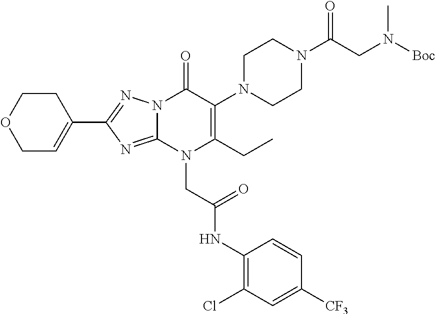 [SMILES: CCc1c(N2CCN(C(=O)CN(C)C(=O)OC(C)(C)C)CC2)c(=O)n2nc(C3=CCOCC3)nc2n1CC(=O)Nc1ccc(C(F)(F)F)cc1Cl]

A mixture of INT B1 (60 mg, 0.11 mmol), N-(tert-butoxycarbonyl)-N-methylglycine (22.1 mg, 0.12 mmol), NMP (13.9 mg, 0.14 mmol), EDCI (27 mg, 0.14 mmol) in DCM (2 mL) was stirred at r.t for 2 h. The reaction mixture was concentrated under reduced pressure. The residue was purified by Prep-HPLC on a C18 column eluting with MeCN/H₂O (55-85%, with 0.1% NH₄HCO₃) to afford the title compound (40 mg, 51% yield) as a white solid. LCMS calc. for C₃₃H₄₁ClF₃N₈₀₆ [M+H]⁺: m/z=737.3; Found: [M+H−100]⁺: m/z=637.0.

### Step 2: N-(2-chloro-4-(trifluoromethyl)phenyl)-2-(2-(3,6-dihydro-2H-pyran-4-yl)-5-ethyl-6-(4-(methylglycyl)piperazin-1-yl)-7-oxo-[1,2,4]triazolo[1,5-a]pyrimidin-4(7H)-yl)acetamide

A mixture of tert-butyl (2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)(methyl)carbamate (40 mg, 0.05 mmol) in 4 M HCl/dioxane (1 mL) was stirred at r.t for 2 h. The mixture was then concentrated under reduced pressure to afford the title compound (33 mg, 95% yield) as a white solid. LCMS calc. for C₂₈H₃₃ClF₃N₈O₄ [M+H]⁺: m/z=637.2; Found: 637.3.

### Example 135: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-N-methylbenzamide

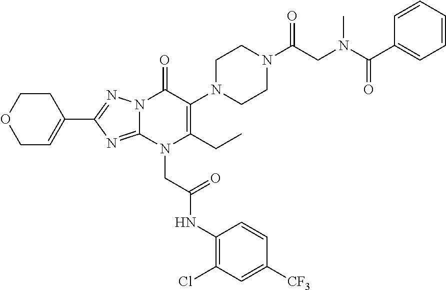 [SMILES: CCc1c(N2CCN(C(=O)CN(C)C(=O)c3ccccc3)CC2)c(=O)n2nc(C3=CCOCC3)nc2n1CC(=O)Nc1ccc(C(F)(F)F)cc1Cl]

A mixture of N-(2-chloro-4-(trifluoromethyl)phenyl)-2-(2-(3,6-dihydro-2H-pyran-4-yl)-5-ethyl-6-(4-(methylglycyl)piperazin-1-yl)-7-oxo-[1,2,4]triazolo[1,5-a]pyrimidin-4(7H)-yl)acetamide (28 mg, 44.0 μmol, Example 134), benzoyl chloride (6.8 mg, 48 μmol), TEA (17.8 mg, 176 μmol) in DCM (1 mL) was stirred at r.t for 2 h. The reaction mixture was concentrated and the residue was purified by Prep-HPLC on a C18 column eluting with MeCN/H₂O (55-75%, with 0.1% NH₄HCO₃) to give the title product (9.8 mg, 30% yield) as a white solid. ¹H NMR (400 MHZ, DMSO-d₆)₈ ppm 10.36 (s, 1H), 8.05 (t, J=7.3 Hz, 1H), 7.97 (s, 1H), 7.72 (d, J=8.5 Hz, 1H), 7.45 (d, J=9.8 Hz, 4H), 7.32 (s, 1H), 6.83 (s, 1H), 5.32 (d, J=9.5 Hz, 2H), 4.52-4.33 (m, 2H), 4.25 (d, J=2.4 Hz, 3H), 3.98 (dd, J=64.6, 14.2 Hz, 1H), 3.81 (t, J=5.4 Hz, 2H), 3.55 (dd, J=20.3, 12.1 Hz, 1H), 3.44-3.34 (m, 2H), 3.24 (d, J=11.3 Hz, 2H), 3.00 (d, J=11.5 Hz, 3H), 2.92 (s, 2H), 2.74 (dd, J=35.5, 23.6 Hz, 3H), 1.23-1.14 (m, 3H). LCMS calc. for C₃₅H₃₇ClF₃N₈O₅ [M+H]⁺: m/z=741.2; Found: 741.2 Example 136: N-(1-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-1-oxopropan-2-yl)benzamide

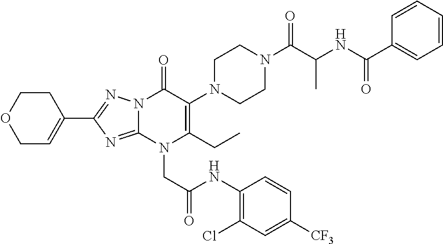 [SMILES: CCc1c(N2CCN(C(=O)C(C)NC(=O)c3ccccc3)CC2)c(=O)n2nc(C3=CCOCC3)nc2n1CC(=O)Nc1ccc(C(F)(F)F)cc1Cl]

### Step 1: tert-butyl (1-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-1-oxopropan-2-yl)carbamate

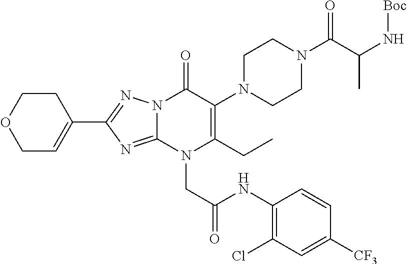 [SMILES: CCc1c(N2CCN(C(=O)C(C)NC(=O)OC(C)(C)C)CC2)c(=O)n2nc(C3=CCOCC3)nc2n1CC(=O)Nc1ccc(C(F)(F)F)cc1Cl]

To a mixture of (tert-butoxycarbonyl) alanine (20 mg, 0.11 mmol), HOBt (14.3 mg, 0.11 mmol), NMP (10.5 mg, 0.11 mmol) and EDCI (20.3 mg, 0.11 mmol) in DCM (5.0 mL) was added INT B1 (50 mg, 0.09 mmol). The mixture was stirred at r.t for 2 h. and concentrated. The residue was purified by Prep-HPLC on a C18 column eluting with MeOH/H₂O (55-80%, with 0.1% NH₄HCO₃) to afford the title compound (60 mg, 70% yield) as a yellow solid. LCMS calc. for C₃₃H₄₁ClF₃N₈O₆ [M+H]⁺: m/z=737.3; Found: [M+H−100]⁺: 637.2.

### Step 2: 2-(6-(4-alanylpiperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

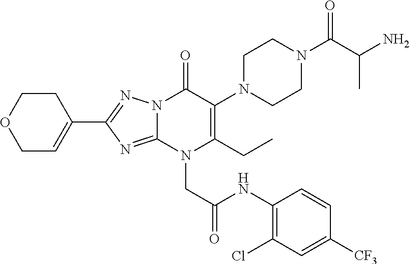 [SMILES: CCc1c(N2CCN(C(=O)C(C)N)CC2)c(=O)n2nc(C3=CCOCC3)nc2n1CC(=O)Nc1ccc(C(F)(F)F)cc1Cl]

A solution of tert-butyl (1-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl) piperazin-1-yl)-1-oxopropan-2-yl)carbamate (64 mg, 0.09 mmol) in 4 M HCl/dioxane (3 mL) was stirred at r.t. for 2 h. and concentrated under reduced pressure to afford the title compound (64 mg) as a yellow solid. ¹H NMR (400 MHZ, DMSO-d₆) δ ppm 10.41 (s, 1H), 8.19 (s, 2H), 8.05 (d, J=6.9 Hz, 1H), 7.98 (s, 1H), 7.72 (d, J=8.5 Hz, 1H), 6.83 (s, 1H), 5.36 (d, J=18.5 Hz, 2H), 4.46 (dt, J=25.3, 9.1 Hz, 2H), 4.26 (d, J=2.4 Hz, 2H), 3.91 (d, J=11.9 Hz, 1H), 3.81 (t, J=6.0 Hz, 2H), 3.57-3.43 (m, 2H), 3.39 (s, 2H), 3.26 (d, J=12.2 Hz, 2H), 3.06-2.94 (m, 2H), 2.76 (d, J=11.9 Hz, 2H), 1.35 (dd, J=31.7, 6.8 Hz, 3H), 1.19 (t, J=7.5 Hz, 3H). LCMS calc. for C₂₈H₃₃ClF₃N₈O₄ [M+H]⁺: m/z=637.2; Found: 637.2.

### Step 3: N-(1-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-1-oxopropan-2-yl)benzamide

To a solution of 2-(6-(4-alanylpiperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide (20 mg, 0.03 mmol) and TEA (19.09 mg, 0.12 mmol) in DCM (2 mL) was added benzoyl chloride (4.41 mg, 0.03 mmol) at 0° C. The reaction mixture was stirred from 0° C. to r.t. for 2 h. and then concentrated under reduced pressure. The residue was purified by Prep-HPLC on a C18 column eluting with MeOH/H₂O (40-75%, with 0.1% NH₄HCO₃) to afford the title compound (9.4 mg, 40% yield) as a white solid. ¹H NMR (400 MHZ, DMSO-d₆) δ ppm 10.40 (s, 1H), 8.66 (t, J=8.3 Hz, 1H), 8.05 (d, J=8.6 Hz, 1H), 7.97 (d, J=1.4 Hz, 1H), 7.91 (s, 2H), 7.72 (dd, J=8.7, 1.6 Hz, 1H), 7.54 (t, J=7.3 Hz, 1H), 7.47 (t, J=7.5 Hz, 2H), 6.83 (s, 1H), 5.32 (s, 2H), 5.08-4.93 (m, 1H), 4.42 (d, J=11.2 Hz, 1H), 4.26 (s, 2H), 4.10-3.97 (m, 1H), 3.80 (t, J=5.2 Hz, 2H), 3.50 (d, J=11.9 Hz, 4H), 3.04-2.95 (m, 2H), 2.84-2.65 (m, 4H), 1.24 (s, 3H), 1.19 (t, J=7.5 Hz, 3H). LCMS calc. for C₃₅H₃₇ClF₃N₈O₅ [M+H]⁺: m/z=741.2; Found: 741.2.

### Example 137: N-(1-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-methyl-1-oxopropan-2-yl)benzamide

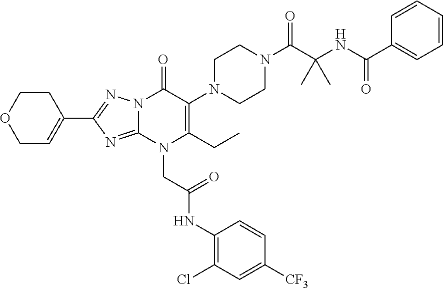 [SMILES: CCc1c(N2CCN(C(=O)C(C)(C)NC(=O)c3ccccc3)CC2)c(=O)n2nc(C3=CCOCC3)nc2n1CC(=O)Nc1ccc(C(F)(F)F)cc1Cl]

To a mixture of INT B1 (30 mg, 0.05 mmol), 2-benzamido-2-methylpropanoic acid (12 mg, 0.06 mmol), HOBt (13 mg, 0.06 mmol) and EDCI (8 mg, 0.07 mmol) in DMF (1 mL) was added DIEA (25 mg, 0.19 mmol) at 0° C. under N₂ atmosphere. The resulting mixture was stirred at r.t. for 3 h., and concentrated. The residue was purified by Prep-HPLC on a C18 column eluting with MeCN/H₂O (30-50%, with 0.1% NH₄HCO₃) to afford the title product (5 mg, 14% yield) as a white solid. ¹H NMR (400 MHZ, DMSO-d₆) δ ppm 10.33 (s, 1H), 8.56 (d, J=3.6 Hz, 1H), 8.03 (d, J=8.3 Hz, 1H), 7.95 (d, J=1.5 Hz, 1H), 7.87-7.83 (m, 2H), 7.73-7.68 (m, 1H), 7.55-7.50 (m, 1H), 7.46 (t, J=7.3 Hz, 2H), 6.82 (s, 1H), 5.28 (s, 2H), 4.45 (d, J=12.1 Hz, 2H), 4.25 (d, J=2.5 Hz, 2H), 3.80 (t, J=5.5 Hz, 2H), 3.37 (s, 6H), 2.97-2.89 (m, 2H), 2.62 (d, J=9.3 Hz, 2H), 1.49 (s, 6H), 1.13 (t, J=7.6 Hz, 3H). LCMS calc. for C₃₆H₃₉ClF₃N₈O₅ [M+H]⁺: m/z=755.3; Found: 755.2.

### Example 138: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl-1,1-d₂)-2-fluorobenzamide

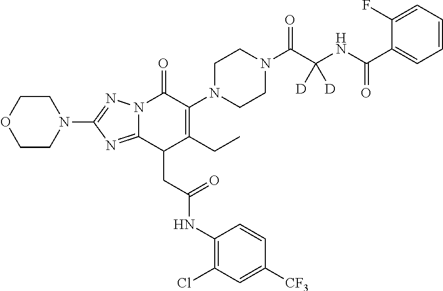 [SMILES: [2H]C([2H])(NC(=O)c1ccccc1F)C(=O)N1CCN(C2=C(CC)C(CC(=O)Nc3ccc(C(F)(F)F)cc3Cl)c3nc(N4CCOCC4)nn3C2=O)CC1]

A mixture of (2-fluorobenzoyl)glycine-2,2-d₂ (40 mg, 0.2 mmol), INT B4 (100 mg, 0.18 mmol), EDCI (54 mg, 0.3 mmol), HOBt (40 mg, 0.3 mmol) and TEA (101 mg, 1 mmol) in DMF (1 mL) was stirred at r.t. overnight. The mixture was concentrated under reduced pressure. The residue was purified by flash chromatography on a C18 column eluting with MeCN/H₂O (60-75% with 0.1% NH₄HCO₃) to afford the title compound (72 mg) as a white solid. LCMS calc. for C₃₃H₃₁D₂ClF₄N₉O₅ [M−H]⁻: m/z=748.2; Found: 748.1.

### Example 139: N-(2-(7-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-2,7-diazaspiro[4.4]nonan-2-yl)-2-oxoethyl)benzamide

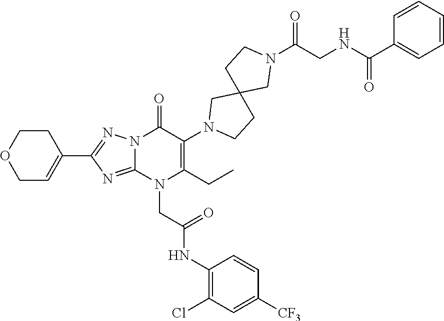 [SMILES: CCc1c(N2CCC3(CCN(C(=O)CNC(=O)c4ccccc4)C3)C2)c(=O)n2nc(C3=CCOCC3)nc2n1CC(=O)Nc1ccc(C(F)(F)F)cc1Cl]

A mixture of INT B8 (25 mg, 0.04 mmol), 2-benzamidoacetic acid (7 mg, 0.05 mmol), HATU (23 mg, 0.06 mmol) and DIEA (22 mg, 0.16 mmol) in DCM (2 mL) was stirred at r.t. overnight. The reaction was diluted with water (10 mL), extracted with DCM (5 mL×3). The combined organic layers were dried over anhydrous Na₂SO₄, filtered and concentrated under reduced pressure. The residue was purified by Prep-HPLC on a C18 column eluting with MeCN/H₂O (15-50%, with 0.1% FA) to afford the title compound (8.95 mg, 31.4% yield) as a white solid. ¹H NMR (400 MHZ, DMSO-d₆) δ ppm 10.37 (d, J=2.3 Hz, 1H), 8.62-8.53 (m, 1H), 8.09-8.03 (m, 1H), 7.96 (d, J=2.2 Hz, 1H), 7.87 (dt, J=8.5, 1.3 Hz, 2H), 7.75-7.69 (m, 1H), 7.57-7.51 (m, 1H), 7.50-7.44 (m, 2H), 6.83 (h, J=1.5 Hz, 1H), 5.31 (d, J=4.0 Hz, 2H), 4.25 (q, J=2.8 Hz, 2H), 4.05 (dd, J=7.1, 4.6 Hz, 2H), 3.80 (t, J=5.4 Hz, 2H), 3.68-3.46 (m, 3H), 3.45-3.34 (m, 2H), 3.24 (s, 2H), 3.09 (dd, J=7.3, 4.7 Hz, 3H), 2.96-2.87 (m, 2H), 2.16-1.83 (m, 4H), 1.22-1.17 (m, 3H). LCMS calc. for C₃₇H₃₉ClF₃N₈O₅ [M+H]⁺: m/z=767.3; Found: 767.0.

### Example 140: N-(2-(5-(4-(2-((2-Chloro-4-(trifluoromethyl)phenyl)amino)-2-oxoethyl)-2-(3,6-di hydro-2H-pyran-4-yl)-5-ethyl-7-oxo-4,7-dihydro-[1,2,4]triazolo[1,5-a]pyrimidin-6-yl) hexahydropyrrolo[3,4-c]pyrrol-2(1H)-yl)-2-oxoethyl)-2-fluorobenzamide

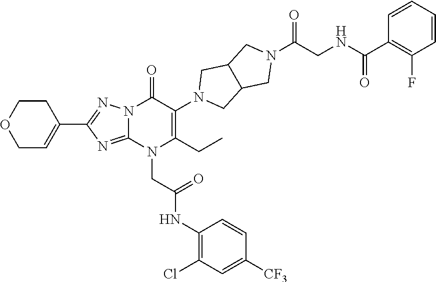 [SMILES: CCc1c(N2CC3CN(C(=O)CNC(=O)c4ccccc4F)CC3C2)c(=O)n2nc(C3=CCOCC3)nc2n1CC(=O)Nc1ccc(C(F)(F)F)cc1Cl]

A mixture of (2-fluorobenzoyl)glycine (20 mg, 0.10 mmol), HATU (29 mg, 0.08 mmol) and K₂CO₃ (21 mg, 0.15 mmol) in MeCN (2 mL) was stirred at r.t. for 30 min., and then INT B9 was added. The mixture was stirred at r.t. for 3 h., and filtered. The filtrate was concentrated under reduced pressure. The residue was purified by Prep-HPLC on a C18 column eluting with MeCN/H₂O (5-60%, with 0.1% FA) to afford the title product (11.8 mg, 30% yield) as a white solid. ¹H NMR (400 MHZ, DMSO-d₆) δ ppm 10.32 (s, 1H), 8.32-8.26 (m, 1H), 8.06 (d, J=8.8 Hz, 1H), 7.97-7.94 (m, 1H), 7.78-7.73 (m, 1H), 7.73-7.69 (m, 1H), 7.60-7.53 (m, 1H), 7.36-7.32 (m, 1H), 7.32-7.29 (m, 1H), 6.80-6.77 (m, 1H), 5.04 (s, 2H), 4.27-4.22 (m, 2H), 4.10 (d, J=5.2 Hz, 2H), 3.83-3.76 (m, 3H), 3.73-3.65 (m, 1H), 3.52 (dd, J=10.4, 4.4 Hz, 1H), 3.45-3.39 (m, 3H), 3.09-2.99 (m, 4H), 2.98-2.85 (m, 4H), 1.27 (t, J=7.6 Hz, 3H). LCMS calc. for C₃₆H₃₆ClF₄N₈O₅ [M+H]⁺: m/z=771.2; Found: 771.1.

### Example 141: N-(2-((1-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl) piperidin-4-yl)amino)-2-oxoethyl)benzamide

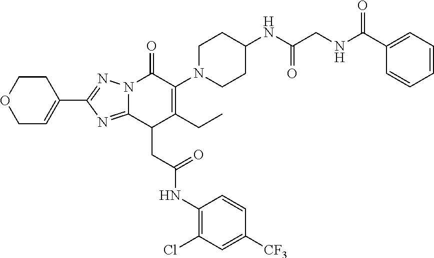 [SMILES: CCC1=C(N2CCC(NC(=O)CNC(=O)c3ccccc3)CC2)C(=O)n2nc(C3=CCOCC3)nc2C1CC(=O)Nc1ccc(C(F)(F)F)cc1Cl]

A mixture of INT B10 (15 mg, 0.03 mmol), benzoylglycine, HATU (12 mg, 0.03 mmol) and K₂CO₃ (9 mg, 0.06 mmol) in DMF (2 mL) was stirred at r.t. overnight. The mixture was filtered, and concentrated under reduced pressure. The residue was purified by Prep-HPLC on a C18 column eluting with MeCN/H₂O (30%-50%, with 0.1% FA) to afford the title compound (5 mg, 27% yield) as a white solid. ¹H NMR (400 MHZ, DMSO-d₆)₈ ppm 10.31 (s, 1H), 8.68 (s, 1H), 8.06 (d, J=8.6 Hz, 1H), 7.96 (d, J=1.3 Hz, 1H), 7.94-7.85 (m, 3H), 7.71 (dd, J=8.7, 1.6 Hz, 1H), 7.54 (t, J=7.2 Hz, 1H), 7.48 (t, J=7.3 Hz, 2H), 6.78 (s, 1H), 5.03 (s, 2H), 4.29-4.21 (m, 2H), 3.87 (d, J=5.3 Hz, 2H), 3.80 (t, J=5.4 Hz, 2H), 3.64 (s, 1H), 3.42 (s, 2H), 3.07 (q, J=7.4 Hz, 2H), 2.75 (s, 2H), 2.52 (d, J=1.9 Hz, 2H), 1.79 (d, J=9.7 Hz, 2H), 1.62-1.49 (m, 2H), 1.26 (t, J=7.5 Hz, 3H). LCMS calc. for C₃₅H₃₇ClF₃N₈O₅ [M+H]⁺: m/z=741.2; Found: 741.2.

### Example 142: N-(2-(4-(4-(2-((2-Chloro-4-(trifluoromethyl)phenyl)amino)-2-oxoethyl)-2-(3,6-dihydro-2H-pyran-4-yl)-5-hydroxy-7-oxo-4,7-dihydro-[1,2,4]triazolo[1,5-a]pyrimidin-6-yl)piperazin-1-yl)-2-oxoethyl)benzamide

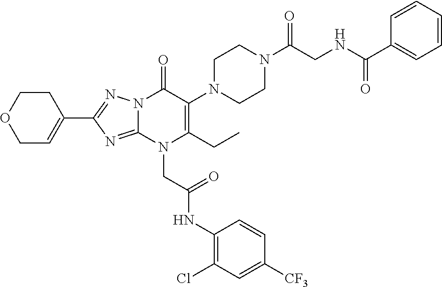 [SMILES: CCc1c(N2CCN(C(=O)CNC(=O)c3ccccc3)CC2)c(=O)n2nc(C3=CCOCC3)nc2n1CC(=O)Nc1ccc(C(F)(F)F)cc1Cl]

To a solution of INT B11 (30 mg, 0.05 mmol), benzoylglycine (12 mg, 0.07 mmol) and NMI (16 mg, 0.19 mmol) in MeCN (2 mL) was added TCFH (18 mg, 0.07 mmol) at 0° C. under N₂ atmosphere. The resulting mixture was stirred at r.t. for 3 h., and concentrated. The residue was purified by Prep-HPLC on a C18 column eluting with MeCN/H₂O (30-50%, with 0.1% NH₄HCO₃) to afford the title compound (9.02 mg, 23% yield) as a white solid. ¹H NMR (400 MHZ, DMSO-d₆) δ ppm 10.18 (s, 1H), 8.65 (t, J=5.7 Hz, 1H), 8.07 (d, J=8.5 Hz, 1H), 7.95 (s, 1H), 7.89 (d, J=7.1 Hz, 2H), 7.71 (d, J=7.3 Hz, 1H), 7.54 (d, J=7.2 Hz, 1H), 7.48 (t, J=7.3 Hz, 2H), 6.70 (s, 1H), 4.94 (s, 2H), 4.46 (d, J=12.6 Hz, 1H), 4.19 (d, J=38.1 Hz, 6H), 4.07 (d, J=25.4 Hz, 2H), 3.79 (t, J=5.2 Hz, 2H), 3.31-3.19 (m, 3H), 2.49-2.45 (m, 2H). LCMS calc. for C₃₂H₃₁ClF₃N₈₀₆ [M+H]⁺: m/z=715.2; Found: 715.2.

### Example 143: N-(2-(4-(4-(2-((2-Chloro-4-(trifluoromethyl)phenyl)(methyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzamide

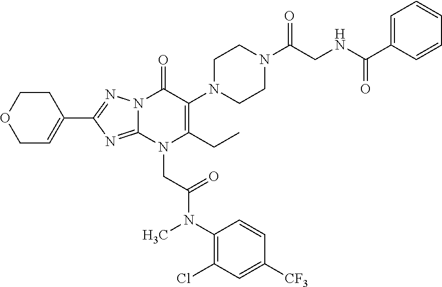 [SMILES: CCc1c(N2CCN(C(=O)CNC(=O)c3ccccc3)CC2)c(=O)n2nc(C3=CCOCC3)nc2n1CC(=O)N(C)c1ccc(C(F)(F)F)cc1Cl]

A mixture of INT B7 (20 mg, 0.04 mmol), benzoylglycine (12 mg, 0.07 mmol), TCFH (20 mg, 0.07 mmol) and NMI (13 mg, 0.14 mmol) in MeCN (1 mL) was stirred overnight at r.t. The reaction mixture was concentrated under reduced pressure. The residue was purified by Prep-HPLC on a C₁₈ column eluting with MeCN/H₂O (45-60%, with 0.1% FA) to afford the title compound (5 mg, 20% yield) as a white solid. ¹H NMR (400 MHZ, DMSO-d₆)₈ ppm 8.59 (t, J=5.7 Hz, 1H), 8.25 (d, J=1.6 Hz, 1H), 8.09 (d, J=8.2 Hz, 1H), 8.00 (d, J=2.0 Hz, 1H), 7.91-7.88 (m, 2H), 7.52 (dd, J=18.0, 7.4 Hz, 3H), 6.85 (s, 1H), 4.87-4.60 (m, 2H), 4.38 (d, J=12.4 Hz, 1H), 4.31-4.27 (m, 2H), 4.19 (d, J=5.7 Hz, 2H), 3.96 (d, J=13.2 Hz, 1H), 3.82 (t, J=5.4 Hz, 2H), 3.50 (s, 2H), 3.19 (s, 3H), 2.90 (s, 2H), 2.79 (t, J=11.3 Hz, 1H), 2.69 (d, J=11.1 Hz, 3H), 1.17 (dt, J=36.3, 7.5 Hz, 5H). LCMS calc. for C₃₅H₃₇ClF₃N₈O₅ [M+H]⁺: m/z=741.2; Found: 741.6.

### Example 144: N-(2-(4-(2-(3,6-Dihydro-2H-pyran-4-yl)-5-ethyl-4-(2-((5-fluoro-2-methyl-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl)-2-fluorobenzamide

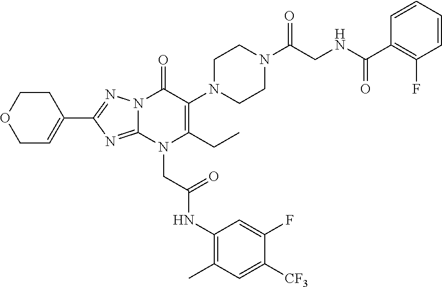 [SMILES: CCc1c(N2CCN(C(=O)CNC(=O)c3ccccc3F)CC2)c(=O)n2nc(C3=CCOCC3)nc2n1CC(=O)Nc1cc(F)c(C(F)(F)F)cc1C]

To a solution of INT D1 (35 mg, 0.06 mmol), 5-fluoro-2-methyl-4-(trifluoromethyl) aniline (14 mg, 0.07 mmol) and TEA (31 mg, 0.31 mmol) in DCM (2 mL) was added T₃P (472 mg, 50% wt in EtOAc, 0.72 mmol) at r.t. The mixture was stirred at r.t. for 1 h., and concentrated under reduced pressure. The residue was purified by Prep-HPLC on a C18 column eluting with MeCN/H₂O (5-50%, with 0.1% NH₄HCO₃) to afford the title compound (23.9 mg, 52% yield) as a white solid. ¹H NMR (400 MHZ, DMSO-d₆) δ ppm 10.08 (s, 1H), 8.33 (q, J=4.8 Hz, 1H), 7.83-7.72 (m, 2H), 7.68 (d, J=8.0 Hz, 1H), 7.61-7.54 (m, 1H), 7.37-7.29 (m, 2H), 6.82 (s, 1H), 5.30 (s, 2H), 4.41 (d, J=12.0 Hz, 1H), 4.31-4.13 (m, 4H), 3.92 (d, J=12.0 Hz, 1H), 3.81 (t, J=5.2 Hz, 2H), 3.57-3.46 (m, 1H), 3.45-3.36 (m, 1H), 3.30-3.20 (m, 1H), 3.05-2.94 (m, 2H), 2.87-2.77 (m, 1H), 2.77-2.69 (m, 2H), 2.54-2.51 (m, 2H), 2.36 (s, 3H), 1.20 (t, J=7.2 Hz, 3H). LCMS calc. for C₃₅H₃₆F₅N₈O₅ [M+H]⁺: m/z=743.3; Found: 743.5.

### Example 145: N-(2-(4-(2-(3,6-Dihydro-2H-pyran-4-yl)-5-ethyl-4-(2-((2-methyl-4-(pentafluoro-16-sulfanyl)phenyl)amino)-2-oxoethyl)-7-oxo-4,7-dihydro-[1,2,4]triazolo[1,5-a]pyrimidin-6-yl)piperazin-1-yl)-2-oxoethyl)-2-fluorobenzamide

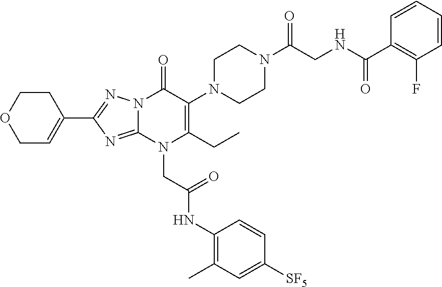

This compound was prepared by procedures analogous to those described for Example 144 Step 3 using INT D1 and 2-methyl-4-(pentafluoro-16-sulfanyl) aniline to afford the title compound as a white solid. ¹H NMR (400 MHZ, DMSO-d₆) δ ppm 10.05 (s, 1H), 8.32 (q, J=5.2 Hz, 1H), 7.82 (d, J=2.4 Hz, 1H), 7.78-7.70 (m, 3H), 7.61-7.54 (m, 1H), 7.36-7.29 (m, 2H), 6.83 (s, 1H), 5.26 (s, 2H), 4.40 (d, J=14.4 Hz, 1H), 4.30-4.17 (m, 4H), 3.91 (d, J=13.0 Hz, 1H), 3.81 (t, J=5.4 Hz, 2H), 3.52 (t, J=11.2 Hz, 1H), 3.40 (t, J=9.8 Hz, 1H), 3.29-3.20 (m, 1H), 3.01 (dd, J=14.6, 7.0 Hz, 2H), 2.84 (d, J=12.6 Hz, 1H), 2.73 (d, J=10.6 Hz, 2H), 2.54-2.51 (m, 2H), 2.38 (s, 3H), 1.20 (t, J=7.4 Hz, 3H). LCMS calc. for C₃₄H₃₇F₆N₈O₅S [M+H]⁺: m/z=783.2; Found: 783.2. Example 146: N-(3-Bromo-5-methylthiophen-2-yl)-2-(2-(3,6-dihydro-2H-pyran-4-yl)-5-ethyl-6-(4-(4-hydroxypyrrolo[1,2-b]pyridazine-3-carbonyl)piperazin-1-yl)-7-oxo-[1,2,4]triazolo[1,5-a]pyrimidin-4(7H)-yl)acetamide

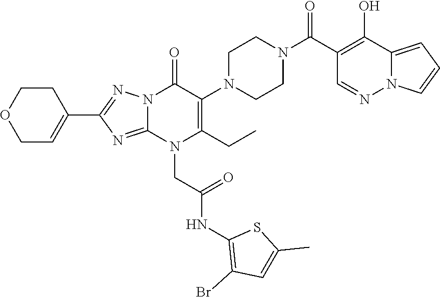 [SMILES: CCc1c(N2CCN(C(=O)c3cnn4cccc4c3O)CC2)c(=O)n2nc(C3=CCOCC3)nc2n1CC(=O)Nc1sc(C)cc1Br]

This compound was prepared by procedures analogous to those described for method A using INT C31 and INT B6 to afford the title product as a white solid. ¹H NMR (400 MHZ, DMSO-d₆) δ ppm 11.92 (s, 1H), 10.88 (s, 1H), 7.96 (s, 1H), 7.81 (dd, J=2.6, 1.6 Hz, 1H), 6.94 (dd, J=4.4, 1.6 Hz, 1H), 6.81 (s, 1H), 6.77 (dd, J=4.3, 2.7 Hz, 1H), 6.68 (d, J=1.2 Hz, 1H), 5.31 (s, 2H), 4.25 (d, J=2.4 Hz, 2H), 4.09 (d, J=11.3 Hz, 2H), 3.80 (t, J=5.4 Hz, 3H), 3.50 (d, J=2.5 Hz, 3H), 3.14 (t, J=11.1 Hz, 2H), 2.95 (dd, J=14.3, 6.8 Hz, 2H), 2.74 (d, J=10.8 Hz, 2H), 2.33 (s, 3H), 1.16 (t, J=7.4 Hz, 3H). LCMS calc. for C₃₁H₃₃BrN₉O₅S [M+H]⁺: m/z=722.1; Found: 722.0.

### Example 147: N-(2-(4-(2-(3,6-Dihydro-2H-pyran-4-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl)-2-fluorobenzamide

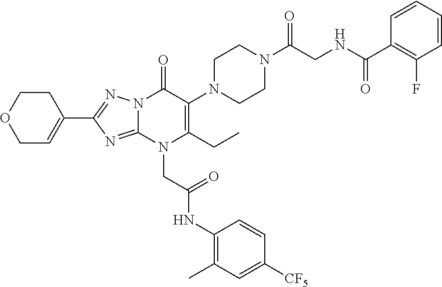

Method D: To a solution of INT D1 (50 mg, 0.09 mmol) in CH₃CN (1 mL) was added 2-methyl-4-(trifluoromethyl) aniline (31 mg, 0.18 mmol), pyridine (21 mg, 0.26 mmol) and POCl₃ (20 mg, 0.13 mmol). The mixture was stirred at 0° C. for 30 min. And the reaction mixture was quenched with water (1 mL), extracted with DCM (10 mL×3). The combined organic layers were washed with brine (5 mL), dried over anhydrous Na₂SO₄, filtered and concentrated under reduced pressure. The residue was purified by Prep-HPLC on a C18 column eluting with MeCN/H₂O (60%) to afford the title compound (5.2 mg, 8% yield) as a brown solid. LCMS calc. for C₃₅H₃₇F₄N₈O₅ [M+H]⁺: m/z=725.3; Found: 725.4.

The following compounds listed in Table 5 were prepared by using an appropriate amine and INT D1 as the methods analogous to those described for Method D.

TABLE 5
Preparations of Examples (Ex)

| | | | | LCMS |
|---|---|---|---|---|
| | | | | Cacl./ |
| Ex | Amine | Structure | Name | Found |
| 148 | 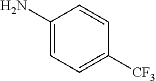 [SMILES: Nc1ccc(C(F)(F)F)cc1] | 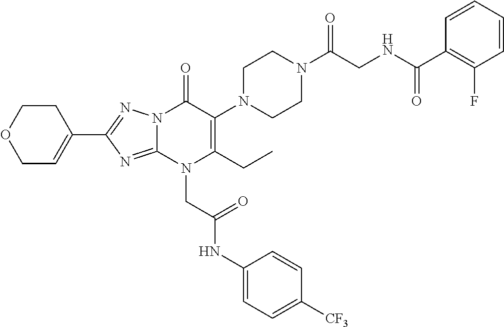 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1] | N-(2-(4-(2-(3,6-Dihydro-2H-pyran- 4-yl)-5-ethyl-7-oxo-4-(2-oxo-2-((4- (trifluoromethyl)phenyl)amino)ethyl)- 4,7-dihydro-[1,2,4]triazolo[1,5- a]pyrimidin-6-yl)piperazin-1-yl)-2- oxoethyl)-2-fluorobenzamide | [M + H]⁺: 711.3/ 711.5 |
| 149 | 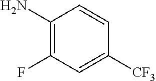 [SMILES: Nc1ccc(C(F)(F)F)cc1F] | 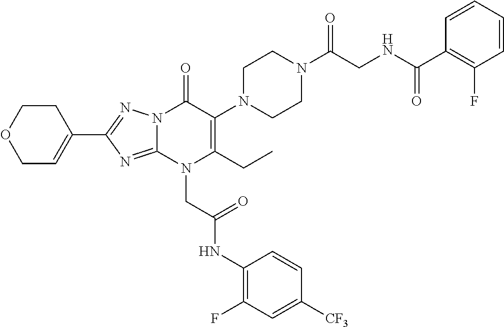 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-2- fluorobenzamide | [M + H]⁺: 729.3/ 729.4 |
| 150 | 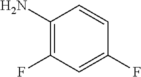 | 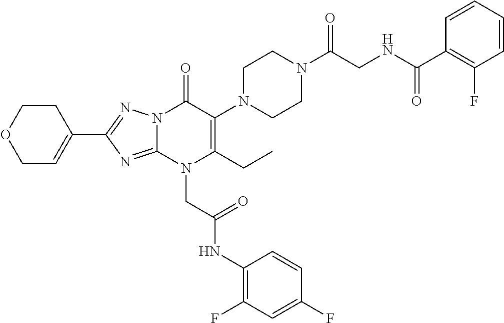 [SMILES: CCc1c(N2CCN(C(=O)CNC(=O)c3ccccc3F)CC2)c(=O)n2nc(C3=CCOCC3)nc2n1CC(=O)Nc1ccc(F)cc1F] | N-(2-(4-(4-(2-((2,4- Difluorophenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-fluorobenzamide | [M + H]⁺: 679.3/ 679.2 |
| 151 | 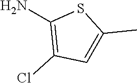 | 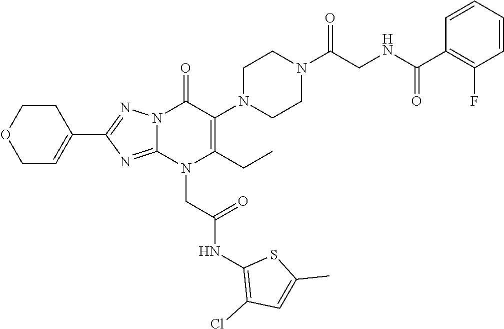 [SMILES: CCc1c(N2CCN(C(=O)CNC(=O)c3ccccc3F)CC2)c(=O)n2nc(C3=CCOCC3)nc2n1CC(=O)Nc1sc(C)cc1Cl] | N-(2-(4-(4-(2-((3-chloro-5- methylthiophen-2-yl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-fluorobenzamide | [M + H]⁺: 697.2/ 697.2 |
| 152 | 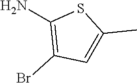 | 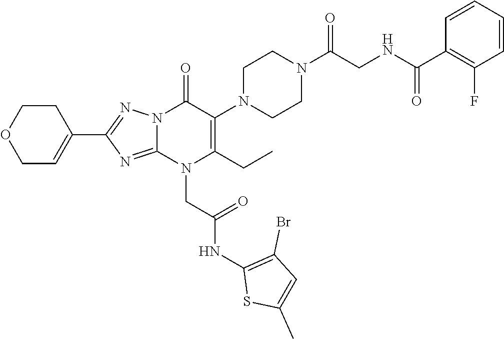 [SMILES: CCc1c(N2CCN(C(=O)CNC(=O)c3ccccc3F)CC2)c(=O)n2nc(C3=CCOCC3)nc2n1CC(=O)Nc1sc(C)cc1Br] | N-(2-(4-(4-(2-((3-bromo-5- methylthiophen-2-yl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-fluorobenzamide | [M + H]⁺: 741.2/ 741.2 |
| 153 | 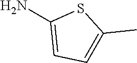 | 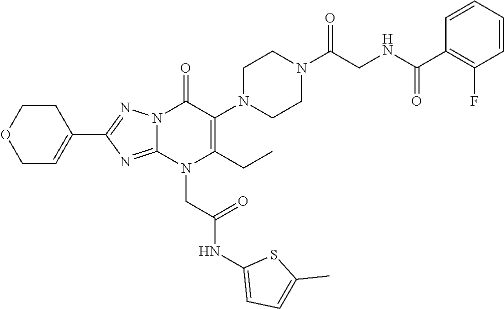 [SMILES: CCc1c(N2CCN(C(=O)CNC(=O)c3ccccc3F)CC2)c(=O)n2nc(C3=CCOCC3)nc2n1CC(=O)Nc1ccc(C)s1] | N-(2-(4-(2-(3,6-Dihydro-2H-pyran- 4-yl)-5-ethyl-4-(2-((5- methylthiophen-2-yl)amino)-2- oxoethyl)-7-oxo-4,7-dihydro- [1,2,4]triazolo[1,5-a]pyrimidin-6- yl)piperazin-1-yl)-2-oxoethyl)-2- fluorobenzamide | [M + H]⁺: 663.2/ 663.2 |
| 154 | 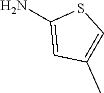 | 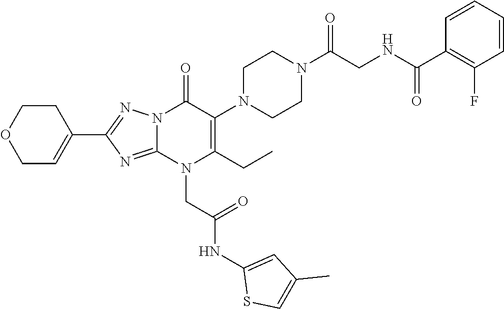 [SMILES: CCc1c(N2CCN(C(=O)CNC(=O)c3ccccc3F)CC2)c(=O)n2nc(C3=CCOCC3)nc2n1CC(=O)Nc1cc(C)cs1] | N-(2-(4-(2-(3,6-Dihydro-2H-pyran- 4-yl)-5-ethyl-4-(2-((4- methylthiophen-2-yl)amino)-2- oxoethyl)-7-oxo-4,7-dihydro- [1,2,4]triazolo[1,5-a]pyrimidin-6- yl)piperazin-1-yl)-2-oxoethyl)-2- fluorobenzamide | [M + H]⁺: 663.2/ 663.1 |
| 155 | 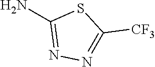 | 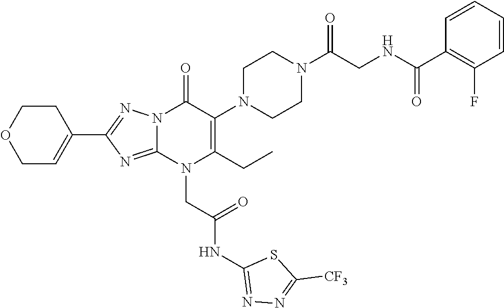 [SMILES: CCc1c(N2CCN(C(=O)CNC(=O)c3ccccc3F)CC2)c(=O)n2nc(C3=CCOCC3)nc2n1CC(=O)Nc1nnc(C(F)(F)F)s1] | N-(2-(4-(2-(3,6-Dihydro-2H-pyran- 4-yl)-5-ethyl-7-oxo-4-(2-oxo-2-((5- (trifluoromethyl)-1,3,4-thiadiazol- 2-yl)amino)ethyl)-4,7-dihydro- [1,2,4]triazolo[1,5-a]pyrimidin-6- yl)piperazin-1-yl)-2-oxoethyl)-2- fluorobenzamide | [M + H]⁺: 719.2/ 719.2 |
| 156 | 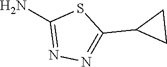 | 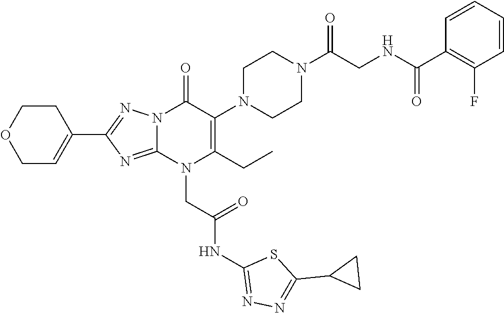 [SMILES: CCc1c(N2CCN(C(=O)CNC(=O)c3ccccc3F)CC2)c(=O)n2nc(C3=CCOCC3)nc2n1CC(=O)Nc1nnc(C2CC2)s1] | N-(2-(4-(4-(2-((5-Cyclopropyl- 1,3,4-thiadiazol-2-yl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-fluorobenzamide | [M + H]⁺: 691.2/ 691.2 |
| 157 | 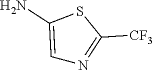 | 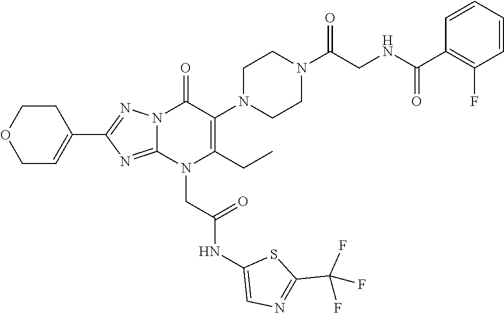 [SMILES: CCc1c(N2CCN(C(=O)CNC(=O)c3ccccc3F)CC2)c(=O)n2nc(C3=CCOCC3)nc2n1CC(=O)Nc1cnc(C(F)(F)F)s1] | N-(2-(4-(2-(3,6-Dihydro-2H-pyran- 4-yl)-5-ethyl-7-oxo-4-(2-oxo-2-((2- (trifluoromethyl)thiazol-5- yl)amino)ethyl)-4,7-dihydro- [1,2,4]triazolo[1,5-a]pyrimidin-6- yl)piperazin-1-yl)-2-oxoethyl)-2- fluorobenzamide | [M + H]⁺: 718.2/ 718.2 |
| 158 | 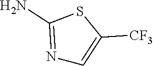 | 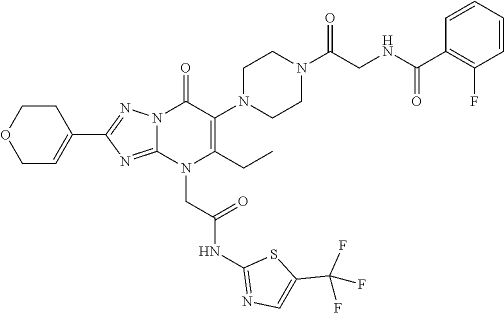 [SMILES: CCc1c(N2CCN(C(=O)CNC(=O)c3ccccc3F)CC2)c(=O)n2nc(C3=CCOCC3)nc2n1CC(=O)Nc1ncc(C(F)(F)F)s1] | N-(2-(4-(2-(3,6-Dihydro-2H-pyran- 4-yl)-5-ethyl-7-oxo-4-(2-oxo-2-((5- (trifluoromethyl)thiazol-2- yl)amino)ethyl)-4,7-dihydro- [1,2,4]triazolo[1,5-a]pyrimidin-6- yl)piperazin-1-yl)-2-oxoethyl)-2- fluorobenzamide | [M + H]⁺: 718.2/ 718.2 |
| 159 | 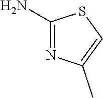 | 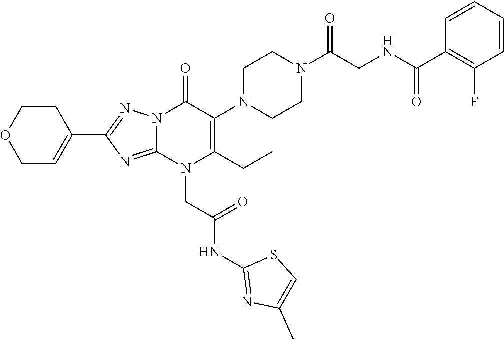 [SMILES: CCc1c(N2CCN(C(=O)CNC(=O)c3ccccc3F)CC2)c(=O)n2nc(C3=CCOCC3)nc2n1CC(=O)Nc1nc(C)cs1] | N-(2-(4-(2-(3,6-Dihydro-2H-pyran- 4-yl)-5-ethyl-4-(2-((4- methylthiazol-2-yl)amino)-2- oxoethyl)-7-oxo-4,7-dihydro- [1,2,4]triazolo[1,5-a]pyrimidin-6- yl)piperazin-1-yl)-2-oxoethyl)-2- fluorobenzamide | [M + H]⁺: 664.2/ 664.0 |
| 160 | 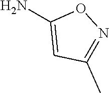 | 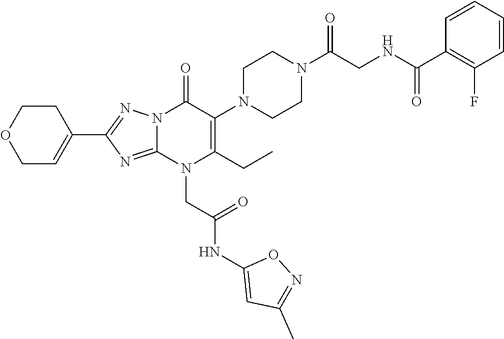 [SMILES: CCc1c(N2CCN(C(=O)CNC(=O)c3ccccc3F)CC2)c(=O)n2nc(C3=CCOCC3)nc2n1CC(=O)Nc1cc(C)no1] | N-(2-(4-(2-(3,6-Dihydro-2H-pyran- 4-yl)-5-ethyl-4-(2-((3- methylisoxazol-5-yl)amino)-2- oxoethyl)-7-oxo-4,7-dihydro- [1,2,4]triazolo[1,5-a]pyrimidin-6- yl)piperazin-1-yl)-2-oxoethyl)-2- fluorobenzamide | [M + H]⁺: 648.3/ 648.2 |
| 161 | 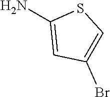 | 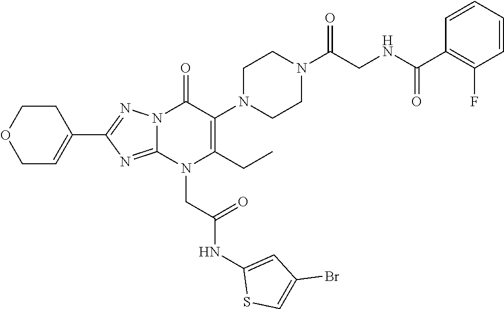 [SMILES: CCc1c(N2CCN(C(=O)CNC(=O)c3ccccc3F)CC2)c(=O)n2nc(C3=CCOCC3)nc2n1CC(=O)Nc1cc(Br)cs1] | N-(2-(4-(4-(2-((4-Bromothiophen- 2-yl)amino)-2-oxoethyl)-2-(3,6- dihydro-2H-pyran-4-yl)-5-ethyl-7- oxo-4,7-dihydro- [1,2,4]triazolo[1,5-a]pyrimidin-6- yl)piperazin-1-yl)-2-oxoethyl)-2- fluorobenzamide | [M + H]⁺: 727.1/ 727.2 |
| 162 | 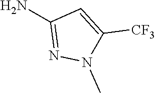 | 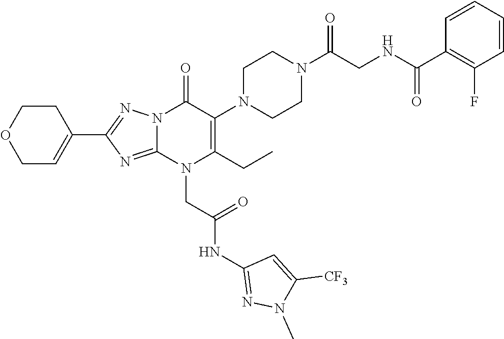 [SMILES: CCc1c(N2CCN(C(=O)CNC(=O)c3ccccc3F)CC2)c(=O)n2nc(C3=CCOCC3)nc2n1CC(=O)Nc1cc(C(F)(F)F)n(C)n1] | N-(2-(4-(2-(3,6-Dihydro-2H-pyran- 4-yl)-5-ethyl-4-(2-((1-methyl-5- (trifluoromethyl)-1H-pyrazol-3- yl)amino)-2-oxoethyl)-7-oxo-4,7- dihydro-[1,2,4]triazolo[1,5- a]pyrimidin-6-yl)piperazin-1-yl)-2- oxoethyl)-2-fluorobenzamide | [M − H]⁻: 713.3/ 713.2 |
| 163 | 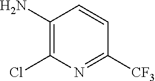 [SMILES: Nc1ccc(C(F)(F)F)nc1Cl] | 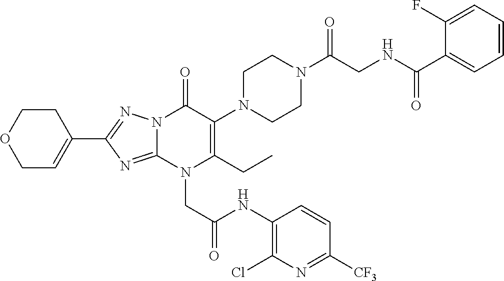 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)nc1Cl] | N-(2-(4-(4-(2-((2-Chloro-6- (trifluoromethyl)pyridin-3- yl)amino)-2-oxoethyl)-2-(3,6- dihydro-2H-pyran-4-yl)-5-ethyl-7- oxo-4,7-dihydro- [1,2,4]triazolo[1,5-a]pyrimidin-6- yl)piperazin-1-yl)-2-oxoethyl)-2- fluorobenzamide | [M − H]⁻: 744.2/ 744.1 |

### Example 164: 4-Chloro-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzamide

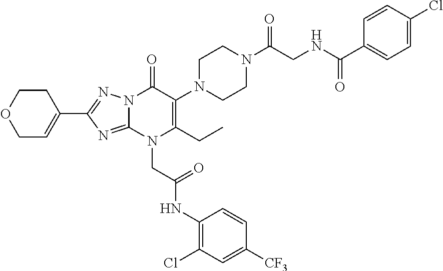 [SMILES: CCc1c(N2CCN(C(=O)CNC(=O)c3ccc(Cl)cc3)CC2)c(=O)n2nc(C3=CCOCC3)nc2n1CC(=O)Nc1ccc(C(F)(F)F)cc1Cl]

A mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 4-chlorobenzoyl chloride (9.3 mg, 0.05 mmol) and TEA (14.6 mg, 0.15 mmol) in DCM (3 mL) was stirred at r.t. for 1 h. The reaction mixture was concentrated and the residue was purified by Prep-HPLC on a C18 column eluting with MeCN/H₂O (30%-50%, with 0.1% FA) to afford the title compound (10.74 mg, 29.2% yield) as a white solid. ¹H NMR (400 MHZ, DMSO-d₆) δ ppm 10.36 (s, 1H), 8.71 (t, J=5.6 Hz, 1H), 8.06 (d, J=8.5 Hz, 1H), 7.97 (d, J=1.4 Hz, 1H), 7.91 (d, J=8.6 Hz, 2H), 7.72 (dd, J=8.7, 1.6 Hz, 1H), 7.57 (d, J=8.5 Hz, 2H), 6.83 (s, 1H), 5.32 (s, 2H), 4.39 (d, J=12.0 Hz, 1H), 4.25 (d, J=2.3 Hz, 2H), 4.20 (d, J=5.5 Hz, 2H), 3.96 (d, J=12.3 Hz, 1H), 3.80 (t, J=5.4 Hz, 2H), 3.57-3.35 (m, 2H), 3.24 (d, J=11.5 Hz, 1H), 3.00 (d, J=7.5 Hz, 2H), 2.85-2.79 (m, 1H), 2.73 (d, J=10.9 Hz, 2H), 2.51 (s, 2H), 1.20 (t, J=7.5 Hz, 3H). LCMS calc. for C₃₄H₃₄C₁₂F₃N₈O₅ [M+H]⁺: m/z=761.2; Found: 761.2.

### Example 165: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)cyclopropanecarboxamide

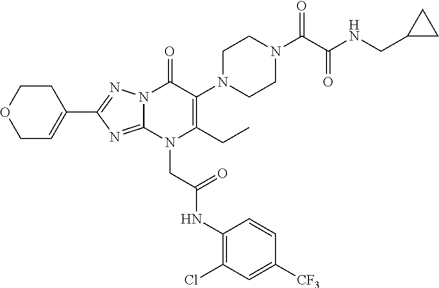 [SMILES: CCc1c(N2CCN(C(=O)C(=O)NCC3CC3)CC2)c(=O)n2nc(C3=CCOCC3)nc2n1CC(=O)Nc1ccc(C(F)(F)F)cc1Cl]

Method E: A mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (10 mg, 0.02 mmol, Example 38), cyclopropanecarbonyl chloride (2.18 mg, 0.02 mmol) and TEA (4.88 mg, 0.05 mmol) in CH₂Cl₂ (3 mL) was stirred at r.t. for 1 h. The reaction mixture was concentrated and the residue was purified by Prep-HPLC on a C18 column eluting with MeCN/H₂O (30%-50%, with 0.1% FA) to afford the title compound (3.55 mg, 32% yield) as a white solid. ¹H NMR (400 MHZ, DMSO-d₆)₈ ppm 10.32 (s, 1H), 8.22 (t, J=5.5 Hz, 1H), 8.06 (d, J=8.6 Hz, 1H), 7.97 (d, J=1.4 Hz, 1H), 7.72 (d, J=8.7 Hz, 1H), 6.83 (s, 1H), 5.32 (s, 2H), 4.37 (d, J=11.4 Hz, 1H), 4.25 (d, J=2.2 Hz, 2H), 4.10-3.93 (m, 2H), 3.82 (dd, J=17.0, 11.7 Hz, 3H), 3.53-3.34 (m, 3H), 3.20 (t, J=11.4 Hz, 1H), 2.98 (d, J=7.4 Hz, 2H), 2.74 (dd, J=33.1, 10.7 Hz, 4H), 1.77-1.68 (m, 1H), 1.26-1.14 (m, 3H), 0.73-0.59 (m, 4H). LCMS calc. for C₃₁H₃₅ClF₃N₈O₅ [M+H]⁺: m/z=691.2; Found: 691.2.

The following compounds listed in Table 6 were prepared by using an appropriate chloride and Example 38 as the methods analogous to those described for Method E.

TABLE 6
Preparations of Examples (Ex)

| | | | | LCMS |
|---|---|---|---|---|
| | | | | Cacl./ |
| Ex | Chloride | Structure | Name | Found |
| 166 | 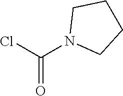 | 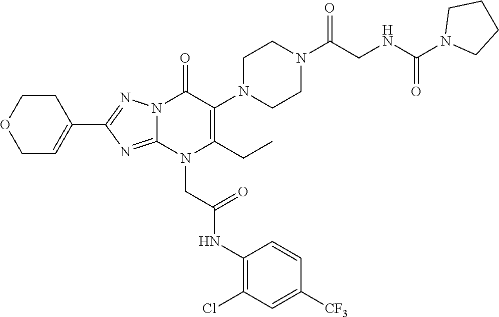 [SMILES: CCc1c(N2CCN(C(=O)CNC(=O)N3CCCC3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)pyrrolidine-1- carboxamide | [M − H]⁺: 720.3/ 720.2 |
| 167 | 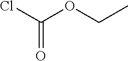 | 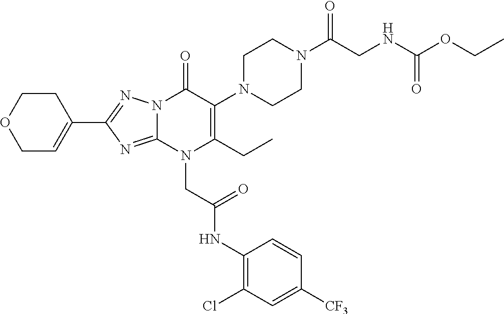 [SMILES: CCOC(=O)NCC(=O)N1CCN(c2c(CC)n(CC(=O)Nc3ccc(C(F)(F)F)cc3Cl)c3nc(C4=CCOCC4)nn3c2=O)CC1] | Ethyl (2-(4-(4-(2-((2-chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)carbamate | [M + H]⁺: 695.2/ 695.2 |
| 168 | 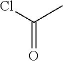 | 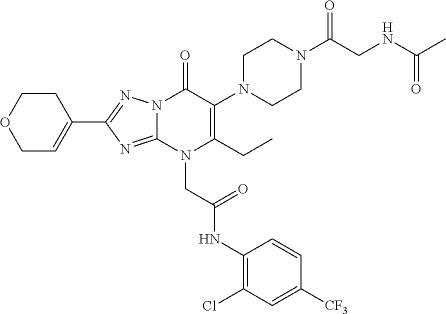 [SMILES: CCc1c(N2CCN(C(=O)CNC(C)=O)CC2)c(=O)n2nc(C3=CCOCC3)nc2n1CC(=O)Nc1ccc(C(F)(F)F)cc1Cl] | 2-(6-(4-(Acetylglycyl)piperazin-1- yl)-2-(3,6-dihydro-2H-pyran-4-yl)- 5-ethyl-7-oxo-[1,2,4]triazolo[1,5- a]pyrimidin-4(7H)-yl)-N-(2-chloro- 4- (trifluoromethyl)phenyl)acetamide | [M + H]⁺: 665.2/ 664.6 |
| 169 | 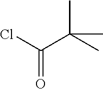 | 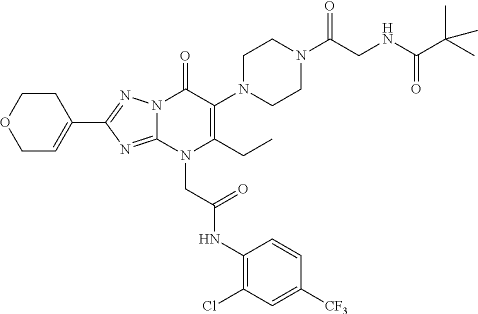 [SMILES: CCc1c(N2CCN(C(=O)CNC(=O)C(C)(C)C)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)pivalamide | [M + H]⁺: 707.3/ 706.8 |
| 170 | 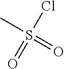 | 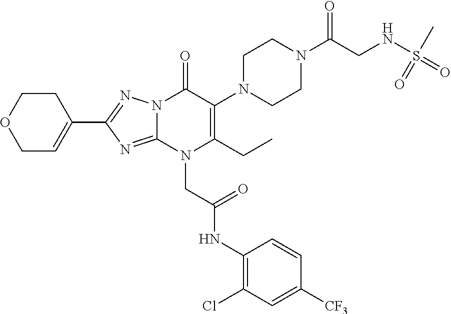 [SMILES: CCc1c(N2CCN(C(=O)CNS(C)(=O)=O)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4- ((methylsulfonyl)glycyl)piperazin- 1-yl)-7-oxo-[1,2,4]triazolo[1,5- a]pyrimidin-4(7H)-yl)acetamide | [M + H]⁺: 701.2/ 701.2 |
| 171 | 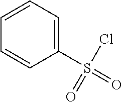 | 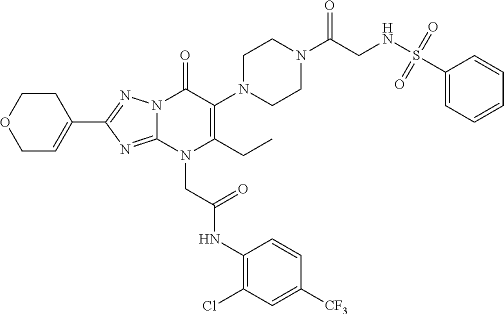 [SMILES: CCc1c(N2CCN(C(=O)CNS(=O)(=O)c3ccccc3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-7- oxo-6-(4-(2- (phenylsulfonamido)acetyl)piperazin- 1-yl)-[1,2,4]triazolo[1,5- a]pyrimidin-4(7H)-yl)acetamide | [M + H]⁺: 763.2/ 763.1 |
| 172 | 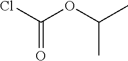 | 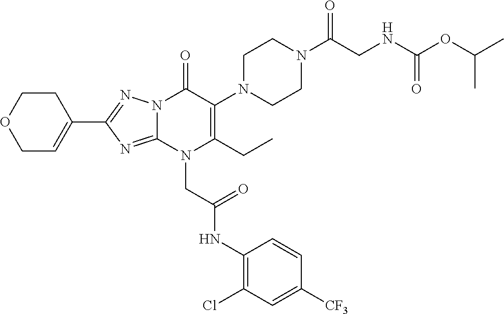 [SMILES: CCc1c(N2CCN(C(=O)CNC(=O)OC(C)C)CC2)c(=O)n2nc(C3=CCOCC3)nc2n1CC(=O)Nc1ccc(C(F)(F)F)cc1Cl] | Isopropyl (2-(4-(4-(2-((2-chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5-a] pyrimidin-6-yl)piperazin-1-yl)-2- oxoethyl)carbamate | [M + H]⁺: 709.2/ 709.0 |
| 173 | 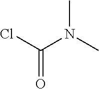 | 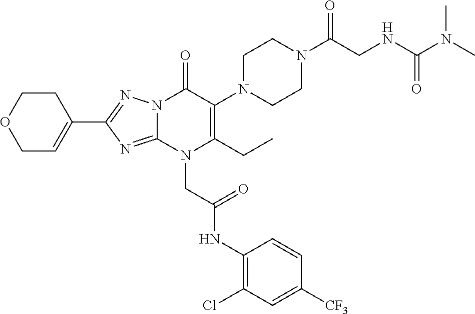 [SMILES: CCc1c(N2CCN(C(=O)CNC(=O)N(C)C)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-6-(4- ((dimethylcarbamoyl)glycyl) piperazin-1-yl)-5-ethyl-7-oxo- [1,2,4]triazolo[1,5-a]pyrimidin- 4(7H)-yl)acetamide | [M + H]⁺: 694.2/ 694.5 |
| 174 | 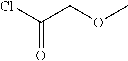 | 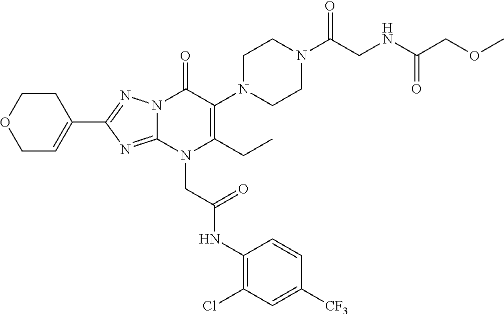 [SMILES: CCc1c(N2CCN(C(=O)CNC(=O)COC)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4-((2- methoxyacetyl)glycyl)piperazin-1- yl)-7-oxo-[1,2,4]triazolo[1,5- a]pyrimidin-4(7H)-yl)acetamide | [M + H]⁺: 695.2/ 695.1 |
| 175 | 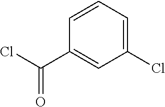 | 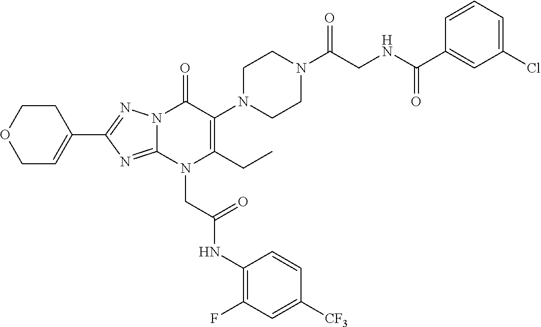 | 3-Chloro-N-(2-(4-(4-(2-((2-chloro- 4-(trifluoromethyl)phenyl)amino)- 2-oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)benzamide | [M + H]⁺: 761.2/ 761.3 |
| 176 | 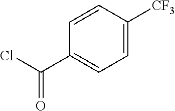 [SMILES: O=C(Cl)c1ccc(C(F)(F)F)cc1] | 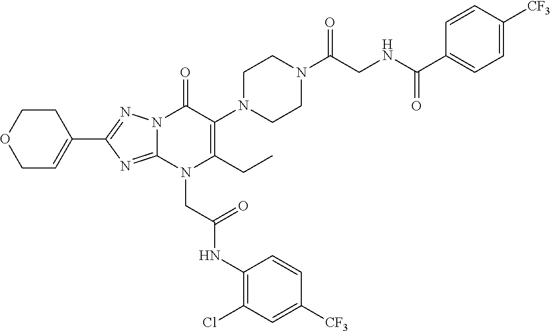 [SMILES: CCc1c(N2CCN(C(=O)CNC(=O)c3ccc(C(F)(F)F)cc3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]Pyrimidin-6-yl)piperazin-1-yl)-2- oxoethyl)-4- (trifluoromethyl)benzamide | [M + H]⁺: 795.2/ 795.2 |
| 177 | 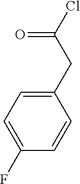 [SMILES: O=C(Cl)Cc1ccc(F)cc1] | 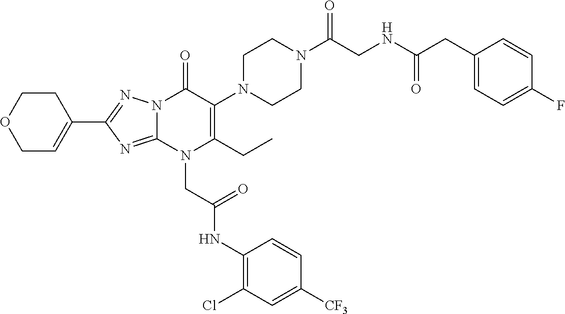 [SMILES: CCc1c(N2CCN(C(=O)CNC(=O)Cc3ccc(F)cc3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4-((2-(4- fluorophenyl)acetyl)glycyl)piperazin- 1-yl)-7-oxo-[1,2,4]triazolo[1,5- a]pyrimidin-4(7H)-yl)acetamide | [M + H]⁺: 759.2/ 759.2 |

TABLE 7
¹H NMR of Examples (Ex)

| Ex | ¹H NMR (400 MHz, DMSO-d₆) δ ppm |
|---|---|
| 166 | 10.36 (s, 1H), 8.06 (d, J = 8.5 Hz, 1H), 7.97 (d, J = 2.1 Hz, 1H), 7.72 (dd, J = 8.8, 2.2 Hz, 1H), 6.83 (dd, |
| | J = 3.2, 1.7 Hz, 1H), 6.08 (t, J = 5.6 Hz, 1H), 5.32 (s, 2H), 4.37 (d, J = 12.4 Hz, 1H), 4.25 (q, J = 2.9 Hz, |
| | 2H), 3.91 (d, J = 5.2 Hz, 2H), 3.80 (t, J = 5.5 Hz, 2H), 3.46 (s, 3H), 3.24 (dd, J = 10.5, 3.2 Hz, 5H), 2.98 |
| | (d, J = 7.6 Hz, 2H), 2.74 (dd, J = 10.6 Hz, 5H), 1.81 (p, J = 3.6 Hz, 4H), 1.19 (t, J = 7.4 Hz, 3H). |
| 167 | 10.36 (s, 1H), 8.06 (d, J = 8.6 Hz, 1H), 7.97 (d, J = 2.0 Hz, 1H), 7.72 (dd, J = 8.6, 2.1 Hz, 1H), 7.07 (t, |
| | J = 5.9 Hz, 1H), 6.83 (dd, J = 3.3, 1.8 Hz, 1H), 5.32 (s, 2H), 4.36 (d, J = 12.6 Hz, 1H), 4.25 (q, J = 2.9 Hz, |
| | 2H), 4.03-3.97 (m, 2H), 3.92 (d, J = 5.7 Hz, 1H), 3.87-3.76 (m, 4H), 3.46 (t, J = 11.6 Hz, 2H), 3.19 (t, |
| | J = 12.4 Hz, 2H), 2.98 (d, J = 7.6 Hz, 2H), 2.81-2.65 (m, 4H), 1.18 (td, J = 7.2, 5.3 Hz, 6H). |
| 168 | 10.35 (s, 1H), 8.06 (d, J = 8.5 Hz, 1H), 7.99 (t, J = 5.5 Hz, 1H), 7.96 (d, J = 1.4 Hz, 1H), 7.74-7.68 (m, |
| | 1H), 6.83 (s, 1H), 5.32 (s, 2H), 4.37 (d, J = 11.8 Hz, 1H), 4.25 (d, J = 2.5 Hz, 2H), 3.98 (dd, J = 12.2, 5.5 |
| | Hz,2H), 3.82 (dd, J = 16.7, 11.4 Hz, 3H), 3.44 (dd, J = 26.5, 16.2 Hz, 3H), 3.19 (d, J = 11.3 Hz, 1H), |
| | 3.02-2.94 (m, 2H), 2.74(dd, J = 32.1, 11.4 Hz, 4H), 1.88 (s, 3H), 1.19 (t, J = 7.4 Hz, 3H). |
| 169 | 10.32 (s, 1H), 8.06 (d, J = 8.5 Hz, 1H), 7.96 (s, 1H), 7.72 (d, J = 7.1 Hz, 1H), 7.52 (t, J = 5.4 Hz, 1H), |
| | 6.83 (s, 1H), 5.32 (s, 2H), 4.37 (d, J = 12.2 Hz, 1H), 4.25 (d, J = 2.5 Hz, 2H), 3.95 (t, J = 6.2 Hz, 2H), |
| | 3.87 (d, J = 12.2 Hz, 1H), 3.80 (t, J = 5.4 Hz, 2H), 3.45 (d, J = 11.7 Hz, 3H), 3.21 (d, J = 11.2 Hz, 1H), |
| | 2.99 (d, J = 7.2 Hz, 2H), 2.81-2.66 (m, 3H), 1.19 (t, J = 7.4 Hz, 3H), 1.13 (s, 10H). |
| 170 | 10.36 (s, 1H), 8.06 (d, J = 8.6 Hz, 1H), 7.97 (d, J = 1.4 Hz, 1H), 7.72 (d, J = 8.6 Hz, 1H), 7.10 (t, J = 5.6 |
| | Hz, 1H), 6.83 (s, 1H), 5.32 (s, 2H), 4.38 (d, J = 11.9 Hz, 1H), 4.25 (d, J = 2.5 Hz, 2H), 4.10-4.01 (m, |
| | 1H), 3.90 (dd, J = 16.7, 5.7 Hz, 1H), 3.80 (t, J = 5.4 Hz, 3H), 3.49 (t, J = 10.8 Hz, 1H), 3.38 (d, J = 12.0 |
| | Hz, 2H), 3.20 (t, J = 11.8 Hz, 1H), 2.98 (d, J = 12.2 Hz, 5H), 2.85-2.65 (m, 4H), 1.19 (t, J = 7.5 Hz, |
| | 3H). |
| 171 | 10.37 (s, 1H), 8.06 (d, J = 8.6 Hz, 1H), 7.97 (d, J = 2.1 Hz, 1H), 7.86-7.82 (m, 2H), 7.75 (d, J = 3.5 Hz, |
| | 1H), 7.72 (dd, J = 9.0, 2.1 Hz, 1H), 7.64-7.57 (m, 3H), 6.83 (p, J = 1.4 Hz, 1H), 5.31 (s, 2H), 4.25 (dt, |
| | J = 6.1, 3.3 Hz, 3H), 3.89 (d, J = 16.3 Hz, 1H), 3.82-3.71 (m, 4H), 3.44 (t, J = 11.8 Hz, 2H), 3.27 (s, 2H), |
| | 3.13 (t, J = 12.0 Hz, 1H), 2.96 (d, J = 7.7 Hz, 2H), 2.69 (s, 1H), 2.67-2.62 (m, 2H), 1.17 (t, J = 7.4 Hz, |
| | 3H). |
| 172 | 10.35 (s, 1H), 8.06 (d, J = 8.5 Hz, 1H), 7.97 (d, J = 1.6 Hz, 1H), 7.72 (dd, J = 8.7, 1.8 Hz, 1H), 6.99 (t, |
| | J = 5.8 Hz, 1H), 6.83 (s, 1H), 5.32 (s, 2H), 4.75 (dt, J = 12.5, 6.2 Hz, 1H), 4.36 (d, J = 11.4 Hz, 1H), 4.25 |
| | (d, J = 2.5 Hz, 2H), 3.91 (d, J = 5.7 Hz, 1H), 3.87-3.78 (m, 4H), 3.45 (d, J = 10.7 Hz, 2H), 3.24 (d, |
| | J = 9.0 Hz, 2H), 2.98 (d, J = 7.4 Hz, 2H), 2.84-2.64 (m, 4H), 1.18 (d, J = 6.3 Hz, 9H). |
| 173 | 10.36 (s, 1H), 8.06 (d, J = 8.4 Hz, 1H), 7.96 (d, J = 1.5 Hz, 1H), 7.72 (dd, J = 8.7, 1.7 Hz, 1H), 6.83 (s, |
| | 1H), 6.32 (t, J = 5.5 Hz, 1H), 5.32 (s, 2H), 4.37 (d, J = 12.3 Hz, 1H), 4.25 (d, J = 2.5 Hz, 2H), 3.90 (d, |
| | J = 5.1 Hz, 3H), 3.80 (t, J = 5.4 Hz, 2H), 3.44 (dd, J = 26.2, 15.3 Hz, 2H), 3.35 (s, 2H), 3.19 (t, J = 11.4 |
| | Hz, 1H), 3.01 (s, 2H), 2.82 (s, 6H), 2.78 (d, J = 11.7 Hz, 1H), 2.70 (d, J = 11.3 Hz, 2H), 1.19 (t, J = 7.5 |
| | Hz, 3H). |
| 175 | 10.36 (s, 1H), 8.78 (t, J = 5.7 Hz, 1H), 8.06 (d, J = 8.5 Hz, 1H), 7.94 (dd, J = 8.8, 6.9 Hz, 2H), 7.85 (dd, |
| | J = 7.8, 1.4 Hz, 1H), 7.72 (d, J = 7.1 Hz, 1H), 7.65-7.62 (m, 1H), 7.53 (d, J = 7.9 Hz, 1H), 6.84 (s, 1H), |
| | 5.32 (s, 2H), 4.38 (d, J = 12.5 Hz, 1H), 4.23 (dd, J = 20.7, 3.6 Hz, 4H), 3.96 (d, J = 11.5 Hz, 1H), 3.81 (t, |
| | J = 5.5 Hz, 2H), 3.45 (d, J = 53.6 Hz, 2H), 3.30-3.20 (m, 4H), 3.00 (d, J = 7.4 Hz, 2H), 2.77 (d, |
| | J = 23.3 Hz, 2H), 1.20 (t, J = 7.5 Hz, 3H). |
| 176 | 10.39 (s, 1H), 8.88 (s, 1H), 8.08 (t, J = 9.6 Hz, 3H), 7.97 (s, 1H), 7.89 (d, J = 7.9 Hz, 2H), 7.73 (d, |
| | J = 8.1 Hz, 1H), 6.84 (s, 1H), 5.33 (s, 2H), 4.39 (d, J = 11.1 Hz, 1H), 4.26 (s, 4H), 3.97 (d, J = 12.0 Hz, 1H), |
| | 3.81 (s, 2H), 3.56-3.48 (m, 2H), 3.06 (dd, J = 20.2, 13.0 Hz, 4H), 2.89-2.76 (m, 2H), 2.74 (d, J = 10.1 |
| | Hz, 2H), 1.19 (d, J = 7.2 Hz, 3H). |
| 177 | 10.35 (s, 1H), 8.19 (t, J = 5.5 Hz, 1H), 8.06 (d, J = 8.6 Hz, 1H), 7.97 (d, J = 1.5 Hz, 1H), 7.74-7.70 (m, |
| | 1H), 7.33 (dd, J = 8.6, 5.7 Hz, 2H), 7.15-7.09 (m, 2H), 6.83 (s, 1H), 5.32 (s, 2H), 4.37 (d, J = 11.9 Hz, |
| | 1H), 4.25 (d, J = 2.4 Hz, 2H), 4.07-3.95 (m, 2H), 3.81 (dd, J = 11.4, 6.2 Hz, 3H), 3.51 (s, 2H), 3.46 (t, |
| | J = 11.1 Hz, 1H), 3.37 (d, J = 13.9 Hz, 2H), 3.18 (t, J = 11.1 Hz, 1H), 2.98 (dd, J = 14.5, 6.9 Hz, 2H), |
| | 2.81-2.65 (m, 4H) , 1.18 (t, J = 7.5 Hz, 3H). |

### Example 178: N-(2-Chloro-4-(trifluoromethyl)phenyl)-2-(2-(3,6-dihydro-2H-pyran-4-yl)-5-ethyl-7-oxo-6-(4-((phenylcarbamoyl)glycyl)piperazin-1-yl)-[1,2,4]triazolo[1,5-a]pyrimidin-4 (7H)-yl)acetamide

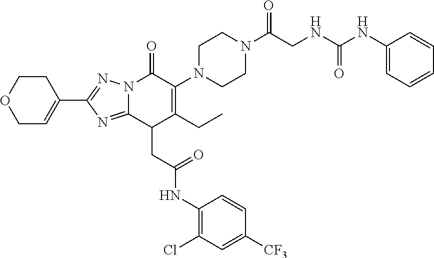 [SMILES: CCC1=C(N2CCN(C(=O)CNC(=O)Nc3ccccc3)CC2)C(=O)n2nc(C3=CCOCC3)nc2C1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (25 mg, 0.04 mmol, Example 38) and isocyanatobenzene (6 mg, 0.04 mmol) in MeCN (1.5 mL) was added DIEA (16 mg, 0.12 mmol). The mixture was stirred at r.t. overnight, and then concentrated under reduced pressure. The residue was purified by Prep-HPLC on a C18 column eluting with MeCN/H₂O (45-51%, with 0.1% FA) to afford the title compound (11.7 mg, 41% yield) as a white solid. ¹H NMR (400 MHZ, DMSO-d₆) δ ppm 10.36 (s, 1H), 8.90 (s, 1H), 8.10-8.04 (m, 1H), 7.97 (d, J=2.1 Hz, 1H), 7.72 (dd, J=8.8, 2.1 Hz, 1H), 7.44-7.36 (m, 2H), 7.26-7.19 (m, 2H), 6.89 (tt, J=7.4, 1.2 Hz, 1H), 6.83 (dq, J=3.0, 1.5 Hz, 1H), 6.36 (s, 1H), 5.34-5.18 (m, 2H), 4.40 (d, J=12.5 Hz, 1H), 4.26 (p, J=3.2 Hz, 2H), 4.13-3.96 (m, 2H), 3.88-3.74 (m, 3H), 3.47 (d, J=11.3 Hz, 1H), 3.40 (d, J=11.1 Hz, 2H), 3.24 (d, J=12.4 Hz, 2H), 2.99 (q, J=7.4 Hz, 2H), 2.80 (d, J=5.7 Hz, 1H), 2.72 (d, J=11.5 Hz, 2H), 1.19 (t, J=7.5 Hz, 3H). LCMS calc. for C₃₄H₃₆ClF₃N₉O₅ [M+H]⁺: m/z=742.2; Found: 742.0.

### Example 179: N-(2-Chloro-4-(trifluoromethyl)phenyl)-2-(6-(4-(((3,5-difluorophenyl) carbamoyl)glycyl)piperazin-1-yl)-2-(3,6-dihydro-2H-pyran-4-yl)-5-ethyl-7-oxo-[1,2,4]triazolo[1,5-a]pyrimidin-4(7H)-yl)acetamide

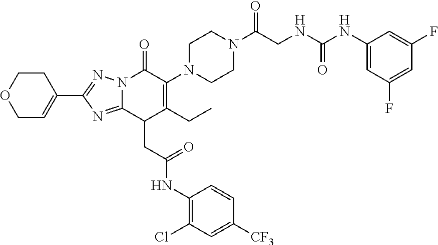 [SMILES: CCC1=C(N2CCN(C(=O)CNC(=O)Nc3cc(F)cc(F)c3)CC2)C(=O)n2nc(C3=CCOCC3)nc2C1CC(=O)Nc1ccc(C(F)(F)F)cc1Cl]

A mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (25 mg, 0.04 mmol, Example 38), 1,3-difluoro-5-isocyanatobenzene (10 mg, 0.06 mmol) and pyridine (13 mg, 0.16 mmol) in DCM (3 mL) was stirred at r.t. overnight, and then concentrated under reduced pressure. The residue was purified by Prep-HPLC on a C18 column eluting with MeCN/H₂O (55-70%, with 0.1% FA) to afford the title compound (11 mg, 40% yield) as a white solid. ¹H NMR (400 MHZ, DMSO-d₆) δ ppm 10.37 (s, 1H), 9.43 (s, 1H), 8.06 (d, J=8.6 Hz, 1H), 7.96 (d, J=2.1 Hz, 1H), 7.72 (dd, J=8.8, 2.2 Hz, 1H), 7.17-7.08 (m, 2H), 6.87-6.81 (m, 1H), 6.75-6.67 (m, 1H), 6.57 (t, J=4.9 Hz, 1H), 5.32 (s, 2H), 4.40 (d, J=12.4 Hz, 1H), 4.25 (d, J=3.1 Hz, 2H), 4.13-3.96 (m, 2H), 3.80 (t, J=5.4 Hz, 3H), 3.55-3.37 (m, 4H), 3.27-3.16 (m, 2H), 2.99 (d, J=7.6 Hz, 2H), 2.72 (d, J=11.1 Hz, 2H), 1.19 (t, J=7.5 Hz, 3H). LCMS calc. for C₃₄H₃₄ClF₅N₉O₅ [M+H]⁺: m/z=778.2; Found: 778.2.

### Example 180: N-(2-Chloro-4-(trifluoromethyl)phenyl)-2-(2-(3,6-dihydro-2H-pyran-4-yl)-5-ethyl-6-(4-(((2-fluorophenyl) carbamoyl)glycyl)piperazin-1-yl)-7-oxo-[1,2,4]triazolo[1,5-a]pyrimidin-4(7H)-yl)acetamide

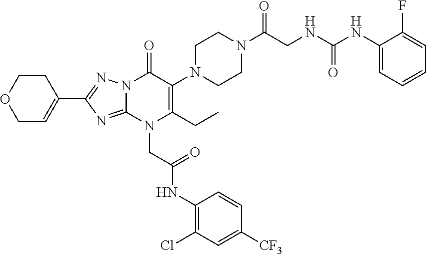 [SMILES: CCc1c(N2CCN(C(=O)CNC(=O)Nc3ccccc3F)CC2)c(=O)n2nc(C3=CCOCC3)nc2n1CC(=O)Nc1ccc(C(F)(F)F)cc1Cl]

This compound was prepared by the procedure analogous to that described for Example 179 using 1-fluoro-2-isocyanatobenzene to replace 1,3-difluoro-5-isocyanatobenzene to afford the title product as a white solid. ¹H NMR (400 MHZ, DMSO-d₆) δ ppm 10.37 (s, 1H), 8.74 (d, J=2.5 Hz, 1H), 8.12 (td, J=8.3, 1.8 Hz, 1H), 8.06 (d, J=8.5 Hz, 1H), 7.97 (d, J=2.1 Hz, 1H), 7.72 (dd, J=8.8, 2.1 Hz, 1H), 7.18 (ddd, J=11.7, 8.2, 1.5 Hz, 1H), 7.08 (td, J=7.8, 1.5 Hz, 1H), 6.97-6.90 (m, 2H), 6.83 (dp, J=2.9, 1.5 Hz, 1H), 5.32 (s, 2H), 4.40 (d, J=12.4 Hz, 1H), 4.25 (d, J=2.8 Hz, 2H), 4.16-3.98 (m, 2H), 3.87-3.78 (m, 3H), 3.51 (d, J=11.6 Hz, 3H), 3.23 (t, J=12.4 Hz, 2H), 3.06-2.93 (m, 2H), 2.81 (dd, J=14.1, 10.3 Hz, 1H), 2.72 (d, J=11.1 Hz, 2H), 1.20 (t, J=7.4 Hz, 3H). LCMS calc. for C₃₄H₃₅ClF₄N₉O₅ [M+H]⁺: m/z=760.2; Found: 760.2.

### Example 181: N-(2-Chloro-4-(trifluoromethyl)phenyl)-2-(6-(4-(((2-chlorophenyl) carbamoyl)glycyl)piperazin-1-yl)-2-(3,6-dihydro-2H-pyran-4-yl)-5-ethyl-7-oxo-[1,2,4]triazolo[1,5-a]pyrimidin-4(7H)-yl)acetamide

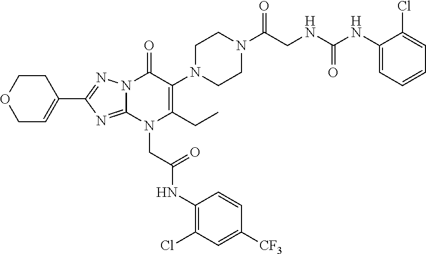 [SMILES: CCc1c(N2CCN(C(=O)CNC(=O)Nc3ccccc3Cl)CC2)c(=O)n2nc(C3=CCOCC3)nc2n1CC(=O)Nc1ccc(C(F)(F)F)cc1Cl]

This compound was prepared by the procedure analogous to that described for Example 179 using 1-chloro-2-isocyanatobenzene to replace 1,3-difluoro-5-isocyanatobenzene to afford the title product as a white solid. ¹H NMR (400 MHZ, DMSO-d₆) δ ppm 10.37 (s, 1H), 8.45 (s, 1H), 8.12 (dd, J=8.3, 1.6 Hz, 1H), 8.06 (d, J=8.6 Hz, 1H), 7.96 (d. J=2.2 Hz, 1H), 7.72 (dd, J=8.8, 2.2 Hz, 1H), 7.40 (dd, J=8.0, 1.6 Hz, 1H), 7.34 (t. J=5.0 Hz, 1H), 7.28-7.20 (m, 1H), 6.96 (td, J=7.6, 1.6 Hz, 1H), 6.89-6.80 (m, 1H), 5.32 (s, 2H), 4.41 (d. J=12.5 Hz, 1H), 4.25 (d. J=3.0 Hz, 2H), 4.19-4.00 (m, 2H), 3.87-3.76 (m, 3H), 3.54-3.35 (m, 3H), 3.27 (d, J=28.4 Hz, 3H), 2.99 (d. J=7.6 Hz, 2H), 2.73 (d. J=11.2 Hz, 2H), 1.20 (t. J=7.5 Hz, 3H). LCMS calc. for C₃H₃₅C₁₂F₃N₉O₅ [M+H]⁺: m/z=776.2; Found: 776.2. Example 182: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) indoline-1-carboxamide

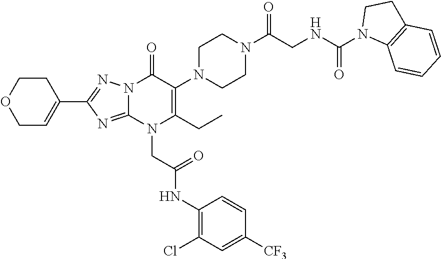 [SMILES: CCc1c(N2CCN(C(=O)CNC(=O)N3CCc4ccccc43)CC2)c(=O)n2nc(C3=CCOCC3)nc2n1CC(=O)Nc1ccc(C(F)(F)F)cc1Cl]

Method F: To a solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38) in DCM (1 mL) was added sat. NaHCO₃ aq. (0.5 mL) and triphosgene (5.80 mg, 0.02 mmol) at 0° C. The mixture was stirred at 0° C. for 0.5 h. and then indoline (6.89 mg, 0.06 mmol) was added. The mixture was stirred at r.t. for 2 h., diluted with H₂O (5 mL) and extracted with DCM (5 mL×3). The combined organic layers were washed with aq. NH₄Cl (10 mL), brine (10 mL), dried over anhydrous Na₂SO₄, filtered and concentrated under reduced pressure. The residue was purified by Prep-HPLC on a C18 column eluting with MeCN/H₂O (5-95%, with 0.1% NH₄HCO₃) to afford the title compound (7.3 mg, 19% yield) as a white solid. ¹H NMR (400 MHZ, DMSO-d₆)₈ ppm 10.36 (s, 1H), 8.06 (d, J=8.4 Hz, 1H), 7.97 (s, 1H), 7.82 (d, J=8.0 Hz, 1H), 7.72 (d, J=7.1 Hz, 1H), 7.16 (d, J=7.5 Hz, 1H), 7.08 (t, J=7.8 Hz, 1H), 6.84 (dd, J=8.3, 5.4 Hz, 2H), 6.72 (t, J=5.5 Hz, 1H), 5.32 (s, 2H), 4.39 (d, J=11.9 Hz, 1H), 4.26 (d, J=2.5 Hz, 2H), 4.02 (d, J=5.5 Hz, 2H), 3.94 (t, J=8.5 Hz, 3H), 3.81 (t, J=5.4 Hz, 2H), 3.49 (d, J=10.4 Hz, 2H), 3.24 (m, 3H), 3.15 (t, J=8.7 Hz, 2H), 3.00 (d, J=7.3 Hz, 2H), 2.81 (d, J=7.8 Hz, 1H), 2.72 (d, J=11.5 Hz, 2H), 1.20 (t, J=7.5 Hz, 3H). LCMS calc. for C₃₆H₃₈ClF₃N₉O₅ [M+H]⁺: m/z=768.3; Found: 768.2.

The following compounds listed in Table 8 were prepared by using an appropriate chloride and Example 38 as the methods analogous to those described for Method F.

TABLE 8
Preparations of Examples (Ex)

| | | | | LCMS |
|---|---|---|---|---|
| | | | | Cacl./ |
| Ex | Chloride | Structure | Name | Found |
| 183 | 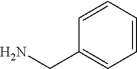 | 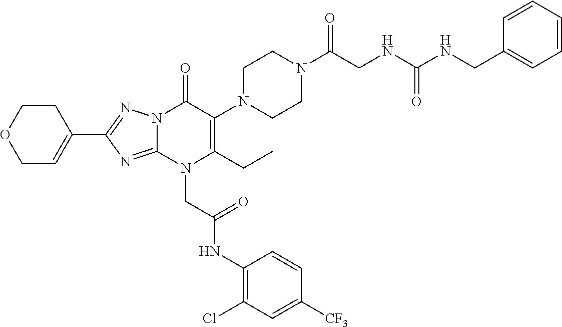 [SMILES: CCc1c(N2CCN(C(=O)CNC(=O)NCc3ccccc3)CC2)c(=O)n2nc(C3=CCOCC3)nc2n1CC(=O)Nc1ccc(C(F)(F)F)cc1Cl] | 2-(6-(4- ((Benzylcarbamoyl)glycyl)piperazin- 1-yl)-2-(3,6-dihydro-2H-pyran-4- yl)-5-ethyl-7-oxo- [1,2,4]triazolo[1,5-a]pyrimidin- 4(7H)-yl)-N-(2-chloro-4- (trifluoromethyl)phenyl)acetamide | [M + H]⁺: 756.3/ 756.2 |
| 184 | 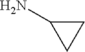 | 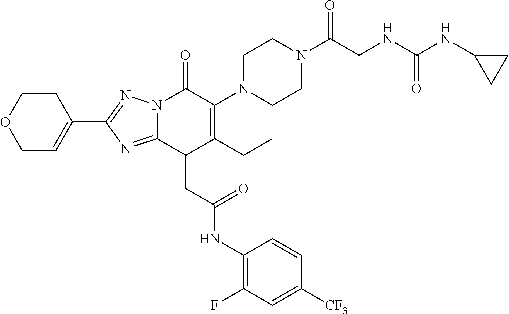 [SMILES: CCC1=C(N2CCN(C(=O)CNC(=O)NC3CC3)CC2)C(=O)n2nc(C3=CCOCC3)nc2C1CC(=O)Nc1ccc(C(F)(F)F)cc1F] | N-(2-Chloro-4- (trifluoromethyl)phenyl)-2-(6-(4- ((cyclopropylcarbamoyl)glycyl) piperazin-1-yl)-2-(3,6-dihydro-2H- pyran-4-yl)-5-ethyl-7-oxo- [1,2,4]triazolo[1,5-a]pyrimidin- 4(7H)-yl)acetamide | [M + H]⁺: 706.2/ 706.2 |
| 185 | 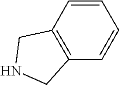 | 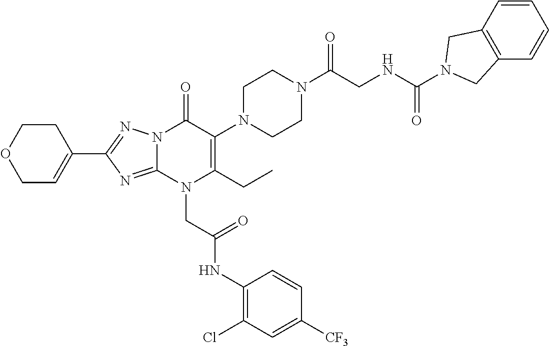 [SMILES: CCc1c(N2CCN(C(=O)CNC(=O)N3Cc4ccccc4C3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)isoindoline-2- carboxamide | [M + H]⁺: 768.3/ 768.5 |
| 186 | 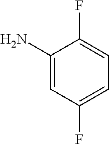 | 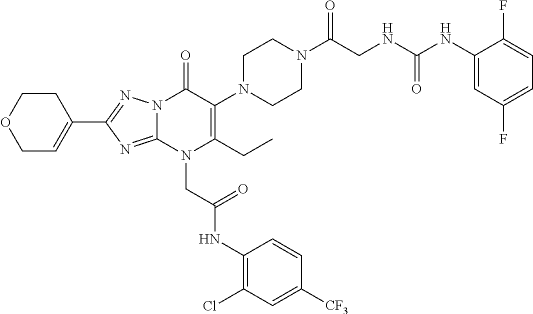 [SMILES: CCc1c(N2CCN(C(=O)CNC(=O)Nc3cc(F)ccc3F)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(6-(4- (((2,5- difluorophenyl)carbamoyl)glycyl) piperazin-1-yl)-2-(3,6-dihydro-2H- pyran-4-yl)-5-ethyl-7-oxo- [1,2,4]triazolo[1,5-a]pyrimidin- 4(7H)-yl)acetamide | [M + H]⁺: 778.2/ 778.6 |
| 187 | 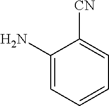 | 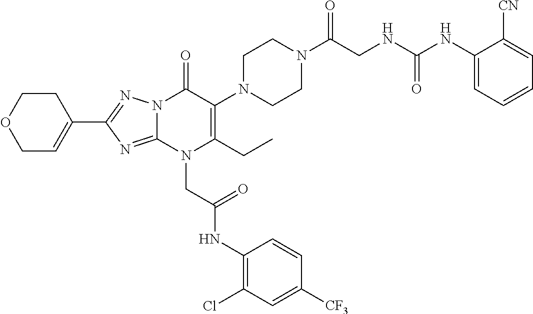 [SMILES: CCc1c(N2CCN(C(=O)CNC(=O)Nc3ccccc3C#N)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(6-(4- (((2- cyanophenyl)carbamoyl)glycyl) piperazin-1-yl)-2-(3,6-dihydro-2H- pyran-4-yl)-5-ethyl-7-oxo- [1,2,4]triazolo[1,5-a]pyrimidin- 4(7H)-yl)acetamide | [M + H]⁺: 767.2/ 767.5 |
| 188 | 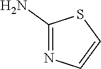 | 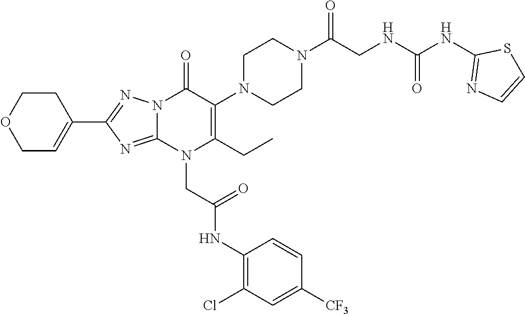 [SMILES: CCc1c(N2CCN(C(=O)CNC(=O)Nc3nccs3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-7- oxo-6-(4-((thiazol-2- ylcarbamoyl)glycyl)piperazin-1-yl)- [1,2,4]triazolo[1,5-a]pyrimidin- 4(7H)-yl)acetamide | [M + H]⁺: 749.2/ 749.2 |
| 189 | 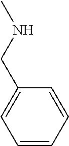 | 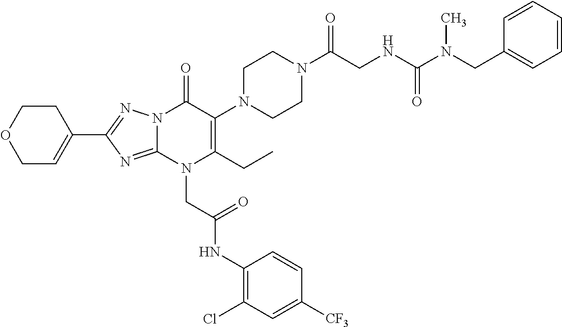 [SMILES: CCc1c(N2CCN(C(=O)CNC(=O)N(C)Cc3ccccc3)CC2)c(=O)n2nc(C3=CCOCC3)nc2n1CC(=O)Nc1ccc(C(F)(F)F)cc1Cl] | 2-(6-(4- ((Benzyl(methyl)carbamoyl)glycyl) piperazin-1-yl)-2-(3,6-dihydro-2H- pyran-4-yl)-5-ethyl-7-oxo- [1,2,4]triazolo[1,5-a]pyrimidin- 4(7H)-yl)-N-(2-chloro-4- (trifluoromethyl)phenyl)acetamide | [M + H]⁺: 770.3/ 770.7 |
| 190 | 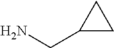 | 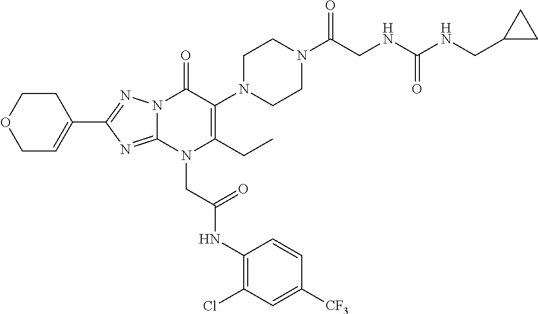 [SMILES: CCc1c(N2CCN(C(=O)CNC(=O)NCC3CC3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(6-(4- (((cyclopropylmethyl)carbamoyl) glycyl)piperazin-1-yl)-2-(3,6- dihydro-2H-pyran-4-yl)-5-ethyl-7- oxo-[1,2,4]triazolo[1,5- a]pyrimidin-4(7H)-yl)acetamide | [M + H]⁺: 720.3/ 720.2 |
| 191 | 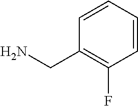 | 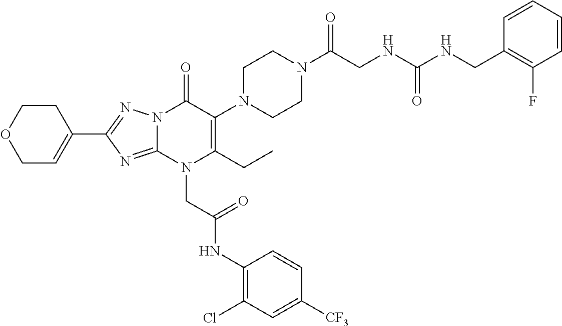 [SMILES: CCc1c(N2CCN(C(=O)CNC(=O)NCc3ccccc3F)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4-(((2- fluorobenzyl)carbamoyl)glycyl) piperazin-1-yl)-7-oxo- [1,2,4]triazolo[1,5-a]pyrimidin- 4(7H)-yl)acetamide | [M + H]⁺: 774.2/ 774.2 |
| 192 | 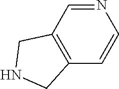 | 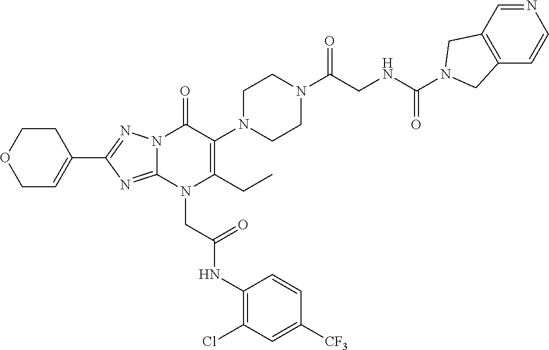 [SMILES: CCc1c(N2CCN(C(=O)CNC(=O)N3Cc4ccncc4C3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-1,3-dihydro-2H- pyrrolo[3,4-c]pyridine-2- carboxamide | [M + H]⁺: 769.3/ 769.2 |
| 193 | 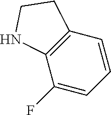 | 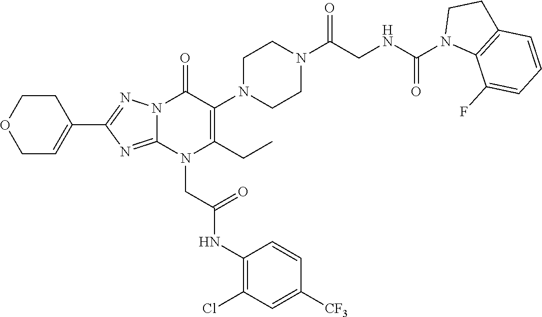 [SMILES: CCc1c(N2CCN(C(=O)CNC(=O)N3CCc4cccc(F)c43)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-7-fluoroindoline-1- carboxamide | [M + H]⁺: 786.2/ 786.2 |

TABLE 9
¹H NMR of Examples (Ex)

| Ex | ¹H NMR (400 MHz, DMSO-d₆) δ ppm |
|---|---|
| 183 | 10.35 (s, 1H), 8.06 (d, J = 8.4 Hz, 1H), 7.97 (s, 1H), 7.72 (d, J = 7.6 Hz, 1H), 7.34-7.29 (m, 2H), 7.28- |
| | 7.18 (m, 3H), 6.83 (s, 1H), 6.77 (t, J = 6.1 Hz, 1H), 6.13 (s, 1H), 5.32 (s, 2H), 4.39 (d, J = 11.7 Hz, 1H), |
| | 4.29-4.21 (m, 4H), 3.98 (d, J = 26.6 Hz, 2H), 3.81 (d, J = 5.4 Hz, 3H), 3.45 (d, J = 10.5 Hz, 2H), 3.19 |
| | (s, 2H), 2.99 (d, J = 7.8 Hz, 2H), 2.80 (d, J = 10.4 Hz, 2H), 2.71 (d, J = 10.1 Hz, 2H), 1.19 (t, J = 7.4 Hz, |
| | 3H). |
| 184 | 10.35 (s, 1H), 8.06 (d, J = 8.5 Hz, 1H), 7.96 (s, 1H), 7.72 (d, J = 7.2 Hz, 1H), 6.83 (s, 1H), 6.49 (d, |
| | J = 2.5 Hz, 1H), 5.97 (s, 1H), 5.32 (s, 2H), 4.38 (d, J = 12.0 Hz, 1H), 4.25 (d, J = 2.4 Hz, 2H), 4.02-3.86 (m, |
| | 2H), 3.80 (t, J = 5.4 Hz, 3H), 3.46 (t, J = 11.0 Hz, 1H), 3.38 (d, J = 9.7 Hz, 1H), 3.25-3.14 (m, 2H), 2.98 |
| | (d, J = 7.4 Hz, 2H), 2.82-2.65 (m, 4H), 2.44-2.39 (m, 1H), 1.19 (t, J = 7.5 Hz, 3H), 0.61-0.54 (m, 2H), |
| | 0.37-0.31 (m, 2H). |
| 185 | 10.37 (s, 1H), 8.06 (d, J = 8.4 Hz, 1H), 7.97 (d, J = 2.0 Hz, 1H), 7.72 (dd, J = 8.9, 2.2 Hz, 1H), 7.39- |
| | 7.32 (m, 2H), 7.32-7.25 (m, 2H), 6.83 (dq, J = 3.2, 1.5 Hz, 1H), 6.42 (t, J = 5.6 Hz, 1H), 5.32 (s, 2H), |
| | 4.64 (s, 4H), 4.39 (d, J = 12.4 Hz, 1H), 4.26 (d, J = 3.3 Hz, 2H), 4.05-3.96 (m, 2H), 3.92 (d, J = 13.0 |
| | Hz, 1H), 3.80 (t, J = 5.5 Hz, 2H), 3.49 (t, J = 11.7 Hz, 2H), 3.40-3.34 (m, 2H), 3.22 (s, 1H), 2.99 (d, |
| | J = 7.8 Hz, 2H), 2.80 (d, J = 12.2 Hz, 1H), 2.72 (d, J = 11.3 Hz, 2H), 1.20 (t, J = 7.5 Hz, 3H). |
| 186 | 10.36 (s, 1H), 8.99 (s, 1H), 8.06 (d, J = 8.8 Hz, 1H), 8.02 (d, J = 1.6 Hz, 1H), 7.97 (d, J = 1.5 Hz, 1H), |
| | 7.76-7.70 (m, 1H), 7.23 (ddd, J = 11.0, 9.1, 5.3 Hz, 1H), 7.08 (t, J = 4.8 Hz, 1H), 6.84 (s, 1H), 6.77-6.71 |
| | (m, 1H), 5.32 (s, 2H), 4.40 (d, J = 12.4 Hz, 2H), 4.26(d, J = 2.3 Hz, 2H), 4.12 (d, J = 4.3 Hz, 2H), 4.05 |
| | (t, J = 5.4 Hz, 2H), 3.82-3.79 (m, 4H), 2.99 (d, J = 7.4 Hz, 2H), 2.76 (dd, J = 25.7,11.8 Hz, 4H), 1.20(t, |
| | J = 7.5 Hz, 3H). |
| 187 | 10.39 (s, 1H), 9.02 (s, 1H), 8.06 (dd, J = 8.5, 4.7 Hz, 2H), 7.96 (s, 1H), 7.71 (ddd, J = 9.5, 8.2, 1.8 Hz, |
| | 2H), 7.59 (ddd, J = 8.8, 7.4, 1.6 Hz, 1H), 7.31 (s, 1H), 7.11 (td, J = 7.6, 1.1 Hz, 1H), 6.87-6.80 (m, 1H), |
| | 5.32 (s, 2H), 4.41 (d, J = 12.6 Hz, 1H), 4.25 (q, J = 2.9 Hz, 2H), 4.19-4.11 (m, 1H), 4.04 (dd, J = 17.2, |
| | 4.8 Hz, 1H), 3.80 (t, J = 5.5 Hz, 3H), 3.50 (s, 2H), 3.23 (s, 3H), 2.99 (d, J = 8.0 Hz, 2H), 2.83 (d, |
| | J = 12.0 Hz, 1H), 2.73 (d, J = 11.3 Hz, 2H), 1.20 (t, J = 7.5 Hz, 3H). |
| 188 | 10.69 (s, 1H), 10.37 (s, 1H), 8.06 (d, J = 8.5 Hz, 1H), 7.97 (d, J = 1.5 Hz, 1H), 7.72 (dd, J = 8.8, 1.5 Hz, |
| | 1H), 7.31 (d, J = 3.6 Hz, 1H), 7.02 (d, J = 3.6 Hz, 1H), 6.85 (d, J = 11.4 Hz, 2H), 5.32 (s, 2H), 4.39 (d, |
| | J = 12.0 Hz, 1H), 4.25 (d, J = 2.4 Hz, 2H), 4.10 (m, 2H), 3.80 (t, J = 5.4 Hz, 3H), 3.57-3.34 (m, 4H), |
| | 3.21 (d, J = 11.7 Hz, 2H), 3.03-2.94 (m, 2H), 2.73 (d, J = 11.0 Hz, 2H), 1.19 (t, J = 7.5 Hz, 3H). |
| 189 | 10.34 (s, 1H), 8.06 (d, J = 8.5 Hz, 1H), 7.97 (s, 1H), 7.73 (d, J = 8.8 Hz, 1H), 7.36-7.31 (m, 2H), 7.25 (d, |
| | J = 6.9 Hz, 3H), 6.83 (s, 1H), 6.50 (s, 1H), 5.32 (s, 2H), 4.45 (s, 2H), 4.34 (s, 2H), 4.26 (d, J = 2.2 Hz, |
| | 2H), 3.96 (d, J = 5.4 Hz, 4H), 3.80 (t, J = 5.3 Hz, 2H), 3.47 (s, 2H), 3.29-3.15 (m, 2H), 2.99 (d, J = 7.5 |
| | Hz, 2H), 2.80 (s, 3H), 2.71 (d, J = 10.2 Hz, 2H), 1.20(t, J = 7.4 Hz, 3H). |
| 190 | 10.34 (s, 1H), 8.06 (d, J = 8.5 Hz, 1H), 7.97 (d, J = 1.5 Hz, 1H), 7.72 (dd, J = 8.7, 1.6 Hz, 1H), 6.83 (s, |
| | 1H), 6.35 (t, J = 5.6 Hz, 1H), 6.00 (t, J = 4.9 Hz, 1H), 5.32 (s, 2H), 4.38 (d, J = 12.3 Hz, 1H), 4.25 (d, |
| | J = 2.4 Hz, 2H), 4.04-3.84 (m, 3H), 3.80 (t, J = 5.4 Hz, 3H), 3.46 (t, J = 10.7 Hz, 1H), 3.38 (d, J = 12.9 Hz, |
| | 1H), 3.19 (t, J = 11.4 Hz, 1H), 2.98 (q, J = 7.1 Hz, 2H), 2.89 (t, J = 6.2 Hz, 2H), 2.79 (d, J = 13.5 Hz, |
| | 1H), 2.70 (d, J = 10.8 Hz, 3H), 1.19 (t, J = 7.5 Hz, 3H), 0.91-0.81 (m, 1H), 0.41-0.35 (m, 2H), 0.15-0.10 |
| | (m, 2H). |
| 191 | 10.36 (s, 1H), 8.06 (d, J = 8.3 Hz, 1H), 7.97 (d, J = 1.5 Hz, 2H), 7.72 (dd, J = 8.7, 1.5 Hz, 1H), 7.37-7.25 |
| | (m, 2H), 7.16 (ddd, J = 13.6, 7.1, 4.9 Hz, 1H), 6.83 (s, 1H), 6.78 (t, J = 6.0 Hz, 1H), 6.18 (t, J = 5.0 Hz, |
| | 1H), 5.32 (s, 2H), 4.38 (d, J = 11.4 Hz, 1H), 4.26 (d, J = 6.2 Hz, 5H), 3.97 (dt, J = 17.2, 12.7 Hz, 2H), |
| | 3.80 (t, J = 5.4 Hz, 3H), 3.46 (t, J = 10.7 Hz, 1H), 3.38 (d, J = 10.9 Hz, 1H), 3.19 (t, J = 11.5 Hz, 1H), |
| | 2.98 (d, J = 7.4 Hz, 2H), 2.84-2.64 (m, 4H), 1.19 (t, J = 7.4 Hz, 3H). |
| 192 | 10.37 (s, 1H), 8.60 (s, 1H), 8.49 (d, J = 5.0 Hz, 1H), 8.07 (d, J = 8.5 Hz, 1H), 7.98 (d, J = 1.5 Hz, 1H), |
| | 7.73 (d, J = 8.6 Hz, 1H), 7.43 (d, J = 5.2 Hz, 1H), 6.84 (s, 1H), 6.52 (t, J = 4.7 Hz, 1H), 5.33 (s, 2H), |
| | 4.68 (d, J = 6.4 Hz, 4H), 4.40 (d, J = 11.6 Hz, 1H), 4.26 (d, J = 2.3 Hz, 2H), 4.00 (d, J = 3.6 Hz, 2H), |
| | 3.93 (d, J = 11.6 Hz, 1H), 3.81 (t, J = 5.4 Hz, 2H), 3.49 (s, 1H), 3.39 (s, 2H), 3.25 (d, J = 12.8 Hz, 2H), |
| | 3.00 (d, J = 7.7 Hz, 2H), 2.81 (d, J = 12.3 Hz, 1H), 2.72 (d, J = 11.5 Hz, 2H), 1.20 (t, J = 7.5 Hz, 3H). |
| 193 | 10.36 (s, 1H), 8.06 (d, J = 8.5 Hz, 1H), 7.97 (s, 1H), 7.72 (d, J = 7.1 Hz, 1H), 7.07-7.03 (m, 1H), 7.02- |
| | 6.97 (m, 2H), 6.96 (d, J = 4.6 Hz, 1H), 6.83 (s, 1H), 5.32 (s, 2H), 4.37 (s, 1H), 4.25 (d, J = 2.4 Hz, 2H), |
| | 4.14-4.01 (m, 4H), 3.90 (d, J = 12.5 Hz, 1H), 3.80 (t, J = 5.4 Hz, 2H), 3.43 (d, J = 45.5 Hz, 2H), 3.26 |
| | (d, J = 30.1 Hz, 3H), 3.10 (t, J = 8.3 Hz, 2H), 2.99 (d, J = 7.7 Hz, 2H), 2.79 (s, 1H), 2.71 (d, J = 11.2 Hz, |
| | 2H), 1.19 (t, J = 7.5 Hz, 3H). |

### Example 194: N-((4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl) sulfonyl)benzamide

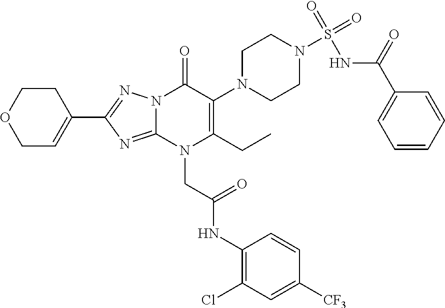 [SMILES: CCc1c(N2CCN(S(=O)(=O)NC(=O)c3ccccc3)CC2)c(=O)n2nc(C3=CCOCC3)nc2n1CC(=O)Nc1ccc(C(F)(F)F)cc1Cl]

### Step 1: Benzoylsulfamoyl Chloride

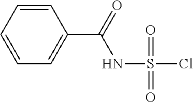 [SMILES: O=C(NS(=O)(=O)Cl)c1ccccc1]

To a solution of benzoic acid (200 mg, 1.64 mmol) in CHCl₃ (3 mL) was added sulfurisocyanatidic chloride (344.4 mg, 2.46 mmol) at r.t. The mixture was stirred at 50° C. for 2 h., quenched with H₂O (5 mL) and extracted with DCM (5 mL×3). The combined organic layers were concentrated. The residue was recrystallized from acetone (10 mL) to afford the title compound (120 mg, 33% yield) as a white solid.

### Step 2: N-((4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl) sulfonyl)benzamide

To a solution of INT B1 (30 mg, 0.05 mmol), benzoylsulfamoyl chloride (17.4 mg, 0.08 mmol) in DCM (1 mL) was added TEA (20.5 mg, 0.16 mmol) at r.t. The mixture was stirred at r.t. for 2 h., quenched with H₂O (5 mL) and extracted with DCM (5 mL×3). The organic layers were washed with saturated NH₄Cl aq. (5 mL) and brine (5 mL), dried over anhydrous Na₂SO₄, filtered and concentrated. The residue was purified by Prep-HPLC on a C18 column eluting with MeCN/H₂O (5-95%, with 0.1% NH₄HCO₃) to afford the title compound (10.4 mg, 26% yield) as a white solid. ¹H NMR (400 MHz, DMSO-d₆) δ ppm 11.96 (s, 1H), 10.35 (s, 1H), 8.05 (d, J=8.6 Hz, 1H), 7.98-7.92 (m, 3H), 7.71 (d, J=6.5 Hz, 1H), 7.61 (s, 1H), 7.51 (t, J=7.4 Hz, 2H), 6.83 (s, 1H), 5.31 (s, 2H), 4.25 (d, J=2.6 Hz, 2H), 3.81 (t, J=5.5 Hz, 2H), 3.73 (d, J=10.5 Hz, 2H), 3.56 (t, J=10.5 Hz, 3H), 3.08 (d, J=10.1 Hz, 2H), 2.93 (d, J=7.5 Hz, 2H), 2.75 (d, J=11.6 Hz, 3H), 1.15 (t, J=7.6 Hz, 3H). LCMS calc. for C₃₂H₃₃ClF₃N₈O₆S [M+H]⁺: m/z=749.2; Found: 749.8.

### Example 195: N-(2-Chloro-4-(trifluoromethyl)phenyl)-2-(2-(3,6-dihydro-2H-pyran-4-yl)-5-ethyl-6-(4-(7-hydroxythiazolo[4,5-c]pyridine-6-carbonyl)piperazin-1-yl)-7-oxo-[1,2,4]triazolo[1,5-a]pyrimidin-4(7H)-yl)acetamide

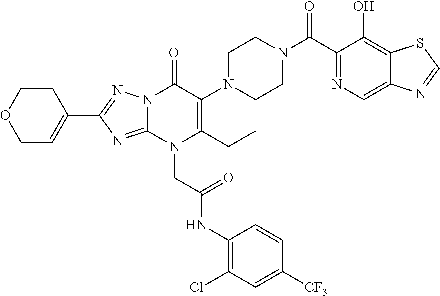 [SMILES: CCc1c(N2CCN(C(=O)c3ncc4ncsc4c3O)CC2)c(=O)n2nc(C3=CCOCC3)nc2n1CC(=O)Nc1ccc(C(F)(F)F)cc1Cl]

Method G: To a mixture of INT C40 (18 mg, 0.08 mmol), INT B1 (35 mg, 0.06 mmol) was added a solution of T₃P in DMF (50%, 0.3 mL) and TEA (0.3 mL). The mixture was stirred at r.t. for 12 h., and concentrated. The residue was purified by Prep-HPLC on a C18 column eluting with MeCN/H₂O (45%-70%, with 0.05% FA) to afford the title compound (3 mg, 5% yield) as a white solid. ¹H NMR (400 MHZ, DMSO-d₆) δ ppm 12.58 (s, 1H), 10.36 (s, 1H), 9.32 (s, 1H), 8.29 (s, 1H), 8.06 (d, J=8.6 Hz, 1H), 7.96 (d, J=1.7 Hz, 1H), 7.71 (dd, J=8.8, 1.7 Hz, 1H), 6.86-6.80 (m, 1H), 5.32 (s, 2H), 4.57 (s, 1H), 4.30-4.22 (m, 2H), 3.80 (t, J=5.4 Hz, 2H), 3.58-3.39 (m, 4H), 2.99 (d, J=7.8 Hz, 3H), 2.90-2.71 (m, 2H), 2.70-2.54 (m, 2H), 1.19 (t, J=7.4 Hz, 3H). LCMS calc. for C₃₂H₃₀ClF₃N₉O₅S [M+H]⁺: m/z=744.2; Found: 744.4.

### Example 196: N-(2-Chloro-4-(trifluoromethyl)phenyl)-2-(6-(4-(2-chloro-7-hydroxythiazolo[4,5-c]pyridine-6-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)acetamide

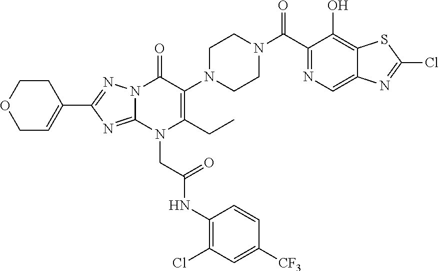 [SMILES: CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1Cl]

This compound was prepared by procedures analogous to those described for Method G using INT C41 to replace INT C40 to afford the title product as a white solid. ¹H NMR (400 MHZ, DMSO-d₆) δ ppm 11.94 (s, 1H), 10.37 (s, 1H), 8.64 (s, 1H), 8.06 (d, J=8.5 Hz, 1H), 7.97 (d, J=2.1 Hz, 1H), 7.72 (dd, J=8.8, 2.1 Hz, 1H), 6.83 (s, 1H), 5.32 (s, 2H), 4.58 (d, J=12.7 Hz, 1H), 4.25 (d, J=3.2 Hz, 2H), 3.80 (t, J=5.4 Hz, 2H), 3.70 (s, 1H), 3.51 (d, J=13.4 Hz, 2H), 3.25 (d, J=11.2 Hz, 3H), 2.99 (d, J=7.7 Hz, 3H), 2.82 (d, J=11.7 Hz, 1H), 2.62 (d, J=10.5 Hz, 1H), 1.19 (t, J=7.4 Hz, 3H). LCMS calc. for C₃₂H₂₉C₁₂F₃N₉O₅S [M+H]⁺: m/z=778.1; Found: 778.4.

### Example 197: N-(2-Chloro-4-(trifluoromethyl)phenyl)-2-(2-(3,6-dihydro-2H-pyran-4-yl)-5-ethyl-7-oxo-6-(4-(5-oxo-4,5-dihydrothieno[3,2-b]pyridine-6-carbonyl)piperazin-1-yl)-[1,2,4]triazolo[1,5-a]pyrimidin-4(7H)-yl)acetamide

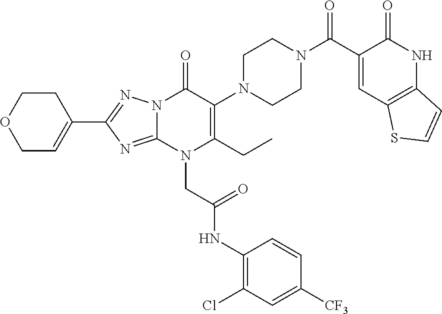 [SMILES: CCc1c(N2CCN(C(=O)c3cc4sccc4[nH]c3=O)CC2)c(=O)n2nc(C3=CCOCC3)nc2n1CC(=O)Nc1ccc(C(F)(F)F)cc1Cl]

This compound was prepared by procedures analogous to those described for method A using INT C52 to replace 4-(trifluoromethyl)-1H-imidazole-2-carboxylic acid to afford the title compound as a yellow solid. ¹H NMR (400 MHZ, DMSO-d₆)₈ ppm 12.43 (s, 1H), 10.36 (s, 1H), 8.17 (s, 1H), 8.06 (d, J=8.6 Hz, 1H), 8.00-7.92 (m, 2H), 7.71 (dd, J=8.8, 2.2 Hz, 1H), 7.02 (d, J=5.4 Hz, 1H), 6.83 (s, 1H), 5.32 (s, 2H), 4.51 (d, J=12.5 Hz, 1H), 4.25 (d, J=3.1 Hz, 2H), 3.80 (t, J=5.4 Hz, 2H), 3.48 (q, J=11.5, 11.0 Hz, 4H), 3.25 (d, J=11.0 Hz, 2H), 3.05-2.87 (m, 3H), 2.78 (d, J=10.7 Hz, 1H), 2.63 (d, J=10.7 Hz, 1H), 1.19 (t, J=7.4 Hz, 3H). LCMS calc. for C₃₃H₃₁ClF₃N₈O₅S [M+H]⁺: m/z=743.2; Found: 743.1.

### Example 198:2-(6-(4-(7-Chloro-2-hydroxypyrazolo[1,5-a]pyridine-3-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

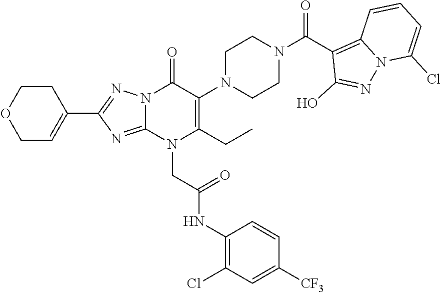 [SMILES: CCc1c(N2CCN(C(=O)c3c(O)nn4c(Cl)cccc34)CC2)c(=O)n2nc(C3=CCOCC3)nc2n1CC(=O)Nc1ccc(C(F)(F)F)cc1Cl]

This compound was prepared by procedures analogous to those described for method A using INT C53 to replace 4-(trifluoromethyl)-1H-imidazole-2-carboxylic acid to afford the title compound as a white solid. ¹H NMR (400 MHZ, DMSO-d₆)₈ ppm 11.94 (s, 1H), 10.37 (s, 1H), 8.06 (d, J=8.6 Hz, 1H), 7.97 (d, J=1.6 Hz, 1H), 7.72 (dd, J=8.8, 1.7 Hz, 1H), 7.63 (d, J=9.6 Hz, 1H), 7.35 (dd, J=8.7, 7.6 Hz, 1H), 7.16 (d, J=8.2 Hz, 1H), 6.83 (s, 1H), 5.32 (s, 2H), 4.25 (d, J=2.3 Hz, 2H), 4.18 (s, 1H), 3.80 (t, J=5.4 Hz, 3H), 3.52-3.49 (m, 4H), 3.13 (s, 2H), 3.01 (d, J=7.4 Hz, 2H), 2.73 (d, J=10.6 Hz, 2H), 1.20 (t, J=7.4 Hz, 3H). LCMS calc. for C₃₃H₃₁Cl₂F₃N₉O₅ [M+H]⁺: m/z=760.2; Found: 760.0.

### Example 199:2-(6-(4-(2-Chloro-7-hydroxythiazolo[4,5-c]pyridine-6-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-fluoro-4-(trifluoromethyl)phenyl)acetamide

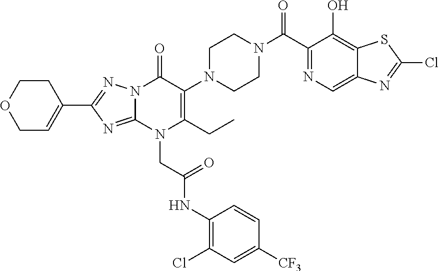 [SMILES: CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1Cl]

To a mixture of INT C41 (20.7 mg, 0.09 mmol), INT B13 (49.4 mg, 0.09 mmol) in pyridine (0.5 mL) was added EDCI (34.5 mg, 0.18 mmol). The mixture was stirred at r.t. overnight. The reaction mixture was purified by Prep-HPLC eluting with MeCN/H₂O (5-55%) to afford the title compound (8.9 mg, 12% yield) as a white solid. LCMS calc. for C₃₂H₂₇ClF₄N₉O₅S [M−H]⁻: m/z=760.2; Found: 760.1.

### Example 200: N-(2-Chloro-4-(trifluoromethyl)phenyl)-2-(2-(3,6-dihydro-2H-pyran-4-yl)-5-ethyl-6-(4-(4-hydroxypyrazolo[1,5-a]pyridine-5-carbonyl)piperazin-1-yl)-7-oxo-[1,2,4]triazolo[1,5-a]pyrimidin-4(7H)-yl)acetamide

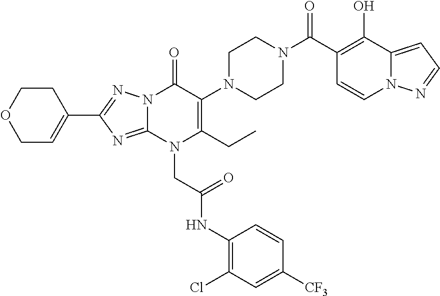 [SMILES: CCc1c(N2CCN(C(=O)c3ccn4nccc4c3O)CC2)c(=O)n2nc(C3=CCOCC3)nc2n1CC(=O)Nc1ccc(C(F)(F)F)cc1Cl]

This compound was prepared by procedures analogous to those described for method A using INT C45 and INT B1 to afford the title compound as a white solid. ¹H NMR (400 MHZ, DMSO-d₆) δ ppm 10.68 (s, 1H), 10.36 (s, 1H), 8.27 (d, J=7.2 Hz, 1H), 8.06 (d, J=8.8 Hz, 1H), 7.96 (d, J=1.6 Hz, 1H), 7.94 (d, J=2.4 Hz, 1H), 7.72 (dd, 1H), 6.91 (dd, J=2.4, 0.8 Hz, 1H), 6.87-6.79 (m, 1H), 6.70 (d, J=7.2 Hz, 1H), 5.32 (s, 2H), 4.25 (d, J=2.4 Hz, 2H), 4.07 (s, 2H), 3.80 (t, J=5.2 Hz, 2H), 3.57-3.45 (m, 2H), 3.30 (s, 2H), 3.13 (t, J=11.2 Hz, 2H), 2.99 (q, J=7.2 Hz, 2H), 2.73 (d, J=10.8 Hz, 2H), 1.19 (t, J=7.6 Hz, 3H). LCMS calc. for C₃₃H₃₂ClF₃N₉O₅ [M+H]⁺: m/z=726.2; Found: 726.2.

### Example 201: N-(2-Chloro-4-(trifluoromethyl)phenyl)-2-(2-(3,6-dihydro-2H-pyran-4-yl)-5-ethyl-6-(4-(4-hydroxyimidazo[1,5-b]pyridazine-3-carbonyl)piperazin-1-yl)-7-oxo-[1,2,4]triazolo[1,5-a]pyrimidin-4(7H)-yl)acetamide

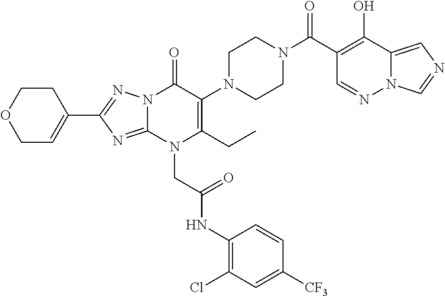 [SMILES: CCc1c(N2CCN(C(=O)c3cnn4cncc4c3O)CC2)c(=O)n2nc(C3=CCOCC3)nc2n1CC(=O)Nc1ccc(C(F)(F)F)cc1Cl]

This compound was prepared by procedures analogous to those described for method A using INT C47 and INT B1 to afford the title compound as a white solid. ¹H NMR (400 MHZ, DMSO-d₆) d ppm 10.36 (s, 1H), 9.01 (s, 1H), 8.09-8.02 (m, 2H), 7.97 (d, J=1.6 Hz, 1H), 7.86 (s, 1H), 7.72 (dd, J=8.8, 1.6 Hz, 1H), 6.83 (s, 1H), 5.32 (s, 2H), 4.25 (d, J=2.4 Hz, 2H), 4.09 (s, 2H), 3.80 (t, J=5.2 Hz, 2H), 3.53-3.45 (m, 2H), 3.29-3.21 (m, 2H), 3.17-3.05 (m, 2H), 3.04-2.96 (m, 2H), 2.74-2.66 (m, 2H), 1.20 (t, J=7.2 Hz, 3H). LCMS calc. for C₃₂H₃₁ClF₃N₁₀O₅ [M+H]⁺: m/z=727.2; Found: 727.4.

### Example 202: N-(2-Chloro-4-(trifluoromethyl)phenyl)-2-(2-(3,6-dihydro-2H-pyran-4-yl)-5-ethyl-6-(4-(4-hydroxypyrrolo[1,2-c]pyrimidine-3-carbonyl)piperazin-1-yl)-7-oxo-[1,2,4]triazolo[1,5-a]pyrimidin-4(7H)-yl)acetamide

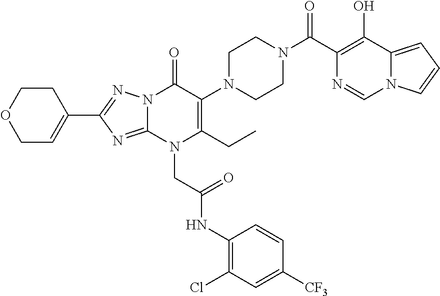 [SMILES: CCc1c(N2CCN(C(=O)c3ncn4cccc4c3O)CC2)c(=O)n2nc(C3=CCOCC3)nc2n1CC(=O)Nc1ccc(C(F)(F)F)cc1Cl]

This compound was prepared by procedures analogous to those described for method A using INT C56 and INT B1 to afford the title compound as a white solid. ¹H NMR (400 MHZ, DMSO-d₆) 0 ppm 11.66 (s, 1H), 10.37 (s, 1H), 8.41 (s, 1H), 8.06 (d, J=8.6 Hz, 1H), 7.97 (s, 1H), 7.72 (dd, J=8.6, 1.3 Hz, 1H), 7.07 (s, 1H), 6.83 (s, 1H), 6.74-6.69 (m, 1H), 6.25 (dd, J=5.9, 2.5 Hz, 1H), 5.33 (s, 2H), 4.69 (d, J=11.0 Hz, 1H), 4.58 (d, J=14.5 Hz, 1H), 4.25 (s, 2H), 3.80 (t, J=5.3 Hz, 4H), 3.52 (s, 4H), 3.04-2.99 (m, 2H), 2.83 (d, J=8.4 Hz, 1H), 2.75 (d, J=9.9 Hz, 1H), 1.21 (t, J=7.4 Hz, 3H). LCMS calc. for C₃₃H₃₂ClF₃N₉O₅ [M+H]⁺: m/z=726.2; Found: 726.4.

### Example 203: N-(2-Chloro-4-(trifluoromethyl)phenyl)-2-(2-(3,6-dihydro-2H-pyran-4-yl)-5-ethyl-6-(4-(7-hydroxy-1-methyl-1H-pyrrolo[3,2-c]pyridine-6-carbonyl)piperazin-1-yl)-7-oxo-[1,2,4]triazolo[1,5-a]pyrimidin-4(7H)-yl)acetamide

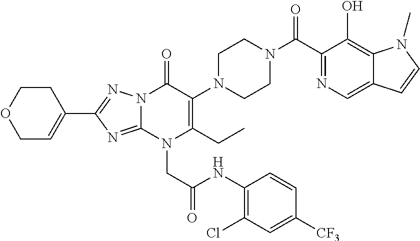 [SMILES: CCc1c(N2CCN(C(=O)c3ncc4ccn(C)c4c3O)CC2)c(=O)n2nc(C3=CCOCC3)nc2n1CC(=O)Nc1ccc(C(F)(F)F)cc1Cl]

Method H: To a solution of INT C42 (12 mg, 0.06 mmol) in MeCN (1 mL) was added TCFH (28 mg, 0.10 mmol), NMI (14 mg, 0.18 mmol), INT B1 (30 mg, 0.05 mmol). The mixture was stirred at r.t. for 16 h. The reaction mixture was filtered and concentrated. The residue was purified by Prep-HPLC on a C18 column eluting with MeCN/H₂O (30-50%, with 0.1% FA) to afford the title compound (23 mg, 62% yield) as a white solid. ¹H NMR (400 MHZ, DMSO-d₆) δ ppm 10.38 (s, 1H), 8.43 (s, 1H), 8.06 (d, J=8.5 Hz, 1H), 7.97 (s, 1H), 7.72 (d, J=8.6 Hz, 1H), 7.54 (s, 1H), 6.83 (s, 1H), 6.64 (s, 1H), 5.33 (s, 2H), 4.26 (s, 2H), 4.12 (s, 3H), 3.81 (d, J=5.4 Hz, 2H), 3.60-3.52 (m, 9H), 3.02 (dd, J=14.4, 7.0 Hz, 2H), 2.80 (dd, J=12.5, 6.5 Hz, 2H), 1.23 (d, J=7.2 Hz, 3H). LCMS calc. for C₃₄H₃₄ClF₃N₉O₅ [M+H]⁺: m/z=740.2; Found: 740.2.

### Example 204: N-(2-Chloro-4-(trifluoromethyl)phenyl)-2-(2-(3,6-dihydro-2H-pyran-4-yl)-5-ethyl-6-(4-(4-hydroxyisoquinoline-3-carbonyl)piperazin-1-yl)-7-oxo-[1,2,4]triazolo[1,5-a]pyrimidin-4(7H)-yl)acetamide

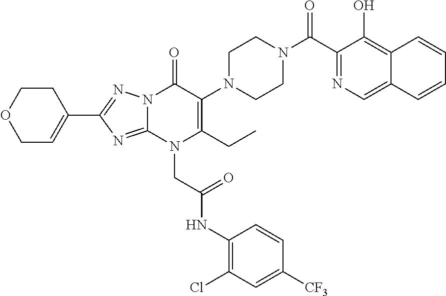 [SMILES: CCc1c(N2CCN(C(=O)c3ncc4ccccc4c3O)CC2)c(=O)n2nc(C3=CCOCC3)nc2n1CC(=O)Nc1ccc(C(F)(F)F)cc1Cl]

This compound was prepared by procedures analogous to those described for Method H using INT C50 to replace INT C42 to afford the title compound as a white solid. ¹H NMR (400 MHZ, DMSO-d₆)₈ ppm 12.59 (s, 1H), 10.37 (s, 1H), 8.85 (s, 1H), 8.30 (d, J=8.0 Hz, 1H), 8.14 (d, J=7.4 Hz, 1H), 8.07 (d, J=8.5 Hz, 1H), 7.97 (d, J=1.6 Hz, 1H), 7.88-7.79 (m, 2H), 7.73 (d, J=8.8 Hz, 1H), 6.83 (s, 1H), 5.33 (s, 2H), 4.83 (d, J=66.2 Hz, 2H), 4.25 (d, J=2.4 Hz, 2H), 3.80 (t, J=5.4 Hz, 2H), 3.59 (t, J=10.5 Hz, 2H), 3.33 (s, 2H), 3.02 (dd, J=14.5, 7.1 Hz, 2H), 2.81 (s, 2H), 2.52 (s, 2H), 1.22 (t, J=7.4 Hz, 3H). LCMS calc. for C₃₅H₃₃ClF₃N₈O₅ [M+H]⁺: m/z=737.2; Found: 737.0. Example 205: N-(2-Chloro-4-(trifluoromethyl)phenyl)-2-(2-(3,6-dihydro-2H-pyran-4-yl)-5-ethyl-6-(4-(4-hydroxy-1-methyl-1H-pyrrolo[2,3-c]pyridine-5-carbonyl)piperazin-1-yl)-7-oxo-[1,2,4]triazolo[1,5-a]pyrimidin-4(7H)-yl)acetamide

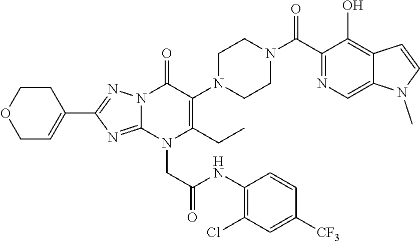 [SMILES: CCc1c(N2CCN(C(=O)c3ncc4c(ccn4C)c3O)CC2)c(=O)n2nc(C3=CCOCC3)nc2n1CC(=O)Nc1ccc(C(F)(F)F)cc1Cl]

This compound was prepared by procedures analogous to those described for Method H using INT C44 to replace INT C42 to afford the title compound as a white solid. ¹H NMR (400 MHZ, DMSO-d₆)₈ ppm 12.06 (s, 1H), 10.37 (s, 1H), 8.42 (s, 1H), 8.06 (d, J=8.6 Hz, 1H), 7.97 (d, J=1.5 Hz, 1H), 7.72 (d, J=7.1 Hz, 1H), 7.49 (d, J=3.0 Hz, 1H), 6.83 (s, 1H), 6.68 (d, J=2.6 Hz, 1H), 5.33 (s, 2H), 4.25 (d, J=2.5 Hz, 2H), 3.90 (s, 3H), 3.80 (t, J=5.4 Hz, 2H), 3.55 (t, J=10.5 Hz, 2H), 3.30 (s, 6H), 3.02 (d, J=7.3 Hz, 2H), 2.76 (s, 2H), 1.22 (d, J=7.6 Hz, 3H). LCMS calc. for C₃₄H₃₄ClF₃N₉O₅ [M+H]⁺: m/z=740.2; Found: 740.2.

### Example 206: N-(2-Chloro-4-(trifluoromethyl)phenyl)-2-(2-(3,6-dihydro-2H-pyran-4-yl)-5-ethyl-6-(4-(8-hydroxyimidazo[1,2-b]pyridazine-7-carbonyl)piperazin-1-yl)-7-oxo-[1,2,4]triazolo[1,5-a]pyrimidin-4(7H)-yl)acetamide

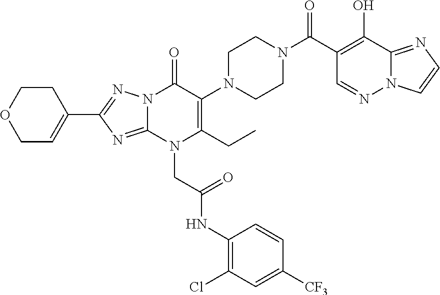 [SMILES: CCc1c(N2CCN(C(=O)c3cnn4ccnc4c3O)CC2)c(=O)n2nc(C3=CCOCC3)nc2n1CC(=O)Nc1ccc(C(F)(F)F)cc1Cl]

This compound was prepared by procedures analogous to those described for Method H using INT C54 to replace INT C42 to afford the title compound as a white solid. ¹H NMR (400 MHZ, DMSO-d₆)₈ ppm 10.36 (s, 1H), 8.06 (d, J=8.7 Hz, 2H), 7.98 (d, J=7.9 Hz, 2H), 7.71 (d, J=8.6 Hz, 2H), 6.83 (s, 1H), 5.32 (s, 2H), 4.61-4.37 (m, 1H), 4.25 (s, 3H), 3.80 (t, J=5.3 Hz, 4H), 3.49 (s, 4H), 3.00 (d, J=7.5 Hz, 3H), 2.67 (s, 1H), 1.20 (t, J=7.4 Hz, 3H). LCMS calc. for C₃₂H₃₁ClF₃N₁₀O₅ [M+H]⁺: m/z=727.2; Found: 727.0.

### Example 207: N-(2-Chloro-4-(trifluoromethyl)phenyl)-2-(2-(3,6-dihydro-2H-pyran-4-yl)-5-ethyl-7-oxo-6-(4-(pyrimidin-2-ylglycyl)piperazin-1-yl)-[1,2,4]triazolo[1,5-a]pyrimidin-4(7H)-yl)acetamide

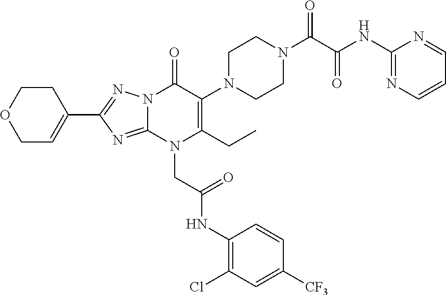 [SMILES: CCc1c(N2CCN(C(=O)C(=O)Nc3ncccn3)CC2)c(=O)n2nc(C3=CCOCC3)nc2n1CC(=O)Nc1ccc(C(F)(F)F)cc1Cl]

A solution of 2-(methylsulfonyl)pyrimidine (9 mg, 0.06 mmol), N-(2-chloro-4-(trifluoromethyl)phenyl)-2-(2-(3,6-dihydro-2H-pyran-4-yl)-5-ethyl-6-(4-glycyl piperazin-1-yl)-7-oxo-[1,2,4]triazolo[1,5-a]pyrimidin-4(7H)-yl)acetamide (30 mg, 0.05 mmol, Example 38) and DIPEA (58 mg, 0.45 mmol) in DMSO (1 mL) was stirred at 90° C. for 16 h. The reaction mixture was concentrated under reduced pressure. The residue was purified by Prep-HPLC on a C18 column eluting with MeCN/H₂O (45-75%, with 0.5% FA) to afford the title compound (12.7 mg, 36% yield) as a white solid. ¹H NMR (400 MHZ, DMSO-d₆) δ ppm 10.38 (s, 1H), 8.30 (d, J=4.7 Hz, 2H), 8.06 (d, J=8.6 Hz, 1H), 7.97 (s, 1H), 7.72 (d, J=7.3 Hz, 1H), 6.97 (t, J=5.8 Hz, 1H), 6.84 (s, 1H), 6.62 (t, J=4.8 Hz, 1H), 5.32 (s, 2H), 4.37 (d, J=12.5 Hz, 1H), 4.26 (s, 2H), 4.18 (dd, J=16.4, 5.7 Hz, 2H), 3.94 (d, J=12.9 Hz, 2H), 3.81 (d, J=5.3 Hz, 2H), 3.52 (s, 2H), 3.29-3.22 (m, 2H), 2.99 (dd, J=15.1, 7.6 Hz, 2H), 2.80 (d, J=12.0 Hz, 1H), 2.72 (d, J=11.3 Hz, 2H), 1.20 (t, J=7.4 Hz, 3H). LCMS calc. for C₃₁H₃₃ClF₃N₁₀O₄ [M+H]⁺: m/z=701.2; Found: 701.2.

### Example 208: N-(2-Chloro-4-(trifluoromethyl)phenyl)-2-(2-(3,6-dihydro-2H-pyran-4-yl)-5-ethyl-7-oxo-6-(4-((4-oxo-1,4-dihydroquinazolin-2-yl)glycyl)piperazin-1-yl)-[1,2,4]triazolo[1,5-a]pyrimidin-4(7H)-yl)acetamide

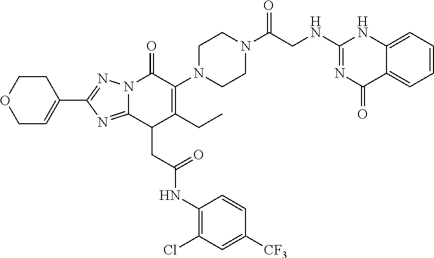 [SMILES: CCC1=C(N2CCN(C(=O)CNc3nc(=O)c4ccccc4[nH]3)CC2)C(=O)n2nc(C3=CCOCC3)nc2C1CC(=O)Nc1ccc(C(F)(F)F)cc1Cl]

To a solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 48.2 μmol, Example 38) in EtOH (2 mL) was added 2-chloroquinazolin-4(1H)-one (10.4 mg, 57.9 μmol) and DIPEA (18.66 mg, 144.7 μmol). The reaction mixture was degassed with N₂, and irradiated in microwave at 130° C. for 2 h. The reaction mixture was quenched with H₂O (10 mL) and extracted with DCM (15 mL×3). The combined organic layers were washed with NH₄Cl aq. and brine, dried over Na₂SO₄, filtered and concentrated. The residue was purified by Prep-HPLC on a C18 column eluting with MeCN/H₂O (5-95%, with 0.1% NH₄HCO₃) to afford the title product (3.63 mg, 9.8% yield) as a white solid. ¹H NMR (400 MHZ, DMSO-d₆) δ ppm 11.22 (s, 1H), 10.38 (s, 1H), 8.07 (d, J=8.4 Hz, 1H), 7.98 (d, J=1.6 Hz, 1H), 7.90 (dd, J=8.0, 1.2 Hz, 1H), 7.73 (d, J=7.2 Hz, 1H), 7.28 (d, J=8.0 Hz, 1H), 7.15-7.08 (m, 1H), 6.85-6.83 (m, 1H), 6.68-6.67 (m, 1H), 5.33 (s, 2H), 4.43-4.42 (m, 1H), 4.32-4.30 (m, 1H), 4.26-4.24 (m, 3H), 3.87-3.85 (m, 1H), 3.81-3.79 (m, 2H), 3.53-3.51 (m, 1H), 3.39-3.37 (m, 1H), 3.32-3.30 (m, 2H), 3.29-3.27 (m, 2H), 3.02-3.00 (m, 2H), 2.86-2.84 (m, 1H), 2.78-2.75 (m, 2H), 1.21 (t, J=7.6 Hz, 3H). LCMS calc. for C₃₅H₃₅ClF₃N₁₀O₅ [M+H]⁺: m/z=767.2; Found: 767.4.

### Example 209: N-(2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl-1,1-d₂)-2-fluoro-5-(methylamino)benzamide

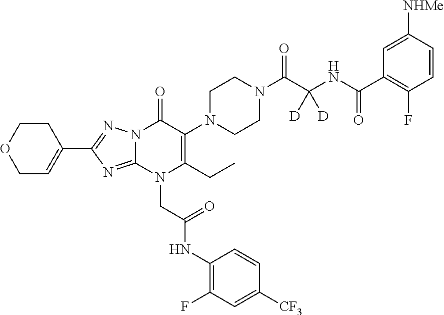 [SMILES: [2H]C([2H])(NC(=O)c1cc(NC)ccc1F)C(=O)N1CCN(c2c(CC)n(CC(=O)Nc3ccc(C(F)(F)F)cc3F)c3nc(C4=CCOCC4)nn3c2=O)CC1]

### Step 1: tert-butyl (2-(4-(2-(3,6-dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl-1,1-d₂)carbamate

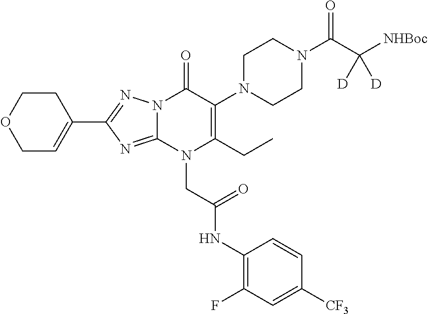 [SMILES: [2H]C([2H])(NC(=O)OC(C)(C)C)C(=O)N1CCN(c2c(CC)n(CC(=O)Nc3ccc(C(F)(F)F)cc3F)c3nc(C4=CCOCC4)nn3c2=O)CC1]

A mixture of INT C57 (53 mg, 0.3 mmol), INT B13 (110 mg, 0.2 mmol), EDCI (76 mg, 0.4 mmol), HOBt (50 mg, 0.4 mmol) and TEA (101 mg, 1 mmol) in DMF (1 mL) was stirred at r.t. overnight. The mixture was diluted with water (10 mL). The precipitate was collected by filtration to afford the title compound (130 mg) as a white solid. LCMS calc. for C₃₂H₃₅D₂F₄N₈O₆ [M−H]⁻: m/z=707.3; Found: 707.2.

### Step 2: 2-(2-(3,6-dihydro-2H-pyran-4-yl)-5-ethyl-6-(4-(glycyl-2,2-d₂)piperazin-1-yl)-7-oxo-[1,2,4]triazolo[1,5-a]pyrimidin-4(7H)-yl)-N-(2-fluoro-4-(trifluoromethyl)phenyl)acetamide

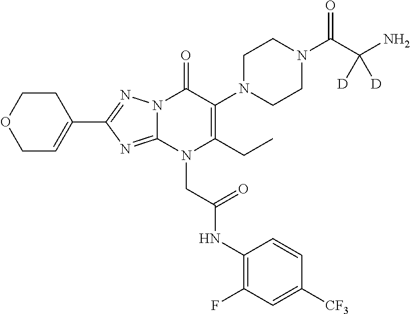 [SMILES: [2H]C([2H])(N)C(=O)N1CCN(c2c(CC)n(CC(=O)Nc3ccc(C(F)(F)F)cc3F)c3nc(C4=CCOCC4)nn3c2=O)CC1]

A mixture of tert-butyl (2-(4-(2-(3,6-dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl-1,1-d₂)carbamate (130 mg, 0.18 mmol) in DCM (3 mL) and 4 M HCl/dioxane (1 mL) was stirred at r.t. for 2 h. The precipitate was collected by filtration to afford the title compound (100 mg) as a brown solid. LCMS calc. for C₂₇H₂₉D₂F₄N₈O₄ [M+H]⁺: m/z=609.3; Found: 609.2.

### Step 3: N-(2-(4-(2-(3,6-dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl-1,1-d₂)-2-fluoro-5-(methylamino)benzamide

A mixture of 2-fluoro-5-(methylamino)benzoic acid (34 mg, 0.2 mmol), 2-(2-(3,6-dihydro-2H-pyran-4-yl)-5-ethyl-6-(4-(glycyl-2,2-d₂)piperazin-1-yl)-7-oxo-[1,2,4]triazolo[1,5-a]pyrimidin-4(7H)-yl)-N-(2-fluoro-4-(trifluoromethyl)phenyl)acetamide (100 mg, 0.16 mmol), EDCI (57 mg, 0.3 mmol), HOBt (45 mg, 0.3 mmol) and TEA (101 mg, 1.0 mmol) in DMF (1 mL) was stirred at r.t. overnight. The mixture was concentrated under reduced pressure. The residue was purified by flash chromatography on a C18 column eluting with MeCN/H₂O (40-55% with 0.1% NH₄HCO₃) to afford the title compound (25.3 mg) as a white solid. LCMS calc. for C₃₅H₃₃D₂F₅N₉O₅ [M−H]⁻: m/z=758.3; Found: 758.2.

### Example 210: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-hydroxy-6-methylpyrimidine-4-carboxamide

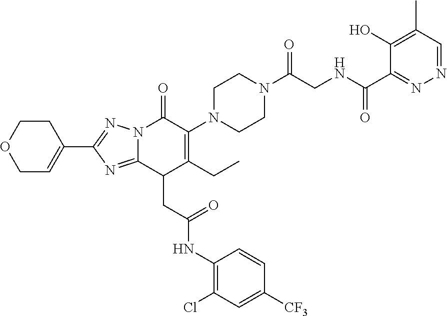 [SMILES: CCC1=C(N2CCN(C(=O)CNC(=O)c3nncc(C)c3O)CC2)C(=O)n2nc(C3=CCOCC3)nc2C1CC(=O)Nc1ccc(C(F)(F)F)cc1Cl]

Method I: A mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 5-hydroxy-6-methylpyrimidine-4-carboxylic acid (INT C81, 9 mg, 0.06 mmol), EDCI (12 mg, 0.07 mmol), HOBt (8 mg, 0.06 mmol) and DIEA (25 mg, 0.19 mmol) in DMF (2 mL) was stirred at r.t. for 3 h. The reaction mixture was concentrated under reduced pressure. The residue was purified by Prep-HPLC on a C18 column eluting with MeCN/H₂O (30%-50%, with 0.1% NH₄HCO₃) to afford the title compound (10.4 mg, 29% yield) as a white solid. ¹H NMR (400 MHz, DMSO-d₆) δ ppm 12.22 (s, 1H), 10.36 (s, 1H), 9.36 (s, 1H), 8.68 (s, 1H), 8.06 (d, J=8.6 Hz, 1H), 7.97 (s, 1H), 7.72 (d, J=8.3 Hz, 1H), 6.84 (s, 1H), 5.33 (s, 2H), 4.39 (d, J=13.2 Hz, 1H), 4.28 (dd, J=32.9, 7.8 Hz, 4H), 3.91 (d, J=12.1 Hz, 1H), 3.81 (t, J=5.4 Hz, 2H), 3.45 (d, J=41.0 Hz, 2H), 3.26 (s, 2H), 3.00 (d, J=7.3 Hz, 2H), 2.78 (dd, J=33.2, 11.5 Hz, 4H), 2.47 (s, 3H), 1.19 (d, J=7.6 Hz, 3H). LCMS calc. for C₃₃H₃₅ClF₃N₁₀O₀₆ [M+H]⁺: m/z=759.2; Found: 759.2.

The following compounds listed in Table 10 were prepared by using an appropriate acid and Example 38 or INT B13 as the methods analogous to those described for Method I. DIEA can be replaced by TEA in the condensation condition of method I.

TABLE 10
Preparations of Examples (Ex)

| | | | | LCMS |
|---|---|---|---|---|
| | | | | Cacl./ |
| Ex | Acid | Structure | Name | Found |
| 211 | 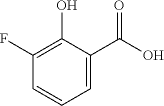 [SMILES: O=C(O)c1cccc(F)c1O] | 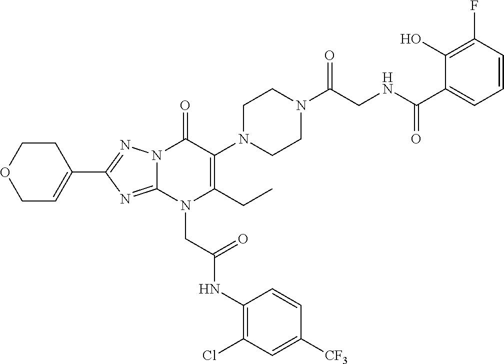 [SMILES: CCc1c(N2CCN(C(=O)CNC(=O)c3cccc(F)c3O)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-3-fluoro-2- hydroxybenzamide | [M + H]⁺: 761.2/ 761.1. |
| 212 | 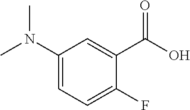 | 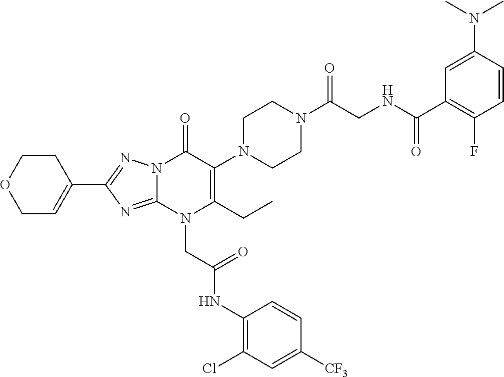 [SMILES: CCc1c(N2CCN(C(=O)CNC(=O)c3cc(N(C)C)ccc3F)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-5-(dimethylamino)-2- fluorobenzamide | [M + H]⁺: 788.3/ 788.2 |
| 213 | 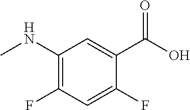 | 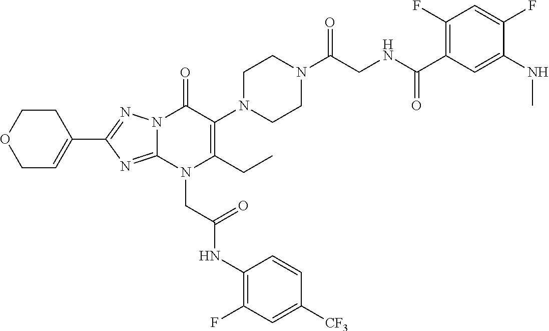 [SMILES: CCc1c(N2CCN(C(=O)CNC(=O)c3cc(NC)c(F)cc3F)CC2)c(=O)n2nc(C3=CCOCC3)nc2n1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-2,4- difluoro-5- (methylamino)benzamide | [M + H]⁺: 776.3/ 776.2 |
| 214 | 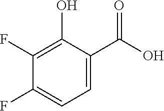 [SMILES: O=C(O)c1ccc(F)c(F)c1O] | 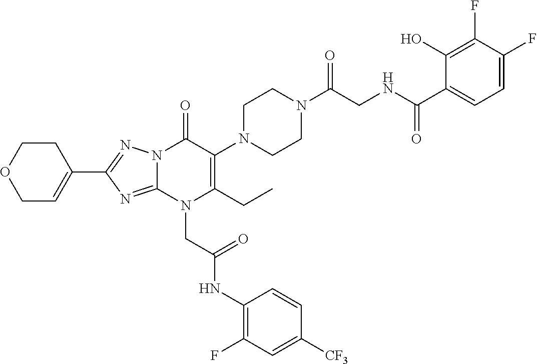 [SMILES: CCc1c(N2CCN(C(=O)CNC(=O)c3ccc(F)c(F)c3O)CC2)c(=O)n2nc(C3=CCOCC3)nc2n1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-3,4- difluoro-2-hydroxybenzamide | [M + H]⁺: 763.2/ 763.1 |
| 215 | 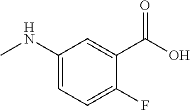 | 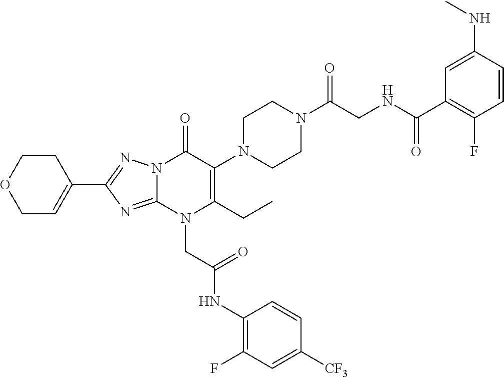 [SMILES: CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1F] | N-(2-(4-(2-(3,6-dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-2- fluoro-5-(methylamino)benzamide | [M − H]⁻: 756.3/ 756.2 |
| 216 | 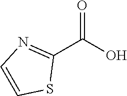 | 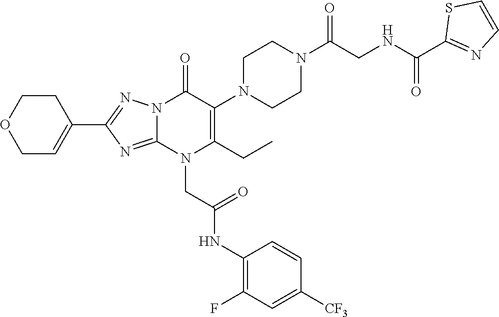 [SMILES: CCc1c(N2CCN(C(=O)CNC(=O)c3nccs3)CC2)c(=O)n2nc(C3=CCOCC3)nc2n1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2- oxoethyl)thiazole-2-carboxamide | [M − H]⁻: 716.2/ 716.1 |
| 217 | 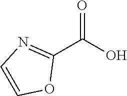 | 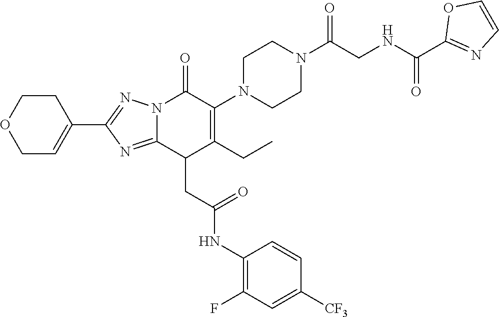 [SMILES: CCC1=C(N2CCN(C(=O)CNC(=O)c3ncco3)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2- oxoethyl)oxazole-2-carboxamide | [M − H]⁻: 700.2/ 700.2 |
| 218 | 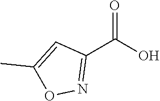 | 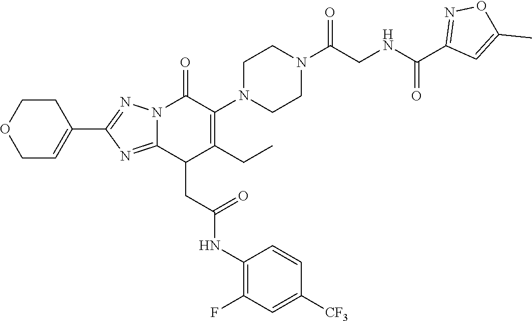 [SMILES: CCC1=C(N2CCN(C(=O)CNC(=O)c3cc(C)on3)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-5- methylisoxazole-3-carboxamide | [M + H]⁺: 716.3/ 716.1 |
| 219 | 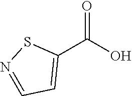 | 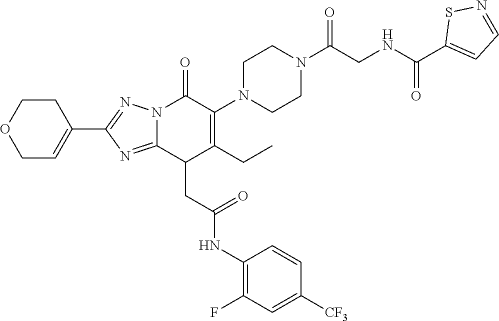 [SMILES: CCC1=C(N2CCN(C(=O)CNC(=O)c3ccns3)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2- oxoethyl)isothiazole-5-carboxamide | [M + H]⁺: 718.2/ 718.1 |
| 220 | 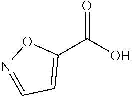 | 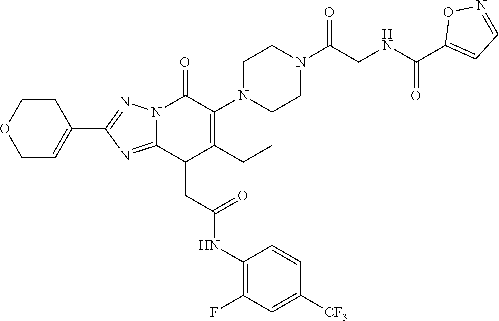 [SMILES: CCC1=C(N2CCN(C(=O)CNC(=O)c3ccno3)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2- oxoethyl)isoxazole-5-carboxamide | [M − H]⁻: 700.2/ 700.2 |
| 221 | 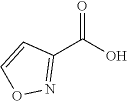 | 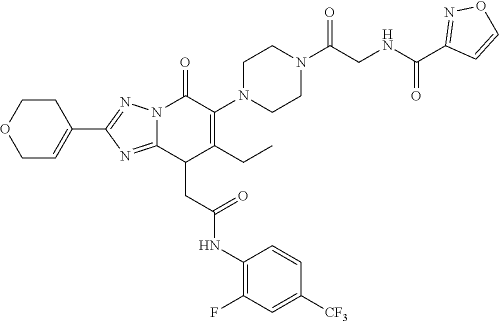 [SMILES: CCC1=C(N2CCN(C(=O)CNC(=O)c3ccon3)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2- oxoethyl)isoxazole-3-carboxamide | [M − H]⁻: 700.2/ 700.2 |
| 222 | 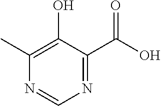 | 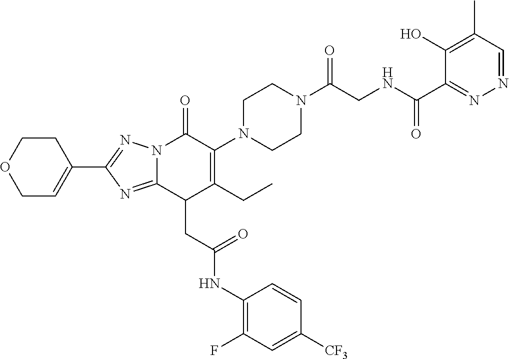 [SMILES: CCC1=C(N2CCN(C(=O)CNC(=O)c3nncc(C)c3O)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-5- hydroxy-6-methylpyrimidine-4- carboxamide | [M + H]⁺: 743.3/ 743.2 |
| 223 | INT C58 | 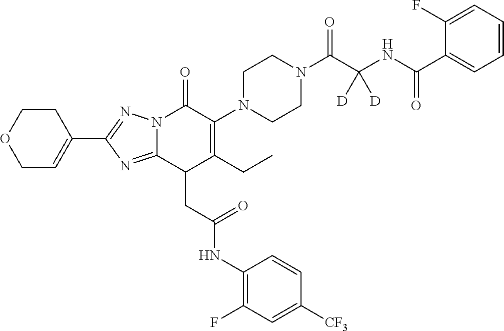 [SMILES: [2H]C([2H])(NC(=O)c1ccccc1F)C(=O)N1CCN(C2=C(CC)C(CC(=O)Nc3ccc(C(F)(F)F)cc3F)c3nc(C4=CCOCC4)nn3C2=O)CC1] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl-1,1- d₂)-2-fluorobenzamide | [M − H]⁻: 729.3/ 729.1 |
| 224 | 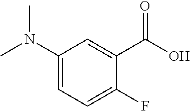 | 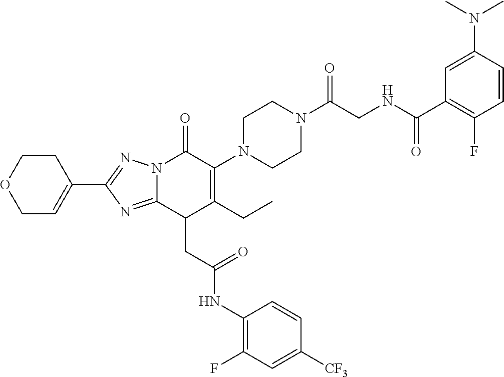 [SMILES: CCC1=C(N2CCN(C(=O)CNC(=O)c3cc(N(C)C)ccc3F)CC2)C(=O)n2nc(C3=CCOCC3)nc2C1CC(=O)Nc1ccc(C(F)(F)F)cc1F] | N-(2-(4-(2-(3,6-Dihydro-2H-pyran- 4-yl)-5-ethyl-4-(2-((2-fluoro-4- (trifluoromethyl)phenyl)amino)-2- oxoethyl)-7-oxo-4,7-dihydro- [1,2,4]triazolo[1,5-a]pyrimidin-6- yl)piperazin-1-yl)-2-oxoethyl)-5- (dimethylamino)-2- fluorobenzamide | [M + H]⁺: 772.3/ 772.2 |

### Example 225: N-(2-(4-(2-(3,6-Dihydro-2H-pyran-4-yl)-5-ethyl-4-(2-((2-fluoro-4-(trifluoromethyl)phenyl)amino)-2-oxoethyl-1,1-d₂)-7-oxo-4,7-dihydro-[1,2,4]triazolo[1,5-a]pyrimidin-6-yl)piperazin-1-yl)-2-oxoethyl-1,1-d₂)-2-fluorobenzamide

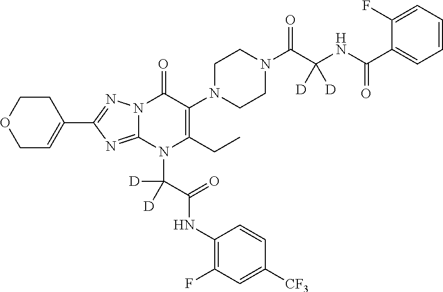 [SMILES: [2H]C([2H])(NC(=O)c1ccccc1F)C(=O)N1CCN(c2c(CC)n(C([2H])([2H])C(=O)Nc3ccc(C(F)(F)F)cc3F)c3nc(C4=CCOCC4)nn3c2=O)CC1]

A mixture of N-(2-(4-(2-(3,6-dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxoethyl-1,1-d₂)-2-fluorobenzamide (30 mg, 0.08 mmol, Example 223), Cs₂CO₃ (98 mg, 0.3 mmol) in DMSO-d₆ (0.4 mL) and D₂O (1 mL) was stirred at 80° C. overnight. The mixture was diluted with water (10 mL) and cooled to r.t. The precipitate was collected by filtration and dried under reduced pressure to afford the title compound (18.7 mg) as a white solid. LCMS calc. for C₃₄H₂₈D₄F₅N₈O₅ [M−H]⁻: m/z=731.3; Found: 731.2.

### Example 226:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(oxazol-2-yl)-2-oxoacetamide

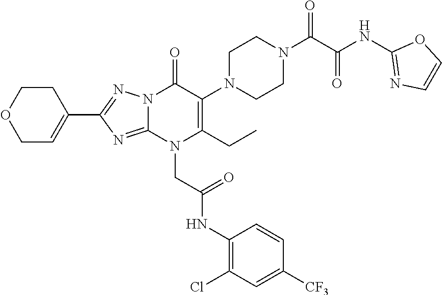 [SMILES: CCc1c(N2CCN(C(=O)C(=O)Nc3ncco3)CC2)c(=O)n2nc(C3=CCOCC3)nc2n1CC(=O)Nc1ccc(C(F)(F)F)cc1Cl]

A mixture of INT B1 (50 mg, 0.09 mmol), INT C59 (21 mg, 0.13 mmol), EDCI (25 mg, 0.13 mmol) in pyridine (1 mL) was stirred at r.t. for 2 h. The reaction mixture was concentrated under reduced pressure. The residue was purified by Prep-HPLC on a C18 column eluting with MeCN/H₂O (20-50%) to afford the title compound (14.8 mg, 24% yield) as a white solid. LCMS calc. for C₃₀H₂₈ClF₃N₉₀₆ [M−H]⁻: m/z=702.2; Found: 702.1.

### Example 227:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(thiazol-2-yl)acetamide

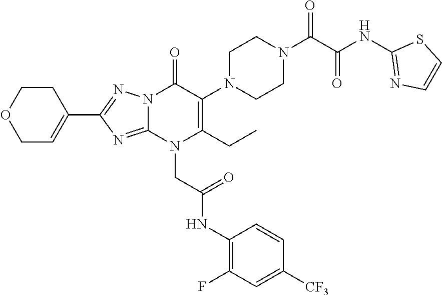 [SMILES: CCc1c(N2CCN(C(=O)C(=O)Nc3nccs3)CC2)c(=O)n2nc(C3=CCOCC3)nc2n1CC(=O)Nc1ccc(C(F)(F)F)cc1F]

To a mixture of INT B13 (50 mg, 0.09 mmol), 2-oxo-2-(thiazol-2-ylamino) acetic acid (21 mg, 0.14 mmol), NMI (22 mg, 0.27 mmol), TCFH (38 mg, 0.14 mmol) in DMF (1 mL) was stirred at r.t. for 2 h. The reaction mixture was concentrated under reduced pressure. The residue was purified by Prep-HPLC on a C18 column eluting with MeCN/H₂O (20-50%) to afford the title compound (19.4 mg, 30% yield) as a white solid. LCMS calc. for: C₃₀H₃₀F₄N₉O₅S [M+H]⁺: m/z=704.2; Found: 704.1.

### Example 228:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(oxazol-2-yl)-2-oxoacetamide

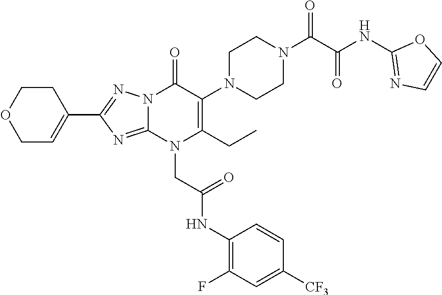 [SMILES: CCc1c(N2CCN(C(=O)C(=O)Nc3ncco3)CC2)c(=O)n2nc(C3=CCOCC3)nc2n1CC(=O)Nc1ccc(C(F)(F)F)cc1F]

A mixture of INT B13 (50 mg, 0.09 mmol), INT C59 (21 mg, 0.14 mmol), EDCI (26 mg, 0.14 mmol) in pyridine (1 mL) was stirred at r.t. for 2 h. The reaction mixture was concentrated under reduced pressure. The residue was purified by Prep-HPLC on a C18 column eluting with MeCN/H₂O (50%) to afford the title compound (14.4 mg, 23% yield) as a white solid. LCMS calc. for C₃₀H₃₀F₄N₉O₆ [M+H]⁺: m/z=688.2; Found: 688.1.

### Example 229:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(pyridin-2-yl)acetamide

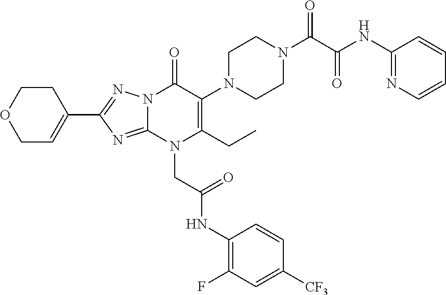 [SMILES: CCc1c(N2CCN(C(=O)C(=O)Nc3ccccn3)CC2)c(=O)n2nc(C3=CCOCC3)nc2n1CC(=O)Nc1ccc(C(F)(F)F)cc1F]

A mixture of INT B13 (30 mg, 0.05 mmol), 2-oxo-2-(pyridin-2-ylamino) acetic acid (14 mg, 0.08 mmol), NMI (13 mg, 0.16 mmol), TCFH (23 mg, 0.08 mmol) in DMF (1 mL) was stirred at r.t. for 2 h. The reaction mixture was concentrated under reduced pressure. The residue was purified by Prep-HPLC on a C18 column eluting with MeCN/H₂O (50%) to afford the title compound (16.3 mg, 31% yield) as a white solid. LCMS calc. for C₃₂H₃₂F₄N₉O₅ [M+H]⁺: m/z=698.2; Found: 698.1. Example 230: N-(2-(4-(2-Bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)benzamide

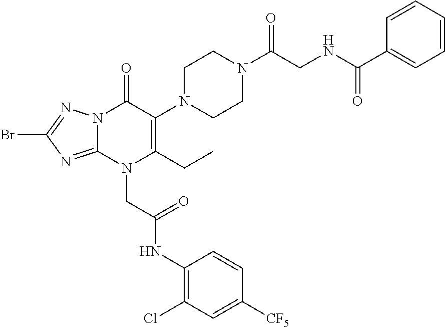

This compound was prepared using procedures analogous to those described for method A using INT B12 and benzoylglycine to afford the title product as a yellow solid. ¹H NMR (400 MHZ, DMSO-d₆) δ ppm 10.34 (s, 1H), 8.60 (t, J=5.7 Hz, 1H), 8.09 (d, J=8.5 Hz, 1H), 7.98 (d, J=1.6 Hz, 1H), 7.89 (dd, J=5.3, 3.3 Hz, 2H), 7.73 (dd, J=8.8, 1.8 Hz, 1H), 7.55 (ddd, J=6.3, 3.6, 1.3 Hz, 1H), 7.52-7.46 (m, 2H), 5.32 (s, 2H), 4.39 (d, J=12.1 Hz, 1H), 4.20 (d, J=5.7 Hz, 2H), 3.97 (d, J=12.3 Hz, 1H), 3.49 (t, J=10.7 Hz, 1H), 3.36 (d, J=12.7 Hz, 1H), 3.25 (d, J=11.6 Hz, 1H), 3.00 (q, J=7.1 Hz, 2H), 2.80 (t, J=12.4 Hz, 1H), 2.73 (d, J=11.1 Hz, 2H), 1.19 (t, J=7.5 Hz, 3H). LCMS calc. for C₂₉H₂₈BrClF₃N₈O₄ [M+H]⁺: m/z=723.1; Found: 723.0.

### Example 231: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)benzamide

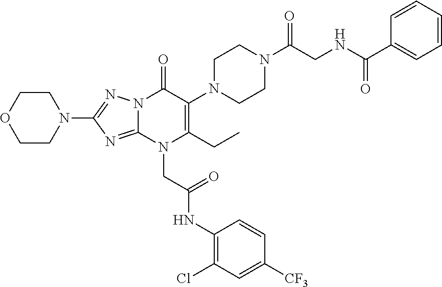 [SMILES: CCc1c(N2CCN(C(=O)CNC(=O)c3ccccc3)CC2)c(=O)n2nc(N3CCOCC3)nc2n1CC(=O)Nc1ccc(C(F)(F)F)cc1Cl]

This compound was prepared by procedures analogous to those described for Method H using benzoylglycine and INT B4 to afford the title product as a white solid. ¹H NMR (400 MHz, DMSO-d₆) δ ppm 10.34 (s, 1H), 8.59 (t, J=5.7 Hz, 1H), 8.05 (d, J=8.5 Hz, 1H), 7.96 (s, 1H), 7.91-7.87 (m, 2H), 7.73 (d, J=8.6 Hz, 1H), 7.55 (t, J=7.2 Hz, 1H), 7.49 (t, J=7.3 Hz, 2H), 5.22 (s, 2H), 4.38 (d, J=12.4 Hz, 1H), 4.19 (d, J=5.7 Hz, 2H), 3.96 (d, J=12.2 Hz, 1H), 3.68-3.64 (m, 4H), 3.51 (t, J=10.9 Hz, 1H), 3.40-3.37 (m, 4H), 3.29-3.19 (m, 2H), 2.95 (q, J=6.9 Hz, 2H), 2.78 (t, J=12.0 Hz, 1H), 2.69 (d, J=11.3 Hz, 2H), 1.17 (t, J=7.4 Hz, 3H). LCMS calc. for C₃₃H₃₆ClF₃N₉O₅ [M+H]⁺: m/z=730.2; Found: 730.0.

### Example 232: N-(2-(4-(4-(2-((2-Chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazin-1-yl)-2-oxoethyl)benzamide

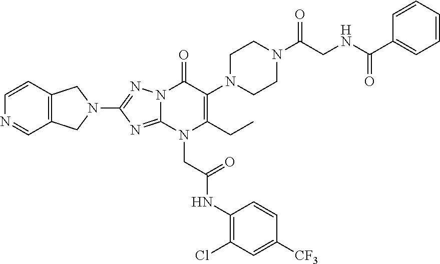 [SMILES: CCc1c(N2CCN(C(=O)CNC(=O)c3ccccc3)CC2)c(=O)n2nc(N3Cc4ccncc4C3)nc2n1CC(=O)Nc1ccc(C(F)(F)F)cc1Cl]

This compound was prepared by procedures analogous to those described for Method H using benzoylglycine and INT B5 to afford the title product as a white solid. ¹H NMR (400 MHZ, DMSO-d₆) δ ppm 10.41 (s, 1H), 8.73 (s, 1H), 8.60 (t, J=5.4 Hz, 2H), 8.08 (d, J=8.6 Hz, 1H), 7.98 (s, 1H), 7.90 (d, J=7.2 Hz, 2H), 7.73 (d, J=8.8 Hz, 1H), 7.64 (d, J=5.2 Hz, 1H), 7.56 (t, J=7.2 Hz, 1H), 7.50 (t, J=7.4 Hz, 2H), 5.28 (s, 2H), 4.83 (s, 4H), 4.39 (d, J=12.0 Hz, 1H), 4.21 (d, J=4.5 Hz, 2H), 3.97 (d, J=11.7 Hz, 1H), 3.53 (s, 1H), 3.25 (d, J=11.9 Hz, 2H), 2.97 (d, J=7.3 Hz, 2H), 2.80 (s, 1H), 2.71 (d, J=10.7 Hz, 2H), 1.19 (dd, J=9.9, 5.0 Hz, 3H). LCMS calc. for C₃₆H₃₅ClF₃N₁₀O₄ [M+H]⁺: m/z=763.2; Found: 763.3.

### Example 233: N-(2-(4-(4-(2-((2-Chloro-4-(trifluoromethyl)phenyl)amino)-2-oxoethyl)-5-ethyl-7-oxo-2-(tetrahydro-1H-furo[3,4-c]pyrrol-5 (3H)-yl)-4,7-dihydro-[1,2,4]triazolo[1,5-a]pyrimidin-6-yl)piperazin-1-yl)-2-oxoethyl)benzamide

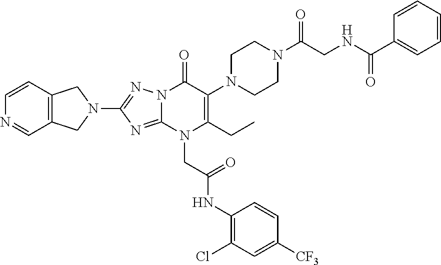 [SMILES: CCc1c(N2CCN(C(=O)CNC(=O)c3ccccc3)CC2)c(=O)n2nc(N3Cc4ccncc4C3)nc2n1CC(=O)Nc1ccc(C(F)(F)F)cc1Cl]

A mixture of N-(2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)benzamide (20 mg, 0.03 mmol, Example 230), TEA (3.0 mg; 0.03 mmol), KOAc (8.8 mg, 0.09 mmol) and hexahydro-1H-furo[3,4-c]pyrrole hydrochloride (8.9 mg, 0.06 mmol) in DMSO (2 mL) was stirred at 110° C. for 16 h. The reaction mixture was concentrated under reduced pressure. The residue was purified by Prep-HPLC on a C18 column eluting with MeCN/H₂O (20-60%, with 0.1% FA) to afford the title compound (4.4 mg, 20% yield) as a white solid. ¹H NMR (400 MHZ, DMSO-d₆) δ ppm 10.34 (s, 1H), 8.59 (s, 1H), 8.06 (d, J=8.7 Hz, 1H), 7.97 (s, 1H), 7.92-7.86 (m, 2H), 7.73 (d, J=7.1 Hz, 1H), 7.54 (d, J=7.2 Hz, 1H), 7.49 (t, J=7.2 Hz, 2H), 5.22 (s, 2H), 4.38 (d, J=11.5 Hz, 1H), 4.19 (d, J=5.7 Hz, 2H), 3.96 (d, J=11.2 Hz, 1H), 3.81 (dd, J=8.6, 6.6 Hz, 2H), 3.51 (dd, J=9.8, 6.6 Hz, 4H), 3.30 (m, 6H), 2.94 (d, J=7.9 Hz, 4H), 2.74 (d, J=33.4 Hz, 2H), 1.17 (t, J=7.5 Hz, 3H). LCMS calc. for C₃₅H₃₈ClF₃N₉O₅ [M+H]⁺: m/z=756.3; Found: 756.5. Example 234: N-(2-(4-(4-(2-((2-Chloro-4-(trifluoromethyl)phenyl)amino)-2-oxoethyl)-2-(2,3-dihydro-1H-pyrrolo[3,2-b]pyridin-1-yl)-5-ethyl-7-oxo-4,7-dihydro-[1,2,4]triazolo[1,5-a]pyrimidin-6-yl)piperazin-1-yl)-2-oxoethyl)benzamide

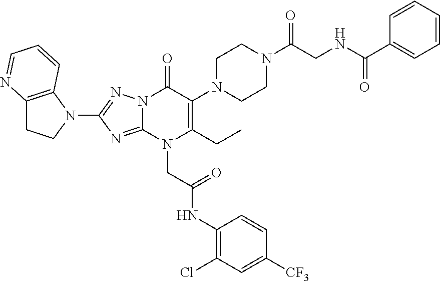 [SMILES: CCc1c(N2CCN(C(=O)CNC(=O)c3ccccc3)CC2)c(=O)n2nc(N3CCc4ncccc43)nc2n1CC(=O)Nc1ccc(C(F)(F)F)cc1Cl]

To a solution of N-(2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)benzamide (50 mg, 0.07 mmol, Example 230) in DMSO (2 mL) was added Brettphos Pd G3 (6.25 mg, 0.007 mmol), K₃PO₄ (43.88 mg, 0.21 mmol) and 2,3-dihydro-1H-pyrrolo[3,2-b]pyridine (9.93 mg, 0.08 mmol). The mixture was stirred at 110° C. for 2 h. The reaction mixture was quenched with H₂O (5 mL) and extracted with DCM (5 mL×3). The combined organic layers were washed with sat. NH₄Cl aq. (10 mL) and brine (10 mL), dried over anhydrous Na₂SO₄, filtered and concentrated. The residue was purified by Prep-HPLC on a C18 column eluting with MeCN/H₂O (5-95%, with 0.1% NH₄HCO₃) to afford the title compound (19.5 mg, 37% yield) as a white solid. ¹H NMR (400 MHZ, DMSO-d₆) δ ppm 10.46 (s, 1H), 8.60 (t, J=5.7 Hz, 1H), 8.11 (dd, J=8.1, 1.3 Hz, 1H), 8.04 (d, J=8.7 Hz, 1H), 8.00-7.94 (m, 2H), 7.93-7.87 (m, 2H), 7.72 (d, J=9.0 Hz, 1H), 7.54 (dd, J=6.7, 1.9 Hz, 1H), 7.49 (dd, J=11.4, 4.5 Hz, 2H), 7.08 (dd, J=7.5, 5.5 Hz, 1H), 5.35 (s, 2H), 4.40 (d, J=11.4 Hz, 1H), 4.21 (d, J=5.3 Hz, 2H), 4.11 (t, J=8.7 Hz, 2H), 3.98 (d, J=11.8 Hz, 1H), 3.52 (d, J=10.4 Hz, 1H), 3.41 (s, 2H), 3.26 (s, 2H), 3.03 (d, J=7.5 Hz, 2H), 2.83 (d, J=12.8 Hz, 1H), 2.74 (d, J=10.6 Hz, 2H), 1.21 (t, J=7.5 Hz, 3H). LCMS calc. for C₃₆H₃₅ClF₃N₁₀O₄ [M+H]⁺: m/z=763.2; Found: 763.2.

### Example 235: N-(2-(4-(4-(2-((2-Chloro-4-(trifluoromethyl)phenyl)amino)-2-oxoethyl)-5-ethyl-7-oxo-2-(2-oxa-6-azaspiro[3.3]heptan-6-yl)-4,7-dihydro-[1,2,4]triazolo[1,5-a]pyrimidin-6-yl)piperazin-1-yl)-2-oxoethyl)benzamide

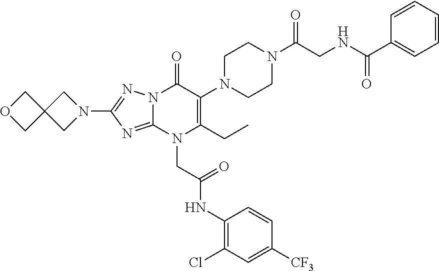 [SMILES: CCc1c(N2CCN(C(=O)CNC(=O)c3ccccc3)CC2)c(=O)n2nc(N3CC4(COC4)C3)nc2n1CC(=O)Nc1ccc(C(F)(F)F)cc1Cl]

This compound was prepared using procedures analogous to those described for Example 233 using 2-oxa-6-azaspiro[3.3]heptane to replace and hexahydro-1H-furo[3,4-c]pyrrole hydrochloride to afford the title product as a white solid. ¹H NMR (400 MHZ, DMSO-d₆)₈ ppm 10.32 (s, 1H), 8.59 (t, J=5.7 Hz, 1H), 8.07 (d, J=8.6 Hz, 1H), 7.97 (d, J=1.6 Hz, 1H), 7.91-7.87 (m, 2H), 7.73 (dd, J=8.7, 1.6 Hz, 1H), 7.57-7.53 (m, 1H), 7.51-7.46 (m, 2H), 5.23 (s, 2H), 4.69 (s, 4H), 4.37 (d, J=11.9 Hz, 1H), 4.19 (d, J=5.7 Hz, 2H), 4.13 (s, 4H), 3.95 (d, J=12.1 Hz, 1H), 3.50 (t, J=10.8 Hz, 1H), 3.39 (d, J=10.0 Hz, 1H), 3.24 (t, J=11.3 Hz, 1H), 2.93 (q, J=7.1 Hz, 2H), 2.78 (t, J=11.2 Hz, 1H), 2.68 (dd, J=6.0, 4.1 Hz, 2H), 1.16 (t, J=7.5 Hz, 3H). LCMS calc. for C₃₄H₃₆ClF₃N₉O₅ [M+H]⁺: m/z=742.2; Found: 742.2.

### Example 236: N-(2-(4-(4-(2-((2-Chloro-4-(trifluoromethyl)phenyl)amino)-2-oxoethyl)-2-(3,4-dihydro-2H-pyrano[2,3-b]pyridin-6-yl)-5-ethyl-7-oxo-4,7-dihydro-[1,2,4]triazolo[1,5-a]pyrimidin-6-yl)piperazin-1-yl)-2-oxoethyl)benzamide

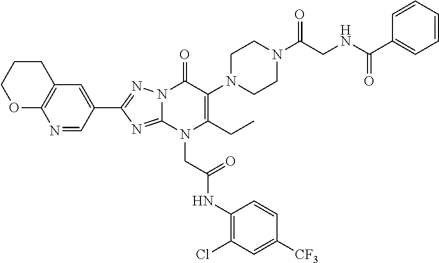 [SMILES: CCc1c(N2CCN(C(=O)CNC(=O)c3ccccc3)CC2)c(=O)n2nc(-c3cnc4c(c3)CCCO4)nc2n1CC(=O)Nc1ccc(C(F)(F)F)cc1Cl]

This compound was prepared using procedures analogous to those described for method A using INT B14 and benzoylglycine to afford the title product as a white solid. ¹H NMR (400 MHZ, DMSO-d₆) δ ppm 10.59 (s, 1H), 8.68 (d, J=2.3 Hz, 1H), 8.62 (d, J=5.7 Hz, 1H), 8.16 (s, 1H), 8.05 (d, J=8.4 Hz, 1H), 7.90 (d, J=7.0 Hz, 3H), 7.68 (s, 1H), 7.55 (d, J=7.2 Hz, 1H), 7.49 (t, J=7.3 Hz, 2H), 5.37 (s, 2H), 4.40 (d, J=10.8 Hz, 1H), 4.34 (d, J=4.9 Hz, 2H), 4.20 (s, 2H), 3.99 (d, J=12.2 Hz, 1H), 3.51 (s, 1H), 3.41 (s, 2H), 3.03 (d, J=6.6 Hz, 3H), 2.87 (d, J=6.2 Hz, 2H), 2.75 (d, J=12.4 Hz, 2H), 1.95 (s, 2H), 1.20 (s, 3H). LCMS calc. for C₃₇H₃₆ClF₃N₉O₅S [M+H]⁺: m/z=778.2; Found: 778.5.

### Example 237: N-(2-(4-(2-(Tert-butyl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)benzamide

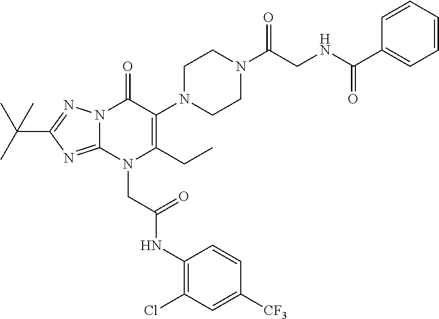 [SMILES: CCc1c(N2CCN(C(=O)CNC(=O)c3ccccc3)CC2)c(=O)n2nc(C(C)(C)C)nc2n1CC(=O)Nc1ccc(C(F)(F)F)cc1Cl]

This compound was prepared using procedures analogous to those described for method A using INT B15 and benzoylglycine to afford the title product as a white solid. ¹H NMR (400 MHZ, DMSO-d₆) δ ppm 10.34 (s, 1H), 8.60 (t, J=5.6 Hz, 1H), 8.00 (d, J=8.4 Hz, 1H), 7.97 (d, J=1.6 Hz, 1H), 7.93-7.83 (m, 2H), 7.73 (dd, J=8.8, 1.6 Hz, 1H), 7.59-7.44 (m, 3H), 5.30 (s, 2H), 4.39 (d, J=12.0 Hz, 1H), 4.20 (d, J=5.6 Hz, 2H), 3.97 (d, J=12.4 Hz, 1H), 3.52 (t, J=10.8 Hz, 1H), 3.43-3.36 (m, 1H), 3.30-3.20 (m, 1H), 2.99 (q, J=7.2 Hz, 2H), 2.80 (t, J=12.0 Hz, 1H), 2.71 (d, J=10.8 Hz, 2H), 1.32 (s, 9H), 1.19 (t, J=7.6 Hz, 3H). LCMS calc. for C₃₃H₃₇ClF₃N₈O₄ [M+H]⁺: m/z=701.3; Found: 701.2.

### Example 238: N-(2-(4-(4-(2-((2-Chloro-4-(trifluoromethyl)phenyl)amino)-2-oxoethyl)-2-cyclopropyl-5-ethyl-7-oxo-4,7-dihydro-[1,2,4]triazolo[1,5-a]pyrimidin-6-yl)piperazin-1-yl)-2-oxoethyl)benzamide

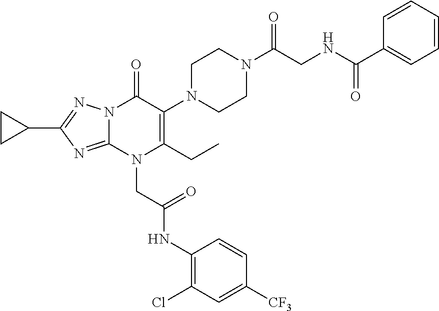 [SMILES: CCc1c(N2CCN(C(=O)CNC(=O)c3ccccc3)CC2)c(=O)n2nc(C3CC3)nc2n1CC(=O)Nc1ccc(C(F)(F)F)cc1Cl]

This compound was prepared using procedures analogous to those described for method A using INT B16 and benzoylglycine to afford the title product as a white solid. ¹H NMR (400 MHZ, DMSO-d₆) δ ppm 10.30 (s, 1H), 8.59 (t, J=5.6 Hz, 1H), 8.04 (d, J=8.4 Hz, 1H), 7.97 (d, J=1.6 Hz, 1H), 7.93-7.86 (m, 2H), 7.73 (dd, J=8.8, 1.6 Hz, 1H), 7.58-7.46 (m, 3H), 5.26 (s, 2H), 4.38 (d, J=12.4 Hz, 1H), 4.20 (d, J=5.6 Hz, 2H), 3.96 (d, J=12.4 Hz, 1H), 3.51 (t, J=10.4 Hz, 1H), 3.43-3.35 (m, 1H), 3.25 (t, J=11.6 Hz, 1H), 3.03-2.92 (m, 2H), 2.79 (t, J=12.4 Hz, 1H), 2.71 (d, J=11.2 Hz, 2H), 2.10-2.00 (m, 1H), 1.18 (t, J=7.2 Hz, 3H), 1.03-0.97 (m, 2H), 0.92-0.85 (m, 2H). LCMS calc. for C₃₂H₃₃ClF₃N₈O₄ [M+H]⁺: m/z=685.2; Found: 685.2.

### Example 239: N-(2-(4-(4-(2-((2-Chloro-4-(trifluoromethyl)phenyl)amino)-2-oxoethyl)-2-ethoxy-5-ethyl-7-oxo-4,7-dihydro-[1,2,4]triazolo[1,5-a]pyrimidin-6-yl)piperazin-1-yl)-2-oxoethyl)-2-fluorobenzamide

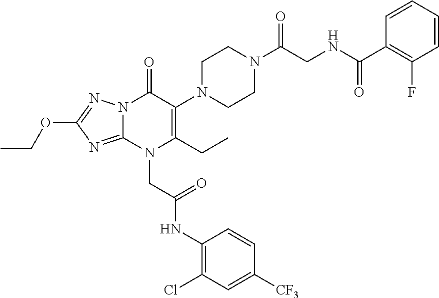 [SMILES: CCOc1nc2n(CC(=O)Nc3ccc(C(F)(F)F)cc3Cl)c(CC)c(N3CCN(C(=O)CNC(=O)c4ccccc4F)CC3)c(=O)n2n1]

A mixture of (2-fluorobenzoyl)glycine (29.72 mg, 0.15 mmol), HOBt (12.29 mg, 0.09 mmol), EDCI (17.25 mg, 0.09 mmol), NMP (8.92 mg, 0.09 mmol) and DIPEA (29.03 mg, 0.23 mmol) in DCM (3 mL) was stirred at r.t. for 30 min. To the mixture INT B17 (40 mg, 0.075 mmol) was added. The mixture was stirred at r.t. for 2 h., and concentrated under reduced pressure. The residue was purified by Prep-HPLC on a C18 column eluting with MeOH/H₂O (45-75% with 0.1% NH₄HCO₃) to afford the title compound (10.5 mg, 19.6% yield) as a white solid. ¹H NMR (400 MHZ, DMSO-d₆) δ ppm 10.16 (s, 1H), 8.33 (q, J=5.2 Hz, 1H), 8.09 (d, J=8.4 Hz, 1H), 7.97 (d, J=1.4 Hz, 1H), 7.79-7.71 (m, 2H), 7.61-7.54 (m, 1H), 7.36-7.30 (m, 2H), 5.24 (s, 2H), 4.39 (d, J=12.2 Hz, 1H), 4.33 (q, J=7.0 Hz, 2H), 4.28-4.16 (m, 2H), 3.91 (d, J=12.0 Hz, 1H), 3.50 (t, J=10.6 Hz, 1H), 3.39 (d, J=11.8 Hz, 1H), 3.24 (t, J=11.6 Hz, 1H), 2.97 (d, J=7.4 Hz, 2H), 2.81 (t, J=11.8 Hz, 1H), 2.71 (d, J=11.0 Hz, 2H), 1.34 (t, J=7.0 Hz, 3H), 1.18 (t, J=7.6 Hz, 3H). LCMS calc. for C₃₁H₃₂ClF₄N₈O₅ [M+H]⁺: m/z=707.2; Found: 707.2.

Example 240: N-(2-(4-(4-(2-((2-Chloro-4-(trifluoromethyl)phenyl)amino)-2-oxoethyl)-5-ethyl-2-(2-methoxyethoxy)-7-oxo-4,7-dihydro-[1,2,4]triazolo[1,5-a]pyrimidin-6-yl)piperazin-1-yl)-2-oxoethyl)-2-fluorobenzamide

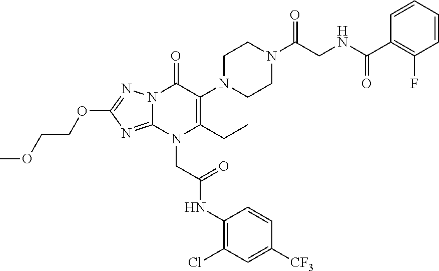 [SMILES: CCc1c(N2CCN(C(=O)CNC(=O)c3ccccc3F)CC2)c(=O)n2nc(OCCOC)nc2n1CC(=O)Nc1ccc(C(F)(F)F)cc1Cl]

This compound was prepared using procedures analogous to those described for method B using INT B18 and (2-fluorobenzoyl)glycine to afford the title product as a white solid. ¹H NMR (400 MHZ, DMSO-d₆) δ ppm 10.34 (s, 1H), 8.34-8.32 (m, 1H), 8.09 (d, J=8.4 Hz, 1H), 7.97 (d, J=1.6 Hz, 1H), 7.80-7.70 (m, 2H), 7.61-7.54 (m, 1H), 7.35-7.30 (m, 2H), 5.24 (s, 2H), 4.43-4.34 (m, 3H), 4.29-4.15 (m, 2H), 3.91 (d, J=12.4 Hz, 1H), 3.69-3.61 (m, 2H), 3.55-3.35 (m, 2H), 3.28 (s, 3H), 3.24-3.21 (m, 1H), 2.98-2.96 (m, 2H), 2.81 (t, J=11.6 Hz, 1H), 2.71 (d, J=11.2 Hz, 2H), 1.18 (t, J=7.6 Hz, 3H). LCMS calc. for C₃₂H₃₄ClF₄N₈O₆ [M+H]⁺: m/z=737.2; Found: 737.8.

### Example 241: N-(2-(4-(4-(2-((2-Chloro-4-(trifluoromethyl)phenyl)amino)-2-oxoethyl)-5-ethyl-2-(methylthio)-7-oxo-4,7-dihydro-[1,2,4]triazolo[1,5-a]pyrimidin-6-yl)piperazin-1-yl)-2-oxoethyl)-2-fluorobenzamide

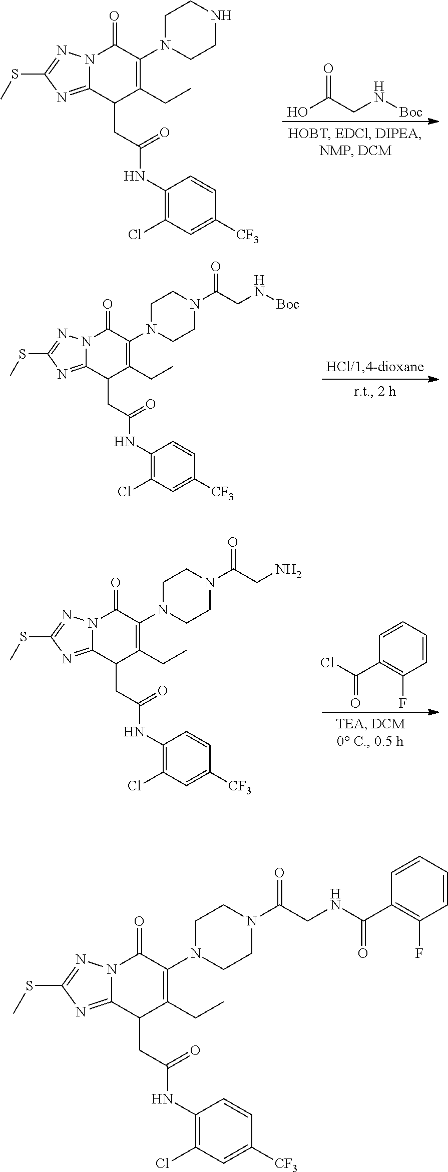 [SMILES: CCC1=C(N2CCNCC2)C(=O)n2nc(SC)nc2C1CC(=O)Nc1ccc(C(F)(F)F)cc1Cl ; CC(C)(C)OC(=O)NCC(=O)O ; CCC1=C(N2CCN(C(=O)CNC(=O)OC(C)(C)C)CC2)C(=O)n2nc(SC)nc2C1CC(=O)Nc1ccc(C(F)(F)F)cc1Cl ; CCC1=C(N2CCN(C(=O)CN)CC2)C(=O)n2nc(SC)nc2C1CC(=O)Nc1ccc(C(F)(F)F)cc1Cl ; O=C(Cl)c1ccccc1F ; CCC1=C(N2CCN(C(=O)CNC(=O)c3ccccc3F)CC2)C(=O)n2nc(SC)nc2C1CC(=O)Nc1ccc(C(F)(F)F)cc1Cl]

### Step 1: tert-butyl (2-(4-(4-(2-((2-chloro-4-(trifluoromethyl)phenyl)amino)-2-oxoethyl)-5-ethyl-2-(methylthio)-7-oxo-4,7-dihydro-[1,2,4]triazolo[1,5-a]pyrimidin-6-yl)piperazin-1-yl)-2-oxoethyl)carbamate

A mixture of INT B 19 (200 mg, 0.38 mmol), (tert-butoxycarbonyl)glycine (132 mg, 0.76 mmol), HOBt (61 mg, 0.45 mmol), NMP (45 mg, 0.45 mmol), EDCI (87 mg, 0.43 mmol) and DIPEA (146 mg, 1.13 mmol) in DCM (3 mL) was stirred at r.t. for 2 h. The reaction mixture was concentrated under reduced pressure. The residue was purified by Prep-HPLC on a C18 column eluting with MeCN/H₂O (60%, with 0.05% NH₄HCO₃) to afford the title product (140 mg, 54% yield) as a white solid. LCMS calc. for C₂₈H₃₅ClF₃N₈O₅S [M+H]⁺: m/z=687.2; Found: 687.4.

### Step 2: N-(2-chloro-4-(trifluoromethyl)phenyl)-2-(5-ethyl-6-(4-glycylpiperazin-1-yl)-2-(methylthio)-7-oxo-[1,2,4]triazolo[1,5-a]pyrimidin-4(7H)-yl)acetamide

To the solution of tert-butyl (2-(4-(4-(2-((2-chloro-4-(trifluoromethyl)phenyl)amino)-2-oxoethyl)-5-ethyl-2-(methylthio)-7-oxo-4,7-dihydro-[1,2,4]triazolo[1,5-a]pyrimidin-6-yl)piperazin-1-yl)-2-oxoethyl)carbamate (140 mg, 0.20 mmol) in DCM (1 mL) was added 4 M HCl/dioxane (5 mL). The mixture was stirred at r.t. for 1 h., and concentrated under reduced pressure to afford the title product as a HCl salt (120 mg, crude) as a yellow solid. LCMS calc. for C₂₃H₂₇ClF₃N₈O₃S [M+H]⁺: m/z=587.1; Found: 587.3.

### Step 3: N-(2-(4-(4-(2-((2-chloro-4-(trifluoromethyl)phenyl)amino)-2-oxoethyl)-5-ethyl-2-(methylthio)-7-oxo-4,7-dihydro-[1,2,4]triazolo[1,5-a]pyrimidin-6-yl)piperazin-1-yl)-2-oxoethyl)-2-fluorobenzamide

To the solution of N-(2-chloro-4-(trifluoromethyl)phenyl)-2-(5-ethyl-6-(4-glycyl piperazin-1-yl)-2-(methylthio)-7-oxo-[1,2,4]triazolo[1,5-a]pyrimidin-4(7H)-yl)acetamide HCl salt (30 mg, 0.05 mmol) in DCM (0.8 mL) was added TEA (26 mg, 0.26 mmol) at 0° C. and stirred for 10 min. Then 2-fluorobenzoyl chloride (6 mg, 0.04 mmol) in DCM (0.2 mL) was added and stirred at r.t. for 0.5 h. The reaction mixture was extracted with DCM (10 mL×2). The combined organic layers were washed with H₂O (10 mL), dried over anhydrous Na₂SO₄, filtered and concentrated. The residue was purified by Prep-HPLC on a C18 column eluting with MeCN/H₂O (65-75%, with 0.1% NH₄HCO₃) to afford the title product (16.4 mg, 45.4% yield) as a white solid. ¹H NMR (400 MHZ, DMSO-d₆) δ ppm 10.34 (s, 1H), 8.33 (q, J=5.1 Hz, 1H), 8.12-8.06 (m, 1H), 7.98 (d, J=1.7 Hz, 1H), 7.79-7.70 (m, 2H), 7.58 (ddd, J=15.5, 5.3, 1.8 Hz, 1H), 7.36-7.30 (m, 2H), 5.30 (d, J=11.7 Hz, 2H), 4.40 (d, J=12.5 Hz, 1H), 4.30-4.16 (m, 2H), 3.91 (d, J=12.5 Hz, 1H), 3.50 (t, J=10.4 Hz, 1H), 3.40 (d, J=10.1 Hz, 1H), 3.25 (t, J=11.8 Hz, 1H), 3.03-2.93 (m, 2H), 2.82 (t, J=11.7 Hz, 1H), 2.74 (s, 2H), 2.59 (s, 3H), 1.18 (t, J=7.4 Hz, 3H). LCMS calc. for C₃₀H₃₀ClF₄N₈O₄S [M+H]⁺: m/z=709.2; Found: 709.2.

### Example 242: N-(2-(4-(4-(2-((2-Chloro-4-(trifluoromethyl)phenyl)amino)-2-oxoethyl)-5-ethyl-2-(3-methoxyazetidin-1-yl)-7-oxo-4,7-dihydro-[1,2,4]triazolo[1,5-a]pyrimidin-6-yl)piperazin-1-yl)-2-oxoethyl)-2-fluorobenzamide

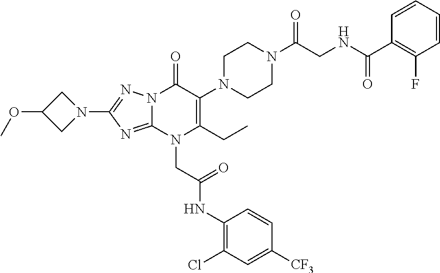 [SMILES: CCc1c(N2CCN(C(=O)CNC(=O)c3ccccc3F)CC2)c(=O)n2nc(N3CC(OC)C3)nc2n1CC(=O)Nc1ccc(C(F)(F)F)cc1Cl]

This compound was prepared using procedures analogous to those described for method I using INT B20 and (2-fluorobenzoyl)glycine to afford the title product as a white solid. LCMS calc. for C₃₃H₃₃ClF₄N₉O₅ [M−H]⁻: m/z=746.2; Found: 746.1.

### Example 243: N-(2-(4-(4-(2-((2-Chloro-4-(trifluoromethyl)phenyl)amino)-2-oxoethyl)-2-(3-cyanoazetidin-1-yl)-5-ethyl-7-oxo-4,7-dihydro-[1,2,4]triazolo[1,5-a]pyrimidin-6-yl)piperazin-1-yl)-2-oxoethyl)-2-fluorobenzamide

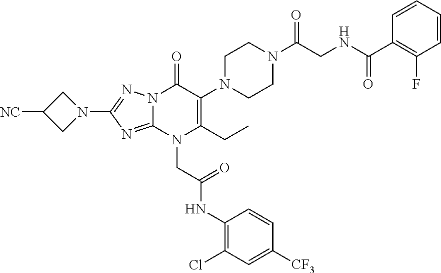 [SMILES: CCc1c(N2CCN(C(=O)CNC(=O)c3ccccc3F)CC2)c(=O)n2nc(N3CC(C#N)C3)nc2n1CC(=O)Nc1ccc(C(F)(F)F)cc1Cl]

This compound was prepared using procedures analogous to those described for method I using INT B21 and (2-fluorobenzoyl)glycine to afford the title product as a white solid. LCMS calc. for C₃₃H₃₀ClF₄N₁₀O₄ [M−H]⁻: m/z=741.2; Found: 741.1.

### Example 244: N-(2-(4-(4-(2-((2-Chloro-4-(trifluoromethyl)phenyl)amino)-2-oxoethyl)-2-(3,3-difluoroazetidin-1-yl)-5-ethyl-7-oxo-4,7-dihydro-[1,2,4]triazolo[1,5-a]pyrimidin-6-yl)piperazin-1-yl)-2-oxoethyl)-2-fluorobenzamide

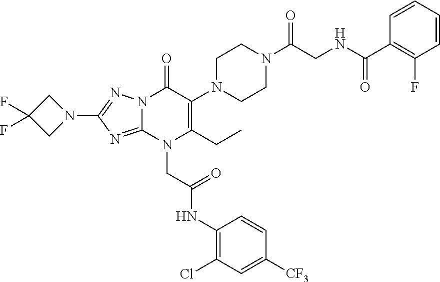 [SMILES: CCc1c(N2CCN(C(=O)CNC(=O)c3ccccc3F)CC2)c(=O)n2nc(N3CC(F)(F)C3)nc2n1CC(=O)Nc1ccc(C(F)(F)F)cc1Cl]

This compound was prepared using procedures analogous to those described for method I using INT B22 and (2-fluorobenzoyl)glycine to afford the title product as a white solid. LCMS calc. for C₃₂H₂₉ClF₆N₉O₄ [M−H]⁻: m/z=752.2; Found: 752.1.

### Example 245: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)-2-fluorobenzamide

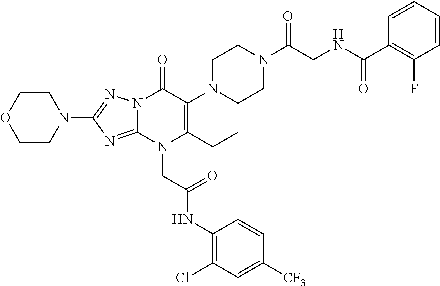 [SMILES: CCc1c(N2CCN(C(=O)CNC(=O)c3ccccc3F)CC2)c(=O)n2nc(N3CCOCC3)nc2n1CC(=O)Nc1ccc(C(F)(F)F)cc1Cl]

To a solution of HOBt (97.9 mg, 0.73 mmol), EDCI (139 mg, 0.73 mmol), NMP (72 mg, 0.73 mmol) and DIPEA (935 mg, 2.42 mmol) in DCM (30 mL) was added (2-fluorobenzoyl)glycine (238.4 mg, 1.21 mmol) at 0° C. The mixture was stirred for 30 min., INT B4 (344 mg, 0.60 mmol) was added and stirred at r.t. for an additional 3 h. The reaction mixture was concentrated under reduced pressure. The residue was purified by Prep-HPLC on a C18 column eluting with MeOH/H₂O (45-75% with 0.1% NH₄HCO₃) to afford the title compound (186.5 mg, 41.2% yield) as a white solid. ¹H NMR (400 MHz, DMSO-d₆) δ ppm 10.35 (s, 1H), 8.32 (q, J=5.2 Hz, 1H), 8.05 (d, J=8.4 Hz, 1H), 7.97 (d, J=1.6 Hz, 1H), 7.78-7.72 (m, 2H), 7.60-7.54 (m, 1H), 7.36-7.30 (m, 2H), 5.22 (s, 2H), 4.39 (d, J=12.0 Hz, 1H), 4.22 (qd, J=16.8, 5.0 Hz, 2H), 3.90 (d, J=12.0 Hz, 1H), 3.68-3.64 (m, 4H), 3.51 (t, J=10.6 Hz, 1H), 3.40-3.37 (m, 4H), 3.23 (t, J=11.1 Hz, 2H), 2.95 (dd, J=14.4, 7.0 Hz, 2H), 2.80 (t, J=11.6 Hz, 1H), 2.72-2.67 (m, 2H), 1.17 (t, J=7.6 Hz, 3H). LCMS calc. for C₃₃H₃₅ClF₄N₉O₅ [M+H]⁺: m/z=748.2; Found: 748.2.

### Example 246: N-(2-(4-(4-(2-((2-Chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazin-1-yl)-2-oxoethyl)-2-fluorobenzamide

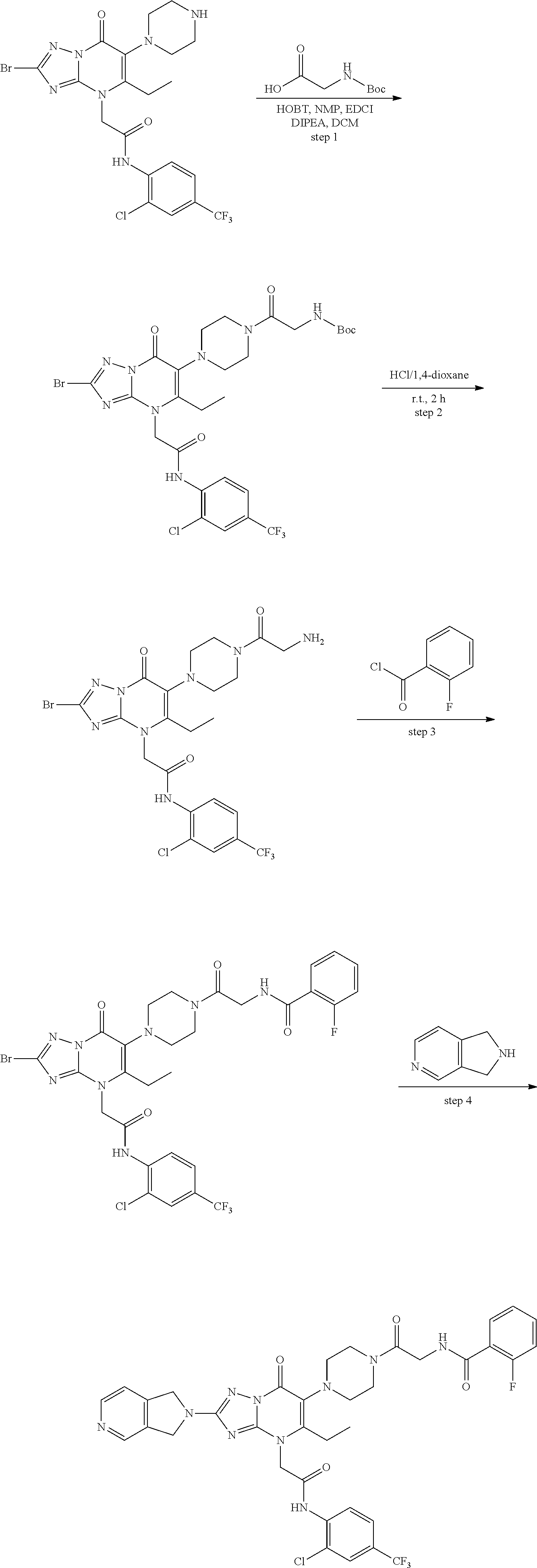

### Step 1: tert-butyl (2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)carbamate

A mixture of 2-(2-bromo-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide (1.8 g, 3.21 mmol, INT B12), (tert-butoxycarbonyl)glycine (1123 mg, 6.42 mmol), HOBt (520 mg, 3.85 mmol), NMP (381 mg, 3.85 mmol), EDCI (739 mg, 3.85 mmol) and DIPEA (1242 mg, 9.63 mmol) in DCM (20 mL) was stirred at r.t. overnight. The reaction mixture was concentrated under reduced pressure. The residue was purified by Prep-HPLC on a C18 column eluting with MeOH/H₂O (60%, with 0.05% NH₄HCO₃) to afford the title product (1.8 g, 78.2% yield) as a white solid. LCMS calc. for C₂₇H₃₂BrClF₃N₈O₅ [M+H]⁺: m/z=719.1; Found: [M+H−100]⁺: 619.1.

### Step 2: 2-(2-bromo-5-ethyl-6-(4-glycylpiperazin-1-yl)-7-oxo-[1,2,4]triazolo[1,5-a]pyrimidin-4(7H)-yl)-N-(2-chloro-4-(trifluoromethyl)phenyl)acetamide

To the solution of tert-butyl (2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)carbamate (1.8 g, 2.51 mmol) in DCM (8 mL) was added 4 M HCl/1,4-dioxane (25 mL). The mixture was stirred at r.t. for 2 h., and concentrated under reduced pressure to afford the title product (1.6 g, crude) as a white solid. LCMS calc. for C₂₂H₂₃BrClF₃N₈O₃ [M+H]⁺: m/z=619.1; Found: 619.0.

### Step 3: N-(2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)-2-fluorobenzamide

To the solution of 2-(2-bromo-5-ethyl-6-(4-glycylpiperazin-1-yl)-7-oxo-[1,2,4]triazolo[1,5-a]pyrimidin-4(7H)-yl)-N-(2-chloro-4-(trifluoromethyl)phenyl)acetamide (1.6 g, 2.59 mmol) and TEA (1.3 g, 12.95 mmol) in DCM (18 mL) was added 2-fluorobenzoyl chloride (409 mg, 2.59 mmol) in DCM (2 mL) dropwise at 0° C. The mixture was stirred at 0° C. for 0.5 h., diluted with water (50 mL), extracted with DCM (100 mL×2), dried over anhydrous Na₂SO₄, filtered and concentrated under reduced pressure. The residue was triturated with EtOAc/PE (3 mL/30 mL) to afford the title product (1.1 g, 57.4% yield) as a white solid. LCMS calc. for C₂₉H₂₇BrClF₄N₈O₄ [M+H]⁺: m/z=741.1; Found: 741.0.

### Step 4: N-(2-(4-(4-(2-((2-chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazin-1-yl)-2-oxoethyl)-2-fluorobenzamide

A mixture of N-(2-(4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazin-1-yl)-2-oxoethyl)-2-fluorobenzamide (50 mg, 0.07 mmol), 2,3-dihydro-1H-pyrrolo[3,4-c]pyridine dihydrochloride (26.02 mg, 0.13 mmol) and KOAc (29.7 mg, 0.40 mmol) in DMSO (5 mL) was stirred at 120° C. for 2 h. The reaction mixture was concentrated under reduced pressure. The residue was purified by Prep-HPLC on a C18 column eluting with MeOH/H₂O (45-95% with 0.1% FA) to give the title compound (2.4 mg, 4.6% yield) as white solid. ¹H NMR (400 MHZ, DMSO-d₆) δ ppm 10.39 (s, 1H), 8.64 (s, 1H), 8.50 (d, J=5.0 Hz, 1H), 8.33 (q, J=5.2 Hz, 1H), 8.08 (d, J=8.4 Hz, 1H), 7.98 (d, J=1.4 Hz, 1H), 7.75 (ddd, J=12.8, 9.2, 1.7 Hz, 2H), 7.61-7.54 (m, 1H), 7.47 (d, J=5.2 Hz, 1H), 7.36-7.30 (m, 2H), 5.28 (s, 2H), 4.78 (d, J=8.4 Hz, 4H), 4.40 (d, J=11.8 Hz, 1H), 4.29-4.16 (m, 2H), 3.91 (d, J=11.2 Hz, 1H), 3.57-3.50 (m, 1H), 3.43 (d, J=11.8 Hz, 1H), 3.25 (t, J=12.2 Hz, 1H), 2.96 (d, J=7.8 Hz, 2H), 2.81 (t, J=11.0 Hz, 1H), 2.71 (d, J=10.4 Hz, 2H), 1.19 (t, J=7.6 Hz, 3H). LCMS calc. for C₃₆H₃₄ClF₄N₁₀O₄ [M+H]⁺: m/z=781.2; Found: 781.7.

### Example 247: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)-2-morpholinobenzamide

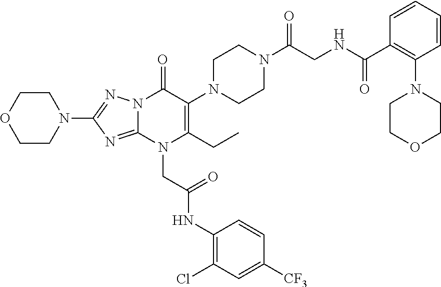 [SMILES: CCc1c(N2CCN(C(=O)CNC(=O)c3ccccc3N3CCOCC3)CC2)c(=O)n2nc(N3CCOCC3)nc2n1CC(=O)Nc1ccc(C(F)(F)F)cc1Cl]

o a solution of N-(2-(4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)-2-fluorobenzamide (125 mg, 0.17 mmol, Example 245) in DMSO (10 mL) was added morpholine (2 mL) and KOAc (98.4 mg, 1.0 mmol). The reaction was stirred at 120° C. for 48 h., and concentrated under reduced pressure. The residue was purified by Prep-HPLC on a C18 column eluting with MeOH/H₂O (45-95% with 0.1% NH₄HCO₃) to get the title compound (8.6 mg, 6.3% yield) as a white solid. ¹H NMR (400 MHZ, DMSO-d₆) δ ppm 10.33 (s, 1H), 10.01 (s, 1H), 8.05 (d, J=8.4 Hz, 1H), 7.97 (s, 1H), 7.92 (d, J=6.4 Hz, 1H), 7.73 (d, J=8.6 Hz, 1H), 7.51 (t, J=7.0 Hz, 1H), 7.34 (d, J=8.0 Hz, 1H), 7.22 (t, J=7.6 Hz, 1H), 5.22 (s, 2H), 4.47-4.34 (m, 2H), 4.24 (dd, J=17.2, 4.4 Hz, 1H), 3.88-3.83 (m, 4H), 3.67-3.65 (m, 4H), 3.54-3.47 (m, 1H), 3.44-3.42 (m, 1H), 3.40-3.38 (m, 4H), 3.30-3.18 (m, 2H), 2.96-2.94 (m, 6H), 2.86-2.79 (m, 1H), 2.72-2.69 (m, 2H), 1.18 (t, J=7.2 Hz, 3H). LCMS calc. for C₃₇H₄₃ClF₃N₁₀O₆ [M+H]⁺: m/z=815.3; Found: 815.5.

### Example 248: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)thiazole-2-carboxamide

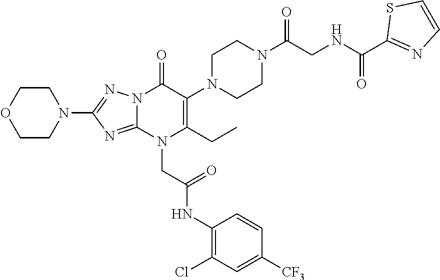 [SMILES: CCc1c(N2CCN(C(=O)CNC(=O)c3nccs3)CC2)c(=O)n2nc(N3CCOCC3)nc2n1CC(=O)Nc1ccc(C(F)(F)F)cc1Cl]

### Step 1: tert-butyl (2-(4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)carbamate

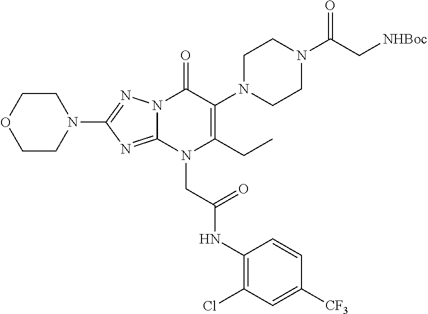 [SMILES: CCc1c(N2CCN(C(=O)CNC(=O)OC(C)(C)C)CC2)c(=O)n2nc(N3CCOCC3)nc2n1CC(=O)Nc1ccc(C(F)(F)F)cc1Cl]

A mixture of (tert-butoxycarbonyl)glycine (260 mg, 1.5 mmol), INT B4 (57 mg, 1 mmol), EDCI (76 mg, 2 mmol), HOBt (52 mg, 2 mmol) and TEA (300 mg, 3 mmol) in DMF (3 mL) was stirred at r.t. overnight. The mixture was concentrated under reduced pressure. The residue was purified by flash chromatography on a C18 column eluting with MeCN/H₂O (70-80% with 0.1% NH₄HCO₃) to afford the title compound (60 mg) as a white solid. LCMS calc. for C₃₁H₃₈ClF₃N₉O₆ [M−H]⁻: m/z=724.3; Found: 724.2.

### Step 2: N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide

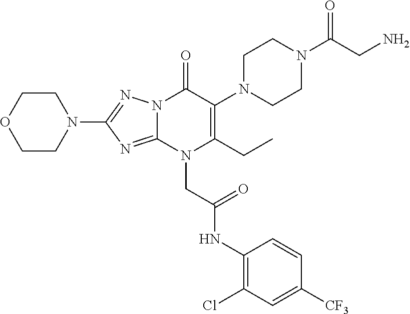 [SMILES: CCc1c(N2CCN(C(=O)CN)CC2)c(=O)n2nc(N3CCOCC3)nc2n1CC(=O)Nc1ccc(C(F)(F)F)cc1Cl]

A mixture of tert-butyl (2-(4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)carbamate (55 mg, 0.08 mmol) in DCM (1 mL) and 4.0 M HCl/dioxane (1 mL) was stirred at r.t. for 2 h. The precipitate was collected by filtration to afford the title compound (40 mg) as a brown solid. LCMS calc. for C₂₆H₃₂ClF₃N₉O₄ [M+H]⁺: m/z=626.2; Found: 626.2.

### Step 3: N-(2-(4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)thiazole-2-carboxamide

This compound was prepared using procedures analogous to those described for method I using N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide and thiazole-2-carboxylic acid to afford the title product as a white solid. LCMS calc. for C₃₀H₃₃ClF₃N₁₀O₅S [M+H]⁺: m/z=737.2; Found: 737.1.

### Example 249: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)oxazole-2-carboxamide

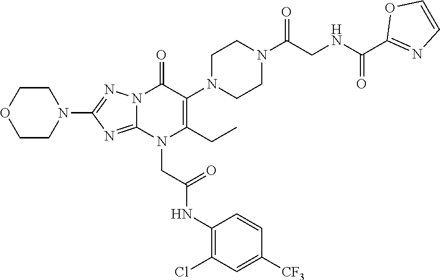 [SMILES: CCc1c(N2CCN(C(=O)CNC(=O)c3ncco3)CC2)c(=O)n2nc(N3CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide (Example 248 step 2) and oxazole-2-carboxylic acid to afford the title product as a white solid. LCMS calc. for C₃₀H₃₃ClF₃N₁₀O₆ [M+H]⁺: m/z=721.2; Found: 721.1.

### Example 250: N-(2-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)-2-oxoethyl)-2-fluoro-5-(methylamino)benzamide

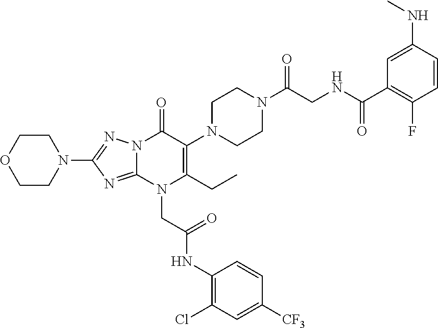 [SMILES: CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide (Example 248 step 2) and 2-fluoro-5-(methylamino)benzoic acid to afford the title product as a white solid. LCMS calc. for C₃₄H₃₈ClF₄N₁₀O₅ [M+H]⁺: m/z=776.3; Found: 776.2.

### Example 251: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)-4-fluoro-3-(methylamino)benzamide

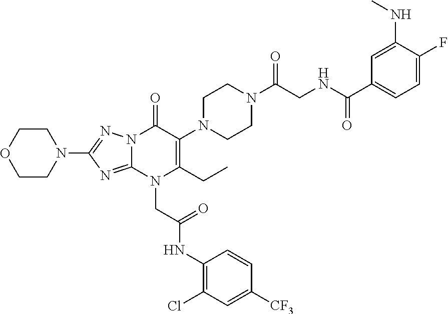 [SMILES: CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1Cl]

A mixture of 4-fluoro-3-(methylamino)benzoic acid (5.4 mg, 0.03 mmol), N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide (20 mg, 0.03 mmol, Example 248 step 2) and EDCI (9.2 mg, 0.05 mmol), Et₃N (16.2 mg, 0.16 mmol) and HOBt (6.5 mg, 0.05 mmol) in DMF (0.5 mL) was stirred at r.t. overnight. The reaction mixture was concentrated under reduced pressure, and the residue was purified by Prep-HPLC eluting with MeCN/H₂O (5-50%) to afford the title compound (4.1 mg, 36.5% yield) as a white solid. LCMS calc. for C₃₄H₃₈ClF₄N₁₀O₅ [M+H]⁺: m/z=777.3; Found: 777.2.

### Example 252: N-(2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-2-fluoro-3-hydroxybenzamide

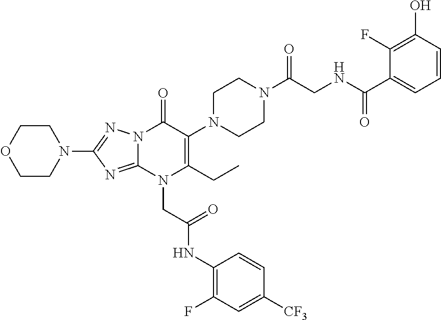 [SMILES: CCc1c(N2CCN(C(=O)CNC(=O)c3cccc(O)c3F)CC2)c(=O)n2nc(N3CCOCC3)nc2n1CC(=O)Nc1ccc(C(F)(F)F)cc1F]

### Step 1: tert-butyl (2-(4-(5-ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)carbamate

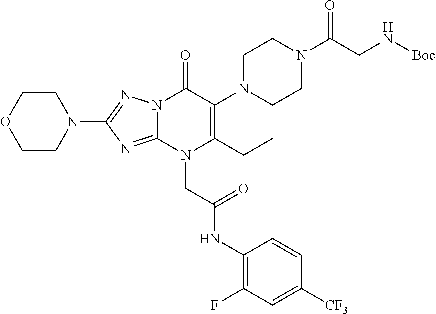 [SMILES: CCc1c(N2CCN(C(=O)CNC(=O)OC(C)(C)C)CC2)c(=O)n2nc(N3CCOCC3)nc2n1CC(=O)Nc1ccc(C(F)(F)F)cc1F]

This compound was prepared using procedures analogous to those described for method I using INT B23 and (tert-butoxycarbonyl)glycine to afford the title product as a white solid. LCMS calc. for C₃₁H₃₈F₄N₉O₆ [M−H]⁻: m/z=708.3; Found: [M−H]⁻708.2.

### Step 2: 2-(5-ethyl-6-(4-glycylpiperazin-1-yl)-2-morpholino-7-oxo-[1,2,4]triazolo[1,5-a]pyrimidin-4(7H)-yl)-N-(2-fluoro-4-(trifluoromethyl)phenyl)acetamide

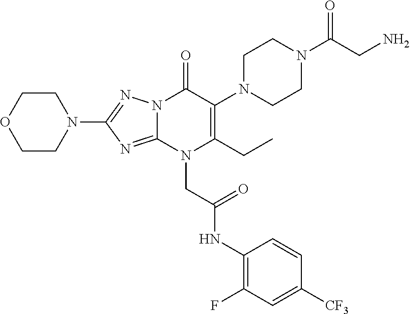 [SMILES: CCc1c(N2CCN(C(=O)CN)CC2)c(=O)n2nc(N3CCOCC3)nc2n1CC(=O)Nc1ccc(C(F)(F)F)cc1F]

To a mixture of tert-butyl (2-(4-(5-ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)carbamate (300 mg, 0.46 mmol) in DCM (3 mL) was added 4 M HCl/dioxane (3 mL). The mixture was stirred at r.t. for 2 h. The solvent was concentrated under reduced pressure to afford the title compound (150 mg, crude) as a brown solid. LCMS calc. for C₂₆H₃₂F₄N₉O₄ [M+H]⁺: m/z=610.2; Found: 610.2.

### Step 3: N-(2-(4-(5-ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-2-fluoro-3-hydroxybenzamide

This compound was prepared using procedures analogous to those described for method I using 2-(5-ethyl-6-(4-glycylpiperazin-1-yl)-2-morpholino-7-oxo-[1,2,4]triazolo[1,5-a]pyrimidin-4(7H)-yl)-N-(2-fluoro-4-(trifluoromethyl)phenyl)acetamide and 2-fluoro-3-hydroxybenzoic acid to afford the title product as a white solid. LCMS calc. for C₃₃H₃₅F₅N₉O₆ [M+H]⁺: m/z=748.3; Found: 748.1.

Example 253: N-(2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-3-fluoro-5-(methylamino)benzamide

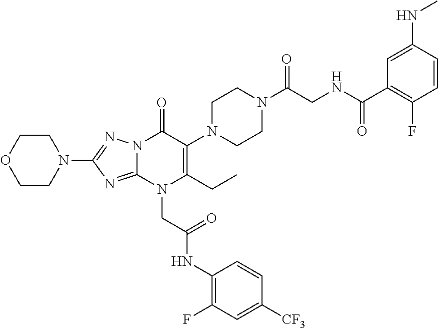 [SMILES: CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1F]

This compound was prepared using procedures analogous to those described for method I using 2-(5-ethyl-6-(4-glycylpiperazin-1-yl)-2-morpholino-7-oxo-[1,2,4]triazolo[1,5-a]pyrimidin-4(7H)-yl)-N-(2-fluoro-4-(trifluoromethyl)phenyl)acetamide (Example 252 step 2) and 2-fluoro-5-(methylamino)benzoic acid to afford the title product as a white solid. LCMS calc. for C₃₄H₃₆F₅N₁₀O₅ [M−H]⁻: m/z=759.3; Found: 759.2.

### Example 254: N-(2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-2-fluorobenzamide

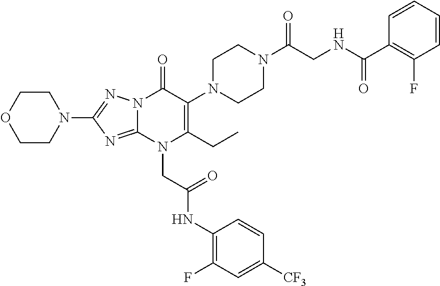 [SMILES: CCc1c(N2CCN(C(=O)CNC(=O)c3ccccc3F)CC2)c(=O)n2nc(N3CCOCC3)nc2n1CC(=O)Nc1ccc(C(F)(F)F)cc1F]

This compound was prepared using procedures analogous to those described for method I using INT B23 and (2-fluorobenzoyl)glycine to afford the title product as a white solid. LCMS calc. for C₃₃H₃₅F₅N₉O₅ [M+H]⁺: m/z=732.3; Found: 732.1.

### Example 255:2-Amino-N-(2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-3-fluorobenzamide

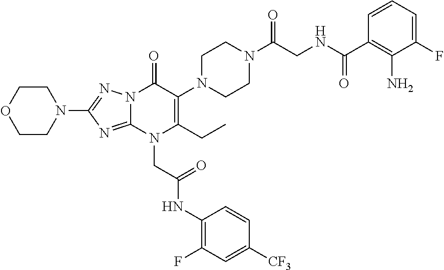 [SMILES: CCc1c(N2CCN(C(=O)CNC(=O)c3cccc(F)c3N)CC2)c(=O)n2nc(N3CCOCC3)nc2n1CC(=O)Nc1ccc(C(F)(F)F)cc1F]

This compound was prepared using procedures analogous to those described for method I using 2-(5-ethyl-6-(4-glycylpiperazin-1-yl)-2-morpholino-7-oxo-[1,2,4]triazolo[1,5-a]pyrimidin-4(7H)-yl)-N-(2-fluoro-4-(trifluoromethyl)phenyl)acetamide (Example 252 Step 2) and 2-amino-3-fluorobenzoic acid to afford the title product as a white solid. LCMS calc. for C₃₃H₃₆F₅N₁₀O₅ [M+H]⁺: m/z=747.3; Found: 747.2.

### Example 256: N-(2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxoethyl)-4-fluoro-3-(methylamino)benzamide

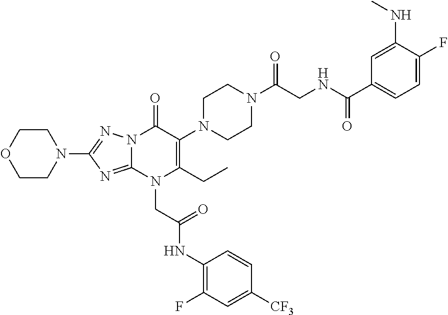 [SMILES: CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1F]

To a mixture of 4-fluoro-3-(methylamino)benzoic acid (5.4 mg, 0.03 mmol), 2-(5-ethyl-6-(4-glycylpiperazin-1-yl)-2-morpholino-7-oxo-[1,2,4]triazolo[1,5-a]pyrimidin-4(7H)-yl)-N-(2-fluoro-4-(trifluoromethyl)phenyl)acetamide (20 mg, 0.03 mmol, Example 252 Step 2) and EDCI (9.2 mg, 0.05 mmol) in DMF (0.5 mL) was added Et₃N (16.2 mg, 0.16 mmol) and HOBt (6.5 mg, 0.05 mmol). The mixture was stirred at r.t. overnight and purified by Prep-HPLC eluting with MeCN/H₂O (5-50%) to afford the title compound (8.9 mg, 35.7% yield) as a white solid. LCMS calc. for C₃₄H₃₈F₅N₁₀O₅ [M+H]⁺: m/z=761.3; Found: 761.2.

### Example 257: N-(2-(4-(5-Ethyl-4-(2-((2-fluoro-4-(trifluoromethyl)phenyl)amino)-2-oxoethyl)-2-(1,4-oxazepan-4-yl)-7-oxo-4,7-dihydro-[1,2,4]triazolo[1,5-a]pyrimidin-6-yl)piperazin-1-yl)-2-oxoethyl)oxazole-2-carboxamide

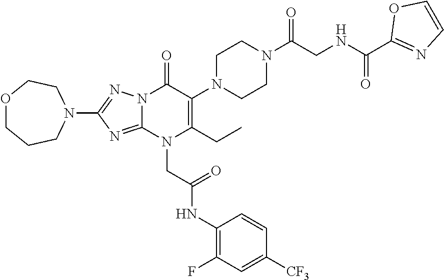 [SMILES: CCc1c(N2CCN(C(=O)CNC(=O)c3ncco3)CC2)c(=O)n2nc(N3CCCOCC3)nc2n1CC(=O)Nc1ccc(C(F)(F)F)cc1F]

### Step 1: tert-butyl (2-(4-(5-ethyl-4-(2-((2-fluoro-4-(trifluoromethyl)phenyl)amino)-2-oxoethyl)-2-(1,4-oxazepan-4-yl)-7-oxo-4,7-dihydro-[1,2,4]triazolo[1,5-a]pyrimidin-6-yl)piperazin-1-yl)-2-oxoethyl)carbamate

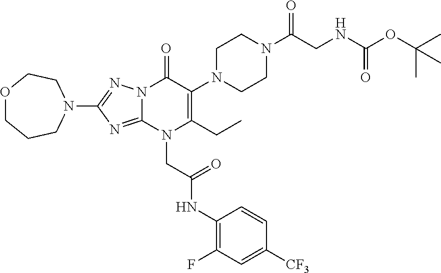 [SMILES: CCc1c(N2CCN(C(=O)CNC(=O)OC(C)(C)C)CC2)c(=O)n2nc(N3CCCOCC3)nc2n1CC(=O)Nc1ccc(C(F)(F)F)cc1F]

This compound was prepared using procedures analogous to those described for method I using INT B27 and (tert-butoxycarbonyl)glycine to afford the title compound (110 mg) as a white solid. LCMS calc. for C₃₂H₄₀F₄N₉O₆ [M−H]⁻: m/z=722.3; Found: 722.2.

### Step 2: 2-(5-ethyl-6-(4-glycylpiperazin-1-yl)-2-(1,4-oxazepan-4-yl)-7-oxo-[1,2,4]triazolo[1,5-a]pyrimidin-4(7H)-yl)-N-(2-fluoro-4-(trifluoromethyl)phenyl)acetamide

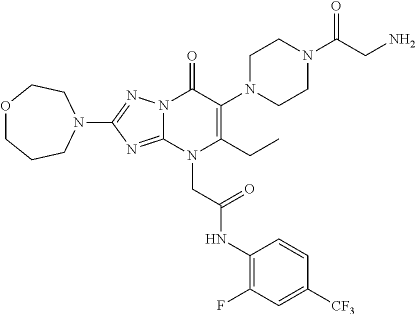 [SMILES: CCc1c(N2CCN(C(=O)CN)CC2)c(=O)n2nc(N3CCCOCC3)nc2n1CC(=O)Nc1ccc(C(F)(F)F)cc1F]

To a solution of tert-butyl (2-(4-(5-ethyl-4-(2-((2-fluoro-4-(trifluoromethyl)phenyl)amino)-2-oxoethyl)-2-(1,4-oxazepan-4-yl)-7-oxo-4,7-dihydro-[1,2,4]triazolo[1,5-a]pyrimidin-6-yl)piperazin-1-yl)-2-oxoethyl)carbamate (110 mg, 0.15 mmol) in DCM (2 mL) was added TFA (0.4 mL) at r.t. and was stirred for 12 h. The reaction mixture was concentrated under reduced pressure. The residue was purified by flash chromatography on a C18 column eluting with MeCN/H₂O (40-60%) to afford the title compound (80 mg) as an off-white solid. LCMS calc. for C₂₇H₃₂F₄N₉O₄ [M−H]⁻: m/z=622.3; Found: 622.2.

### Step 3: N-(2-(4-(5-ethyl-4-(2-((2-fluoro-4-(trifluoromethyl)phenyl)amino)-2-oxoethyl)-2-(1,4-oxazepan-4-yl)-7-oxo-4,7-dihydro-[1,2,4]triazolo[1,5-a]pyrimidin-6-yl)piperazin-1-yl)-2-oxoethyl)oxazole-2-carboxamide

This compound was prepared using procedures analogous to those described for method I using 2-(5-ethyl-6-(4-glycylpiperazin-1-yl)-2-(1,4-oxazepan-4-yl)-7-oxo-[1,2,4]triazolo[1,5-a]pyrimidin-4(7H)-yl)-N-(2-fluoro-4-(trifluoromethyl)phenyl)acetamide and oxazole-2-carboxylic acid to afford the title product as a white solid. LCMS calc. for C₃₁H₃₃F₄N₁₀O₆ [M−H]⁻: m/z=717.3; Found: 717.2.

### Example 258:2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxo-N-(thiazol-2-yl)acetamide

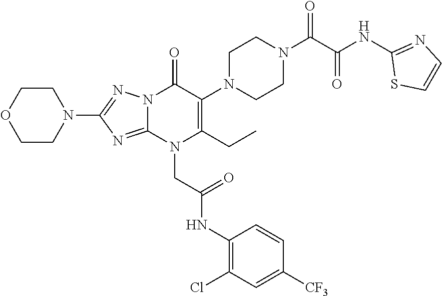 [SMILES: CCc1c(N2CCN(C(=O)C(=O)Nc3nccs3)CC2)c(=O)n2nc(N3CCOCC3)nc2n1CC(=O)Nc1ccc(C(F)(F)F)cc1Cl]

To a mixture of INT B4 (30 mg, 0.05 mmol) in DMF (1 mL) was added 2-oxo-2-(thiazol-2-ylamino)acetic acid (14 mg, 0.08 mmol), NMI (13 mg, 0.16 mmol), TCFH (22 mg, 0.08 mmol). The mixture was stirred at r.t. for 2 h., and then concentrated under reduced pressure. The residue was purified by Prep-HPLC on a C18 column eluting with MeCN/H₂O (60%) to afford the title compound (9 mg, 24% yield) as a white solid. LCMS calc. for C₂₉H₃₁ClF₃N₁₀O₅S [M+H]⁺: m/z=723.2; Found: 723.1.

### Example 259:2-(4-(4-(2-((2-Chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazin-1-yl)-2-oxo-N-(thiazol-2-yl)acetamide

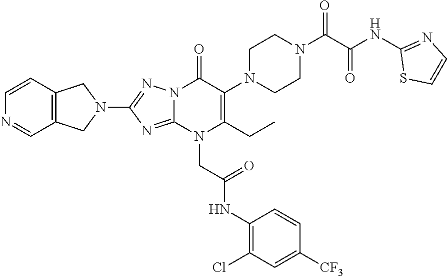 [SMILES: CCc1c(N2CCN(C(=O)C(=O)Nc3nccs3)CC2)c(=O)n2nc(N3Cc4ccncc4C3)nc2n1CC(=O)Nc1ccc(C(F)(F)F)cc1Cl]

To a mixture of INT B5 (30 mg, 0.05 mmol) in DMF (1 mL) was added 2-oxo-2-(thiazol-2-ylamino)acetic acid (13 mg, 0.07 mmol), NMI (12 mg, 0.15 mmol), TCFH (21 mg, 0.07 mmol). The mixture was stirred at r.t. for 2 h., and then concentrated under reduced pressure. The residue was purified by Prep-HPLC on a C18 column eluting with MeCN/H₂O (60%) to afford the title compound (14 mg, 37% yield) as a white solid. LCMS calc. for C₃₂H₃₀ClF₃N₁₁O₄S [M+H]⁺: m/z=756.2; Found: 756.1.

### Example 260:2-(4-(5-Ethyl-4-(2-((2-fluoro-4-(trifluoromethyl)phenyl)amino)-2-oxoethyl)-2-morpholino-7-oxo-4,7-dihydro-[1,2,4]triazolo[1,5-a]pyrimidin-6-yl)piperazin-1-yl)-2-oxo-N-(thiazol-2-yl)acetamide

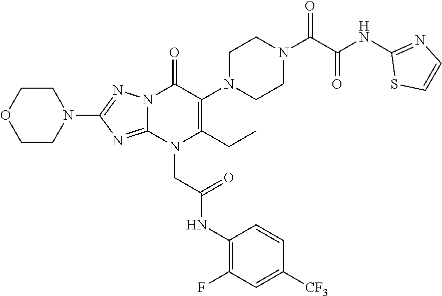 [SMILES: CCc1c(N2CCN(C(=O)C(=O)Nc3nccs3)CC2)c(=O)n2nc(N3CCOCC3)nc2n1CC(=O)Nc1ccc(C(F)(F)F)cc1F]

To a mixture of INT B23 (100 mg, 0.18 mmol) in DMF (1 mL) was added 2-oxo-2-(thiazol-2-ylamino)acetic acid (47 mg, 0.27 mmol), NMI (45 mg, 0.54 mmol), TCFH (76 mg, 0.27 mmol). The mixture was stirred at r.t. for 2 h., and then concentrated under reduced pressure. The residue was purified by Prep-HPLC on a C18 column eluting with MeCN/H₂O (50%) to afford the title compound (25.2 mg, 31% yield) as a white solid. LCMS calc. for C₂₉H₃₁F₄N₁₀O₅S [M+H]⁺: m/z=707.2; Found: 707.1.

### Example 261:2-(4-(2-(5,6-Dihydroimidazo[1,5-a]pyrazin-7 (8H)-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(thiazol-2-yl)acetamide

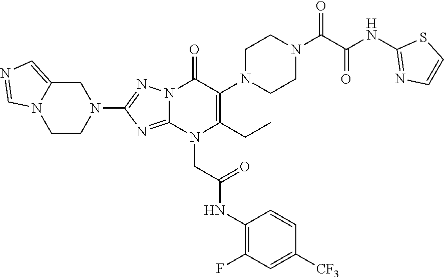 [SMILES: CCc1c(N2CCN(C(=O)C(=O)Nc3nccs3)CC2)c(=O)n2nc(N3CCn4cncc4C3)nc2n1CC(=O)Nc1ccc(C(F)(F)F)cc1F]

A mixture of 2-oxo-2-(thiazol-2-ylamino)acetic acid (10 mg, 0.06 mmol), INT B26 (18 mg, 0.03 mmol), EDCI (19 mg, 0.1 mmol), HOBt (13.5 mg, 0.1 mmol) and TEA (50 mg, 0.5 mmol) in DMF (1 mL) was stirred at r.t. overnight. The mixture was concentrated under reduced pressure. The residue was purified by Prep-HPLC on a C18 column eluting with MeCN/H₂O (35-50% with 0.1% NH₄HCO₃) to afford the title compound (10.5 mg) as a white solid. LCMS calc. for C₃₁H₂₉F₄N₁₂O₄S [M−H]⁻: m/z=741.2; Found: 741.3.

### Example 262:2-(4-(5-Ethyl-4-(2-((2-fluoro-4-(trifluoromethyl)phenyl)amino)-2-oxoethyl)-2-(1,4-oxazepan-4-yl)-7-oxo-4,7-dihydro-[1,2,4]triazolo[1,5-a]pyrimidin-6-yl)piperazin-1-yl)-2-oxo-N-(thiazol-2-yl)acetamide

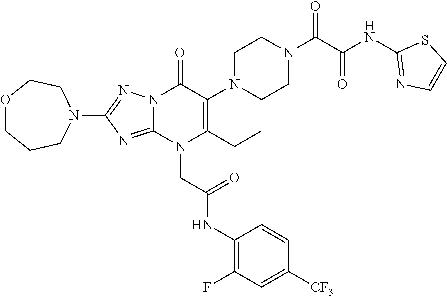 [SMILES: CCc1c(N2CCN(C(=O)C(=O)Nc3nccs3)CC2)c(=O)n2nc(N3CCCOCC3)nc2n1CC(=O)Nc1ccc(C(F)(F)F)cc1F]

A mixture of 2-oxo-2-(thiazol-2-ylamino)acetic acid (15 mg, 0.09 mmol), INT B27 (24 mg, 0.04 mmol), EDCI (19 mg, 0.1 mmol), HOBt (13.5 mg, 0.1 mmol) and TEA (50 mg, 0.5 mmol) in DMF (1 mL) was stirred at r.t. overnight. The mixture was concentrated under reduced pressure. The residue was purified by Prep-HPLC on a C18 column eluting with MeCN/H₂O (30-40% with 0.1% NH₄HCO₃) to afford the title compound (18.9 mg) as a white solid. LCMS calc. for C₃₀H₃₁F₄N₁₀O₅S [M−H]⁻: m/z=719.2; Found: 719.3.

### Example 263: N-(2-(4-(5-Ethyl-2-(1-methyl-4,6-dihydropyrrolo[3,4-c]pyrazol-5 (1H)-yl)-4-(2-((4-methylthiophen-2-yl)amino)-2-oxoethyl)-7-oxo-4,7-dihydro-[1,2,4]triazolo[1,5-a]pyrimidin-6-yl)piperazin-1-yl)-2-oxoethyl)-2-fluorobenzamide

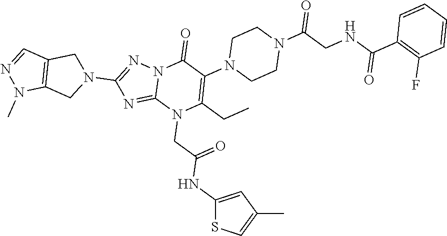 [SMILES: CCc1c(N2CCN(C(=O)CNC(=O)c3ccccc3F)CC2)c(=O)n2nc(N3Cc4cnn(C)c4C3)nc2n1CC(=O)Nc1cc(C)cs1]

This compound was prepared using procedures analogous to those described for method I using INT B28 and (2-fluorobenzoyl)glycine to afford the title product as a white solid. LCMS calc. for C₃₃H₃₇FN₁₁O₄S [M+H]⁺: m/z=702.3; Found: 702.2.

### Example 264: (R)-2-(4-(5-Ethyl-4-(2-((2-fluoro-4-(trifluoromethyl)phenyl)amino)-2-oxoethyl)-7-oxo-2-(3-oxotetrahydro-3H-oxazolo[3,4-a]pyrazin-7 (1H)-yl)-4,7-dihydro-[1,2,4]triazolo[1,5-a]pyrimidin-6-yl)piperazin-1-yl)-2-oxo-N-(thiazol-2-yl)acetamide

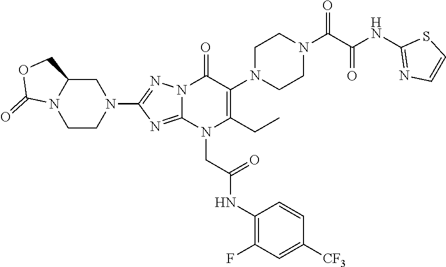 [SMILES: CCc1c(N2CCN(C(=O)C(=O)Nc3nccs3)CC2)c(=O)n2nc(N3CCN4C(=O)OC[C@H]4C3)nc2n1CC(=O)Nc1ccc(C(F)(F)F)cc1F]

A mixture of 2-oxo-2-(thiazol-2-ylamino)acetic acid (10 mg, 0.06 mmol), INT B24 (20 mg, 0.04 mmol), EDCI (19 mg, 0.1 mmol), HOBt (13.5 mg, 0.1 mmol) and TEA (50 mg, 0.5 mmol) in DMF (1 mL) was stirred at r.t. overnight. The mixture was concentrated under reduced pressure. The residue was purified by Prep-HPLC on a C18 column eluting with MeCN/H₂O (40-60% with 0.1% NH₄HCO₃) to afford the title compound (12.5 mg) as a white solid. LCMS calc. for C₃₁H₃₀F₄N₁₁O₆S [M−H]⁻: m/z=760.2; Found: 760.3.

### Example 265: N-((2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) carbamoyl)-2,5-difluorobenzamide

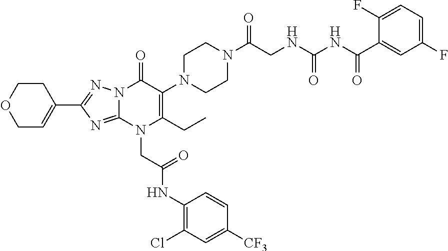 [SMILES: CCc1c(N2CCN(C(=O)CNC(=O)NC(=O)c3cc(F)ccc3F)CC2)c(=O)n2nc(C3=CCOCC3)nc2n1CC(=O)Nc1ccc(C(F)(F)F)cc1Cl]

A mixture of 2,5-difluorobenzoyl isocyanate (22 mg, 0.12 mmol), N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (40 mg, 0.06 mmol, Example 38) and TEA (24 mg, 0.12 mmol) in MeCN (1 mL) was stirred at r.t. overnight. The reaction mixture was concentrated under reduced pressure, and the residue was purified by Prep-HPLC on a C18 column eluting with MeCN/H₂O (40-60%, with 0.1% FA) to afford the title compound (11.2 mg, 23% yield) as a white solid. ¹H NMR (400 MHZ, DMSO-d₆) δ ppm 10.55 (s, 2H), 8.78 (d, J=4.8 Hz, 1H), 8.06 (d, J=8.6 Hz, 1H), 7.97 (d, J=2.0 Hz, 1H), 7.72 (dd, J=8.6, 2.1 Hz, 1H), 7.53 (ddd, J=8.4, 5.4, 3.1 Hz, 1H), 7.43 (ddq, J=18.3, 9.2, 3.9 Hz, 2H), 6.87-6.80 (m, 1H), 5.33 (s, 2H), 4.40 (d, J=12.5 Hz, 1H), 4.26 (q, J=2.7 Hz, 3H), 4.13 (dd, J=17.5, 4.7 Hz, 1H), 3.81 (q, J=5.9, 5.5 Hz, 3H), 3.55-3.44 (m, 2H), 3.22 (d, J=12.1 Hz, 2H), 3.05-2.94 (m, 2H), 2.83 (t, J=11.8 Hz, 2H), 2.73 (d, J=11.0 Hz, 2H), 1.20 (t, J=7.5 Hz, 3H). LCMS calc. for C₃₅H₃₄ClF₅N₉O₆ [M+H]⁺: m/z=806.2; Found: 806.2.

### Example 266: N-(2-Chloro-4-(trifluoromethyl)phenyl)-2-(2-(3,6-dihydro-2H-pyran-4-yl)-5-ethyl-6-(4-(4-hydroxyquinoline-3-carbonyl)piperazin-1-yl)-7-oxo-[1,2,4]triazolo[1,5-a]pyrimidin-4(7H)-yl)acetamide

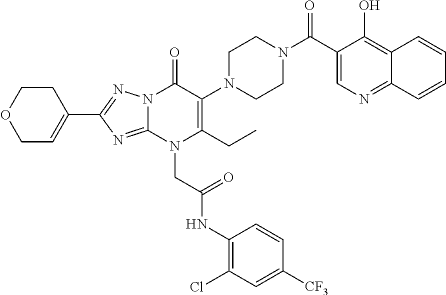 [SMILES: CCc1c(N2CCN(C(=O)c3cnc4ccccc4c3O)CC2)c(=O)n2nc(C3=CCOCC3)nc2n1CC(=O)Nc1ccc(C(F)(F)F)cc1Cl]

This compound was prepared by procedures analogous to those described for method A using 4-hydroxyquinoline-3-carboxylic acid to replace 4-(trifluoromethyl)-1H-imidazole-2-carboxylic acid to afford the title compound as a white solid. ¹H NMR (400 MHZ, DMSO-d₆) δ ppm 12.19 (s, 1H), 10.40 (s, 1H), 8.20 (s, 1H), 8.15 (dd, J=8.1, 1.2 Hz, 1H), 8.06 (d, J=8.5 Hz, 1H), 7.96 (d, J=1.6 Hz, 1H), 7.73-7.66 (m, 2H), 7.61 (d, J=8.1 Hz, 1H), 7.38 (dd, J=11.5, 4.5 Hz, 1H), 6.83 (s, 1H), 5.32 (s, 2H), 4.55 (d, J=10.9 Hz, 1H), 4.25 (d, J=2.4 Hz, 2H), 3.80 (t, J=5.4 Hz, 2H), 3.55-3.42 (m, 3H), 3.30 (s, 3H), 3.06-2.89 (m, 3H), 2.78 (d, J=13.6 Hz, 1H), 2.62 (d, J=10.7 Hz, 1H), 1.20 (t, J=7.5 Hz, 3H). LCMS calc. for C₃₅H₃₃ClF₃N₈O₅ [M+H]⁺: m/z=737.2; Found: 737.2.

### Example 267: N-(2-Chloro-4-(trifluoromethyl)phenyl)-2-(2-(3,6-dihydro-2H-pyran-4-yl)-5-ethyl-7-oxo-6-(4-((2-phenylacetyl)glycyl)piperazin-1-yl)-[1,2,4]triazolo[1,5-a]pyrimidin-4(7H)-yl)acetamide

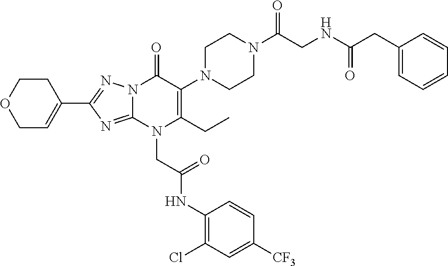 [SMILES: CCc1c(N2CCN(C(=O)CNC(=O)Cc3ccccc3)CC2)c(=O)n2nc(C3=CCOCC3)nc2n1CC(=O)Nc1ccc(C(F)(F)F)cc1Cl]

Method J: To a solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 48.0 μmol, Example 38), 2-phenylacetic acid (8 mg, 58 μmol) and HATU (23.8 mg, 63 μmol) in DMF (1 mL) was added DIPEA (25 mg, 193 μmol) at r.t. The mixture was stirred at r.t for 2 h., and concentrated. The residue was purified by Prep-HPLC on a C18 column eluting with MeCN/H₂O (5-95%, with 0.1% NH₄HCO₃) to afford the title product as a white solid. ¹H NMR (400 MHz, DMSO-d₆) δ ppm 10.35 (s, 1H), 8.17 (t, J=5.3 Hz, 1H), 8.06 (d, J=8.5 Hz, 1H), 7.97 (d, J=1.5 Hz, 1H), 7.72 (dd, J=8.7, 1.7 Hz, 1H), 7.31-7.27 (m, 4H), 7.21 (ddd, J=11.0, 5.5, 3.2 Hz, 1H), 6.83 (s, 1H), 5.32 (s, 2H), 4.37 (d, J=11.6 Hz, 1H), 4.25 (d, J=2.4 Hz, 2H), 4.08-3.97 (m, 2H), 3.81 (dd, J=11.5, 6.2 Hz, 3H), 3.52 (s, 2H), 3.49-3.41 (m, 1H), 3.37 (d, J=11.5 Hz, 1H), 3.18 (t, J=11.7 Hz, 2H), 2.98 (dd, J=14.5, 7.1 Hz, 2H), 2.82-2.64 (m, 4H), 1.18 (t, J=7.5 Hz, 3H). LCMS calc. for C₃₅H₃₇ClF₃N₈O₅ [M+H]⁺: m/z=741.2; Found: 741.2.

The following compounds listed in Table 11 were prepared by using an appropriate acid and Example 38 as the methods analogous to those described for method J.

TABLE 11
Preparations of Examples (Ex)

| | | | | LCMS |
|---|---|---|---|---|
| | | | | Cacl./ |
| Ex | Acid | Structure | Name | Found |
| 268 | 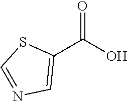 | 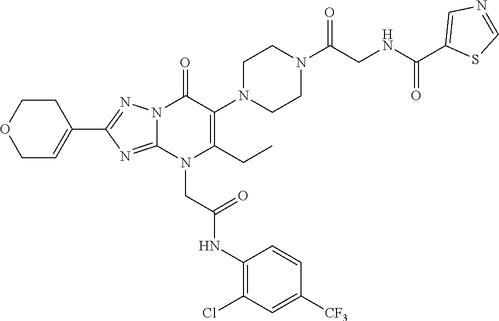 [SMILES: CCc1c(N2CCN(C(=O)CNC(=O)c3cncs3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)thiazole-5-carboxamide | [M + H]⁺: 734.2/ 734.1 |
| 269 | 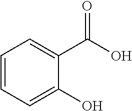 | 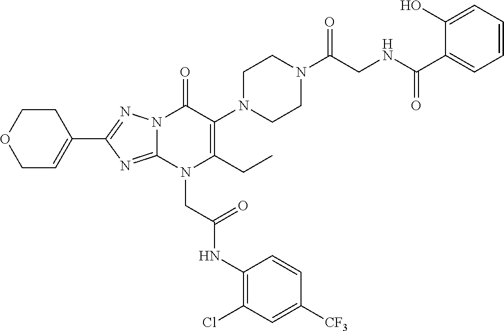 [SMILES: CCc1c(N2CCN(C(=O)CNC(=O)c3ccccc3O)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-hydroxybenzamide | [M + H]⁺: 743.2/ 743.2 |
| 270 | 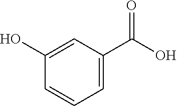 | 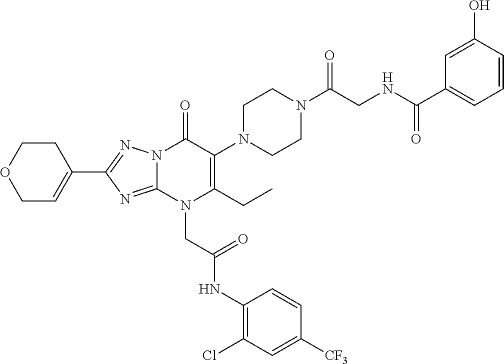 [SMILES: CCc1c(N2CCN(C(=O)CNC(=O)c3cccc(O)c3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-3-hydroxybenzamide | [M + H]⁺: 743.2/ 743.6 |
| 271 | 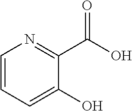 | 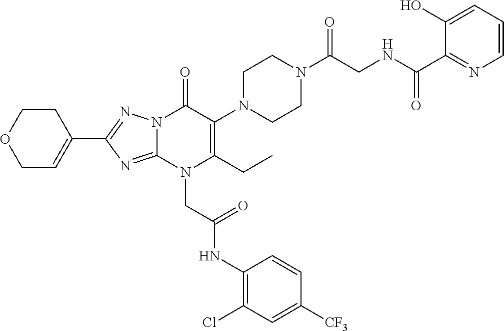 [SMILES: CCc1c(N2CCN(C(=O)CNC(=O)c3ncccc3O)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-3-hydroxypicolinamide | [M + H]⁺: 744.2/ 744.2 |
| 272 | 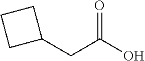 | 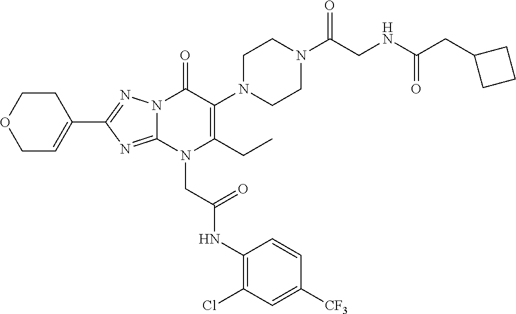 [SMILES: CCc1c(N2CCN(C(=O)CNC(=O)CC3CCC3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(6-(4- ((2- cyclobutylacetyl)glycyl)piperazin- 1-yl)-2-(3,6-dihydro-2H-pyran-4- yl)-5-ethyl-7-oxo- [1,2,4]triazolo[1,5-a]pyrimidin- 4(7H)-yl)acetamide | [M + H]⁺: 719.3/ 719.4 |
| 273 | 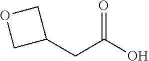 | 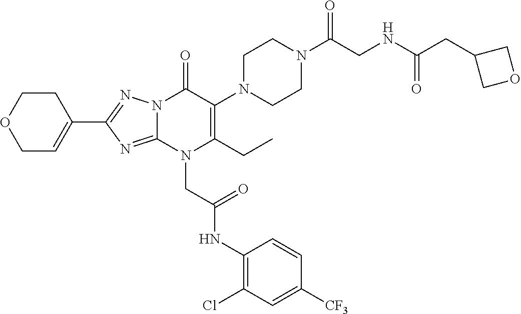 [SMILES: CCc1c(N2CCN(C(=O)CNC(=O)CC3COC3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4-((2-(oxetan-3- yl)acetyl)glycyl)piperazin-1-yl)-7- oxo-[1,2,4]triazolo[1,5- a]pyrimidin-4(7H)-yl)acetamide | [M + H]⁺: 721.2/ 721.2 |
| 274 | 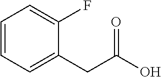 [SMILES: O=C(O)Cc1ccccc1F] | 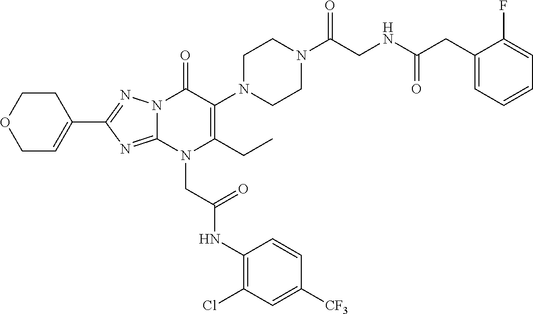 [SMILES: CCc1c(N2CCN(C(=O)CNC(=O)Cc3ccccc3F)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-6- (4-((2-(2- fluorophenyl)acetyl)glycyl)piperazin- 1-yl)-7-oxo-[1,2,4]triazolo[1,5- a]pyrimidin-4(7H)-yl)acetamide | [M + H]⁺: 759.2/ 759.2 |
| 275 | 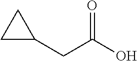 | 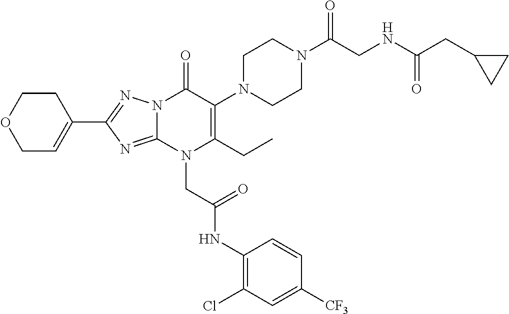 [SMILES: CCc1c(N2CCN(C(=O)CNC(=O)CC3CC3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(6-(4- ((2- cyclopropylacetyl)glycyl)piperazin- 1-yl)-2-(3,6-dihydro-2H-pyran-4- yl)-5-ethyl-7-oxo- [1,2,4]triazolo[1,5-a]pyrimidin- 4(7H)-yl)acetamide | [M + H]⁺: 705.2/ 705.5 |
| 276 | 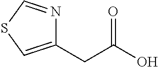 | 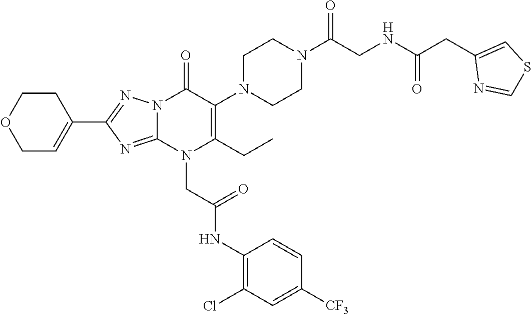 [SMILES: CCc1c(N2CCN(C(=O)CNC(=O)Cc3cscn3)CC2)c(=O)n2nc(C3=CCOCC3)nc2n1CC(=O)Nc1ccc(C(F)(F)F)cc1Cl] | N-(2-Chloro-4- (trifluoromethyl)phenyl)-2-(2-(3,6- dihydro-2H-pyran-4-yl)-5-ethyl-7- oxo-6-(4-((2-(thiazol-4- yl)acetyl)glycyl)piperazin-1-yl)- [1,2,4]triazolo[1,5-a]pyrimidin- 4(7H)-yl)acetamide | [M + H]⁺: 748.2/ 748.5 |
| 277 | 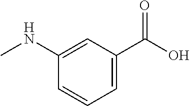 | 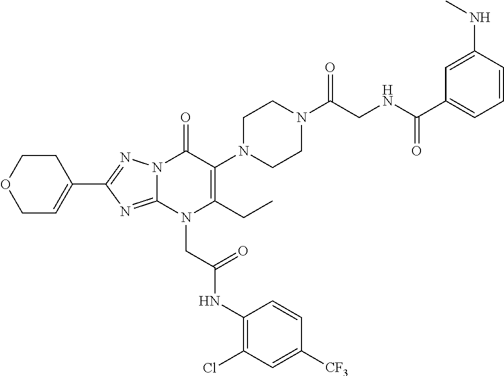 [SMILES: CCc1c(N2CCN(C(=O)CNC(=O)c3cccc(NC)c3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-3- (methylamino)benzamide | [M + H]⁺: 756.3/ 756.5 |
| 278 | 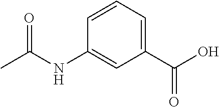 | 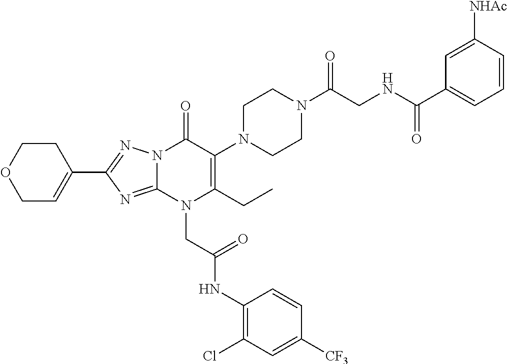 [SMILES: CCc1c(N2CCN(C(=O)CNC(=O)c3cccc(NC(C)=O)c3)CC2)c(=O)n2nc(C3=CCOCC3)nc2n1CC(=O)Nc1ccc(C(F)(F)F)cc1Cl] | 3-Acetamido-N-(2-(4-(4-(2-((2- chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)benzamide | [M + H]⁺: 784.3/ 784.3 |
| 279 | 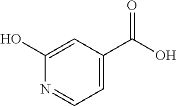 | 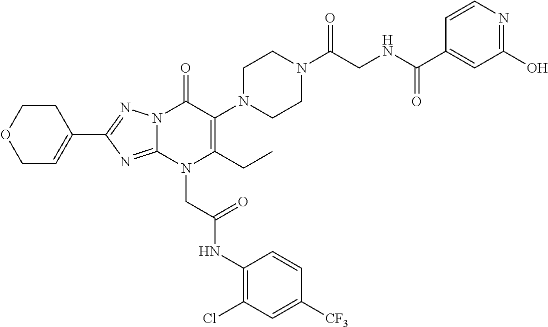 [SMILES: CCc1c(N2CCN(C(=O)CNC(=O)c3ccnc(O)c3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2- hydroxyisonicotinamide | [M + H]⁺: 744.2/ 744.2 |
| 280 | 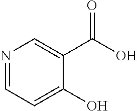 | 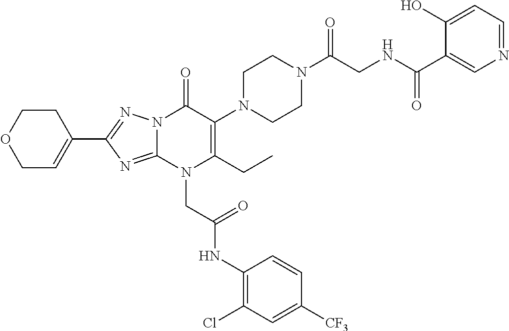 [SMILES: CCc1c(N2CCN(C(=O)CNC(=O)c3cnccc3O)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-4-hydroxynicotinamide | [M + H]⁺: 744.2/ 744.2 |
| 281 | 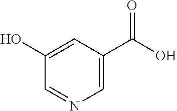 | 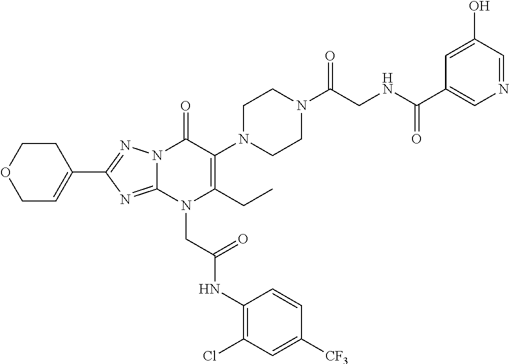 [SMILES: CCc1c(N2CCN(C(=O)CNC(=O)c3cncc(O)c3)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-5-hydroxynicotinamide | [M + H]⁺: 744.2/ 744.2 |
| 282 | 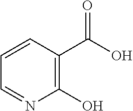 | 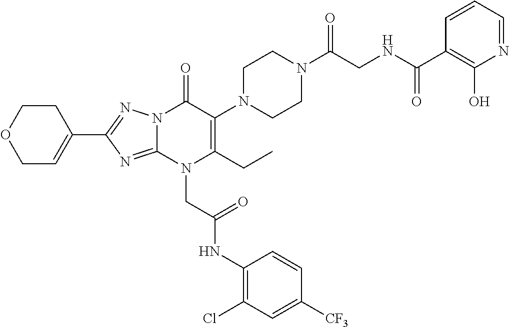 [SMILES: CCc1c(N2CCN(C(=O)CNC(=O)c3cccnc3O)CC2)c(=O)n2nc(C3=CCOCC3)nc2n1CC(=O)Nc1ccc(C(F)(F)F)cc1Cl] | N-(2-(4-(4-(2-((2-Chloro-4- (trifluoromethyl)phenyl)amino)-2- oxoethyl)-2-(3,6-dihydro-2H- pyran-4-yl)-5-ethyl-7-oxo-4,7- dihydro-[1,2,4]triazolo[1,5- a]pyrimidin-6-yl)piperazin-1-yl)-2- oxoethyl)-2-hydroxynicotinamide | [M + H]⁺: 744.2/ 744.6 |

TABLE 12
¹H NMR of Examples (Ex)

| Ex | ¹H NMR (400 MHz, DMSO-d₆) δ ppm |
|---|---|
| 268 | 10.38 (s, 1H), 9.25 (d, J = 0.7 Hz, 1H), 8.90 (t, J = 5.7 Hz, 1H), 8.54 (s, 1H), 8.06 (d, J = 8.6 Hz, 1H), |
| | 7.97 (d, J = 1.8 Hz, 1H), 7.75-7.69 (m, 1H), 6.83 (dd, J = 3.3, 1.7 Hz, 1H), 5.33 (s, 2H), 4.38 (d, |
| | J = 12.7 Hz, 1H), 4.26 (d, J = 3.2 Hz, 2H), 4.20 (t, J = 5.3 Hz, 2H), 3.94 (d, J = 12.7 Hz, 1H), 3.81 (t, J = 5.5 |
| | Hz, 2H), 3.50 (d, J = 11.7 Hz, 2H), 3.25 (s, 2H), 3.00 (d, J = 7.8 Hz, 2H), 2.86-2.65 (m, 4H), 1.20 (t, |
| | J = 7.4 Hz, 3H). |
| 269 | 12.12 (s, 1H), 10.36 (s, 1H), 9.07 (t, J = 5.3 Hz, 1H), 8.06 (d, J = 8.5 Hz, 1H), 7.97 (d, J = 2.1 Hz, 1H), |
| | 7.91 (dd, J = 7.9, 1.7 Hz, 1H), 7.72 (dd, J = 8.8, 2.2 Hz, 1H), 7.44-7.38 (m, 1H), 6.92 (t, J = 8.4 Hz, |
| | 2H), 6.83 (s, 1H), 5.33 (s, 2H), 4.44-4.37 (m, 1H), 4.26 (q, J = 5.3, 4.3 Hz, 4H), 3.92 (d, J = 12.8 Hz, |
| | 1H), 3.80 (t, J = 5.5 Hz, 2H), 3.57-3.36 (m, 4H), 3.25 (d, J = 12.6 Hz, 2H), 3.00 (d, J = 7.7 Hz, 2H), |
| | 2.73 (d, J = 11.1 Hz, 2H), 1.20 (t, J = 7.5 Hz, 3H). |
| 270 | 10.36 (s, 1H), 9.65 (s, 1H), 8.45 (t, J = 5.6 Hz, 1H), 8.06 (d, J = 8.5 Hz, 1H), 7.97 (d, J = 1.5 Hz, 1H), |
| | 7.73 (dd, J = 8.8, 1.7 Hz, 1H), 7.29 (ddd, J = 7.3, 6.1, 1.8 Hz, 3H), 6.94-6.90 (m, 1H), 6.84 (s, 1H), |
| | 5.33 (s, 2H), 4.39 (d, J = 12.5 Hz, 1H), 4.26 (d, J = 2.4 Hz, 2H), 4.16 (d, J = 5.7 Hz, 2H), 3.96 (d, |
| | J = 12.4 Hz, 1H), 3.81 (t, J = 5.4 Hz, 2H), 3.51 (t, J = 10.9 Hz, 1H), 3.43-3.36 (m, 2H), 3.28-3.21 (m, |
| | 1H), 3.00 (d, J = 7.4 Hz, 2H), 2.85-2.70 (m, 4H), 1.20 (t, J = 7.5 Hz, 3H). |
| 271 | 12.37 (s, 1H), 10.36 (s, 1H), 9.12 (t, J = 5.5 Hz, 1H), 8.21 (dd, J = 4.4, 1.4 Hz, 1H), 8.07 (d, J = 8.5 Hz, |
| | 1H), 7.99-7.95 (m, 1H), 7.75-7.70 (m, 1H), 7.56 (dd, J = 8.5, 4.4 Hz, 1H), 7.44 (dd, J = 8.5, 1.4 Hz, |
| | 1H), 6.87-6.79 (m, 1H), 5.33 (s, 2H), 4.43-4.30 (m, 2H), 4.25 (q, J = 3.0 Hz, 2H), 4.24-4.16 (m, |
| | 1H), 3.91 (d, J = 12.6 Hz, 1H), 3.80 (t, J = 5.4 Hz, 2H), 3.46 (dt, J = 49.2, 11.6 Hz, 4H), 3.31-3.20 (m, |
| | 2H), 3.00 (q, J = 7.4 Hz, 2H), 2.74 (d, J = 11.1 Hz, 2H), 1.20 (t, J = 7.5 Hz, 3H). |
| 272 | 10.37 (s, 1H), 8.06 (d, J = 8.5 Hz, 1H), 7.96 (d, J = 1.2 Hz, 1H), 7.87 (t, J = 5.5 Hz, 1H), 7.74-7.70 (m, |
| | 1H), 6.83 (s, 1H), 5.31 (s, 2H), 4.37 (d, J = 11.0 Hz, 1H), 4.25 (d, J = 2.4 Hz, 2H), 3.97 (dd, J = 12.9, 5.4 |
| | Hz, 2H), 3.86 (s, 1H), 3.80 (t, J = 5.4 Hz, 2H), 3.50-3.40 (m, 2H), 3.37 (d, J = 11.7 Hz, 2H), 3.19 (t, |
| | J = 11.5 Hz, 1H), 2.98 (d, J = 7.4 Hz, 2H), 2.70 (d, J = 11.7 Hz, 2H), 2.59 (dd, J = 15.6, 7.8 Hz, 2H), 2.27 |
| | (d, J = 7.5 Hz, 2H), 2.05-1.98 (m, 2H), 1.83-1.76 (m, 2H), 1.73-1.64 (m, 2H), 1.19 (t, J = 7.4 Hz, |
| | 3H). |
| 273 | 10.36 (s, 1H), 8.10-8.03 (m, 2H), 7.97 (s, 1H), 7.72 (d, J = 9.0 Hz, 1H), 6.83 (s, 1H), 5.32 (s, 2H), 4.64 |
| | (dd, J = 7.8, 5.9 Hz, 2H), 4.41-4.28 (m, 3H), 4.25 (d, J = 2.5 Hz, 2H), 4.08-3.88 (m, 2H), 3.81 (dd, |
| | J = 13.9, 8.6 Hz, 3H), 3.44 (d, J = 11.8 Hz, 1H), 3.35 (s, 2H), 3.20 (td, J = 14.3, 7.2 Hz, 3H), 2.98 (d, |
| | J = 7.6 Hz, 2H), 2.76 (s, 1H), 2.71 (s, 2H), 2.57 (d, J = 7.8 Hz, 2H), 1.19 (t, J = 7.5 Hz, 3H). |
| 274 | 10.36 (s, 1H), 8.18 (t, J = 5.4 Hz, 1H), 8.06 (d, J = 8.5 Hz, 1H), 7.97 (d, J = 1.6 Hz, 1H), 7.72 (dd, |
| | J = 8.7, 1.6 Hz, 1H), 7.39 (dd, J = 8.4, 6.6 Hz, 1H), 7.29 (ddd, J = 7.2, 6.4, 1.8 Hz, 1H), 7.17-7.12 (m, 2H), |
| | 6.83 (s, 1H), 5.32 (s, 2H), 4.38 (d, J = 12.5 Hz, 1H), 4.25 (d, J = 2.4 Hz, 2H), 4.03 (dd, J = 15.0, 5.3 Hz, |
| | 2H), 3.85 (s, 1H), 3.80 (t, J = 5.4 Hz, 2H), 3.58 (s, 2H), 3.51-3.42 (m, 2H), 3.37 (s, 2H), 3.20 (t, |
| | J = 11.2 Hz, 1H), 2.98 (d, J = 7.4 Hz, 2H), 2.79 (d, J = 11.6 Hz, 1H), 2.75-2.69 (m, 2H), 1.19 (t, J = 7.4 |
| | Hz, 3H). |
| 275 | 10.36 (s, 1H), 8.06 (d, J = 8.5 Hz, 1H), 7.97 (d, J = 1.6 Hz, 1H), 7.86 (t, J = 5.4 Hz, 1H), 7.72 (dd, |
| | J = 8.7, 1.7 Hz, 1H), 6.83 (s, 1H), 5.32 (s, 2H), 4.38 (d, J = 11.7 Hz, 1H), 4.25 (d, J = 2.5 Hz, 2H), 4.00 (dd, |
| | J = 17.8, 5.3 Hz, 2H), 3.85 (d, J = 12.5 Hz, 1H), 3.80 (t, J = 5.4 Hz, 2H), 3.45 (dd, J = 26.9, 16.4 Hz, |
| | 4H), 3.21 (t, J = 11.7 Hz, 1H), 2.99 (q, J = 7.0 Hz, 2H), 2.79 (d, J = 12.5 Hz, 1H), 2.71 (d, J = 10.3 Hz, |
| | 2H), 2.08 (d, J = 6.9 Hz, 2H), 1.19 (t, J = 7.5 Hz, 3H), 1.01-0.92 (m, 1H), 0.47-0.42 (m, 2H), 0.17- |
| | 0.13 (m, 2H). |
| 276 | 10.36 (s, 1H), 9.04 (d, J = 1.9 Hz, 1H), 8.16 (t, J = 5.3 Hz, 1H), 8.06 (d, J = 8.5 Hz, 1H), 7.97 (d, J = 1.6 |
| | Hz, 1H), 7.72 (dd, J = 8.8, 1.8 Hz, 1H), 7.55-7.53 (m, 1H), 6.83 (s, 1H), 5.32 (s, 2H), 4.38 (d, J = 12.0 |
| | Hz, 1H), 4.25 (d, J = 2.5 Hz, 2H), 4.11-3.97 (m, 2H), 3.85 (s, 1H), 3.80 (t, J = 5.4 Hz, 2H), 3.74 (s, |
| | 2H), 3.47 (t, J = 11.0 Hz, 1H), 3.39 (s, 2H), 3.34-3.29 (m, 1H), 3.20 (t, J = 11.5 Hz, 1H), 2.98 (q, |
| | J = 7.2 Hz, 2H), 2.79 (d, J = 11.7 Hz, 1H), 2.70 (s, 2H), 1.19 (t, J = 7.5 Hz, 3H). |
| 277 | 10.35 (s, 1H), 8.35 (t, J = 5.6 Hz, 1H), 8.06 (d, J = 8.4 Hz, 1H), 7.97 (d, J = 1.5 Hz, 1H), 7.72 (dd, |
| | J = 8.7, 1.6 Hz, 1H), 7.16 (t, J = 7.8 Hz, 1H), 7.03 (t, J = 4.3 Hz, 2H), 6.85-6.82 (m, 1H), 6.70-6.67 (m, |
| | 1H), 5.83 (q, J = 5.0 Hz, 1H), 5.32 (s, 2H), 4.39 (d, J = 12.3 Hz, 1H), 4.26 (d, J = 2.5 Hz, 2H), 4.16 (d, |
| | J = 5.7 Hz, 2H), 3.96 (d, J = 12.5 Hz, 1H), 3.81 (t, J = 5.4 Hz, 2H), 3.51 (t, J = 10.6 Hz, 1H), 3.44-3.34 |
| | (m, 2H), 3.30-3.20 (m, 2H), 3.00 (q, J = 7.2 Hz, 2H), 2.81 (d, J = 10.7 Hz, 1H), 2.74 (s, 1H), 2.71 (d, |
| | J = 5.0 Hz, 4H), 1.20 (t, J = 7.5 Hz, 3H). |
| 278 | 10.36 (s, 1H), 10.10 (s, 1H), 8.49 (t, J = 5.6 Hz, 1H), 8.08-8.01 (m, 2H), 7.97 (d, J = 1.6 Hz, 1H), 7.97 |
| | (dd, J = 8.1, 1.1 Hz, 1H), 7.72 (dd, J = 8.8, 1.7 Hz, 1H), 7.54 (d, J = 7.9 Hz, 1H), 7.39 (t, J = 7.9 Hz, 1H), |
| | 6.83 (s, 1H), 5.33 (s, 2H), 4.39 (d, J = 11.9 Hz, 1H), 4.25 (d, J = 2.4 Hz, 2H), 4.18 (d, J = 5.6 Hz, 2H), |
| | 3.96 (d, J = 12.3 Hz, 1H), 3.80 (t, J = 5.4 Hz, 2H), 3.52 (t, J = 10.6 Hz, 1H), 3.40 (d, J = 11.6 Hz, 2H), |
| | 3.24 (t, J = 11.7 Hz, 2H), 3.00 (dd, J = 14.3, 6.9 Hz, 2H), 2.80 (t, J = 12.2 Hz, 1H), 2.73 (d, J = 10.9 Hz, |
| | 2H), 2.07 (d, J = 5.0 Hz, 3H), 1.20 (t, J = 7.5 Hz, 3H). |
| 279 | 11.83 (s, 1H), 10.36 (s, 1H), 8.74 (t, J = 5.8 Hz, 1H), 8.06 (d, J = 8.6 Hz, 1H), 7.97 (s, 1H), 7.72 (d, |
| | J = 7.2 Hz, 1H), 7.47 (d, J = 6.6 Hz, 1H), 6.83 (s, 1H), 6.76 (s, 1H), 6.55-6.45 (m, 1H), 5.32 (s, 2H), 4.37 (d, |
| | J = 12.0 Hz, 1H), 4.25 (d, J = 2.1 Hz, 2H), 4.17-4.12 (m, 2H), 3.93 (d, J = 12.5 Hz, 1H), 3.80 (t, J = 5.3 |
| | Hz, 2H), 3.50 (t, J = 11.1 Hz, 1H), 3.38 (d, J = 10.2 Hz, 1H), 3.26 (dd, J = 21.0, 10.8 Hz, 2H), 2.99 (d, |
| | J = 7.5 Hz, 2H), 2.75 (dt, J = 26.0, 13.0 Hz, 4H), 1.20 (t, J = 7.4 Hz, 3H). |
| 280 | 10.64 (s, 1H), 8.43 (s, 1H), 8.06 (d, J = 8.6 Hz, 1H), 7.97 (s, 1H), 7.79 (d, J = 7.2 Hz, 1H), 7.72 (d, |
| | J = 7.3 Hz, 1H), 6.83 (s, 1H), 6.40 (d, J = 7.2 Hz, 1H), 5.32 (s, 2H), 4.44-4.29 (m, 2H), 4.22 (dd, J = 18.2, |
| | 13.4 Hz, 3H), 3.85 (d, J = 12.8 Hz, 1H), 3.80 (t, J = 5.3 Hz, 2H), 3.50 (d, J = 9.0 Hz, 1H), 3.45 (s, 2H), |
| | 3.23 (s, 1H), 2.99 (d, J = 7.4 Hz, 2H), 2.75 (dt, J = 25.6, 12.9 Hz, 4H), 1.20 (t, J = 7.5 Hz, 3H). |
| 281 | 10.30 (d, J = 40.5 Hz, 2H), 8.74 (s, 1H), 8.52 (d, J = 1.6 Hz, 1H), 8.26 (d, J = 2.7 Hz, 1H), 8.06 (d, |
| | J = 8.5 Hz, 1H), 7.97 (s, 1H), 7.72 (d, J = 7.0 Hz, 1H), 7.58-7.54 (m, 1H), 6.83 (s, 1H), 5.32 (s, 2H), 4.39 (d, |
| | J = 11.6 Hz, 1H), 4.26 (d, J = 2.5 Hz, 2H), 4.20 (s, 2H), 3.96 (d, J = 13.0 Hz, 1H), 3.80 (t, J = 5.4 Hz, |
| | 2H), 3.52 (t, J = 12.0 Hz, 1H), 3.39 (d, J = 10.0 Hz, 2H), 3.24 (d, J = 11.8 Hz, 1H), 3.00 (d, J = 7.2 Hz, |
| | 2H), 2.75 (dt, J = 28.6, 14.9 Hz, 4H), 1.20 (t, J = 7.4 Hz, 3H). |
| 282 | 12.49 (s, 1H), 10.37 (s, 1H), 10.16 (t, J = 4.8 Hz, 1H), 8.34 (dd, J = 7.1, 2.2 Hz, 1H), 8.07 (d, J = 8.5 Hz, |
| | 1H), 7.98 (d, J = 1.5 Hz, 1H), 7.73 (dd, J = 8.6, 1.7 Hz, 2H), 6.83 (s, 1H), 6.49-6.45 (m, 1H), 5.33 (s, |
| | 2H), 4.47-4.34 (m, 2H), 4.32 (d, J = 4.5 Hz, 1H), 4.26 (d, J = 2.4 Hz, 2H), 4.21 (d, J = 12.7 Hz, 1H), |
| | 3.85 (d, J = 13.7 Hz, 1H), 3.80 (t, J = 5.4 Hz, 2H), 3.50 (t, J = 10.8 Hz, 1H), 3.40 (d, J = 11.5 Hz, 2H), |
| | 3.24 (t, J = 11.2 Hz, 1H), 2.99 (dd, J = 14.2, 6.8 Hz, 2H), 2.81 (t, J = 12.2 Hz, 1H), 2.73 (d, J = 10.6 Hz, |
| | 2H), 1.20 (t, J = 7.4 Hz, 3H). |

Example 283: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-1-methyl-3-oxo-2,3-dihydro-1H-pyrazole-4-carboxamide

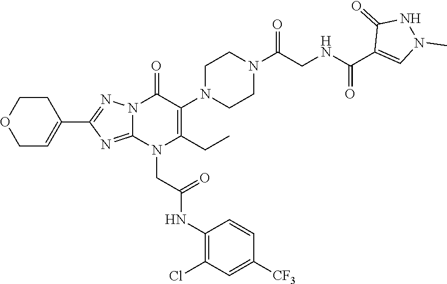 [SMILES: CCc1c(N2CCN(C(=O)CNC(=O)c3cn(C)[nH]c3=O)CC2)c(=O)n2nc(C3=CCOCC3)nc2n1CC(=O)Nc1ccc(C(F)(F)F)cc1Cl]

This compound was prepared by the procedure analogous to that described for method J using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and INT C26 to afford the title product as a white solid. ¹H NMR (400 MHZ, DMSO-d₆) δ ppm 11.11 (s, 1H), 10.36 (s, 1H), 8.06 (d, J=8.4 Hz, 1H), 7.97 (s, 1H), 7.86 (s, 1H), 7.73 (t, J=7.3 Hz, 2H), 6.83 (s, 1H), 5.32 (s, 2H), 4.39 (m, 1H), 4.25 (d, J=2.3 Hz, 2H), 4.22-4.06 (m, 2H), 3.83-3.78 (m, 3H), 3.65 (s, 3H), 3.54-3.34 (m, 4H), 3.28-3.16 (m, 2H), 2.99 (d, J=7.3 Hz, 2H), 2.72 (d, J=8.0 Hz, 2H), 1.19 (dd, J=9.5, 5.4 Hz, 3H). LCMS calc. for C₃₂H₃₅ClF₃N₁₀O₆ [M+H]⁺: m/z=747.2; Found: 747.2.

### Example 284: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoro-5-(methylamino)benzamide

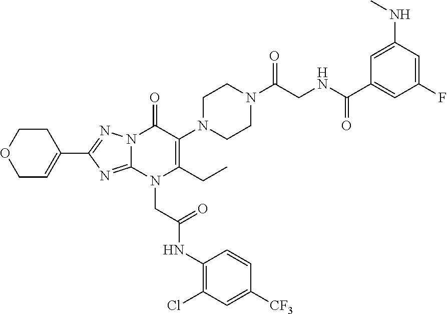 [SMILES: CCc1c(N2CCN(C(=O)CNC(=O)c3cc(F)cc(NC)c3)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 3-fluoro-5-(methylamino)benzoic acid to afford the title product as a white solid. LCMS calc. for C₃₅H₃₅ClF₄N₉O₅ [M−H]⁻: m/z=772.2; Found: 772.1.

### Example 285: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-fluoro-3-(methylamino)benzamide

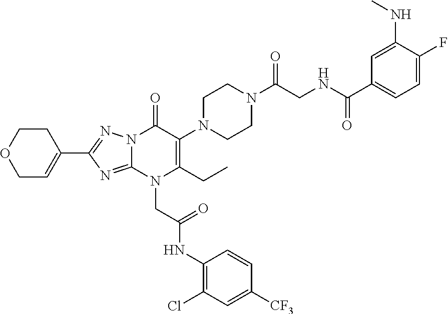 [SMILES: CCc1c(N2CCN(C(=O)CNC(=O)c3ccc(F)c(NC)c3)CC2)c(=O)n2nc(C3=CCOCC3)nc2n1CC(=O)Nc1ccc(C(F)(F)F)cc1Cl]

To a mixture of 4-fluoro-3-(methylamino)benzoic acid (12 mg, 0.07 mmol), N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (43.9 mg, 0.07 mmol, Example 38) and EDCI (20.1 mg, 0.11 mmol) in DMF (0.5 mL) was added Et₃N (45.1 mg, 0.35 mmol) and HOBt (14.2 mg, 0.11 mmol). The mixture was stirred at r.t. overnight. The reaction mixture was purified by Prep-HPLC eluting with MeCN/H₂O (5-35%) to afford the title compound (19.4 mg, 35% yield) as a white solid. LCMS calc. for C₃₅H₃₇ClF₄N₉O₅ [M+H]⁺: m/z=774.3; Found: 774.2.

### Example 286: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-(dimethylamino)-4-fluorobenzamide

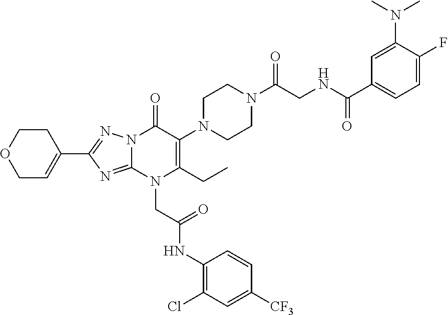 [SMILES: CCc1c(N2CCN(C(=O)CNC(=O)c3ccc(F)c(N(C)C)c3)CC2)c(=O)n2nc(C3=CCOCC3)nc2n1CC(=O)Nc1ccc(C(F)(F)F)cc1Cl]

This compound was prepared using procedures analogous to those described for Example 285 using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 3-(dimethylamino)-4-fluorobenzoic acid to afford the title product as a white solid. LCMS calc. for C₃₆H₃₉ClF₄N₉O₅ [M+H]⁺: m/z=788.3; Found: 788.2.

### Example 287: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-3-(methylamino)benzamide

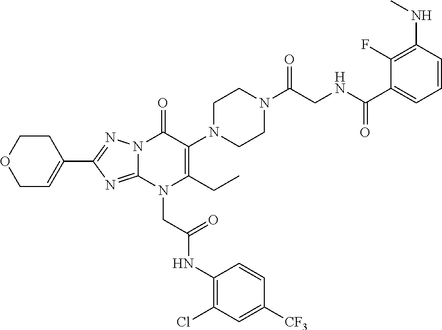 [SMILES: CCc1c(N2CCN(C(=O)CNC(=O)c3cccc(NC)c3F)CC2)c(=O)n2nc(C3=CCOCC3)nc2n1CC(=O)Nc1ccc(C(F)(F)F)cc1Cl]

This compound was prepared using procedures analogous to those described for Example 285 using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-fluoro-3-(methylamino)benzoic acid to afford the title product as a white solid. LCMS calc. for C₃₅H₃₇ClF₄N₉O₅ [M+H]⁺: m/z=774.3; Found: 774.2.

Example 288:3-Amino-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzamide

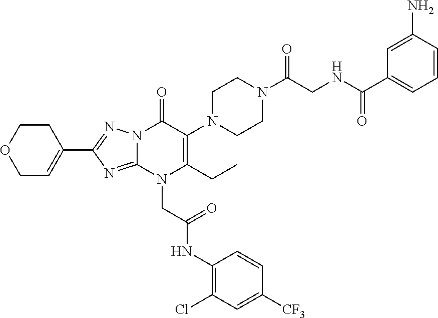 [SMILES: CCc1c(N2CCN(C(=O)CNC(=O)c3cccc(N)c3)CC2)c(=O)n2nc(C3=CCOCC3)nc2n1CC(=O)Nc1ccc(C(F)(F)F)cc1Cl]

### Step 1: tert-butyl (3-((2-(4-(4-(2-((2-chloro-4-(trifluoromethyl)phenyl)amino)-2-oxo ethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) carbamoyl)phenyl)carbamate

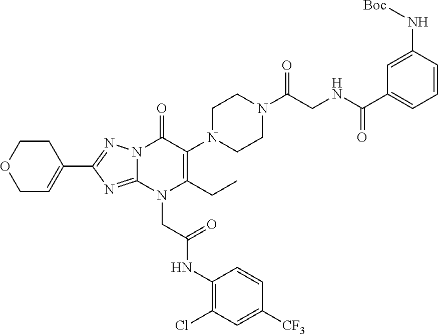 [SMILES: CCc1c(N2CCN(C(=O)CNC(=O)c3cccc(NC(=O)OC(C)(C)C)c3)CC2)c(=O)n2nc(C3=CCOCC3)nc2n1CC(=O)Nc1ccc(C(F)(F)F)cc1Cl]

This compound was prepared by the procedure analogous to that described for method J using 3-((tert-butoxycarbonyl)amino)benzoic acid to replace 2-phenylacetic acid to afford the title product as a white solid. LCMS calc. for C₃₉H₄₄ClF₃N₉O₇ [M+H]⁺: m/z=842.3; Found: 842.5.

### Step 2: 3-amino-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzamide

To a solution of tert-butyl (3-((2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro[1,2,4]triazolo[1,5-a]pyrimidin-6-yl)piperazin-1-yl)-2-oxoethyl) carbamoyl)phenyl)carbamate (21 mg, 0.05 mmol) in DCM (1 mL) was added 4 M HCl/dioxane (2 mL). The mixture was stirred at r.t. for 1 h., and concentrated under reduced pressure. The residue was purified by Prep-HPLC on a C18 column eluting with MeOH/H₂O (5-95%, with 0.1% FA) to afford the title compound (10.83 mg, 58% yield) as a white solid. ¹H NMR (400 MHZ, DMSO-d₆) δ ppm 10.37 (s, 1H), 8.25 (t, J=5.6 Hz, 1H), 8.06 (d, J=8.4 Hz, 1H), 7.97 (d, J=1.5 Hz, 1H), 7.72 (dd, J=8.7, 1.6 Hz, 1H), 7.10 (d, J=7.7 Hz, 1H), 7.07 (dd, J=3.5, 1.4 Hz, 1H), 6.99 (d, J=7.8 Hz, 1H), 6.84 (s, 1H), 6.70 (dd, J=7.9, 1.4 Hz, 1H), 5.32 (s, 2H), 5.25 (s, 2H), 4.39 (d, J=12.0 Hz, 1H), 4.26 (d, J=2.4 Hz, 2H), 4.15 (d, J=5.5 Hz, 2H), 3.95 (d, J=12.7 Hz, 1H), 3.81 (t, J=5.4 Hz, 2H), 3.51 (t, J=10.7 Hz, 1H), 3.40 (d, J=11.6 Hz, 2H), 3.30-3.18 (m, 2H), 3.00 (d, J=7.5 Hz, 2H), 2.80 (t, J=12.1 Hz, 1H), 2.72 (d, J=11.0 Hz, 2H), 1.20 (t, J=7.5 Hz, 3H). LCMS calc. for C₃₄H₃₆ClF₃N₉O₅ [M+H]⁺: m/z=742.2; Found: 742.2.

### Example 289: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-5-(methylamino)benzamide

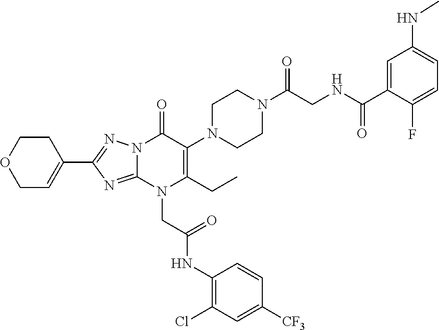 [SMILES: CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-fluoro-5-(methylamino)benzoic acid to afford the title product as a light white solid. LCMS calc. for C₃₅H₃₇ClF₄N₉O₅ [M+H]⁺: m/z=774.2; Found: 774.2.

### Example 290: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-5-hydroxybenzamide

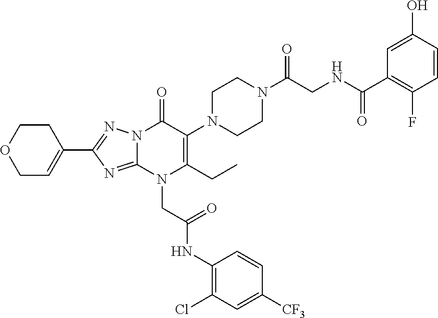 [SMILES: CCc1c(N2CCN(C(=O)CNC(=O)c3cc(O)ccc3F)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-fluoro-5-hydroxybenzoic acid to afford the title product as an off-white solid. LCMS calc. for C₃₄H₃₄ClF₄N₈O₆ [M+H]⁺: m/z=761.2 Found: 761.1.

### Example 291: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoro-5-hydroxybenzamide

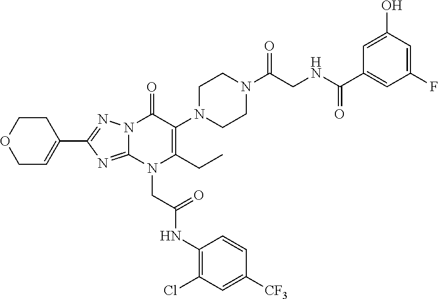 [SMILES: CCc1c(N2CCN(C(=O)CNC(=O)c3cc(O)cc(F)c3)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 3-fluoro-5-hydroxybenzoic acid to afford the title product as an off-white solid. LCMS calc. for C₃₄H₃₄ClF₄N₈O₆ [M+H]⁺: m/z=761.2; Found: 761.1.

### Example 292: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-fluoro-3-hydroxybenzamide

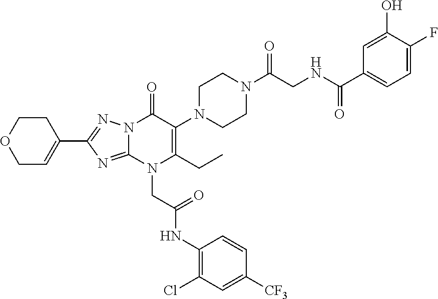 [SMILES: CCc1c(N2CCN(C(=O)CNC(=O)c3ccc(F)c(O)c3)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 4-fluoro-3-hydroxybenzoic acid to afford the title product as a white solid. LCMS calc. for C₃₄H₃₂ClF₄N₈O₆ [M−H]⁻: m/z=759.2; Found: 759.1.

### Example 293: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-3-hydroxybenzamide

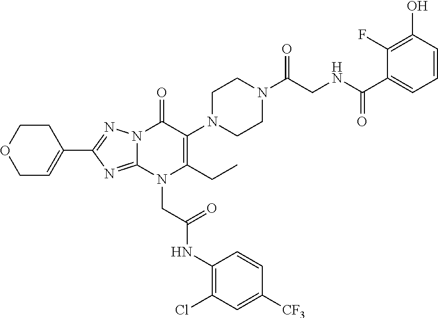 [SMILES: CCc1c(N2CCN(C(=O)CNC(=O)c3cccc(O)c3F)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-fluoro-3-hydroxybenzoic acid to afford the title product as a white solid. LCMS calc. for C₃₄H₃₂ClF₄N₈O₆ [M−H]⁻: m/z=759.2; Found: 759.1.

### Example 294: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-6-hydroxybenzamide

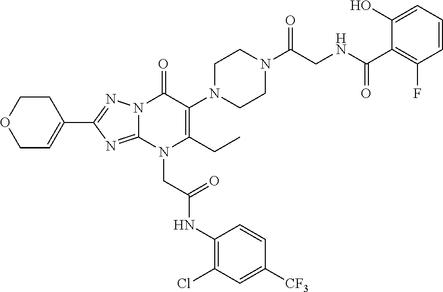 [SMILES: CCc1c(N2CCN(C(=O)CNC(=O)c3c(O)cccc3F)CC2)c(=O)n2nc(C3=CCOCC3)nc2n1CC(=O)Nc1ccc(C(F)(F)F)cc1Cl]

To a mixture of 2-fluoro-6-hydroxybenzoic acid (37.6 mg, 0.24 mmol), N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (150 mg, 0.24 mmol, Example 38) and EDCI (69 mg, 0.36 mmol) in DMF (1.5 mL) was added Et₃N (154.8 mg, 1.2 mmol) and HOBt (48.6 mg, 0.36 mmol). The mixture was stirred at r.t. overnight and purified by Prep-HPLC eluting with MeCN/H₂O (5-50%) to afford the title compound (23.1 mg, 12.5% yield) as a white solid. LCMS calc. for C₃₄H₃₄ClF₄N₈O₆ [M+H]⁺: m/z=761.2; Found: 761.0.

Example 295: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-hydroxy-6-methyl-2-oxo-2H-pyran-3-carboxamide

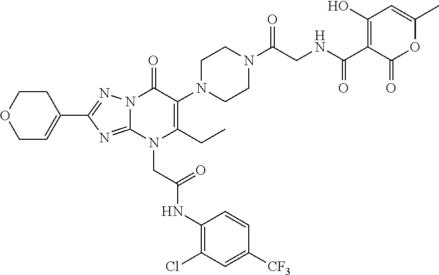 [SMILES: CCc1c(N2CCN(C(=O)CNC(=O)c3c(O)cc(C)oc3=O)CC2)c(=O)n2nc(C3=CCOCC3)nc2n1CC(=O)Nc1ccc(C(F)(F)F)cc1Cl]

### Step 1: 4-hydroxy-6-methyl-2-oxo-2H-pyran-3-carbonyl chloride

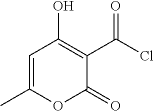

To solution of INT C2 (50 mg, 0.29 mmol) in DCM (5 mL) was added oxalyl chloride (44.2 mg, 0.35 mmol) and catalytic amount DMF (1 drop) under N₂ atmosphere at 0° C. (ice-water-bath). The resulting mixture was then stirred for 0.5 h. at r.t. The mixture was concentrated under reduced pressure to afford the title compound (54.5 mg. crude) as a yellow oil which was used directly in the next step.

### Step 2: N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-hydroxy-6-methyl-2-oxo-2H-pyran-3-carboxamid

To a solution of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (20 mg, 0.03 mmol, Example 38) and TEA (9.7 mg, 0.10 mmol) in DCM (2 mL) was added 4-hydroxy-6-methyl-2-oxo-2H-pyran-3-carbonyl chloride (6.6 mg, 0.035 mmol) at 0° C. under N₂ atmosphere. The resulting mixture was stirred at r.t. for 2 h., and concentrated. the residue was purified by Prep-HPLC eluting with MeCN/H₂O (45-60% with 0.1% NH₄HCO₃) to give the title compound (0.56 mg, 2.27% yield) as a white solid. LCMS calc. for C₃₄H₃₅ClF₃N₈O₈ [M+H]⁺: m/z=775.2; Found: 775.5.

### Example 296: N-(2-Chloro-4-(trifluoromethyl)phenyl)-2-(2-(3,6-dihydro-2H-pyran-4-yl)-5-ethyl-7-oxo-6-(4-(pyrazolo[1,5-c]pyrimidine-5-carbonyl)piperazin-1-yl)-[1,2,4]triazolo[1,5-a]pyrimidin-4(7H)-yl)acetamide

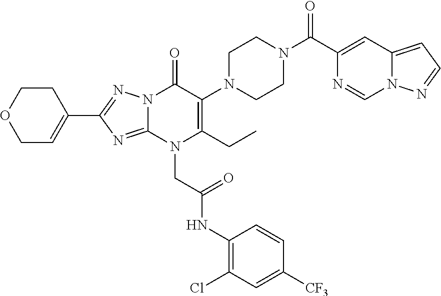 [SMILES: CCc1c(N2CCN(C(=O)c3cc4ccnn4cn3)CC2)c(=O)n2nc(C3=CCOCC3)nc2n1CC(=O)Nc1ccc(C(F)(F)F)cc1Cl]

This compound was prepared using procedures analogous to those described for Method C using INT B1 and pyrazolo[1,5-c]pyrimidine-5-carboxylic acid to afford the title product as a white solid. ¹H NMR (400 MHZ, DMSO-d₆) δ ppm 10.36 (s, 1H), 9.60 (t, J=1.3 Hz, 1H), 8.30 (d, J=2.2 Hz, 1H), 8.06 (dd, J=8.6, 6.0 Hz, 1H), 7.98 (dd, J=15.6, 1.8 Hz, 2H), 7.72 (dd, J=8.8, 2.1 Hz, 1H), 6.84 (ddd, J=7.7, 2.7, 1.3 Hz, 2H), 5.39-5.25 (m, 2H), 4.52 (d, J=12.7 Hz, 1H), 4.25 (q, J=2.9 Hz, 2H), 3.94 (d, J=12.8 Hz, 1H), 3.80 (t, J=5.4 Hz, 2H), 3.50 (td, J=11.6, 3.0 Hz, 2H), 3.00 (d, J=8.8 Hz, 3H), 2.87-2.75 (m, 1H), 2.64 (d, J=2.3 Hz, 1H), 2.52 (s, 4H), 1.19 (t, J=7.5 Hz, 3H). LCMS calc. for C₃₂H₃₁ClF₃N₁₀O₄ [M+H]⁺: m/z=711.2; Found: 711.0.

### Example 297:2-(6-(4-(2-Bromo-4-hydroxythieno[2,3-b]pyridine-5-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-fluoro-4-(trifluoromethyl)phenyl)acetamide

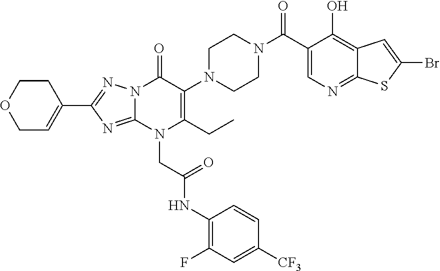 [SMILES: CCc1c(N2CCN(C(=O)c3cnc4sc(Br)cc4c3O)CC2)c(=O)n2nc(C3=CCOCC3)nc2n1CC(=O)Nc1ccc(C(F)(F)F)cc1F]

This compound was prepared using procedures analogous to those described for method A using INT B13 and INT C60 to afford the title product as a white solid. ¹H NMR (400 MHZ, DMSO-d₆) δ ppm 10.69 (s, 1H), 8.22 (t, J=8.0 Hz, 1H), 8.15 (d, J=12.8 Hz, 1H), 7.80 (d, J=9.4 Hz, 1H), 7.64 (s, 1H), 7.57 (d, J=9.4 Hz, 1H), 6.80 (s, 1H), 5.30 (s, 2H), 4.54-4.52 (m, 1H), 4.24 (d, J=2.6 Hz, 2H), 3.80 (t, J=5.4 Hz, 2H), 3.47 (t, J=10.8 Hz, 3H), 3.32-3.22 (m, 3H), 2.98-2.96 (m, 3H), 2.68-2.66 (m, 2H), 1.17 (t, J=7.4 Hz, 3H). LCMS calc. for C₃₃H₃₀BrF₄N₈O₅S [M+H]⁺: m/z=805.1; Found: 805.0.

Example 298:2-(2-(3,6-Dihydro-2H-pyran-4-yl)-5-ethyl-6-(4-(4-hydroxypyrrolo[1,2-b]pyridaz ine-3-carbonyl)piperazin-1-yl)-7-oxo-[1,2,4]triazolo[1,5-a]pyrimidin-4(7H)-yl)-N-(3,5-dimethylthiophen-2-yl)acetamide

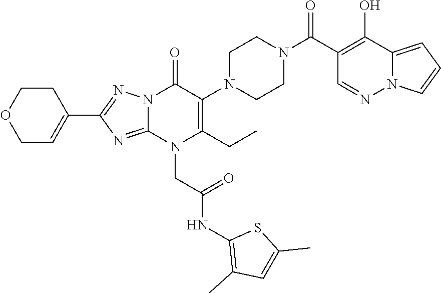 [SMILES: CCc1c(N2CCN(C(=O)c3cnn4cccc4c3O)CC2)c(=O)n2nc(C3=CCOCC3)nc2n1CC(=O)Nc1sc(C)cc1C]

This compound was prepared using procedures analogous to those described for method A using INT B29 and INT C31 to afford the title product as a light brown solid. ¹H NMR (400 MHZ, DMSO-d₆) δ ppm 11.91 (s, 1H), 10.46 (s, 1H), 7.96 (s, 1H), 7.81 (dd, J=2.8, 1.6 Hz, 1H), 6.93 (dd, J=4.4, 1.6 Hz, 1H), 6.81 (s, 1H), 6.76 (dd, J=4.4, 2.8 Hz, 1H), 6.43 (d, J=0.8 Hz, 1H), 5.20 (s, 2H), 4.26-4.24 (m, 2H), 4.09-4.07 (m, 1H), 3.81-3.79 (m, 2H), 3.52-3.50 (m, 2H), 3.15-3.13 (m, 2H), 2.97-2.95 (m, 2H), 2.75-2.73 (m, 2H), 2.52-2.50 (m, 3H), 2.28 (s, 3H), 2.08 (s, 3H), 1.17 (t, J=7.6 Hz, 3H). LCMS calc. for C₃₂H₃₆N₉O₅S [M+H]⁺: m/z=658.2; Found: 658.6.

### Example 299: N-(2-Chloro-4-(trifluoromethyl)phenyl)-2-(2-(3,6-dihydro-2H-pyran-4-yl)-5-ethyl-6-(4-(4-fluoro-1H-imidazole-5-carbonyl)piperazin-1-yl)-7-oxo-[1,2,4]triazolo[1,5-a]pyrimidin-4(7H)-yl)acetamide

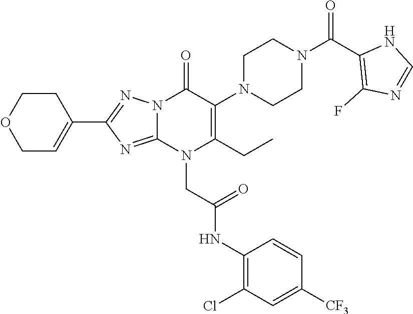 [SMILES: CCc1c(N2CCN(C(=O)c3[nH]cnc3F)CC2)c(=O)n2nc(C3=CCOCC3)nc2n1CC(=O)Nc1ccc(C(F)(F)F)cc1Cl]

This compound was prepared using procedures analogous to those described for method A using INT B1 and 4-fluoro-1H-imidazole-5-carboxylic acid to afford the title product as a white solid. ¹H NMR (400 MHZ, DMSO-d₆) δ ppm 10.36 (s, 1H), 8.06 (d, J=9.1 Hz, 2H), 7.97 (d, J=2.1 Hz, 1H), 7.72 (dd, J=8.7, 2.1 Hz, 1H), 6.85-6.81 (m, 1H), 5.32 (d, J=1.9 Hz, 2H), 4.25 (q, J=2.9 Hz, 2H), 4.20 (d, J=12.4 Hz, 1H), 3.80 (t, J=5.5 Hz, 2H), 3.70 (d, J=12.3 Hz, 1H), 3.42 (dd, J=13.9, 10.7 Hz, 2H), 3.21 (s, 2H), 2.99 (d, J=7.5 Hz, 2H), 2.84-2.66 (m, 4H), 1.21-1.17 (m, 3H). LCMS calc. for C₂₉H₂₉ClF₄N₉O₄ [M+H]⁺: m/z=678.2; Found: 678.2.

### Example 300: N-(2-Chloro-4-(trifluoromethyl)phenyl)-2-(2-(3,6-dihydro-2H-pyran-4-yl)-5-ethyl-6-(4-(3-fluoro-1H-pyrrolo[3,2-c]pyridine-2-carbonyl)piperazin-1-yl)-7-oxo-[1,2,4]triazolo[1,5-a]pyrimidin-4(7H)-yl)acetamide

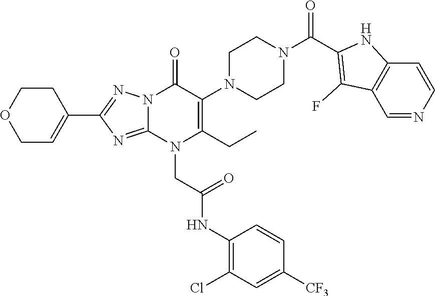 [SMILES: CCc1c(N2CCN(C(=O)c3[nH]c4ccncc4c3F)CC2)c(=O)n2nc(C3=CCOCC3)nc2n1CC(=O)Nc1ccc(C(F)(F)F)cc1Cl]

This compound was prepared using procedures analogous to those described for method A using INT B1 and INT C61 to afford the title product as a white solid. ¹H NMR (400 MHZ, DMSO-d₆) δ ppm 12.35 (s, 1H), 10.38 (s, 1H), 9.10 (s, 1H), 8.35 (d, J=6.1 Hz, 1H), 8.14-8.02 (m, 1H), 7.97 (d, J=2.1 Hz, 1H), 7.72 (dd, J=8.8, 2.1 Hz, 1H), 7.55-7.45 (m, 1H), 6.88-6.81 (m, 1H), 5.33 (s, 2H), 4.50 (s, 1H), 4.25 (q, J=2.9 Hz, 2H), 4.00 (s, 1H), 3.80 (t, J=5.5 Hz, 2H), 3.52 (d, J=3.4 Hz, 2H), 3.16 (d, J=11.5 Hz, 2H), 3.02 (d, J=7.8 Hz, 2H), 2.81 (d, J=7.9 Hz, 4H), 1.21 (t, J=7.4 Hz, 3H). LCMS calc. for C₃₃H₃₁ClF₄N₉O₄ [M+H]⁺: m/z=728.2; Found: 728.2.

### Example 301: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-methyloxazole-2-carboxamide

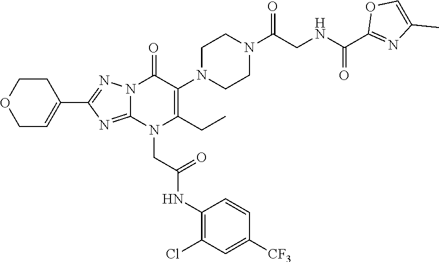 [SMILES: CCc1c(N2CCN(C(=O)CNC(=O)c3nc(C)co3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.08 mmol, Example 38) and 4-methyloxazole-2-carboxylic acid (10.1 mg, 0.08 mmol) in pyridine (0.5 mL) was added EDCI (23.1 mg, 0.12 mmol) at r.t. The mixture was stirred at r.t. for 3 h., and then concentrated under reduced pressure. The residue was purified by flash chromatography on a C18 column eluting with MeCN/H₂O (45-60%) to afford the title compound (2 mg) as a white solid. LCMS calc. for C₃₂H₃₂ClF₃N₉O₆ [M−H]⁻: m/z=730.2; Found: 730.1.

### Example 302: N-(2-Chloro-4-(trifluoromethyl)phenyl)-2-(2-(3,6-dihydro-2H-pyran-4-yl)-5-ethyl-6-(4-(4-methoxy-1H-pyrrolo[2,3-b]pyridine-2-carbonyl)piperazin-1-yl)-7-oxo-[1,2,4]triazolo[1,5-a]pyrimidin-4(7H)-yl)acetamide

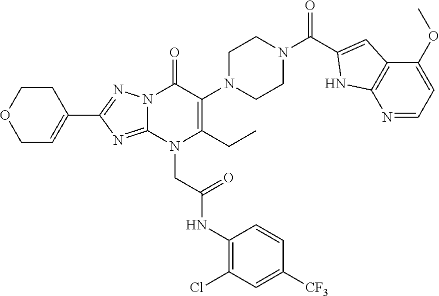 [SMILES: CCc1c(N2CCN(C(=O)c3cc4c(OC)ccnc4[nH]3)CC2)c(=O)n2nc(C3=CCOCC3)nc2n1CC(=O)Nc1ccc(C(F)(F)F)cc1Cl]

This compound was prepared using procedures analogous to those described for method A using INT B1 and 4-methoxy-1H-pyrrolo[2,3-b]pyridine-2-carboxylic acid to afford the title product as a white solid. ¹H NMR (400 MHZ, DMSO-d₆) δ ppm 12.12 (s, 1H), 10.37 (s, 1H), 8.21 (d, J=5.5 Hz, 1H), 8.06 (d, J=8.5 Hz, 1H), 7.97 (s, 1H), 7.72 (d, J=7.4 Hz, 1H), 6.83 (s, 1H), 6.73-6.69 (m, 2H), 5.33 (s, 2H), 4.42 (s, 2H), 4.25 (d, J=2.2 Hz, 2H), 3.96 (s, 3H), 3.80 (t, J=8.3 Hz, 2H), 3.49 (t, J=10.6 Hz, 6H), 3.02 (d, J=7.4 Hz, 2H), 2.78 (d, J=10.3 Hz, 2H), 1.24-1.19 (m, 3H). LCMS calc. for C₃₄H₃₄ClF₃N₉O₅ [M+H]⁺: m/z=740.2; Found: 740.3.

### Example 303: N-(2-Chloro-4-(trifluoromethyl)phenyl)-2-(2-(3,6-dihydro-2H-pyran-4-yl)-5-ethyl-6-(4-(4-hydroxy-5-methylpyrrolo[1,2-b]pyridazine-3-carbonyl)piperazin-1-yl)-7-oxo-[1,2,4]triazolo[1,5-a]pyrimidin-4(7H)-yl)acetamide

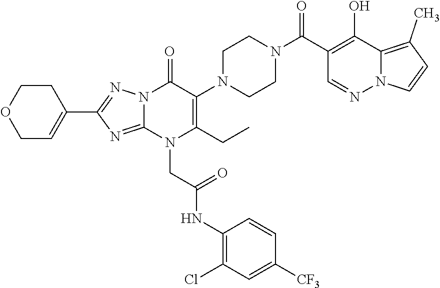 [SMILES: CCc1c(N2CCN(C(=O)c3cnn4ccc(C)c4c3O)CC2)c(=O)n2nc(C3=CCOCC3)nc2n1CC(=O)Nc1ccc(C(F)(F)F)cc1Cl]

This compound was prepared using procedures analogous to those described for method A using INT B1 and INT C62 to afford the title product as a white solid. ¹H NMR (400 MHZ, DMSO-d₆) δ ppm 12.50 (s, 1H), 10.38 (s, 1H), 8.06 (d, J=8.4 Hz, 1H), 7.97 (d, J=1.6 Hz, 1H), 7.72 (dd, J=8.4, 1.6 Hz, 2H), 7.39 (s, 1H), 6.84-6.82 (m, 1H), 6.33 (s, 1H), 5.32 (s, 2H), 4.26-4.24 (m, 2H), 4.17-4.15 (m, 2H), 3.81-3.79 (m, 2H), 3.49-3.47 (m, 2H), 3.33-3.31 (m, 2H), 3.17-3.15 (m, 2H), 3.02-2.99 (m, 2H), 2.72-2.70 (m, 2H), 2.48 (s, 3H), 1.21 (t, J=7.6 Hz, 3H). LCMS calc. for C₃₄H₃₄ClF₃N₉O₅ [M+H]⁺: m/z=740.2; Found: 740.4.

### Example 304: N-(2-Chloro-4-(trifluoromethyl)phenyl)-2-(2-(3,6-dihydro-2H-pyran-4-yl)-5-ethyl-6-(4-(2-hydroxypyrazolo[1,5-a]pyridine-3-carbonyl)piperazin-1-yl)-7-oxo-[1,2,4]triazolo[1,5-a]pyrimidin-4(7H)-yl) acetamide

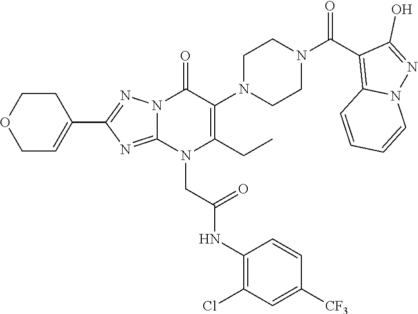 [SMILES: CCc1c(N2CCN(C(=O)c3c(O)nn4ccccc34)CC2)c(=O)n2nc(C3=CCOCC3)nc2n1CC(=O)Nc1ccc(C(F)(F)F)cc1Cl]

This compound was prepared using procedures analogous to those described for method A using INT B1 and 2-hydroxypyrazolo[1,5-a]pyridine-3-carboxylic acid to afford the title product as a white solid. ¹H NMR (400 MHZ, DMSO-d₆) δ ppm 10.43 (s, 1H), 8.47 (d, J=6.8 Hz, 1H), 8.06 (d, J=8.4 Hz, 1H), 7.97 (s, 1H), 7.72 (d, J=8.4 Hz, 1H), 7.60 (d, J=8.4 Hz, 1H), 7.32 (t, J=7.6 Hz, 1H), 6.91-6.79 (m, 2H), 5.33 (s, 2H), 4.27-4.25 (m, 2H), 4.19 (d, J=10.8 Hz, 2H), 3.81-3.79 (m, 2H), 3.52-3.50 (m, 4H), 3.16-3.09 (m, 2H), 3.02-3.00 (m, 2H), 2.74-2.72 (m, 2H), 1.21 (t, J=7.6 Hz, 3H). LCMS calc. for C₃₃H₃₂ClF₃N₉O₅ [M+H]⁺: m/z=726.2; Found: 726.2.

### Example 305: N-(2-Chloro-4-(trifluoromethyl)phenyl)-2-(2-(3,6-dihydro-2H-pyran-4-yl)-5-ethyl-6-(4-(6-hydroxythieno[3,2-b]pyridine-5-carbonyl)piperazin-1-yl)-7-oxo-[1,2,4]triazolo[1,5-a]pyrimidin-4(7H)-yl)acetamide

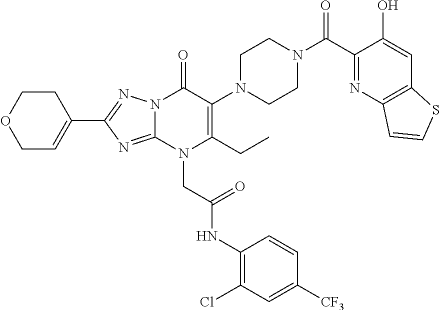 [SMILES: CCc1c(N2CCN(C(=O)c3nc4ccsc4cc3O)CC2)c(=O)n2nc(C3=CCOCC3)nc2n1CC(=O)Nc1ccc(C(F)(F)F)cc1Cl]

This compound was prepared using procedures analogous to those described for Method H using INT B1 and INT C63 to afford the title product as a white solid. ¹H NMR (400 MHZ, DMSO-d₆) δ ppm 10.37 (s, 1H), 8.05 (d, J=8.4 Hz, 1H), 7.96 (d, J=1.2 Hz, 1H), 7.87-7.86 (m, 2H), 7.71 (dd, J=8.4 Hz, 1.2 Hz, 1H), 7.44 (d, J=4.8 Hz, 1H), 6.85-6.81 (m, 1H), 5.31 (s, 2H), 4.57 (d, J=12.8 Hz, 1H), 4.27-4.23 (m, 2H), 3.80 (t, J=5.4 Hz, 2H), 3.55-3.43 (m, 2H), 3.34-3.31 (m, 2H), 3.26-3.20 (m, 2H), 3.00-2.96 (m, 3H), 2.82-2.80 (m, 1H), 2.60-2.58 (m, 1H), 1.18 (t, J=7.2 Hz, 3H). LCMS calc. for C₃₃H₃₁ClF₃N₈O₅S [M+H]⁺: m/z=743.2; Found: 743.1.

### Example 306: N-(2-Chloro-4-(trifluoromethyl)phenyl)-2-(2-(3,6-dihydro-2H-pyran-4-yl)-5-ethyl-6-(4-(3-hydroxyquinoline-2-carbonyl)piperazin-1-yl)-7-oxo-[1,2,4]triazolo[1,5-a]pyrimidin-4(7H)-yl)acetamide

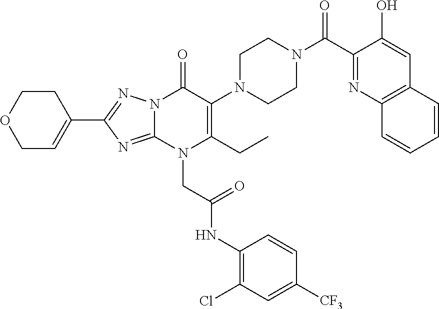 [SMILES: CCc1c(N2CCN(C(=O)c3nc4ccccc4cc3O)CC2)c(=O)n2nc(C3=CCOCC3)nc2n1CC(=O)Nc1ccc(C(F)(F)F)cc1Cl]

This compound was prepared using procedures analogous to those described for Method H using INT B1 and 3-hydroxyquinoline-2-carboxylic acid to afford the title product as a white solid. ¹H NMR (400 MHZ, DMSO-d₆) δ ppm 10.38 (s, 1H), 8.07 (dd, J=11.6, 8.4 Hz, 2H), 7.97 (d, J=1.6 Hz, 1H), 7.74-7.62 (m, 3H), 7.36 (t, J=8.8 Hz, 1H), 6.83 (s, 1H), 6.07 (s, 1H), 5.32 (s, 2H), 4.58-4.46 (m, 1H), 4.25 (d, J=2.4 Hz, 2H), 3.79 (dd, J=13.8, 8.4 Hz, 3H), 3.57-3.49 (m, 1H), 3.48-3.39 (m, 2H), 3.31-3.28 (m, 1H), 3.06 (d, J=10.8 Hz, 1H), 3.03-2.96 (m, 2H), 2.85 (d, J=9.2 Hz, 1H), 2.76-2.62 (m, 2H), 1.19 (t, J=7.6 Hz, 3H). LCMS calc. for C₃₅H₃₃ClF₃N₈O₅ [M+H]⁺: m/z=737.2; Found: 737.2.

### Example 307:2-(6-(4-(3-Aminoquinoline-2-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4 (trifluoromethyl)phenyl)acetamide

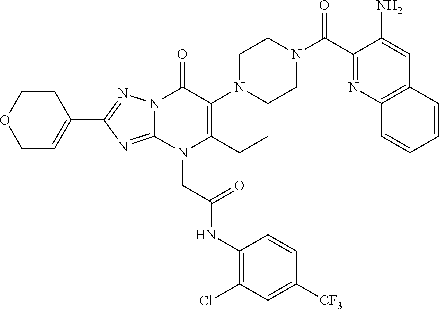 [SMILES: CCc1c(N2CCN(C(=O)c3nc4ccccc4cc3N)CC2)c(=O)n2nc(C3=CCOCC3)nc2n1CC(=O)Nc1ccc(C(F)(F)F)cc1Cl]

This compound was prepared using procedures analogous to those described for method J using INT B1 and 3-aminoquinoline-2-carboxylic acid to afford the title product as a yellow solid. ¹H NMR (400 MHZ, DMSO-d₆) δ ppm 10.37 (s, 1H), 8.06 (d, J=8.4 Hz, 1H), 7.97 (d, J=1.6 Hz, 1H), 7.81 (d, J=8.2 Hz, 1H), 7.71 (d, J=8.8 Hz, 1H), 7.67 (d, J=8.0 Hz, 1H), 7.46-7.37 (m, 3H), 6.83 (s, 1H), 5.53 (s, 2H), 5.32 (s, 2H), 4.61 (d, J=12.2 Hz, 1H), 4.25 (d, J=2.6 Hz, 2H), 3.80 (t, J=5.4 Hz, 2H), 3.52 (dt, J=17.6, 10.2 Hz, 3H), 3.32-3.30 (m, 3H), 3.09-2.96 (m, 3H), 2.85 (d, J=10.0 Hz, 1H), 2.63 (d, J=10.8 Hz, 1H), 1.19 (t, J=7.6 Hz, 3H). LCMS calc. for C₃₅H₃₄ClF₃N₉O₄ [M+H]⁺: m/z=736.2; Found: 736.3.

### Example 308:2-(6-(4-(2-Chloro-7-hydroxythiazolo[5,4-b]pyridine-6-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-fluoro-4-(trifluoromethyl)phenyl)acetamide

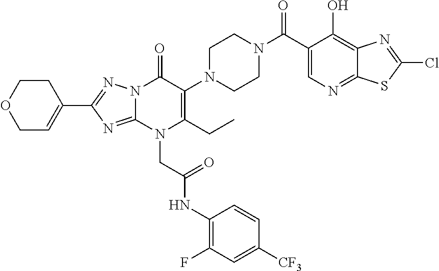 [SMILES: CCc1c(N2CCN(C(=O)c3cnc4sc(Cl)nc4c3O)CC2)c(=O)n2nc(C3=CCOCC3)nc2n1CC(=O)Nc1ccc(C(F)(F)F)cc1F]

A mixture of INT C64 (70 mg, 0.30 mmol), INT B13 (142 mg, 0.24 mmol), T₃P (547 mg, 1.52 mmol) and DIEA (196 mg, 1.52 mmol) in DCM (2 mL) was stirred at r.t. overnight. The reaction mixture was diluted with EtOAc (80 mL), washed with water, concentrated and purified by Prep-HPLC on a C18 column eluting with MeCN/H₂O (45-95%, with 0.1% NH₄HCO₃) to afford the title compound (17.1 mg, 9.6% yield) as a white solid. ¹H NMR (400 MHz, DMSO-d₆) δ ppm 10.71 (s, 1H), 8.23 (t, J=8.0 Hz, 1H), 8.09 (s, 1H), 7.83-7.75 (m, 1H), 7.56 (d, J=8.8 Hz, 1H), 7.14 (s, 1H), 6.80 (s, 1H), 5.30 (s, 2H), 4.56-4.52 (m, 1H), 4.24 (d, J=2.4 Hz, 2H), 3.80 (t, J=5.4 Hz, 2H), 3.48-3.38 (m, 5H), 2.97 (d, J=7.4 Hz, 3H), 2.68-2.62 (m, 3H), 1.17 (t, J=7.4 Hz, 3H). LCMS calc. for C₃₂H₂₉ClF₄N₉O₅S [M+H]⁺: m/z=762.2; Found: 762.2.

### Example 309:2-(2-(3,6-Dihydro-2H-pyran-4-yl)-5-ethyl-6-(4-(5-(2-fluorophenyl)-4H-1,2,4-triazole-3-carbonyl)piperazin-1-yl)-7-oxo-[1,2,4]triazolo[1,5-a]pyrimidin-4(7H)-yl)-N-(2-fluoro-4-(trifluoromethyl)phenyl)acetamide

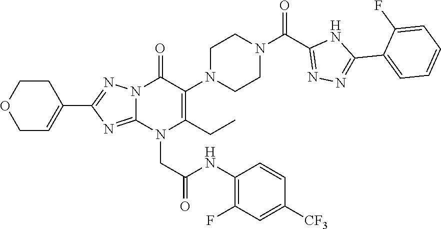 [SMILES: CCc1c(N2CCN(C(=O)c3nnc(-c4ccccc4F)[nH]3)CC2)c(=O)n2nc(C3=CCOCC3)nc2n1CC(=O)Nc1ccc(C(F)(F)F)cc1F]

To a mixture of 5-(2-fluorophenyl)-4H-1,2,4-triazole-3-carboxylic acid (11.3 mg, 0.05 mmol), INT B13 (30 mg, 0.05 mmol) and EDCI (15.5 mg, 0.08 mmol) in DMF (0.5 mL) was added Et₃N (27.3 mg, 0.27 mmol) and HOBt (11 mg, 0.08 mmol). The mixture was stirred at r.t. overnight. The reaction mixture was purified by Prep-HPLC eluting with MeCN/H₂O (5-60%) to afford the title compound (7.1 mg, 17% yield) as a white solid. LCMS calc. for C₃₄H₃₀F₅N₁₀O₄ [M−H]⁻: m/z=737.1; Found: 737.1.

### Example 310:2-(6-(4-(5-(Difluoromethyl)-4H-1,2,4-triazole-3-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-fluoro-4-(trifluoromethyl)phenyl)acetamide

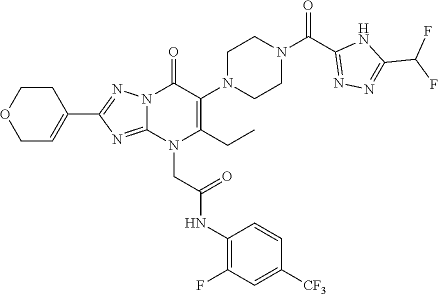 [SMILES: CCc1c(N2CCN(C(=O)c3nnc(C(F)F)[nH]3)CC2)c(=O)n2nc(C3=CCOCC3)nc2n1CC(=O)Nc1ccc(C(F)(F)F)cc1F]

To a mixture of INT B13 (50.0 mg, 0.09 mmol), 3-(difluoromethyl)-1H-1,2,4-triazole-5-carboxylic acid (22 mg, 0.14 mmol) and TCFH (51 mg, 0.18 mmol) in DMF (1 mL) was added NMI (38.0 mg, 0.45 mmol). The mixture was stirred at r.t. for 2 h. The reaction mixture was concentrated under reduced pressure and the residue purified by flash chromatography on a C18 column eluting with MeCN/H₂O (45% with 0.05% NH₄HCO₃) to afford the title compound (35.6 mg, 59.7% yield) as a white solid. LCMS calc. for C₂₉H₂₉F₆N₁₀O₄ [M+H]⁺: m/z=695.2; Found: 695.2. Example 311:2-(2-(3,6-Dihydro-2H-pyran-4-yl)-5-ethyl-7-oxo-6-(4-(5-(trifluoromethyl)-4H-1,2,4-triazole-3-carbonyl)piperazin-1-yl)-[1,2,4]triazolo[1,5-a]pyrimidin-4(7H)-yl)-N-(2-fluoro-4-(trifluoromethyl)phenyl)acetamide

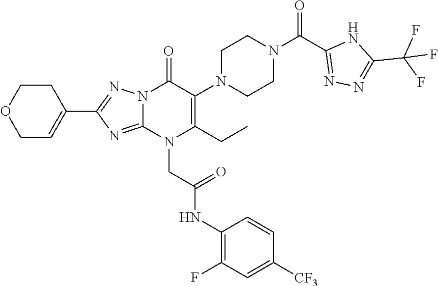 [SMILES: CCc1c(N2CCN(C(=O)c3nnc(C(F)(F)F)[nH]3)CC2)c(=O)n2nc(C3=CCOCC3)nc2n1CC(=O)Nc1ccc(C(F)(F)F)cc1F]

To a mixture of 3-(trifluoromethyl)-1H-1,2,4-triazole-5-carboxylic acid (9.9 mg, 0.05 mmol), INT B13 (30 mg, 0.05 mmol) in DMF (0.5 mL) was added NMI (22.14 mg, 0.27 mmol) and TCFH (18.1 mg, 0.07 mmol). The mixture was stirred at r.t. overnight. The reaction mixture was purified by Prep-HPLC eluting with MeCN/H₂O (5-65%) to afford the title compound (8.7 mg, 22% yield) as a white solid. LCMS calc. for C₂₉H₂₆F₇N₁₀O₄ [M−H]⁻: m/z=711.2; Found: 711.1.

### Example 312:2-(2-(3,6-Dihydro-2H-pyran-4-yl)-5-ethyl-6-(4-(5-methyl-4H-1,2,4-triazole-3-carbonyl)piperazin-1-yl)-7-oxo-[1,2,4]triazolo[1,5-a]pyrimidin-4(7H)-yl)-N-(2-fluoro-4-(trifluoromethyl)phenyl)acetamide

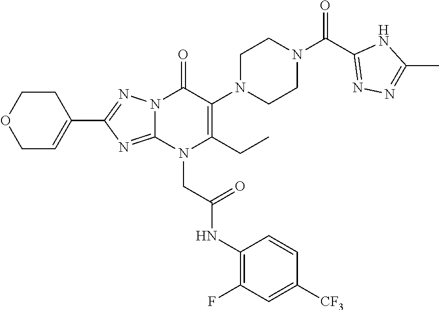 [SMILES: CCc1c(N2CCN(C(=O)c3nnc(C)[nH]3)CC2)c(=O)n2nc(C3=CCOCC3)nc2n1CC(=O)Nc1ccc(C(F)(F)F)cc1F]

To a mixture of 5-methyl-4H-1,2,4-triazole-3-carboxylic acid (7 mg, 0.05 mmol), INT B13 (30 mg, 0.05 mmol) and EDCI (15.7 mg, 0.08 mmol) in DMF (0.5 mL) was added Et₃N (27.5 mg, 0.27 mmol) and HOBt (11 mg, 0.08 mmol). The mixture was stirred at r.t. overnight. The reaction mixture was purified by Prep-HPLC eluting with MeCN/H₂O (5-65%) to afford the title compound (7.2 mg, 20% yield) as a white solid. LCMS calc. for C₂₉H₃₁F₄N₁₀O₄ [M+H]⁺: m/z=659.2; Found: 659.1. Example 313:2-(6-(4-(1H-Pyrrolo[3,2-c]pyridine-2-carbonyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

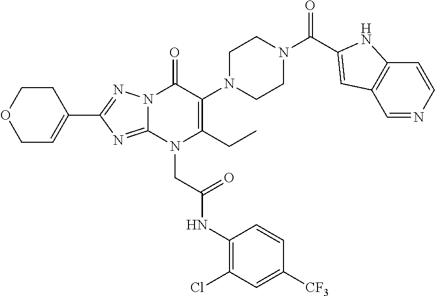 [SMILES: CCc1c(N2CCN(C(=O)c3cc4cnccc4[nH]3)CC2)c(=O)n2nc(C3=CCOCC3)nc2n1CC(=O)Nc1ccc(C(F)(F)F)cc1Cl]

This compound was prepared using procedures analogous to those described for Method C using INT B1 and 1H-pyrrolo[3,2-c]pyridine-2-carboxylic acid to afford the title product as a white solid. ¹H NMR (400 MHz, DMSO-d₆) δ ppm 12.15 (s, 1H), 10.38 (s, 1H), 8.94 (d, J=4.5 Hz, 1H), 8.24 (dd, J=5.9, 2.4 Hz, 1H), 8.06 (d, J=8.5 Hz, 1H), 7.97 (d, J=2.1 Hz, 1H), 7.72 (dd, J=8.7, 2.2 Hz, 1H), 7.41 (d, J=6.1 Hz, 1H), 7.01 (s, 1H), 6.83 (p, J=1.5 Hz, 1H), 5.33 (s, 2H), 4.47 (s, 2H), 4.25 (q, J=2.8 Hz, 2H), 3.80 (t, J=5.4 Hz, 2H), 3.53 (dd, J=11.6, 2.8 Hz, 4H), 3.02 (d, J=7.5 Hz, 2H), 2.80 (d, J=10.9 Hz, 2H), 2.67 (t, J=1.9 Hz, 1H), 2.33 (q, J=1.9 Hz, 1H), 1.21 (t, J=7.4 Hz, 3H). LCMS calc. for C₃₃H₃₂ClF₃N₉O₄ [M+H]⁺: m/z=710.2; Found: 710.0.

### Example 314: N-(2-Chloro-4-(trifluoromethyl)phenyl)-2-(2-(3,6-dihydro-2H-pyran-4-yl)-5-ethyl-6-(4-(3-hydroxy-5-methoxypyridazine-4-carbonyl)piperazin-1-yl)-7-oxo-[1,2,4]triazolo[1,5-a]pyrimidin-4(7H)-yl)acetamide

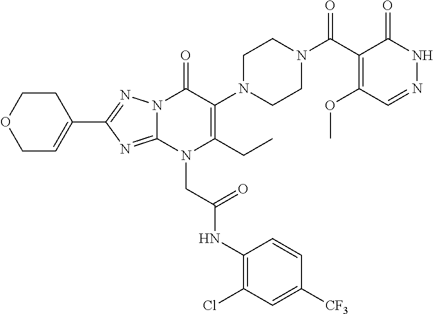 [SMILES: CCc1c(N2CCN(C(=O)c3c(OC)cn[nH]c3=O)CC2)c(=O)n2nc(C3=CCOCC3)nc2n1CC(=O)Nc1ccc(C(F)(F)F)cc1Cl]

This compound was prepared using procedures analogous to those described for Method C using INT B1 and INT C65 to afford the title product as a white solid. ¹HNMR (400 MHZ, DMSO-d₆) δ ppm 12.52 (s, 1H), 10.36 (s, 1H), 8.09-8.01 (m, 2H), 7.97 (d, J=1.6 Hz, 1H), 7.75-7.70 (m, 1H), 6.83 (s, 1H), 5.32 (s, 2H), 4.25 (d, J=2.4 Hz, 2H), 3.80 (t, J=5.4 Hz, 2H), 3.76 (d, J=2.4 Hz, 3H), 3.68 (d, J=12.0 Hz, 2H), 3.56 (t, J=10.6 Hz, 2H), 3.18 (t, J=11.2 Hz, 2H), 2.98 (d, J=7.4 Hz, 2H), 2.74 (d, J=11.2 Hz, 2H), 2.69-2.63 (m, 1H), 2.36-2.30 (m, 1H), 1.19 (t, J=7.5 Hz, 3H). LCMS calc. for C₃₁H₃₂ClF₃N₉O₆ [M+H]⁺: m/z=718.2; Found: 718.2.

Example 315: N-(2-Chloro-4-(trifluoromethyl)phenyl)-2-(2-(3,6-dihydro-2H-pyran-4-yl)-5-ethyl-6-(4-(6-fluoro-1H-indole-7-carbonyl)piperazin-1-yl)-7-oxo-[1,2,4]triazolo[1,5-a]pyrimidin-4(7H)-yl)acetamide

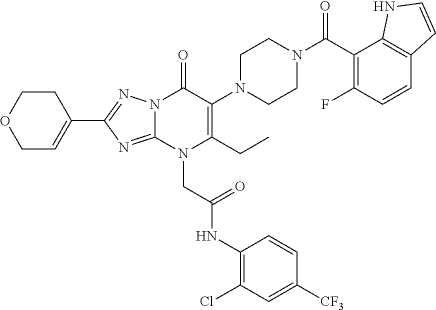 [SMILES: CCc1c(N2CCN(C(=O)c3c(F)ccc4cc[nH]c34)CC2)c(=O)n2nc(C3=CCOCC3)nc2n1CC(=O)Nc1ccc(C(F)(F)F)cc1Cl]

This compound was prepared using procedures analogous to those described for Method C using INT B1 and 6-fluoro-1H-indole-7-carboxylic acid to afford the title product as a white solid. ¹H NMR (400 MHZ, DMSO-d₆) δ ppm 11.31 (s, 1H), 10.34 (s, 1H), 8.08-8.02 (m, 1H), 7.99-7.93 (m, 1H), 7.71 (dd, J=9.2, 2.1 Hz, 1H), 7.61 (dd, J=8.7, 5.2 Hz, 1H), 7.36 (t, J=2.8 Hz, 1H), 6.94 (t, J=9.4 Hz, 1H), 6.83 (p, J=1.5 Hz, 1H), 6.50 (dd, J=3.1, 1.9 Hz, 1H), 5.31 (s, 2H), 4.66 (d, J=12.6 Hz, 1H), 4.25 (d, J=2.9 Hz, 2H), 3.80 (t, J=5.4 Hz, 2H), 3.51 (s, 4H), 3.30 (s, 2H), 3.12-3.04 (m, 1H), 2.99 (d, J=8.0 Hz, 2H), 2.85 (d, J=11.3 Hz, 1H), 2.64 (d, J=10.6 Hz, 1H), 1.19 (t, J=7.4 Hz, 3H). LCMS calc. for C₃₄H₃₂ClF₄N₈O₄ [M+H]⁺: m/z=727.2; Found: 727.2.

### Example 316: N-(2-Chloro-4-(trifluoromethyl)phenyl)-2-(2-(3,6-dihydro-2H-pyran-4-yl)-5-ethyl-7-oxo-6-(4-(2-oxo-2,3-dihydro-1H-imidazole-4-carbonyl)piperazin-1-yl)-[1,2,4]triazolo[1,5-a]pyrimidin-4(7H)-yl)acetamide

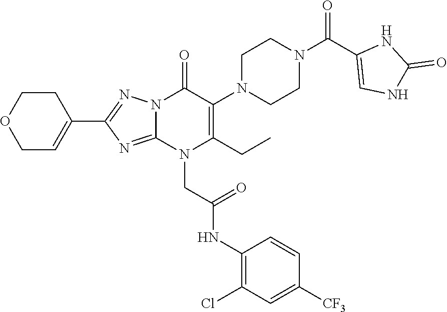 [SMILES: CCc1c(N2CCN(C(=O)c3c[nH]c(=O)[nH]3)CC2)c(=O)n2nc(C3=CCOCC3)nc2n1CC(=O)Nc1ccc(C(F)(F)F)cc1Cl]

This compound was prepared using procedures analogous to those described for Method C using INT B1 and 2-oxo-2,3-dihydro-1H-imidazole-4-carboxylic acid to afford the title product as a white solid. ¹H NMR (400 MHZ, DMSO-d₆) δ ppm 10.22 (s, 1H), 8.14-8.04 (m, 1H), 7.97 (d, J=2.2 Hz, 1H), 7.88 (s, 1H), 7.72 (dd, J=8.6, 2.1 Hz, 1H), 7.12 (s, 1H), 6.93 (t, J=2.3 Hz, 1H), 6.83 (s, 1H), 5.32 (s, 2H), 4.34-4.21 (m, 4H), 3.80 (t, J=5.6 Hz, 2H), 3.47-3.40 (m, 4H), 3.12 (d, J=12.2 Hz, 2H), 2.99 (d, J=7.5 Hz, 2H), 2.72 (d, J=11.1 Hz, 2H), 1.19 (t, J=7.6 Hz, 3H). LCMS calc. for C₂₉H₃₀ClF₃N₉O₅ [M+H]⁺: m/z=676.2; Found: 676.2.

### Example 317: N-(2-Chloro-4-(trifluoromethyl)phenyl)-2-(2-(3,6-dihydro-2H-pyran-4-yl)-5-ethyl-7-oxo-6-(4-(2-oxo-5-(trifluoromethyl)-2,3-dihydrooxazole-4-carbonyl)piperazin-1-yl)-[1,2,4]triazolo[1,5-a]pyrimidin-4(7H)-yl)acetamide

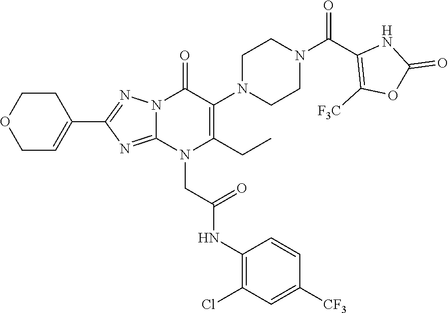 [SMILES: CCc1c(N2CCN(C(=O)c3[nH]c(=O)oc3C(F)(F)F)CC2)c(=O)n2nc(C3=CCOCC3)nc2n1CC(=O)Nc1ccc(C(F)(F)F)cc1Cl]

This compound was prepared using procedures analogous to those described for method I using INT B1 and INT C66 to afford the title product as a white solid. ¹H NMR (400 MHZ, DMSO-d₆) ppm 12.30 (s, 1H), 10.36 (s, 1H), 8.06 (d, J=8.6 Hz, 1H), 7.97 (s, 1H), 7.72 (d, J=6.8 Hz, 1H), 6.83 (s, 1H), 5.32 (s, 2H), 4.42 (d, J=12.8 Hz, 1H), 4.25 (s, 2H), 3.90 (d, J=10.8 Hz, 1H), 3.80 (t, J=5.5 Hz, 2H), 3.43 (d, J=12.3 Hz, 4H), 2.98 (s, 4H), 2.78 (s, 2H), 1.19 (t, J=7.4 Hz, 3H). LCMS calc. for C₃₀H₂₈ClF₆N₈O₆ [M+H]⁺: m/z=745.2; Found: 745.2.

### Example 318:2-(2-(3,6-Dihydro-2H-pyran-4-yl)-5-ethyl-6-(4-(4-hydroxypyrrolo[1,2-b]pyridazine-3-carbonyl)piperazin-1-yl)-7-oxo-[1,2,4]triazolo[1,5-a]pyrimidin-4(7H)-yl)-N-(2-fluoro-4-(trifluoromethyl)phenyl)acetamide

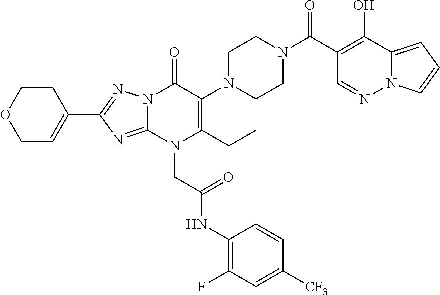 [SMILES: CCc1c(N2CCN(C(=O)c3cnn4cccc4c3O)CC2)c(=O)n2nc(C3=CCOCC3)nc2n1CC(=O)Nc1ccc(C(F)(F)F)cc1F]

To a mixture of INT C31 (9.7 mg, 0.05 mmol), INT B13 (30 mg, 0.05 mmol) in DMF (0.5 mL) was added NMI (22.14 mg, 0.27 mmol) and TCFH (18.2 mg, 0.07 mmol). The mixture was stirred at r.t. overnight. The reaction mixture was purified by Prep-HPLC eluting with MeCN/H₂O (5-50%) to afford the title compound (5.2 mg, 13% yield) as a white solid. LCMS calc. for C₃₃H₃₀F₄N₉O₅ [M−H]⁻: m/z=708.2; Found: 708.1.

Example 319:2-Chloro-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-6-fluorobenzamide

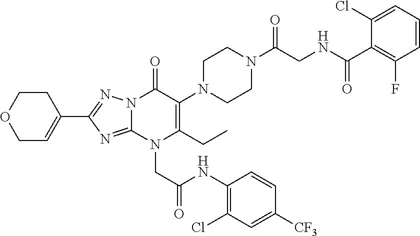 [SMILES: CCc1c(N2CCN(C(=O)CNC(=O)c3c(F)cccc3Cl)CC2)c(=O)n2nc(C3=CCOCC3)nc2n1CC(=O)Nc1ccc(C(F)(F)F)cc1Cl]

This compound was prepared using procedures analogous to those described for method I using INT B1 and (2-chloro-6-fluorobenzoyl)glycine to afford the title product as a white solid. LCMS calc. for C₃₄H₃₁C₁₂F₄N₈O₅ [M−H]⁻: m/z=777.2; Found: 777.1.

### Example 320: N-(2-(4-(4-(2-((2-Chloro-4-(Trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-methylbutanamide

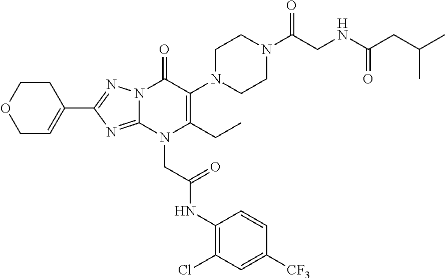 [SMILES: CCc1c(N2CCN(C(=O)CNC(=O)CC(C)C)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 3-methylbutanoic acid to afford the title product as an off-white solid. LCMS calc. for C₃₂H₃₉ClF₃N₈O₅ [M+H]⁺: m/z=707.3; Found: 707.1.

### Example 321: (R)—N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)tetrahydrofuran-2-carboxamide

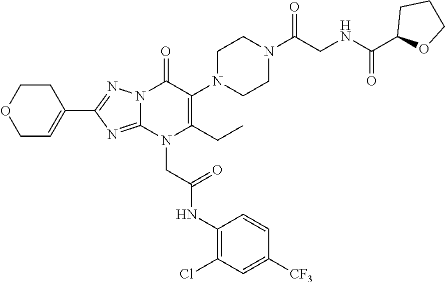 [SMILES: CCc1c(N2CCN(C(=O)CNC(=O)[C@H]3CCCO3)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and (R)-tetrahydrofuran-2-carboxylic acid to afford the title product as an off-white solid. LCMS calc. for C₃₂H₃₇ClF₃N₈O₆ [M+H]⁺: m/z=721.2; Found: 721.1.

### Example 322: (S)—N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)tetrahydrofuran-2-carboxamide

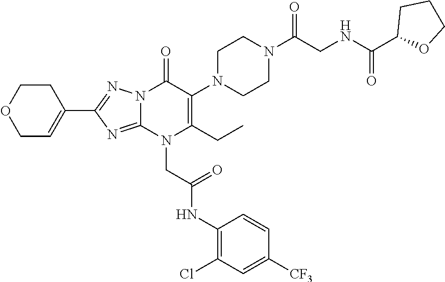 [SMILES: CCc1c(N2CCN(C(=O)CNC(=O)[C@@H]3CCCO3)CC2)c(=O)n2nc(C3=CCOCC3)nc2n1CC(=O)Nc1ccc(C(F)(F)F)cc1Cl]

This compound was prepared using procedures analogous to those described for method I using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and (S)-tetrahydrofuran-2-carboxylic acid to afford the title product as an off-white solid. LCMS calc. for C₃₂H₃₇ClF₃N₈O₆ [M+H]⁺: m/z=721.2; Found: 721.1.

### Example 323:2-(6-(4-(N-Acetyl-N-methylglycyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

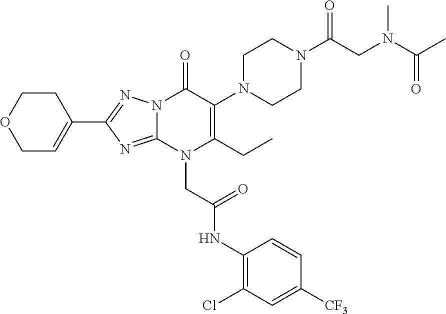 [SMILES: CCc1c(N2CCN(C(=O)CN(C)C(C)=O)CC2)c(=O)n2nc(C3=CCOCC3)nc2n1CC(=O)Nc1ccc(C(F)(F)F)cc1Cl]

This compound was prepared using procedures analogous to those described for method A using INT B1 and N-acetyl-N-methylglycine to afford the title product as a white solid. ¹H NMR (400 MHZ, DMSO-d₆) δ ppm 10.35 (s, 1H), 8.06 (d, J=8.5 Hz, 1H), 7.97 (s, 1H), 7.77-7.66 (m, 1H), 6.83 (s, 1H), 5.32 (s, 2H), 4.49-4.28 (m, 2H), 4.25 (d, J=2.7 Hz, 3H), 4.16 (t, J=13.9 Hz, 1H), 3.80 (t, J=5.4 Hz, 3H), 3.50 (dd, J=24.1, 12.2 Hz, 1H), 3.38 (d, J=14.4 Hz, 1H), 3.20 (dd, J=24.5, 12.6 Hz, 1H), 3.04-2.94 (m, 4H), 2.84-2.76 (m, 2H), 2.76-2.65 (m, 3H), 1.95 (d, J=62.0 Hz, 3H), 1.19 (t, J=7.4 Hz, 3H). LCMS calc. for C₃₀H₃₅ClF₃N₈O₅ [M+H]⁺: m/z=679.2; Found: 679.3.

### Example 324:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(3-methylisoxazol-5-yl)-2-oxoacetamide

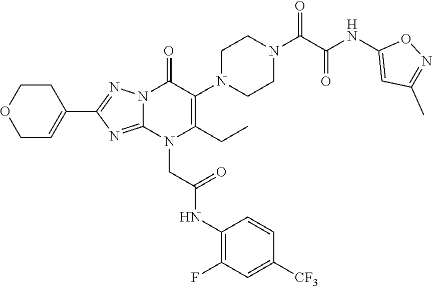 [SMILES: CCc1c(N2CCN(C(=O)C(=O)Nc3cc(C)no3)CC2)c(=O)n2nc(C3=CCOCC3)nc2n1CC(=O)Nc1ccc(C(F)(F)F)cc1F]

To a mixture of INT B13 (50 mg, 0.09 mmol) in pyridine (1 mL) was added 2-((3-methylisoxazol-5-yl)amino)-2-oxoacetic acid (INT C70, 23 mg, 0.14 mmol), EDCI (26 mg, 0.14 mmol). The mixture was stirred at r.t. for 2 h., and then concentrated under reduced pressure. The residue was purified by Prep-HPLC on a C18 column eluting with MeCN/H₂O (50%) to afford the title compound (13.4 mg, 21% yield) as a white solid. LCMS calc. for C₃₁H₃₀F₄N₉O₆ [M−H]⁻: m/z=700.2; Found: 700.2.

### Example 325:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(3-methylisothiazol-5-yl)-2-oxoacetamide

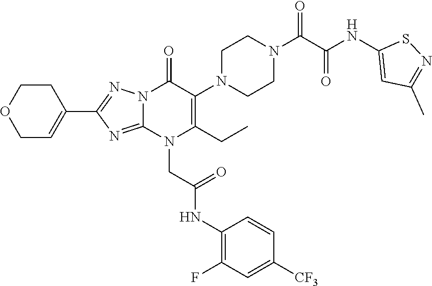 [SMILES: CCc1c(N2CCN(C(=O)C(=O)Nc3cc(C)ns3)CC2)c(=O)n2nc(C3=CCOCC3)nc2n1CC(=O)Nc1ccc(C(F)(F)F)cc1F]

To a mixture of INT B13 (60 mg, 0.11 mmol) in DMF (1 mL) was added 2-((3-methylisothiazol-5-yl)amino)-2-oxoacetic acid (30 mg, 0.16 mmol), NMI (27 mg, 0.33 mmol), TCFH (46 mg, 0.16 mmol). The mixture was stirred at r.t. for 2 h., and then concentrated under reduced pressure. The residue was purified by Prep-HPLC on a C18 column eluting with MeCN/H₂O (50%) to afford the title compound (19.2 mg, 25% yield) as a white solid. LCMS calc. for C₃₁H₃₂F₄N₉O₅S [M+H]⁺: m/z=718.2; Found: 718.1.

### Example 326:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N,N-dimethyl-2-oxoacetamide

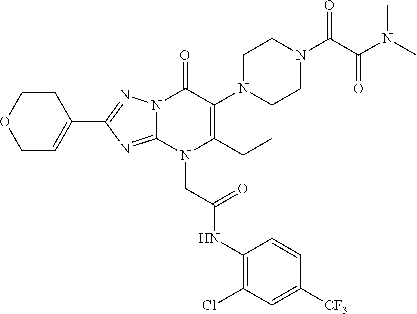 [SMILES: CCc1c(N2CCN(C(=O)C(=O)N(C)C)CC2)c(=O)n2nc(C3=CCOCC3)nc2n1CC(=O)Nc1ccc(C(F)(F)F)cc1Cl]

Method K: To a solution of INT B1 (25 mg, 0.04 mmol), 2-(dimethylamino)-2-oxoacetic acid (5 mg, 0.04 mmol) and HATU (22 mg) in MeCN (1.5 mL) was added DIEA (16 mg, 0.12 mmol). The reaction was stirred at r.t. under N₂ overnight. The mixture was concentrated under reduced pressure and purified by Prep-HPLC on a C18 column eluting with MeCN/H₂O (45-51%, with 0.1% FA) to afford the title compound (9.7 mg, 34% yield) as a white solid. ¹H NMR (400 MHZ, DMSO-d₆) δ ppm 10.36 (s, 1H), 8.06 (d, J=8.4 Hz, 1H), 7.97 (d, J=2.1 Hz, 1H), 7.72 (dd, J=8.8, 2.2 Hz, 1H), 6.84-6.58 (m, 1H), 5.25 (d, J=51.3 Hz, 2H), 4.40-4.19 (m, 3H), 3.81 (dt, J=7.1, 5.4 Hz, 2H), 3.45 (t, J=11.2 Hz, 2H), 3.30 (s, 6H), 2.99 (d, J=7.0 Hz, 1H), 2.95 (d, J=1.9 Hz, 3H), 2.89 (d, J=2.3 Hz, 3H), 2.82-2.69 (m, 2H), 1.24-1.12 (m, 3H). LCMS calc. for C₂₉H₃₃ClF₃N₈O₅ [M+H]⁺: m/z=665.2; Found: 665.2.

Example 327:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-phenylacetamide

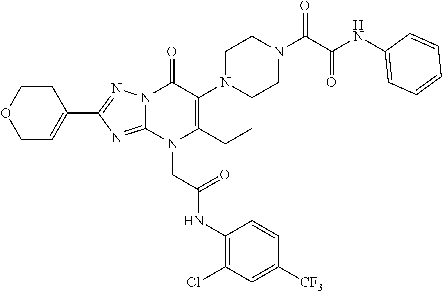 [SMILES: CCc1c(N2CCN(C(=O)C(=O)Nc3ccccc3)CC2)c(=O)n2nc(C3=CCOCC3)nc2n1CC(=O)Nc1ccc(C(F)(F)F)cc1Cl]

This compound was prepared using procedures analogous to those described for Method K using INT B1 and 2-oxo-2-(phenylamino)acetic acid to afford the title product as a white solid. ¹H NMR (400 MHZ, DMSO-d₆) δ ppm 10.84 (s, 1H), 10.36 (s, 1H), 8.07 (dd, J=8.6, 6.0 Hz, 1H), 7.96 (d, J=2.1 Hz, 1H), 7.72 (dd, J=8.8, 2.2 Hz, 1H), 7.67 (dq, J=8.8, 1.8 Hz, 2H), 7.41-7.30 (m, 2H), 7.16-7.09 (m, 1H), 6.89-6.76 (m, 1H), 5.40-5.25 (m, 2H), 4.37 (d, J=12.3 Hz, 1H), 4.25 (q, J=2.9 Hz, 2H), 3.87-3.76 (m, 3H), 3.55-3.44 (m, 2H), 3.05-2.90 (m, 3H), 2.78 (dd, J=24.0, 11.0 Hz, 3H), 2.52-2.50 (m, 2H), 1.19 (t, J=7.4 Hz, 3H). LCMS calc. for C₃₃H₃₃ClF₃N₈O₅ [M+H]⁺: m/z=713.2; Found: 713.2.

### Example 328: (E)-2-(2-(3,6-Dihydro-2H-pyran-4-yl)-5-ethyl-6-(4-(3-(4-hydroxypyridin-3-yl) acryloyl)piperazin-1-yl)-7-oxo-[1,2,4]triazolo[1,5-a]pyrimidin-4(7H)-yl)-N-(2-fluoro-4-(trifluoromethyl)phenyl)acetamide

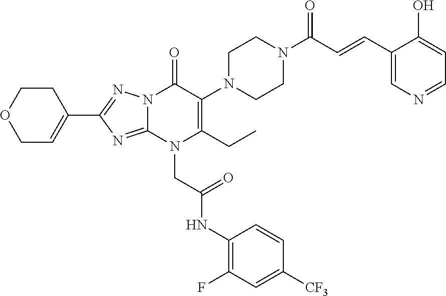 [SMILES: CCc1c(N2CCN(C(=O)/C=C/c3cnccc3O)CC2)c(=O)n2nc(C3=CCOCC3)nc2n1CC(=O)Nc1ccc(C(F)(F)F)cc1F]

A mixture of (E)-3-(4-hydroxypyridin-3-yl) acrylic acid (17 mg, 0.1 mmol), INT B13 (33 mg, 0.06 mmol), EDCI (27 mg, 0.15 mmol), HOBt (20 mg, 0.15 mmol) and TEA (50 mg, 0.5 mmol) in DMF (1 mL) was stirred at r.t. overnight. The mixture was concentrated under reduced pressure. The residue was purified by flash chromatography on a C18 column eluting with MeCN/H₂O (40-55% with 0.1% NH₄HCO₃) to afford the title compound (18.3 mg) as a white solid. LCMS calc. for C₃₃H₃₁F₄N₈O₅ [M−H]⁻: m/z=695.2; Found: 695.1.

### Example 329:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-methyl-2-oxoacetamide

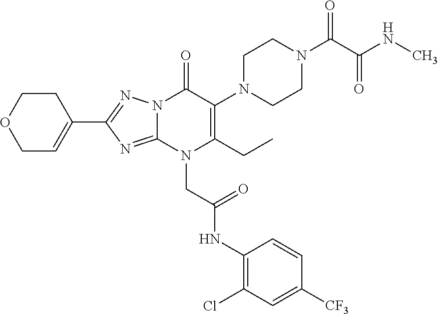 [SMILES: CCc1c(N2CCN(C(=O)C(=O)NC)CC2)c(=O)n2nc(C3=CCOCC3)nc2n1CC(=O)Nc1ccc(C(F)(F)F)cc1Cl]

This compound was prepared using procedures analogous to those described for Method K using INT B1 and 2-(methylamino)-2-oxoacetic acid to afford the title product as a white solid. ¹H NMR (400 MHZ, DMSO-d₆) δ ppm 10.37 (s, 1H), 8.68 (d, J=4.8 Hz, 1H), 8.06 (d, J=8.5 Hz, 1H), 7.96 (d, J=2.1 Hz, 1H), 7.74-7.69 (m, 1H), 6.85-6.80 (m, 1H), 5.32 (s, 2H), 4.32 (d, J=12.5 Hz, 1H), 4.25 (q, J=2.8 Hz, 2H), 3.88 (d, J=12.5 Hz, 1H), 3.80 (t, J=5.4 Hz, 2H), 3.48-3.37 (m, 2H), 3.28-3.21 (m, 2H), 2.99 (q, J=6.3 Hz, 3H), 2.87 (d, J=3.3 Hz, 1H), 2.79-2.70 (m, 2H), 2.67 (d, J=4.6 Hz, 3H), 1.18 (q, J=7.4 Hz, 3H). LCMS calc. for C₂₈H₃₁ClF₃N₈O₅ [M+H]⁺: m/z=651.2; Found: 651.2.

### Example 330:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-methoxy-2-oxoacetamide

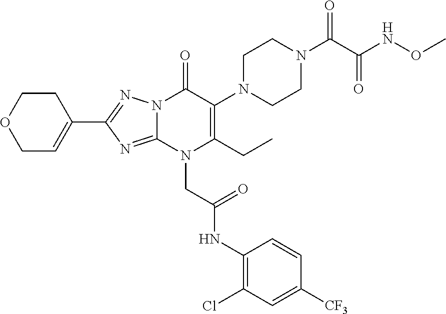 [SMILES: CCc1c(N2CCN(C(=O)C(=O)NOC)CC2)c(=O)n2nc(C3=CCOCC3)nc2n1CC(=O)Nc1ccc(C(F)(F)F)cc1Cl]

This compound was prepared by procedures analogous to those described for method A using 2-(methoxyamino)-2-oxoacetic acid to replace 4-(trifluoromethyl)-1H-imidazole-2-carboxylic acid to afford the title product as a white solid. ¹H NMR (400 MHZ, DMSO-d₆) δ ppm 11.97 (s, 1H), 10.37 (s, 1H), 8.06 (d, J=8.6 Hz, 1H), 7.97 (d, J=2.0 Hz, 1H), 7.72 (dd, J=8.7, 2.1 Hz, 1H), 6.83 (dd, J=3.5, 1.8 Hz, 1H), 5.32 (s, 2H), 4.32-4.22 (m, 3H), 3.80 (t, J=5.7 Hz, 3H), 3.66 (d, J=2.1 Hz, 3H), 3.50-3.38 (m, 2H), 3.31 (s, 2H), 3.02-2.86 (m, 3H), 2.77 (d, J=9.5 Hz, 3H), 1.19 (t, J=7.5 Hz, 3H). LCMS calc. for C₂₈H₃₁ClF₃N₈O₆ [M+H]⁺: m/z=667.2; Found: 667.0.

### Example 331:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(5-methyl-1H-pyrazol-3-yl)-2-oxoacetamide

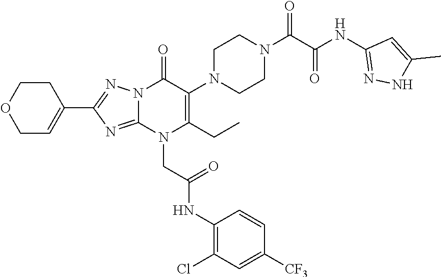 [SMILES: CCc1c(N2CCN(C(=O)C(=O)Nc3cc(C)[nH]n3)CC2)c(=O)n2nc(C3=CCOCC3)nc2n1CC(=O)Nc1ccc(C(F)(F)F)cc1Cl]

This compound was prepared by procedures analogous to those described for Method C using 2-((5-methyl-1H-pyrazol-3-yl)amino)-2-oxoacetic acid to replace benzoic acid to afford the title product as a white solid. ¹H NMR (400 MHZ, DMSO-d₆) δ ppm 12.14 (s, 1H), 11.05 (s, 1H), 10.36 (s, 1H), 8.06 (d, J=8.6 Hz, 1H), 7.97 (s, 1H), 7.72 (d, J=7.2 Hz, 1H), 6.83 (s, 1H), 6.29 (s, 1H), 5.32 (s, 2H), 4.29 (m, 3H), 3.80 (t, J=5.4 Hz, 2H), 3.68 (d, J=11.7 Hz, 1H), 3.57-3.37 (m, 4H), 3.27 (m, 2H), 3.07-2.95 (m, 2H), 2.80-2.71 (m, 2H), 2.21 (s, 3H), 1.19 (t, J=7.5 Hz, 3H). LCMS calc. for C₃₁H₃₃ClF₃N₁₀O₅ [M+H]⁺: m/z=717.2; Found: 717.2.

### Example 332:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-(thiazol-2-yl)acetamide

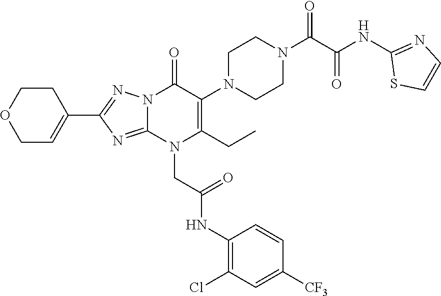 [SMILES: CCc1c(N2CCN(C(=O)C(=O)Nc3nccs3)CC2)c(=O)n2nc(C3=CCOCC3)nc2n1CC(=O)Nc1ccc(C(F)(F)F)cc1Cl]

This compound was prepared by procedures analogous to those described for Method K using 2-oxo-2-(thiazol-2-ylamino)acetic acid to replace 2-(dimethylamino)-2-oxoacetic acid to afford the title product as a white solid. ¹H NMR (400 MHZ, DMSO-d₆) δ ppm 13.02 (s, 1H), 10.36 (s, 1H), 8.06 (d, J=8.6 Hz, 1H), 7.96 (d, J=2.1 Hz, 1H), 7.72 (dd, J=8.7, 2.1 Hz, 1H), 7.55 (d, J=3.5 Hz, 1H), 7.35 (d, J=3.6 Hz, 1H), 6.83 (p, J=1.4 Hz, 1H), 5.32 (s, 2H), 4.35 (d, J=12.4 Hz, 1H), 4.25 (q, J=2.8 Hz, 2H), 3.80 (t, J=5.5 Hz, 2H), 3.64 (d, J=12.4 Hz, 1H), 3.49 (ddd, J=15.0, 11.6, 3.2 Hz, 3H), 3.31 (s, 2H), 2.98 (dt, J=13.2, 7.7 Hz, 3H), 2.77 (dd, J=26.6, 11.0 Hz, 2H), 1.19 (t, J=7.5 Hz, 3H). LCMS calc. for C₃₀H₃₀ClF₃N₉O₅S [M+H]⁺: m/z=720.2; Found: 720.1.

### Example 333:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-(1H-pyrazol-5-yl)acetamide

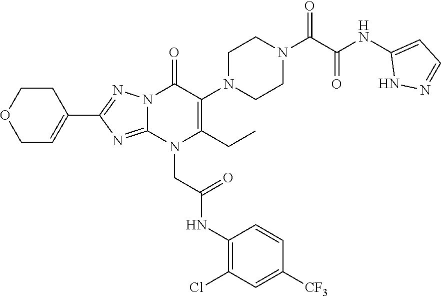 [SMILES: CCc1c(N2CCN(C(=O)C(=O)Nc3ccn[nH]3)CC2)c(=O)n2nc(C3=CCOCC3)nc2n1CC(=O)Nc1ccc(C(F)(F)F)cc1Cl]

This compound was prepared by procedures analogous to those described for Method K using 2-((1H-pyrazol-5-yl)amino)-2-oxoacetic acid to replace 2-(dimethylamino)-2-oxoacetic acid to afford the title product as a white solid. ¹H NMR (400 MHZ, DMSO-d₆) δ ppm 12.49 (s, 1H), 11.20 (s, 1H), 10.45-10.33 (m, 1H), 8.06 (d, J=8.6 Hz, 1H), 7.96 (d, J=2.0 Hz, 1H), 7.71 (dd, J=8.7, 2.1 Hz, 1H), 7.65 (d, J=2.4 Hz, 1H), 6.83 (td, J=3.0, 1.5 Hz, 1H), 6.51 (s, 1H), 5.32 (s, 2H), 4.36-4.30 (m, 1H), 4.25 (q, J=2.9 Hz, 2H), 3.80 (t, J=5.5 Hz, 2H), 3.71 (d, J=12.6 Hz, 1H), 3.56-3.43 (m, 3H), 3.32-3.30 (m, 2H), 3.02-2.88 (m, 3H), 2.76 (dd, J=17.9, 11.1 Hz, 2H), 1.19 (t, J=7.4 Hz, 3H). LCMS calc. for C₃₀H₃₁ClF₃N₁₀O₅ [M+H]⁺: m/z=703.2; Found: 703.2.

### Example 334:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-(thiazol-5-yl)acetamide

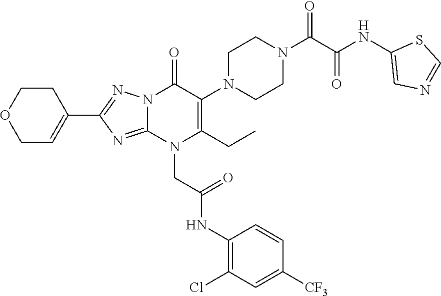 [SMILES: CCc1c(N2CCN(C(=O)C(=O)Nc3cncs3)CC2)c(=O)n2nc(C3=CCOCC3)nc2n1CC(=O)Nc1ccc(C(F)(F)F)cc1Cl]

This compound was prepared by procedures analogous to that described for Method K using 2-oxo-2-(thiazol-5-ylamino)acetic acid to replace 2-(dimethylamino)-2-oxoacetic acid to afford the title product as a white solid. ¹H NMR (400 MHZ, DMSO-d₆) δ ppm 10.41 (s, 1H), 8.65 (s, 1H), 8.06 (d, J=8.6 Hz, 1H), 7.96 (d, J=2.0 Hz, 1H), 7.78-7.66 (m, 2H), 6.87-6.79 (m, 1H), 5.32 (s, 2H), 4.39 (d, J=12.3 Hz, 1H), 4.25 (q, J=2.9 Hz, 2H), 3.94 (d, J=12.7 Hz, 1H), 3.80 (t, J=5.4 Hz, 2H), 3.47 (tt, J=11.6, 6.3 Hz, 4H), 3.31-3.30 (m, 1H), 2.99 (dt, J=13.0, 8.5 Hz, 3H), 2.78 (dd, J=26.5, 11.2 Hz, 2H), 1.21 (q, J=8.1, 7.5 Hz, 3H). LCMS calc. for C₃₀H₃₀ClF₃N₉O₅S [M+H]⁺: m/z=720.2; Found: 720.2.

### Example 335:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-(thiazol-4-yl)acetamide

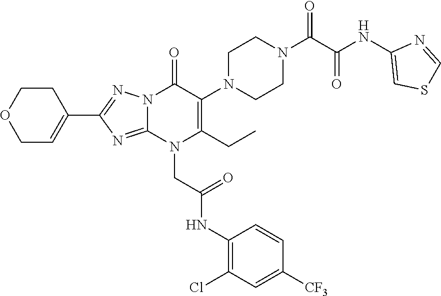 [SMILES: CCc1c(N2CCN(C(=O)C(=O)Nc3cscn3)CC2)c(=O)n2nc(C3=CCOCC3)nc2n1CC(=O)Nc1ccc(C(F)(F)F)cc1Cl]

This compound was prepared by procedures analogous to that described for Method K using 2-oxo-2-(thiazol-4-ylamino)acetic acid to replace 2-(dimethylamino)-2-oxoacetic acid to afford the title product as a white solid. ¹H NMR (400 MHZ, DMSO-d₆) δ ppm 11.86 (s, 1H), 10.40 (s, 1H), 9.02 (d, J=2.2 Hz, 1H), 8.06 (d, J=8.5 Hz, 1H), 7.94 (s, 1H), 7.74-7.66 (m, 2H), 6.83 (dd, J=3.3, 1.7 Hz, 1H), 5.31 (s, 2H), 4.34 (d, J=12.4 Hz, 1H), 4.25 (q, J=2.8 Hz, 2H), 3.80 (t, J=5.5 Hz, 2H), 3.68 (d, J=12.6 Hz, 1H), 3.57-3.45 (m, 3H), 3.10-2.87 (m, 4H), 2.76 (dd, J=23.5, 11.3 Hz, 3H), 1.19 (t, J=7.5 Hz, 3H). LCMS calc. for C₃₀H₃₀ClF₃N₉O₅S [M+H]⁺: m/z=720.2; Found: 720.1.

### Example 336:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-ethyl-2-oxoacetamide

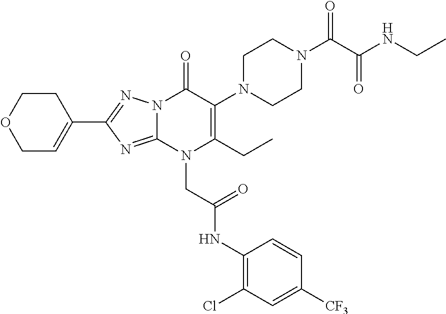 [SMILES: CCNC(=O)C(=O)N1CCN(c2c(CC)n(CC(=O)Nc3ccc(C(F)(F)F)cc3Cl)c3nc(C4=CCOCC4)nn3c2=O)CC1]

This compound was prepared by procedures analogous to that described for method A using INT B1 and 2-(ethylamino)-2-oxoacetic acid to afford the title product as a white solid. ¹H NMR (400 MHz, DMSO-d₆) δ ppm 10.36 (s, 1H), 8.74 (t, J=5.7 Hz, 1H), 8.07 (dd, J=8.6, 6.0 Hz, 1H), 7.97 (d, J=2.1 Hz, 1H), 7.72 (dd, J=8.7, 2.2 Hz, 1H), 6.83 (p, J=1.6 Hz, 1H), 5.32 (d, J=2.1 Hz, 2H), 4.32 (d, J=12.8 Hz, 1H), 4.26 (p, J=3.3, 2.9 Hz, 2H), 3.84 (d, J=12.1 Hz, 1H), 3.81 (d, J=5.3 Hz, 2H), 3.48-3.40 (m, 2H), 3.28-3.24 (m, 1H), 3.16 (ttd, J=7.3, 5.5, 1.6 Hz, 2H), 3.00 (dd, J=11.3, 4.6 Hz, 2H), 2.91-2.84 (m, 1H), 2.76 (d, J=13.2 Hz, 2H), 2.52 (s, 2H), 1.19 (t, J=7.4 Hz, 3H), 1.06 (t, J=7.2 Hz, 3H). LCMS calc. for C₂₉H₃₂ClF₃N₈O₅ [M+H]⁺: m/z=665.2; Found: 665.0.

### Example 337: N-(Benzyloxy)-2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoacetamide

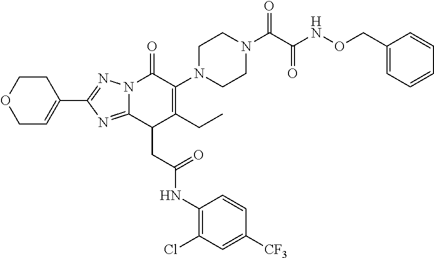 [SMILES: CCC1=C(N2CCN(C(=O)C(=O)NOCc3ccccc3)CC2)C(=O)n2nc(C3=CCOCC3)nc2C1CC(=O)Nc1ccc(C(F)(F)F)cc1Cl]

This compound was prepared by procedures analogous to that described for method A using INT B1 and 2-((benzyloxy)amino)-2-oxoacetic acid to afford the title product as a white solid. ¹H NMR (400 MHZ, DMSO-d₆) δ ppm 12.00 (s, 1H), 10.37 (s, 1H), 8.06 (d, J=8.6 Hz, 1H), 7.97 (d, J=2.0 Hz, 1H), 7.72 (dd, J=8.8, 2.2 Hz, 1H), 7.44-7.34 (m, 5H), 6.83 (s, 1H), 5.32 (s, 2H), 4.92-4.83 (m, 2H), 4.33-4.19 (m, 3H), 3.80 (t, J=5.6 Hz, 2H), 3.52 (d, J=12.6 Hz, 1H), 3.39 (d, J=12.5 Hz, 4H), 3.24-3.13 (m, 1H), 2.98 (t, J=8.0 Hz, 2H), 2.86 (td, J=12.6, 3.4 Hz, 1H), 2.70 (dd, J=37.0, 10.8 Hz, 2H), 1.18 (t, J=7.4 Hz, 3H). LCMS calc. for C₃₄H₃₅ClF₃N₈O₆ [M+H]⁺: m/z=743.2; Found: 743.2.

Example 338: N-(2-Chloro-4-(trifluoromethyl)phenyl)-2-(2-(3,6-dihydro-2H-pyran-4-yl)-5-ethyl-7-oxo-6-(4-((2-(tetrahydrofuran-2-yl) acetyl)glycyl)piperazin-1-yl)-[1,2,4]triazolo[1,5-a]pyrimidin-4(7H)-yl)acetamide

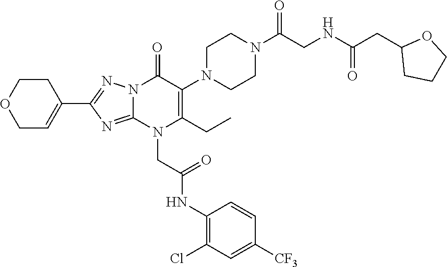 [SMILES: CCc1c(N2CCN(C(=O)CNC(=O)CC3CCCO3)CC2)c(=O)n2nc(C3=CCOCC3)nc2n1CC(=O)Nc1ccc(C(F)(F)F)cc1Cl]

This compound was prepared using procedures analogous to those described for Method H using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-(tetrahydrofuran-2-yl)acetic acid to afford the title product as a white solid. ¹H NMR (400 MHZ, DMSO-d₆) δ ppm 10.37 (s, 1H), 8.06 (d, J=8.4 Hz, 1H), 7.97 (d, J=1.8 Hz, 2H), 7.76-7.69 (m, 1H), 6.83 (s, 1H), 5.32 (s, 2H), 4.37 (d, J=12.2 Hz, 1H), 4.25 (d, J=2.4 Hz, 2H), 4.10-4.05 (m, 1H), 4.00 (d, J=5.1 Hz, 1H), 3.88-3.78 (m, 3H), 3.77-3.71 (m, 1H), 3.59 (dd, J=14.2, 7.8 Hz, 1H), 3.47 (t, J=10.8 Hz, 1H), 3.36-3.34 (m, 4H), 3.21-3.19 (m, 1H), 2.99-2.97 (m, 2H), 2.79-2.77 (m, 1H), 2.73-2.70 (m, 2H), 2.44-2.42 (m, 1H), 2.33-2.24 (m, 1H), 1.95-1.93 (m, 1H), 1.82-1.80 (m, 2H), 1.58-1.45 (m, 1H), 1.19-1.17 (m, 3H). LCMS calc. for C₃₃H₃₉ClF₃N₈O₆ [M+H]⁺: m/z=735.3; Found: 735.6.

### Example 339: N-(2-Chloro-4-(trifluoromethyl)phenyl)-2-(2-(3,6-dihydro-2H-pyran-4-yl)-5-ethyl-7-oxo-6-(4-((2-(pyrrolidin-1-yl) acetyl)glycyl)piperazin-1-yl)-[1,2,4]triazolo[1,5-a]pyrimidin-4(7H)-yl)acetamide

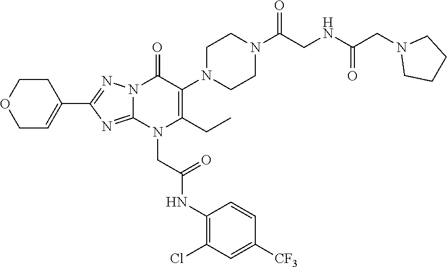 [SMILES: CCc1c(N2CCN(C(=O)CNC(=O)CN3CCCC3)CC2)c(=O)n2nc(C3=CCOCC3)nc2n1CC(=O)Nc1ccc(C(F)(F)F)cc1Cl]

This compound was prepared using procedures analogous to those described for Method H using N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38) and 2-(pyrrolidin-1-yl)acetic acid to afford the title product as a white solid. ¹H NMR (400 MHZ, DMSO-d₆) 0 ppm 10.37 (s, 1H), 8.15-8.05 (m, 1H), 8.01 (d, J=9.8 Hz, 2H), 7.73 (d, J=8.8 Hz, 1H), 6.83 (s, 1H), 5.32 (s, 2H), 4.38 (d, J=12.8 Hz, 1H), 4.26 (s, 2H), 4.11-4.09 (m, 1H), 4.05-3.96 (m, 1H), 3.83-3.81 (m, 3H), 3.48-3.46 (m, 2H), 3.36-3.34 (m, 3H), 3.23-3.21 (m, 2H), 2.99-2.97 (m, 2H), 2.80-2.78 (m, 2H), 2.74-2.64 (m, 5H), 1.78-1.76 (m, 4H), 1.19-1.17 (m, 3H). LCMS calc. for C₃₃H₄₀ClF₃N₉O₅ [M+H]⁺: m/z=734.3; Found: 734.6.

### Example 340:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(pyrimidin-4-yl)acetamide

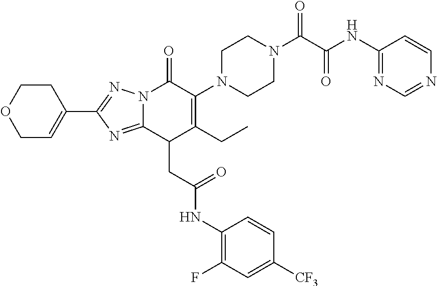 [SMILES: CCC1=C(N2CCN(C(=O)C(=O)Nc3ccncn3)CC2)C(=O)n2nc(C3=CCOCC3)nc2C1CC(=O)Nc1ccc(C(F)(F)F)cc1F]

To a mixture of INT B13 (30 mg, 0.05 mmol) in DMF (1 mL) was added 2-oxo-2-(pyrimidin-4-ylamino)acetic acid (14 mg, 0.08 mmol), NMI (13 mg, 0.16 mmol), TCFH (23 mg, 0.08 mmol). The mixture was stirred at r.t. for 2 h., and then concentrated under reduced pressure. The residue was purified by Prep-HPLC on a C18 column eluting with MeCN/H₂O (50%) to afford the title compound (9.4 mg, 25% yield) as a white solid. LCMS calc. for C₃₁H₃₁F₄N₁₀O₅ [M+H]⁺: m/z=699.2; Found: 699.2.

### Example 341:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(isoxazol-5-yl)-2-oxoacetamide

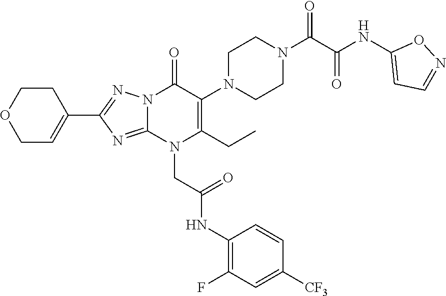 [SMILES: CCc1c(N2CCN(C(=O)C(=O)Nc3ccno3)CC2)c(=O)n2nc(C3=CCOCC3)nc2n1CC(=O)Nc1ccc(C(F)(F)F)cc1F]

To a mixture of INT B13 (50 mg, 0.09 mmol) in pyridine (1 mL) was added 2-(isoxazol-5-ylamino)-2-oxoacetic acid (INT C69, 21 mg, 0.14 mmol), EDCI (26 mg, 0.14 mmol). The mixture was stirred at r.t. for 2 h., and then concentrated under reduced pressure. The residue was purified by Prep-HPLC on a C18 column eluting with MeCN/H₂O (50%) to afford the title compound (5.1 mg, 21% yield) as a white solid. LCMS calc. for C₃₀H₃₀F₄N₉O₆ [M+H]⁺: m/z=688.2; Found: 688.1. Example 342:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(1,3,4-thiadiazol-2-yl)acetamide

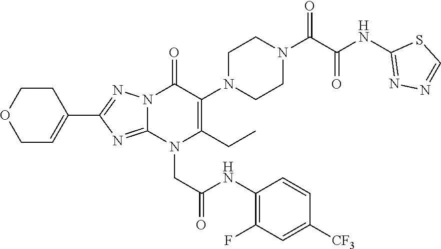 [SMILES: CCc1c(N2CCN(C(=O)C(=O)Nc3nncs3)CC2)c(=O)n2nc(C3=CCOCC3)nc2n1CC(=O)Nc1ccc(C(F)(F)F)cc1F]

To a mixture of INT B13 (30 mg, 0.05 mmol), 2-((1,3,4-thiadiazol-2-yl)amino)-2-oxoacetic acid (10 mg, 0.05 mmol) in pyridine (0.3 mL) was added EDCI (15 mg, 0.08 mmol) at r.t. under nitrogen atmosphere. The mixture was stirred at r.t. for 12 h. The reaction mixture was purified by flash chromatography on a C18 column eluting with MeCN/H₂O (45-65%) to afford the title compound (20.7 mg) as a white solid. LCMS calc. for C₂₉H₂₇F₄N₁₀O₅S [M−H]⁻: m/z=703.2; Found: 703.2.

### Example 343: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydropyrazolo[1,5-a]pyrimidin-6-yl)piperazin-1-yl)-2-oxoethyl)-2-fluorobenzamide

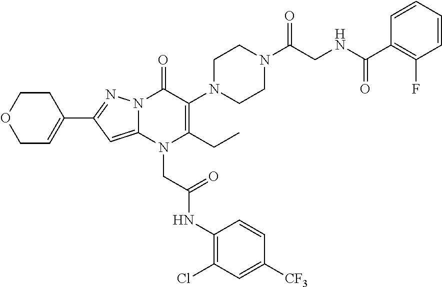 [SMILES: CCc1c(N2CCN(C(=O)CNC(=O)c3ccccc3F)CC2)c(=O)n2nc(C3=CCOCC3)cc2n1CC(=O)Nc1ccc(C(F)(F)F)cc1Cl]

To a mixture of INT B3 (80 mg, 0.14 mmol), (2-fluorobenzoyl)glycine (54 mg, 0.28 mmol), HOBt (38 mg, 0.27 mmol) and DIEA (70 mg, 0.54 mmol) in DMF (1 mL) was added EDCI (52 mg, 0.27 mmol). The mixture was stirred at r.t. for 2 h., and then concentrated under reduced pressure. The residue was purified by flash chromatography on a C18 column eluting with MeCN/H₂O (40%-50%) to afford the title compound (63 mg, 60% yield) as an off-white solid. LCMS calc. for C₃₅H₃₅ClF₄N₇O₅ [M+H]⁺: m/z=744.2; Found: 744.3.

Example 344: N-(2-(4-(4-(2-((2-chloro-4-(trifluoromethyl)phenyl)amino)-2-oxoethyl)-5-ethyl-2-morpholino-7-oxo-4,7-dihydropyrazolo[1,5-a]pyrimidin-6-yl)piperazin-1-yl)-2-oxoethyl)-2-fluorobenzamide

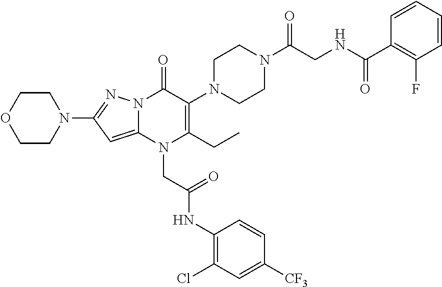 [SMILES: CCc1c(N2CCN(C(=O)CNC(=O)c3ccccc3F)CC2)c(=O)n2nc(N3CCOCC3)cc2n1CC(=O)Nc1ccc(C(F)(F)F)cc1Cl]

This compound was prepared using procedures analogous to those described for method I using INT B25 and (2-fluorobenzoyl)glycine to afford the title product as an off-white solid. LCMS calc. for C₃₄H₃₆ClF₄N₈O₅ [M+H]⁺: m/z=747.3; Found: 747.2.

### Example 345: N-(2-chloro-4-(trifluoromethyl)phenyl)-2-(2-(3,6-dihydro-2H-pyran-4-yl)-6-(4-(2-(1,3-dioxoisoindolin-2-yl)-2-fluoroacetyl)piperazin-1-yl)-5-ethyl-7-oxo-[1,2,4]tria zolo[1,5-a]pyrimidin-4(7H)-yl)acetamide

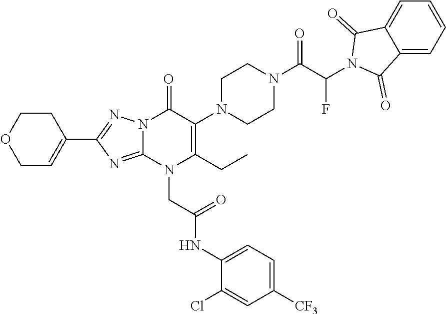 [SMILES: CCc1c(N2CCN(C(=O)C(F)N3C(=O)c4ccccc4C3=O)CC2)c(=O)n2nc(C3=CCOCC3)nc2n1CC(=O)Nc1ccc(C(F)(F)F)cc1Cl]

This compound was prepared using procedures analogous to those described for Method H using 2-(1,3-dioxoisoindolin-2-yl)-2-fluoroacetic acid and INT B 1 to afford the title product as a white solid. ¹H NMR (400 MHZ, DMSO-d₆) δ ppm 10.33 (s, 1H), 8.09-7.93 (m, 6H), 7.71 (d, J=7.5 Hz, 1H), 6.99 (dd, J=47.4, 14.0 Hz, 1H), 6.82 (s, 1H), 5.29 (s, 2H), 4.49-4.31 (m, 1H), 4.25 (s, 2H), 3.80 (s, 2H), 3.75-3.39 (m, 3H), 3.11-2.56 (m, 8H), 1.14 (dt, J=14.8, 7.5 Hz, 3H). LCMS calc. for C₃₅H₃₂ClF₄N₈O₆ [M+H]⁺: m/z=771.2; Found: 771.2.

### Example 346: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-4-methylthiazole-2-carboxamide

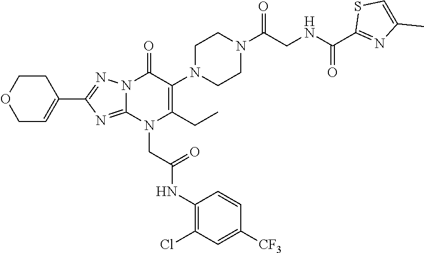 [SMILES: CCc1c(N2CCN(C(=O)CNC(=O)c3nc(C)cs3)CC2)c(=O)n2nc(C3=CCOCC3)nc2n1CC(=O)Nc1ccc(C(F)(F)F)cc1Cl]

To a mixture of 4-methylthiazole-2-carboxylic acid (14 mg, 0.1 mmol), N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (Example 38, 31 mg, 0.05 mmol) and pyridine (40 mg, 0.5 mmol) was added POCl₃ (45 mg, 0.3 mmol), and then stirred at r.t. for 2 h. The mixture was diluted with MeOH (3 mL) and concentrated under reduced pressure. The residue was purified by flash chromatography on a C18 column eluting with MeCN/H₂O (40-55% with 0.1% NH₄HCO₃) to afford the title compound (19 mg) as a white solid. LCMS calc. for C₃₂H₃₂ClF₃N₉O₅S [M−H]⁻: m/z=746.2; Found: 746.1.

### Example 347:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(4-methylthiazol-2-yl)-2-oxoacetamide

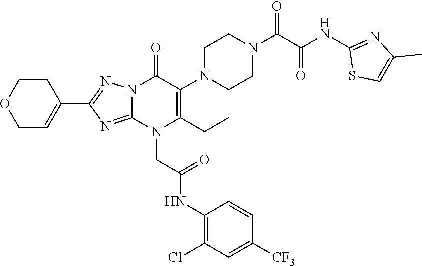 [SMILES: CCc1c(N2CCN(C(=O)C(=O)Nc3nc(C)cs3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide (INT B1, 50 mg, 0.09 mmol) and 2-((4-methylthiazol-2-yl)amino)-2-oxoacetic acid (19 mg, 0.1 mmol, INT C71) in DMF (0.5 mL) was added EDCI (27 mg, 0.15 mmol), HOBt (20 mg, 0.15 mmol) and TEA (101 mg, 1 mmol). The reaction mixture was stirred at r.t. overnight, and then concentrated under reduced pressure. The residue was purified by prep-HPLC on a C18 column eluting with MeCN/H₂O (40-50%) to afford the title compound (26.9 mg) as a white solid. ¹H NMR (600 MHZ, DMSO-d₆) δ ppm 12.60 (s, 1H), 10.40 (s, 1H), 8.65 (s, 1H), 8.06 (d, J=8.4 Hz, 1H), 7.98 (d, J=1.8 Hz, 1H), 7.74 (d, J=8.4 Hz, 1H), 7.35 (s, 1H), 6.84 (s, 1H), 6.23 (s, 1H), 5.33 (s, 2H), 4.35 (d, J=4.8 Hz, 1H), 4.25 (s, 2H), 3.80 (t, J=5.4 Hz, 2H), 3.74 (d, J=5.4 Hz, 1H), 3.53-3.43 (m, 2H), 3.40-3.28 (m, 3H), 3.10-2.90 (m, 3H), 2.79 (dd, J=26.0, 12.0 Hz, 2H), 2.02 (s, 3H), 1.21 (t, J=7.2 Hz, 3H). LCMS calc. for C₃₁H₃₀ClF₃N₉O₅S [M−H]⁻: m/z=732.2; Found: 732.1.

### Example 348:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(3-methylisoxazol-5-yl)-2-oxoacetamide

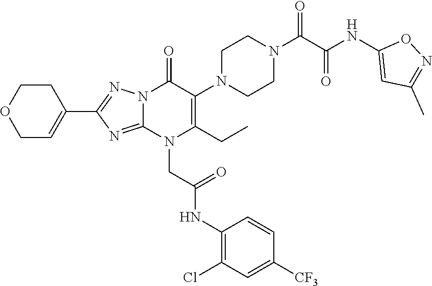 [SMILES: CCc1c(N2CCN(C(=O)C(=O)Nc3cc(C)no3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide (INT B1, 50 mg, 0.09 mmol) in pyridine (1 mL) was added 2-((3-methylisoxazol-5-yl)amino)-2-oxoacetic acid (23 mg, 0.13 mmol, INT C70) and EDCI (26 mg, 0.13 mmol). The mixture was stirred at r.t. for 2 h., and then concentrated under reduced pressure. The residue was purified by prep-HPLC on a C18 column eluting with MeCN/H₂O (55-60%) to afford the title compound (9.7 mg) as a white solid. ¹H NMR (600 MHz, DMSO-d⁶) δ ppm 12.58 (s, 1H), 10.41 (s, 1H), 8.65 (s, 1H), 8.06 (d, J=8.4 Hz, 1H), 7.97 (d, J=1.8 Hz, 1H), 7.73 (d, J=8.4 Hz, 1H), 6.84 (s, 1H), 6.23 (s, 1H), 5.33 (s, 2H), 4.35 (d, J=4.8 Hz, 2H), 4.25 (s, 2H), 3.80 (t, J=5.4 Hz, 2H), 3.74 (d, J=5.4 Hz, 1H), 3.53-3.43 (m, 2H), 3.40-3.28 (m, 3H), 3.08-2.92 (m, J=3H), 2.78 (dd, J=26.0, 12.0 Hz, 2H), 2.21 (s, 3H), 1.20 (t, J=7.2 Hz, 3H). LCMS calc. for C₃₁H₃₀ClF₃N₉O₆ [M−H]⁻: m/z=716.2; Found: 716.1.

### Example 349:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-2-oxo-N-(thiazol-5-yl)acetamide

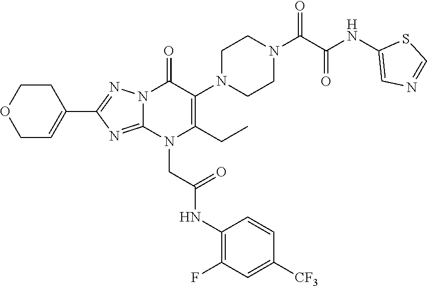 [SMILES: CCc1c(N2CCN(C(=O)C(=O)Nc3cncs3)CC2)c(=O)n2nc(C3=CCOCC3)nc2n1CC(=O)Nc1ccc(C(F)(F)F)cc1F]

To a mixture of 2-oxo-2-(thiazol-5-ylamino)acetic acid (INT C49, 26 mg, 0.15 mmol) and 2-(2-(3,6-dihydro-2H-pyran-4-yl)-5-ethyl-7-oxo-6-(piperazin-1-yl)-[1,2,4]triazolo[1,5-a]pyrimidin-4(7H)-yl)-N-(2-fluoro-4-(trifluoromethyl)phenyl)acetamide (INT B13, 55 mg, 0.1 mmol) in DMF (1 mL) was added EDCI (27 mg, 0.15 mmol), HOBt (20 mg, 0.15 mmol) and TEA (101 mg, 1 mmol). The reaction mixture was stirred at r.t. overnight, and then concentrated under reduced pressure. The residue was purified by prep-HPLC on a C18 column eluting with MeCN/H₂O (40-50%) to afford the title compound (44.8 mg) as a white solid. LCMS calc. for C₃₀H₂₈F₄N₉O₅S [M−H]⁻: m/z=702.2; Found: 702.2.

### Example 350:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(4-methylthiazol-2-yl)-2-oxoacetamide

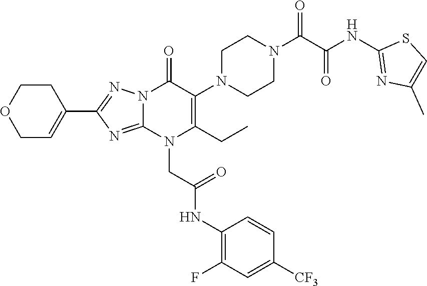 [SMILES: CCc1c(N2CCN(C(=O)C(=O)Nc3nc(C)cs3)CC2)c(=O)n2nc(C3=CCOCC3)nc2n1CC(=O)Nc1ccc(C(F)(F)F)cc1F]

To a mixture of 2-((4-methylthiazol-2-yl)amino)-2-oxoacetic acid (28 mg, 0.15 mmol, INT C71) and INT B13 (55 mg, 0.1 mmol) in DMF (1 mL) was added EDCI (27 mg, 0.15 mmol), HOBt (20 mg, 0.15 mmol) and TEA (101 mg, 1 mmol). The mixture was stirred at r.t. overnight, and then concentrated under reduced pressure. The residue was purified by prep-HPLC on a C18 column eluting with MeCN/H₂O (40-50%) to afford the title compound (18.2 mg) as a white solid. ¹H NMR (650 MHz, DMSO-d₆) δ ppm 12.58 (s, 1H), 10.71 (s, 1H), 8.23 (d, J=8.4 Hz, 1H), 7.83-7.75 (m, 1H), 7.56 (d, J=8.4 Hz, 1H), 7.34 (s, 1H), 6.80 (s, 1H), 6.23 (s, 1H), 5.30 (s, 2H), 4.37 (d, J=4.8 Hz, 1H), 4.25 (s, 2H), 3.85 (t, J=5.4 Hz, 2H), 3.78 (d, J=5.4 Hz, 1H), 3.58-3.33 (m, 5H), 3.13-2.91 (m, 3H), 2.80 (dd, J=26.0, 12.0 Hz, 2H), 2.05 (s, 3H), 1.17 (t, J=7.4 Hz, 3H). LCMS calc. for C₃₁H₃₀F₄N₉O₅S [M−H]⁻: m/z=716.2; Found: 716.1.

### Example 351: N-(5-Chloro-1,3,4-thiadiazol-2-yl)-2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoacetamide

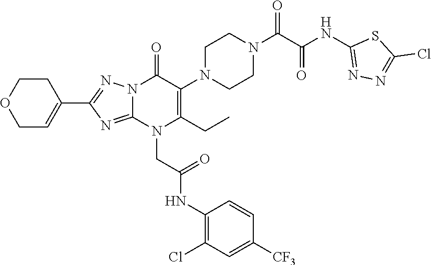 [SMILES: CCc1c(N2CCN(C(=O)C(=O)Nc3nnc(Cl)s3)CC2)c(=O)n2nc(C3=CCOCC3)nc2n1CC(=O)Nc1ccc(C(F)(F)F)cc1Cl]

To a solution of INT B1 (50 mg, 0.09 mmol) in DMF (0.5 mL) was added 2-((5-chloro-1,3,4-thiadiazol-2-yl)amino)-2-oxoacetic acid (28 mg, 0.13 mmol, INT C72), NMI (22 mg, 0.27 mmol) and TCFH (37 mg, 0.13 mmol). The mixture was stirred at r.t. for 2 h., and then concentrated under reduced pressure. The residue was purified by prep-HPLC on a C18 column eluting with MeCN/H₂O (35-40%) to afford the title compound (50 mg) as a white solid. LCMS calc. for C₂₉H₂₆C₁₂F₃N₁₀O₅S [M−H]⁻: m/z=753.1; Found: 753.0.

### Example 352:2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-N-(5-methylisoxazol-3-yl)-2-oxoacetamide

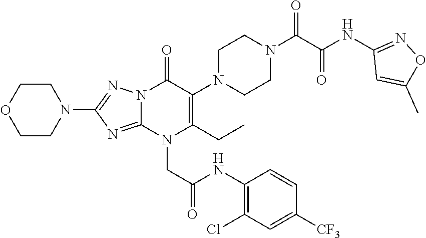 [SMILES: CCc1c(N2CCN(C(=O)C(=O)Nc3cc(C)on3)CC2)c(=O)n2nc(N3CCOCC3)nc2n1CC(=O)Nc1ccc(C(F)(F)F)cc1Cl]

To a mixture of INT B4 (30 mg, 0.05 mmol), 2-((5-methylisoxazol-3-yl)amino)-2-oxoacetic acid (8.9 mg, 0.05 mmol) and TCFH (25 mg, 0.09 mmol) in MeCN (0.3 mL) was added NMI (16 mg, 0.2 mmol) at r.t. The mixture was stirred at r.t. overnight, and then concentrated under reduced pressure. The residue was purified by flash chromatography on a C18 column eluting with MeCN/H₂O (45-65%) to afford the title compound (12 mg) as a white solid. LCMS calc. for C₃₀H₃₁ClF₃N₁₀O₆ [M−H]⁻: m/z=719.2; Found: 719.1.

Example 353:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(4-methyloxazol-2-yl)-2-oxoacetamide

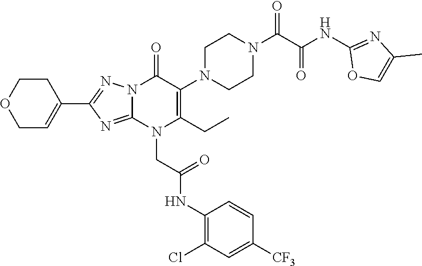 [SMILES: CCc1c(N2CCN(C(=O)C(=O)Nc3nc(C)co3)CC2)c(=O)n2nc(C3=CCOCC3)nc2n1CC(=O)Nc1ccc(C(F)(F)F)cc1Cl]

To a mixture of INT B1 (30 mg, 0.05 mmol), 2-((4-methyloxazol-2-yl)amino)-2-oxoacetic acid (17 mg, 0.1 mmol, INT C73) in DMF (1 mL) was added EDCI (27 mg, 0.15 mmol), HOBt (20 mg, 0.15 mmol) and TEA (101 mg, 1 mmol) at r.t. The mixture was stirred at r.t. overnight, and then concentrated under reduced pressure. The residue was purified by prep-HPLC on a C18 column eluting with MeCN/H₂O (55-65%) to afford the title compound (5.7 mg) as a white solid. LCMS calc. for C₃₁H₃₀ClF₃N₉O₆ [M−H]⁻: m/z=716.2; Found: 716.1.

### Example 354:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-(isoxazol-5-yl)-2-oxoacetamide

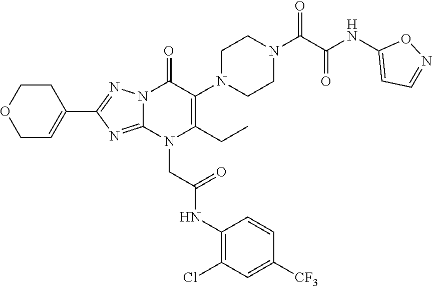 [SMILES: CCc1c(N2CCN(C(=O)C(=O)Nc3ccno3)CC2)c(=O)n2nc(C3=CCOCC3)nc2n1CC(=O)Nc1ccc(C(F)(F)F)cc1Cl]

To a solution of INT B1 (50 mg, 0.09 mmol) in pyridine (1 mL) was added 2-(isoxazol-5-ylamino)-2-oxoacetic acid (21 mg, 0.13 mmol, INT C69), EDCI (25 mg, 0.13 mmol). The mixture was stirred at r.t. for 2 h., and then concentrated under reduced pressure. The residue was purified by prep-HPLC on a C18 column eluting with MeCN/H₂O (40-50%) to afford the title compound (2.0 mg) as an off-white solid. LCMS calc. for C₃₀H₂₈ClF₃N₉O₆ [M−H]⁻: m/z=702.2; Found: 702.1.

Example 355:2-(4-(2-(3,6-Dihydro-2H-pyran-4-yl)-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazin-1-yl)-N-(4-methyloxazol-2-yl)-2-oxoacetamide

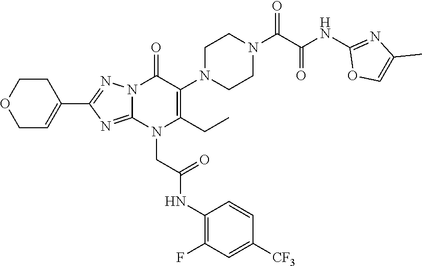 [SMILES: CCc1c(N2CCN(C(=O)C(=O)Nc3nc(C)co3)CC2)c(=O)n2nc(C3=CCOCC3)nc2n1CC(=O)Nc1ccc(C(F)(F)F)cc1F]

To a mixture of 2-((4-methyloxazol-2-yl)amino)-2-oxoacetic acid (26 mg, 0.15 mmol) and INT B13 (55 mg, 0.1 mmol) in DMF (1 mL) was added EDCI (27 mg, 0.15 mmol), HOBt (20 mg, 0.15 mmol) and TEA (101 mg, 1 mmol). The mixture was stirred at r.t. overnight, and then concentrated under reduced pressure. The residue was purified by prep-HPLC on a C18 column eluting with MeCN/H₂O (40-50%) to afford the title compound (45 mg) as a white solid. LCMS calc. for C₃₁H₃₀F₄N₉O₆ [M−H]⁻: m/z=700.2; Found: 700.1.

### Example 356: N-(2-Chloro-4-(trifluoromethyl)phenyl)-2-(2-(3,6-dihydro-2H-pyran-4-yl)-5-ethyl-6-(4-(2-morpholino-2-oxoacetyl)piperazin-1-yl)-7-oxo-[1,2,4]triazolo[1,5-a]pyrimidin-4(7H)-yl)acetamide

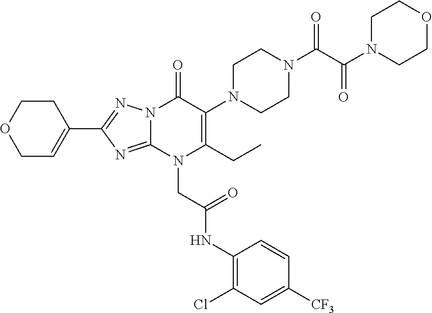 [SMILES: CCc1c(N2CCN(C(=O)C(=O)N3CCOCC3)CC2)c(=O)n2nc(C3=CCOCC3)nc2n1CC(=O)Nc1ccc(C(F)(F)F)cc1Cl]

To a solution of INT B1 (50 mg, 0.09 mmol) in DMF (0.5 mL) was added 2-morpholino-2-oxoacetic acid (21 mg, 0.13 mmol, INT C74), NMI (22 mg, 0.27 mmol) and TCFH (37 mg, 0.13 mmol). The mixture was stirred at r.t. for 2 h., and then concentrated under reduced pressure. The residue was purified by prep-HPLC on a C18 column eluting with MeCN/H₂O (50%) to afford the title compound (5.1 mg, 41% yield) as a white solid. LCMS calc. for C₃₁H₃₅ClF₃N₈O₆ [M+H]⁺: m/z=707.2; Found: 707.1.

Example 357:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-cyclopentyl-2-oxoacetamide

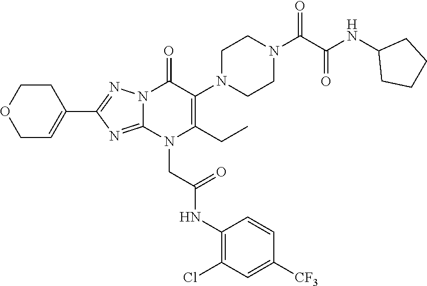 [SMILES: CCc1c(N2CCN(C(=O)C(=O)NC3CCCC3)CC2)c(=O)n2nc(C3=CCOCC3)nc2n1CC(=O)Nc1ccc(C(F)(F)F)cc1Cl]

To a solution of INT B1 (50 mg, 0.09 mmol) in DMF (0.5 mL) was added 2-(cyclopentylamino)-2-oxoacetic acid (21 mg, 0.13 mmol), NMI (22 mg, 0.27 mmol) and TCFH (37 mg, 0.13 mmol). The mixture was stirred at r.t. for 2 h., and then concentrated under reduced pressure. The residue was purified by prep-HPLC on a C18 column eluting with MeCN/H₂O (40-50%) to afford the title compound (14.6 mg) as a white solid. LCMS calc. for C₃₂H₃₅ClF₃N₈O₅ [M−H]⁻: m/z=703.2; Found: 703.2.

### Example 358: N-(2-Chloro-4-(trifluoromethyl)phenyl)-2-(2-(3,6-dihydro-2H-pyran-4-yl)-5-ethyl-7-oxo-6-(4-(2-oxo-2-(pyrrolidin-1-yl) acetyl)piperazin-1-yl)-[1,2,4]triazolo[1,5-a]pyrimidin-4(7H)-yl)acetamide

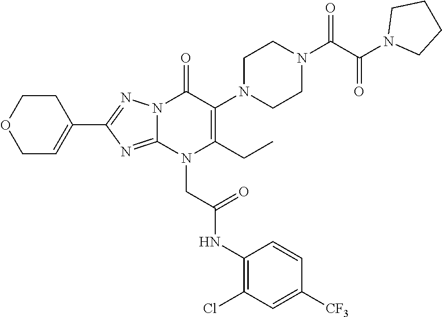 [SMILES: CCc1c(N2CCN(C(=O)C(=O)N3CCCC3)CC2)c(=O)n2nc(C3=CCOCC3)nc2n1CC(=O)Nc1ccc(C(F)(F)F)cc1Cl]

To a mixture of INT B1 (30 mg, 0.05 mmol), 2-oxo-2-(pyrrolidin-1-yl)acetic acid (7.6 mg, 0.05 mmol) and TCFH (22.3 mg, 0.08 mmol) in MeCN (0.5 mL) was added NMI (22 mg, 0.25 mmol) at r.t. The mixture was stirred at r.t. for 12 h., and then concentrated under reduced pressure. The residue was purified by flash chromatography on a C18 column eluting with MeCN/H₂O (45-65%) to afford the title compound (25.5 mg) as a white solid. LCMS calc. for C₃₁H₃₃ClF₃N₈O₅ [M−H]⁻: m/z=689.2; Found: 689.2.

Example 359:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-N-cyclopropyl-2-oxoacetamide

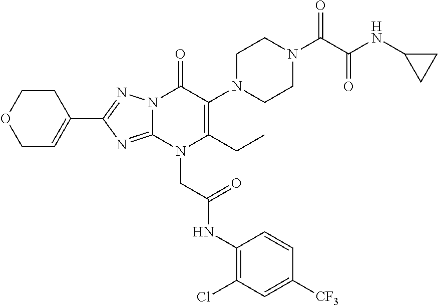 [SMILES: CCc1c(N2CCN(C(=O)C(=O)NC3CC3)CC2)c(=O)n2nc(C3=CCOCC3)nc2n1CC(=O)Nc1ccc(C(F)(F)F)cc1Cl]

To a solution of INT B1 (50 mg, 0.09 mmol) in DMF (0.5 mL) was added 2-(cyclopropylamino)-2-oxoacetic acid (17 mg, 0.13 mmol), NMI (22 mg, 0.27 mmol) and TCFH (37 mg, 0.13 mmol). The mixture was stirred at r.t. for 2 h., and then concentrated under reduced pressure. The residue was purified by prep-HPLC on a C18 column eluting with MeCN/H₂O (40-50%) to afford the title compound (37.2 mg) as a white solid. LCMS calc. for C₃₀H₃₁ClF₃N₈O₅ [M−H]⁻: m/z=675.2; Found: 675.2.

### Example 360:2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxo-N-(2,2,2-trifluoroethyl)acetamide

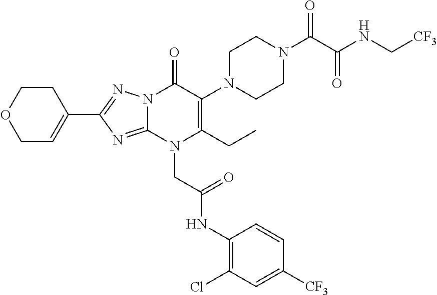 [SMILES: CCc1c(N2CCN(C(=O)C(=O)NCC(F)(F)F)CC2)c(=O)n2nc(C3=CCOCC3)nc2n1CC(=O)Nc1ccc(C(F)(F)F)cc1Cl]

To a mixture of INT B1 (30 mg, 0.05 mmol), 2-oxo-2-((2,2,2-trifluoroethyl)amino)acetic acid (9 mg, 0.05 mmol) and TCFH (22.3 mg, 0.08 mmol) in MeCN (0.3 mL) was added NMI (22 mg, 0.25 mmol) at r.t. The mixture was stirred at r.t. for 12 h., and then concentrated under reduced pressure. The residue was purified by flash chromatography on a C18 column eluting with MeCN/H₂O (45-65%) to afford the title compound (3.4 mg) as a white solid. LCMS calc. for C₂₉H₂₈ClF₆N₈O₅ [M−H]⁻: m/z=717.2; Found: 717.1.

Example 361: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoro-6-(methylamino) picolinamide

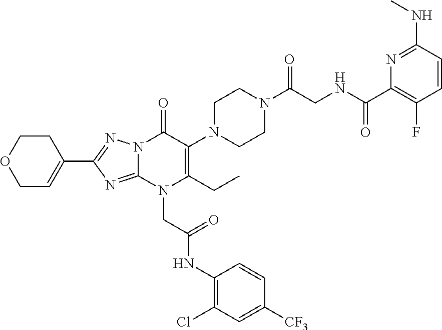 [SMILES: CCc1c(N2CCN(C(=O)CNC(=O)c3nc(NC)ccc3F)CC2)c(=O)n2nc(C3=CCOCC3)nc2n1CC(=O)Nc1ccc(C(F)(F)F)cc1Cl]

A mixture of (3-fluoro-6-(methylamino)picolinoyl)glycine (30 mg, 0.15 mmol, INT C75), INT B1 (56 mg, 0.1 mmol), EDCI (27 mg, 0.15 mmol), HOBt (20 mg, 0.15 mmol) and TEA (50 mg, 0.5 mmol) in DMF (1 mL) was stirred at r.t. overnight. The mixture was concentrated under reduced pressure. The residue was purified by flash chromatography on a C18 column eluting with MeCN/H₂O (35-40% with 0.1% TFA) to afford the title compound (17.9 mg) as a white solid. LCMS calc. for C₃₄H₃₄ClF₄N₁₀O₅ [M−H]⁻: m/z=773.2; Found: 773.2.

### Example 362: N-(2-Chloro-4-(trifluoromethyl)phenyl)-2-(2-(3,6-dihydro-2H-pyran-4-yl)-5-ethyl-7-oxo-6-(4-(pyrido[3,2-d]pyrimidin-4-ylglycyl)piperazin-1-yl)-[1,2,4]triazolo[1,5-a]pyrimidin-4(7H)-yl)acetamide

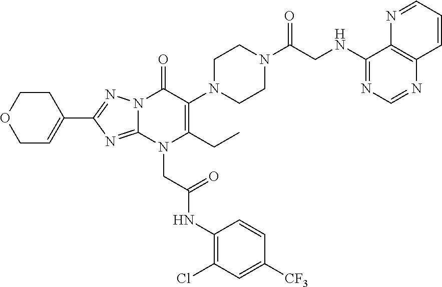 [SMILES: CCc1c(N2CCN(C(=O)CNc3ncnc4cccnc34)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38) and 4-chloropyrido[3,2-d]pyrimidine (8.3 mg, 0.05 mmol) in MeCN (0.5 mL) was added DIEA (19.1 mg, 0.15 mmol). The mixture was stirred at r.t. for 3 h., and then concentrated under reduced pressure. The residue was purified by flash chromatography on a C18 column eluting with MeCN/H₂O (45-60%) to afford the title compound (11.6 mg) as a white solid. LCMS calc. for C₃₄H₃₂ClF₃N₁₁O₄ [M−H]⁻: m/z=750.2; Found: 750.1.

Example 363:2-(6-(4-((7H-Pyrrolo[2,3-d]pyrimidin-4-yl)glycyl)piperazin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

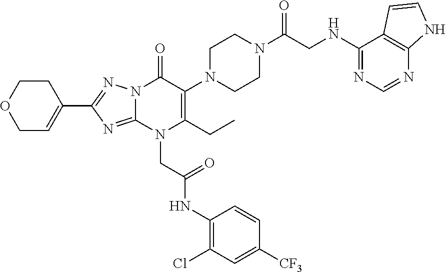 [SMILES: CCc1c(N2CCN(C(=O)CNc3ncnc4[nH]ccc34)CC2)c(=O)n2nc(C3=CCOCC3)nc2n1CC(=O)Nc1ccc(C(F)(F)F)cc1Cl]

To a mixture of INT B1 (30 mg, 0.05 mmol), (7H-pyrrolo[2,3-d]pyrimidin-4-yl)glycine (9.6 mg, 0.05 mmol, INT C76) and TCFH (22.3 mg, 0.08 mmol) in MeCN (0.3 mL) was added NMI (22 mg, 0.25 mmol) at r.t. The mixture was stirred at r.t. for 12 h., and then concentrated under reduced pressure. The reaction mixture was purified by flash chromatography on a C18 column eluting with MeCN/H₂O (40-65%) to afford the title compound (13.3 mg) as a white solid. LCMS calc. for C₃₃H₃₂ClF₃N₁₁O₄ [M−H]⁻: m/z=738.2; Found: 738.2.

### Example 364: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-hydroxypyrimidine-4-carboxamide

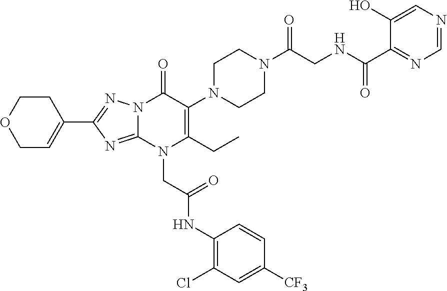 [SMILES: CCc1c(N2CCN(C(=O)CNC(=O)c3ncncc3O)CC2)c(=O)n2nc(C3=CCOCC3)nc2n1CC(=O)Nc1ccc(C(F)(F)F)cc1Cl]

A mixture of (5-hydroxypyrimidine-4-carbonyl)glycine (30 mg, 0.15 mmol, INT C77), INT B1 (56 mg, 0.1 mmol), EDCI (27 mg, 0.15 mmol), HOBt (20 mg, 0.15 mmol) and TEA (50 mg, 0.5 mmol) in DMF (1 mL) was stirred at r.t. overnight. The mixture was concentrated under reduced pressure. The residue was purified by flash chromatography on a C18 column eluting with MeCN/H₂O (35-45% with 0.1% TFA) to afford the title compound (27.4 mg) as a white solid. LCMS calc. for C₃₂H₃₁ClF₃N₁₀O₆ [M−H]⁻: m/z=743.2; Found: 743.1.

Example 365: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoro-6-oxo-1,6-dihydropyridine-2-carboxamide

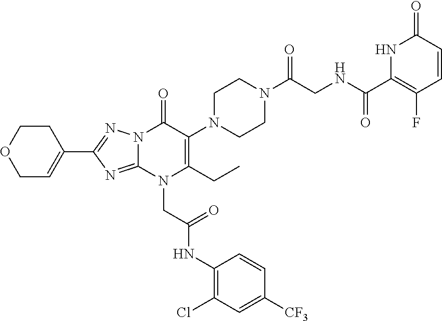 [SMILES: CCc1c(N2CCN(C(=O)CNC(=O)c3[nH]c(=O)ccc3F)CC2)c(=O)n2nc(C3=CCOCC3)nc2n1CC(=O)Nc1ccc(C(F)(F)F)cc1Cl]

A mixture of (3-fluoro-6-oxo-1,6-dihydropyridine-2-carbonyl)glycine (32 mg, 0.15 mmol, INT C78), INT B1 (56 mg, 0.1 mmol), EDCI (27 mg, 0.15 mmol), HOBt (20 mg, 0.15 mmol) and TEA (50 mg, 0.5 mmol) in DMF (1 mL) was stirred at r.t. overnight. The mixture was concentrated under reduced pressure. The residue was purified by flash chromatography on a C18 column eluting with MeCN/H₂O (35-50% with 0.1% TFA) to afford the title compound (24.6 mg) as a white solid. LCMS calc. for C₃₃H₃₁ClF₄N₉O₆ [M−H]⁻: m/z=760.2; Found: 760.1.

### Example 366: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-6-oxo-1,6-dihydropyridine-2-carboxamide

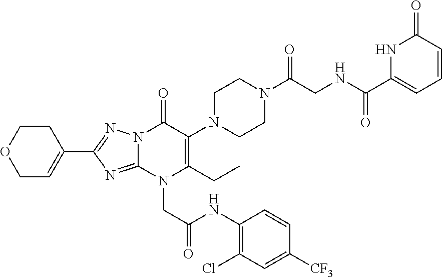 [SMILES: CCc1c(N2CCN(C(=O)CNC(=O)c3cccc(=O)[nH]3)CC2)c(=O)n2nc(C3=CCOCC3)nc2n1CC(=O)Nc1ccc(C(F)(F)F)cc1Cl]

A mixture of INT B1 (56 mg, 0.1 mmol) and 6-oxo-1,6-dihydropyridine-2-carboxylic acid (16 mg, 0.12 mmol) EDCI (27 mg, 0.15 mmol), HOBt (20 mg, 0.15 mmol) and TEA (50 mg, 0.5 mmol) in DMF (1 mL) was stirred at r.t. overnight. The reaction mixture was concentrated under reduced pressure. The residue was purified by prep-HPLC on a C18 column eluting with MeCN/H₂O (40-50% with 0.1% TFA) to afford the title compound (8.9 mg) as a white solid. LCMS calc. for C₃₃H₃₂ClF₃N₉O₆ [M−H]⁻: m/z=742.2; Found: 742.1.

Example 367:6-Amino-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-fluoropicolinamide

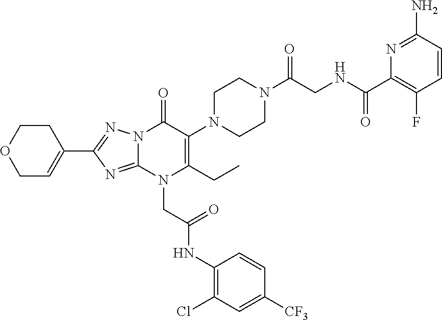 [SMILES: CCc1c(N2CCN(C(=O)CNC(=O)c3nc(N)ccc3F)CC2)c(=O)n2nc(C3=CCOCC3)nc2n1CC(=O)Nc1ccc(C(F)(F)F)cc1Cl]

A mixture of (6-amino-3-fluoropicolinoyl)glycine (30 mg, 0.15 mmol, INT C79), INT B1 (56 mg, 0.1 mmol), EDCI (27 mg, 0.15 mmol), HOBt (20 mg, 0.15 mmol) and TEA (50 mg, 0.5 mmol) in DMF (1 mL) was stirred at r.t. overnight. The mixture was concentrated under reduced pressure. The residue was purified by prep-HPLC on a C18 column eluting with MeCN/H₂O (35-40% with 0.1% TFA) to afford the title compound (1.6 mg) as a white solid. LCMS calc. for C₃₃H₃₄ClF₄N₁₀O₅ [M+H]⁺: m/z=761.2; Found: 761.1.

### Example 368: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-hydroxy-2-methylpyrimidine-4-carboxamide

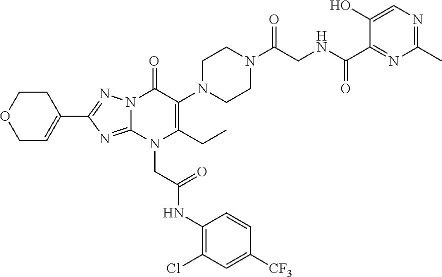 [SMILES: CCc1c(N2CCN(C(=O)CNC(=O)c3nc(C)ncc3O)CC2)c(=O)n2nc(C3=CCOCC3)nc2n1CC(=O)Nc1ccc(C(F)(F)F)cc1Cl]

A mixture of (5-hydroxy-2-methylpyrimidine-4-carbonyl)glycine (35 mg, 0.15 mmol, INT C80), INT B1 (56 mg, 0.1 mmol), EDCI (39 mg, 0.2 mmol), HOBt (27 mg, 0.2 mmol) and TEA (101 mg, 1 mmol) in DMF (1 mL) was stirred at r.t. overnight. The mixture was concentrated under reduced pressure. The residue was purified by flash chromatography on a C18 column eluting with MeCN/H₂O (55-65% with 0.1% TFA) to afford the title compound (32.3 mg) as a white solid. LCMS calc. for C₃₃H₃₃ClF₃N₁₀O₆ [M−H]⁻: m/z=757.2; Found: 757.1.

Example 369: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) isothiazole-5-carboxamide

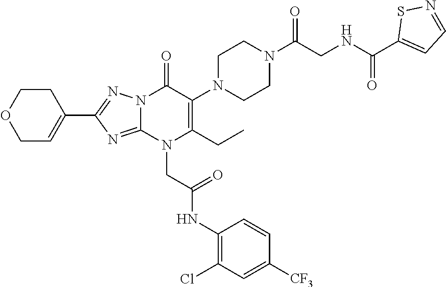 [SMILES: CCc1c(N2CCN(C(=O)CNC(=O)c3ccns3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (100 mg, 0.16 mmol, Example 38) and isothiazole-5-carboxylic acid (31 mg, 0.24 mmol) in DMF (1 mL) was added EDCI (46 mg, 0.24 mmol), HOBt (33 mg, 0.24 mmol) and DIEA (62 mg, 0.48 mmol). The mixture was stirred at r.t. for 3 h., and then concentrated under reduced pressure. The reaction mixture was purified by flash chromatography on a C18 column eluting with MeCN/H₂O (50-60%) to afford the title compound (41.6 mg) as a white solid. LCMS calc. for C₃₁H₃₀ClF₃N₉O₅S [M−H]⁻: m/z=732.2; Found: 732.1.

### Example 370: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) isoxazole-5-carboxamide

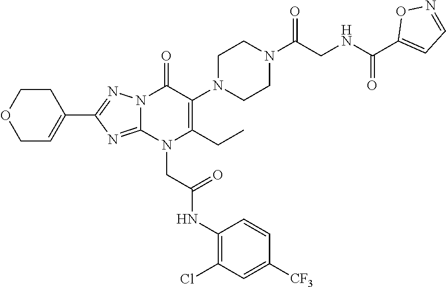 [SMILES: CCc1c(N2CCN(C(=O)CNC(=O)c3ccno3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (100 mg, 0.16 mmol, Example 38) and isoxazole-5-carboxylic acid (27 mg, 0.24 mmol) in DMF (1 mL) was added EDCI (46 mg, 0.24 mmol), HOBt (33 mg, 0.24 mmol) and DIEA (62 mg, 0.48 mmol). The mixture was stirred at r.t. overnight, and then concentrated under reduced pressure. The residue was purified by flash chromatography on a C18 column eluting with MeCN/H₂O (50-60%) to afford the title compound (16.3 mg) as a white solid. LCMS calc. for C₃₁H₃₀ClF₃N₉O₆ [M−H]⁻: m/z=716.2; Found: 716.1.

### Example 371: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl) isoxazole-3-carboxamide

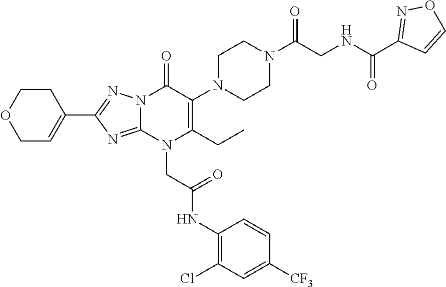 [SMILES: CCc1c(N2CCN(C(=O)CNC(=O)c3ccon3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38) and isoxazole-3-carboxylic acid (5.4 mg, 0.05 mmol) in pyridine (0.3 mL) was added EDCI (15 mg, 0.08 mmol) at r.t. The mixture was stirred at r.t. for 3 h., and then concentrated under reduced pressure. The residue was purified by flash chromatography on a C18 column eluting with MeCN/H₂O (50-60%) to afford the title compound (15.1 mg) as a white solid. LCMS calc. for C₃₁H₃₀ClF₃N₉O₆ [M−H]⁻: m/z=716.2; Found: 716.1.

### Example 372:5-Amino-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluorobenzamide

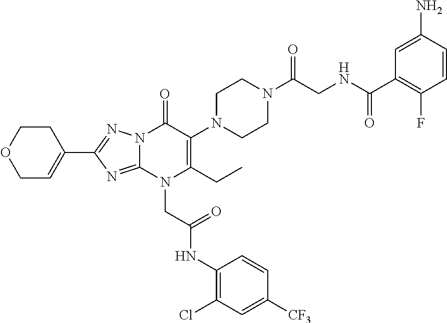 [SMILES: CCc1c(N2CCN(C(=O)CNC(=O)c3cc(N)ccc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (20 mg, 0.03 mmol, Example 38), 5-amino-2-fluorobenzoic acid (5 mg, 0.03 mmol), EDCI (9.2 mg, 0.05 mmol) and HOBt (6.5 mg, 0.05 mmol) in DMF (0.5 mL) was added DIEA (20.7 mg, 0.16 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (11.2 mg) as a white solid. LCMS calc. for C₃₄H₃₃ClF₄N₉O₅ [M−H]⁻: m/z=758.2; Found: 758.1.

### Example 373:5-Amino-N-(2-(4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2,4-difluorobenzamide

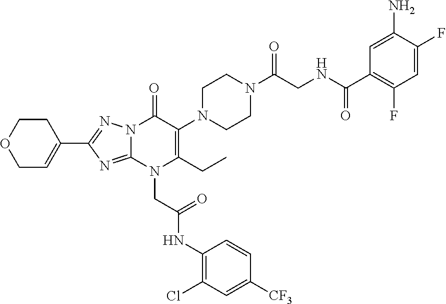 [SMILES: CCc1c(N2CCN(C(=O)CNC(=O)c3cc(N)c(F)cc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (20 mg, 0.03 mmol, Example 38), 5-amino-2,4-difluorobenzoic acid (5.5 mg, 0.03 mmol), EDCI (9.2 mg, 0.05 mmol) and HOBt (6.5 mg, 0.05 mmol) in DMF (0.2 mL) was added DIEA (20.7 mg, 0.16 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (16 mg) as a white solid. LCMS calc. for C₃₄H₃₂ClF₅N₉O₅ [M−H]⁻: m/z=776.2; Found: 776.2.

### Example 374: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-(ethylamino)-2-fluorobenzamide

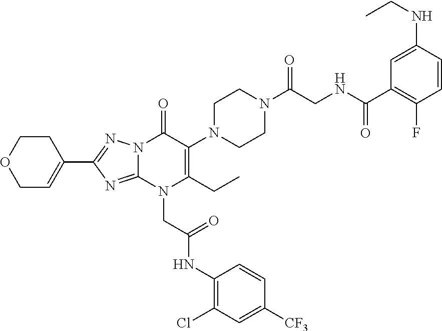 [SMILES: CCNc1ccc(F)c(C(=O)NCC(=O)N2CCN(c3c(CC)n(CC(=O)Nc4ccc(C(F)(F)F)cc4Cl)c4nc(C5=CCOCC5)nn4c3=O)CC2)c1]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.078 mmol, Example 38) and 5-(ethylamino)-2-fluorobenzoic acid (21 mg, 0.117 mmol) in DMF (0.5 mL) was added EDCI (22 mg, 0.08 mmol), HOBt (16 mg, 0.117 mmol) and DIEA (30 mg, 0.235 mmol) at r.t. The mixture was stirred at r.t. for 3 h., and then concentrated under reduced pressure. The reaction mixture was purified by flash chromatography on a C18 column eluting with MeCN/H₂O (30-40%) to afford the title compound (25.4 mg) as a white solid. LCMS calc. for C₃₆H₃₇ClF₄N₉O₅ [M−H]⁻: m/z=786.3; Found: 786.1.

### Example 375: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-fluorothiophene-2-carboxamide

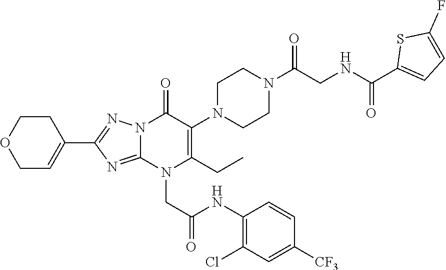 [SMILES: CCc1c(N2CCN(C(=O)CNC(=O)c3ccc(F)s3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.08 mmol, Example 38) and 5-fluorothiophene-2-carboxylic acid (11.7 mg, 0.08 mmol) in pyridine (0.5 mL) was added EDCI (23.1 mg, 0.12 mmol) at r.t. The mixture was stirred at r.t. for 3 h., and then concentrated under reduced pressure. The residue was purified by flash chromatography on a C18 column eluting with MeCN/H₂O (40-60%) to afford the title compound (22.4 mg) as a white solid. LCMS calc. for C₃₂H₃₀ClF₄N₈O₅S [M−H]⁻: m/z=749.2; Found: 749.1.

### Example 376: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl) isoxazole-5-carboxamide

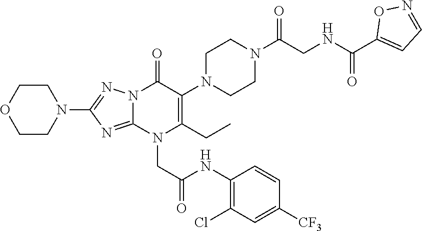 [SMILES: CCc1c(N2CCN(C(=O)CNC(=O)c3ccno3)CC2)c(=O)n2nc(N3CCOCC3)nc2n1CC(=O)Nc1ccc(C(F)(F)F)cc1Cl]

To a mixture of N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide (40 mg, 0.06 mmol, Example 248 step 2), isoxazole-5-carboxylic acid (7.2 mg, 0.06 mmol), EDCI (16.6 mg, 0.09 mmol) and HOBt (12 mg, 0.09 mmol) in DMF (0.5 mL) was added DIEA (38.7 mg, 0.3 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (12.2 mg) as a white solid. LCMS calc. for C₃₀H₃₁ClF₃N₁₀O₆ [M−H]⁻: m/z=719.2; Found: 719.2.

### Example 377: N-(2-(4-(4-(2-((2-Chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin-1-yl)-2-oxoethyl)-5-hydroxy-6-methylpyrimidine-4-carboxamide

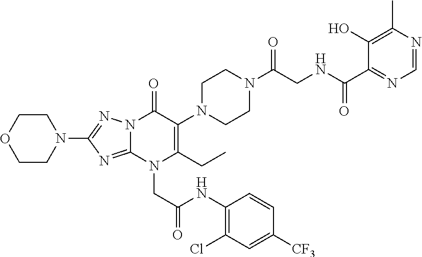 [SMILES: CCc1c(N2CCN(C(=O)CNC(=O)c3ncnc(C)c3O)CC2)c(=O)n2nc(N3CCOCC3)nc2n1CC(=O)Nc1ccc(C(F)(F)F)cc1Cl]

To a mixture of N-(2-chloro-4-(trifluoromethyl)phenyl)-2-(5-ethyl-6-(4-glycylpiperazin-1-yl)-2-morpholino-7-oxo-[1,2,4]triazolo[1,5-a]pyrimidin-4(7H)-yl)acetamide (40 mg, 0.06 mmol, Example 248 step 2), 5-hydroxy-6-methylpyrimidine-4-carboxylic acid (INT C81, 10 mg, 0.06 mmol) in pyridine (0.5 mL) was added EDCI (17.8 mg, 0.09 mmol) at r.t. The mixture was stirred at r.t. for 3 h., and then concentrated under reduced pressure. The residue was purified by flash chromatography on a C18 column eluting with MeCN/H₂O (45-60%) to afford the title compound (3.5 mg) as a white solid. LCMS calc. for C₃₂H₃₄ClF₃N₁₁O₆ [M−H]⁻: m/z=760.2; Found: 760.2.

Example 378: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-5-methoxybenzamide

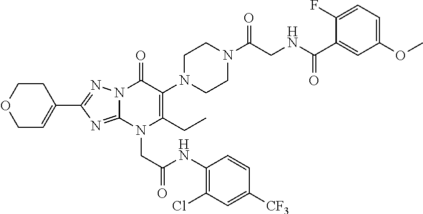 [SMILES: CCc1c(N2CCN(C(=O)CNC(=O)c3cc(OC)ccc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 2-fluoro-5-methoxybenzoic acid (8.5 mg, 0.05 mmol), EDCI (15 mg, 0.08 mmol) and DIEA (19.1 mg, 0.15 mmol) in DMF (0.3 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (17.1 mg) as a white solid. LCMS calc. for C₃₅H₃₄ClF₄N₈O₆ [M−H]⁻: m/z=773.2; Found: 773.1.

### Example 379: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-4-methoxybenzamide

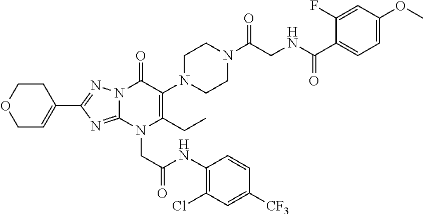 [SMILES: CCc1c(N2CCN(C(=O)CNC(=O)c3ccc(OC)cc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 2-fluoro-4-methoxybenzoic acid (8.5 mg, 0.05 mmol), EDCI (15 mg, 0.08 mmol) and DIEA (19.1 mg, 0.15 mmol) in DMF (0.3 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (17.7 mg) as a white solid. LCMS calc. for C₃₅H₃₄ClF₄N₈O₆ [M−H]⁻: m/z=773.2; Found: 773.1.

### Example 380: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-2-fluoro-4-(methylamino)benzamide

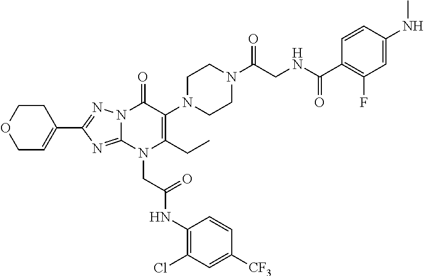 [SMILES: CCc1c(N2CCN(C(=O)CNC(=O)c3ccc(NC)cc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 2-fluoro-4-(methylamino)benzoic acid (8.2 mg, 0.05 mmol), EDCI (15 mg, 0.08 mmol) and DIEA (19.1 mg, 0.15 mmol) in DMF (0.3 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (45-60%) to afford the title compound (18.5 mg) as a white solid. LCMS calc. for C₃₅H₃₅ClF₄N₉O₅ [M−H]⁻: m/z=772.3; Found: 772.2.

### Example 381: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-3-hydroxyisonicotinamide

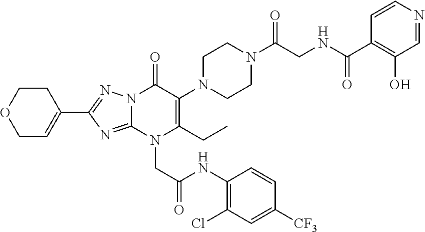 [SMILES: CCc1c(N2CCN(C(=O)CNC(=O)c3ccncc3O)CC2)c(=O)n2nc(C3=CCOCC3)nc2n1CC(=O)Nc1ccc(C(F)(F)F)cc1Cl]

To a mixture of N-(2-Chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (40 mg, 0.06 mmol, Example 38) and 3-hydroxyisonicotinic acid (8.9 mg, 0.06 mmol) in pyridine (0.5 mL) was added EDCI (17.8 mg, 0.09 mmol) at r.t. The mixture was stirred at r.t. for 3 h., and then concentrated under reduced pressure. The reaction mixture was purified by flash chromatography on a C18 column eluting with MeCN/H₂O (40-60%) to afford the title compound (3.5 mg) as a white solid. LCMS calc. for C₃₃H₃₂ClF₃N₉O₆ [M−H]⁻: m/z=742.2; Found: 742.2.

### Example 382: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-5-cyano-2-fluorobenzamide

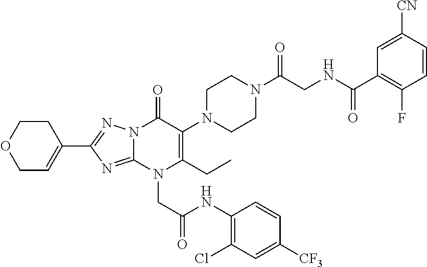 [SMILES: CCc1c(N2CCN(C(=O)CNC(=O)c3cc(C#N)ccc3F)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 5-cyano-2-fluorobenzoic acid (8.0 mg, 0.05 mmol), EDCI (15 mg, 0.08 mmol) and DIEA (19.1 mg, 0.15 mmol) in DMF (0.5 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (40-60%) to afford the title compound (14.3 mg) as a white solid. LCMS calc. for C₃₅H₃₁ClF₄N₉O₅ [M−H]⁻: m/z=768.2; Found: 768.1.

### Example 383: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)benzo[c][1,2,5]oxadiazole-4-carboxamide

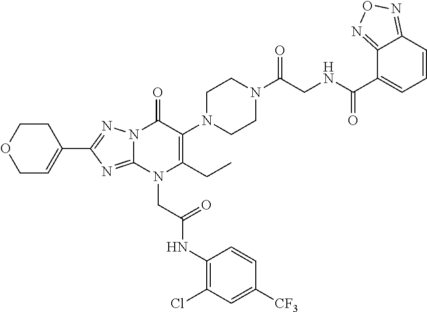 [SMILES: CCc1c(N2CCN(C(=O)CNC(=O)c3cccc4nonc34)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), benzo[c][1,2,5]oxadiazole-4-carboxylic acid (8.2 mg, 0.05 mmol), EDCI (15 mg, 0.08 mmol) and DIEA (19.1 mg, 0.15 mmol) in DMF (0.3 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (40-60%) to afford the title compound (25.7 mg) as a white solid. LCMS calc. for C₃₄H₃₁ClF₃N₁₀O₆ [M−H]⁻: m/z=767.2; Found: 767.1.

### Example 384: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)-1,2,5-oxadiazole-3-carboxamide

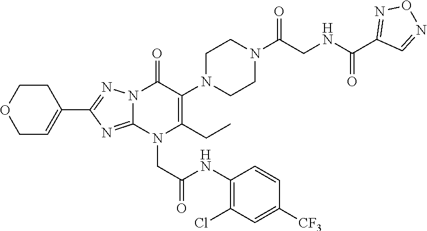 [SMILES: CCc1c(N2CCN(C(=O)CNC(=O)c3cnon3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (30 mg, 0.05 mmol, Example 38), 1,2,5-oxadiazole-3-carboxylic acid (5.5 mg, 0.05 mmol), EDCI (15 mg, 0.08 mmol) and DIEA (19.1 mg, 0.15 mmol) in DMF (0.3 mL) was added HOBt (10.8 mg, 0.08 mmol) at 0° C. The mixture was stirred at r.t. for 3 h., and then concentrated under reduced pressure. The residue was purified by flash chromatography on a C18 column eluting with MeCN/H₂O (40-60%) to afford the title compound (2.7 mg) as a white solid. LCMS calc. for C₃₀H₂₉ClF₃N₁₀O₆ [M−H]⁻: m/z=717.2; Found: 717.2.

### Example 385: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)furan-2-carboxamide

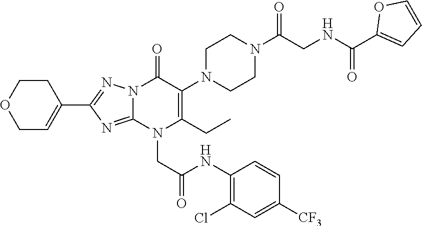 [SMILES: CCc1c(N2CCN(C(=O)CNC(=O)c3ccco3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.08 mmol, Example 38) and furan-2-carboxylic acid (9.0 mg, 0.08 mmol) in pyridine (0.5 mL) was added EDCI (23.1 mg, 0.12 mmol) at r.t. The mixture was stirred at r.t. for 3 h., and then concentrated under reduced pressure. The residue was purified by flash chromatography on a C18 column eluting with MeCN/H₂O (40-60%) to afford the title compound (42.6 mg) as a white solid. LCMS calc. for C₃₂H₃₁ClF₃N₈O₆ [M−H]⁻: m/z=715.2; Found: 715.1.

### Example 386: N-(2-(4-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazin-1-yl)-2-oxoethyl)thiophene-2-carboxamide

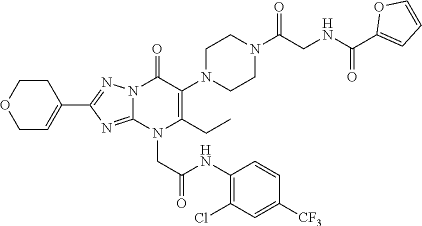 [SMILES: CCc1c(N2CCN(C(=O)CNC(=O)c3ccco3)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-6-(4-glycylpiperazin-1-yl)-7-oxo-[1,2,4]triazolo[1,5-a]pyrimidin-4(7H)-yl)acetamide (50 mg, 0.08 mmol, Example 38) and thiophene-2-carboxylic acid (10.3 mg, 0.08 mmol) in pyridine (0.5 mL) was added EDCI (23.1 mg, 0.12 mmol) at r.t. The mixture was stirred at r.t. for 3 h., and then concentrated under reduced pressure. The residue was purified by flash chromatography on a C18 column eluting with MeCN/H₂O (40-60%) to afford the title compound (26.5 mg) as a white solid. LCMS calc. for C₃₂H₃₁ClF₃N₈O₅S [M−H]⁻: m/z=731.2; Found: 731.1.

### Intermediates

### INT A1:2-Chloro-N-(2-chloro-4-(trifluoromethyl)phenyl)acetamide

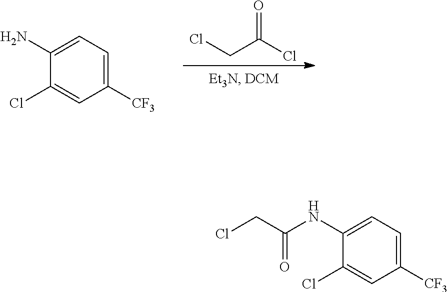 [SMILES: Nc1ccc(C(F)(F)F)cc1Cl ; O=C(Cl)CCl ; O=C(CCl)Nc1ccc(C(F)(F)F)cc1Cl]

A mixture of 2-chloro-4-(trifluoromethyl) aniline (15 g, 76.9 mmol), 2-chloroacetyl chloride (13 g, 115 mmol) and triethylamine (23.3 g, 230.8 mmol) in DCM (150 mL) was stirred at r.t. overnight. The reaction mixture was diluted with water (200 mL), extracted with DCM (200 mL×3). The combined organic layers were dried over anhydrous Na₂SO₄, filtered and concentrated under reduced pressure. The residue was purified by flash chromatography on a silica gel column eluting with DCM/PE (0-40%) to afford the title compound (12.3 g, 59% yield). ¹H NMR (400 MHZ, CDCl₃) δ ppm 9.08 (s, 1H), 8.58 (d, J=8.7 Hz, 1H), 7.69 (d, J=1.6 Hz, 1H), 7.57 (dd, J=8.7, 1.3 Hz, 1H), 4.26 (s, 2H).

### INT A2: 2-Chloro-N-(2-methyl-4-(trifluoromethyl)phenyl)acetamide

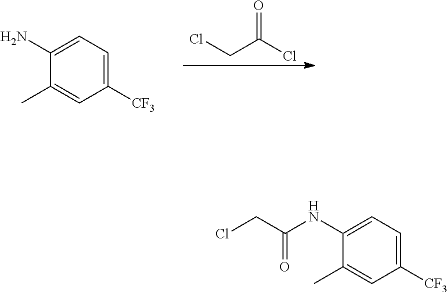 [SMILES: Cc1cc(C(F)(F)F)ccc1N ; O=C(Cl)CCl ; Cc1cc(C(F)(F)F)ccc1NC(=O)CCl]

This compound was prepared as a white solid by procedures analogous to those described for INT A1 using 2-methyl-4-(trifluoromethyl) aniline to replace 2-chloro-4-(trifluoromethyl) aniline to afford the title product. ¹H NMR (400 MHZ, CDCl₃) δ ppm 8.37 (s, 1H), 8.18 (d, J=8.4 Hz, 1H), 7.51 (d, J=8.5 Hz, 1H), 7.47 (s, 1H), 4.26 (s, 2H), 2.37 (s, 3H).

### INT A3: N-(3-Bromo-5-methylthiophen-2-yl)-2-chloroacetamide

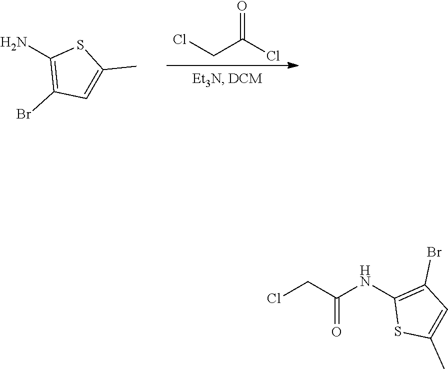

This compound was prepared by procedures analogous to those described for INT A1 using 3-bromo-5-methylthiophen-2-amine to replace 2-chloro-4-(trifluoromethyl) aniline to afford the title product as a white solid. LCMS calc. for C7H₈BrClNOS [M+H]⁺: m/z=267.9; Found: 268.5, 270.5.

### INT A4: 2-Chloro-N-(3,5-dimethylthiophen-2-yl)acetamide

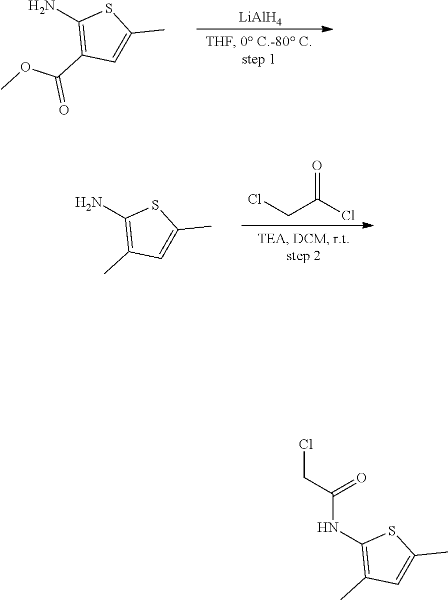

### Step 1: 3,5-dimethylthiophen-2-amine

To a solution of methyl 2-amino-5-methyl-thiophene-3-carboxylate (500 mg, 5.85 mmol) in THF (30 mL) was added LiAlH₄ (667 mg, 17.54 mmol) at 0° C. The mixture was stirred at 80° C. for 3 h. The reaction was quenched with ice-water (0.7 mL), stirred for 20 min. and then treated with 10% NaOH aq. (0.7 mL). The mixture was filtered through Celite and the filtrate was concentrated under reduced pressure to give the crude product (233 mg, 31% yield) as a yellow liquid which was used directly without purification. LCMS calc. for C₆H₁₀NS [M+H]⁺: m/z=128.0; Found: 128.0.

### Step 2: 2-chloro-N-(3,5-dimethylthiophen-2-yl)acetamide

This compound was prepared by procedures analogous to those described for INT A1 using 3-methyl-5-methylthiophen-2-amine to replace 2-chloro-4-(trifluoromethyl) aniline to afford the title product as a brown solid. LCMS calc. for C₈H₁₁ClNOS [M+H]⁺: m/z=204.0; Found: 204.0.

### INT B1: N-(2-Chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

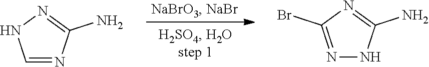 [SMILES: Nc1nc[nH]n1 ; Nc1nc(Br)n[nH]1]

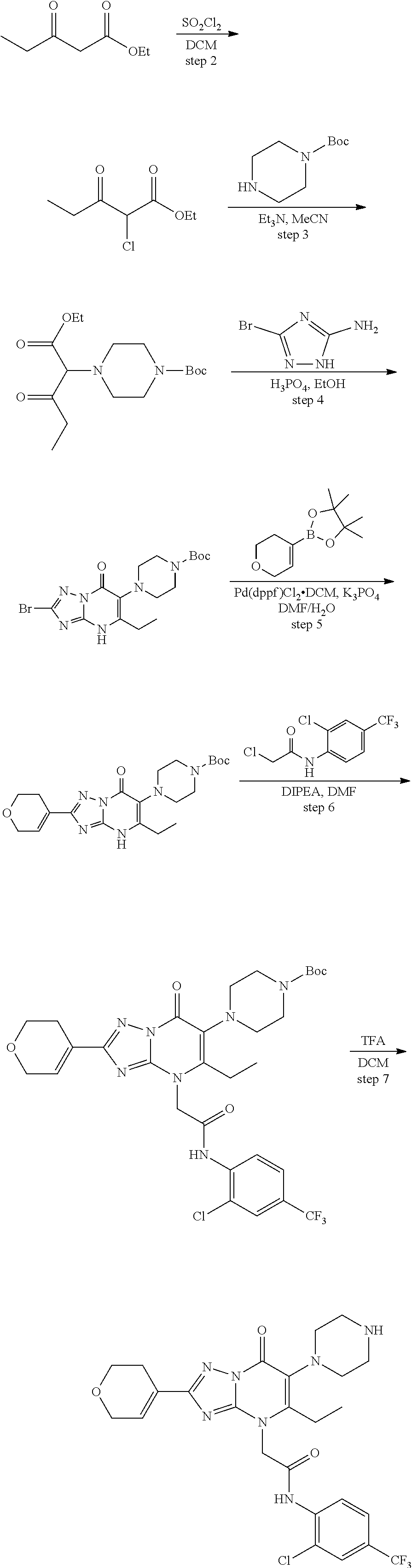

### Step 1: 3-bromo-1H-1,2,4-triazol-5-amine

Water (300 mL), sulfuric acid (150 g), 3-amino-1,2,4-triazole (50 g) and sodium bromide (150 g) were added to the reactor, and the system was heated to 55° C.-60° C. Sodium bromate (45 g) in water (150 mL) was added dropwise to the reactor at 55° C.-60° C. The mixture was stirred for 20 h., cooled to 20° C.-30° C., quenched with saturated NaHSO₃ aq. The resulting mixture was adjusted to PH˜5.5-6.5 with 20% sodium hydroxide solution, and then filtered. n-Butanol was added to the filtrate, and concentrated at 55° C.-60° C. The residue was dissolved in ethyl acetate, filtered through a pad of silica gel. The filtrate was concentrated. The residue was dissolved in 1,4-dioxane at 80° C., and cooled to 20° C.-30° C. The solid formed was collected by filtration and dried at 50° C.-60° C. in vacuum overnight to afford the title compound (48.8 g, 50% yield). ¹H NMR (400 MHZ, DMSO-d₆) δ ppm 12.27 (s, 1H), 6.30 (s, 2H).

### Step 2: ethyl 2-chloro-3-oxopentanoate

To a solution of ethyl 3-oxopentanoate (10 g, 69.4 mmol) in DCM (60 mL) was added SO₂Cl₂ (12.2 g, 90.2 mmol) at r.t. over 5 min., and stirred at r.t. overnight. The reaction mixture was concentrated under reduced pressure. The residue was dissolved in DCM (60 mL) and washed with water and brine, dried over anhydrous Na₂SO₄ and filtered. The filtrate was concentrated under reduced pressure. The residue was purified by flash chromatography on a silica gel column eluting with EtOAc/PE (5%-10%) to give the title compound (10 g, 81% yield) as a light-yellow liquid. ¹H NMR (400 MHZ, CDCl₃) δ ppm 12.42 (s, 0.3H), 4.80 (s, 0.6H), 4.34-4.25 (m, 2H), 2.82-2.66 (m, 1H), 2.61-2.48 (m, 1H), 1.40-1.29 (m, 3H), 1.21-1.09 (m, 3H).

### Step 3: tert-butyl 4-(1-ethoxy-1,3-dioxopentan-2-yl)piperazine-1-carboxylate

To a solution of ethyl 2-chloro-3-oxopentanoate (10 g, 56 mmol) in dry MeCN (56 mL) was added dropwise Et₃N (23.3 mL, 168 mmol) for about 1.5 h., then was added dropwise tert-butyl piperazine-1-carboxylate (10.43 g, 56 mmol) in MeCN (52 mL) for about 2.5 h. After stirring at 60° C. overnight, the reaction mixture was filtered and washed with EtOAc. The filtrate was concentrated under reduced pressure. The residue was dissolved in EtOAc, washed with water, dried over anhydrous Na₂SO₄ and filtered. The filtrate was concentrated under reduced pressure. The residue was purified by flash chromatography on a silica gel column eluting with EtOAc/PE (0-25%) to give the title compound (13.2 g, 72% yield) as a yellow liquid. ¹H NMR (400 MHZ, CDCl₃) δ ppm 12.20 (s, 0.18H), 4.31-4.19 (m, 2H), 3.95 (s, 0.49H), 3.53 (s, 3H), 3.07 (t, J=10.8, 1H), 3.00-2.43 (m, 6H), 1.50-1.43 (m, 9H), 1.36-1.27 (m, 3H), 1.15-1.06 (m, 3H). LCMS calc. for C₁₆H₂₈N₂O₅ [M+H]⁺: m/z=329.2; Found: 329.3.

### Step 4: tert-butyl 4-(2-bromo-5-ethyl-7-oxo-4,7-dihydro-[1,2,4]triazolo[1,5-a]pyrimidin-6-yl)piperazine-1-carboxylate

A mixture of 3-bromo-1H-1,2,4-triazol-5-amine (26.9 g, 166 mmol), tert-butyl 4-(1-ethoxy-1,3-dioxopentan-2-yl)piperazine-1-carboxylate (60.0 g, 183 mmol) and H₃PO₄ (16.3 g, 166 mmol) in EtOH (160 mL) was stirred at 80° C. overnight under nitrogen. The mixture was concentrated in vacuo to remove EtOH, then quenched with sat aq. NaHCO₃ (300 mL), and extracted with DCM (300 mL×3). The combined organic layers were washed with brine (300 mL×3), dried over anhydrous Na₂SO₄, filtered and concentrated under reduced pressure. The crude product was purified by flash chromatography on a silica gel column eluting with MeOH/DCM (0-3%) to afford the title compound (14.2 g, 20% yield). ¹H NMR (400 MHZ, DMSO-d₆) δ ppm 13.25 (s, 1H), 3.90 (s, 2H), 3.29 (s, 2H), 2.88 (s, 2H), 2.76 (q, J=7.5 Hz, 2H), 2.62 (d, J=10.2 Hz, 2H), 1.47-1.39 (m, 9H), 1.18 (t, J=7.5 Hz, 3H).

### Step 5: tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

A mixture of tert-butyl 4-(2-bromo-5-ethyl-7-oxo-4,7-dihydro-[1,2,4]triazolo[1,5-a]pyrimidin-6-yl)piperazine-1-carboxylate (20.2 g, 47.1 mmol), 2-(3,6-dihydro-2H-pyran-4-yl)-4,4,5,5-tetramethyl-1,3,2-dioxaborolane (19.8 g, 94.3 mmol), Pd(dppf)Cl₂·DCM (1.9 g, 2.4 mmol) and K₃PO₄ (30.0 g, 141.3 mmol) in DMF/H₂O (v:v=7:3) (200 mL) was degassed and recharged with argon for three cycles. The mixture was stirred at 100° C. overnight. The mixture was extracted with DCM. The organic layer was washed with brine, dried over Na₂SO₄ and concentrated under reduced pressure. The residue was purified by flash chromatography on a silica gel column eluting with MeOH/DCM (0-2%) to afford the title compound (11.3 g, 55% yield). LCMS calc. for C₂₁H₃₁N₆O₄ [M+H]⁺: m/z=431.3; Found: 431.3.

### Step 6: tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

A mixture of tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (11.3 g, 26.3 mmol), INT A1 (6.8 g, 25.0 mmol) and DIPEA (10.2 g, 78.9 mmol) in DMF (110 mL) was stirred at 80° C. overnight. The reaction mixture was concentrated, extracted with 10% MeOH in DCM, dried over anhydrous Na₂SO₄ and concentrated under reduced pressure. The residue was purified by flash chromatography on a silica gel column eluting with MeOH/DCM (0-2%) to afford the title compound (12 g, 69% yield). LCMS calc. for C₃₀H₃₆ClF₃N₇O₅ [M+H]⁺: m/z=666.2; Found: 666.2.

### Step 7: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

tert-Butyl 4-(2-(3,6-dihydro-2H-pyran-4-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)piperazine-1-carboxylate (12 g, 18.0 mmol) suspended in DCM (90 mL) was treated with TFA (30 mL) at 0° C. The mixture was stirred at r.t. for 2 h., quenched with aq. NaHCO₃, and extracted with DCM. The organic layer was dried over anhydrous Na₂SO₄ and concentrated under reduced pressure. The crude product was purified by flash chromatography on a C18 column eluting with MeOH/H₂O (0-60%, with 0.5% NH₄HCO₃) to afford the title compound (8.3 g, 81% yield). ¹H NMR (400 MHZ, DMSO-d₆) δ ppm 10.38 (s, 1H), 8.07 (d, J=8.4 Hz, 1H), 7.96 (d, J=1.6 Hz, 1H), 7.71 (dd, J=8.7, 1.7 Hz, 1H), 6.82 (s, 1H), 5.30 (s, 2H), 4.25 (d, J=2.6 Hz, 2H), 3.81 (t, J=5.4 Hz, 2H), 3.47 (dd, J=11.2, 9.1 Hz, 2H), 2.95 (q, J=7.3 Hz, 2H), 2.88 (d, J=11.5 Hz, 2H), 2.72 (t, J=10.4 Hz, 2H), 2.54 (d, J=11.5 Hz, 4H), 1.17 (t, J=7.4 Hz, 3H). LCMS calc. for C₂₅H₂₈ClF₃N₇O₃ [M+H]⁺: m/z=566.2; Found: 566.2.

### INT B2:2-(2-(3,6-Dihydro-2H-pyran-4-yl)-5-ethyl-7-oxo-6-(piperazin-1-yl)-[1,2,4]triazolo[1,5-a]pyrimidin-4(7H)-yl)-N-(2-methyl-4-(trifluoromethyl)phenyl)acetamide

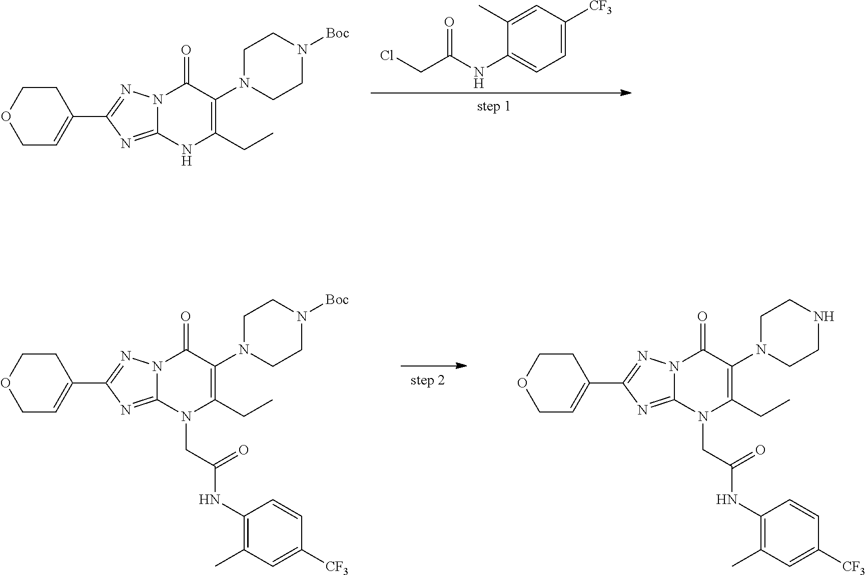 [SMILES: CCc1[nH]c2nc(C3=CCOCC3)nn2c(=O)c1N1CCN(C(=O)OC(C)(C)C)CC1 ; Cc1cc(C(F)(F)F)ccc1NC(=O)CCl ; CCc1c(N2CCN(C(=O)OC(C)(C)C)CC2)c(=O)n2nc(C3=CCOCC3)nc2n1CC(=O)Nc1ccc(C(F)(F)F)cc1C ; CCc1c(N2CCNCC2)c(=O)n2nc(C3=CCOCC3)nc2n1CC(=O)Nc1ccc(C(F)(F)F)cc1C]

This compound was prepared by procedures analogous to those described for INT B1 Step 6-7 using INT A2 to replace INT A1 in Step 6 to afford the title product as a white solid. LCMS calc. for C₂₆H₃₁F₃N₇O₃ [M+H]⁺: m/z=546.2; Found: 546.3.

### INT B3: N-(2-Chloro-4-(trifluoromethyl)phenyl)-2-(2-(3,6-dihydro-2H-pyran-4-yl)-5-ethyl-7-oxo-6-(piperazin-1-yl)pyrazolo[1,5-a]pyrimidin-4(7H)-yl)acetamide

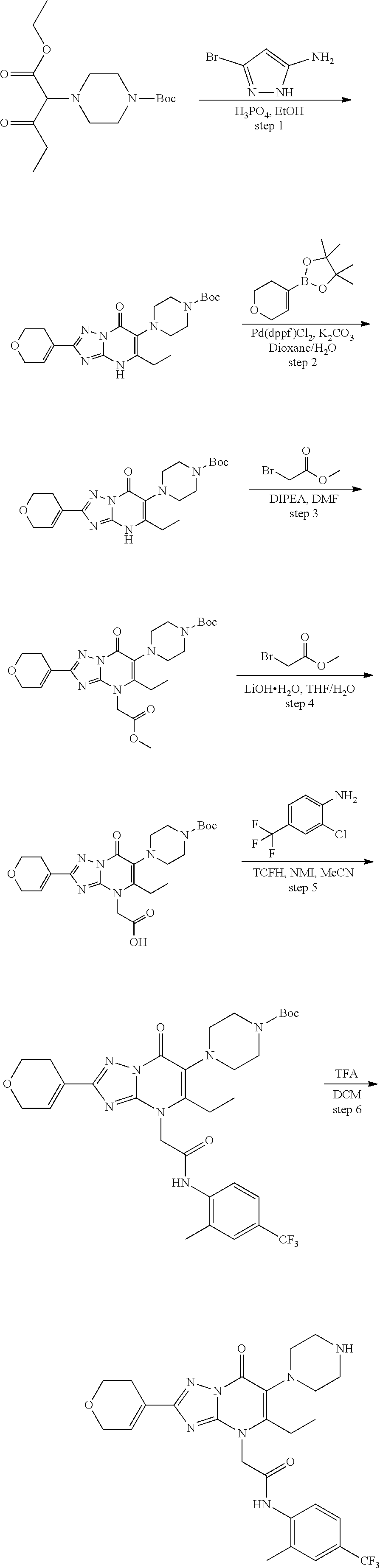

### Step 1: tert-butyl 4-(2-bromo-5-ethyl-7-oxo-4,7-dihydropyrazolo[1,5-a]pyrimidin-6-yl)piperazine-1-carboxylate

To a mixture of 3-bromo-1H-pyrazol-5-amine (100 mg, 0.62 mmol) in EtOH (2 mL) and H₃PO₄ (60 mg, 0.62 mmol) was added tert-butyl 4-(1-ethoxy-1,3-dioxopentan-2-yl)piperazine-1-carboxylate (304 mg, 0.93 mmol, INT B1 Step 3). The reaction mixture was degassed and recharged with nitrogen for 3 cycles. The mixture was stirred at 80° C. for 8 h., cooled to 0° C., quenched with H₂O (10 mL). The precipitate was collected by filtration, washed with water (5 mL) and dried under reduced pressure to afford the title compound (250 mg, 95% yield) as a white solid. LCMS calc. for C₁₇H₂₃BrN₅O₃ [M−H]⁻: m/z=424.1; Found: 423.9.

### Step 2: tert-butyl 4-(2-(3,6-dihydro-2H-pyran-4-yl)-5-ethyl-7-oxo-4,7-dihydropyrazolo[1,5-a]pyrimidin-6-yl)piperazine-1-carboxylate

A mixture of tert-butyl 4-(2-bromo-5-ethyl-7-oxo-4,7-dihydropyrazolo[1,5-a]pyrimidin-6-yl)piperazine-1-carboxylate (250 mg, 0.59 mmol), 2-(3,6-dihydro-2H-pyran-4-yl)-4,4,5,5-tetramethyl-1,3,2-dioxaborolane (246 mg, 1.17 mmol), Pd(dppf)Cl₂ (43 mg, 0.06 mmol), and K₂CO₃ (243 mg, 1.76 mmol) in dioxane (2 mL) and H₂O (0.4 mL) was degassed and recharged with nitrogen for 3 cycles, and then stirred at 80° C. for 8 h. The resulting mixture was diluted with H₂O (10 mL) and extracted with EtOAc (10 mL×3). The combined organic layers were washed with brine (30 mL), dried over Na₂SO₄, filtered and concentrated under reduced pressure. The residue was purified by flash chromatography on a silica gel column eluting with MeOH/CH₂Cl₂ (5-8%) to afford the title compound (200 mg, 79% yield) as a brown solid. LCMS calc. for C₂₂H₃₀N₅O₄ [M−H]⁻: m/z=428.2; Found: 428.2.

### Step 3: tert-butyl 4-(2-(3,6-dihydro-2H-pyran-4-yl)-5-ethyl-4-(2-methoxy-2-oxoethyl)-7-oxo-4,7-dihydropyrazolo[1,5-a]pyrimidin-6-yl)piperazine-1-carboxylate

To mixture of a tert-butyl 4-(2-(3,6-dihydro-2H-pyran-4-yl)-5-ethyl-7-oxo-4,7-dihydropyrazolo[1,5-a]pyrimidin-6-yl)piperazine-1-carboxylate (150 mg, 0.35 mmol) and DIPEA (135 mg, 1.05 mmol) in DMF (2 mL) was added methyl 2-bromoacetate (107 mg, 0.7 mmol) at 0° C. The mixture was stirred at r.t. for 2 h., diluted with H₂O (10 mL) and extracted with EtOAc (10 mL×3). The combined organic layers were washed with brine (20 mL), dried over Na₂SO₄, filtered and concentrated under reduced pressure. The residue was purified by flash chromatography on a silica gel column eluting with EA/PE (50%-80%) to afford the title compound (150 mg, 86% yield) as a brown solid. LCMS calc. for C₂₅H₃₆N₅O₆ [M+H]⁺: m/z=502.3; Found: 502.3.

### Step 4: 2-(6-(4-(tert-butoxycarbonyl)piperazin-1-yl)-2-(3,6-dihydro-2H-pyran-4-yl)-5-ethyl-7-oxopyrazolo[1,5-a]pyrimidin-4(7H)-yl)acetic acid

A mixture of tert-butyl 4-(2-(3,6-dihydro-2H-pyran-4-yl)-5-ethyl-4-(2-methoxy-2-oxoethyl)-7-oxo-4,7-dihydropyrazolo[1,5-a]pyrimidin-6-yl)piperazine-1-carboxylate (150 mg, 0.3 mmol) and LiOH·H₂O (25 mg, 0.6 mmol) in THF (2 mL) and H₂O (1 mL) was stirred at 25° C. for 2 h. The resulting mixture was diluted with H₂O (10 mL), adjusted to pH=3-4 with HCl aq. (2 N), and concentrate under reduced pressure. The residue was purified by flash chromatography on a C18 column eluting with MeCN/H₂O (30-50%) to afford the title compound (100 mg, 68% yield) as a brown solid. LCMS calc. for C₂₄H₃₄N₅O₆ [M+H]⁺: m/z=488.2; Found: 488.3.

### Step 5: tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydropyrazolo[1,5-a]pyrimidin-6-yl)piperazine-1-carboxylate

To a mixture of 2-(6-(4-(tert-butoxycarbonyl)piperazin-1-yl)-2-(3,6-dihydro-2H-pyran-4-yl)-5-ethyl-7-oxopyrazolo[1,5-a]pyrimidin-4(7H)-yl)acetic acid (100 mg, 0.21 mmol), 2-chloro-4-(trifluoromethyl) aniline (80 mg, 0.41 mmol) and NMI (51 mg, 0.62 mmol) in MeCN (1 mL) was added TCFH (86 mg, 0.31 mmol). The mixture was stirred at r.t. overnight and then concentrated under reduced pressure. The residue was purified by flash chromatography on a C18 column eluting with MeCN/H₂O (70%-90%) to afford the title compound (100 mg, 72% yield) as a brown solid. LCMS calc. for C₃₁H₃₅ClF₃N₆O₅ [M−H]⁻: m/z=663.2; Found: 663.2.

### Step 6: N-(2-chloro-4-(trifluoromethyl)phenyl)-2-(2-(3,6-dihydro-2H-pyran-4-yl)-5-ethyl-7-oxo-6-(piperazin-1-yl)pyrazolo[1,5-a]pyrimidin-4(7H)-yl)acetamide

To a solution of tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydropyrazolo[1,5-a]pyrimidin-6-yl)piperazine-1-carboxylate (100 mg, 0.15 mmol) in DCM (1 mL) was added TFA (1 mL). The mixture was stirred at r.t. for 2 h., and concentrated under reduced pressure to afford the title compound (80 mg, crude) as TFA salt as a brown solid which was used directly in next step. LCMS calc. for C₂₆H₂₉ClF₃N₆O₃ [M+H]⁺: m/z=565.2; Found: 565.3.

### INT B4: N-(2-Chloro-4-(trifluoromethyl)phenyl)-2-(5-ethyl-2-morpholino-7-oxo-6-(piperazin-1-yl)-[1,2,4]triazolo[1,5-a]pyrimidin-4(7H)-yl)acetamide

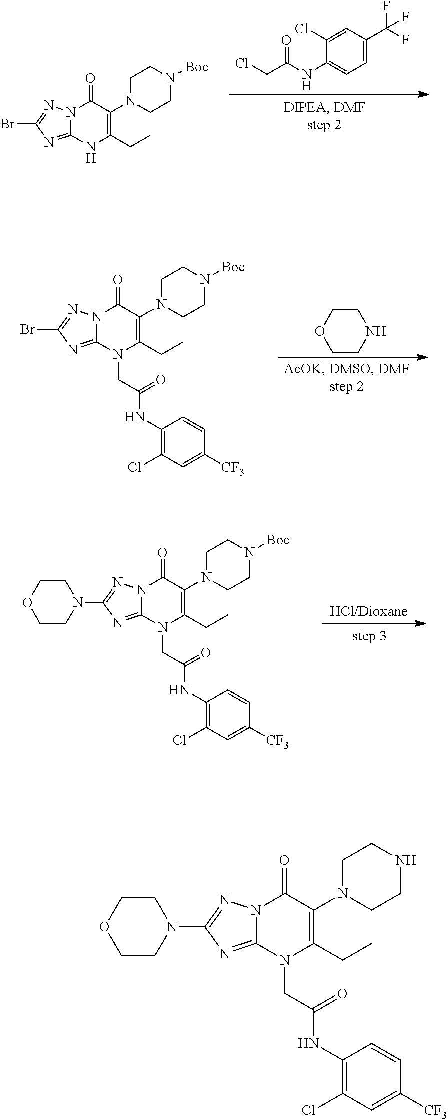 [SMILES: CCc1[nH]c2nc(Br)nn2c(=O)c1N1CCN(C(=O)OC(C)(C)C)CC1 ; O=C(CCl)Nc1ccc(C(F)(F)F)cc1Cl ; CCc1c(N2CCN(C(=O)OC(C)(C)C)CC2)c(=O)n2nc(Br)nc2n1CC(=O)Nc1ccc(C(F)(F)F)cc1Cl ; C1COCCN1 ; CCc1c(N2CCN(C(=O)OC(C)(C)C)CC2)c(=O)n2nc(N3CCOCC3)nc2n1CC(=O)Nc1ccc(C(F)(F)F)cc1Cl ; CCc1c(N2CCNCC2)c(=O)n2nc(N3CCOCC3)nc2n1CC(=O)Nc1ccc(C(F)(F)F)cc1Cl]

### Step 1: tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

To a solution of tert-butyl 4-(2-bromo-5-ethyl-7-oxo-4,7-dihydro-[1,2,4]triazolo[1,5-a]pyrimidin-6-yl)piperazine-1-carboxylate (3 g, 7.02 mmol, INT B1 Step 4) and INT A1 (2.3 g, 8.42 mmol) in DMF (70 mL) was added DIPEA (2.9 g, 22.46 mmol). The mixture was stirred at 80° C. for 16 h. and cooled to r.t., and then concentrated. The residue was diluted with EtOAc (300 mL), washed with sat. NH₄Cl aq. (100 mL×3). The combined organic layers were dried over Na₂SO₄, filtered and concentrated. The residue was purified by flash chromatography on a silica gel column eluting with EtOAc/PE (21%) to afford the title compound (1.75 g, 37% yield) as a yellow solid. ¹H NMR (400 MHz, DMSO-d₆) δ ppm 10.33 (s, 1H), 8.08 (d, J=8.6 Hz, 1H), 7.97 (d, J=1.6 Hz, 1H), 7.74-7.71 (m, 1H), 5.30 (s, 2H), 3.94 (d, J=12.6 Hz, 2H), 3.40-3.33 (m, 2H), 2.97 (d, J=7.3 Hz, 4H), 2.66 (d, J=11.5 Hz, 2H), 1.43 (s, 9H), 1.16 (t, J=6.8 Hz, 3H). LCMS calc. for C₂₅H₂₉BrClF₃N₇O₄ [M+H]⁺: m/z=662.1; Found: [M+H−100]⁺: 562.4.

### Step 2: tert-butyl 4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazin e-1-carboxylate

A mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5a]pyrimidin-6yl)piperazine-1-carboxylate (290 mg, 0.437 mmol), morpholine (114.34 mg, 1.31 mmol) and KOAc (257.61 mg, 2.62 mmol) in DMSO (2.5 mL) and DMF (2.5 mL) was stirred at 120° C. for 3 h. After it was cooled to r.t., the mixture was diluted with EtOAc (300 mL), washed with sat. NH₄Cl aq. (100 mL×3). The organic layer was dried over Na₂SO₄, filtered and concentrated. The residue was purified by flash chromatography on a silica gel column eluting with EtOAc/PE (70%) to afford the title compound (180 mg, 61% yield) as a white solid. ¹H NMR (400 MHZ, DMSO-d₆) δ ppm 10.33 (s, 1H), 8.04 (d, J=8.5 Hz, 1H), 7.96 (d, J=1.7 Hz, 1H), 7.72 (dd, J=8.7, 1.7 Hz, 1H), 5.21 (s, 2H), 3.92 (d, J=11.2 Hz, 2H), 3.69-3.63 (m, 4H), 3.42-3.34 (m, 6H), 2.97-2.85 (m, 4H), 2.61 (d, J=10.9 Hz, 2H), 1.43 (s, 9H), 1.16-1.12 (m, 3H). LCMS calc. for C₂₉H₃₇ClF₃N₈O₅ [M+H]⁺: m/z=669.2; Found: [M+H−56]⁺: 614.6.

### Step 3: N-(2-chloro-4-(trifluoromethyl)phenyl)-2-(5-ethyl-2-morpholino-7-oxo-6-(piperazin-1-yl)-[1,2,4]triazolo[1,5-a]pyrimidin-4(7H)-yl)acetamide

A mixture of tert-butyl 4-(4-(2-((2-chloro-4-(trifluoromethyl)phenyl)amino)-2-oxoethyl)-5-ethyl-2-morpholino-7-oxo-4,7-dihydro-[1,2,4]triazolo[1,5-a]pyrimidin-6-yl)piperazine-1-carboxylate (100 mg, 0.149 mmol) in 4 M HCl/dioxane (3 mL) was stirred at r.t. for 2 h., and then concentrated to afford the title compound (95 mg) as a white solid. LCMS calc. for C₂₄H₂₉ClF₃N₈O₃ [M+H]⁺: m/z=569.2; Found: 569.0.

### INT B5: N-(2-Chloro-4-(trifluoromethyl)phenyl)-2-(2-(1,3-dihydro-2H-pyrrolo[3,4-c]pyridin-2-yl)-5-ethyl-7-oxo-6-(piperazin-1-yl)-[1,2,4]triazolo[1,5-a]pyrimidin-4(7H)-yl)acetamide

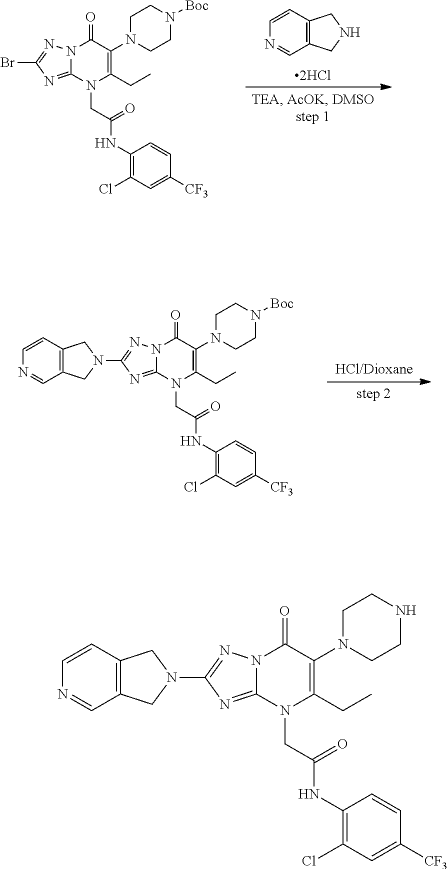 [SMILES: CCc1c(N2CCN(C(=O)OC(C)(C)C)CC2)c(=O)n2nc(Br)nc2n1CC(=O)Nc1ccc(C(F)(F)F)cc1Cl ; c1cc2c(cn1)CNC2 ; CCc1c(N2CCN(C(=O)OC(C)(C)C)CC2)c(=O)n2nc(N3Cc4ccncc4C3)nc2n1CC(=O)Nc1ccc(C(F)(F)F)cc1Cl ; CCc1c(N2CCNCC2)c(=O)n2nc(N3Cc4ccncc4C3)nc2n1CC(=O)Nc1ccc(C(F)(F)F)cc1Cl]

### Step 1: tert-butyl 4-(4-(2-((2-chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazine-1-carboxylate

A solution of 2,3-dihydro-1H-pyrrolo[3,4-c]pyridine dihydrochloride (175 mg, 0.90 mmol) in DMSO (2 mL) was added TEA (0.24 mL, 1.80 mmol) and stirred at r.t. for 5 min. tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (200 mg, 0.30 mmol, INT B4 Step 1) and KOAc (176 mg, 1.80 mmol) were added. The resulting mixture was heated at 120° C. for 16 h., cooled to r.t., quenched with water (0.5 mL) and concentrated under reduced pressure. The residue was purified by Prep-HPLC on a C18 column eluting with MeCN/H₂O (30-50%, with 0.1% FA) to afford the title compound (55 mg, 26% yield) as a red solid. LCMS calc. for C₃₂H₃₆ClF₃N₉O₄ [M+H]⁺: m/z=702.2; Found: 702.7.

### Step 2: N-(2-chloro-4-(trifluoromethyl)phenyl)-2-(2-(1,3-dihydro-2H-pyrrolo[3,4-c]pyridin-2-yl)-5-ethyl-7-oxo-6-(piperazin-1-yl)-[1,2,4]triazolo[1,5-a]pyrimidin-4(7H)-yl)acetamide

A solution of tert-butyl 4-(4-(2-((2-chloro-4-(trifluoromethyl)phenyl)amino)-2-oxoethyl)-2-(1,3-dihydro-2H-pyrrolo[3,4-c]pyridin-2-yl)-5-ethyl-7-oxo-4,7-dihydro-[1,2,4]triazolo[1,5-a]pyrimidin-6-yl)piperazine-1-carboxylate (55 mg, 0.08 mmol) in 4 M HCl/dioxane (0.2 mL) was stirred at r.t. for 1 h. and then concentrated under reduced pressure to afford the title compound (55 mg as a HCl salt) as a yellow solid. LCMS calc. for C₂₇H₂₈ClF₃N₉O₂ [M+H]⁺: m/z=602.2; Found: 602.2.

### INT B6: N-(3-Bromo-5-methylthiophen-2-yl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

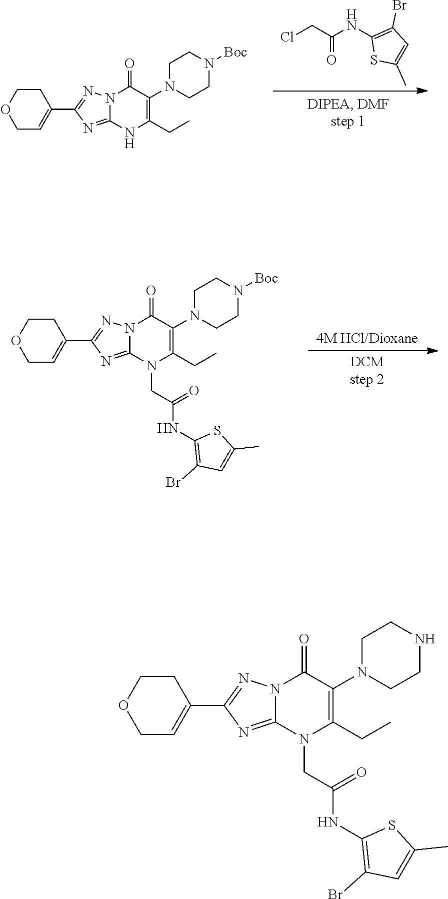 [SMILES: CCc1[nH]c2nc(C3=CCOCC3)nn2c(=O)c1N1CCN(C(=O)OC(C)(C)C)CC1 ; Cc1cc(Br)c(NC(=O)CCl)s1 ; CCc1c(N2CCN(C(=O)OC(C)(C)C)CC2)c(=O)n2nc(C3=CCOCC3)nc2n1CC(=O)Nc1sc(C)cc1Br ; CCc1c(N2CCNCC2)c(=O)n2nc(C3=CCOCC3)nc2n1CC(=O)Nc1sc(C)cc1Br]

### Step 1: tert-butyl 4-(4-(2-((3-bromo-5-methylthiophen-2-yl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

This compound was prepared by procedures analogous to those described for INT B1 Step 6 using INT A3 to replace INT A1 to afford the title product as a white solid. LCMS calc. for C₂₈H₃₇BrN₇O₅S [M+H]⁺: m/z=662.2; Found: 662.0, 664.0.

### Step 2: N-(3-bromo-5-methylthiophen-2-yl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

To a solution of tert-butyl 4-(4-(2-((3-bromo-5-methylthiophen-2-yl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (100 mg, 0.15 mmol) in DCM (1 mL) was added 4 M HCl/dioxane (2 mL). The mixture was stirred at r.t. for 2 h. and concentrated. The residue was washed with EtOAc (5 mL) to obtain the crude product (100 mg) as a HCl salt, which was used in the next step without further purification. LCMS calc. for C₂₃H₂₉BrN₇O₃S [M+H]⁺: m/z=562.1; Found: 562.0.

### INT B7: N-(2-Chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)-N-methylacetamide

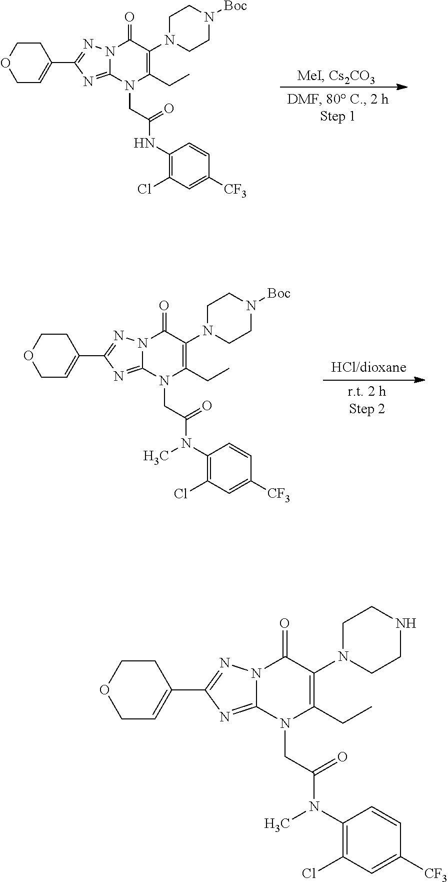 [SMILES: CCc1c(N2CCN(C(=O)OC(C)(C)C)CC2)c(=O)n2nc(C3=CCOCC3)nc2n1CC(=O)Nc1ccc(C(F)(F)F)cc1Cl ; CCc1c(N2CCN(C(=O)OC(C)(C)C)CC2)c(=O)n2nc(C3=CCOCC3)nc2n1CC(=O)N(C)c1ccc(C(F)(F)F)cc1Cl ; CCc1c(N2CCNCC2)c(=O)n2nc(C3=CCOCC3)nc2n1CC(=O)N(C)c1ccc(C(F)(F)F)cc1Cl]

### Step 1: tert-butyl 4-(4-(2-((2-chloro-4-(trifluoromethyl)phenyl)(methyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

A mixture of tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (50 mg, 0.08 mmol, INT B1 Step 6) was added iodomethane (16 mg, 0.11 mmol) and Cs₂CO₃ (73 mg, 0.23 mmol) in DMF (1 mL) was stirred at 80° C. for 2 h. Then the reaction was diluted with water (30 mL), and extracted with EtOAc (10 mL×3). The combined organic layers were dried over anhydrous Na₂SO₄, filtered and concentrated under reduced pressure. The residue was purified by flash chromatography on a silica gel column eluting with MeOH/DCM (30%) to give the compound (50 mg, 98% yield) as a yellow solid. LCMS calc. for C₃₁H₃₈ClF₃N₇O₅ [M+H]⁺: m/z=680.2; Found: [M+H−56]⁺: 624.2.

### Step 2: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)-N-methylacetamide

A mixture of tert-butyl 4-(4-(2-((2-chloro-4-(trifluoromethyl)phenyl)(methyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (50 mg, 0.07 mmol) in 4 M HCl/dioxane (2 mL) was stirred at r.t. for 2 h. The mixture was concentrated and dried to afford the title compound (40 mg, 94% yield) as a yellow solid. LCMS calc. for C₂₆H₃₀ClF₃N₇O₃ [M+H]⁺: m/z=580.2; Found: 580.2.

### INT B8: N-(2-Chloro-4-(trifluoromethyl)phenyl)-2-(2-(3,6-dihydro-2H-pyran-4-yl)-5-ethyl-7-oxo-6-(2,7-diazaspiro[4.4]nonan-2-yl)-[1,2,4]triazolo[1,5-a]pyrimidin-4(7H)-yl)acetamide

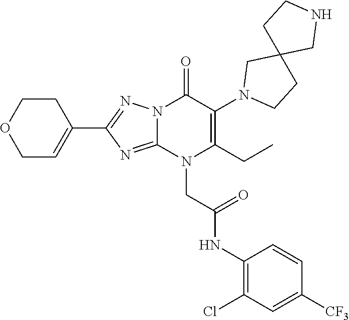 [SMILES: CCc1c(N2CCC3(CCNC3)C2)c(=O)n2nc(C3=CCOCC3)nc2n1CC(=O)Nc1ccc(C(F)(F)F)cc1Cl]

This compound was prepared by procedures analogous to those described for INT B1 Step 3-7 using tert-butyl 2,7-diazaspiro[4.4]nonane-2-carboxylate to replace tert-butyl piperazine-1-carboxylate in Step 3 to afford the title product as a yellow solid. LCMS calc. for C₂₈H₃₂ClF₃N₇O₃ [M+H]⁺: m/z=606.2; Found: 606.3.

### INT B9: N-(2-Chloro-4-(trifluoromethyl)phenyl)-2-(2-(3,6-dihydro-2H-pyran-4-yl)-5-ethyl-6-(hexahydropyrrolo[3,4-c]pyrrol-2 (1H)-yl)-7-oxo-[1,2,4]triazolo[1,5-a]pyrimidin-4(7H)-yl)acetamide

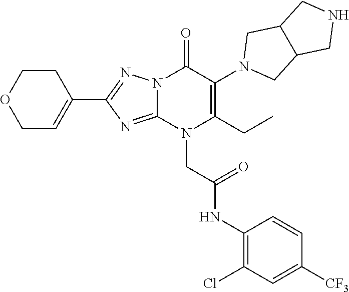 [SMILES: CCc1c(N2CC3CNCC3C2)c(=O)n2nc(C3=CCOCC3)nc2n1CC(=O)Nc1ccc(C(F)(F)F)cc1Cl]

This compound was prepared by procedures analogous to those described for INT B1 Step 3-7 using tert-butyl hexahydropyrrolo[3,4-c]pyrrole-2 (1H)-carboxylate to replace tert-butyl piperazine-1-carboxylate in Step 3 to afford the title product as a light yellow solid. LCMS calc. for C₂₇H₃₀ClF₃N₇O₃ [M+H]⁺: m/z=592.2; Found: 592.4.

### INT B10:2-(6-(4-Aminopiperidin-1-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

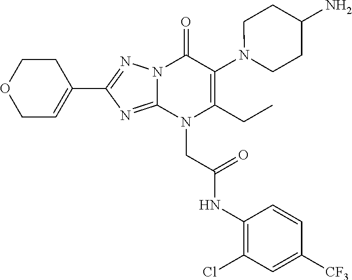 [SMILES: CCc1c(N2CCC(N)CC2)c(=O)n2nc(C3=CCOCC3)nc2n1CC(=O)Nc1ccc(C(F)(F)F)cc1Cl]

This compound was prepared by procedures analogous to those described for INT B1 Step 3-7 using tert-butyl piperidin-4-ylcarbamate to replace tert-butyl piperazine-1-carboxylate in Step 3 to afford the title product as a brown solid. LCMS calc. for C₂₆H₃₀ClF₃N₇O₃ [M+H]⁺: m/z=580.2; Found: 580.2.

### INT B11: N-(2-Chloro-4-(trifluoromethyl)phenyl)-2-(2-(3,6-dihydro-2H-pyran-4-yl)-5-hydroxy-7-oxo-6-(piperazin-1-yl)-[1,2,4]triazolo[1,5-a]pyrimidin-4(7H)-yl)acetamide

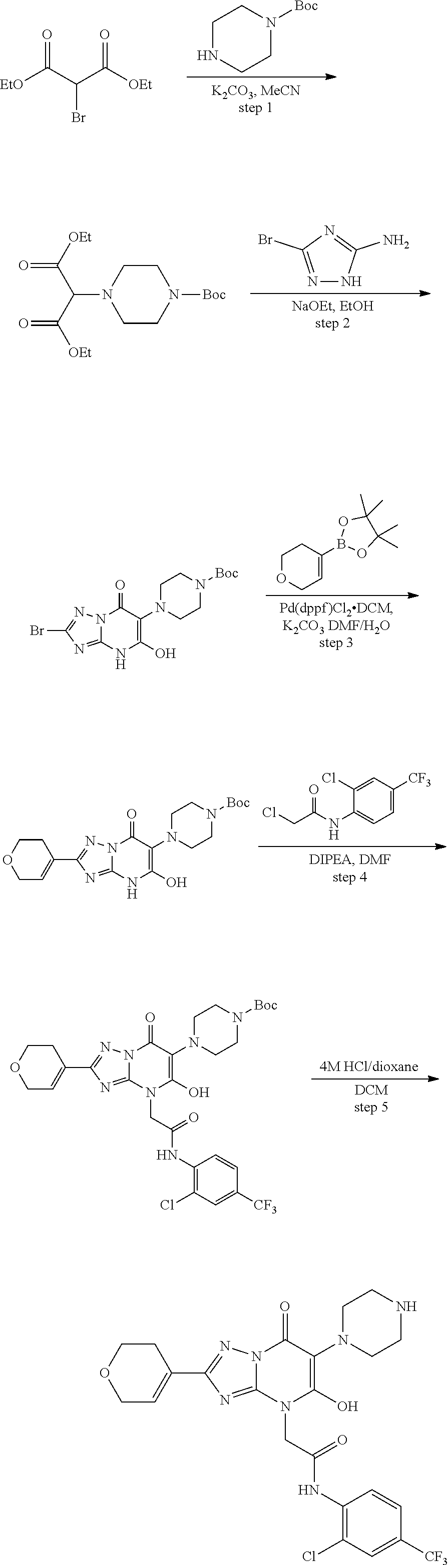

### Step 1: diethyl 2-(4-(tert-butoxycarbonyl)piperazin-1-yl)malonate

A mixture of diethyl 2-bromomalonate (10 g, 41.8 mmol), tert-butyl piperazine-1-carboxylate (15.5 g, 62.8 mmol) and K₂CO₃ (8.67 g, 62.8 mmol) in ACN (100 mL) was stirred for 2 days at 40° C. under N₂ atmosphere. The reaction mixture was diluted with water, and extracted with DCM (10 mL×3). The combined organic layers were concentrated. The residue was purified by flash chromatography on a silica gel column eluting with AcOEt/PE (0˜35%) to afford the title compound (13.5 g, 93% yield) as a white solid. LCMS calc. for C₁₆H₂₉N₂O₆ [M+H]⁺: m/z=345.2; Found: 345.0.

### Step 2: tert-butyl 4-(2-bromo-7-hydroxy-5-oxo-4,5-dihydro-[1,2,4]triazolo[1,5-a]pyrimidin-6-yl)piperazine-1-carboxylate

To a mixture of tert-butyl diethyl 2-(4-(tert-butoxycarbonyl)piperazin-1-yl)malonate (25.4 g, 73.6 mmol) and sodium ethoxide (8.4 g, 122.7 mmol) in EtOH (100 mL) was added 3-bromo-1H-1,2,4-triazol-5-amine (10 g, 61.35 mmol). The mixture was stirred at 100° C. for 2 h. under N₂ atmosphere. The mixture was filtered. The filtrate was diluted with H₂O, adjusted to PH˜4 with HCl aq. (2 N), and extracted with DCM (100 mL×3). The combined organic layers were washed with brine, dried over Na₂SO₄, filtered and concentrated under reduced pressure. The residue was purified by flash chromatography on a silica gel column eluting with MeOH/DCM (0-10%) to afford the title compound (16 g, 64% yield) as a yellow solid. LCMS calc. for C₁₄H₂₀BrN₆O₄ [M+H]⁺: m/z=415.1; Found: 415.2.

### Step 3: tert-butyl 4-(2-(3,6-dihydro-2H-pyran-4-yl)-5-hydroxy-7-oxo-4,7-dihydro-[1,2,4]triazolo[1,5-a]pyrimidin-6-yl)piperazine-1-carboxylate

A mixture of tert-butyl 4-(2-bromo-7-hydroxy-5-oxo-4,5-dihydro-[1,2,4]triazolo[1,5-a]pyrimidin-6-yl)piperazine-1-carboxylate (6 g, 14.5 mmol), (3,6-dihydro-2H-pyran-4-yl)-4,4,5,5-tetramethyl-1,3,2-dioxaborolane (6.2 g, 29 mmol), Pd(dppf)Cl₂ DCM (600 mg, 0.72 mmol) and K₂CO₃ (6 g, 43.5 mmol) in DMF/H₂O (7:3) (60 mL) was degassed and recharged with argon for three cycles, and stirred at 100° C. for 12 h. The reaction mixture was extracted with DCM (100 mL×3). The combined organic layers were washed with brine, dried over Na₂SO₄, filtered and concentrated under reduced pressure. The residue was purified by flash chromatography on a silica gel column eluting with MeOH/DCM (0-20%) to afford the title compound (4.2 g, 69% yield) as a yellow solid. LCMS calc. for C₁₉H₂₇N₆O₅ [M+H]⁺: m/z=419.2; Found: 419.0.

### Step 4: tert-butyl 4-(4-(2-((2-chloro-4-(trifluoromethyl)phenyl)amino)-2-oxoethyl)-2-(3,6-dihydro-2H-pyran-4-yl)-5-hydroxy-7-oxo-4,7-dihydro-[1,2,4]triazolo[1,5-a]pyrimidin-6-yl)piperazine-1-carboxylate

This compound was prepared by procedures analogous to those described for INT B1 Step 6 using tert-butyl 4-(2-(3,6-dihydro-2H-pyran-4-yl)-5,7-dihydroxy-[1,2,4]triazolo[1,5-a]pyrimidin-6-yl)piperazine-1-carboxylate and INT A1 to afford the title product as a white solid. ¹H NMR (400 MHz, DMSO-d₆) δ ppm 10.17 (s, 1H), 8.07 (d, J=8.6 Hz, 1H), 7.94 (d, J=1.6 Hz, 1H), 7.74-7.68 (m, 1H), 6.70 (s, 1H), 4.93 (s, 2H), 4.23 (d, J=2.4 Hz, 2H), 4.02 (s, 3H), 3.79 (t, J=5.3 Hz, 2H), 3.35 (s, 4H), 3.24 (d, J=10.5 Hz, 4H), 1.44 (s, 9H). LCMS calc. for C₂₈H₃₂ClF₃N₇O₆ [M+H]⁺: m/z=654.2; Found: 654.5.

### Step 5: N-(2-chloro-4-(trifluoromethyl)phenyl)-2-(2-(3,6-dihydro-2H-pyran-4-yl)-5-hydroxy-7-oxo-6-(piperazin-1-yl)-[1,2,4]triazolo[1,5-a]pyrimidin-4(7H)-yl)acetamide

A solution of tert-butyl 4-(4-(2-((2-chloro-4-(trifluoromethyl)phenyl)amino)-2-oxoethyl)-2-(3,6-dihydro-2H-pyran-4-yl)-5-hydroxy-7-oxo-4,7-dihydro-[1,2,4]triazolo[1,5-a]pyrimidin-6-yl)piperazine-1-carboxylate (260 mg, 0.04 mmol) in DCM (2 mL) and 4 M HCl/dioxane (2 mL) was stirred at r.t. for 2 h. under N₂ atmosphere. The reaction mixture was concentrated under reduced pressure to afford the title compound (200 mg, crude) as a white solid. LCMS calc. for C₂₃H₂₄ClF₃N₇O₄ [M+H]⁺: m/z=554.1; Found: 554.2.

### INT B12:2-(2-Bromo-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide

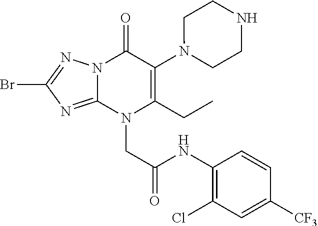 [SMILES: CCc1c(N2CCNCC2)c(=O)n2nc(Br)nc2n1CC(=O)Nc1ccc(C(F)(F)F)cc1Cl]

A mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6yl)piperazine-1-carboxylate (1.66 g, 2.5 mmol, INT B4 Step 1) in 4 M HCl/dioxane (3 mL) was stirred at r.t. for 2 h. The reaction mixture was concentrated to afford the title compound (1.43 g) as a yellow solid. LCMS calc. for C₂₀H₂₁BrClF₃N₇O₂ [M+H]⁺: m/z=562.1; Found: 562.0.

### INT B13:2-(2-(3,6-Dihydro-2H-pyran-4-yl)-5-ethyl-7-oxo-6-(piperazin-1-yl)-[1,2,4]triazolo[1,5-a]pyrimidin-4(7H)-yl)-N-(2-fluoro-4-(trifluoromethyl)phenyl)acetamide

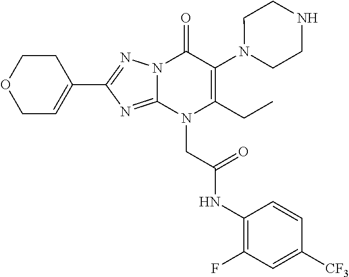 [SMILES: CCc1c(N2CCNCC2)c(=O)n2nc(C3=CCOCC3)nc2n1CC(=O)Nc1ccc(C(F)(F)F)cc1F]

This compound was prepared by procedures analogous to those described for INT B1 Step 6-7 using tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate and 2-chloro-N-(2-fluoro-4-(trifluoromethyl)phenyl)acetamide in Step 6 to afford the title compound (130 mg, crude) as a brown solid. LCMS calc. for C₂₅H₂₈F₄N₇O₃ [M+H]⁺: m/z=550.2; Found: 550.2.

### INT B14: N-(2-Chloro-4-(trifluoromethyl)phenyl)-2-(2-(3,4-dihydro-2H-pyrano[2,3-b]pyridin-6-yl)-5-ethyl-7-oxo-6-(piperazin-1-yl)-[1,2,4]triazolo[1,5-a]pyrimidin-4(7H)-yl)acetamide

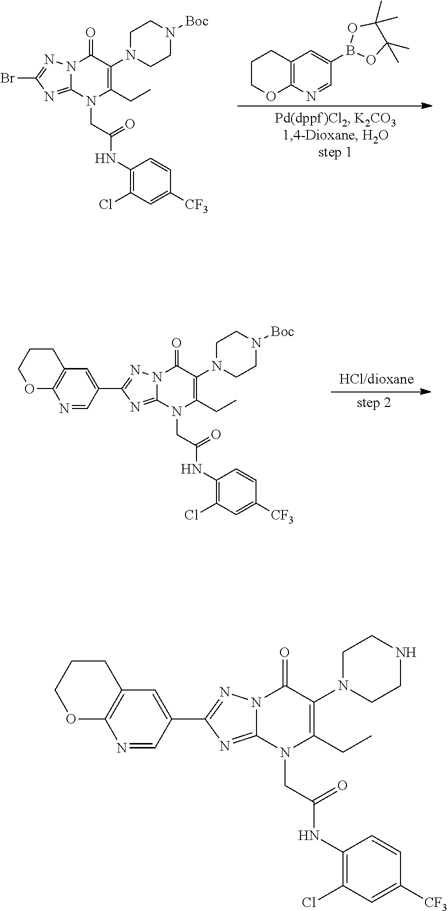 [SMILES: CCc1c(N2CCN(C(=O)OC(C)(C)C)CC2)c(=O)n2nc(Br)nc2n1CC(=O)Nc1ccc(C(F)(F)F)cc1Cl ; CC1(C)OB(c2cnc3c(c2)CCCO3)OC1(C)C ; CCc1c(N2CCN(C(=O)OC(C)(C)C)CC2)c(=O)n2nc(-c3cnc4c(c3)CCCO4)nc2n1CC(=O)Nc1ccc(C(F)(F)F)cc1Cl ; CCc1c(N2CCNCC2)c(=O)n2nc(-c3cnc4c(c3)CCCO4)nc2n1CC(=O)Nc1ccc(C(F)(F)F)cc1Cl]

### Step 1: tert-butyl 4-(4-(2-((2-chloro-4-(trifluoromethyl)phenyl)amino)-2-oxoethyl)-2-(3,4-dihydro-2H-pyrano[2,3-b]pyridin-6-yl)-5-ethyl-7-oxo-4,7-dihydro-[1,2,4]triazolo[1,5-a]pyrimidin-6-yl)piperazine-1-carboxylate

A mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (100 mg, 0.15 mmol, INT B4 step 1), Pd(dppf)Cl₂ (11.1 mg, 0.02 mmol), K₂CO₃ (41.4 mg, 0.30 mmol) and 6-(4,4,5,5-tetramethyl-1,3,2-dioxaborolan-2-yl)-3,4-dihydro-2H-pyrano[2,3-b]pyridine (78.3 mg, 0.30 mmol) in 1,4-dioxane (4 mL) and H₂O (0.5 mL) was stirred at 100° C. under N₂ for 1 h. The reaction mixture was quenched with H₂O (5 mL) and extracted with DCM (5 mL×3). The organic layers were washed with NH₄Cl (aq.) and brine, dried over Na₂SO₄, filtered and concentrated under reduced pressure. The residue was purified by flash chromatography on a silica gel column eluting with MeOH/DCM (10%) to afford the title compound (60 mg, 56.0% yield) as a yellow solid. LCMS calc. for C₃₃H₃₇ClF₃N₈O₅ [M+H]⁺: m/z=717.2; Found: [M+H−56]⁺: 662.2.

### Step 2. N-(2-chloro-4-(trifluoromethyl)phenyl)-2-(2-(3,4-dihydro-2H-pyrano[2,3-b]pyridin-6-yl)-5-ethyl-7-oxo-6-(piperazin-1-yl)-[1,2,4]triazolo[1,5-a]pyrimidin-4(7H)-yl)acetamide

A solution of tert-butyl 4-(4-(2-((2-chloro-4-(trifluoromethyl)phenyl)amino)-2-oxoethyl)-2-(3,4-dihydro-2H-pyrano[2,3-b]pyridin-6-yl)-5-ethyl-7-oxo-4,7-dihydro-[1,2,4]triazolo[1,5-a]pyrimidin-6-yl)piperazine-1-carboxylate (20 mg, 27.9 μmol), in DCM (0.5 mL), 4 M HCl/dioxane (1 mL) was stirred at r.t. for 1 h. The reaction mixture was quenched with H₂O (5 mL) and extracted with DCM (5 mL×3). The organic layers were washed with aq. NH₄Cl and brine, dried over Na₂SO₄, filtered and concentrated under reduced pressure. The residue was purified by flash chromatography on a silica gel column eluting with MeOH/DCM (10%) to afford the title compound (12 mg, 69.7% yield) as a yellow solid. LCMS calc. for C₂₈H₂₉ClF₃N₈O₃ [M+H]⁺: m/z=617.2; Found: 617.5.

### INT B15: 2-(2-(Tert-butyl)-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide

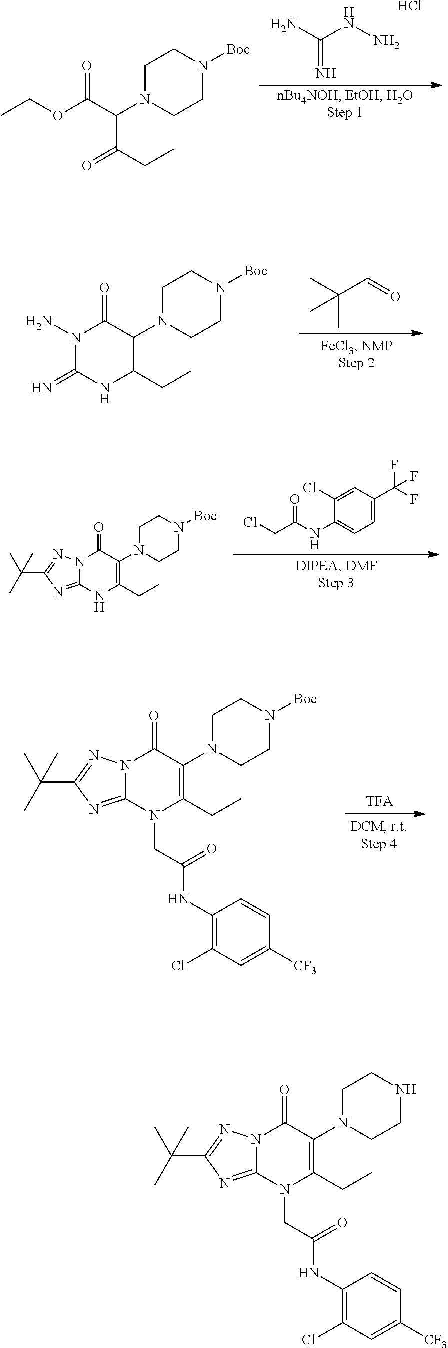

### Step 1: tert-butyl 4-(3-amino-6-ethyl-2-imino-4-oxo-1,2,3,4-tetrahydropyrimidin-5-yl)piperazine-1-carboxylate

To a stirred solution of hydrazinecarboximidamide hydrochloride (1.0 g, 9.15 mmol) in EtOH (20 mL) was added nBu₄NOH (4.7 g, 50% wt in water, 9.15 mmol) at r.t. The mixture was stirred for 1.5 h. at 556° C. Tert-butyl 4-(1-ethoxy-1,3-dioxopentan-2-yl)piperazine-1-carboxylate (1.5 g, 4.57 mmol) was added. The reaction mixture was stirred for 6 h. at 78° C. and concentrated under reduced pressure to the residue was added EtOH (5 mL) and the result suspension was stirred for 1 h. at 0° C. The precipitate was collected by filtration, washed with cold EtOH and dried under reduced pressure to afford the title compound (660 mg, 43% yield) as a white solid. LCMS calc. for C₁₅H₂₇N₆O₃ [M+H]⁺: m/z=339.2; Found: 339.4.

### Step 2: tert-butyl 4-(2-(tert-butyl)-5-ethyl-7-oxo-4,7-dihydro-[1,2,4]triazolo[1,5-a]pyrimidin-6-yl)piperazine-1-carboxylate

To a solution of tert-butyl 4-(3-amino-6-ethyl-2-imino-4-oxo-1,2,3,4-tetrahydropyrimidin-5-yl)piperazine-1-carboxylate (200 mg, 0.59 mmol) and pivalaldehyde (76 mg, 0.89 mmol) in NMP (5 mL) was added FeCl₃ (192 mg, 1.18 mmol) at r.t. The solution was heated to 50° C. and stirred for 16 h. The mixture was diluted with H₂O (20 mL) and extracted with EtOAc (20 mL×3). The combined organic layers were washed with brine, dried over anhydrous Na₂SO₄, filtered, concentrated under reduced pressure, the residue was purified by flash chromatography on a silica gel column eluting with MeOH/DCM (0-5%) to afford the title product (180 mg, 75% yield) as a white solid. LCMS calc. for C₂₀H₃₃N₆O₃ [M+H]⁺: m/z=405.3; Found: 405.5.

### Step 3: tert-butyl 4-(2-(tert-butyl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

This compound was prepared by procedures analogous to those described for INT B1 Step 6 using tert-butyl 4-(2-(tert-butyl)-5-ethyl-7-oxo-4,7-dihydro-[1,2,4]triazolo[1,5-a]pyrimidin-6-yl)piperazine-1-carboxylate and INT A1 to afford the title product as a white solid. LCMS calc. for C₂₉H₃₈ClF₃N₇O₄ [M+H]⁺: m/z=640.3; Found: 640.2.

### Step 4: 2-(2-(tert-butyl)-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide

This compound was prepared by procedures analogous to those described for INT B1 Step 7 using tert-butyl 4-(2-(tert-butyl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate and TFA to afford the title product as a TFA salt, which was used for the next step directly. LCMS calc. for C₂₄H₃₀ClF₃N₇O₂ [M+H]⁺: m/z=540.2; Found: 540.2.

### INT B16: N-(2-Chloro-4-(trifluoromethyl)phenyl)-2-(2-cyclopropyl-5-ethyl-7-oxo-6-(piperazin-1-yl)-[1,2,4]triazolo[1,5-a]pyrimidin-4(7H)-yl)acetamide

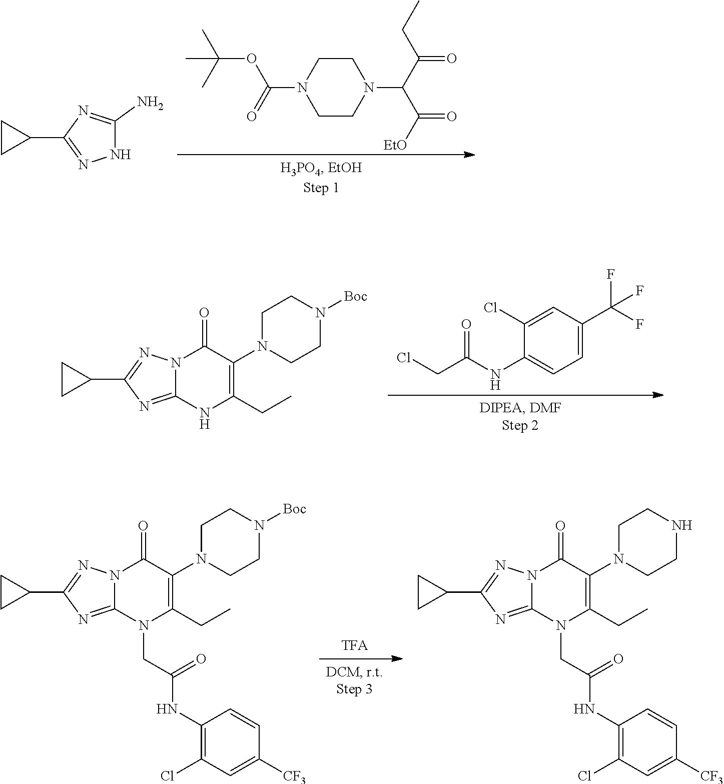 [SMILES: Nc1nc(C2CC2)n[nH]1 ; CCOC(=O)C(C(=O)CC)N1CCN(C(=O)OC(C)(C)C)CC1 ; CCc1[nH]c2nc(C3CC3)nn2c(=O)c1N1CCN(C(=O)OC(C)(C)C)CC1 ; O=C(CCl)Nc1ccc(C(F)(F)F)cc1Cl ; CCc1c(N2CCN(C(=O)OC(C)(C)C)CC2)c(=O)n2nc(C3CC3)nc2n1CC(=O)Nc1ccc(C(F)(F)F)cc1Cl ; CCc1c(N2CCNCC2)c(=O)n2nc(C3CC3)nc2n1CC(=O)Nc1ccc(C(F)(F)F)cc1Cl]

### Step 1: tert-butyl 4-(2-cyclopropyl-5-ethyl-7-oxo-4,7-dihydro-[1,2,4]triazolo[1,5-a]pyrimidin-6-yl)piperazine-1-carboxylate

This compound was prepared by procedures analogous to those described for INT B1 Step 4 using 3-cyclopropyl-1H-1,2,4-triazol-5-amine and tert-butyl 4-(1-ethoxy-1,3-dioxopentan-2-yl)piperazine-1-carboxylate to afford the title product as a white solid. LCMS calc. for C₁₉H₂₉N₆O₃ [M+H]⁺: m/z=389.2; Found: 389.4.

### Step 2: tert-butyl 4-(4-(2-((2-chloro-4-(trifluoromethyl)phenyl)amino)-2-oxoethyl)-2-cyclopropyl-5-ethyl-7-oxo-4,7-dihydro-[1,2,4]triazolo[1,5-a]pyrimidin-6-yl)piperazine-1-carboxylate

This compound was prepared by procedures analogous to those described for INT B1 Step 6 using tert-butyl 4-(2-cyclopropyl-5-ethyl-7-oxo-4,7-dihydro-[1,2,4]triazolo[1,5-a]pyrimidin-6-yl)piperazine-1-carboxylate and INT A1 to afford the title product as a white solid. LCMS calc. for C₂₈H₃₄ClF₃N₇O₄ [M+H]⁺: m/z=624.2; Found: 624.2.

### Step 3: N-(2-chloro-4-(trifluoromethyl)phenyl)-2-(2-cyclopropyl-5-ethyl-7-oxo-6-(piperazin-1-yl)-[1,2,4]triazolo[1,5-a]pyrimidin-4(7H)-yl)acetamide

This compound was prepared by procedures analogous to those described for INT B1 Step 6 using tert-butyl 4-(4-(2-((2-chloro-4-(trifluoromethyl)phenyl)amino)-2-oxoethyl)-2-cyclopropyl-5-ethyl-7-oxo-4,7-dihydro-[1,2,4]triazolo[1,5-a]pyrimidin-6-yl)piperazine-1-carboxylate and TFA to afford the title product as a TFA salt. LCMS calc. for C₂₃H₂₆ClF₃N₇O₂ [M+H]⁺: m/z=524.2; Found: 524.2.

### INT B17: N-(2-chloro-4-(trifluoromethyl)phenyl)-2-(2-ethoxy-5-ethyl-7-oxo-6-(piperazin-1-yl)-[1,2,4]triazolo[1,5-a]pyrimidin-4(7H)-yl)acetamide

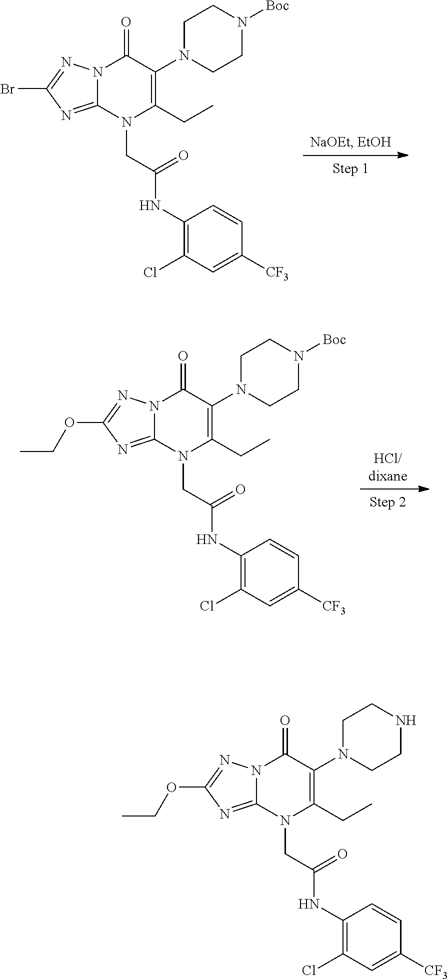 [SMILES: CCc1c(N2CCN(C(=O)OC(C)(C)C)CC2)c(=O)n2nc(Br)nc2n1CC(=O)Nc1ccc(C(F)(F)F)cc1Cl ; CCOc1nc2n(CC(=O)Nc3ccc(C(F)(F)F)cc3Cl)c(CC)c(N3CCN(C(=O)OC(C)(C)C)CC3)c(=O)n2n1 ; CCOc1nc2n(CC(=O)Nc3ccc(C(F)(F)F)cc3Cl)c(CC)c(N3CCNCC3)c(=O)n2n1]

### Step 1: tert-butyl 4-(4-(2-((2-chloro-4-(trifluoromethyl)phenyl)amino)-2-oxoethyl)-2-ethoxy-5-ethyl-7-oxo-4,7-dihydro-[1,2,4]triazolo[1,5-a]pyrimidin-6-yl)piperazine-1-carboxylate

A solution of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (400 mg, 0.60 mmol, INT B4 step 1) in DMSO (10 mL), NaOEt 25% in EtOH (310 mg) was stirred at r.t. for 16 h. The reaction mixture was concentrated and the residue was diluted with water (20 mL) and extracted with EtOAc (30 mL×3), washed with NH₄Cl (30 mL×3), dried over with Na₂SO₄. The organic layers were filtered and concentrated, the residue was purified by flash chromatography on a silica gel column eluting with EtOAc/PE (59%) to get the title compound (109 mg, 28.76% yield) as a white solid. LCMS calc. for C₂₇H₃₄ClF₃N₇O₅ [M+H]⁺: m/z=628.2; Found: 628.4.

### Step 2: N-(2-chloro-4-(trifluoromethyl)phenyl)-2-(2-ethoxy-5-ethyl-7-oxo-6-(piperazin-1-yl)-[1,2,4]triazolo[1,5-a]pyrimidin-4(7H)-yl)acetamide

A solution of tert-butyl 4-(4-(2-((2-chloro-4-(trifluoromethyl)phenyl)amino)-2-oxoethyl)-2-ethoxy-5-ethyl-7-oxo-4,7-dihydro-[1,2,4]triazolo[1,5-a]pyrimidin-6-yl)piperazine-1-carboxylate (109 mg, 0.17 mmol) in 4 M HCl/dioxane (4 mL) and DCM (2 mL) was stirred at r.t. for 1 h. The reaction mixture was concentrated to get the title compound (90 mg, crude HCl salt) as a white solid. LCMS calc. for C₂₂H₂₆ClF₃N₇O₃ [M+H]⁺: m/z=528.2; Found: 528.3.

### INT B18: N-(2-Chloro-4-(trifluoromethyl)phenyl)-2-(5-ethyl-2-(2-methoxyethoxy)-7-oxo-6-(piperazin-1-yl)-[1,2,4]triazolo[1,5-a]pyrimidin-4(7H)-yl)acetamide

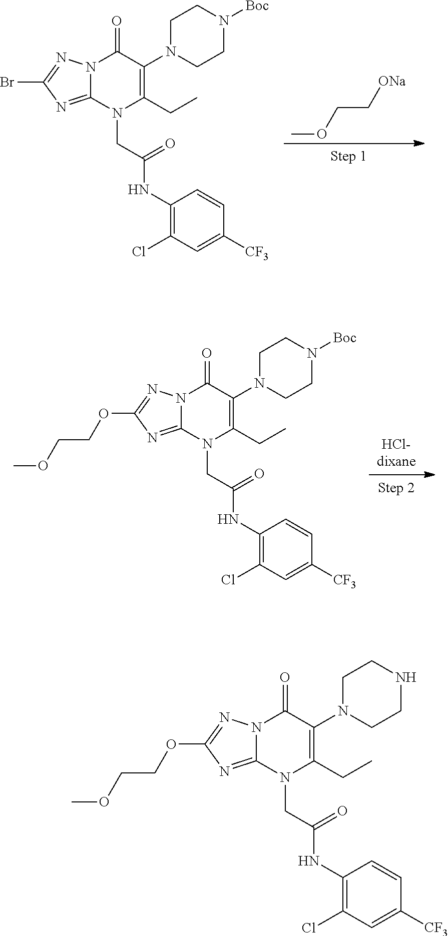

### Step 1: tert-butyl 4-(4-(2-((2-chloro-4-(trifluoromethyl)phenyl)amino)-2-oxoethyl)-5-ethyl-2-(2-methoxyethoxy)-7-oxo-4,7-dihydro-[1,2,4]triazolo[1,5-a]pyrimidin-6-yl)piperazine-1carboxylate

A mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (400 mg, 0.61 mmol) in 2 M sodium 2-methoxyethan-1-olate in 2-methoxyethan-1-ol was stirred at r.t. for 16 h. under Ar. The reaction solution was purified by Prep-HPLC on a C18 column eluting with MeCN/H₂O (70-80%, with 0.05% NH₄HCO₃) to afford the title product (20 mg, 5.03% yield) as a white solid. LCMS calc. for C₂₈H₃₆ClF₃N₇O₆ [M+H]⁺: m/z=658.2; Found: 658.2.

### Step 2: N-(2-chloro-4-(trifluoromethyl)phenyl)-2-(5-ethyl-2-(2-methoxyethoxy)-7-oxo-6-(piperazin-1-yl)-[1,2,4]triazolo[1,5-a]pyrimidin-4(7H)-yl)acetamide

To a solution of tert-butyl 4-(4-(2-((2-chloro-4-(trifluoromethyl)phenyl)amino)-2-oxoethyl)-5-ethyl-2-(2-methoxyethoxy)-7-oxo-4,7-dihydro-[1,2,4]triazolo[1,5-a]pyrimidin-6-yl)piperazine-1-carboxylate (20 mg, 0.03 mmol) in DCM (2 mL) was added 4 M HCl/dioxane (2 mL) was added at 0° C. The mixture was stirred for 3 h at r.t. The reaction mixture was concentrated to get the title product (30 mg, crude) as a yellow solid. LCMS calc. for C₂₃H₂₈ClF₃N₇O₄ [M+H]⁺: m/z=558.2; Found: 558.2.

### INT B19: N-(2-Chloro-4-(trifluoromethyl)phenyl)-2-(5-ethyl-2-(methylthio)-7-oxo-6-(piperazin-1-yl)-[1,2,4]triazolo[1,5-a]pyrimidin-4(7H)-yl)acetamide

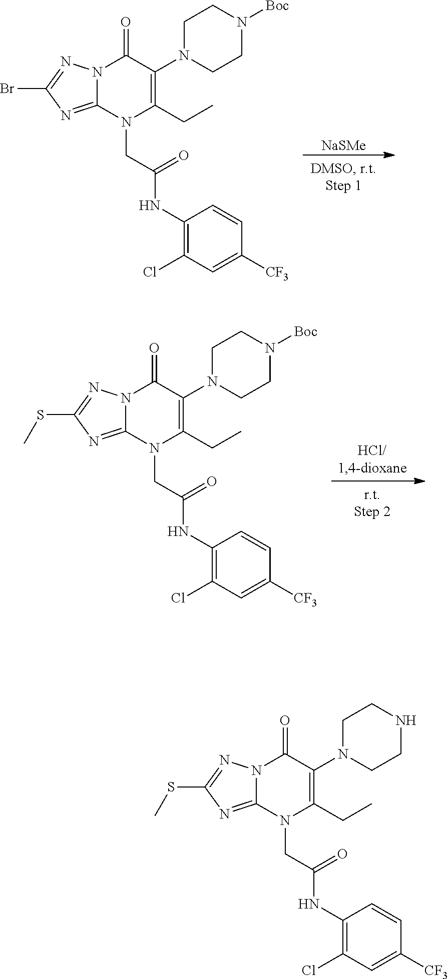 [SMILES: CCc1c(N2CCN(C(=O)OC(C)(C)C)CC2)c(=O)n2nc(Br)nc2n1CC(=O)Nc1ccc(C(F)(F)F)cc1Cl ; CCc1c(N2CCN(C(=O)OC(C)(C)C)CC2)c(=O)n2nc(SC)nc2n1CC(=O)Nc1ccc(C(F)(F)F)cc1Cl ; "NaSMe" ; CCc1c(N2CCNCC2)c(=O)n2nc(SC)nc2n1CC(=O)Nc1ccc(C(F)(F)F)cc1Cl]

This compound was prepared using procedures analogous to those described for INT B17 step 1-2 using tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (INT B4 step 1) and NaSMe in step 1 to afford the title compound as a yellow solid. LCMS calc. for C₂₁H₂₄ClF₃N₇O_{2S} [M+H]⁺: m/z=530.1; Found: 530.4.

### INT B20: N-(2-Chloro-4-(trifluoromethyl)phenyl)-2-(5-ethyl-2-(3-methoxyazetidin-1-yl)-7-oxo-6-(piperazin-1-yl)-[1,2,4]triazolo[1,5-a]pyrimidin-4(7H)-yl)acetamide

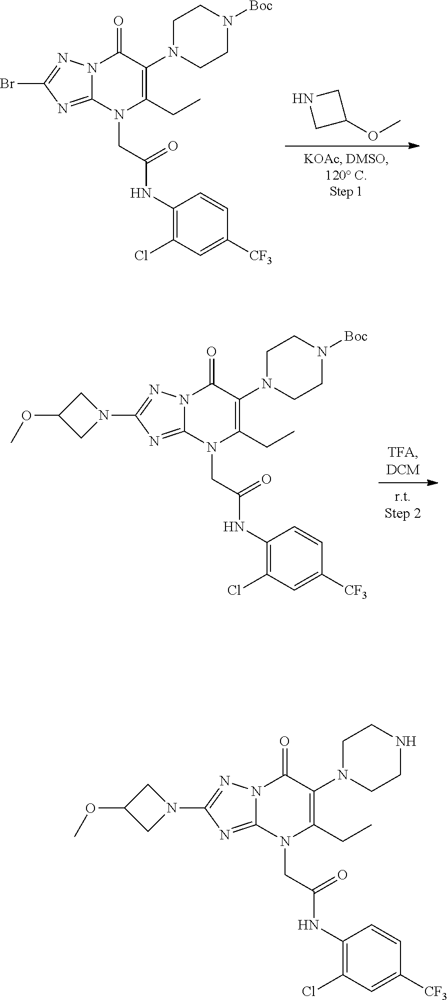

### Step 1: tert-butyl 4-(4-(2-((2-chloro-4-(trifluoromethyl)phenyl)amino)-2-oxoethyl)-5-ethyl-2-(3-methoxyazetidin-1-yl)-7-oxo-4,7-dihydro-[1,2,4]triazolo[1,5-a]pyrimidin-6-yl)piperazine-1-carboxylate

To a mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (200 mg, 0.3 mmol) in DMSO (2 mL) was added 3-methoxyazetidine hydrochloride (74 mg, 0.6 mmol) and potassium acetate (147 mg, 1.5 mmol) at r.t. under nitrogen atmosphere. The resulting mixture was stirred 120° C. for 12 h. The reaction mixture was concentrated under reduced pressure. The residue was purified by flash chromatography on a C18 column eluting with MeCN/H₂O (40-70%) to afford the title compound (180 mg) as a white solid. LCMS calc. for C₂₉H₃₅ClF₃N₈O₅ [M−H]⁻: m/z=667.2; Found: 667.2.

### Step 2: N-(2-chloro-4-(trifluoromethyl)phenyl)-2-(5-ethyl-2-(3-methoxyazetidin-1-yl)-7-oxo-6-(piperazin-1-yl)-[1,2,4]triazolo[1,5-a]pyrimidin-4(7H)-yl)acetamide

To a mixture of tert-butyl 4-(4-(2-((2-chloro-4-(trifluoromethyl)phenyl)amino)-2-oxoethyl)-5-ethyl-2-(3-methoxyazetidin-1-yl)-7-oxo-4,7-dihydro-[1,2,4]triazolo[1,5-a]pyrimidin-6-yl)piperazine-1-carboxylate (180 mg, 0.27 mmol) in DCM (2 mL) was added TFA (0.4 mL, 6.1 mmol) at r.t. The reaction mixture was stirred for 12 h. and concentrated under reduced pressure to afford the title compound (160 mg) as an off-white solid. LCMS calc. for C₂₄H₂₉ClF₃N₈O₃ [M+H]⁺: m/z=569.2; Found: 569.1.

### INT B21: N-(2-Chloro-4-(trifluoromethyl)phenyl)-2-(2-(3-cyanoazetidin-1-yl)-5-ethyl-7-oxo-6-(piperazin-1-yl)-[1,2,4]triazolo[1,5-a]pyrimidin-4(7H)-yl)acetamide

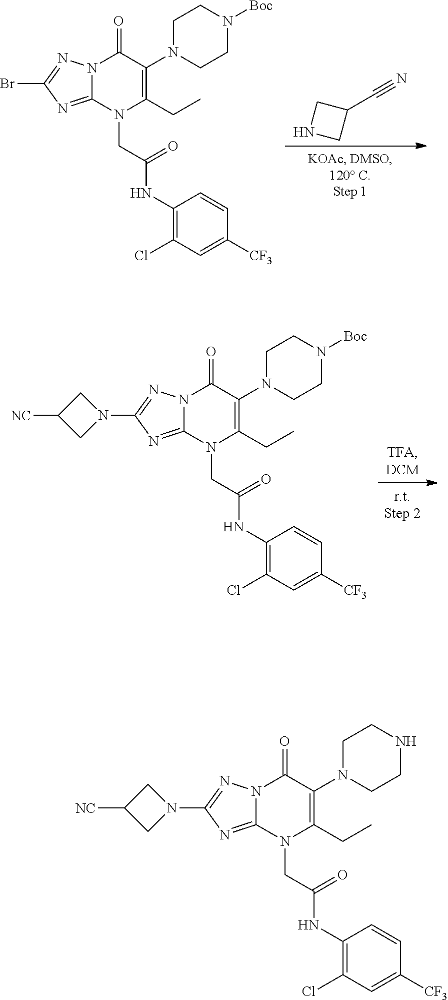 [SMILES: CCc1c(N2CCN(C(=O)OC(C)(C)C)CC2)c(=O)n2nc(Br)nc2n1CC(=O)Nc1ccc(C(F)(F)F)cc1Cl ; N#CC1CNC1 ; CCc1c(N2CCN(C(=O)OC(C)(C)C)CC2)c(=O)n2nc(N3CC(C#N)C3)nc2n1CC(=O)Nc1ccc(C(F)(F)F)cc1Cl ; CCc1c(N2CCNCC2)c(=O)n2nc(N3CC(C#N)C3)nc2n1CC(=O)Nc1ccc(C(F)(F)F)cc1Cl]

This compound was prepared by procedures analogous to those described for INT B20 Step 1-2 using azetidine-3-carbonitrile hydrochloride to replace 3-methoxyazetidine hydrochloride in Step 1 to afford the title compound as an off-white solid. LCMS calc. for C₂₄H₂₆ClF₃N₉O₂ [M+H]⁺: m/z=564.2; Found: 564.1.

### INT B22: N-(2-Chloro-4-(trifluoromethyl)phenyl)-2-(2-(3,3-difluoroazetidin-1-yl)-5-ethyl-7-oxo-6-(piperazin-1-yl)-[1,2,4]triazolo[1,5-a]pyrimidin-4(7H)-yl)acetamide

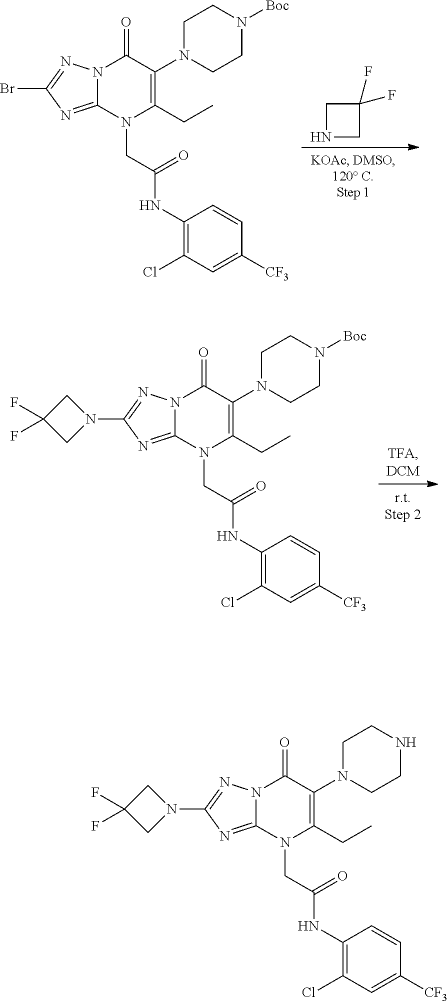 [SMILES: CCc1c(N2CCN(C(=O)OC(C)(C)C)CC2)c(=O)n2nc(Br)nc2n1CC(=O)Nc1ccc(C(F)(F)F)cc1Cl ; FC1(F)CNC1 ; CCc1c(N2CCN(C(=O)OC(C)(C)C)CC2)c(=O)n2nc(N3CC(F)(F)C3)nc2n1CC(=O)Nc1ccc(C(F)(F)F)cc1Cl ; CCc1c(N2CCNCC2)c(=O)n2nc(N3CC(F)(F)C3)nc2n1CC(=O)Nc1ccc(C(F)(F)F)cc1Cl]

This compound was prepared by procedures analogous to those described for INT B20 Step 1-2 using 3,3-difluoroazetidine hydrochloride to replace 3-methoxyazetidine hydrochloride in Step 1 to afford the title compound as an off-white solid. LCMS calc. for C₂₃H₂₃ClF₅N₈O₂ [M−H]⁻: m/z=573.2; Found: 573.1.

### INT B23:2-(5-Ethyl-2-morpholino-7-oxo-6-(piperazin-1-yl)-[1,2,4]triazolo[1,5-a]pyrimidin-4(7H)-yl)-N-(2-fluoro-4-(trifluoromethyl)phenyl)acetamide

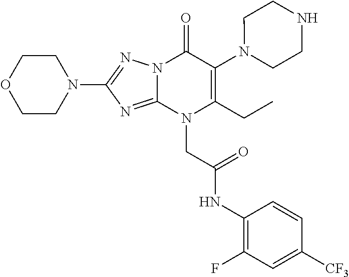 [SMILES: CCc1c(N2CCNCC2)c(=O)n2nc(N3CCOCC3)nc2n1CC(=O)Nc1ccc(C(F)(F)F)cc1F]

This compound was prepared by procedures analogous to those described for INT B4 Step 1-3 using tert-butyl 4-(2-bromo-5-ethyl-7-oxo-4,7-dihydro-[1,2,4]triazolo[1,5-a]pyrimidin-6-yl)piperazine-1-carboxylate (INT B1 step 4) and 2-chloro-N-(2-fluoro-4-(trifluoromethyl)phenyl)acetamide to afford the title compound as a brown solid. LCMS calc. for C₂₄H₂₉F₄N₈O₃ [M+H]⁺: m/z=553.2; Found: 553.2.

### INT B24: (R)-2-(5-Ethyl-7-oxo-2-(3-oxotetrahydro-3H-oxazolo[3,4-a]pyrazin-7 (1H)-yl)-6-(piperazin-1-yl)-[1,2,4]triazolo[1,5-a]pyrimidin-4(7H)-yl)-N-(2-fluoro-4-(trifluoromethyl)phenyl)acetamide

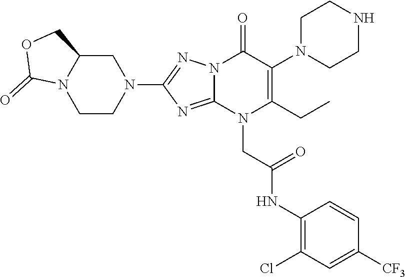 [SMILES: CCc1c(N2CCNCC2)c(=O)n2nc(N3CCN4C(=O)OC[C@H]4C3)nc2n1CC(=O)Nc1ccc(C(F)(F)F)cc1Cl]

### Step 1: tert-butyl 4-(2-bromo-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazine-1-carboxylate

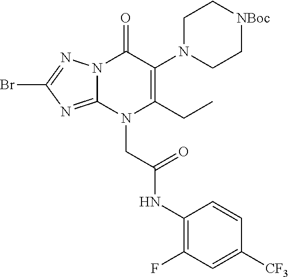 [SMILES: CCc1c(N2CCN(C(=O)OC(C)(C)C)CC2)c(=O)n2nc(Br)nc2n1CC(=O)Nc1ccc(C(F)(F)F)cc1F]

This compound was prepared by procedures analogous to those described for INT B4 Step 1 using tert-butyl 4-(2-bromo-5-ethyl-7-oxo-4,7-dihydro-[1,2,4]triazolo[1,5-a]pyrimidin-6-yl)piperazine-1-carboxylate (INT B1 Step 4) and 2-chloro-N-(2-fluoro-4-(trifluoromethyl)phenyl)acetamide to afford the title compound (110 mg, crude) as a brown solid. LCMS calc. for C₂₅H₂₇BrF₄N₇O₄ [M−H]⁻: m/z=644.1; Found: 644.2.

### Step 2: tert-butyl (R)-4-(5-ethyl-4-(2-((2-fluoro-4-(trifluoromethyl)phenyl)amino)-2-oxoethyl)-7-oxo-2-(3-oxotetrahydro-3H-oxazolo[3,4-a]pyrazin-7 (1H)-yl)-4,7-dihydro-[1,2,4]triazolo[1,5-a]pyrimidin-6-yl)piperazine-1-carboxylate

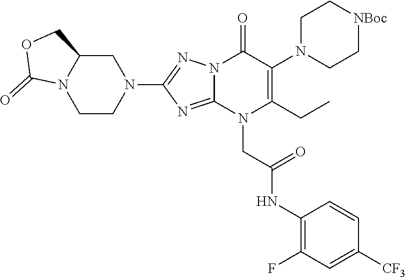 [SMILES: CCc1c(N2CCN(C(=O)OC(C)(C)C)CC2)c(=O)n2nc(N3CCN4C(=O)OC[C@H]4C3)nc2n1CC(=O)Nc1ccc(C(F)(F)F)cc1F]

A mixture of KOAc (100 mg, 1.0 mmol), tert-butyl 4-(2-bromo-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazine-1-carboxylate (32 mg, 0.05 mmol) and (R)-hexahydro-3H-oxazolo[3,4-a]pyrazin-3-one (285 mg, 2.0 mmol) in DMSO (1 mL) was stirred at 110° C. overnight. The mixture was concentrated under reduced pressure. The residue was purified by flash chromatography on a C18 column eluting with eluting with MeCN/H₂O (50-70%) to afford the title compound (24 mg) as a white solid. LCMS calc. for C₃₁H₃₈F₄N₉O₆ [M+H]⁺: m/z=708.3; Found: 708.3.

### Step 3: (R)-2-(5-ethyl-7-oxo-2-(3-oxotetrahydro-3H-oxazolo[3,4-a]pyrazin-7 (1H)-yl)-6-(piperazin-1-yl)-[1,2,4]triazolo[1,5-a]pyrimidin-4(7H)-yl)-N-(2-fluoro-4-(trifluoromethyl)phenyl)acetamide

A mixture of tert-butyl (R)-4-(5-ethyl-4-(2-((2-fluoro-4-(trifluoromethyl)phenyl)amino)-2-oxoethyl)-7-oxo-2-(3-oxotetrahydro-3H-oxazolo[3,4-a]pyrazin-7 (1H)-yl)-4,7-dihydro-[1,2,4]triazolo[1,5-a]pyrimidin-6-yl)piperazine-1-carboxylate (24 mg, 0.04 mmol) in DCM (1 mL) and 4 M HCl/dioxane (0.5 mL) was stirred at r.t. for 2 h. The reaction mixture was concentrated under reduced pressure to afford the title compound (20 mg) as a white solid. LCMS calc. for C₂₆H₃₀F₄N₉O₄ [M+H]⁺: m/z=608.2; Found: 608.2.

### INT B25: N-(2-Chloro-4-(trifluoromethyl)phenyl)-2-(5-ethyl-2-morpholino-7-oxo-6-(piperazin-1-yl)pyrazolo[1,5-a]pyrimidin-4(7H)-yl)acetamide

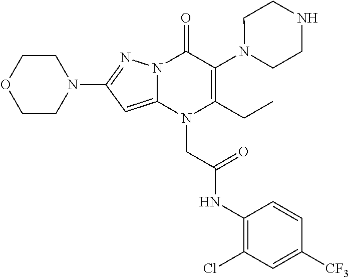 [SMILES: CCc1c(N2CCNCC2)c(=O)n2nc(N3CCOCC3)cc2n1CC(=O)Nc1ccc(C(F)(F)F)cc1Cl]

### Step 1: tert-butyl 4-(5-ethyl-2-morpholino-7-oxo-4,7-dihydropyrazolo[1,5-a]pyrimidin-6-yl)piperazine-1-carboxylate

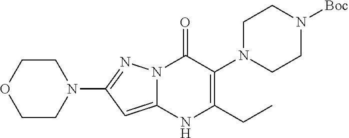 [SMILES: CCc1[nH]c2cc(N3CCOCC3)nn2c(=O)c1N1CCN(C(=O)OC(C)(C)C)CC1]

A mixture of tert-butyl 4-(2-bromo-5-ethyl-7-oxo-4,7-dihydropyrazolo[1,5-a]pyrimidin-6-yl)piperazine-1-carboxylate (600 mg, 1.41 mmol, INT B3 step 1), morpholine (245 mg, 2.81 mmol), t-BuONa (406 mg, 4.22 mmol), Pd₂ (dba) 3 (129 mg, 0.14 mmol), Xphos (201 mg, 0.42 mmol) in dioxane (10 mL) was degassed and recharged with N₂ for 3 cycles, and stirred at 100° C. for 12 h. The mixture was cooled to 25° C., the reaction mixture was diluted with H₂O (10 mL), extracted with EtOAc (10 mL×3). The combined organic layers were washed with brine (5 mL), dried over Na₂SO₄, filtered and concentrated under reduced pressure. The residue was purified by flash chromatography on a silica gel column eluting with MeOH/DCM (5-8%) to afford the title compound (300 mg, 49% yield) as a brown solid. LCMS calc. for C₂₁H₃₂N₆O₄ [M+H]⁺: m/z=433.3; Found: 433.5.

### Step 2: tert-butyl 4-(5-ethyl-4-(2-methoxy-2-oxoethyl)-2-morpholino-7-oxo-4,7-dihydropyrazolo[1,5-a]pyrimidin-6-yl)piperazine-1-carboxylate

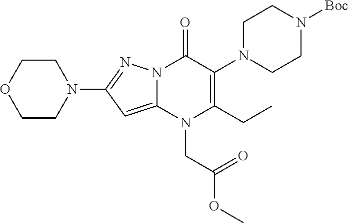 [SMILES: CCc1c(N2CCN(C(=O)OC(C)(C)C)CC2)c(=O)n2nc(N3CCOCC3)cc2n1CC(=O)OC]

This compound was prepared using procedures analogous to those described for INT B3 step 3 using tert-butyl 4-(5-ethyl-2-morpholino-7-oxo-4,7-dihydropyrazolo[1,5-a]pyrimidin-6-yl)piperazine-1-carboxylate and methyl 2-bromoacetate to afford the title compound (300 mg) as a brown solid. LCMS calc. for C₂₄H₃₆N₆O₆ [M+H]⁺: m/z=505.3; Found: 504.6.

### Step 3: 2-(6-(4-(tert-butoxycarbonyl)piperazin-1-yl)-5-ethyl-2-morpholino-7-oxopyrazolo[1,5-a]pyrimidin-4(7H)-yl)acetic acid

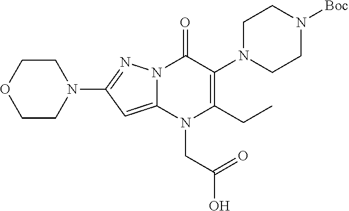 [SMILES: CCc1c(N2CCN(C(=O)OC(C)(C)C)CC2)c(=O)n2nc(N3CCOCC3)cc2n1CC(=O)O]

This compound was prepared using procedures analogous to those described for INT B3 step 4 using tert-butyl 4-(5-ethyl-4-(2-methoxy-2-oxoethyl)-2-morpholino-7-oxo-4,7-dihydropyrazolo[1,5-a]pyrimidin-6-yl)piperazine-1-carboxylate and LiOH·H₂O to afford the title compound (100 mg) as a brown solid. LCMS calc. for C₂₃H₃₄N₆O₆ [M+H]⁺: m/z=491.3; Found: 491.6.

### Step 4: tert-butyl 4-(4-(2-((2-chloro-4-(trifluoromethyl)phenyl)amino)-2-oxoethyl)-5-ethyl-2-morpholino-7-oxo-4,7-dihydropyrazolo[1,5-a]pyrimidin-6-yl)piperazine-1-carboxylate

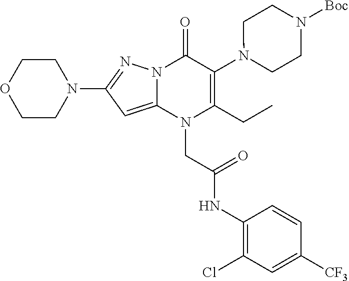 [SMILES: CCc1c(N2CCN(C(=O)OC(C)(C)C)CC2)c(=O)n2nc(N3CCOCC3)cc2n1CC(=O)Nc1ccc(C(F)(F)F)cc1Cl]

To a solution of 2-(6-(4-(tert-butoxycarbonyl)piperazin-1-yl)-5-ethyl-2-morpholino-7-oxopyrazolo[1,5-a]pyrimidin-4(7H)-yl)acetic acid (100 mg, 0.20 mmol) in DCM (2 mL) was added 2-chloro-4-(trifluoromethyl) aniline (80 mg, 0.41 mmol), pyridine (48 mg, 0.61 mmol). The mixture was stirred at 0° C. and added dropwise POCl₃ (47 mg, 0.11 mmol). The mixture was stirred at 0° C. for 1 h. The mixture was diluted with NaHCO₃ (10%, 10 mL), extracted with DCM (10 mL×3). The combined organic layers were washed with brine (5 mL), dried over Na₂SO₄, filtered and concentrated under reduced pressure. The residue was purified by Prep-HPLC eluting with MeCN/H₂O (70%) to afford the title compound (70 mg, 51% yield) as a brown solid. LCMS calc. for C₃₀H₃₇ClF₃N₇O₅ [M+H]⁺: m/z=668.2; Found: 668.1.

### Step 5: N-(2-chloro-4-(trifluoromethyl)phenyl)-2-(5-ethyl-2-morpholino-7-oxo-6-(piperazin-1-yl)pyrazolo[1,5-a]pyrimidin-4(7H)-yl)acetamide

This compound was prepared using procedures analogous to those described for INT B3 step 4 using tert-butyl 4-(4-(2-((2-chloro-4-(trifluoromethyl)phenyl)amino)-2-oxoethyl)-5-ethyl-2-morpholino-7-oxo-4,7-dihydropyrazolo[1,5-a]pyrimidin-6-yl)piperazine-1-carboxylate and TFA to afford the title product as a brown solid. LCMS calc. for C₂₅H₂₉ClF₃N₇O₃ [M+H]⁺: m/z=568.2; Found: 568.0.

### INT B26:2-(2-(5,6-Dihydroimidazo[1,5-a]pyrazin-7 (8H)-yl)-5-ethyl-7-oxo-6-(piperazin-1-yl)-[1,2,4]triazolo[1,5-a]pyrimidin-4(7H)-yl)-N-(2-fluoro-4-(trifluoromethyl)phenyl)acetamide

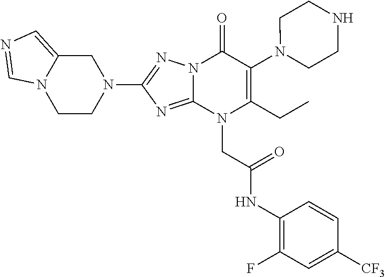 [SMILES: CCc1c(N2CCNCC2)c(=O)n2nc(N3CCn4cncc4C3)nc2n1CC(=O)Nc1ccc(C(F)(F)F)cc1F]

This compound was prepared by procedures analogous to those described for INT B24 Step 2-3 using tert-butyl 4-(2-bromo-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazine-1-carboxylate (INT B24 Step 1) and 5,6,7,8-tetrahydroimidazo[1,5-a]pyrazine in Step 2 to afford the title product as a white solid. LCMS calc. for C₂₆H₂₉F₄N₁₀O₂ [M+H]⁺: m/z=589.2; Found: 589.2.

### INT B27:2-(5-Ethyl-2-(1,4-oxazepan-4-yl)-7-oxo-6-(piperazin-1-yl)-[1,2,4]triazolo[1,5-a]pyrimidin-4(7H)-yl)-N-(2-fluoro-4-(trifluoromethyl)phenyl)acetamide

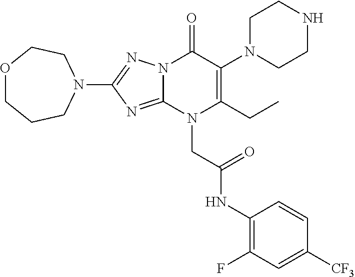 [SMILES: CCc1c(N2CCNCC2)c(=O)n2nc(N3CCCOCC3)nc2n1CC(=O)Nc1ccc(C(F)(F)F)cc1F]

This compound was prepared by procedures analogous to those described for INT B24 Step 2-3 using tert-butyl 4-(2-bromo-5-ethyl-4-(2-((2-fluoro-4-(trifluoromethyl)phenyl)amino)-2-oxoethyl)-7-oxo-4,7-dihydro-[1,2,4]triazolo[1,5-a]pyrimidin-6-yl)piperazine-1-carboxylate (INT B24 Step 1) and 1,4-oxazepane in Step 2 to afford the title product as a white solid. LCMS calc. for C₂₅H₃₁F₄N₈O₃ [M+H]⁺: m/z=567.2; Found: 567.2.

### INT B28:2-(5-Ethyl-2-(1-methyl-4,6-dihydropyrrolo[3,4-c]pyrazol-5 (1H)-yl)-7-oxo-6-(piperazin-1-yl)-[1,2,4]triazolo[1,5-a]pyrimidin-4(7H)-yl)-N-(4-methylthiophen-2-yl)acetamide

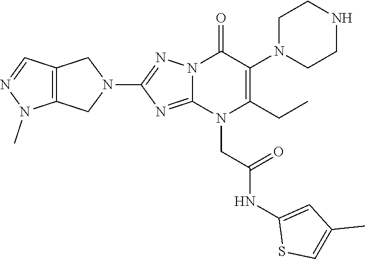 [SMILES: CCc1c(N2CCNCC2)c(=O)n2nc(N3Cc4cnn(C)c4C3)nc2n1CC(=O)Nc1cc(C)cs1]

### Step 1: tert-butyl 4-(2-bromo-5-ethyl-4-(2-((4-methylthiophen-2-yl)amino)-2-oxoethyl)-7-oxo-4,7-dihydro-[1,2,4]triazolo[1,5-a]pyrimidin-6-yl)piperazine-1-carboxylate

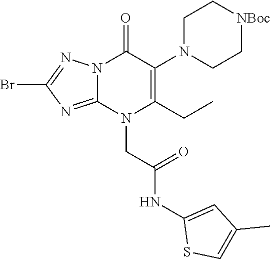 [SMILES: CCc1c(N2CCN(C(=O)OC(C)(C)C)CC2)c(=O)n2nc(Br)nc2n1CC(=O)Nc1cc(C)cs1]

This compound was prepared by procedures analogous to those described for INT B4 Step 1 using tert-butyl 4-(2-bromo-5-ethyl-7-oxo-4,7-dihydro-[1,2,4]triazolo[1,5-a]pyrimidin-6-yl)piperazine-1-carboxylate and 2-chloro-N-(4-methylthiophen-2-yl)acetamide to afford the title compound (120 mg, crude) as a brown solid. LCMS calc. for C₂₃H₃₁BrN₇O₄S [M+H]⁺: m/z=580.1; Found: 580.2.

### Step 2: tert-butyl 4-(5-ethyl-2-(1-methyl-4,6-dihydropyrrolo[3,4-c]pyrazol-5 (1H)-yl)-4-(2-((4-methylthiophen-2-yl)amino)-2-oxoethyl)-7-oxo-4,7-dihydro-[1,2,4]triazolo[1,5-a]pyrimidin-6-yl)piperazine-1-carboxylate

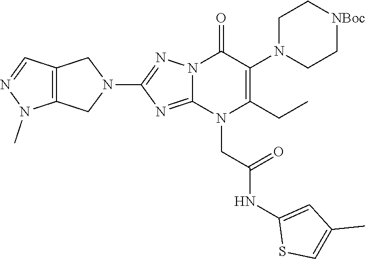 [SMILES: CCc1c(N2CCN(C(=O)OC(C)(C)C)CC2)c(=O)n2nc(N3Cc4cnn(C)c4C3)nc2n1CC(=O)Nc1cc(C)cs1]

A mixture of KOAc (100 mg, 1.0 mmol), tert-butyl 4-(2-bromo-5-ethyl-4-(2-((4-methylthiophen-2-yl)amino)-2-oxoethyl)-7-oxo-4,7-dihydro-[1,2,4]triazolo[1,5-a]pyrimidin-6-yl)piperazine-1-carboxylate (59 mg, 0.1 mmol) and 1-methyl-1,4,5,6-tetrahydropyrrolo[3,4-c]pyrazole (246 mg, 2.0 mmol) in DMSO (1 mL) was stirred at 110° C. overnight. The mixture was concentrated under reduced pressure. The residue was purified by flash chromatography on a C18 column eluting with eluting with MeCN/H₂O (50-70%) to afford the title compound (50 mg) as a white solid. LCMS calc. for C₂₉H₃₇N₁₀O₄S [M−H]⁻: m/z=621.3; Found: 621.2.

### Step 3: 2-(5-ethyl-2-(1-methyl-4,6-dihydropyrrolo[3,4-c]pyrazol-5 (1H)-yl)-7-oxo-6-(piperazin-1-yl)-[1,2,4]triazolo[1,5-a]pyrimidin-4(7H)-yl)-N-(4-methylthiophen-2-yl)acetamide

To a mixture of tert-butyl 4-(5-ethyl-2-(1-methyl-4,6-dihydropyrrolo[3,4-c]pyrazol-5 (1H)-yl)-4-(2-((4-methylthiophen-2-yl)amino)-2-oxoethyl)-7-oxo-4,7-dihydro-[1,2,4]triazolo[1,5-a]pyrimidin-6-yl)piperazine-1-carboxylate (50 mg, 0.08 mmol) in DCM (1 mL) was added TFA (0.5 mL) at r.t. and was stirred for 2 h. The reaction mixture was concentrated under reduced pressure to afford the title compound (30 mg, crude) as a light brown solid. LCMS calc. for C₂₄H₃₁N₁₀O₂S [M+H]⁺: m/z=523.2. Found: 523.2.

### INT B29:2-(2-(3,6-Dihydro-2H-pyran-4-yl)-5-ethyl-7-oxo-6-(piperazin-1-yl)-[1,2,4]triazolo[1,5-a]pyrimidin-4(7H)-yl)-N-(3,5-dimethylthiophen-2-yl)acetamide

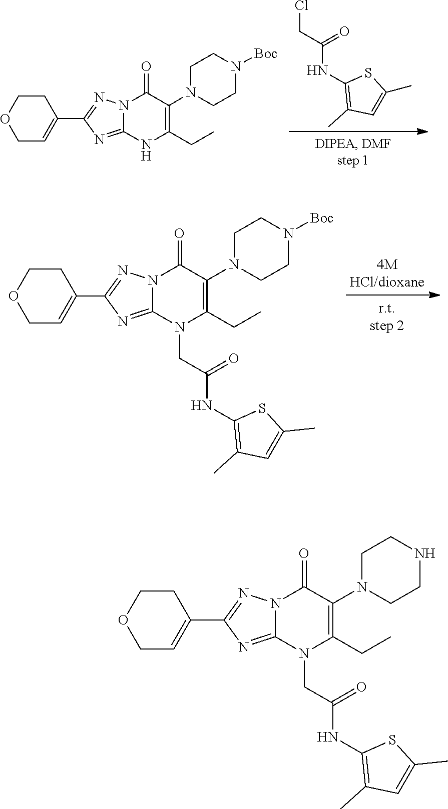 [SMILES: CCc1[nH]c2nc(C3=CCOCC3)nn2c(=O)c1N1CCN(C(=O)OC(C)(C)C)CC1 ; Cc1cc(C)c(NC(=O)CCl)s1 ; CCc1c(N2CCN(C(=O)OC(C)(C)C)CC2)c(=O)n2nc(C3=CCOCC3)nc2n1CC(=O)Nc1sc(C)cc1C ; CCc1c(N2CCNCC2)c(=O)n2nc(C3=CCOCC3)nc2n1CC(=O)Nc1sc(C)cc1C]

### Step 1: tert-butyl 4-(2-(3,6-dihydro-2H-pyran-4-yl)-4-(2-((3,5-dimethylthiophen-2-yl)amino)-2-oxoethyl)-5-ethyl-7-oxo-4,7-dihydro-[1,2,4]triazolo[1,5-a]pyrimidin-6-yl)piperazine-1-carboxylate

This compound was prepared by procedures analogous to those described for INT B1 Step 6 using INT A4 to replace INT A1 to afford the title product as a brown solid. LCMS calc. for C₂₉H₄₀N₇O₅S [M+H]⁺: m/z=598.2; Found: [M+H−56]⁺: 542.2.

### Step 2: 2-(2-(3,6-dihydro-2H-pyran-4-yl)-5-ethyl-7-oxo-6-(piperazin-1-yl)-[1,2,4]triazolo[1,5-a]pyrimidin-4(7H)-yl)-N-(3,5-dimethylthiophen-2-yl)acetamide

The solution of tert-butyl 4-(4-(2-((3-methy-5-methylthiophen-2-yl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (90 mg, 0.15 mmol) in DCM (1 mL) was added 4 M HCl/dioxane (0.4 mL) was stirred at r.t. for 2 h. The reaction mixture was filtered and washed with ethyl acetate to obtain the crude product (100 mg, 0.18 mmol) as HCl salt, which was used in the next step. LCMS calc. for C₂₄H₃₂N₇O₃S [M+H]⁺: m/z=498.2; Found: 498.2.

### INT C1: 4-Hydroxy-1-(4-methoxybenzyl)-1H-pyrazolo[3,4-b]pyridine-5-carboxylic acid

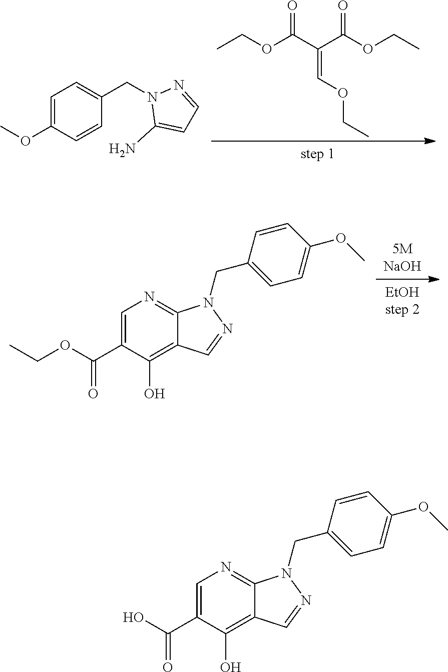 [SMILES: COc1ccc(Cn2nccc2N)cc1 ; CCOC=C(C(=O)OCC)C(=O)OCC ; CCOC(=O)c1cnc2c(cnn2Cc2ccc(OC)cc2)c1O ; COc1ccc(Cn2ncc3c(O)c(C(=O)O)cnc32)cc1]

### Step 1: ethyl 4-hydroxy-1-(4-methoxybenzyl)-1H-pyrazolo[3,4-b]pyridine-5-carboxylate

A mixture of 1-(4-methoxybenzyl)-1H-pyrazol-5-amine (200 mg, 0.985 mmol) and diethyl 2-(ethoxymethylene)malonate (0.5 mL, 1 mmol) was stirred at 130° C. for 45 min. Then diphenyl ether (0.5 mL) was added and heated to 240° C. for 2 h. while ethanol formed in the reaction was removed. The reaction mixture was then cooled to r.t., and diethyl ether (100 mL) was added. The precipitate formed was collected by filtration and dried under vacuum to afford the title compound (130 mg, 40%) as a white solid. LCMS calc. for C₁₇H₁₈N₃O₄ [M+H]⁺: m/z=328.1; Found: 328.2.

### Step 2: 4-hydroxy-1-(4-methoxybenzyl)-1H-pyrazolo[3,4-b]pyridine-5-carboxylic acid

To a solution of ethyl 4-hydroxy-1-(4-methoxybenzyl)-1H-pyrazolo[3,4-b]pyridine-5-carboxylate (50 mg) in ethanol (2 mL) was added aq. sodium hydroxide (5 M, 6 mL). The resulting mixture was stirred at 70° C. for 2 h., and was neutralized with HCl aq. (5 M, 6 mL) at 0° C. The precipitate formed was collected by filtration and washed with water. The solid obtained was suspended in a methanol-toluene and concentrated under reduced pressure to afford the title compound (32 mg, 50%) as colorless powder. LCMS calc. for C₁₅H₁₄N₃O₄ [M+H]⁺: m/z=300.1; Found: 300.1.

### INT C2: 4-Hydroxy-6-methyl-2-oxo-2H-pyran-3-carboxylic acid

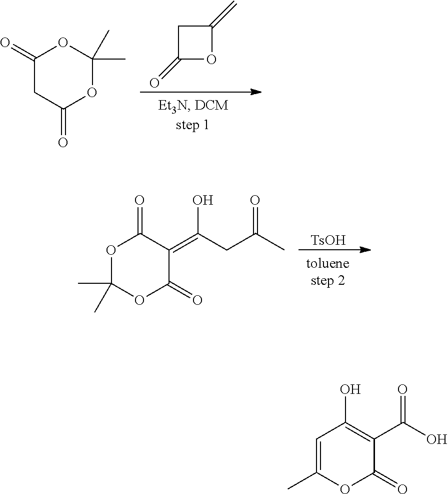

### Step 1: 5-(1-hydroxy-3-oxobutylidene)-2,2-dimethyl-1,3-dioxane-4,6-dione

To a stirring solution of 2,2-dimethyl-1,3-dioxane-4,6-dione (1 g, 6.94 mmol) and Et₃N (0.7 g, 6.94 mmol) in DCM (20 mL) was slowly added 4-methyleneoxetan-2-one (0.7 g, 8.33 mmol) at 0° C. under N₂. The resulting mixture was stirred at r.t. for additional 2 h. The mixture was slowly neutralized with HCl aq. (1 N) at 0° C., and extracted with EtOAc (50 mL×3). The combined organic layers were concentrated under reduced pressure to afford the title compound (1.4 g, 88.5%) as a yellow solid without further purification. LCMS calc. for C₁₀H₁₃O₆ [M+H]⁺: m/z=229.1; Found: 229.2.

### Step 2: 4-hydroxy-6-methyl-2-oxo-2H-pyran-3-carboxylic acid

A mixture of 5-(1-hydroxy-3-oxobutylidene)-2,2-dimethyl-1,3-dioxane-4,6-dione (1 g, 5.65 mmol) and TsOH (56 mg, 0.31 mmol) in toluene (15 mL) was stirred at 110° C. for 2.5 h. under N₂. The mixture was concentrated under reduced pressure. The precipitate was collected to afford the title compound (600 mg, 57.5%) as a yellow solid. LCMS calc. for C₇H₇O₅ [M+H]⁺: m/z=171.0; Found: 171.2.

### INT C3: 3-Aminothieno[2,3-c]pyridine-2-carboxylic acid

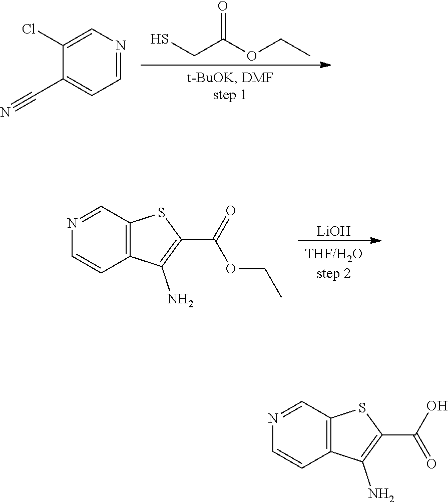

### Step 1: ethyl 3-aminothieno[2,3-c]pyridine-2-carboxylate

To a stirring solution of 3-chloroisonicotinonitrile (1 g, 7.2 mmol) and ethyl 2-mercaptoacetate (1.04 g, 8.7 mmol) in DMF (15 mL) was added t-BuOK (4.06 g, 36.2 mmol) at 0° C. under N₂. The resulting mixture was stirred at r.t. overnight, and slowly poured into H₂O (40 mL). The precipitate was collected by filtration and dried under vacuum to afford the title compound (200 mg, 12%). LCMS calc. for C₁₀H₁₁N₂O₂S [M+H]⁺: m/z=223.1; Found: 223.0.

### Step 2: 3-aminothieno[2,3-c]pyridine-2-carboxylic acid

A mixture of ethyl 3-aminothieno[2,3-c]pyridine-2-carboxylate (50 mg, 23.26 mmol) and LiOH (16.2 mg, 0.68 mmol) in THF (3 mL) and H₂O (3 mL) was stirred at 70° C. under N₂ for 3 h. The reaction mixture was concentrated in vacuo. The residue was diluted with H₂O (10 mL), adjusted to PH˜7 with citric acid. The mixture was extracted with EtOAc (30 mL×3). The combined organic layers were dried over anhydrous Na₂SO₄, filtered and concentrated under reduced pressure to afford the title compound (38 mg, 86%) as a yellow liquid. LCMS calc. for C₈H₇N₂O₂S [M+H]⁺: m/z=195.2; Found: 195.2.

### INT C4: 5-(5-Fluoro-2-methoxypyridin-4-yl)-1-((2-(trimethylsilyl)ethoxy)methyl)-1H-pyrazole-3-carboxylic acid

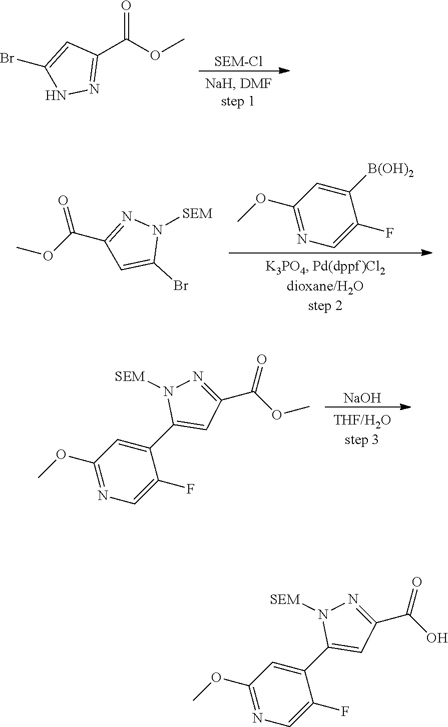

### Step 1: methyl 5-bromo-1-((2-(trimethylsilyl)ethoxy)methyl)-1H-pyrazole-3-carboxylate

To a solution of methyl 5-bromo-1H-pyrazole-3-carboxylate (5.0 g, 24.39 mmol) in DMF (80 mL) was added NaH (1.1 g, 26.8 mmol, 60% dispension in mineral oil) at 0° C. The mixture was stirred at 0° C. for 30 min., and SEM-Cl (6.1 g, 36.6 mmol) was added. The resulting mixture was stirred at r.t. for 3 h., then poured into water (200 mL) and extracted with EtOAc (100 mL×3). The combined organic layers were dried over Na₂SO₄, filtered and concentrated under reduced pressure. The residue was purified by flash chromatography on a silica gel column eluting with EtOAc/PE (0-100%) to afford the title compound (5.6 g, 68% yield) as a white solid. ¹H NMR (400 MHZ, DMSO-d₆) δ ppm 7.12 (s, 1H), 5.71 (s, 2H), 3.84 (s, 3H), 3.55 (dd, J=8.2, 7.5 Hz, 2H), 0.79 (dd, J=8.3, 7.5 Hz, 2H), −0.08 (s, 9H).

### Step 2: methyl 5-(5-fluoro-2-methoxypyridin-4-yl)-1-((2-(trimethylsilyl)ethoxy)methyl)-1H-pyrazole-3-carboxylate

A mixture of methyl 5-bromo-1-((2-(trimethylsilyl)ethoxy)methyl)-1H-pyrazole-3-carboxylate (4.8 g, 14.3 mmol), (5-fluoro-2-methoxypyridin-4-yl) boronic acid (3.7 g, 21.5 mmol), K₃PO₄ (9.1 g, 43.0 mmol) and Pd(dppf)Cl₂ (2.1 g, 2.9 mmol) in dioxane/H₂O (40 mL/8 mL) was degassed and recharged with nitrogen for three cycles. The mixture was stirred at 90° C. under N₂ overnight. The mixture was poured into water (100 mL) and extracted with EtOAc (50 mL×3). The combined organic layers were dried over anhydrous Na₂SO₄, filtered and concentrated under reduced pressure.

The residue was purified by flash chromatography on a silica gel column eluting with EtOAc/PE (0-60%) to afford the title compound (3.0 g, 55% yield) as a white solid. ¹H NMR (400 MHZ, DMSO-d₆) δ ppm 8.28 (d, J=2.5 Hz, 1H), 7.40 (d, J=3.1 Hz, 1H), 7.27 (d, J=5.0 Hz, 1H), 5.84 (s, 2H), 3.87 (d, J=4.6 Hz, 6H), 3.66-3.57 (m, 2H), 0.82 (dd, J=8.4, 7.5 Hz, 2H), −0.09 (s, 9H).

### Step 3. 5-(5-fluoro-2-methoxypyridin-4-yl)-1-((2-(trimethylsilyl)ethoxy)methyl)-1H-pyrazole-3-carboxylic acid

A mixture of methyl 5-(5-fluoro-2-methoxypyridin-4-yl)-1-((2-(trimethylsilyl)ethoxy)methyl)-1H-pyrazole-3-carboxylate (200 mg, 0.52 mmol) and NaOH (105 mg, 2.62 mmol) in THF (2 mL) and water (2 mL) was stirred at r.t. under N₂ overnight. The mixture was adjusted to pH=6 with HCl aq. (1 N) and extracted with EtOAc (30 mL×3). The combined organic layers were dried over anhydrous Na₂SO₄, filtered and concentrated under reduced pressure. The residue was purified by Prep-TLC eluting with MeOH/DCM (5%, with 0.1% FA) to afford the title compound (115 mg, 60% yield) as a white solid. ¹H NMR (400 MHZ, DMSO-d₆) δ ppm 13.83 (s, 1H), 8.28 (t, J=2.0 Hz, 1H), 7.33 (dd, J=3.4, 1.5 Hz, 1H), 7.27 (dd, J=5.1, 1.4 Hz, 1H), 5.85 (d, J=1.5 Hz, 2H), 3.87 (d, J=1.6 Hz, 3H), 3.69-3.57 (m, 2H), 0.86-0.79 (m, 2H), −0.05-−0.11 (m, 9H).

### INT C5: 1-Methyl-1H-pyrazole-4-sulfonamide

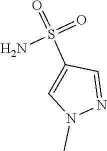

To a stirring solution of 1-methyl-1H-pyrazole-4-sulfonyl chloride (500 mg, 2.78 mmol) in DCM (15 mL) was added methanolic solution of ammonia (7 N) (3.5 mL) under N₂ atmosphere. The resulting mixture was stirred at r.t. for 14 h., and concentrated under reduced pressure to afford the title compound (440 mg, crude) as a white solid. LCMS calc. for C₄H₈N₃O₂S [M+H]⁺: m/z=162.0; Found: 162.2.

### INT C6: 4-Hydroxy-1H-pyrrolo[2,3-b]pyridine-5-carboxylic acid

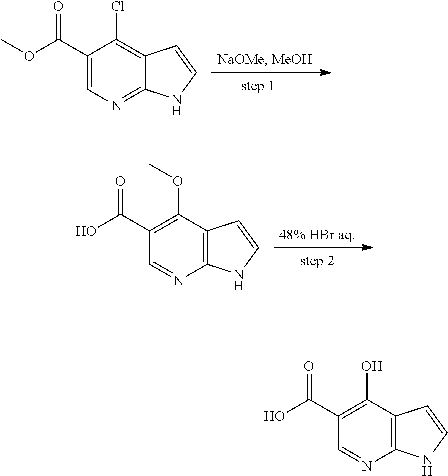

### Step 1: 4-methoxy-1H-pyrrolo[2,3-b]pyridine-5-carboxylic acid

To a solution of methyl 4-chloro-1H-pyrrolo[2,3-b]pyridine-5-carboxylate (500 mg, 2.37 mmol) in MeOH (5 mL) was added NaOMe (641 mg, 11.85 mmol). The mixture was stirred at 80° C. for 12 h. Saturated NH₄Cl aq. (20 mL) and water (10 mL) were added to reaction mixture. The mixture was extracted with EtOAc (20 mL×2). The combined organic layers were washed with water (20 mL) and brine (20 mL), dried over anhydrous Na₂SO₄, filtered and concentrated. The residue was purified by flash chromatography on a C18 column eluting with MeOH/H₂O (0-20%) to afford the title compound (240 mg, 49% yield) as a white solid. LCMS calc. for C₉H₉N₂O₃ [M+H]⁺: m/z=193.1; Found: 193.2.

### Step 2: 4-hydroxy-1H-pyrrolo[2,3-b]pyridine-5-carboxylic acid

A solution of 4-methoxy-1H-pyrrolo[2,3-b]pyridine-5-carboxylic acid (230 mg, 1.12 mmol) in HBr aq. (48%) (5 mL) was stirred at 140° C. for 2 h. The mixture was concentrated and the residue was purified by Prep-HPLC on a C18 column eluting with MeOH/H₂O (12%, with 0.1% FA) to afford the title compound (150 mg, 75% yield) as a yellow solid. LCMS calc. for C₈H₇N₂O₃ [M+H]⁺: m/z=179.0; Found: 179.2.

### INT C7: 7-Hydroxy-[1,2,4]triazolo[1,5-a]pyrimidine-6-carboxylic acid

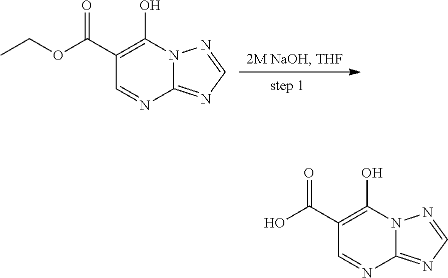

To a solution of ethyl 7-hydroxy-[1,2,4]triazolo[1,5-a]pyrimidine-6-carboxylate (100 mg, 0.48 mmol) in THF (2 mL) was added NaOH aq. (2 N, 2 mL). The mixture was stirred at r.t. overnight. The mixture was adjusted to pH=3 with HCl aq. (1 N), and concentrated. The residue was purified by Prep-HPLC on a C18 column eluting with MeCN/H₂O (0-2%) to afford the title compound (40 mg, 46% yield) as a white solid. LCMS calc. for C₆H₅N₄O₃ [M+H]⁺: m/z=181.0; Found: 181.2.

### INT C8: 4-Hydroxythieno[2,3-b]pyridine-5-carboxylic acid

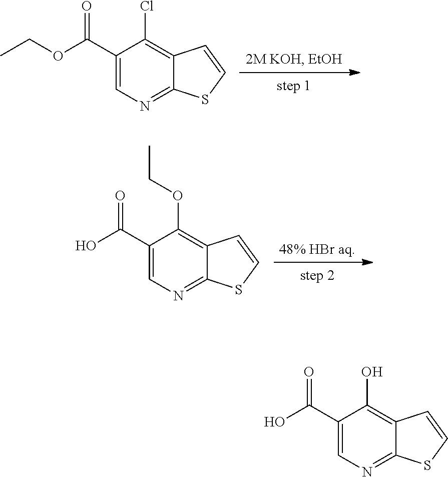

### Step 1: 4-ethoxythieno[2,3-b]pyridine-5-carboxylic acid

To a solution of KOH (174 mg, 3.1 mmol) in EtOH (4 mL) was added ethyl 4-chlorothieno[2,3-b]pyridine-5-carboxylate (150 mg, 0.6 mmol) at r.t. The mixture was stirred at 80° C. for 16 h. and concentrated. The residue was purified by Prep-HPLC on a C18 column eluting with MeCN/H₂O (15-55%, with 0.1% FA) to afford the title compound (70 mg, 50% yield) as a white solid. LCMS calc. for C₁₀H₁₀NO₃S [M+H]⁺: m/z=224.0; Found: 224.2.

### Step 2: 4-hydroxythieno[2,3-b]pyridine-5-carboxylic acid

A solution of 4-ethoxythieno[2,3-b]pyridine-5-carboxylic acid (60 mg, 0.268 mmol) in HBr aq. (4 mL, 48%) was stirred at 140° C. for 2 h. The reaction mixture was concentrated under reduced pressure. The residue was purified by Prep-HPLC on a C18 column eluting with MeOH/H₂O (12%, with 0.1% NH₄HCO₃) to afford the title compound (40 mg, 76% yield) as a white solid. LCMS calc. for C₈H₆NO₃S [M+H]⁺: m/z=196.0; Found: 196.1.

### INT C9: 1-(4-Methoxybenzyl)-4-(trifluoromethyl)-1H-pyrazole-5-carboxylic acid

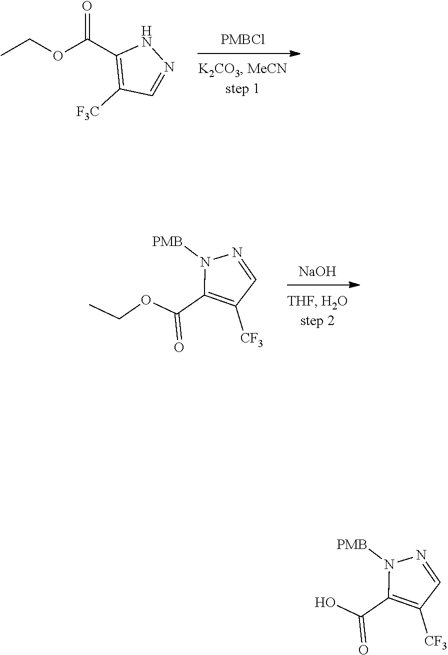

### Step 1: Ethyl 1-(4-methoxybenzyl)-4-(trifluoromethyl)-1H-pyrazole-5-carboxylate

To a solution of ethyl 4-(trifluoromethyl)-1H-pyrazole-5-carboxylate (250 mg, 1.20 mmol) in MeCN (5 mL) was added K₂CO₃ (200 mg, 1.44 mmol) and PMBCl (207 mg, 1.32 mmol). The mixture was stirred at r.t. overnight, diluted with water (30 mL) and extracted EtOAc (10 mL×3). The combined organic layers were dried over anhydrous Na₂SO₄, filtered and concentrated under reduced pressure. The residue was purified by flash chromatography on a silica gel column eluting with EtOAc/PE (20%) to afford the title compound (220 mg, 56% yield) as yellow oil. ¹H NMR (400 MHZ, CDCl₃) δ ppm 7.56 (s, 1H), 7.25 (d, J=1.9 Hz, 1H), 7.23 (d, J=1.9 Hz, 1H), 6.92 (d, J=8.6 Hz, 2H), 5.31 (s, 2H), 4.57-4.34 (m, 2H), 3.95-3.72 (m, 3H), 1.40 (t, J=7.1 Hz, 3H). LCMS calc. for C₁₅H₁₅F₃N₂O₃Na [M+Na]⁺: m/z=351.1; Found: 350.9.

### Step 2: 1-(4-methoxybenzyl)-4-(trifluoromethyl)-1H-pyrazole-5-carboxylic acid

To a solution of ethyl 1-(4-methoxybenzyl)-4-(trifluoromethyl)-1H-pyrazole-5-carboxylate (220 mg, 0.65 mmol) in THF (2 mL) and water (1 mL) was added NaOH (52 mg, 1.3 mmol). The mixture was stirred at r.t. for 2 h. The mixture was adjusted to pH=6 with HCl aq. (1 N). The mixture was concentrated under reduced pressure. The residue was purified by Prep-TLC eluting with MeOH/DCM (5%, with 0.1% FA) to afford the title compound (150 mg, 77% yield) as a white solid. LCMS calc. for C₁₃H₁₁F₃N₂O₃Na [M+Na]⁺: m/z=323.1; Found: 322.9.

### INT C10: 5-hydroxy-3-methyl-1,2,4-triazine-6-carboxylic acid

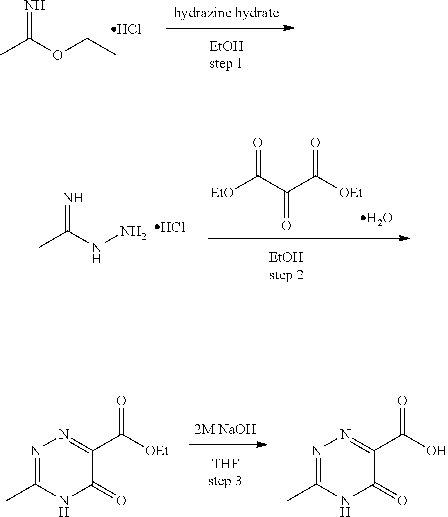

### Step 1: Acetimidohydrazide hydrochloride

To a solution of ethyl acetimidate hydrochloride (5.0 g, 40.5 mmol) in EtOH (60 mL) was added hydrazine hydrate (80% aqueous solution, 2.5 g, 40.5 mmol) dropwise at −78° C. The mixture was stirred at −78° C. for 1 h. The mixture was concentrated under reduced pressure. A small amount of EtOH (1 mL) was added to the precipitated crystals. The product was collected by filtration, washed with a small amount of EtOH (1 mL) and ethyl ether (2 mL) and dried to afford the title compound (2.5 g, 56% yield) as a white solid.

### Step 2: ethyl 5-hydroxy-3-methyl-1,2,4-triazine-6-carboxylate

To a solution of acetimidohydrazide hydrochloride (1.0 g, 9.13 mmol) in EtOH (20 mL) was added diethyl 2-oxomalonate hydrate (1.75 g, 9.1 mmol). The mixture was stirred at 80° C. for 4 h., and concentrated under reduced pressure. The residue was purified by flash chromatography on a silica gel column eluting with EtOAc/PE (0-30%) to afford the title compound (260 mg, 16% yield) as a white solid. LCMS calc. for C₇H₁₀N₃O₃ [M+H]⁺: m/z=184.1; Found: 184.0.

### Step 3: 5-hydroxy-3-methyl-1,2,4-triazine-6-carboxylic acid

To a solution of ethyl 5-hydroxy-3-methyl-1,2,4-triazine-6-carboxylate (220 mg, 1.20 mmol) in THF (2 mL) was added NaOH aq. (2 N, 1 mL). The mixture was stirred at r.t. overnight. The solvents were removed and water (2 mL) was added. The mixture was adjusted to pH=4 with HCl aq. (2 N) at 0° C. The mixture was extracted with EtOAc (5 mL×3). The combined organic layers were dried over anhydrous Na₂SO₄, filtered and concentrated under reduced pressure to afford the title compound (110 mg, 59% yield) as a white solid. LCMS calc. for C₅H₆N₃O₃ [M+H]⁺: m/z=156.1; Found: 156.1.

### INT C11: 3-Fluoro-N-hydroxypicolinimidoyl chloride

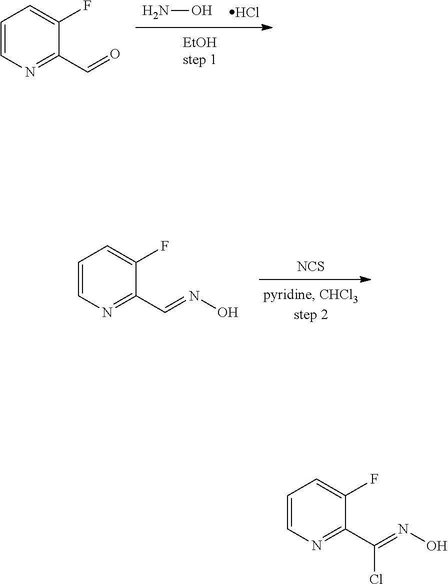

### Step 1: 3-Fluoropicolinaldehyde Oxime

To a solution of 3-fluoropyridine-2-carbaldehyde (1.0 g, 8 mmol) in EtOH (12 mL) was added hydroxylamine hydrochloride (0.84 g, 12 mmol) followed by sodium acetate (1.32 g, 16 mmol). The reaction was stirred for 30 min. at r.t. and partitioned between EtOAc (100 mL) and water (30 mL). The organic phase was separated and dried over MgSO₄, filtered and concentrated under vacuum to provide the title compound (0.46 g) which was used directly in the next step. LCMS calc. for C₆H₆FN₂O [M+H]⁺: m/z=141.0; Found: 141.1.

### Step 2: 3-fluoro-N-hydroxypicolinimidoyl chloride

To a suspension of 3-fluoropicolinaldehyde oxime (200 mg, 1.15 mmol) in CHCl₃ (2 mL) was added pyridine (10 mg, 0.12 mmol). The mixture was stirred at 40° C. and N-chlorosuccinimide (206 mg, 1.54 mmol) was added. The mixture was then stirred at 40° C. for 3 h., diluted with dichloromethane (50 mL), and washed with water (30 mL×3). The organic layer was concentrated under reduced pressure to yield the title compound (200 mg) as a yellow solid which was used directly in the next step.

### INT C12: 3,5-Dioxo-2,4-bis((2-(trimethylsilyl)ethoxy)methyl)-2,3,4,5-tetrahydro-1,2,4-triazine-6-carboxylic acid

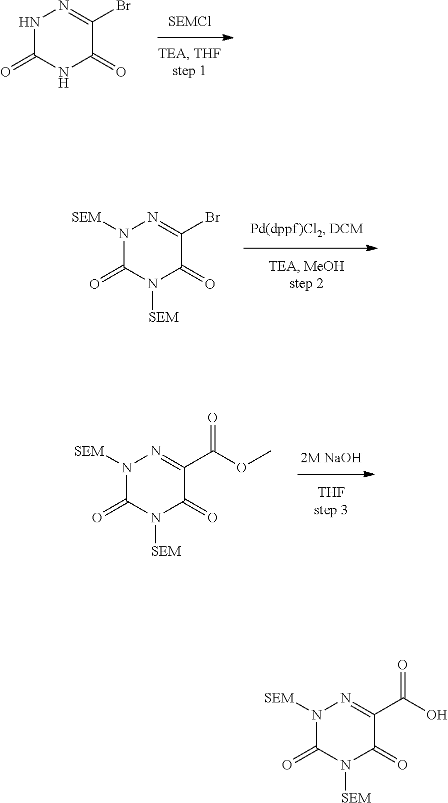 [SMILES: O=c1[nH]nc(Br)c(=O)[nH]1 ; "SEMCl" ; C[Si](C)(C)CCOCn1nc(Br)c(=O)n(COCC[Si](C)(C)C)c1=O ; COC(=O)c1nn(COCC[Si](C)(C)C)c(=O)n(COCC[Si](C)(C)C)c1=O ; C[Si](C)(C)CCOCn1nc(C(=O)O)c(=O)n(COCC[Si](C)(C)C)c1=O]

### Step 1: 6-bromo-2,4-bis((2-(trimethylsilyl)ethoxy)methyl)-1,2,4-triazine-3,5(2H,4H)-dione

To a mixture of 6-bromo-1,2,4-triazine-3,5(2H,4H)-dione (100 mg, 0.52 mmol) in THF (1 mL) was added TEA (0.174 mL, 1.25 mmol) and SEMCl (0.144 mL, 1.25 mmol) at 0° C. The mixture was stirred at 25° C. overnight and concentrated under reduced pressure. The residue was purified by Prep-TLC eluting with EtOAc/PE (20%) to afford the title compound (70 mg, 29% yield) as a white solid. ¹H NMR (400 MHZ, DMSO-d₆) δ ppm 5.21 (d, J=7.1 Hz, 4H), 3.70-3.57 (m, 4H), 0.94-0.81 (m, 4H), −0.02 (d, J=5.7 Hz, 18H).

### Step 2: methyl 3,5-dioxo-2,4-bis((2-(trimethylsilyl)ethoxy)methyl)-2,3,4,5-tetrahydro-1,2,4-triazine-6-carboxylate

To a solution of 6-bromo-2,4-bis((2-(trimethylsilyl)ethoxy)methyl)-1,2,4-triazine-3,5(2H,4H)-dione (1.6 g, 3.54 mmol) in MeOH (20 mL) were added Pd(dppf)Cl₂·DCM (286.6 mg, 0.35 mmol) and TEA (1.5 mL, 10.6 mmol). The mixture was stirred at 100° C. for 16 h. under CO atmosphere (40 bar). The resulting mixture was concentrated in vacuo. The residue was purified by flash chromatography on a silica gel column eluting with EtOAc/PE (20%) to afford the title compound (1 g, 65% yield) as a yellow solid. ¹H NMR (400 MHZ, DMSO-d₆) δ ppm 5.23 (d, J=19.9 Hz, 4H), 3.84 (s, 3H), 3.70-3.57 (m, 4H), 0.87 (td, J=8.9, 7.6 Hz, 4H), −0.02 (d, J=3.0 Hz, 18H).

### Step 3: 3,5-dioxo-2,4-bis((2-(trimethylsilyl)ethoxy)methyl)-2,3,4,5-tetrahydro-1,2,4-triazine-6-carboxylic acid

A mixture of methyl 3,5-dioxo-2,4-bis((2-(trimethylsilyl)ethoxy)methyl)-2,3,4,5-tetrahydro-1,2,4-triazine-6-carboxylate (100 mg, 0.23 mmol) in NaOH aq. (2 N, 0.23 mL) and THF (1 mL) was stirred at 80° C. for 2 h. The reaction mixture was adjusted to about pH=7 with HCl aq. (2 N), and concentrated under reduced pressure. The residue was triturated with MeCN, filtered and washed with MeCN (10 mL) to afford the title compound (50 mg, 52% yield) as a yellow solid. ¹H NMR (400 MHz, DMSO-d₆) δ ppm 5.15 (d, J=28.1 Hz, 4H), 3.72-3.52 (m, 4H), 0.96-0.78 (m, 4H), −0.03 (d, J=3.1 Hz, 18H).

### INT C13: 7-Chloro-5H-pyrrolo[3,2-d]pyrimidine-4-carboxylic acid

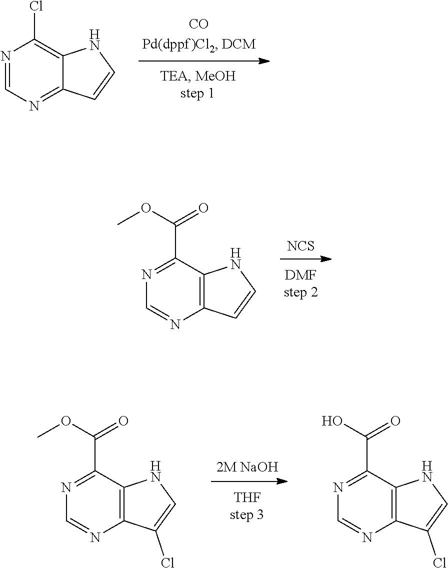

### Step 1: methyl 5H-pyrrolo[3,2-d]pyrimidine-4-carboxylate

A mixture of 4-chloro-5H-pyrrolo[3,2-d]pyrimidine (1.0 g, 6.5 mmol), TEA (2.0 g, 19.5 mmol) and Pd(dppf)Cl₂: DCM (531 mg, 0.65 mmol) in MeOH (20 mL) was stirred in autoclave at 100° C. under CO atmosphere overnight. After it was concentrated, the residue was poured into water (50 mL) and extracted with EtOAc (100 mL×3). The combined organic layers were washed with brine (50 mL), dried over Na₂SO₄, filtered and concentrated under reduced pressure. The residue was purified by flash chromatography on a silica gel column eluting with MeOH/DCM (0-8%) to give the title compound (870 mg, 75%) as a yellow solid. LCMS calc. for C₈H₈N₃O₂ [M+H]⁺: m/z=178.0; Found: 178.0.

### Step 2: methyl 7-chloro-5H-pyrrolo[3,2-d]pyrimidine-4-carboxylate

A mixture of methyl 5H-pyrrolo[3,2-d]pyrimidine-4-carboxylate (400 mg, 2.26 mmol) and NCS (453 mg, 3.39 mmol) in DMF (10 mL) was stirred at 50° C. for 8 h. under N₂ atmosphere. The reaction mixture was poured into water (50 mL) and extracted with EtOAc (80 mL×3). The combined organic layers were washed with brine (30 mL), dried over anhydrous Na₂SO₄, filtered and concentrated under reduced pressure. The residue was purified by Prep-TLC eluting with MeOH/DCM (5%) to give the title compound (220 mg, 46%) as a yellow solid. LCMS calc. for C₈H₇ClN₃O₂ [M+H]⁺: m/z=212.1; Found: 212.0.

### Step 3: 7-chloro-5H-pyrrolo[3,2-d]pyrimidine-4-carboxylic acid

A mixture of methyl 7-chloro-5H-pyrrolo[3,2-d]pyrimidine-4-carboxylate (60 mg, 0.28 mmol) and NaOH aq. (2 N, 1.4 mL) in THF (5 mL) was stirred at r.t. overnight. The mixture was concentrated under reduced pressure. The residue was adjusted to pH=5 to 6 with HCl aq. (1 N). Solids were collected by filtration to give the title compound (40 mg, 71%) as a white solid. LCMS calc. for C₇H₅ClN₃O₂ [M+H]⁺: m/z=198.0; Found: 198.0.

### INT C14: 1-(4-Methoxybenzyl)-4-(trifluoromethyl)-1H-imidazole-5-carboxylic acid

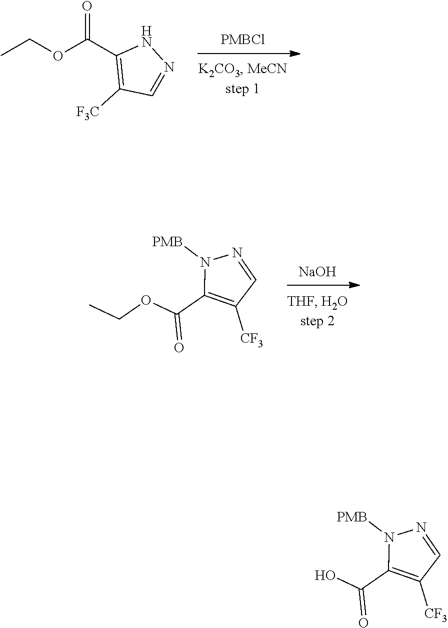

### Step 1: ethyl 1-(4-methoxybenzyl)-4-(trifluoromethyl)-1H-imidazole-5-carboxylate

A mixture of ethyl 4-(trifluoromethyl)-1H-imidazole-5-carboxylate (800 mg, 3.85 mmol), K₂CO₃ (638 mg, 4.62 mmol) and PMBCl (666 mg, 4.24 mmol) in MeCN (10 mL) was stirred at r.t. overnight. The reaction mixture was diluted with water (30 mL), and extracted with EtOAc (10 mL×3). The combined organic layers were dried over anhydrous Na₂SO₄, filtered and concentrated under reduced pressure. The residue was purified by flash chromatography on a silica gel column eluting with EtOAc/PE (20%) to afford the title compound (850 mg, 67% yield) as yellow oil. LCMS calc. for C₁₅H₁₆F₃N₂O₃ [M+H]⁺: m/z=329.1; Found: 329.0.

### Step 2: 1-(4-methoxybenzyl)-4-(trifluoromethyl)-1H-imidazole-5-carboxylic acid

To a solution of ethyl 1-(4-methoxybenzyl)-4-(trifluoromethyl)-1H-imidazole-5-carboxylate (350 mg, 1.07 mmol) in THF (2 mL) and water (1 mL) was added NaOH (85 mg, 2.14 mmol). The mixture was stirred at r.t. for 2 h. The mixture was adjusted to pH=6 with HCl aq. (1 N) and concentrated under reduced pressure. The residue was purified by Prep-TLC eluting with MeOH/DCM (5%, with 0.1% FA) to afford the title compound (300 mg, 93% yield) as a white solid. LCMS calc. for C₁₃H₁₁F₃N₂O₃Na [M+Na]⁺: m/z=323.1; Found: 322.9.

### INT C15: 4-(Trifluoromethyl)-1-((2-(trimethylsilyl)ethoxy)methyl)-1H-imidazole-5-carboxylic acid

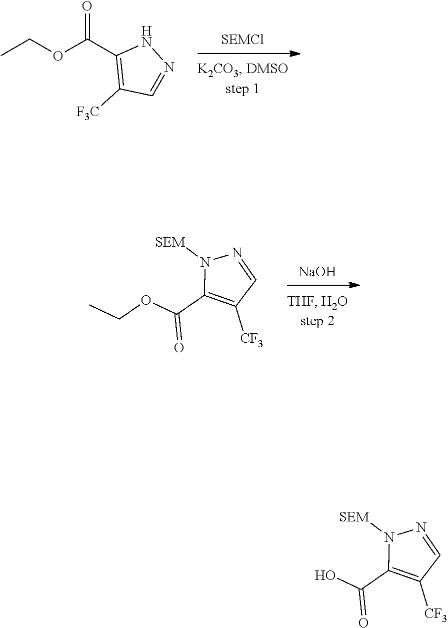

### Step 1: ethyl 4-(trifluoromethyl)-1-((2-(trimethylsilyl)ethoxy)methyl)-1H-imidazole-5-carboxylate

To a solution of ethyl 4-(trifluoromethyl)-1H-imidazole-5-carboxylate (300 mg, 1.44 mmol) in DMSO (5 mL) was added K₂CO₃ (600 mg, 4.32 mmol) and SEMCl (480 mg, 2.88 mmol). The mixture was stirred at r.t. overnight, diluted with water (30 mL) and extracted with EtOAc (10 mL×3). The combined organic layers were dried over anhydrous Na₂SO₄, filtered and concentrated under reduced pressure. The residue was purified by flash chromatography on a silica gel column eluting with EtOAc/PE (20%) to afford the title compound (400 mg, 82% yield) as a yellow solid. LCMS calc. for C₁₃H₂₂F₃N₂O₃Si [M+H]⁺: m/z=351.1; Found: 281.0.

### Step 2: 4-(trifluoromethyl)-1-((2-(trimethylsilyl)ethoxy)methyl)-1H-imidazole-5-carboxylic acid

To a solution of ethyl 4-(trifluoromethyl)-1-((2-(trimethylsilyl)ethoxy)methyl)-1H-imidazole-5-carboxylate (400 mg, 1.18 mmol) in THF (5 mL) was added NaOH (95 mg, 2.36 mmol) in water (1 mL). The mixture was stirred at 50° C. for 2 h. and adjusted to pH=5˜ 6 with HCl aq. (1 N) and filtered. The filtrate was concentrated to afford the title compound (300 mg, 82%) as a yellow solid. LCMS calc. for C₁₁H₁₈F₃N₂O₃Si [M+H]⁺: m/z=310.1; Found: 253.0.

### INT C16: 3-amino-5-fluoropicolinic acid

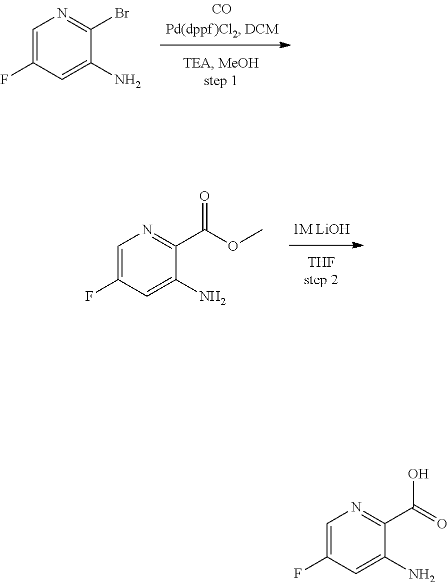

### Step 1: methyl 3-amino-5-fluoropicolinate

A mixture of 2-bromo-5-fluoropyridin-3-amine (1.0 g, 5.26 mmol), TEA (5.32 g, 52.63 mmol) and Pd(dppf)Cl₂ DCM (430 mg, 0.53 mmol) in MeOH (15 mL) was stirred in autoclave at 100° C. under CO atmosphere overnight. The mixture was concentrated under reduced pressure. The resulting residue was poured into water (50 mL), extracted with EtOAc (50 mL×3). The combined organic layers were washed with brine, dried over anhydrous Na₂SO₄, filtered and concentrated under reduced pressure. The residue was purified by flash chromatography on a silica gel column eluting with EtOAc/PE (0-50%) to afford the title compound (310 mg, 38%) as a yellow solid.

### Step 2: 3-amino-5-fluoropicolinic acid

To a solution of methyl 3-amino-5-fluoropicolinate (100 mg, 0.58 mmol) in THF (3 mL) was added LiOH aq. (1N, 1.5 mL). The mixture was stirred at r.t. for 3 h., adjusted to pH=4 with HCl aq. (1N) solution, and concentrated under reduced pressure to afford the title compound (70 mg) as a yellow solid. LCMS calc. for C₆H₆FN₂O₂ [M+H]⁺: m/z=157.0; Found: 157.2.

### INT C17: 7-hydroxythiazolo[5,4-b]pyridine-6-carboxylic acid

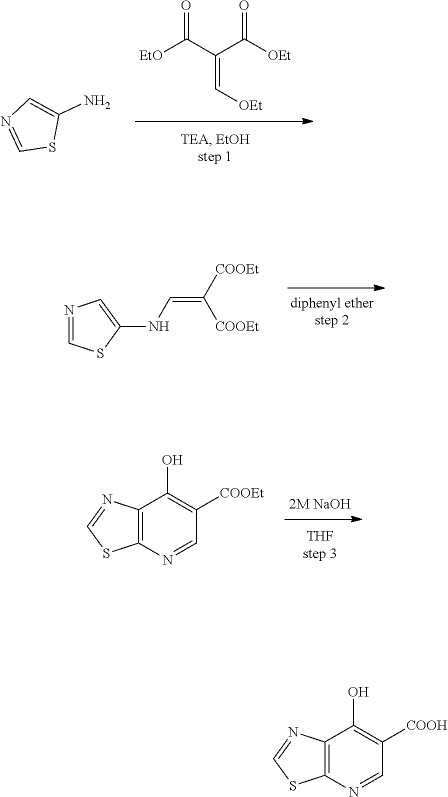 [SMILES: Nc1cncs1 ; CCOC=C(C(=O)OCC)C(=O)OCC ; CCOC(=O)C(=CNc1cncs1)C(=O)OCC ; CCOC(=O)c1cnc2scnc2c1O ; O=C(O)c1cnc2scnc2c1O]

### Step 1: diethyl 2-((thiazol-5-ylamino)methylene)malonate

To a solution of 5-aminothiazole HCl salt (500 mg, 3.66 mmol) in EtOH (20 mL) was added diethyl 2-(ethoxymethylene)malonate (791 mg, 3.66 mmol) and TEA (444 mg, 4.39 mmol). The mixture was stirred at 90° C. for 3 h. and concentrated under reduced pressure. The residue was purified by flash chromatography on a silica gel column eluting with EtOAc/PE (1-45%) to afford the title compound (530 mg, 54% yield) as a yellow solid. ¹H NMR (400 MHZ, DMSO-d₆) δ ppm 10.82 (d, J=13.4 Hz, 1H), 8.73 (d, J=0.9 Hz, 1H), 7.98 (d, J=13.5 Hz, 1H), 7.82 (d, J=0.9 Hz, 1H), 4.20 (q, J=7.1 Hz, 2H), 4.11 (q, J=7.1 Hz, 2H), 1.27-1.24 (m, 3H), 1.23-1.20 (m, 3H).

### Step 2: ethyl 7-hydroxythiazolo[5,4-b]pyridine-6-carboxylate

A solution of diethyl 2-((thiazol-5-ylamino)methylene)malonate (300 mg, 1.11 mmol) in diphenyl ether (2 mL) was stirred at 250° C. for 45 min. The mixture was cooled to r.t. and diluted with hexanes (20 mL). The mixture was then stirred at 0° C. for 1 h. The precipitate was collected by filtration and further purified by Prep-HPLC on a C18 column eluting with MeOH/H₂O (1%-42%) to give the title compound (130 mg, 52% yield) as a yellow solid. ¹H NMR (400 MHZ, DMSO-d₆) δ ppm 12.30 (s, 1H), 9.35 (s, 1H), 8.80 (s, 1H), 4.37 (q, J=7.1 Hz, 2H), 1.35 (t, J=7.1 Hz, 3H).

### Step 3: 7-hydroxythiazolo[5,4-b]pyridine-6-carboxylic acid

To a solution of ethyl 7-hydroxythiazolo[5,4-b]pyridine-6-carboxylate (50 mg, 0.22 mmol) in THF (2 mL) was added NaOH aq. (2 N, 1.5 mL). The mixture was stirred at 30° C. overnight. The mixture was adjusted to pH=4 with HCl aq. (1 N). The precipitate were collected by filtration to afford the title compound (30 mg, 68% yield) as a yellow solid. LCMS calc. for C₇H₅N₂O₃S [M+H]⁺: m/z=197.0; Found: 197.2.

### INT C18: 1-((2-(Trimethylsilyl)ethoxy)methyl)-1H-pyrazolo[4,3-d]pyrimidine-7-carboxylic acid

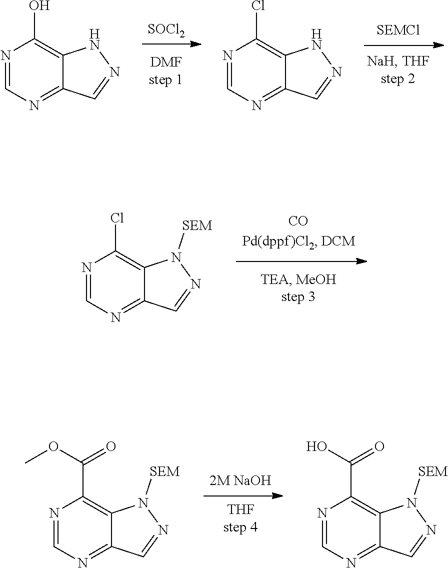

### Step 1: 7-chloro-1H-pyrazolo[4,3-d]pyrimidine

A mixture of 1H-pyrazolo[4,3-d]pyrimidin-7-ol (2.0 g, 14.7 mmol) and SOCl₂ (10 mL) in DMF (10 mL) was stirred at 90° C. for 1 h. The mixture was concentrated under reduced pressure. The residue was poured into water (50 mL), extracted with EtOAc (200 mL×3). The combined organic layers were washed with saturated NaHCO₃ aq. (100 mL) and brine (100 mL), dried over anhydrous Na₂SO₄, filtered and concentrated under reduced pressure to give the title compound (1.45 g, 64%) as a yellow solid. LCMS calc. for C₅H₄ClN₄ [M+H]⁺: m/z=155.0; Found: 155.0.

### Step 2: 7-chloro-1-((2-(trimethylsilyl)ethoxy)methyl)-1H-pyrazolo[4,3-d]pyrimidine

A mixture of 7-chloro-1H-pyrazolo[4,3-d]pyrimidine (1.2 g, 7.8 mmol), SEMCl (2.6 g, 15.6 mmol) and NaH (60% dispersion in mineral oil, 623 mg, 15.6 mmol) in THF (20 mL) was stirred at r.t overnight. The mixture was poured into water (50 mL), extracted with EtOAc (100 mL×3). The combined organic layers were washed with brine (30 mL), dried over anhydrous Na₂SO₄, filtered and concentrated under reduced pressure. The residue was purified by flash chromatography on a silica gel column eluting with EtOAc/PE (0-20%) to afford the title compound (700 mg, 32%) as a yellow solid. LCMS calc. for C₁₁H₁₈ClN₄OSi [M+H]⁺: m/z=285.1; Found: 285.0.

### Step 3: methyl 1-((2-(trimethylsilyl)ethoxy)methyl)-1H-pyrazolo[4,3-d]pyrimidine-7-carboxylate

A mixture of 7-chloro-1-((2-(trimethylsilyl)ethoxy)methyl)-1H-pyrazolo[4,3-d]pyrimidine (700 mg, 2.46 mmol), TEA (748 mg, 7.39 mmol) and PdCl₂(dppf)·DCM (201 mg, 0.25 mmol) in MeOH (25 mL) was stirred in autoclave at 100° C. under CO atmosphere overnight. The mixture was concentrated under reduced pressure. The residue was poured into water (50 mL), extracted with EtOAc (80 mL×3). The combined organic layers were washed with brine (30 mL), dried over anhydrous Na₂SO₄, filtered and concentrated under reduced pressure. The residue was purified by flash chromatography on a silica gel column eluting with EtOAc/PE (0-40%) to afford the title compound (520 mg, 68%) as a yellow solid. LCMS calc. for C₁₃H₂₁N₄O₃Si [M+H]⁺: m/z=309.1; Found: 309.2.

### Step 4: 1-((2-(trimethylsilyl)ethoxy)methyl)-1H-pyrazolo[4,3-d]pyrimidine-7-carboxylic acid

A mixture of methyl 1-((2-(trimethylsilyl)ethoxy)methyl)-1H-pyrazolo[4,3-d]pyrimidine-7-carboxylate (520 mg, 1.69 mmol) and NaOH aq. (2 N, 8.5 mL) in THF (10 mL). The mixture was stirred at r.t. overnight. The mixture was concentrated under reduced pressure. The residue was diluted with HCl aq. (1N) until weak acidity. The precipitate were collected by filtration to afford the title compound (310 mg, 62%) as a white solid. LCMS calc. for C₁₂H₁₇N₄O₃Si [M−H]⁺: m/z=293.2; Found: 293.2.

### INT C19: 5-(Ethoxycarbonyl)-4-hydroxyfuro[2,3-b]pyridine-2-carboxylic acid

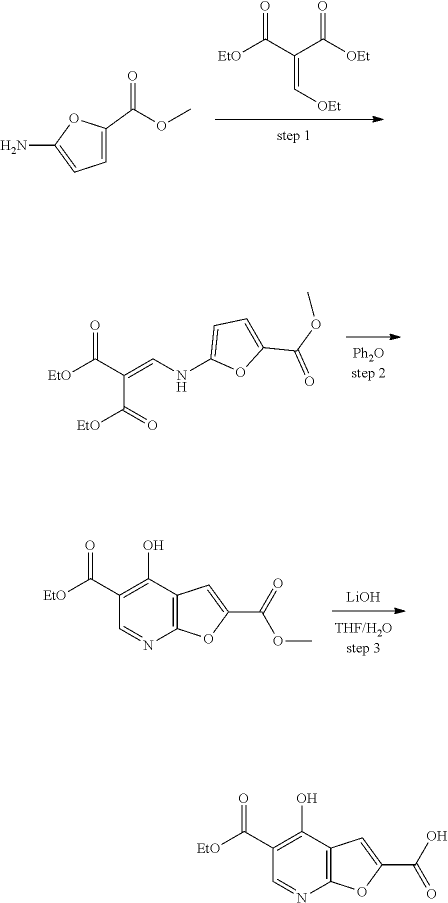

### Step 1: diethyl 2-(((5-(methoxycarbonyl)furan-2-yl)amino)methylene)malonate

A solution of methyl 5-aminofuran-2-carboxylate (1 g, 7.09 mmol) in diethyl 2-(ethoxymethylene)malonate (5 mL) was stirred at 120° C. for 4 h., and cooled to r.t. The cooled reaction mixture was diluted with 2-propanol (5 ml), seeded and stirred at r.t. overnight. The precipitate was isolated by filtration and dried under reduced pressure to afford the title compound (1.0 g, 45% yield) as a yellow solid. LCMS calc. for C₁₄H₁₈NO₇ [M+H]⁺: m/z=312.1. Found: 312.0.

### Step 2: 5-ethyl 2-methyl 4-hydroxyfuro[2,3-b]pyridine-2,5-dicarboxylate

A solution of diethyl 2-(((5-(methoxycarbonyl)furan-2-yl)amino)methylene)malonate (1 g, 3.21 mmol) in Ph₂O (5 mL) was stirred at 240° C. for 2 h. and cooled to r.t. The cooled reaction mixture was diluted with hexane (5 ml). The product was collected by filtration and dried in vacuo to obtain the title compound (460 mg, 54% yield) as a yellow solid. LCMS calc. for C₁₂H₁₂NO₆ [M+H]⁺: m/z=266.1; Found: 266.6.

### Step 3: 5-(ethoxycarbonyl)-4-hydroxyfuro[2,3-b]pyridine-2-carboxylic acid

A mixture of 5-ethyl 2-methyl 4-hydroxyfuro[2,3-b]pyridine-2,5-dicarboxylate (460 mg, 1.73 mmol) and LiOH (208 mg, 8.6 mmol) in THF/H₂O (1:1) (2 mL) was stirred at r.t. for 1 h. The mixture was adjusted to pH to 4-5 with HCl aq. (1 N) and concentrated. The residue was purified by Prep-HPLC on a C18 column eluting with MeCN/H₂O (10-95%, with 0.1% NH₄HCO₃) to afford the title compound (320 mg, 74% yield) as a white solid. LCMS calc. for C₁₁H₁₀NO₆ [M+H]⁺: m/z=252.0; Found: 252.1.

### INT C20: 4-methyl-3-oxo-3,4-dihydropyrazine-2-carboxylic acid

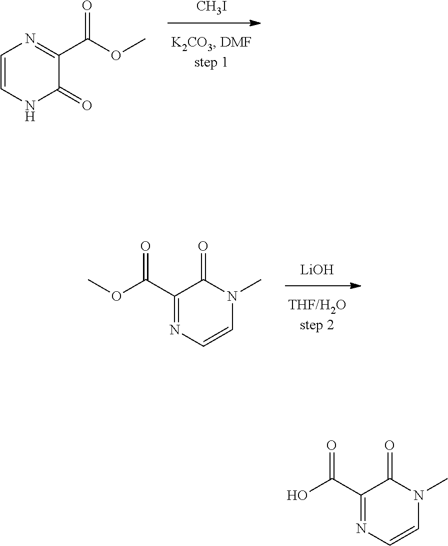

### Step 1: methyl 4-methyl-3-oxo-3,4-dihydropyrazine-2-carboxylate

To a solution of methyl 3-oxo-3,4-dihydropyrazine-2-carboxylate (500 mg, 3.25 mmol) in DMF (10 mL) was added K₂CO₃ (897 mg, 6.49 mmol) and CH₃I (691 mg, 4.87 mmol). The mixture was stirred at 50° C. for 4 h., concentrated and diluted with H₂O (100 mL). The resulting mixture was adjusted to pH to 4 with HCl aq. (1N) solution and extracted with DCM (100 mL×3). The organic layers were combined and concentrated under reduced pressure. The residue was purified by flash chromatography on a silica gel column eluting with EtOAc/PE (5%) to afford the title compound (150 mg, 28% yield) as a yellow solid. ¹H NMR (400 MHZ, CDCl₃) δ ppm 7.43 (d, J=4.1 Hz, 1H), 7.36 (d, J=4.1 Hz, 1H), 3.97 (s, 3H), 3.61 (s, 3H).

### Step 2: 4-methyl-3-oxo-3,4-dihydropyrazine-2-carboxylic acid

To a solution of methyl 4-methyl-3-oxo-3,4-dihydropyrazine-2-carboxylate (100 mg, 0.60 mmol) in MeOH (2 mL) and H₂O (0.5 mL) was added LiOH·H₂O (50 mg, 1.20 mmol). The mixture was stirred at r.t. for 2 h. The mixture was adjusted to pH to 4˜5 with HCl aq. (1 N) and then concentrated under reduced pressure. The residue was purified by Prep-HPLC on a C18 column eluting with MeCN/H₂O (20-40%, with 0.1% FA) to afford the title compound (60 mg, 65% yield) as a white solid. ¹H NMR (400 MHZ, DMSO-d₆) δ ppm 7.83 (s, 1H), 7.35 (d, J=4.0 Hz, 1H), 3.50 (s, 3H).

### INT C21: 7-((2-(Trimethylsilyl)ethoxy)methyl)-7H-purine-6-carboxylic acid

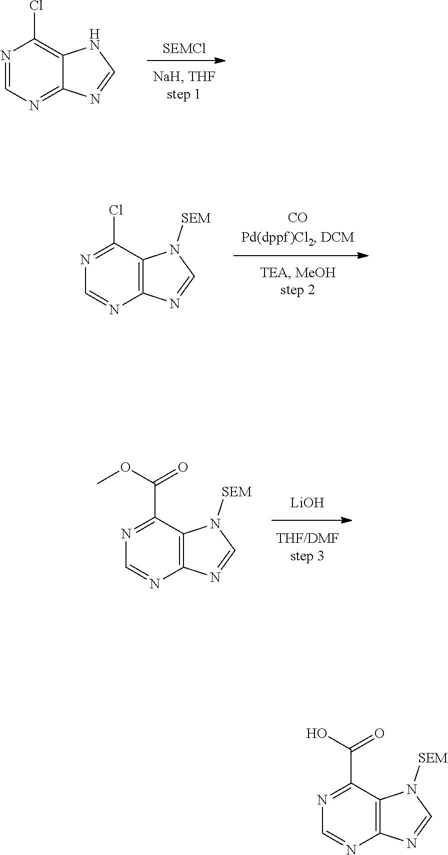 [SMILES: Clc1ncnc2nc[nH]c12 ; "SEMCl" ; C[Si](C)(C)CCOCn1cnc2ncnc(Cl)c21 ; COC(=O)c1ncnc2ncn(COCC[Si](C)(C)C)c12 ; "LiOH" ; C[Si](C)(C)CCOCn1cnc2ncnc(C(=O)O)c21]

### Step 1: 6-chloro-7-((2-(trimethylsilyl)ethoxy)methyl)-7H-purine

To a suspension of NaH (60% dispersion in mineral oil, 234 mg, 9.74 mmol) in DMF (5 mL) was added a solution of 6-chloro-7H-purine (1 g, 6.49 mmol) in DMF (5 mL) at 0° C. under N₂ atmosphere. After the mixture was stirred at 0° C. for 0.5 h., SEM-Cl (2.17 g, 13.0 mmol) was added. The resulting mixture was stirred at 0° C. for 3 h. and extracted with EtOAc (10 mL×3). The organic layers were separated and concentrated under reduced pressure. The residue was purified by flash chromatography on a silica gel column eluting with EtOAc/PE (0-20%) to afford the title compound (1.32 g, 72% yield) as a white solid. LCMS calc. for C₁₁H₁₈ClN₄OSi [M+H]⁺: m/z=285.1; Found: 285.1.

### Step 2: methyl 7-((2-(trimethylsilyl)ethoxy)methyl)-7H-purine-6-carboxylate

To a stirring solution of 6-chloro-7-((2-(trimethylsilyl)ethoxy)methyl)-7H-purine (500 mg, 1.76 mmol) in MeOH (10 mL) and DMF (3 mL) was added Pd(dppf)Cl₂ (138 mg, 0.19 mmol) and TEA (10 mL) at r.t. The resulting mixture was stirred at 100° C. for 16 h. in an autoclave under CO atmosphere. The reaction mixture was concentrated under reduced pressure. The residue was purified by Prep-HPLC on a C18 column eluting with MeOH/H₂O (10%-20%, with 0.1% NaHCO₃) to afford the title compound (155 mg, 29% yield) as a brown solid. LCMS calc. for C₁₃H₂₁N₄O₃Si [M+H]⁺: m/z=309.1; Found: 309.2.

### Step 3: 7-((2-(trimethylsilyl)ethoxy)methyl)-7H-purine-6-carboxylic acid

To a stirring solution of methyl 7-((2-(trimethylsilyl)ethoxy)methyl)-7H-purine-6-carboxylate (145 mg, 0.47 mmol) in THF (1 mL) and DMF (0.1 mL) was added LiOH (34 mg, 1.41 mmol) at 0° C. The resulting mixture was stirred at 0° C. for 0.5 h., adjusted to pH=4 with HCl aq. (2 N), and then extracted with EtOAc (5 mL×3). The combined organic layers were washed with brine (20 mL), dried over Na₂SO₄, filtered and concentrated under reduced pressure to afford the title compound (30 mg, 22% yield) as a white solid. LCMS calc. for C₁₂H₁₉N₄O₃Si [M+H]⁺: m/z=295.1; Found: 295.2.

### INT C22: 7-Hydroxy-3H-imidazo[4,5-b]pyridine-6-carboxylic acid

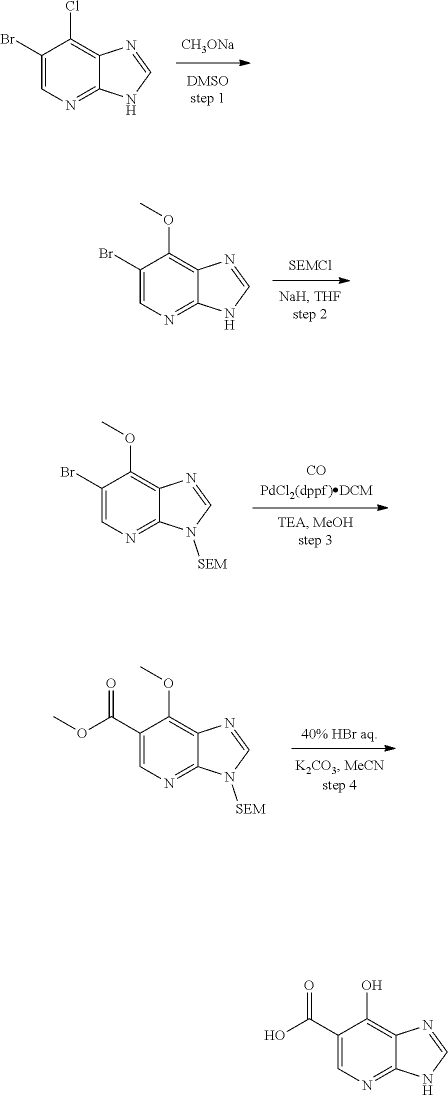

### Step 1: 6-bromo-7-methoxy-3H-imidazo[4,5-b]pyridine

A mixture of 6-bromo-7-chloro-3H-imidazo[4,5-b]pyridine (500 mg, 2.2 mmol) and CH₃ONa (1.16 g, 2.2 mmol) in DMSO (10 mL) was heated under microwave at 150° C. for 3 h. The mixture was poured into water (30 mL) and extracted with EtOAc (50 mL×3). The combined organic layers were washed with brine (20 mL), dried over Na₂SO₄, filtered, and concentrated under reduced pressure to afford the title compound (400 mg, 82% yield) as a yellow solid. LCMS calc. for C₇H₇BrN₃O [M+H]⁺: m/z=228.1; Found: 228.0.

### Step 2: 6-bromo-7-methoxy-3-((2-(trimethylsilyl)ethoxy)methyl)-3H-imidazo[4,5-b]pyridine

A mixture of 6-bromo-7-methoxy-3H-imidazo[4,5-b]pyridine (350 mg, 1.53 mmol), SEMCl (512 mg, 3.07 mmol) and NaH (60% dispersion in mineral oil, 123 mg, 3.07 mmol) in THF (10 mL) was stirred at r.t. overnight. The mixture was poured into water (30 mL), extracted with EtOAc (40 mL×3). The combined organic layers were washed with brine (20 mL), dried over Na₂SO₄, filtered, and concentrated under reduced pressure. The residue was purified by flash chromatography on a silica gel column eluting with EtOAc/PE (20%) to afford the title compound (220 mg, 40% yield) as a yellow oil. LCMS calc. for C₁₃H₂₁BrN₃O₂Si [M+H]⁺: m/z=358.0; Found: 358.0.

### Step 3: methyl 7-methoxy-3-((2-(trimethylsilyl)ethoxy)methyl)-3H-imidazo[4,5-b]pyridine-6-carboxylate

A mixture of 6-bromo-7-methoxy-3-((2-(trimethylsilyl)ethoxy)methyl)-3H-imidazo[4,5-b]pyridine (105 mg, 0.29 mmol), TEA (298 mg, 2.94 mmol) and PdCl₂(dppf)·DCM (72 mg, 0.09 mmol) in MeOH (10 mL) was stirred in an autoclave at 120° C. under CO for 45 h. The mixture was concentrated under reduced pressure, poured into water (30 mL), extracted with EtOAc (40 mL×3). The combined organic layers were washed with brine (20 mL), dried over Na₂SO₄, filtered, and concentrated under reduced pressure. The resulting residue was purified by flash chromatography on a silica gel column eluting with EtOAc/PE (30%) to afford the title compound (43 mg, 43.4% yield) as a yellow oil. LCMS calc. for C₁₅H₂₄N₃O₄Si [M+H]⁺: m/z=338.2; Found: 338.2.

### Step 4: 7-hydroxy-3H-imidazo[4,5-b]pyridine-6-carboxylic acid

A solution of methyl 7-methoxy-3-((2-(trimethylsilyl)ethoxy)methyl)-3H-imidazo[4,5-b]pyridine-6-carboxylate (43 mg, 0.13 mmol) in aq. HBr (40% solution, 2 mL) was stirred at 70° C. for 14 h. The mixture was concentrated to afford the title compound (18 mg, 79% yield) as a yellow solid. LCMS calc. for C₇H₄N₃O₃ [M−H]⁻: m/z=178.0; Found: 178.0.

### INT C23: 2-Methyl-3,5-dioxo-2,3,4,5-tetrahydro-1,2,4-triazine-6-carboxylic acid

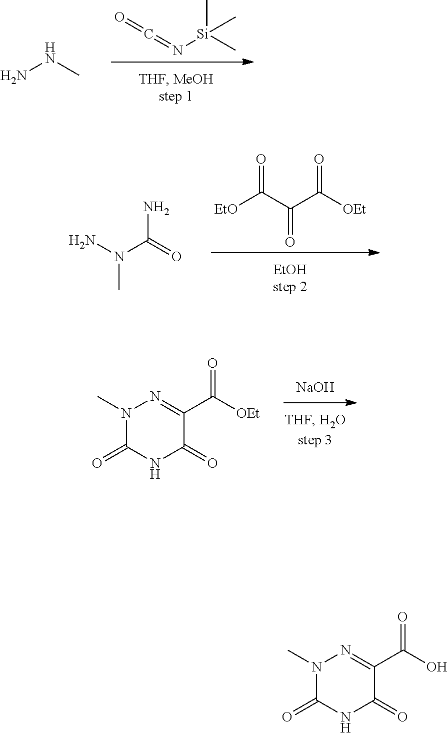

### Step 1: 1-methylhydrazine-1-carboxamide

To a solution of methylhydrazine (2.6 g, 56.5 mmol) in THF (15 mL) was added isocyanatotrimethylsilane (5 g, 43.5 mmol) at 0° C. The mixture was stirred at 0° C. for 1 h., and then MeOH (8 mL) was added. The resulting mixture was stirred at 40° C. for 5 h. and concentrated under reduced pressure to afford the title compound (2.5 g, 65% yield) as a light yellow solid. LCMS calc. for C₂H₈N₃O [M+H]⁺: m/z=90.1; Found: 90.2.

### Step 2: ethyl 2-methyl-3,5-dioxo-2,3,4,5-tetrahydro-1,2,4-triazine-6-carboxylate

To a solution of 1-methylhydrazine-1-carboxamide (500 mg, 5.6 mmol) in EtOH (15 mL) was added diethyl 2-oxomalonate (978 mg, 5.6 mmol). The mixture was stirred at 90° C. overnight, diluted with water (30 mL) and extracted with EtOAc (10 mL×3). The combined organic layers were dried over anhydrous Na₂SO₄, filtered and concentrated. The residue was purified by flash chromatography on a silica gel column eluting with MeOH/DCM (5%) to afford the title compound (300 mg, 27% yield) as a white solid. LCMS calc. for C₇H₁₀N₃O₄ [M+H]⁺: m/z=200.1; Found: 200.0.

### Step 3: 2-methyl-3,5-dioxo-2,3,4,5-tetrahydro-1,2,4-triazine-6-carboxylic acid

To a solution of ethyl 2-methyl-3,5-dioxo-2,3,4,5-tetrahydro-1,2,4-triazine-6-carboxylate (100 mg, 0.51 mmol) in THF (1 mL) was added NaOH (40 mg, 1.01 mmol) in H₂O (1 mL). The mixture was stirred at r.t. for 2 h. The mixture was adjusted to pH 5-6 with HCl aq. (1 N, 2 mL), filtered and dried to afford the title compound (60 mg, 70% yield) as a white solid. LCMS calc. for C₅H₆N₃O₄ [M+H]⁺: m/z=172.0; Found: 172.0.

### INT C24: 1-Methyl-2,4-dioxo-1,2,3,4-tetrahydropyrimidine-5-carboxylic acid

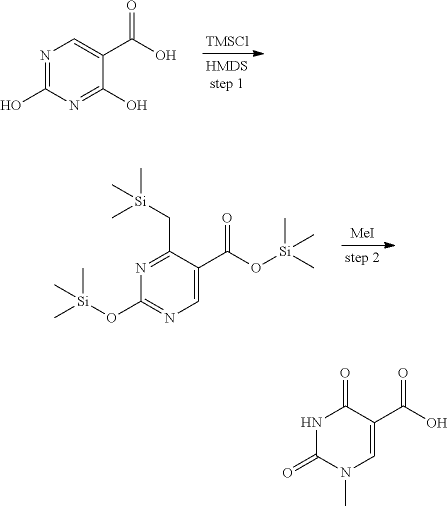

### Step 1: trimethylsilyl 2,4-bis((trimethylsilyl)oxy)pyrimidine-5-carboxylate

To a solution of 2,4-dihydroxypyrimidine-5-carboxylic acid (200 mg, 1.28 mmol) in HMDS (2 mL) was added chlorotrimethylsilane (TMSCl) (278 mg, 2.56 mmol). The mixture was stirred at 120° C. for 6 h. The reaction mixture was used for the next step without further purification.

### Step 2: 1-methyl-2,4-dioxo-1,2,3,4-tetrahydropyrimidine-5-carboxylic acid

To the above reaction mixture was added iodomethane (0.8 mL, 12.8 mmol). The resulting mixture was stirred at 50° C. overnight and concentrated under vacuum. The residue was purified by Prep-HPLC on a C18 column eluting with MeOH/H₂O (5-50%) to afford the title compound (46 mg, 21% yield) as a white solid. ¹H NMR (400 MHZ, DMSO-d₆) δ ppm 12.77 (s, 1H), 12.20 (s, 1H), 8.67 (s, 1H), 3.38 (s, 3H). LCMS calc. for C₆H₇N₂O₄ [M+H]⁺: m/z=171.0; Found: 171.0.

### INT C25: 3-Oxo-2,3-dihydro-1H-pyrazole-4-carboxylic acid

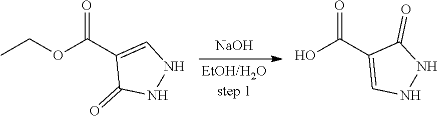

A mixture of ethyl 3-oxo-2,3-dihydro-1H-pyrazole-4-carboxylate (100 mg, 0.64 mmol) and NaOH (153.8 mg, 3.84 mmol) in EtOH/H₂O (1:1) (4 mL) was stirred at 80° C. for 2 days. The reaction mixture was diluted with H₂O (5 mL) and washed with EtOAc (5 mL×3). The aqueous layer was concentrated under reduced pressure to afford the title compound (80 mg, 97% yield) as a white solid. ¹H NMR (400 MHZ, DMSO-d₆) δ ppm 12.12 (s, 1H), 7.84 (s, 1H). LCMS calc. for C₄H₅N₂O₃ [M+H]⁺: m/z=129.1; Found: 129.3.

### INT C26: 1-Methyl-3-oxo-2,3-dihydro-1H-pyrazole-4-carboxylic acid

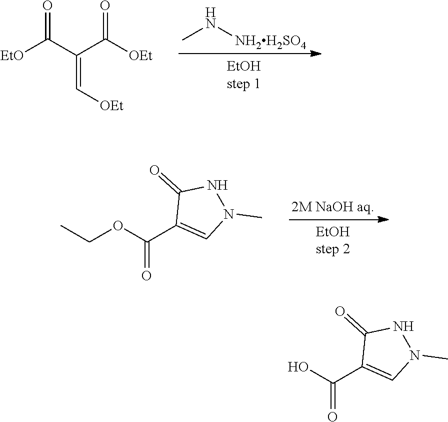

### Step 1: ethyl 1-methyl-3-oxo-2,3-dihydro-1H-pyrazole-4-carboxylate

Methylhydrazine sulfate (10 g, 69.4 mmol) in water (30 mL) was added to diethyl 2-(ethoxymethylene)malonate (4.5 g, 20.8 mmol) in ethanol (20 mL). The mixture was stirred at 100° C. for 1 h., and concentrated under reduced pressure. The crude product was recrystallized from ethanol. The precipitate was collected by filtration to afford the title compound (1.28 g, 36.2% yield) as a white solid. ¹HNMR (400 MHZ, CDCl₃) δ ppm 7.54 (s, 1H), 4.32 (q, J=7.2 Hz, 2H), 3.76 (s, 3H), 1.35 (t, J=7.2 Hz, 3H). LCMS calc. for C₇H₁₁N₂O₃ [M+H]⁺: m/z=171.1; Found: 171.2.

### Step 2: 1-methyl-3-oxo-2,3-dihydro-1H-pyrazole-4-carboxylic acid

A mixture of ethyl 1-methyl-3-oxo-2,3-dihydro-1H-pyrazole-4-carboxylate (100 mg, 0.12 mmol) and NaOH aq. (2 N, 0.7 mL) in EtOH (1.5 mL) was stirred at 65° C. for 4 h., and concentrated under reduced pressure. The residue was slowly neutralized with HCl aq. (1 N) to PH˜6. The precipitates were collected by filtration and dried under vacuum to afford the title compound (82 mg, 98.2%) as a white solid. ¹HNMR (400 MHZ, DMSO-d₆) δ ppm 7.90-7.86 (m, 1H), 3.63 (s, 3H). LCMS calc. for C₅H₇N₂O₃ [M+H]⁺: m/z=143.0; Found: 143.2.

### INT C27: 4-Hydroxy-1-methyl-1H-pyrrolo[2,3-b]pyridine-5-carboxylic acid

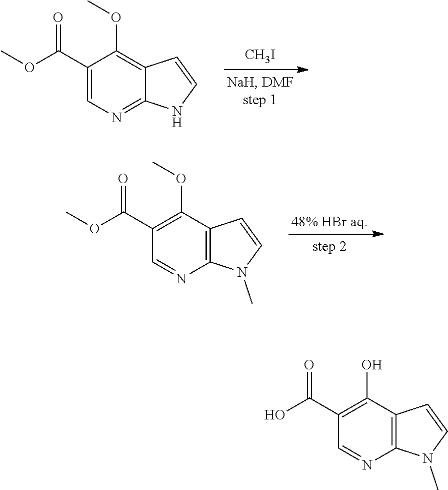 [SMILES: COC(=O)c1cnc2[nH]ccc2c1OC ; "CH3I" ; COC(=O)c1cnc2c(ccn2C)c1OC ; "48% HBr aq." ; Cn1ccc2c(O)c(C(=O)O)cnc21]

### Step 1: methyl 4-methoxy-1-methyl-1H-pyrrolo[2,3-b]pyridine-5-carboxylate

To a solution of methyl 4-methoxy-1H-pyrrolo[2,3-b]pyridine-5-carboxylate (100 mg, 0.48 mmol) in DMF (3 mL) under a nitrogen atmosphere was added NaH (60% dispersion in mineral oil, 14 mg, 0.58 mmol) at 0° C. The mixture was stirred for 1 h., then CH₃I (75 mg, 0.53 mmol) was added. The resulting mixture was stirred for 1 h., concentrated, washed with cold water (50 mL) and extracted with EtOAc (30 mL×3). The combined organic layers were dried over Na₂SO₄, filtered and concentrated. The resulting residue was purified by Prep-HPLC on a C18 column eluting with MeOH/H₂O (5-95%, with 0.1% FA) to yield the title compound (55 mg, 51% yield) as a white solid. ¹H NMR (400 MHZ, DMSO-d₆) δ ppm 8.51 (s, 1H), 7.49 (d, J=3.6 Hz, 1H), 6.91 (d, J=3.6 Hz, 1H), 4.28 (s, 3H), 3.80 (d, J=2.8 Hz, 6H). LCMS calc. for C₁₁H₁₃N₂O₃ [M+H]⁺: m/z=221.3; Found: 221.3.

### Step 2: 4-hydroxy-1-methyl-1H-pyrrolo[2,3-b]pyridine-5-carboxylic acid

A solution of methyl 4-methoxy-1-methyl-1H-pyrrolo[2,3-b]pyridine-5-carboxylate (20 mg, 0.09 mmol) in 48% HBr aq. (1.0 mL) was stirred at 140° C. for 2 h. The reaction mixture was concentrated and freeze-dried to yield the title compound (25 mg, crude) as a yellow solid. ¹H NMR (400 MHZ, DMSO-d₆) δ ppm 11.05 (d, J=13.6 Hz, 1H), 9.07 (d, J=2.2 Hz, 1H), 8.84 (d, J=13.6 Hz, 1H), 7.38 (d, J=2.2 Hz, 1H), 4.21 (d, J=7.1 Hz, 2H), 4.13 (d, J=7.1 Hz, 2H), 1.27 (d, J=7.1 Hz, 3H), 1.24 (d, J=6.0 Hz, 3H). LCMS calc. for C₉H₉N₂O₃ [M+H]⁺: m/z=193.1; Found: 193.2.

### INT C28: 7-Hydroxythiazolo[4,5-b]pyridine-6-carboxylic acid

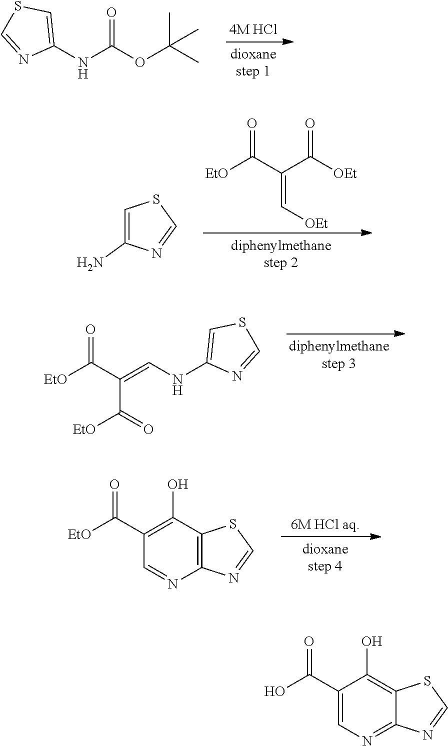

### Step 1: thiazol-4-amine hydrochloride

To a dried flask were added tert-butyl thiazol-4-ylcarbamate (5.0 g, 24.97 mmol) and 4 M HCl/dioxane (30 mL). The mixture was stirred at r.t for 16 h. Solvent was removed under reduced pressure to yield the title compound (6.4 g) as a HCl salt. LCMS calc. for C₃H₅N₂S [M+H]⁺: m/z=101.2; Found: 101.2.

### Step 2: diethyl 2-((thiazol-4-ylamino)methylene)malonate

A solution of thiazol-4-amine (3.2 g, 31.96 mmol) in diethyl 2-(ethoxymethylene)malonate (20 mL) was heated at reflux for 2 h. and concentrated. The residue was purified by Prep-HPLC on a C18 column eluting with MeOH/H₂O (30%-50%, with 0.1% FA) to yield the title compound (1.3 g, 15% yield) as a white solid. ¹H NMR (400 MHZ, DMSO-d₆) δ ppm 11.05 (d, J=13.6 Hz, 1H), 9.07 (d, J=2.2 Hz, 1H), 8.84 (d, J=13.6 Hz, 1H), 7.38 (d, J=2.2 Hz, 1H), 4.21 (d, J=7.1 Hz, 2H), 4.13 (d, J=7.1 Hz, 2H), 1.27 (d, J=7.1 Hz, 3H), 1.24 (d, J=6.0 Hz, 3H). LCMS calc. for C₁₁H₁₅N₂O₄S [M+H]⁺: m/z=271.1; Found: 271.2.

### Step 3: ethyl 7-hydroxythiazolo[4,5-b]pyridine-6-carboxylate

A mixture of diethyl 2-((thiazol-4-ylamino)methylene)malonate (1.3 g, 4.81 mmol) and diphenylmethane (10 ml) was heated at 240° C. for 8 h. The reaction mixture was cooled to r.t. and concentrated. The residue was purified by Prep-HPLC on a C18 column eluting with MeOH/H₂O (5%-20%, with 0.1% NH₄HCO₃) to yield the title compound (66 mg, 6% yield) as a yellow solid. ¹H NMR (400 MHZ, DMSO-d₆) δ ppm 9.20 (s, 1H), 8.55 (s, 1H), 4.16 (q, J=7.0 Hz, 2H), 1.26 (t, J=7.0 Hz, 3H). LCMS calc. for C₉H₉N₂O₃S [M+H]⁺: m/z=225.1; Found: 225.1.

### Step 4: 7-hydroxythiazolo[4,5-b]pyridine-6-carboxylic acid

A solution of ethyl 7-hydroxythiazolo[4,5-b]pyridine-6-carboxylate (56 mg, 0.249 mmol) in HCl aq. (2.0 mL, 6 N) and dioxane (2 mL) was stirred at 95° C. for 3 h. The mixture was concentrated and freeze-dried to yield the title compound (50 mg, crude) as a white solid. ¹H NMR (400 MHZ, DMSO-d₆) δ ppm 9.75 (s, 1H), 8.74 (s, 1H). LCMS calc. for C₇H₅N₂O₃S [M+H]⁺: m/z=197.1; Found: 197.1.

### INT C29: 4-Hydroxypyridazine-3-carboxylic acid

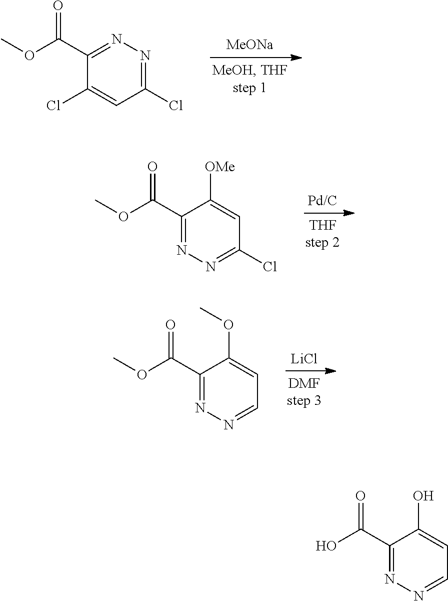

### Step 1: methyl 6-chloro-4-methoxypyridazine-3-carboxylate

To a solution of methyl 4,6-dichloropyridazine-3-carboxylate (2.0 g, 9.67 mmol) in THF (15 mL) was added MeONa in MeOH (25% wt, 10 mmol, 2.2 mL). The mixture was stirred at r.t. overnight, diluted with H₂O (80 mL) and extracted with EtOAc (80 mL×3). The combined organic lawyers were washed with brine, dried over Na₂SO₄, filtered and concentrated. The residue was purified by flash chromatography on a silica gel column eluting with EtOAc/PE (10%) to afford the title compound (1.1 g, 56% yield) as a brown solid. LCMS calc. for C₇H₈ClN₂O₃ [M+H]⁺: m/z=203.0; Found: 203.3.

### Step 2: methyl 4-methoxypyridazine-3-carboxylate

To a solution of methyl 6-chloro-4-methoxypyridazine-3-carboxylate (220 mg, 1.09 mmol) in THF (10 mL) was added Pd/C (10% wt, 50 mg). The mixture was stirred at r.t under H₂ atmosphere overnight. The mixture was filtered and concentrated under reduced pressure. The resulting residue was purified by Prep-HPLC on a C18 column eluting with MeCN/H₂O (60%-70%, with 0.1% FA) to afford the title compound (40 mg, 22% yield) as a white solid. LCMS calc. for C₇H₉N₂O₃ [M+H]⁺: m/z=169.0; Found: 169.2.

### Step 3: 4-hydroxypyridazine-3-carboxylic acid

To a solution of methyl 4-methoxypyridazine-3-carboxylate (200 mg, 1.19 mmol) in DMF (5 mL) was added LiCl (100 mg, 2.38 mmol). The mixture was stirred at r.t for 4 h., filtered and concentrated under reduced pressure. The residue was diluted with H₂O (1 mL), adjusted to pH to 1˜2 with HCl aq. (1 N), and then purified by Prep-HPLC on a C18 column eluting with MeCN/H₂O (10%, with 0.1% FA) to afford the title compound (80 mg, 48% yield) as a white solid. LCMS calc. for C₅H₅N₂O₃ [M+H]⁺: m/z=141.0; Found: 141.2.

### INT C30: 6-Chloro-4-hydroxypyridazine-3-carboxylic acid

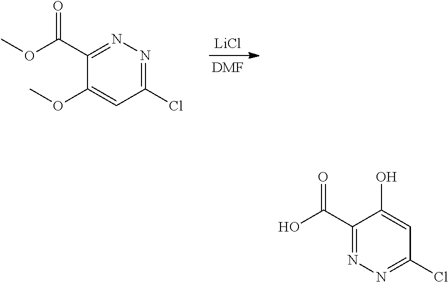

To a solution of methyl 6-chloro-4-methoxypyridazine-3-carboxylate (100 mg, 0.5 mmol) in DMF (5 mL) was added LiCl (46 mg, 1.09 mmol). The mixture was stirred at r.t for 4 h., filtered and concentrated under reduced pressure. The residue was diluted with H₂O (1 mL), adjusted to pH to 1˜2 with HCl aq. (1 N), and then purified by Prep-HPLC on a C18 column eluting with MeCN/H₂O (10%, with 0.1% FA) to afford the title compound (30 mg, 35% yield) as a light yellow solid. LCMS calc. for C₅H₄ClN₂O₃ [M+H]⁺: m/z=175.0; Found: 175.1.

### INT C31: 4-Hydroxypyrrolo[1,2-b]pyridazine-3-carboxylic acid

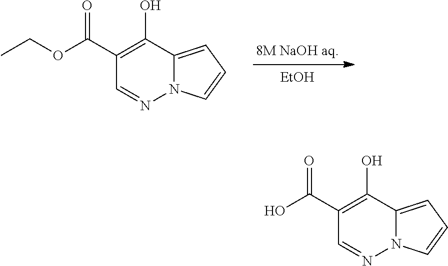

A mixture of ethyl 4-hydroxypyrrolo[1,2-b]pyridazine-3-carboxylate (824 mg, 4.0 mmol) in NaOH aq. (8 N, 3 mL) and EtOH (10 mL) was stirred at 80° C. for 6 h. The resulting mixture was concentrated and diluted with water (10 mL). The mixture was adjusted to pH to 6 with HCl aq. (1 N). The solid precipitated was collected by filtration to afford the title compound (700 mg, 92% yield) as a gray solid. ¹H NMR (400 MHZ, DMSO-d₆) δ ppm 8.27 (s, 1H), 7.91 (dd, J=2.6, 1.5 Hz, 1H), 6.91 (dd, J=4.4, 1.6 Hz, 1H), 6.81 (dd, J=4.4, 2.6 Hz, 1H). LCMS calc. for C₈H₇N₂O₃ [M+H]⁺: m/z=179.0; Found: 179.0.

### INT C32: 7-Hydroxyimidazo[1,2-b]pyridazine-6-carboxylic acid

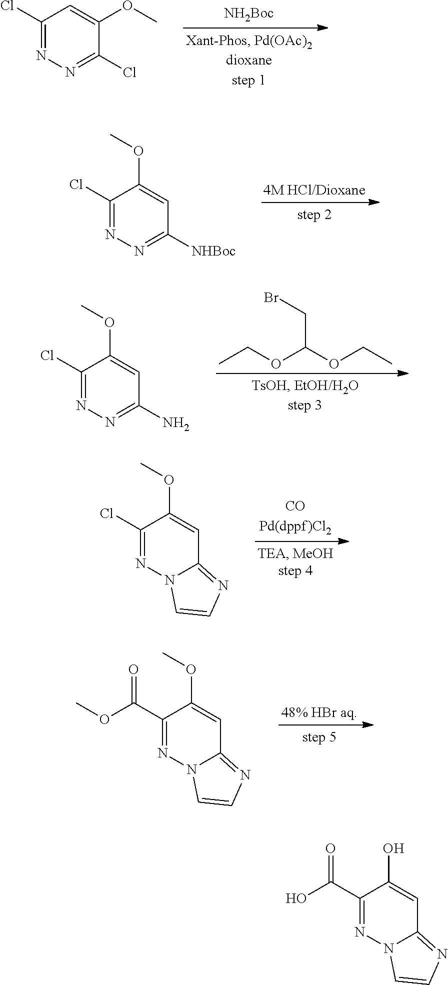

### Step 1: tert-butyl (6-chloro-5-methoxypyridazin-3-yl)carbamate

A mixture of 3,6-dichloro-4-methoxypyridazine (1 g, 5.59 mmol), NH₂Boc (851 mg, 7.26 mmol), Xant-Phos (484 mg, 0.84 mmol) and Pd(OAc)₂ (87.8 mg, 0.39 mmol) in dioxane (10 mL) was heated in a microwave oven at 100° C. for 2 h. The resulting mixture was cooled to r.t. and extracted with EtOAc (100 mL×3). The combined organic layers were washed with brine, dried over Na₂SO₄, filtered and concentrated under reduced pressure. The residue was purified by flash chromatography on a silica gel column eluting with EtOAc/PE (20%) to afford the title compound (420 mg, 29% yield) as a white solid. LCMS calc. for C₁₀H₁₅ClN₃O₃ [M+H]⁺: m/z=260.1; Found: [M+H−56]⁺: 204.1.

### Step 2: 6-chloro-5-methoxypyridazin-3-amine

A mixture of tert-butyl (6-chloro-5-methoxypyridazin-3-yl)carbamate (1 g) in 4 M HCl/1,4-dioxane (10 mL) was heated at 40° C. for 12 h. and concentrated to afford the title compound (1.2 g crude) as a white solid. LCMS calc. for C₅H₇ClN₃O [M+H]⁺: m/z=160.0; Found: 160.2.

### Step 3: 6-chloro-7-methoxyimidazo[1,2-b]pyridazine

To a solution of 6-chloro-5-methoxypyridazin-3-amine (800 mg, crude, 2.51 mmol) and 2-bromo-1,1-diethoxyethane (2.95 g, 7.52 mmol) in EtOH/H₂O (5:1, 6 mL) was added TsOH (8.6 mg, 0.08 mmol). The mixture was stirred at 80° C. for 12 h. and quenched with H₂O (5 mL). The mixture was extracted with DCM (5 mL×3), and the organic layers were combined and concentrated. The residue was purified by Prep-HPLC on a C18 column eluting with MeCN/H₂O (5%-95%, with 0.1% FA) to afford the title compound (84 mg, 18% yield) as a white solid. ¹H NMR (400 MHZ, CDCl₃) δ ppm 7.79 (s, 1H), 7.66 (s, 1H), 7.45 (s, 1H), 4.03 (s, 3H). LCMS calc. for C₇H₇ClN₃O [M+H]⁺: m/z=184.0; Found: 184.3.

### Step 4: methyl 7-methoxyimidazo[1,2-b]pyridazine-6-carboxylate

To a 30-mL pressure reactor was added 6-chloro-7-methoxyimidazo[1,2-b]pyridazine (84 mg, 0.55 mmol), MeOH (10 mL), TEA (112 mg, 1.11 mmol), and Pd(dppf)Cl₂ (127 mg, 0.18 mmol). The resulting mixture was stirred at 80° C. for 12 h. under CO atmosphere (30 atm) and then filtered. The filtrate was extracted with EtOAc (100 mL×2). The organic layers were combined, washed with brine, dried over anhydrous Na₂SO₄, filtered and concentrated. The residue was purified by Prep-HPLC on a C18 column eluting with MeCN/H₂O (5%-95%, with 0.1% FA) to afford the title compound (20 mg, 21% yield) as a white solid. ¹H NMR (400 MHz, DMSO-d₆) δ ppm 8.19 (s, 1H), 7.75 (s, 1H), 7.71 (d, J=1.3 Hz, 1H), 3.93 (s, 6H). LCMS calc. for C₉H₁₀N₃O₃ [M+H]⁺: m/z=208.1; Found: 208.2.

### Step 5: 7-hydroxyimidazo[1,2-b]pyridazine-6-carboxylic acid

A mixture of methyl 7-methoxyimidazo[1,2-b]pyridazine-6-carboxylate (20 mg, 96.5 μmol) in 48% HBr aq. (3 mL) was stirred at 140° C. for 2 h. and concentrated. The residue was freeze-dried to afford the title compound (30 mg crude) as a white solid. ¹H NMR (400 MHZ, DMSO-d₆) δ ppm 8.39 (d, J=2.2 Hz, 1H), 8.04 (d, J=2.3 Hz, 1H), 7.29 (s, 1H). LCMS calc. for C₇H₆N₃O₃ [M+H]⁺: m/z=180.0; Found: 180.1.

### INT C33: 3-Hydroxythieno[2,3-b]pyridine-2-carboxylic acid

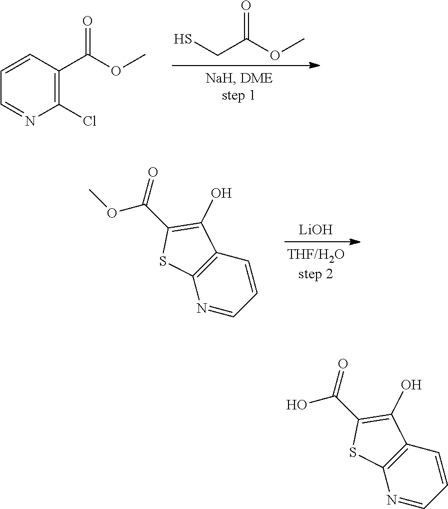

### Step 1: methyl 3-hydroxythieno[2,3-b]pyridine-2-carboxylate

To a suspension of NaH (60% dispersion in mineral oil, 608 mg, 15.2 mmol) in DME (15 mL) at 0° C. under N₂ was added methyl 2-mercaptoacetate (1.4 g, 14.6 mmol). The mixture was stirred at 0° C. for 0.5 h., then methyl 2-chloronicotinate (1 g, 5.84 mmol) in DME (5 mL) was added. The resulting mixture was stirred at 70° C. for 16 h. and concentrated. The residue was purified by Prep-HPLC on a C18 column eluting with MeCN/H₂O (60-75%, with 0.05% NH₄HCO₃) to afford the title compound (1.0 g, 82% yield) as a green solid. ¹H NMR (400 MHZ, DMSO-d₆) δ ppm 8.54 (dd, J=4.5, 1.5 Hz, 1H), 8.05 (d, J=7.8 Hz, 1H), 7.26 (dd, J=7.8, 4.6 Hz, 1H), 3.67 (s, 3H). LCMS calc. for C₉H₈NO₃S [M+H]⁺: m/z=210.0; Found: 210.2.

### Step 2: 3-hydroxythieno[2,3-b]pyridine-2-carboxylic acid

A solution of methyl 3-hydroxythieno[2,3-b]pyridine-2-carboxylate (400 mg, 1.91 mmol) and LiOH (276 mg, 11.48 mmol) in THF (8 mL) and H₂O (2 mL) was stirred at 60° C. for 16 h. The organic solvent was removed under reduced pressure and the remaining solution was acidified with HCl aq. (1 N) until pH=1. The precipitates were collected, washed with water (1.5 mL), diethyl ether (1 mL) and hexane (5 mL) to afford the title compound (150 mg, 40% yield) as a yellow solid. ¹H NMR (400 MHZ, DMSO-d₆) δ ppm 10.32 (s, 1H), 8.54 (dd, J=4.6, 1.6 Hz, 1H), 8.11 (dd, J=8.0, 1.6 Hz, 1H), 7.40 (dd, J=8.0, 4.6 Hz, 1H), 6.57 (s, 1H). LCMS calc. for C₈H₆NO₃S [M+H]⁺: m/z=196.0; Found: 196.1.

### INT C34: 4-Hydroxyfuro[2,3-b]pyridine-2-carboxylic acid

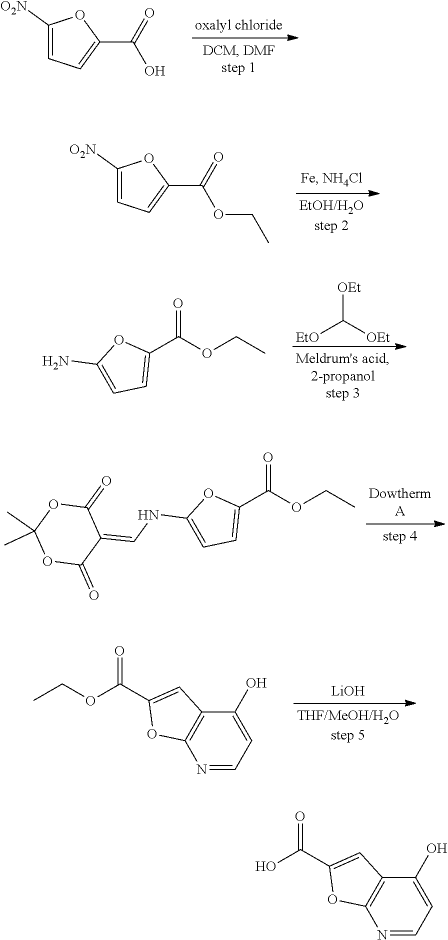

### Step 1: ethyl 5-nitrofuran-2-carboxylate

To a stirred solution of 5-nitro-furan-2-carboxylic acid (4 g, 25.48 mmol) in DCM (60 mL) was added oxalyl chloride (3.3 mL, 38.22 mmol) and DMF (1 mL). After stirring for 45 min., the reaction mixture was concentrated, and the residue was diluted with CH₂Cl₂ (50 mL) and stirred overnight. EtOH (4 mL) and triethylamine (4.4 mL) were added. After stirring at 0° C. for 15 min., the reaction mixture was warmed to r.t. and stirred for 1 h. The resulting reaction mixture was washed with saturated NaHCO₃ (20 mL), water (20 mL), brine (20 mL), dried over Na₂SO₄ and filtered. The filtrate was concentrated to afford the title compound (4.3 g, 91% yield) as a white solid. ¹H NMR (400 MHZ, DMSO-d₆) δ ppm 7.78 (d, J=3.9 Hz, 1H), 7.58 (d, J=3.9 Hz, 1H), 4.38 (q, J=7.1 Hz, 2H), 1.33 (t, J=7.1 Hz, 3H).

### Step 2: ethyl 5-aminofuran-2-carboxylate

A mixture of ethyl 5-nitrofuran-2-carboxylate (4.3 g, 23.24 mmol), Fe (6.5 g, 116.20 mmol) and NH₄Cl (123 mg, 2.32 mmol) in EtOH/H₂O (40 mL/20 mL) was refluxed for 2 h., cooled to r.t. and filtered under reduced pressure. The filtrate was concentrated. The residue was purified by Prep-HPL on a C18 column eluting with MeCN/H₂O (5%-95%, with 5% NH₄HCO₃) to afford the title compound (2.0 g, 56% yield) as a red solid. LCMS calc. for C₇H₁₀NO₃ [M+H]⁺: m/z=156.0; Found: 156.0.

### Step 3: ethyl 5-(((2,2-dimethyl-4,6-dioxo-1,3-dioxan-5-ylidene)methyl)amino)furan-2-carboxylate

To a mixture of ethyl 5-aminofuran-2-carboxylate (1 g, 6.45 mmol) in triethyl orthoformate (5 ml, 30.1 mmol) and 2-propanol (5 ml) was added Meldrum's acid (1.1 g, 7.74 mmol). The resulting mixture was stirred at 100° C. for 1.5 h. and cooled to r.t. The precipitate was collected by filtration, washed sequentially with 2-propanol (3 mL×2) and pentane (4 mL) to afford the title compound (1.2 g, 60% yield) as a brown solid. ¹H NMR (400 MHZ, CDCl₃) δ ppm 11.50 (d, J=13.4 Hz, 1H), 8.63 (d, J=13.5 Hz, 1H), 7.20 (d, J=3.7 Hz, 1H), 6.08 (d, J=3.7 Hz, 1H), 4.38 (q, J=7.1 Hz, 2H), 1.76 (s, 6H), 1.39 (t, J=7.1 Hz, 3H).

### Step 4: ethyl 4-hydroxyfuro[2,3-b]pyridine-2-carboxylate

A solution of ethyl 5-(((2,2-dimethyl-4,6-dioxo-1,3-dioxan-5-ylidene)methyl)amino)furan-2-carboxylate (1.2 g, 3.88 mmol) in Dowtherm A (5 mL) was stirred at 200° C. for 1 h. and then at 220° C. for 15 min. After the mixture was cooled to r. t., Et₂₀ (2 ml) was added and the precipitate was collected by filtration, washed with Et₂₀ (2 mL×2) to afford the title compound as a brown solid (560 mg, 70% yield). ¹H NMR (400 MHz, DMSO-d₆) δ ppm 8.20 (d, J=5.6 Hz, 1H), 7.72 (s, 1H), 6.79 (d, J=5.6 Hz, 1H), 4.36 (q, J=7.1 Hz, 2H), 1.34 (t, J=7.1 Hz, 3H).

### Step 5: 4-hydroxyfuro[2,3-b]pyridine-2-carboxylic acid

To a mixture of ethyl 4-hydroxyfuro[2,3-b]pyridine-2-carboxylate (100 mg, 0.48 mmol) in MeOH (0.5 ml) and THF (0.5 ml) was added a solution of LiOH·H₂O (40 mg, 0.96 mmol) in water (0.5 ml) and the resulting mixture was stirred at 60° C. overnight. The mixture was cooled to r.t., and concentrated. The residue was acidified to pH=2 with HCl aq. (2 N). The precipitate was collected by filtration, washed with water (10 mL), pentane (10 mL), and the filtrate was dried over Na₂SO₄, filtered and concentrated under reduced pressure to afford the title compound (45 mg, 52% yield) as a yellow solid. LCMS calc. for CH₆NO₄ [M+H]⁺: m/z=180.0; Found: 180.0.

### INT C35: 3-Hydroxythieno[2,3-c]pyridine-2-carboxylic acid

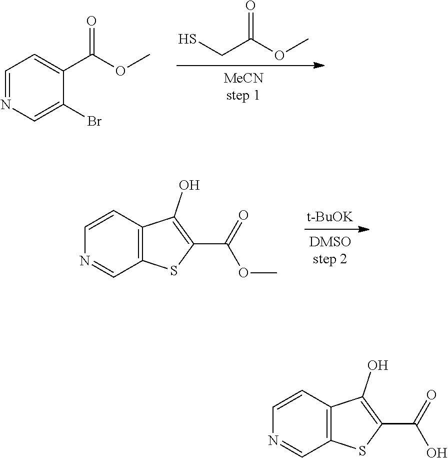

### Step 1: methyl 3-hydroxythieno[2,3-c]pyridine-2-carboxylate

A mixture of methyl 3-bromoisonicotinate (500 mg, 2.31 mmol), K₂CO₃ (478 mg, 3.47 mmol) and methyl thioglycolate (0.2 mL, 244 mg, 2.31 mmol) in MeCN (12 mL) was refluxed for 18 h. After solvent was evaporated, the residue was dissolved in water (5 mL) and adjusted to pH to 4 with acetic acid. The precipitates were collected by filtration and dried under vacuum to afford the title compound (280 mg, 62% yield) as a white solid. LCMS calc. for C₉H₈NO₃S [M+H]⁺: m/z=210.0; Found: 209.8.

### Step 2: 3-hydroxythieno[2,3-c]pyridine-2-carboxylic acid

A solution of potassium tert-butoxide (1.07 g, 9.60 mmol) in DMSO (4.8 mL) was stirred at r.t. for 10 min., then methyl 3-hydroxythieno[2,3-c]pyridine-2-carboxylate (200 mg, 0.96 mmol) was added. The mixture was stirred at r.t. for 1 h. Ice-water (5 mL) was added and the mixture was acidified with HCl aq. (1 N) to PH˜ 1. The precipitates were collected by filtration, and washed with water (5 mL), cold diethyl ether (2 mL) and hexane (2 mL) to afford the title compound (65 mg, 35% yield) as a yellow solid. LCMS calc. for C₈H₆NO₃S [M+H]⁺: m/z=196.0; Found: 195.9.

### INT C36: 3-Hydroxyimidazo[1,2-a]pyrazine-2-carboxylic acid

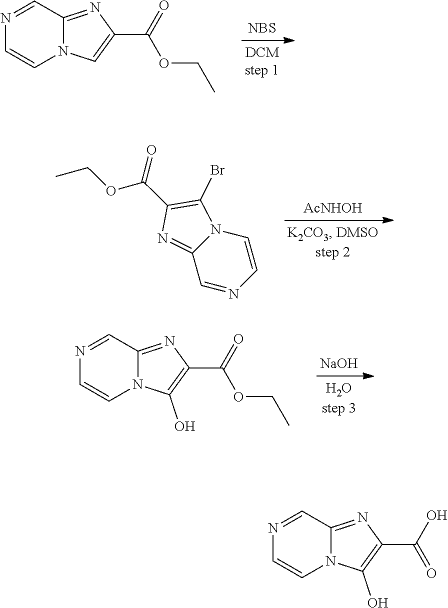

### Step 1: ethyl 3-bromoimidazo[1,2-a]pyrazine-2-carboxylate

To a solution of ethyl imidazo[1,2-a]pyrazine-2-carboxylate (1 g, 5.23 mmol) in DCM (10 mL) was added NBS (931.9 mg, 5.23 mmol) at r.t. After the mixture was stirred at r.t for 16 h., H₂O (5 mL) was added. The mixture was extracted with DCM (5 mL×3). The combined organic layers were washed with saturated NH₄Cl aq. (10 mL), brine (10 mL), dried over Na₂SO₄, filtered and concentrated. The residue was purified by flash chromatography on a silica gel column eluting with EtOAc/PE (20%) to afford the title compound (1.2 g, 85% yield) as a yellow solid. ¹H NMR (400 MHz, DMSO-d₆) δ ppm 9.20 (d, J=1.4 Hz, 1H), 8.48 (dd, J=4.7, 1.5 Hz, 1H), 8.11 (d, J=4.7 Hz, 1H), 4.39 (t, J=7.1 Hz, 2H), 1.36 (dd, J=9.2, 5.0 Hz, 3H). LCMS calc. for CH₉BrN₃O₂ [M+H]⁺: m/z=270.0; Found: 270.0.

### Step 2: ethyl 3-hydroxyimidazo[1,2-a]pyrazine-2-carboxylate

To a mixture of ethyl 3-bromoimidazo[1,2-a]pyrazine-2-carboxylate (400 mg, 1.48 mmol), AcNHOH (334.8 mg, 4.46 mmol) in DMSO (3 mL) was added K₂CO₃ (1.02 g, 7.40 mmol). After the mixture was stirred at 80° C. for 2 h., H₂O (5 mL) was added. The mixture was extracted with DCM (5 mL×3). The combined organic layers were washed with saturated NH₄Cl aq. (10 mL), brine (10 mL), dried over Na₂SO₄, filtered and concentrated. The residue was purified by Prep-HPLC on a C18 column eluting with MeCN/H₂O (5-95%, with 0.1% NH₄HCO₃) to afford the title compound (300 mg, 97% yield) as a yellow solid. LCMS calc. for C₉H₁₀N₃O₃ [M+H]⁺: m/z=208.1; Found: 208.0.

### Step 3: 3-hydroxyimidazo[1,2-a]pyrazine-2-carboxylic acid

To a solution of ethyl 3-hydroxyimidazo[1,2-a]pyrazine-2-carboxylate (200 mg, 0.96 mmol) in H₂O (3 mL) was added NaOH (69.5 mg, 2.89 mmol). The mixture was stirred at 90° C. for 2 h., ice-water (40 mL) was added and the mixture was acidified with HCl aq. (1 N) to PH˜ 1. The precipitates were collected by filtration, washed with water (2 mL), cold diethyl ether (2 mL) and hexane (5 mL) to afford the title compound (20 mg, 17% yield) as a yellow solid. LCMS calc. for C₇H₄N₃O₃ [M−H]⁻: m/z=178.0; Found: 178.0.

### INT C_{37: 5}-Methyl-4-oxo-1,4-dihydropyridazine-3-carboxylic acid

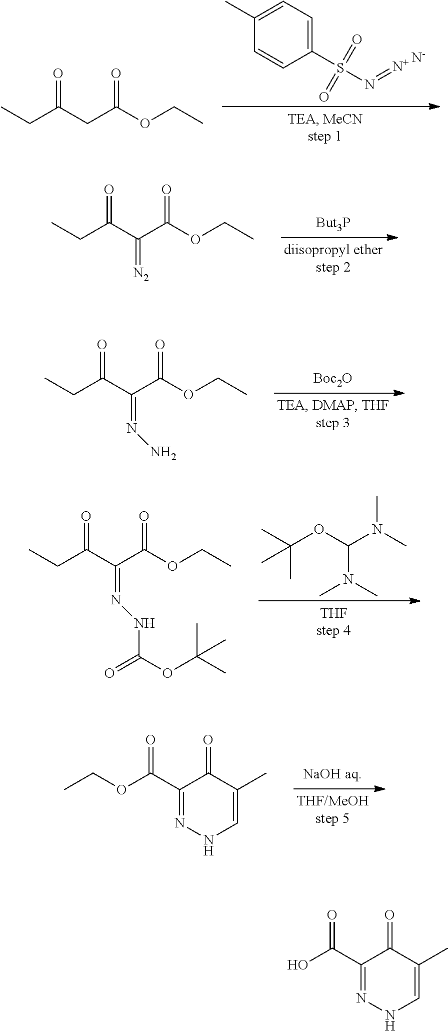

### Step 1: ethyl 2-diazo-3-oxopentanoate

To a solution of ethyl 3-oxopentanoate (10 g, 0.07 mol) in MeCN (100 mL) was added TEA (18.74 g, 0.19 mol) and 4-methylbenzenesulfonyl azide (11.63 g, 0.06 mol) at 0° C. The mixture was stirred at r.t. for 2 h., and concentrated. After H₂O (300 mL) was added, the mixture was extracted with EtOAc (200 mL×3). The combined organic layers were concentrated. The residue was purified by flash chromatography on a silica gel column eluting with EtOAc/PE (5%) to afford the title compound (8.0 g, 68% yield) as a yellow oil. LCMS calc. for C₇H₁₁N₂O₃ [M+H]⁺: m/z=171.1; Found: 171.0.

### Step 2: ethyl 2-hydrazineylidene-3-oxopentanoate

To a solution of ethyl 2-diazo-3-oxopentanoate (8.0 g, 0.05 mol) in diisopropyl ether (80 mL) was added tributylphosphine (10.85 g, 0.05 mol). The mixture was stirred at r.t. for 2 h., concentrated, diluted with H₂O (300 mL), extracted with EtOAc (200 mL×3). The combined organic layers were dried over Na₂SO₄, filtered and concentrated to give the title product (15.7 g, crude) as a yellow oil. LCMS calc. for C₇H₁₃N₂O₃ [M+H]⁺: m/z=173.1; Found: 173.0.

### Step 3: tert-butyl-2-(1-ethoxy-1,3-dioxopentan-2-ylidene) hydrazine-1-carboxylate

To a solution of ethyl 2-hydrazineylidene-3-oxopentanoate (1.0 g, 5.81 mmol) in THF (5 mL) was added di-tert-butyl dicarbonate (1.3 g, 5.98 mmol), TEA (1.7 g, 17.09 mmol), and DMAP (72 mg, 0.58 mmol). The mixture was stirred at r.t. overnight, concentrated, diluted with H₂O (100 mL) and extracted with EtOAc (100 mL×3). The combined organic layers were dried over Na₂SO₄, filtered and concentrated. The residue was purified by flash chromatography on a silica gel column eluting with EtOAc/PE (5%) to afford the title compound (520 mg, 35% yield) as a yellow solid. LCMS calc. for C₁₂H₂₁N₂O₅ [M+H]⁺: m/z=273.1. Found: [M+H−56]⁺: 217.2.

### Step 4: ethyl 5-methyl-4-oxo-1,4-dihydropyridazine-3-carboxylate

To a solution of tert-butyl 2-(1-ethoxy-1,3-dioxopentan-2-ylidene) hydrazine-1-carboxylate (300 mg, 1.10 mmol) in THF (3 mL) was added 1-tert-butoxy-N,N,N′,N′-tetramethylmethanediamine (385 mg, 2.21 mmol). The mixture was stirred at 70° C. for 1 h., concentrated, diluted with H₂O (50 mL) and extracted with EtOAc (50 mL×3). The combined organic layers were dried over Na₂SO₄, filtered and concentrated. The residue was purified by Prep-TLC eluting with EtOAc/PE (5%) to afford the title compound (140 mg, 70% yield) as a white solid. LCMS calc. for C₈H₁₁N₂O₃ [M+H]⁺: m/z=183.1; Found: 183.2.

### Step 5:5-methyl-4-oxo-1,4-dihydropyridazine-3-carboxylic acid

To a solution of ethyl 5-methyl-4-oxo-1,4-dihydropyridazine-3-carboxylate (100 mg, 0.55 mmol) in THF (2 mL) and MeOH (2 mL) was added aq. NaOH (2N, 1.5 mL). The mixture was stirred at r.t. overnight, and then acidified to pH=4 with HCl aq. (1 N). The precipitates were collected by filtration to give the title product (48 mg, 51% yield) as a white solid. LCMS calc. for C₆H₇N₂O₃ [M+H]⁺: m/z=155.0; Found: 155.2.

### INT C38: 4-Hydroxy-2-methylpyrazolo[1,5-a]pyridine-5-carboxylic acid

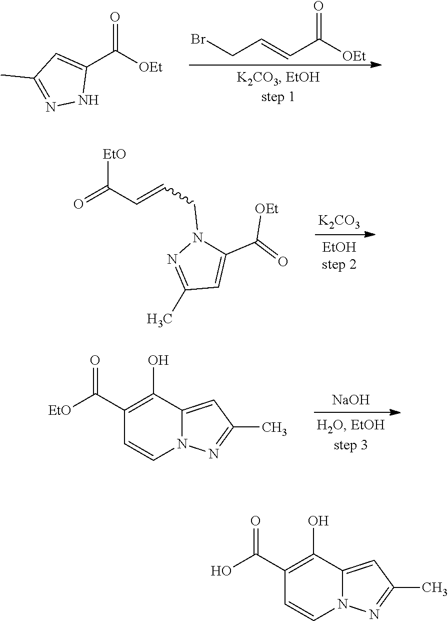

### Step 1: ethyl 1-(4-ethoxy-4-oxobut-2-en-1-yl)-3-methyl-1H-pyrazole-5-carboxylate

To a solution of ethyl 3-methyl-1H-pyrazole-5-carboxylate (10 g, 65 mmol) in ethanol (150 mL) was added ethyl 4-bromobut-2-enoate (25 g, 130 mmol) and K₂CO₃ (18 g, 130 mmol). The reaction mixture was heated at 78° C. for 12 h., diluted with H₂O (200 mL) and extracted with EtOAc (50 mL×3). The combined organic layers were dried over Na₂SO₄, filtered and concentrated under reduced pressure. The residue was purified by flash chromatography on a silica gel column eluting with EtOAc/PE (30%) to give the title compound (6.5 g, 38% yield) as a yellow solid. LCMS calc. for C₁₃H₁₉N₂O₄ [M+H]⁺: m/z=267.1; Found: 267.0.

### Step 2: ethyl 4-hydroxy-2-methylpyrazolo[1,5-a]pyridine-5-carboxylate

To a solution of ethyl 1-(4-ethoxy-4-oxobut-2-en-1-yl)-3-methyl-1H-pyrazole-5-carboxylate (2 g, 7.5 mmol) in ethanol (20 mL) was added K₂CO₃ (3.1 g, 22.6 mmol). The mixture was stirred at 90° C. for 3 days, diluted with H₂O (100 mL) and extracted with EtOAc (50 mL×3). The combined organic layers were dried over Na₂SO₄, filtered and concentrated. The residue was purified by flash chromatography on a silica gel column eluting with EtOAc/PE (20%) to give the title compound (200 mg, 12% yield) as a yellow solid. LCMS calc. for C₁₁H₁₃N₂O₃ [M+H]⁺: m/z=221.1; Found: 221.0.

### Step 3: 4-hydroxy-2-methylpyrazolo[1,5-a]pyridine-5-carboxylic acid

To a solution of ethyl 4-hydroxy-2-methylpyrazolo[1,5-a]pyridine-5-carboxylate (200 mg, 0.91 mmol) in ethanol (1 mL) and H₂O (1 mL) was added NaOH (73 mg, 1.82 mmol). The mixture was stirred at r.t. for 2 h., and concentrated. The residue was diluted with H₂O (10 mL), and adjusted to pH to 5-6 with HCl aq. (1 N). The precipitates formed were collected by filtration, dried under vacuum to afford the title compound (120 mg, 69% yield) as a white solid. LCMS calc. for C₉H₉N₂O₃ [M+H]⁺: m/z=193.1; Found: 193.2.

### INT C39: 6-Hydroxy-1-methyl-1H-indole-5-carboxylic acid

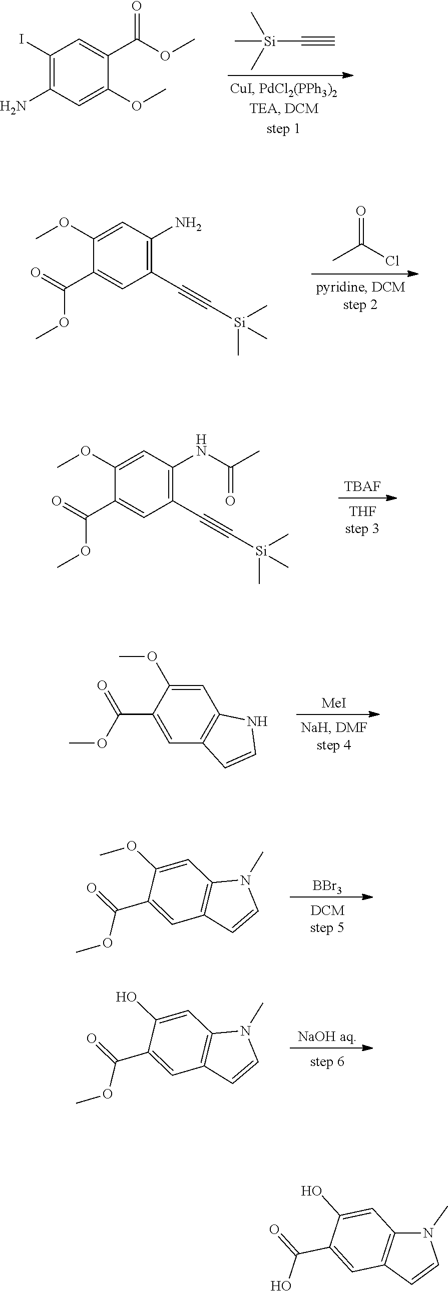

### Step 1: methyl 4-amino-2-methoxy-5-((trimethylsilyl)ethynyl)benzoate

To a solution of methyl 4-amino-5-iodo-2-methoxybenzoate (400 mg, 1.30 mmol) in DCM (5 mL) was added copper iodide (2.47 mg, 13 μmol), PdCl₂(PPh₃)₂ (4.56 mg, 6.5 μmol), triethylamine (197 mg, 1.95 mmol) and trimethylsilyl acetylene (160 mg, 1.6 mmol) sequentially. The mixture was stirred at r.t. under N₂ for 4 h., and concentrated. The residue was diluted with ethyl acetate (10 mL) and water (10 mL), and filtered through a pad of Celite. The organic layer was washed with water, brine, dried over Na₂SO₄, filtered and concentrated. The residue was purified by flash chromatography on a silica gel column eluting with EtOAc/PE (30%-100%) to afford the title compound (320 mg, 89% yield) as a yellow solid. LCMS calc. for C₁₄H₂₀NO₃Si [M+H]⁺: m/z=278.1; Found: 278.0.

### Step 2: Methyl 4-acetamido-2-methoxy-5-((trimethylsilyl)ethynyl)benzoate

To a solution of methyl 4-amino-2-methoxy-5-((trimethylsilyl)ethynyl)benzoate (320 mg, 1.0 mmol) and pyridine (0.18 mL) in DCM (5 mL) was added acetyl chloride (0.086 mL, 1.2 mmol) at 0° C. The mixture was stirred at r.t. for 1 h., quenched with water (10 mL), and extracted with DCM (30 mL×3). The combined organic layers were dried over Na₂SO₄, filtered and concentrated. The residue was purified by flash chromatography on a silica gel column eluting with EtOAc/PE (0-10%) to afford the title compound (290 mg, 76% yield) as a yellow solid. LCMS calc. for C₁₆H₂₂NO₄Si [M+H]⁺: m/z=320.1; Found: 320.0.

### Step 3: methyl 6-methoxy-1H-indole-5-carboxylate

A mixture of tetrabutylammonium fluoride (3 mL, 1.0 M in THF) and methyl 4-acetamido-2-methoxy-5-((trimethylsilyl)ethynyl)benzoate (290 mg, 0.86 mmol) was stirred at 70° C. for 4 h., cooled to r.t., and concentrated. The residue was diluted with water (10 mL). The solid was collected by filtration and washed with water (10 mL). The crude solid was purified by flash chromatography on a silica gel column eluting with EtOAc/PE (12%) to afford the title compound (97 mg, 50% yield) as a yellow solid. LCMS calc. for C₁₁H₁₂NO₃ [M+H]⁺: m/z=206.1; Found: 206.0.

### Step 4. methyl 6-methoxy-1-methyl-1H-indole-5-carboxylate

To a solution of methyl 6-methoxy-1H-indole-5-carboxylate (90 mg, 0.44 mmol) in DMF (2 mL) was added NaH (60% dispersion in mineral oil, 26.3 mg, 0.66 mmol) at 0° C. The mixture was stirred for 0.5 h., and then Mel (93.72 mg, 0.66 mmol) was added. The resulting mixture was stirred at r.t. for 2 h., quenched with H₂O (5 mL), and extracted with EtOAc (5 mL×3). The combined organic layers were washed with sat. NH₄Cl aq. (10 mL), brine (10 mL), dried over Na₂SO₄, filtered and concentrated. The crude product was purified by flash chromatography on a silica gel column eluting with EtOAc/PE (15%) to afford the title compound (80 mg, 88% yield) as a yellow solid. LCMS calc. for C₁₂H₁₄NO₃ [M+H]⁺: m/z=220.1; Found: 220.0.

### Step 5: methyl 6-hydroxy-1-methyl-1H-indole-5-carboxylate

To a solution of methyl 6-methoxy-1-methyl-1H-indole-5-carboxylate (80 mg, 0.39 mmol) in DCM (1 mL) was added BBr₃ (2M in DCM, 0.78 mL)) at 0° C. The mixture was stirred at 0° C. for 4 h., quenched with H₂O (5 mL) and extracted with DCM (5 mL×3). The combined organic layers were washed with sat. NH₄Cl aq. (10 mL), brine (10 mL), dried over Na₂SO₄, filtered and concentrated. The crude product was purified by flash chromatography on a silica gel column eluting with EtOAc/PE (50%) to afford the title compound (35 mg, 50% yield) as a yellow solid. LCMS calc. for C₁₁H₁₂NO₃ [M+H]⁺: m/z=206.1; Found: 206.0.

### Step 6: 6-hydroxy-1-methyl-1H-indole-5-carboxylic acid

A solution of methyl 6-hydroxy-1-methyl-1H-indole-5-carboxylate (35 mg, 0.17 mmol) in NaOH aq. (2 M, 2 mL) was heated at 70° C. for 4 h. Upon completion (TLC analysis), ice water (10 mL) was added and the mixture was acidified with HCl aq. (1 N) until pH=1. The precipitate formed was collected, washed with water (2 mL), cold diethyl ether (2 mL) and hexane (5 mL), and dried to afford the title compound (12 mg, 43% yield) as a yellow solid. LCMS calc. for C₁₀H₁₀NO₃ [M+H]⁺: m/z=192.1; Found: 192.0.

### INT C40: 7-hydroxythiazolo[4,5-c]pyridine-6-carboxylic acid

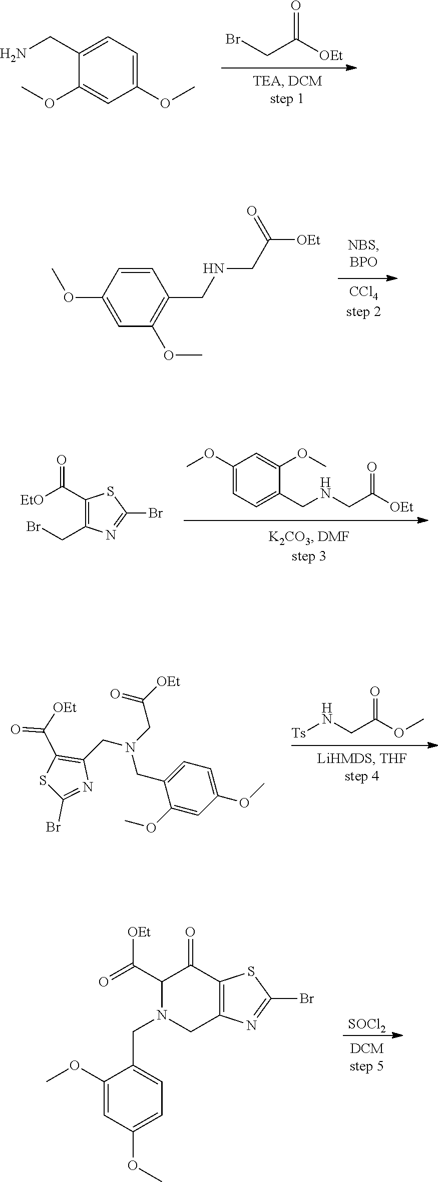

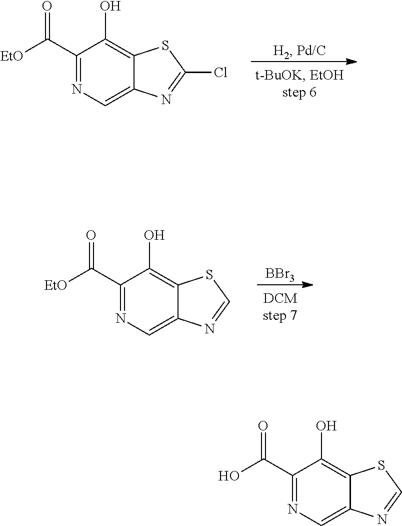 [SMILES: CCOC(=O)c1ncc2nc(Cl)sc2c1O ; "H2, Pd/C" ; CCOC(=O)c1ncc2ncsc2c1O ; "BBr3" ; O=C(O)c1ncc2ncsc2c1O]

### Step 1: ethyl (2,4-dimethoxybenzyl)glycinate

To a solution of (2,4-dimethoxyphenyl) methanamine (20 g, 119.6 mmol) in DCM (200 mL) was added ethyl 2-bromoacetate (20 g, 119.6 mmol) and TEA (12.1 g, 119.6 mmol). The mixture was stirred at r.t. overnight, quenched with water (50 mL) and extracted with DCM (200 mL×3). The combined organic layers were dried over Na₂SO₄, filtered and concentrated under reduced pressure. The residue was purified by flash chromatography on a silica gel column eluting with EtOAc/PE (3%) to afford the title compound (19 g, 63% yield) as a yellow oil. LCMS calc. for C₁₃H₂₀NO₄ [M+H]⁺: m/z=254.1; Found: 254.2.

### Step 2: ethyl 2-bromo-4-(bromomethyl)thiazole-5-carboxylate

To a solution of ethyl 2-bromo-4-methylthiazole-5-carboxylate (6.0 g, 24.0 mmol) in CCl₄ (80 mL) was added NBS (4.6 g, 26.4 mmol) and BPO (1.2 mg, 4.8 mmol). The mixture was stirred at 80° C. for 5 h., quenched with water (50 mL) and extracted with DCM (200 mL×3). The combined organic layers were dried over Na₂SO₄, filtered and concentrated under reduced pressure to afford the crude title compound (6.2 g) as a yellow oil which was used directly in next step without further purification. LCMS calc. for C₇H₈Br₂NO₂S [M+H]⁺: m/z=327.9; Found: 327.6.

### Step 3: ethyl 2-bromo-4-(((2,4-dimethoxybenzyl)(2-ethoxy-2-oxoethyl)amino)methyl)thiazole-5-carboxylate

To a solution of ethyl 2-bromo-4-(bromomethyl)thiazole-5-carboxylate (6.5 g, 19.7 mmol) in DMF (80 mL) was added ethyl (2,4-dimethoxybenzyl)glycinate (5.0 g, 19.7 mmol) and K₂CO₃ (5.5 g, 39.5 mmol). The mixture was stirred at r.t overnight, quenched with water (50 mL) and extracted with DCM (200 mL×3). The combined organic layers were dried over Na₂SO₄, filtered and concentrated under reduced pressure. The residue was purified by flash chromatography on a silica gel column eluting with EtOAc/PE (3%) to afford the title compound (4.1 g, 41% yield) as a yellow oil. LCMS calc. for C₂₀H₂₆BrN₂O₆S [M+H]⁺: m/z=501.1; Found: 500.6.

### Step 4: ethyl 2-bromo-5-(2,4-dimethoxybenzyl)-7-oxo-4,5,6,7-tetrahydrothiazolo[4,5-c]pyridine-6-carboxylate

To a solution of ethyl 2-bromo-4-(((2,4-dimethoxybenzyl)(2-ethoxy-2-oxoethyl) amino)methyl)thiazole-5-carboxylate (1 g, 2 mmol) in THF (10 mL) was added LiHMDS (1 M in THF, 4 mL, 4 mmol). The mixture was stirred at r.t for 1 h., quenched with H₂O (10 mL) and extracted with EtOAc (20 mL×3). The combined organic layers were concentrated. The residue was purified by Prep-HPLC on a C18 column eluting with MeCN/H₂O (45-70%, with 0.05% FA) to afford the title compound (700 mg, 77% yield) as a white solid. LCMS calc. for C₁₈H₂₀BrN₂O₅S [M+H]⁺: m/z=455.0; Found: 455.1.

### Step 5: ethyl 2-chloro-7-hydroxythiazolo[4,5-c]pyridine-6-carboxylate

To a solution of ethyl 2-bromo-5-(2,4-dimethoxybenzyl)-7-oxo-4,5,6,7-tetrahydrothiazolo[4,5-c]pyridine-6-carboxylate (700 mg, 1.2 mmol) in DCM (20 mL) was added SOCl₂ (10 mL). The mixture was stirred at r.t for 1 h., and concentrated. The residue was purified by Prep-HPLC on a C18 column eluting with MeCN/H₂O (45%-70%, with 0.05% FA) to afford the title compound (500 mg, 45% yield) as a white solid. ¹H NMR (400 MHZ, DMSO-d₆) δ ppm 11.29 (s, 1H), 8.85 (s, 1H), 4.42 (q, J=7.1 Hz, 2H), 1.37 (t, J=7.1 Hz, 3H). LCMS calc. for C₉H₈ClN₂O₃S [M+H]⁺: m/z=259.0; Found: 259.0.

### Step 6: ethyl 7-hydroxythiazolo[4,5-c]pyridine-6-carboxylate

To a solution of ethyl 2-chloro-7-hydroxythiazolo[4,5-c]pyridine-6-carboxylate (100 mg, 0.42 mmol) in EtOH (5 mL) was added Pd/C (10%, 100 mg) and t-BuOK (85 mg, 0.82 mmol). The mixture was stirred at r.t. under H₂ for 2 days. After completion, the insoluble substance was removed by filtration, and the filtrate was concentrated under reduced pressure. The residue was purified by Prep-HPLC on a C18 column eluting with MeCN/H₂O (45-70%, with 0.05% FA) to afford the title compound (20 mg, 22% yield) as a white solid. LCMS calc. for CH₉N₂O₃S [M+H]⁺: m/z=225.0; Found: 225.0.

### Step 7:7-hydroxythiazolo[4,5-c]pyridine-6-carboxylic acid

A solution of ethyl 7-hydroxythiazolo[4,5-c]pyridine-6-carboxylate (19 mg, 0.10 mmol) in DCM (2 mL) was added BBr₃ (1 M, 0.57 mL). The mixture was stirred at r.t. overnight and poured into water (3 mL). A yellow solid formed was collected by filtration to afford the title compound (15 mg, 93% yield). LCMS calc. for C₇H₅N₂O₃S [M+H]⁺: m/z=197.0; Found: 197.1.

### INT C41: 2-Chloro-7-hydroxythiazolo[4,5-c]pyridine-6-carboxylic acid

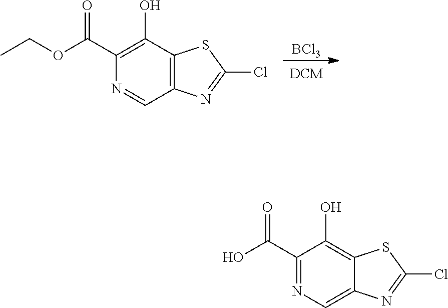 [SMILES: CCOC(=O)c1ncc2nc(Cl)sc2c1O ; "BCl3" ; O=C(O)c1ncc2nc(Cl)sc2c1O]

To a solution of ethyl 2-chloro-7-hydroxythiazolo[4,5-c]pyridine-6-carboxylate (50 mg, 0.19 mmol, INT C40 Step 5) in DCM (2 mL) was added BCl₃ (1 M, 0.57 mL). The mixture was stirred at r.t. overnight and then poured into water (3 mL). A yellow solid formed was collected by filtration to afford the title compound (15 mg, 30% yield). LCMS calc. for C₇H₄ClN₂O₃S [M+H]⁺: m/z=231.0; Found: 230.9.

### INT C42: 7-Hydroxy-1-methyl-1H-pyrrolo[3,2-c]pyridine-6-carboxylic acid

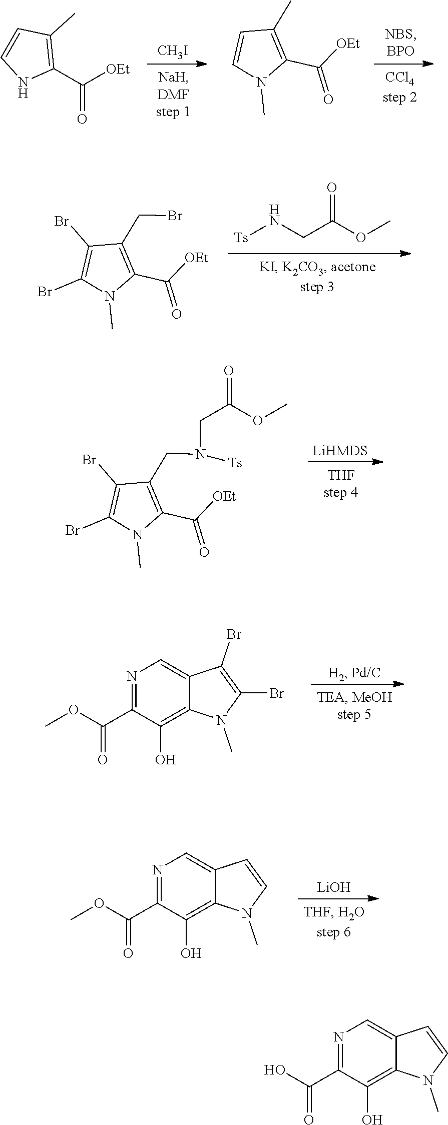

### Step 1: ethyl 1,3-dimethyl-1H-pyrrole-2-carboxylate

To a solution of ethyl 3-methyl-1H-pyrrole-2-carboxylate (5 g, 32.7 mmol) in DMF (80 mL) was added NaH (60% dispersion in mineral oil, 2 g, 49 mmol) at 0° C. The mixture was stirred at 0° C. for 30 min. and then Mel (6.9 g, 49 mmol) was added. The resulting mixture was stirred at r.t. overnight, quenched with H₂O (80 mL) and extracted with EtOAc (100 mL). The organic layer was separated, washed with brine (50 mL), dried over anhydrous Na₂SO₄, filtered and concentrated. The residue was purified by flash chromatography on a silica gel column eluting with EtOAc/PE (10%) to afford the title compound (5.3 g, 97.2% yield) as a colorless oil. LCMS calc. for C₉H₁₄NO₂ [M+H]⁺: m/z=168.1; Found: 168.2.

### Step 2: ethyl 4,5-dibromo-3-(bromomethyl)-1-methyl-1H-pyrrole-2-carboxylate

To a solution of ethyl 1,3-dimethyl-1H-pyrrole-2-carboxylate (1 g, 6 mmol) in CCl₄ (30 mL) was added N-bromosuccinimide (3.2 g, 18 mmol) and benzoyl peroxide (43 mg, 0.18 mmol). The mixture was heated at reflux for 2 h., then cooled to r.t. and filtered. The filtrate was concentrated. The residue was dissolved in EtOAc (30 mL) and washed with sat. NaHCO₃ aq. (20 mL×2). The combined aqueous layers were extracted with EtOAc (20 mL). The combined organic layers were dried over Na₂SO₄, filtered and concentrated to afford the title compound (crude, 2.6 g).

### Step 3: ethyl 4,5-dibromo-3-(((N-(2-methoxy-2-oxoethyl)-4-methylphenyl)sulfon amido)methyl)-1-methyl-1H-pyrrole-2-carboxylate

A mixture of ethyl 4,5-dibromo-3-(bromomethyl)-1-methyl-1H-pyrrole-2-carboxylate (2.6 g, 6.48 mmol), potassium carbonate (1 g, 7.46 mmol) and potassium iodide (60 mg, 0.36 mmol) in acetone (15 mL) was stirred at 60° C. for 30 min. To this mixture was added methyl tosylglycinate (1.8 g, 7.46 mmol). The resulting mixture was stirred at 60° C. for 16 h., and then cooled to r.t and filtered. The solid was washed with acetone (30 mL). The filtrate was concentrated. The resulting residue was dissolved in DCM (50 mL), washed with H₂O (20 mL×3), dried over Na₂SO₄, filtered and concentrated. The residue was purified by flash chromatography on a silica gel column eluting with EtOAc/PE (20% to 50%) to afford the title compound (2.4 g, 95% yield) as a yellow solid. ¹H NMR (400 MHZ, CDCl₃) δ ppm 7.60 (d, J=8.3 Hz, 2H), 7.21 (d, J=7.7 Hz, 2H), 4.60 (s, 2H), 4.25 (q, J=7.1 Hz, 2H), 3.83 (s, 2H), 3.83 (s, 3H), 3.46 (s, 3H), 2.35 (s, 3H), 1.25 (t, J=7.1 Hz, 3H).

### Step 4: methyl 2,3-dibromo-7-hydroxy-1-methyl-1H-pyrrolo[3,2-c]pyridine-6-carboxylate

To a stirred solution of ethyl 4,5-dibromo-3-(((N-(2-methoxy-2-oxoethyl)-4-methylphenyl)sulfonamido)methyl)-1-methyl-1H-pyrrole-2-carboxylate (1 g, 4.26 mmol) in THF (20 mL) was added LiHMDS (12.8 mL, 12.8 mmol, 1.0 M in THF) dropwise at −78° C. over 2 h. After addition, the resulting mixture was stirred at −78° C. for an additional 1 h., then quenched with saturated NH₄Cl aq. (20 mL). The resulting mixture was extracted with EtOAc (50 mL×3). The combined organic layers were washed with H₂O (20 mL×2). The aqueous layers were combined and extracted with additional EtOAc (50 mL×2). The organic layers were combined, washed with brine (50 mL), dried over Na₂SO₄, filtered and concentrated. The residue was purified by flash chromatography on a silica gel column eluting with EtOAc/PE (20% to 50%) to afford the title compound (1 g, 65% yield) as a yellow solid. ¹H NMR (400 MHZ, DMSO-d₆) δ ppm 11.58 (s, 1H), 8.24 (s, 1H), 4.09 (s, 3H), 3.95 (s, 3H). LCMS calc. for C₁₀H₉Br₂N₂O₃ [M+H]⁺: m/z=362.9; Found: 362.9.

### Step 5: methyl 7-hydroxy-1-methyl-1H-pyrrolo[3,2-c]pyridine-6-carboxylate

To a solution of methyl 2,3-dibromo-7-hydroxy-1-methyl-1H-pyrrolo[3,2-c]pyridine-6-carboxylate (1 g, 2.76 mmol) in MeOH (14 mL) was added Et₃N (1.9 mL, 13.81 mmol) and Pd/C (10% wt, 100 mg). The mixture was stirred at r.t. under H₂ atmosphere (balloon) for 1 h., filtered and concentrated in vacuo. The mixture was purified by flash chromatography on a silica gel column eluting with EtOAc/PE (20% to 50%) to afford the title compound (417 mg, 73% yield) as a yellow solid. LCMS calc. for C₁₀H₁₁N₂O₃ [M+H]⁺: m/z=207.1; Found: 207.2.

### Step 6: 7-hydroxy-1-methyl-1H-pyrrolo[3,2-c]pyridine-6-carboxylic acid

To a solution of methyl 7-hydroxy-1-methyl-1H-pyrrolo[3,2-c]pyridine-6-carboxylate (100 mg, 0.49 mmol) in THF (1.8 mL) was added LiOH (35 mg, 1.46 mmol) and H₂O (0.6 mL). The mixture was heated at 60° C. for 2 h. and concentrated in vacuo. The residue was diluted with water (5 mL) and acidified to pH=5-6 with HCl aq. (1 N). The precipitates were collected by filtration and azeotroped with MeOH (10×3) to remove water to provide the title compound (65 mg, 69% yield) as a white solid. ¹H NMR (400 MHZ, DMSO-d₆) δ ppm 8.36 (s, 1H), 7.68 (d, J=3.1 Hz, 1H), 6.87 (d, J=3.1 Hz, 1H), 4.21 (s, 3H). LCMS calc. for C₉H₉N₂O₃ [M+H]⁺: m/z=193.1; Found: 193.2.

### INT C43: 4-Hydroxy-7-methylpyrrolo[1,2-a]pyrimidine-3-carboxylic acid

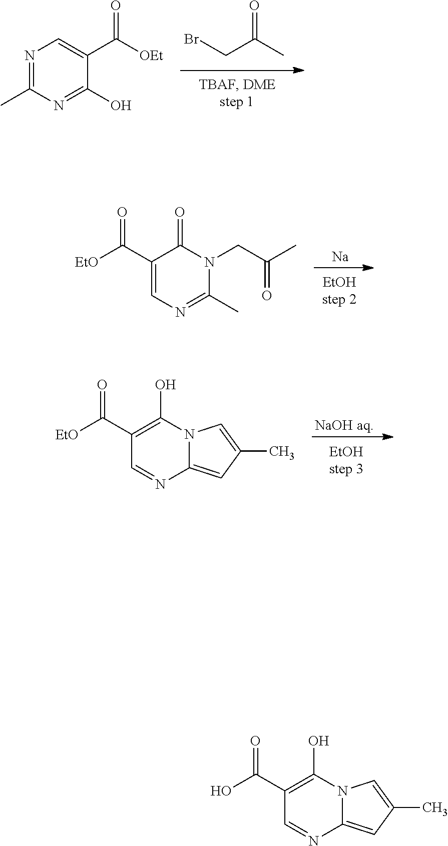

### Step 1: ethyl 2-methyl-6-oxo-1-(2-oxopropyl)-1,6-dihydropyrimidine-5-carboxylate

A mixture of ethyl 4-hydroxy-2-methylpyrimidine-5-carboxylate (2.0 g, 10.98 mmol), 1-bromopropan-2-one (3.0 g, 21.96 mmol) and TBAF (5.8 g, 21.96 mmol) in DME (40 mL) was stirred at r.t. under N₂ atmosphere overnight. The mixture was concentrated and the residue was purified by flash chromatography on a silica gel column eluting with MeOH/DCM (5%) to afford the title compound (2.0 g, 80% yield) as a yellow oil. LCMS calc. for C₁₁H₁₅N₂O₄ [M+H]⁺: m/z=239.1; Found: 239.1.

### Step 2: ethyl 4-hydroxy-7-methylpyrrolo[1,2-a]pyrimidine-3-carboxylate

A mixture of Na (184 mg, 8.0 mmol) in EtOH (10 mL) was stirred at r.t. under N₂ atmosphere for 1 h., ethyl 2-methyl-6-oxo-1-(2-oxopropyl)-1,6-dihydropyrimidine-5-carboxylate (953 mg, 4.0 mmol) in EtOH (5 mL) was added to the solution. The resulting mixture was stirred at r.t. for 1 h., and then acidified to pH=5-6 with HCl aq. (2 N). The solids formed were collected by filtration, and dried under vacuum to afford the title compound (500 mg, 50% yield) as a gray solid. LCMS calc. for C₁₁H₁₃N₂O₃ [M+H]⁺: m/z=221.1; Found: 221.1.

### Step 3: 4-hydroxy-7-methylpyrrolo[1,2-a]pyrimidine-3-carboxylic acid

A solution of ethyl 4-hydroxy-7-methylpyrrolo[1,2-a]pyrimidine-3-carboxylate (100 mg, 0.4 mmol) and NaOH aq. (8 N, 3 mL) in EtOH (5 mL) was stirred at 80° C. for 6 h. The reaction mixture was cooled to r.t. and acidified to pH=5-6 with HCl aq. (1 N). Solids were collected by filtration and dried to afford the title compound (50 mg, 50% yield) as a white solid. ¹H NMR (400 MHZ, DMSO-d₆) δ ppm 13.37 (s, 1H), 8.50 (s, 1H), 7.28 (dd, J=1.9, 1.1 Hz, 1H), 6.13 (d, J=1.8 Hz, 1H), 2.19 (d, J=1.0 Hz, 3H). LCMS calc. for C₉H₉N₂O₃ [M+H]⁺: m/z=193.1; Found: 193.2.

### INT C44: 4-Hydroxy-1-methyl-1H-pyrrolo[2,3-c]pyridine-5-carboxylic acid

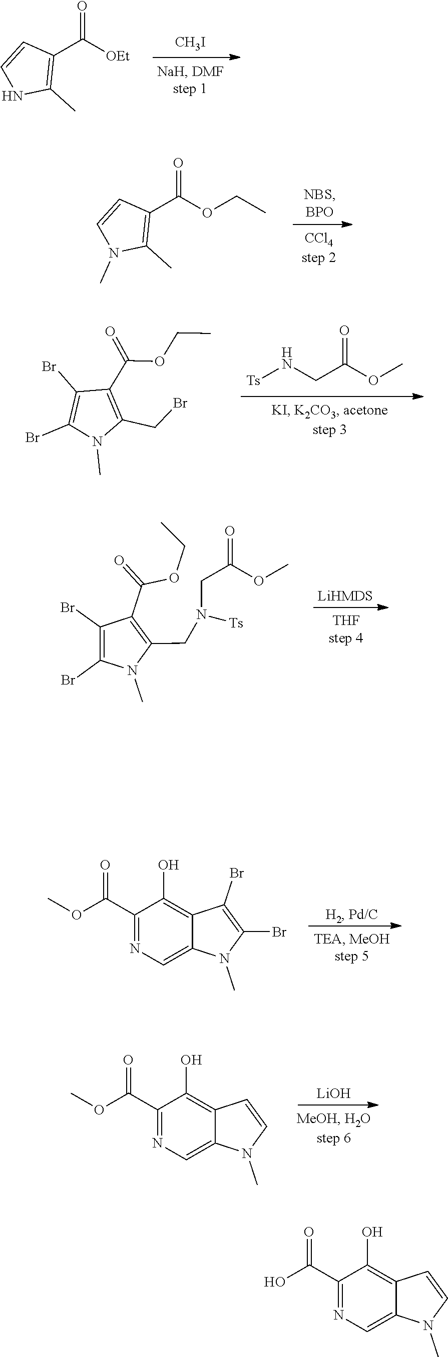

This compound was prepared using procedures analogous to those described for INT C42 step 1-6 using ethyl 2-methyl-1H-pyrrole-3-carboxylate to replace ethyl 3-methyl-1H-pyrrole-2-carboxylate in step 1 to afford the title product as a white solid. ¹H NMR (400 MHZ, DMSO-d₆) δ ppm 8.17 (s, 1H), 7.44 (d, J=3.0 Hz, 1H), 6.56 (d, J=2.5 Hz, 1H), 3.88 (s, 3H). LCMS calc. for C₉H₉N₂O₃ [M+H]⁺: m/z=193.1; Found: 193.1.

### INT C45: 4-Hydroxypyrazolo[1,5-a]pyridine-5-carboxylic acid

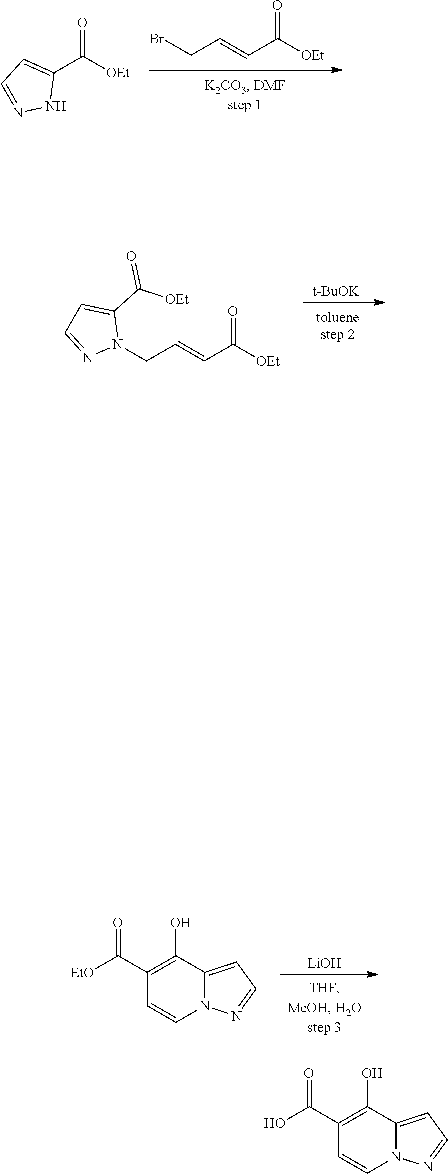

### Step 1: ethyl 1-(4-ethoxy-4-oxobut-2-en-1-yl)-1H-pyrazole-5-carboxylate

To a solution of ethyl 1H-pyrazole-5-carboxylate (400 mg, 2.86 mmol) and K₂CO₃ (1.2 g, 8.58 mmol) in DMF (20 mL) was added ethyl 4-bromobut-2-enoate (660 mg, 3.42 mmol) at r.t. The mixture was stirred at r.t. for 16 h., diluted with H₂O (20 mL) and extracted with EtOAc (20 mL×3). The combined organic layers were washed with brine (20 mL), dried over Na₂SO₄, filtered and concentrated under reduced pressure to afford the title compound (700 mg, crude) as a brown oil, which was used in the next step without further purification. LCMS calc. for C₁₂H₁₇N₂O₄ [M+H]⁺: m/z=253.1; Found: 253.3.

### Step 2: ethyl 4-hydroxypyrazolo[1,5-a]pyridine-5-carboxylate

To a solution of ethyl 1-(4-ethoxy-4-oxobut-2-en-1-yl)-1H-pyrazole-5-carboxylate (700 mg, 2.78 mmol) in toluene (5 mL) was added t-BuOK (883 mg, 4.17 mmol) at r.t. under N₂. The mixture was stirred at 110° C. for 1 h. under N₂. The mixture was cooled to r.t., acidified with HCl aq. (1 N, 5 mL) and extracted with EtOAc (20 mL×3). The combined organic layers were dried over Na₂SO₄, filtered, concentrated under reduced pressure. The residue was purified by flash chromatography on a C18 column eluting with MeCN/H₂O (5-50%, with 0.1% FA) to afford the title compound (150 mg, 26% yield) as a light yellow solid. ¹H NMR (400 MHZ, DMSO-d₆) δ ppm 11.57 (s, 1H), 8.27 (dd, J=7.2, 0.8 Hz, 1H), 8.06 (d, J=2.4 Hz, 1H), 7.06 (d, J=7.2 Hz, 1H), 6.99 (dd, J=2.4, 0.8 Hz, 1H), 4.40 (q, J=7.2 Hz, 2H), 1.37 (t, J=7.2 Hz, 3H).LCMS calc. for C₁₀H₁₁N₂O₃ [M+H]⁺: m/z=207.1; Found: 207.2.

### Step 3: 4-hydroxypyrazolo[1,5-a]pyridine-5-carboxylic acid

To a solution of ethyl 4-hydroxypyrazolo[1,5-a]pyridine-5-carboxylate (150 mg, 0.73 mmol) in THF (6 mL), MeOH (2 mL) and H₂O (2 mL) was added LiOH (175 mg, 7.28 mmol) at r.t. The mixture was stirred at 60° C. for 16 h., cooled to r.t. and acidified to pH=5-6 with HCl aq. (1 N). The solid was collected by filtration, washed with H₂O (5.0 mL) and dried under reduced pressure to afford the title product (90 mg, 69% yield) as a light brown solid. LCMS calc. for C₈H₇N₂O₃ [M+H]⁺: m/z=179.1; Found: 179.0.

### INT C46: 2-(Methoxyamino)-2-oxoacetic acid

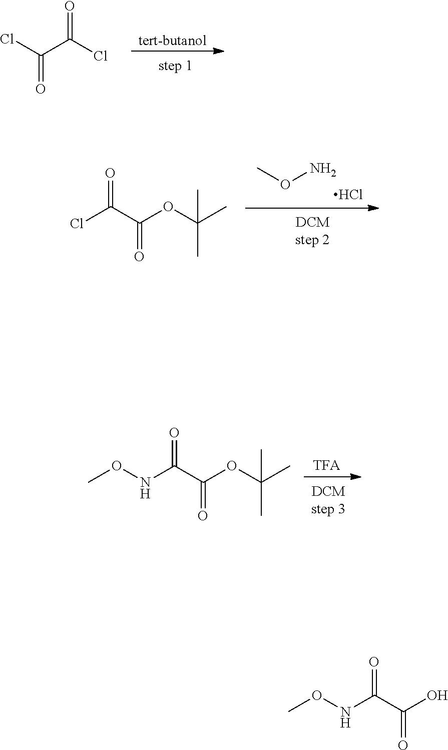

### Step 1: tert-butyl 2-chloro-2-oxoacetate

A three necked flask was dried, flushed with nitrogen, sealed and cooled in ice for 2 min., then oxalyl chloride (8.7 ml, 100 mmol) was injected and cooled for 10 min. To the flask was added tert-butanol (4.8 ml, 50 mmol) dropwise over 20 min. The reaction flask was wrapped in aluminum foil and the mixture was stirred in an ice bath for 15 min. and then left to reach r.t. over 45 min. The excess oxalyl chloride was removed by evaporation to afford the title compound (6.4 g, 77% yield) as a colorless liquid.

### Step 2: tert-butyl 2-(methoxyamino)-2-oxoacetate

A mixture of o-methylhydroxylamine hydrochloride (506 mg, 6.08 mmol) and TEA (1.54 g, 12.19 mmol) in DCM (20 mL) was stirred at 0° C. for 10 min., tert-butyl 2-chloro-2-oxoacetate (500 mg, 3.04 mmol) was then added dropwise over 2 min. The mixture was stirred at 4° C. for 2 h., diluted with water (100 mL), extracted with DCM (50 mL×3). The combined organic layers were dried over anhydrous Na₂SO₄, filtered and concentrated under reduced pressure. The residue was purified by flash chromatography on a silica gel column eluting with MeOH/DCM (0-5%) to afford the title compound (400 mg, 37%) as a colorless oil. ¹H NMR (400 MHZ, CDCl₃) δ ppm 9.43 (s, 1H), 3.83 (s, 3H), 1.55 (s, 9H).

### Step 3: 2-(methoxyamino)-2-oxoacetic acid

A solution of tert-butyl 2-(methoxyamino)-2-oxoacetate (100 mg, 0.57 mmol) in DCM/TFA (2:1, 3 mL) was stirred at 0° C. for 30 min. The reaction mixture was concentrated to afford the title compound which was directly used in next step without further purification. ¹H NMR (400 MHZ, DMSO-d₆) δ ppm 12.19 (s, 1H), 3.63 (s, 3H).

### INT C47: 4-Hydroxyimidazo[1,5-b]pyridazine-3-carboxylic acid

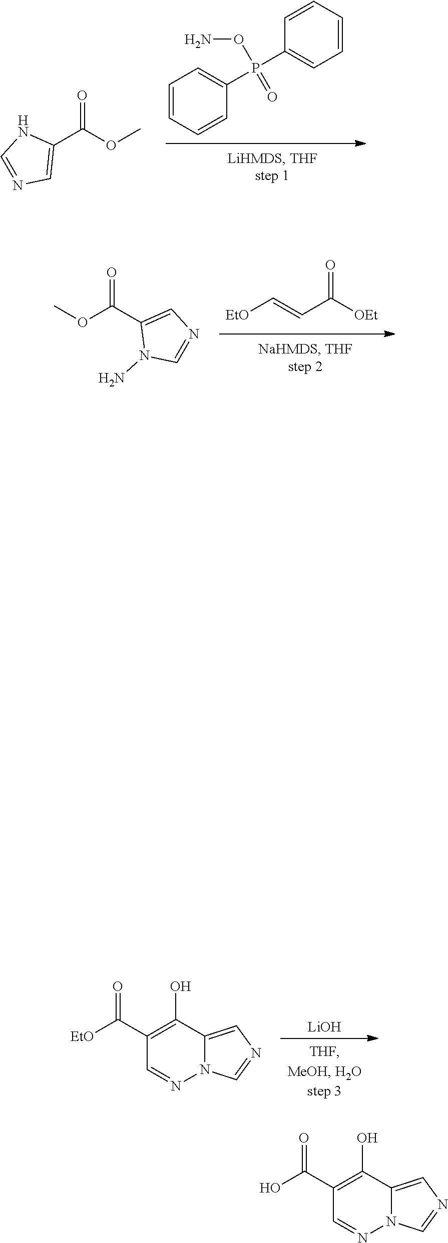

### Step 1: methyl 1-amino-1H-imidazole-5-carboxylate

To a solution of methyl 1H-imidazole-5-carboxylate (1.0 g, 7.94 mmol) in THF (20 mL) was added LiHMDS (9.5 mL, 1 M in THF, 9.53 mmol) dropwise over 30 min. at −78° C. Then the mixture was stirred at −78° C. for 2 h. and allowed to warm to −10° C. (aminooxy)diphenylphosphine oxide (2.0 g, 8.73 mmol) was added at this temperature. Then the mixture was stirred at r.t. for 16 h., quenched with H₂O (20 mL) and concentrated. The residue was purified by flash chromatography on a silica gel column eluting with MeOH/DCM (5%) to afford the title compound (480 mg, 43% yield) as a white solid. ¹H NMR (400 MHZ, DMSO-d₆) δ ppm 7.82 (s, 1H), 7.54 (d, J=1.2 Hz, 1H), 6.20 (s, 2H), 3.79 (s, 3H). LCMS calc. for C₅H₈N₃O₂ [M+H]⁺: m/z=142.1; Found: 142.0.

### Step 2: ethyl 4-hydroxyimidazo[1,5-b]pyridazine-3-carboxylate

To a solution of methyl 1-amino-1H-imidazole-5-carboxylate (300 mg, 2.13 mmol) and ethyl (E)-3-ethoxyacrylate (337 mg, 2.34 mmol) in THF (5 mL) was added NaHMDS (2.6 mL, 1 M in THF, 2.55 mmol) dropwise at −78° C. The solution was warmed to r.t., stirred for 1 h. and concentrated under reduced pressure. The residue was purified by flash chromatography on a C18 column eluting with MeCN/H₂O (0%-10%, with 0.1% FA) to afford the title compound (45 mg, 10% yield) as a white solid. LCMS calc. for C₉H₁₀N₃O₃ [M+H]⁺: m/z=208.1; Found: 208.0.

### Step 3: 4-hydroxyimidazo[1,5-b]pyridazine-3-carboxylic acid

To a solution of ethyl 4-hydroxyimidazo[1,5-b]pyridazine-3-carboxylate (45 mg, 0.22 mmol) in THF (3 mL), MeOH (1 mL) and H₂O (1 mL) was added LiOH (52 mg, 2.17 mmol) at r.t. The mixture was stirred at 60° C. for 8 h., cooled to r.t. and then adjusted to pH=5-6 with HCl aq. (1 N). The precipitates formed were collected by filtration, washed with H₂O (5.0 mL) and dried under vacuum to afford the title compound (22 mg, 57% yield) as a light brown solid. LCMS calc. for C₇H₆N₃O₃ [M+H]⁺: m/z=180.0; Found: 179.9.

### INT C48: 2-((5-Methyl-1H-pyrazol-3-yl)amino)-2-oxoacetic acid

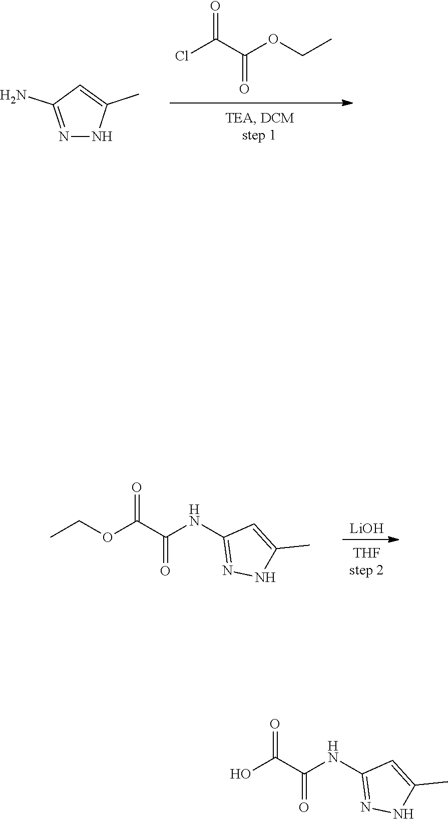

### Step 1: ethyl 2-((5-methyl-1H-pyrazol-3-yl)amino)-2-oxoacetate

To a stirred solution of 5-methyl-1H-pyrazol-3-amine (500 mg, 5.15 mmol) and TEA (1.56 g, 15.4 mmol) in DCM (100 mL) was added ethyl 2-chloro-2-oxoacetate (843 mg, 6.18 mmol) at 0° C. The mixture was stirred at r.t. for 3 h., then poured into water (30 mL), and extracted with DCM (30 mL×3). The combined organic layers were washed with brine (10 mL), dried over Na₂SO₄, filtered, and concentrated under reduced pressure. The residue was purified by flash chromatography on a silica gel column eluting with MeOH/DCM (5%) to afford the title compound (460 mg, 45% yield) as a white solid. ¹HNMR (400 MHZ, DMSO-d₆) δ ppm 12.24 (s, 1H), 10.88 (s, 1H), 6.27 (s, 1H), 4.26 (q, J=7.1 Hz, 2H), 2.21 (s, 3H), 1.29 (t, J=7.1 Hz, 3H). LCMS calc. for C₈H₁₂N₃O₃ [M+H]⁺: m/z=198.1; Found: 198.6.

### Step 2: 2-((5-methyl-1H-pyrazol-3-yl)amino)-2-oxoacetic acid

To a stirred solution of ethyl 2-((5-methyl-1H-pyrazol-3-yl)amino)-2-oxoacetate (100 mg, 0.51 mmol) in THF (2 mL) was added LiOH aq. (2 N, 0.7 mL) at 0° C. The mixture was stirred at 0° C. for 2 h. and concentrated under reduced pressure. The residue was acidified using HCl aq. (1 N) until pH=5 to 6. The solids were collected by filtration and dried under vacuum to afford the title compound (74 mg, 86% yield) as a white solid. ¹HNMR (400 MHZ, DMSO-d₆) δ ppm 13.07 (s, 1H), 10.67 (s, 1H), 6.26 (s, 1H), 2.21 (s, 3H). LCMS calc. for C₆H₈N₃O₃ [M+H]⁺: m/z=170.0; Found: 170.2.

### INT C49: 2-Oxo-2-(thiazol-5-ylamino)acetic acid

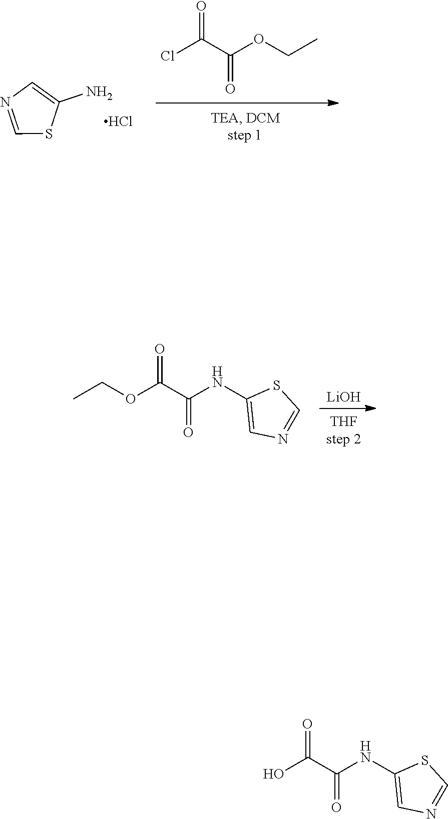

This compound was prepared using procedures analogous to those described for INT C48 step 1-2 using thiazol-5-amine hydrochloride to replace 5-methyl-1H-pyrazol-3-amine in Step 1 to afford the title product as a white solid. ¹H NMR (400 MHZ, DMSO-d₆) δ ppm 12.29 (s, 1H), 8.69 (s, 1H), 7.89 (d, J=0.9 Hz, 1H). LCMS calc. for C₅H₅N₂O₃S [M+H]⁺: m/z=173.0; Found: 173.1.

### INT C50: 4-Hydroxyisoquinoline-3-carboxylic acid

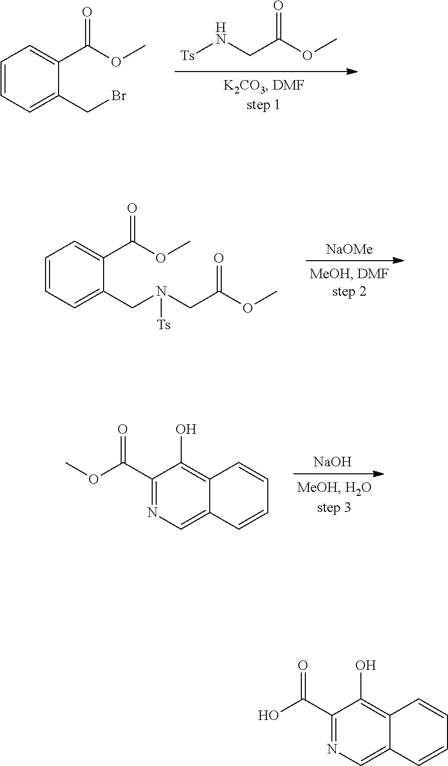

### Step 1: methyl 2-(((N-(2-methoxy-2-oxoethyl)-4-methylphenyl)sulfonamido)methyl)benzoate

To a solution of methyl 2-(bromomethyl)benzoate (3.0 g, 13.10 mmol) and methyl tosylglycinate (3.54 g, 14.54 mmol) in DMF (30 mL) was added K₂CO₃ (2.75 g, 19.78 mmol) and NaI (2.96 g, 19.78 mmol). The mixture was stirred at 60° C. for 16 h., then diluted with H₂O (100 mL) and extracted with EtOAc (100 mL×3). The combined organic layers were washed with brine (100 mL), saturated NH₄Cl aq. (100 mL), dried over Na₂SO₄, filtered and concentrated. The residue was purified by flash chromatography on a silica gel column eluting with EtOAc/PE (70%) to afford the title compound (2.02 g, 39% yield) as a yellow oil. LCMS calc. for C₁₉H₂₂NO₆S [M+H]⁺: m/z=392.1; Found: 392.6.

### Step 2: methyl 4-hydroxyisoquinoline-3-carboxylate

A solution of methyl 2-(((N-(2-methoxy-2-oxoethyl)-4-methylphenyl)sulfonamido)methyl)benzoate (2.08 g, 5.31 mmol) in 30% NaOMe methanol solution (10 mL) and DMF (20 mL) was stirred at r.t. for 2 h., then diluted with H₂O (100 mL), extracted with EtOAc (100 mL×3). The combined organic layers were washed with brine (100 mL), saturated NH₄Cl aq. (100 mL), dried over Na₂SO₄, filtered and concentrated. The residue was purified by Prep-HPLC on a C18 column eluting with MeOH/H₂O (5-95%, with 0.1% NH₄HCO₃) to afford the title compound (463 mg, 43% yield) as a yellow solid. LCMS calc. for C₁₁H₁₀NO₃ [M+H]⁺: m/z=204.1; Found: 204.0.

### Step 3: 4-hydroxyisoquinoline-3-carboxylic acid

To a solution of methyl 4-hydroxyisoquinoline-3-carboxylate (363 mg, 1.79 mmol) in MeOH (8 mL) and H₂O (0.8 mL) was added NaOH (285.8 mg, 7.15 mmol). The mixture was stirred at 50° C. for 16 h., then concentrated, diluted with water (25 mL) and extracted with EtOAc (50 mL×3). The aqueous layer was adjusted to pH to 2-3 with HCl aq. (6 N). The precipitate was collected by filtration. The filtrate was concentrated and purified by Prep-HPLC on a C18 column eluting with MeOH/H₂O (5%-95%, with 0.1% FA) to afford the title compound (combined with the solids collected: 153 mg, 45.3% yield) as a yellow solid. ¹H NMR (400 MHZ, DMSO-d₆) δ ppm 8.83 (s, 1H), 8.41 (dd, J=14.7, 8.1 Hz, 2H), 8.07-8.01 (m, 1H), 7.99-7.94 (m, 1H). LCMS calc. for C₁₀H₈NO₃ [M+H]⁺: m/z=190.0; Found: 190.0.

### INT C51: 2,5-Difluorobenzoyl Isocyanate

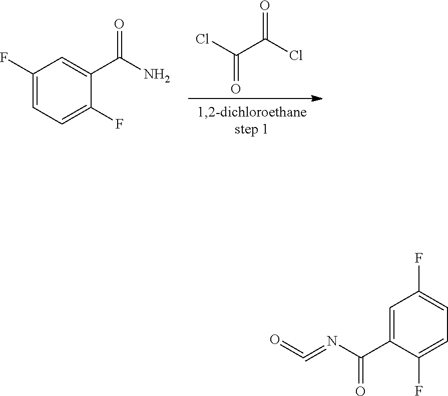 [SMILES: NC(=O)c1cc(F)ccc1F ; O=C(Cl)C(=O)Cl ; O=C=NC(=O)c1cc(F)ccc1F]

A mixture of 2,5-difluorobenzamide (1.0 g, 6.36 mmol), oxalyl dichloride (1.2 g mg, 9.55 mmol) in 1,2-dichloroethane (0.94 g, 9.55 mmol) was stirred at 84° C. for 5 h. The reaction mixture was concentrated to afford the title compound (1.16 g) as a grey solid which was used for the next step without further purification.

### INT C52: 5-Oxo-4,5-dihydrothieno[3,2-b]pyridine-6-carboxylic acid

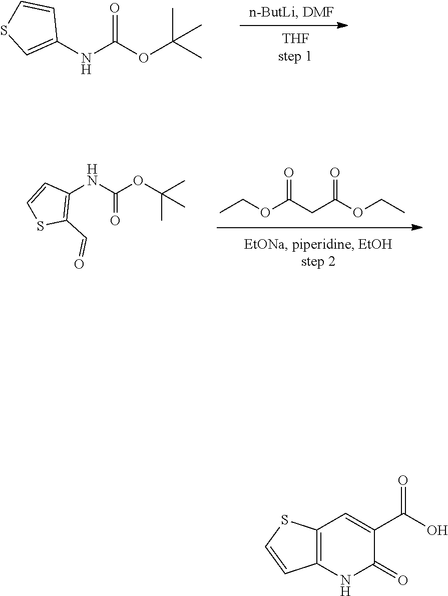

### Step 1: tert-butyl (2-formylthiophen-3-yl)carbamate

Tert-butyl thiophen-3-ylcarbamate (5.0 g, 20 mmol) was dissolved in THF (100 mL) at r.t. under nitrogen and cooled to −78° C. To the cooled solution was added n-butyl lithium (2.5 M in hexanes, 20 mL, 50 mmol) dropwise. After completion of addition, the mixture was stirred at −78° C. for 1 h, and then DMF (2.8 g, 40 mmol) was added. The mixture was stirred at r.t. until completion (˜1 h.), and quenched with MeOH. The mixture was concentrated. The residue was purified by flash chromatography on a silica gel column eluting with EA/PE (5%) to afford the title compound (4.5 g, 79% yield) as a yellow oil. ¹H NMR (400 MHZ, DMSO-d₆) δ ppm 10.13 (s, 1H), 9.94 (d, J=0.9 Hz, 1H), 8.01 (dt, J=5.4, 0.6 Hz, 1H), 7.60 (d, J=5.4 Hz, 1H), 1.49 (s, 9H). LCMS calc. for C₁₀H₁₄NO₃S [M+H]⁺: m/z=228.1; Found: [M+H−56]⁺: 172.2.

### Step 2: 5-oxo-4,5-dihydro thieno[3,2-b]pyridine-6-carboxylic acid

A mixture of tert-butyl (2-formylthiophen-3-yl)carbamate (4.5 g, 20 mmol), diethyl malonate (6.4 g, 40 mmol), EtONa (13.6 g, 200 mmol) and piperidine (1.7 g, 20 mmol) in EtOH (100 mL) was stirred at 85° C. under N₂ atmosphere for 2 h. The mixture was concentrated. The residue was diluted with water and washed with DCM (100 mL×3). The aqueous layer was adjusted to pH to 4-5 with HCl aq. (6 N). The precipitate was filtered and dried to afford the title product (1.6 g) as a yellow solid. ¹H NMR (400 MHZ, DMSO-d₆) δ ppm 15.15 (s, 1H), 9.05 (s, 1H), 8.33 (d, J=5.5 Hz, 1H), 7.21 (d, J=5.4 Hz, 1H). LCMS calc. for C₈H₆NO₃S [M+H]⁺: m/z=196.0; Found: 196.1.

### INT C53: 7-Chloro-2-hydroxypyrazolo[1,5-a]pyridine-3-carboxylic acid

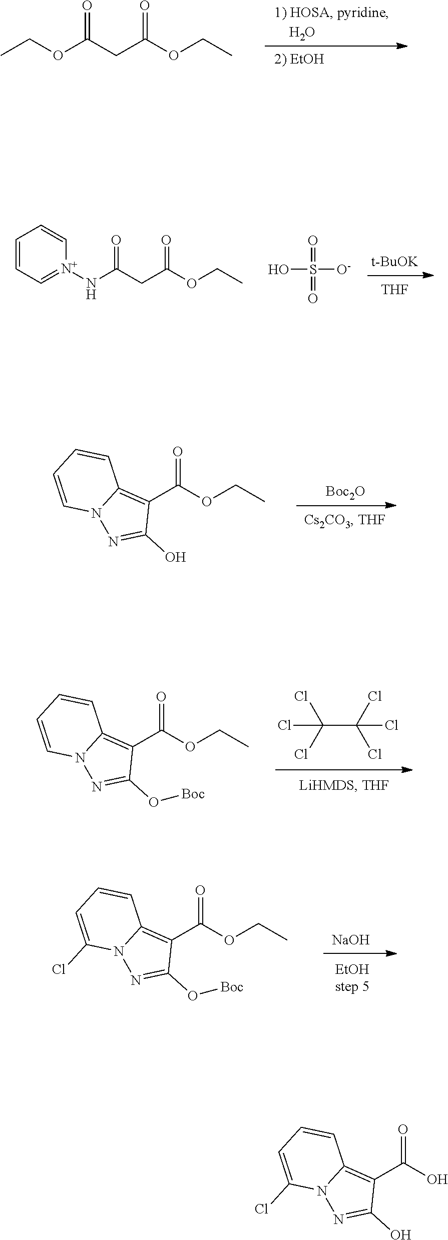

### Step 1: ethyl 3-oxo-3-(pyridin-1-ium-1-ylamino)propanoate hydrogen sulfate

A mixture of hydroxylamine-O-sulfonic acid (HOSA, 10 g, 88.5 mmol) and pyridine (21 g, 265.5 mmol) in water (200 mL) was heated at 90° C. for 1 h. After it was cooled to r.t., K₂CO₃ (12 g, 88.5 mmol) was added to the mixture, and stirred at r.t. for 30 mins. The resulting suspension was concentrated under vacuum to afford 1-aminopyridin-1-ium hydrogen sulfate (crude).

The crude 1-aminopyridin-1-ium hydrogen sulfate was taken up with absolute ethanol (200 mL). The resulting mixture was filtered. The filtrate was mixed with diethyl malonate (28 g, 177.0 mmol) and stirred at 90° C. for 16 h. The mixture was concentrated under vacuum. The residue was purified by flash chromatography on a silica gel column eluting with MeOH/DCM (10%) to afford the title compound (13 g, 72.2% yield) as a brownish sticky oil. LCMS calc. for C₁₀H₁₃N₂O₃⁺ [M]⁺: m/z=209.1; Found: 209.2.

### Step 2: ethyl 2-hydroxypyrazolo[1,5-a]pyridine-3-carboxylate

To a solution of ethyl 3-oxo-3-(pyridin-1-ium-1-ylamino)propanoate hydrogen sulfate (13 g) in dry THF (150 mL) was added potassium tert-butoxide (7 g, 62.5 mmol) in portions. The resulting dark-orange suspension was stirred at r.t. for 10 min., and concentrated under vacuum. The residue was diluted with water and acidified to pH 2 with HCl aq. (0.5 N, 50 mL), and extracted with EtOAc (150 mL×2). The combined organic layers were dried over Na₂SO₄, filtered and evaporated under vacuum. The residue was purified by flash chromatography on a silica gel column eluting with MeOH/DCM (10%) to afford the desired compound (5.5 g, 42.3% yield) as a white solid. LCMS calc. for C₁₀H₁₁N₂O₃ [M+H]⁺: m/z=207.1; Found: 207.0.

### Step 3: ethyl 2-hydroxypyrazolo[1,5-a]pyridine-3-carboxylate ethyl 2-((tert-butoxy carbonyl)oxy)pyrazolo[1,5-a]pyridine-3-carboxylate

Cs₂CO₃ (16.7 g, 51.0 mmol) and tert-butoxycarbonyl anhydride (4.1 g, 18.69 mmol) were added to a solution of ethyl 2-hydroxypyrazolo[1,5-a]pyridine-3-carboxylate (3.5 g, 17.0 mmol) in dry THF (85 mL). The mixture was stirred under reflux overnight and cooled to r.t. The solvent was concentrated under reduced pressure. The residue was dissolved in water (100 mL) and extracted with diethyl ether (200 mL×3). The combined organic layers were washed with brine, dried over anhydrous Na₂SO₄, filtered and concentrated under reduced pressure. The residue was purified by flash chromatography on a silica gel column eluting with EtOAc/PE (20%) to afford the title compound (4.9 g, 94.2% yield) as a white solid. ¹H NMR (400 MHZ, CDCl₃) δ ppm 8.39 (d, J=6.9 Hz, 1H), 8.09 (d, J=8.9 Hz, 1H), 7.46-7.41 (m, 1H), 6.97 (t, J=7.0 Hz, 1H), 4.37 (q, J=7.1 Hz, 2H), 1.59 (s, 9H), 1.40 (t, J=7.1 Hz, 3H). LCMS calc. for C₁₅H₁₉N₂O₅ [M+H]⁺: m/z=307.1; Found: [M+H−100]⁺: 207.1.

### Step 4: ethyl 2-((tert-butoxycarbonyl)oxy)-7-chloropyrazolo[1,5-a]pyridine-3-carboxylate

LiHMDS (1.0 M THF solution: 3.0 mL, 3.04 mmol) was added dropwise to a solution of ethyl 2-((tert-butoxycarbonyl)oxy)pyrazolo[1,5-a]pyridine-3-carboxylate (620 mg, 2.03 mmol) in dry THF (10 mL) at −78° C. Then the mixture was stirred at −78° C. for 1 h., a solution of hexachloroethane (529 mg, 2.23 mmol) in dry THF (5 mL) was added at −78° C. The mixture was stirred for 15 min. at r.t. Subsequently, the reaction was quenched with sat. NH₄Cl aq. (50 mL). The aqueous layer was extracted with DCM (50 mL×4). The combined organic layers were dried over Na₂SO₄, filtered and evaporated to dryness under vacuum. The residue was purified by Prep-HPLC on a C18 column eluting with MeCN/H₂O (5-95%, with 0.1% NH₄HCO₃) to afford the title compound (1.0 g, 45% yield) as a white solid. LCMS calc. for C₁₅H₁₈ClN₂O₅ [M+H]⁺: m/z=341.1; Found: [M+H−100]⁺: 241.6.

### Step 5: 7-chloro-2-hydroxypyrazolo[1,5-a]pyridine-3-carboxylic acid

NaOH aq. (0.25 mL, 6 M) was added to a solution of ethyl 2-((tert-butoxycarbonyl)oxy)-7-chloropyrazolo[1,5-a]pyridine-3-carboxylate (50 mg, 0.15 mmol) in EtOH (2 mL). The mixture was stirred for 4 h. at 75° C. The reaction mixture was neutralized with HCl (6 M) to PH˜6-7 and concentrated under reduced pressure. The residue was diluted with water and cooled to 0° C., then acidified with 2 M HCl until pH 2. The precipitate was collected and dried to afford the title compound (25 mg, 79% yield) as a white solid. LCMS calc. for C₈H₆ClN₂O₃ [M+H]⁺: m/z=213.0; Found: 213.0.

### INT C54: 8-Hydroxyimidazo[1,2-b]pyridazine-7-carboxylic acid

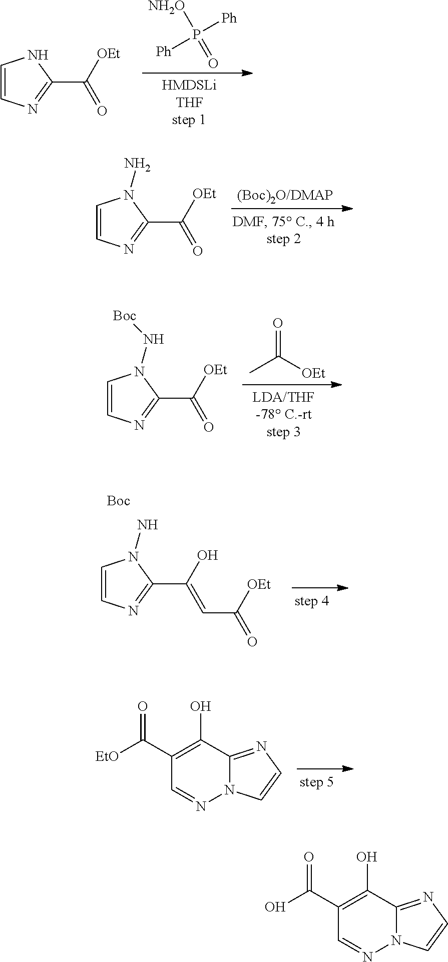

### Step 1: ethyl 1-amino-1H-imidazole-2-carboxylate

To a solution of ethyl 1H-imidazole-2-carboxylate (10 g, 71.4 mmol) in anhydrous DMF (200 mL) at −10° C. was added dropwise lithium hexamethyldisilazane (71 mL, 1 M solution in THF, 71.4 mmol). After the mixture was stirred for 10 min., (aminooxy)diphenylphosphine oxide (21.6 g, 92.9 mmol) was added at −10° C., followed by stirring at r.t. overnight. The reaction was quenched with water until a clear solution was formed and concentrated in vacuum to dryness. The residue was washed with EtOAc (100 mL) and filtered. The filtrate was concentrated in vacuo to give the title product (8 g, 72%) as a light yellow oil which was used in next step without further purification. LCMS calc. for C₆H₁₀N₃O₂ [M+H]⁺: m/z=156.1; Found: 156.2.

### Step 2: ethyl 1-((tert-butoxycarbonyl)amino)-1H-imidazole-2-carboxylate

To a stirred solution of ethyl 1-amino-1H-imidazole-2-carboxylate (8 g, 51.6 mmol) in DMF (100 mL) was added (Boc) 20 (10.1 g, 46.4 mmol), DMAP (944.5 mg, 7.7 mmol). The mixture was stirred at 85° C. for 4 h., poured into water and extracted with EtOAc (100 mL×3). The combined organic layers were washed with brine (100 mL), dried over Na₂SO₄, filtered and concentrated under reduced pressure. The residue was purified by flash chromatography on a silica gel column eluting with EtOAc/PE (20%) to afford the title product (6 g, 46%) as a white solid. LCMS calc. for C₁₁H₁₈N₃O₄ [M+H]⁺: m/z=256.1; Found: 256.4.

### Step 3: ethyl 3-(1-((tert-butoxycarbonyl)amino)-1H-imidazol-2-yl)-3-hydroxyacrylate

To a stirred solution of ethyl 1-((tert-butoxycarbonyl)amino)-1H-imidazole-2-carboxylate (6.0 g, 23.5 mmol) in THF (30 mL) was added LDA (70.3 mL, 2 M solution in THF, 141.2 mmol). The mixture was stirred at −78° C. for 10 min., then ethyl acetate (6.2 g, 70.3 mmol) was added. The resulting mixture was stirred at −78° C. for 40 min., quenched with NH₄Cl (60 mL) and extracted with EtOAc (50 mL×3). The combined organic layers were dried over Na₂SO₄, filtered and concentrated under reduced pressure. The residue was purified by flash chromatography on a silica gel column eluting with MeOH/DCM (15%) to afford the title product (6 g, 26.5%) as a yellow oil. LCMS calc. for C₁₃H₂₀N₃O₅ [M+H]⁺: m/z=298.1; Found: 298.1.

### Step 4: ethyl 8-hydroxyimidazo[1,2-b]pyridazine-7-carboxylate

To a solution of ethyl 3-(1-((tert-butoxycarbonyl)amino)-1H-imidazol-2-yl)-3-hydroxyacrylate (6 g, 20.2 mmol) in DCM (50 mL) was added DMF-DMA (12.02 g, 101 mmol). The reaction was stirred at r.t. overnight and concentrated. The residue was purified by flash chromatography on a silica gel column eluting with MeOH/DCM (25%) to afford the title product (1.0 g, 25%) as a yellow oil. LCMS calc. for C₉H₁₀N₃O₃ [M+H]⁺: m/z=208.1; Found: 208.2.

### Step 5:8-hydroxyimidazo[1,2-b]pyridazine-7-carboxylic acid

To a solution of ethyl 8-hydroxyimidazo[1,2-b]pyridazine-7-carboxylate (400 mg, 1.9 mmol) in EtOH (3 mL) and H₂O (1 mL) was added NaOH (309 mg, 7.7 mmol). The mixture was stirred at 90° C. for 1 h. The reaction mixture was concentrated and adjusted to pH with saturated citric acid and the precipitate was filtered to give the title product (100 mg, 28%) as a white solid. LCMS calc. for C₇H₆N₃₀ [M+H]⁺: m/z=180.0; Found: 180.1.

### INT C55: 2-Oxo-2-(thiazol-4-ylamino)acetic acid

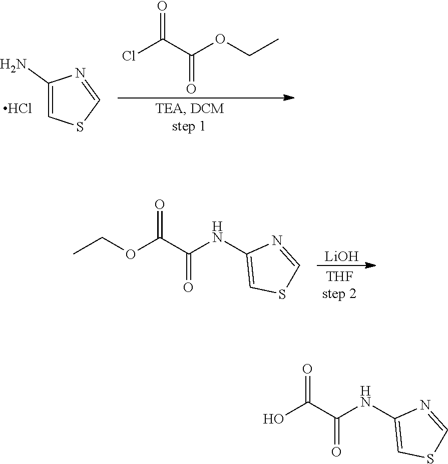

This compound was prepared using procedures analogous to those described for INT C48 step 1-2 using thiazol-4-amine hydrochloride to replace 5-methyl-1H-pyrazol-3-amine in step 1 to afford the title product as a white solid. ¹H NMR (400 MHZ, DMSO-d₆) δ ppm 14.24 (s, 1H), 11.38 (s, 1H), 9.03 (d, J=2.2 Hz, 1H), 7.74 (d, J=2.3 Hz, 1H). LCMS calc. for C₅H₅N₂O₃S [M+H]⁺: m/z=173.0; Found: 173.1.

### INT C56: 4-Hydroxypyrrolo[1,2-c]pyrimidine-3-carboxylic acid

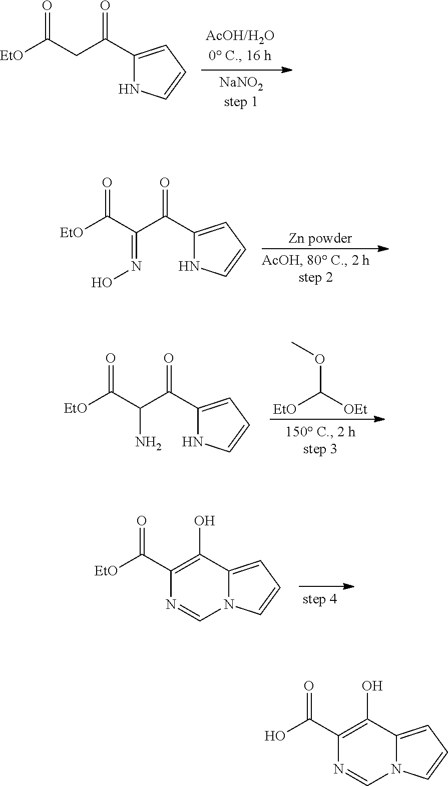

### Step 1: ethyl 2-(hydroxyimino)-3-oxo-3-(1H-pyrrol-2-yl)propanoate

To a solution of ethyl 3-oxo-3-(1H-pyrrol-2-yl)propanoate (50 mg, 0.28 mmol) in AcOH (1.5 mL) was added aq. NaNO₂ (25 mg, 0.36 mmol) in water (1.5 mL) at 0° C. The mixture was stirred at 0° C. overnight, and adjusted to pH ˜7-8 with sat. aq. NaHCO₃. The mixture was diluted with brine and extracted with EtOAc (30 mL×3). The combined organic layers were dried over anhydrous Na₂SO₄, filtered and concentrated to afford the title product (60 mg) as a yellow oil without further purification. LCMS calc. for C₉H₁₁N₂O₄ [M+H]⁺: m/z=211.1; Found: 211.0.

### Step 2: ethyl 2-amino-3-oxo-3-(1H-pyrrol-2-yl)propanoate

To a solution of ethyl 2-(hydroxyimino)-3-oxo-3-(1H-pyrrol-2-yl)propanoate (60 mg, 0.29 mmol) in AcOH (2 mL) was added Zn powder (56 mg, 0.87 mmol). The mixture was stirred at 80° C. for 2 h., and filtered. The filtrate was concentrated in vacuo to afford the title product (50 mg) as a brown oil which was used directly in the next step. LCMS calc. for C₉H₁₃N₂O₃ [M+H]⁺: m/z=197.1; Found: 197.0.

### Step 3: ethyl 4-hydroxypyrrolo[1,2-c]pyrimidine-3-carboxylate

A solution of ethyl 2-amino-3-oxo-3-(1H-pyrrol-2-yl)propanoate (50 mg, 0.26 mmol) in triethyl orthoformate (2 mL) was stirred at 150° C. for 2 h., and concentrated in vacuo. The residue was purified by flash chromatography on silica gel eluting with EtOAc/PE (20-50%) to afford the title compound (21 mg, 40%) as a white solid. ¹H NMR (400 MHZ, DMSO-d₆) δ ppm 11.63 (s, 1H), 8.39 (s, 1H), 7.19-7.09 (m, 1H), 7.02-6.98 (m, 1H), 6.28 (dd, J=6.1, 2.4 Hz, 1H), 4.35 (q, J=7.1 Hz, 2H), 1.33 (t, J=7.1 Hz, 3H). LCMS calc. for C₁₀H₁₁N₂O₃ [M+H]⁺: m/z=207.1; Found: 207.0.

### Step 4: 4-hydroxypyrrolo[1,2-c]pyrimidine-3-carboxylic acid

A mixture of ethyl 4-hydroxypyrrolo[1,2-c]pyrimidine-3-carboxylate (21 mg, 0.10 mmol) and lithium hydroxide (7 mg, 0.30 mmol) in methanol (0.9 mL) and water (0.3 mL) was stirred at 90° C. for 2 h. The mixture was concentrated in vacuo. The residue taken up in a minimal amount of water, and neutralized with HCl aq. (0.1 mL, 1 N). The precipitate formed was collected by filtration and azeotroped 3 times with methanol to provide the title compound (10 mg, 56% yield) as a white solid. ¹H NMR (400 MHZ, DMSO-d₆) δ ppm 13.26 (s, 1H), 11.74 (s, 1H), 8.34 (s, 1H), 7.13 (dd, J=4.1, 2.6 Hz, 1H), 6.94-6.89 (m, 1H), 6.28 (dd, J=6.0, 2.5 Hz, 1H). LCMS calc. for C₈H₇N₂O₃ [M+H]⁺: m/z=179.0; Found: 179.2.

### INT C57: (Tert-butoxycarbonyl)glycine-2,2-d₂

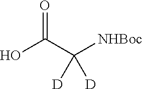 [SMILES: [2H]C([2H])(NC(=O)OC(C)(C)C)C(=O)O]

To a mixture of K₂CO₃ (2.7 g, 20 mmol), glycine-d₂ (700 mg, 9 mmol) in water (20 mL) and DCM (20 mL) was added Boc₂O (3.2 g, 15 mmol), and then stirred at r.t. overnight. The mixture was adjusted to pH˜ 2 with HCl aq. (2.0 M). The organic layers was dried over Na₂SO₄, filtered and concentrated under reduce pressure. The light yellow residue was washed with PE (30 mL) to afford the title compound (800 mg) as a white solid. LCMS calc. for C₇H₁₀D₂NO₄ [M−H]⁻: m/z=176.1; Found: 176.1.

### INT C58: (2-Fluorobenzoyl)glycine-2,2-d₂

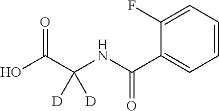 [SMILES: [2H]C([2H])(NC(=O)c1ccccc1F)C(=O)O]

To a mixture of K₂CO₃ (1.38 g, 10 mmol), glycine-d₂ (308 mg, 4 mmol) in water (20 mL) was added 2-fluorobenzoyl chloride (0.8 g, 5 mmol), and then stirred at r.t. overnight. The mixture was adjusted to pH˜2 with HCl aq. (1.0 M). The precipitate was collected by filtration and dried under reduced pressure to afford title compound (420 mg) as a white solid. LCMS calc. for C₉H₅D₂FNO₃ [M−H]⁻: m/z=198.1; Found: 198.1.

### INT C59: 2-(Oxazol-2-ylamino)-2-oxoacetic acid

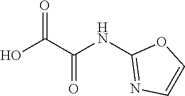 [SMILES: O=C(O)C(=O)Nc1ncco1]

This compound was prepared using procedures analogous to those described for INT C48 step 1-2 using oxazol-2-amine to replace 5-methyl-1H-pyrazol-3-amine in step 1 to afford the title product as a yellow solid. LCMS calc. for C₅H₃N₂O₄ [M−H]⁻: m/z=155.0; Found: 155.1.

### INT C60: 2-Bromo-6-oxo-6,7-dihydrothieno[2,3-b]pyridine-5-carboxylic acid

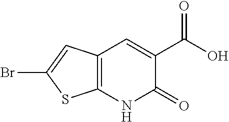 [SMILES: O=C(O)c1cc2cc(Br)sc2[nH]c1=O]

To a solution of methyl 2-bromo-6-oxo-6,7-dihydrothieno[2,3-b]pyridine-5-carboxylate (100 mg, 0.35 mmol) in MeOH/THF/H₂O (5:5:1, 3 mL) was added NaOH (41.6 mg, 1.04 mmol) and the mixture was stirred at r.t. for 4 h. To the reaction mixture was added ice water (10 mL) and the mixture was acidified with HCl aq. (1 N) to pH˜1. The precipitate was collected and washed with water (2 mL), cold diethyl ether (2 mL) and hexane (5 mL), dried to give the title compound (120 mg, crude) as yellow solid. LCMS calc. for C₈H₅BrNO₃S [M+H]⁺: m/z=273.9; Found: 274.0.

### INT C61: 3-Fluoro-1H-pyrrolo[3,2-c]pyridine-2-carboxylic acid

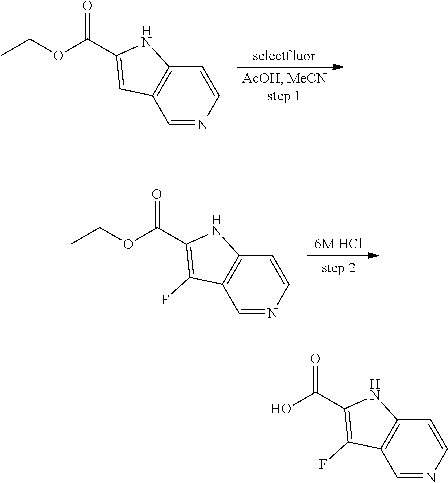

### Step 1: ethyl 3-fluoro-1H-pyrrolo[3,2-c]pyridine-2-carboxylate

To a solution of ethyl 1H-pyrrolo[3,2-c]pyridine-2-carboxylate (200 mg, 1.05 mmol) and selectfluor (1.5 g, 4.21 mmol) in MeCN (5 mL) was added AcOH (7 mg, 0.11 mmol). The mixture was stirred at 70° C. overnight and filtered. The filtrate was concentrated. The residue was purified by Prep-HPLC on a C18 column eluting with MeCN/H₂O (30-50%, with 0.1% NH₄HCO₃) to afford the title compound (20 mg, 9.1% yield) as a white solid. ¹H NMR (400 MHZ, DMSO-d₆) δ ppm 12.14 (s, 1H), 9.00 (d, J=1.1 Hz, 1H), 8.33 (d, J=6.0 Hz, 1H), 7.36 (ddd, J=6.0, 2.4, 1.1 Hz, 1H), 4.38 (q, J=7.1 Hz, 2H), 1.34 (t, J=7.1 Hz, 3H). LCMS calc. for C₁₀H₁₀FN₂O₂ [M+H]⁺: m/z=209.1; Found: 209.1.

### Step 2: 3-fluoro-1H-pyrrolo[3,2-c]pyridine-2-carboxylic acid

A solution of ethyl 3-fluoro-1H-pyrrolo[3,2-c]pyridine-2-carboxylate (20 mg, 0.10 mmol) in HCl aq. (3 mL, 6 M) was stirred at 90° C. overnight. The mixture was concentrated to afford the title compound (17 mg, 98.3% yield) as a white solid which was used in the next step without purification. LCMS calc. for C₈H₆FN₂O₂ [M+H]⁺: m/z=181.0; Found: 181.2.

### INT C62: 4-Hydroxy-5-methylpyrrolo[1,2-b]pyridazine-3-carboxylic acid

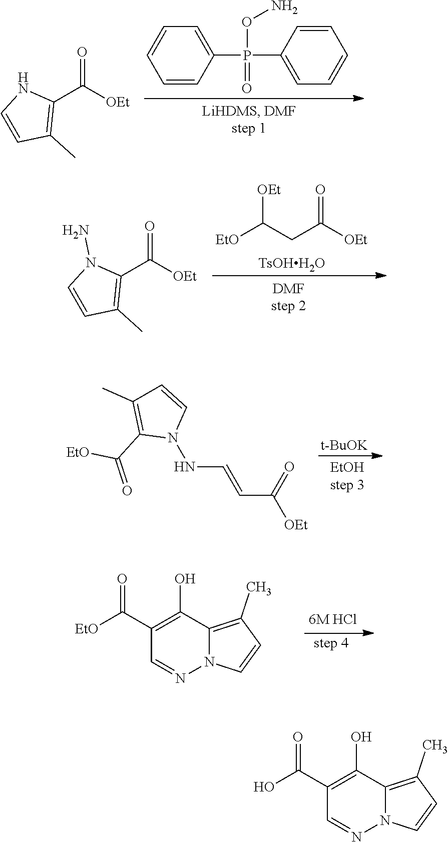

### Step 1: ethyl 1-amino-3-methyl-1H-pyrrole-2-carboxylate

LiHDMS (1.72 g, 9.79 mmol) was added to a solution of ethyl 3-methyl-1H-pyrrole-2-carboxylate (1.5 g, 9.79 mmol) in DMF (80 mL) at 0° C. The mixture was stirred for 10 min., (aminooxy)diphenylphosphine oxide (2.97 g, 12.73 mmol) was added. The mixture was stirred at r.t. overnight, and concentrated. The residue was diluted with water (50 mL) and extracted with EtOAc (50 mL×3). The combined organic layers were washed with sat. aq. NH₄Cl (50 mL×3), dried over Na₂SO₄, filtered and concentrated to afford the title compound (1.47 g, 89.2% yield) as a white solid. ¹H NMR (400 MHZ, DMSO-d₆) δ ppm 6.89 (d, J=2.4 Hz, 1H), 6.15 (s, 2H), 5.82 (dd, J=2.4, 0.6 Hz, 1H), 4.23 (q, J=7.2 Hz, 2H), 2.21 (s, 3H), 1.29 (t, J=7.2 Hz, 3H). LCMS calc. for C₈H₁₃N₂O₂ [M+H]⁺: m/z=169.1; Found: 169.1.

### Step 2: ethyl (E)-1-((3-ethoxy-3-oxoprop-1-en-1-yl)amino)-3-methyl-1H-pyrrole-2-carboxylate

A mixture of 2-((1-amino-3-methyl-1H-pyrrole-2-carbonyl)oxy) ethan-1-ylium (1.24 g, 7.42 mmol), ethyl 3,3-diethoxypropanoate (1.41 g, 7.42 mmol) and TsOH (1.41 g, 7.42 mmol) in DMF (50 mL), was stirred at 90° C. for 2 h. The reaction was quenched with sat. aq. NaHCO₃ and extracted with EtOAc (30 mL×3). The combined organic layers were washed with water and brine, dried over Na₂SO₄, filtered and concentrated. The residue was purified by C18 column eluting with MeOH/H₂O (5-95% with 0.1% NH₄HCO₃) to afford the title compound (1.08 g, 54.68% yield) as a brown oil. ¹H NMR (400 MHZ, DMSO-d₆) δ ppm 9.71 (s, 1H), 7.49 (d, J=13.2 Hz, 1H), 7.04 (d, J=2.8 Hz, 1H), 6.08 (dd, J=2.8, 0.4 Hz, 1H), 4.19 (t, J=7.2 Hz, 2H), 3.98 (dd, J=8.8, 5.4 Hz, 2H), 2.28 (s, 3H), 1.24 (t, J=7.2 Hz, 3H), 1.12 (t, J=7.2 Hz, 3H). LCMS calc. for C₁₃H₁₉N₂O₄ [M+H]⁺: m/z=267.1; Found: 267.2.

### Step 3: ethyl 4-hydroxy-5-methylpyrrolo[1,2-b]pyridazine-3-carboxylate

t-BuOK (455.09 mg, 4.06 mmol) was added to a solution of ethyl (E)-1-((3-ethoxy-3-oxoprop-1-en-1-yl)amino)-3-methyl-1H-pyrrole-2-carboxylate (1.08 g, 4.06 mmol) in EtOH (50 mL). The mixture was stirred at 90° C. for 3 h., quenched with sat. aq. NaHCO₃ and extracted with EtOAc (30 mL×3). The combined organic layers were washed with brine, dried over with Na₂SO₄, filtered and concentrated to afford the title compound (790 mg, 88.45% yield) as a white solid. LCMS calc. for C₁₁H₁₃N₂O₃ [M+H]⁺: m/z=221.1; Found: 221.2.

### Step 4: 4-hydroxy-5-methylpyrrolo[1,2-b]pyridazine-3-carboxylic acid

A mixture of ethyl 4-hydroxy-5-methylpyrrolo[1,2-b]pyridazine-3-carboxylate (100 mg, 0.05 mmol) and NaOH (0.1 mL, 8 M) in EtOH (1 mL) was stirred at 80° C. for 2 h. The reaction mixture was cooled to r.t., diluted with EtOAc (20 mL) and extracted with water (5 mL×3). The combined aqueous layers were adjusted to pH to 2-3 with HCl aq. (6 M). The precipitate formed was collected by filtration and dried in vacuo to afford the title product (30 mg, 34.38% yield). ¹H NMR (400 MHZ, DMSO-d₆) δ ppm 8.13 (s, 1H), 7.74 (d, J=2.4 Hz, 1H), 6.58 (d, J=2.4 Hz, 1H), 2.50 (s, 3H). LCMS calc. for C₉H₉N₂O₃ [M+H]⁺: m/z=193.1; Found: 193.1.

### INT C63: 6-Hydroxythieno[3,2-b]pyridine-5-carboxylic acid

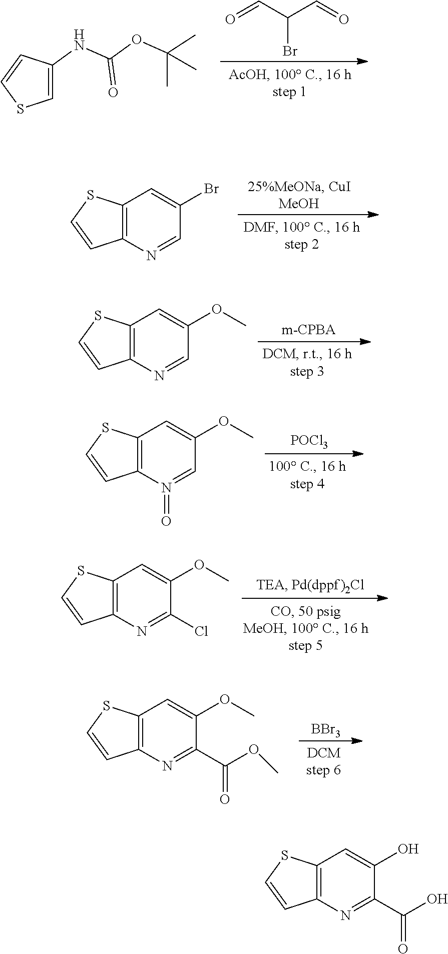

### Step 1: 6-bromothieno[3,2-b]pyridine

A mixture of tert-butyl thiophen-3-ylcarbamate (3 g, 15 mmol) and 2-bromomalonaldehyde (2.26 g, 15 mmol) in acetic acid (40 mL) was stirred at 100° C. overnight. The reaction mixture was concentrated under reduced pressure, diluted with H₂O (30 mL) and extracted with DCM (50 mL×3). The combined organic layers were washed with sat. aq. NH₄Cl and brine, dried over Na₂SO₄, filtered and concentrated. The residue was purified by flash chromatography on a silica gel column eluting with EtOAc/PE (10%) to afford the title product (1.93 g, 60.1% yield) as white solid. LCMS calc. for C₇H₅BrNS [M+H]⁺: m/z=213.9; Found: 213.9/215.9.

### Step 2: 6-methoxythieno[3,2-b]pyridine

A mixture of 6-bromothieno[3,2-b]pyridine (1.93 g, 9 mmol), CuI (1.71 g, 9 mmol) and MeONa (25%) (4.86 g, 90 mmol) in DMF (20 mL) was stirred at 100° C. under N₂ overnight. The reaction mixture was quenched with H₂O (30 mL) and extracted with DCM (50 mL×3). The combined organic layers were washed with sat. aq. NH₄Cl and brine, dried over Na₂SO₄, filtered and concentrated. The residue was purified by flash chromatography on a silica gel column eluting with EtOAc/PE (10%) to afford the title product (1.30 g, 84% yield) as a yellow solid. LCMS calc. for C₈H₈NOS [M+H]⁺: m/z=166.0; Found: 166.0.

### Step 3: 6-methoxythieno[3,2-b]pyridine 4-oxide

To a solution of 6-methoxythieno[3,2-b]pyridine (1.3 g, 7.87 mmol) in DCM (20 mL) was added m-CPBA (1.35 g, 7.87 mmol). The mixture was stirred at r.t. overnight. The reaction mixture was quenched with H₂O (30 mL) and extracted with DCM (50 mL×3). The combined organic layers were washed with sat. aq. NaHCO₃ and brine, dried over Na₂SO₄, filtered and concentrated. The residue was purified by flash chromatography on a silica gel column eluting with EtOAc/PE (17%) to afford the title product (1.23 g, 86% yield) as a white solid. LCMS calc. for C₈H₈NO₂S [M+H]⁺: m/z=182.0; Found: 182.0.

### Step 4: 5-chloro-6-methoxythieno[3,2-b]pyridine

A mixture of 6-methoxythieno[3,2-b]pyridine 4-oxide (198 mg, 0.88 mmol) in POCl₃ (2 mL) was stirred at 100° C. overnight. The reaction mixture was concentrated, diluted with EtOAc (50 mL), washed with sat. aq. NaHCO₃, sat. aq. NH₄Cl and brine, dried over Na₂SO₄, filtered and concentrated under reduced pressure. The residue was purified by flash chromatography on a silica gel column eluting with EtOAc/PE (15%) to afford the title product (160 mg, 74% yield) as a yellow solid. LCMS calc. for C₈H₇ClNOS [M+H]⁺: m/z=200.0; Found: 200.1.

### Step 5. methyl 6-methoxythieno[3,2-b]pyridine-5-carboxylate

To a solution of 5-chloro-6-methoxythieno[3,2-b]pyridine (500 mg, 2.51 mmol) in MeOH (8 mL) was added Pd(dppf)₂Cl₂ (203.5 mg, 0.25 mmol) and TEA (507 mg, 5.02 mmol). The mixture was stirred at r.t. under 50 psi of CO pressure for 16 h. The mixture was concentrated, diluted with H₂O (25 mL) and extracted with DCM (50 mL×3). The organic layers were washed with sat. aq. NH₄Cl and brine, dried over Na₂SO₄, filtered and concentrated. The residue was purified by flash chromatography on a silica gel column eluting with EtOAc/PE (0-20%) to afford the title product (280 mg, 50% yield) as a white solid. LCMS calc. for C₁₀H₁₀NO₃S [M+H]⁺: m/z=224.0; Found: 224.1.

### Step 6:6-hydroxythieno[3,2-b]pyridine-5-carboxylic acid

To a solution of methyl 6-methoxythieno[3,2-b]pyridine-5-carboxylate (260 mg, 1.43 mmol) in DCM (2 mL) was added BBr₃ (1.41 g, 5.72 mmol)) at 0° C. The reaction mixture was allowed to warm to r.t. and stirred for 16 h. The solvent was removed, and ice water (15 mL) was added. The precipitate was collected, washed with water (2 mL), cold diethyl ether (2 mL) and hexane (5 mL), and dried in vacuo to afford the title product (110 mg, 45.2% yield) as a yellow solid. LCMS calc. for C₈H₆NO₃S [M+H]⁺: m/z=196.0; Found: 196.2.

### INT C64: 2-Chloro-7-hydroxythiazolo[5,4-b]pyridine-6-carboxylic acid

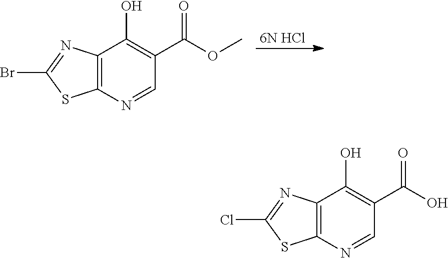

A mixture of methyl 2-bromo-7-hydroxythiazolo[5,4-b]pyridine-6-carboxylate (80 mg, 0.28 mmol) in 6 N HCl (5 mL) was heated at 90° C. for 45 mins. The reaction mixture was concentrated to afford the title product as HCl salt (80 mg, crude) which was used directly in the next step directly. LCMS calc. for C₇H₄ClN₂O₃S [M+H]⁺: m/z=231.0; Found: 231.1.

### INT C65: 5-Methoxy-3-oxo-2,3-dihydropyridazine-4-carboxylic acid

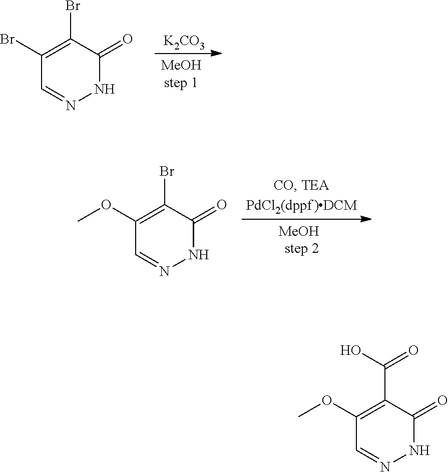

### Step 1: 4-bromo-5-methoxypyridazin-3 (2H)-one

A mixture of 4,5-dibromopyridazin-3 (2H)-one (10.0 g, 39.4 mmol) and K₂CO₃ (10.9 g, 78.8 mmol) in MeOH (100 mL) was stirred at 80° C. for 3 days. The solvent was removed under reduced pressure. The residue was diluted with water, and acetic acid was added until pH˜4. The precipitate formed was collected by filtration, washed with water, and dried to afford the title compound (7.5 g) without further purification. LCMS calc. for C₅H₆BrN₂O₂ [M+H]: m/z=205.0; Found: 205.2.

### Step 2: 5-methoxy-3-oxo-2,3-dihydropyridazine-4-carboxylic acid

A mixture of 4-bromo-5-methoxypyridazin-3 (2H)-one (2.0 g, 9.75 mmol), TEA (9.9 g, 97.5 mmol) and PdCl₂ (dppf)·DCM (796 mg, 0.98 mmol) in MeOH (30 mL) was stirred in an autoclave at 120° C. for 60 h. under CO. The mixture was concentrated under reduced pressure. The residue was poured into water, extracted with EtOAc (100 mL×3). The combined organic layers were washed with brine (30 mL), dried over Na₂SO₄, filtered, and concentrated under reduced pressure. The residue was purified by Prep-HPLC on a C18 column eluting with MeCN/H₂O (2-8%, with 0.1% FA) to afford the title compound (1.1 g, 66.3% yield) as a white solid. LCMS calc. for C₆H₇N₂O₄ [M+H]⁺: m/z=171.0; Found: 171.2.

### INT C66: 2-Oxo-5-(trifluoromethyl)-2,3-dihydrooxazole-4-carboxylic acid

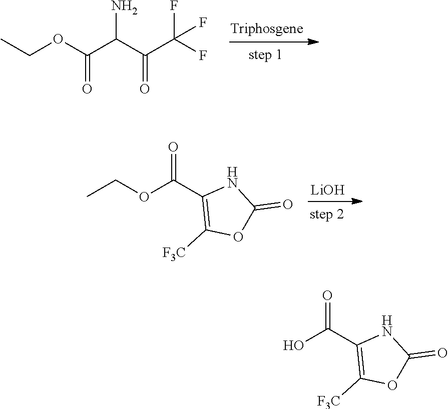

### Step 1: ethyl 2-oxo-5-(trifluoromethyl)-2,3-dihydrooxazole-4-carboxylate

To a stirred solution of ethyl 2-amino-4,4,4-trifluoro-3-oxobutanoate (1.5 g, 7.54 mmol) in THF (10 mL) was added TEA (2.28 g, 22.61 mmol) at −50° C. under N₂ atmosphere and stirred for 30 min. Triphosgene (896 mg, 3.02 mmol) in THF (5 mL) was added at −50° C. The resulting mixture was stirred for additional 3 h. at 0° C., diluted with aq. NH₄Cl, and extracted with DCM (10 mL×3). The combined organic layers were dried over Na₂SO₄, filtered and concentrated. The residue was purified by flash chromatography on a silica gel column eluting with EtOAc/PE (0-35%) to afford the target compound (650 mg, crude) as light green oil. LCMS calc. for C₇H₇F₃NO₄ [M+H]⁺: m/z=226.0; Found: 226.1.

### Step 2: 2-oxo-5-(trifluoromethyl)-2,3-dihydrooxazole-4-carboxylic acid

A mixture of ethyl 2-oxo-5-(trifluoromethyl)-2,3-dihydrooxazole-4-carboxylate (300 mg, 1.33 mmol) and LiOH (224 mg, 5.33 mmol) in THF (3 mL) and H₂O (3 mL) was stirred at r.t. for 3 h. The reaction mixture was adjusted to pH to 4 with HCl aq. (1 N), concentrated in vacuo. The residue was purified by Prep-HPLC on a C18 column eluting with MeCN/H₂O (30-50%, with 0.1% FA) to afford the title compound (50 mg, 19% yield) as a green solid. LCMS calc. for C₅H₃F₃NO₄ [M+H]⁺: m/z=198.0; Found: 198.1.

### INT C67: 2-((3-Methylisothiazol-5-yl)amino)-2-oxoacetic acid

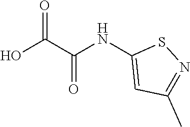

This compound was prepared using procedures analogous to those described for INT C48 step 1-2 using 3-methylisothiazol-5-amine hydrogen chloride salt to replace 5-methyl-1H-pyrazol-3-amine in step 1 to afford the title product as a white solid. LCMS calc. for C₆H₅N₂O₃S [M−H]⁻: m/z=185.0; Found: 185.0.

### INT C68: 2-oxo-2-(Pyrimidin-4-ylamino)acetic acid

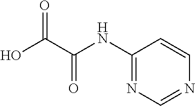 [SMILES: O=C(O)C(=O)Nc1ccncn1]

This compound was prepared using procedures analogous to those described for INT C48 step 1-2 using pyrimidin-4-amine to replace 5-methyl-1H-pyrazol-3-amine in step 1 to afford the title product as a brown solid. LCMS calc. for C₆H₄N₃O₃ [M−H]⁻: m/z=166.0; Found: 166.1.

### INT C69: 2-(Isoxazol-5-ylamino)-2-oxoacetic acid

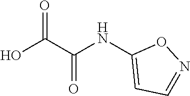 [SMILES: O=C(O)C(=O)Nc1ccno1]

This compound was prepared using procedures analogous to those described for INT C48 step 1-2 using isoxazol-5-amine to replace 5-methyl-1H-pyrazol-3-amine in step 1 to afford the title product as a white solid. LCMS calc. for C₅H₃N₂O₄ [M−H]⁻: m/z=155.0; Found: 155.1.

### INT C70: 2-((3-Methylisoxazol-5-yl)amino)-2-oxoacetic acid

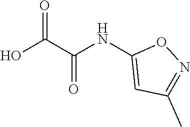

### Step 1: ethyl 2-((3-methylisoxazol-5-yl)amino)-2-oxoacetate

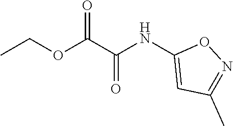

To a mixture of 3-methylisoxazol-5-amine (500 mg, 5.10 mmol) in DCM (5 mL) was added TEA (1.55 g, 15.29 mmol) and added dropwise ethyl 2-chloro-2-oxoacetate (696 mg, 5.10 mmol) at 0° C. The reaction mixture was stirred at r.t. for overnight. The reaction mixture was quenched with H₂O (30 mL), and then extracted with DCM (3×30 mL). The combined organic layer was washed with brine (30 mL), dried over Na₂SO₄, and concentrated under reduced pressure to afford the title compound (500 mg, 50% yield) as a light brown solid. LCMS calc. for C₈H₁₁N₂O₄ [M+H]⁺: m/z=199.1; Found: 199.1.

### Step 2: 2-((3-methylisoxazol-5-yl)amino)-2-oxoacetic acid

To a mixture of ethyl 2-((3-methylisoxazol-5-yl)amino)-2-oxoacetate (500 mg, 2.52 mmol) in THF (5 mL) was added LiOH·H₂O (212 mg, 5.05 mmol), H₂O (5 mL). The reaction mixture was stirred at r.t. for 2 h. The mixture was adjusted pH˜2-3 and then concentrated under reduced pressure. The residue was purified by prep-HPLC on a C18 column eluting with CH₃CN/H₂O (2%) to afford the title compound (300 mg, 46% yield) as a white solid. LCMS calc. for C₆H₅N₂O₄ [M−H]⁻: m/z=169.0; Found: 169.1.

### INT C71: 2-((4-Methylthiazol-2-yl)amino)-2-oxoacetic acid

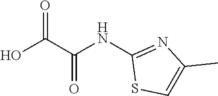

### Step 1: ethyl 2-((4-methylthiazol-2-yl)amino)-2-oxoacetate

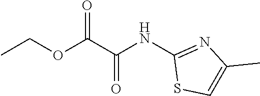

To a mixture of 4-methylthiazol-2-amine (1.14 g, 10 mmol) in DCM (20 mL) was added TEA (1.6 g, 15 mmol) and added dropwise ethyl 2-chloro-2-oxoacetate (1.63 g, 12 mmol) at 0° C. The mixture was stirred at r.t. overnight. The reaction mixture was quenched with H₂O (30 mL), and then extracted with DCM (30 mL×3). The combined organic layers were washed with brine (30 mL), dried over Na₂SO₄, filtered and concentrated under reduced pressure. The residue was purified by flash chromatography on a C18 column eluting with MeCN/H₂O (10-30%) to afford the title compound (1.2 g) as a yellow solid. LCMS calc. for C₈H₁₁N₂O₃S [M+H]⁺: m/z=215.0; Found: 215.1.

### Step 2: 2-((4-methylthiazol-2-yl)amino)-2-oxoacetic acid

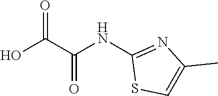

To a mixture of ethyl 2-((4-methylthiazol-2-yl)amino)-2-oxoacetate (1.1 g, 5 mmol) in THF (10 mL) was added LiOH·H₂O (315 mg, 7.5 mmol) in H₂O (10 mL). The mixture was stirred at r.t. for 1 h. The reaction mixture was adjusted to pH˜2-3 with HCl aq. (2 M) and then concentrated under reduced pressure. The residue was purified by flash chromatography on a C18 column eluting with MeCN/H₂O (0-10%) to afford the title compound (0.4 g) as a white solid. LCMS calc. for C₆H₇N₂O₃S [M+H]⁺: m/z=187.0; Found: 187.0.

### INT C72: 2-((5-Chloro-1,3,4-thiadiazol-2-yl)amino)-2-oxoacetic acid

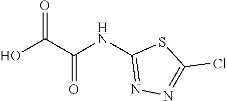 [SMILES: O=C(O)C(=O)Nc1nnc(Cl)s1]

### Step 1: ethyl 2-((5-chloro-1,3,4-thiadiazol-2-yl)amino)-2-oxoacetate

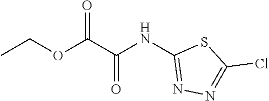

To a mixture of 5-chloro-1,3,4-thiadiazol-2-amine (300 mg, 2.21 mmol), TEA (448 mg, 4.43 mmol) in DCM (20 mL) was added dropwise ethyl 2-chloro-2-oxoacetate (302 mg, 2.21 mmol) at 0° C. The mixture was stirred at r.t. overnight. The reaction mixture was quenched with H₂O (30 mL), and then extracted with DCM (30 mL×3). The combined organic layers were washed with brine (30 mL), dried over Na₂SO₄, filtered and concentrated under reduced pressure. The residue was purified by flash chromatography on a C18 column eluting with MeCN/H₂O (0-20%) to afford the title compound (300 mg) as a yellow solid. LCMS calc. for C₆H₅ClN₃O₃S [M−H]⁻: m/z=234.0; Found: 234.0.

### Step 2: 2-((5-chloro-1,3,4-thiadiazol-2-yl)amino)-2-oxoacetic acid

To a solution of ethyl 2-((5-chloro-1,3,4-thiadiazol-2-yl)amino)-2-oxoacetate (300 mg, 1.27 mmol) in THF (3 mL) was added LiOH·H₂O (107 mg, 2.55 mmol) in H₂O (3 mL). The mixture was stirred at r.t. for 2 h. The reaction mixture was adjusted to pH˜2-3 with HCl aq. (1 N) and then concentrated under reduced pressure. The residue was purified by flash chromatography on a C18 column eluting with MeCN/H₂O (0-15%) to afford the title compound (100 mg) as a white solid. LCMS calc. for C₄H₁ClN₃O₃S [M−H]⁻: m/z=206.0; Found: 206.0.

### INT C73: 2-((4-Methyloxazol-2-yl)amino)-2-oxoacetic acid

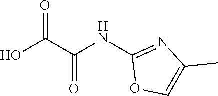

### Step 1: ethyl 2-((4-methyloxazol-2-yl)amino)-2-oxoacetate

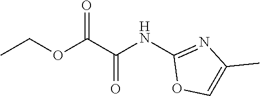

To a mixture of 4-methyloxazol-2-amine (1.0 g, 10 mmol), TEA (1.6 g, 15 mmol) in DCM (20 mL) was added dropwise ethyl 2-chloro-2-oxoacetate (1.63 g, 12 mmol) at 0° C. The mixture was stirred at r.t. overnight. The reaction mixture was quenched with H₂O (30 mL), and then extracted with DCM (30 mL×3). The combined organic layers were washed with brine (30 mL), dried over Na₂SO₄, filtered and concentrated under reduced pressure. The residue was purified by prep-HPLC on a C18 column eluting with MeCN/H₂O (15-30%) to afford the title compound (0.8 g) as a brown solid. LCMS calc. for C₈H₁₁N₂O₄ [M+H]⁺: m/z=199.1; Found: 199.1.

### Step 2: 2-((4-methyloxazol-2-yl)amino)-2-oxoacetic acid

To a mixture of ethyl 2-((4-methyloxazol-2-yl)amino)-2-oxoacetate (0.8 g, 4 mmol) in THF/water (v/v=2:1, 5 mL) was added NaOH (240 mg, 6 mmol) at 0° C. The mixture was stirred at r.t. for 3 h., adjusted to pH 6˜7 with HCl aq. (2 M), and then concentrated under reduced pressure to afford the title compound (680 mg) as a white solid. LCMS calc. for C₆H₅N₂O₄ [M−H]⁻: m/z=169.0; Found: 169.0.

### INT C74: 2-Morpholino-2-oxoacetic acid

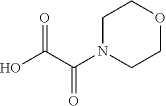 [SMILES: O=C(O)C(=O)N1CCOCC1]

### Step 1: ethyl 2-morpholino-2-oxoacetate

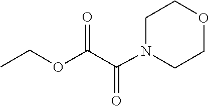

To a mixture of morpholine (1.00 g, 11.48 mmol) and TEA (3.48 g, 34.44 mmol) in DCM (20 mL) was added dropwise ethyl 2-chloro-2-oxoacetate (1.57 mg, 11.48 mmol) at 0° C. The mixture was stirred at r.t. overnight. The reaction mixture was quenched with H₂O (30 mL), and then extracted with DCM (30 mL×3). The combined organic layers were washed with brine (30 mL), dried over Na₂SO₄, filtered and concentrated under reduced pressure. The residue was purified by flash chromatography on a C18 column eluting with MeCN/H₂O (10-30%) to afford the title compound (500 mg) as a light yellow solid. LCMS calc. for C₈H₁₄NO₄ [M+H]⁺: m/z=188.1; Found: 188.1.

### Step 2: 2-morpholino-2-oxoacetic acid

To a solution of ethyl 2-morpholino-2-oxoacetate (500 mg, 2.67 mmol) in THF (5 mL) was added LiOH·H₂O (224 mg, 5.34 mmol) in H₂O (5 mL). The mixture was stirred at r.t. for 2 h. The mixture was adjusted to pH˜2-3 with HCl aq. (1 N) and then concentrated under reduced pressure. The residue was purified by flash chromatography on a C18 column eluting with MeCN/H₂O (0-2%) to afford the title compound (100 mg) as a white solid. LCMS calc. for C₆H₈NO₄ [M−H]⁻: m/z=158.1; Found: 158.1.

### INT C75: (3-Fluoro-6-(methylamino)picolinoyl)glycine

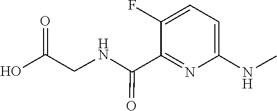 [SMILES: CNc1ccc(F)c(C(=O)NCC(=O)O)n1]

### Step 1: 3-fluoro-6-(methylamino)picolinic acid

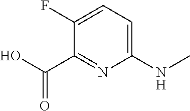

A solution of 3,6-difluoropicolinic acid (160 mg, 1 mmol) in MeNH₂ solution (2 mL, 20% in MeOH) was stirred at 100° C. overnight in a sealed tube. The reaction mixture was concentrated under reduced pressure to afford the title compound (170 mg) as a white solid. LCMS calc. for C₇H₈FN₂O₂ [M+H]⁺: m/z=171.1; Found: 171.1.

### Step 2: tert-butyl (3-fluoro-6-(methylamino)picolinoyl)glycinate

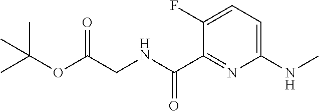 [SMILES: CNc1ccc(F)c(C(=O)NCC(=O)OC(C)(C)C)n1]

A mixture of 3-fluoro-6-(methylamino)picolinic acid (170 mg, 1 mmol), tert-butyl glycinate (260 mg, 1 mmol), EDCI (390 mg, 2 mmol), HOBt (270 mg, 2 mmol) and TEA (300 mg, 3 mmol) in DMF (4 mL) was stirred at r.t. overnight. The mixture was concentrated under reduced pressure. The residue was purified by flash chromatography on a C18 column eluting with MeCN/H₂O (45-65% with 0.1% TFA) to afford the title compound (230 mg) as a colorless oil. LCMS calc. for C₁₃H₁₉FN₃O₃ [M+H]⁺: m/z=284.1; Found: 284.1.

### Step 3: (3-fluoro-6-(methylamino)picolinoyl)glycine

A solution of tert-butyl (3-fluoro-6-(methylamino)picolinoyl)glycinate (230 mg, 0.81 mmol) in 4.0 M HCl/1,4-dioxane (2 mL) was stirred at r.t. overnight. The reaction mixture was concentrated under reduced pressure to afford the title compound (140 mg) as a white solid. LCMS calc. for C₉H₁₁FN₃O₃ [M+H]⁺: m/z=228.1; Found: 228.1.

### INT C76: (7H-Pyrrolo[2,3-d]pyrimidin-4-yl)glycine

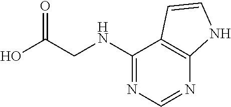 [SMILES: O=C(O)CNc1ncnc2[nH]ccc12]

### Step 1: tert-butyl (7H-pyrrolo[2,3-d]pyrimidin-4-yl)glycinate

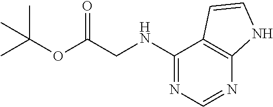 [SMILES: CC(C)(C)OC(=O)CNc1ncnc2[nH]ccc12]

To a solution of 4-bromo-7H-pyrrolo[2,3-d]pyrimidine (500 mg, 2.5 mmol) in MeCN (10 mL) were added tert-butyl glycinate (3.28 g, 25 mmol) and TEA (753 mg, 7.5 mmol) at 0° C. The resulting mixture was stirred at 90° C. for 48 h. The reaction solution was quenched with H₂O (100 mL) and extracted with EtOAc (100 mL×3). The combined organic layers were washed with brine, dried over anhydrous Na₂SO₄, filtered and concentrated under reduced pressure to afford the crude title compound (400 mg) as a white solid. LCMS calc. for C₁₂H₁₅N₄O₂ [M−H]⁻: m/z=247.1; Found: 247.1.

### Step 2: (7H-pyrrolo[2,3-d]pyrimidin-4-yl)glycine

A solution of crude tert-butyl (7H-pyrrolo[2,3-d]pyrimidin-4-yl)glycinate (400 mg, 1.6 mmol) in DCM/TFA (v:v=3:1, 10 mL) was stirred at r.t. for 1 h. The reaction mixture was concentrated under reduced pressure to afford the crude title compound (280 mg) as an off-white solid. LCMS calc. for C₈H₇N₄O₂ [M−H]⁻: m/z=191.1; Found: 191.0.

### INT C77: (5-Hydroxypyrimidine-4-carbonyl)glycine

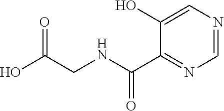 [SMILES: O=C(O)CNC(=O)c1ncncc1O]

### Step 1: tert-butyl (5-methoxypyrimidine-4-carbonyl)glycinate

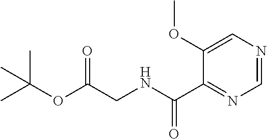 [SMILES: COc1cncnc1C(=O)NCC(=O)OC(C)(C)C]

A mixture of 5-methoxypyrimidine-4-carboxylic acid (300 mg, 2 mmol), tert-butyl glycinate (520 mg, 2 mmol), EDCI (570 mg, 3 mmol), HOBt (400 mg, 3 mmol) and TEA (300 mg, 3 mmol) in DMF (5 mL) was stirred at 40° C. overnight. The mixture was concentrated under reduced pressure. The residue was purified by flash chromatography on a C18 column eluting with MeCN/H₂O (60-65% with 0.1% TFA) to afford the title compound (330 mg) as a colorless oil. LCMS calc. for C₁₂H₁₈N₃O₄ [M+H]⁺: m/z=268.1; Found: 268.1.

### Step 2: (5-hydroxypyrimidine-4-carbonyl)glycine

To a solution of tert-butyl (5-methoxypyrimidine-4-carbonyl)glycinate (330 mg, 1.23 mmol) in NMP (10 mL) was added NaSEt (320 mg, 0.4 mmol) and stirred at 100° C. for 1 h. The reaction mixture was concentrated under reduced pressure. The residue was purified by flash chromatography on a C18 column eluting with MeCN/H₂O (60-65% with 0.5% HCl) to afford the title compound (130 mg) as a white solid. LCMS calc. for C₇H₆N₃O₄ [M−H]⁻: m/z=196.0; Found: 196.0.

### INT C78: (3-Fluoro-6-oxo-1,6-dihydropyridine-2-carbonyl)glycine

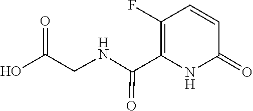 [SMILES: O=C(O)CNC(=O)c1[nH]c(=O)ccc1F]

### Step 1: tert-butyl (3-fluoro-6-oxo-1,6-dihydropyridine-2-carbonyl)glycinate

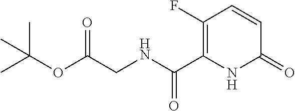 [SMILES: CC(C)(C)OC(=O)CNC(=O)c1[nH]c(=O)ccc1F]

A mixture of 3-fluoro-6-oxo-1,6-dihydropyridine-2-carboxylic acid (150 mg, 1.0 mmol), tert-butyl glycinate (260 mg, 2 mmol), EDCI (390 mg, 2 mmol), HOBt (270 mg, 2 mmol) and TEA (300 mg, 3 mmol) in DMF (4 mL) was stirred at r.t. overnight. The mixture was concentrated under reduced pressure. The residue was purified by flash chromatography on a C18 column eluting with MeCN/H₂O (65-75% with 0.1% TFA) to afford the title compound (180 mg) as a colorless oil. LCMS calc. for C₁₂H₁₆FN₂O₄ [M+H]⁺: m/z=271.1; Found: 271.1.

### Step 2: (3-fluoro-6-oxo-1,6-dihydropyridine-2-carbonyl)glycine

A solution of tert-butyl (3-fluoro-6-oxo-1,6-dihydropyridine-2-carbonyl)glycinate (54 mg, 0.2 mmol) in 4.0 M HCl/1,4-dioxane (2 mL) was stirred at r.t. overnight. The reaction mixture was concentrated under reduced pressure to afford the title compound (40 mg) as a white solid. LCMS calc. for C₈H₆FN₂O₄ [M−H]⁻: m/z=213.0; Found: 213.0.

### INT C79: (6-Amino-3-fluoropicolinoyl)glycine

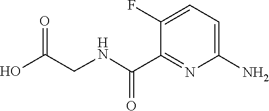 [SMILES: Nc1ccc(F)c(C(=O)NCC(=O)O)n1]

### Step 1: 6-amino-3-fluoropicolinic acid

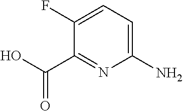 [SMILES: Nc1ccc(F)c(C(=O)O)n1]

A solution of 3,6-difluoropicolinic acid (80 mg, 0.5 mmol) in NH₃ solution (2 mL, 7.0 M in MeOH) was stirred at 100° C. for 3 days in a sealed tube. The reaction mixture was concentrated under reduced pressure to afford the title compound (75 mg) as a white solid. LCMS calc. for C₆H₆FN₂O₂ [M+H]⁺: m/z=157.0; Found: 157.0.

### Step 2: tert-butyl (6-amino-3-fluoropicolinoyl)glycinate

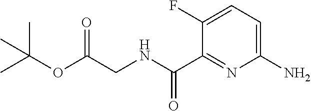 [SMILES: CC(C)(C)OC(=O)CNC(=O)c1nc(N)ccc1F]

A mixture of 3-fluoro-6-hydroxypicolinic acid (75 mg, 0.5 mmol), tert-butyl glycinate (130 mg, 1 mmol), EDCI (193 mg, 1 mmol), HOBt (135 mg, 1 mmol) and TEA (150 mg, 1.5 mmol) in DMF (2 mL) was stirred at r.t. overnight. The mixture was concentrated under reduced pressure. The residue was purified by flash chromatography on a C18 column eluting with MeCN/H₂O (45-65% with 0.1% TFA) to afford the title compound (48 mg) as a colorless oil. LCMS calc. for C₁₂H₁₇FN₃O₃ [M+H]⁺: m/z=270.1; Found: 270.1.

### Step 3: (6-amino-3-fluoropicolinoyl)glycine

A solution of tert-butyl (6-amino-3-fluoropicolinoyl)glycinate (48 mg, 0.18 mmol) in 4.0 M HCl/1,4-dioxane (2 mL) was stirred at r.t. overnight. The reaction mixture was concentrated under reduced pressure to afford the title compound (30 mg) as a white solid. LCMS calc. for C₈H₉FN₃O₃ [M+H]⁺: m/z=214.1; Found: 214.0.

### INT C80: (5-Hydroxy-2-methylpyrimidine-4-carbonyl)glycine

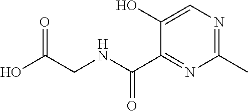

### Step 1: 5-(benzyloxy)-2-methylpyrimidine-4-carboxylic acid

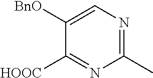 [SMILES: Cc1ncc(OCc2ccccc2)c(C(=O)O)n1]

A mixture of 5-bromo-2-methylpyrimidine-4-carboxylic acid (216 mg, 1 mmol), CuBr (143 mg, 1 mmol), Cs₂CO₃ (650 mg, 2 mmol) and BnOH (216 mg, 2 mmol) in DMF (5 mL) was stirred at 80° C. overnight. The reaction mixture was diluted with EtOAc (60 mL), washed with water (20 mL×2), dried over anhydrous Na₂SO₄, filtered and concentrated under reduced pressure. The residue was purified by flash chromatography on a C18 column eluting with MeCN/H₂O (60-65% with 0.1% TFA) to afford the title compound (50 mg) as a yellow solid. LCMS calc. for C₁₃H₁₁N₂O₃ [M−H]⁻: m/z=243.1; Found: 243.1.

### Step 2: tert-butyl (5-(benzyloxy)-2-methylpyrimidine-4-carbonyl)glycinate

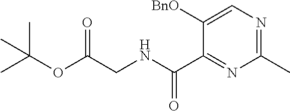 [SMILES: Cc1ncc(OCc2ccccc2)c(C(=O)NCC(=O)OC(C)(C)C)n1]

A mixture of 5-(benzyloxy)-2-methylpyrimidine-4-carboxylic acid (50 mg, 0.2 mmol), tert-butyl glycinate (110 mg, 0.4 mmol), EDCI (76 mg, 0.4 mmol), HOBt (54 mg, 0.4 mmol) and TEA (101 mg, 1 mmol) in DMF (2 mL) was stirred at 40° C. overnight. The reaction mixture was concentrated under reduced pressure. The residue was purified by flash chromatography on a C18 column eluting with MeCN/H₂O (60-75% with 0.1% TFA) to afford the title compound (60 mg) as a colorless oil. LCMS calc. for C₁₉H₂₄N₃O₄ [M+H]⁺: m/z=358.2; Found: 358.2.

### Step 3: (5-hydroxy-2-methylpyrimidine-4-carbonyl)glycine

A solution of tert-butyl (5-methoxypyrimidine-4-carbonyl)glycinate (60 mg, 0.18 mmol) in 5.0 M HBr/1,4-dioxane (1 mL) was stirred at r.t. overnight. The reaction mixture was concentrated under reduced pressure to afford the title compound (35 mg) as a yellow solid. LCMS calc. for C₈H₈N₃O₄ [M−H]⁻: m/z=210.1; Found: 210.1.

### INT C81: 5-Hydroxy-6-methylpyrimidine-4-carboxylic acid

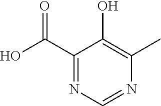

### Step 1: 4-chloro-5-methoxy-6-methylpyrimidine

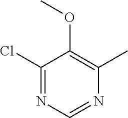

4,6-dichloro-5-methoxypyrimidine (10.0 g, 55.87 mmol) was dissolved in THF (800 mL) under N₂ and cooled to 0° C., MeMgCl (20 mL, 3.0 M, 60.00 mmol) was added dropwise. The reaction mixture was stirred at 0° C. for 2 h. The mixture was quenched with HCl aq. (1 N), and extracted with MTBE (60 mL×2). The combined organic layers were washed with brine, dried over Na₂SO₄, filtered and concentrated under reduced pressure. The residue was purified by flash chromatography on a silica gel column eluting with EA/PE (10-20%) to afford the title compound (8.0 g, 90% yield) as a white solid. ¹H NMR (400 MHZ, CDCl₃) δ ppm 8.61 (s, 1H), 3.91 (s, 3H), 2.56 (s, 3H). LCMS calc. for C₆H₈ClN₂O [M+H]⁺: m/z=159.0; Found: 159.0.

### Step 2: methyl 5-methoxy-6-methylpyrimidine-4-carboxylate

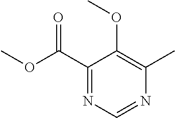

To a solution of 4-chloro-5-methoxy-6-methylpyrimidine (25.0 g, 158 mmol) in MeOH (126 mL) were added Pd(dppf)Cl₂·DCM (6.2 g, 7.57 mmol) and Et₃N (44 mL, 316 mmol). The mixture was stirred at 100° C. overnight under CO atmosphere (40 bar). After cooling to r.t., the solid were collected by filtration and washed with MeOH. The filtrate was concentrated under reduced pressure. The residue was diluted with water, and extracted with EtOAc (100 mL×3). The combined organic layers were concentrated under reduced pressure. The residue was purified by flash chromatography on a silica gel column eluting with EA/PE (5-30%) to afford the title compound (12.2 g, 43.4% yield) as an oil. ¹H NMR (400 MHZ, CDCl₃) δ ppm 8.89 (s, 1H), 4.02 (s, 3H), 3.92 (s, 3H), 2.59 (s, 3H). LCMS calc. for C₈H₁₁N₂O₃ [M+H]⁺: m/z=183.1; Found: 183.1.

### Step 3: 5-hydroxy-6-methylpyrimidine-4-carboxylic acid

Methyl 5-methoxy-6-methylpyrimidine-4-carboxylate (3.0 g, 16.47 mmol) was dissolved in HBr (40% in water) (20.0 mL, 98.8 mmol). The mixture was heated at 40° C. for 12 h., and treated with HI (57% in water) (22.0 mL, 98.8 mmol). The mixture was stirred for additional 6 h., adjusted to pH 3˜4 with NaOH (50% in water) at 0° C. The solid formed was collected by filtration, and dried under vacuum to afford the title compound (2.4 g, 94.6% yield) as a white solid. ¹H NMR (400 MHZ, DMSO-d₆) δ ppm 8.35 (d, J=4.6, 1H), 2.36 (s, 3H).

### INT D1: 2-(2-(3,6-Dihydro-2H-pyran-4-yl)-5-ethyl-6-(4-((2-fluorobenzoyl)glycyl)piperazin-1-yl)-7-oxo-[1,2,4]triazolo[1,5-a]pyrimidin-4(7H)-yl)acetic acid

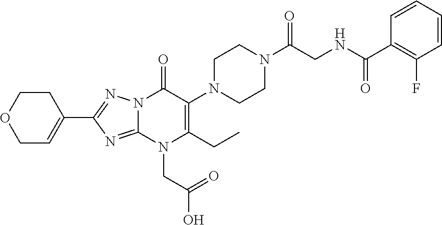 [SMILES: CCc1c(N2CCN(C(=O)CNC(=O)c3ccccc3F)CC2)c(=O)n2nc(C3=CCOCC3)nc2n1CC(=O)O]

### Step 1: tert-butyl 4-(2-(3,6-dihydro-2H-pyran-4-yl)-5-ethyl-4-(2-methoxy-2-oxoethyl)-7-oxo-4,7-dihydro-[1,2,4]triazolo[1,5-a]pyrimidin-6-yl)piperazine-1-carboxylate

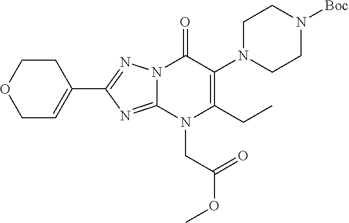 [SMILES: CCc1c(N2CCN(C(=O)OC(C)(C)C)CC2)c(=O)n2nc(C3=CCOCC3)nc2n1CC(=O)OC]

To a solution of tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (500 mg, 1.16 mmol, INT B1 step 5) in DMF (5 mL) was added DIEA (450 mg, 3.48 mmol) and methyl 2-bromoacetate (267 mg, 1.74 mmol). The mixture was stirred at 25° C. for 12 h. then diluted with H₂O (20 mL), and extracted with EtOAc (20 mL×3). The combined organic layers were washed with brine (20 mL), dried over Na₂SO₄, filtered and concentrated under reduced pressure. The residue was purified by flash chromatography on a silica gel column eluting with EtOAc/PE (60%) to afford the title compound (400 mg, 86% yield) as a brown solid. LCMS calc. for C₂₄H₃₅N₆O₆ [M+H]⁺: m/z=503.2; Found: 502.5.

### Step 2: methyl 2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetate

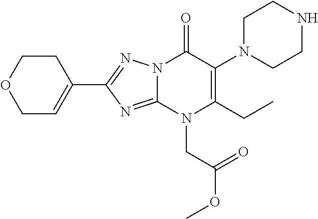 [SMILES: CCc1c(N2CCNCC2)c(=O)n2nc(C3=CCOCC3)nc2n1CC(=O)OC]

To a solution of tert-butyl 4-(2-(3,6-dihydro-2H-pyran-4-yl)-5-ethyl-4-(2-methoxy-2-oxoethyl)-7-oxo-4,7-dihydro-[1,2,4]triazolo[1,5-a]pyrimidin-6-yl)piperazine-1-carboxylate (500 mg, 0.99 mmol) in DCM (5 mL) was added TFA (5 mL). The mixture was stirred at 25° C. for 2 h. The solvent was removed under reduced pressure to afford the title compound (400 mg, crude) as a brown solid. LCMS calc. for C₁₉H₂₇N₆O₄ [M+H]⁺: m/z=403.2; Found: 403.4.

### Step 3: methyl 2-(2-(3,6-dihydro-2H-pyran-4-yl)-5-ethyl-6-(4-((2-fluorobenzoyl)glycyl)piperazin-1-yl)-7-oxo-[1,2,4]triazolo[1,5-a]pyrimidin-4(7H)-yl)acetate

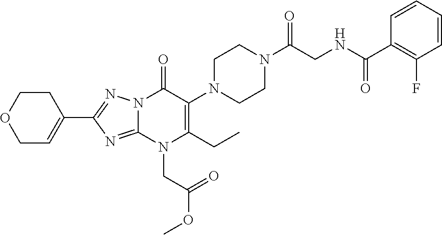 [SMILES: CCc1c(N2CCN(C(=O)CNC(=O)c3ccccc3F)CC2)c(=O)n2nc(C3=CCOCC3)nc2n1CC(=O)OC]

To a solution of methyl 2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetate (400 mg, 0.99 mmol) in DMF (5 mL) was added (2-fluorobenzoyl)glycine (392 mg, 1.99 mmol), HOBt (201 mg, 1.49 mmol), EDCI (286 mg, 1.49 mmol) and DIEA (386 mg, 2.98 mmol). The mixture was stirred at 25° C. for 2 h. and then concentrated. The residue was purified by Prep-HPLC on a C18 column eluting with MeCN/H₂O (50%) to afford the title compound (300 mg, 52% yield) as an off-white solid. LCMS calc. for C₂₈H₃₃FN₇O₆ [M+H]⁺: m/z=582.2; Found: 582.3.

### Step 4: 2-(2-(3,6-dihydro-2H-pyran-4-yl)-5-ethyl-6-(4-((2-fluorobenzoyl)glycyl)piperazin-1-yl)-7-oxo-[1,2,4]triazolo[1,5-a]pyrimidin-4(7H)-yl)acetic acid

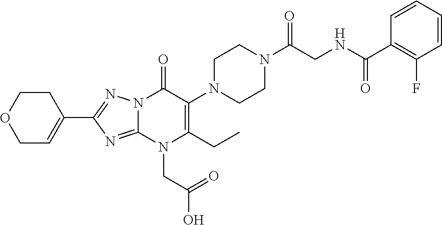 [SMILES: CCc1c(N2CCN(C(=O)CNC(=O)c3ccccc3F)CC2)c(=O)n2nc(C3=CCOCC3)nc2n1CC(=O)O]

To a solution of methyl 2-(2-(3,6-dihydro-2H-pyran-4-yl)-5-ethyl-6-(4-((2-fluorobenzoyl)glycyl)piperazin-1-yl)-7-oxo-[1,2,4]triazolo[1,5-a]pyrimidin-4(7H)-yl)acetate (300 mg, 0.52 mmol) in THF (3 mL) was added LiOH·H₂O (43 mg, 1.03 mmol) in H₂O (1 mL). The mixture was stirred at 25° C. for 0.5 h. and diluted with H₂O (10 mL), adjusted to pH=2-3 with HCl aq. (1 N).

Solids were collected by filtration and purified by Prep-HPLC on a C18 column eluting with MeCN/H₂O (30%) to afford the title compound (200 mg, 68% yield) as a brown solid. LCMS calc. for C₂₇H₃₁FN₇O₆ [M+H]⁺: m/z=568.2; Found: 568.3.

### Example A: WRN ATPase Assay

WRN ATPase assay was initiated to evaluate potential inhibitory effect of compounds for ATP hydrolysis activity of WRN protein. WRN [UniPro: Q14191 (WRN_HUMAN), N500-C946] was expressed and purified in insect system and stored at −80° C. in aliquots. FORKF DNA was annealed with equal amounts of OLIGOA-BHQ2 (TTTTTTTTTTTTTTTTTTTTTTTTTTTTTTCGTACCC-GATGTGTTCGTTC-BHQ2) and OLIGOB-TAMRA (TAMRAGAACGAACACATCGGGTACG-TTTTTTTTTTTTTTTTTTTTTTTTTTTTTT) in equal amounts, boiling for 5 min. and allowing the oligos to slowly cool to r.t. in the presence of 50 mM NaCl. ADP quantity produced from ATP hydrolysis by WRN catalytic reaction was measured using a commercially available ADP-Glo Assay (ADP-Glo™ Kinase Assay from Promega, 10000 assays, #V9102). The assay buffer was comprised of 30 mM HEPES pH7.4 (Gibco, 15630-080), 40 mM KCl (Sigma, P9541-500G), 5% Glycerol (Sigma, G7757-1L), 8 mM MgCl₂ (Invitrogen, AM9530G), 0.1 mg/mL BSA (PerkinElmer, CR84-100). Compounds dissolved in DMSO (Sigma, D8418) were plated into a 96-well intermediate plate (BioFil, VWP-032-096) for 3-fold serial titration for 10 points using DMSO, and well-to-well transfer the titrated compounds into another 96-well intermediate plate including assay buffer in advance. Shake the 96-well intermediate plate at 600 rpm for 10 min to fully mix. Specifically, a 4× working mixture of WRN was diluted to a concentration of 8 nM in assay buffer. A 2× working mixture of FORKF (Joshua A. S., Tomasz K., etc., 2019) DNA and ATP was diluted to a concentration of 30 nM and 120 μM, respectively. 2.5 μL titrated compounds from 96-well intermediate plate and 2.5 μL 4×WRN working mixture were delivered into 384-well assay plate (PerkinElmer, 6007290) using electronic pipettor. After centrifuging at 1000 rpm and shaking at 500 rpm, the assay plate was pre-incubated for 30 min. Then, 5 μL 2×FORKF DNA and ATP working mixture was dispensed into assay plate. Plate was sealed, centrifuged and incubated for an additional 40 min. at 37° C. The reaction was stopped with the addition of 10 μL of the first ADP-Glo reagent and incubated for 40 min. to remove the excess amount of ATP. Afterwards, 20 μL of ATP detection reagent was added and incubated for 30 min. before reading. Luminescence output was recorded using BMG CLARIO star plus reader. Each concentration of compound was tested in duplicates in the assay plate. Average luminescence signal of high control (Wells with 1% DMSO) was calculated as High Control (HC). Average luminescence signal of low control (Wells with no WRN protein) was calculated as Low control (LC).

$$\% inhibition = 100 - 100 * ( {Signal}_{cmpd} - {Signal}_{Ave\_LC} ) / ( {Signal}_{Ave\_HC} - {Signal}_{Ave\_LC} ) .$$

IC₅₀ values were determined by fitting the data to the standard 4 parameters with Hill Slope using GraphPad Prism software. IC₅₀ data is proved below in Table 13: a “+” denotes an IC₅₀ value of >1 μM, a “++” denotes an IC₅₀ value of 0.1 μM<IC₅₀<=1 μM, and a “+++” denotes an IC₅₀ value of <=0.1 μM.

TABLE 13
WRN enzymatic activity assay

| | ATPase | | ATPase | | ATPase |
|---|---|---|---|---|---|
| | assay | | assay | | assay |
| Ex. # | (IC₅₀) | Ex. # | (IC₅₀) | Ex. # | (IC₅₀) |
| 1 | ++ | 2 | ++ | 3 | +++ |
| 4 | +++ | 5 | + | 6 | ++ |
| 7 | +++ | 8 | + | 9 | ++ |
| 10 | | 11 | ++ | 12 | |
| 13 | | 14 | +++ | 15 | |
| 16 | | 17 | +++ | 18 | ++ |
| 19 | + | 20 | +++ | 21 | |
| 22 | +++ | 23 | + | 24 | |
| 25 | +++ | 26 | | 27 | ++ |
| 28 | ++ | 29 | +++ | 30 | ++ |
| 31 | ++ | 32 | ++ | 33 | |
| 34 | | 35 | ++ | 36 | |
| 37 | +++ | 38 | | 39 | |
| 40 | | 41 | +++ | 42 | +++ |
| 43 | ++ | 44 | ++ | 45 | +++ |
| 46 | ++ | 47 | +++ | 48 | +++ |
| 49 | +++ | 50 | +++ | 51 | |
| 52 | | 53 | +++ | 54 | ++ |
| 55 | +++ | 56 | +++ | 57 | +++ |
| 58 | +++ | 59 | ++ | 60 | ++ |
| 61 | +++ | 62 | +++ | 63 | +++ |
| 64 | ++ | 65 | ++ | 66 | + |
| 67 | +++ | 68 | ++ | 69 | ++ |
| 70 | ++ | 71 | +++ | 72 | +++ |
| 73 | +++ | 74 | +++ | 75 | ++ |
| 76 | ++ | 77 | +++ | 78 | +++ |
| 79 | ++ | 80 | ++ | 81 | +++ |
| 82 | +++ | 83 | +++ | 84 | +++ |
| 85 | +++ | 86 | ++ | 87 | +++ |
| 88 | +++ | 89 | +++ | 90 | +++ |
| 91 | +++ | 92 | +++ | 93 | +++ |
| 94 | +++ | 95 | +++ | 96 | +++ |
| 97 | +++ | 98 | +++ | 99 | +++ |
| 100 | +++ | 101 | +++ | 102 | +++ |
| 103 | +++ | 104 | ++ | 105 | ++ |
| 106 | ++ | 107 | +++ | 108 | +++ |
| 109 | +++ | 110 | +++ | 111 | +++ |
| 112 | +++ | 113 | +++ | 114 | +++ |
| 115 | +++ | 116 | +++ | 117 | +++ |
| 118 | +++ | 119 | +++ | 120 | ++ |
| 121 | +++ | 122 | +++ | 123 | +++ |
| 124 | +++ | 125 | ++ | 126 | +++ |
| 127 | | 128 | ++ | 129 | +++ |
| 130 | +++ | 131 | ++ | 132 | +++ |
| 133 | +++ | 134 | +++ | 135 | |
| 136 | +++ | 137 | | 138 | +++ |
| 139 | | 140 | | 141 | + |
| 142 | | 143 | ++ | 144 | ++ |
| 145 | | 146 | ++ | 147 | +++ |
| 148 | +++ | 149 | +++ | 150 | +++ |
| 151 | | 152 | ++ | 153 | ++ |
| 154 | +++ | 155 | | 156 | |
| 157 | +++ | 158 | | 159 | |
| 160 | | 161 | +++ | 162 | ++ |
| 163 | +++ | 164 | | 165 | +++ |
| 166 | ++ | 167 | +++ | 168 | +++ |
| 169 | ++ | 170 | ++ | 171 | ++ |
| 172 | | 173 | ++ | 174 | +++ |
| 175 | | 176 | ++ | 177 | +++ |
| 178 | +++ | 179 | +++ | 180 | +++ |
| 181 | +++ | 182 | +++ | 183 | +++ |
| 184 | +++ | 185 | +++ | 186 | +++ |
| 187 | +++ | 188 | +++ | 189 | +++ |
| 190 | +++ | 191 | +++ | 192 | +++ |
| 193 | +++ | 194 | +++ | 195 | +++ |
| 196 | +++ | 197 | +++ | 198 | +++ |
| 199 | +++ | 200 | +++ | 201 | +++ |
| 202 | ++ | 203 | +++ | 204 | +++ |
| 205 | +++ | 206 | +++ | 207 | ++ |
| 208 | +++ | 209 | +++ | 210 | +++ |
| 211 | +++ | 212 | ++ | 213 | +++ |
| 214 | +++ | 215 | +++ | 216 | +++ |
| 217 | +++ | 218 | +++ | 219 | +++ |
| 220 | +++ | 221 | +++ | 222 | +++ |
| 223 | +++ | 224 | +++ | 225 | +++ |
| 226 | +++ | 227 | +++ | 228 | +++ |
| 229 | ++ | 230 | | 231 | +++ |
| 232 | +++ | 233 | +++ | 234 | ++ |
| 235 | ++ | 236 | ++ | 237 | ++ |
| 238 | ++ | 239 | ++ | 240 | |
| 241 | ++ | 242 | | 243 | |
| 244 | +++ | 245 | | 246 | ++ |
| 247 | | 248 | +++ | 249 | +++ |
| 250 | +++ | 251 | +++ | 252 | +++ |
| 253 | +++ | 254 | +++ | 255 | +++ |
| 256 | +++ | 257 | ++ | 258 | +++ |
| 259 | ++ | 260 | +++ | 261 | +++ |
| 262 | ++ | 263 | +++ | 264 | +++ |
| 265 | +++ | 266 | +++ | 267 | +++ |
| 268 | +++ | 269 | +++ | 270 | +++ |
| 271 | ++ | 272 | +++ | 273 | ++ |
| 274 | +++ | 275 | +++ | 276 | +++ |
| 277 | +++ | 278 | +++ | 279 | +++ |
| 280 | +++ | 281 | +++ | 282 | +++ |
| 283 | ++ | 284 | +++ | 285 | +++ |
| 286 | +++ | 287 | +++ | 288 | +++ |
| 289 | +++ | 290 | +++ | 291 | +++ |
| 292 | +++ | 293 | +++ | 294 | |
| 295 | ++ | 296 | ++ | 297 | +++ |
| 298 | +++ | 299 | ++ | 300 | ++ |
| 301 | ++ | 302 | | 303 | +++ |
| 304 | +++ | 305 | +++ | 306 | +++ |
| 307 | ++ | 308 | +++ | 309 | |
| 310 | +++ | 311 | +++ | 312 | ++ |
| 313 | ++ | 314 | ++ | 315 | |
| 316 | | 317 | | 318 | +++ |
| 319 | +++ | 320 | +++ | 321 | ++ |
| 322 | +++ | 323 | ++ | 324 | +++ |
| 325 | +++ | 326 | ++ | 327 | ++ |
| 328 | | 329 | ++ | 330 | +++ |
| 331 | +++ | 332 | +++ | 333 | +++ |
| 334 | +++ | 335 | ++ | 336 | ++ |
| 337 | +++ | 338 | +++ | 339 | |
| 340 | ++ | 341 | +++ | 342 | +++ |
| 343 | +++ | 344 | ++ | 345 | ++ |
| 346 | +++ | 347 | +++ | 348 | +++ |
| 349 | +++ | 350 | +++ | 351 | +++ |
| 352 | ++ | 353 | +++ | 354 | +++ |
| 355 | +++ | 356 | + | 357 | ++ |
| 358 | ++ | 359 | ++ | 360 | + |
| 361 | +++ | 362 | ++ | 363 | + |
| 364 | +++ | 365 | ++ | 366 | +++ |
| 367 | +++ | 368 | +++ | 369 | +++ |
| 370 | +++ | 371 | +++ | 372 | +++ |
| 373 | +++ | 374 | +++ | 375 | +++ |
| 376 | +++ | 377 | +++ | 378 | +++ |
| 379 | +++ | 380 | ++ | 381 | +++ |
| 382 | +++ | 383 | +++ | 384 | +++ |
| 385 | +++ | 386 | +++ | | |

### Example B: WRN Unwinding Assay

WRN unwinding assay was initiated to evaluate potential inhibitory effect of compounds for WEN helicase activity. WRN [UniPro: Q14191 (WRN_HUMAN), N500-C946] was expressed and purified in insect system and stored at −80° C. in aliquots. FORKF DNA was annealed with equal amounts of OLIGOA-BHQ2 (TTTTTTTTTTTTTTTTTTTTTTTTTTTTTTCGTACCCGATGTGTTCGTTC-BHQ2) and OLIGOB-TAMRA (TAMRA-GAACGAACACATCGGGTACGTTTTTTTTTTTTTTTTTTTTTTTTTTTTTT) in equal amounts, boiling for 5 min. and allowing the oligos to slowly cool to r.t. in the presence of 50 mM NaCl. The assay buffer was comprised of 30 mM HEPES pH 7.4 (Gibco, 15630-080), 40 mM KCl (Sigma, P9541-500G), 5% Glycerol (sigma, G7757-1L), 8 mM MgCl₂ (Invitrogen, AM9530G), 0.1 mg/ml BSA (PerkinElmer, CR84-100). Compounds dissolved in DMSO (Sigma, D8418) were plated into a 96-well intermediate plate (BioFil, VWP-032-096) for 3-fold serial titration for 10 points using DMSO, and well-to-well transfer the titrated compounds into another 96-well intermediate plate including assay buffer in advance. Shake the 96-well intermediate plate at 600 rpm for 10 min. to fully mix. Specifically, a 3× working mixture of WRN was diluted to a concentration of 60 nM in assay buffer. A 3× working mixture of FORKF DNA and ATP was diluted to a concentration of 37.5 nM and 600 M, respectively. 5 μL titrated compounds from 96-well intermediate plate and 5 ML 3×WRN working mixture were delivered into 384-well assay plate (PerkinElmer, 6008260) using electronic pipettor. After centrifuging at 1000 rpm and shaking at 500 rpm, the assay plate was pre-incubated for 30 min. Then, 5 ML 3×FORKF DNA and ATP working mixture was dispensed into assay plate. Plate was sealed, centrifuged and incubated for an additional 40 min. at 25° C. Fluorescence (Ex558 nm/Em586 nm) output was recorded using BMG CLARIO star plus reader. Each concentration of compound was tested in duplicates in the assay plate. Average fluorescence signal of high control (Wells with 1% DMSO) was calculated as High Control (HC). Average fluorescence signal of low control (Wells with no WRN protein) was calculated as Low control (LC).

$$\% inhibition = 100 - 100 * ( {Signal}_{cmpd} - {Signal}_{Ave\_LC} ) / ( {Signal}_{Ave\_HC} - {Signal}_{Ave\_LC} ) .$$

IC₅₀ values were determined by fitting the data to the standard 4 parameters with Hill Slope using GraphPad Prism software. IC₅₀ data is proved below in Table 14: a “+” denotes an IC₅₀ value of >1 μM, a “++” denotes an IC₅₀ value of 0.1 μM<IC₅₀<=1 μM, and a “+++” denotes an IC₅₀ value of <=0.1 μM.

TABLE 14
WRN unwinding assay

| | Unwinding | | Unwinding | | Unwinding |
|---|---|---|---|---|---|
| | assay | | assay | | assay |
| Ex. # | (IC₅₀) | Ex. # | (IC₅₀) | Ex. # | (IC₅₀) |
| 1 | ++ | 2 | ++ | 3 | +++ |
| 4 | +++ | 5 | + | 6 | +++ |
| 7 | ++ | 8 | + | 9 | + |
| 10 | + | 11 | + | 12 | ++ |
| 13 | ++ | 14 | +++ | 15 | ++ |
| 16 | ++ | 17 | +++ | 18 | ++ |
| 19 | + | 20 | +++ | 21 | + |
| 22 | +++ | 23 | + | 24 | ++ |
| 25 | +++ | 26 | + | 27 | +++ |
| 28 | ++ | 29 | +++ | 30 | +++ |
| 31 | +++ | 32 | +++ | 33 | ++ |
| 34 | ++ | 35 | ++ | 36 | ++ |
| 37 | ++ | 38 | ++ | 39 | ++ |
| 40 | + | 41 | +++ | 42 | +++ |
| 43 | +++ | 44 | ++ | 45 | +++ |
| 46 | +++ | 47 | +++ | 48 | +++ |
| 49 | +++ | 50 | ++ | 51 | + |
| 52 | + | 53 | ++ | 54 | ++ |
| 55 | +++ | 56 | +++ | 57 | +++ |
| 58 | +++ | 59 | +++ | 60 | ++ |
| 61 | +++ | 62 | +++ | 63 | +++ |
| 64 | +++ | 65 | ++ | 66 | + |
| 67 | +++ | 68 | ++ | 69 | +++ |
| 70 | ++ | 71 | +++ | 72 | +++ |
| 73 | +++ | 74 | +++ | 75 | +++ |
| 76 | +++ | 77 | +++ | 78 | +++ |
| 79 | +++ | 80 | +++ | 81 | +++ |
| 82 | +++ | 83 | +++ | 84 | +++ |
| 85 | +++ | 86 | +++ | 87 | +++ |
| 88 | +++ | 89 | +++ | 90 | ++ |
| 91 | ++ | 92 | +++ | 93 | +++ |
| 94 | +++ | 95 | +++ | 96 | +++ |
| 97 | +++ | 98 | +++ | 99 | +++ |
| 100 | +++ | 101 | +++ | 102 | +++ |
| 103 | +++ | 104 | +++ | 105 | +++ |
| 106 | +++ | 107 | +++ | 108 | +++ |
| 109 | +++ | 110 | +++ | 111 | +++ |
| 112 | +++ | 113 | +++ | 114 | +++ |
| 115 | +++ | 116 | +++ | 117 | +++ |
| 118 | +++ | 119 | +++ | 120 | +++ |
| 121 | +++ | 122 | +++ | 123 | +++ |
| 124 | +++ | 125 | +++ | 126 | +++ |
| 127 | ++ | 128 | +++ | 129 | +++ |
| 130 | +++ | 131 | +++ | 132 | +++ |
| 133 | +++ | 134 | +++ | 135 | +++ |
| 136 | ++ | 137 | + | 138 | +++ |
| 139 | + | 140 | + | 141 | + |
| 142 | ++ | 143 | + | 144 | +++ |
| 145 | ++ | 146 | ++ | 147 | +++ |
| 148 | +++ | 149 | +++ | 150 | +++ |
| 151 | ++ | 152 | +++ | 153 | +++ |
| 154 | +++ | 155 | + | 156 | + |
| 157 | +++ | 158 | ++ | 159 | ++ |
| 160 | + | 161 | +++ | 162 | ++ |
| 163 | +++ | 164 | ++ | 165 | +++ |
| 166 | +++ | 167 | +++ | 168 | +++ |
| 169 | +++ | 170 | ++ | 171 | ++ |
| 172 | ++ | 173 | ++ | 174 | +++ |
| 175 | ++ | 176 | ++ | 177 | +++ |
| 178 | +++ | 179 | +++ | 180 | +++ |
| 181 | +++ | 182 | +++ | 183 | +++ |
| 184 | +++ | 185 | +++ | 186 | +++ |
| 187 | +++ | 188 | +++ | 189 | +++ |
| 190 | +++ | 191 | +++ | 192 | +++ |
| 193 | +++ | 194 | +++ | 195 | +++ |
| 196 | +++ | 197 | +++ | 198 | +++ |
| 199 | +++ | 200 | +++ | 201 | +++ |
| 202 | ++ | 203 | +++ | 204 | +++ |
| 205 | +++ | 206 | +++ | 207 | ++ |
| 208 | +++ | 209 | +++ | 210 | +++ |
| 211 | +++ | 212 | ++ | 213 | +++ |
| 214 | +++ | 215 | +++ | 216 | +++ |
| 217 | +++ | 218 | +++ | 219 | +++ |
| 220 | +++ | 221 | +++ | 222 | +++ |
| 223 | +++ | 224 | ++ | 225 | +++ |
| 226 | +++ | 227 | +++ | 228 | +++ |
| 229 | +++ | 230 | ++ | 231 | +++ |
| 232 | +++ | 233 | +++ | 234 | +++ |
| 235 | ++ | 236 | ++ | 237 | ++ |
| 238 | ++ | 239 | ++ | 240 | + |
| 241 | ++ | 242 | ++ | 243 | ++ |
| 244 | ++ | 245 | +++ | 246 | +++ |
| 247 | ++ | 248 | +++ | 249 | +++ |
| 250 | +++ | 251 | +++ | 252 | +++ |
| 253 | +++ | 254 | +++ | 255 | +++ |
| 256 | +++ | 257 | ++ | 258 | +++ |
| 259 | +++ | 260 | +++ | 261 | +++ |
| 262 | +++ | 263 | +++ | 264 | +++ |
| 265 | +++ | 266 | +++ | 267 | +++ |
| 268 | +++ | 269 | +++ | 270 | +++ |
| 271 | +++ | 272 | +++ | 273 | +++ |
| 274 | +++ | 275 | +++ | 276 | +++ |
| 277 | +++ | 278 | +++ | 279 | +++ |
| 280 | +++ | 281 | +++ | 282 | +++ |
| 283 | +++ | 284 | +++ | 285 | +++ |
| 286 | +++ | 287 | +++ | 288 | +++ |
| 289 | +++ | 290 | +++ | 291 | +++ |
| 292 | +++ | 293 | +++ | 294 | ++ |
| 295 | + | 296 | ++ | 297 | +++ |
| 298 | +++ | 299 | ++ | 300 | ++ |
| 301 | ++ | 302 | ++ | 303 | +++ |
| 304 | +++ | 305 | +++ | 306 | +++ |
| 307 | +++ | 308 | +++ | 309 | ++ |
| 310 | +++ | 311 | +++ | 312 | ++ |
| 313 | ++ | 314 | + | 315 | ++ |
| 316 | ++ | 317 | ++ | 318 | +++ |
| 319 | +++ | 320 | +++ | 321 | +++ |
| 322 | +++ | 323 | ++ | 324 | +++ |
| 325 | +++ | 326 | +++ | 327 | ++ |
| 328 | ++ | 329 | ++ | 330 | +++ |
| 331 | +++ | 332 | +++ | 333 | +++ |
| 334 | +++ | 335 | +++ | 336 | ++ |
| 337 | +++ | 338 | +++ | 339 | ++ |
| 340 | ++ | 341 | +++ | 342 | +++ |
| 343 | +++ | 344 | ++ | 345 | ++ |
| 346 | +++ | 347 | +++ | 348 | +++ |
| 349 | +++ | 350 | +++ | 351 | +++ |
| 352 | ++ | 353 | +++ | 354 | +++ |
| 355 | +++ | 356 | + | 357 | ++ |
| 358 | ++ | 359 | ++ | 360 | + |
| 361 | +++ | 362 | +++ | 363 | + |
| 364 | +++ | 365 | +++ | 366 | +++ |
| 367 | +++ | 368 | +++ | 369 | +++ |
| 370 | +++ | 371 | +++ | 372 | +++ |
| 373 | ++ | 374 | +++ | 375 | +++ |
| 376 | +++ | 377 | +++ | 378 | +++ |
| 379 | +++ | 380 | ++ | 381 | +++ |
| 382 | +++ | 383 | +++ | 384 | +++ |
| 385 | +++ | 386 | +++ | | |

### Example: Cell Viability Assay

Cell viability studies were conducted in colon carcinoma cell lines SW48 which is MSI-H cell lines, and the MSS cell line SW620 was included as a control. Cells were cultured in RPMI 1640 (Hyclone, SH3080901B) supplemented with 10% FBS (AusGeneX, FBS500-S) and 1% penicillin-streptomycin (Gibco, 15140122). Cells were seeded in 96-well cell culture plates (PerkinElmer, 6005680) at a density of 1000 cells/well for SW48 cell and 500 cells/well for SW620 cell. Compounds dissolved in DMSO were plated in duplicate using a multichannel pipette, and tested on a 9-point 3-fold serial dilution. The final concentration of DMSO was 0.2% in all wells. Cells were incubated for 6 days in a 37° C., 5% CO₂ humidified incubator. Cell viability was measured using the Cell Titer-Glo reagent (Promega, Catalog #: G7573) according to the manufacturer's instructions, and luminescence signal was measured with a multimode plate reader (BMG CLARIO star plus). Average values of 0.2% DMSO-treated wells on each plate were calculated as high control (HC), while average values of only medium in each plate was calculated as low control (LC). The percent inhibition was calculated using the formula: % inhibition=100-100*(Signal_{cmpd}−Signal_{Ave_LC})/(Signal_{Ave_HC}−Signal_{Ave_LC}).

IC₅₀ values were determined by fitting the data to the standard 4 parameters with Hill Slope using GraphPad Prism software. IC₅₀ data is proved: a “+” denotes an IC₅₀ value of >1 μM, a “++” denotes an IC₅₀ value of 0.5 M<IC₅₀<=1 μM, and a “+++” denotes an IC₅₀ value of <=0.5 μM.

Although the present invention has been comprehensively described through its embodiments, it is worth noting that various changes and modifications are obvious to those skilled in the art. Such changes and modifications should be included in the scope of the appended claims of the present invention.

What is claimed is:

1. A compound of formula (I) or a pharmaceutically acceptable salt, stereoisomer, solvate, tautomer, isotopic variant, prodrug, N-oxide or deuterated compound thereof, wherein: 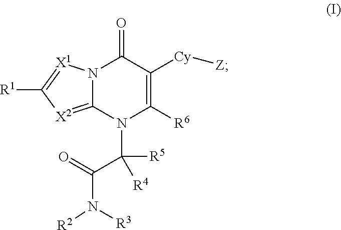
X¹ and X² are same or different, each is independently N or CR⁷;
Z is H, ring A, —C(O)R, —C(═NR¹⁰)R¹¹, —C(═NR¹⁰)NR^{11B}R^{11C}, —C(O)NR^{11B}R^{11C}, —C(O)OR^{11A}, —NR^{11C}C(O)R¹¹, —S(O)₂NR^{11B}R^{11C}, —NR^{11C}S(O)₂R¹¹, —S(O)₂NR^{11C}C(O)R¹¹, —C(O)NR^{11C}S(O)₂R¹¹, —C(O)NR^{11C}S(O)(═NR¹⁰)R¹¹, —S(O)R¹¹, —S(O)₂R¹¹, —S(O)(═NR¹⁰)R¹¹, —NR^{11C}C(O)NR^{11B}R^{11C}, —NR^{11C}C(O)OR^{11A}, or —C(O) C(O)NR^{11B}R^{11C};
ring A is C₃-C₁₀ cycloalkyl, 4-10 membered heterocycloalkyl, C₆-C₁₀ aryl or 5-10 membered heteroaryl; wherein, ring A is optionally substituted by 1, 2, 3, 4, 5 or 6 substituents R⁸;
R is C₁-C₈ alkyl, C₂-C₈ alkenyl, C₂-C₈ alkynyl, C₃-C₁₀ cycloalkyl, 4-10 membered heterocycloalkyl, C₆-C₁₀ aryl or 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl or 5-10 membered heteroaryl is optionally substituted by 1, 2, 3, 4, 5 or 6 substituents R⁹;
Cy is C₅-C₁₀ cycloalkylene, 5-14 membered heterocycloalkylene, C₆-C₁₀ arylene, or 5-10 membered heteroarylene; wherein, the Cy is optionally substituted by 1, 2, 3, 4 or 5 substituents independently selected from R¹²;
R¹ is halo, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, OR^{A}, SR^{A}, or NR^{C}R^{D}; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b};
R² is C₁-C₆ alkyl, C₃-C₁₀ cycloalkyl, 4-10 membered heterocycloalkyl, C₆-C₁₀ aryl or 5-10 membered heteroaryl; wherein, the C₁-C₆ alkyl, C₃-C₁₀ cycloalkyl, 4-10 membered heterocycloalkyl, C₆-C₁₀ aryl or 5-10 membered heteroaryl is optionally substituted by 1, 2, 3, 4, or 5 substituents independently selected from R²⁴;
each R^{2A} is independently D, halo, CN, NO₂, N₃, SF₅, oxo, C₁-C₆ alkyl, C₂-C₆ alkenyl, C₂-C₆ alkynyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, OH, N₃, oxo, C₁-C₆ alkyl, C₁-C₆ haloalkyl, OC₁-C₆ alkyl, OC₁-C₆ haloalkyl, OC₁-C₆ alkyl-OH, C₃-C₅ cycloalkyl, or 4-5 membered heterocycloalkyl;
R³ is independently H, D, C(O)R^{B}, C₁-C₆ alkyl, C₁-C₆ haloalkyl, C₃-C₆ cycloalkyl or 4-6 membered heterocycloalkyl; or
R² and R³ together with the atom to which they are attached form 4-10 membered heterocycloalkyl or 5-10 membered heteroaryl; wherein, the 4-10 membered heterocycloalkyl or 5-10 membered heteroaryl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, OC₁-C₄ haloalkyl, C₃-C₅ cycloalkyl, or 4-5 membered heterocycloalkyl;
R⁴ and R⁵ are each independently H, D, halo, C₁-C₃ alkyl, C₁-C₃ haloalkyl, C₁-C₃ alkyl-OH, C₁-C₃ alkyl-CN, —C₁-C₃ alkyl-O—C₁-C₃ alkyl, or —C₁-C₃ alkyl-O—C₁-C₃ haloalkyl; or
R⁴ and R⁵ together with the atom to which they are attached form C₃-C₅ cycloalkyl or 4-5 membered heterocycloalkyl; wherein, the C₃-C₅ cycloalkyl or 4-5 membered heterocycloalkyl is optionally substituted with 1, 2, or 3 substituents independently selected from D, halo, OH, oxo, CN, —NH₂, —NH(C₁-C₄ alkyl), —N(C₁-C₄ alkyl)₂, C₁-C₄ alkyl, C₁-C₄ haloalkyl, OC₁-C₄ alkyl, or OC₁-C₄ haloalkyl;
R⁶ is H, D, OR^{A}, NR^{C}R^{D}, SR^{A}, C(O)R^{B}, C₁-C₄ alkyl, C₂-C₄ alkenyl, C₂-C₄ alkynyl, C₃-C₅ cycloalkyl or 4-5 membered heterocycloalkyl; wherein, the C₁-C₄ alkyl, C₂-C₄ alkenyl, C₂-C₄ alkynyl, C₃-C₅cycloalkyl or 4-5 membered heterocycloalkyl is optionally substituted by 1, 2 or 3 substituents independently selected from D, halo, CN, N₃, NO₂, C₁-C₄ alkyl, C₁-C₄ haloalkyl, OH, OC₁-C₄ alkyl, OC₁-C₄ haloalkyl; or
R⁵ and R⁶ together with the atoms to which they are attached form a 5-7 membered heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, C₃-C₅ cycloalkyl or 4-5 membered heterocycloalkyl;
R⁷ is independently H, D, halo, CN, OH, NH₂, C₁-C₃ alkyl, C₁-C₃ haloalkyl, OC₁-C₃ alkyl, OC₁-C₃ haloalkyl, C₃-C₈ cycloalkyl or 4-5 membered heterocycloalkyl;
each R⁸ is independently H, D, halo, CN, NO₂, N₃, oxo, C₁-C₆ alkyl, C₂-C₆ alkenyl, C₂-C₆ alkynyl, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, oxo, NR^{c}R^{d}, OR^{a}, SR^{a}, C(O)R^{b}, C₁-C₄ alkyl, C₁-C₄ haloalkyl, C₃-C₅ cycloalkyl or 4-5 membered heterocycloalkyl;
each R⁹ is independently H, D, halo, CN, NO₂, N₃, oxo, C₁-C₆ alkyl, C₂-C₆ alkenyl, C₂-C₆ alkynyl, C₁-C₄ alkyl-O—C₁-C₄ alkyl-Si(C₁-C₄ alkyl)₃, —NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, OP(O)OR^{E}OR^{F}, or Cy¹; wherein, the C₁-C₆ alkyl, C₂-C₆ alkenyl, C₂-C₆ alkynyl is optionally substituted by 1, 2, 3, 4 or 5 substituents independently selected from D, halo, CN, NO₂, N₃, OXO, NR^{c}R^{d}, OR^{a}, SR^{a}, C(O)R^{b}, C₁-C₄alkyl, C₁-C₄ haloalkyl, C₃-C₅ cycloalkyl or 4-5 membered heterocycloalkyl;
each R¹⁰ is independently H, D, CN, OH, OMe, or C₁-C₄ alkyl optionally substituted by 1, 2, 3, 4 or 5 substituents independently selected from D, halo, CN, —OH, —O—C₁-C₄ alkyl, —OC₁-C₄ haloalkyl, NH₂, —NH(C₁-C₄ alkyl), or —N(C₁-C₄ alkyl)₂;
each R¹¹ is independently C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₅ alkenyl, C₂-C₅ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³; or
R¹⁰ and R¹¹ together with the atoms to which they are attached form 5-6 membered heteroaryl, 5-6 membered partially unsaturated heterocycloalkyl; wherein, the 5-6 membered heteroaryl or 5-6 membered partially unsaturated heterocycloalkyl is optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, or SC₁-C₄ alkyl; or
each R^{11A} is independently H, D, C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³;
each R^{11B} is independently H, D, OR^{A}, C(O)R^{B}, C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, 5-10 membered heteroaryl; wherein, the C₁-C₈ alkyl, C₂-C₈ alkenyl, C₂-C₈ alkynyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from R¹³;
each R^{11C} is independently H, D, C₁-C₄ alkyl, C₂-C₄ alkenyl, C₂-C₄ alkynyl, C₃-C₇ cycloalkyl, or 4-7 membered heterocycloalkyl; wherein, the C₁-C₄ alkyl, C₂-C₄ alkenyl, C₂-C₄ alkynyl, C₃-C₇ cycloalkyl or 4-7 membered heterocycloalkyl is optionally substituted by 1, 2, 3, 4, 5 or 6 substituents independently selected from D, halo, OH, oxo, CN, NO₂, N₃, C₁-C₄ alkyl, C₁-C₄ haloalkyl, OC₁-C₄ alkyl, or OC₁-C₄ haloalkyl; or
R^{11B} and R^{11C} together with the atom to which they are attached form a 4-10 membered heterocycloalkyl optionally substituted with 1, 2, or 3 substituents independently selected from D, halo, OH, NH₂, oxo, CN, C₁-C₄ alkyl, C₁-C₄ haloalkyl, OC₁-C₄ alkyl, OC₁-C₄ haloalkyl, NHC₁-C₄ alky, N(C₁-C₄ alkyl)₂, C₃-C₅ cycloalkyl or 4-5 membered heterocycloalkyl;
each R¹² is independently H, D, halo, CN, oxo, NO₂, C₁-C₆ alkyl, C₂-C₆ alkenyl, C₂-C₆ alkynyl, C₃-C₆ cycloalkyl, 4-6 membered heterocycloalkyl, NR^{C}R^{D}, OR^{A}, SR^{A}, C(O)R^{B}, S(O)R^{B}, S(O)₂R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, NR^{C}S(O)₂R^{B}, or NR^{C}S(O)₂NR^{C}R^{D}; wherein, the C₁-C₆ alkyl, C₂-C₆ alkenyl, C₂-C₆ alkynyl, C₃-C₆ cycloalkyl, 4-6 membered heterocycloalkyl is optionally substituted by 1, 2, 3, 4, 5 or 6 substituents independently selected from D, halo, CN, NO₂, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b};
wherein, two R¹² together with the same ring carbon atom to which they are attached form oxo, C₃-C₄ cycloalkyl, 4 membered heterocycloalkyl having I heteroatom selected from Si, N, O or S;
wherein, the C₃-C₄ cycloalkyl, 4 membered heterocycloalkyl is optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl;
wherein, two R¹² together with the atoms to which they are attached form C₃-C₆ cycloalkyl, 4-6 membered heterocycloalkyl, phenylene or 5-6 membered heteroarylene; wherein, the C₃-C₆ cycloalkyl, 4-6 membered heterocycloalkyl, phenylene or 5-6 membered heteroarylene is optionally substituted by 1, 2, 3 or 4 substituents independently selected from D, halo, OH, C₁-C₆ alkyl, C₁-C₆ haloalkyl, —O—C₁-C₆ alkyl, or —OC₁-C₆ haloalkyl;
each R¹³ is independently H, D, halo, CN, NO₂, N₃, OXO, SF₅, C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl, C₀-C₆ alkyl-5-10 membered heteroaryl, C₁-C₄ alkyl-O—C₁-C₄ alkyl-Si(C₁-C₄ alkyl) 3, NR^{C}R^{D}, OR^{A}, SR^{A}, C(O)R^{B}, C(O)OR^{A}, OC(O)R^{B}, C(O)NR^{C}R^{D}, NR^{C}C(O)R^{B}, OC(O)NR^{C}R^{D}, OC(O)OR^{A}, NR^{C}C(O)NR^{C}R^{D}, NR^{C}C(O)OR^{A}, C(═NR^{C})NR^{C}R^{D}, NR^{D}C(═NR^{C})NR^{C}R^{D}, NR^{D}C(═NR^{C})R^{B}, S(O)R^{B}, S(O)NR^{C}R^{D}, S(O)₂R^{B}, S(O)₂NR^{C}R^{D}, NR^{C}S(O)₂R^{B}, S(O)(═NR^{B})R^{B}, NR^{C}S(O)₂NR^{C}R^{D}, NR^{C}S(O)(═NR^{B})R^{B}, B(OR^{E})(OR^{F}), P(O)R^{E}R^{F}, P(O)OR^{E}OR^{F}, or OP(O)OR^{E}OR^{F}; wherein, the C₁-C₆ alkyl, C₂-C₆ alkenyl, C₂-C₆ alkynyl, C₀-C₆ alkyl-C₃-C₆ cycloalkyl, C₀-C₆ alkyl-4-6 membered heterocycloalkyl, C₀-C₆ alkyl-C₆-C₁₀ aryl, C₀-C₆ alkyl-5-10 membered heteroaryl is optionally substituted by 1, 2, 3, 4, 5, 6 substituents independently selected from D, halo, CN, NO₂, SF₅, oxo, C₁-C₆ alkyl, C₁-C₆ haloalkyl, C₁-C₆ alkyl-OH, C₁-C₆ alkyl-CN, NR^{c}R^{d}, OR^{a}, SR^{a}, NHOR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{c}S(O)₂R^{b}, or S(O)₂NR^{c}R^{d};
Cy¹ is C₃-C₁₀cycloalkyl, 4-10 membered heterocycloalkyl, C₆-C₁₀ aryl or 5-10 membered heteroaryl; wherein, the C₃-C₁₀ cycloalkyl, 4-10 membered heterocycloalkyl, C₆-C₁₀ aryl or 5-10 membered heteroaryl is optionally substituted by 1, 2, 3 or 4 substituents independently selected from D, halo, CN, NO₂, C₁-C₆ alkyl, C₁-C₆ haloalkyl, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}, S(O)R^{b}, S(O)₂R^{b}, NR^{C}S(O)₂R^{b}, or S(O)₂NR^{a}R^{d};
each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, C₁-C₄ alkyl, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, B(OR^{e})(OR^{f}), C(═NR^{C})NR^{c}R^{d}, NR^{c}C(═NR^{C})NR^{c}R^{d}, NR^{c}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{C}S(O)₂R^{b}, S(O)₂NR^{c}R^{d}, NR^{c}S(O)₂NR^{c}R^{d}, or NR^{c}S(O)(═NR^{b})R^{b};
each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, C(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{C}S(O)₂R^{b}, S(O)₂NR^{c}R^{d}, NR^{c}S(O)₂NR^{c}R^{d}, or B(OR^{e})(OR^{f});
R^{C} and R^{D} are each independently H, D, C₁-C₆ alkyl, C₂-C₆ alkenyl, C₂-C₆ alkynyl, C₃-C₇cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₇ cycloalkyl, 4-7 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, or 3 substituents independently selected from D, OH, CN, halo, oxo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄alkyl, OC₂-C₄ alkyl-O—C₁-C₄ haloalkyl, C₁-C₄ alkyl-O—C₁-C₄ alkyl, C₁-C₄ alkyl-O—C₁-C₄ haloalkyl, SF₅, OC(O)NR^{c}R^{d}, NR^{c}R^{d}, NR^{c}C(O)R^{b}, S(O)NR^{c}R^{d}, S(O)₂R^{b}, NR^{C}S(O)₂R^{b}, S(O)₂NR^{c}R^{d}, NR^{C}S(O)₂NR^{c}R^{d}, or B(OR^{e})(OR^{f}); or
R^{C} and R^{D} together with the N atom to which they are attached form a 4-7 membered heterocycloalkyl optionally substituted with 1, 2, or 3 substituents independently selected from D, OH, oxo, CN, —NH₂, —NH(C₁-C₄ alkyl), —N(C₁-C₄ alkyl)₂, halo, C₁-C₄ alkyl, C₁-C₄ haloalkyl, C₁-C₄ cyanoalkyl, OC₁-C₄ alkyl, OC₁-C₄ haloalkyl, OC₂-C₄ alkylOH, OC₂-C₄ alkyl-O—C₁-C₄ alkyl, or OC₂-C₄ alkyl-O—C₁-C₄ haloalkyl;
R^{a} and R^{a1} are each independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein, the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, halo, CN, —NH₂, —NH(C₁-C₄ alkyl), —N(C₁-C₄ alkyl)₂, C₁-C₄ alkyl, C₁-C₄ alkoxy, C₁-C₄ haloalkyl, or C₁-C₄ haloalkoxy;
R^{b} and R^{b1} are each independently H, D, C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein, the C₁-C₄ alkyl, C₂-C₄ alkenyl, C₂-C₄ alkynyl, phenyl, C₃-C₇ cycloalkyl, 5-6 membered heteroaryl, 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₆-C₁₀ aryl, C₃-C₁₀ cycloalkyl, 5-10 membered heteroaryl, or 4-10 membered heterocycloalkyl;
R^{c} and R^{d} are each independently H, D, C₁-C₄ alkyl, C₁-C₄ haloalkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀ cycloalkyl, 4-10 membered heteroaryl-4-10 membered heterocycloalkyl, 4-10 membered heteroaryl-C₆-C₁₀ aryl, or bi(4-10 membered heteroaryl);
wherein the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 4-10 membered heteroaryl-C₃-C₁₀ alkyl, C₃-C₁₀ cycloalkyl-C₆-C₁₀ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-4-10 membered heteroaryl, bi(C₆-C₁₀ aryl), 4-10 membered heteroaryl-C₃-C₁₀ cycloalkyl, 4-10 membered heteroaryl-4-10 membered heterocycloalkyl, 4-10 membered heteroaryl-C₆-C₁₀ aryl, or bi(4-10 membered heteroaryl) is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—; or
R^{c} and R^{d} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
R^{E} and R^{e} are each independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl;
R^{F} and R^{f} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, or 4-10 membered heterocycloalkyl;
when 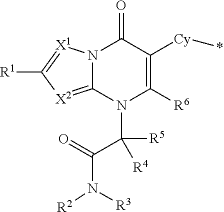
is 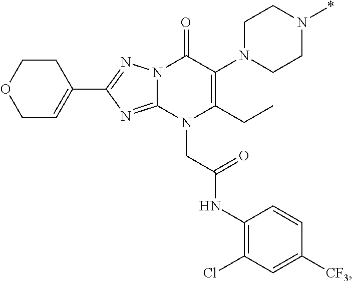 [SMILES: *N1CCN(c2c(CC)n(CC(=O)Nc3ccc(C(F)(F)F)cc3Cl)c3nc(C4=CCOCC4)nn3c2=O)CC1]
and Z is not 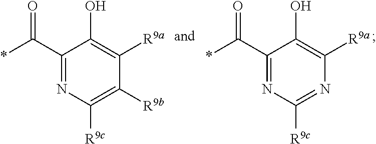
wherein, R^{9a} is H, F, Cl, or CH₃, OCF₃; R^{9b} is H, Cl, or CH₃; R^{9c} is H, or CH₃.

2. The compound of claim 1, wherein, the moiety 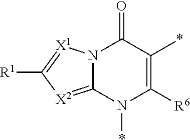
has the structure of 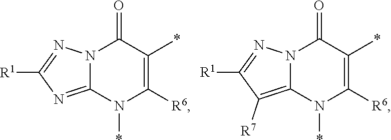
and R⁷ is H, D, halo, CN, C₁-C₃ alkyl.

3. The compound of claim 1, wherein, Cy is

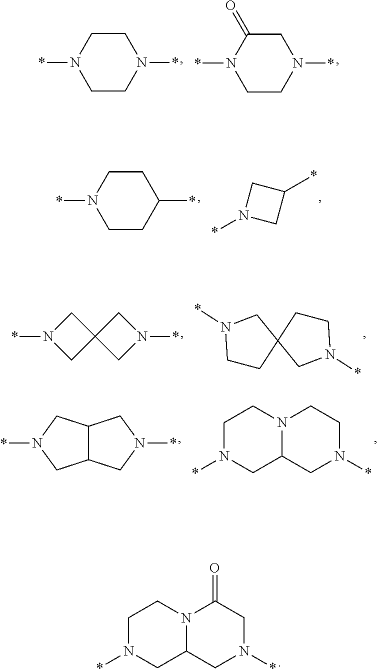

4. The compound of claim 1, wherein, R¹ is (i) halo, OR^{A}, SR^{A}, or NR^{C}R^{D}; or (ii) C₁-C₆ alkyl, C₃-C₁₀ cycloalkyl, 4-10 membered heterocycloalkyl, C₆-C₁₀ aryl, 5-10 membered heteroaryl; wherein, the C₁-C₆ alkyl, C₃-C₁₀ cycloalkyl, 4-10 membered heterocycloalkyl, C₆-C₁₀ aryl, or 5-10 membered heteroaryl is optionally substituted by 1, 2, 3, 4, 5 or 6 substituents independently selected from D, halo, CN, N₃, NO₂, SF₅, oxo, C₁-C₆ alkyl, C₁-C₆ haloalkyl, C₁-C₆ alkyl-OH, C₁-C₆ alkyl-CN, C₃-C₆ cycloalkyl, 4-6 membered heterocycloalkyl, NR^{c}R^{d}, OR^{a}, SR^{a}, C(O)R^{b}, C(O)OR^{a}, OC(O)R^{b}, C(O)NR^{c}R^{d}, NR^{c}C(O)R^{b}.

5. The compound of claim 1, wherein, R¹ is F, Cl, Br, I, CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂CN, CH₂CH₂CN, CH₂CH₂CH₂CN, 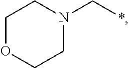
OCH₃, OCH₂CH₃, OCH₂CH₂OCH₃, or OCH₂CH₂OCH₂CH₃, SCH₃, SCH₂CH₃,

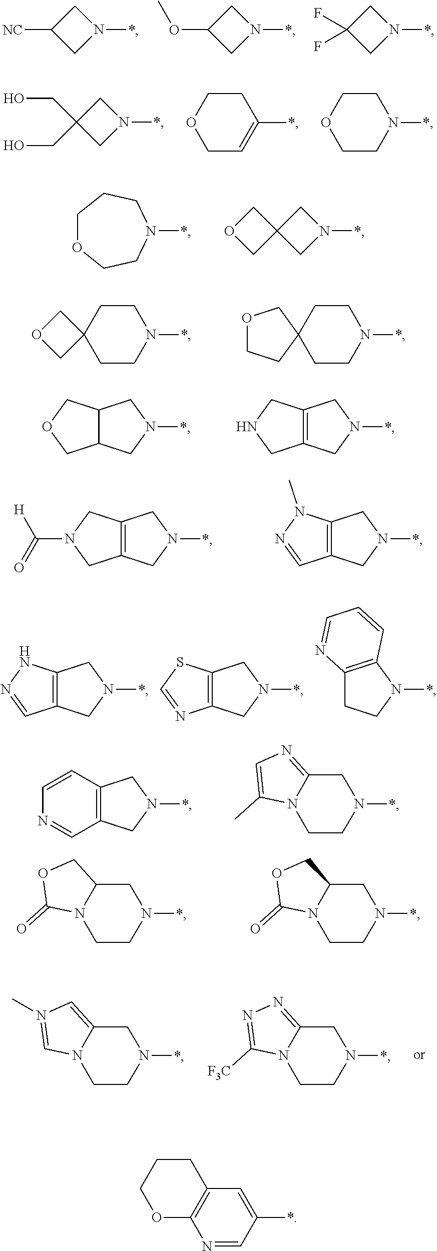

6. The compound of claim 5, wherein, R¹ is

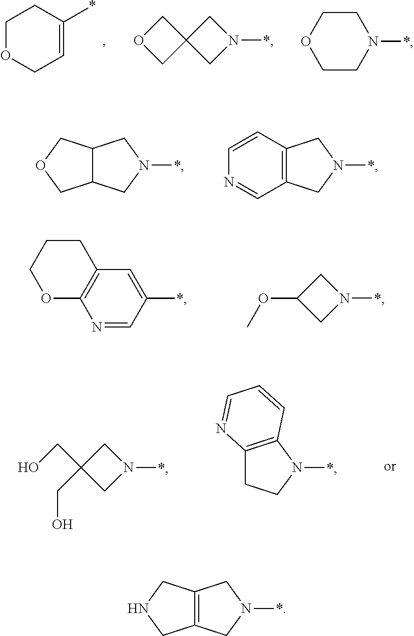

7. The compound of claim 1, wherein, R² is

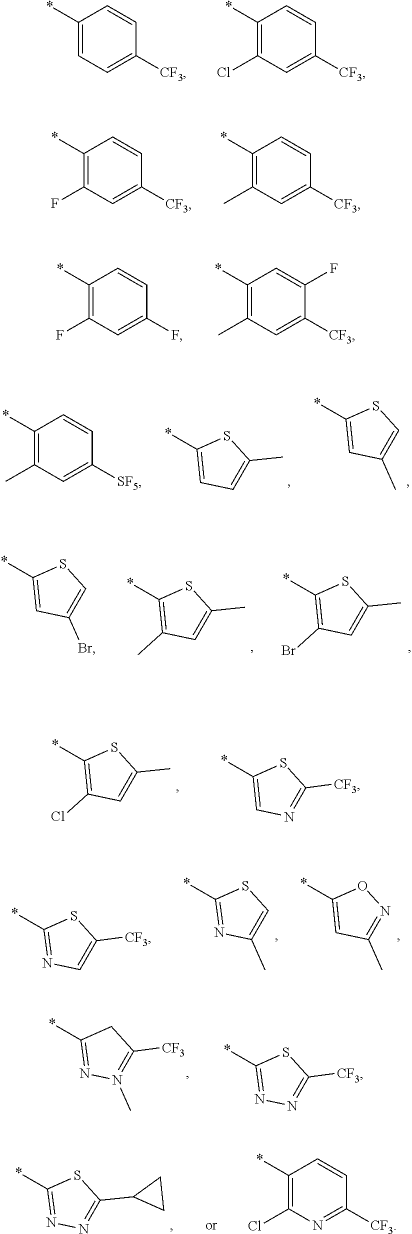

8. The compound of claim 1, wherein, R³ is H, D, CH₃.

9. The compound of claim 1, wherein, R⁴ is H or D, and R⁵ is H or D.

10. The compound of claim 1, wherein, R⁶ is H, D, OH, OCH₃, CH₃, or CH₂CH₃.

11. The compound of claim 1, wherein, Z is —C(O)NHCH₂CH₃, —C(O)N(CH₃)₂, S(O)₂NH₂, S(O)₂NHEt, —S(O)₂CH₂CH₃,

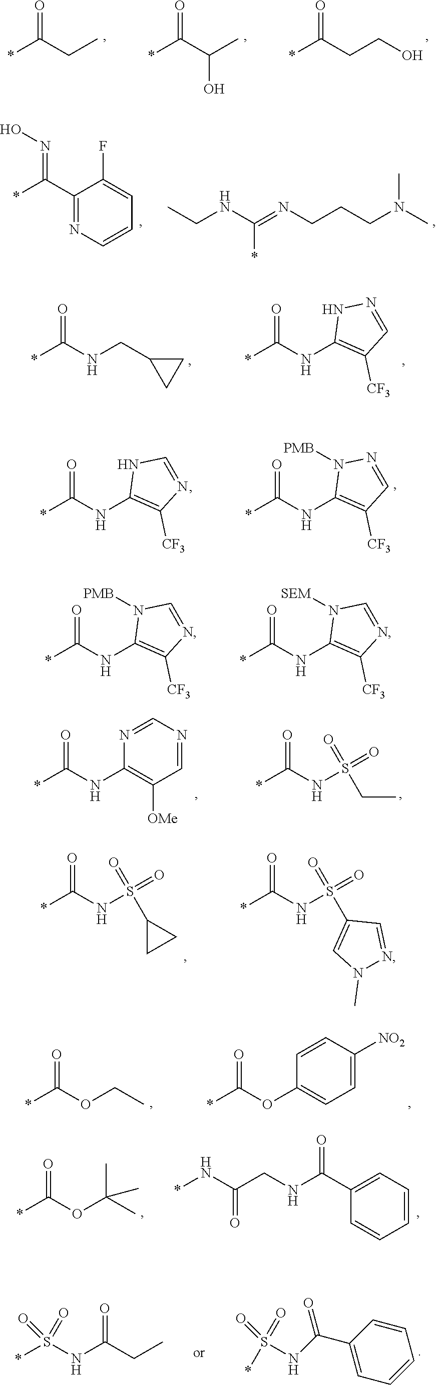

12. The compound of claim 1, wherein, the compounds of Formula (I) are represented by compounds of Formula (IIa), (IIb), (IIc), (Id), (IIe), (IIf), (IIg), (IIh), (IIi), (IIj), or (IIk): 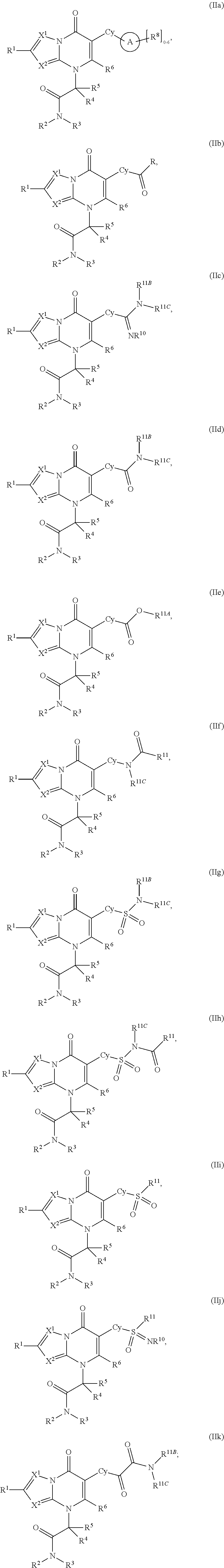
a pharmaceutically acceptable salt, stereoisomer, solvate, tantomer, isotopic variant, prodrug, N-oxide or deuterated compound thereof;
wherein, Cy, ring A, X¹, X², R, R¹, R², R³, R⁴, R⁵, R⁶, R⁸, R¹⁰, R¹¹, R^{11A}, R^{11B} and R^{11C} are defined with respect to Formula (I).

13. The compound of claim 12, wherein, the compounds of Formula (I) are represented by compounds of Formula (III): 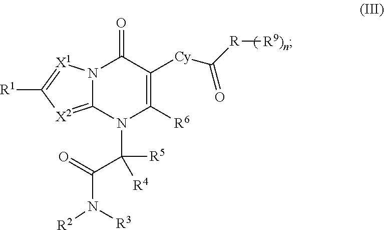
a pharmaceutically acceptable salt, stereoisomer, solvate, tautomer, isotopic variant, prodrug, N-oxide or deuterated compound thereof;
R is C₁-C₈ alkylene, C₂-C₈ alkenylene, C₂-C₈ alkynylene;
n is 1, 2, 3, 4, 5 or 6;
wherein, Cy, X¹, X², R¹, R², R³, R⁴, R⁵, R⁶ and R⁹ are defined with respect to Formula (I).

14. The compound of claim 13, wherein, the compounds of Formula (I) are represented by compounds of Formula (IVa), (IVb), (IVc), (IVd), (IVe), (IVf) or (IVg): 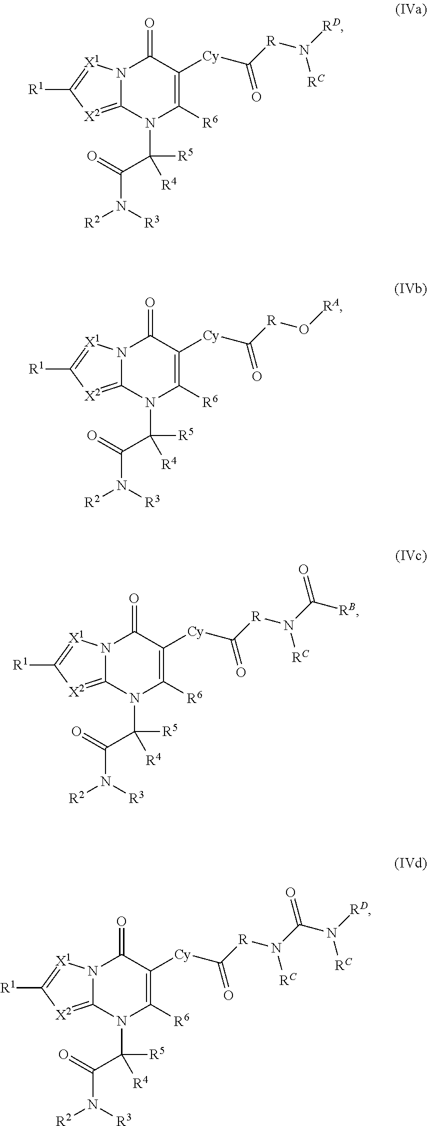 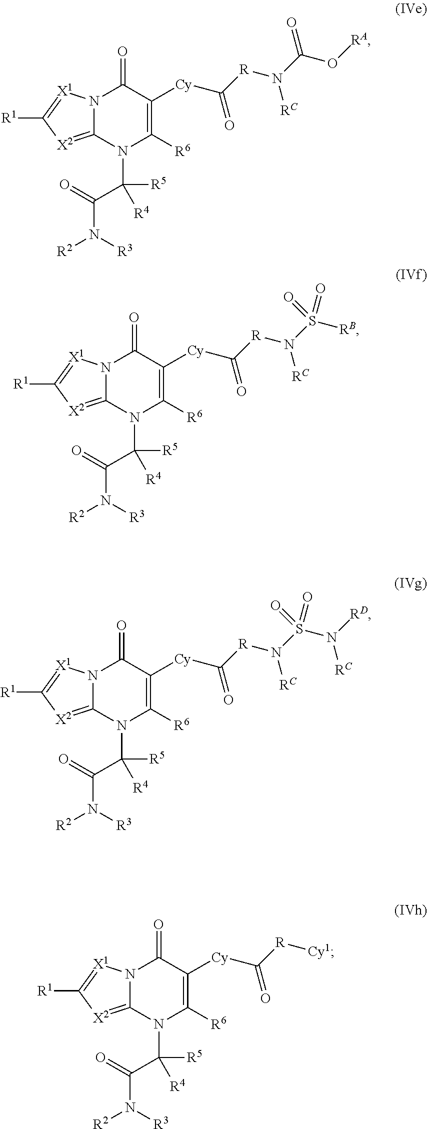
a pharmaceutically acceptable salt, stereoisomer, solvate, tautomer, isotopic variant, prodrug, N-oxide or deuterated compound thereof;
R is C₁-C₈ alkylene, C₂-C₈ alkenylene, or C₂-C₈ alkynylene; each is optionally substituted with 1, 2, 3, or 4 substituents independently selected from R⁹;
wherein, Cy, Cy¹, X¹, X², R¹, R², R³, R⁴, R⁵, R⁶, R⁹, R^{A}, R^{B}, R^{C} and R^{D} are defined with respect to Formula (I).

15. The compound of claim 13, wherein, R is —CH₂—, —CD₂-, —CHF—, —CH₂CH₂—, —CH(CH₃)—, —C(CH₃)₂—, —CF₂— or ethenylene.

16. The compound of claim 14, wherein, the compounds of Formula (I) are represented by compounds of Formula (IVc) or (IVd): 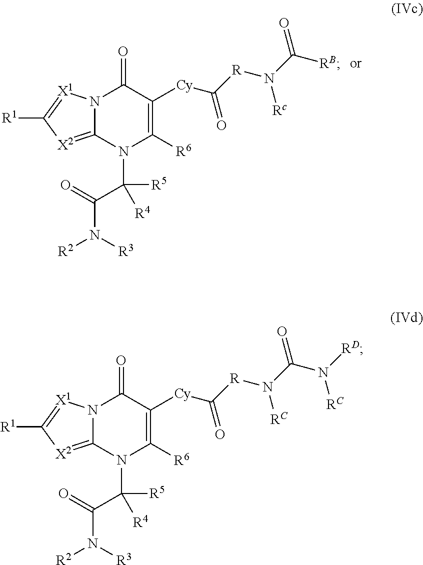
a pharmaceutically acceptable salt, stereoisomer, solvate, tautomer, isotopic variant, prodrug, N-oxide or deuterated compound thereof;
wherein, Cy, X¹, X², R, R¹, R², R³, R⁴, R⁵, R⁶, R^{B}, R^{C} and R^{D} are defined with respect to Formula (I).

17. The compound of claim 16, wherein, R^{B} is CH₃, CH₂CH₃, CH₂CH₂CH₃, CH(CH₃)₂, C(CH₃)₃, CH₂F, CHF₂, CF₃, cyclopropyl, cyclobutyl, 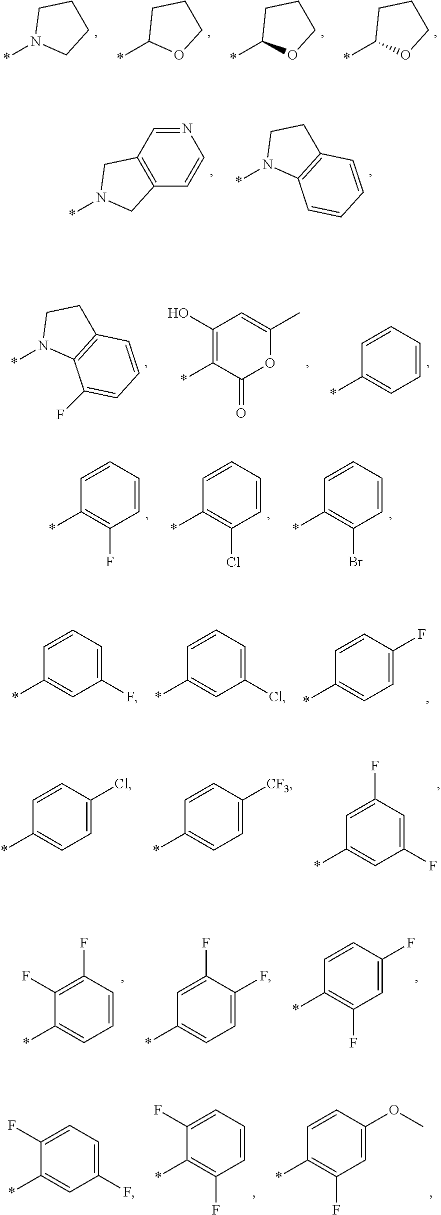 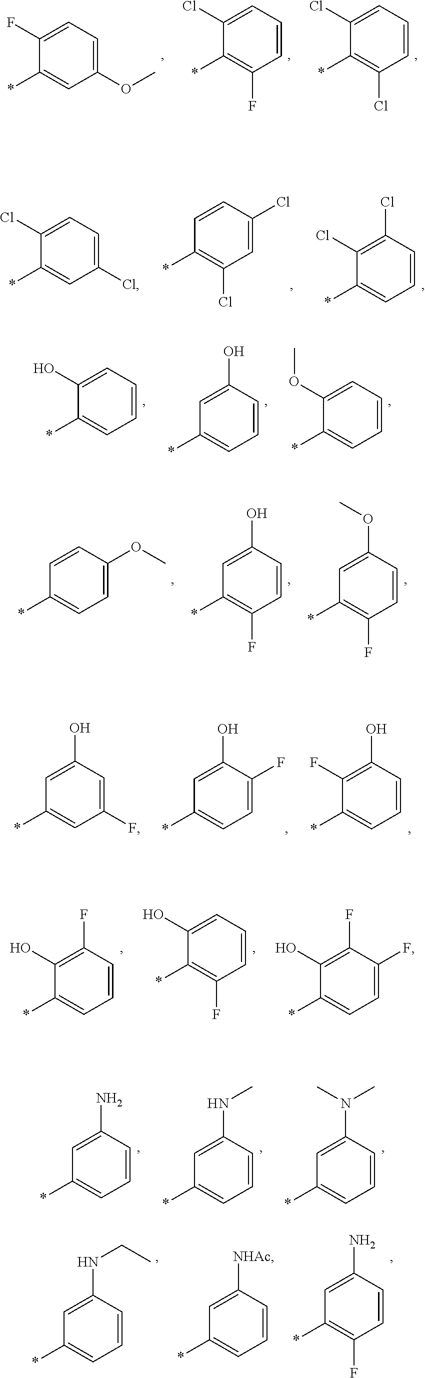 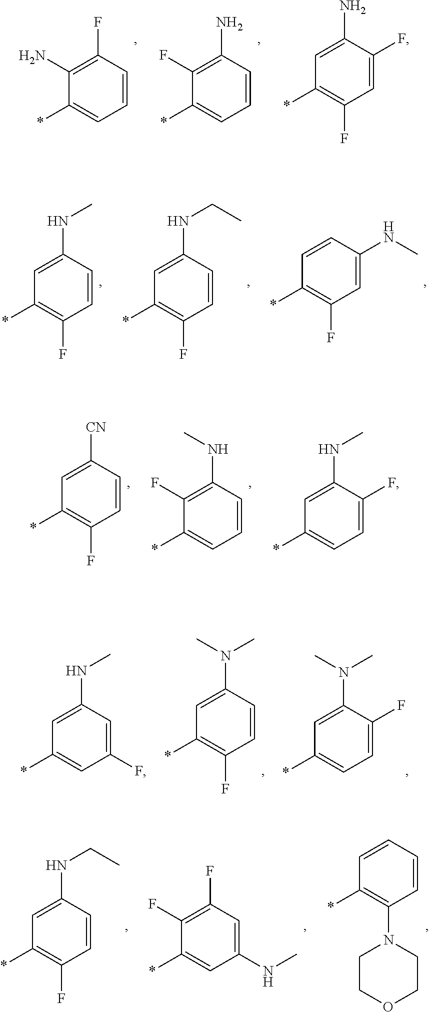 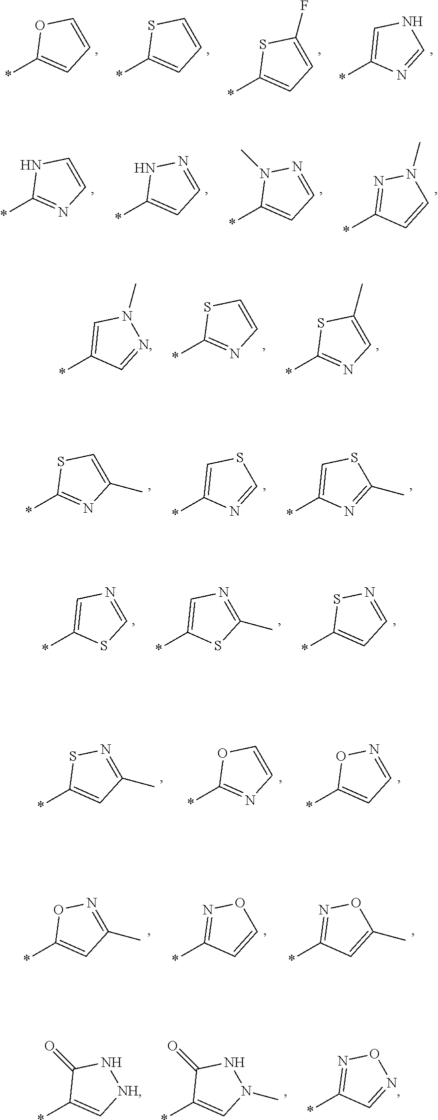 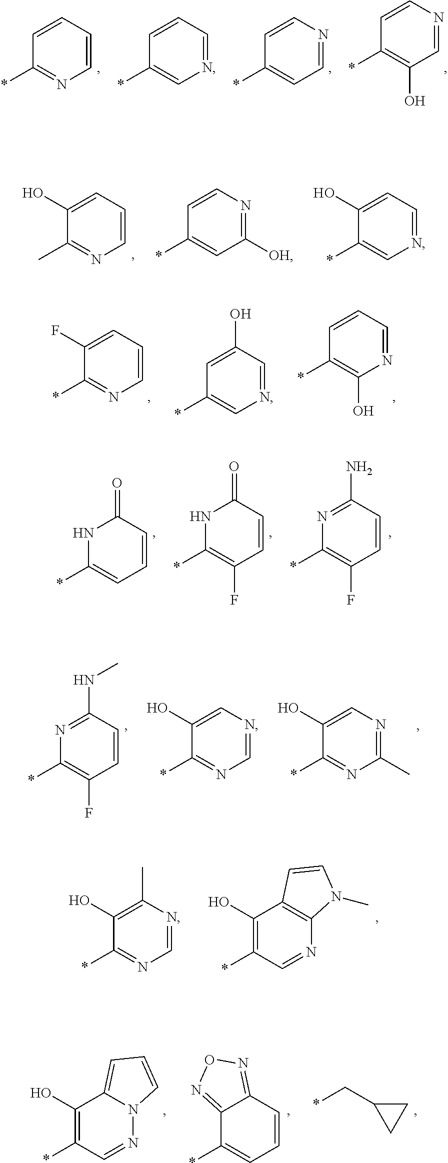 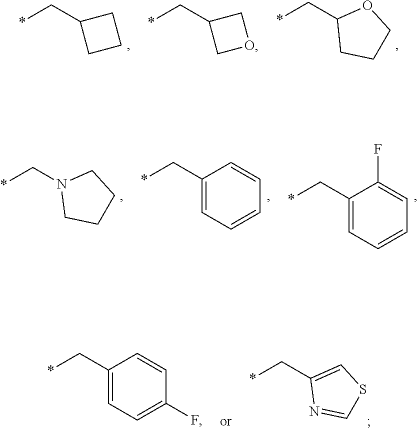
or
R^{C} is H, D, CH₃, CH₂CH₃, CD₃; or
R^{D} is H, D, CH₃, CH₂CH₃, CH₂CH₂CH₃, CH(CH₃)₂, CH₂CH₂OH, 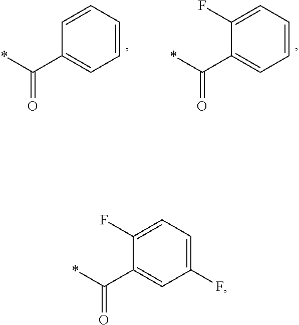 [SMILES: *C(=O)c1ccccc1 ; *C(=O)c1ccccc1F ; *C(=O)c1cc(F)ccc1F]
cyclopropyl,

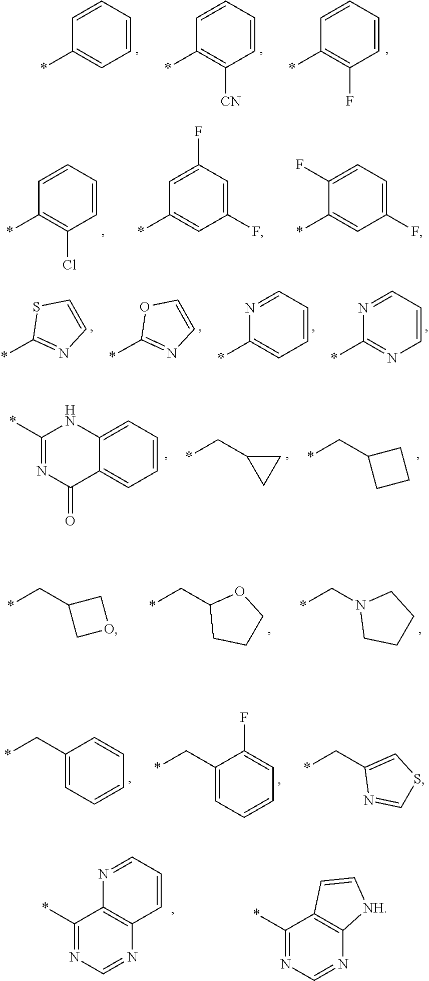

18. The compound of claim 12, wherein, the compounds of Formula (I) are represented by compounds of Formula (IIk): 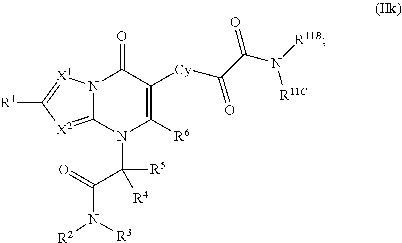
a pharmaceutically acceptable salt, stereoisomer, solvate, tautomer, isotopic variant, prodrug, N-oxide or deuterated compound thereof;
wherein, Cy, X¹, X², R¹, R², R³, R⁴, R⁵, R⁶, R^{11B} and R^{11C} are defined with respect to Formula (I).

19. The compound of claim 18, wherein, R^{11B} is H, D, OR^{A}, C₁-C₈ alkyl, C₃-C₁₀ cycloalkyl, 4-10 membered heterocycloalkyl, C₆-C₁₀ aryl, 5-10 membered heteroaryl; wherein, the C₁-C₈ alkyl, C₆-C₁₀ aryl, C₃-C₁₀ cycloalkyl, 4-10 membered heterocycloalkyl, or 5-10 membered heteroaryl is optionally substituted by 1, 2, 3, 4, 5 or 6 substituents independently selected from R¹³.

20. The compound of claim 19, wherein, R^{11B} is H, D, methyl, CH₂CH₃, CH(CH₃)₂, CH₂CF₃, OCH₃, OCH₂CH₃,

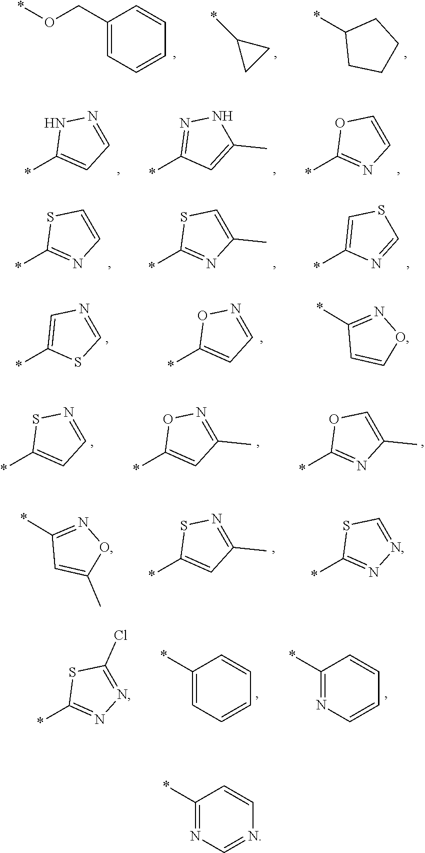

21. The compound of claim 18, wherein, R^{11C} is H, D, C₁-C₄ alkyl optionally substituted by 1, 2, 3, 4, 5 or 6 substituents independently selected from D, halo, OH, oxo, CN, NO₂, N₃, C₁-C₄ alkyl, C₁-C₄ haloalkyl, OC₁-C₄ alkyl, or OC₁-C₄ haloalkyl.

22. The compound of claim 21, wherein, R^{11C} is H, D, methyl, CH₂CH₃.

23. The compound of claim 18, wherein, R^{11B} and R^{11C} together with the atom to which they are attached form a 4-10 membered heterocycloalkyl optionally substituted with 1, 2, or 3 substituents independently selected from D, halo, OH, oxo, CN, C₁-C₄ alkyl, C₁-C₄ haloalkyl, OC₁-C₄ alkyl, OC₁-C₄ haloalkyl, C₃-C₅ cycloalkyl or 4-5 membered heterocycloalkyl.

24. The compound of claim 23, wherein, R^{11B} and R^{11C} together with the atoms to which they are attached form

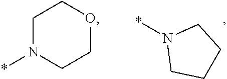 [SMILES: *N1CCOCC1 ; *N1CCCC1 ; ","]

25. The compound of claim 1, wherein, the compounds of Formula (I) are represented by compounds of Formula (V): 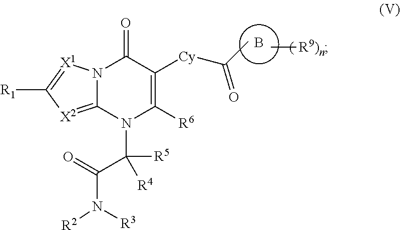
a pharmaceutically acceptable salt, stereoisomer, solvate, tautomer, isotopic variant, prodrug, N-oxide or deuterated compound thereof;
ring B is C₃-C₁₀ cycloalkyl, 4-10 membered heterocycloalkyl, C₆-C₁₀ aryl or 5-10 membered heteroaryl;
n is 1, 2, 3, 4, 5 or 6;
wherein, Cy, X¹, X², R¹, R², R³, R⁴, R⁵, R⁶ and R⁹ are defined with respect to Formula (I);
when 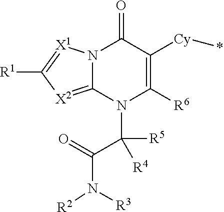
is 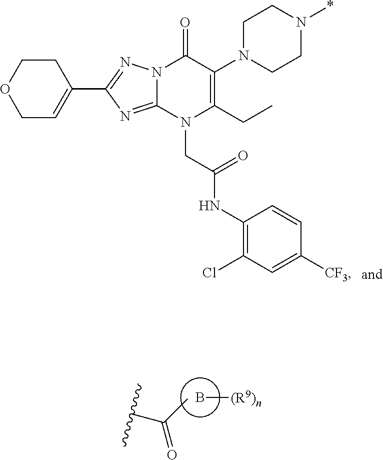
is not 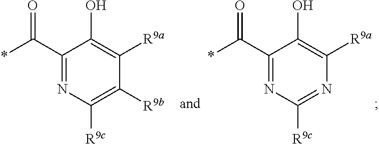
wherein, R^{9a} is H, F, Cl, or CH₃, OCF₃; R^{9b} is H, Cl, or CH₃; R^{9c}, is H, or CH₃.

26. The compound of claim 25, wherein, the moiety 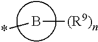
has the structure of

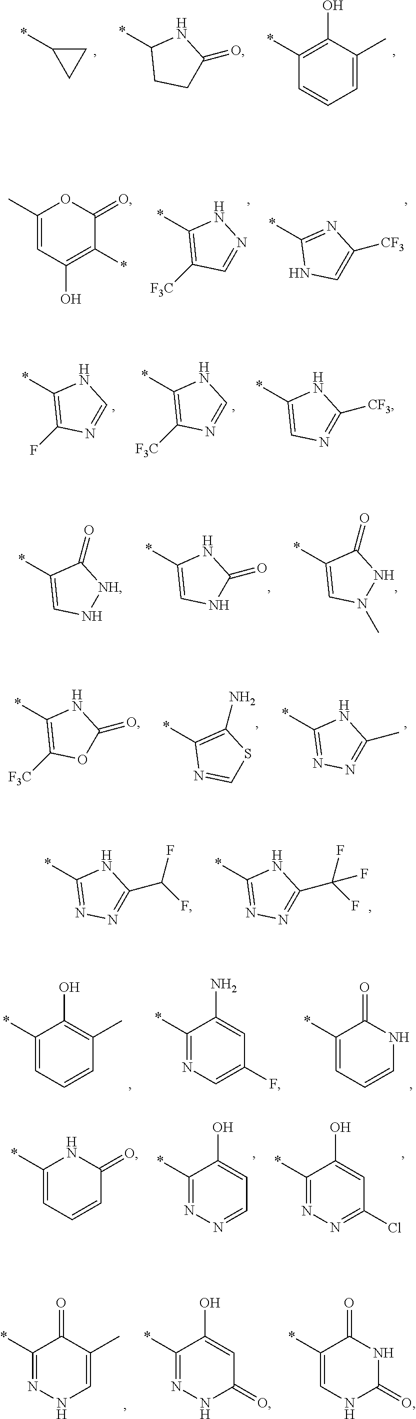

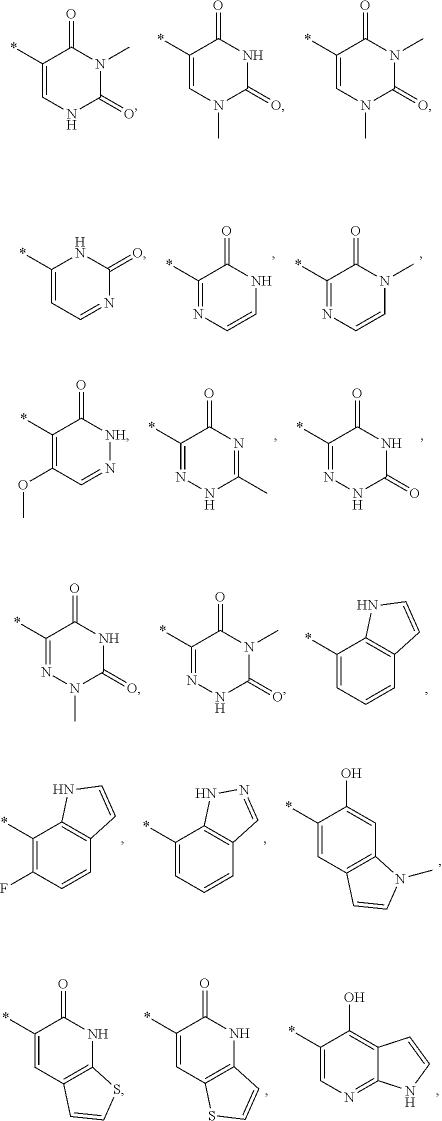

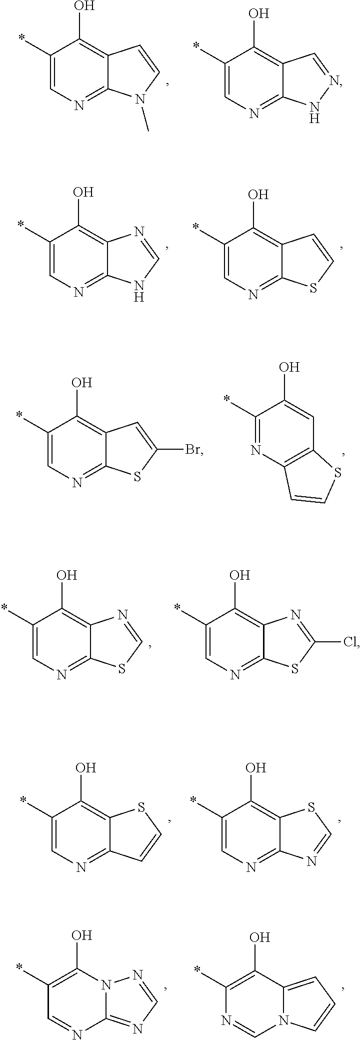

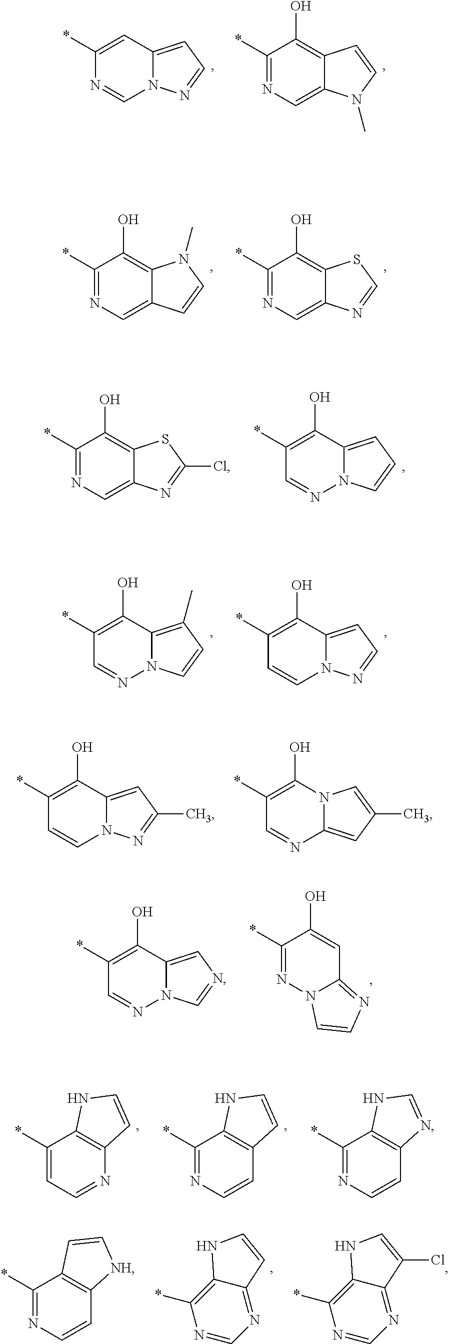

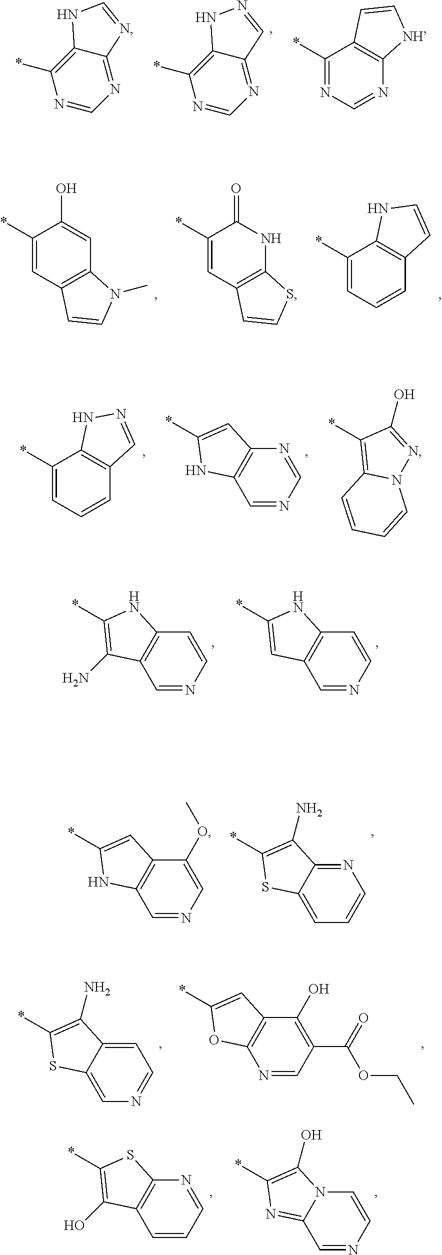

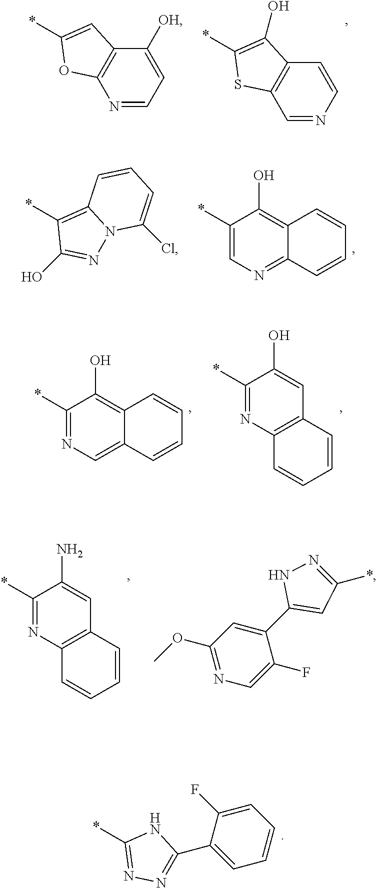

27. The compound of claim 1, wherein, the compound of Formula (I) is: 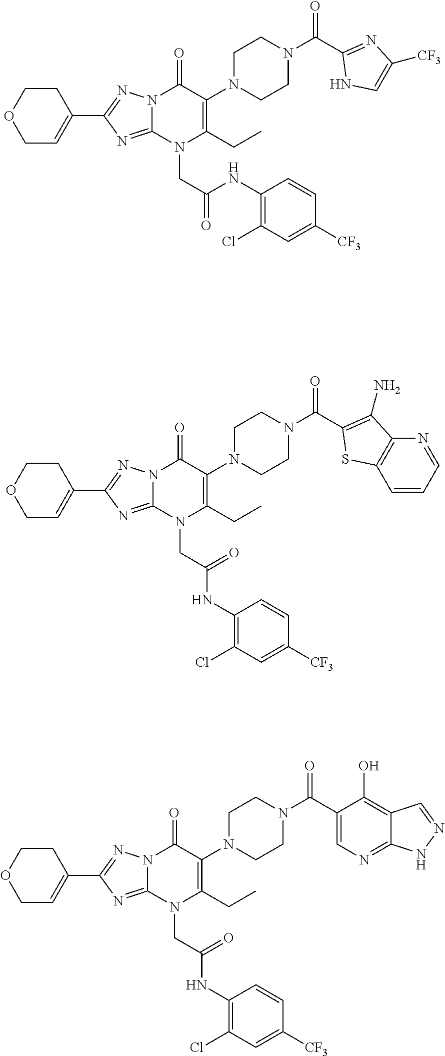 [SMILES: CCc1c(N2CCN(C(=O)c3nc(C(F)(F)F)c[nH]3)CC2)c(=O)n2nc(C3=CCOCC3)nc2n1CC(=O)Nc1ccc(C(F)(F)F)cc1Cl ; CCc1c(N2CCN(C(=O)c3sc4cccnc4c3N)CC2)c(=O)n2nc(C3=CCOCC3)nc2n1CC(=O)Nc1ccc(C(F)(F)F)cc1Cl ; CCc1c(N2CCN(C(=O)c3cnc4[nH]ncc4c3O)CC2)c(=O)n2nc(C3=CCOCC3)nc2n1CC(=O)Nc1ccc(C(F)(F)F)cc1Cl] 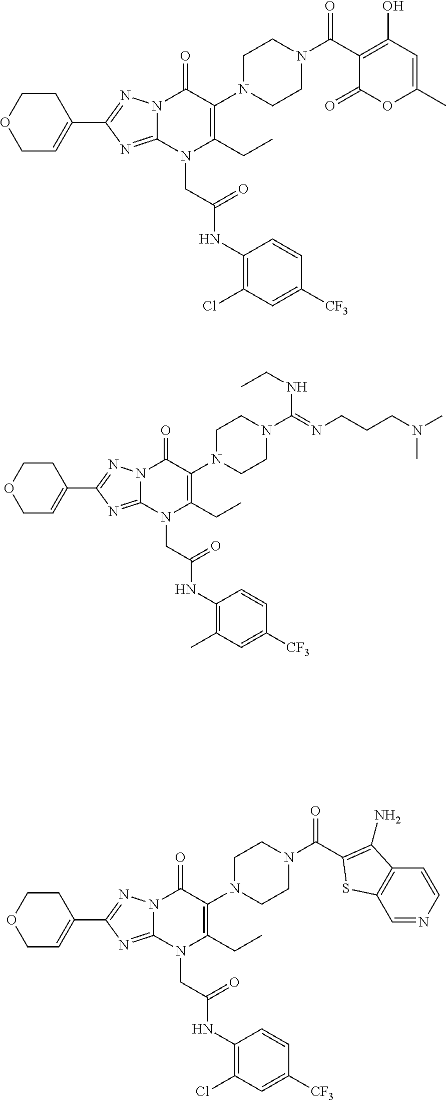 [SMILES: CCc1c(N2CCN(C(=O)c3c(O)cc(C)oc3=O)CC2)c(=O)n2nc(C3=CCOCC3)nc2n1CC(=O)Nc1ccc(C(F)(F)F)cc1Cl ; CCN/C(=N\CCCN(C)C)N1CCN(c2c(CC)n(CC(=O)Nc3ccc(C(F)(F)F)cc3C)c3nc(C4=CCOCC4)nn3c2=O)CC1 ; CCc1c(N2CCN(C(=O)c3sc4cnccc4c3N)CC2)c(=O)n2nc(C3=CCOCC3)nc2n1CC(=O)Nc1ccc(C(F)(F)F)cc1Cl] 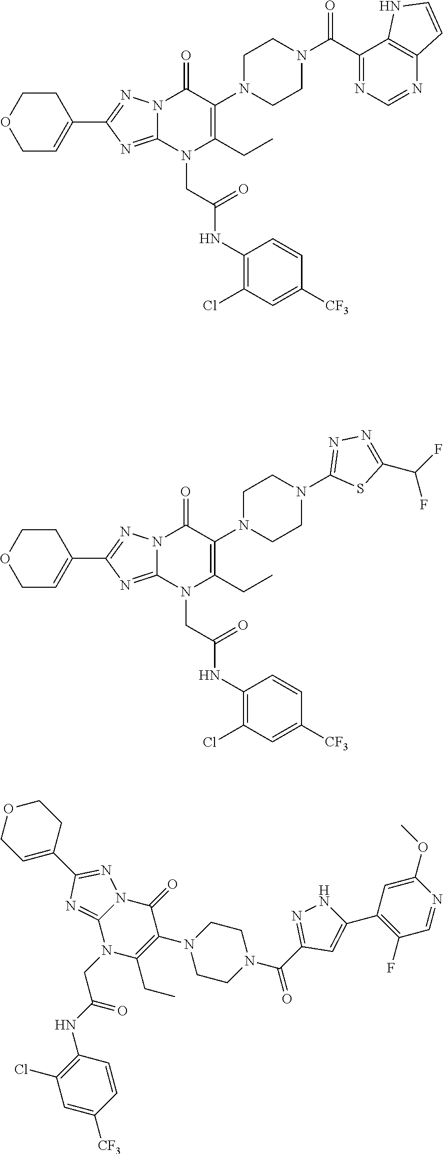 [SMILES: CCc1c(N2CCN(C(=O)c3ncnc4cc[nH]c34)CC2)c(=O)n2nc(C3=CCOCC3)nc2n1CC(=O)Nc1ccc(C(F)(F)F)cc1Cl ; CCc1c(N2CCN(c3nnc(C(F)F)s3)CC2)c(=O)n2nc(C3=CCOCC3)nc2n1CC(=O)Nc1ccc(C(F)(F)F)cc1Cl ; CCc1c(N2CCN(C(=O)c3cc(-c4cc(OC)ncc4F)[nH]n3)CC2)c(=O)n2nc(C3=CCOCC3)nc2n1CC(=O)Nc1ccc(C(F)(F)F)cc1Cl] 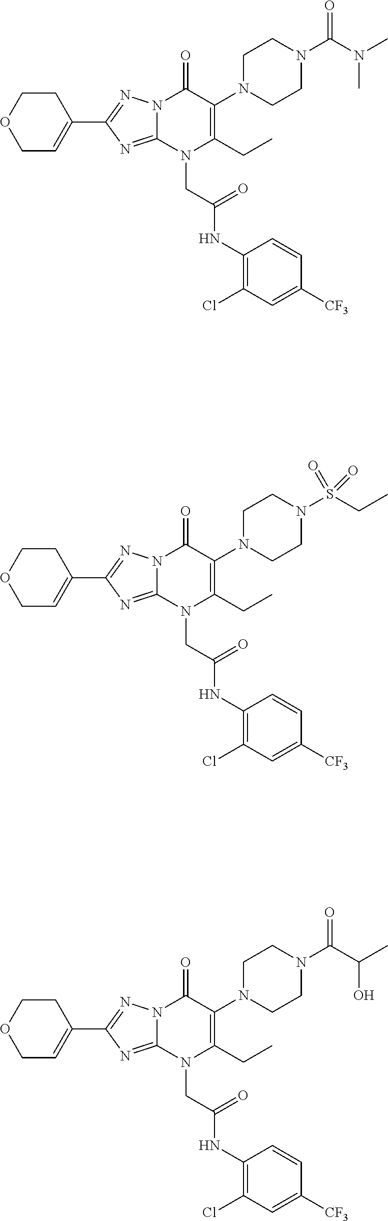 [SMILES: CCc1c(N2CCN(C(=O)N(C)C)CC2)c(=O)n2nc(C3=CCOCC3)nc2n1CC(=O)Nc1ccc(C(F)(F)F)cc1Cl ; CCc1c(N2CCN(S(=O)(=O)CC)CC2)c(=O)n2nc(C3=CCOCC3)nc2n1CC(=O)Nc1ccc(C(F)(F)F)cc1Cl ; CCc1c(N2CCN(C(=O)C(C)O)CC2)c(=O)n2nc(C3=CCOCC3)nc2n1CC(=O)Nc1ccc(C(F)(F)F)cc1Cl] 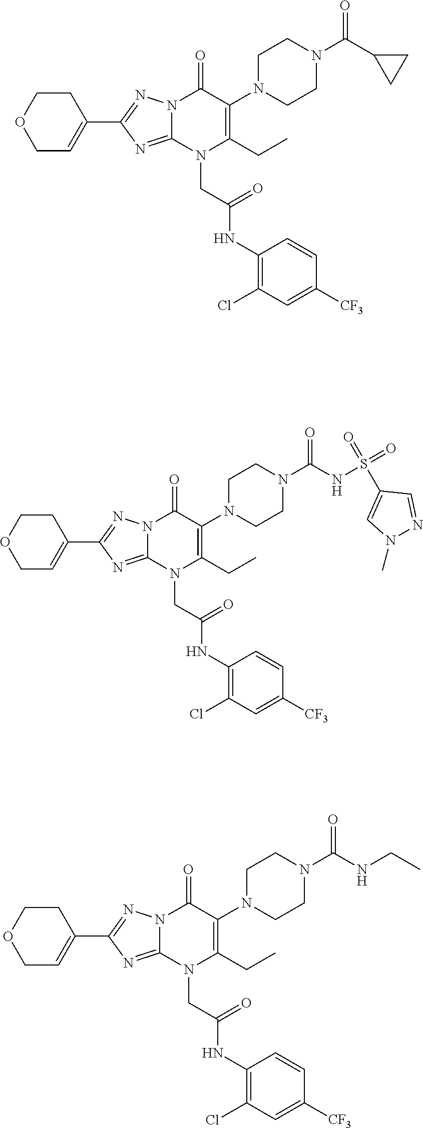 [SMILES: CCc1c(N2CCN(C(=O)C3CC3)CC2)c(=O)n2nc(C3=CCOCC3)nc2n1CC(=O)Nc1ccc(C(F)(F)F)cc1Cl ; CCc1c(N2CCN(C(=O)NS(=O)(=O)c3cnn(C)c3)CC2)c(=O)n2nc(C3=CCOCC3)nc2n1CC(=O)Nc1ccc(C(F)(F)F)cc1Cl ; CCNC(=O)N1CCN(c2c(CC)n(CC(=O)Nc3ccc(C(F)(F)F)cc3Cl)c3nc(C4=CCOCC4)nn3c2=O)CC1] 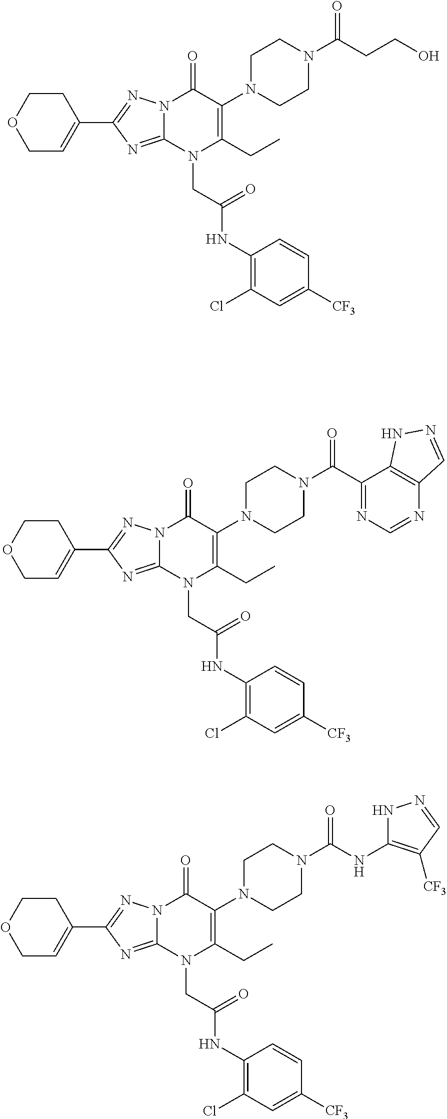 [SMILES: CCc1c(N2CCN(C(=O)CCO)CC2)c(=O)n2nc(C3=CCOCC3)nc2n1CC(=O)Nc1ccc(C(F)(F)F)cc1Cl ; CCc1c(N2CCN(C(=O)c3ncnc4cn[nH]c34)CC2)c(=O)n2nc(C3=CCOCC3)nc2n1CC(=O)Nc1ccc(C(F)(F)F)cc1Cl ; CCc1c(N2CCN(C(=O)Nc3[nH]ncc3C(F)(F)F)CC2)c(=O)n2nc(C3=CCOCC3)nc2n1CC(=O)Nc1ccc(C(F)(F)F)cc1Cl] 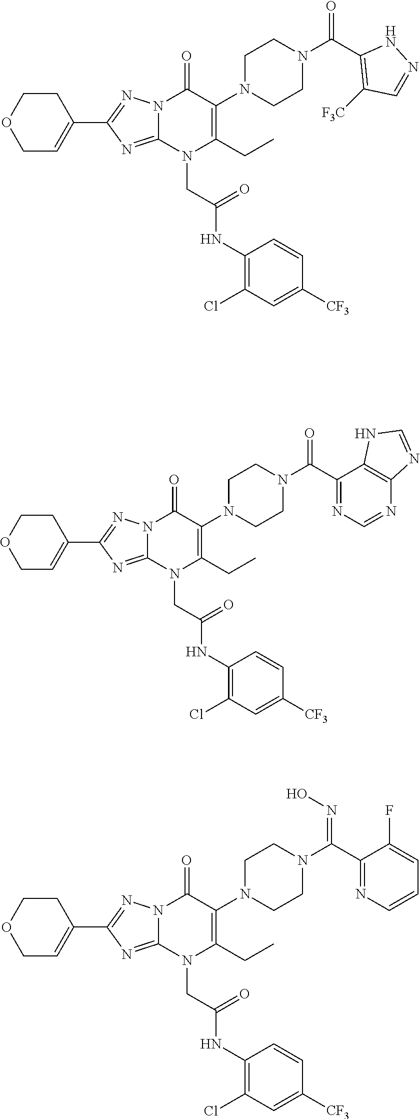 [SMILES: CCc1c(N2CCN(C(=O)c3[nH]ncc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3ncnc4nc[nH]c34)CC2)c(=O)n2nc(C3=CCOCC3)nc2n1CC(=O)Nc1ccc(C(F)(F)F)cc1Cl ; CCc1c(N2CCN(/C(=N\O)c3ncccc3F)CC2)c(=O)n2nc(C3=CCOCC3)nc2n1CC(=O)Nc1ccc(C(F)(F)F)cc1Cl] 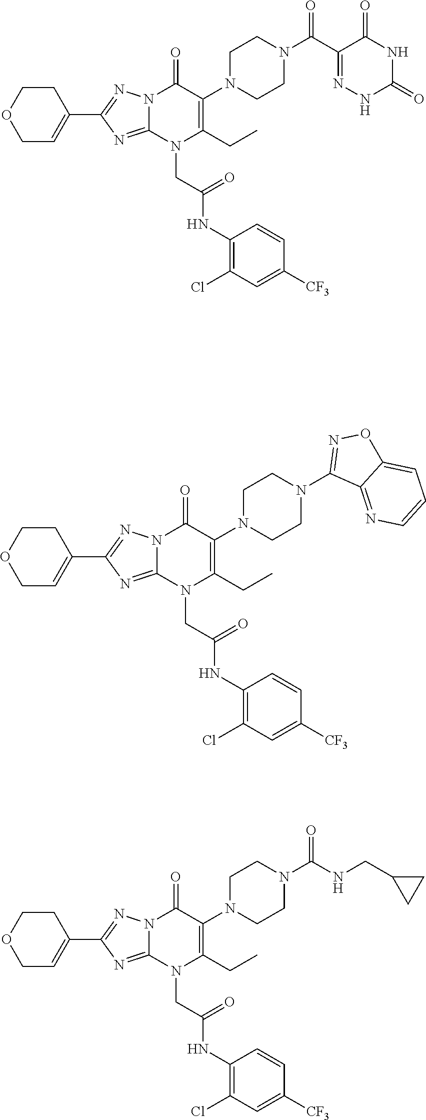 [SMILES: CCc1c(N2CCN(C(=O)c3n[nH]c(=O)[nH]c3=O)CC2)c(=O)n2nc(C3=CCOCC3)nc2n1CC(=O)Nc1ccc(C(F)(F)F)cc1Cl ; CCc1c(N2CCN(c3noc4cccnc34)CC2)c(=O)n2nc(C3=CCOCC3)nc2n1CC(=O)Nc1ccc(C(F)(F)F)cc1Cl ; CCc1c(N2CCN(C(=O)NCC3CC3)CC2)c(=O)n2nc(C3=CCOCC3)nc2n1CC(=O)Nc1ccc(C(F)(F)F)cc1Cl] 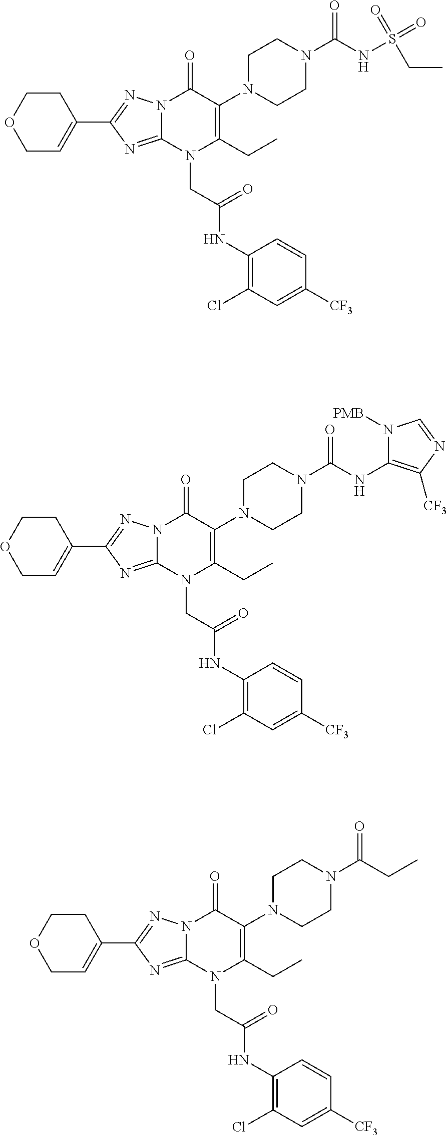 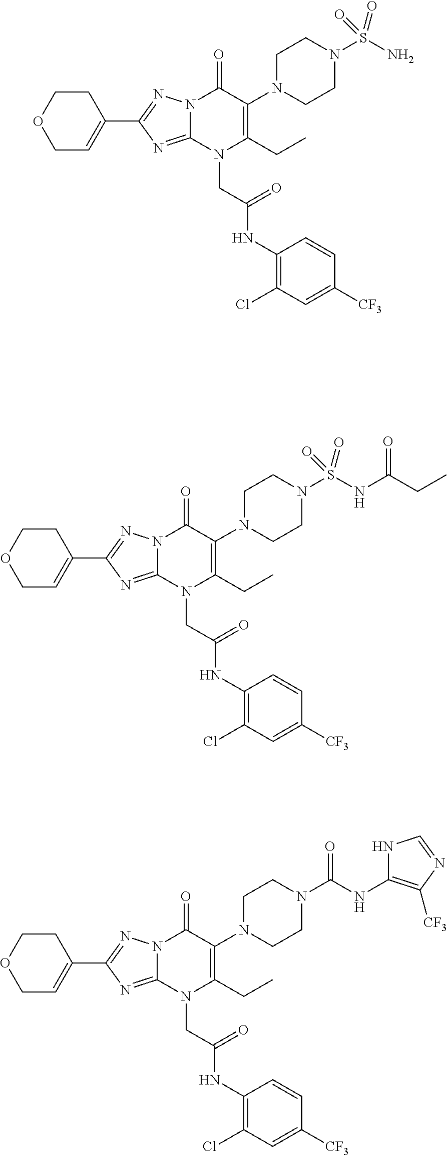 [SMILES: CCc1c(N2CCN(S(N)(=O)=O)CC2)c(=O)n2nc(C3=CCOCC3)nc2n1CC(=O)Nc1ccc(C(F)(F)F)cc1Cl ; CCC(=O)NS(=O)(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)Nc3[nH]cnc3C(F)(F)F)CC2)c(=O)n2nc(C3=CCOCC3)nc2n1CC(=O)Nc1ccc(C(F)(F)F)cc1Cl] 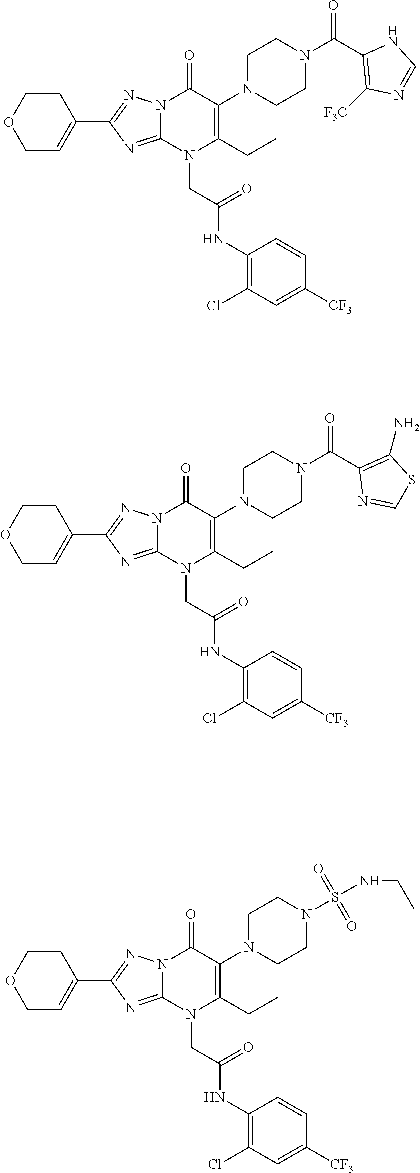 [SMILES: CCc1c(N2CCN(C(=O)c3[nH]cnc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3ncsc3N)CC2)c(=O)n2nc(C3=CCOCC3)nc2n1CC(=O)Nc1ccc(C(F)(F)F)cc1Cl ; CCNS(=O)(=O)N1CCN(c2c(CC)n(CC(=O)Nc3ccc(C(F)(F)F)cc3Cl)c3nc(C4=CCOCC4)nn3c2=O)CC1] 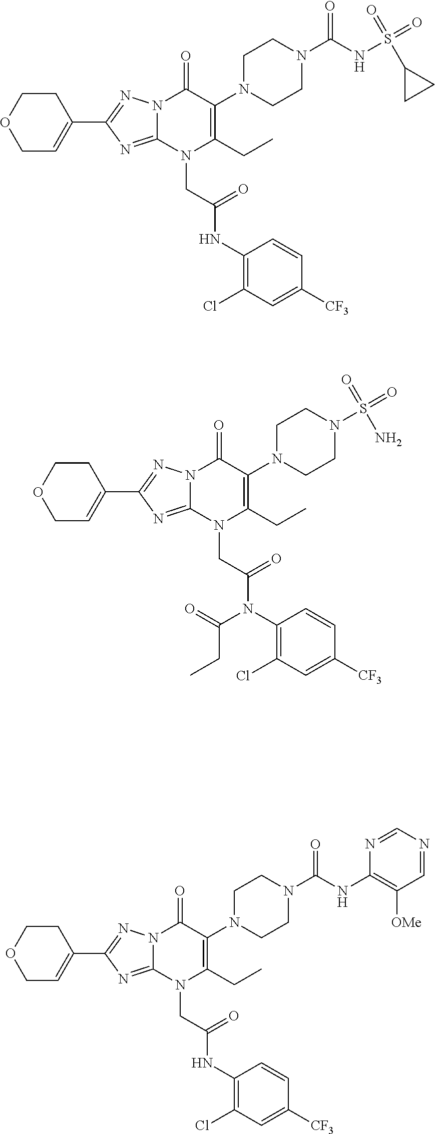 [SMILES: CCc1c(N2CCN(C(=O)NS(=O)(=O)C3CC3)CC2)c(=O)n2nc(C3=CCOCC3)nc2n1CC(=O)Nc1ccc(C(F)(F)F)cc1Cl ; CCC(=O)N(C(=O)Cn1c(CC)c(N2CCN(S(N)(=O)=O)CC2)c(=O)n2nc(C3=CCOCC3)nc12)c1ccc(C(F)(F)F)cc1Cl ; CCc1c(N2CCN(C(=O)Nc3ncncc3OC)CC2)c(=O)n2nc(C3=CCOCC3)nc2n1CC(=O)Nc1ccc(C(F)(F)F)cc1Cl] 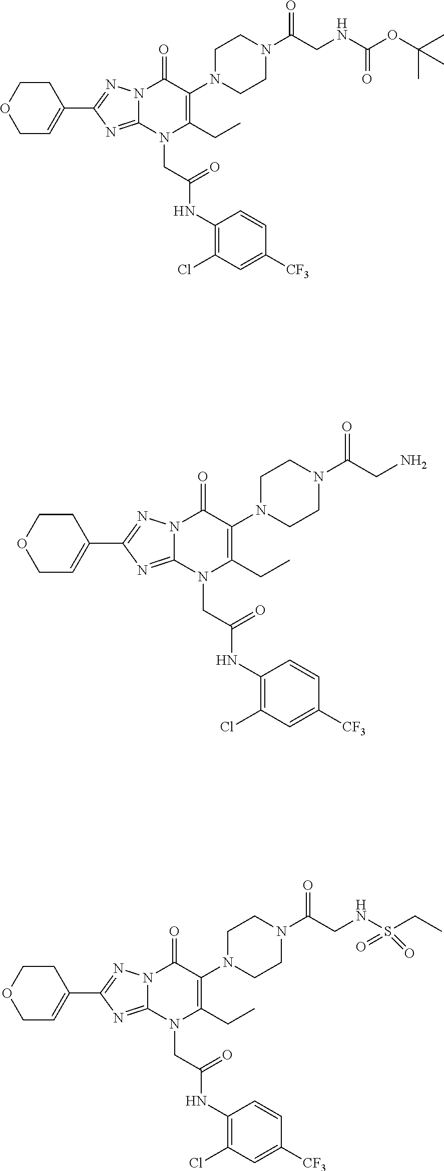 [SMILES: CCc1c(N2CCN(C(=O)CNC(=O)OC(C)(C)C)CC2)c(=O)n2nc(C3=CCOCC3)nc2n1CC(=O)Nc1ccc(C(F)(F)F)cc1Cl ; CCc1c(N2CCN(C(=O)CN)CC2)c(=O)n2nc(C3=CCOCC3)nc2n1CC(=O)Nc1ccc(C(F)(F)F)cc1Cl ; CCc1c(N2CCN(C(=O)CNS(=O)(=O)CC)CC2)c(=O)n2nc(C3=CCOCC3)nc2n1CC(=O)Nc1ccc(C(F)(F)F)cc1Cl] 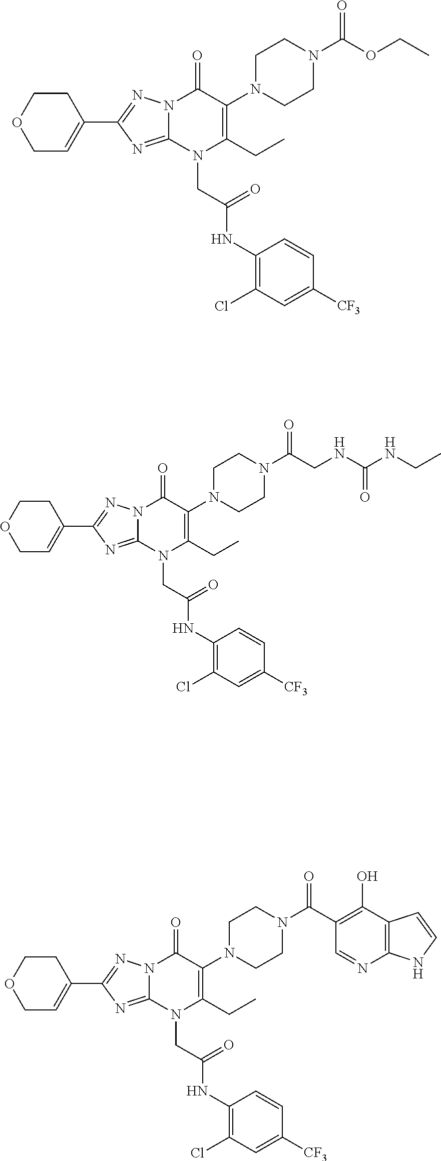 [SMILES: CCOC(=O)N1CCN(c2c(CC)n(CC(=O)Nc3ccc(C(F)(F)F)cc3Cl)c3nc(C4=CCOCC4)nn3c2=O)CC1 ; CCNC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)c3cnc4[nH]ccc4c3O)CC2)c(=O)n2nc(C3=CCOCC3)nc2n1CC(=O)Nc1ccc(C(F)(F)F)cc1Cl] 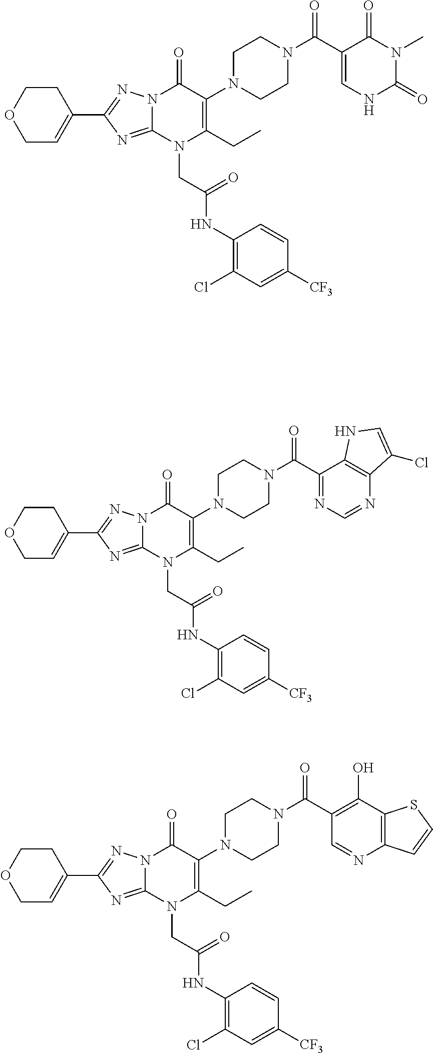 [SMILES: CCc1c(N2CCN(C(=O)c3c[nH]c(=O)n(C)c3=O)CC2)c(=O)n2nc(C3=CCOCC3)nc2n1CC(=O)Nc1ccc(C(F)(F)F)cc1Cl ; CCc1c(N2CCN(C(=O)c3ncnc4c(Cl)c[nH]c34)CC2)c(=O)n2nc(C3=CCOCC3)nc2n1CC(=O)Nc1ccc(C(F)(F)F)cc1Cl ; CCc1c(N2CCN(C(=O)c3cnc4ccsc4c3O)CC2)c(=O)n2nc(C3=CCOCC3)nc2n1CC(=O)Nc1ccc(C(F)(F)F)cc1Cl] 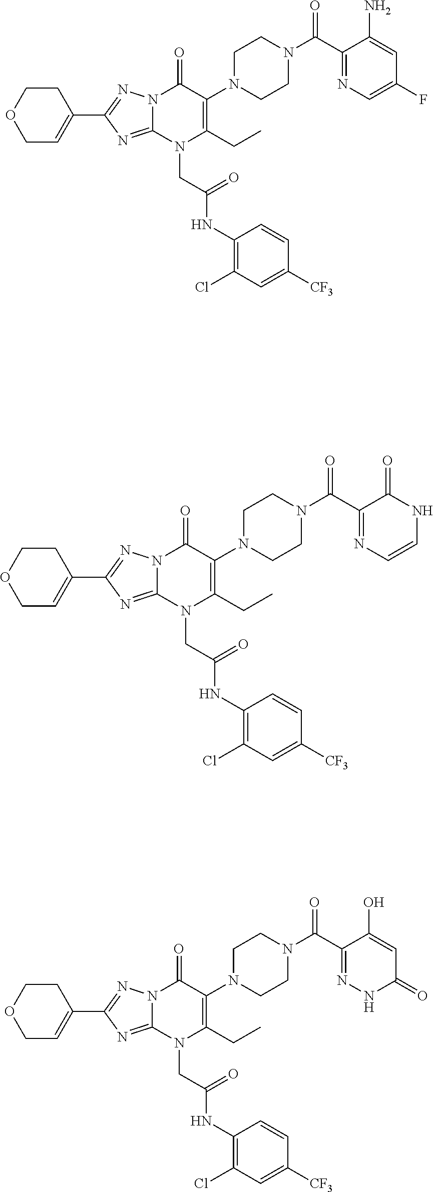 [SMILES: CCc1c(N2CCN(C(=O)c3ncc(F)cc3N)CC2)c(=O)n2nc(C3=CCOCC3)nc2n1CC(=O)Nc1ccc(C(F)(F)F)cc1Cl ; CCc1c(N2CCN(C(=O)c3ncc[nH]c3=O)CC2)c(=O)n2nc(C3=CCOCC3)nc2n1CC(=O)Nc1ccc(C(F)(F)F)cc1Cl ; CCc1c(N2CCN(C(=O)c3n[nH]c(=O)cc3O)CC2)c(=O)n2nc(C3=CCOCC3)nc2n1CC(=O)Nc1ccc(C(F)(F)F)cc1Cl] 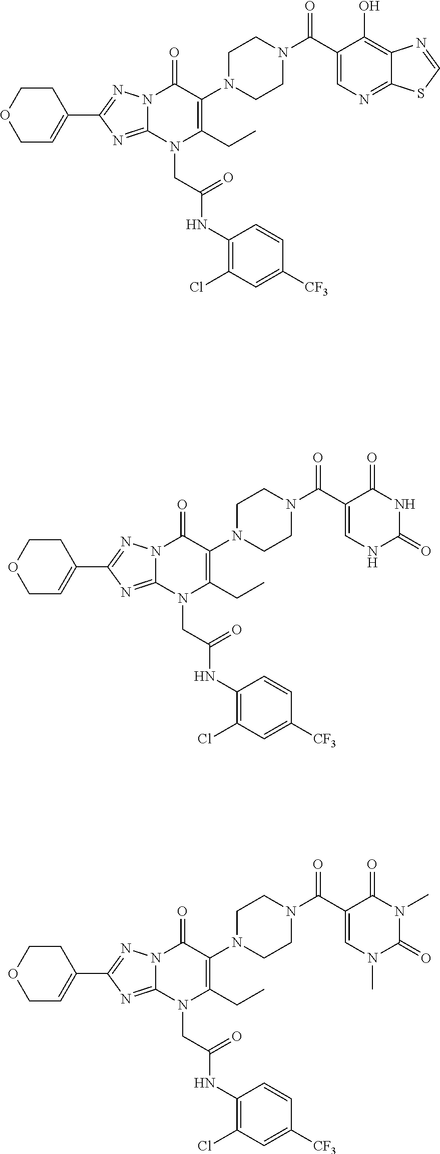 [SMILES: CCc1c(N2CCN(C(=O)c3cnc4scnc4c3O)CC2)c(=O)n2nc(C3=CCOCC3)nc2n1CC(=O)Nc1ccc(C(F)(F)F)cc1Cl ; CCc1c(N2CCN(C(=O)c3c[nH]c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cn(C)c(=O)n(C)c3=O)CC2)c(=O)n2nc(C3=CCOCC3)nc2n1CC(=O)Nc1ccc(C(F)(F)F)cc1Cl] 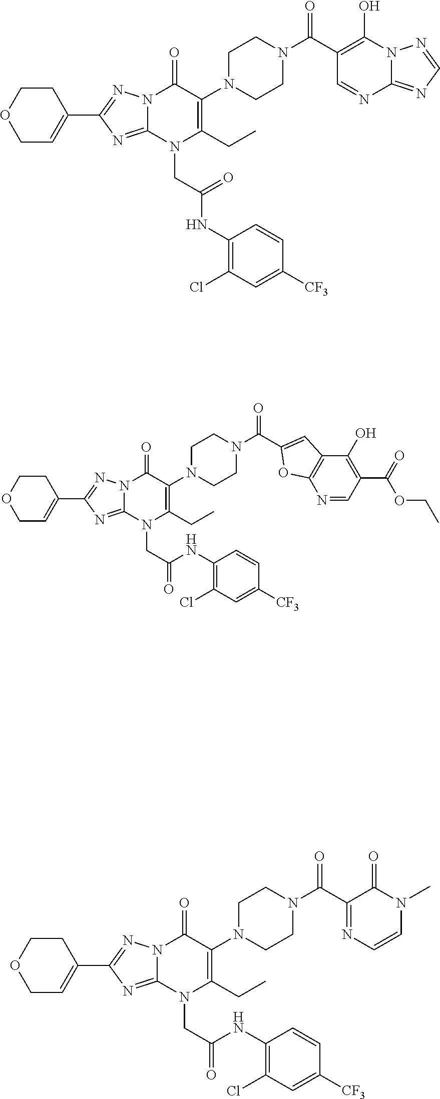 [SMILES: CCc1c(N2CCN(C(=O)c3cnc4ncnn4c3O)CC2)c(=O)n2nc(C3=CCOCC3)nc2n1CC(=O)Nc1ccc(C(F)(F)F)cc1Cl ; CCOC(=O)c1cnc2oc(C(=O)N3CCN(c4c(CC)n(CC(=O)Nc5ccc(C(F)(F)F)cc5Cl)c5nc(C6=CCOCC6)nn5c4=O)CC3)cc2c1O ; CCc1c(N2CCN(C(=O)c3nccn(C)c3=O)CC2)c(=O)n2nc(C3=CCOCC3)nc2n1CC(=O)Nc1ccc(C(F)(F)F)cc1Cl] 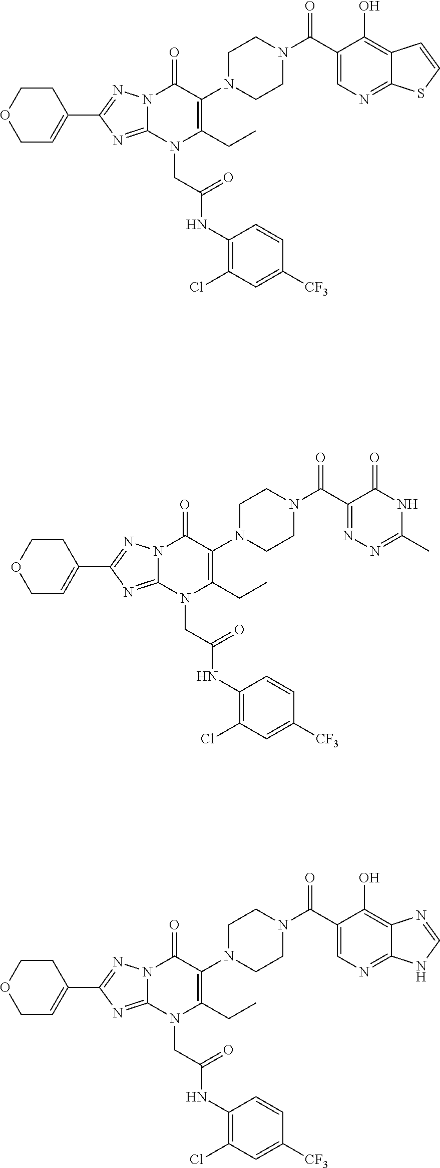 [SMILES: CCc1c(N2CCN(C(=O)c3cnc4sccc4c3O)CC2)c(=O)n2nc(C3=CCOCC3)nc2n1CC(=O)Nc1ccc(C(F)(F)F)cc1Cl ; CCc1c(N2CCN(C(=O)c3nnc(C)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cnc4[nH]cnc4c3O)CC2)c(=O)n2nc(C3=CCOCC3)nc2n1CC(=O)Nc1ccc(C(F)(F)F)cc1Cl] 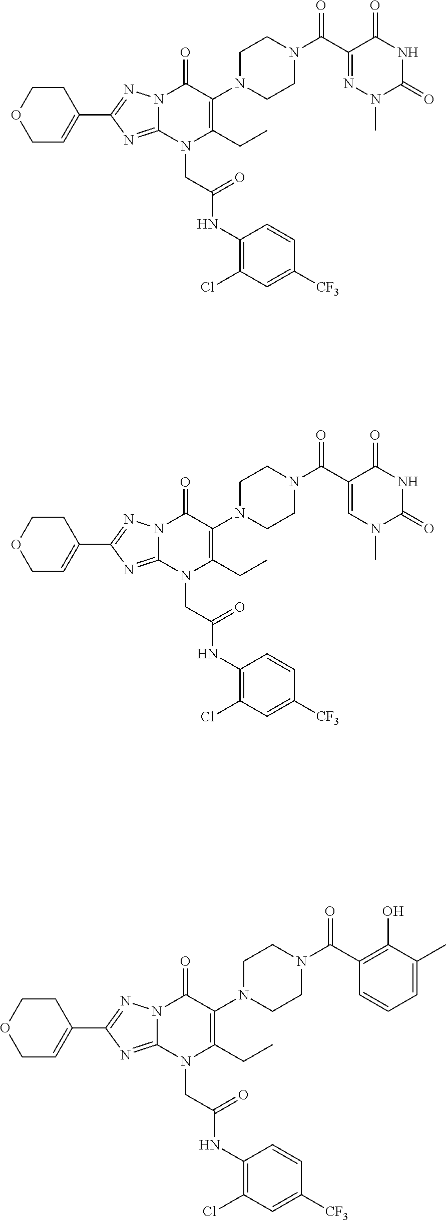 [SMILES: CCc1c(N2CCN(C(=O)c3nn(C)c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cn(C)c(=O)[nH]c3=O)CC2)c(=O)n2nc(C3=CCOCC3)nc2n1CC(=O)Nc1ccc(C(F)(F)F)cc1Cl ; CCc1c(N2CCN(C(=O)c3cccc(C)c3O)CC2)c(=O)n2nc(C3=CCOCC3)nc2n1CC(=O)Nc1ccc(C(F)(F)F)cc1Cl] 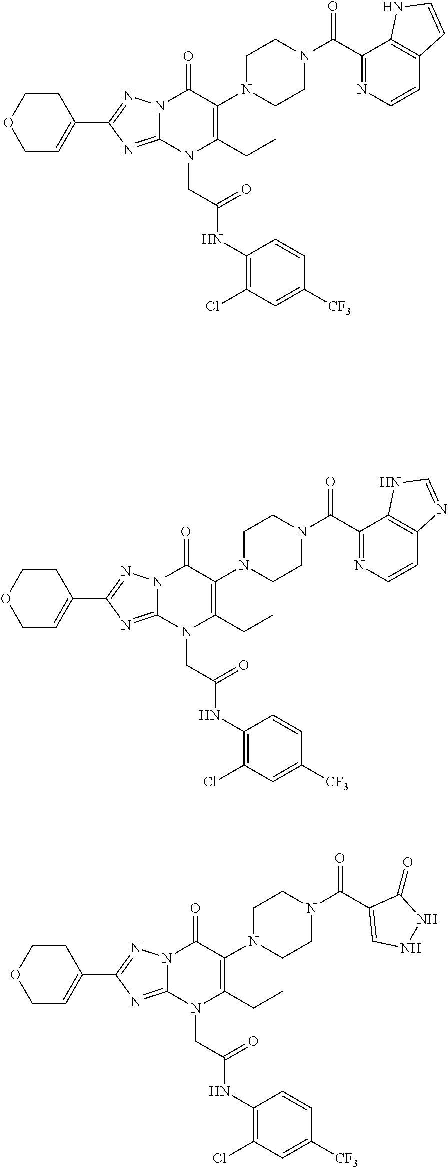 [SMILES: CCc1c(N2CCN(C(=O)c3nccc4cc[nH]c34)CC2)c(=O)n2nc(C3=CCOCC3)nc2n1CC(=O)Nc1ccc(C(F)(F)F)cc1Cl ; CCc1c(N2CCN(C(=O)c3nccc4nc[nH]c34)CC2)c(=O)n2nc(C3=CCOCC3)nc2n1CC(=O)Nc1ccc(C(F)(F)F)cc1Cl ; CCc1c(N2CCN(C(=O)c3c[nH][nH]c3=O)CC2)c(=O)n2nc(C3=CCOCC3)nc2n1CC(=O)Nc1ccc(C(F)(F)F)cc1Cl] 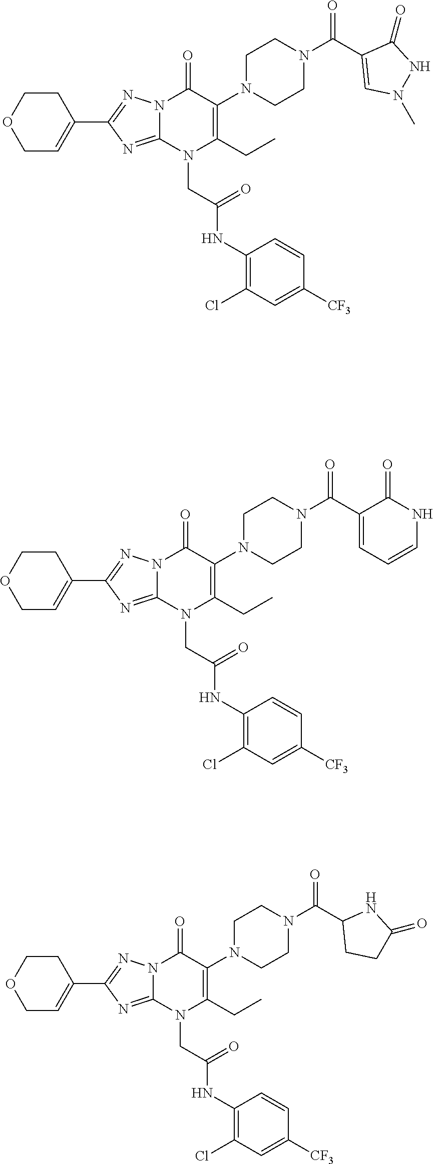 [SMILES: CCc1c(N2CCN(C(=O)c3cn(C)[nH]c3=O)CC2)c(=O)n2nc(C3=CCOCC3)nc2n1CC(=O)Nc1ccc(C(F)(F)F)cc1Cl ; CCc1c(N2CCN(C(=O)c3ccc[nH]c3=O)CC2)c(=O)n2nc(C3=CCOCC3)nc2n1CC(=O)Nc1ccc(C(F)(F)F)cc1Cl ; CCc1c(N2CCN(C(=O)C3CCC(=O)N3)CC2)c(=O)n2nc(C3=CCOCC3)nc2n1CC(=O)Nc1ccc(C(F)(F)F)cc1Cl] 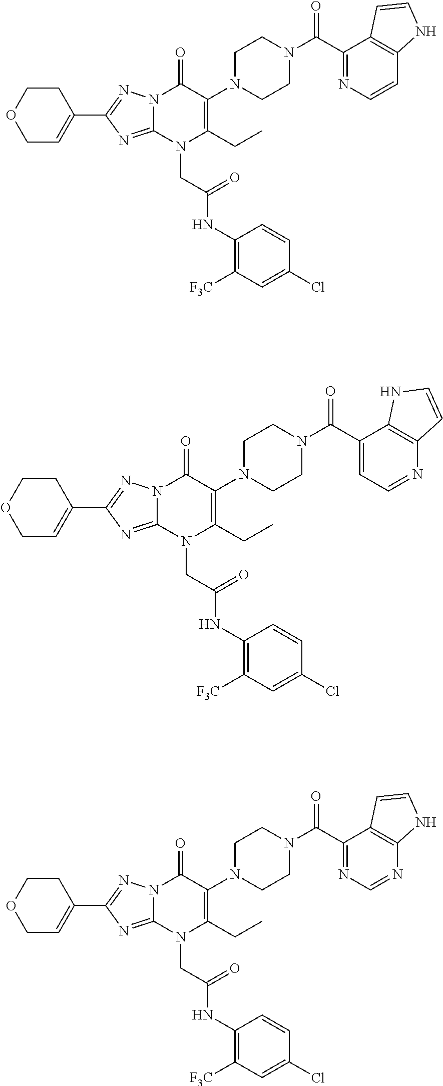 [SMILES: CCc1c(N2CCN(C(=O)c3nccc4[nH]ccc34)CC2)c(=O)n2nc(C3=CCOCC3)nc2n1CC(=O)Nc1ccc(Cl)cc1C(F)(F)F ; CCc1c(N2CCN(C(=O)c3ccnc4cc[nH]c34)CC2)c(=O)n2nc(C3=CCOCC3)nc2n1CC(=O)Nc1ccc(Cl)cc1C(F)(F)F ; CCc1c(N2CCN(C(=O)c3ncnc4[nH]ccc34)CC2)c(=O)n2nc(C3=CCOCC3)nc2n1CC(=O)Nc1ccc(Cl)cc1C(F)(F)F] 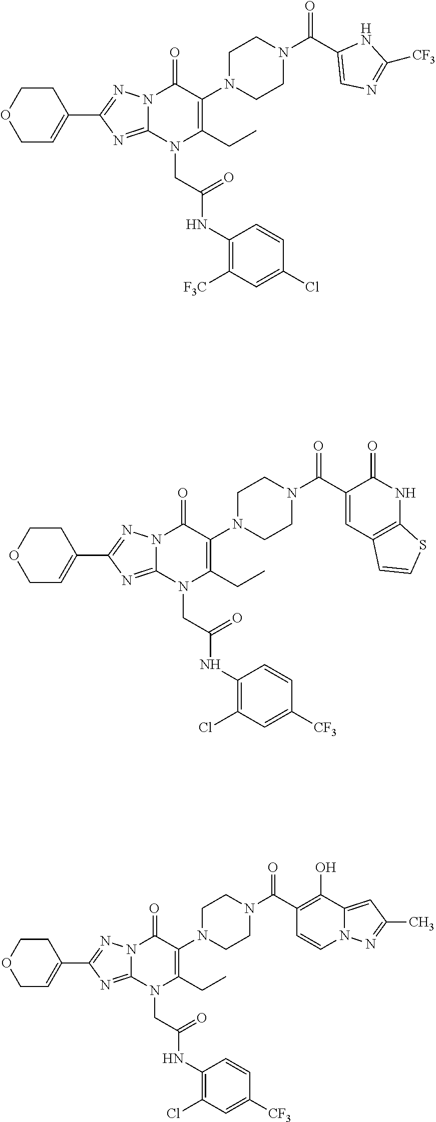 [SMILES: CCc1c(N2CCN(C(=O)c3cnc(C(F)(F)F)[nH]3)CC2)c(=O)n2nc(C3=CCOCC3)nc2n1CC(=O)Nc1ccc(Cl)cc1C(F)(F)F ; CCc1c(N2CCN(C(=O)c3cc4ccsc4[nH]c3=O)CC2)c(=O)n2nc(C3=CCOCC3)nc2n1CC(=O)Nc1ccc(C(F)(F)F)cc1Cl ; CCc1c(N2CCN(C(=O)c3ccn4nc(C)cc4c3O)CC2)c(=O)n2nc(C3=CCOCC3)nc2n1CC(=O)Nc1ccc(C(F)(F)F)cc1Cl] 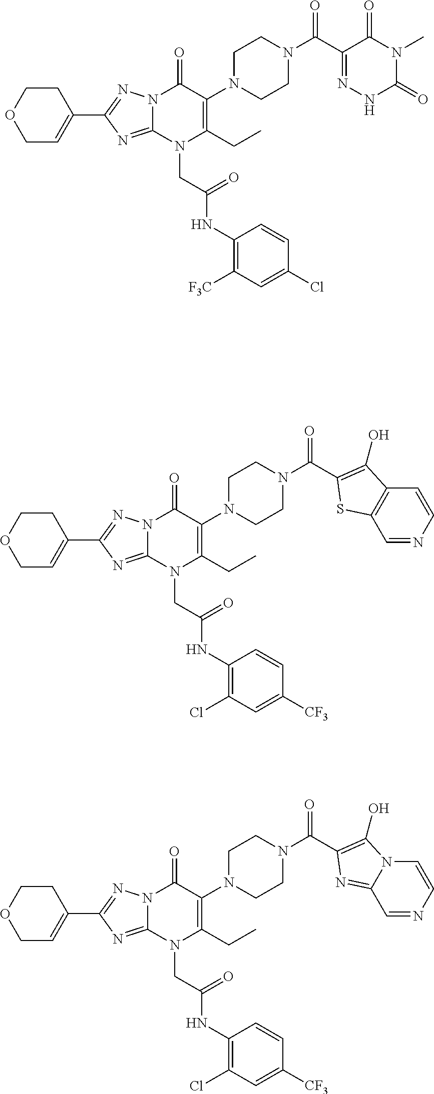 [SMILES: CCc1c(N2CCN(C(=O)c3n[nH]c(=O)n(C)c3=O)CC2)c(=O)n2nc(C3=CCOCC3)nc2n1CC(=O)Nc1ccc(Cl)cc1C(F)(F)F ; CCc1c(N2CCN(C(=O)c3sc4cnccc4c3O)CC2)c(=O)n2nc(C3=CCOCC3)nc2n1CC(=O)Nc1ccc(C(F)(F)F)cc1Cl ; CCc1c(N2CCN(C(=O)c3nc4cnccn4c3O)CC2)c(=O)n2nc(C3=CCOCC3)nc2n1CC(=O)Nc1ccc(C(F)(F)F)cc1Cl] 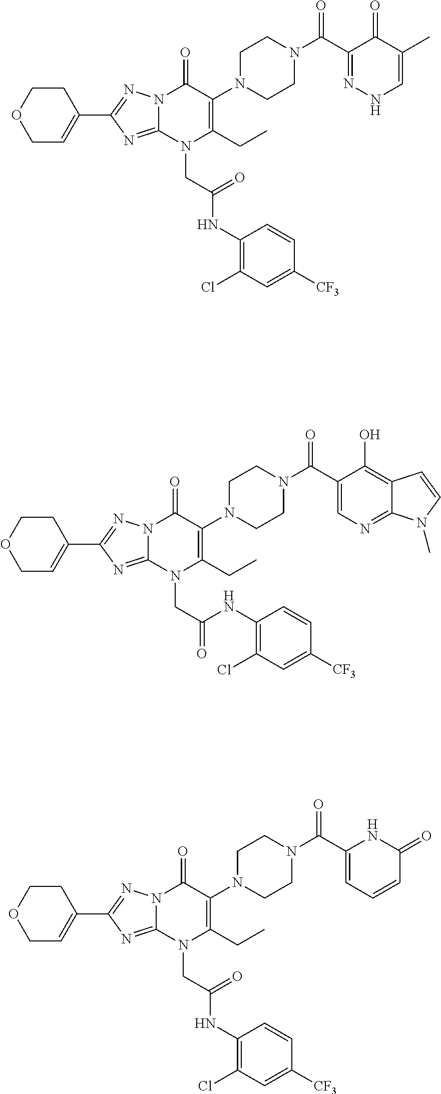 [SMILES: CCc1c(N2CCN(C(=O)c3n[nH]cc(C)c3=O)CC2)c(=O)n2nc(C3=CCOCC3)nc2n1CC(=O)Nc1ccc(C(F)(F)F)cc1Cl ; CCc1c(N2CCN(C(=O)c3cnc4c(ccn4C)c3O)CC2)c(=O)n2nc(C3=CCOCC3)nc2n1CC(=O)Nc1ccc(C(F)(F)F)cc1Cl ; CCc1c(N2CCN(C(=O)c3cccc(=O)[nH]3)CC2)c(=O)n2nc(C3=CCOCC3)nc2n1CC(=O)Nc1ccc(C(F)(F)F)cc1Cl] 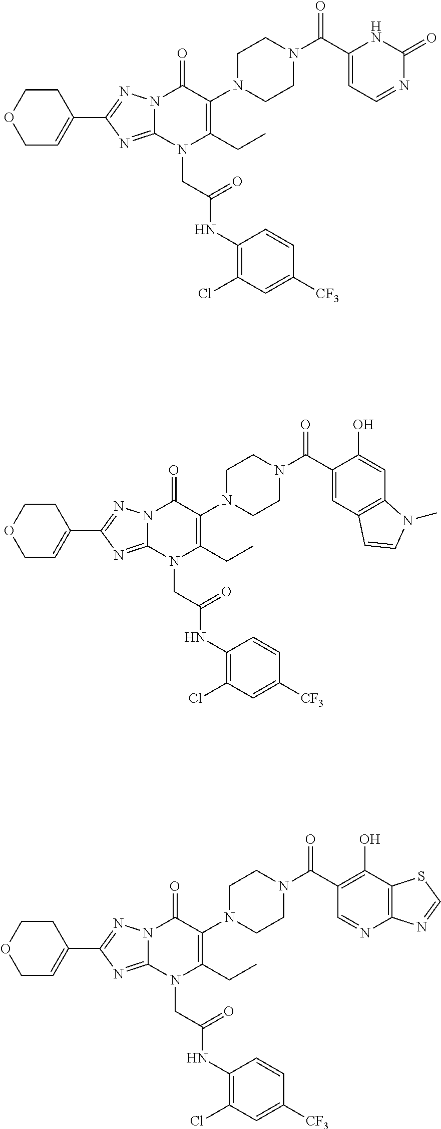 [SMILES: CCc1c(N2CCN(C(=O)c3ccnc(=O)[nH]3)CC2)c(=O)n2nc(C3=CCOCC3)nc2n1CC(=O)Nc1ccc(C(F)(F)F)cc1Cl ; CCc1c(N2CCN(C(=O)c3cc4ccn(C)c4cc3O)CC2)c(=O)n2nc(C3=CCOCC3)nc2n1CC(=O)Nc1ccc(C(F)(F)F)cc1Cl ; CCc1c(N2CCN(C(=O)c3cnc4ncsc4c3O)CC2)c(=O)n2nc(C3=CCOCC3)nc2n1CC(=O)Nc1ccc(C(F)(F)F)cc1Cl] 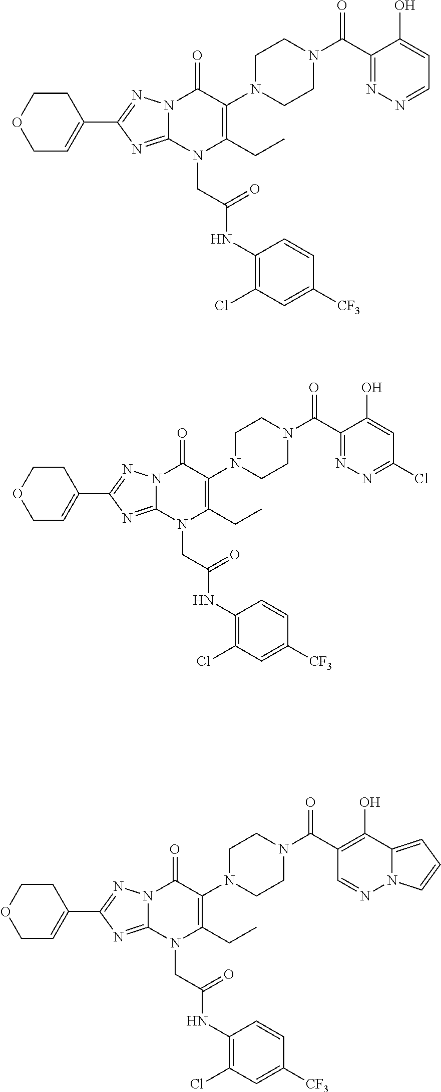 [SMILES: CCc1c(N2CCN(C(=O)c3nnccc3O)CC2)c(=O)n2nc(C3=CCOCC3)nc2n1CC(=O)Nc1ccc(C(F)(F)F)cc1Cl ; CCc1c(N2CCN(C(=O)c3nnc(Cl)cc3O)CC2)c(=O)n2nc(C3=CCOCC3)nc2n1CC(=O)Nc1ccc(C(F)(F)F)cc1Cl ; CCc1c(N2CCN(C(=O)c3cnn4cccc4c3O)CC2)c(=O)n2nc(C3=CCOCC3)nc2n1CC(=O)Nc1ccc(C(F)(F)F)cc1Cl] 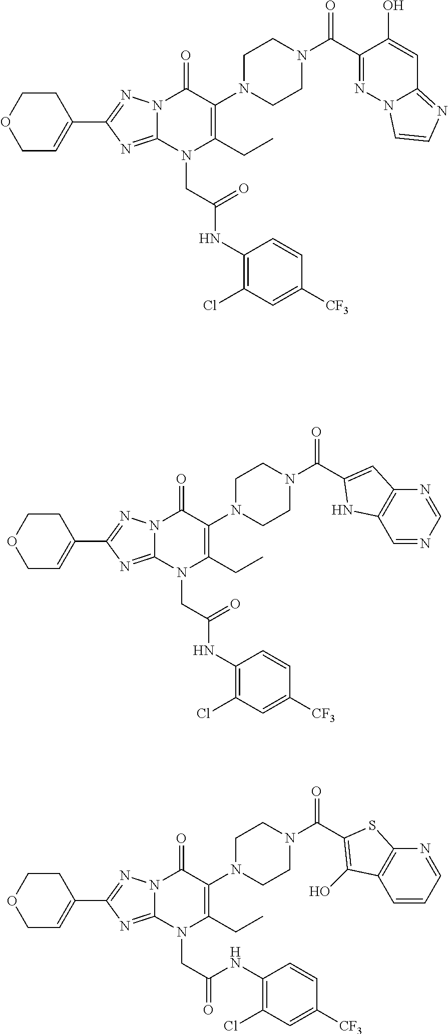 [SMILES: CCc1c(N2CCN(C(=O)c3nn4ccnc4cc3O)CC2)c(=O)n2nc(C3=CCOCC3)nc2n1CC(=O)Nc1ccc(C(F)(F)F)cc1Cl ; CCc1c(N2CCN(C(=O)c3cc4ncncc4[nH]3)CC2)c(=O)n2nc(C3=CCOCC3)nc2n1CC(=O)Nc1ccc(C(F)(F)F)cc1Cl ; CCc1c(N2CCN(C(=O)c3sc4ncccc4c3O)CC2)c(=O)n2nc(C3=CCOCC3)nc2n1CC(=O)Nc1ccc(C(F)(F)F)cc1Cl] 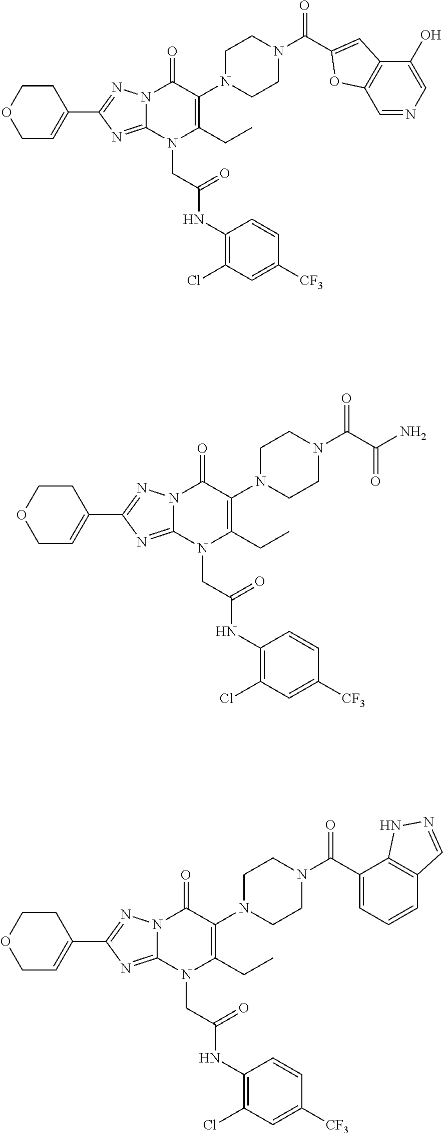 [SMILES: CCc1c(N2CCN(C(=O)c3cc4c(O)cncc4o3)CC2)c(=O)n2nc(C3=CCOCC3)nc2n1CC(=O)Nc1ccc(C(F)(F)F)cc1Cl ; CCc1c(N2CCN(C(=O)C(N)=O)CC2)c(=O)n2nc(C3=CCOCC3)nc2n1CC(=O)Nc1ccc(C(F)(F)F)cc1Cl ; CCc1c(N2CCN(C(=O)c3cccc4cn[nH]c34)CC2)c(=O)n2nc(C3=CCOCC3)nc2n1CC(=O)Nc1ccc(C(F)(F)F)cc1Cl] 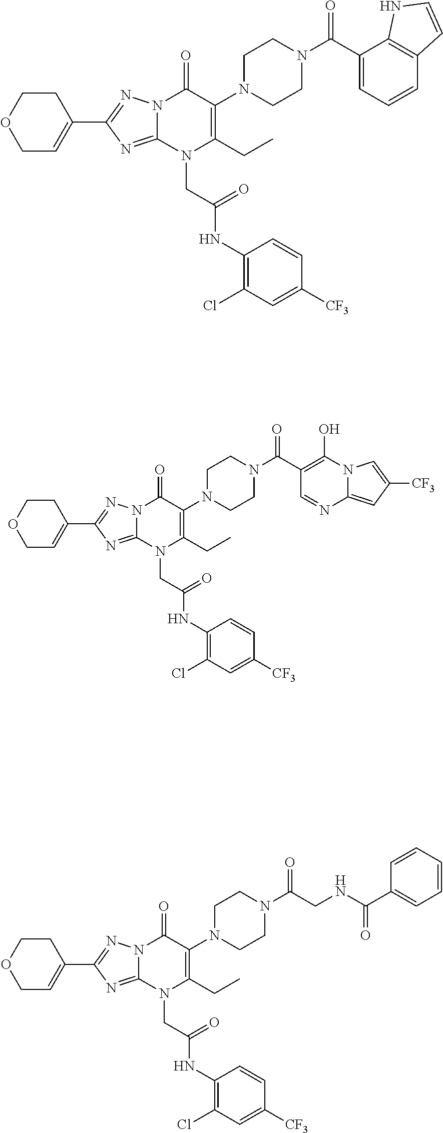 [SMILES: CCc1c(N2CCN(C(=O)c3cccc4cc[nH]c34)CC2)c(=O)n2nc(C3=CCOCC3)nc2n1CC(=O)Nc1ccc(C(F)(F)F)cc1Cl ; CCc1c(N2CCN(C(=O)c3cnc4cc(C(F)(F)F)cn4c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3)CC2)c(=O)n2nc(C3=CCOCC3)nc2n1CC(=O)Nc1ccc(C(F)(F)F)cc1Cl] 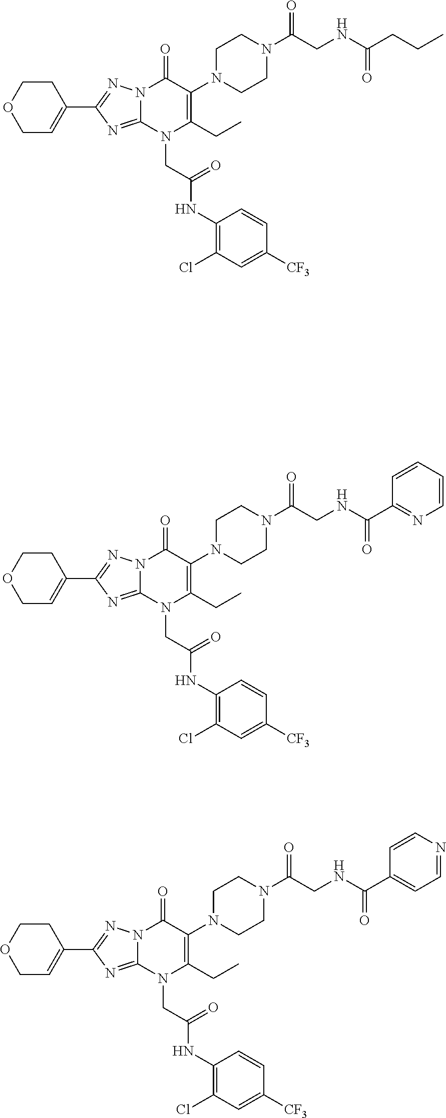 [SMILES: CCCC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)CNC(=O)c3ccccn3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccncc3)CC2)c(=O)n2nc(C3=CCOCC3)nc2n1CC(=O)Nc1ccc(C(F)(F)F)cc1Cl] 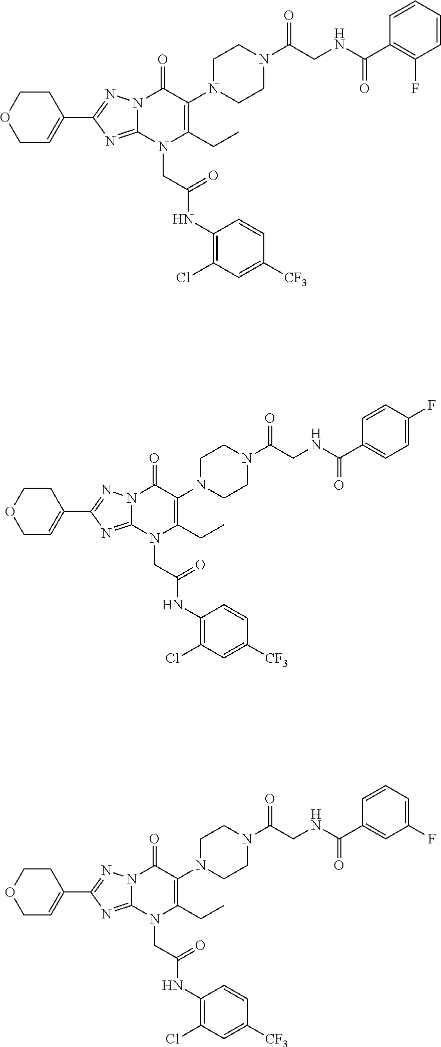 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)cc3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(F)c3)CC2)c(=O)n2nc(C3=CCOCC3)nc2n1CC(=O)Nc1ccc(C(F)(F)F)cc1Cl] 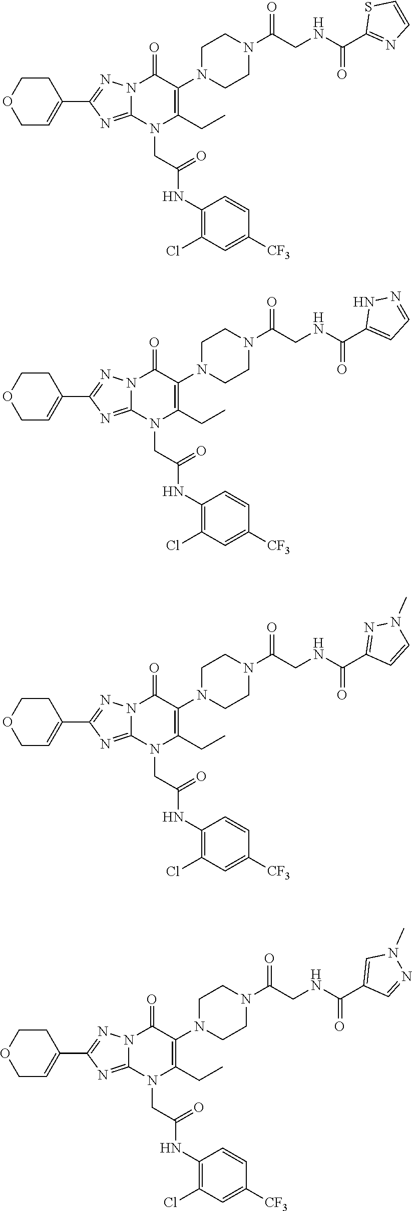 [SMILES: CCc1c(N2CCN(C(=O)CNC(=O)c3nccs3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccn[nH]3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccn(C)n3)CC2)c(=O)n2nc(C3=CCOCC3)nc2n1CC(=O)Nc1ccc(C(F)(F)F)cc1Cl ; CCc1c(N2CCN(C(=O)CNC(=O)c3cnn(C)c3)CC2)c(=O)n2nc(C3=CCOCC3)nc2n1CC(=O)Nc1ccc(C(F)(F)F)cc1Cl] 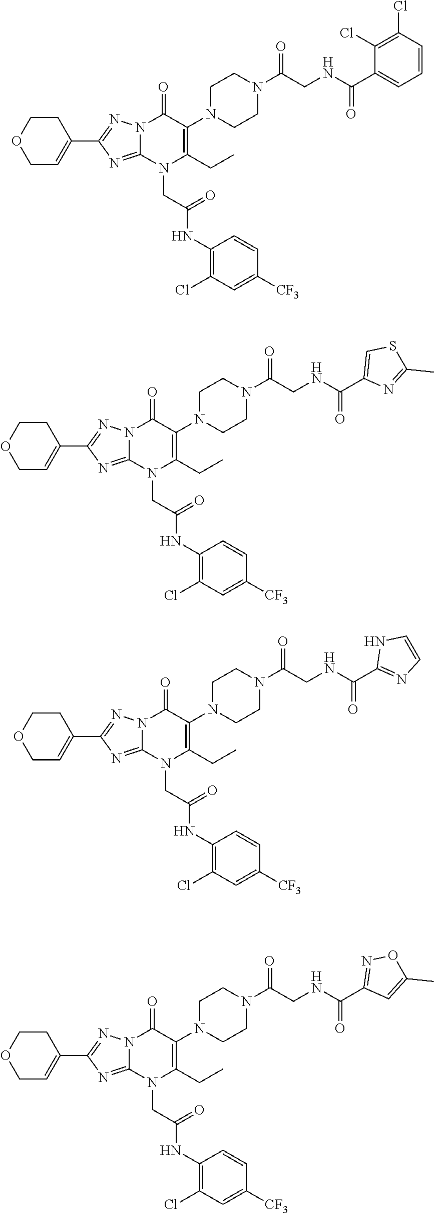 [SMILES: CCc1c(N2CCN(C(=O)CNC(=O)c3cccc(Cl)c3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3csc(C)n3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncc[nH]3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C)on3)CC2)c(=O)n2nc(C3=CCOCC3)nc2n1CC(=O)Nc1ccc(C(F)(F)F)cc1Cl] 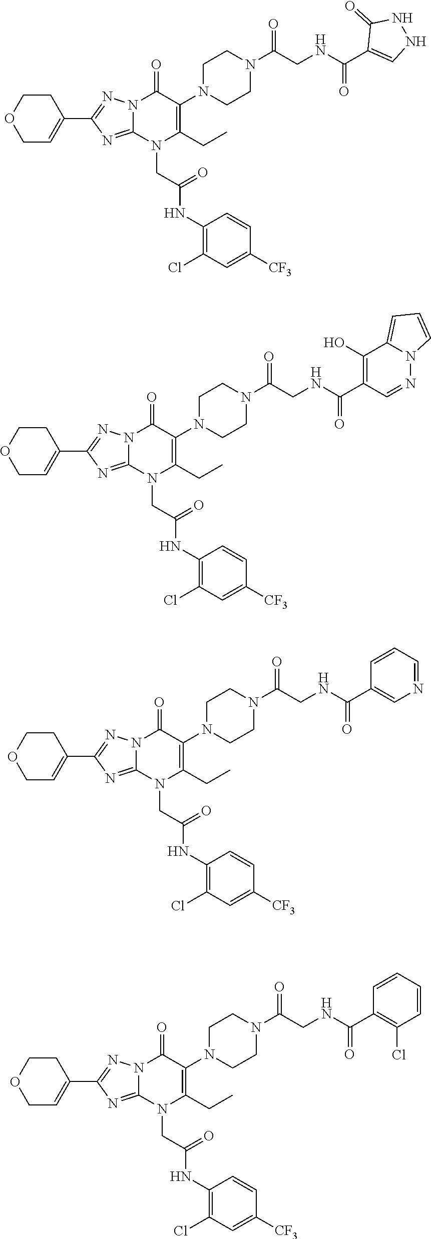 [SMILES: CCc1c(N2CCN(C(=O)CNC(=O)c3c[nH][nH]c3=O)CC2)c(=O)n2nc(C3=CCOCC3)nc2n1CC(=O)Nc1ccc(C(F)(F)F)cc1Cl ; CCc1c(N2CCN(C(=O)CNC(=O)c3cnn4cccc4c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cccnc3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3Cl)CC2)c(=O)n2nc(C3=CCOCC3)nc2n1CC(=O)Nc1ccc(C(F)(F)F)cc1Cl] 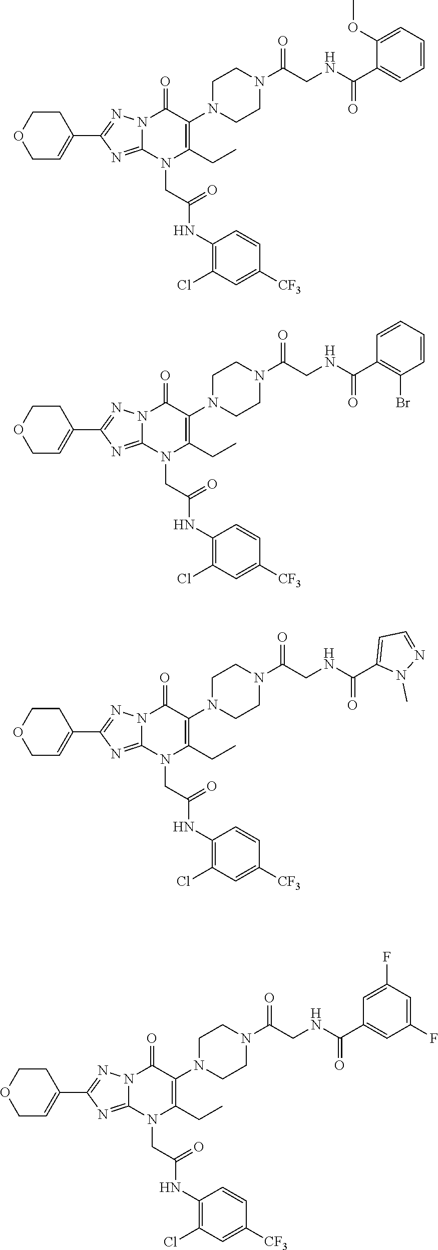 [SMILES: CCc1c(N2CCN(C(=O)CNC(=O)c3ccccc3OC)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3Br)CC2)c(=O)n2nc(C3=CCOCC3)nc2n1CC(=O)Nc1ccc(C(F)(F)F)cc1Cl ; CCc1c(N2CCN(C(=O)CNC(=O)c3ccnn3C)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(F)cc(F)c3)CC2)c(=O)n2nc(C3=CCOCC3)nc2n1CC(=O)Nc1ccc(C(F)(F)F)cc1Cl] 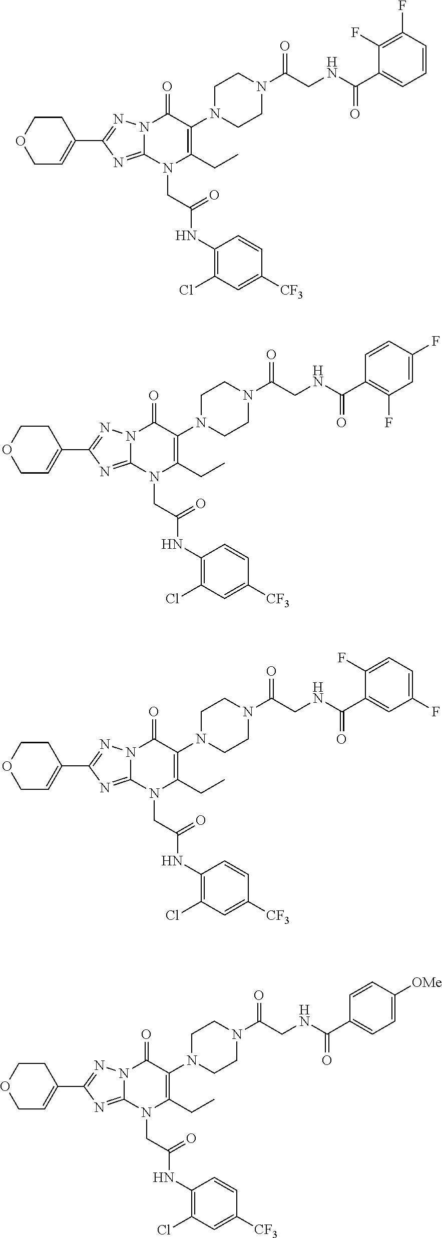 [SMILES: CCc1c(N2CCN(C(=O)CNC(=O)c3cccc(F)c3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)cc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(F)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(OC)cc3)CC2)c(=O)n2nc(C3=CCOCC3)nc2n1CC(=O)Nc1ccc(C(F)(F)F)cc1Cl] 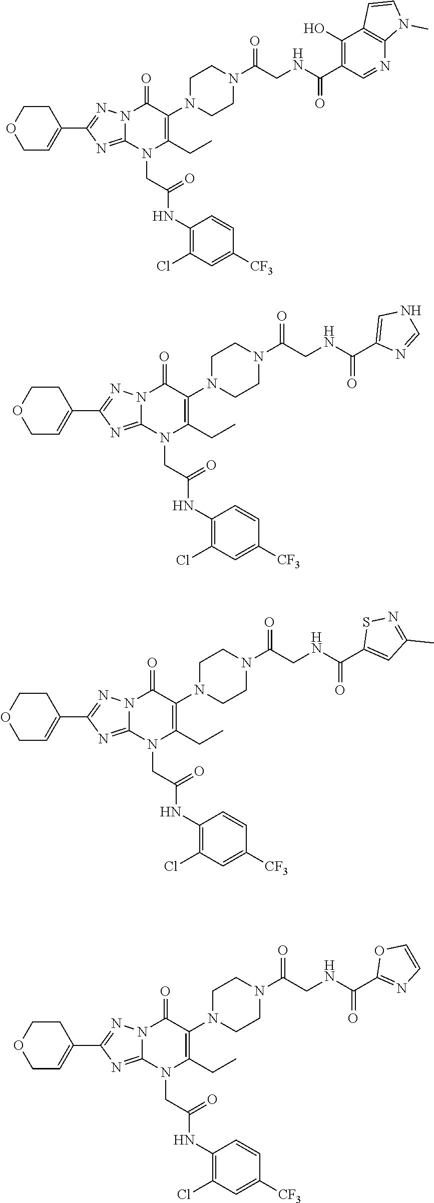 [SMILES: CCc1c(N2CCN(C(=O)CNC(=O)c3cnc4c(ccn4C)c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3c[nH]cn3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C)ns3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncco3)CC2)c(=O)n2nc(C3=CCOCC3)nc2n1CC(=O)Nc1ccc(C(F)(F)F)cc1Cl] 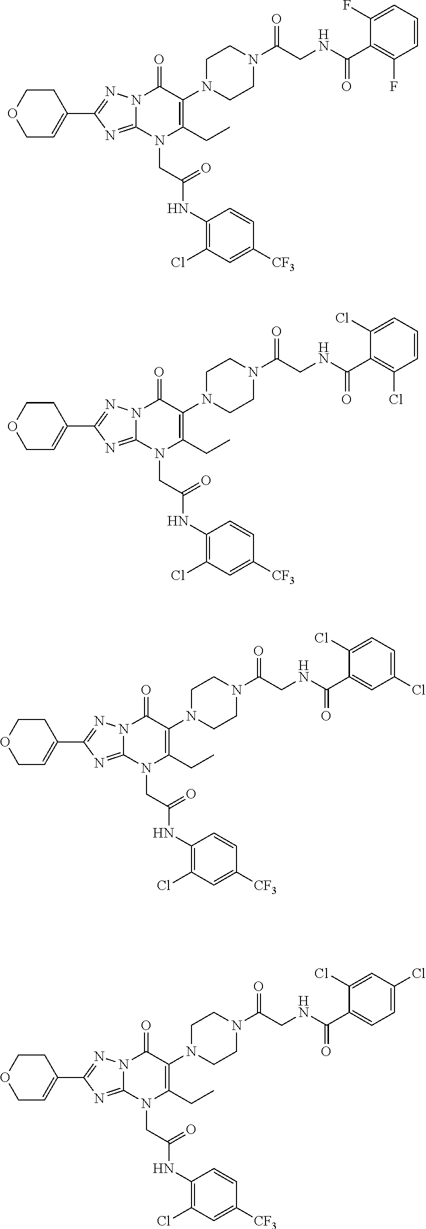 [SMILES: CCc1c(N2CCN(C(=O)CNC(=O)c3c(F)cccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3c(Cl)cccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(Cl)ccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(Cl)cc3Cl)CC2)c(=O)n2nc(C3=CCOCC3)nc2n1CC(=O)Nc1ccc(C(F)(F)F)cc1Cl] 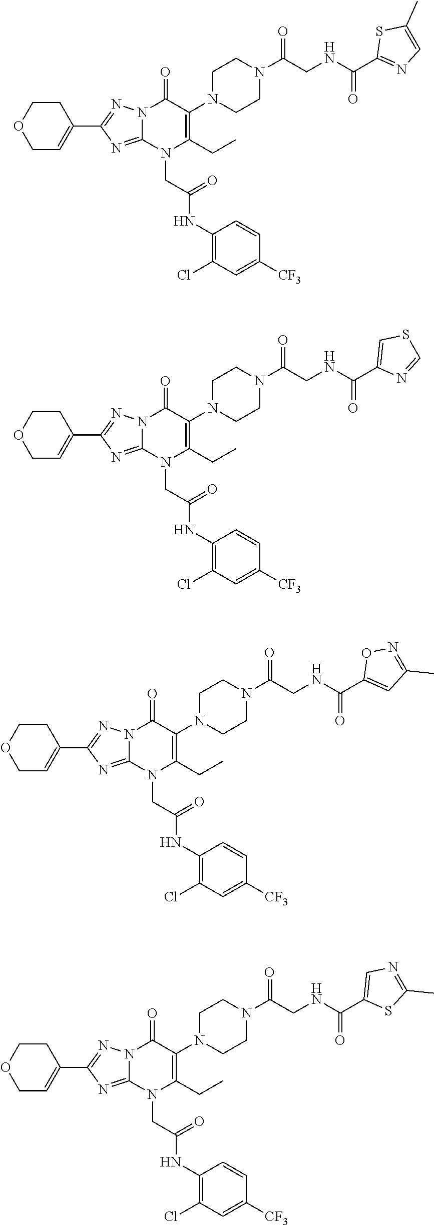 [SMILES: CCc1c(N2CCN(C(=O)CNC(=O)c3ncc(C)s3)CC2)c(=O)n2nc(C3=CCOCC3)nc2n1CC(=O)Nc1ccc(C(F)(F)F)cc1Cl ; CCc1c(N2CCN(C(=O)CNC(=O)c3cscn3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C)no3)CC2)c(=O)n2nc(C3=CCOCC3)nc2n1CC(=O)Nc1ccc(C(F)(F)F)cc1Cl ; CCc1c(N2CCN(C(=O)CNC(=O)c3cnc(C)s3)CC2)c(=O)n2nc(C3=CCOCC3)nc2n1CC(=O)Nc1ccc(C(F)(F)F)cc1Cl] 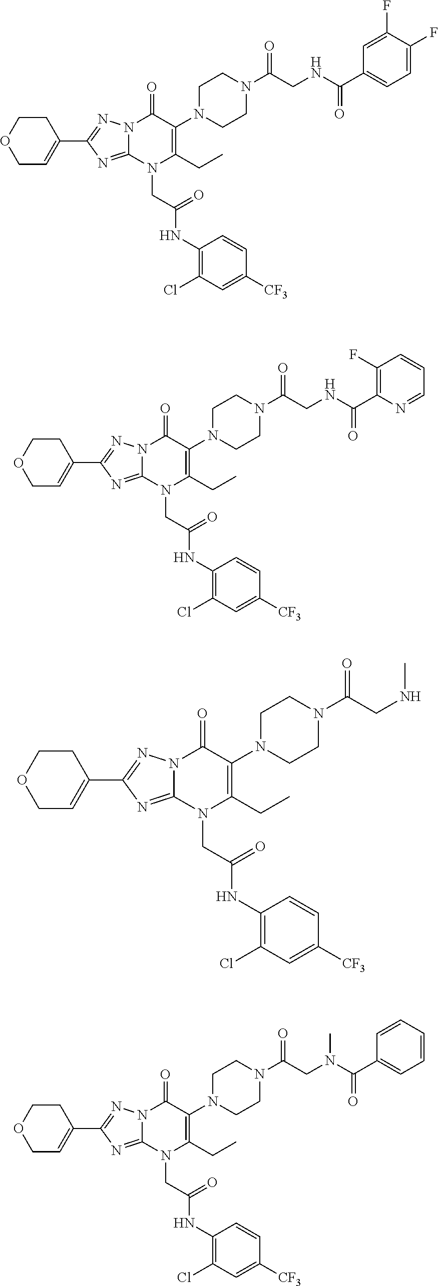 [SMILES: CCc1c(N2CCN(C(=O)CNC(=O)c3ccc(F)c(F)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncccc3F)CC2)c(=O)n2nc(C3=CCOCC3)nc2n1CC(=O)Nc1ccc(C(F)(F)F)cc1Cl ; CCc1c(N2CCN(C(=O)CNC)CC2)c(=O)n2nc(C3=CCOCC3)nc2n1CC(=O)Nc1ccc(C(F)(F)F)cc1Cl ; CCc1c(N2CCN(C(=O)CN(C)C(=O)c3ccccc3)CC2)c(=O)n2nc(C3=CCOCC3)nc2n1CC(=O)Nc1ccc(C(F)(F)F)cc1Cl] 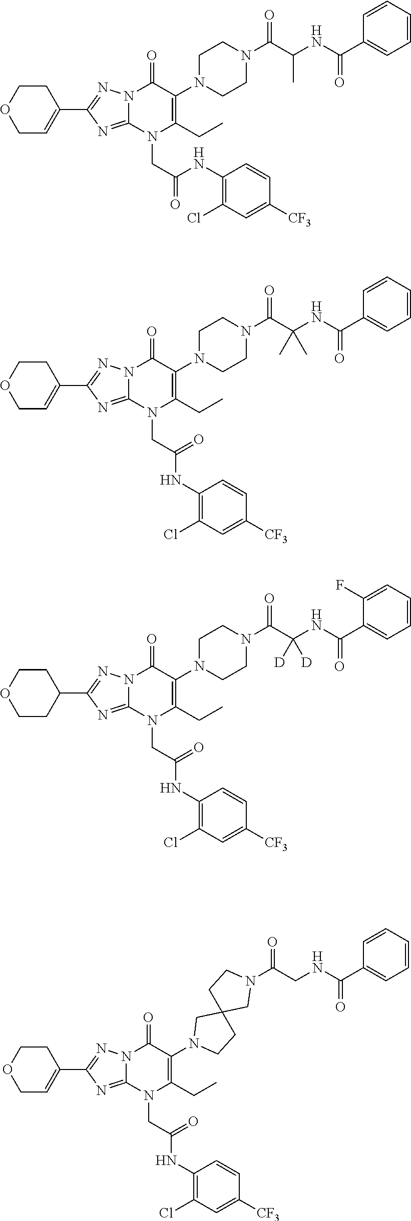 [SMILES: CCc1c(N2CCN(C(=O)C(C)NC(=O)c3ccccc3)CC2)c(=O)n2nc(C3=CCOCC3)nc2n1CC(=O)Nc1ccc(C(F)(F)F)cc1Cl ; CCc1c(N2CCN(C(=O)C(C)(C)NC(=O)c3ccccc3)CC2)c(=O)n2nc(C3=CCOCC3)nc2n1CC(=O)Nc1ccc(C(F)(F)F)cc1Cl ; [2H]C([2H])(NC(=O)c1ccccc1F)C(=O)N1CCN(c2c(CC)n(CC(=O)Nc3ccc(C(F)(F)F)cc3Cl)c3nc(C4CCOCC4)nn3c2=O)CC1 ; CCc1c(N2CCC3(CCN(C(=O)CNC(=O)c4ccccc4)C3)C2)c(=O)n2nc(C3=CCOCC3)nc2n1CC(=O)Nc1ccc(C(F)(F)F)cc1Cl] 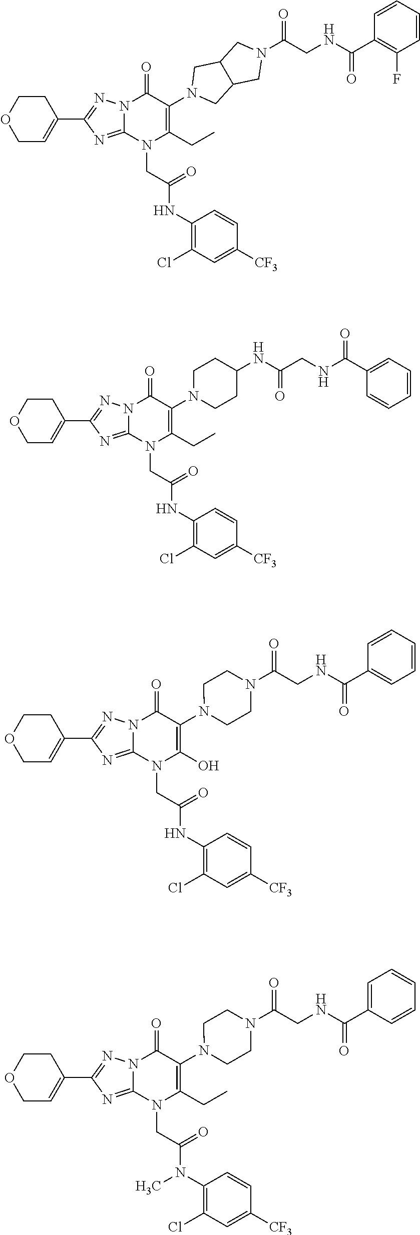 [SMILES: CCc1c(N2CC3CN(C(=O)CNC(=O)c4ccccc4F)CC3C2)c(=O)n2nc(C3=CCOCC3)nc2n1CC(=O)Nc1ccc(C(F)(F)F)cc1Cl ; CCc1c(N2CCC(NC(=O)CNC(=O)c3ccccc3)CC2)c(=O)n2nc(C3=CCOCC3)nc2n1CC(=O)Nc1ccc(C(F)(F)F)cc1Cl ; O=C(Cn1c(O)c(N2CCN(C(=O)CNC(=O)c3ccccc3)CC2)c(=O)n2nc(C3=CCOCC3)nc12)Nc1ccc(C(F)(F)F)cc1Cl ; CCc1c(N2CCN(C(=O)CNC(=O)c3ccccc3)CC2)c(=O)n2nc(C3=CCOCC3)nc2n1CC(=O)N(C)c1ccc(C(F)(F)F)cc1Cl] 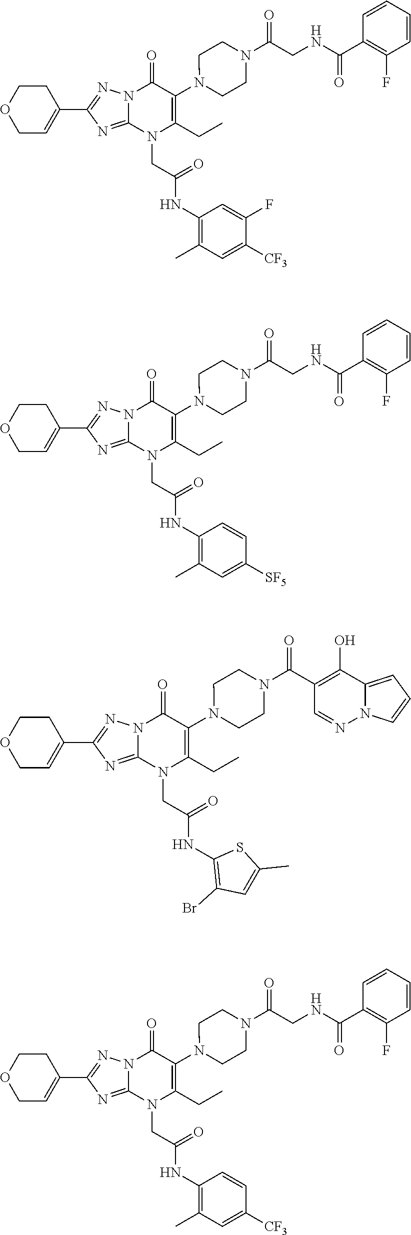 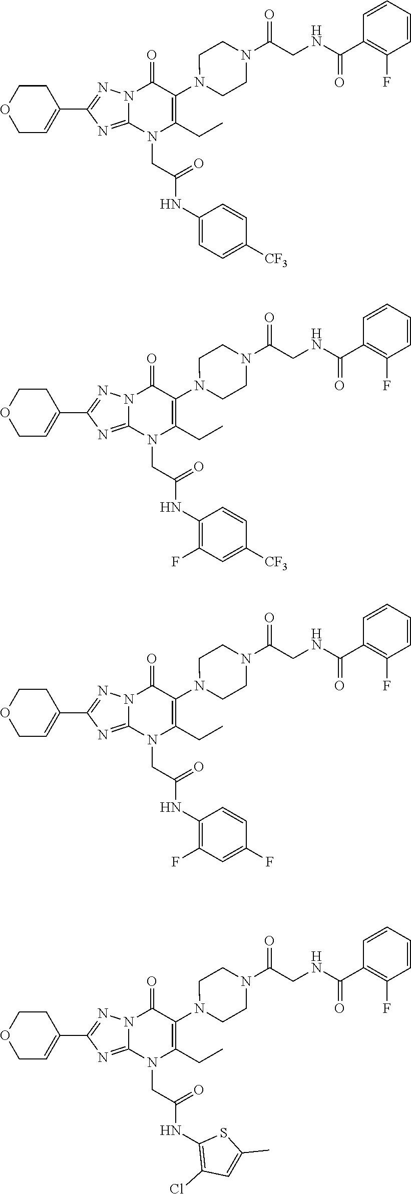 [SMILES: CCc1c(N2CCN(C(=O)CNC(=O)c3ccccc3F)CC2)c(=O)n2nc(C3=CCOCC3)nc2n1CC(=O)Nc1ccc(C(F)(F)F)cc1 ; CCc1c(N2CCN(C(=O)CNC(=O)c3ccccc3F)CC2)c(=O)n2nc(C3=CCOCC3)nc2n1CC(=O)Nc1ccc(C(F)(F)F)cc1F ; CCc1c(N2CCN(C(=O)CNC(=O)c3ccccc3F)CC2)c(=O)n2nc(C3=CCOCC3)nc2n1CC(=O)Nc1ccc(F)cc1F ; CCc1c(N2CCN(C(=O)CNC(=O)c3ccccc3F)CC2)c(=O)n2nc(C3=CCOCC3)nc2n1CC(=O)Nc1sc(C)cc1Cl] 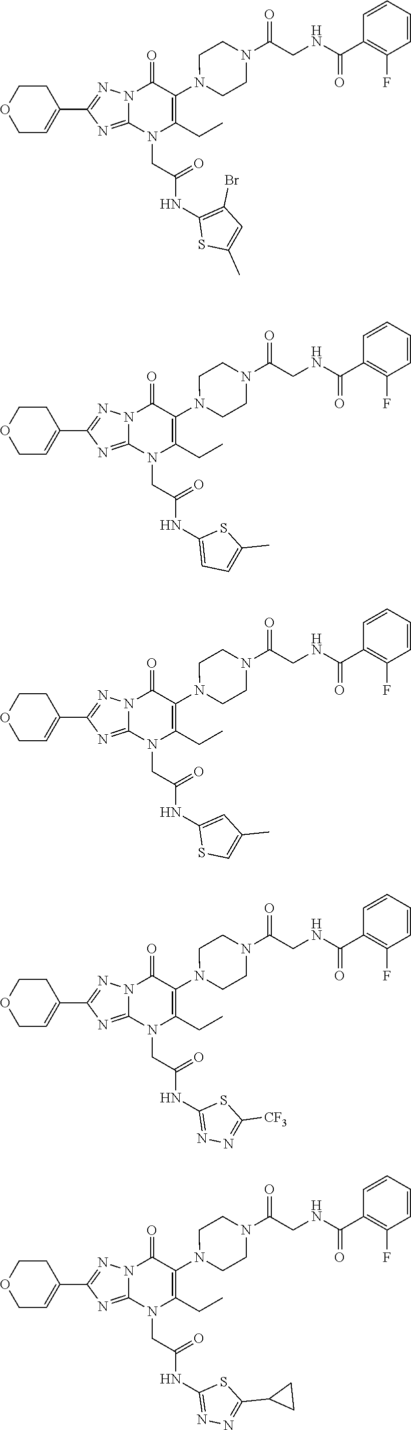 [SMILES: CCc1c(N2CCN(C(=O)CNC(=O)c3ccccc3F)CC2)c(=O)n2nc(C3=CCOCC3)nc2n1CC(=O)Nc1sc(C)cc1Br ; CCc1c(N2CCN(C(=O)CNC(=O)c3ccccc3F)CC2)c(=O)n2nc(C3=CCOCC3)nc2n1CC(=O)Nc1ccc(C)s1 ; CCc1c(N2CCN(C(=O)CNC(=O)c3ccccc3F)CC2)c(=O)n2nc(C3=CCOCC3)nc2n1CC(=O)Nc1cc(C)cs1 ; CCc1c(N2CCN(C(=O)CNC(=O)c3ccccc3F)CC2)c(=O)n2nc(C3=CCOCC3)nc2n1CC(=O)Nc1nnc(C(F)(F)F)s1 ; CCc1c(N2CCN(C(=O)CNC(=O)c3ccccc3F)CC2)c(=O)n2nc(C3=CCOCC3)nc2n1CC(=O)Nc1nnc(C2CC2)s1] 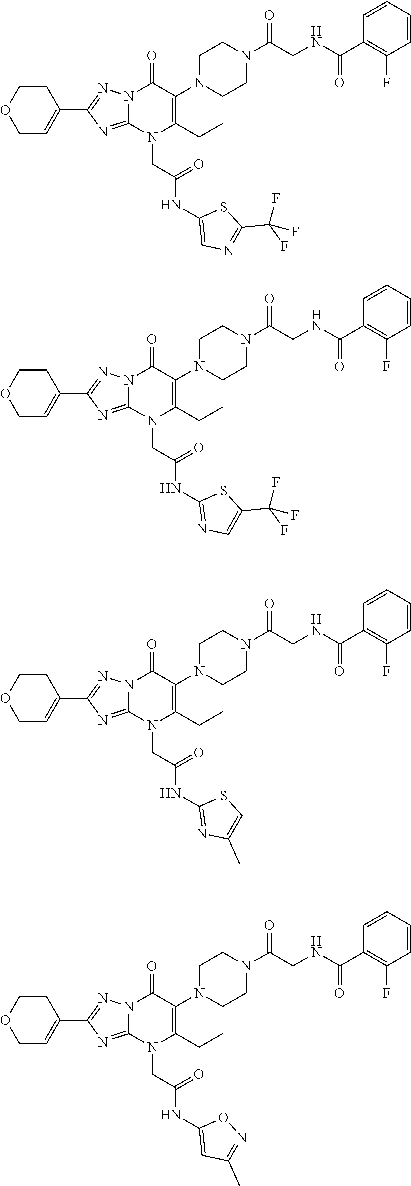 [SMILES: CCc1c(N2CCN(C(=O)CNC(=O)c3ccccc3F)CC2)c(=O)n2nc(C3=CCOCC3)nc2n1CC(=O)Nc1cnc(C(F)(F)F)s1 ; CCc1c(N2CCN(C(=O)CNC(=O)c3ccccc3F)CC2)c(=O)n2nc(C3=CCOCC3)nc2n1CC(=O)Nc1ncc(C(F)(F)F)s1 ; CCc1c(N2CCN(C(=O)CNC(=O)c3ccccc3F)CC2)c(=O)n2nc(C3=CCOCC3)nc2n1CC(=O)Nc1nc(C)cs1 ; CCc1c(N2CCN(C(=O)CNC(=O)c3ccccc3F)CC2)c(=O)n2nc(C3=CCOCC3)nc2n1CC(=O)Nc1cc(C)no1] 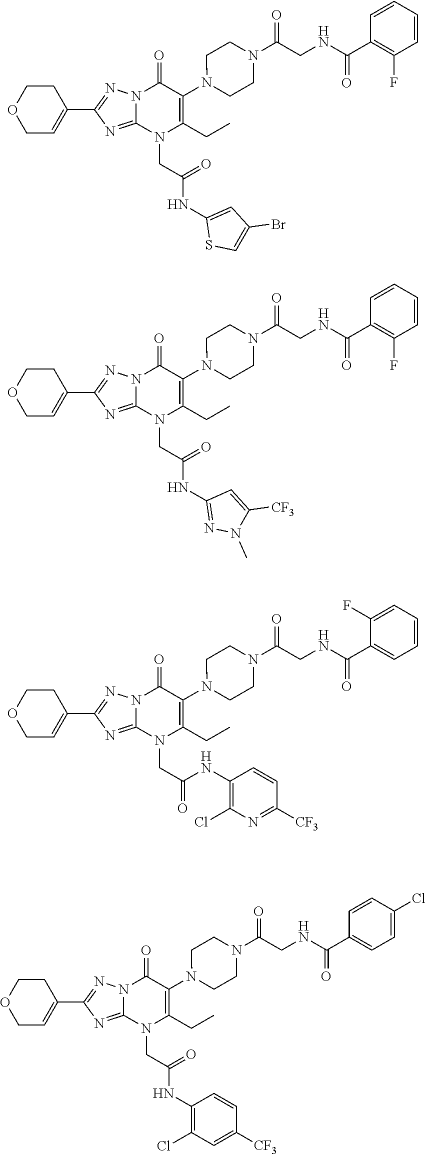 [SMILES: CCc1c(N2CCN(C(=O)CNC(=O)c3ccccc3F)CC2)c(=O)n2nc(C3=CCOCC3)nc2n1CC(=O)Nc1cc(Br)cs1 ; CCc1c(N2CCN(C(=O)CNC(=O)c3ccccc3F)CC2)c(=O)n2nc(C3=CCOCC3)nc2n1CC(=O)Nc1cc(C(F)(F)F)n(C)n1 ; CCc1c(N2CCN(C(=O)CNC(=O)c3ccccc3F)CC2)c(=O)n2nc(C3=CCOCC3)nc2n1CC(=O)Nc1ccc(C(F)(F)F)nc1Cl ; CCc1c(N2CCN(C(=O)CNC(=O)c3ccc(Cl)cc3)CC2)c(=O)n2nc(C3=CCOCC3)nc2n1CC(=O)Nc1ccc(C(F)(F)F)cc1Cl] 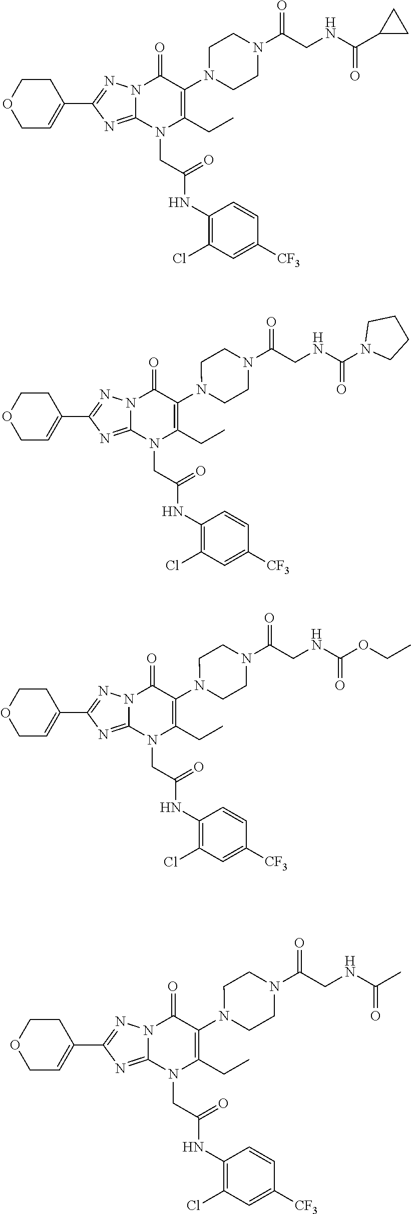 [SMILES: CCc1c(N2CCN(C(=O)CNC(=O)C3CC3)CC2)c(=O)n2nc(C3=CCOCC3)nc2n1CC(=O)Nc1ccc(C(F)(F)F)cc1Cl ; CCc1c(N2CCN(C(=O)CNC(=O)N3CCCC3)CC2)c(=O)n2nc(C3=CCOCC3)nc2n1CC(=O)Nc1ccc(C(F)(F)F)cc1Cl ; CCOC(=O)NCC(=O)N1CCN(c2c(CC)n(CC(=O)Nc3ccc(C(F)(F)F)cc3Cl)c3nc(C4=CCOCC4)nn3c2=O)CC1 ; CCc1c(N2CCN(C(=O)CNC(C)=O)CC2)c(=O)n2nc(C3=CCOCC3)nc2n1CC(=O)Nc1ccc(C(F)(F)F)cc1Cl] 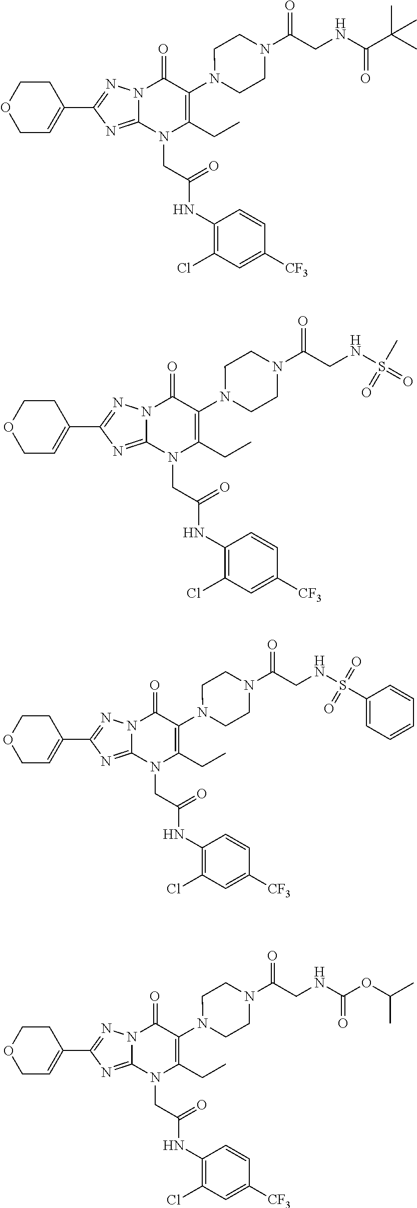 [SMILES: CCc1c(N2CCN(C(=O)CNC(=O)C(C)(C)C)CC2)c(=O)n2nc(C3=CCOCC3)nc2n1CC(=O)Nc1ccc(C(F)(F)F)cc1Cl ; CCc1c(N2CCN(C(=O)CNS(C)(=O)=O)CC2)c(=O)n2nc(C3=CCOCC3)nc2n1CC(=O)Nc1ccc(C(F)(F)F)cc1Cl ; CCc1c(N2CCN(C(=O)CNS(=O)(=O)c3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)OC(C)C)CC2)c(=O)n2nc(C3=CCOCC3)nc2n1CC(=O)Nc1ccc(C(F)(F)F)cc1Cl] 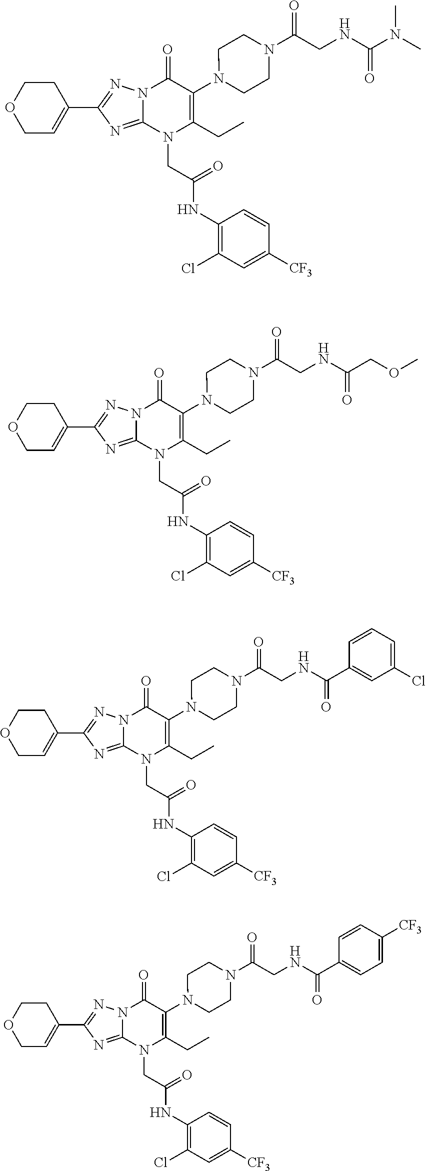 [SMILES: CCc1c(N2CCN(C(=O)CNC(=O)N(C)C)CC2)c(=O)n2nc(C3=CCOCC3)nc2n1CC(=O)Nc1ccc(C(F)(F)F)cc1Cl ; CCc1c(N2CCN(C(=O)CNC(=O)COC)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(Cl)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(C(F)(F)F)cc3)CC2)c(=O)n2nc(C3=CCOCC3)nc2n1CC(=O)Nc1ccc(C(F)(F)F)cc1Cl] 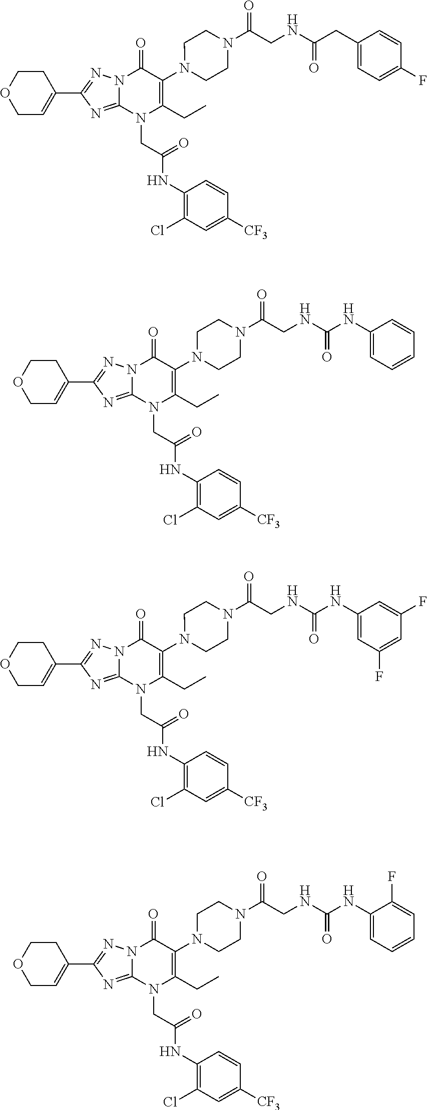 [SMILES: CCc1c(N2CCN(C(=O)CNC(=O)Cc3ccc(F)cc3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3cc(F)cc(F)c3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3ccccc3F)CC2)c(=O)n2nc(C3=CCOCC3)nc2n1CC(=O)Nc1ccc(C(F)(F)F)cc1Cl] 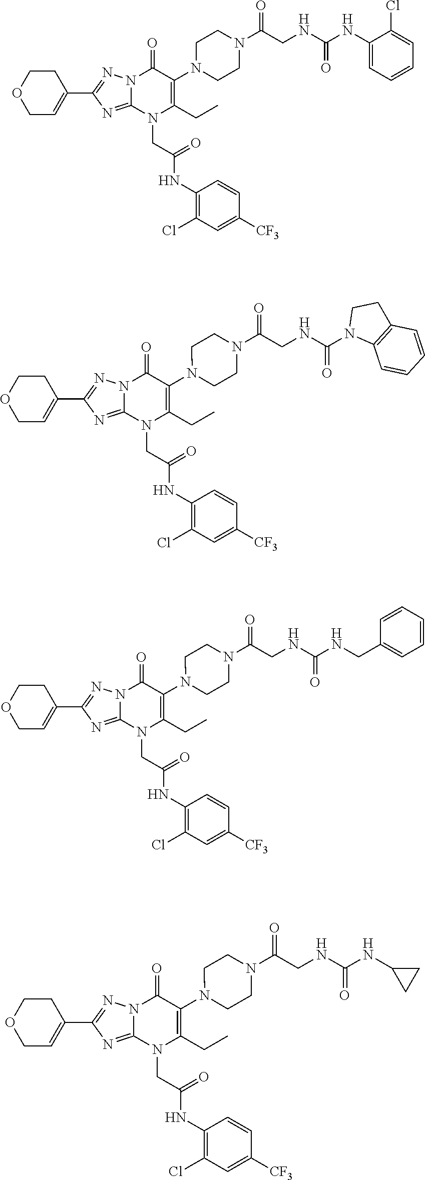 [SMILES: CCc1c(N2CCN(C(=O)CNC(=O)Nc3ccccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)N3CCc4ccccc43)CC2)c(=O)n2nc(C3=CCOCC3)nc2n1CC(=O)Nc1ccc(C(F)(F)F)cc1Cl ; CCc1c(N2CCN(C(=O)CNC(=O)NCc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)NC3CC3)CC2)c(=O)n2nc(C3=CCOCC3)nc2n1CC(=O)Nc1ccc(C(F)(F)F)cc1Cl] 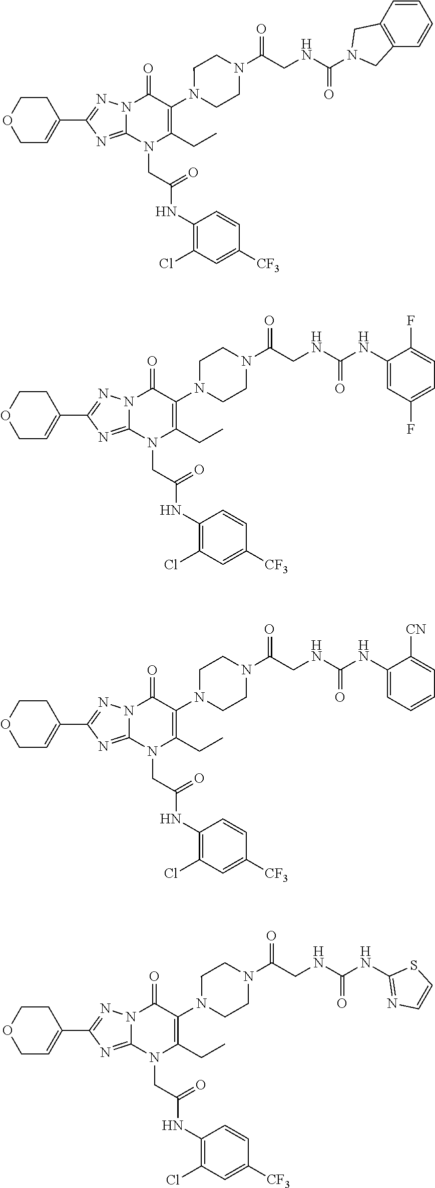 [SMILES: CCc1c(N2CCN(C(=O)CNC(=O)N3Cc4ccccc4C3)CC2)c(=O)n2nc(C3=CCOCC3)nc2n1CC(=O)Nc1ccc(C(F)(F)F)cc1Cl ; CCc1c(N2CCN(C(=O)CNC(=O)Nc3cc(F)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)Nc3ccccc3C#N)CC2)c(=O)n2nc(C3=CCOCC3)nc2n1CC(=O)Nc1ccc(C(F)(F)F)cc1Cl ; CCc1c(N2CCN(C(=O)CNC(=O)Nc3nccs3)CC2)c(=O)n2nc(C3=CCOCC3)nc2n1CC(=O)Nc1ccc(C(F)(F)F)cc1Cl] 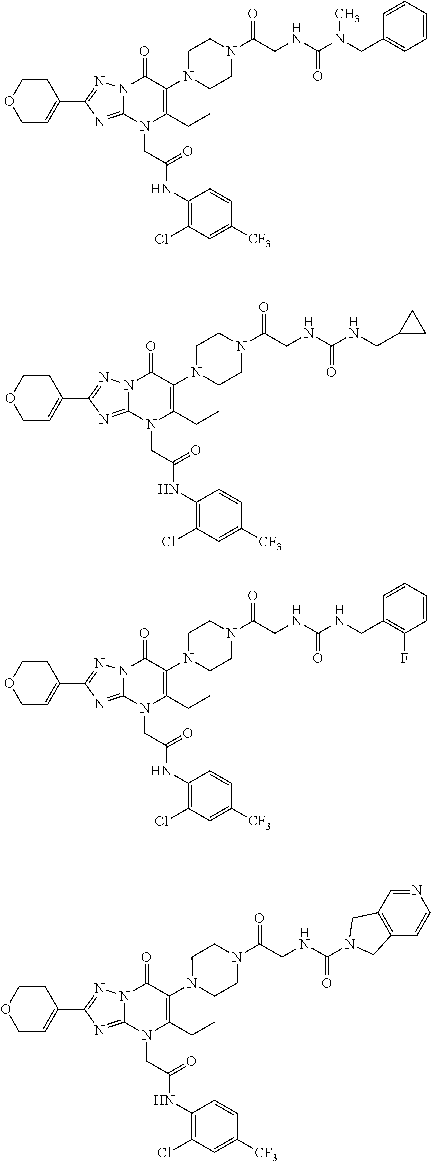 [SMILES: CCc1c(N2CCN(C(=O)CNC(=O)N(C)Cc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)NCC3CC3)CC2)c(=O)n2nc(C3=CCOCC3)nc2n1CC(=O)Nc1ccc(C(F)(F)F)cc1Cl ; CCc1c(N2CCN(C(=O)CNC(=O)NCc3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)N3Cc4ccncc4C3)CC2)c(=O)n2nc(C3=CCOCC3)nc2n1CC(=O)Nc1ccc(C(F)(F)F)cc1Cl] 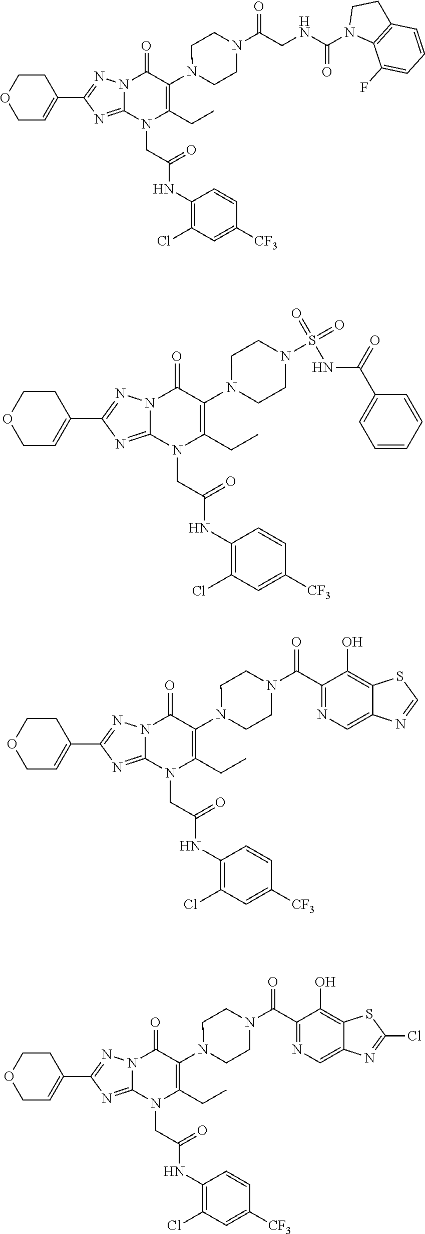 [SMILES: CCc1c(N2CCN(C(=O)CNC(=O)N3CCc4cccc(F)c43)CC2)c(=O)n2nc(C3=CCOCC3)nc2n1CC(=O)Nc1ccc(C(F)(F)F)cc1Cl ; CCc1c(N2CCN(S(=O)(=O)NC(=O)c3ccccc3)CC2)c(=O)n2nc(C3=CCOCC3)nc2n1CC(=O)Nc1ccc(C(F)(F)F)cc1Cl ; CCc1c(N2CCN(C(=O)c3ncc4ncsc4c3O)CC2)c(=O)n2nc(C3=CCOCC3)nc2n1CC(=O)Nc1ccc(C(F)(F)F)cc1Cl ; CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1Cl] 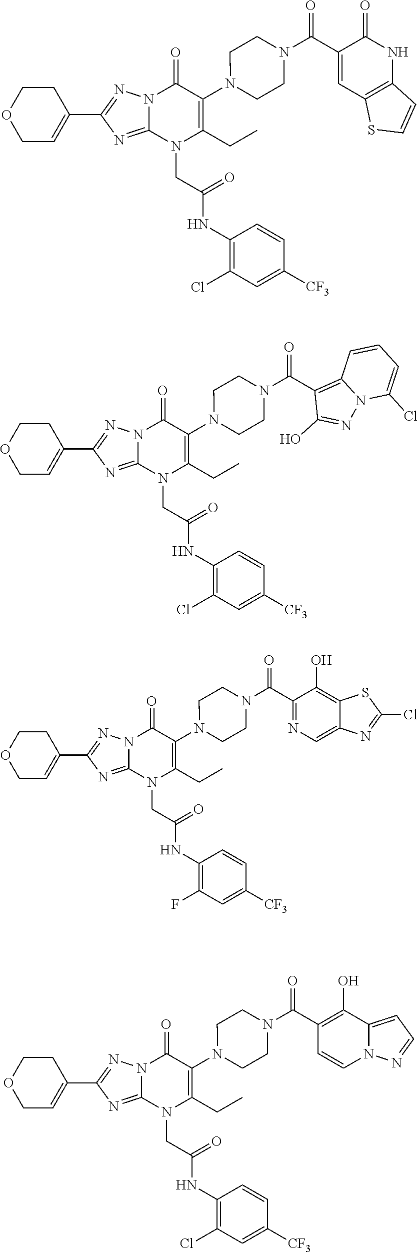 [SMILES: CCc1c(N2CCN(C(=O)c3cc4sccc4[nH]c3=O)CC2)c(=O)n2nc(C3=CCOCC3)nc2n1CC(=O)Nc1ccc(C(F)(F)F)cc1Cl ; CCc1c(N2CCN(C(=O)c3c(O)nn4c(Cl)cccc34)CC2)c(=O)n2nc(C3=CCOCC3)nc2n1CC(=O)Nc1ccc(C(F)(F)F)cc1Cl ; CCc1c(N2CCN(C(=O)c3ncc4nc(Cl)sc4c3O)CC2)c(=O)n2nc(C3=CCOCC3)nc2n1CC(=O)Nc1ccc(C(F)(F)F)cc1F ; CCc1c(N2CCN(C(=O)c3ccn4nccc4c3O)CC2)c(=O)n2nc(C3=CCOCC3)nc2n1CC(=O)Nc1ccc(C(F)(F)F)cc1Cl] 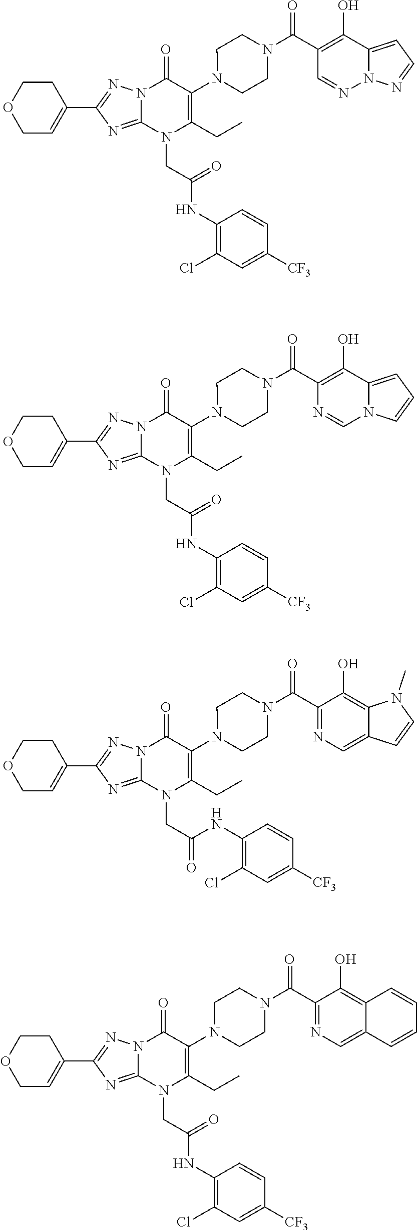 [SMILES: CCc1c(N2CCN(C(=O)c3cnn4nccc4c3O)CC2)c(=O)n2nc(C3=CCOCC3)nc2n1CC(=O)Nc1ccc(C(F)(F)F)cc1Cl ; CCc1c(N2CCN(C(=O)c3ncn4cccc4c3O)CC2)c(=O)n2nc(C3=CCOCC3)nc2n1CC(=O)Nc1ccc(C(F)(F)F)cc1Cl ; CCc1c(N2CCN(C(=O)c3ncc4ccn(C)c4c3O)CC2)c(=O)n2nc(C3=CCOCC3)nc2n1CC(=O)Nc1ccc(C(F)(F)F)cc1Cl ; CCc1c(N2CCN(C(=O)c3ncc4ccccc4c3O)CC2)c(=O)n2nc(C3=CCOCC3)nc2n1CC(=O)Nc1ccc(C(F)(F)F)cc1Cl] 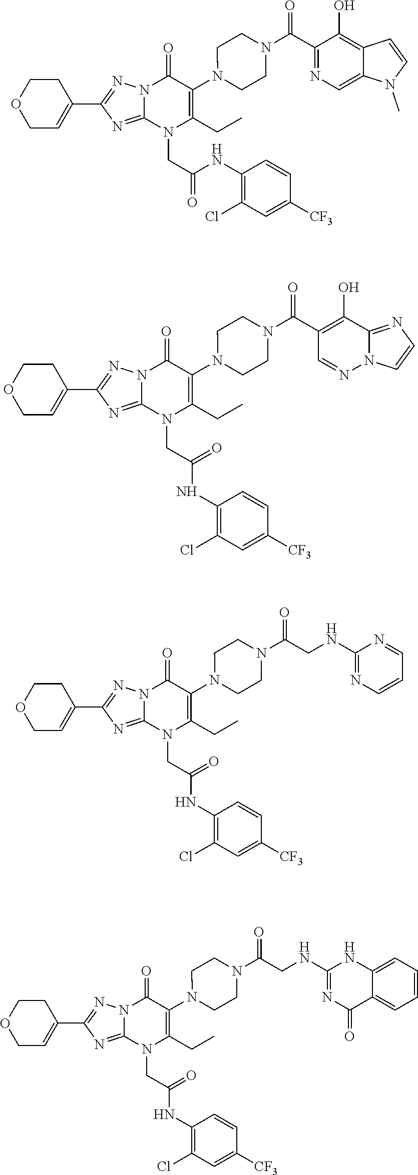 [SMILES: CCc1c(N2CCN(C(=O)c3ncc4c(ccn4C)c3O)CC2)c(=O)n2nc(C3=CCOCC3)nc2n1CC(=O)Nc1ccc(C(F)(F)F)cc1Cl ; CCc1c(N2CCN(C(=O)c3cnn4ccnc4c3O)CC2)c(=O)n2nc(C3=CCOCC3)nc2n1CC(=O)Nc1ccc(C(F)(F)F)cc1Cl ; CCc1c(N2CCN(C(=O)CNc3ncccn3)CC2)c(=O)n2nc(C3=CCOCC3)nc2n1CC(=O)Nc1ccc(C(F)(F)F)cc1Cl ; CCc1c(N2CCN(C(=O)CNc3nc(=O)c4ccccc4[nH]3)CC2)c(=O)n2nc(C3=CCOCC3)nc2n1CC(=O)Nc1ccc(C(F)(F)F)cc1Cl] 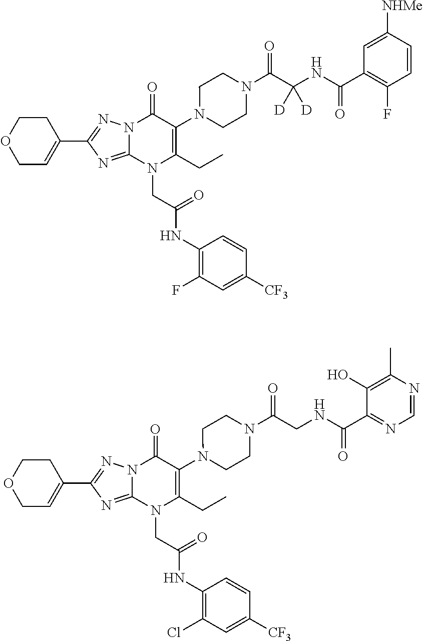 [SMILES: [2H]C([2H])(NC(=O)c1cc(NC)ccc1F)C(=O)N1CCN(c2c(CC)n(CC(=O)Nc3ccc(C(F)(F)F)cc3F)c3nc(C4=CCOCC4)nn3c2=O)CC1 ; CCc1c(N2CCN(C(=O)CNC(=O)c3ncnc(C)c3O)CC2)c(=O)n2nc(C3=CCOCC3)nc2n1CC(=O)Nc1ccc(C(F)(F)F)cc1Cl] 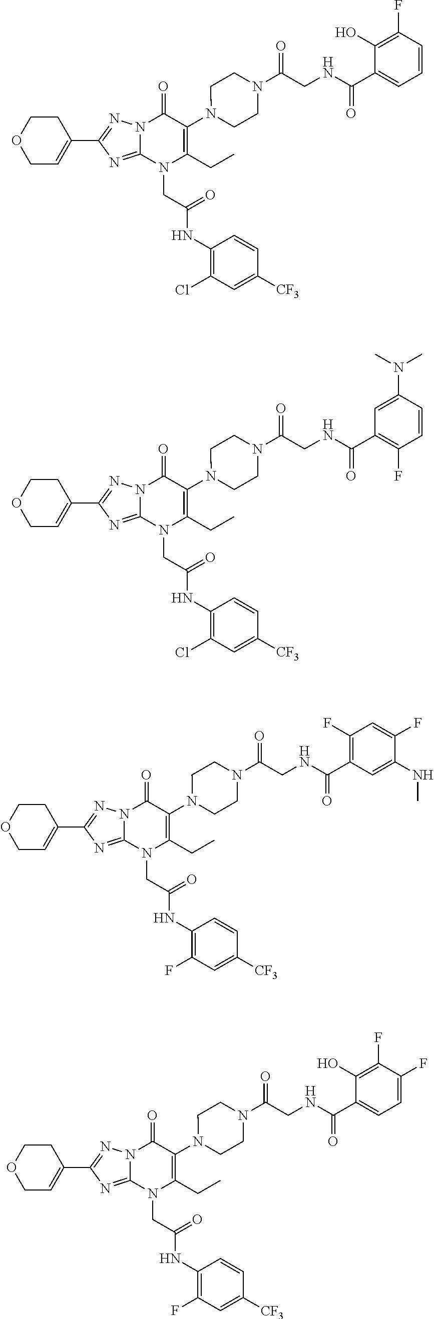 [SMILES: CCc1c(N2CCN(C(=O)CNC(=O)c3cccc(F)c3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(N(C)C)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(NC)c(F)cc3F)CC2)c(=O)n2nc(C3=CCOCC3)nc2n1CC(=O)Nc1ccc(C(F)(F)F)cc1F ; CCc1c(N2CCN(C(=O)CNC(=O)c3ccc(F)c(F)c3O)CC2)c(=O)n2nc(C3=CCOCC3)nc2n1CC(=O)Nc1ccc(C(F)(F)F)cc1F] 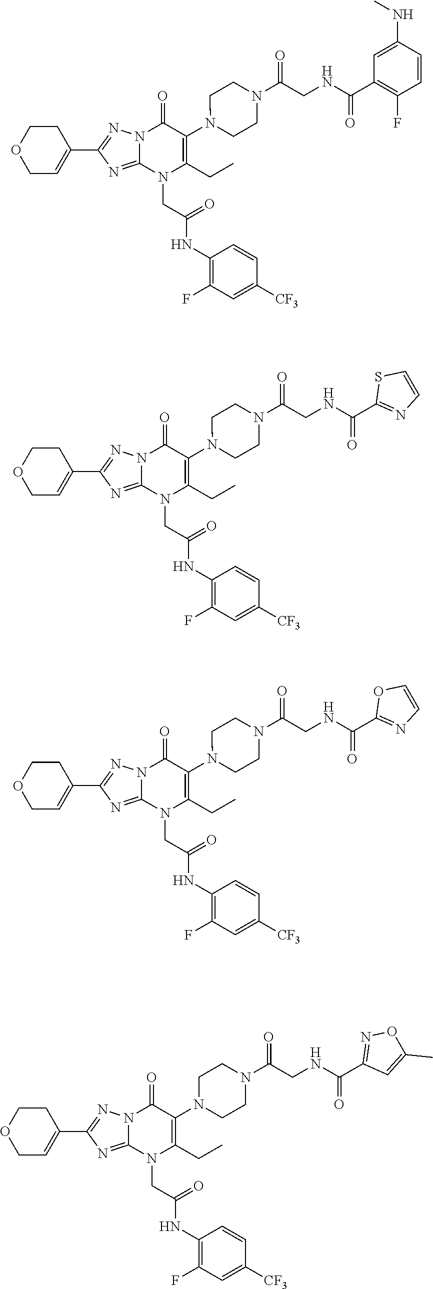 [SMILES: CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1F ; CCc1c(N2CCN(C(=O)CNC(=O)c3nccs3)CC2)c(=O)n2nc(C3=CCOCC3)nc2n1CC(=O)Nc1ccc(C(F)(F)F)cc1F ; CCc1c(N2CCN(C(=O)CNC(=O)c3ncco3)CC2)c(=O)n2nc(C3=CCOCC3)nc2n1CC(=O)Nc1ccc(C(F)(F)F)cc1F ; CCc1c(N2CCN(C(=O)CNC(=O)c3cc(C)on3)CC2)c(=O)n2nc(C3=CCOCC3)nc2n1CC(=O)Nc1ccc(C(F)(F)F)cc1F] 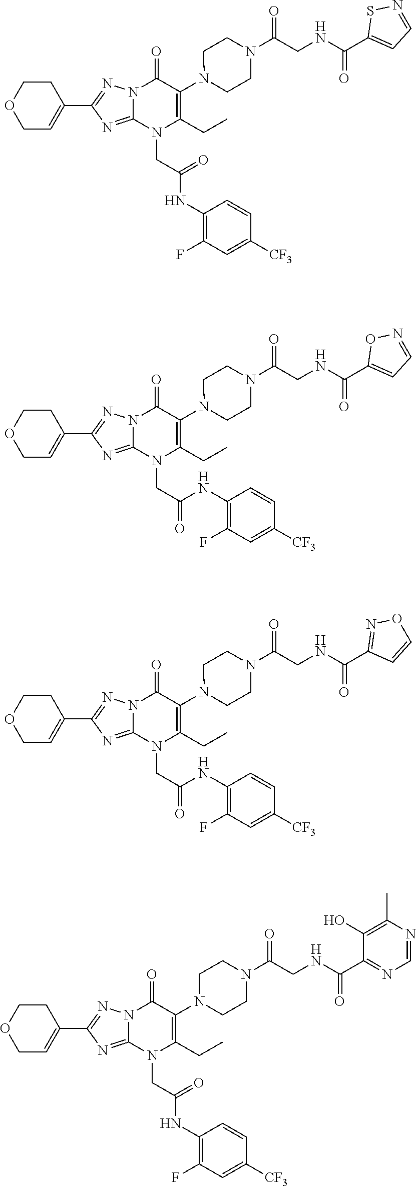 [SMILES: CCc1c(N2CCN(C(=O)CNC(=O)c3ccns3)CC2)c(=O)n2nc(C3=CCOCC3)nc2n1CC(=O)Nc1ccc(C(F)(F)F)cc1F ; CCc1c(N2CCN(C(=O)CNC(=O)c3ccno3)CC2)c(=O)n2nc(C3=CCOCC3)nc2n1CC(=O)Nc1ccc(C(F)(F)F)cc1F ; CCc1c(N2CCN(C(=O)CNC(=O)c3ccon3)CC2)c(=O)n2nc(C3=CCOCC3)nc2n1CC(=O)Nc1ccc(C(F)(F)F)cc1F ; CCc1c(N2CCN(C(=O)CNC(=O)c3ncnc(C)c3O)CC2)c(=O)n2nc(C3=CCOCC3)nc2n1CC(=O)Nc1ccc(C(F)(F)F)cc1F] 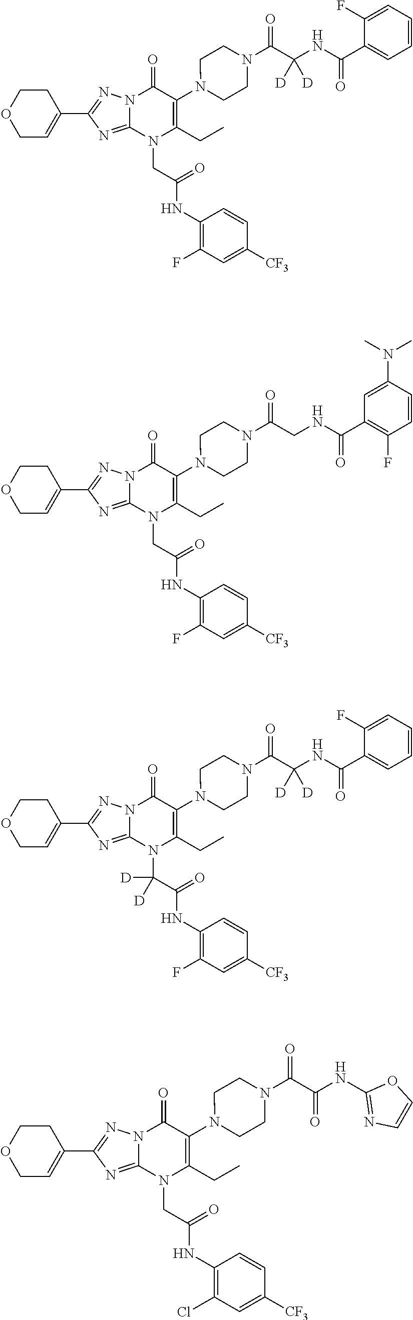 [SMILES: [2H]C([2H])(NC(=O)c1ccccc1F)C(=O)N1CCN(c2c(CC)n(CC(=O)Nc3ccc(C(F)(F)F)cc3F)c3nc(C4=CCOCC4)nn3c2=O)CC1 ; CCc1c(N2CCN(C(=O)CNC(=O)c3cc(N(C)C)ccc3F)CC2)c(=O)n2nc(C3=CCOCC3)nc2n1CC(=O)Nc1ccc(C(F)(F)F)cc1F ; [2H]C([2H])(NC(=O)c1ccccc1F)C(=O)N1CCN(c2c(CC)n(C([2H])([2H])C(=O)Nc3ccc(C(F)(F)F)cc3F)c3nc(C4=CCOCC4)nn3c2=O)CC1 ; CCc1c(N2CCN(C(=O)C(=O)Nc3ncco3)CC2)c(=O)n2nc(C3=CCOCC3)nc2n1CC(=O)Nc1ccc(C(F)(F)F)cc1Cl] 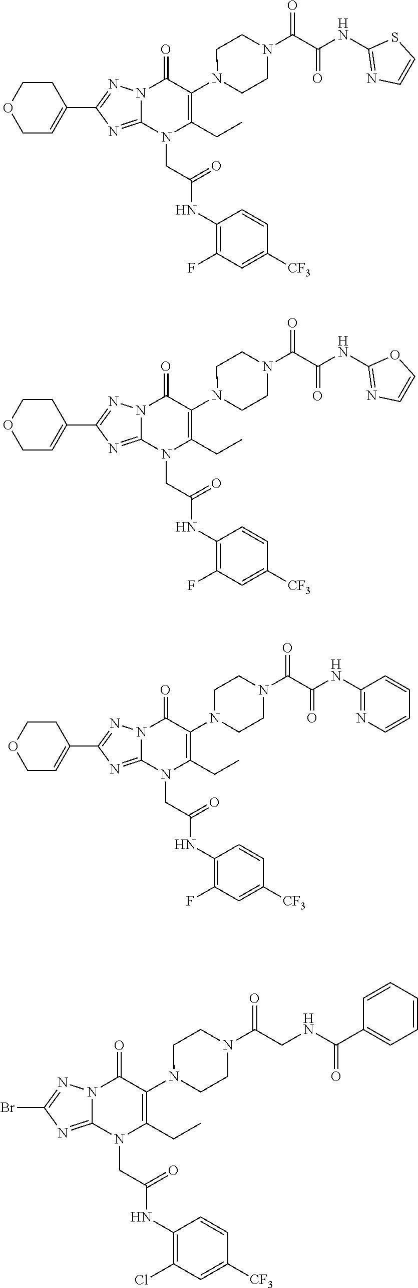 [SMILES: CCc1c(N2CCN(C(=O)C(=O)Nc3nccs3)CC2)c(=O)n2nc(C3=CCOCC3)nc2n1CC(=O)Nc1ccc(C(F)(F)F)cc1F ; CCc1c(N2CCN(C(=O)C(=O)Nc3ncco3)CC2)c(=O)n2nc(C3=CCOCC3)nc2n1CC(=O)Nc1ccc(C(F)(F)F)cc1F ; CCc1c(N2CCN(C(=O)C(=O)Nc3ccccn3)CC2)c(=O)n2nc(C3=CCOCC3)nc2n1CC(=O)Nc1ccc(C(F)(F)F)cc1F ; CCc1c(N2CCN(C(=O)CNC(=O)c3ccccc3)CC2)c(=O)n2nc(Br)nc2n1CC(=O)Nc1ccc(C(F)(F)F)cc1Cl] 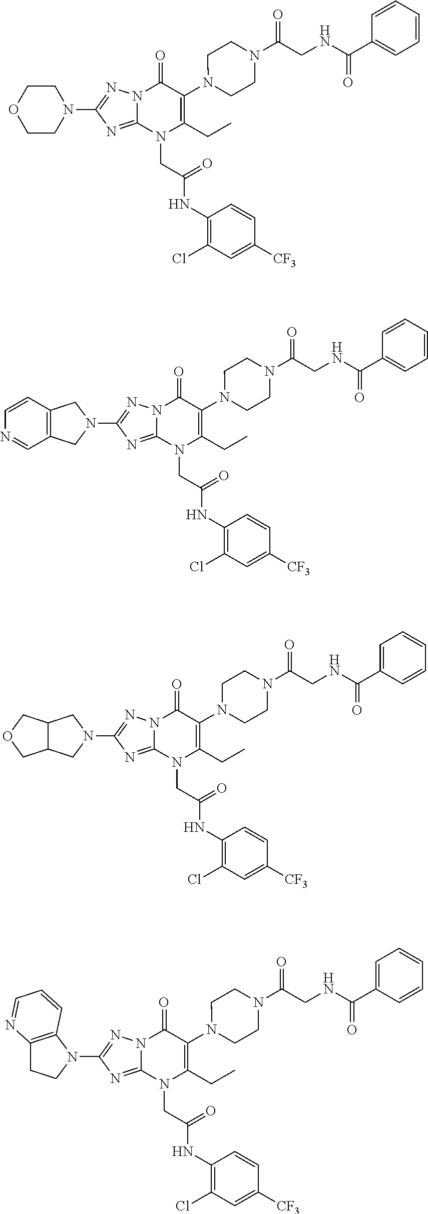 [SMILES: CCc1c(N2CCN(C(=O)CNC(=O)c3ccccc3)CC2)c(=O)n2nc(N3CCOCC3)nc2n1CC(=O)Nc1ccc(C(F)(F)F)cc1Cl ; CCc1c(N2CCN(C(=O)CNC(=O)c3ccccc3)CC2)c(=O)n2nc(N3Cc4ccncc4C3)nc2n1CC(=O)Nc1ccc(C(F)(F)F)cc1Cl ; CCc1c(N2CCN(C(=O)CNC(=O)c3ccccc3)CC2)c(=O)n2nc(N3CC4COCC4C3)nc2n1CC(=O)Nc1ccc(C(F)(F)F)cc1Cl ; CCc1c(N2CCN(C(=O)CNC(=O)c3ccccc3)CC2)c(=O)n2nc(N3CCc4ncccc43)nc2n1CC(=O)Nc1ccc(C(F)(F)F)cc1Cl] 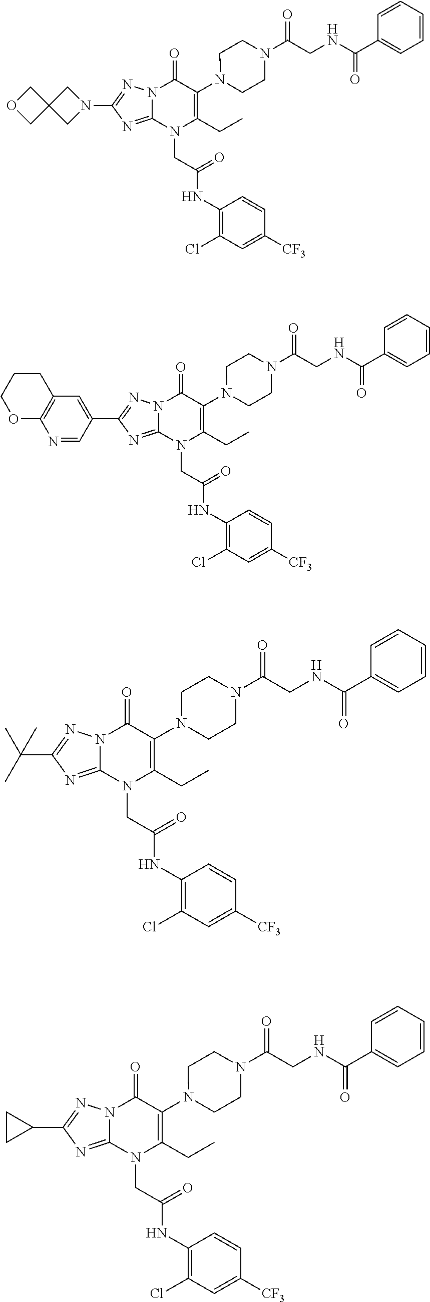 [SMILES: CCc1c(N2CCN(C(=O)CNC(=O)c3ccccc3)CC2)c(=O)n2nc(N3CC4(COC4)C3)nc2n1CC(=O)Nc1ccc(C(F)(F)F)cc1Cl ; CCc1c(N2CCN(C(=O)CNC(=O)c3ccccc3)CC2)c(=O)n2nc(-c3cnc4c(c3)CCCO4)nc2n1CC(=O)Nc1ccc(C(F)(F)F)cc1Cl ; CCc1c(N2CCN(C(=O)CNC(=O)c3ccccc3)CC2)c(=O)n2nc(C(C)(C)C)nc2n1CC(=O)Nc1ccc(C(F)(F)F)cc1Cl ; CCc1c(N2CCN(C(=O)CNC(=O)c3ccccc3)CC2)c(=O)n2nc(C3CC3)nc2n1CC(=O)Nc1ccc(C(F)(F)F)cc1Cl] 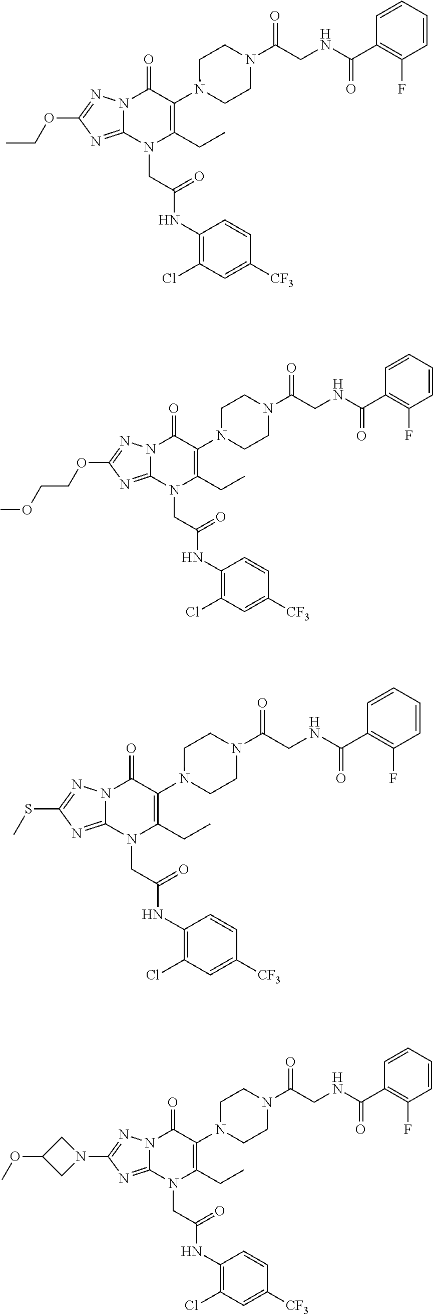 [SMILES: CCOc1nc2n(CC(=O)Nc3ccc(C(F)(F)F)cc3Cl)c(CC)c(N3CCN(C(=O)CNC(=O)c4ccccc4F)CC3)c(=O)n2n1 ; CCc1c(N2CCN(C(=O)CNC(=O)c3ccccc3F)CC2)c(=O)n2nc(OCCOC)nc2n1CC(=O)Nc1ccc(C(F)(F)F)cc1Cl ; CCc1c(N2CCN(C(=O)CNC(=O)c3ccccc3F)CC2)c(=O)n2nc(SC)nc2n1CC(=O)Nc1ccc(C(F)(F)F)cc1Cl ; CCc1c(N2CCN(C(=O)CNC(=O)c3ccccc3F)CC2)c(=O)n2nc(N3CC(OC)C3)nc2n1CC(=O)Nc1ccc(C(F)(F)F)cc1Cl] 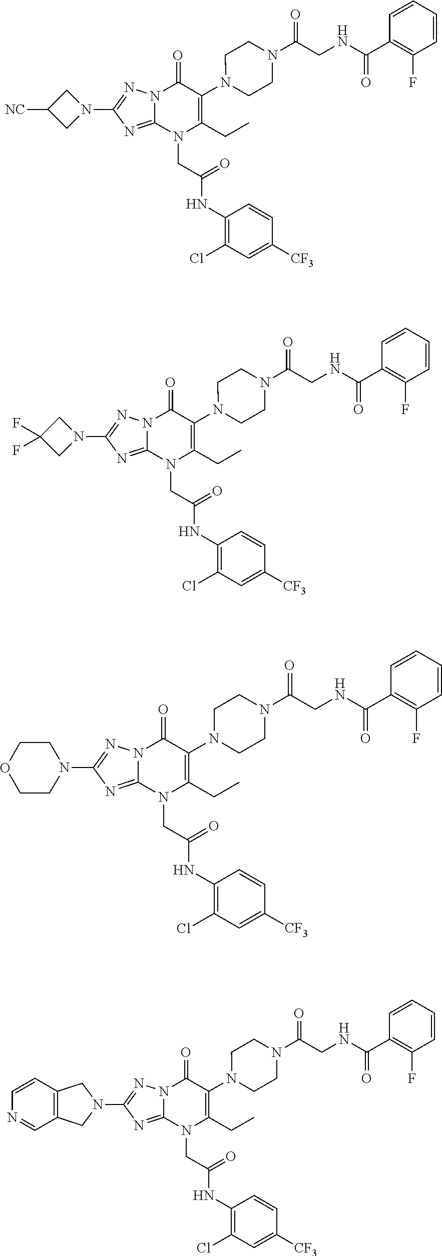 [SMILES: CCc1c(N2CCN(C(=O)CNC(=O)c3ccccc3F)CC2)c(=O)n2nc(N3CC(C#N)C3)nc2n1CC(=O)Nc1ccc(C(F)(F)F)cc1Cl ; CCc1c(N2CCN(C(=O)CNC(=O)c3ccccc3F)CC2)c(=O)n2nc(N3CC(F)(F)C3)nc2n1CC(=O)Nc1ccc(C(F)(F)F)cc1Cl ; CCc1c(N2CCN(C(=O)CNC(=O)c3ccccc3F)CC2)c(=O)n2nc(N3CCOCC3)nc2n1CC(=O)Nc1ccc(C(F)(F)F)cc1Cl ; CCc1c(N2CCN(C(=O)CNC(=O)c3ccccc3F)CC2)c(=O)n2nc(N3Cc4ccncc4C3)nc2n1CC(=O)Nc1ccc(C(F)(F)F)cc1Cl] 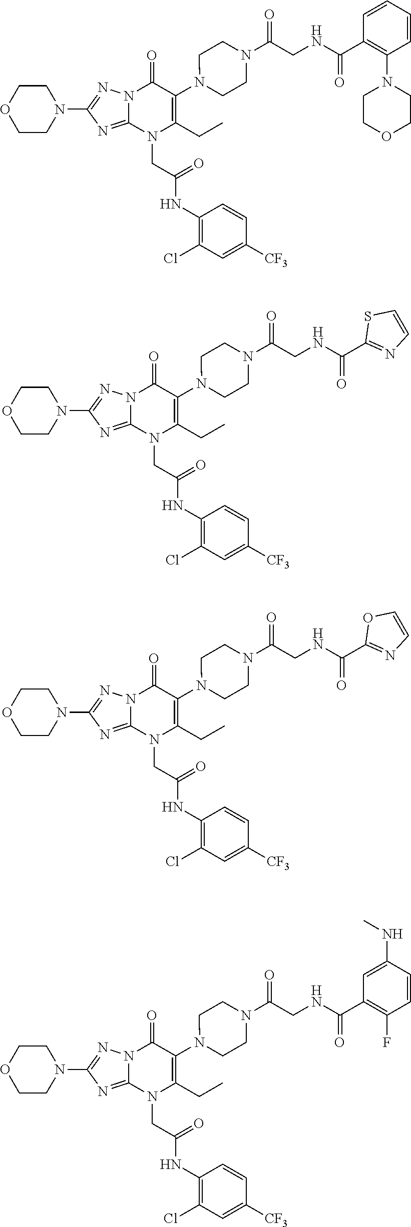 [SMILES: CCc1c(N2CCN(C(=O)CNC(=O)c3ccccc3N3CCOCC3)CC2)c(=O)n2nc(N3CCOCC3)nc2n1CC(=O)Nc1ccc(C(F)(F)F)cc1Cl ; CCc1c(N2CCN(C(=O)CNC(=O)c3nccs3)CC2)c(=O)n2nc(N3CCOCC3)nc2n1CC(=O)Nc1ccc(C(F)(F)F)cc1Cl ; CCc1c(N2CCN(C(=O)CNC(=O)c3ncco3)CC2)c(=O)n2nc(N3CCOCC3)nc2n1CC(=O)Nc1ccc(C(F)(F)F)cc1Cl ; CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1Cl] 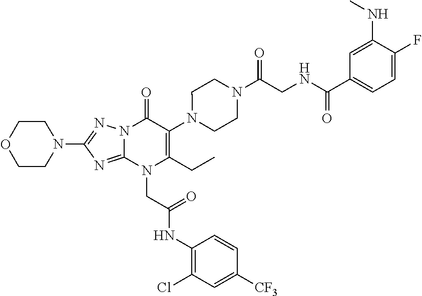 [SMILES: CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1Cl] 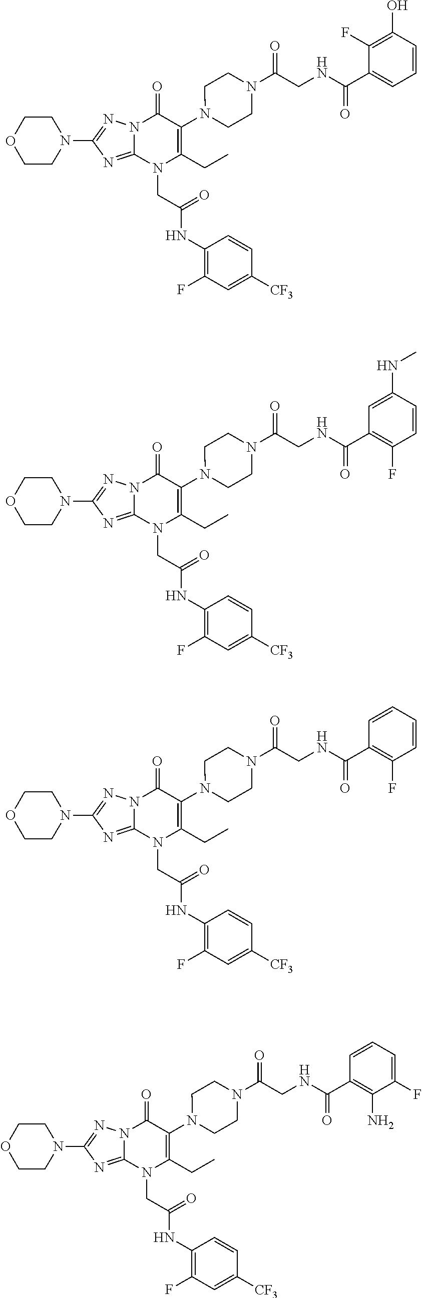 [SMILES: CCc1c(N2CCN(C(=O)CNC(=O)c3cccc(O)c3F)CC2)c(=O)n2nc(N3CCOCC3)nc2n1CC(=O)Nc1ccc(C(F)(F)F)cc1F ; CCc1c(N2CCN(C(=O)CNC(=O)c3cc(NC)ccc3F)CC2)c(=O)n2nc(N3CCOCC3)nc2n1CC(=O)Nc1ccc(C(F)(F)F)cc1F ; CCc1c(N2CCN(C(=O)CNC(=O)c3ccccc3F)CC2)c(=O)n2nc(N3CCOCC3)nc2n1CC(=O)Nc1ccc(C(F)(F)F)cc1F ; CCc1c(N2CCN(C(=O)CNC(=O)c3cccc(F)c3N)CC2)c(=O)n2nc(N3CCOCC3)nc2n1CC(=O)Nc1ccc(C(F)(F)F)cc1F] 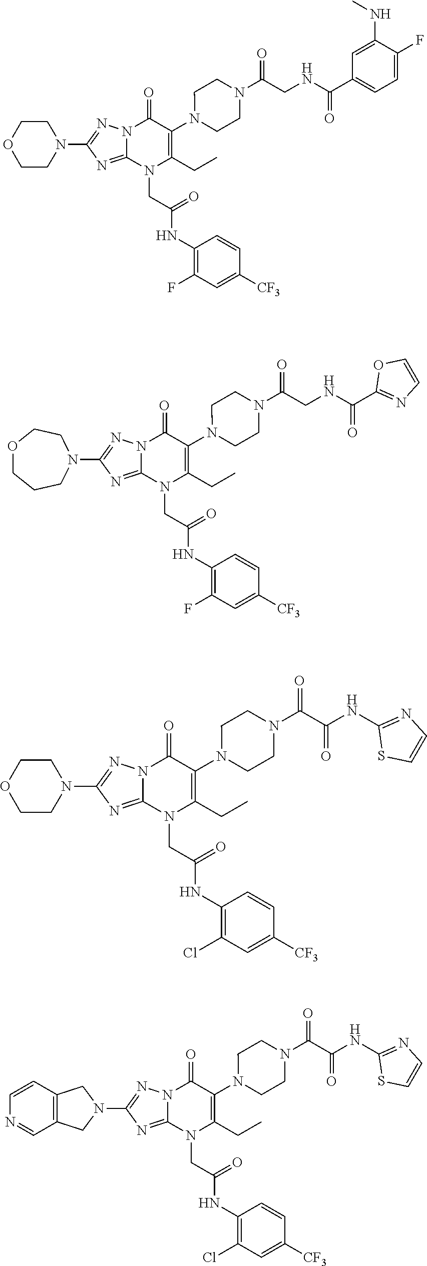 [SMILES: CCc1c(N2CCN(C(=O)CNC(=O)c3ccc(F)c(NC)c3)CC2)c(=O)n2nc(N3CCOCC3)nc2n1CC(=O)Nc1ccc(C(F)(F)F)cc1F ; CCc1c(N2CCN(C(=O)CNC(=O)c3ncco3)CC2)c(=O)n2nc(N3CCCOCC3)nc2n1CC(=O)Nc1ccc(C(F)(F)F)cc1F ; CCc1c(N2CCN(C(=O)C(=O)Nc3nccs3)CC2)c(=O)n2nc(N3CCOCC3)nc2n1CC(=O)Nc1ccc(C(F)(F)F)cc1Cl ; CCc1c(N2CCN(C(=O)C(=O)Nc3nccs3)CC2)c(=O)n2nc(N3Cc4ccncc4C3)nc2n1CC(=O)Nc1ccc(C(F)(F)F)cc1Cl] 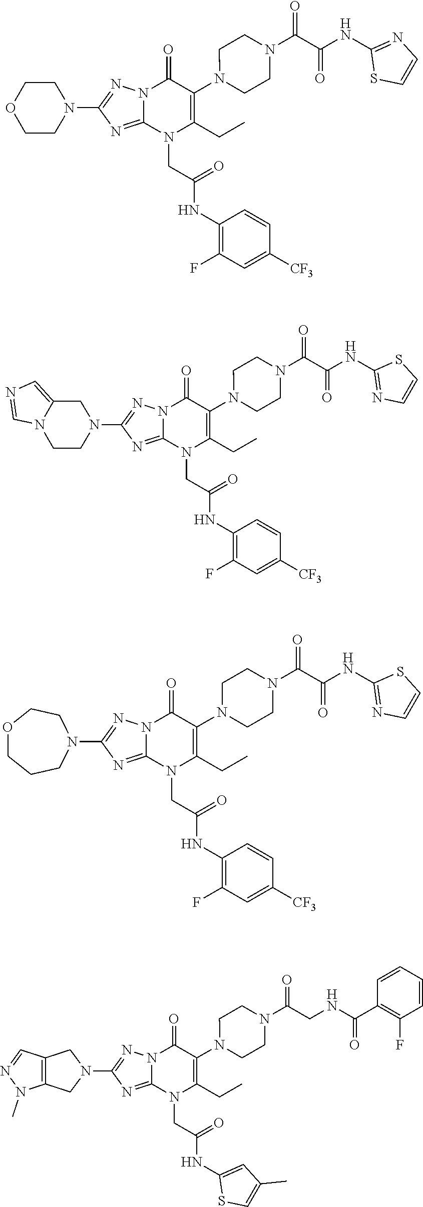 [SMILES: CCc1c(N2CCN(C(=O)C(=O)Nc3nccs3)CC2)c(=O)n2nc(N3CCOCC3)nc2n1CC(=O)Nc1ccc(C(F)(F)F)cc1F ; CCc1c(N2CCN(C(=O)C(=O)Nc3nccs3)CC2)c(=O)n2nc(N3CCn4cncc4C3)nc2n1CC(=O)Nc1ccc(C(F)(F)F)cc1F ; CCc1c(N2CCN(C(=O)C(=O)Nc3nccs3)CC2)c(=O)n2nc(N3CCCOCC3)nc2n1CC(=O)Nc1ccc(C(F)(F)F)cc1F ; CCc1c(N2CCN(C(=O)CNC(=O)c3ccccc3F)CC2)c(=O)n2nc(N3Cc4cnn(C)c4C3)nc2n1CC(=O)Nc1cc(C)cs1] 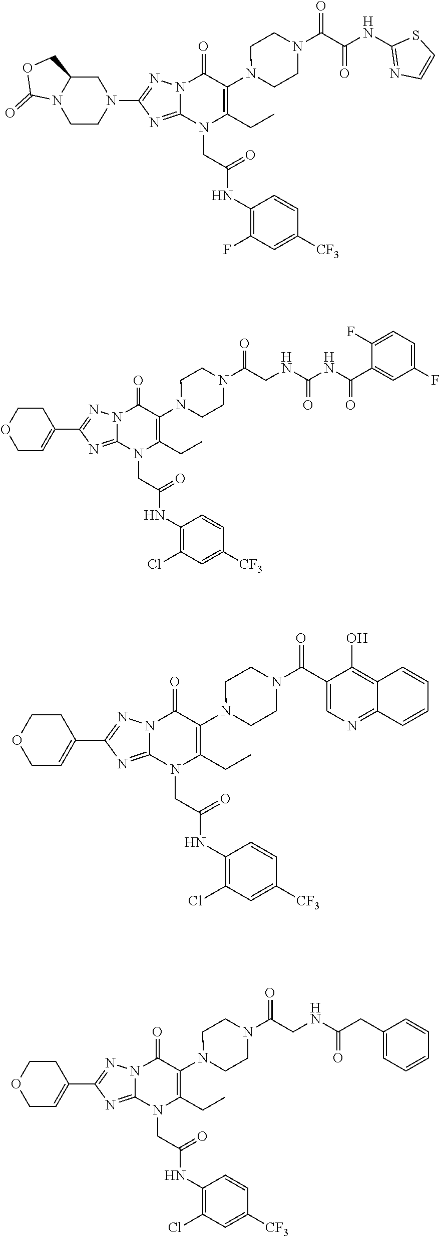 [SMILES: CCc1c(N2CCN(C(=O)C(=O)Nc3nccs3)CC2)c(=O)n2nc(N3CCN4C(=O)OC[C@H]4C3)nc2n1CC(=O)Nc1ccc(C(F)(F)F)cc1F ; CCc1c(N2CCN(C(=O)CNC(=O)NC(=O)c3cc(F)ccc3F)CC2)c(=O)n2nc(C3=CCOCC3)nc2n1CC(=O)Nc1ccc(C(F)(F)F)cc1Cl ; CCc1c(N2CCN(C(=O)c3cnc4ccccc4c3O)CC2)c(=O)n2nc(C3=CCOCC3)nc2n1CC(=O)Nc1ccc(C(F)(F)F)cc1Cl ; CCc1c(N2CCN(C(=O)CNC(=O)Cc3ccccc3)CC2)c(=O)n2nc(C3=CCOCC3)nc2n1CC(=O)Nc1ccc(C(F)(F)F)cc1Cl] 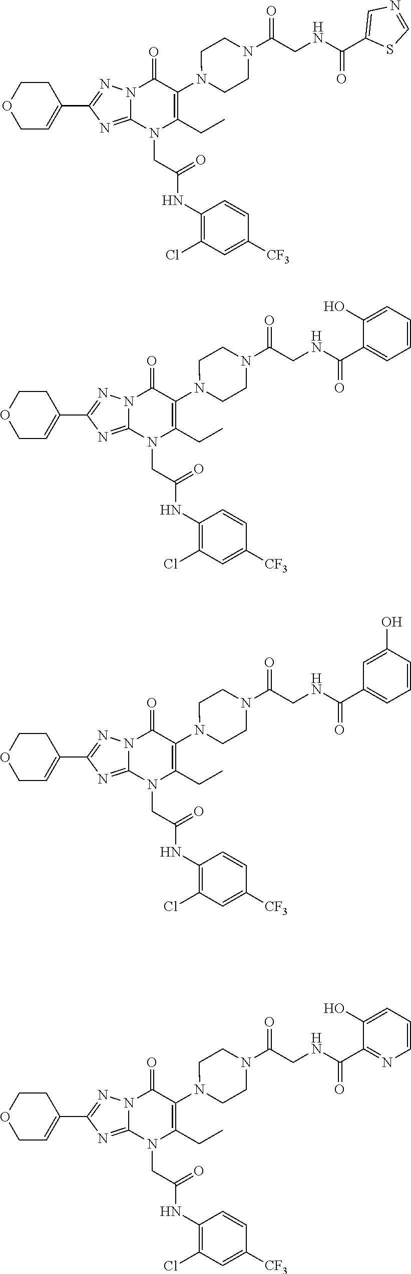 [SMILES: CCc1c(N2CCN(C(=O)CNC(=O)c3cncs3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ncccc3O)CC2)c(=O)n2nc(C3=CCOCC3)nc2n1CC(=O)Nc1ccc(C(F)(F)F)cc1Cl] 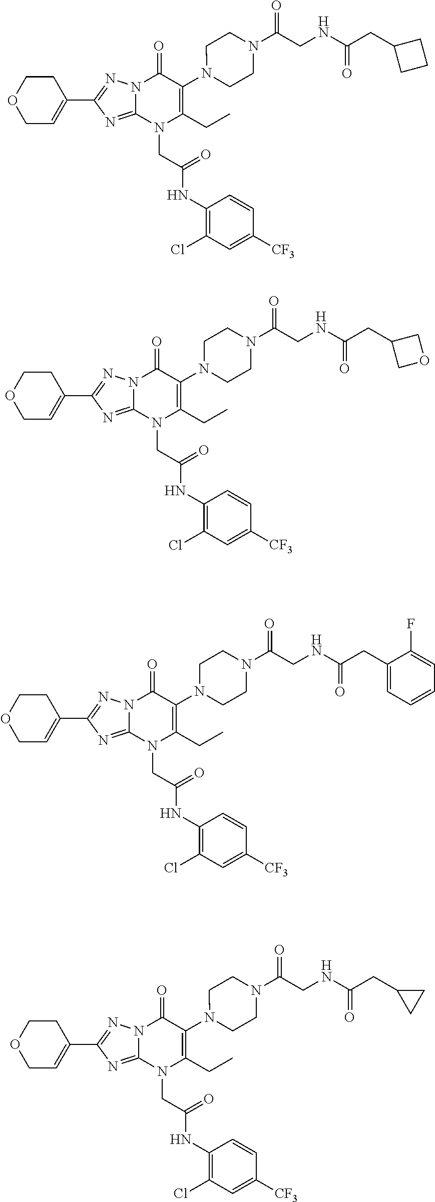 [SMILES: CCc1c(N2CCN(C(=O)CNC(=O)CC3CCC3)CC2)c(=O)n2nc(C3=CCOCC3)nc2n1CC(=O)Nc1ccc(C(F)(F)F)cc1Cl ; CCc1c(N2CCN(C(=O)CNC(=O)CC3COC3)CC2)c(=O)n2nc(C3=CCOCC3)nc2n1CC(=O)Nc1ccc(C(F)(F)F)cc1Cl ; CCc1c(N2CCN(C(=O)CNC(=O)Cc3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)CC3CC3)CC2)c(=O)n2nc(C3=CCOCC3)nc2n1CC(=O)Nc1ccc(C(F)(F)F)cc1Cl] 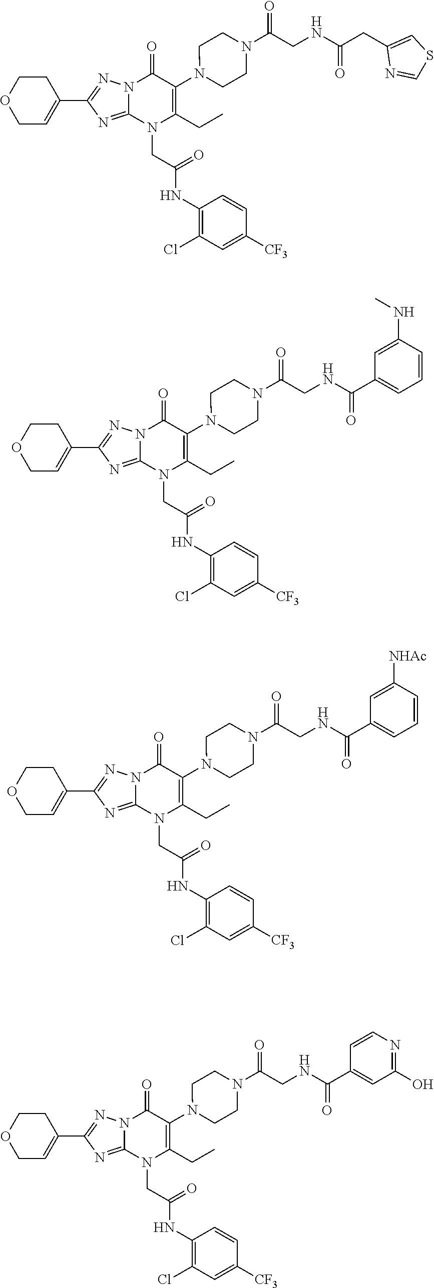 [SMILES: CCc1c(N2CCN(C(=O)CNC(=O)Cc3cscn3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC(C)=O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccnc(O)c3)CC2)c(=O)n2nc(C3=CCOCC3)nc2n1CC(=O)Nc1ccc(C(F)(F)F)cc1Cl] 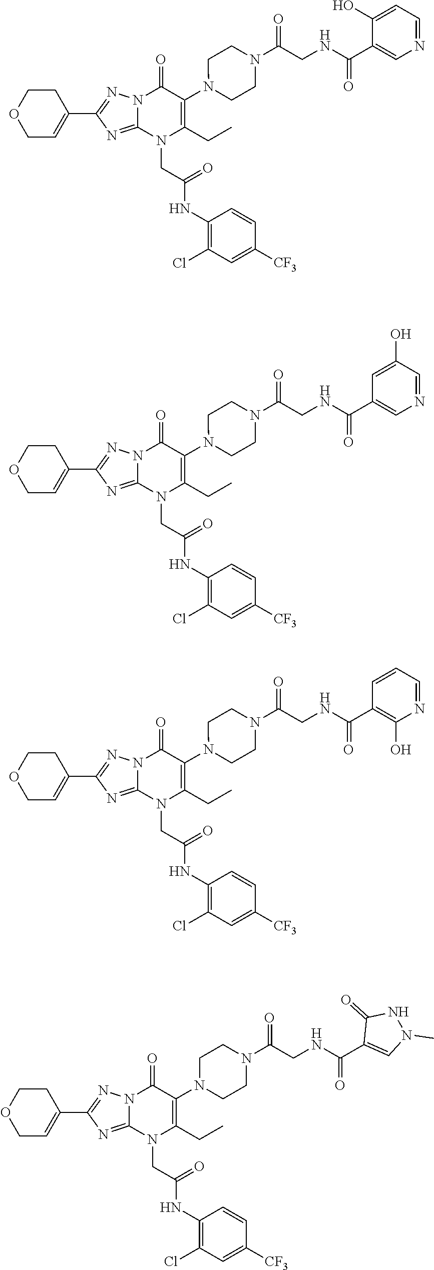 [SMILES: CCc1c(N2CCN(C(=O)CNC(=O)c3cnccc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cncc(O)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccnc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cn(C)[nH]c3=O)CC2)c(=O)n2nc(C3=CCOCC3)nc2n1CC(=O)Nc1ccc(C(F)(F)F)cc1Cl] 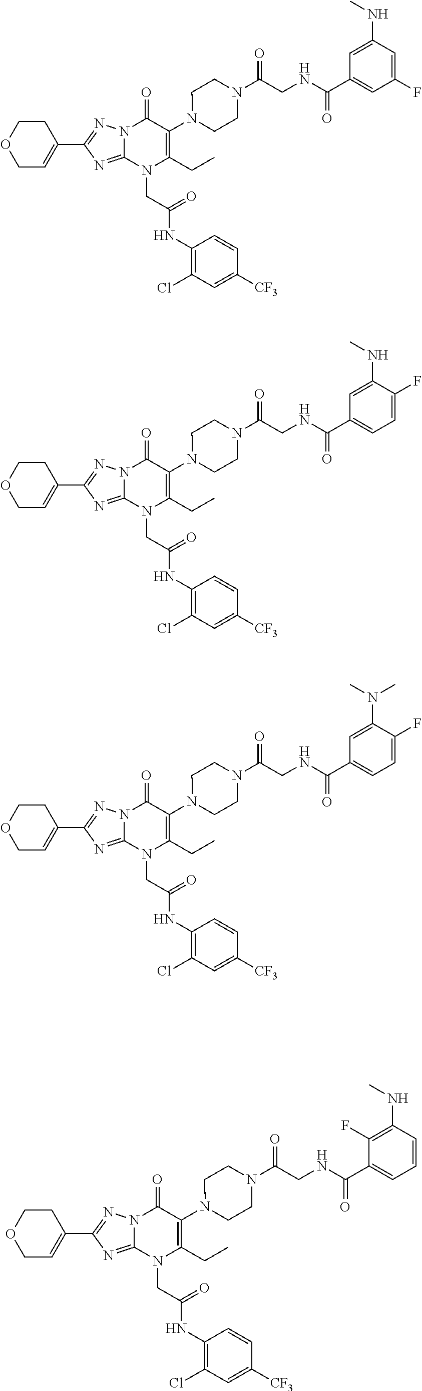 [SMILES: CCc1c(N2CCN(C(=O)CNC(=O)c3cc(F)cc(NC)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)c(NC)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)c(N(C)C)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(NC)c3F)CC2)c(=O)n2nc(C3=CCOCC3)nc2n1CC(=O)Nc1ccc(C(F)(F)F)cc1Cl] 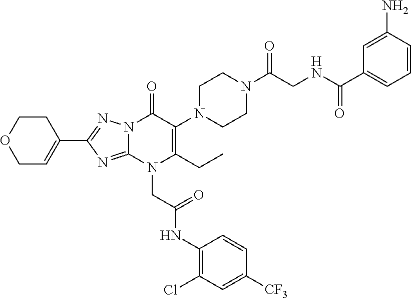 [SMILES: CCc1c(N2CCN(C(=O)CNC(=O)c3cccc(N)c3)CC2)c(=O)n2nc(C3=CCOCC3)nc2n1CC(=O)Nc1ccc(C(F)(F)F)cc1Cl] 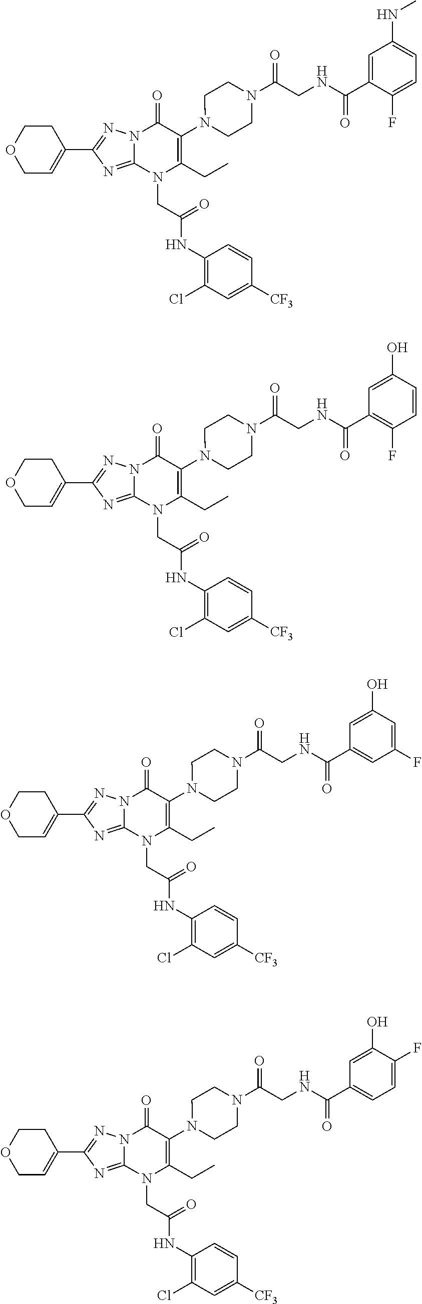 [SMILES: CCc1c(N2CCN(C(=O)CNC(=O)c3cc(NC)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(O)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(O)cc(F)c3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(F)c(O)c3)CC2)c(=O)n2nc(C3=CCOCC3)nc2n1CC(=O)Nc1ccc(C(F)(F)F)cc1Cl] 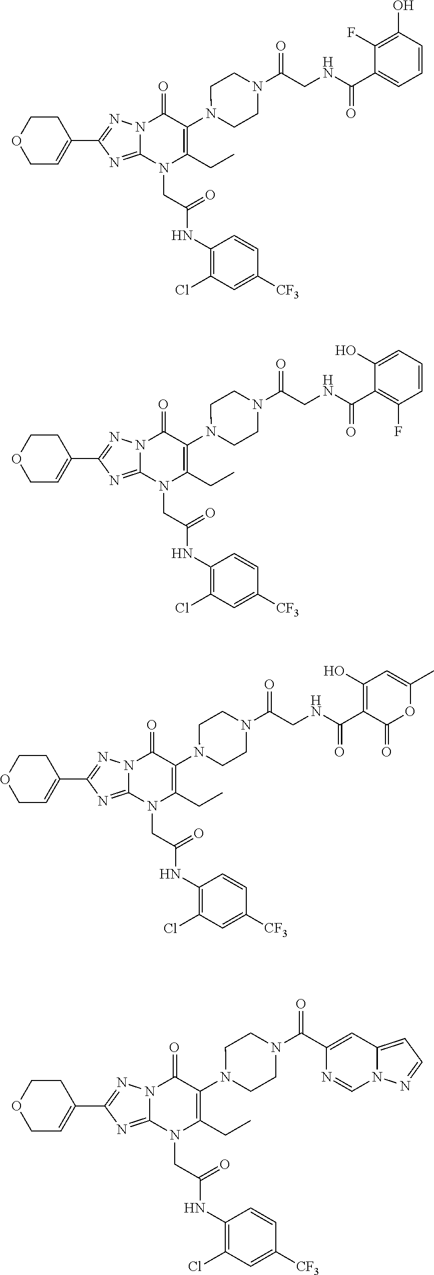 [SMILES: CCc1c(N2CCN(C(=O)CNC(=O)c3cccc(O)c3F)CC2)c(=O)n2nc(C3=CCOCC3)nc2n1CC(=O)Nc1ccc(C(F)(F)F)cc1Cl ; CCc1c(N2CCN(C(=O)CNC(=O)c3c(O)cccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3c(O)cc(C)oc3=O)CC2)c(=O)n2nc(C3=CCOCC3)nc2n1CC(=O)Nc1ccc(C(F)(F)F)cc1Cl ; CCc1c(N2CCN(C(=O)c3cc4ccnn4cn3)CC2)c(=O)n2nc(C3=CCOCC3)nc2n1CC(=O)Nc1ccc(C(F)(F)F)cc1Cl] 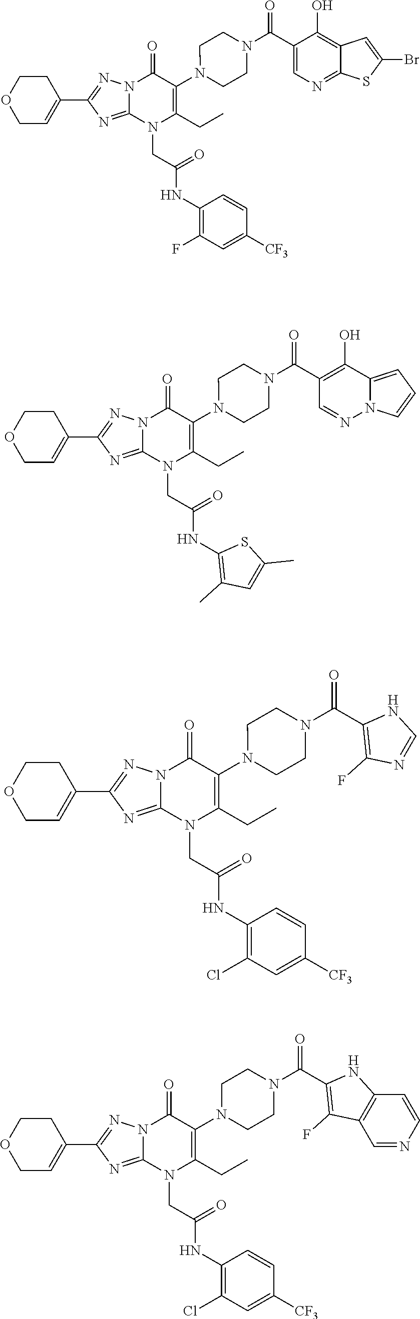 [SMILES: CCc1c(N2CCN(C(=O)c3cnc4sc(Br)cc4c3O)CC2)c(=O)n2nc(C3=CCOCC3)nc2n1CC(=O)Nc1ccc(C(F)(F)F)cc1F ; CCc1c(N2CCN(C(=O)c3cnn4cccc4c3O)CC2)c(=O)n2nc(C3=CCOCC3)nc2n1CC(=O)Nc1sc(C)cc1C ; CCc1c(N2CCN(C(=O)c3[nH]cnc3F)CC2)c(=O)n2nc(C3=CCOCC3)nc2n1CC(=O)Nc1ccc(C(F)(F)F)cc1Cl ; CCc1c(N2CCN(C(=O)c3[nH]c4ccncc4c3F)CC2)c(=O)n2nc(C3=CCOCC3)nc2n1CC(=O)Nc1ccc(C(F)(F)F)cc1Cl] 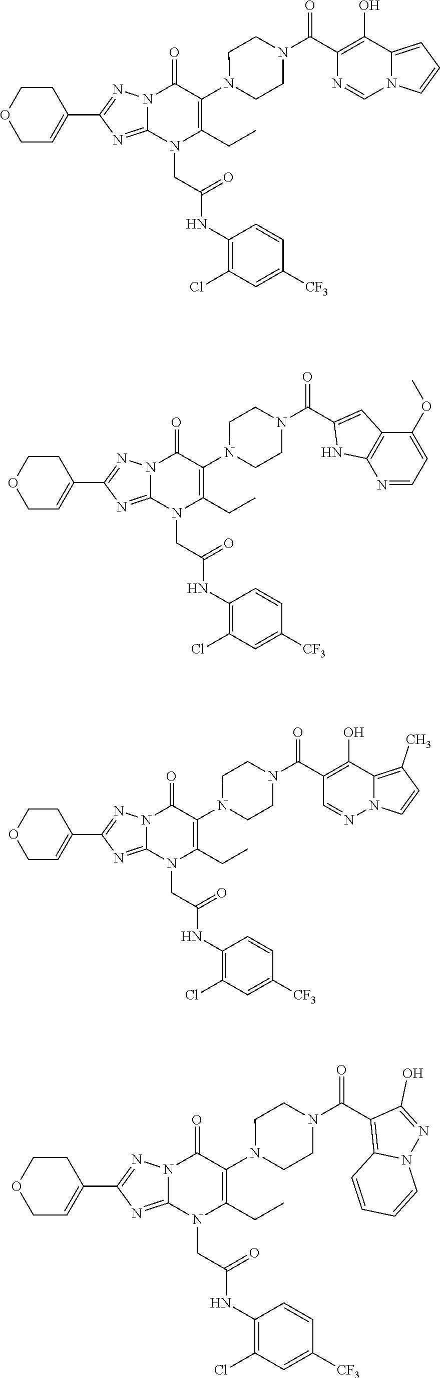 [SMILES: CCc1c(N2CCN(C(=O)c3ncn4cccc4c3O)CC2)c(=O)n2nc(C3=CCOCC3)nc2n1CC(=O)Nc1ccc(C(F)(F)F)cc1Cl ; CCc1c(N2CCN(C(=O)c3cc4c(OC)ccnc4[nH]3)CC2)c(=O)n2nc(C3=CCOCC3)nc2n1CC(=O)Nc1ccc(C(F)(F)F)cc1Cl ; CCc1c(N2CCN(C(=O)c3cnn4ccc(C)c4c3O)CC2)c(=O)n2nc(C3=CCOCC3)nc2n1CC(=O)Nc1ccc(C(F)(F)F)cc1Cl ; CCc1c(N2CCN(C(=O)c3c(O)nn4ccccc34)CC2)c(=O)n2nc(C3=CCOCC3)nc2n1CC(=O)Nc1ccc(C(F)(F)F)cc1Cl] 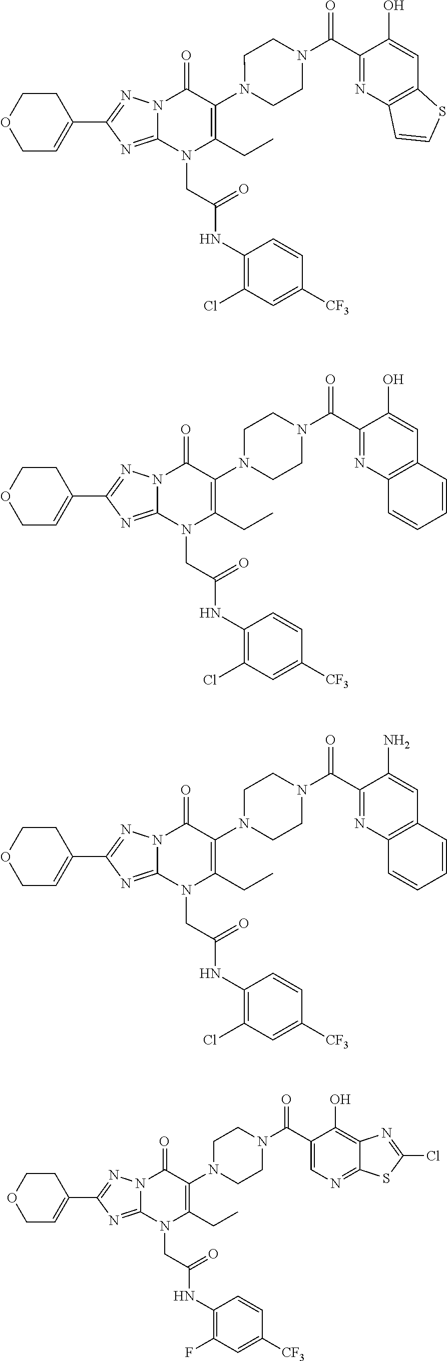 [SMILES: CCc1c(N2CCN(C(=O)c3nc4ccsc4cc3O)CC2)c(=O)n2nc(C3=CCOCC3)nc2n1CC(=O)Nc1ccc(C(F)(F)F)cc1Cl ; CCc1c(N2CCN(C(=O)c3nc4ccccc4cc3O)CC2)c(=O)n2nc(C3=CCOCC3)nc2n1CC(=O)Nc1ccc(C(F)(F)F)cc1Cl ; CCc1c(N2CCN(C(=O)c3nc4ccccc4cc3N)CC2)c(=O)n2nc(C3=CCOCC3)nc2n1CC(=O)Nc1ccc(C(F)(F)F)cc1Cl ; CCc1c(N2CCN(C(=O)c3cnc4sc(Cl)nc4c3O)CC2)c(=O)n2nc(C3=CCOCC3)nc2n1CC(=O)Nc1ccc(C(F)(F)F)cc1F] 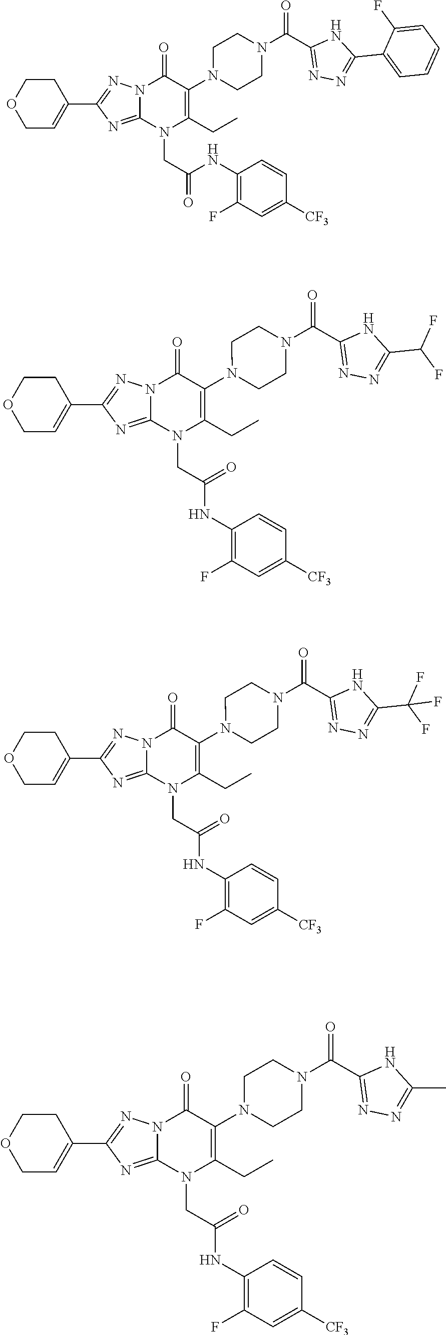 [SMILES: CCc1c(N2CCN(C(=O)c3nnc(-c4ccccc4F)[nH]3)CC2)c(=O)n2nc(C3=CCOCC3)nc2n1CC(=O)Nc1ccc(C(F)(F)F)cc1F ; CCc1c(N2CCN(C(=O)c3nnc(C(F)F)[nH]3)CC2)c(=O)n2nc(C3=CCOCC3)nc2n1CC(=O)Nc1ccc(C(F)(F)F)cc1F ; CCc1c(N2CCN(C(=O)c3nnc(C(F)(F)F)[nH]3)CC2)c(=O)n2nc(C3=CCOCC3)nc2n1CC(=O)Nc1ccc(C(F)(F)F)cc1F ; CCc1c(N2CCN(C(=O)c3nnc(C)[nH]3)CC2)c(=O)n2nc(C3=CCOCC3)nc2n1CC(=O)Nc1ccc(C(F)(F)F)cc1F] 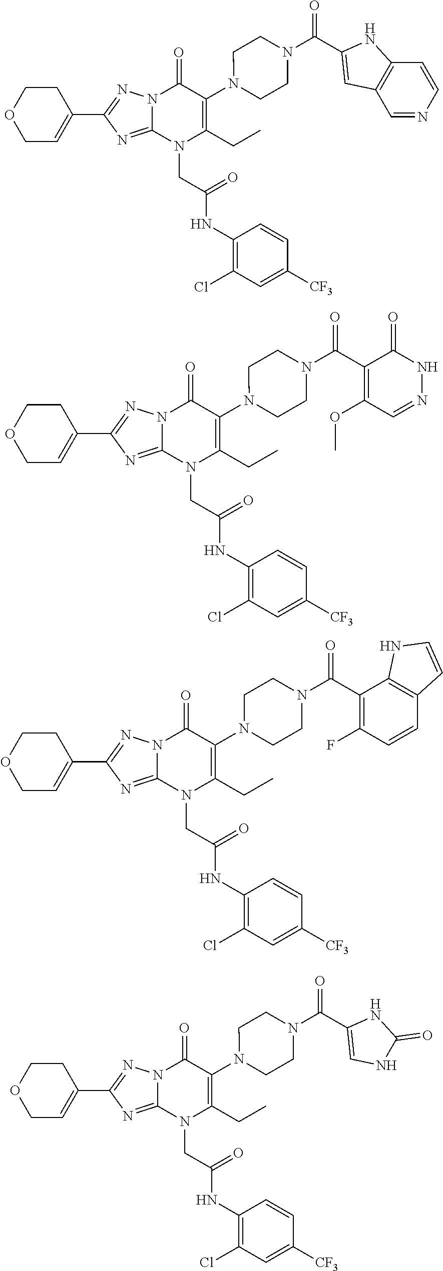 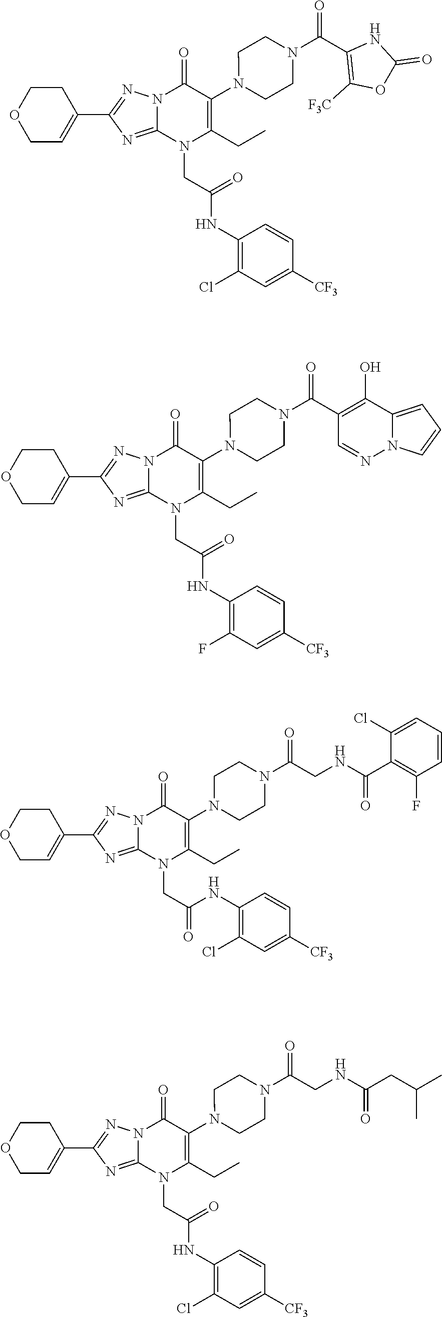 [SMILES: CCc1c(N2CCN(C(=O)c3[nH]c(=O)oc3C(F)(F)F)CC2)c(=O)n2nc(C3=CCOCC3)nc2n1CC(=O)Nc1ccc(C(F)(F)F)cc1Cl ; CCc1c(N2CCN(C(=O)c3cnn4cccc4c3O)CC2)c(=O)n2nc(C3=CCOCC3)nc2n1CC(=O)Nc1ccc(C(F)(F)F)cc1F ; CCc1c(N2CCN(C(=O)CNC(=O)c3c(F)cccc3Cl)CC2)c(=O)n2nc(C3=CCOCC3)nc2n1CC(=O)Nc1ccc(C(F)(F)F)cc1Cl ; CCc1c(N2CCN(C(=O)CNC(=O)CC(C)C)CC2)c(=O)n2nc(C3=CCOCC3)nc2n1CC(=O)Nc1ccc(C(F)(F)F)cc1Cl] 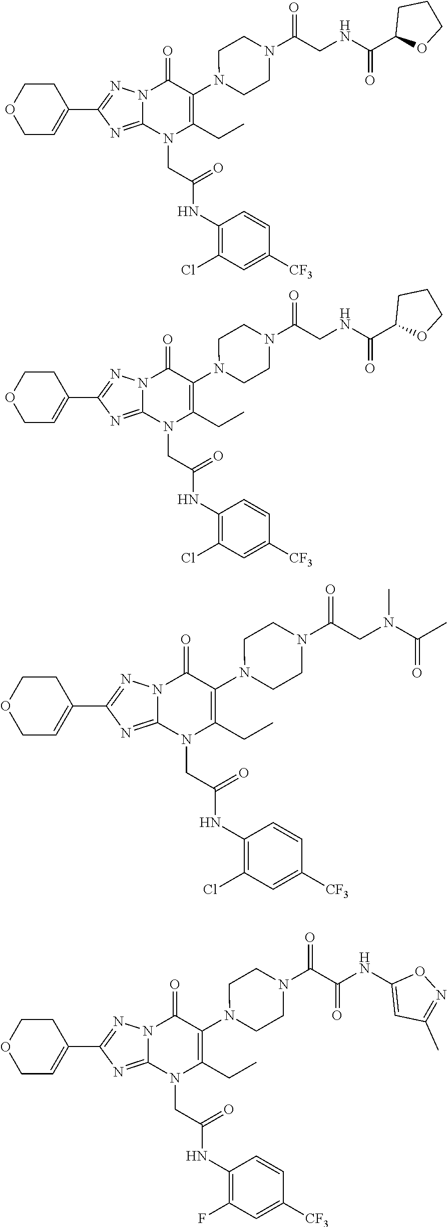 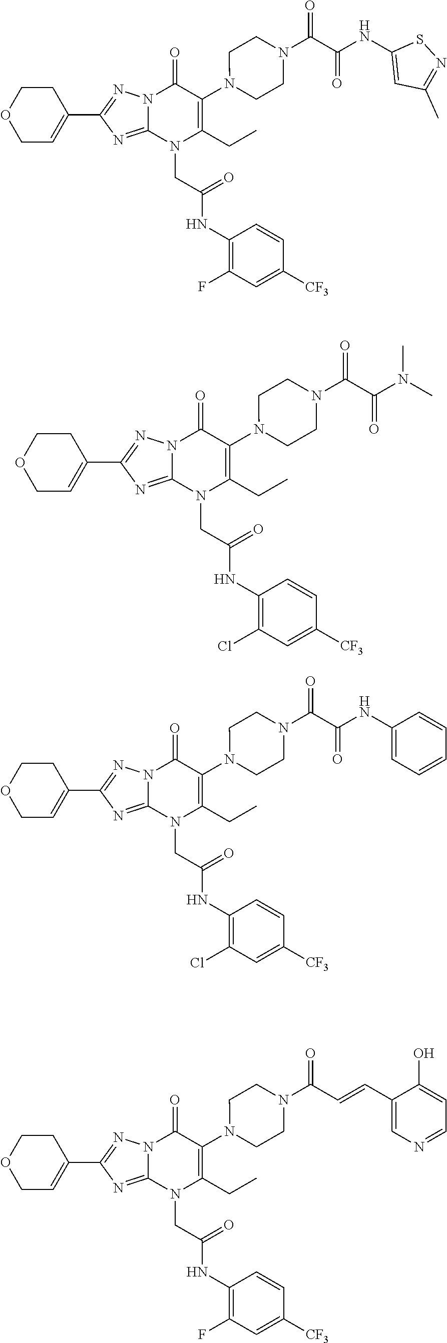 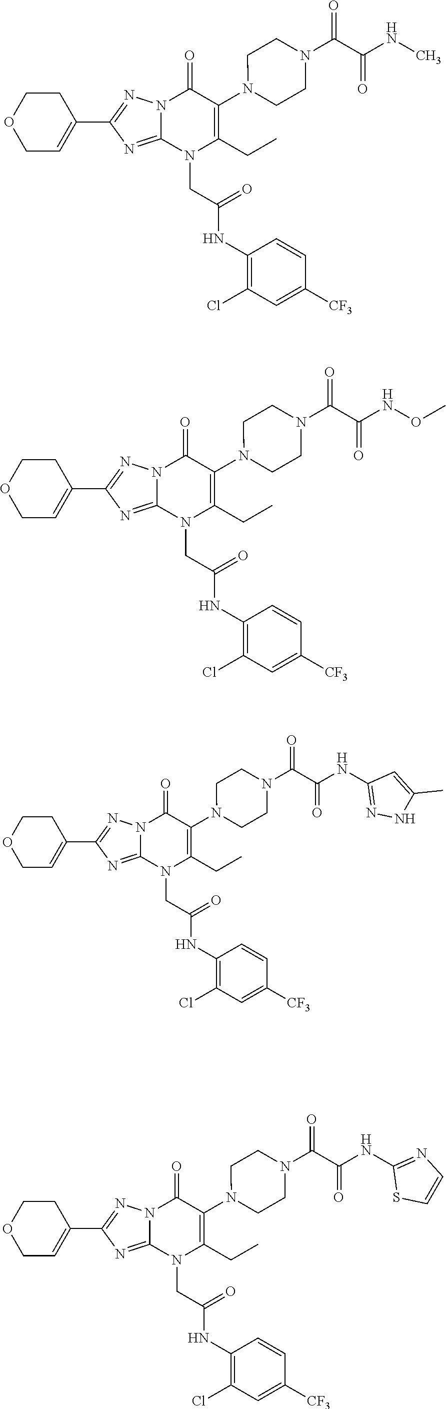 [SMILES: CCc1c(N2CCN(C(=O)C(=O)NC)CC2)c(=O)n2nc(C3=CCOCC3)nc2n1CC(=O)Nc1ccc(C(F)(F)F)cc1Cl ; CCc1c(N2CCN(C(=O)C(=O)NOC)CC2)c(=O)n2nc(C3=CCOCC3)nc2n1CC(=O)Nc1ccc(C(F)(F)F)cc1Cl ; CCc1c(N2CCN(C(=O)C(=O)Nc3cc(C)[nH]n3)CC2)c(=O)n2nc(C3=CCOCC3)nc2n1CC(=O)Nc1ccc(C(F)(F)F)cc1Cl ; CCc1c(N2CCN(C(=O)C(=O)Nc3nccs3)CC2)c(=O)n2nc(C3=CCOCC3)nc2n1CC(=O)Nc1ccc(C(F)(F)F)cc1Cl] 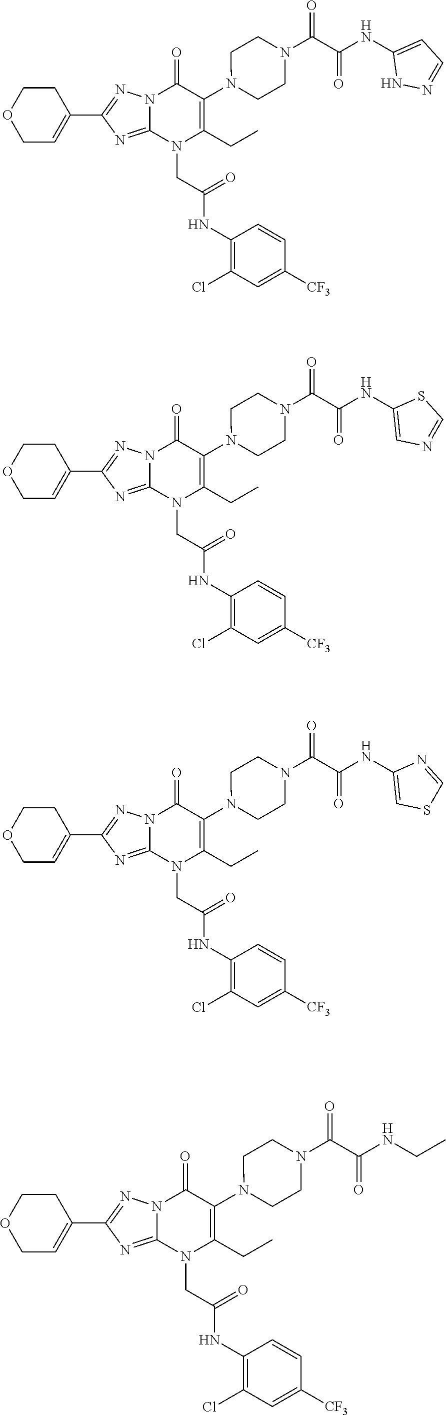 [SMILES: CCc1c(N2CCN(C(=O)C(=O)Nc3ccn[nH]3)CC2)c(=O)n2nc(C3=CCOCC3)nc2n1CC(=O)Nc1ccc(C(F)(F)F)cc1Cl ; CCc1c(N2CCN(C(=O)C(=O)Nc3cncs3)CC2)c(=O)n2nc(C3=CCOCC3)nc2n1CC(=O)Nc1ccc(C(F)(F)F)cc1Cl ; CCc1c(N2CCN(C(=O)C(=O)Nc3cscn3)CC2)c(=O)n2nc(C3=CCOCC3)nc2n1CC(=O)Nc1ccc(C(F)(F)F)cc1Cl ; CCNC(=O)C(=O)N1CCN(c2c(CC)n(CC(=O)Nc3ccc(C(F)(F)F)cc3Cl)c3nc(C4=CCOCC4)nn3c2=O)CC1] 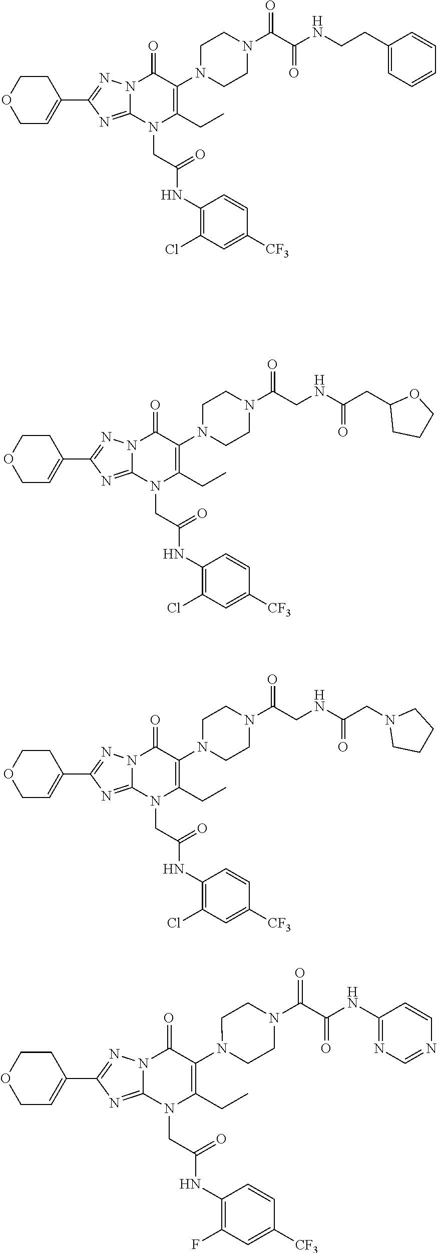 [SMILES: CCc1c(N2CCN(C(=O)C(=O)NCCc3ccccc3)CC2)c(=O)n2nc(C3=CCOCC3)nc2n1CC(=O)Nc1ccc(C(F)(F)F)cc1Cl ; CCc1c(N2CCN(C(=O)CNC(=O)CC3CCCO3)CC2)c(=O)n2nc(C3=CCOCC3)nc2n1CC(=O)Nc1ccc(C(F)(F)F)cc1Cl ; CCc1c(N2CCN(C(=O)CNC(=O)CN3CCCC3)CC2)c(=O)n2nc(C3=CCOCC3)nc2n1CC(=O)Nc1ccc(C(F)(F)F)cc1Cl ; CCc1c(N2CCN(C(=O)C(=O)Nc3ccncn3)CC2)c(=O)n2nc(C3=CCOCC3)nc2n1CC(=O)Nc1ccc(C(F)(F)F)cc1F] 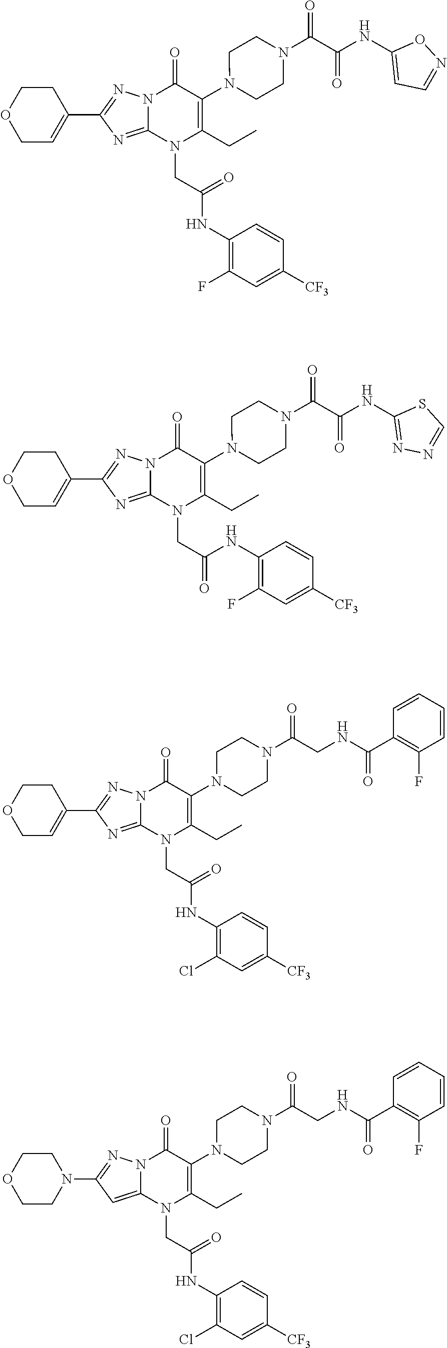 [SMILES: CCc1c(N2CCN(C(=O)C(=O)Nc3ccno3)CC2)c(=O)n2nc(C3=CCOCC3)nc2n1CC(=O)Nc1ccc(C(F)(F)F)cc1F ; CCc1c(N2CCN(C(=O)C(=O)Nc3nncs3)CC2)c(=O)n2nc(C3=CCOCC3)nc2n1CC(=O)Nc1ccc(C(F)(F)F)cc1F ; CCc1c(N2CCN(C(=O)CNC(=O)c3ccccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccccc3F)CC2)c(=O)n2nc(N3CCOCC3)cc2n1CC(=O)Nc1ccc(C(F)(F)F)cc1Cl] 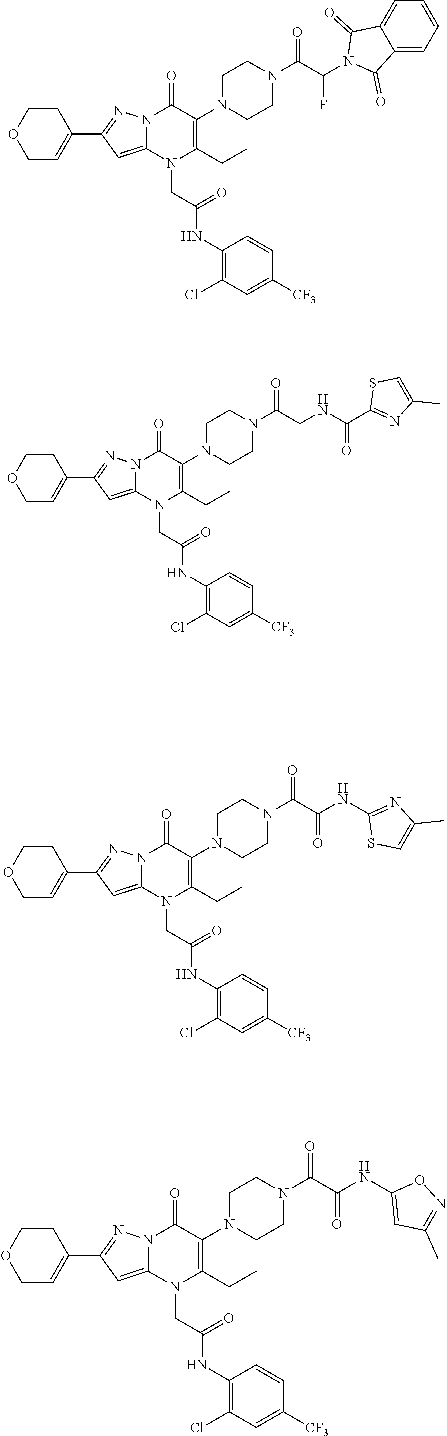 [SMILES: CCc1c(N2CCN(C(=O)C(F)N3C(=O)c4ccccc4C3=O)CC2)c(=O)n2nc(C3=CCOCC3)cc2n1CC(=O)Nc1ccc(C(F)(F)F)cc1Cl ; CCc1c(N2CCN(C(=O)CNC(=O)c3nc(C)cs3)CC2)c(=O)n2nc(C3=CCOCC3)cc2n1CC(=O)Nc1ccc(C(F)(F)F)cc1Cl ; CCc1c(N2CCN(C(=O)C(=O)Nc3nc(C)cs3)CC2)c(=O)n2nc(C3=CCOCC3)cc2n1CC(=O)Nc1ccc(C(F)(F)F)cc1Cl ; CCc1c(N2CCN(C(=O)C(=O)Nc3cc(C)no3)CC2)c(=O)n2nc(C3=CCOCC3)cc2n1CC(=O)Nc1ccc(C(F)(F)F)cc1Cl] 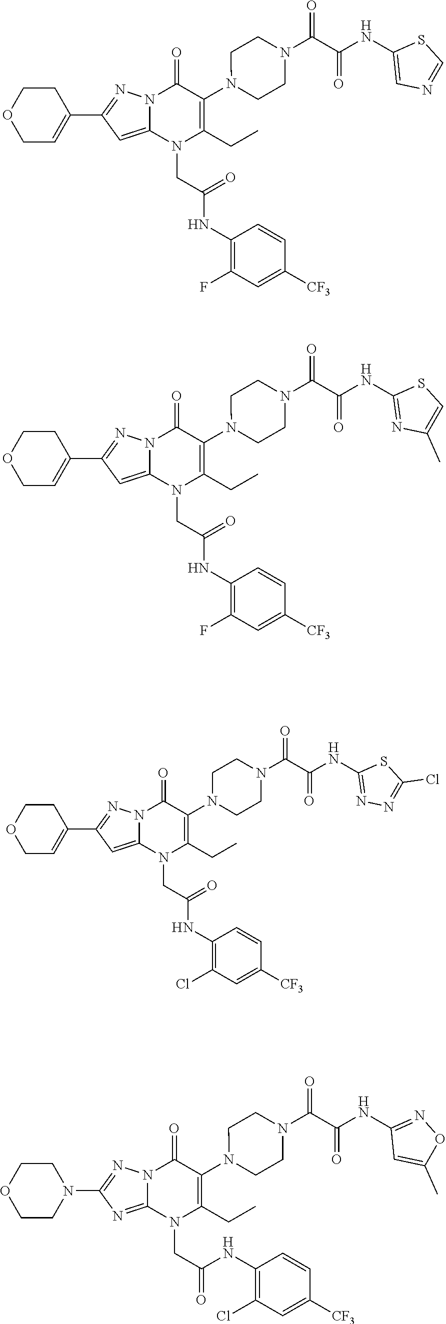 [SMILES: CCc1c(N2CCN(C(=O)C(=O)Nc3cncs3)CC2)c(=O)n2nc(C3=CCOCC3)cc2n1CC(=O)Nc1ccc(C(F)(F)F)cc1F ; CCc1c(N2CCN(C(=O)C(=O)Nc3nc(C)cs3)CC2)c(=O)n2nc(C3=CCOCC3)cc2n1CC(=O)Nc1ccc(C(F)(F)F)cc1F ; CCc1c(N2CCN(C(=O)C(=O)Nc3nnc(Cl)s3)CC2)c(=O)n2nc(C3=CCOCC3)cc2n1CC(=O)Nc1ccc(C(F)(F)F)cc1Cl ; CCc1c(N2CCN(C(=O)C(=O)Nc3cc(C)on3)CC2)c(=O)n2nc(N3CCOCC3)nc2n1CC(=O)Nc1ccc(C(F)(F)F)cc1Cl] 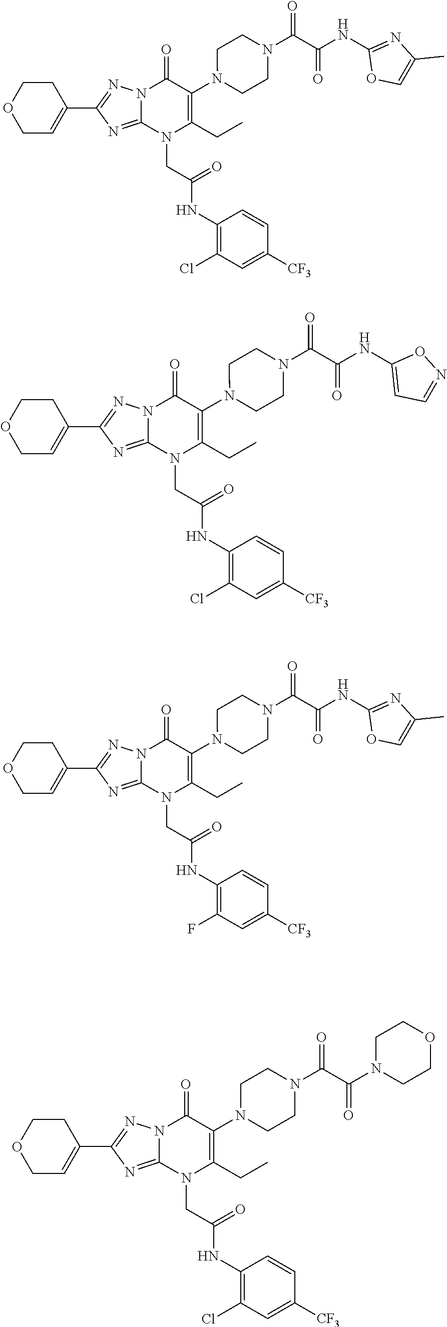 [SMILES: CCc1c(N2CCN(C(=O)C(=O)Nc3nc(C)co3)CC2)c(=O)n2nc(C3=CCOCC3)nc2n1CC(=O)Nc1ccc(C(F)(F)F)cc1Cl ; CCc1c(N2CCN(C(=O)C(=O)Nc3ccno3)CC2)c(=O)n2nc(C3=CCOCC3)nc2n1CC(=O)Nc1ccc(C(F)(F)F)cc1Cl ; CCc1c(N2CCN(C(=O)C(=O)Nc3nc(C)co3)CC2)c(=O)n2nc(C3=CCOCC3)nc2n1CC(=O)Nc1ccc(C(F)(F)F)cc1F ; CCc1c(N2CCN(C(=O)C(=O)N3CCOCC3)CC2)c(=O)n2nc(C3=CCOCC3)nc2n1CC(=O)Nc1ccc(C(F)(F)F)cc1Cl] 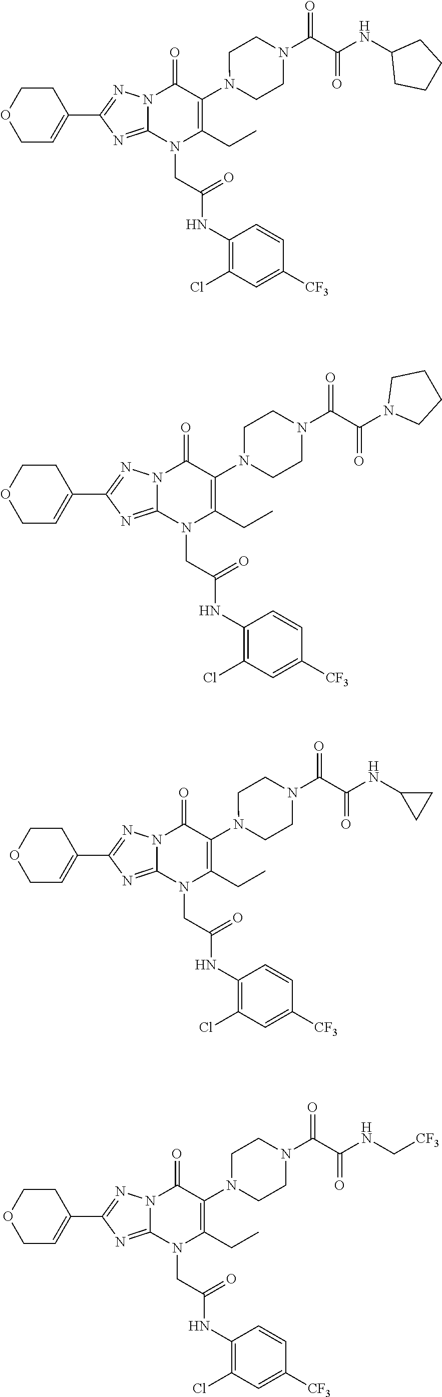 [SMILES: CCc1c(N2CCN(C(=O)C(=O)NC3CCCC3)CC2)c(=O)n2nc(C3=CCOCC3)nc2n1CC(=O)Nc1ccc(C(F)(F)F)cc1Cl ; CCc1c(N2CCN(C(=O)C(=O)N3CCCC3)CC2)c(=O)n2nc(C3=CCOCC3)nc2n1CC(=O)Nc1ccc(C(F)(F)F)cc1Cl ; CCc1c(N2CCN(C(=O)C(=O)NC3CC3)CC2)c(=O)n2nc(C3=CCOCC3)nc2n1CC(=O)Nc1ccc(C(F)(F)F)cc1Cl ; CCc1c(N2CCN(C(=O)C(=O)NCC(F)(F)F)CC2)c(=O)n2nc(C3=CCOCC3)nc2n1CC(=O)Nc1ccc(C(F)(F)F)cc1Cl] 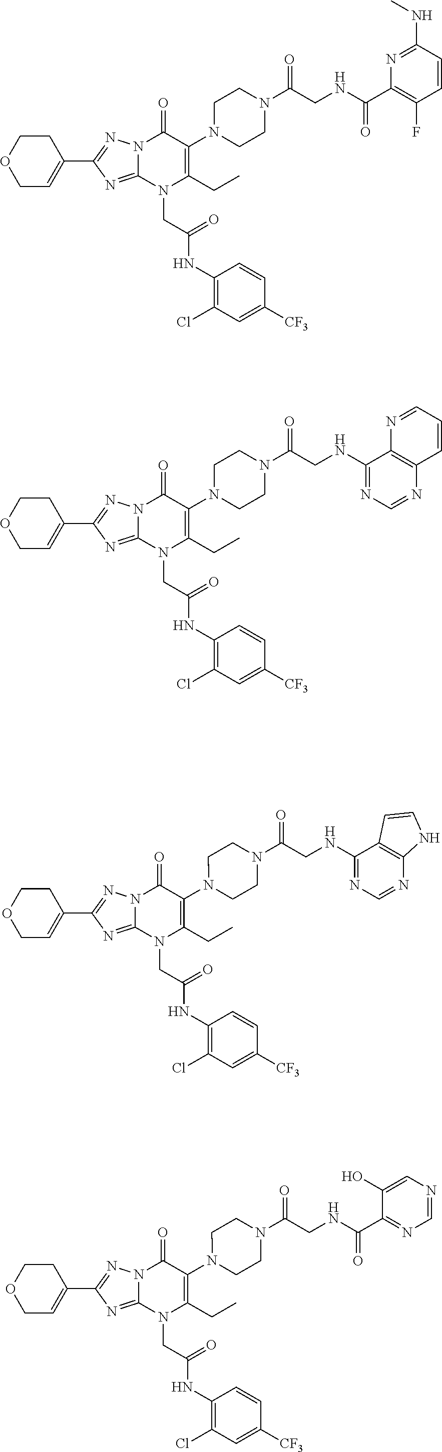 [SMILES: CCc1c(N2CCN(C(=O)CNC(=O)c3nc(NC)ccc3F)CC2)c(=O)n2nc(C3=CCOCC3)nc2n1CC(=O)Nc1ccc(C(F)(F)F)cc1Cl ; CCc1c(N2CCN(C(=O)CNc3ncnc4cccnc34)CC2)c(=O)n2nc(C3=CCOCC3)nc2n1CC(=O)Nc1ccc(C(F)(F)F)cc1Cl ; CCc1c(N2CCN(C(=O)CNc3ncnc4[nH]ccc34)CC2)c(=O)n2nc(C3=CCOCC3)nc2n1CC(=O)Nc1ccc(C(F)(F)F)cc1Cl ; CCc1c(N2CCN(C(=O)CNC(=O)c3ncncc3O)CC2)c(=O)n2nc(C3=CCOCC3)nc2n1CC(=O)Nc1ccc(C(F)(F)F)cc1Cl] 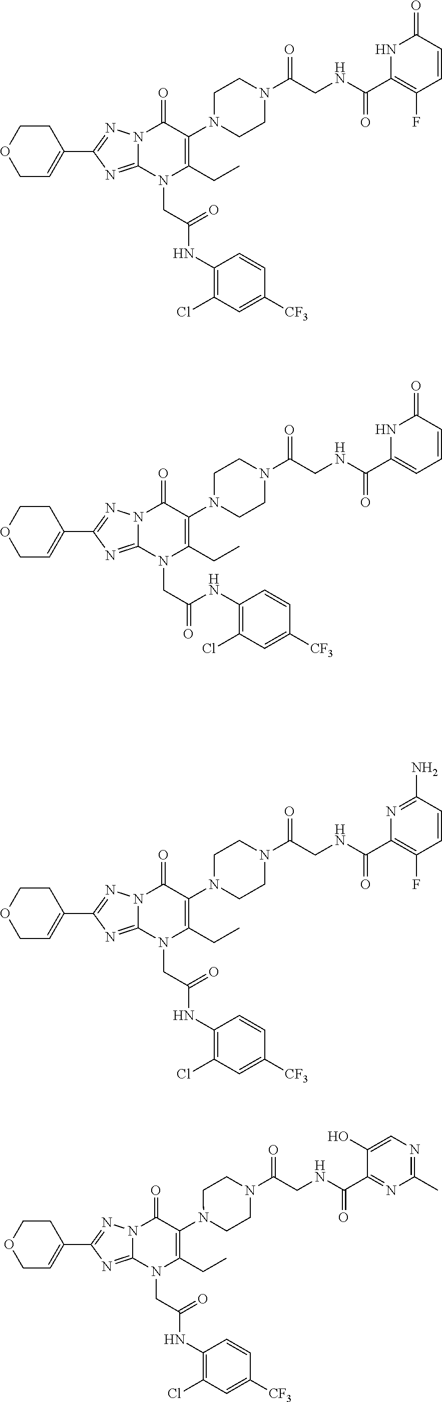 [SMILES: CCc1c(N2CCN(C(=O)CNC(=O)c3[nH]c(=O)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc(=O)[nH]3)CC2)c(=O)n2nc(C3=CCOCC3)nc2n1CC(=O)Nc1ccc(C(F)(F)F)cc1Cl ; CCc1c(N2CCN(C(=O)CNC(=O)c3nc(N)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3nc(C)ncc3O)CC2)c(=O)n2nc(C3=CCOCC3)nc2n1CC(=O)Nc1ccc(C(F)(F)F)cc1Cl] 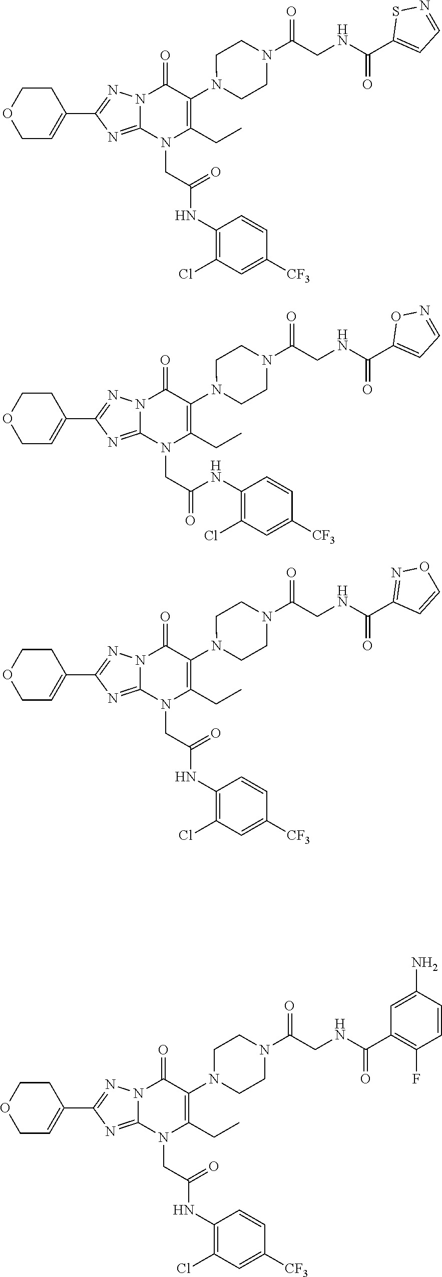 [SMILES: CCc1c(N2CCN(C(=O)CNC(=O)c3ccns3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccno3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccon3)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(N)ccc3F)CC2)c(=O)n2nc(C3=CCOCC3)nc2n1CC(=O)Nc1ccc(C(F)(F)F)cc1Cl] 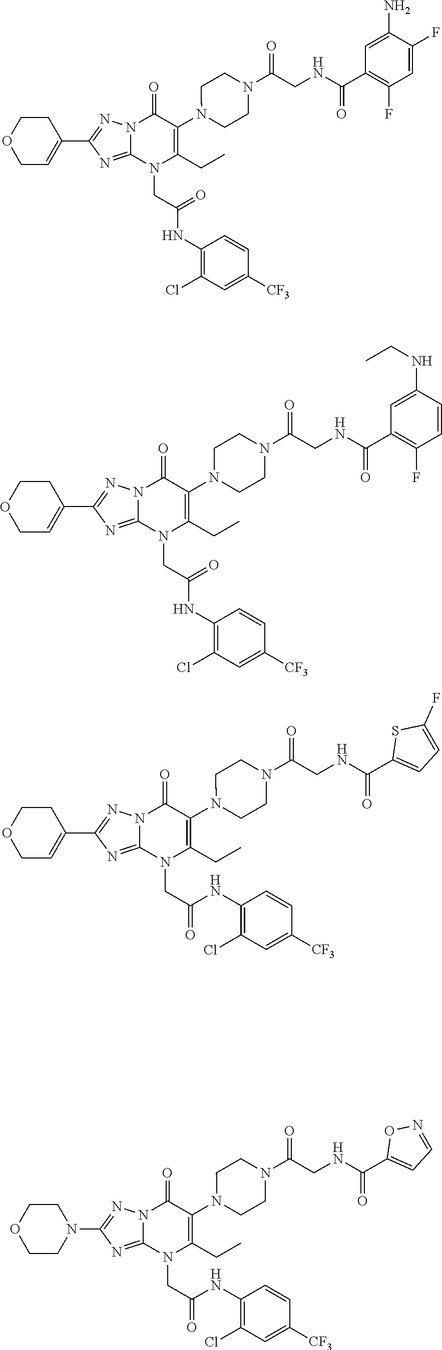 [SMILES: CCc1c(N2CCN(C(=O)CNC(=O)c3cc(N)c(F)cc3F)CC2)c(=O)n2nc(C3=CCOCC3)nc2n1CC(=O)Nc1ccc(C(F)(F)F)cc1Cl ; CCNc1ccc(F)c(C(=O)NCC(=O)N2CCN(c3c(CC)n(CC(=O)Nc4ccc(C(F)(F)F)cc4Cl)c4nc(C5=CCOCC5)nn4c3=O)CC2)c1 ; CCc1c(N2CCN(C(=O)CNC(=O)c3ccc(F)s3)CC2)c(=O)n2nc(C3=CCOCC3)nc2n1CC(=O)Nc1ccc(C(F)(F)F)cc1Cl ; CCc1c(N2CCN(C(=O)CNC(=O)c3ccno3)CC2)c(=O)n2nc(N3CCOCC3)nc2n1CC(=O)Nc1ccc(C(F)(F)F)cc1Cl] 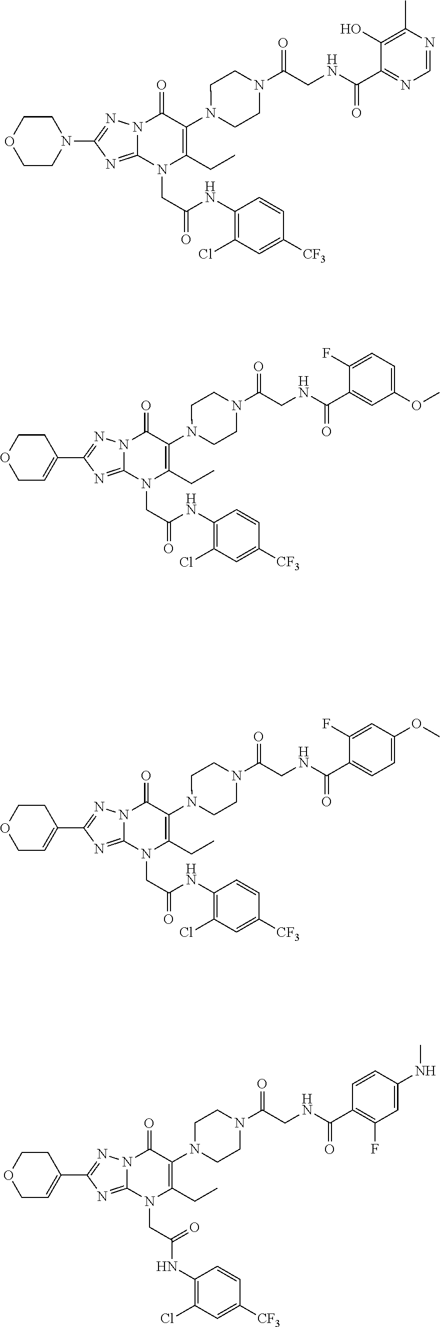 [SMILES: CCc1c(N2CCN(C(=O)CNC(=O)c3ncnc(C)c3O)CC2)c(=O)n2nc(N3CCOCC3)nc2n1CC(=O)Nc1ccc(C(F)(F)F)cc1Cl ; CCc1c(N2CCN(C(=O)CNC(=O)c3cc(OC)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(OC)cc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3ccc(NC)cc3F)CC2)c(=O)n2nc(C3=CCOCC3)nc2n1CC(=O)Nc1ccc(C(F)(F)F)cc1Cl] 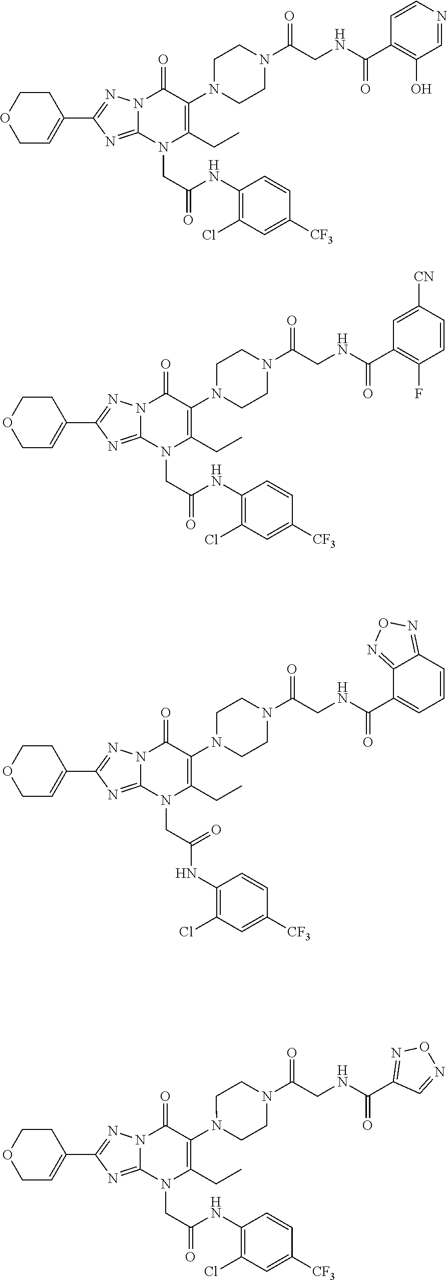 [SMILES: CCc1c(N2CCN(C(=O)CNC(=O)c3ccncc3O)CC2)c(=O)n2nc(C3=CCOCC3)nc2n1CC(=O)Nc1ccc(C(F)(F)F)cc1Cl ; CCc1c(N2CCN(C(=O)CNC(=O)c3cc(C#N)ccc3F)CC2)c(=O)n2nc(C3=CCOCC3)nc2n1CC(=O)Nc1ccc(C(F)(F)F)cc1Cl ; CCc1c(N2CCN(C(=O)CNC(=O)c3cccc4nonc34)CC2)c(=O)n2nc(C3=CCOCC3)nc2n1CC(=O)Nc1ccc(C(F)(F)F)cc1Cl ; CCc1c(N2CCN(C(=O)CNC(=O)c3cnon3)CC2)c(=O)n2nc(C3=CCOCC3)nc2n1CC(=O)Nc1ccc(C(F)(F)F)cc1Cl] 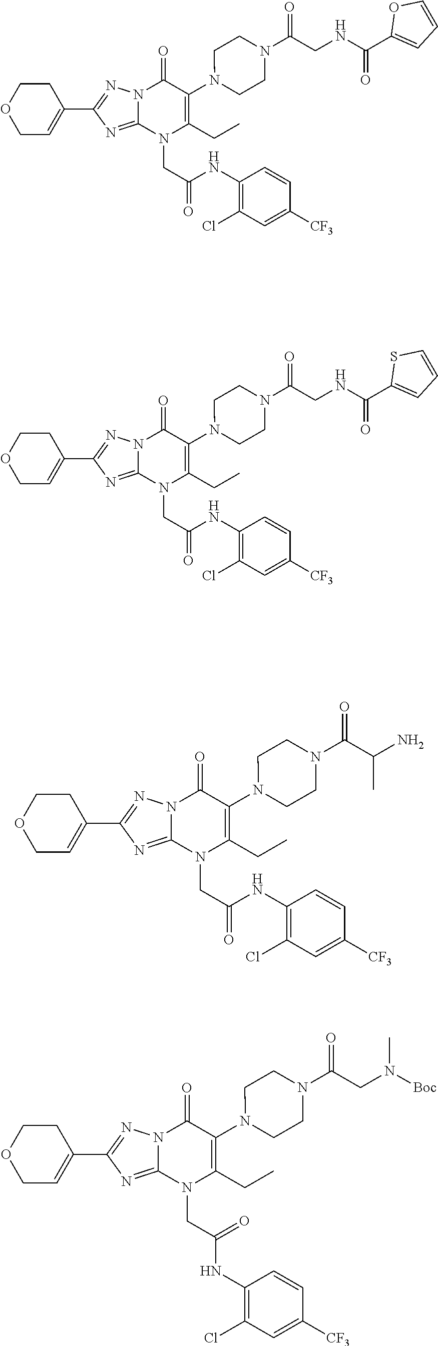 [SMILES: CCc1c(N2CCN(C(=O)CNC(=O)c3ccco3)CC2)c(=O)n2nc(C3=CCOCC3)nc2n1CC(=O)Nc1ccc(C(F)(F)F)cc1Cl ; CCc1c(N2CCN(C(=O)CNC(=O)c3cccs3)CC2)c(=O)n2nc(C3=CCOCC3)nc2n1CC(=O)Nc1ccc(C(F)(F)F)cc1Cl ; CCc1c(N2CCN(C(=O)C(C)N)CC2)c(=O)n2nc(C3=CCOCC3)nc2n1CC(=O)Nc1ccc(C(F)(F)F)cc1Cl ; CCc1c(N2CCN(C(=O)CN(C)C(=O)OC(C)(C)C)CC2)c(=O)n2nc(C3=CCOCC3)nc2n1CC(=O)Nc1ccc(C(F)(F)F)cc1Cl] 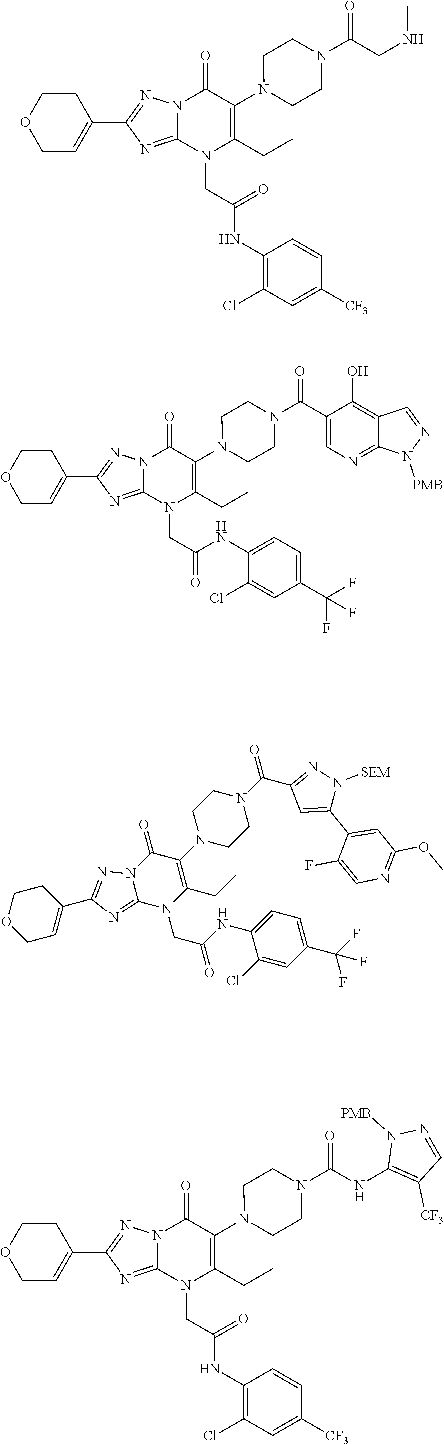 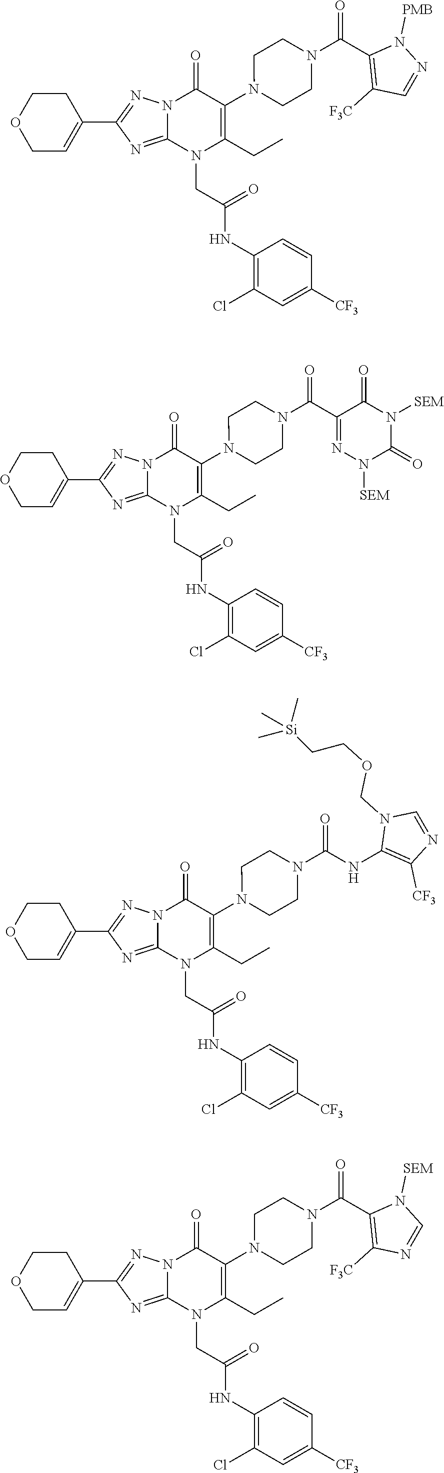 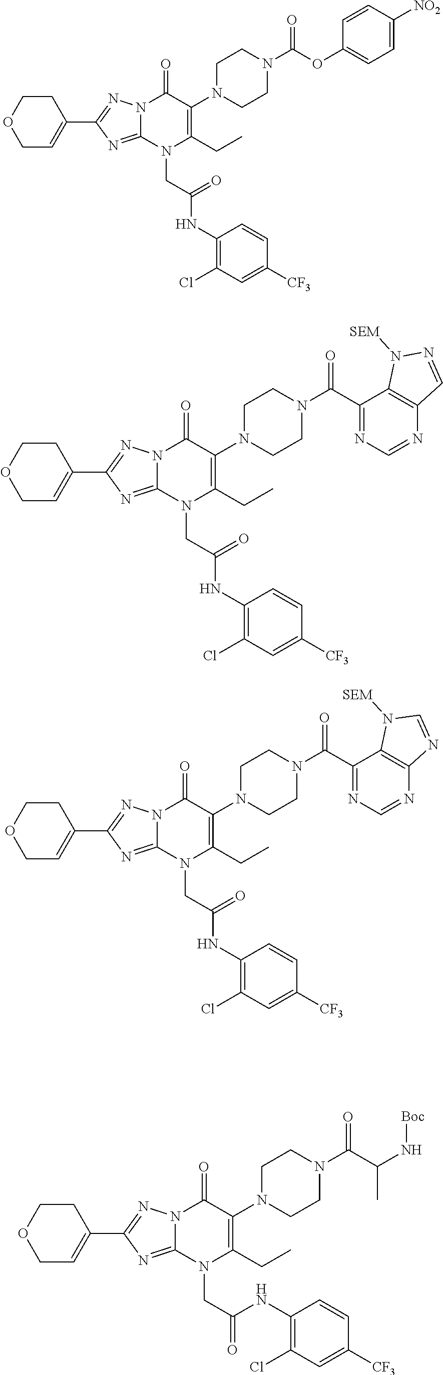 [SMILES: CCc1c(N2CCN(C(=O)Oc3ccc([N+](=O)[O-])cc3)CC2)c(=O)n2nc(C3=CCOCC3)nc2n1CC(=O)Nc1ccc(C(F)(F)F)cc1Cl ; CCc1c(N2CCN(C(=O)c3ncnc4cnn(COCC[Si](C)(C)C)c34)CC2)c(=O)n2nc(C3=CCOCC3)nc2n1CC(=O)Nc1ccc(C(F)(F)F)cc1Cl ; CCc1c(N2CCN(C(=O)c3ncnc4ncn(COCC[Si](C)(C)C)c34)CC2)c(=O)n2nc(C3=CCOCC3)nc2n1CC(=O)Nc1ccc(C(F)(F)F)cc1Cl ; CCc1c(N2CCN(C(=O)C(C)NC(=O)OC(C)(C)C)CC2)c(=O)n2nc(C3=CCOCC3)nc2n1CC(=O)Nc1ccc(C(F)(F)F)cc1Cl] 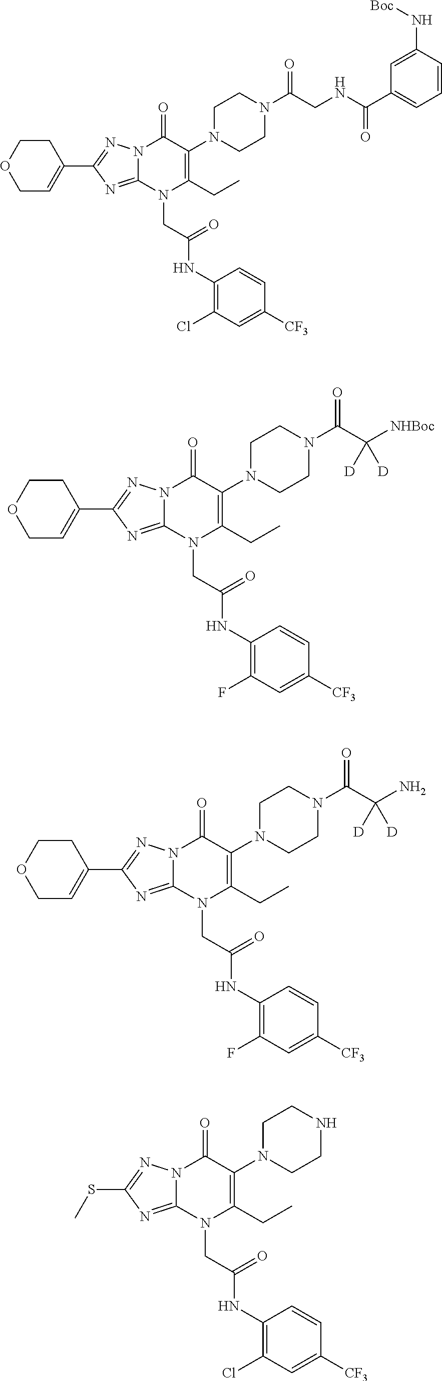 [SMILES: CCc1c(N2CCN(C(=O)CNC(=O)c3cccc(NC(=O)OC(C)(C)C)c3)CC2)c(=O)n2nc(C3=CCOCC3)nc2n1CC(=O)Nc1ccc(C(F)(F)F)cc1Cl ; [2H]C([2H])(NC(=O)OC(C)(C)C)C(=O)N1CCN(c2c(CC)n(CC(=O)Nc3ccc(C(F)(F)F)cc3F)c3nc(C4=CCOCC4)nn3c2=O)CC1 ; [2H]C([2H])(N)C(=O)N1CCN(c2c(CC)n(CC(=O)Nc3ccc(C(F)(F)F)cc3F)c3nc(C4=CCOCC4)nn3c2=O)CC1 ; CCc1c(N2CCNCC2)c(=O)n2nc(SC)nc2n1CC(=O)Nc1ccc(C(F)(F)F)cc1Cl] 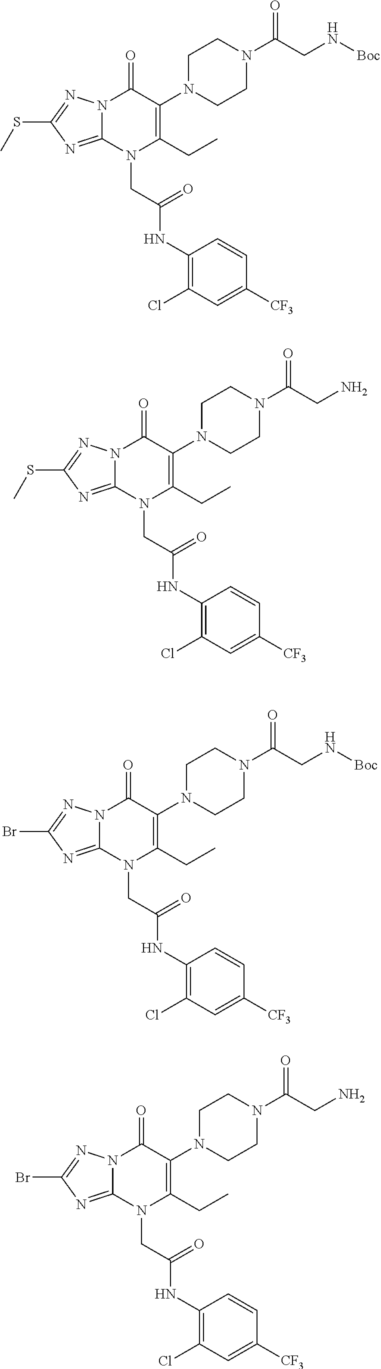 [SMILES: CCc1c(N2CCN(C(=O)CNC(=O)OC(C)(C)C)CC2)c(=O)n2nc(SC)nc2n1CC(=O)Nc1ccc(C(F)(F)F)cc1Cl ; CCc1c(N2CCN(C(=O)CN)CC2)c(=O)n2nc(SC)nc2n1CC(=O)Nc1ccc(C(F)(F)F)cc1Cl ; CCc1c(N2CCN(C(=O)CNC(=O)OC(C)(C)C)CC2)c(=O)n2nc(Br)nc2n1CC(=O)Nc1ccc(C(F)(F)F)cc1Cl ; CCc1c(N2CCN(C(=O)CN)CC2)c(=O)n2nc(Br)nc2n1CC(=O)Nc1ccc(C(F)(F)F)cc1Cl] 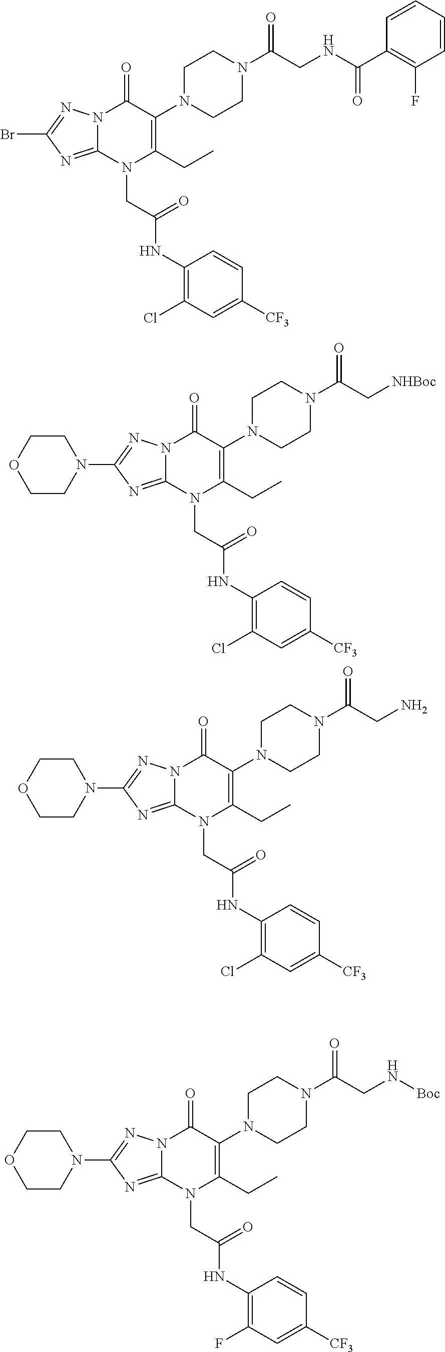 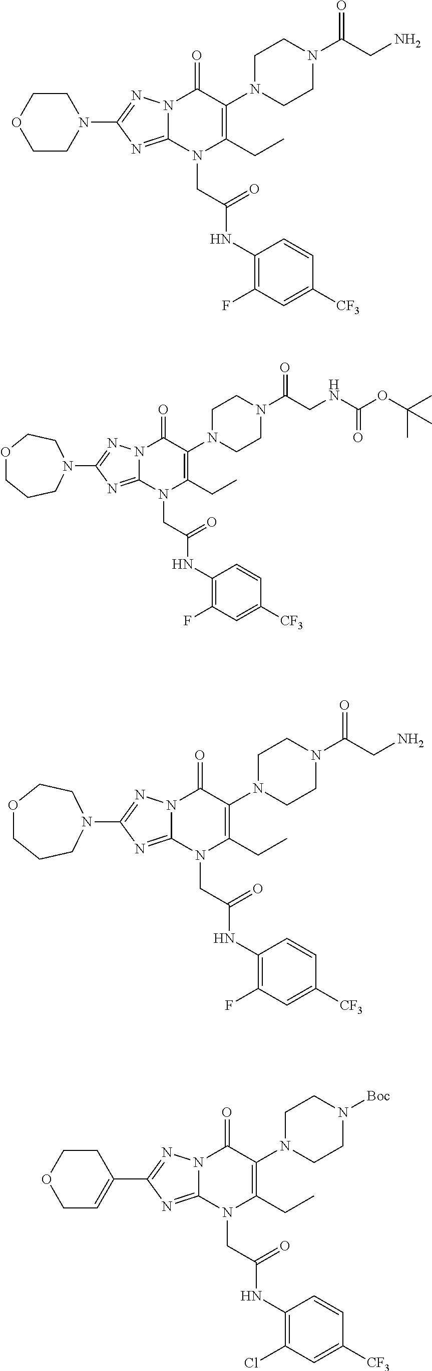 [SMILES: CCc1c(N2CCN(C(=O)CN)CC2)c(=O)n2nc(N3CCOCC3)nc2n1CC(=O)Nc1ccc(C(F)(F)F)cc1F ; CCc1c(N2CCN(C(=O)CNC(=O)OC(C)(C)C)CC2)c(=O)n2nc(N3CCCOCC3)nc2n1CC(=O)Nc1ccc(C(F)(F)F)cc1F ; CCc1c(N2CCN(C(=O)CN)CC2)c(=O)n2nc(N3CCCOCC3)nc2n1CC(=O)Nc1ccc(C(F)(F)F)cc1F ; CCc1c(N2CCN(C(=O)OC(C)(C)C)CC2)c(=O)n2nc(C3=CCOCC3)nc2n1CC(=O)Nc1ccc(C(F)(F)F)cc1Cl] 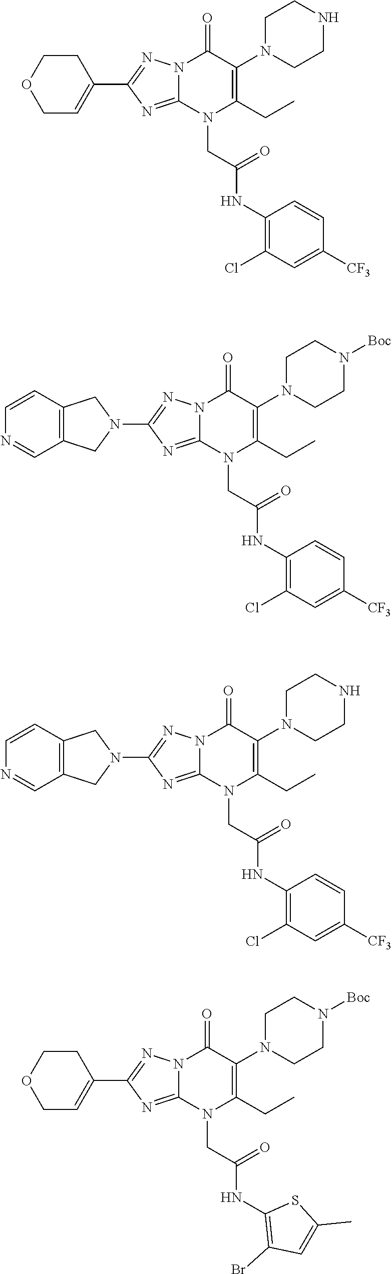 [SMILES: CCc1c(N2CCNCC2)c(=O)n2nc(C3=CCOCC3)nc2n1CC(=O)Nc1ccc(C(F)(F)F)cc1Cl ; CCc1c(N2CCN(C(=O)OC(C)(C)C)CC2)c(=O)n2nc(N3Cc4ccncc4C3)nc2n1CC(=O)Nc1ccc(C(F)(F)F)cc1Cl ; CCc1c(N2CCNCC2)c(=O)n2nc(N3Cc4ccncc4C3)nc2n1CC(=O)Nc1ccc(C(F)(F)F)cc1Cl ; CCc1c(N2CCN(C(=O)OC(C)(C)C)CC2)c(=O)n2nc(C3=CCOCC3)nc2n1CC(=O)Nc1sc(C)cc1Br] 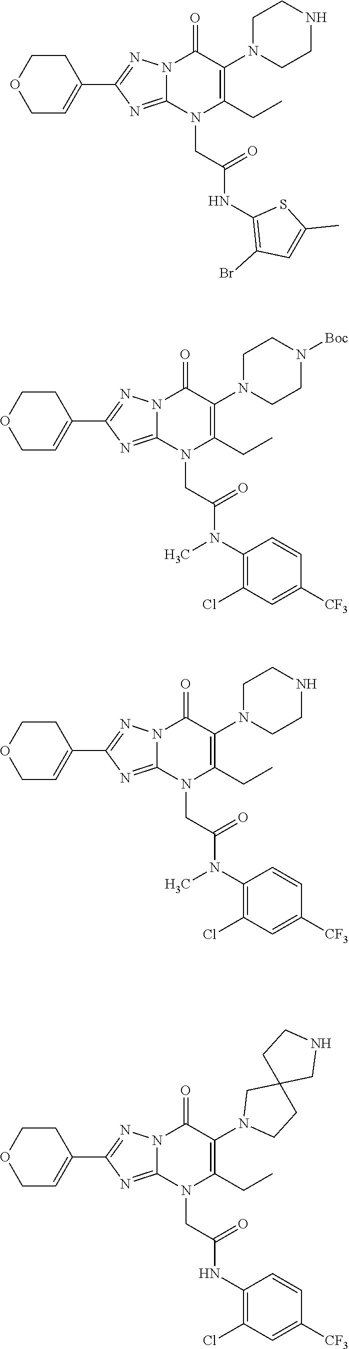 [SMILES: CCc1c(N2CCNCC2)c(=O)n2nc(C3=CCOCC3)nc2n1CC(=O)Nc1sc(C)cc1Br ; CCc1c(N2CCN(C(=O)OC(C)(C)C)CC2)c(=O)n2nc(C3=CCOCC3)nc2n1CC(=O)N(C)c1ccc(C(F)(F)F)cc1Cl ; CCc1c(N2CCNCC2)c(=O)n2nc(C3=CCOCC3)nc2n1CC(=O)N(C)c1ccc(C(F)(F)F)cc1Cl ; CCc1c(N2CCC3(CCNC3)C2)c(=O)n2nc(C3=CCOCC3)nc2n1CC(=O)Nc1ccc(C(F)(F)F)cc1Cl] 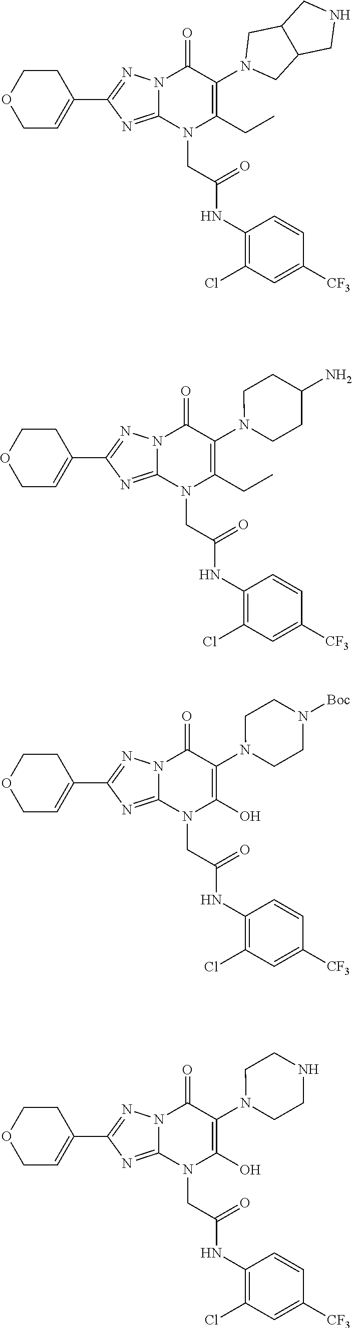 [SMILES: CCc1c(N2CC3CNCC3C2)c(=O)n2nc(C3=CCOCC3)nc2n1CC(=O)Nc1ccc(C(F)(F)F)cc1Cl ; CCc1c(N2CCC(N)CC2)c(=O)n2nc(C3=CCOCC3)nc2n1CC(=O)Nc1ccc(C(F)(F)F)cc1Cl ; CC(C)(C)OC(=O)N1CCN(c2c(O)n(CC(=O)Nc3ccc(C(F)(F)F)cc3Cl)c3nc(C4=CCOCC4)nn3c2=O)CC1 ; O=C(Cn1c(O)c(N2CCNCC2)c(=O)n2nc(C3=CCOCC3)nc12)Nc1ccc(C(F)(F)F)cc1Cl] 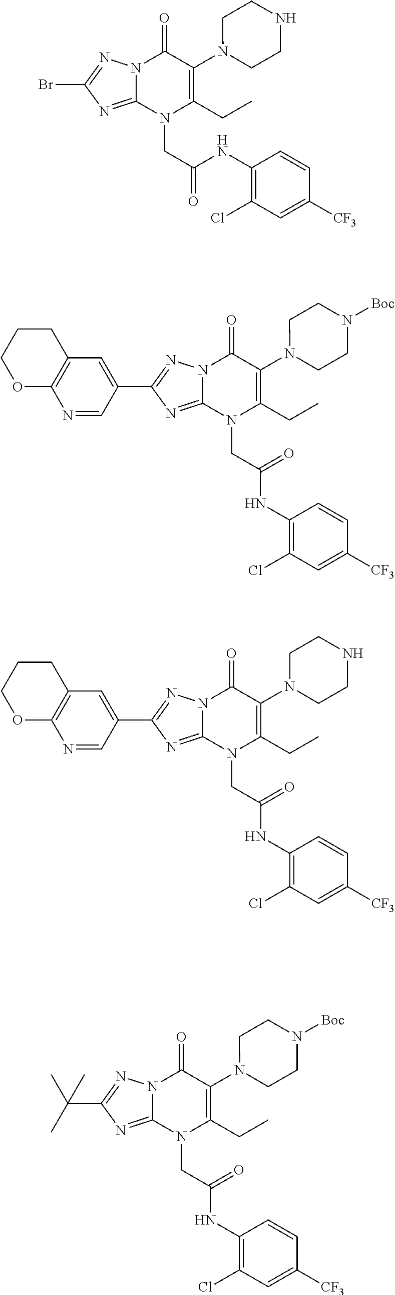 [SMILES: CCc1c(N2CCNCC2)c(=O)n2nc(Br)nc2n1CC(=O)Nc1ccc(C(F)(F)F)cc1Cl ; CCc1c(N2CCN(C(=O)OC(C)(C)C)CC2)c(=O)n2nc(-c3cnc4c(c3)CCCO4)nc2n1CC(=O)Nc1ccc(C(F)(F)F)cc1Cl ; CCc1c(N2CCNCC2)c(=O)n2nc(-c3cnc4c(c3)CCCO4)nc2n1CC(=O)Nc1ccc(C(F)(F)F)cc1Cl ; CCc1c(N2CCN(C(=O)OC(C)(C)C)CC2)c(=O)n2nc(C(C)(C)C)nc2n1CC(=O)Nc1ccc(C(F)(F)F)cc1Cl] 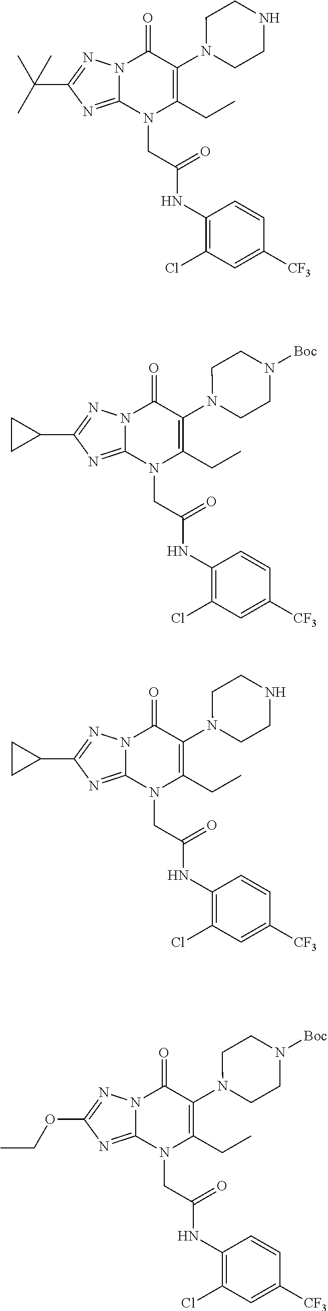 [SMILES: CCc1c(N2CCNCC2)c(=O)n2nc(C(C)(C)C)nc2n1CC(=O)Nc1ccc(C(F)(F)F)cc1Cl ; CCc1c(N2CCN(C(=O)OC(C)(C)C)CC2)c(=O)n2nc(C3CC3)nc2n1CC(=O)Nc1ccc(C(F)(F)F)cc1Cl ; CCc1c(N2CCNCC2)c(=O)n2nc(C3CC3)nc2n1CC(=O)Nc1ccc(C(F)(F)F)cc1Cl ; CCOc1nc2n(CC(=O)Nc3ccc(C(F)(F)F)cc3Cl)c(CC)c(N3CCN(C(=O)OC(C)(C)C)CC3)c(=O)n2n1] 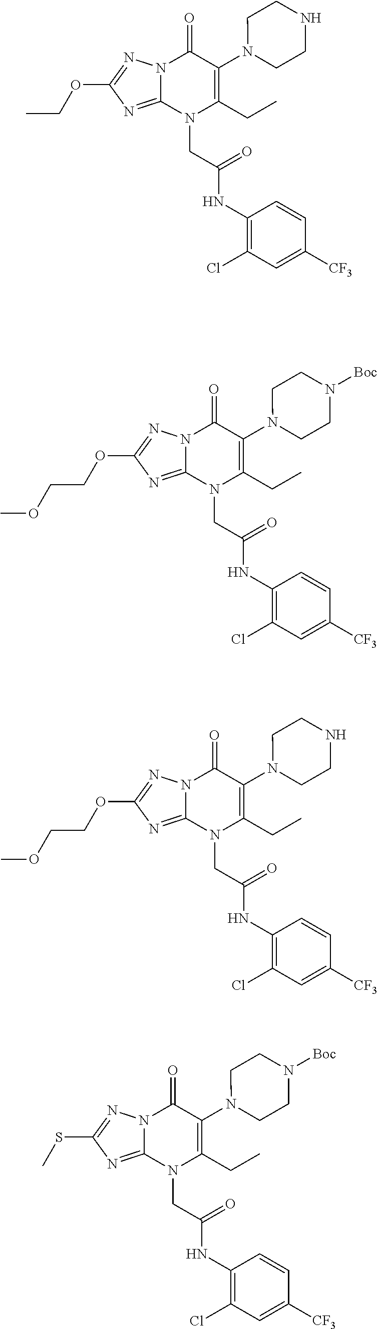 [SMILES: CCOc1nc2n(CC(=O)Nc3ccc(C(F)(F)F)cc3Cl)c(CC)c(N3CCNCC3)c(=O)n2n1 ; CCc1c(N2CCN(C(=O)OC(C)(C)C)CC2)c(=O)n2nc(OCCOC)nc2n1CC(=O)Nc1ccc(C(F)(F)F)cc1Cl ; CCc1c(N2CCNCC2)c(=O)n2nc(OCCOC)nc2n1CC(=O)Nc1ccc(C(F)(F)F)cc1Cl ; CCc1c(N2CCN(C(=O)OC(C)(C)C)CC2)c(=O)n2nc(SC)nc2n1CC(=O)Nc1ccc(C(F)(F)F)cc1Cl] 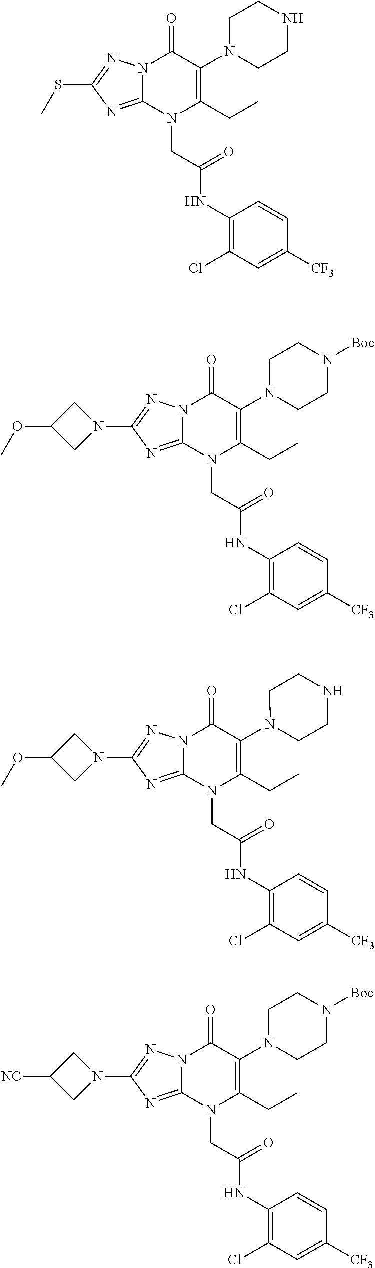 [SMILES: CCc1c(N2CCNCC2)c(=O)n2nc(SC)nc2n1CC(=O)Nc1ccc(C(F)(F)F)cc1Cl ; CCc1c(N2CCN(C(=O)OC(C)(C)C)CC2)c(=O)n2nc(N3CC(OC)C3)nc2n1CC(=O)Nc1ccc(C(F)(F)F)cc1Cl ; CCc1c(N2CCNCC2)c(=O)n2nc(N3CC(OC)C3)nc2n1CC(=O)Nc1ccc(C(F)(F)F)cc1Cl ; CCc1c(N2CCN(C(=O)OC(C)(C)C)CC2)c(=O)n2nc(N3CC(C#N)C3)nc2n1CC(=O)Nc1ccc(C(F)(F)F)cc1Cl] 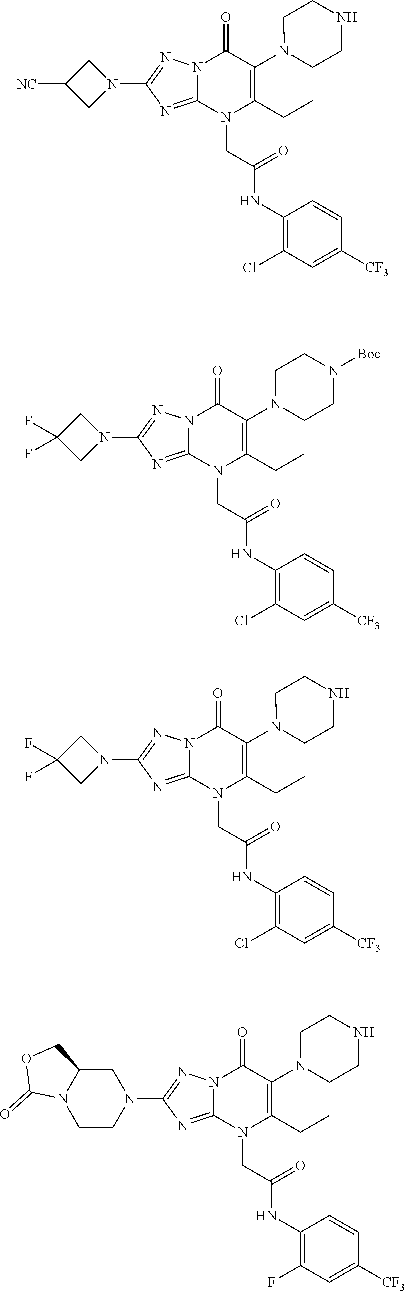 [SMILES: CCc1c(N2CCNCC2)c(=O)n2nc(N3CC(C#N)C3)nc2n1CC(=O)Nc1ccc(C(F)(F)F)cc1Cl ; CCc1c(N2CCN(C(=O)OC(C)(C)C)CC2)c(=O)n2nc(N3CC(F)(F)C3)nc2n1CC(=O)Nc1ccc(C(F)(F)F)cc1Cl ; CCc1c(N2CCNCC2)c(=O)n2nc(N3CC(F)(F)C3)nc2n1CC(=O)Nc1ccc(C(F)(F)F)cc1Cl ; CCc1c(N2CCNCC2)c(=O)n2nc(N3CCN4C(=O)OC[C@H]4C3)nc2n1CC(=O)Nc1ccc(C(F)(F)F)cc1F] 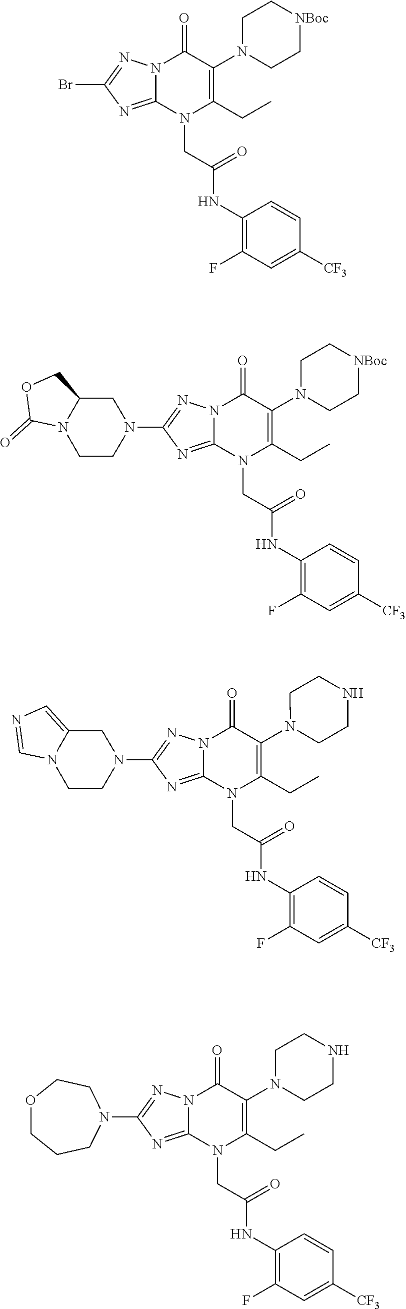 [SMILES: CCc1c(N2CCN(C(=O)OC(C)(C)C)CC2)c(=O)n2nc(Br)nc2n1CC(=O)Nc1ccc(C(F)(F)F)cc1F ; CCc1c(N2CCN(C(=O)OC(C)(C)C)CC2)c(=O)n2nc(N3CCN4C(=O)OC[C@H]4C3)nc2n1CC(=O)Nc1ccc(C(F)(F)F)cc1F ; CCc1c(N2CCNCC2)c(=O)n2nc(N3CCn4cncc4C3)nc2n1CC(=O)Nc1ccc(C(F)(F)F)cc1F ; CCc1c(N2CCNCC2)c(=O)n2nc(N3CCCOCC3)nc2n1CC(=O)Nc1ccc(C(F)(F)F)cc1F] 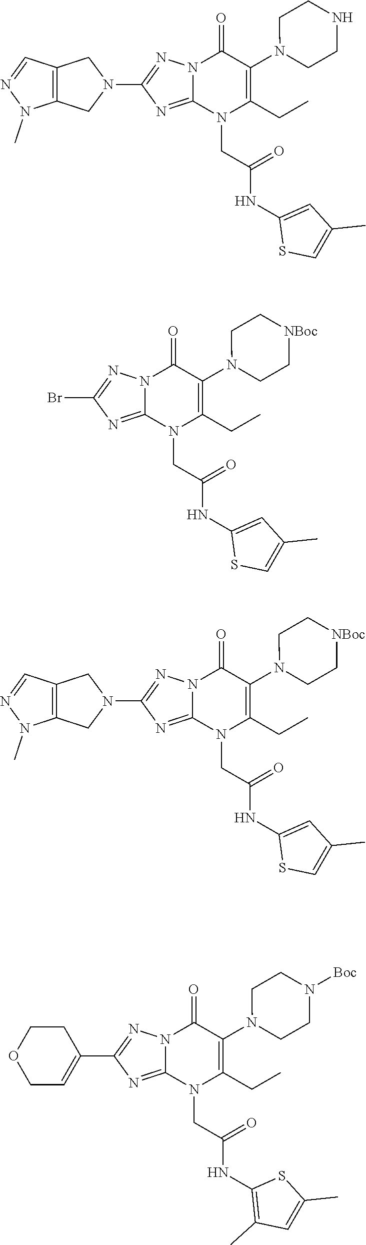 [SMILES: CCc1c(N2CCNCC2)c(=O)n2nc(N3Cc4cnn(C)c4C3)nc2n1CC(=O)Nc1cc(C)cs1 ; CCc1c(N2CCN(C(=O)OC(C)(C)C)CC2)c(=O)n2nc(Br)nc2n1CC(=O)Nc1cc(C)cs1 ; CCc1c(N2CCN(C(=O)OC(C)(C)C)CC2)c(=O)n2nc(N3Cc4cnn(C)c4C3)nc2n1CC(=O)Nc1cc(C)cs1 ; CCc1c(N2CCN(C(=O)OC(C)(C)C)CC2)c(=O)n2nc(C3=CCOCC3)nc2n1CC(=O)Nc1sc(C)cc1C] 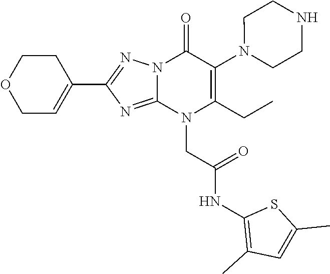 [SMILES: CCc1c(N2CCNCC2)c(=O)n2nc(C3=CCOCC3)nc2n1CC(=O)Nc1sc(C)cc1C]
or a pharmaceutically acceptable salt thereof.

28. A pharmaceutical composition comprising the compound of claim 1 or the pharmaceutically acceptable salt thereof, and a pharmaceutically acceptable carrier or excipient.

29. A method of treating a disease which can be treated by WRN inhibition in a subject, comprising administering to the subject a therapeutically effective amount of the compound of claim 1, or the pharmaceutically acceptable salt thereof; wherein the disease is cancer; and/or, the cancer is characterized as MSI-H or dMMR; and/or, the cancer is colorectal, gastric, prostate, endometrial, adrenocortical, uterine, cervical, esophageal, breast, kidney and ovarian cancer.

30. A method of treating a disease which can be treated by WRN inhibition in a subject, comprising administering to the subject a therapeutically effective amount of the pharmaceutical composition of claim 28; wherein the disease is cancer; and/or, the cancer is characterized as MSI-H or dMMR; and/or, the cancer is colorectal, gastric, prostate, endometrial, adrenocortical, uterine, cervical, esophageal, breast, kidney and ovarian cancer.
